SEQUENCE LISTING (70 sequences)

SEQ ID NO: 1 - protein, 5 aa, synthetic construct
SYGMH

SEQ ID NO: 2 - protein, 16 aa, synthetic construct
VIWEDGMKYYTDSVKG

SEQ ID NO: 3 - protein, 15 aa, synthetic construct
DRGIGARRGPYYMDV

SEQ ID NO: 4 - protein, 11 aa, synthetic construct
RASQSISSYLN

SEQ ID NO: 5 - protein, 7 aa, synthetic construct
AASSLQS

SEQ ID NO: 6 - protein, 9 aa, synthetic construct
QQSYSTPLI

SEQ ID NO: 7 - protein, 124 aa, synthetic construct
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVYVSS

SEQ ID NO: 8 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKR

SEQ ID NO: 9 - protein, 453 aa, synthetic construct
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 8 aa, synthetic construct
GFTFSRYS

SEQ ID NO: 12 - protein, 8 aa, synthetic construct
INSVGNST

SEQ ID NO: 13 - protein, 5 aa, synthetic construct
ASGDY

SEQ ID NO: 14 - protein, 11 aa, synthetic construct
QSLIYSDGNAY

SEQ ID NO: 15 - skipped

SEQ ID NO: 16 - protein, 9 aa, synthetic construct
SQSTHVPWT

SEQ ID NO: 17 - protein, 97 aa, synthetic construct
EVQLVESGGGLVQPGGSLRLSCAASGFTFSRYSMSWVRQAPGKGLELVAQINSVGNSTYYPDTVKGRFTISRDNAKNTLYLQMNSLRAEDTAVYYCA

SEQ ID NO: 18 - protein, 100 aa, synthetic construct
DVVMTQSPLSLPVTLGQPASISCRSSQSLIYSDGNAYLHWFLQKPGQSPRLLIYKVSNRFSGVPDRFSGSGSGTDFTLKISRVEAEDVGVYYCSQSTHVP

SEQ ID NO: 19 - protein, 442 aa, synthetic construct
EVQLVESGGGLVQPGGSLRLSCAASGFTFSRYSMSWVRQAPGKGLELVAQINSVGNSTYYPDTVKGRFTISRDNAKNTLYLQMNSLRAEDTAVYYCASGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 219 aa, synthetic construct
DVVMTQSPLSLPVTLGQPASISCRSSQSLIYSDGNAYLHWFLQKPGQSPRLLIYKVSNRFSGVPDRFSGSGSGTDFTLKISRVEAEDVGVYYCSQSTHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 8 aa, synthetic construct
GFTFSSYG

SEQ ID NO: 22 - protein, 8 aa, synthetic construct
INSNGGST

SEQ ID NO: 23 - protein, 5 aa, synthetic construct
ASGDY

SEQ ID NO: 24 - protein, 11 aa, synthetic construct
QSLVYSNGDTY

SEQ ID NO: 25 - skipped

SEQ ID NO: 26 - protein, 9 aa, synthetic construct
SQSTHVPWT

SEQ ID NO: 27 - protein, 97 aa, synthetic construct
EVQLVESGGGLVQPGGSLRLSCAASGFTFSSYGMSWVRQAPGKGLELVASINSNGGSTYYPDSVKGRFTISRDNAKNSLYLQMNSLRAEDTAVYYCA

SEQ ID NO: 28 - protein, 99 aa, synthetic construct
DIVMTQSPLSLPVTPGEPASISCRSSQSLVYSNGDTYLHWYLQKPGQSPQLLIYKVSNRFSGVPDRFSGSGSGTDFTLKISRVEAEDVGVYYCSQSTHV

SEQ ID NO: 29 - protein, 440 aa, synthetic construct
EVQLVESGGGLVQPGGSLRLSCAASGFTFSSYGMSWVRQAPGKGLELVASINSNGGSTYYPDSVKGRFTISRDNAKNSLYLQMNSLRAEDTAVYYCASGDYWGQGTTVTVSSASTKGPSVFPLAPCSRSTSESTAALGCLVKDYFPEPVTVSWNSGALTSGVHTFPAVLQSSGLYSLSSVCHVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 30 - protein, 219 aa, synthetic construct
DIVMTQSPLSLPVTPGEPASISCRSSQSLVYSNGDTYLHWYLQKPGQSPQLLIYKVSNRFSGVPDRFSGSGSGTDFTLKISRVEAEDVGVYYCSQSTHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 8 aa, synthetic construct
GFTFSSYA

SEQ ID NO: 32 - protein, 8 aa, synthetic construct
INASGTRT

SEQ ID NO: 33 - protein, 19 aa, synthetic construct
ARGKGNTHKPYGYVRYFDV

SEQ ID NO: 34 - protein, 7 aa, synthetic construct
QSVSSSY

SEQ ID NO: 35 - skipped

SEQ ID NO: 36 - protein, 9 aa, synthetic construct
LQIYNMPIT

SEQ ID NO: 37 - protein, 93 aa, synthetic construct
QVELVESGGGLVQPGGSLRLSCAASGFTFSSYAMSWVRQAPGKGLEWVSAINASGTRTYYADSVKGRFTISRDNSKNTLYLQMNSLRAEDTAV

SEQ ID NO: 38 - protein, 95 aa, synthetic construct
IVLTQSPATLSLSPGERATLSCRASQSVSSSYLAWYQQKPGQAPRLLIYGASSRATGVPARFSGSGSGTDFTLTISSLEPEDFATYYCLQIYNMP

SEQ ID NO: 39 - protein, 456 aa, synthetic construct
QVELVESGGGLVQPGGSLRLSCAASGFTFSSYAMSWVRQAPGKGLEWVSAINASGTRTYYADSVKGRFTISRDNSKNTLYLQMNSLRAEDTAVYYCARGKGNTHKPYGYVRYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 215 aa, synthetic construct
DIVLTQSPATLSLSPGERATLSCRASQSVSSSYLAWYQQKPGQAPRLLIYGASSRATGVPARFSGSGSGTDFTLTISSLEPEDFATYYCLQIYNMPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 8 aa, synthetic construct
GYYTEAYY

SEQ ID NO: 42 - protein, 8 aa, synthetic construct
IDPATGNT

SEQ ID NO: 43 - protein, 9 aa, synthetic construct
ASLYSLPVY

SEQ ID NO: 44 - protein, 11 aa, synthetic construct
QSLLYSDAKTY

SEQ ID NO: 45 - skipped

SEQ ID NO: 46 - protein, 9 aa, synthetic construct
LQGTHYPVL

SEQ ID NO: 47 - protein, 97 aa, synthetic construct
QVQLVQSGAEVKKPGASVKVSCKASGYYTEAYYIHWVRQAPGQGLEWMGRIDPATGNTKYAPRLQDRVTMTRDTSTSTVYMELSSLRSEDTAVYYCA

SEQ ID NO: 48 - protein, 100 aa, synthetic construct
DVVMTQSPLSLPVTLGQPASISCKSSQSLLYSDAKTYLNWFQQRPGQSPRRLIYQISRLDPGVPDRFSGSGSGTDFTLKISRVEAEDVGVYYCLQGTHYP

SEQ ID NO: 49 - protein, 442 aa, synthetic construct
QVQLVQSGAEVKKPGASVKVSCKASGYYTEAYYIHWVRQAPGQGLEWMGRIDPATGNTKYAPRLQDRVTMTRDTSTSTVYMELSSLRSEDTAVYYCASLYSLPVYWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 50 - protein, 219 aa, synthetic construct
DVVMTQSPLSLPVTLGQPASISCKSSQSLLYSDAKTYLNWFQQRPGQSPRRLIYQISRLDPGVPDRFSGSGSGTDFTLKISRVEAEDVGVYYCLQGTHYPVLFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 8 aa, synthetic construct
GFTFSNYG

SEQ ID NO: 52 - protein, 10 aa, synthetic construct
ASIRSGGGRT

SEQ ID NO: 53 - protein, 12 aa, synthetic construct
VRYDHYSGSSDY

SEQ ID NO: 54 - protein, 11 aa, synthetic construct
QSLLDSDGKTY

SEQ ID NO: 55 - skipped

SEQ ID NO: 56 - protein, 9 aa, synthetic construct
WQGTHFPRT

SEQ ID NO: 57 - protein, 98 aa, synthetic construct
EVQLLESGGGLVQPGGSLRLSCAASGFTFSNYGMSWVRQAPGKGLEWVASIRSGGGRTYYSDNVKGRFTISRDNSKNTLYLQMNSLRAEDTAVYYCVR

SEQ ID NO: 58 - protein, 100 aa, synthetic construct
DVVMTQSPLSLPVTPGEPASISCKSSQSLLDSDGKTYLNWLLQKPGQSPQRLIYLVSKLDSGVPDRFSGSGSGTDFTLKISRVEAEDVGVYYCWQGTHFP

SEQ ID NO: 59 - protein, 447 aa, synthetic construct
EVQLLESGGGLVQPGGSLRLSCAASGFTFSNYGMSWVRQAPGKGLEWVASIRSGGGRTYYSDNVKGRFTISRDNSKNTLYLQMNSLRAEDTAVYYCVRYDHYSGSSDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNKYTQKSLSLSPG

SEQ ID NO: 60 - protein, 180 aa, synthetic construct
DVVMTQSPLSLPVTPGEPASISCKSSQSLLDSDGKTYLNWLLQKPGQSPQRLIYLVSKLDSGVPDRFSGSGSGTDFTLKISRVEAEDVGVYYCWQGTHFPRTFGQGTKVEIKRTVAAPSVFIFPPSDEQLKSGTASVVCLLNNFYPREAKVQWKVDNALQSGNSQESVTEQDSKDSTYSL

SEQ ID NO: 61 - protein, 8 aa, synthetic construct
GFTFSSFG

SEQ ID NO: 62 - protein, 8 aa, synthetic construct
ISSGSSTI

SEQ ID NO: 63 - protein, 17 aa, synthetic construct
AREGGYYYGRSYYTMDY

SEQ ID NO: 64 - protein, 11 aa, synthetic construct
QSIVHSNGNTY

SEQ ID NO: 65 - skipped

SEQ ID NO: 66 - protein, 9 aa, synthetic construct
FQGSHVPPT

SEQ ID NO: 67 - protein, 98 aa, synthetic construct
EVQLVESGGGLVQPGGSLRLSCSASGFTFSSFGMHWVRQAPGKGLEWVAYISSGSSTIYYGDTVKGRFTISRDNAKNSLFLQMSSLRAEDTAVYYCAR

SEQ ID NO: 68 - protein, 100 aa, synthetic construct
DVVMTQSPLSLPVTPGAPASISCRSSQSIVHSNGNTYLEWYLQKPGQSPKLLIYKVSNRFSGVPDRFSGSGSGTDFTLRISRVEAEDVGIYYCFQGSHVP

SEQ ID NO: 69 - protein, 454 aa, synthetic construct
EVQLVESGGGLVQPGGSLRLSCSASGFTFSSFGMHWVRQAPGKGLEWVAYISSGSSTIYYGDTVKGRFTISRDNAKNSLFLQMSSLRAEDTAVYYCAREGGYYYGRSYYTMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 219 aa, synthetic construct
DVVMTQSPLSLPVTPGAPASISCRSSQSIVHSNGNTYLEWYLQKPGQSPKLLIYKVSNRFSGVPDRFSGSGSGTDFTLRISRVEAEDVGIYYCFQGSHVPPTFGPGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0368214 A1
(43) Pub. Date: Nov. 7, 2024

(54) POSITIVE NMDA-MODULATING COMPOUNDS AND METHODS OF USE THEREOF

(71) Applicant: SAGE THERAPEUTICS, INC., Cambridge, MA (US)

(72) Inventor: Maria-Jesus Blanco-Pillado, Arlington, MA (US)

(21) Appl. No.: 18/685,603

(22) PCT Filed: Aug. 25, 2022
(86) PCT No.: PCT/US2022/041603

§ 371 Date: Feb. 22, 2024

Related U.S. Application Data
(60) Provisional application No. 63/240,699, filed on Sep. 3, 2021.
(60) Provisional application No. 63/240,700, filed on Sep. 3, 2021.
(60) Provisional application No. 63/240,701, filed on Sep. 3, 2021.
(60) Provisional application No. 63/237,126, filed on Aug. 25, 2021.
(60) Provisional application No. 63/237,127, filed on Aug. 25, 2021.
(60) Provisional application No. 63/237,128, filed on Aug. 25, 2021.
(60) Provisional application No. 63/237,129, filed on Aug. 25, 2021.
(60) Provisional application No. 63/237,130, filed on Aug. 25, 2021.

(51) Int. Cl. C07J 63/00; A61K 31/575; A61K 31/58; C07J 9/00; C07J 17/00; C07J 41/00; C07J 53/00

(52) U.S. Cl. CPC C07J 63/002; A61K 31/575; A61K 31/58; C07J 9/00; C07J 17/00; C07J 41/0094; C07J 53/008; C07J 63/008

(57) ABSTRACT

Compounds are provided according to Formulae (A-I), (B-I), (C-I), (D-I), (E-I), (F-I), (G-I), and (H-I): and pharmaceutically acceptable salts thereof, and pharmaceutical compositions thereof. Compounds of the present disclosure are contemplated useful for the prevention and treatment of a variety of CNS-related conditions.

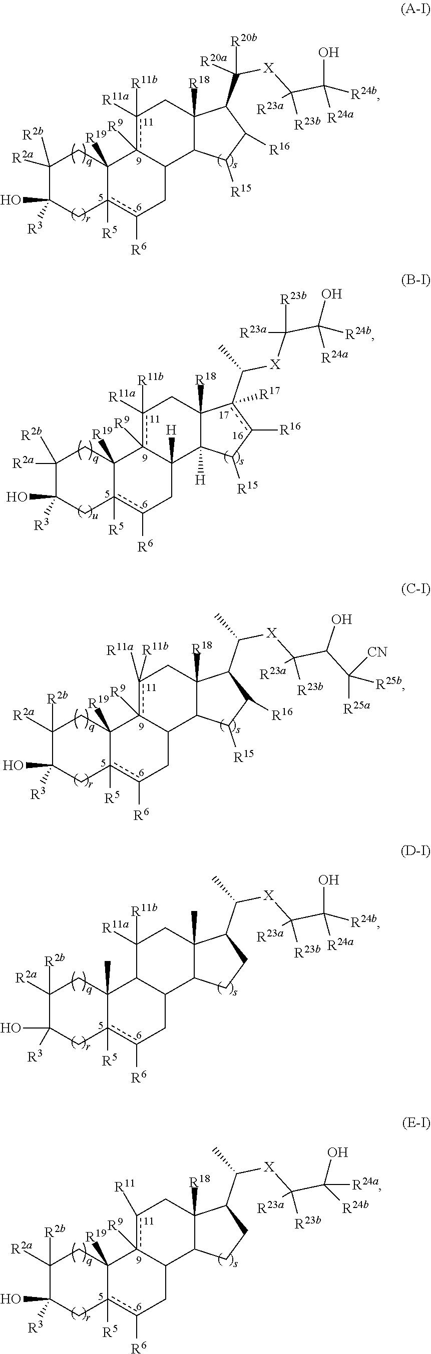

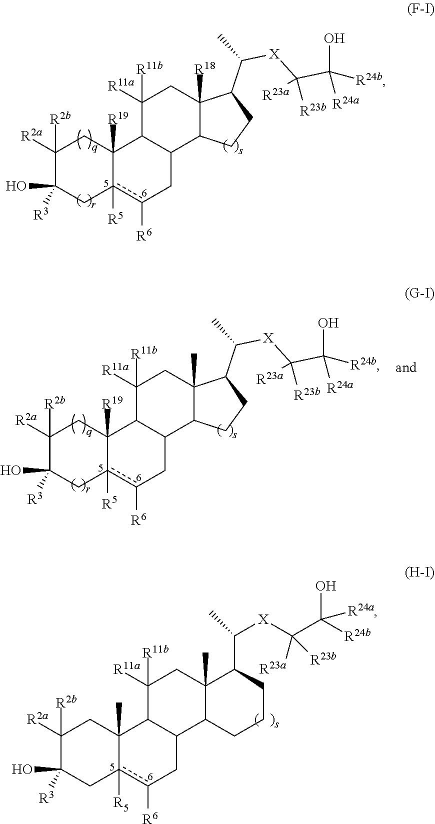

### RELATED APPLICATIONS

This application claims the benefit of and priority from U.S. Provisional Application Nos. 63/237,126, filed Aug. 25, 2021, 63/237,127, filed Aug. 25, 2021, 63/237,128, filed Aug. 25, 2021, 63/237,129, filed Aug. 25, 2021, 63/237,130, filed Aug. 25, 2021, 63/240,699, filed Sep. 3, 2021, 63/240,700, filed Sep. 3, 2021 and 63/240,701, filed Sep. 3, 2021. The contents of each of the foregoing applications are incorporated by reference herein in their entirety.

### BACKGROUND

NMDA receptors are heteromeric complexes comprised of NR1, NR2, and/or NR3 subunits and possess distinct recognition sites for exogenous and endogenous ligands. These recognition sites include binding sites for glycine, and glutamate agonists and modulators. NMDA receptors are expressed in the peripheral tissues and the CNS, where they are involved in excitatory synaptic transmission. Activating these receptors contributes to synaptic plasticity in some circumstances and excitotoxicity in others. These receptors are ligand-gated ion channels that admit Ca²⁺ after binding of the glutamate and glycine, and are fundamental to excitatory neurotransmission and normal CNS function. Positive modulators may be useful as therapeutic agents with potential clinical uses as cognitive enhancers and in the treatment of psychiatric disorders in which glutamatergic transmission is reduced or defective (see, e.g., Horak et al., J. of Neuroscience, 2004, 24 (46), 10318-10325).

There is a need for new compounds that are positive allosteric modulators of the NMDA receptor for the prevention and treatment of conditions associated with NMDA receptor function. Compounds, compositions, and methods described herein are directed toward this end.

### SUMMARY

The present disclosure provides compounds that are NMDA receptor positive allosteric modulators. In one aspect, the disclosure provides a compound of Formula (A-I):

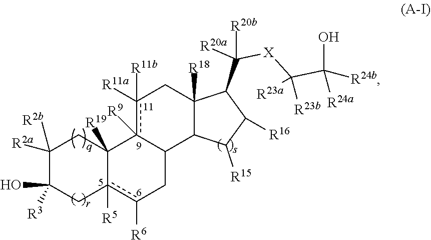

or a pharmaceutically acceptable salt thereof;
wherein:
   each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein R^{A2} is hydrogen or substituted or unsubstituted C₁₋₆alkyl;
   R³ is substituted or unsubstituted C₁₋₆alkyl, substituted or unsubstituted C₂₋₆ alkenyl, substituted or unsubstituted C₂₋₆alkynyl, or substituted or unsubstituted C₃₋₁₀carbocyclyl;
   R⁵ is absent, hydrogen, halogen, or substituted or unsubstituted C₁₋₆alkyl;
   R⁶ is hydrogen, halogen, or substituted or unsubstituted C₁₋₆alkyl;
   R⁹ is absent or hydrogen; or
   R⁹ and R^{11a} are taken together with the carbon atoms to which they are attached to form a substituted or unsubstituted C₃₋₆carbocyclyl ring;
   R^{11a} is hydrogen, halogen, substituted or unsubstituted C₁₋₆alkyl, or —OR^{A11}, wherein R^{A11} is hydrogen or substituted or unsubstituted C₁₋₆alkyl;
   R^{11b} is absent or hydrogen; or
   R^{11a} and R^{11b} are joined to form an oxo (═O) group;
   each instance of R¹⁵ is hydrogen, halogen, —OH, —NH₂, substituted or unsubstituted C₁₋₆alkyl, substituted or unsubstituted C₂₋₆alkenyl, substituted or unsubstituted C₂₋₆alkynyl, substituted or unsubstituted C₁₋₆alkoxy, substituted or unsubstituted C₃₋₆carbocyclyl, or substituted or unsubstituted 3- to 10-membered heterocyclyl;
   R¹⁶ is hydrogen, halogen, —OH, —NH₂, substituted or unsubstituted C₁₋₆alkyl, substituted or unsubstituted C₂₋₆alkenyl, substituted or unsubstituted C₂₋₆alkynyl, substituted or unsubstituted C₁₋₆alkoxy, substituted or unsubstituted C₃₋₆carbocyclyl, or substituted or unsubstituted 3- to 10-membered heterocyclyl; or
   R¹⁵ and R¹⁶ are taken together with the carbon atoms to which they are attached, to form a substituted or unsubstituted C₃₋₆carbocyclyl;
   R¹⁸ is hydrogen, substituted or unsubstituted C₁₋₆alkyl, or OR^{A18}, wherein each instance of R^{A18} is independently substituted or unsubstituted C₁₋₆alkyl;
   R¹⁹ is hydrogen, substituted or unsubstituted C₁₋₆alkyl, or OR^{A19}, wherein each instance of R^{A19} is independently substituted or unsubstituted C₁₋₆alkyl;
   R^{20a} is halogen, cyano, substituted C₁₋₆alkyl, unsubstituted C₂₋₆alkyl, substituted or unsubstituted C₁₋₆alkoxy, or substituted or unsubstituted C₃₋₆carbocyclyl;
   R^{20b} is hydrogen, halogen, cyano, substituted or unsubstituted C₁₋₆alkyl, substituted or unsubstituted C₁₋₆alkoxy, or substituted or unsubstituted C₃₋₆carbocyclyl;
   or R^{20a} and R^{20b} are both simultaneously —CH₃;
   X is —(C(R^{X})₂)ₙ— or —O—, wherein R^{X} is hydrogen or fluorine, or one R^{X} group and R^{23b} are joined to form a double bond;
   n is 1, 2 or 3;
   each instance of R^{23a} and R^{23b} is independently hydrogen or fluorine;
   R^{24a} is selected from the group consisting of substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl;
   R^{24b} is hydrogen, substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, or substituted or unsubstituted 5- to 10-membered heteroaryl;
   or R^{24a} and R^{24b}, taken together with the carbon atom to which they are attached, form a substituted or unsubstituted C₃₋₆carbocyclyl or a substituted or unsubstituted 3- to 10-membered heterocyclyl;
   q is 0, 1, or 2;
   r is 1 or 2;
   s is 1, 2, or 3; and
   each [symbol] independently represents a single or double bond, provided if the bond between C5 and C6 is a double bond, R⁵ is absent and if the bond between C9 and C11 is a double bond, R⁹ and R^{11b} are absent.

In some embodiments, the compound of Formula (A-I) is selected from any one of Compounds A-1 to A-26 in Table A-1 and pharmaceutically acceptable salts thereof.

In one aspect, the disclosure provides a compound of Formula (B-I):

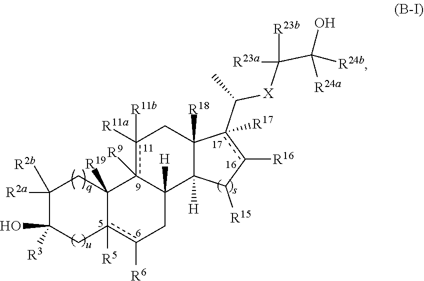

or a pharmaceutically acceptable salt thereof;
wherein:
   each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein R^{A2} is hydrogen or substituted or unsubstituted C₁₋₆alkyl;
   R³ is substituted or unsubstituted C₁₋₆alkyl, substituted or unsubstituted C₂₋₆ alkenyl, substituted or unsubstituted C₂₋₆alkynyl, or substituted or unsubstituted C₃₋₁₀carbocyclyl;
   R⁵ is absent, hydrogen, halogen, or substituted or unsubstituted C₁₋₆alkyl;
   R⁶ is hydrogen, substituted or unsubstituted C₁₋₆alkyl, or halogen;
   R⁹ is absent or hydrogen; or
   R⁹ and R^{11a} are taken together with the carbon atoms to which they are attached to form a substituted or unsubstituted C₃₋₆carbocyclyl;
   R^{11a} is hydrogen, halogen, substituted or unsubstituted C₁₋₆alkyl, or —OR^{A11}, wherein R^{A11} is hydrogen or substituted or unsubstituted C₁₋₆alkyl;
   R^{11b} is absent or hydrogen; or
   R^{11a} and R^{11b} are joined to form an oxo (═O) group;
   R¹⁵ is hydrogen, halogen, —OH, —NH₂, substituted or unsubstituted C₁₋₆alkyl, substituted or unsubstituted C₂₋₆alkenyl, substituted or unsubstituted C₂₋₆alkynyl, substituted or unsubstituted C₁₋₆alkoxy, substituted or unsubstituted C₃₋₆carbocyclyl or substituted or unsubstituted 3- to 10-membered heterocyclyl;
   R¹⁶ is hydrogen, halogen —OH, —NH₂, substituted or unsubstituted C₁₋₆alkyl, substituted or unsubstituted C₂₋₆alkenyl, substituted or unsubstituted C₂₋₆alkynyl, substituted or unsubstituted C₁₋₆alkoxy, substituted or unsubstituted C₃₋₆carbocyclyl or substituted or unsubstituted 3- to 10-membered heterocyclyl;
   R¹⁷ is absent, hydrogen or substituted or unsubstituted C₁₋₆alkyl; provided that when the bond between C16 and C17 is a double bond, then R¹⁷ is absent;
   or R¹⁵ and R¹⁶ are taken together with the carbon atoms to which they are attached, to form a substituted or unsubstituted C₃₋₆carbocyclyl;
   or R¹⁶ and R¹⁷ are taken together with the carbon atoms to which they are attached to form a substituted or unsubstituted C₃₋₆carbocyclyl;
   provided that R¹⁵ and R¹⁶ and R¹⁶ and R¹⁷ cannot both be taken together with the carbon atoms to which they are attached, to form a substituted or unsubstituted C₃₋₆carbocyclyl; and
   provided that at least one of R¹⁵, R¹⁶, and R¹⁷ is not hydrogen;
   R¹⁸ is hydrogen, substituted or unsubstituted C₁₋₆alkyl, OR^{A18}, wherein R^{A18} is substituted or unsubstituted C₁₋₆alkyl;
   R¹⁹ is hydrogen, substituted or unsubstituted C₁₋₆alkyl, OR^{A19}, wherein R^{A19} is substituted or unsubstituted C₁₋₆alkyl;
   X is —(C(R^{X})₂)ₙ— or —O—, wherein R^{X} is hydrogen or fluorine, or one R^{X} group and R^{23b} are joined to form a double bond;
   n is 1, 2 or 3;
   each instance of R^{23a} and R^{23b} is independently hydrogen or fluorine;
   R^{24a} is selected from the group consisting of substituted or unsubstituted C₁₋₆alkyl, substituted or unsubstituted C₂₋₆alkenyl, substituted or unsubstituted C₂₋₆alkynyl, substituted or unsubstituted C₃₋₁₀carbocyclyl, substituted or unsubstituted 3- to 10-membered heterocyclyl, substituted or unsubstituted C₆₋₁₄ aryl, and substituted or unsubstituted 5- to 10-membered heteroaryl;
   R^{24b} is hydrogen or substituted or unsubstituted C₁₋₆alkyl;
   or R^{24a} and R^{24b}, taken together with the carbon atom to which they are attached, form a substituted or unsubstituted C₃₋₆carbocyclyl, or substituted or unsubstituted 3- to 10-membered heterocyclyl;
   q is 0, 1 or 2;
   u is 1 or 2;
   s is 1 or 2; and
   each [symbol] independently represents a single or double bond, provided if the bond between C5 and C6 is a double bond, R⁵ is absent, and if the bond between C9 and C11 is a double bond, R⁹ and R^{11b} are absent.

In some embodiments, the compound of Formula (B-I) is selected from any one of Compounds B-1 to B-24 in Table B-1 and pharmaceutically acceptable salts thereof.

In one aspect, the disclosure provides a compound of Formula (C-I):

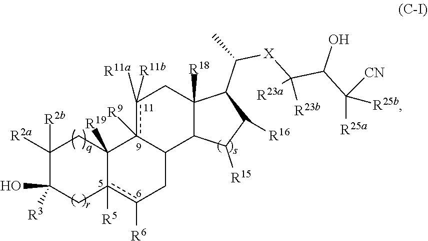

or a pharmaceutically acceptable salt thereof;
wherein:
   each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein R^{A2} is hydrogen or substituted or unsubstituted C₁₋₆alkyl;
   R³ is substituted or unsubstituted C₁₋₆alkyl, substituted or unsubstituted C₂₋₆ alkenyl, or substituted or unsubstituted C₂₋₆alkynyl, or substituted or unsubstituted C₃₋₁₀carbocyclyl;
   R⁵ is absent, hydrogen, halogen, or substituted or unsubstituted C₁₋₆alkyl;
   R⁶ is hydrogen, halogen, or substituted or unsubstituted C₁₋₆alkyl;
   R⁹ is absent or hydrogen; or
   R⁹ and R^{11a} are taken together with the carbon atoms to which they are attached to form a substituted or unsubstituted C₃₋₆carbocyclyl ring;
   R^{11a} is hydrogen, halogen, substituted or unsubstituted C₁₋₆alkyl, or —OR^{A11}, wherein R^{A11} is hydrogen or substituted or unsubstituted C₁₋₆alkyl;
   R^{11b} is absent or hydrogen; or
   R^{11a} and R^{11b} are joined to form an oxo (═O) group;
   each instance of R¹⁵ is hydrogen, halogen, —OH, —NH₂, substituted or unsubstituted C₁₋₆alkyl, substituted or unsubstituted C₂₋₆alkenyl, substituted or unsubstituted C₂₋₆alkynyl, substituted or unsubstituted C₁₋₆alkoxy, substituted or unsubstituted C₃₋₆carbocyclyl, or substituted or unsubstituted 3- to 10-membered heterocyclyl;
   R¹⁶ is hydrogen, halogen, —OH, —NH₂, substituted or unsubstituted C₁₋₆alkyl, substituted or unsubstituted C₂₋₆alkenyl, substituted or unsubstituted C₂₋₆alkynyl, substituted or unsubstituted C₁₋₆alkoxy, substituted or unsubstituted C₃₋₆carbocyclyl, or substituted or unsubstituted 3- to 10-membered heterocyclyl; or
   R¹⁵ and R¹⁶ are taken together with the carbon atoms to which they are attached, to form a substituted or unsubstituted C₃₋₆carbocyclyl;
   R¹⁸ is hydrogen, substituted or unsubstituted C₁₋₆alkyl, or OR^{A18}, wherein each instance of R^{A18} is independently substituted or unsubstituted C₁₋₆alkyl;
   R¹⁹ is hydrogen, substituted or unsubstituted C₁₋₆alkyl, or OR^{A19}, wherein each instance of R^{A19} is independently substituted or unsubstituted C₁₋₆alkyl;
   X is —(C(R^{X})₂)ₙ— or —O—, wherein R^{X} is hydrogen or fluorine, or one R^{X} group and R^{23b} are joined to form a double bond;
   n is 1, 2 or 3;
   each of R^{23a} and R^{23b} is independently hydrogen or fluorine;
   R^{25a} is hydrogen, substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, or substituted or unsubstituted 5- to 10-membered heteroaryl;
   R^{25b} is hydrogen, substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, or substituted or unsubstituted 5- to 10-membered heteroaryl;
   or R^{25a} and R^{25b}, taken together with the carbon atom to which they are attached, form a substituted or unsubstituted C₃₋₆carbocyclyl or substituted or unsubstituted 3- to 10-membered heterocyclyl;
   q is 0, 1 or 2;
   r is 1 or 2;
   s is 1 or 2; and
   each [symbol] independently represents a single or double bond;
   provided if the bond between C5 and C6 is a double bond, R⁵ is absent and if the bond between C9 and C11 is a double bond, R⁹ and R^{11b} are absent.

In some embodiments, the compound of Formula (C-I) is selected from any one of Compounds C-1 to C-35 in Table C-1 and pharmaceutically acceptable salts thereof.

In one aspect, the disclosure provides a compound of Formula (D-I):

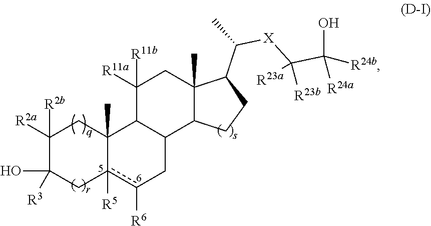

or a pharmaceutically acceptable salt thereof;
wherein:
   each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein R^{A2} is hydrogen or substituted or unsubstituted C₁₋₆alkyl;
   R³ is substituted or unsubstituted C₁₋₆alkyl, substituted or unsubstituted C₂₋₆ alkenyl, or substituted or unsubstituted C₂₋₆alkynyl, or substituted or unsubstituted C₃₋₁₀ carbocyclyl;
   R⁵ is absent, hydrogen, halogen, or substituted or unsubstituted C₁₋₆alkyl;
   R⁶ is hydrogen, substituted or unsubstituted C₁₋₆alkyl, or halogen;
   R^{11a} is hydrogen or —OR^{A11}, wherein R^{A11} is hydrogen or substituted or unsubstituted C₁₋₆alkyl, and R^{11b} is hydrogen; or R^{11a} and R^{11b} are joined to form an oxo (═O) group;
   X is —(C(R^{X})₂)ₙ— or —O—, wherein R^{X} is hydrogen or fluorine, or one R^{X} group and R^{23b} are joined to form a double bond;
   n is 1, 2 or 3;
   each of R^{23a} and R^{23b} is independently hydrogen or fluorine;
   R^{24a} is selected from the group consisting of substituted or unsubstituted C₁₋₆alkyl, substituted or unsubstituted C26 alkenyl, substituted or unsubstituted C₂₋₆ alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl group; and
   R^{24b} is hydrogen or a substituted or unsubstituted C₁₋₆alkyl;
   or R^{24a} and R^{24b}, taken together with the carbon atom to which they are attached, form a substituted or unsubstituted C₃₋₆carbocyclyl, or substituted or unsubstituted 3- to 10-membered heterocyclyl;
   q is 0, 1 or 2;
   r is 1 or 2;
   s is 1, 2, 3 or 4;
   provided that a) q and r are not both 1, or b) when q is 0, r is not 2; and
   [symbol] represents a single or double bond; provided if the bond between C5 and C6 is a double bond, R⁵ is absent.

In some embodiments, the compound of Formula (D-I) is selected from any one of Compounds D-1 to D-17 in Table D-1 and pharmaceutically acceptable salts thereof.

In one aspect, the disclosure provides a compound of Formula (E-I):

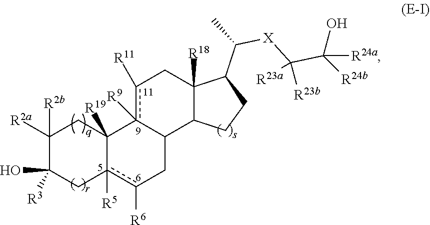

or a pharmaceutically acceptable salt thereof;
wherein:
   each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein R^{A2} is hydrogen or substituted or unsubstituted C₁₋₆alkyl;
   R³ is substituted or unsubstituted C₁₋₆alkyl, substituted or unsubstituted C₂₋₆alkenyl, or substituted or unsubstituted C₂₋₆alkynyl, or substituted or unsubstituted C₃₋₁₀carbocyclyl;
   [symbol] represents a single or double bond;
   when the bond between C9 and C11 is a double bond, R⁹ is absent and R¹¹ is selected from the group consisting of hydrogen, halogen, substituted or unsubstituted C₁₋₆alkyl, substituted or unsubstituted C₂₋₆alkenyl, substituted or unsubstituted C₂₋₆alkynyl, substituted or unsubstituted C₃₋₁₀carbocyclyl, substituted or unsubstituted C₁₋₆alkoxy, substituted or unsubstituted 3- to 10-membered heterocyclyl, —OH, and -amino;
   when the bond between C9 and C11 is a single bond, then R⁹ is hydrogen and R¹¹ is halogen or substituted or unsubstituted C₁₋₆alkyl; or R⁹ and R¹¹, taken together with the carbon atoms to which they are attached, form a substituted or unsubstituted C₃₋₆carbocyclyl ring;
   when the bond between C5 and C6 is a double bond, then R⁵ is absent and R⁶ is hydrogen, halogen, or substituted or unsubstituted C₁₋₆alkyl;
   when the bond between C5 and C6 is a single bond, then each of R⁵ and R⁶ is independently hydrogen, halogen, or substituted or unsubstituted C₁₋₆alkyl;
   R¹⁸ is hydrogen, substituted or unsubstituted C₁₋₆alkyl, or OR^{A18}, wherein R^{A18} is substituted or unsubstituted C₁₋₆alkyl;
   R¹⁹ is hydrogen, substituted or unsubstituted C₁₋₆alkyl, or OR^{A19}, wherein R^{A19} is substituted or unsubstituted C₁₋₆alkyl;
   X is —(C(R^{X})₂)ₙ— or —O—, wherein R^{X} is hydrogen or fluorine, or one R^{X} group and R^{23b} are joined to form a double bond;
   n is 1 or 2;
   each of R^{23a} and R^{23b} is independently hydrogen or fluorine;
   R^{24a} is selected from the group consisting of substituted or unsubstituted C₁₋₆ alkyl, substituted or unsubstituted C₂₋₆alkenyl, substituted or unsubstituted C₂₋₆ alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl;
   R^{24b} is hydrogen or a substituted or unsubstituted C₁₋₆alkyl; or
   R^{24a} and R^{24b}, taken together with the carbon atoms to which they are attached, form a substituted or unsubstituted C₃₋₆carbocyclyl, or substituted or unsubstituted 3- to 6-membered heterocyclyl;
   q is 0, 1 or 2;
   r is 1 or 2; and
   s is 1 or 2.

In some embodiments, the compound of Formula (E-I) is selected from any one of Compounds E-1 to E-16 and C-26 to C-35 in Table E-1 and pharmaceutically acceptable salts thereof.

In one aspect, the disclosure provides a compound of Formula (F-I):

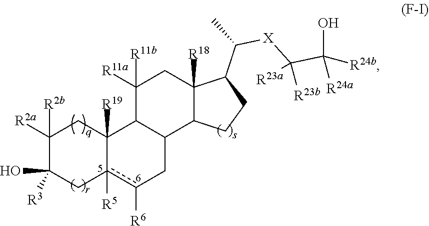

or a pharmaceutically acceptable salt thereof;
wherein:
   each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein R^{A2} is hydrogen or substituted or unsubstituted C₁₋₆alkyl;
   R³ is substituted or unsubstituted C₁₋₆alkyl, substituted or unsubstituted C₂₋₆alkenyl, substituted or unsubstituted C₂₋₆alkynyl, or substituted or unsubstituted C₃₋₁₀carbocyclyl;
   R⁵ is absent, hydrogen, substituted or unsubstituted C₁₋₆alkyl, or halogen;
   R⁶ is hydrogen, substituted or unsubstituted C₁₋₆alkyl, or halogen;
   R^{11a} is hydrogen or —OR^{A11}, wherein R^{A11} is hydrogen or substituted or unsubstituted C₁₋₆alkyl, and R^{11b} is hydrogen; or R^{11a} and R^{11b} are joined to form an oxo (═O) group;
   R¹⁸ is hydrogen, unsubstituted C₂₋₆alkyl, substituted C₁₋₆alkyl, or OR^{A18}, wherein each instance of R^{A18} is independently substituted or unsubstituted C₁₋₆alkyl;
   R¹⁹ is hydrogen, substituted or unsubstituted C₁₋₆alkyl, or OR^{A19}, each instance of R^{A19} is independently substituted or unsubstituted C₁₋₆alkyl;
   X is —(C(R^{X})₂)ₙ— or —O—, wherein R^{X} is hydrogen or fluorine, or one R^{X} group and R^{23b} are joined to form a double bond;
   n is 1, 2 or 3;
   each of R^{23a} and R^{23b} is independently hydrogen or fluorine;
   R^{24a} is selected from the group consisting of substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl; and
   R^{24b} is hydrogen or substituted or unsubstituted C₁₋₆alkyl;
   or R^{24a} and R^{24b}, taken together with the carbon atom to which they are attached, form a substituted or unsubstituted C₃₋₆carbocyclyl, or a substituted or unsubstituted 3- to 10-membered heterocyclyl;
   q is 0, 1 or 2;
   r is 1 or 2;
   s is 1 or 2; and
   [symbol] represents a single or double bond, provided if a double bond is present between C5 and C6, R⁵ is absent.

In some embodiments, the compound of Formula (F-I) is selected from any one of Compounds F-1 to F-14 in Table F-1 and pharmaceutically acceptable salts thereof.

In one aspect, the disclosure provides a compound of Formula (G-I):

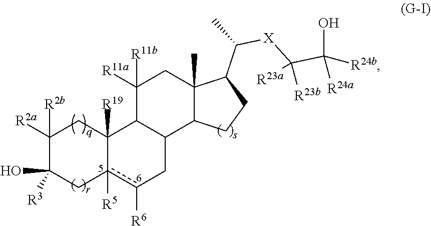

or a pharmaceutically acceptable salt thereof;
wherein:
   each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein R^{A2} is hydrogen or substituted or unsubstituted C₁₋₆alkyl;
   R³ is substituted or unsubstituted C₁₋₆alkyl, substituted or unsubstituted C₂₋₆ alkenyl, or substituted or unsubstituted C₂₋₆alkynyl;
   R⁵ is absent, hydrogen, substituted or unsubstituted C₁₋₆alkyl, or halogen;
   R⁶ is hydrogen, substituted or unsubstituted C₁₋₆alkyl, or halogen;
   R¹⁹ is hydrogen, substituted or unsubstituted C₂₋₆alkyl, or OR^{A19}; wherein each instance of R^{A19} is independently substituted or unsubstituted C₁₋₆alkyl;
   R^{11a} is hydrogen or —OR^{A11}, wherein R^{A11} is hydrogen or substituted or unsubstituted C₁₋₆alkyl, and R^{11b} is hydrogen; or R^{11a} and R^{11b} are joined to form an oxo (═O) group;
   X is —(C(R^{X})₂)ₙ— or —O—, wherein R^{X} is hydrogen or fluorine, or one R^{X} group and R^{23b} are joined to form a double bond;
   n is 1, 2 or 3;
   each of R^{23a} and R^{23b} is independently hydrogen or fluorine;
   R^{24a} is selected from the group consisting of substituted or unsubstituted C₁₋₆alkyl, substituted or unsubstituted C₂₋₆alkenyl, substituted or unsubstituted C₂₋₆ alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl; and
   R^{24b} is hydrogen or substituted or unsubstituted C₁₋₆alkyl;
   or R^{24a} and R^{24b}, taken together with the carbon atom to which they are attached, form a substituted or unsubstituted C₃₋₆carbocyclyl;
   q is 0, 1 or 2;
   r is 1 or 2;
   s is 1, 2, 3 or 4; and
   [symbol] represents a single or double bond; provided if the bond between C5 and C6 is a double bond, R⁵ is absent.

In some embodiments, the compound of Formula (G-I) is selected from any one of Compounds G-1 to G-5 in Table G-1 and pharmaceutically acceptable salts thereof.

In one aspect, the disclosure provides a compound of Formula (H-I):

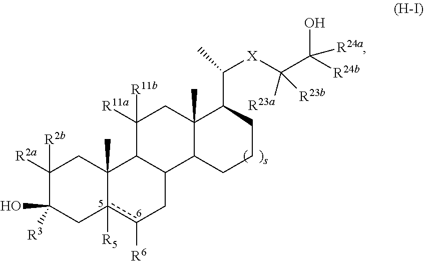

or a pharmaceutically acceptable salt thereof;
wherein:
   each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein R^{A2} is hydrogen or substituted or unsubstituted C₁₋₆alkyl;
   R³ is substituted or unsubstituted C₁₋₆alkyl, substituted or unsubstituted C₂₋₆alkenyl, substituted or unsubstituted C₂₋₆alkynyl, or substituted or unsubstituted C₃₋₁₀carbocyclyl;
   R⁵ is absent, hydrogen, halogen, or substituted or unsubstituted C₁₋₆alkyl
   R⁶ is hydrogen, substituted or unsubstituted C₁₋₆alkyl, or halogen;
   R^{11a} is hydrogen or —OR^{A11}, wherein R^{A11} is hydrogen or substituted or unsubstituted alkyl, and R^{11b} is hydrogen; or R^{11a} and R^{11b} are joined to form an oxo (═O) group;
   X is —(C(R^{X})₂)ₙ— or —O—, wherein R^{X} is hydrogen or fluorine, or one R^{X} group and R^{23b} are joined to form a double bond;
   n is 1, 2 or 3;
   each of R^{23a} and R^{23b} is independently hydrogen or fluorine;
   R^{24a} is selected from the group consisting of substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, and substituted or unsubstituted heteroaryl group; and
   R^{24b} is hydrogen or substituted or unsubstituted alkyl;
   or R^{24a} and R^{24b}, taken together with the carbon atom to which they are attached, form a substituted or unsubstituted C₃₋₆carbocyclyl or substituted or unsubstituted 3- to 10-membered heterocyclyl;
s is 1, 2 or 3; and
[symbol] represents a single or double bond, provided if the bond between C5 and C6 is a double bond, R⁵ is absent.

In some embodiments, the compound of Formula (H-I) is selected from any one of Compounds H-1 to H-11 in Table H-1 and pharmaceutically acceptable salts thereof.

In one aspect, the disclosure provides a pharmaceutical composition comprising a compound or pharmaceutically acceptable salt thereof according to the disclosure, and a pharmaceutically acceptable carrier.

In one aspect, the disclosure provides a method for treating a CNS-related condition in a subject, comprising administering to the subject an effective amount of a compound or pharmaceutically acceptable salt thereof according to the disclosure, or a pharmaceutical composition according to the disclosure.

In one aspect, the disclosure provides a method of inducing sedation or anesthesia in a subject, comprising administering to the subject an effective amount of a compound or pharmaceutically acceptable salt thereof according to the disclosure, or a pharmaceutical composition according to the disclosure.

In one aspect, the disclosure provides a compound or pharmaceutically acceptable salt thereof according to the disclosure, or a pharmaceutical composition according to the disclosure, for use in treating a CNS-related condition in a subject.

In one aspect, the disclosure provides a compound or pharmaceutically acceptable salt thereof according to the disclosure, or a pharmaceutical composition according to the disclosure, for use in inducing sedation or anesthesia in a subject.

In one aspect, the disclosure provides a use of a compound or pharmaceutically acceptable salt thereof according to the disclosure, or a pharmaceutical composition according to the disclosure, for the manufacture of a medicament for treating a CNS-related condition in a subject.

In one aspect, the disclosure provides a use of a compound or pharmaceutically acceptable salt thereof according to the disclosure, or a pharmaceutical composition according to the disclosure, for the manufacture of a medicament for inducing sedation or anesthesia in a subject.

In some embodiments, the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease), and tinnitus.

### DETAILED DESCRIPTION

The present disclosure provides compounds that are NMDA receptor positive allosteric modulators. The compounds of the disclosure are useful as therapeutic agents for treating, for example, CNS-related conditions including, but not limited to, adjustment disorders, anxiety disorders, cognitive disorders, dissociative disorders, eating disorders, mood disorders, schizophrenia or other psychotic disorders, sleep disorders, substance-related disorders, personality disorders, autism spectrum disorders, neurodevelopmental disorders, pain, encephalopathy secondary to a medical condition, seizure disorders, stroke, traumatic brain injury, movement disorders and tinnitus.

### General Definitions

The term “herein” means the entire application.

Unless otherwise defined herein, scientific and technical terms used in this application shall have the meanings that are commonly understood by those of ordinary skill in the art to which this disclosure belongs. Generally, nomenclature used in connection with the compounds, composition and methods described herein, are those well-known and commonly used in the art.

It should be understood that any of the embodiments described herein, including those described under different aspects of the disclosure and different parts of the specification (including embodiments described only in the Examples) can be combined with one or more other embodiments of the disclosure, unless explicitly disclaimed or improper. Combination of embodiments are not limited to those specific combinations claimed via the multiple dependent claims. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

Throughout this specification, the word “comprise” or variations such as “comprises” or “comprising”, which is synonymous with “including,” “containing,” or “characterized by,” will be understood to imply the inclusion of a stated integer (or component, element, or method) or group of integers (or components, elements, or methods), but not the exclusion of any other integer (or component, element or method) or group of integers (or components, elements, or methods).

Throughout the specification, where compositions are described as having, including, or comprising (or variations thereof), specific components, it is contemplated that compositions also may consist essentially of, or consist of, the recited components. Similarly, where methods or processes are described as having, including, or comprising specific process steps, the processes also may consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

The term “including,” as used herein, means “including but not limited to.” “Including” and “including but not limited to” are used interchangeably. Thus, these terms will be understood to imply the inclusion of a stated integer (or component, element or method) or group of integers (or components, elements or methods), but not the exclusion of any other integer (or component, element or method) or group of integers (or components, elements or methods).

As used herein, “about” or “approximately” means within an acceptable error range for the particular value as determined by one of ordinary skill in the art, which will depend in part on how the value is measured or determined, i.e., the limitations of the measurement system.

The use of the terms “a” and “an” and “the” and similar referents in the context of describing the elements (especially in the context of the following claims) are to be construed to cover both the singular and the plural, unless otherwise indicated herein or clearly contradicted by context.

The term “or” as used herein should be understood to mean “and/or,” unless the context clearly indicates otherwise.

Recitation of ranges of values herein are merely intended to serve as a shorthand method of referring individually to each separate value falling within the range and including the endpoints, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

All of the publications, patents and published patent applications referred to in this application are specifically incorporated by reference herein. In case of conflict, the present specification, including its specific definitions, will control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers, e.g., stereoisomers, can be isolated from mixtures by methods known to those skilled in the art, including chiral high-performance liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The disclosure additionally encompasses compounds described herein as individual isomers substantially free of other isomers, and alternatively, as mixtures of various isomers.

Compounds that have the same molecular formula but differ in the nature or sequence of bonding of their atoms or the arrangement of their atoms in space are termed “isomers.” Isomers that differ in the arrangement of their atoms in space are termed “stereoisomers.” Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture.”

As used herein, a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of all enantiomers or stereoisomers of the compound. As used herein, the term “diastereomeric purity” refers to the amount of a compound having the depicted absolute stereochemistry, expressed as a percentage of the total amount of the depicted compound and its diastereomers. The term “diastereomerically pure” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the diastereomer. Methods for determining diastereomeric and enantiomeric purity are well-known in the art. Diastereomeric purity can be determined by any analytical method capable of quantitatively distinguishing between a compound and its diastereomers, such as high-performance liquid chromatography (HPLC).

In the compositions provided herein, an enantiomerically pure compound can be present with other active or inactive ingredients. For example, a pharmaceutical composition comprising enantiomerically pure R-position/center/carbon compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S compound, by total weight of the compound. For example, a pharmaceutical composition comprising enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound. In certain embodiments, the active ingredient can be formulated with little or no excipient or carrier.

As used herein, the term “diastereomeric purity” refers to the amount of a compound having the depicted absolute stereochemistry, expressed as a percentage of the total amount of the depicted compound and its diastereomers. The term “diastereomerically pure” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the diastereomer. Methods for determining diastereomeric and enantiomeric purity are well-known in the art. Diastereomeric purity can be determined by any analytical method capable of quantitatively distinguishing between a compound and its diastereomers, such as high-performance liquid chromatography (HPLC).

Compound described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; and the like.

When a range of values is listed, it is intended to encompass each value and sub-range within the range. For example “C₁₋₆alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The following terms are intended to have the meanings presented therewith below and are useful in understanding the description and intended scope of the present disclosure. It should also be understood that when described herein any of the moieties defined herein may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein.

“Aliphatic” refers to an alkyl, alkenyl, alkynyl, or carbocyclyl group, as defined herein.

“Alkyl” refers to a radical of a straight-chain or branched saturated hydrocarbon group having from 1 to 20 carbon atoms (“C₁₋₂₀ alkyl”). In some embodiments, an alkyl group has 1 to 6 carbon atoms (“C₁₋₆alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). Examples of C₁₋₆alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Unless otherwise specified, each instance of an alkyl group is independently optionally substituted, i.e., unsubstituted (an “unsubstituted alkyl”) or substituted (a “substituted alkyl”) with one or more substituents; e.g., for instance from 1 to 4 substituents, 1 to 3 substituents, or 1 substituent. Common alkyl abbreviations include Me (—CH₃), Et (—CH₂CH₃), iPr (—CH(CH₃)₂), nPr (—CH₂CH₂CH₃), n-Bu (—CH₂CH₂CH₂CH₃), or i-Bu (—CH₂CH(CH₃)₂).

As used herein, “alkylene,” “alkenylene,” “alkynylene,” “heteroalkylene,” “heteroalkenylene,” and “heteroalkynylene,” refer to a divalent radical of an alkyl, alkenyl, alkynyl, heteroalkyl, heteroalkenyl, and heteroalkynyl group, respectively. When a range or number of carbons is provided for a particular “alkylene,” “alkenylene,” “alkynylene,” “heteroalkylene,” “heteroalkenylene,” or “heteroalkynylene,” group, it is understood that the range or number refers to the range or number of carbons in the linear carbon divalent chain. “Alkylene,” “alkenylene,” “alkynylene,” “heteroalkylene,” “heteroalkenylene,” and “heteroalkynylene” groups may be substituted or unsubstituted with one or more substituents as described herein.

“Alkylene” refers to an alkyl group wherein two hydrogens are removed to provide a divalent radical, and which may be substituted or unsubstituted. Unsubstituted alkylene groups include, but are not limited to, methylene (—CH₂—), ethylene (—CH₂CH₂—), propylene (—CH₂CH₂CH₂—), butylene (—CH₂CH₂CH₂CH₂—), pentylene (—CH₂CH₂CH₂CH₂CH₂—), hexylene (—CH₂CH₂CH₂CH₂CH₂CH₂—), and the like. Exemplary substituted alkylene groups, e.g., substituted with one or more halo, —NO₂, —OH, C₁-C₆ alkoxy, C₁-C₆ alkyl (e.g., methyl) groups, including but not limited to, substituted methylene (—CH(CH₃)—, (—C(CH₃)₂—), substituted ethylene (—CH(CH₃) CH₂—, —CH₂CH(CH₃)—, —C(CH₃)₂CH₂—, —CH₂C(CH₃)₂—), substituted propylene (—CH(CH₃) CH₂CH₂—, —CH₂CH(CH₃) CH₂—, —CH₂CH₂CH(CH₃)—, —C(CH₃)₂CH₂CH₂—, —CH₂C(CH₃)₂CH₂—, —CH₂CH₂C(CH₃)₂—), or C₁-C₆ cycloalkyl, and the like. Alkylene abbreviations include, but are not limited to, —(CH(CH₃))—, —(CH(CH₂CH₃))—, —(CH(CH₂CH₂CH₃))—, —(CH(CH₂CH₂CH₂CH₃))—, —(CH₂CH(CH₂CH₂CH₂CH₃))—, —(CH₂CH₂CH(CH₂CH₂CH₂CH₃))—, —(CH(CH₃) CH₂)—, —(CH(CH₃) CH₂CH₂)—, —(CH(CH₃) CH₂CH₂CH₂)—, —(CH₂CH(CH₃) CH₂)—, —(CH₂CH(CH₃) CH₂CH₂)—, and —(CH₂CH₂CH(CH₃) CH₂CH₂)—.

“Alkenyl” refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C₂₋₂₀ alkenyl”). In certain embodiments, alkenyl does not contain any triple bonds. In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₂₋₁₀ alkenyl. In certain embodiments, the alkenyl group is substituted C₂₋₁₀ alkenyl.

“Alkenylene” refers to an alkenyl group wherein two hydrogens are removed to provide a divalent radical, and which may be substituted or unsubstituted. Exemplary unsubstituted divalent alkenylene groups include, but are not limited to, ethenylene (—CH═CH—) and propenylene (e.g., —CH═CHCH₂—, —CH₂—CH═CH—). Exemplary substituted alkenylene groups, e.g., substituted with one or more alkyl (methyl) groups, include but are not limited to, substituted ethylene (—C(CH₃)═CH—, —CH═C(CH₃)—), substituted propylene (e.g., —C(CH₃)—CHCH₂—, —CH═C(CH₃) CH₂—, —CH═CHCH(CH₃)—, —CH═CHC(CH₃)₂—, —CH(CH₃)—CH═CH—, —C(CH₃)₂—CH═CH—, —CH₂—C(CH₃)—CH—, —CH₂—CH═C(CH₃)—), and the like.

“Alkynyl” refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds) (“C₂₋₂₀ alkynyl”). In certain embodiments, alkynyl does not contain any double bonds. In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆alkenyl groups include the aforementioned C₂₋₄ alkynyl groups as well as pentynyl (C₅), hexynyl (C₆), and the like. Additional examples of alkynyl include heptynyl (C₈), octynyl (C₈), and the like. Unless otherwise specified, each instance of an alkynyl group is independently optionally substituted, i.e., unsubstituted (an “unsubstituted alkynyl”) or substituted (a “substituted alkynyl”) with one or more substituents; e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₁₀ alkynyl.

“Alkynylene” refers to a linear alkynyl group wherein two hydrogens are removed to provide a divalent radical, and which may be substituted or unsubstituted. Exemplary divalent alkynylene groups include, but are not limited to, substituted or unsubstituted ethynylene, substituted or unsubstituted propynylene, and the like.

The term “heteroalkyl,” as used herein, refers to an alkyl group, as defined herein, which further comprises 1 or more (e.g., 1, 2, 3, or 4) heteroatoms (e.g., oxygen, sulfur, nitrogen, boron, silicon, phosphorus) within the parent chain, wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkyl group refers to a saturated group having from 1 to 10 carbon atoms and 1, 2, 3, or 4 heteroatoms (“heteroC₁₋₁₀ alkyl”). In some embodiments, a heteroalkyl group is a saturated group having 1 to 9 carbon atoms and 1, 2, 3, or 4 heteroatoms (“heteroC₁₋₉ alkyl”). In some embodiments, a heteroalkyl group is a saturated group having 1 to 8 carbon atoms and 1, 2, 3, or 4 heteroatoms (“heteroC₁₋₈ alkyl”). In some embodiments, a heteroalkyl group is a saturated group having 1 to 7 carbon atoms and 1, 2, 3, or 4 heteroatoms (“heteroC₁₋₇ alkyl”). In some embodiments, a heteroalkyl group is a group having 1 to 6 carbon atoms and 1, 2, or 3 heteroatoms (“heteroC₁₋₆alkyl”). In some embodiments, a heteroalkyl group is a saturated group having 1 to 5 carbon atoms and 1 or 2 heteroatoms (“heteroC₁₋₅ alkyl”). In some embodiments, a heteroalkyl group is a saturated group having 1 to 4 carbon atoms and 1 or 2 heteroatoms (“heteroC₁₋₄ alkyl”). In some embodiments, a heteroalkyl group is a saturated group having 1 to 3 carbon atoms and 1 heteroatom (“heteroC₁₋₃ alkyl”). In some embodiments, a heteroalkyl group is a saturated group having 1 to 2 carbon atoms and 1 heteroatom (“heteroCC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms (“heteroC₂₋₆alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₀ alkyl. In certain embodiments, the heteroalkyl group is a substituted heteroC₁₋₁₀ alkyl.

The term “heteroalkenyl,” as used herein, refers to an alkenyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkenyl group refers to a group having from 2 to 10 carbon atoms, at least one double bond, and 1, 2, 3, or 4 heteroatoms (“heteroC₂₋₁₀ alkenyl”). In some embodiments, a heteroalkenyl group has 2 to 9 carbon atoms at least one double bond, and 1, 2, 3, or 4 heteroatoms (“heteroC₂₋₉ alkenyl”). In some embodiments, a heteroalkenyl group has 2 to 8 carbon atoms, at least one double bond, and 1, 2, 3, or 4 heteroatoms (“heteroC₂₋₈ alkenyl”). In some embodiments, a heteroalkenyl group has 2 to 7 carbon atoms, at least one double bond, and 1, 2, 3, or 4 heteroatoms (“heteroC₂₋₇ alkenyl”). In some embodiments, a heteroalkenyl group has 2 to 6 carbon atoms, at least one double bond, and 1, 2, or 3 heteroatoms (“heteroC₂₋₆alkenyl”). In some embodiments, a heteroalkenyl group has 2 to 5 carbon atoms, at least one double bond, and 1 or 2 heteroatoms (“heteroC₂₋₅ alkenyl”). In some embodiments, a heteroalkenyl group has 2 to 4 carbon atoms, at least one double bond, and 1 or 2 heteroatoms (“heteroC₂₋₄ alkenyl”). In some embodiments, a heteroalkenyl group has 2 to 3 carbon atoms, at least one double bond, and 1 heteroatom (“heteroC₂₋₃alkenyl”). In some embodiments, a heteroalkenyl group has 2 to 6 carbon atoms, at least one double bond, and 1 or 2 heteroatoms (“heteroC₂₋₆alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₁₀ alkenyl. In certain embodiments, the heteroalkenyl group is a substituted heteroC₂₋₁₀ alkenyl.

The term “heteroalkynyl,” as used herein, refers to an alkynyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms is inserted between a carbon atom and the parent molecule, i.e., between the point of attachment. In certain embodiments, a heteroalkynyl group refers to a group having from 2 to 10 carbon atoms, at least one triple bond, and 1, 2, 3, or 4 heteroatoms (“heteroC₂₋₁₀ alkynyl”). In some embodiments, a heteroalkynyl group has 2 to 9 carbon atoms, at least one triple bond, and 1, 2, 3, or 4 heteroatoms (“heteroC₂₋₉ alkynyl”). In some embodiments, a heteroalkynyl group has 2 to 8 carbon atoms, at least one triple bond, and 1, 2, 3, or 4 heteroatoms (“heteroC₂₋₈ alkynyl”). In some embodiments, a heteroalkynyl group has 2 to 7 carbon atoms, at least one triple bond, and 1, 2, 3, or 4 heteroatoms (“heteroC₂₋₇ alkynyl”). In some embodiments, a heteroalkynyl group has 2 to 6 carbon atoms, at least one triple bond, and 1, 2, or 3 heteroatoms (“heteroC₂₋₆alkynyl”). In some embodiments, a heteroalkynyl group has 2 to 5 carbon atoms, at least one triple bond, and 1 or 2 heteroatoms (“heteroC₂₋₅ alkynyl”). In some embodiments, a heteroalkynyl group has 2 to 4 carbon atoms, at least one triple bond, and 1 or 2 heteroatoms (“heteroC₂₋₄ alkynyl”). In some embodiments, a heteroalkynyl group has 2 to 3 carbon atoms, at least one triple bond, and 1 heteroatom (“heteroC₂₋₃ alkynyl”). In some embodiments, a heteroalkynyl group has 2 to 6 carbon atoms, at least one triple bond, and 1 or 2 heteroatoms (“heteroC₂₋₆alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₁₀ alkynyl. In certain embodiments, the heteroalkynyl group is a substituted heteroC₂₋₁₀ alkynyl.

“Aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). Aryl” also includes ring systems wherein the aryl ring, as defined herein, is fused with one or more carbocyclyl or heterocyclyl groups wherein the radical or point of attachment is on the aryl ring, and in such instances, the number of carbon atoms continue to designate the number of carbon atoms in the aryl ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, and trinaphthalene. Particularly aryl groups include phenyl, naphthyl, indenyl, and tetrahydronaphthyl. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

In certain embodiments, an aryl group substituted with one or more of groups selected from halo, C₁-C₈ alkyl, C₁-C₈ haloalkyl, cyano, hydroxy, C₁-C₈ alkoxy, and amino.

Examples of representative substituted aryls include the following

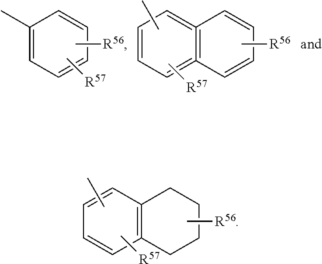

wherein one of R⁵⁶ and R⁵⁷ may be hydrogen and at least one of R⁵⁶ and R⁵⁷ is each independently selected from C₁-C₈ alkyl, C₁-C₈ haloalkyl, 4-10 membered heterocyclyl, alkanoyl, C₁-C₈ alkoxy, heteroaryloxy, alkylamino, arylamino, heteroarylamino, NR⁵⁸COR⁵⁹, NR⁵⁸SOR⁵⁹NR⁵⁸SO₂R⁵⁹, COOalkyl, COOaryl, CONR⁵⁸R⁵⁹, CONR⁵⁸OR⁵⁹, NR⁵⁸R⁵⁹, SO₂NR⁵⁸R⁵⁹, S-alkyl, SOalkyl, SO₂alkyl, Saryl, SOaryl, SOzaryl; or R⁵⁶ and R⁵⁷ may be joined to form a cyclic ring (saturated or unsaturated) from 5 to 8 atoms, optionally containing one or more heteroatoms selected from the group N, O, or S. R⁶⁰ and R⁶¹ are independently hydrogen, C₁-C₈ alkyl, C₁-C₄ haloalkyl, C₃-C₁₀ cycloalkyl, 4-10 membered heterocyclyl, C₆-C₁₀ aryl, substituted C₆-C₁₀ aryl, 5-10 membered heteroaryl, or substituted 5-10 membered heteroaryl.

“Fused aryl” refers to an aryl having two of its ring carbon in common with a second aryl or heteroaryl ring or with a carbocyclyl or heterocyclyl ring.

“Aralkyl” is a subset of alkyl and aryl, as defined herein, and refers to an optionally substituted alkyl group substituted by an optionally substituted aryl group.

“Heteroaryl” refers to a radical of a 5-10 membered monocyclic or bicyclic 4n+2 aromatic ring system (e.g., having 6 or 10 electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively.

Examples of representative heteroaryls include the following:

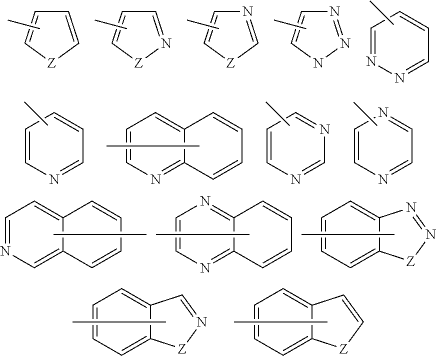

wherein each Z is selected from carbonyl, N, NR⁶⁵, O, and S; and R⁶⁵ is independently hydrogen, C₁-C₈ alkyl, C₃-C₁₀ cycloalkyl, 4-10 membered heterocyclyl, C₆-C₁₀ aryl, and 5-10 membered heteroaryl.

“Heteroaralkyl” is a subset of alkyl and heteroaryl, as defined herein, and refers to an optionally substituted alkyl group substituted by an optionally substituted heteroaryl group.

“Carbocyclyl” or “carbocyclic” refers to a radical of a non-aromatic cyclic hydrocarbon group having from 3 to 10 ring carbon atoms (“C₃₋₁₀carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆carbocyclyl”). In some embodiments, a carbocyclyl group has 3 to 6 ring carbon atoms (“C₃₋₆carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆carbocyclyl groups as well as cycloheptyl (C₈), cycloheptenyl (C₈), cycloheptadienyl (C₇), cycloheptatrienyl (C₇), cyclooctyl (C₈), cyclooctenyl (C₈), bicyclo[2.2.1]heptanyl (C₈), bicyclo[2.2.2]octanyl (C₈), and the like. Exemplary C₃₋₁₀carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In certain embodiments, the carbocyclyl group is unsubstituted C₃₋₁₀carbocyclyl. In certain embodiments, the carbocyclyl group is a substituted C₃₋₁₀carbocyclyl.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

“Heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C6 aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Hetero” when used to describe a compound or a group present on a compound means that one or more carbon atoms in the compound or group have been replaced by a nitrogen, oxygen, or sulfur heteroatom. Hetero may be applied to any of the hydrocarbyl groups described above such as alkyl, e.g., heteroalkyl, cycloalkyl, e.g., heterocyclyl, aryl, e.g. heteroaryl, cycloalkenyl, e.g. cycloheteroalkenyl, and the like having from 1 to 5, and particularly from 1 to 3 heteroatoms.

“Acyl” refers to a radical —C(O)R²⁰, where R²⁰ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl, as defined herein. “Alkanoyl” is an acyl group wherein R²⁰ is a group other than hydrogen. Representative acyl groups include, but are not limited to, formyl (—CHO), acetyl (—C(═O)CH₃), cyclohexylcarbonyl, cyclohexylmethylcarbonyl, benzoyl (—C(═O)Ph), benzylcarbonyl (—C(═O) CH₂Ph), —C(O)—C₁-C₈ alkyl, —C(O)—(CH₂)ₜ(C₆-C₁₀ aryl), —C(O)—(CH₂)ₜ(5-10 membered heteroaryl), —C(O)—(CH₂)ₜ(C₃-C₁₀ cycloalkyl), and —C(O)—(CH₂)ₜ(4-10 membered heterocyclyl), wherein t is an integer from 0 to 4. In certain embodiments, R²⁰ is C₁-C₈ alkyl, substituted with halo or hydroxy; or C₃-C₁₀ cycloalkyl, 4-10 membered heterocyclyl, C₆-C₁₀ aryl, arylalkyl, 5-10 membered heteroaryl or heteroarylalkyl, each of which is substituted with unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy.

“Alkoxy” refers to the group —OR²⁹ where R²⁹ is substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, or substituted or unsubstituted heteroaryl. Particular alkoxy groups are methoxy, ethoxy, n-propoxy, isopropoxy, n-butoxy, tert-butoxy, sec-butoxy, n-pentoxy, n-hexoxy, and 1,2-dimethylbutoxy. Particular alkoxy groups are lower alkoxy, i.e. with between 1 and 6 carbon atoms. Further particular alkoxy groups have between 1 and 4 carbon atoms.

In certain embodiments, R²⁹ is a group that has 1 or more substituents, for instance from 1 to 5 substituents, and particularly from 1 to 3 substituents, in particular 1 substituent, selected from the group consisting of amino, substituted amino, C₆-C₁₀ aryl, aryloxy, carboxyl, cyano, C₃-C₁₀ cycloalkyl, 4-10 membered heterocyclyl, halogen, 5-10 membered heteroaryl, hydroxyl, nitro, thioalkoxy, thioaryloxy, thiol, alkyl-S(O)—, aryl-S(O)—, alkyl-S(O)₂— and aryl-S(O)₂—. Exemplary ‘substituted alkoxy’ groups include, but are not limited to, —O—(CH₂)ₜ(C₆-C₁₀ aryl), —O—(CH₂)ₜ(5-10 membered heteroaryl), —O—(CH₂)ₜ(C₃-C₁₀ cycloalkyl), and —O—(CH₂)ₜ(4-10 membered heterocyclyl), wherein t is an integer from 0 to 4 and any aryl, heteroaryl, cycloalkyl or heterocyclyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. Particular exemplary ‘substituted alkoxy’ groups are —OCF₃, —OCH₂CF₃, —OCH₂Ph, —OCH₂-cyclopropyl, —OCH₂CH₂OH, and —OCH₂CH₂NMe₂.

“Amino” refers to the radical —NH₂.

“Substituted amino” refers to an amino group of the formula —N(R³⁸)₂ wherein R³⁸ is hydrogen, substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, substituted or unsubstituted heteroaryl, or an amino protecting group, wherein at least one of R³⁸ is not a hydrogen. In certain embodiments, each R³⁸ is independently selected from hydrogen, C₁-C₈ alkyl, C₃-C₈ alkenyl, C₃-C₈ alkynyl, C₆-C₁₀ aryl, 5-10 membered heteroaryl, 4-10 membered heterocyclyl, or C₃-C₁₀ cycloalkyl; or C₁-C₈ alkyl, substituted with halo or hydroxy; C₃-C₈ alkenyl, substituted with halo or hydroxy; C₃-C₈ alkynyl, substituted with halo or hydroxy, or —(CH₂)ₜ(C₆-C₁₀ aryl), —(CH₂)ₜ(5-10 membered heteroaryl), —(CH₂)ₜ(C₃-C₁₀ cycloalkyl), or —(CH₂)ₜ(4-10 membered heterocyclyl), wherein t is an integer between 0 and 8, each of which is substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy; or both R³⁸ groups are joined to form an alkylene group.

Exemplary “substituted amino” groups include, but are not limited to, —NR³⁹—C₁-C₈ alkyl, —NR³⁹—(CH₂)ₜ(C₆-C₁₀ aryl), —NR³⁹—(CH₂)ₜ(5-10 membered heteroaryl), —NR³⁹—(CH₂)ₜ(C₃-C₁₀ cycloalkyl), and —NR³⁹—(CH₂)ₜ(4-10 membered heterocyclyl), wherein t is an integer from 0 to 4, for instance 1 or 2, each R³⁹ independently represents H or C₁-C₈ alkyl; and any alkyl groups present, may themselves be substituted by halo, substituted or unsubstituted amino, or hydroxy; and any aryl, heteroaryl, cycloalkyl, or heterocyclyl groups present, may themselves be substituted by unsubstituted C₁-C₄ alkyl, halo, unsubstituted C₁-C₄ alkoxy, unsubstituted C₁-C₄ haloalkyl, unsubstituted C₁-C₄ hydroxyalkyl, or unsubstituted C₁-C₄ haloalkoxy or hydroxy. For the avoidance of doubt the term ‘substituted amino’ includes the groups alkylamino, substituted alkylamino, alkylarylamino, substituted alkylarylamino, arylamino, substituted arylamino, dialkylamino, and substituted dialkylamino as defined below. Substituted amino encompasses both monosubstituted amino and disubstituted amino groups.

“Carboxy” refers to the radical —C(O)OH.

“Cyano” refers to the radical —CN.

“Halo” or “halogen” refers to fluoro (F), chloro (Cl), bromo (Br), and iodo (I). In certain embodiments, the halo group is either fluoro or chloro.

“Hydroxy” refers to the radical —OH.

“Nitro” refers to the radical —NO₂.

“Cycloalkylalkyl” refers to an alkyl radical in which the alkyl group is substituted with a cycloalkyl group. Typical cycloalkylalkyl groups include, but are not limited to, cyclopropylmethyl, cyclobutylmethyl, cyclopentylmethyl, cyclohexylmethyl, cycloheptylmethyl, cyclooctylmethyl, cyclopropylethyl, cyclobutylethyl, cyclopentylethyl, cyclohexylethyl, cycloheptylethyl, and cyclooctylethyl, and the like.

“Heterocyclylalkyl” refers to an alkyl radical in which the alkyl group is substituted with a heterocyclyl group. Typical heterocyclylalkyl groups include, but are not limited to, pyrrolidinylmethyl, piperidinylmethyl, piperazinylmethyl, morpholinylmethyl, pyrrolidinylethyl, piperidinylethyl, piperazinylethyl, morpholinylethyl, and the like.

“Nitrogen-containing heterocyclyl” group means a 4- to 7-membered non-aromatic cyclic group containing at least one nitrogen atom, for example, but without limitation, morpholine, piperidine (e.g. 2-piperidinyl, 3-piperidinyl and 4-piperidinyl), pyrrolidine (e.g. 2-pyrrolidinyl and 3-pyrrolidinyl), azetidine, pyrrolidone, imidazoline, imidazolidinone, 2-pyrazoline, pyrazolidine, piperazine, and N-alkyl piperazines such as N-methyl piperazine. Particular examples include azetidine, piperidone and piperazone.

“Thioketo” refers to the group ═S.

Alkyl, alkenyl, alkynyl, carbocyclyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR)₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc}; each instance of R^{aa} is, independently, selected from C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O) SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, OC(═O)N(R^{ff})₂, —NR^{ff}C(═O)R^{ee}, —NR^{ff}CO₂R^{ee}, —NR^{ee}(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O) SR^{ee}, —C(═S)SR^{ee}, —SC(═S) SR^{ee}, —P(═O)₂R^{ee}, —P(═O)(R^{ee})₂, —OP(═O)(R^{ee})₂, —OP(═O)(OR^{ee})₂, C₁₋₆alkyl, C₁₋₆ perhaloalkyl, C₂₋₆alkenyl, C₂₋₆alkynyl, C₃₋₁₀carbocyclyl, 3-10 membered heterocyclyl, C₆₋₁₀ aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;
each instance of R^{ee} is, independently, selected from C₁₋₆alkyl, C₁₋₆ perhaloalkyl, C₂₋₆alkenyl, C₂₋₆alkynyl, C₃₋₁₀carbocyclyl, C₆₋₁₀ aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₆alkyl, C₁₋₆ perhaloalkyl, C₂₋₆alkenyl, C₂₋₆alkynyl, C₃₋₁₀carbocyclyl, 3-10 membered heterocyclyl, C₆₋₁₀ aryl and 5-10 membered heteroaryl, or two R^{ff} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆alkyl, —ON(C₁₋₆alkyl)₂, —N(C₁₋₆alkyl)₂, —N(C₁₋₆alkyl)₃⁺X⁻, —NH(C₁₋₆ alkyl)₂⁺X⁻, —NH₂ (C₁₋₆alkyl)⁺X⁻, —NH₃⁺X⁻, —N(OC₁₋₆alkyl) (C₁₋₆alkyl), —N(OH) (C₁₋₆alkyl), —NH(OH), —SH, —SC₁₋₆alkyl, —SS(C₁₋₆alkyl), —C(═O) (C₁₋₆alkyl), —CO₂H, —CO₂(C₁₋₆ alkyl), —OC(═O)(C₁₋₆alkyl), —OCO₂(C₁₋₆alkyl), —C(═O)NH₂, —C(═O)N(C₁₋₆alkyl)₂, —OC(═O)NH(C₁₋₆alkyl), —NHC(═O) (C₁₋₆alkyl), —N(C₁₋₆alkyl) C(═O) (C₁₋₆alkyl), —NHCO₂(C₁₋₆alkyl), —NHC(═O)N(C₁₋₆alkyl)₂, —NHC(═O)NH(C₁₋₆alkyl), —NHC(═O)NH₂, —C(═NH)O(C₁₋₆alkyl), —OC(═NH)(C₁₋₆alkyl), —OC(═NH)OC₁₋₆alkyl, —C(═NH)N(C₁₋₆ alkyl)₂, —C(═NH)NH(C₁₋₆alkyl), —C(═NH)NH₂, —OC(═NH)N(C₁₋₆alkyl)₂, —OC(NH)NH(C₁₋₆alkyl), —OC(NH)NH₂, —NHC(NH)N(C₁₋₆alkyl)₂, —NHC(═NH)NH₂, —NHSO₂(C₁₋₆alkyl), —SO₂N(C₁₋₆alkyl)₂, —SO₂NH(C₁₋₆alkyl), —SO₂NH₂, —SO₂C₁₋₆alkyl, —SO₂OC₁₋₆alkyl, —OSO₂C₁₋₆alkyl, —SOC₁₋₆alkyl, —Si(C₁₋₆alkyl)₃, —OSi(C₁₋₆alkyl)₃-C(═S)N(C₁₋₆alkyl)₂, C(═S)NH(C₁₋₆alkyl), C(═S)NH₂, —C(═O)S(C₁₋₆alkyl), —C(═S)SC₁₋₆ alkyl, —SC(═S)SC₁₋₆alkyl, —P(═O)₂(C₁₋₆alkyl), —P(═O)(C₁₋₆alkyl)₂, —OP(═O)(C₁₋₆alkyl)₂, —OP(═O)(OC₁₋₆alkyl)₂, C₁₋₆alkyl, C₁₋₆ perhaloalkyl, C₂₋₆alkenyl, C₂₋₆alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

A “counterion” or “anionic counterion” is a negatively charged group associated with a cationic quaternary amino group in order to maintain electronic neutrality. Exemplary counterions include halide ions (e.g., F⁻, Cl⁻, Br⁻, I⁻), NO₃⁻, ClO₄⁻, OH⁻, H₂PO₄⁻, HSO₄⁻, SO₄⁻² sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{aa}, —C(═S) SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

### Other Definitions

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound disclosed herein that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like. The term “pharmaceutically acceptable cation” refers to an acceptable cationic counterion of an acidic functional group. Such cations are exemplified by sodium, potassium, calcium, magnesium, ammonium, tetraalkylammonium cations, and the like. See, e.g., Berge, et al., J. Pharm. Sci. (1977) 66 (1): 1-79.

“Pharmaceutically acceptable carrier” refers to compositions, carriers, diluents, and reagents which are pharmaceutically acceptable materials that are capable of administration to or upon a subject. A pharmaceutically acceptable carrier can be involved with carrying or transporting the subject agents from one organ, or portion of the body, to another organ, or portion of the body. The carrier can be in the form of a solid, semi-solid or liquid diluent, cream or a capsule. The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof.

A “subject” to which administration is contemplated includes, but is not limited to, human subject (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” and “subject” are used interchangeably herein.

Disease, disorder, and condition are used interchangeably herein.

As used herein, the term “treat,” “treating” or “treatment” includes reversing, reducing, or arresting the symptoms, clinical signs, and underlying pathology of a condition in manner to improve or stabilize a subject's condition. As used herein, and as well understood in the art, “treatment” is an approach for obtaining beneficial or desired results, including clinical results. Beneficial or desired clinical results can include, but are not limited to, alleviation, amelioration, reduction of the severity, or slowing the progression, of one or more symptoms or conditions associated with a condition, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Exemplary beneficial clinical results are described herein.

As used herein, and unless otherwise specified, the term “prophylactic,” “prevention” and variations thereof, contemplates an action that occurs before a subject begins to suffer from the specified disease, disorder, or condition.

In general, the “effective amount” of a compound refers to an amount sufficient to elicit the desired biological response. As will be appreciated by those of ordinary skill in this art, the effective amount of a compound of the disclosure may vary depending on such factors as the desired biological endpoint, the pharmacokinetics of the compound, the disease being treated, the mode of administration, and the age, weight, health, and condition of the subject. An effective amount encompasses therapeutic and prophylactic treatment.

The terms “pharmaceutically effective amount,” “therapeutically effective amount,” or “therapeutically effective dose” refer to an amount sufficient to treat a disease in a patient, e.g., effecting a beneficial and/or desirable alteration in the health of a patient suffering from a disease, treatment, healing, inhibition or amelioration of a physiological response or condition, delaying or minimizing one or more symptoms associated with the disease, disorder or condition etc. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, the nature and extent of disease, the therapeutics or combination of therapeutics selected for administration, and the mode of administration. The skilled worker can readily determine the effective amount for a given situation by routine experimentation. The terms “pharmaceutically effective amount,” “therapeutically effective amount,” or “therapeutically effective dose” also refer to the amount required to improve the clinical symptoms of a patient. A therapeutically effective amount of a compound also refers to an amount of the therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

As used herein, and unless otherwise specified, a “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent.

As used herein, and unless otherwise specified, “pharmacokinetics” can be defined as the study of bodily absorption, distribution, metabolism, and excretion of drugs. “Pharmacokinetics” can also be defined as the characteristic interactions of a drug and a body in terms of its absorption, distribution, metabolism, and excretion; or a branch of pharmacology concerned with the way drugs are taken into, move around, and are eliminated from, a body.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods. In some embodiments, the administration includes both direct administration, including self-administration, and indirect administration, including the act of prescribing a drug. For example, as used herein, a physician who instructs a patient to self-administer a drug, or to have the drug administered by another and/or who provides a patient with a prescription for a drug is administering the drug to the patient. When a method is part of a therapeutic regimen involving more than one agent or treatment modality, the disclosure contemplates that the agents may be administered at the same or differing times and via the same or differing routes of administration. Appropriate methods of administering a substance, a compound or an agent to a subject will also depend, for example, on the age of the subject, whether the subject is active or inactive at the time of administering, whether the subject is cognitively impaired at the time of administering, the extent of the impairment, and the chemical and biological properties of the compound or agent (e.g. solubility, digestibility, bioavailability, stability and toxicity).

As generally described herein, the disclosure provides compounds useful for preventing and/or treating a broad range of disorders, including, but not limited to, NMDA receptor-mediated disorders. These compounds are expected to show improved in vivo potency, pharmacokinetic (PK) properties, oral bioavailability, formulatability, stability, and/or safety as compared to other compounds.

### Compounds

In one aspect, the disclosure provides a compound of Formula (A-I):

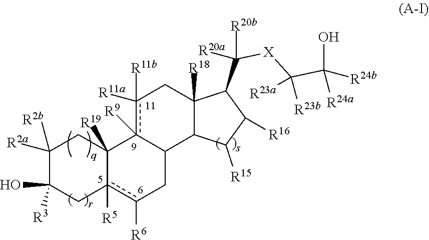

or a pharmaceutically acceptable salt thereof;
wherein:
   each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein R^{A2} is hydrogen or substituted or unsubstituted C₁₋₆alkyl;
   R³ is substituted or unsubstituted C₁₋₆alkyl, substituted or unsubstituted C₂₋₆ alkenyl, substituted or unsubstituted C₂₋₆alkynyl, or substituted or unsubstituted C₃₋₁₀ carbocyclyl;
   R⁵ is absent, hydrogen, halogen, or substituted or unsubstituted C₁₋₆alkyl;
   R⁶ is hydrogen, halogen, or substituted or unsubstituted C₁₋₆alkyl;
   R⁹ is absent or hydrogen; or
   R⁹ and R^{11a} are taken together with the carbon atoms to which they are attached to form a substituted or unsubstituted C₃₋₆carbocyclyl;
   R^{11a} is hydrogen, halogen, substituted or unsubstituted C₁₋₆alkyl, or —OR^{A11}, wherein R^{A11} is hydrogen or substituted or unsubstituted C₁₋₆alkyl;
   R^{11b} is absent or hydrogen; or
   R^{11a} and R^{11b} are joined to form an oxo (═O) group;
   R¹⁵ is hydrogen, halogen, —OH, —NH₂, substituted or unsubstituted C₁₋₆alkyl, substituted or unsubstituted C₂₋₆alkenyl, substituted or unsubstituted C₂₋₆alkynyl, substituted or unsubstituted C₁₋₆alkoxy, substituted or unsubstituted C₃₋₆carbocyclyl, or substituted or unsubstituted 3- to 10-membered heterocyclyl;
   R¹⁶ is hydrogen, halogen, —OH, —NH₂, substituted or unsubstituted C₁₋₆alkyl, substituted or unsubstituted C₂₋₆alkenyl, substituted or unsubstituted C₂₋₆alkynyl, substituted or unsubstituted C₁₋₆alkoxy, substituted or unsubstituted C₃₋₆carbocyclyl, or substituted or unsubstituted 3- to 10-membered heterocyclyl; or
   R¹⁵ and R¹⁶ are taken together with the carbon atoms to which they are attached, to form a substituted or unsubstituted C₃₋₆carbocyclyl;
   R¹⁸ is hydrogen, substituted or unsubstituted C₁₋₆alkyl, or OR^{A18}, wherein each instance of R^{A18} is independently substituted or unsubstituted C₁₋₆alkyl.
   R¹⁹ is hydrogen, substituted or unsubstituted C₁₋₆alkyl, or OR^{A19}; wherein each instance of R^{A19} is independently substituted or unsubstituted C₁₋₆alkyl;
   R^{20a} is halogen, cyano, substituted C₁₋₆alkyl, unsubstituted C₂₋₆alkyl, substituted or unsubstituted C₁₋₆alkoxy, or substituted or unsubstituted C₃₋₆carbocyclyl;
   R^{20b} is hydrogen, halogen, cyano, substituted or unsubstituted C₁₋₆alkyl, substituted or unsubstituted C₁₋₆alkoxy, or substituted or unsubstituted C₃₋₆carbocyclyl;
   or R^{20a} and R^{20b} are both simultaneously —CH₃;
   X is —(C(R^{X})₂)ₙ— or —O—, wherein R^{X} is hydrogen or fluorine, or one R^{X} group and R^{23b} are joined to form a double bond;
   n is 1, 2 or 3;
   each instance of R^{23a} and R^{23b} is independently hydrogen or fluorine;
   R^{24a} is selected from the group consisting of substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl;
   R^{24b} is hydrogen, substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, or substituted or unsubstituted 5- to 10-membered heteroaryl;
   or R^{24a} and R^{24b}, taken together with the carbon atom to which they are attached, form a substituted or unsubstituted C₃₋₆carbocyclyl or a substituted or unsubstituted 3- to 10-membered heterocyclyl;
   q is 0, 1, or 2;
   r is 1 or 2;
   s is 1, 2, or 3; and
   each [symbol] independently represents a single or double bond, provided if the bond between C5 and C6 is a double bond, R⁵ is absent and if the bond between C9 and C11 is a double bond, R⁹ and R^{11b} are absent.

In some embodiments, the compound of formula (A-I) is a compound of formula (A-II):

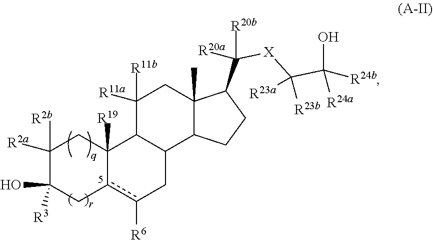

or a pharmaceutically acceptable salt thereof.

In some embodiments, the compound of formula (A-II) is a compound of formula (A-II-1) or formula (A-II-2):

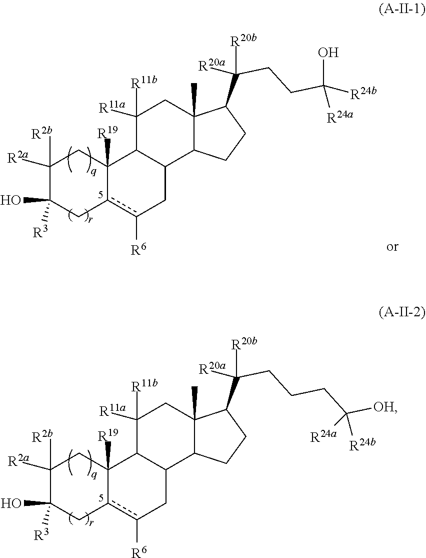

or a pharmaceutically acceptable salt thereof.

In some embodiments, the compound of formula (A-II-1) is a compound of formula (A-II-1-A) or formula (A-II-1-B):

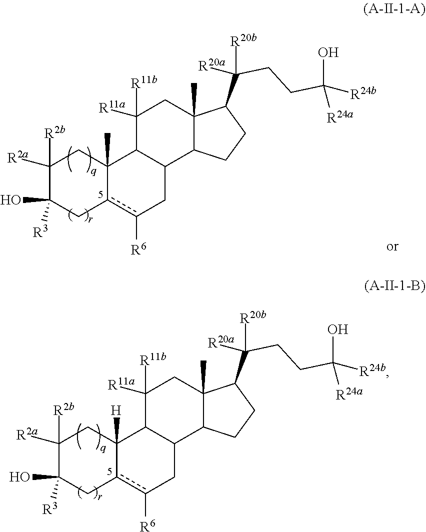

or a pharmaceutically acceptable salt thereof.

In some embodiments, the compound of formula (A-II-1-A) is a compound of formula (A-II-1-A-1) or formula (A-II-1-A-2):

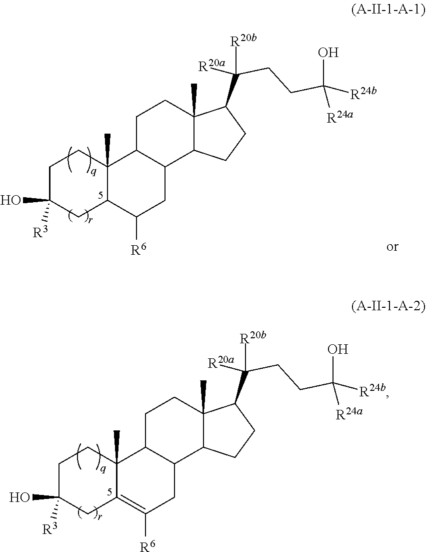

or a pharmaceutically acceptable salt thereof.

In some embodiments, the compound of formula (A-II-1-A-1) is a compound of formula (A-II-1-A-1-a):

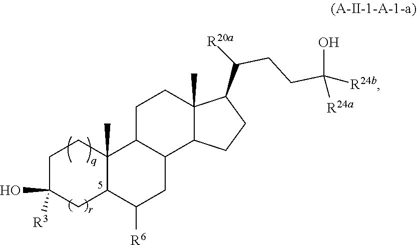

or a pharmaceutically acceptable salt thereof.

In some embodiments, the compound of formula (A-II-1-A-1-a) is a compound of formula (A-II-1-A-1-a-i) or (A-II-1-A-1-a-ii):

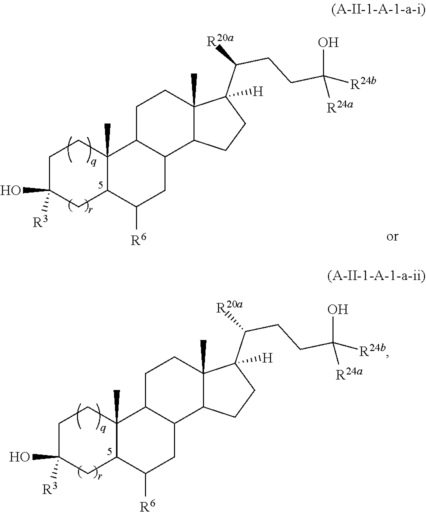

or a pharmaceutically acceptable salt thereof.

In some embodiments, the compound of formula (A-II-1-A-1-a-i) is a compound of formula (A-II-1-A-1-a-iii) or (A-II-1-A-1-a-iv):

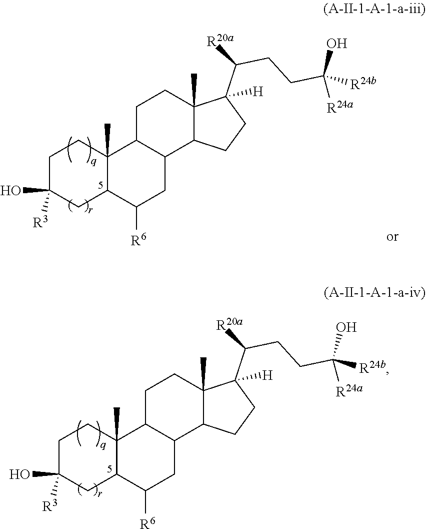

or a pharmaceutically acceptable salt thereof.

In some embodiments, the compound of formula (A-II-1-A-1-a-ii) is a compound of formula (A-II-1-A-1-a-v) or (A-II-1-A-1-a-vi):

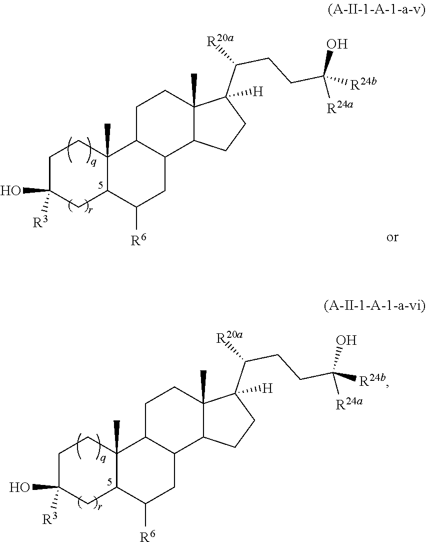

or a pharmaceutically acceptable salt thereof.

In some embodiments, the compound of formula (A-II-1-A-2) is a compound of formula (A-II-1-A-2-a):

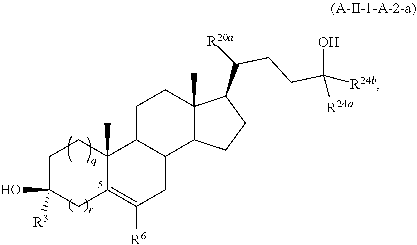

or a pharmaceutically acceptable salt thereof.

In some embodiments, the compound of formula (A-II-1-A-2-a) is a compound of formula (A-II-1-A-2-a-i) or (A-II-1-A-2-a-ii):

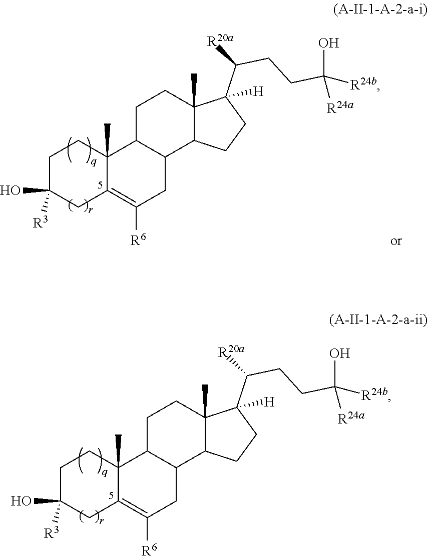

or a pharmaceutically acceptable salt thereof.

In some embodiments, the compound of formula (A-II-1-A-2-a-i) is a compound of formula (A-II-1-A-2-a-iii) or (A-II-1-A-2-a-iv):

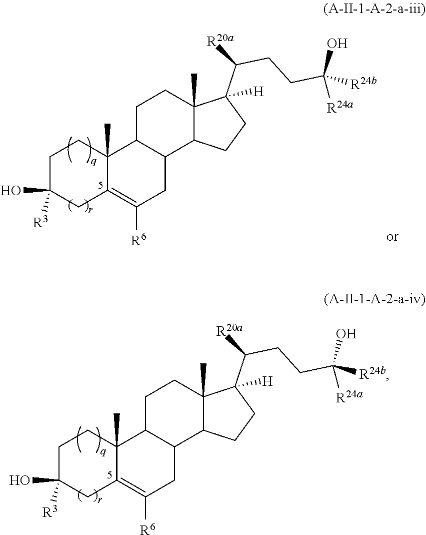

or a pharmaceutically acceptable salt thereof.

In some embodiments, the compound of formula (A-II-1-A-2-a-ii) is a compound of formula (A-II-1-A-2-a-v) or (A-II-1-A-2-a-vi):

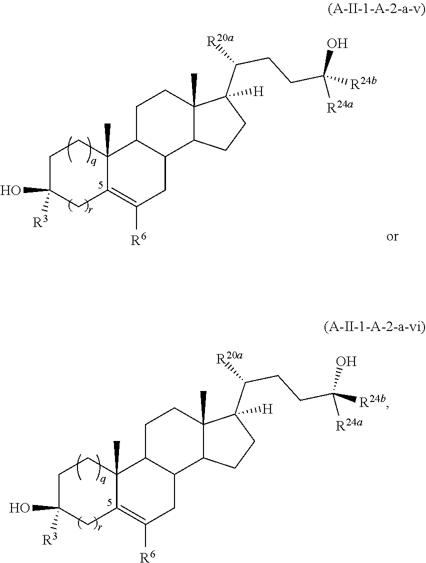

or a pharmaceutically acceptable salt thereof.

In some embodiments, the compound of formula (A-II-1-B) is a compound of formula (A-II-1-B-1) or formula (A-II-1-B-2):

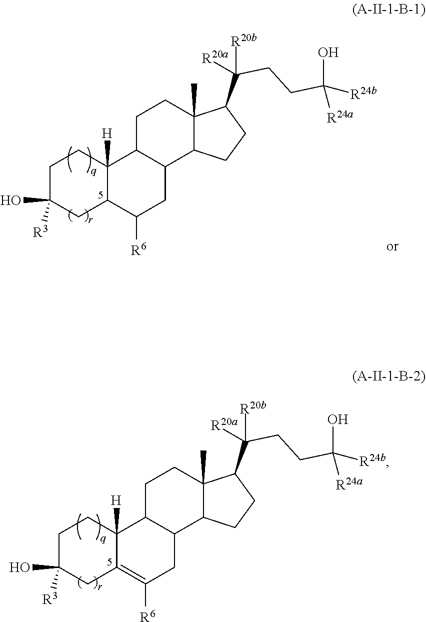

or a pharmaceutically acceptable salt thereof.

In some embodiments, the compound of formula (A-II-1-B-1) is a compound of formula (A-II-1-B-1-a):

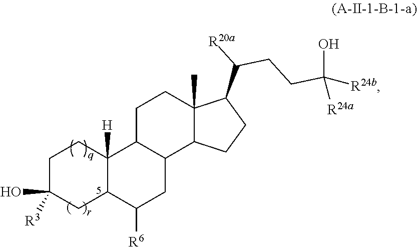

or a pharmaceutically acceptable salt thereof.

In some embodiments, the compound of formula (A-II-1-B-1-a) is a compound of formula (A-II-1-B-1-a-i) or formula (A-II-1-B-1-a-ii):

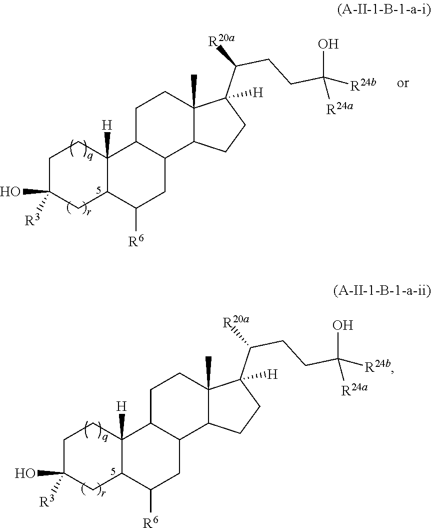

or a pharmaceutically acceptable salt thereof.

In some embodiments, the compound of formula (A-II-1-B-1-a-i) is a compound of formula (A-II-1-B-1-a-iii) or formula (A-II-1-B-1-a-iv):

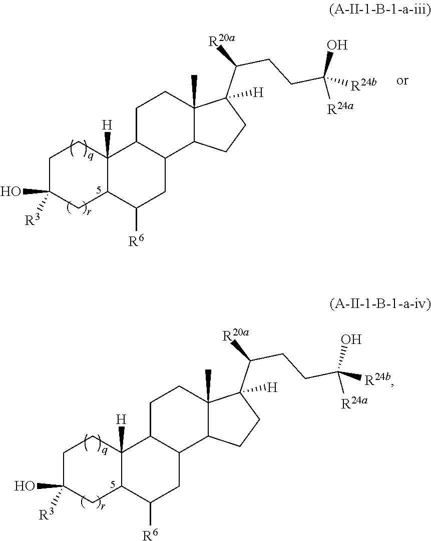

or a pharmaceutically acceptable salt thereof.

In some embodiments, the compound of formula (A-II-1-B-1-a-ii) is a compound of formula (A-II-1-B-1-a-v) or formula (A-II-1-B-1-a-vi):

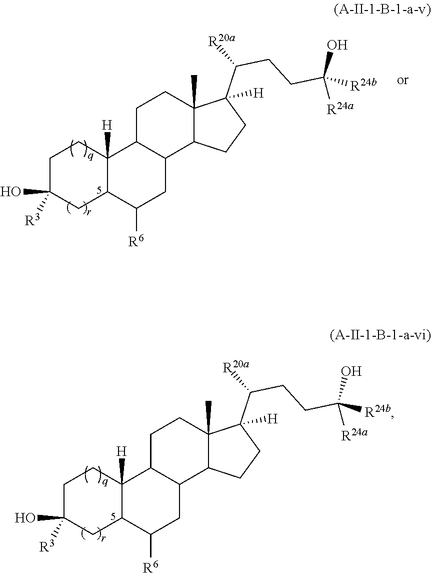

or a pharmaceutically acceptable salt thereof.

In some embodiments, the compound of formula (A-II-1-B-2) is a compound of formula (A-II-1-B-2-a):

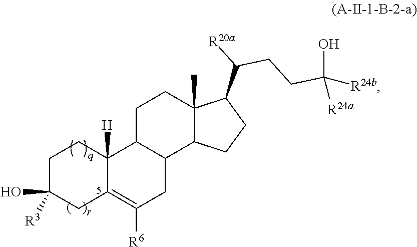

or a pharmaceutically acceptable salt thereof.

In some embodiments, the compound of formula (A-II-1-B-2-a) is a compound of formula (A-II-1-B-2-a-i) or formula (A-II-1-B-2-a-ii):

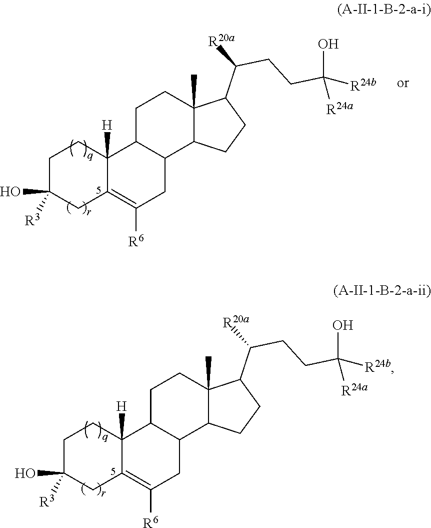

or a pharmaceutically acceptable salt thereof.

In some embodiments, the compound of formula (A-II-1-B-2-a-i) is a compound of formula (A-II-1-B-2-a-iii) or formula (A-II-1-B-2-a-iv):

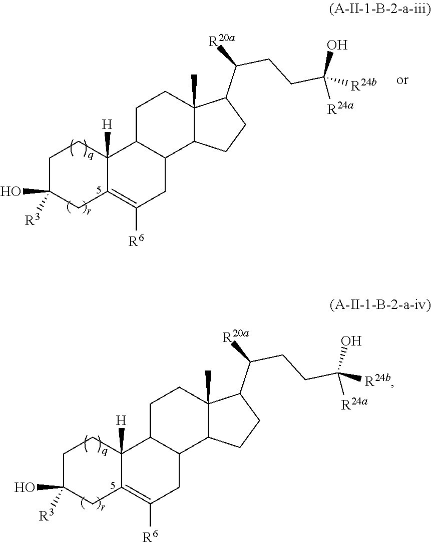

or a pharmaceutically acceptable salt thereof.

In some embodiments, the compound of formula (A-II-1-B-2-a-ii) is a compound of formula (A-II-1-B-2-a-v) or formula (A-II-1-B-2-a-vi):

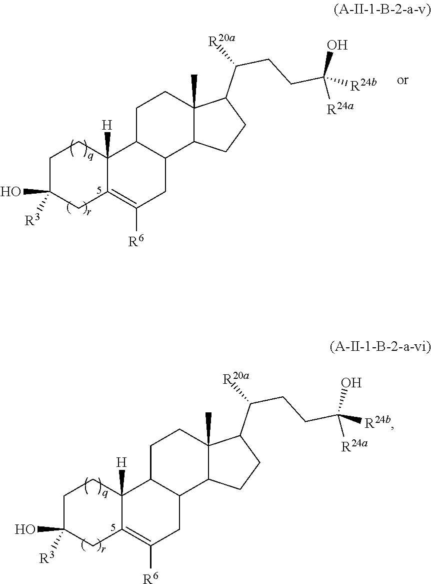

or a pharmaceutically acceptable salt thereof.

In some embodiments, the compound of formula (A-II-2) is a compound of formula (A-II-2-A) or formula (A-II-2-B):

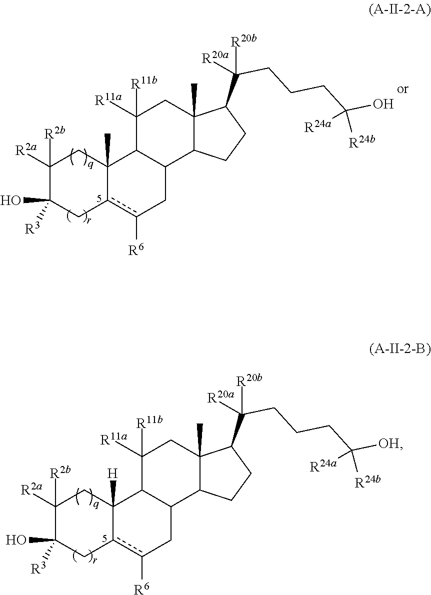

or a pharmaceutically acceptable salt thereof.

In some embodiments, the compound of formula (A-II-2-A) is a compound of formula (A-II-2-A-1) or formula (A-II-2-A-2):

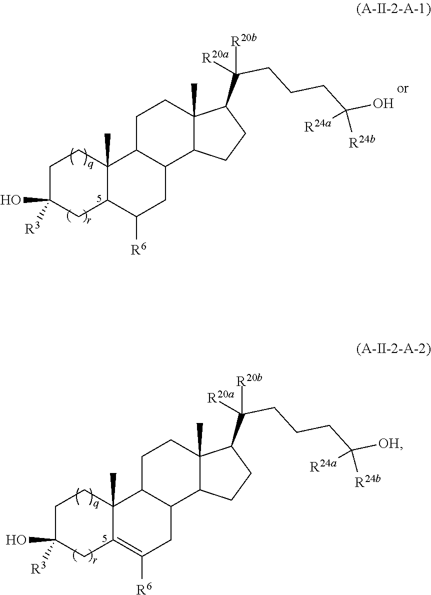

or a pharmaceutically acceptable salt thereof.

In some embodiments, the compound of formula (A-II-2-A-1) is a compound of formula (A-II-2-A-1-a):

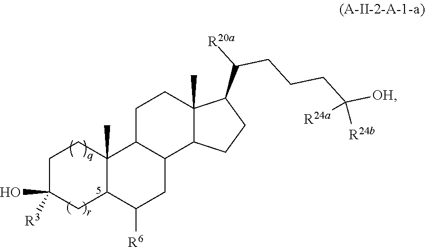

or a pharmaceutically acceptable salt thereof.

In some embodiments, the compound of formula (A-II-2-A-1-a) is a compound of formula (A-II-2-A-1-a-i) or formula (A-II-2-A-1-a-ii):

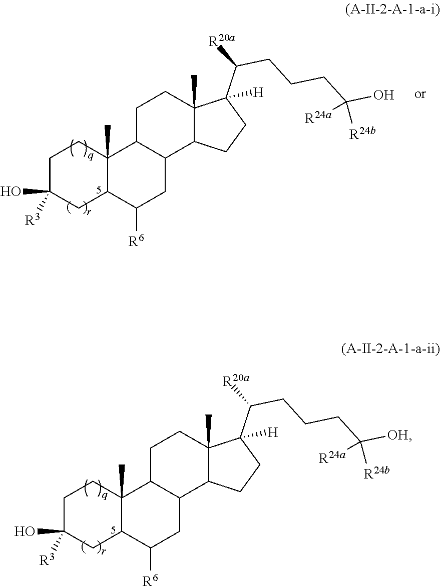

or a pharmaceutically acceptable salt thereof.

In some embodiments, the compound of formula (A-II-2-A-1-a-i) is a compound of formula (A-II-2-A-1-a-iii) or formula (A-II-2-A-1-a-iv):

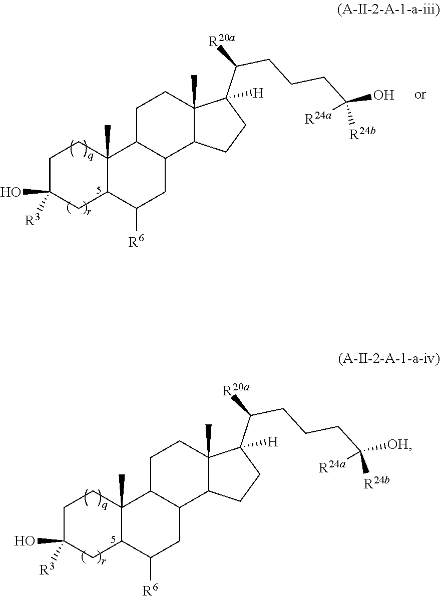

or a pharmaceutically acceptable salt thereof.

In some embodiments, the compound of formula (A-II-2-A-1-a-ii) is a compound of formula (A-II-2-A-1-a-v) or formula (A-II-2-A-1-a-vi):

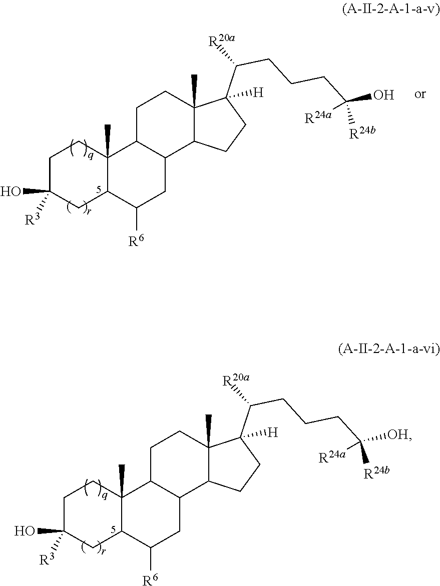

or a pharmaceutically acceptable salt thereof.

In some embodiments, the compound of formula (A-II-2-A-2) is a compound of formula (A-II-2-A-2-a):

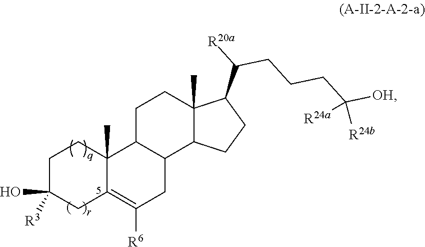

or a pharmaceutically acceptable salt thereof.

In some embodiments, the compound of formula (A-II-2-A-2-a) is a compound of formula (A-II-2-A-2-a-i) or formula (A-II-2-A-2-a-ii):

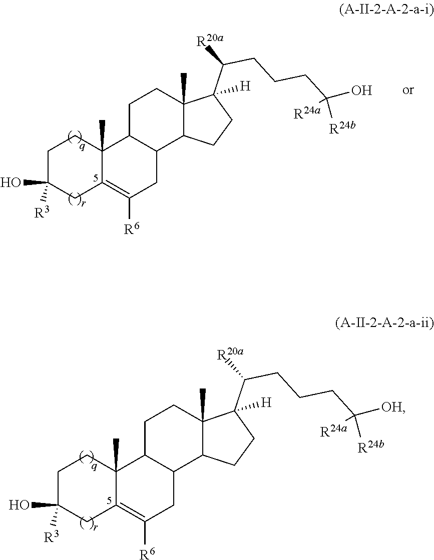

or a pharmaceutically acceptable salt thereof.

In some embodiments, the compound of formula (A-II-2-A-2-a-i) is a compound of formula (A-II-2-A-2-a-iii) or formula (A-II-2-A-2-a-iv):

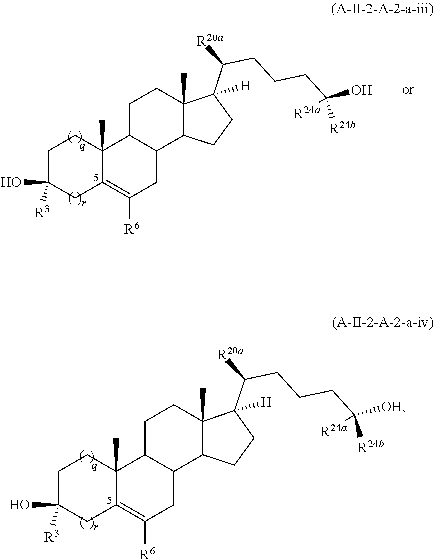

or a pharmaceutically acceptable salt thereof.

In some embodiments, the compound of formula (A-II-2-A-2-a-ii) is a compound of formula (A-II-2-A-2-a-v) or formula (A-II-2-A-2-a-vi):

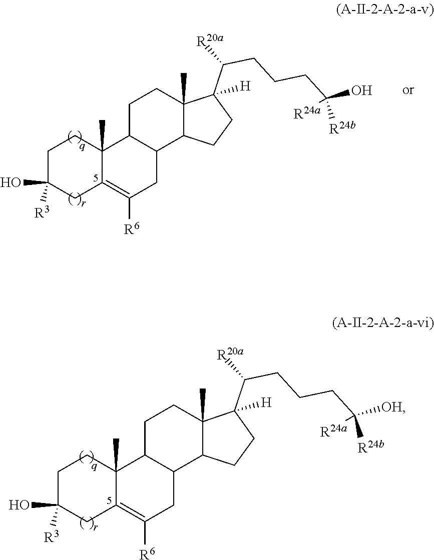

or a pharmaceutically acceptable salt thereof.

In some embodiments, the compound of formula (A-II-2-B) is a compound of formula (A-II-2-B-1) or formula (A-II-2-B-2):

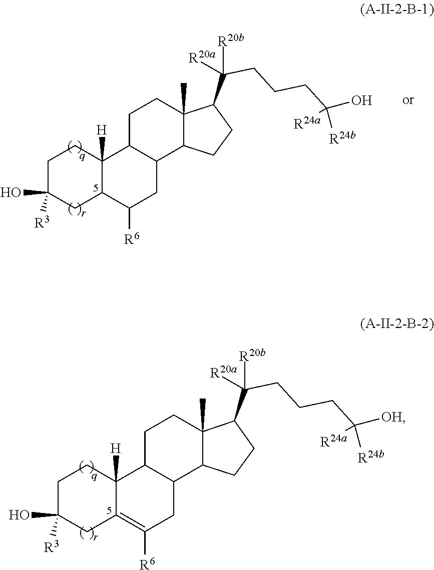

or a pharmaceutically acceptable salt thereof.

In some embodiments, the compound of formula (A-II-2-B-1) is a compound of formula (A-II-2-B-1-a):

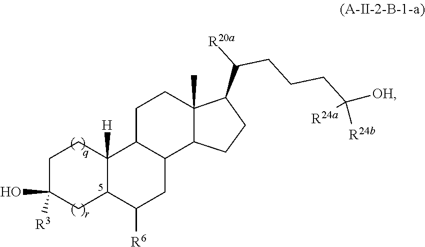

or a pharmaceutically acceptable salt thereof.

In some embodiments, the compound of formula (A-II-2-B-1-a) is a compound of formula (A-II-2-B-1-a-i) or formula (A-II-2-B-1-a-ii):

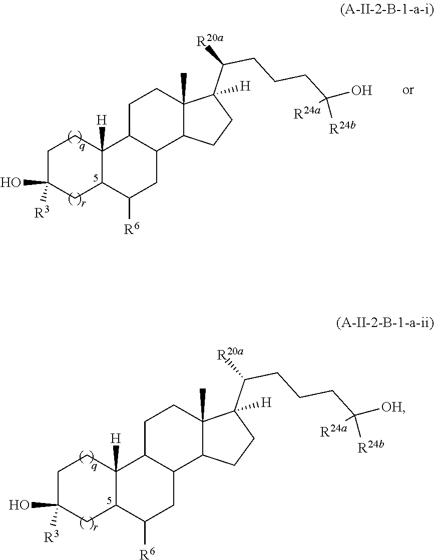

or a pharmaceutically acceptable salt thereof.

In some embodiments, the compound of formula (A-II-2-B-1-a-i) is a compound of formula (A-II-2-B-1-a-iii) or formula (A-II-2-B-1-a-iv):

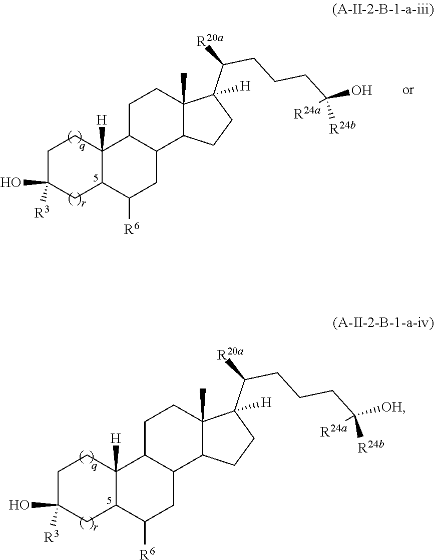

or a pharmaceutically acceptable salt thereof.

In some embodiments, the compound of formula (A-II-2-B-1-a-ii) is a compound of formula (A-II-2-B-1-a-v) or formula (A-II-2-B-1-a-vi):

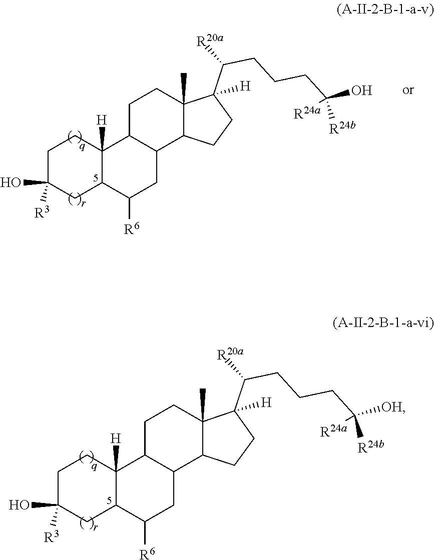

or a pharmaceutically acceptable salt thereof.

In some embodiments, the compound of formula (A-II-2-B-2) is a compound of formula (A-II-2-B-2-a):

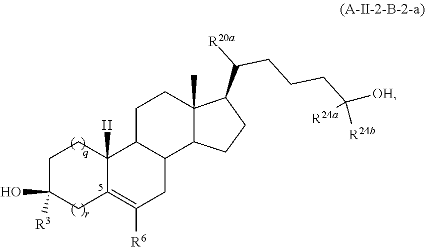

or a pharmaceutically acceptable salt thereof.

In some embodiments, the compound of formula (A-II-2-B-2-a) is a compound of formula (A-II-2-B-2-a-i) or formula (A-II-2-B-2-a-ii):

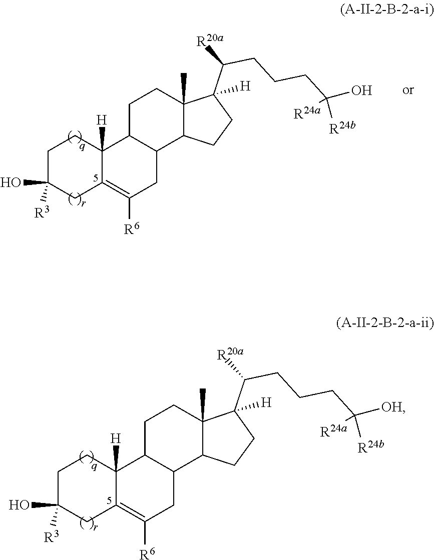

or a pharmaceutically acceptable salt thereof.

In some embodiments, the compound of formula (A-II-2-B-2-a-i) is a compound of formula (A-II-2-B-2-a-iii) or formula (A-II-2-B-2-a-iv):

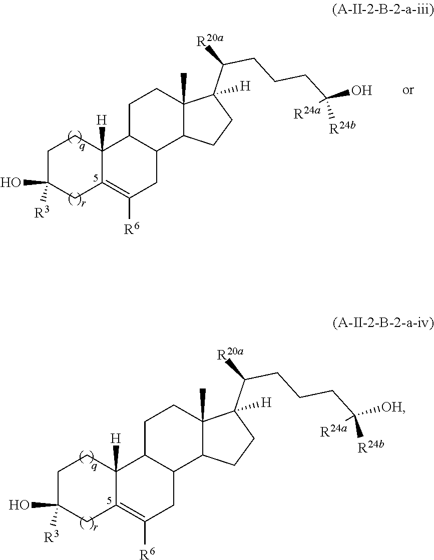

or a pharmaceutically acceptable salt thereof.

In some embodiments, the compound of formula (A-II-2-B-2-a-ii) is a compound of formula (A-II-2-B-2-a-v) or formula (A-II-2-B-2-a-vi):

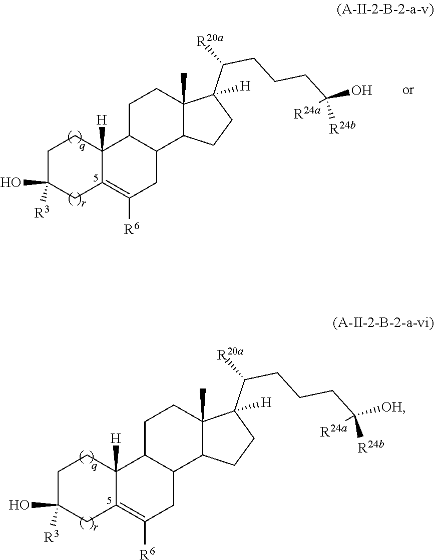

or a pharmaceutically acceptable salt thereof.

In some embodiments, the compound of formula (A-I) is a compound of formula (A-III):

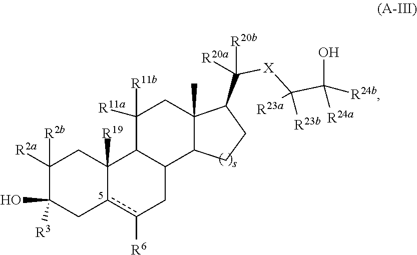

or a pharmaceutically acceptable salt thereof.

In some embodiments, the compound of formula (A-III) is a compound of formula (A-III-1) or formula (A-III-2):

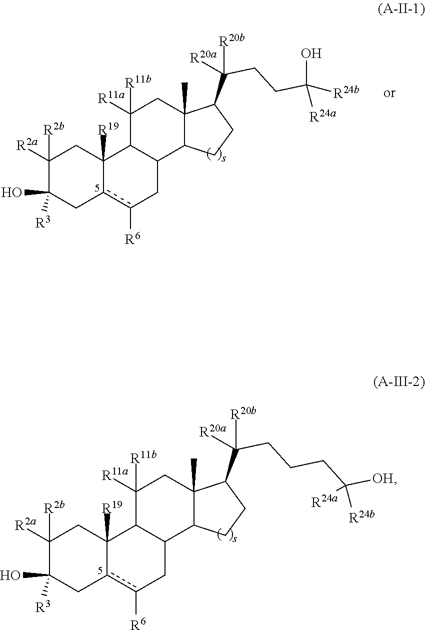

or a pharmaceutically acceptable salt thereof.

In some embodiments, the compound of formula (A-III-1) is a compound of formula (A-III-1-A) or formula (A-III-1-B):

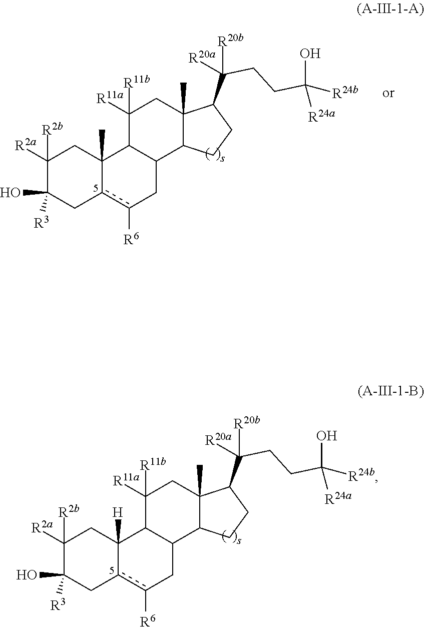

or a pharmaceutically acceptable salt thereof.

In some embodiments, the compound of formula (A-III-1-A) is a compound of formula (A-III-1-A-1) or formula (A-III-1-A-2):

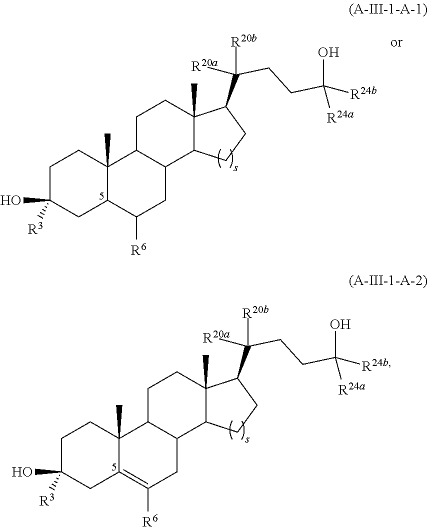

or a pharmaceutically acceptable salt thereof.

In some embodiments, the compound of formula (A-III-1-A-1) is a compound of formula (A-III-1-A-1-a):

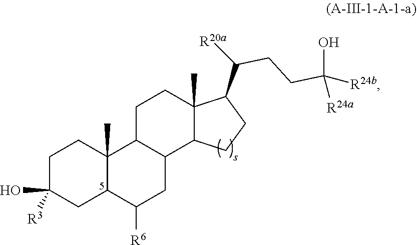

or a pharmaceutically acceptable salt thereof.

In some embodiments, the compound of formula (A-III-1-A-1-a) is a compound of formula (A-III-1-A-1-a-i) or (A-III-1-A-1-a-ii):

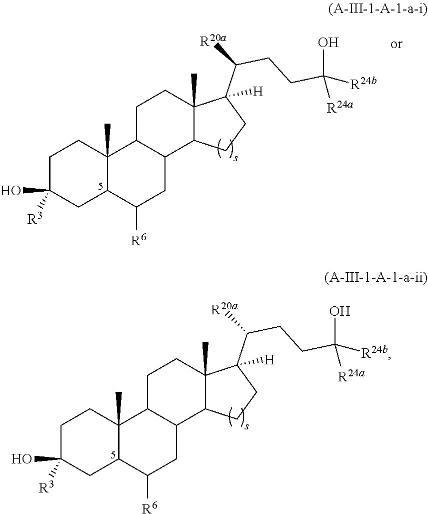

or a pharmaceutically acceptable salt thereof.

In some embodiments, the compound of formula (A-III-1-A-1-a-i) is a compound of formula (A-III-1-A-1-a-iii) or (A-III-1-A-1-a-iv):

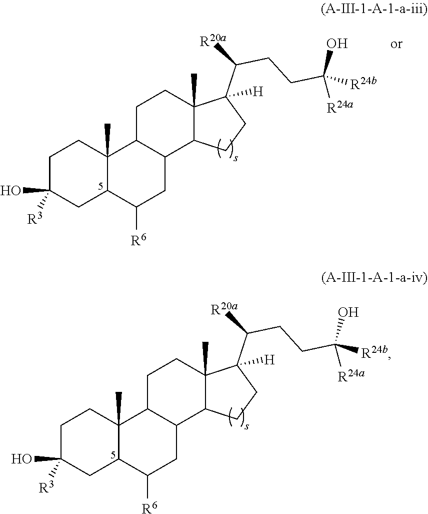

or a pharmaceutically acceptable salt thereof.

In some embodiments, the compound of formula (A-III-1-A-1-a-ii) is a compound of formula (A-III-1-A-1-a-v) or (A-III-1-A-1-a-vi):

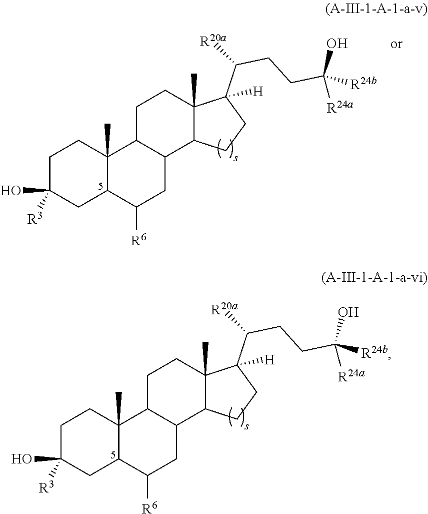

or a pharmaceutically acceptable salt thereof.

In some embodiments, the compound of formula (A-III-1-A-2) is a compound of formula (A-III-1-A-2-a):

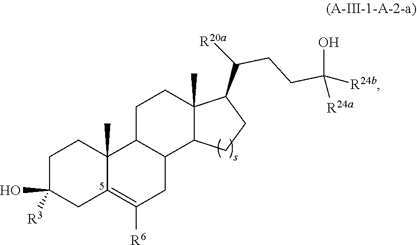

or a pharmaceutically acceptable salt thereof.

In some embodiments, the compound of formula (A-III-1-A-2-a) is a compound of formula (A-III-1-A-2-a-i) or (A-III-1-A-2-a-ii):

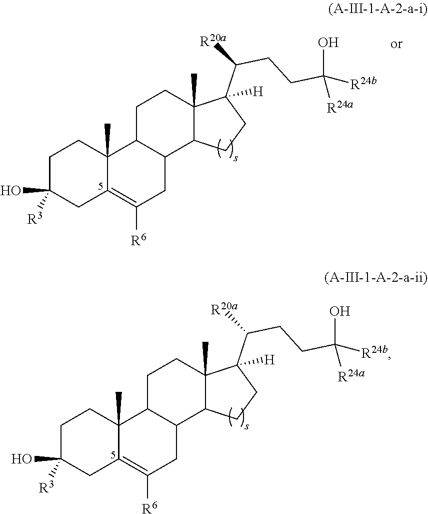

or a pharmaceutically acceptable salt thereof.

In some embodiments, the compound of formula (A-III-1-A-2-a-i) is a compound of formula (A-III-1-A-2-a-iii) or (A-III-1-A-2-a-iv):

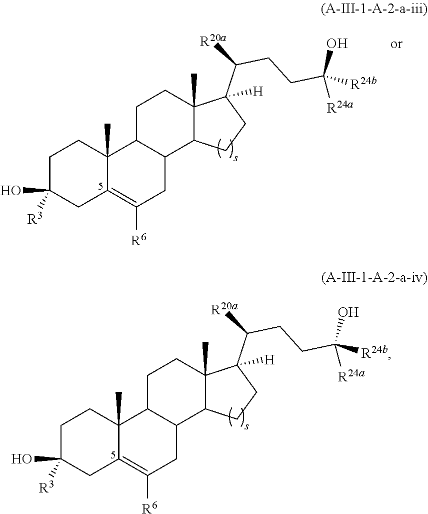

or a pharmaceutically acceptable salt thereof.

In some embodiments, the compound of formula (A-III-1-A-2-a-ii) is a compound of formula (A-III-1-A-2-a-v) or (A-III-1-A-2-a-vi):

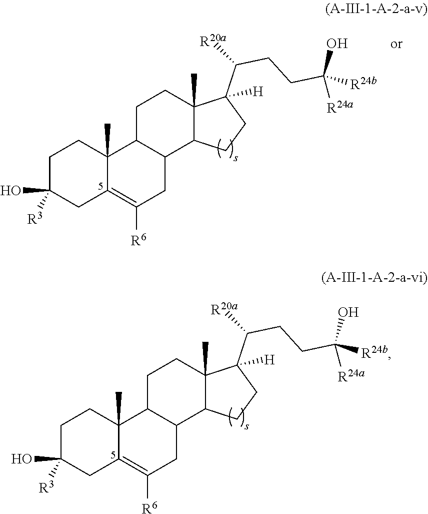

or a pharmaceutically acceptable salt thereof.

In some embodiments, the compound of formula (A-III-1-B) is a compound of formula (A-III-1-B-1) or formula (A-III-1-B-2):

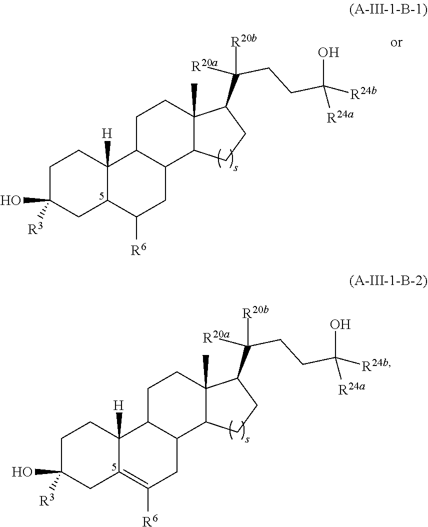

or a pharmaceutically acceptable salt thereof.

In some embodiments, the compound of formula (A-III-1-B-1) is a compound of formula (A-III-1-B-1-a):

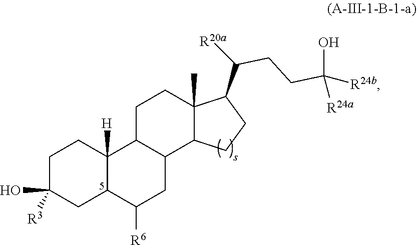

or a pharmaceutically acceptable salt thereof.

In some embodiments, the compound of formula (A-III-1-B-1-a) is a compound of formula (A-III-1-B-1-a-i) or formula (A-III-1-B-1-a-ii):

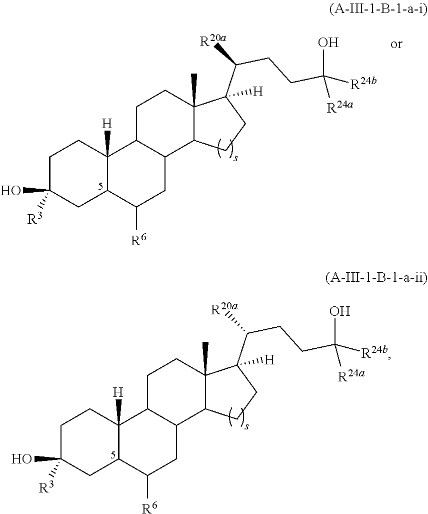

or a pharmaceutically acceptable salt thereof.

In some embodiments, the compound of formula (A-III-1-B-1-a-i) is a compound of formula (A-III-1-B-1-a-iii) or formula (A-III-1-B-1-a-iv):

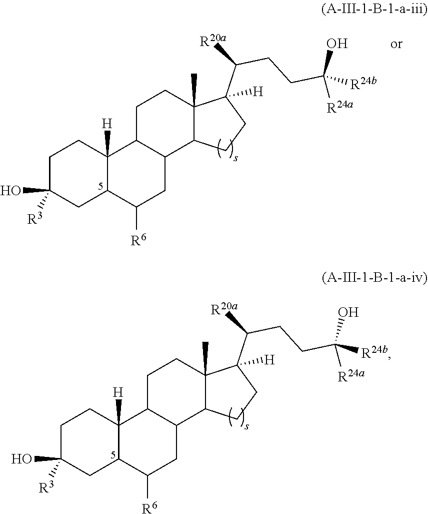

or a pharmaceutically acceptable salt thereof.

In some embodiments, the compound of formula (A-III-1-B-1-a-ii) is a compound of formula (A-III-1-B-1-a-v) or formula (A-III-1-B-1-a-vi):

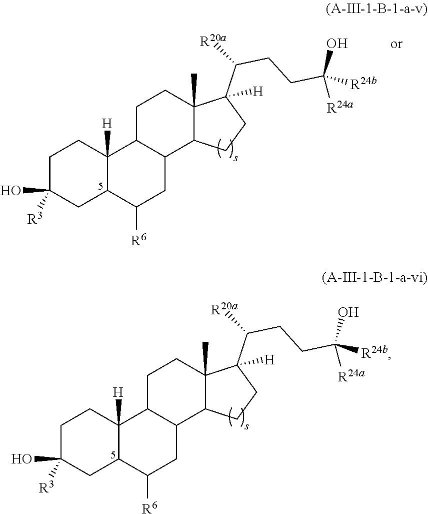

or a pharmaceutically acceptable salt thereof.

In some embodiments, the compound of formula (A-III-1-B-2) is a compound of formula (A-III-1-B-2-a):

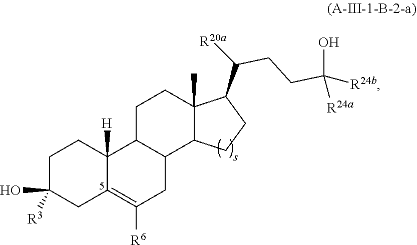

or a pharmaceutically acceptable salt thereof.

In some embodiments, the compound of formula (A-III-1-B-2-a) is a compound of formula (A-III-1-B-2-a-i) or formula (A-III-1-B-2-a-ii):

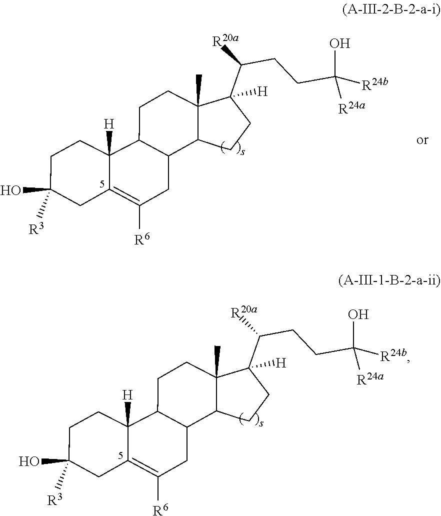

or a pharmaceutically acceptable salt thereof.

In some embodiments, the compound of formula (A-III-1-B-2-a-i) is a compound of formula (A-III-1-B-2-a-iii) or formula (A-III-1-B-2-a-iv):

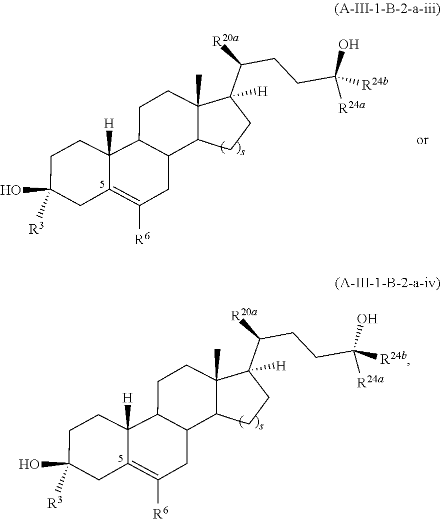

or a pharmaceutically acceptable salt thereof.

In some embodiments, the compound of formula (A-III-1-B-2-a-ii) is a compound of formula (A-III-1-B-2-a-v) or formula (A-III-1-B-2-a-vi):

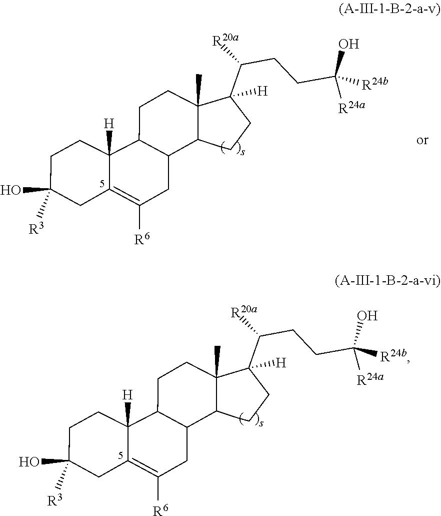

or a pharmaceutically acceptable salt thereof.

In some embodiments, the compound of formula (A-III-2) is a compound of formula (A-III-2-A) or formula (A-III-2-B):

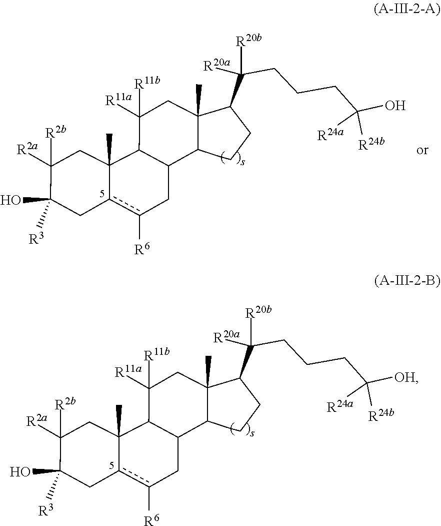

or a pharmaceutically acceptable salt thereof.

In some embodiments, the compound of formula (A-III-2-A) is a compound of formula (A-III-2-A-1) or formula (A-III-2-A-2):

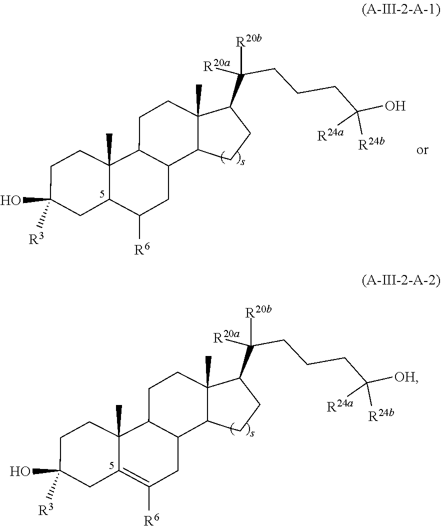

or a pharmaceutically acceptable salt thereof.

In some embodiments, the compound of formula (A-III-2-A-1) is a compound of formula (A-III-2-A-1-a):

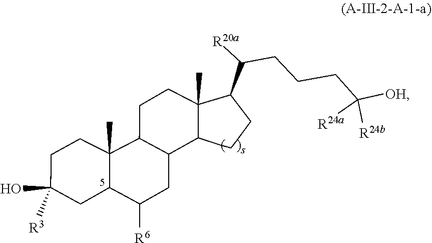

or a pharmaceutically acceptable salt thereof.

In some embodiments, the compound of formula (A-III-2-A-1-a) is a compound of formula (A-III-2-A-1-a-i) or formula (A-III-2-A-1-a-ii):

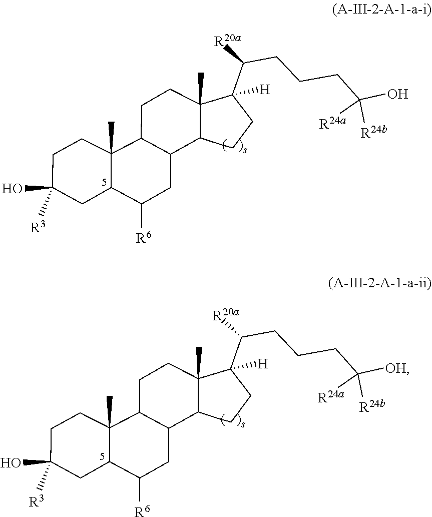

or a pharmaceutically acceptable salt thereof.

In some embodiments, the compound of formula (A-III-2-A-1-a-i) is a compound of formula (A-III-2-A-1-a-iii) or formula (A-III-2-A-1-a-iv):

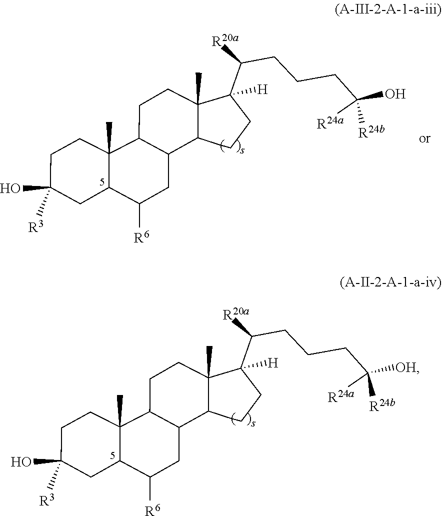

or a pharmaceutically acceptable salt thereof.

In some embodiments, the compound of formula (A-III-2-A-1-a-ii) is a compound of formula (A-III-2-A-1-a-v) or formula (A-III-2-A-1-a-vi):

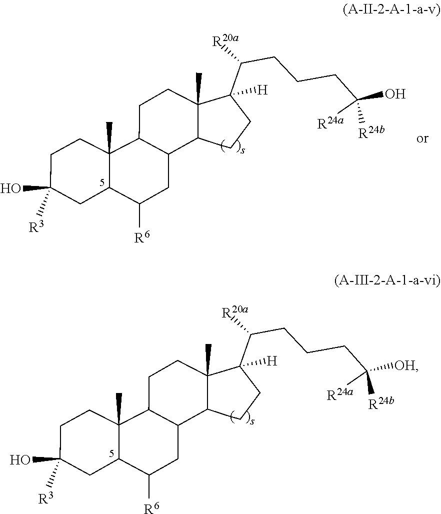

or a pharmaceutically acceptable salt thereof.

In some embodiments, the compound of formula (A-III-2-A-2) is a compound of formula (A-III-2-A-2-a):

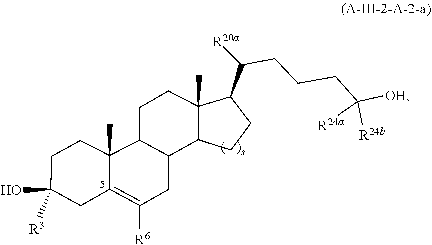

or a pharmaceutically acceptable salt thereof.

In some embodiments, the compound of formula (A-III-2-A-2-a) is a compound of formula (A-III-2-A-2-a-i) or formula (A-III-2-A-2-a-ii):

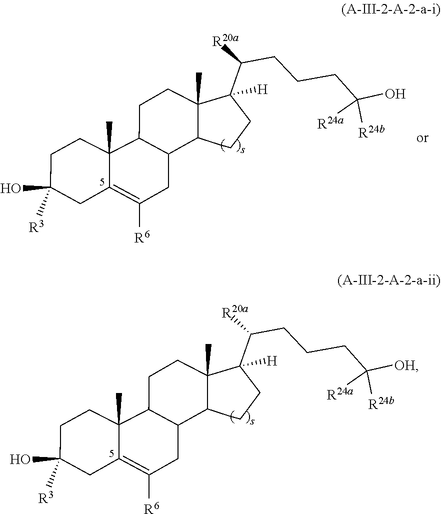

or a pharmaceutically acceptable salt thereof.

In some embodiments, the compound of formula (A-III-2-A-2-a-i) is a compound of formula (A-III-2-A-2-a-iii) or formula (A-III-2-A-2-a-iv):

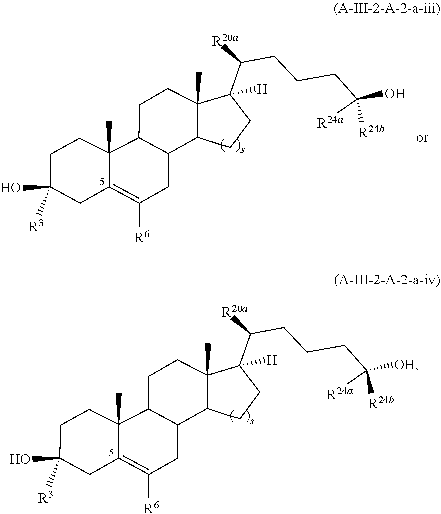

or a pharmaceutically acceptable salt thereof.

In some embodiments, the compound of formula (A-III-2-A-2-a-ii) is a compound of formula (A-III-2-A-2-a-v) or formula (A-III-2-A-2-a-vi):

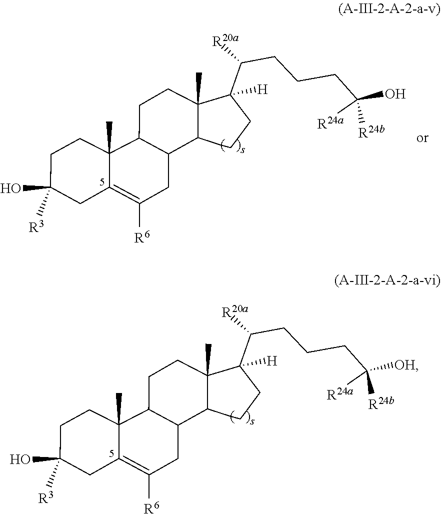

or a pharmaceutically acceptable salt thereof.

In some embodiments, the compound of formula (A-III-2-B) is a compound of formula (A-III-2-B-1) or formula (A-III-2-B-2):

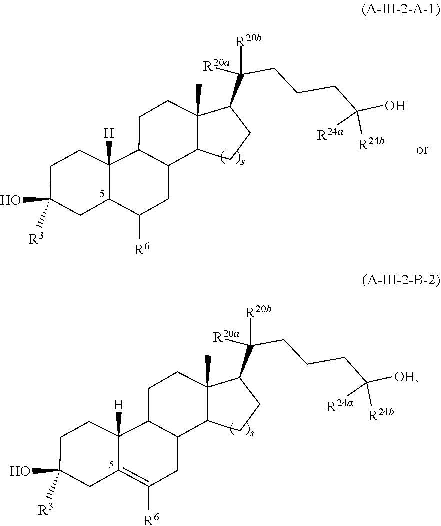

or a pharmaceutically acceptable salt thereof.

In some embodiments, the compound of formula (A-III-2-B-1) is a compound of formula (A-III-2-B-1-a):

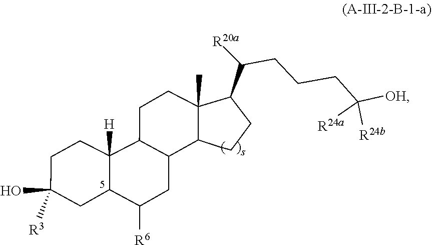

or a pharmaceutically acceptable salt thereof.

In some embodiments, the compound of formula (A-III-2-B-1-a) is a compound of formula (A-III-2-B-1-a-i) or formula (A-III-2-B-1-a-ii):

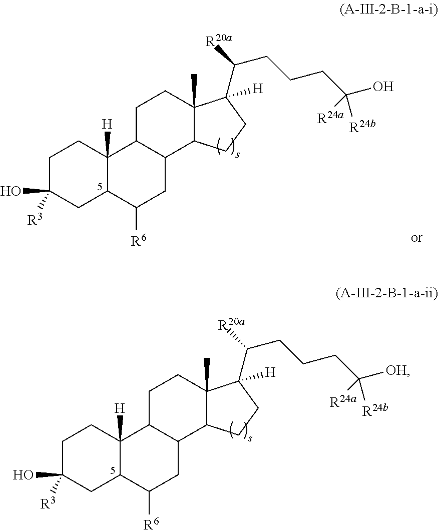

or a pharmaceutically acceptable salt thereof.

In some embodiments, the compound of formula (A-III-2-B-1-a-i) is a compound of formula (A-III-2-B-1-a-iii) or formula (A-III-2-B-1-a-iv):

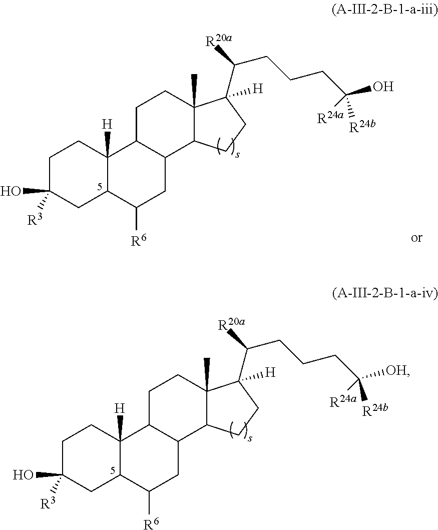

or a pharmaceutically acceptable salt thereof.

In some embodiments, the compound of formula (A-III-2-B-1-a-ii) is a compound of formula (A-III-2-B-1-a-v) or formula (A-III-2-B-1-a-vi):

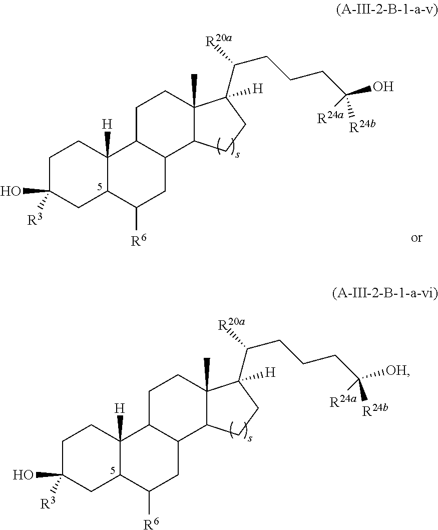

or a pharmaceutically acceptable salt thereof.

In some embodiments, the compound of formula (A-III-2-B-2) is a compound of formula (A-III-2-B-2-a):

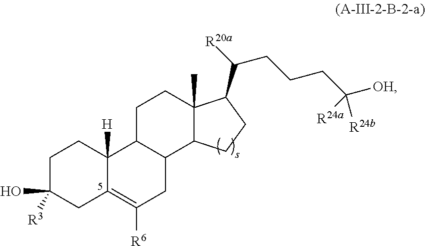

or a pharmaceutically acceptable salt thereof.

In some embodiments, the compound of formula (A-III-2-B-2-a) is a compound of formula (A-III-2-B-2-a-i) or formula (A-III-2-B-2-a-ii):

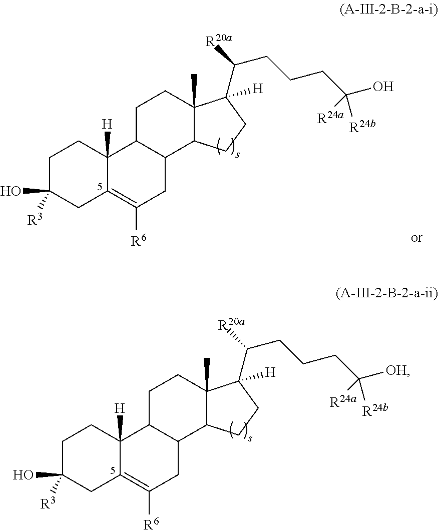

or a pharmaceutically acceptable salt thereof.

In some embodiments, the compound of formula (A-III-2-B-2-a-i) is a compound of formula (A-III-2-B-2-a-iii) or formula (A-III-2-B-2-a-iv):

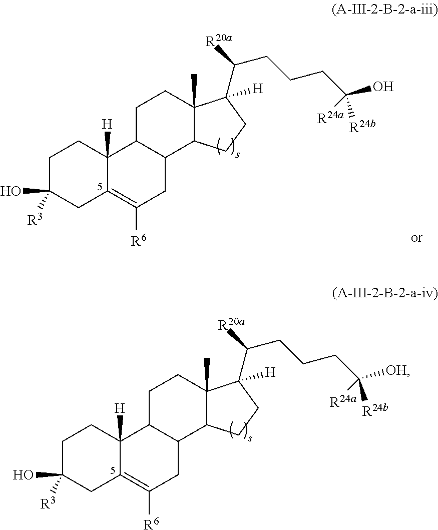

or a pharmaceutically acceptable salt thereof.

In some embodiments, the compound of formula (A-II-2-B-2-a-ii) is a compound of formula (A-III-2-B-2-a-v) or formula (A-III-2-B-2-a-vi):

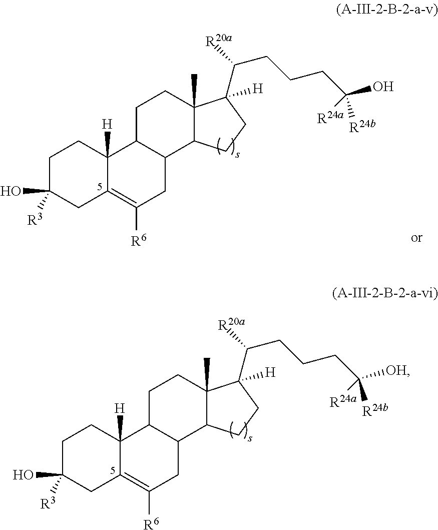

or a pharmaceutically acceptable salt thereof.

In some embodiments, the compound of formula (A-I) is a compound of formula (A-IV) or (A-V):

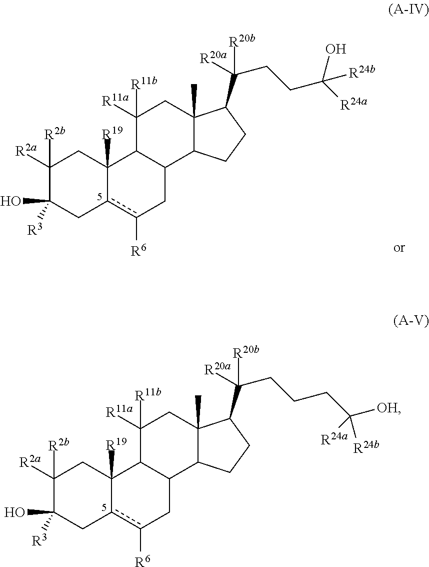

or a pharmaceutically acceptable salt thereof.

In some embodiments, the compound of formula (A-IV) is a compound of formula (A-IV-A) or (A-IV-B):

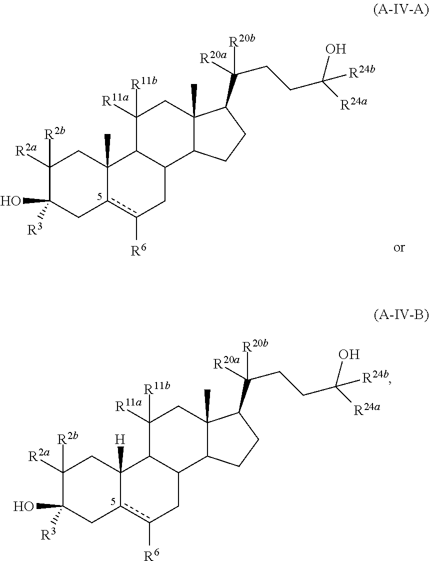

or a pharmaceutically acceptable salt thereof.

In some embodiments, the compound of formula (A-IV-A) is a compound of formula (A-IV-A-1) or (A-IV-A-2):

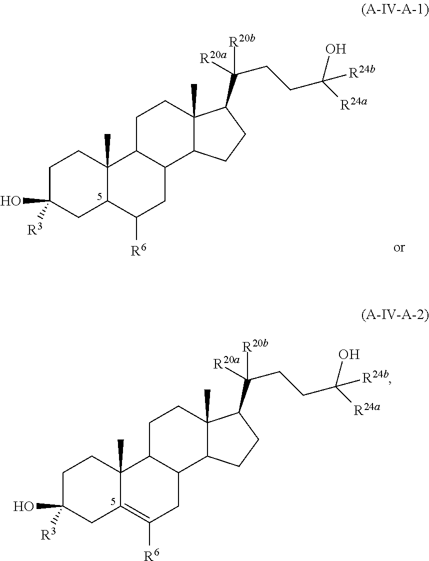

or a pharmaceutically acceptable salt thereof.

In some embodiments, the compound of formula (A-IV-A-1) is a compound of formula (A-IV-A-1-a):

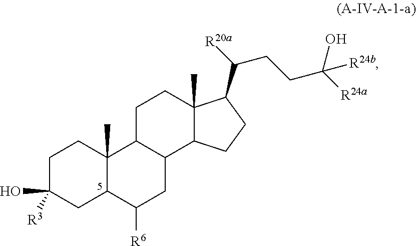

or a pharmaceutically acceptable salt thereof.

In some embodiments, the compound of formula (A-IV-A-1-a) is a compound of formula (A-IV-A-1-a-i) or (A-IV-A-1-a-ii):

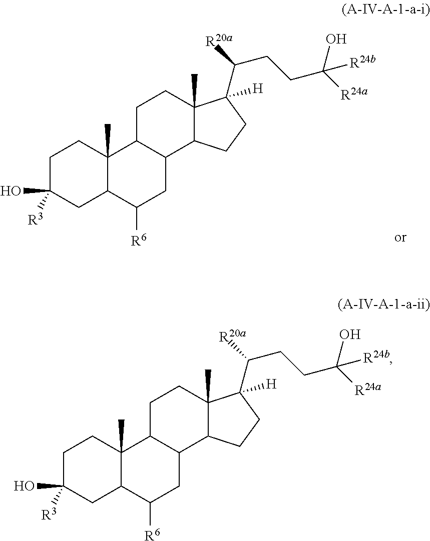

or a pharmaceutically acceptable salt thereof.

In some embodiments, the compound of formula (A-IV-A-1-a-i) is a compound of formula (A-IV-A-1-a-iii) or (A-IV-A-1-a-iv):

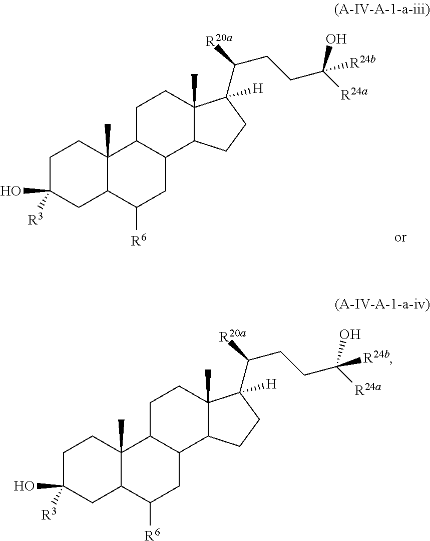

or a pharmaceutically acceptable salt thereof.

In some embodiments, the compound of formula (A-IV-A-1-a-ii) is a compound of formula (A-IV-A-1-a-v) or (A-IV-A-1-a-vi):

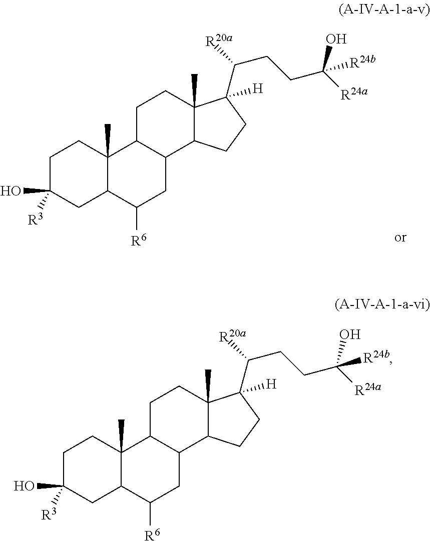

or a pharmaceutically acceptable salt thereof.

In some embodiments, the compound of formula (A-IV-A-2) is a compound of formula (A-IV-A-2-a):

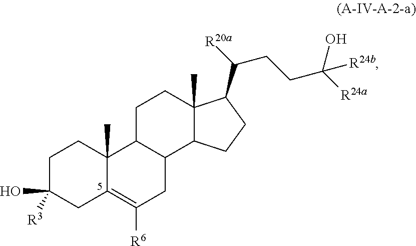

or a pharmaceutically acceptable salt thereof.

In some embodiments, the compound of formula (A-IV-A-2-a) is a compound of formula (A-IV-A-2-a-i) or (A-IV-A-2-a-ii):

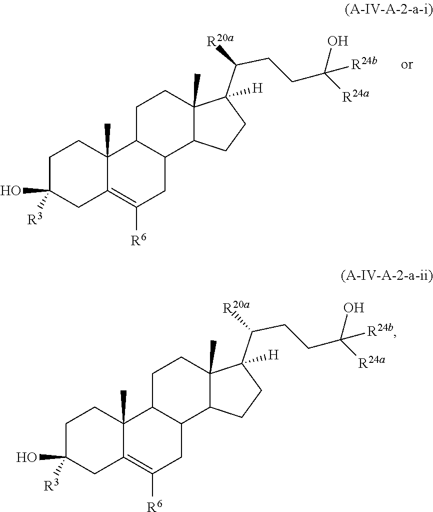

or a pharmaceutically acceptable salt thereof.

In some embodiments, formula (A-IV-A-2-a-i) is a compound of formula (A-IV-A-2-a-iii) or (A-IV-A-2-a-iv):

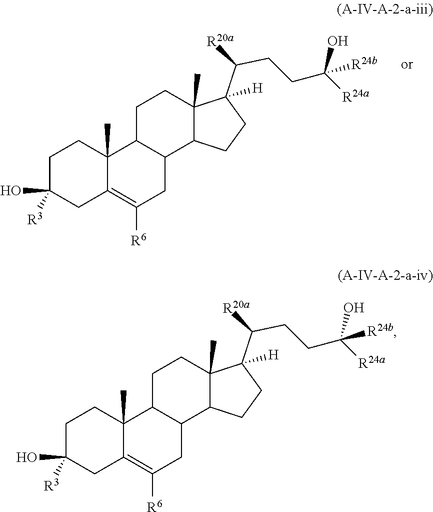

or a pharmaceutically acceptable salt thereof.

In some embodiments, the compound of formula (A-IV-A-2-a-ii) is a compound of formula (A-IV-A-2-a-v) or (A-IV-A-2-a-vi):

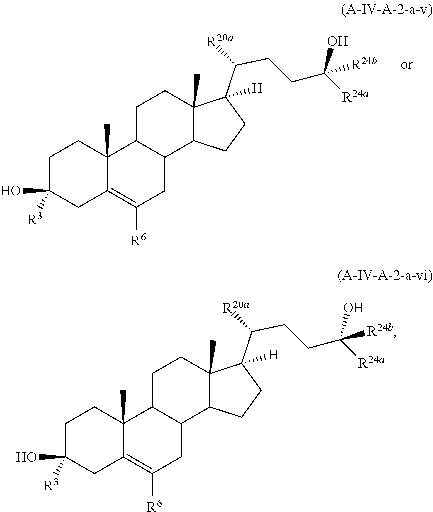

or a pharmaceutically acceptable salt thereof.

In some embodiments, the compound of formula (A-IV-B) is a compound of formula (A-IV-B-1) or (A-IV-B-2):

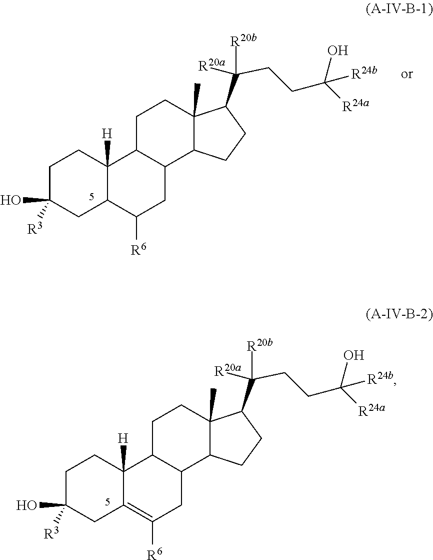

or a pharmaceutically acceptable salt thereof.

In some embodiments, the compound of formula (A-IV-B-1) is a compound of formula (A-IV-B-1-a):

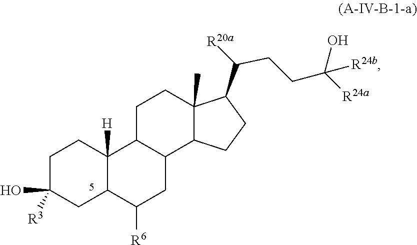

or a pharmaceutically acceptable salt thereof.

In some embodiments, the compound of formula (A-IV-B-1-a) is a compound of formula (A-IV-B-1-a-i) or (A-IV-B-1-a-ii):

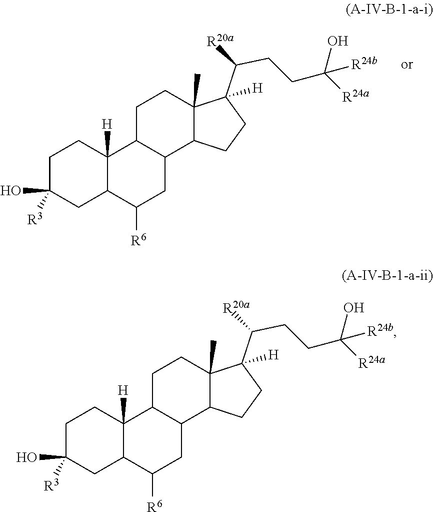

or a pharmaceutically acceptable salt thereof.

In some embodiments, the compound of formula (A-IV-B-1-a-i) is a compound of formula (A-IV-B-1-a-iii) or (A-IV-B-1-a-iv):

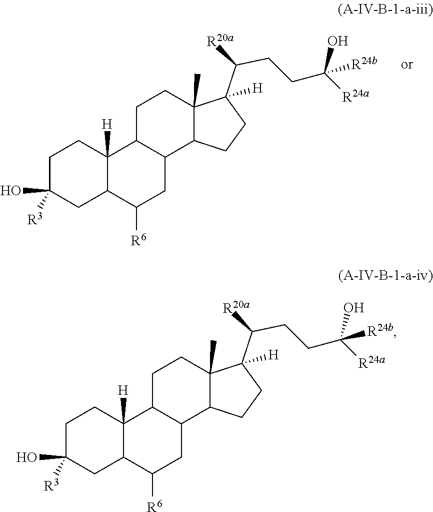

or a pharmaceutically acceptable salt thereof.

In some embodiments, formula (A-IV-B-1-a-ii) is a compound of formula (A-IV-B-1-a-v) or (A-IV-B-1-a-vi):

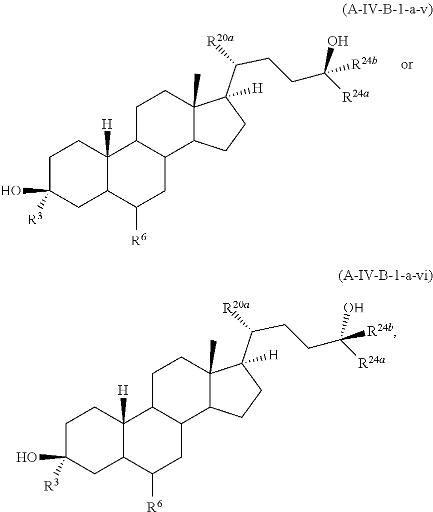

or a pharmaceutically acceptable salt thereof.

In some embodiments, the compound of formula (A-IV-B-2) is a compound of formula (A-IV-B-2-a):

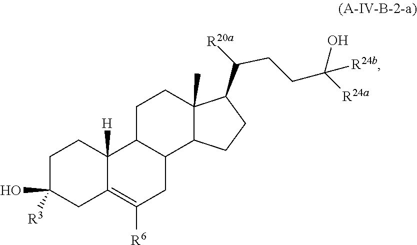

or a pharmaceutically acceptable salt thereof.

In some embodiments, the compound of formula (A-IV-B-2-a) is a compound of formula (A-IV-B-2-a-i) or (A-IV-B-2-a-ii):

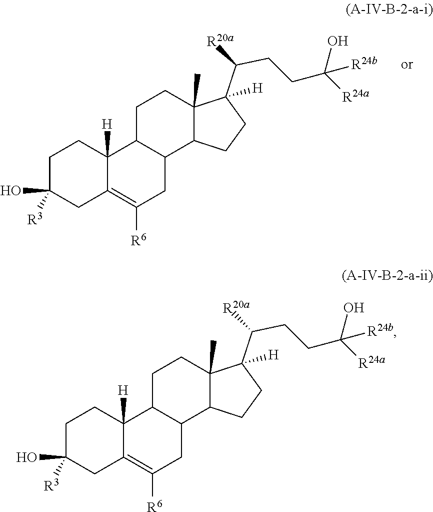

or a pharmaceutically acceptable salt thereof.

In some embodiments, the compound of formula (A-IV-B-2-a-i) is a compound of formula (A-IV-B-2-a-iii) or (A-IV-B-2-a-iv):

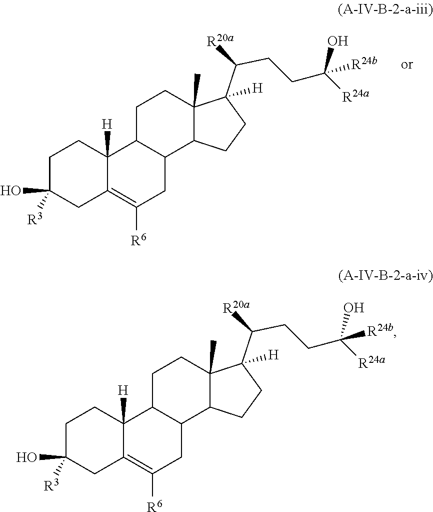

or a pharmaceutically acceptable salt thereof.

In some embodiments, the compound of formula (A-IV-B-2-a-ii) is a compound of formula (A-IV-B-2-a-v) or (A-IV-B-2-a-vi):

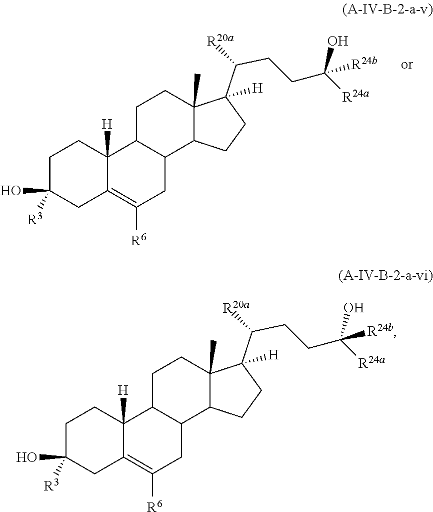

or a pharmaceutically acceptable salt thereof.

In some embodiments, the compound of formula (A-V) is a compound of formula (A-V-A) or (A-V-B):

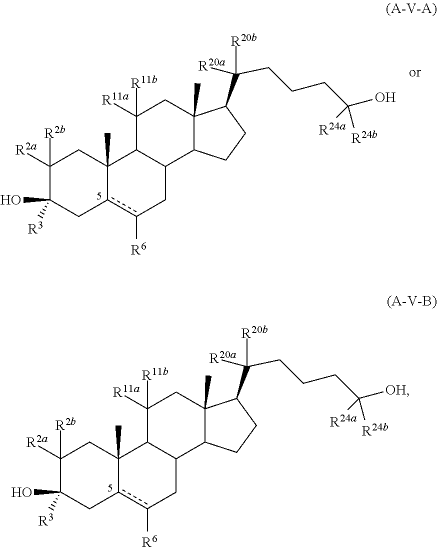

or a pharmaceutically acceptable salt thereof.

In some embodiments, the compound of formula (A-V-A) is a compound of formula (A-V-A-1) or (A-V-A-2):

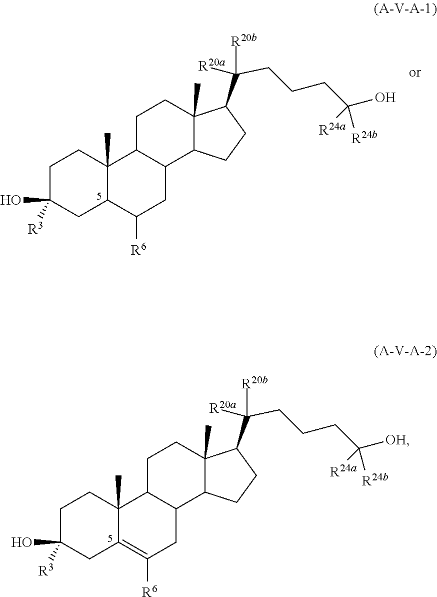

or a pharmaceutically acceptable salt thereof.

In some embodiments, the compound of formula (A-V-A-1) is a compound of formula (A-V-A-1-a):

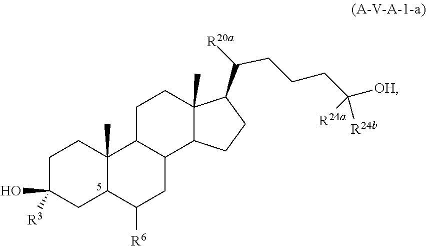

or a pharmaceutically acceptable salt thereof.

In some embodiments, the compound of formula (A-V-A-1-a) is a compound of formula (A-V-A-1-a-i) or (A-V-A-1-a-ii):

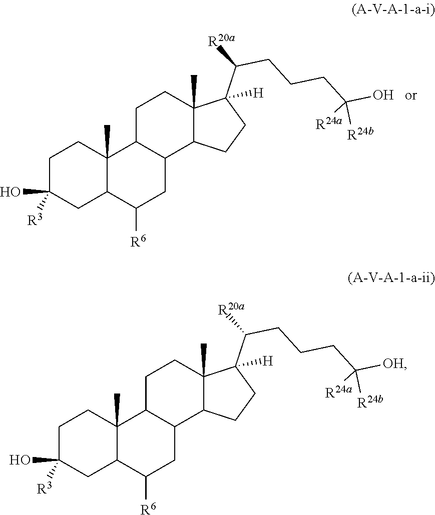

or a pharmaceutically acceptable salt thereof.

In some embodiments, the compound of formula (A-V-A-1-a-i) is a compound of formula (A-V-A-1-a-iii) or (A-V-A-1-a-iv):

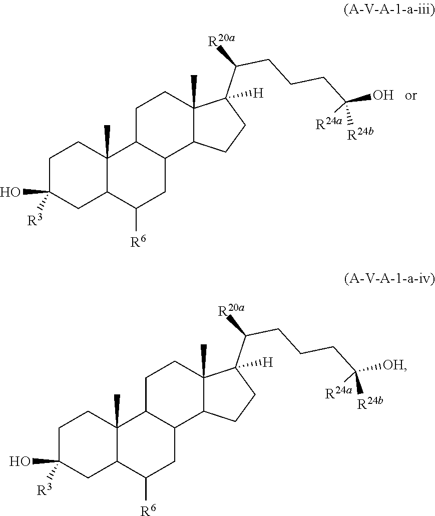

or a pharmaceutically acceptable salt thereof.

In some embodiments, the compound of formula (A-V-A-1-a-ii) is a compound of formula (A-V-A-1-a-v) or (A-V-A-1-a-vi):

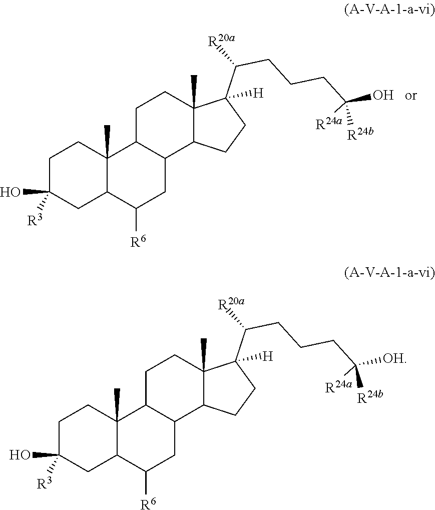

or a pharmaceutically acceptable salt thereof.

In some embodiments, the compound of formula (A-V-A-2) is a compound of formula (A-V-A-2-a):

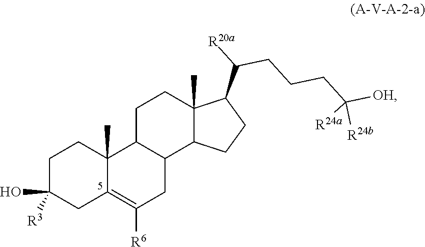

or a pharmaceutically acceptable salt thereof.

In some embodiments, the compound of formula (A-V-A-2-a) is a compound of formula (A-V-A-2-a-i) or (A-V-A-2-a-ii):

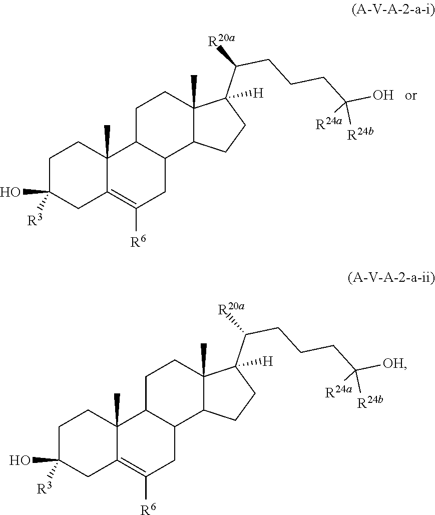

or a pharmaceutically acceptable salt thereof.

In some embodiments, the compound of formula (A-V-A-2-a-i) is a compound of formula (A-V-A-2-a-iii) or (A-V-A-2-a-iv):

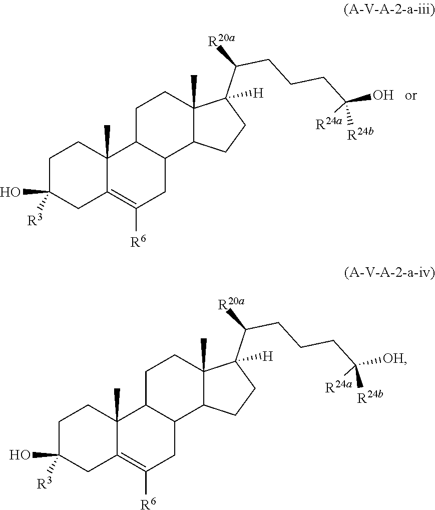

or a pharmaceutically acceptable salt thereof.

In some embodiments, the compound of formula (A-V-A-2-a-ii) is a compound of formula (A-V-A-2-a-v) or (A-V-A-2-a-vi):

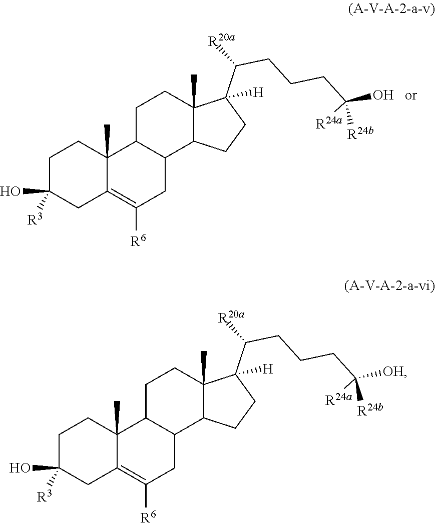

or a pharmaceutically acceptable salt thereof.

In some embodiments, the compound of formula (A-V-B) is a compound of formula (A-V-B-1) or (A-V-B-2):

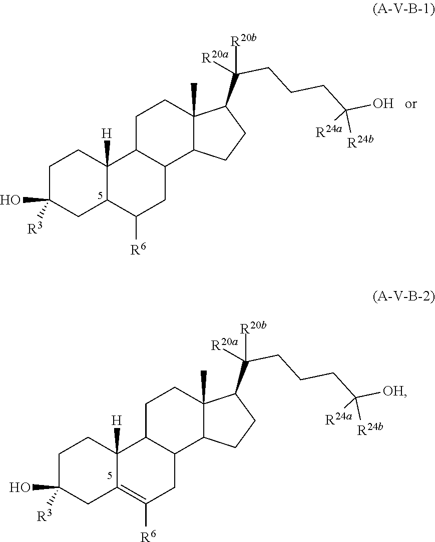

or a pharmaceutically acceptable salt thereof.

In some embodiments, the compound of formula (A-V-B-1) is a compound of formula (A-V-B-1-a):

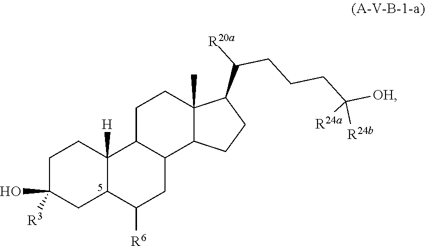

or a pharmaceutically acceptable salt thereof.

In some embodiments, the compound of formula (A-V-B-1-a) is a compound of formula (A-V-B-1-a-i) or (A-V-B-1-a-ii):

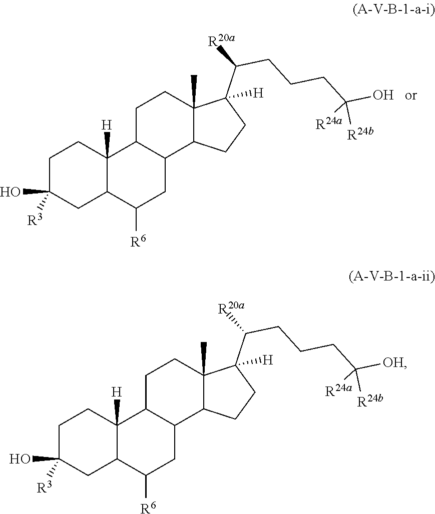

or a pharmaceutically acceptable salt thereof.

In some embodiments, the compound of formula (A-V-B-1-a-i) is a compound of formula (A-V-B-1-a-iii) or (A-V-B-1-a-iv):

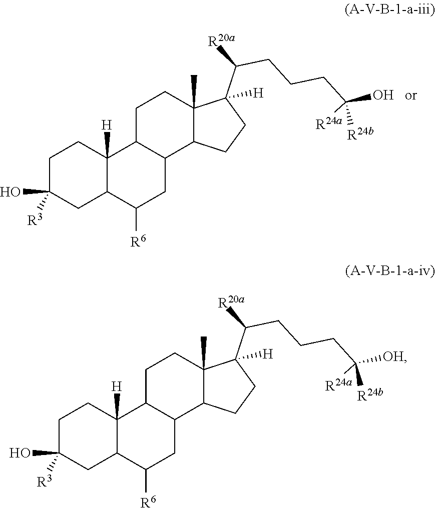

or a pharmaceutically acceptable salt thereof.

In some embodiments, the compound of formula (A-V-B-1-a-ii) is a compound of formula (A-V-B-1-a-v) or (A-V-B-1-a-vi):

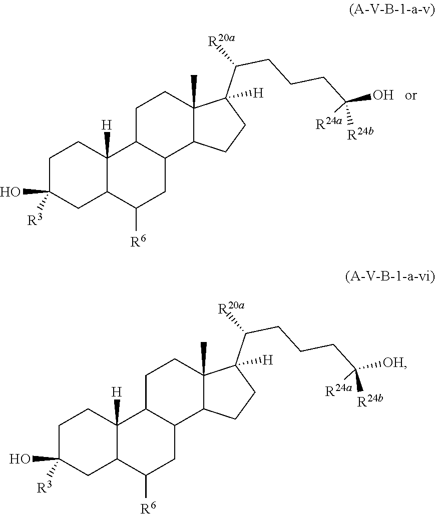

or a pharmaceutically acceptable salt thereof.

In some embodiments, the compound of formula (A-V-B-2) is a compound of formula (A-V-B-2-a):

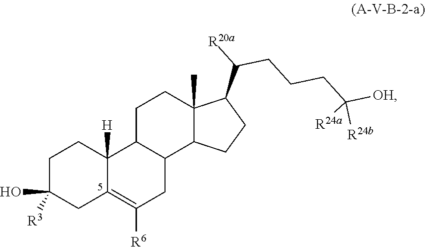

or a pharmaceutically acceptable salt thereof.

In some embodiments, the compound of formula (A-V-B-2-a) is a compound of formula (A-V-B-2-a-i) or (A-V-B-2-a-ii):

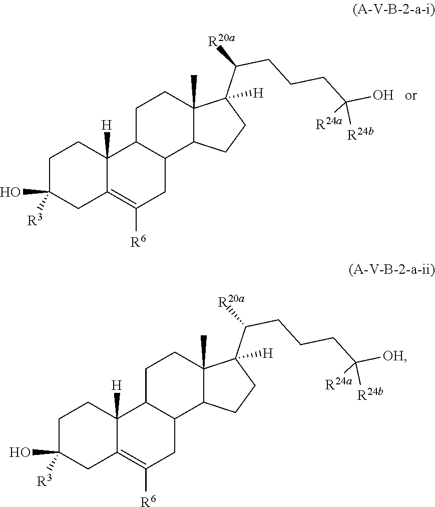

or a pharmaceutically acceptable salt thereof.

In some embodiments, the compound of formula (A-V-B-2-a-i) is a compound of formula (A-V-B-2-a-iii) or (A-V-B-2-a-iv):

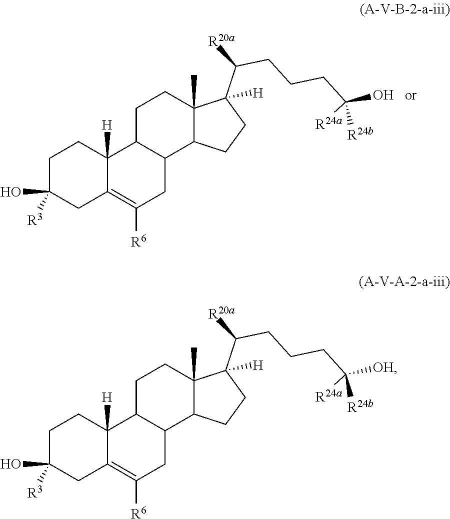

or a pharmaceutically acceptable salt thereof.

In some embodiments, the compound of formula (A-V-B-2-a-ii) is a compound of formula (A-V-B-2-a-v) or (A-V-B-2-a-vi):

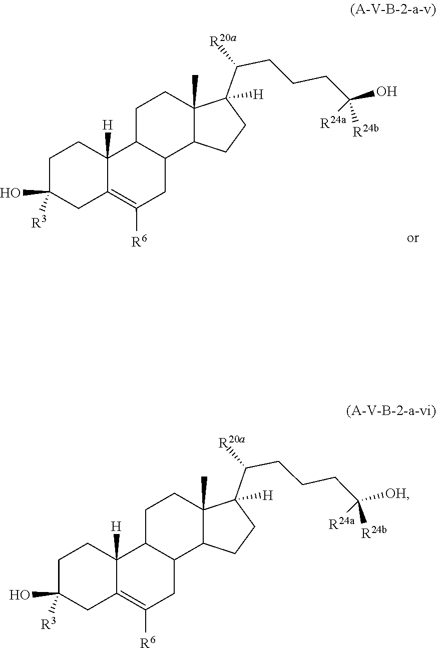

or a pharmaceutically acceptable salt thereof.

In some embodiments, the compound of formula (A-I) is a compound of formula (A-VI):

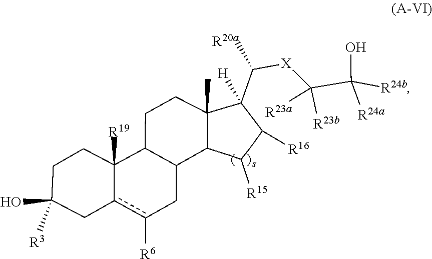

or a pharmaceutically acceptable salt thereof.

In some embodiments, the compound of formula (A-I) is a compound of formula (A-VII):

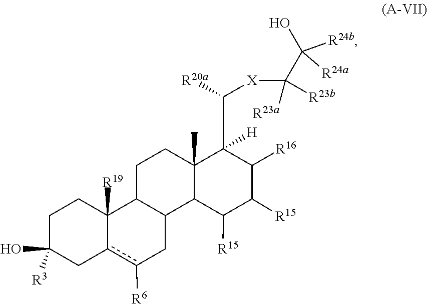

or a pharmaceutically acceptable salt thereof.

In some embodiments, the compound of formula (A-I) is a compound of formula (A-VIII):

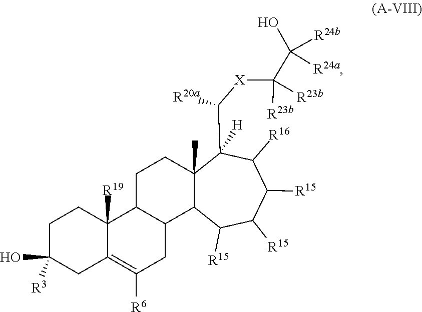

or a pharmaceutically acceptable salt thereof.

In some embodiments, the compound of formula (A-I) is a compound of formula (A-IX):

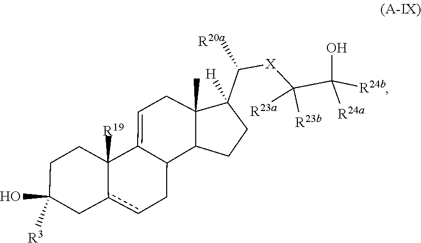

or a pharmaceutically acceptable salt thereof.

In some embodiments, the compound of formula (A-I) is a compound of formula (A-X):

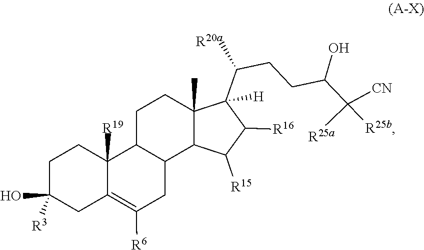

or a pharmaceutically acceptable salt thereof, wherein:
   R^{25a} is selected from the group consisting of substituted or unsubstituted C₁₋₆alkyl, substituted or unsubstituted C₂₋₆alkenyl, substituted or unsubstituted C₂₋₆ alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl;
   R^{25b} is hydrogen, substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, or substituted or unsubstituted 5- to 10-membered heteroaryl; or
   R^{25a} and R^{25b}, taken together with the carbon atom to which they are attached, form a substituted or unsubstituted C₃₋₆carbocyclyl; and
   R³, R⁶, R¹⁵, R¹⁶, R¹⁹, and R^{20a} are as defined herein.

In some embodiments, the compound of formula (A-I) is a compound of formula (A-XI):

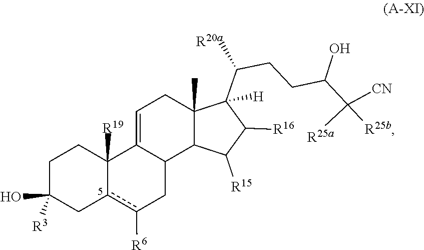

or a pharmaceutically acceptable salt thereof, wherein:
   R^{25a} is selected from the group consisting of substituted or unsubstituted C₁₋₆alkyl, substituted or unsubstituted C₂₋₆alkenyl, substituted or unsubstituted C₂₋₆ alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl;
   R^{25b} is hydrogen, substituted or unsubstituted C₁₋₆alkyl, substituted or unsubstituted C₂₋₆alkenyl, substituted or unsubstituted C₂₋₆alkynyl, substituted or unsubstituted
C₃₋₆carbocyclyl, substituted or unsubstituted 3- to 10-membered heterocyclyl, substituted or unsubstituted C₆₋₁₄ aryl, or substituted or unsubstituted 5- to 10-membered heteroaryl; or
   R^{25a} and R^{25b}, taken together with the carbon atom to which they are attached, form a substituted or unsubstituted C₃₋₆carbocyclyl; and
   R³, R⁶, R¹⁵, R¹⁶, R¹⁹, and R^{20a} are as defined herein.

In some embodiments, the compound of formula (A-I) is a compound of formula (A-XII):

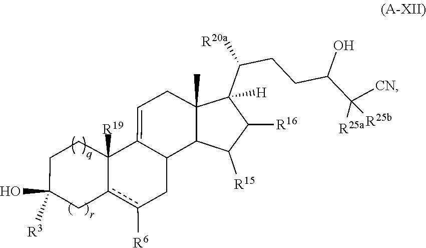

or a pharmaceutically acceptable salt thereof, wherein:
   R^{25a} is selected from the group consisting of substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl;
   R^{25b} is hydrogen, substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, or substituted or unsubstituted 5- to 10-membered heteroaryl; or
   R^{25a} and R^{25b}, taken together with the carbon atom to which they are attached, form a substituted or unsubstituted C₃₋₆carbocyclyl; and
   R³, R⁶, R¹⁵, R¹⁶, R¹⁹, and R^{20a} are as defined herein.

In some embodiments, the compound of formula (A-XII) is a compound of formula (A-XII-A) or (A-XII-B):

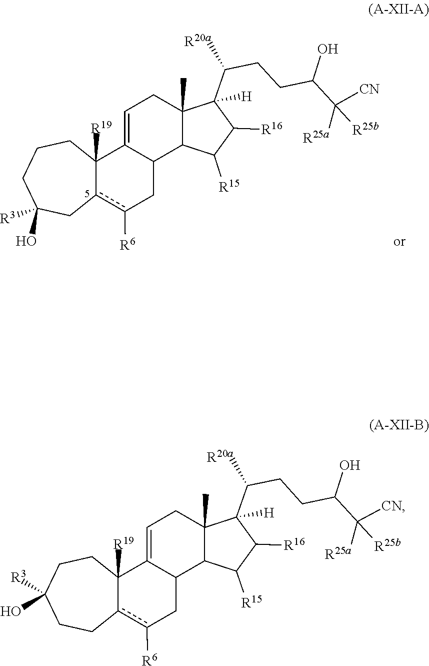

or a pharmaceutically acceptable salt thereof.

In some embodiments, the compound of formula (A-I) is a compound of formula (A-XIII):

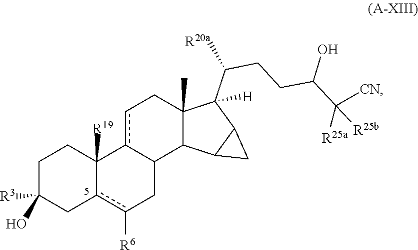

or a pharmaceutically acceptable salt thereof, wherein:
   R^{25a} is selected from the group consisting of substituted or unsubstituted C₁₋₆ alkyl, substituted or unsubstituted C₂₋₆alkenyl, substituted or unsubstituted C₂₋₆ alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl;
   R^{25b} is hydrogen, substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, or substituted or unsubstituted 5- to 10-membered heteroaryl; or
   R^{25a} and R^{25b}, taken together with the carbon atom to which they are attached, form a substituted or unsubstituted C₃₋₆carbocyclyl; and
   R³, R⁶, R¹⁹, and R^{20a} are as defined herein.
      Groups R^{2a} and R^{2b}—as they Relate to Formulae (A-I)-(A-XIII)

In certain embodiments, R^{2a} and R^{2b} are each independently hydrogen, halogen, substituted or unsubstituted C₁₋₆alkyl, substituted or unsubstituted C₃₋₁₀carbocyclyl, or OR^{A2}, wherein R^{A2} is hydrogen or substituted or unsubstituted C₁₋₆alkyl. In certain embodiments, each of R^{2a} and R^{2b} is hydrogen. In certain embodiments, each of R^{2a} and R^{2b} is independently halogen, e.g., fluoro, chloro, bromo, or iodo. In certain embodiments, each of R^{2a} and R^{2b} is independently fluoro or chloro. In certain embodiments, each of R^{2a} and R^{2b} is independently is substituted or unsubstituted C₁₋₆alkyl, e.g., substituted or unsubstituted C₁₋₂alkyl, substituted or unsubstituted C₂₋₃ alkyl, substituted or unsubstituted C₃₋₄ alkyl, substituted or unsubstituted C₄₋₅alkyl, or substituted or unsubstituted C₅₋₆alkyl. In certain embodiments, each of R^{2a} and R^{2b} is independently —CH₃, —CH₂CH₃, —CH₂CH₂CH₃, or cyclopropyl. In certain embodiments, each of R^{2a} and R^{2b} is independently —OR^{A2}. In certain embodiments, R^{A2} is hydrogen. In certain embodiments, R^{A2} is substituted or unsubstituted alkyl, e.g., substituted or unsubstituted C₁₋₆alkyl, substituted or unsubstituted C₁₋₂ alkyl, substituted or unsubstituted C₂₋₃alkyl, substituted or unsubstituted C₃₋₄ alkyl, substituted or unsubstituted C₄₋₅alkyl, or substituted or unsubstituted C₅₋₆alkyl. In certain embodiments, R^{A2} is hydrogen, —CH₃, —CH₂CH₃, or —CH₂CH₂CH₃, i.e., to provide a group of R^{2a} or R^{2b} each independently of formula-OH, —OCH₃, —OCH₂CH₃, or —OCH₂CH₂CH₃. In certain embodiments, one of R^{2a} and R^{2b} is a non-hydrogen substituent in the alpha configuration. In certain embodiments, one of R^{2a} and R^{2b} is a non-hydrogen substituent in the beta configuration.

In some embodiments, each of R^{2a} and R^{2b} is hydrogen.

### Group R³—as it Relates to Formulae (A-1)-(A-XIII)

In certain embodiments, R³ is substituted or unsubstituted aliphatic, i.e., substituted or unsubstituted alkyl, substituted or unsubstituted alkenyl, substituted or unsubstituted alkynyl, or substituted or unsubstituted carbocyclyl.

In certain embodiments, R³ is substituted or unsubstituted C₁₋₆alkyl, substituted or unsubstituted C₂₋₆alkenyl, substituted or unsubstituted C₂₋₆alkynyl, or substituted or unsubstituted C₃₋₁₀carbocyclyl.

In certain embodiments, R³ is substituted or unsubstituted alkyl, e.g., substituted or unsubstituted C₁₋₆alkyl, substituted or unsubstituted C₁₋₂ alkyl, substituted or unsubstituted C₂₋₃ alkyl, substituted or unsubstituted C₃₋₄ alkyl, substituted or unsubstituted C₄₋₅ alkyl, or substituted or unsubstituted C₅₋₆ alkyl. Exemplary R³ C₁₋₆alkyl groups include, but are not limited to, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), n-hexyl (C₆), C₁₋₆ alkyl substituted with 1, 2, 3, 4, 5, 6, 7, 8, 9, 10, or more fluoro groups (e.g., —CF₃, —CH₂F, —CHF₂, difluoroethyl, and 2,2,2-trifluoro-1,1-dimethyl-ethyl), C₁₋₆alkyl substituted with 1, 2, 3, 4, 5, 6, 7, 8, 9, 10, or more chloro groups (e.g., —CH₂Cl, —CHCl₂), and C₁₋₆alkyl substituted with alkoxy groups (e.g., —CH₂OCH₃, —CH₂OCH₂CH₃, —CH₂O-cyclopropyl). In certain embodiments, R³ is substituted C₁₋₆alkyl, e.g., R³ is C₁₋₆ haloalkyl, C₁₋₆alkoxyalkyl, or C₁₋₆ aminoalkyl. In certain embodiments, R³ is Me, Et, n-Pr, n-Bu, i-Bu, fluoromethyl, chloromethyl, difluoromethyl, trifluoromethyl, trifluoroethyl, difluoroethyl, 2,2,2-trifluoro-1,1-dimethyl-ethyl, methoxymethyl, methoxyethyl, or ethoxymethyl.

In certain embodiments, R³ is unsubstituted C₁₋₆alkyl. In some embodiments, R³ is —CH₃, —CH₂CH₃, or —CH₂CH₂CH₃. In some embodiments, R³ is —CH₂CH₃.

In certain embodiments, R³ is C₁₋₆alkyl substituted with one or more fluorine atoms; e.g., R³ is —CH₂F, —CHF₂, or —CF₃.

In certain embodiments, R³ is C₁₋₆alkyl substituted with one or more —OR^{A3} groups, wherein R^{A3} is hydrogen or substituted or unsubstituted alkyl. In certain embodiments, R³ is CH₂OR^{A3}, e.g., wherein R^{A3} is hydrogen, —CH₃, —CH₂CH₃, or —CH₂CH₂CH₃.

In certain embodiments, R³ is substituted or unsubstituted alkenyl, e.g., substituted or unsubstituted C₂₋₆alkenyl, substituted or unsubstituted C₂₋₃alkenyl, substituted or unsubstituted C₃₋₄ alkenyl, substituted or unsubstituted C₄₋₅alkenyl, or substituted or unsubstituted C₅₋₆ alkenyl. In certain embodiments, R³ is ethenyl (C₂), propenyl (C₃), or butenyl (C₄), unsubstituted or substituted with one or more substituents selected from the group consisting of alkyl, halo, haloalkyl, alkoxyalkyl, or hydroxyl. In certain embodiments, R³ is ethenyl, propenyl, or butenyl, unsubstituted or substituted with alkyl, halo, haloalkyl, alkoxyalkyl, or hydroxy. In certain embodiments, R³ is ethenyl.

In certain embodiments, R³ is substituted or unsubstituted alkynyl, e.g., substituted or unsubstituted C₂₋₆alkynyl, substituted or unsubstituted C₂₋₃ alkynyl, substituted or unsubstituted C₃₋₄alkynyl, substituted or unsubstituted C₄₋₅ alkynyl, or substituted or unsubstituted C₅₋₆ alkynyl. Exemplary substituted or unsubstituted R³ alkynyl groups include, but are not limited to, ethynyl, propynyl, or butynyl, unsubstituted or substituted with alkyl, halo, haloalkyl (e.g., CF₃), alkoxyalkyl, cycloalkyl (e.g., cyclopropyl or cyclobutyl), or hydroxyl. In certain embodiments, R³ is selected from the group consisting of trifluoroethynyl, cyclopropylethynyl, cyclobutylethynyl, and propynyl, fluoropropynyl, and chloroethynyl. In certain embodiments, R³ is ethynyl (C₂), propynyl (C₃), or butynyl (C₄), unsubstituted or substituted with one or more substituents selected from the group consisting of substituted or unsubstituted aryl, substituted or unsubstituted heteroaryl, substituted or unsubstituted carbocyclyl, and substituted or unsubstituted heterocyclyl. In certain embodiments, R³ is ethynyl (C₂), propynyl (C₃), or butynyl (C₄) substituted with substituted phenyl. In certain embodiments, the phenyl substituent is further substituted with one or more substituents selected from the group consisting of halo, alkyl, trifluoroalkyl, alkoxy, acyl, amino or amido. In certain embodiments, R³ is ethynyl (C₂), propynyl (C₃), or butynyl (C₄) substituted with substituted or unsubstituted pyrrolyl, imidazolyl, pyrazolyl, oxazoyl, thiazolyl, isoxazoyl, 1,2,3-triazolyl, 1,2,4-triazolyl, oxadiazolyl, thiadiazolyl, or tetrazolyl.

In certain embodiments, R³ is ethynyl, propynyl, or butynyl, unsubstituted or substituted with alkyl, halo, haloalkyl, alkoxyalkyl, or hydroxyl. In certain embodiments, R³ is ethynyl or propynyl, substituted with substituted or unsubstituted aryl. In certain embodiments, R³ is ethynyl or propynyl, substituted with phenyl unsubstituted or substituted with halo, alkyl, alkoxy, haloalkyl, trihaloalkyl, or acyl. In certain embodiments, R³ is ethynyl or propynyl, substituted with substituted or unsubstituted carbocyclyl. In certain embodiments, R³ is ethynyl or propynyl, substituted with substituted or unsubstituted cyclopropyl, cyclobutyl, cyclopentyl, or cyclohexyl. In certain embodiments, R³ is ethynyl or propynyl, substituted with substituted or unsubstituted heteroaryl. In certain embodiments, R³ is ethynyl or propynyl, substituted with substituted or unsubstituted pyridinyl, or pyrimidinyl. In certain embodiments, R³ is ethynyl or propynyl, substituted with substituted or unsubstituted pyrrolyl, imidazolyl, pyrazolyl, oxazoyl, thiazolyl, isoxazoyl, 1,2,3-triazolyl, 1,2,4-triazolyl, oxadiazolyl, thiadiazolyl, tetrazolyl. In certain embodiments, R³ is ethynyl or propynyl, substituted with substituted or unsubstituted heterocyclyl. In certain embodiments, R³ is ethynyl or propynyl, substituted with substituted or unsubstituted pyrrolidinyl, piperidinyl, piperazinyl, or mopholinyl. In certain embodiments, R³ is propynyl or butynyl, substituted with hydroxyl or alkoxy. In certain embodiments, R³ is propynyl or butynyl, substituted with methoxy or ethoxy. In certain embodiments, R³ is ethynyl or propynyl, substituted with chloro. In certain embodiments, R³ is ethynyl or propynyl, substituted with trifluoromethyl.

In certain embodiments, R³ is substituted or unsubstituted carbocyclyl, e.g., substituted or unsubstituted C₃₋₁₀carbocyclyl, substituted or unsubstituted C₃₋₆carbocyclyl, substituted or unsubstituted C₃₋₄ carbocyclyl, substituted or unsubstituted C₄₋₅ carbocyclyl, or substituted or unsubstituted C₅₋₆ carbocyclyl. In certain embodiments, R³ is substituted or unsubstituted cyclopropyl or substituted or unsubstituted cyclobutyl.

Groups R⁵ and R⁶—as they Relate to Formulae (A-I)-(A-XIII)

In some embodiments R⁵ is in the alpha or beta configuration. In certain embodiments R⁵ is in the alpha configuration. In certain embodiments R⁵ is in the beta configuration. In some embodiments, when the bond between C5 and C6 is a double bond, R⁵ is absent. In some embodiments, the bond between C5 and C6 is a single bond and R⁵ is hydrogen in the alpha configuration.

In certain embodiments, R⁵ is absent, hydrogen, halogen, or substituted or unsubstituted C₁₋₆alkyl. In some embodiments, R⁵ is halogen. In some embodiments, R⁵ is substituted C₁₋₆alkyl. In certain embodiments, R⁵ is substituted or unsubstituted alkyl, e.g., substituted or unsubstituted C₁₋₆alkyl, substituted or unsubstituted C₁₋₂ alkyl, substituted or unsubstituted C₂₋₃ alkyl, substituted or unsubstituted C₃₋₄ alkyl, substituted or unsubstituted C₄₋₅alkyl, or substituted or unsubstituted C₅₋₆ alkyl. In some embodiments, R⁵ is substituted or unsubstituted C₁₋₆alkyl. In some embodiments, R⁵ is unsubstituted C₁₋₆alkyl. In some embodiments, R⁵ is unsubstituted C₁ alkyl. In some embodiments, R⁵ is —CH₃.

In certain embodiments, R⁶ is hydrogen, halogen, or substituted or unsubstituted C₁₋₆alkyl. In certain embodiments, R⁶ is hydrogen. In certain embodiments, R⁶ is halogen, e.g., fluoro. In certain embodiments, R⁶ is substituted or unsubstituted alkyl, e.g., substituted or unsubstituted C₁₋₆alkyl, substituted or unsubstituted C₁₋₂ alkyl, substituted or unsubstituted C₂₋₃ alkyl, substituted or unsubstituted C₃₋₄ alkyl, substituted or unsubstituted C₄₋₅ alkyl, or substituted or unsubstituted C₅₋₆ alkyl. In certain embodiments, R⁶ is —CH₃ or —CF₃. In certain embodiments, R⁶ is hydrogen, —CH₃, or —F.

Groups R⁹, R^{11a}, and R^{11b}—as they Relate to Formulae (A-I)-(A-XIII)

In certain embodiments, R⁹ is absent or hydrogen. In some embodiments, R⁹ is hydrogen. In some embodiments, when the bond between C9 and C11 is a double bond, R⁹ is absent. In some embodiments, the bond between C9 and C11 is a double bond, R⁹ is absent and R^{11a} is selected from the group consisting of hydrogen, halogen, substituted or unsubstituted C₁₋₆alkyl, and —OR^{A11}, wherein R^{A11} is hydrogen or substituted or unsubstituted C₁₋₆alkyl, and R^{11b} is absent or hydrogen. In some embodiments, the bond between C9 and C11 is a single bond, R⁹ is hydrogen and R¹¹ª is selected from the group consisting of hydrogen, halogen or substituted or unsubstituted C₁₋₆alkyl or —OR^{A11}, wherein R^{A11} is hydrogen or substituted or unsubstituted C₁₋₆alkyl, and R^{11b} is absent or hydrogen.

In certain embodiments, R¹¹ª is hydrogen, halogen, substituted or unsubstituted C₁₋₆alkyl, or —OR^{A11}, wherein R^{A11} is hydrogen or substituted or unsubstituted C₁₋₆alkyl, and R^{11b} is absent or hydrogen; or R^{11a} and R^{11b} are joined to form an oxo (═O) group.

In certain embodiments, R^{11a} and R^{11b} are joined to form an oxo (═O) group.

In certain embodiments, R^{11a} is —OR^{A11} and R^{11b} is hydrogen. In certain embodiments, wherein R^{11a} is —OR^{A11}, R^{11a} is in the alpha or beta configuration. In certain embodiments, wherein R^{11a} is —OR^{A11}, R^{11a} is in the alpha configuration. In certain embodiments, wherein R^{11a} is —OR^{A11}, R^{11a} is in the beta configuration. In certain embodiments, R^{A11} is hydrogen. In certain embodiments, R^{A11} is substituted or unsubstituted alkyl, e.g., substituted or unsubstituted C₁₋₆alkyl, substituted or unsubstituted C₁₋₂ alkyl, substituted or unsubstituted C₂₋₃ alkyl, substituted or unsubstituted C₃₋₄alkyl, substituted or unsubstituted C₄₋₅alkyl, or substituted or unsubstituted C₅₋₆ alkyl. In certain embodiments, R^{A11} is hydrogen, —CH₃, —CH₂CH₃, or —CH₂CH₂CH₃, i.e., to provide a group R^{11a} of formula —OH, —OCH₃, —OCH₂CH₃, or —OCH₂CH₂CH₃.

In some embodiments, R^{11b} is absent or hydrogen. In some embodiments, R^{11b} is hydrogen. In some embodiments, the bond between C9 and C11 is a double bond, and R^{11b} is absent.

In some embodiments, each of R^{11a} and R^{11b} is hydrogen.

In some embodiments, R⁹ and R^{11a} are taken together with the carbon atoms to which they are attached to form a substituted or unsubstituted C₃₋₆carbocyclyl.

Groups R¹⁵ and R¹⁶—as they Relate to Formulae (A-I)-(A-XIII)

In certain embodiments, R¹⁵ is hydrogen, halogen, —OH, —NH₂, substituted or unsubstituted C₁₋₆alkyl, substituted or unsubstituted C₂₋₆alkenyl, substituted or unsubstituted C₂₋₆alkynyl, substituted or unsubstituted C₁₋₆alkoxy, substituted or unsubstituted C₃₋₆ carbocyclyl, or substituted or unsubstituted 3- to 10-membered heterocyclyl. In some embodiments, R¹⁵ is hydrogen, substituted or unsubstituted C₁₋₆alkyl or substituted or unsubstituted C₃₋₆carbocyclyl. In certain embodiments, R¹⁵ is hydrogen. In some embodiments, R¹⁵ is substituted or unsubstituted C₁₋₆alkyl. In some embodiments, R¹⁵ is unsubstituted C₁₋₆alkyl. In some embodiments, R¹⁵ is —CH₃. In some embodiments, R¹⁵ is substituted or unsubstituted C₃₋₆carbocyclyl. In some embodiments, R¹⁵ is substituted with substituted or unsubstituted cyclopropyl, cyclobutyl, cyclopentyl, or cyclohexyl. In some embodiments, R¹⁵ is unsubstituted cyclopropyl. In some embodiments, R¹⁵ is hydrogen, —CH₃ or cyclopropyl.

In certain embodiments, R¹⁶ is hydrogen, halogen, —OH, —NH₂, substituted or unsubstituted C₁₋₆alkyl, substituted or unsubstituted C₂₋₆alkenyl, substituted or unsubstituted C₂₋₆alkynyl, substituted or unsubstituted C₁₋₆alkoxy, substituted or unsubstituted C₃₋₆carbocyclyl, or substituted or unsubstituted 3- to 10-membered heterocyclyl. In some embodiments, R¹⁶ is hydrogen or unsubstituted C₁₋₆alkyl. In some embodiments, R¹⁶ is hydrogen or —CH₃. In some embodiments, R¹⁶ is hydrogen. In certain embodiments, R¹⁶ is substituted or unsubstituted C₁₋₆alkyl. In some embodiments, R¹⁶ is unsubstituted C₁₋₆alkyl. In some embodiments, R¹⁶ is —CH₃.

In certain embodiments, R¹⁵ and R¹⁶ are taken together with the carbon atoms to which they are attached to form a substituted or unsubstituted C₃-6 carbocyclyl. In some embodiments, R¹⁵ and R¹⁶ are taken together with the carbon atoms to which they are attached to form a substituted or unsubstituted cyclopropyl, cyclobutyl, cyclopentyl, or cyclohexyl. In some embodiments, R¹⁵ and R¹⁶ are taken together with the carbon atoms to which they are attached to form an unsubstituted cyclopropyl, cyclobutyl, cyclopentyl, or cyclohexyl. In some embodiments, R¹⁵ and R¹⁶ are taken together with the carbon atoms to which they are attached to form an unsubstituted cyclopropyl.

Groups R¹⁸ and R¹⁹—as they Relate to Formulae (A-I)-(A-XIII)

In some embodiments R¹⁸ is hydrogen, substituted or unsubstituted C₁₋₆alkyl, or OR^{A18}, wherein each instance of R^{A18} is independently substituted or unsubstituted C₁₋₆alkyl. In some embodiments, R¹⁸ is hydrogen, unsubstituted C₁₋₆alkyl, C₁₋₆alkyl substituted with OR^{A18}, or —OR^{A18}, wherein each instance of R^{A18} is independently substituted or unsubstituted C₁₋₆alkyl. In some embodiments, R¹⁸ is hydrogen, unsubstituted C₁₋₆alkyl, C₁₋₆alkyl substituted with OR^{A18}, or —OR^{A18}, wherein each instance of R^{A18} is independently unsubstituted C₁₋₆alkyl. In some embodiments, R¹⁸ is unsubstituted C₁₋₆alkyl. In some embodiments, R¹⁸ is —CH₃, —CH₂CH₃, or —CH(CH₃)₂. In some embodiments, R¹⁸ is —CH₃. In some embodiments, R¹⁸ is —CH₂CH₃, or —CH(CH₃)₂. In some embodiments, R¹⁸ is substituted C₁₋₆alkyl. In some embodiments, R¹⁸ is C₁₋₆alkyl substituted with one or more OR^{A18}, wherein each instance of R^{A18} is independently substituted or unsubstituted C₁₋₆alkyl. In some embodiments, R¹⁸ is C₁₋₆alkyl substituted with OR^{A18}, wherein R^{A18} is substituted or unsubstituted C₁₋₆alkyl. In some embodiments, R¹⁸—OR^{A18}, wherein R^{A18} is substituted or unsubstituted C₁₋₆alkyl. In some embodiments, R¹⁸ is hydrogen. In some embodiments, R¹⁸ is hydrogen, —CH₃, —CH₂OCH₃, —OCH₃, —CH₂CH₃, or —CH(CH₃)₂. In some embodiments, R¹⁸ is hydrogen, —CH₂OCH₃, —OCH₃, —CH₂CH₃, or —CH(CH₃)₂. In some embodiments, R¹⁸ is hydrogen or —CH₃. In some embodiments, R¹⁸ is —CH₂CH₃.

In some embodiments R¹⁹ is hydrogen, substituted or unsubstituted C₁₋₆alkyl, or OR^{A19}; wherein each instance of R^{A19} is independently substituted or unsubstituted C₁₋₆alkyl. In some embodiments, R¹⁹ is hydrogen, unsubstituted C₁₋₆alkyl, C₁₋₆alkyl substituted with OR^{A19}, or —OR^{A19}; wherein each instance of R^{A19} is independently substituted or unsubstituted C₁₋₆alkyl. In some embodiments, R¹⁹ is hydrogen, unsubstituted C₁₋₆alkyl, C₁₋₆alkyl substituted with OR^{A19}, or —OR^{A19}; wherein each instance of R^{A19} is independently unsubstituted C₁₋₆alkyl. In some embodiments, R¹⁹ is unsubstituted C₁₋₆alkyl. In some embodiments, R¹⁹ is —CH₃, —CH₂CH₃, or —CH(CH₃)₂. In some embodiments, R¹⁹ is —CH₃. In some embodiments, R¹⁹ is —CH₂CH₃. In some embodiments, R¹⁹ is —CH₂CH₃, or —CH(CH₃)₂. In some embodiments, R¹⁹ is substituted C₁₋₆alkyl. In some embodiments, R¹⁹ is C₁₋₆alkyl substituted with one or more OR^{A19}; wherein each instance of R^{A19} is independently substituted or unsubstituted C₁₋₆alkyl. In some embodiments, R¹⁹ is C₁₋₆alkyl substituted with OR^{A19}, wherein R^{A19} is substituted or unsubstituted C₁₋₆alkyl. In some embodiments, R¹⁹ is —OR^{A19}; wherein R^{A19} is substituted or unsubstituted C₁₋₆alkyl. In some embodiments, R¹⁹ is hydrogen, —CH₃, —CH₂OCH₃, —OCH₃, —CH₂CH₃, or —CH(CH₃)₂. In some embodiments, R¹⁹ is hydrogen, —CH₂OCH₃, —OCH₃, —CH₂CH₃, or —CH(CH₃)₂. In some embodiments, R¹⁹ is hydrogen, —CH₃, or —CH₂CH₃. In some embodiments R¹⁹ is hydrogen or —CH₃. In some embodiments, R¹⁹ is hydrogen.

Groups R^{20a} and R^{20b}—as they Relate to Formulae (A-I)-(A-XIII)

In certain embodiments, R^{20a} is halogen, cyano, substituted C₁₋₆alkyl, unsubstituted C₂₋₆alkyl, substituted or unsubstituted C₁₋₆alkoxy, or substituted or unsubstituted C₃₋₆ carbocyclyl.

In certain embodiments, R^{20b} is hydrogen, halogen, cyano, substituted C₁₋₆alkyl, unsubstituted C₁₋₆alkyl, substituted or unsubstituted C₁₋₆alkoxy, or substituted or unsubstituted C₃₋₆carbocyclyl.

In certain embodiments, R^{20a} and R^{20b} are both simultaneously —CH₃.

In certain embodiments, R^{20a} is halogen, cyano, substituted C₁₋₆alkyl, unsubstituted C₂₋₆alkyl, or substituted or unsubstituted C₁₋₆alkoxy.

In certain embodiments, R^{20a} is halogen. In certain embodiments, R^{20a} is —F.

In certain embodiments, R^{20a} is substituted C₁₋₆alkyl. In certain embodiments, R^{20a} is —CF₃.

In certain embodiments, R^{20a} is unsubstituted C₂₋₆alkyl. In certain embodiments, R^{20a} is —CH₂CH₃.

In certain embodiments, R^{20a} is unsubstituted C₁₋₆alkoxy. In certain embodiments, R^{20a} is —OCH₃ or —OCH₂CH₃.

In certain embodiments, R^{20a} is cyano.

In certain embodiments, R^{20b} is hydrogen, halogen, or substituted or unsubstituted C₁₋₆alkyl.

In certain embodiments, R^{20b} is halogen. In certain embodiments, R^{20b} is —F.

In certain embodiments, R^{20b} is substituted C₁₋₆alkyl. In certain embodiments, R^{20b} is —CF₃.

In certain embodiments, R^{20b} is unsubstituted C₁₋₆alkyl. In certain embodiments, R^{20b} is —CH₃, —CH₂CH₃ or —CH(CH₃)₂.

In certain embodiments, R^{20b} is unsubstituted C₁₋₆alkoxy. In certain embodiments, R^{20b} is —OCH₃.

In some embodiments, R^{20b} is hydrogen.

Groups X, n, R^{23a}, R^{23b}, R^{24a}, R^{24b}, R^{25a}, and R^{25b}—as they Relate to Formulae (A-I)-(A-XIII)

In certain embodiments, X is —(C(R^{X})₂)ₙ— or —O—, wherein R^{X} is hydrogen or fluorine, or one R^{X} group and R^{23b} are joined to form a double bond.

In certain embodiments, X is —O—. In certain embodiments, X is —CH₂—. In certain embodiments, X is —CF₂—.

In some embodiments, X is —(C(R^{X})₂—, wherein R^{X} is hydrogen or fluorine. In some embodiments, X is —CH₂—. In some embodiments, X is —CH₂CH₂—.

In certain embodiments, n is selected from 1, 2 or 3. In some embodiments, n is 1. In some embodiments, n is 2. In some embodiments, n is 3.

In certain embodiments, each of R^{23a} and R^{23b} is independently hydrogen or fluorine. In some embodiments, each of R^{23a} and R^{23b} is independently hydrogen. In some embodiments, each of R^{23a} and R^{23b} is independently fluorine. In certain embodiments, at least one of R^{23a} and R^{23b} is fluorine. In certain embodiments, R^{X} and R^{23b} are joined to form a double bond, e.g., cis or trans double bond.

In certain embodiments, R^{24a} is selected from the group consisting of substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl.

In certain embodiments, R^{24b} is hydrogen, substituted or unsubstituted C₁₋₆alkyl group, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, or substituted or unsubstituted 5- to 10-membered heteroaryl.

In some embodiments, R^{24a} is a substituted or unsubstituted C₁₋₆alkyl. In some embodiments, R^{24a} is —CH₃, —CH(CH₃)₂, or —CF₃.

In some embodiments, R^{24b} is hydrogen or substituted or unsubstituted C₁₋₆alkyl. In some embodiments, R^{24b} is hydrogen or unsubstituted C₁₋₆alkyl. In some embodiments, R^{24b} is hydrogen. In some embodiments, R^{24b} is —CH₃.

In some embodiments, R^{24a} is unsubstituted C₁₋₆alkyl and R^{24b} is hydrogen or unsubstituted C₁₋₆alkyl. In some embodiments, R^{24a} is unsubstituted C₁₋₆alkyl and R^{24b} is hydrogen or unsubstituted C₁₋₆alkyl. In some embodiments, R^{24a} is —CH₃ and R^{24b} is —CH₃. In some embodiments, R^{24a} is —CH₂CH₃ and R^{24b} is hydrogen.

In some embodiments, R^{24a} and R^{24b}, taken together with the carbon atom to which they are attached, form a substituted or unsubstituted C₃₋₆carbocyclyl or substituted or unsubstituted 3- to 10-membered heterocyclyl. In some embodiments, R^{24a} and R^{24b}, taken together with the carbon atom to which they are attached, form an unsubstituted C₃₋₆ carbocyclyl. In some embodiments, R^{24a} and R^{24b}, taken together with the carbon atom to which they are attached, form a cyclopropyl. In some embodiments, R^{24a} and R^{24b}, taken together with the carbon atom to which they are attached, form an unsubstituted 3- to 10-membered heterocyclyl. In some embodiments, R^{24a} and R^{24b}, taken together with the carbon atom to which they are attached, form a substituted 3- to 10-membered heterocyclyl. In some embodiments, R^{24a} and R^{24b}, taken together with the carbon atom to which they are attached, form a substituted or unsubstituted 5- to 6-membered heterocyclyl. In some embodiments, R^{24a} and R^{24b}, taken together with the carbon atom to which they are attached, form a substituted or unsubstituted tetrahydrofuranyl, substituted or unsubstituted tetrahydropyranyl, substituted or unsubstituted piperidinyl, or substituted or unsubstituted morpholinyl. In some embodiments, R^{24a} and R^{24b}, taken together with the carbon atom to which they are attached, form a substituted tetrahydrofuranyl. In some embodiments, R^{24a} and R^{24b}, taken together with the carbon atom to which they are attached, form an unsubstituted tetrahydrofuranyl. In some embodiments, R^{24a} and R^{24b}, taken together with the carbon atom to which they are attached, form a substituted tetrahydropyranyl. In some embodiments, R^{24a} and R^{24b}, taken together with the carbon atom to which they are attached, form an unsubstituted tetrahydropyranyl. In some embodiments, R^{24a} and R^{24b}, taken together with the carbon atom to which they are attached, form a substituted piperidinyl. In some embodiments, R^{24a} and R^{24b}, taken together with the carbon atom to which they are attached, form an unsubstituted piperidinyl.

In some embodiments, R^{25a} is selected from the group consisting of substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl.

In some embodiments, R^{25b} is hydrogen, substituted or unsubstituted C₁₋₆alkyl group, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, or substituted or unsubstituted 5- to 10-membered heteroaryl.

In some embodiments, R^{25a} is a substituted or unsubstituted C₁₋₆alkyl. In some embodiments, R^{25a} is unsubstituted C₁₋₆alkyl. In some embodiments, R^{25a} is —CH₃, or CH₂CH₃.

In some embodiments, R^{25b} is hydrogen or substituted or unsubstituted C₁₋₆alkyl. In some embodiments, R^{25b} is hydrogen or unsubstituted C₁₋₆alkyl. In some embodiments, R^{25b} is hydrogen. In some embodiments, R^{25b} is —CH₃.

In some embodiments, R^{25a} is unsubstituted C₁₋₆alkyl and R^{25b} is hydrogen or unsubstituted C₁₋₆alkyl. In some embodiments, R^{25a} is unsubstituted C₁₋₆alkyl and R^{25b} is hydrogen or unsubstituted C₁₋₆alkyl. In some embodiments, R^{25a} is —CH₃ and R^{25b} is —CH₃. In some embodiments, R^{25a} is —CH₂CH₃ and R^{25b} is hydrogen.

In some embodiments, R^{25a} and R^{25b}, taken together with the carbon atom to which they are attached, form a substituted or unsubstituted C₃₋₆carbocyclyl. In some embodiments, R^{25a} and R^{25b} taken together with the carbon atom to which they are attached, form an unsubstituted C₃₋₆carbocyclyl. In some embodiments, R^{25a} and R^{25b} taken together with the carbon atom to which they are attached, form a cyclopropyl.

q, r, and s—as they Relate to Formulae (A-I)-(A-XIII)

In some embodiments, q is 0, 1 or 2. In some embodiments, q is 0. In some embodiments, q is 1. In some embodiments, q is 2.

In some embodiments, r is 1 or 2. In some embodiments, r is 1. In some embodiments, r is 2.

In some embodiments, s is 1, 2, or 3. In some embodiments, s is 1. In some embodiments, s is 2. In some embodiments, s is 3.

In some embodiments, q is 0 and r is 1. In some embodiments, q is 2 and r is 1. In some embodiments, q, r and s are each independently 1.

In some embodiments, the compound of Formula (A-I) is any one of Compounds 1-26 in Table A-1 or a pharmaceutically acceptable salt thereof. In some embodiments, the compound of Formula (A-I) is any one of Compounds 1-26 in Table A-1. In some embodiments, the compound of Formula (A-I) is a pharmaceutically acceptable salt of any one of Compounds 1-26 in Table A-1. In some embodiments, the compound of Formula (A-I) is any one of Compounds A-3, A-4, A-5, A-6, A-7, A-9, A-13, A-14, A-15, or A-16, or a pharmaceutically acceptable salt thereof. In some embodiments, the compound of Formula (A-I) is any one of Compounds A-3, A-4, A-5, A-6, A-7, A-9, A-13, A-14, A-15, or A-16. In some embodiments, the compound of Formula (A-I) is a pharmaceutically acceptable salt of any one of Compounds A-3, A-4, A-5, A-6, A-7, A-9, A-13, A-14, A-15, or A-16.

TABLE A-1

| Cmpd | Structure |
|---|---|
| A-1 | 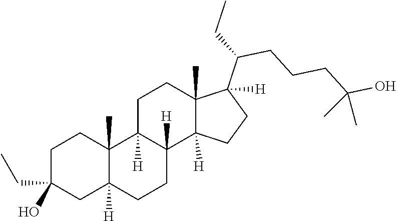 |
| A-2 | 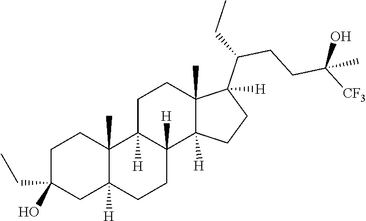 |
| A-3 | 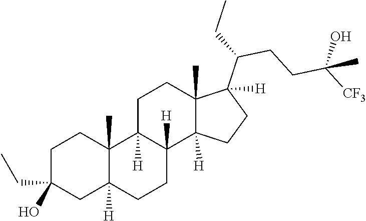 |
| A-4 | 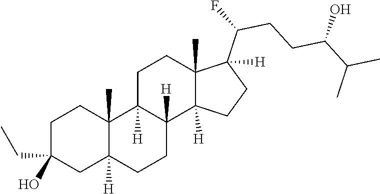 |
| A-5 | 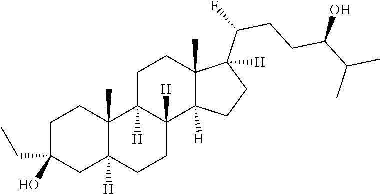 |
| A-6 | 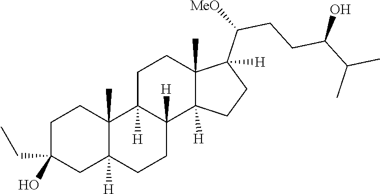 |
| A-7 | 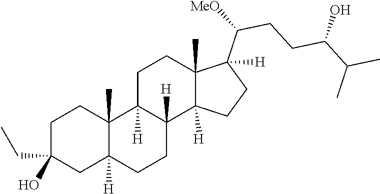 |
| A-8 | 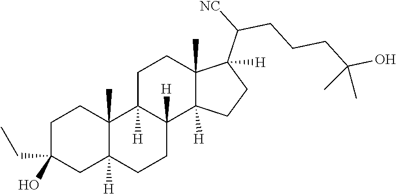 |
| A-9 | 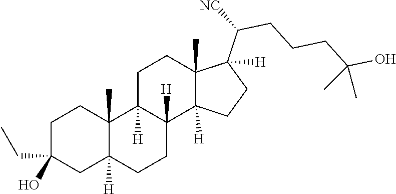 |
| A-9a | 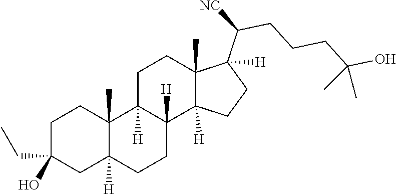 |
| A-10 | 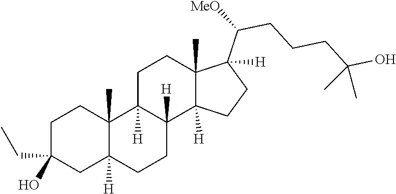 |
| A-11 | 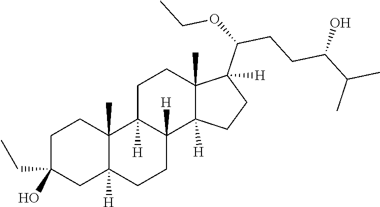 |
| A-11a | 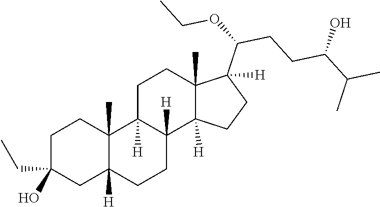 |
| A-12 | 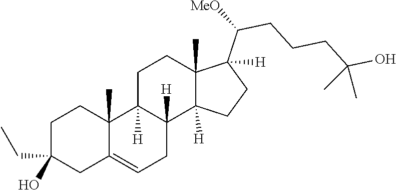 |
| A-13 | 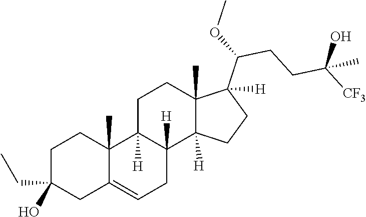 |
| A-14 | 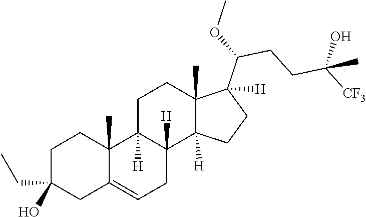 |
| A-15 | 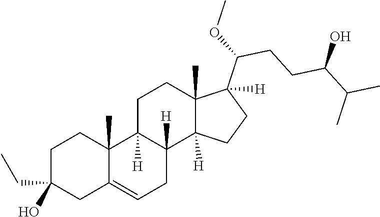 |
| A-16 | 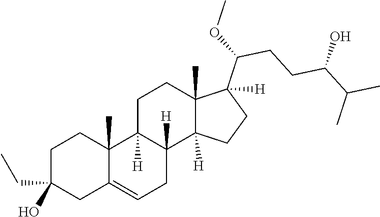 |
| A-17 | 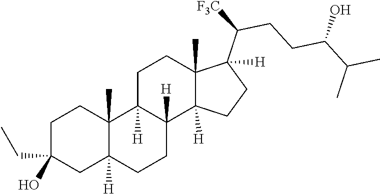 |
| A-18 | 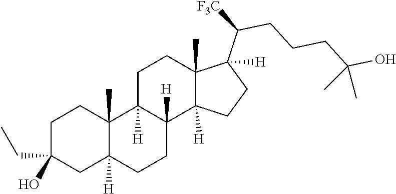 |
| A-19 | 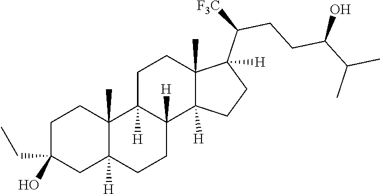 |
| A-20 | 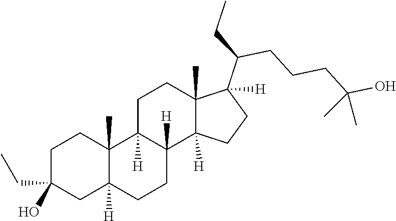 |
| A-21 | 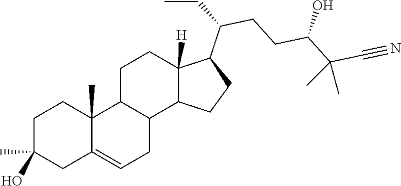 |
| A-22 | 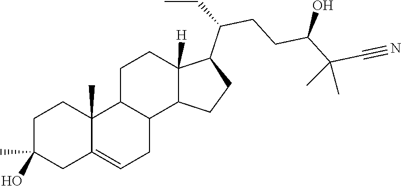 |
| A-23 | 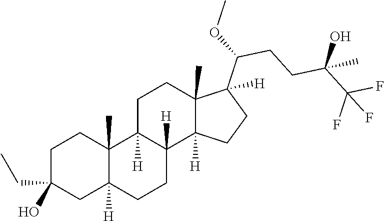 |
| A-24 | 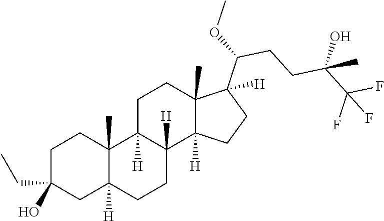 |
| A-25 | 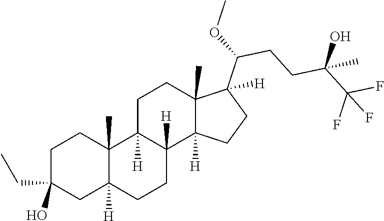 |
| A-26 | 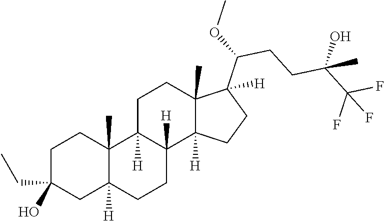 |

In one aspect, the present disclosure provides a compound of Formula (B-I):

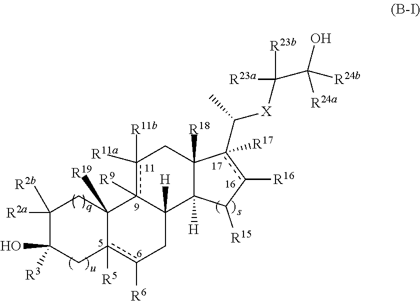

or a pharmaceutically acceptable salt thereof;
wherein:
   each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein R^{A2} is hydrogen or substituted or unsubstituted C₁₋₆alkyl;
   R³ is substituted or unsubstituted C₁₋₆alkyl, substituted or unsubstituted C₂₋₆alkenyl, substituted or unsubstituted C₂₋₆alkynyl, or substituted or unsubstituted C₃₋₁₀carbocyclyl;
   R⁵ is absent, hydrogen, halogen, or substituted or unsubstituted C₁₋₆alkyl;
   R⁶ is hydrogen, substituted or unsubstituted C₁₋₆alkyl, or halogen;
   R⁹ is absent or hydrogen; or
   R⁹ and R^{11a} are taken together with the carbon atoms to which they are attached to form a substituted or unsubstituted C₃₋₆carbocyclyl;
   R^{11a} is hydrogen, halogen, substituted or unsubstituted C₁₋₆alkyl, or —OR^{A11}, wherein R^{A11} is hydrogen or substituted or unsubstituted C₁₋₆alkyl;
   R^{11b} is absent or hydrogen; or
   R^{11a} and R^{11b} are joined to form an oxo (═O) group;
   R¹⁵ is hydrogen, halogen, —OH, —NH₂, substituted or unsubstituted C₁₋₆alkyl, substituted or unsubstituted C₂₋₆alkenyl, substituted or unsubstituted C₂₋₆alkynyl, substituted or unsubstituted C₁₋₆alkoxy, substituted or unsubstituted C₃₋₆carbocyclyl or substituted or unsubstituted 3- to 10-membered heterocyclyl;
   R¹⁶ is hydrogen, halogen —OH, —NH₂, substituted or unsubstituted C₁₋₆alkyl, substituted or unsubstituted C₂₋₆alkenyl, substituted or unsubstituted C₂₋₆alkynyl, substituted or unsubstituted C₁₋₆alkoxy, substituted or unsubstituted C₃₋₆carbocyclyl or substituted or unsubstituted 3- to 10-membered heterocyclyl;
   R¹⁷ is absent, hydrogen or substituted or unsubstituted C₁₋₆alkyl; provided that when the bond between C16 and C17 is a double bond, then R¹⁷ is absent;
   or R¹⁵ and R¹⁶ are taken together with the carbon atoms to which they are attached, to form a substituted or unsubstituted C₃₋₆carbocyclyl;
   or R¹⁶ and R¹⁷ are taken together with the carbon atoms to which they are attached to form a substituted or unsubstituted C₃₋₆carbocyclyl;
   provided that R¹⁵ and R¹⁶ and R¹⁶ and R¹⁷ cannot both be taken together with the carbon atoms to which they are attached, to form a substituted or unsubstituted C₃₋₆ carbocyclyl; and
   provided that at least one of R¹⁵, R¹⁶, and R¹⁷ is not hydrogen;
   R¹⁸ is hydrogen, substituted or unsubstituted C₁₋₆alkyl, OR^{A18}, wherein R^{A18} is substituted or unsubstituted C₁₋₆alkyl;
   R¹⁹ is hydrogen, substituted or unsubstituted C₁₋₆alkyl, OR^{A19}, wherein R^{A19} is substituted or unsubstituted C₁₋₆alkyl;
   X is —(C(R^{X})₂)ₙ— or —O—, wherein R^{X} is hydrogen or fluorine, or one R^{X} group and R^{23b} are joined to form a double bond;
   n is 1, 2 or 3;
   each instance of R^{23a} and R^{23b} is independently hydrogen or fluorine;
   R^{24a} is selected from the group consisting of substituted or unsubstituted C₁₋₆alkyl, substituted or unsubstituted C₂₋₆alkenyl, substituted or unsubstituted C₂₋₆ alkynyl, substituted or unsubstituted C₃₋₁₀carbocyclyl, substituted or unsubstituted 3- to 10-membered heterocyclyl, substituted or unsubstituted C₆₋₁₄ aryl, and substituted or unsubstituted 5- to 10-membered heteroaryl;
   R^{24b} is hydrogen or substituted or unsubstituted C₁₋₆alkyl;
   or R^{24a} and R^{24b}, taken together with the carbon atom to which they are attached, form a substituted or unsubstituted C₃₋₆carbocyclyl, or substituted or unsubstituted 3- to 10-membered heterocyclyl;
   q is 0, 1 or 2;
   u is 1 or 2;
   s is 1 or 2; and
   each [symbol] independently represents a single or double bond, provided if the bond between C5 and C6 is a double bond, R⁵ is absent, and if the bond between C9 and C11 is a double bond, R⁹ and R^{11b} are absent.

In one aspect, the present disclosure provides a compound of Formula (B-IA):

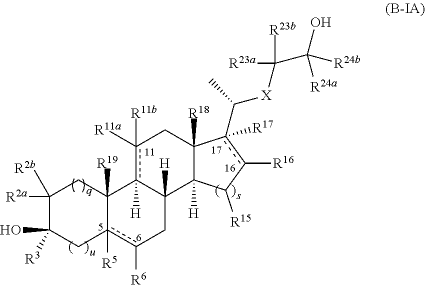

or a pharmaceutically acceptable salt thereof;
wherein:
   each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein R^{A2} is hydrogen or substituted or unsubstituted C₁₋₆alkyl;
   R³ is substituted or unsubstituted C₁₋₆alkyl, substituted or unsubstituted C₂₋₆alkenyl, substituted or unsubstituted C₂₋₆alkynyl, or substituted or unsubstituted C₃₋₁₀carbocyclyl;
   R⁶ is hydrogen, substituted or unsubstituted C₁₋₆alkyl, or halogen;
   R^{11a} is hydrogen or —OR^{A11}, wherein R^{A11} is hydrogen or substituted or unsubstituted C₁₋₆alkyl, and R^{11b} is hydrogen; or R^{11a} and R^{11b} are joined to form an oxo (═O) group;
   [symbol] represents a single or double bond; provided if the bond between C5 and C6 is a single bond, then the hydrogen at C5 is in the alpha configuration;
   R¹⁵ is hydrogen, halogen, —OH, —NH₂, substituted or unsubstituted C₁₋₆alkyl, substituted or unsubstituted C₂₋₆alkenyl, substituted or unsubstituted C₂₋₆alkynyl, substituted or unsubstituted C₁₋₆alkoxy, substituted or unsubstituted C₃₋₆carbocyclyl or substituted or unsubstituted 3- to 10-membered heterocyclyl;
   R¹⁶ is hydrogen, halogen —OH, —NH₂, substituted or unsubstituted C₁₋₆alkyl, substituted or unsubstituted C₂₋₆alkenyl, substituted or unsubstituted C₂₋₆alkynyl, substituted or unsubstituted C₁₋₆alkoxy, substituted or unsubstituted C₃₋₆carbocyclyl or substituted or unsubstituted 3- to 10-membered heterocyclyl;
   R¹⁷ is absent, hydrogen or substituted or unsubstituted C₁₋₆alkyl; provided that when the bond between C16 and C17 is a double bond, then R¹⁷ is absent;
   or R¹⁵ and R¹⁶ are taken together with the carbon atoms to which they are attached, to form a substituted or unsubstituted C₃₋₆carbocyclyl;
   or R¹⁶ and R¹⁷ are taken together with the carbon atoms to which they are attached to form a substituted or unsubstituted C₃₋₆carbocyclyl;
   provided that R¹⁵ and R¹⁶ and R¹⁶ and R¹⁷ cannot both be taken together with the carbon atoms to which they are attached, to form a substituted or unsubstituted C₃₋₆carbocyclyl; and
   provided that at least one of R¹⁵, R¹⁶, and R¹⁷ is not hydrogen;
   R¹⁸ is hydrogen, substituted or unsubstituted C₁₋₆alkyl, OR^{A18}, wherein R^{A18} is substituted or unsubstituted C₁₋₆alkyl;
   R¹⁹ is hydrogen, substituted or unsubstituted C₁₋₆alkyl, OR^{A19}, wherein R^{A19} is substituted or unsubstituted C₁₋₆alkyl;
   X is —(C(R^{X})₂)ₙ— or —O—, wherein R^{X} is hydrogen or fluorine, wherein n 1, 2 or 3;
   each instance of R^{23a} and R^{23b} is independently hydrogen or fluorine, or one R^{X} group and R^{23b} are joined to form a double bond;
   R^{24a} is a non-hydrogen group selected from the group consisting of substituted or unsubstituted C₁₋₆alkyl, substituted or unsubstituted C₂₋₆alkenyl, substituted or unsubstituted C₂₋₆alkynyl, substituted or unsubstituted C₃₋₁₀carbocyclyl, substituted or unsubstituted 3- to 10-membered heterocyclyl, substituted or unsubstituted C₆₋₁₄ aryl, and substituted or unsubstituted 5- to 10-membered heteroaryl;
   R^{24b} is hydrogen or a substituted or unsubstituted C₁₋₆alkyl;
   or R^{24a} and R^{24b}, taken together with the carbon atom to which they are attached, form a substituted or unsubstituted C₃₋₆carbocyclyl, or substituted or unsubstituted 3- to 10-membered heterocyclyl;
   q is 0, 1 or 2;
   u is 1 or 2; and
   s is 1 or 2.

In some embodiments, the compound of formula (B-I) is a compound of formula (B-I-a):

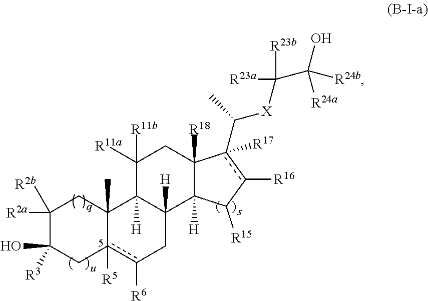

or a pharmaceutically acceptable salt thereof.

In some embodiments, the compound of formula (B-I) is a compound of formula (B-I-b):

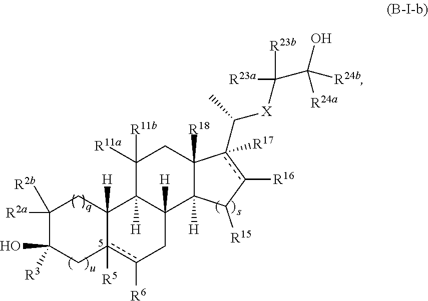

or a pharmaceutically acceptable salt thereof.

In some embodiments, the compound of formula (B-I) is a compound of formula (B-II):

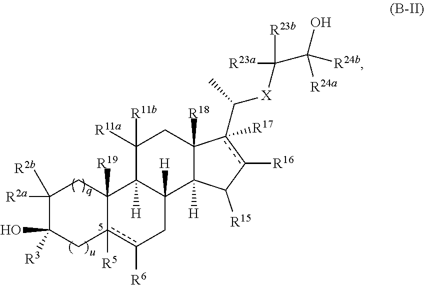

or a pharmaceutically acceptable salt thereof.

In some embodiments, the compound of formula (B-II) is a compound of formula (B-IIa):

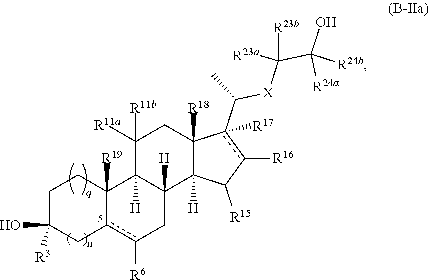

or a pharmaceutically acceptable salt thereof.

In some embodiments, the compound of formula (B-IIa) is a compound of formula (B-IIa-1):

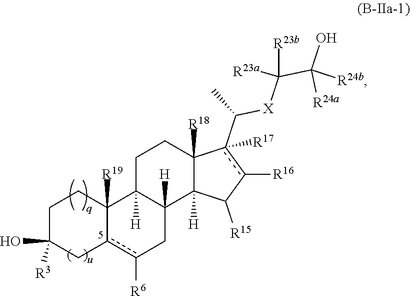

or a pharmaceutically acceptable salt thereof.

In some embodiments, the compound of formula (B-IIa-1) is a compound of formula (B-IIa-1a):

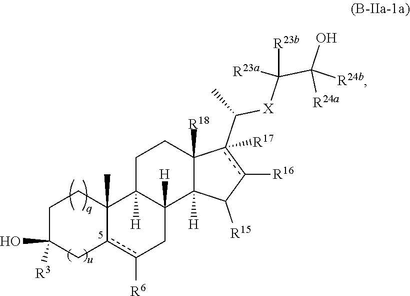

or a pharmaceutically acceptable salt thereof.

In some embodiments, the compound of formula (B-IIa-1a) is a compound of formula (B-IIa-1a-i):

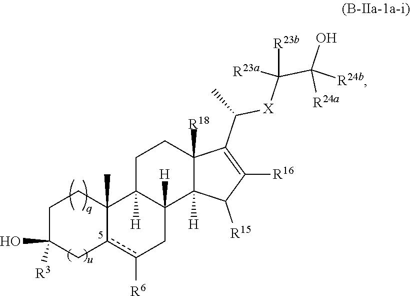

or a pharmaceutically acceptable salt thereof.

In some embodiments, the compound of formula (B-IIa-1a-i) is a compound of formula (B-IIa-1a-ia):

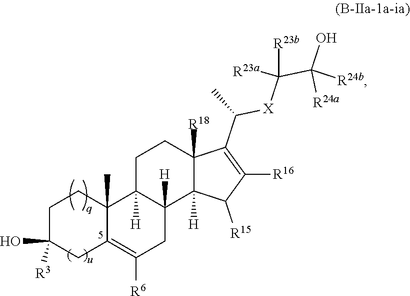

or a pharmaceutically acceptable salt thereof.

In some embodiments, the compound of formula (B-IIa-1a-i) is a compound of formula (B-IIa-1a-ib):

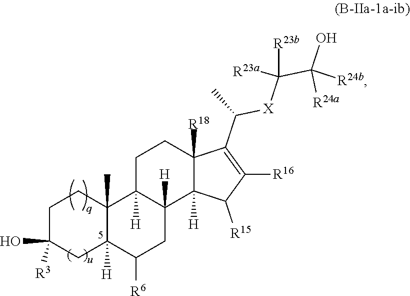

or a pharmaceutically acceptable salt thereof.

In some embodiments, the compound of formula (B-IIa-1a) is a compound of formula (B-IIa-1a-ii):

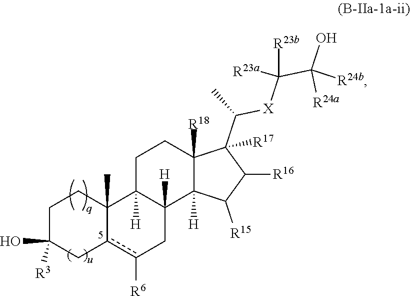

or a pharmaceutically acceptable salt thereof.

In some embodiments, the compound of formula (B-IIa-1a-ii) is a compound of formula (B-IIa-1a-iia):

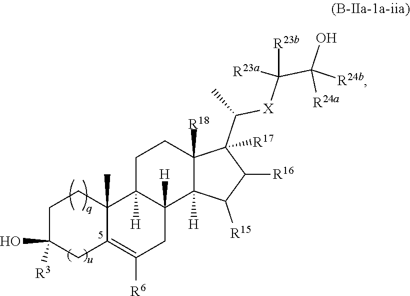

or a pharmaceutically acceptable salt thereof.

In some embodiments, the compound of formula (B-IIa-1a-ii) is a compound of formula (B-IIa-1a-iib):

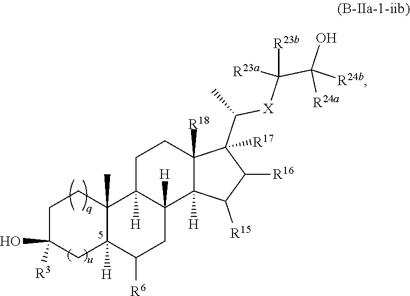

or a pharmaceutically acceptable salt thereof.

In some embodiments, the compound of formula (B-IIa-1) is a compound of formula (B-IIa-1b):

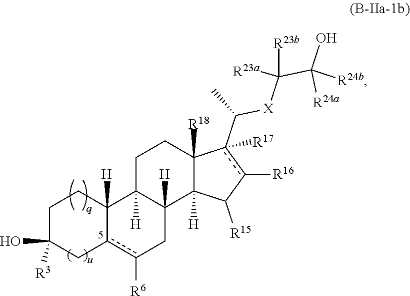

or a pharmaceutically acceptable salt thereof.

In some embodiments, the compound of formula (B-IIa-1b) is a compound of formula (B-IIa-1b-i):

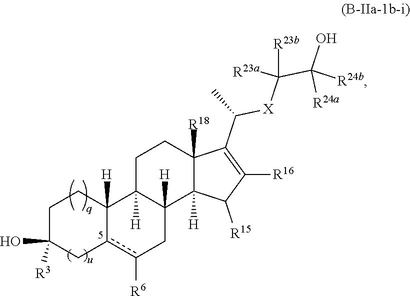

or a pharmaceutically acceptable salt thereof.

In some embodiments, the compound of formula (B-IIa-1b-i) is a compound of formula (B-IIa-1b-ia):

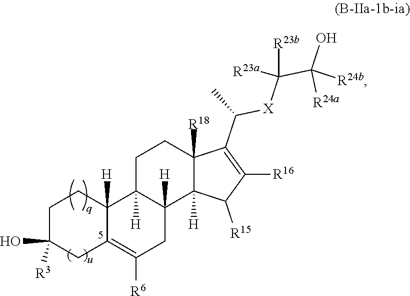

or a pharmaceutically acceptable salt thereof.

In some embodiments, the compound of formula (B-IIa-1b-i) is a compound of formula (B-IIa-1b-ib):

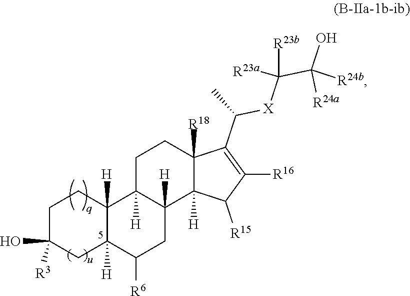

or a pharmaceutically acceptable salt thereof.

In some embodiments, the compound of formula (B-IIa-1b) is a compound of formula (B-IIa-1b-ii):

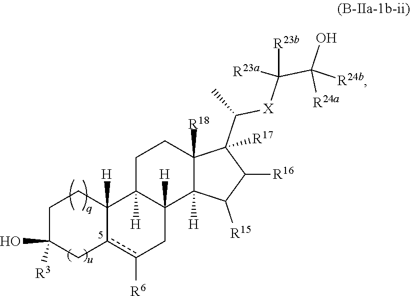

or a pharmaceutically acceptable salt thereof.

In some embodiments, the compound of formula (B-IIa-1b-ii) is a compound of formula (B-IIa-1b-iia):

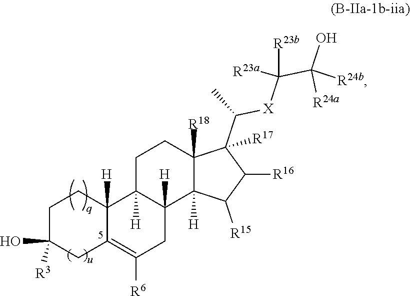

or a pharmaceutically acceptable salt thereof.

In some embodiments, the compound of formula (B-IIa-1a-ii) is a compound of formula (B-IIa-1a-iib):

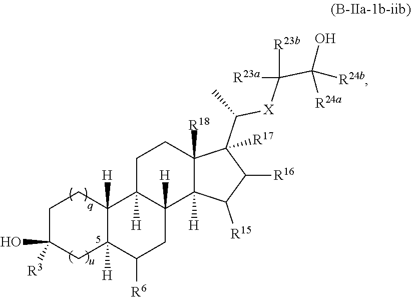

or a pharmaceutically acceptable salt thereof.

In some embodiments, the compound of formula (B-I) is a compound of formula (B-III):

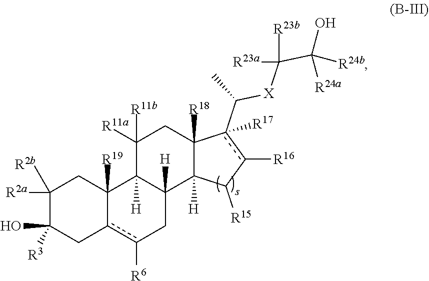

or a pharmaceutically acceptable salt thereof.

In some embodiments, the compound of formula (B-III) is a compound of formula (B-IIIa):

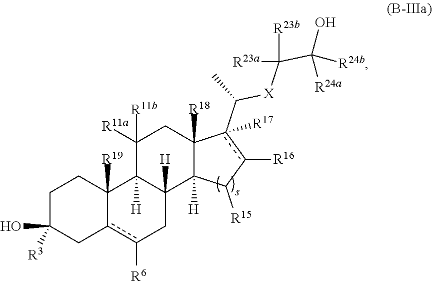

or a pharmaceutically acceptable salt thereof.

In some embodiments, the compound of formula (B-IIIa) is a compound of formula (B-IIIa-1):

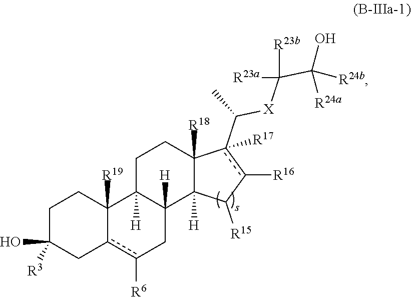

or a pharmaceutically acceptable salt thereof.

In some embodiments, the compound of formula (B-IIIa-1) is a compound of formula (B-IIIa-1a):

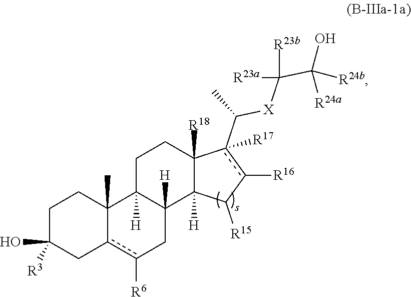

or a pharmaceutically acceptable salt thereof.

In some embodiments, the compound of formula (B-IIIa-1a) is a compound of formula (B-IIIa-1a-i):

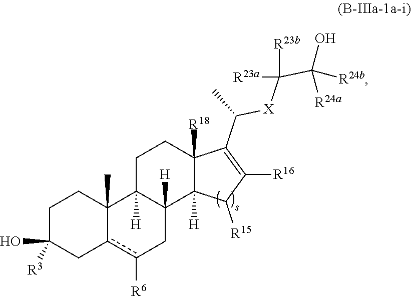

or a pharmaceutically acceptable salt thereof.

In some embodiments, the compound of formula (B-IIIa-1a-i) is a compound of formula (B-IIIa-1a-ia):

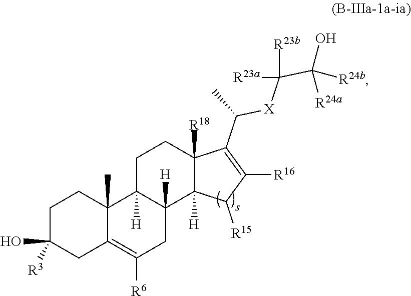

or a pharmaceutically acceptable salt thereof.

In some embodiments, the compound of formula (B-IIIa-1a-i) is a compound of formula (B-IIIa-1a-ib):

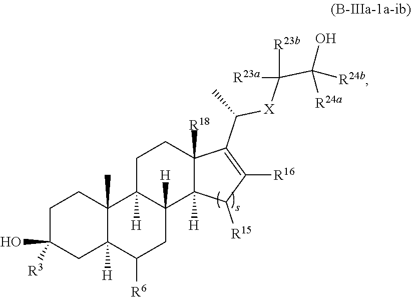

or a pharmaceutically acceptable salt thereof.

In some embodiments, the compound of formula (B-IIIa-1a) is a compound of formula (B-IIIa-1a-ii):

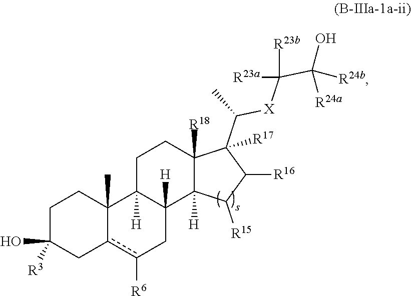

or a pharmaceutically acceptable salt thereof.

In some embodiments, the compound of formula (B-IIIa-1a-ii) is a compound of formula (B-IIIa-1a-iia):

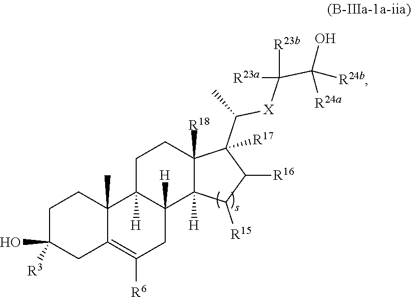

or a pharmaceutically acceptable salt thereof.

In some embodiments, the compound of formula (B-IIIa-1a-ii) is a compound of formula (B-IIIa-1a-ib):

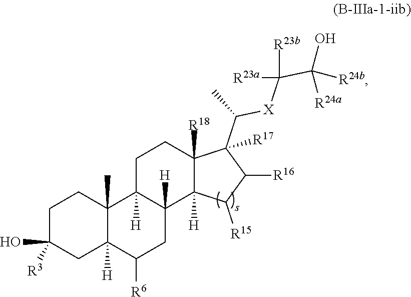

or a pharmaceutically acceptable salt thereof.

In some embodiments, the compound of formula (B-IIIa-1) is a compound of formula (B-IIIa-1b):

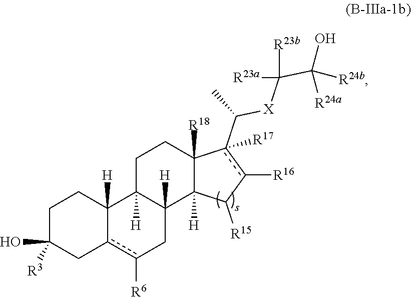

or a pharmaceutically acceptable salt thereof.

In some embodiments, the compound of formula (B-IIIa-1b) is a compound of formula (B-IIIa-1b-i):

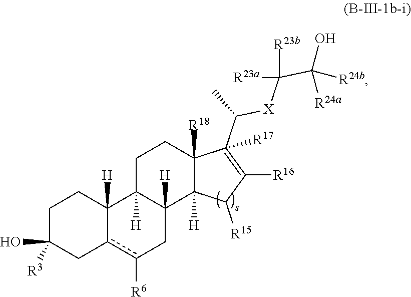

or a pharmaceutically acceptable salt thereof.

In some embodiments, the compound of formula (B-IIIa-1b-i) is a compound of formula (B-IIIa-1b-ia):

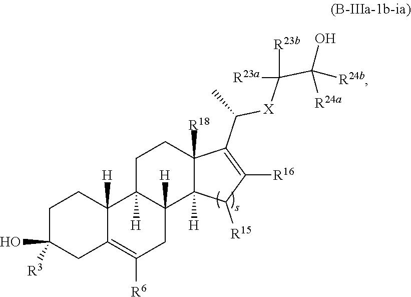

or a pharmaceutically acceptable salt thereof.

In some embodiments, the compound of formula (B-IIIa-1b-i) is a compound of formula (B-IIIa-1b-ib):

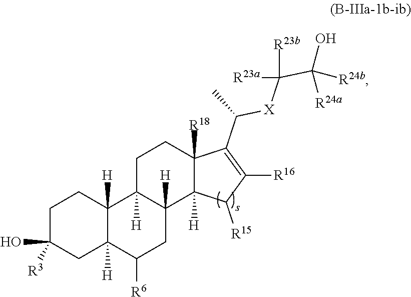

or a pharmaceutically acceptable salt thereof.

In some embodiments, the compound of formula (B-IIIa-1b) is a compound of formula (B-IIIa-1b-ii):

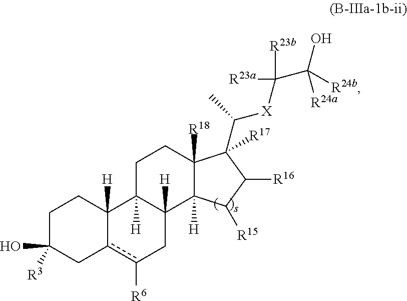

or a pharmaceutically acceptable salt thereof.

In some embodiments, the compound of formula (B-IIIa-1b-ii) is a compound of formula (B-IIIa-1b-iia):

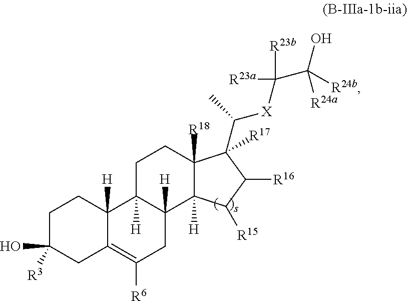

or a pharmaceutically acceptable salt thereof.

In some embodiments, the compound of formula (B-IIIa-1b-ii) is a compound of formula (B-IIIa-1b-iib):

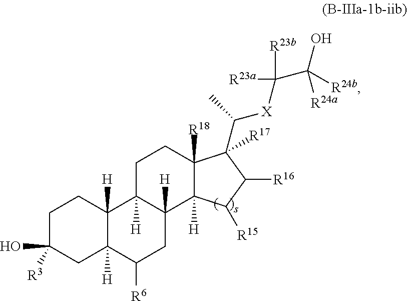

or a pharmaceutically acceptable salt thereof.

In some embodiments, the compound of formula (B-I) is a compound of formula (B-IV):

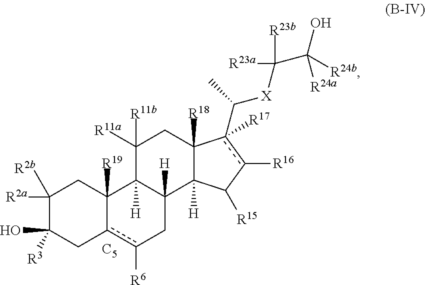

or a pharmaceutically acceptable salt thereof.

In some embodiments, the compound of formula (B-IV) is a compound of formula (B-IV-1):

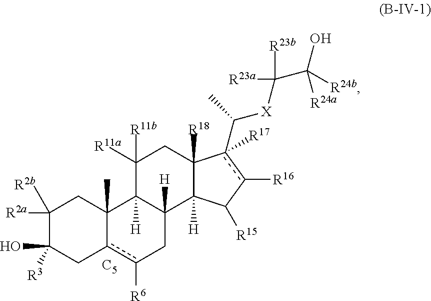

or a pharmaceutically acceptable salt thereof.

In some embodiments, the compound of formula (B-IV-1) is a compound of formula (B-IV-1a):

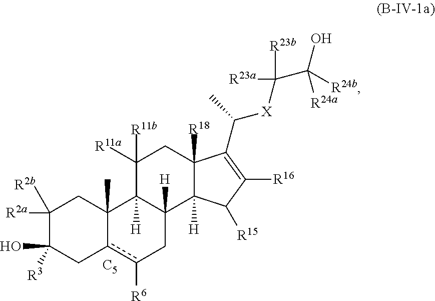

or a pharmaceutically acceptable salt thereof.

In some embodiments, the compound of formula (B-IV-1a) is a compound of formula (B-IV-1a-i):

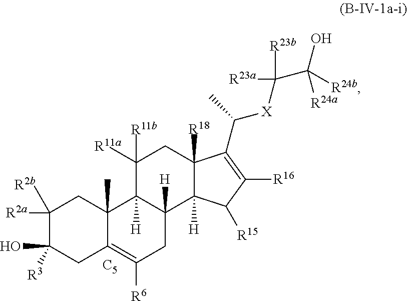

or a pharmaceutically acceptable salt thereof.

The compound or pharmaceutically acceptable salt thereof according to claim 40, wherein the compound of formula (B-IV-1a) is a compound of formula (B-IV-1a-ii):

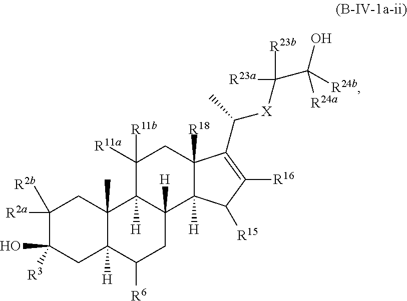

or a pharmaceutically acceptable salt thereof.

The compound or pharmaceutically acceptable salt thereof according to claim 38, wherein the compound of formula (B-IV) is a compound of formula (B-IV-2):

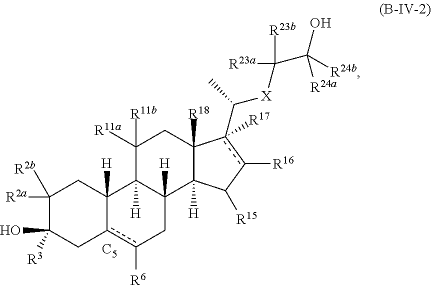

or a pharmaceutically acceptable salt thereof.

The compound or pharmaceutically acceptable salt thereof according to claim 43, wherein the compound of formula (B-IV-2) is a compound of formula (B-IV-2a):

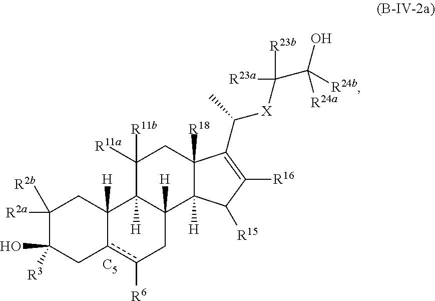

or a pharmaceutically acceptable salt thereof.

In some embodiments, the compound of formula (B-IV-2a) is a compound of formula (B-IV-2a-i):

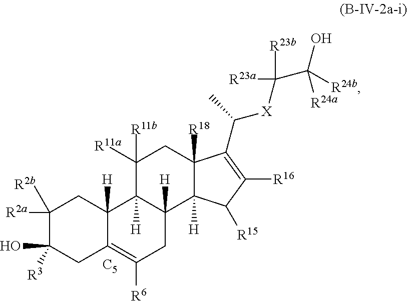

or a pharmaceutically acceptable salt thereof.

In some embodiments, the compound of formula (B-IV-2a) is a compound of formula (B-IV-2a-ii):

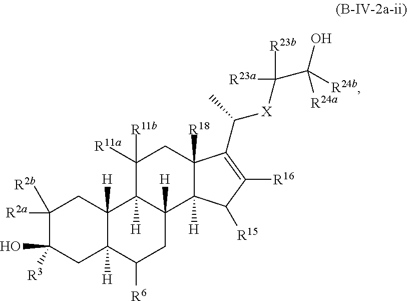

or a pharmaceutically acceptable salt thereof.

In some embodiments, the compound of formula (B-I) is a compound of formula (B-V):

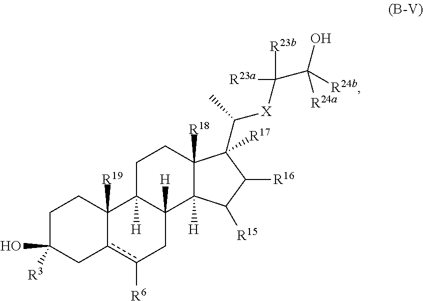

or a pharmaceutically acceptable salt thereof.

In some embodiments, the compound of formula (B-V) is a compound of formula (B-Va):

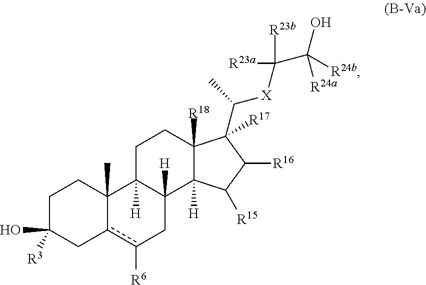

or a pharmaceutically acceptable salt thereof.

In some embodiments, the compound of formula (B-Va) is a compound of formula (B-Va-1):

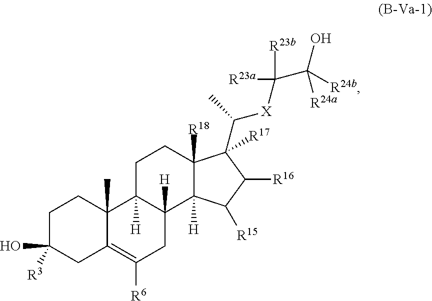

or a pharmaceutically acceptable salt thereof.

In some embodiments, the compound of formula (B-Va) is a compound of formula (B-Va-2):

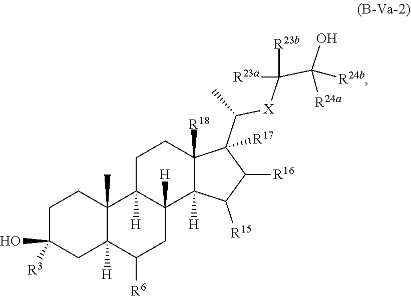

or a pharmaceutically acceptable salt thereof.

In some embodiments, the compound of formula (B-V) is a compound of formula (B-Vb):

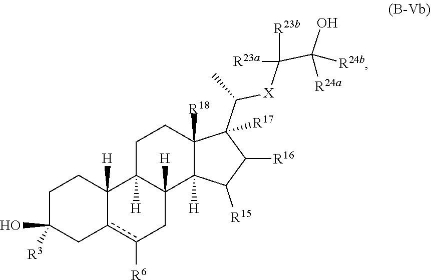

or a pharmaceutically acceptable salt thereof.

In some embodiments, the compound of formula (B-Vb) is a compound of formula (B-Vb-1):

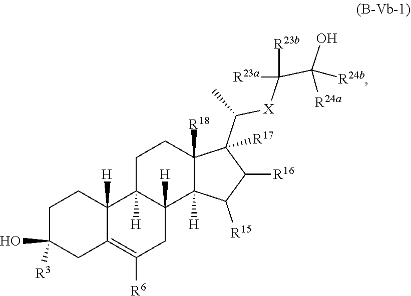

or a pharmaceutically acceptable salt thereof.

In some embodiments, the compound of formula (B-Vb) is a compound of formula (B-Vb-2):

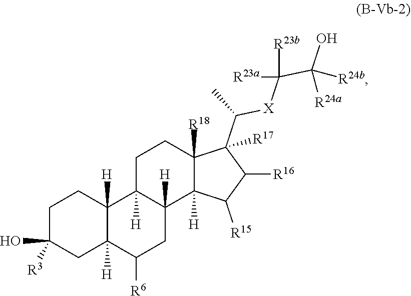

or a pharmaceutically acceptable salt thereof.
   Groups R^{2a} and R^{2b}—as they Relate to Formulae (B-I)-(B-Vb-2)

In certain embodiments, R^{2a} and R^{2b} is each independently hydrogen, halogen, substituted or unsubstituted C₁₋₆alkyl, substituted or unsubstituted cyclopropyl, or —OR^{A2}, wherein R^{A2} is hydrogen or substituted or unsubstituted C₁₋₆alkyl. In certain embodiments, each of R^{2a} and R^{2b} is hydrogen. In certain embodiments, each of R^{2a} and R^{2b} is independently halogen, e.g., fluoro, chloro, bromo, or iodo. In certain embodiments, each of R^{2a} and R^{2b} is independently fluoro or chloro. In certain embodiments, each of R^{2a} and R^{2b} is independently substituted or unsubstituted C₁₋₆alkyl, e.g., substituted or unsubstituted C₁₋₂ alkyl, substituted or unsubstituted C₂₋₃ alkyl, substituted or unsubstituted C₃₋₄ alkyl, substituted or unsubstituted C₄₋₅ alkyl, or substituted or unsubstituted C₅₋₆ alkyl. In certain embodiments, each of R^{2a} and R^{2b} is independently —CH₃, —CH₂CH₃, —CH₂CH₂CH₃, or cyclopropyl. In certain embodiments, each of R^{2a} and R^{2b} is independently —OR^{A2}. In certain embodiments, R^{A2} is hydrogen. In certain embodiments, R^{A2} is substituted or unsubstituted alkyl, e.g., substituted or unsubstituted C₁₋₆alkyl, substituted or unsubstituted C₁₋₂ alkyl, substituted or unsubstituted C₂₋₃ alkyl, substituted or unsubstituted C₃₋₄ alkyl, substituted or unsubstituted C₄₋₅alkyl, or substituted or unsubstituted C₅₋₆ alkyl. In certain embodiments, R^{A2} is hydrogen, —CH₃, —CH₂CH₃, or —CH₂CH₂CH₃, i.e., to provide a group of R^{2a} or R^{2b} each independently of formula-OH, —OCH₃, —OCH₂CH₃, or —OCH₂CH₂CH₃. In certain embodiments, one of R^{2a} and R^{2b} is a non-hydrogen substituent in the alpha configuration. In certain embodiments, one of R^{2a} and R^{2b} is i a non-hydrogen substituent in the beta configuration.

In some embodiments, each of R^{2a} and R^{2b} is hydrogen.

### Group R³—as it Relates to Formulae (B-I)-(B-Vb-2)

In certain embodiments, R³ is substituted or unsubstituted aliphatic, i.e., substituted or unsubstituted alkyl, substituted or unsubstituted alkenyl, substituted or unsubstituted alkynyl, or substituted or unsubstituted carbocyclyl.

In certain embodiments, R³ is substituted or unsubstituted C₁₋₆alkyl, substituted or unsubstituted C₂₋₆alkenyl, substituted or unsubstituted C₂₋₆alkynyl, or substituted or unsubstituted C₃₋₁₀carbocyclyl.

In certain embodiments, R³ is substituted or unsubstituted alkyl, e.g., substituted or unsubstituted C₁₋₆alkyl, substituted or unsubstituted C₁₋₂ alkyl, substituted or unsubstituted C₂₋₃ alkyl, substituted or unsubstituted C₃₋₄ alkyl, substituted or unsubstituted C₄₋₅ alkyl, or substituted or unsubstituted C₅₋₆ alkyl. Exemplary R³ C₁₋₆alkyl groups include, but are not limited to, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), n-hexyl (C₆), C₁₋₆ alkyl substituted with 1, 2, 3, 4, 5, 6, 7, 8, 9, 10, or more fluoro groups (e.g., —CF₃, —CH₂F, —CHF₂, difluoroethyl, and 2,2,2-trifluoro-1,1-dimethyl-ethyl), C₁₋₆alkyl substituted with 1, 2, 3, 4, 5, 6, 7, 8, 9, 10, or more chloro groups (e.g., —CH₂Cl, —CHCl₂), and C₁₋₆alkyl substituted with alkoxy groups (e.g., —CH₂OCH₃, —CH₂OCH₂CH₃, —CH₂O-cyclopropyl). In certain embodiments, R³ is substituted alkyl, e.g., R³ is haloalkyl, alkoxyalkyl, or aminoalkyl. In certain embodiments, R³ is Me, Et, n-Pr, n-Bu, i-Bu, fluoromethyl, chloromethyl, difluoromethyl, trifluoromethyl, trifluoroethyl, difluoroethyl, 2,2,2-trifluoro-1,1-dimethyl-ethyl, methoxymethyl, methoxyethyl, or ethoxymethyl.

In certain embodiments, R³ is unsubstituted C₁₋₆alkyl. In some embodiments, R³ is selected from the group consisting of CH₃, —CH₂CH₃, or —CH₂CH₂CH₃. In some embodiments, R³ is —CH₂CH₃.

In certain embodiments, R³ is alkyl substituted with one or more fluorine atoms; e.g., R³ is —CH₂F, —CHF₂, or —CF₃.

In certain embodiments, R³ is alkyl substituted with one or more —OR^{A3} groups, wherein R^{A3} is hydrogen or substituted or unsubstituted alkyl. In certain embodiments, R³ is CH₂OR^{A3}, e.g., wherein R^{A3} is hydrogen, —CH₃, —CH₂CH₃, or —CH₂CH₂CH₃.

In certain embodiments, R³ is substituted or unsubstituted alkenyl, e.g., substituted or unsubstituted C₂₋₆alkenyl, substituted or unsubstituted C₂₋₃alkenyl, substituted or unsubstituted C₃₋₄ alkenyl, substituted or unsubstituted C₄₋₅ alkenyl, or substituted or unsubstituted C₅₋₆ alkenyl. In certain embodiments, R³ is ethenyl (C₂), propenyl (C₃), or butenyl (C₄), unsubstituted or substituted with one or more substituents selected from the group consisting of alkyl, halo, haloalkyl, alkoxyalkyl, or hydroxyl. In certain embodiments, R³ is ethenyl, propenyl, or butenyl, unsubstituted or substituted with alkyl, halo, haloalkyl, alkoxyalkyl, or hydroxy. In certain embodiments, R³ is ethenyl.

In certain embodiments, R³ is substituted or unsubstituted alkynyl, e.g., substituted or unsubstituted C₂₋₆alkynyl, substituted or unsubstituted C₂₋₃ alkynyl, substituted or unsubstituted C₃₋₄ alkynyl, substituted or unsubstituted C₄₋₅ alkynyl, or substituted or unsubstituted C₅₋₆ alkynyl. Exemplary substituted or unsubstituted R³ alkynyl groups include, but are not limited to, ethynyl, propynyl, or butynyl, unsubstituted or substituted with alkyl, halo, haloalkyl (e.g., CF₃), alkoxyalkyl, cycloalkyl (e.g., cyclopropyl or cyclobutyl), or hydroxyl. In certain embodiments, R³ is selected from the group consisting of trifluoroethynyl, cyclopropylethynyl, cyclobutylethynyl, and propynyl, fluoropropynyl, and chloroethynyl. In certain embodiments, R³ is ethynyl (C₂), propynyl (C₃), or butynyl (C₄), unsubstituted or substituted with one or more substituents selected from the group consisting of substituted or unsubstituted aryl, substituted or unsubstituted heteroaryl, substituted or unsubstituted carbocyclyl, and substituted or unsubstituted heterocyclyl. In certain embodiments, R³ is ethynyl (C₂), propynyl (C₃), or butynyl (C₄) substituted with substituted phenyl. In certain embodiments, the phenyl substituent is further substituted with one or more substituents selected from the group consisting of halo, alkyl, trifluoroalkyl, alkoxy, acyl, amino or amido. In certain embodiments, R³ is ethynyl (C₂), propynyl (C₃), or butynyl (C₄) substituted with substituted or unsubstituted pyrrolyl, imidazolyl, pyrazolyl, oxazoyl, thiazolyl, isoxazoyl, 1,2,3-triazolyl, 1,2,4-triazolyl, oxadiazolyl, thiadiazolyl, or tetrazolyl.

In certain embodiments, R³ is ethynyl, propynyl, or butynyl, unsubstituted or substituted with alkyl, halo, haloalkyl, alkoxyalkyl, or hydroxyl. In certain embodiments, R³ is ethynyl or propynyl, substituted with substituted or unsubstituted aryl. In certain embodiments, R³ is ethynyl or propynyl, substituted with phenyl unsubstituted or substituted with halo, alkyl, alkoxy, haloalkyl, trihaloalkyl, or acyl. In certain embodiments, R³ is ethynyl or propynyl, substituted with substituted or unsubstituted carbocyclyl. In certain embodiments, R³ is ethynyl or propynyl, substituted with substituted or unsubstituted cyclopropyl, cyclobutyl, cyclopentyl, or cyclohexyl. In certain embodiments, R³ is ethynyl or propynyl, substituted with substituted or unsubstituted heteroaryl. In certain embodiments, R³ is ethynyl or propynyl, substituted with substituted or unsubstituted pyridinyl, or pyrimidinyl. In certain embodiments, R³ is ethynyl or propynyl, substituted with substituted or unsubstituted pyrrolyl, imidazolyl, pyrazolyl, oxazoyl, thiazolyl, isoxazoyl, 1,2,3-triazolyl, 1,2,4-triazolyl, oxadiazolyl, thiadiazolyl, tetrazolyl. In certain embodiments, R³ is ethynyl or propynyl, substituted with substituted or unsubstituted heterocyclyl. In certain embodiments, R³ is ethynyl or propynyl, substituted with substituted or unsubstituted pyrrolidinyl, piperidinyl, piperazinyl, or morpholinyl. In certain embodiments, R³ is propynyl or butynyl, substituted with hydroxyl or alkoxy. In certain embodiments, R³ is propynyl or butynyl, substituted with methoxy or ethoxy. In certain embodiments, R³ is ethynyl or propynyl, substituted with chloro. In certain embodiments, R³ is ethynyl or propynyl, substituted with trifluoromethyl.

In certain embodiments, R³ is substituted or unsubstituted carbocyclyl, e.g., substituted or unsubstituted C₃₋₁₀carbocyclyl, substituted or unsubstituted C₃₋₆carbocyclyl, substituted or unsubstituted C₃₋₄ carbocyclyl, substituted or unsubstituted C₄₋₅ carbocyclyl, or substituted or unsubstituted C₅₋₆ carbocyclyl. In certain embodiments, R³ is substituted or unsubstituted cyclopropyl or substituted or unsubstituted cyclobutyl.

Groups R⁹, R^{11a}, and R^{11b}—as they Relate to Formulae (B-I)-(B-Vb-2)

In certain embodiments, R⁹ is absent or hydrogen. In some embodiments, R⁹ is hydrogen. In some embodiments, the bond between C9 and C11 is a double bond and R⁹ is absent. In some embodiments, the bond between C9 and C11 is a double bond, R⁹ is absent, R^{11a} is selected from the group consisting of hydrogen, halogen, substituted or unsubstituted C₁₋₆alkyl, and —OR^{A11}, wherein R^{A11} is hydrogen or substituted or unsubstituted C₁₋₆alkyl, and R^{11b} is absent. In some embodiments, the bond between C9 and C11 is a single bond, R⁹ is hydrogen, and R¹¹ª is selected from the group consisting of hydrogen, halogen or substituted or unsubstituted C₁₋₆alkyl or —OR^{A11}, wherein R^{A11} is hydrogen or substituted or unsubstituted C₁₋₆alkyl, and R^{11b} is hydrogen. In some embodiments, the bond between C9 and C11 is a single bond, R⁹ is hydrogen, R^{11a} is hydrogen or —OR^{A11}, wherein R^{A11} is hydrogen or substituted or unsubstituted C₁₋₆alkyl, and R^{11b} is hydrogen.

In certain embodiments, R^{11a} is hydrogen or —OR^{A11}, wherein R^{A11} is hydrogen or substituted or unsubstituted C₁₋₆alkyl, and R^{11b} is hydrogen; or R^{11a} and R^{11b} are joined to form an oxo (═O) group.

In certain embodiments, R^{11a} and R^{11b} are joined to form an oxo (═O) group.

In certain embodiments, R^{11a} is —OR^{A11} and R^{11b} is hydrogen. In certain embodiments, wherein R^{11a} is —OR^{A11}, R^{11a} is in the alpha or beta configuration. In certain embodiments, wherein R¹¹ª is —OR^{A11}, R^{11a} is in the alpha configuration. In certain embodiments, wherein R^{11a} is —OR^{A11}, R^{11a} is in the beta configuration. In certain embodiments, R^{A11} is hydrogen. In certain embodiments, R^{A11} is substituted or unsubstituted alkyl, e.g., substituted or unsubstituted C₁₋₆alkyl, substituted or unsubstituted C₁₋₂ alkyl, substituted or unsubstituted C₂₋₃ alkyl, substituted or unsubstituted C₃₋₄ alkyl, substituted or unsubstituted C₄₋₅alkyl, or substituted or unsubstituted C₅₋₆ alkyl. In certain embodiments, R^{A11} is hydrogen, —CH₃, —CH₂CH₃, or —CH₂CH₂CH₃, i.e., to provide a group R^{11a} of formula —OH, —OCH₃, —OCH₂CH₃, or —OCH₂CH₂CH₃.

In some embodiments, R^{11b} is absent or hydrogen. In some embodiments, R^{11b} is hydrogen. In some embodiments, the bond between C9 and C11 is a double bond, and R^{11b} is absent. In some embodiments, the bond between C9 and C11 is a single bond, and R^{11b} is hydrogen.

In some embodiments, each of R^{11a} and R^{11b} is hydrogen.

In some embodiments, R⁹ and R^{11a} are taken together with the carbon atoms to which they are attached to form a substituted or unsubstituted C₃₋₆carbocyclyl.

Groups R⁵ and R⁶—as they Relate to Formulae (B-I)-(B-Vb-2)

In some embodiments R⁵ is in the alpha or beta configuration. In certain embodiments R⁵ is in the alpha configuration. In certain embodiments R⁵ is in the beta configuration. In some embodiments, the bond between C5 and C6 is a double bond and R⁵ is absent. In some embodiments, the bond between C5 and C6 is a single bond and R⁵ is hydrogen, halogen, or substituted or unsubstituted C₁₋₆alkyl. In some embodiments, the bond between C5 and C6 is a single bond and R⁵ is hydrogen, halogen, or substituted or unsubstituted C₁₋₆alkyl in the beta configuration. In some embodiments, the bond between C5 and C6 is a single bond and R⁵ is hydrogen, halogen, or substituted or unsubstituted C₁₋₆ alkyl in the alpha configuration. In some embodiments, the bond between C5 and C6 is a single bond and R⁵ is hydrogen in the alpha configuration.

In certain embodiments, R⁵ is absent, hydrogen, halogen, or substituted or unsubstituted C₁₋₆alkyl. In some embodiments, R⁵ is halogen. In some embodiments, R⁵ is substituted C₁₋₆alkyl. In certain embodiments, R⁵ is substituted or unsubstituted alkyl, e.g., substituted or unsubstituted C₁₋₆alkyl, substituted or unsubstituted C₁₋₂ alkyl, substituted or unsubstituted C₂₋₃ alkyl, substituted or unsubstituted C₃₋₄ alkyl, substituted or unsubstituted C₄₋₅alkyl, or substituted or unsubstituted C₅₋₆ alkyl. In some embodiments, R⁵ is substituted or unsubstituted C₁₋₆alkyl. In some embodiments, R⁵ is unsubstituted C₁₋₆alkyl. In some embodiments, R⁵ is unsubstituted C₁ alkyl. In some embodiments, R⁵ is —CH₃.

In some embodiments, R⁶ is hydrogen, substituted or unsubstituted alkyl, or halogen. In certain embodiments, R⁶ is hydrogen. In certain embodiments, R⁶ is halogen, e.g., fluoro. In certain embodiments, R⁶ is substituted or unsubstituted alkyl, e.g., substituted or unsubstituted C₁₋₆alkyl, substituted or unsubstituted C₁₋₂ alkyl, substituted or unsubstituted C₂₋₃ alkyl, substituted or unsubstituted C₃₋₄ alkyl, substituted or unsubstituted C₄₋₅ alkyl, or substituted or unsubstituted C₅₋₆ alkyl. In certain embodiments, R⁶ is C₁ alkyl, e.g., —CH₃ or —CF₃. In certain embodiments, R⁶ is hydrogen, —CH₃, or —F. In certain embodiments, the bond between C5 and C6 is a single bond and R⁶ is a non-hydrogen substituent in the alpha configuration. In certain embodiments, the bond between C5 and C6 is a single bond and R⁶ is a non-hydrogen substituent in the beta configuration.

Groups R¹⁵, R¹⁶, and R¹⁷—as they Relate to Formulae (B-I)-(B-Vb-2)

In certain embodiments, R¹⁵ is hydrogen, halogen, —OH, —NH₂, substituted or unsubstituted C₁₋₆alkyl (unsub. methyl), substituted or unsubstituted C₂₋₆alkenyl, substituted or unsubstituted C₂₋₆alkynyl, substituted or unsubstituted C₁₋₆alkoxy, substituted or unsubstituted C₃₋₆carbocyclyl (unsub. cyclopropyl) or substituted or unsubstituted 3- to 10-membered heterocyclyl. In some embodiments, R¹⁵ is hydrogen, substituted or unsubstituted C₁₋₆alkyl or substituted or unsubstituted C₃₋₆carbocyclyl. In certain embodiments, R¹⁵ is hydrogen. In some embodiments, R¹⁵ is substituted or unsubstituted C₁₋₆alkyl. In some embodiments, R¹⁵ is unsubstituted C₁₋₆alkyl. In some embodiments, R¹⁵ is —CH₃. In some embodiments, R¹⁵ is substituted or unsubstituted C₃₋₆carbocyclyl. In some embodiments, R¹⁵ is substituted with substituted or unsubstituted cyclopropyl, cyclobutyl, cyclopentyl, or cyclohexyl. In some embodiments R¹⁵ is unsubstituted cyclopropyl. In some embodiments, R¹⁵ is hydrogen, —CH₃ or cyclopropyl.

In certain embodiments, R¹⁶ is hydrogen, halogen, —OH, —NH₂, substituted or unsubstituted C₁₋₆alkyl (unsub. methyl), substituted or unsubstituted C₂₋₆alkenyl, substituted or unsubstituted C₂₋₆alkynyl, substituted or unsubstituted C₁₋₆alkoxy, substituted or unsubstituted C₃₋₆carbocyclyl (unsub. cyclopropyl) or substituted or unsubstituted 3- to 10-membered heterocyclyl. In some embodiments, R¹⁶ is hydrogen, or unsubstituted C₁₋₆alkyl. In some embodiments, R¹⁶ is hydrogen, or —CH₃. In some embodiments, R¹⁶ is hydrogen. In certain embodiments, R¹⁶ is substituted or unsubstituted C₁₋₆alkyl. In some embodiments, R¹⁶ is unsubstituted C₁₋₆alkyl. In some embodiments, R¹⁶ is —CH₃.

In certain embodiments, R¹⁵ and R¹⁶ are taken together with the carbon atoms to which they are attached to form a substituted or unsubstituted C₃-6 carbocyclyl. In some embodiments, R¹⁵ and R¹⁶ are taken together with the carbon atoms to which they are attached to form a substituted or unsubstituted cyclopropyl, cyclobutyl, cyclopentyl or cyclohexyl. In some embodiments, R¹⁵ and R¹⁶ are taken together with the carbon atoms to which they are attached to form an unsubstituted cyclopropyl, cyclobutyl, cyclopentyl or cyclohexyl. In some embodiments, R¹⁵ and R¹⁶ are taken together with the carbon atoms to which they are attached to form an unsubstituted cyclopropyl.

In certain embodiments, R¹⁷ is hydrogen or substituted or unsubstituted C₁₋₆alkyl. In some embodiments, R¹⁷ is absent, and the bond between C16 and C17 is a double bond. In some embodiments, R¹⁷ is substituted or unsubstituted C₁₋₆alkyl. In some embodiments, R¹⁷ is hydrogen or unsubstituted C₁₋₆alkyl. In some embodiments, R¹⁷ is hydrogen or —CH₃. In some embodiments, R¹⁷ is hydrogen. In some embodiments, R¹⁷ is an unsubstituted C₁alkyl. In some embodiments R¹⁷ is —CH₃.

In certain embodiments, R¹⁶ and R¹⁷, taken together with the carbon atoms to which they are attached to form a substituted or unsubstituted C₃-6 carbocyclyl. In some embodiments, R¹⁶ and R¹⁷ are taken together with the carbon atoms to which they are attached to form a substituted or unsubstituted cyclopropyl, cyclobutyl, cyclopentyl or cyclohexyl. In some embodiments, R¹⁶ and R¹⁷ are taken together with the carbon atoms to which they are attached to form an unsubstituted cyclopropyl, cyclobutyl, cyclopentyl or cyclohexyl. In some embodiments, R¹⁶ and R¹⁷ are taken together with the carbon atoms to which they are attached to form an unsubstituted cyclopropyl.

Groups R¹⁸ and R¹⁹—as they Relate to Formulae (B-I)-(B-Vb-2)

In some embodiments R¹⁸ is hydrogen, substituted or unsubstituted C₁₋₆alkyl, or OR^{A18}, wherein each instance of R^{A18} is independently substituted or unsubstituted C₁₋₆alkyl. In some embodiments, R¹⁸ is hydrogen, unsubstituted C₂₋₆alkyl, C₁₋₆alkyl substituted with OR^{A18}, or —OR^{A18}, wherein each instance of R^{A18} is independently substituted or unsubstituted C₁₋₆alkyl. In some embodiments, R¹⁸ is hydrogen, unsubstituted C₁₋₆alkyl, C₁₋₆alkyl substituted with OR^{A18}, or —OR^{A18}, wherein each instance of R^{A18} is independently unsubstituted C₁₋₆alkyl. In some embodiments, R¹⁸ is unsubstituted C₁₋₆alkyl. In some embodiments, R¹⁸ is —CH₃, —CH₂CH₃, or —CH(CH₃)₂. In some embodiments, R¹⁸ is —CH₃. In some embodiments, R¹⁸ is —CH₂CH₃, or —CH(CH₃)₂. In some embodiments, R¹⁸ is substituted C₁₋₆alkyl. In some embodiments, R¹⁸ is C₁₋₆alkyl substituted with one or more OR^{A18}, wherein each instance of R^{A18} is independently substituted or unsubstituted C₁₋₆alkyl. In some embodiments, R¹⁸ is C₁₋₆alkyl substituted with OR^{A18}, wherein R^{A18} is substituted or unsubstituted C₁₋₆alkyl. In some embodiments, R¹⁸ is —OR^{A18}, wherein R^{A18} is substituted or unsubstituted C₁₋₆alkyl. In some embodiments, R¹⁸ is hydrogen, —CH₃, —CH₂OCH₃, —OCH₃, —CH₂CH₃, or —CH(CH₃)₂. In some embodiments, R¹⁸ is hydrogen, —CH₂OCH₃, —OCH₃, —CH₂CH₃, or —CH(CH₃)₂. In some embodiments, R¹⁸ is methyl. In some embodiments R¹⁸ is hydrogen.

In some embodiments R¹⁹ is hydrogen, substituted or unsubstituted C₁₋₆alkyl, or OR^{A19}; wherein each instance of R^{A19} is independently substituted or unsubstituted C₁₋₆alkyl. In some embodiments, R¹⁹ is hydrogen, unsubstituted C₁₋₆alkyl, C₁₋₆alkyl substituted with OR^{A19}, or —OR^{A19}; wherein each instance of R^{A19} is independently substituted or unsubstituted C₁₋₆alkyl. In some embodiments, R¹⁹ is hydrogen, unsubstituted C₁₋₆alkyl, C₁₋₆alkyl substituted with OR^{A19}, or —OR^{A19}; wherein each instance of R^{A19} is independently unsubstituted C₁₋₆alkyl. In some embodiments, R¹⁹ is unsubstituted C₁₋₆alkyl. In some embodiments, R¹⁹ is —CH₃, —CH₂CH₃, or —CH(CH₃)₂. In some embodiments, R¹⁹ is —CH₃. In some embodiments, R¹⁹ is —CH₂CH₃, or —CH(CH₃)₂. In some embodiments, R¹⁹ is substituted C₁₋₆alkyl. In some embodiments, R¹⁹ is C₁₋₆alkyl substituted with one or more OR^{A19}; wherein each instance of R^{A19} is independently substituted or unsubstituted C₁₋₆ alkyl. In some embodiments, R¹⁹ is C₁₋₆alkyl substituted with OR^{A19}, wherein R^{A19} is substituted or unsubstituted C₁₋₆alkyl. In some embodiments, R¹⁹ is —OR^{A19}; wherein R^{A19} is substituted or unsubstituted C₁₋₆alkyl. In some embodiments, R¹⁹ is hydrogen, —CH₃, —CH₂OCH₃, —OCH₃, —CH₂CH₃, or —CH(CH₃)₂. In some embodiments, R¹⁹ is hydrogen, —CH₂OCH₃, —OCH₃, —CH₂CH₃, or —CH(CH₃)₂. In some embodiments, R¹⁹ is hydrogen, —CH₃, or —CH₂CH₃. In some embodiments, R¹⁹ is —CH₃. In some embodiments, R¹⁹ is —CH₂CH₃. In some embodiments R¹⁹ is hydrogen or —CH₃. In some embodiments, R¹⁹ is hydrogen. In some embodiments R¹⁹ is —CH₃.

Groups X, n, R^{23a}, R^{23b}, R^{24a}, and R^{24b}—as they Relate to Formulae (B-I)-(B-Vb-2)

In certain embodiments X is —(C(R^{X})₂)ₙ— or —O—, wherein R^{X} is hydrogen or fluorine, or one R^{X} group and R^{23b} are joined to form a double bond.

In certain embodiments, X is —O—. In certain embodiments, X is —CH₂—. In certain embodiments, X is —CF₂—.

In certain embodiments, n is selected from 1, 2 or 3. In some embodiments, n is 1. In some embodiments, n is 2. In some embodiments, n is 3.

In some embodiments, X is —(C(R^{X})₂—, wherein R^{X} is hydrogen or fluorine. In some embodiments, X is —CH₂—.

In some embodiments, X is —(C(R^{X})₂)₂—, wherein R^{X} is hydrogen or fluorine. In some embodiments, X is —CH₂—.

In some embodiments, each of R^{23a} and R^{23b} is independently hydrogen or fluorine. In some embodiments, each of R^{23a} and R^{23b} is independently hydrogen. In some embodiments, each of R^{23a} and R^{23b} is independently fluorine. In certain embodiments, at least one of R^{23a} and R^{23b} is hydrogen. In certain embodiments, R^{X} and R^{23b} are joined to form a double bond, e.g., cis or trans double bond.

In some embodiments, R^{24a} is a non-hydrogen group selected from the group consisting of substituted or unsubstituted C₁₋₆alkyl, substituted or unsubstituted C₂₋₆alkenyl, substituted or unsubstituted C₂₋₆alkynyl, substituted or unsubstituted C₃₋₁₀carbocyclyl, substituted or unsubstituted 3- to 10-membered heterocyclyl, substituted or unsubstituted C₆₋₁₄aryl, and substituted or unsubstituted 5- to 10-membered heteroaryl; and R^{24b} is hydrogen or a substituted or unsubstituted C₁₋₆alkyl; or R^{24a} and R^{24b}, taken together with the carbon atom to which they are attached, form a substituted or unsubstituted C₃₋₆carbocyclyl, or substituted or unsubstituted 3- to 10-membered heterocyclyl.

In some embodiments, R^{24a} is selected from the group consisting of substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl.

In some embodiments, R^{24b} is hydrogen, substituted or unsubstituted C₁₋₆alkyl group, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl.

In some embodiments, R^{24a} is a substituted or unsubstituted C₁₋₆alkyl. In some embodiments, R^{24a} is unsubstituted C₁₋₆alkyl. In some embodiments, R^{24a} is substituted C₁₋₆alkyl. In some embodiments, R^{24a} is —CH₃, —CH₂CH₃, —CH(CH₃)₂, or —CF₃. In some embodiments R^{24a} is —CH₃, —CH(CH₃)₂ or CF₃. In some embodiments, R^{24a} is methyl. In some embodiments, R^{24a} is isopropyl. In some embodiments, R^{24a} is CF₃.

In some embodiments, R^{24b} is hydrogen or substituted or unsubstituted C₁₋₆alkyl. In some embodiments, R^{24b} is hydrogen or unsubstituted C₁₋₆alkyl. In some embodiments, R^{24b} is hydrogen. In some embodiments R^{24b} is unsubstituted C₁₋₆alkyl. In some embodiments, R^{24b} is —CH₃.

In some embodiments, R^{24a} and R^{24b}, taken together with the carbon atom to which they are attached, form a substituted or unsubstituted C₃₋₆carbocyclyl, or substituted or unsubstituted 3- to 10-membered heterocyclyl. In some embodiments, R^{24a} and R^{24b}, taken together with the carbon atom to which they are attached, form a substituted or unsubstituted C₃₋₆carbocyclyl. In some embodiments, R^{24a} and R^{24b} taken together with the carbon atom to which they are attached, form an unsubstituted C₃₋₆carbocyclyl. In some embodiments, R^{24a} and R^{24b} taken together with the carbon atom to which they are attached, form a cyclopropyl. In some embodiments, R^{24a} and R^{24b}, taken together with the carbon atom to which they are attached, form a substituted or unsubstituted 3- to 10-membered heterocyclyl.

q, r and s—as they Relate to Formulae (B-I)-(B-Vb-2)

In some embodiments, q is 0, 1 or 2. In some embodiments, q is 0. In some embodiments, q is 1. In some embodiments, q is 2.

In some embodiments, u is 1 or 2. In some embodiments, u is 1. In some embodiments, u is 2.

In some embodiments, s is 1 or 2. In some embodiments, s is 1. In some embodiments, s is 2.

In some embodiments, q is 0 and u is 1. In some embodiments, q is 0 and u is 2. In some embodiments q is 1 and u is 1. In some embodiments, q is 1 and u is 2. In some embodiments, q is 2 and u is 1. In some embodiments, q is 2 and u is 2. In some embodiments q is 0, u is 1, and s is 1. In some embodiments, q, u, and s are each independently 1.

In some embodiments, the compound of Formula (B-I) is any one of Compounds B-1 to B-24 in Table B-1 or pharmaceutically acceptable salts thereof. In some embodiments, the compound of Formula (B-I) is any one of Compounds B-1 to B-24 in Table B-1. In some embodiments, the compound of Formula (B-I) is a pharmaceutically acceptable salt of any one of Compounds B-1 to B-24. In some embodiments, the compound of Formula (B-I) is any one of Compounds B-1, B-3, B-4, B-10, B-11, B-12, B-14, B-15, B-18, B-20, B-23, or pharmaceutically acceptable salts thereof. In some embodiments, the compound of Formula (B-I) is any one of Compounds B-1, B-3, B-4, B-10, B-11, B-12, B-14, B-15, B-18, B-20, or B-23. In some embodiments, the compound of Formula (B-I) is a pharmaceutically acceptable salt of any one of Compounds B-1, B-3, B-4, B-10, B-11, B-12, B-14, B-15, B-18, B-20, or B-23. In some embodiments, the compound of Formula (B-I) is any one of Compounds B-1, B-2, B-3, B-4, B-5, B-6, B-13, B-14, B-22, B-23, B-24 or pharmaceutically acceptable salts thereof. In some embodiments, the compound of Formula (B-I) is any one of Compounds B-1, B-2, B-3, B-4, B-5, B-6, B-13, B-14, B-22, B-23, or B-24. In some embodiments, the compound of Formula (B-I) is a pharmaceutically acceptable salt of any one of Compounds B-1, B-2, B-3, B-4, B-5, B-6, B-13, B-14, B-22, B-23, or B-24.

TABLE B-1

| Cmpd | Structure |
|---|---|
| B-1 | 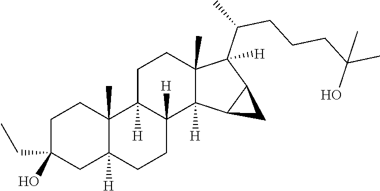 |
| B-2 | 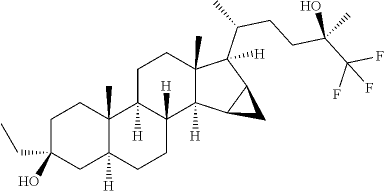 |
| B-3 | 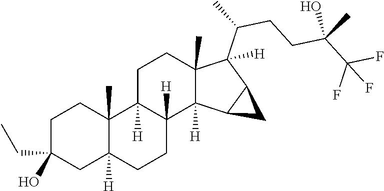 |
| B-4 | 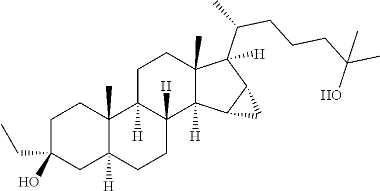 |
| B-5 | 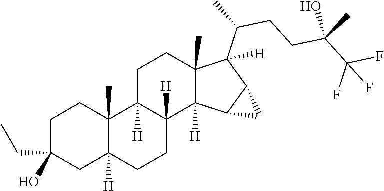 |
| B-6 | 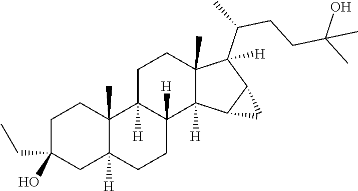 |
| B-7 | 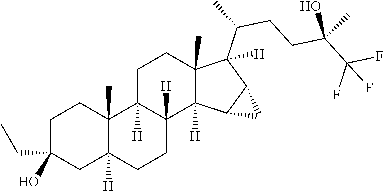 |
| B-8 | 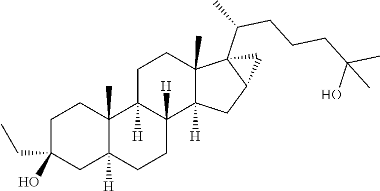 |
| B-10 | 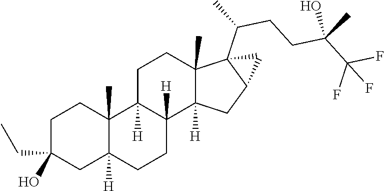 |
| B-11 | 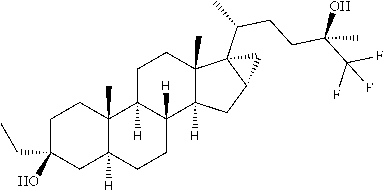 |
| B-12 | 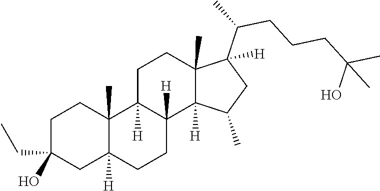 |
| B-13 | 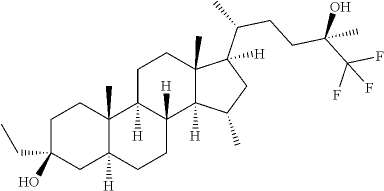 |
| B-14 | 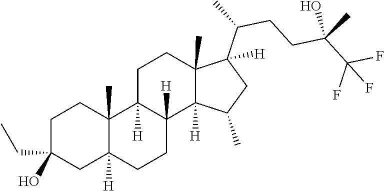 |
| B-15 | 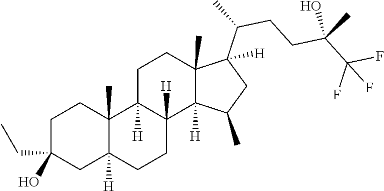 |
| B-16 | 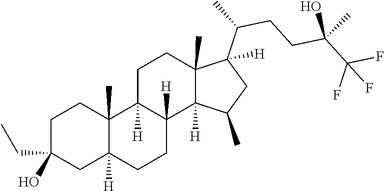 |
| B-17 | 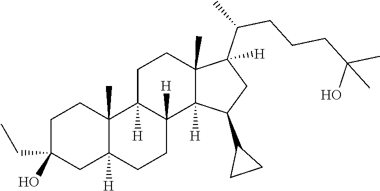 |
| B-18 | 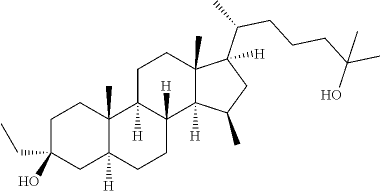 |
| B-19 | 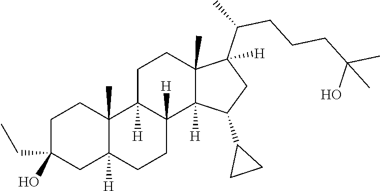 |
| B-20 | 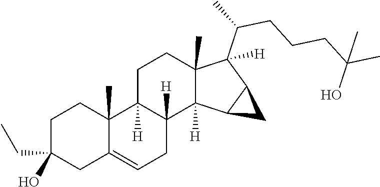 |
| B-21 | 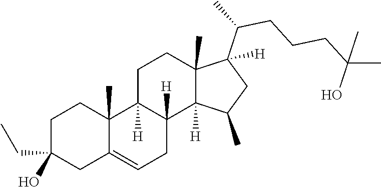 |
| B-22 | 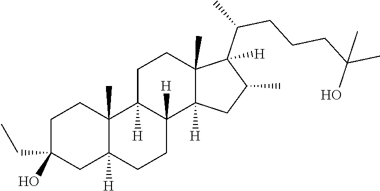 |
| B-23 | 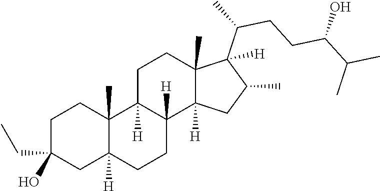 |
| B-24 | 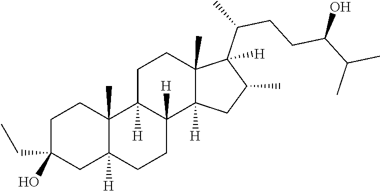 |

In one aspect, the present disclosure provides a compound of Formula (C-I):

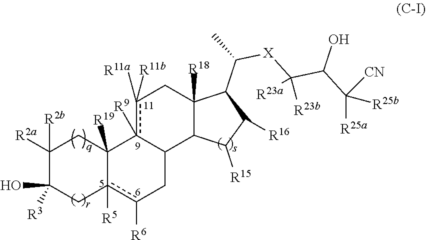

or a pharmaceutically acceptable salt thereof;
wherein:
   each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein R^{A2} is hydrogen or substituted or unsubstituted C₁₋₆alkyl;
   R³ is substituted or unsubstituted C₁₋₆alkyl, substituted or unsubstituted C₂₋₆ alkenyl, or substituted or unsubstituted C₂₋₆alkynyl, or substituted or unsubstituted C₃₋₁₀ carbocyclyl;
   R⁵ is absent, hydrogen, halogen, or substituted or unsubstituted C₁₋₆alkyl;
   R⁶ is hydrogen, halogen, or substituted or unsubstituted C₁₋₆alkyl;
   R⁹ is absent or hydrogen; or
   R⁹ and R^{11a} are taken together with the carbon atoms to which they are attached to form a substituted or unsubstituted C₃₋₆carbocyclyl ring;
   R^{11a} is hydrogen, halogen, substituted or unsubstituted C₁₋₆alkyl, or —OR^{A11}, wherein R^{A11} is hydrogen or substituted or unsubstituted C₁₋₆alkyl;
   R^{11b} is absent or hydrogen; or
   R^{11a} and R^{11b} are joined to form an oxo (═O) group;
   each instance of R¹⁵ is hydrogen, halogen, —OH, —NH₂, substituted or unsubstituted C₁₋₆alkyl, substituted or unsubstituted C₂₋₆alkenyl, substituted or unsubstituted C₂₋₆alkynyl, substituted or unsubstituted C₁₋₆alkoxy, substituted or unsubstituted C₃₋₆carbocyclyl, or substituted or unsubstituted 3- to 10-membered heterocyclyl;
   R¹⁶ is hydrogen, halogen, —OH, —NH₂, substituted or unsubstituted C₁₋₆alkyl, substituted or unsubstituted C₂₋₆alkenyl, substituted or unsubstituted C₂₋₆alkynyl, substituted or unsubstituted C₁₋₆alkoxy, substituted or unsubstituted C₃₋₆carbocyclyl, or substituted or unsubstituted 3- to 10-membered heterocyclyl; or
   R¹⁵ and R¹⁶ are taken together with the carbon atoms to which they are attached, to form a substituted or unsubstituted C₃₋₆carbocyclyl;
   R¹⁸ is hydrogen, substituted or unsubstituted C₁₋₆alkyl, or OR^{A18}, wherein each instance of R^{A18} is independently substituted or unsubstituted C₁₋₆alkyl;
   R¹⁹ is hydrogen, substituted or unsubstituted C₁₋₆alkyl, or OR^{A19}; wherein each instance of R^{A19} is independently substituted or unsubstituted C₁₋₆alkyl;
   X is —(C(R^{X})₂)ₙ— or —O—, wherein R^{X} is hydrogen or fluorine, or one R^{X} group and R^{23b} are joined to form a double bond;
n is 1, 2 or 3;
   each of R^{23a} and R^{23b} is independently hydrogen or fluorine;
   R^{25a} is hydrogen, substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, or substituted or unsubstituted 5- to 10-membered heteroaryl;
   R^{25b} is hydrogen, substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, or substituted or unsubstituted 5- to 10-membered heteroaryl; or R^{25a} and R^{25b}, taken together with the carbon atom to which they are attached, form a substituted or unsubstituted C₃₋₆carbocyclyl or a substituted or unsubstituted 3- to 10-membered heterocyclyl;
   q is 0, 1 or 2;
   r is 1 or 2;
   s is 1 or 2; and
   each [symbol] independently represents a single or double bond;
   provided if the bond between C5 and C6 is a double bond, R⁵ is absent and if the bond between C9 and C11 is a double bond, R⁹ and R^{11b} are absent.

In some embodiments, the compound of Formula (C-I) is a compound of Formula (C-II):

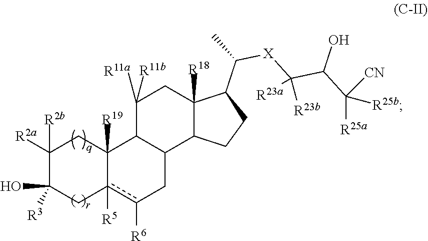

or a pharmaceutically acceptable salt thereof.

In some embodiments, the compound of Formula (C-II) is a compound of Formula (C-II-A):

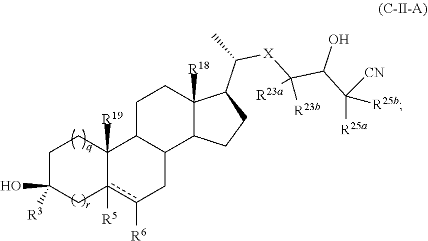

or a pharmaceutically acceptable salt thereof.

In some embodiments, the compound of Formula (C-II-A) is a compound of Formula (C-II-A1):

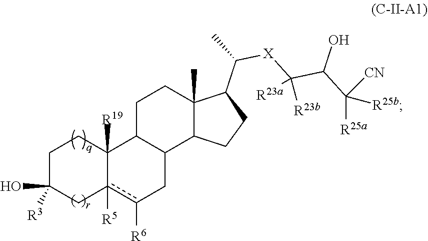

or a pharmaceutically acceptable salt thereof.

In some embodiments, the compound of Formula (C-II-A1) is a compound of Formula (C-II-A1-i):

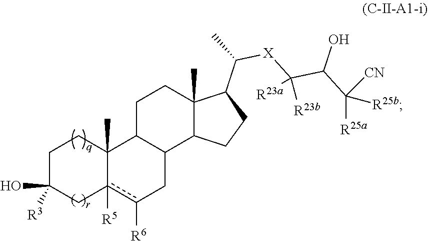

or a pharmaceutically acceptable salt thereof.

In some embodiments, the compound of Formula (C-II-A1-i) is a compound of Formula (C-II-A1-ia):

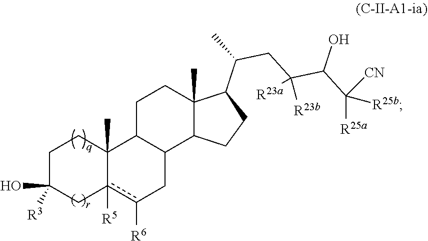

or a pharmaceutically acceptable salt thereof.

In some embodiments, the compound of Formula (C-II-A1-ia) is a compound of Formula (C-II-A1-ia-1):

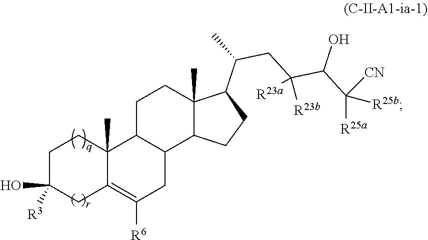

or a pharmaceutically acceptable salt thereof.

In some embodiments, the compound of Formula (C-II-A1-ia-1) is a compound of Formula (C-II-A1-ia-1a) or Formula (C-II-A1-ia-1b):

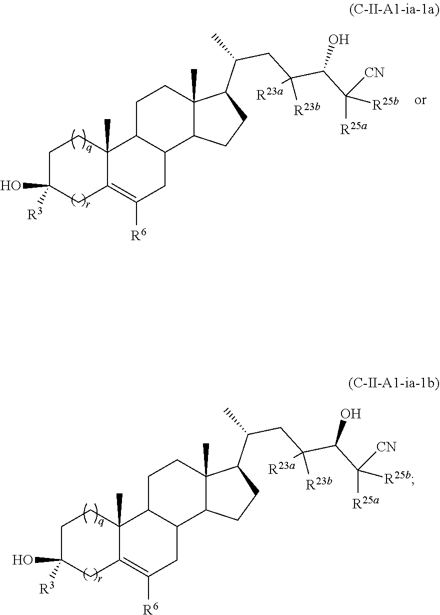

or a pharmaceutically acceptable salt thereof.

In some embodiments, the compound of Formula (C-II-A1-ia) is a compound of Formula (C-II-A1-ia-2):

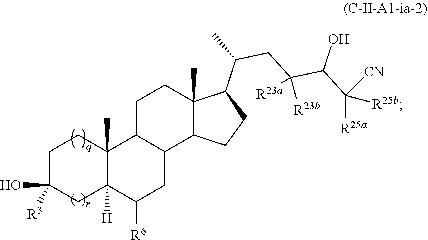

or a pharmaceutically acceptable salt thereof.

In some embodiments, the compound of Formula (C-II-A1-ia-2) is a compound of Formula (C-II-A1-ia-2a) or Formula (C-II-A1-ia-2b):

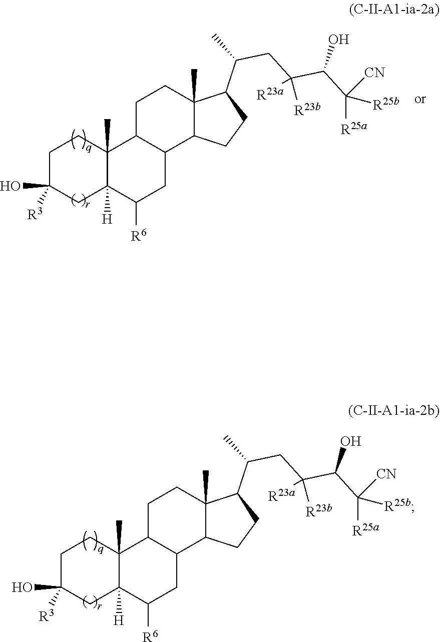

or a pharmaceutically acceptable salt thereof.

In some embodiments, the compound of Formula (C-II-A1) is a compound of Formula (C-II-A1-ii):

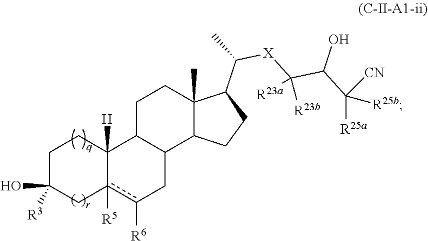

or a pharmaceutically acceptable salt thereof.

In some embodiments, the compound of Formula (C-II-A1-ii) is a compound of Formula (C-II-A1-iia):

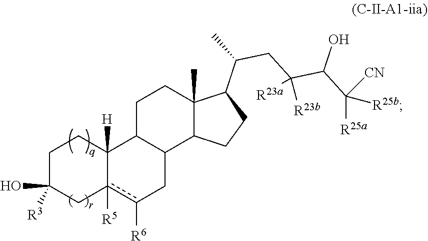

or a pharmaceutically acceptable salt thereof.

In some embodiments, the compound of Formula (C-II-A1-iia) is a compound of Formula (C-II-A1-iia-1):

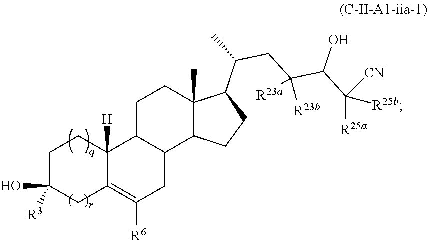

or a pharmaceutically acceptable salt thereof.

In some embodiments, the compound of Formula (C-II-A1-iia-1) is a compound of Formula (C-II-A1-iia-1a) or Formula (C-II-A1-iia-1b):

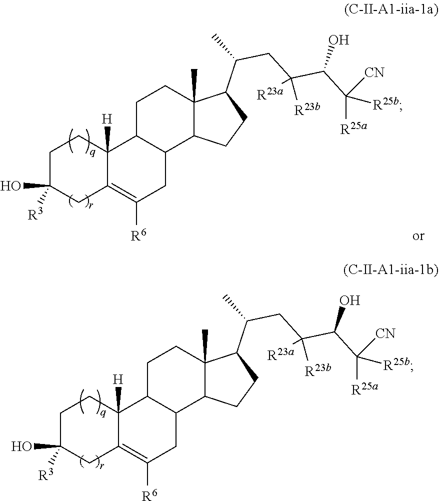

or a pharmaceutically acceptable salt thereof.

In some embodiments, the compound of Formula (C-II-A1-iia) is a compound of Formula (C-II-A1-iia-2):

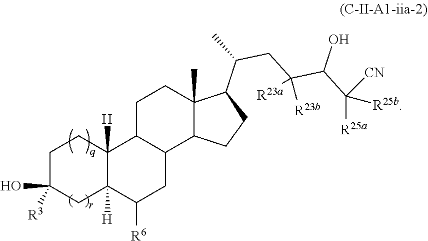

In some embodiments, the compound of Formula (C-II-A1-iia-2) is a compound of Formula (C-II-A1-iia-2a) or Formula (C-II-A1-iia-2b):

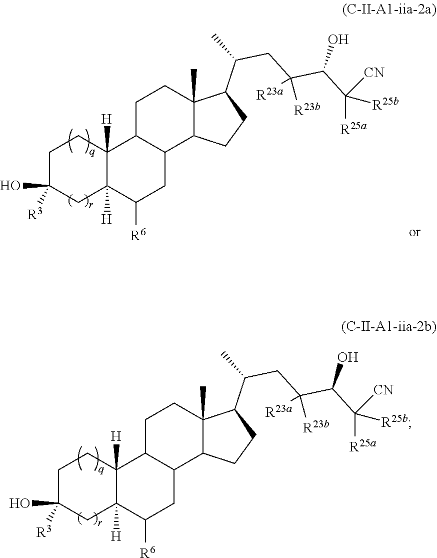

or a pharmaceutically acceptable salt thereof.

In some embodiments, the compound of Formula (C-I) is a compound of Formula (C-III):

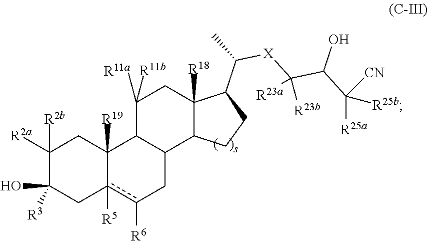

or a pharmaceutically acceptable salt thereof.

In some embodiments, the compound of Formula (C-III) is a compound of Formula (C-III-A):

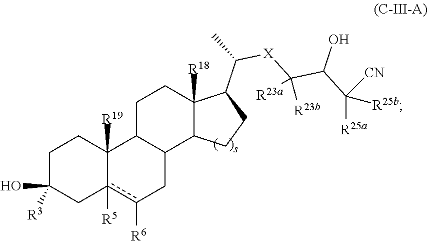

or a pharmaceutically acceptable salt thereof.

In some embodiments, the compound of Formula (C-III-A) is a compound of Formula (C-III-A1):

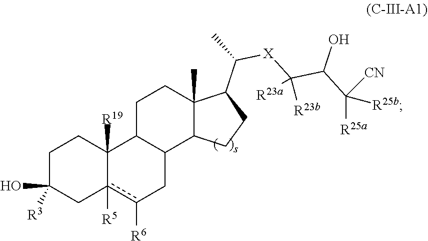

or a pharmaceutically acceptable salt thereof.

In some embodiments, the compound of Formula (C-III-A1) is a compound of Formula (C-III-A1-i):

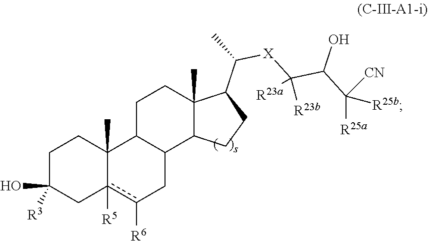

or a pharmaceutically acceptable salt thereof.

In some embodiments, the compound of Formula (C-III-A1-i) is a compound of Formula (C-III-A1-ia):

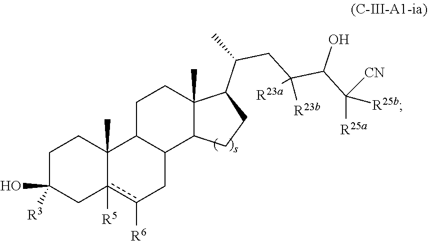

or a pharmaceutically acceptable salt thereof.

In some embodiments, the compound of Formula (C-III-A1-ia) is a compound of Formula (C-III-A1-ia-1):

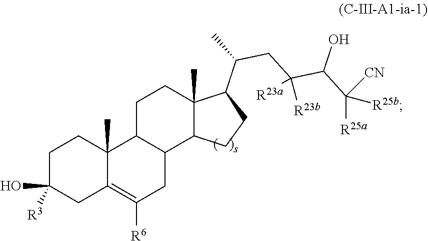

or a pharmaceutically acceptable salt thereof.

In some embodiments, the compound of Formula (C-III-A1-ia-1) is a compound of Formula (C-III-A1-ia-1a) or Formula (C-III-A1-ia-1b):

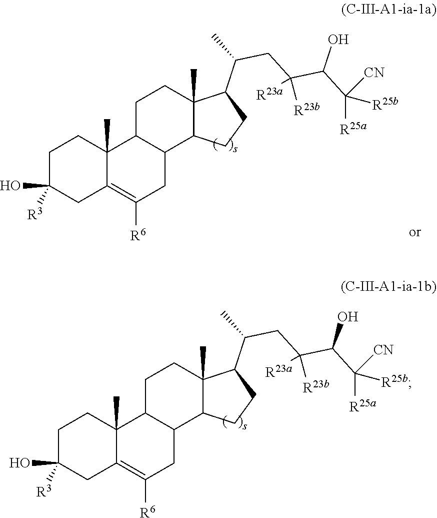

or a pharmaceutically acceptable salt thereof.

In some embodiments, the compound of Formula (C-III-A1-ia) is a compound of Formula (C-III-A1-ia-2):

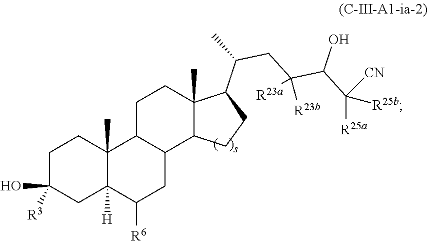

or a pharmaceutically acceptable salt thereof.

In some embodiments, the compound of Formula (C-III-A1-ia-2) is a compound of Formula (C-III-A1-ia-2a) or Formula (C-III-A1-ia-2b):

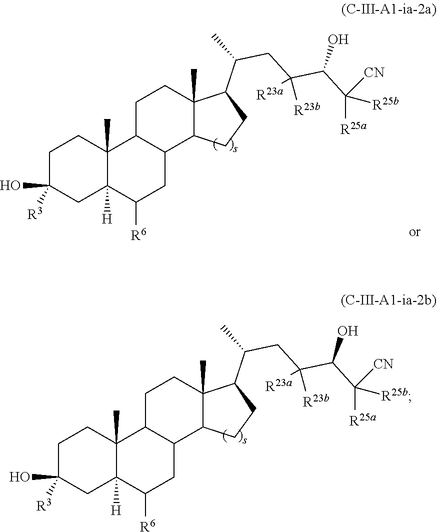

or a pharmaceutically acceptable salt thereof.

In some embodiments, the compound of Formula (C-III-A1) is a compound of Formula (C-III-A1-ii):

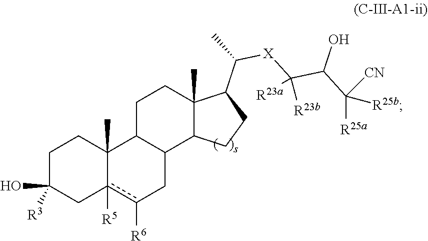

or a pharmaceutically acceptable salt thereof.

In some embodiments, the compound of Formula (C-III-A1-ii) is a compound of Formula (C-III-A1-iia):

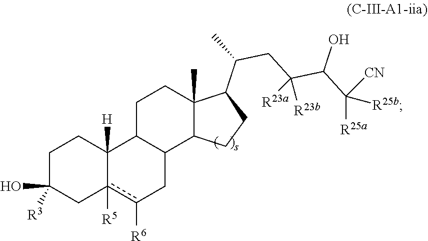

or a pharmaceutically acceptable salt thereof.

In some embodiments, the compound of Formula (C-III-A1-iia) is a compound of Formula (C-III-A1-iia-1):

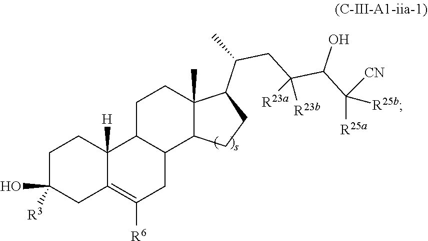

or a pharmaceutically acceptable salt thereof.

In some embodiments, the compound of Formula (C-III-A1-iia-1) is a compound of Formula (C-III-A1-iia-1a) or Formula (C-III-A1-iia-1b):

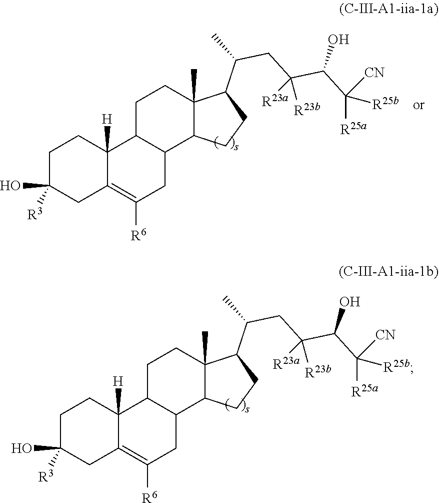

or a pharmaceutically acceptable salt thereof.

In some embodiments, the compound of Formula (C-III-A1-iia) is a compound of Formula (C-III-A1-iia-2):

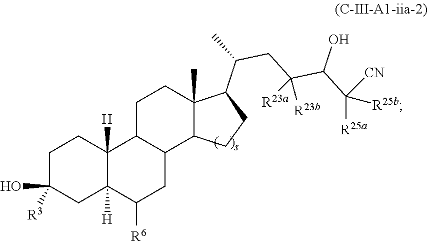

or a pharmaceutically acceptable salt thereof.

In some embodiments, the compound of Formula (C-III-A1-iia-2) is a compound of Formula (C-III-A1-iia-2a) or Formula (C-III-A1-iia-2b):

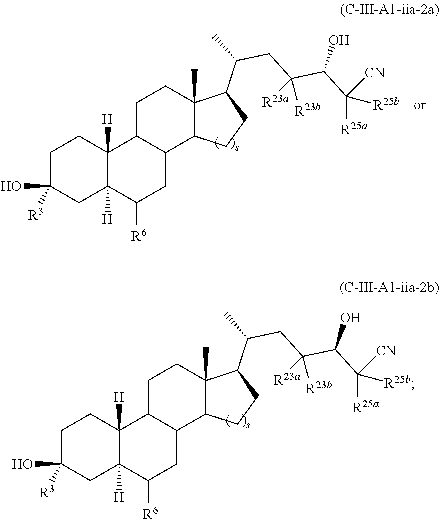

or a pharmaceutically acceptable salt thereof.

In some embodiments, the compound of Formula (C-I) is a compound of Formula (C-IV):

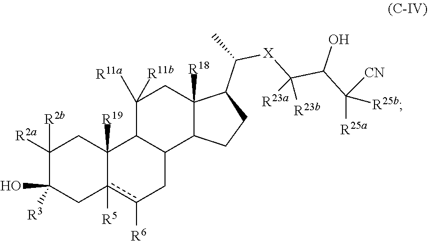

or a pharmaceutically acceptable salt thereof.

In some embodiments, the compound of Formula (C-IV) is a compound of Formula (C-IV-A):

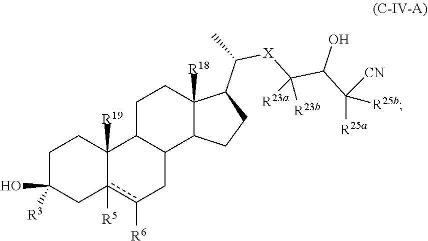

or a pharmaceutically acceptable salt thereof.

In some embodiments, the compound of Formula (C-IV-A) is a compound of Formula (C-IV-A1):

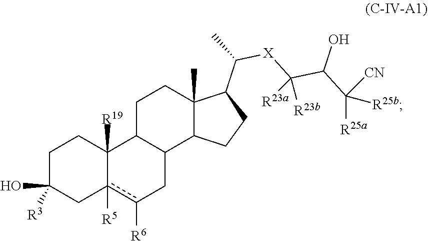

or a pharmaceutically acceptable salt thereof.

In some embodiments, the compound of Formula (C-IV-A1) is a compound of Formula (C-IV-A1-i):

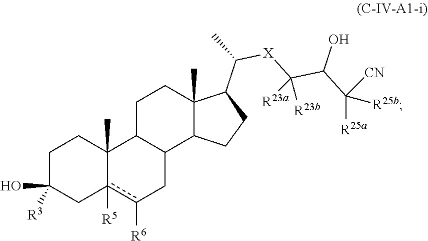

or a pharmaceutically acceptable salt thereof.

In some embodiments, the compound of Formula (C-IV-A1-i) is a compound of Formula (C-IV-A1-ia):

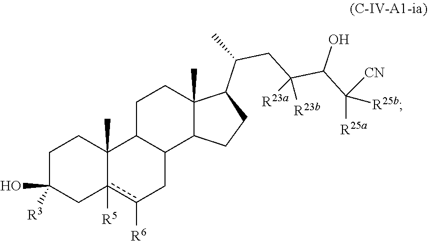

or a pharmaceutically acceptable salt thereof.

In some embodiments, the compound of Formula (C-IV-A1-ia) is a compound of Formula (C-IV-A1-ia-1):

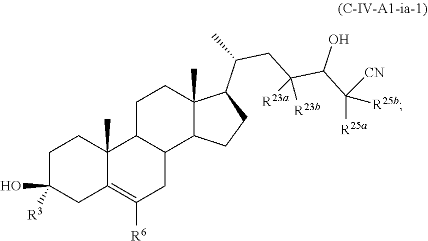

or a pharmaceutically acceptable salt thereof.

In some embodiments, the compound of Formula (C-IV-A1-ia-1) is a compound of Formula (C-IV-A1-ia-1a) or Formula (C-IV-A1-ia-1b):

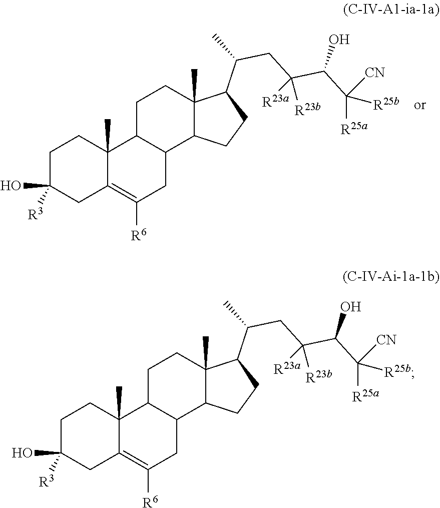

or a pharmaceutically acceptable salt thereof.

In some embodiments, the compound of Formula (C-IV-A1-ia) is a compound of Formula (C-IV-A1-ia-2):

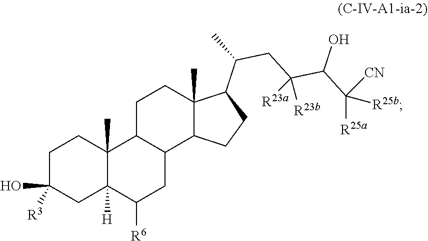

or a pharmaceutically acceptable salt thereof.

In some embodiments, the compound of Formula (C-IV-A1-ia-2) is a compound of Formula (C-IV-A1-ia-2a) or Formula (C-IV-A1-ia-2b):

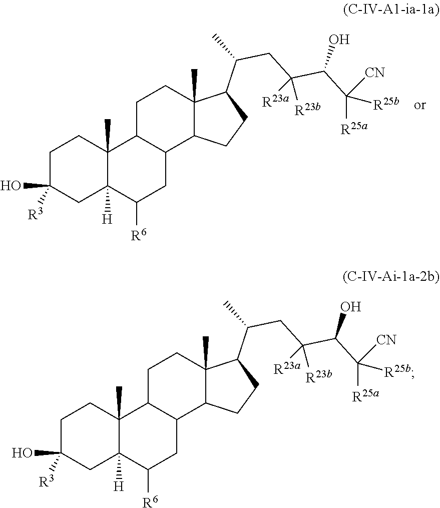

or a pharmaceutically acceptable salt thereof.

In some embodiments, the compound of Formula (C-IV-A1) is a compound of Formula (C-IV-A1-ii):

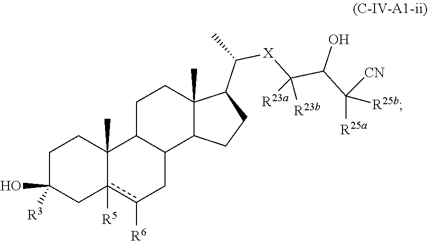

or a pharmaceutically acceptable salt thereof.

In some embodiments, the compound of Formula (C-IV-A1-ii) is a compound of Formula (C-IV-A1-iia):

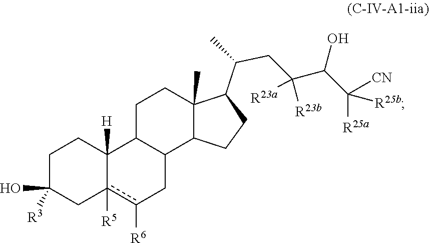

or a pharmaceutically acceptable salt thereof.

In some embodiments, the compound of Formula (C-IV-A1-iia) is a compound of Formula (C-IV-A1-iia-1):

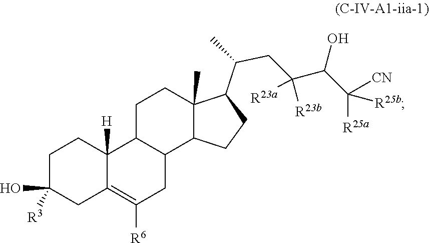

or a pharmaceutically acceptable salt thereof.

In some embodiments, the compound of Formula (C-IV-A1-iia-1) is a compound of Formula (C-IV-A1-iia-1a) or Formula (C-IV-A1-iia-1b):

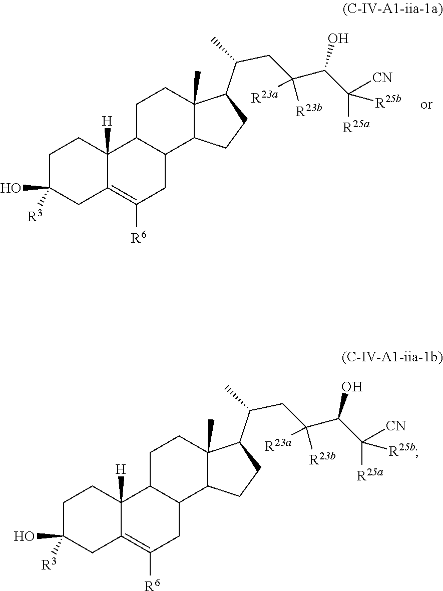

or a pharmaceutically acceptable salt thereof.

In some embodiments, the compound of Formula (C-IV-A1-iia) is a compound of Formula (C-IV-A1-iia-2):

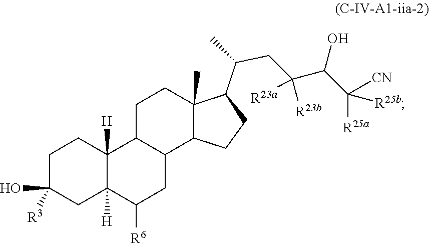

or a pharmaceutically acceptable salt thereof.

In some embodiments, the compound of Formula (C-IV-A1-iia-2) is a compound of Formula (C-IV-A1-iia-2a) or Formula (C-IV-A1-iia-2b):

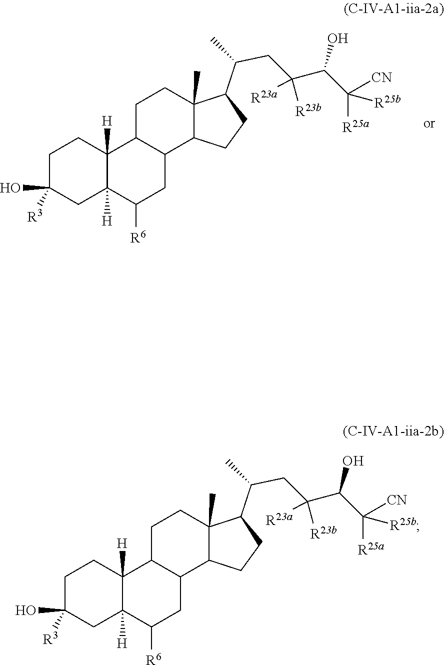

or a pharmaceutically acceptable salt thereof.

In some embodiments, the compound of Formula (C-I) is a compound of Formula (C-V):

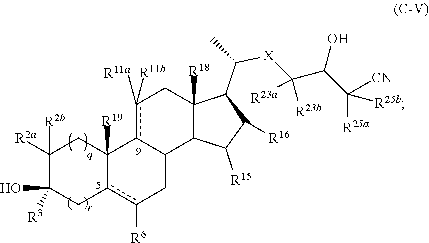

or a pharmaceutically acceptable salt thereof.

In some embodiments, the compound of Formula (C-V) is a compound of Formula (C-VA) or Formula (C-VB):

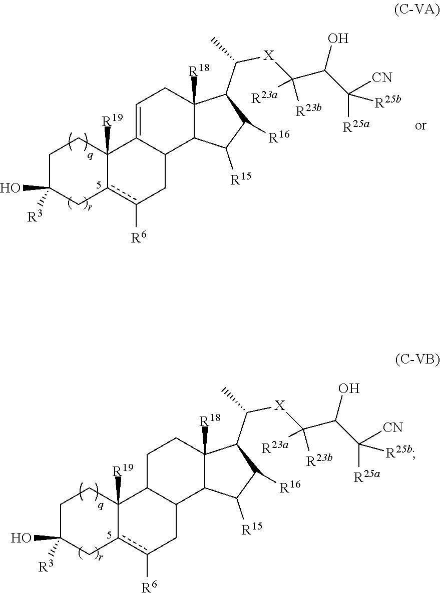

or a pharmaceutically acceptable salt thereof.

In some embodiments, the compound of Formula (C-V-A) is a compound of Formula (C-V-A1):

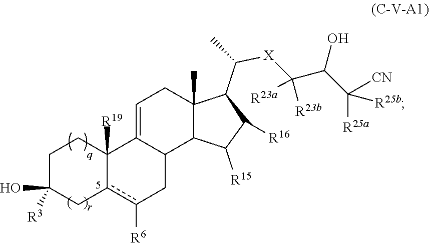

or a pharmaceutically acceptable salt thereof.

In some embodiments, the compound of Formula (C-V-A1) is a compound of Formula (C-V-A1-i):

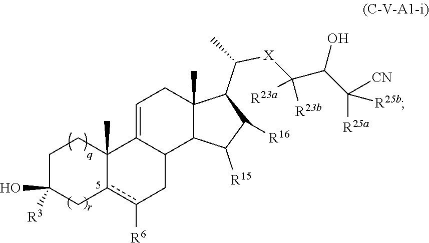

or a pharmaceutically acceptable salt thereof.

In some embodiments, the compound of Formula (C-V-A1-i) is a compound of Formula (C-V-A1-ia):

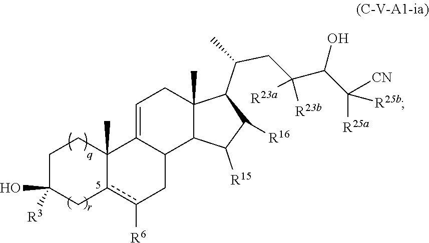

or a pharmaceutically acceptable salt thereof.

In some embodiments, the compound of Formula (C-V-A1-ia) is a compound of Formula (C-V-A1-ia-1):

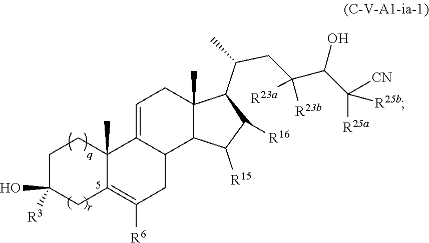

or a pharmaceutically acceptable salt thereof.

In some embodiments, the compound of Formula (C-V-A1-ia-1) is a compound of Formula (C-V-A1-ia-1a) or Formula (C-V-A1-ia-1b):

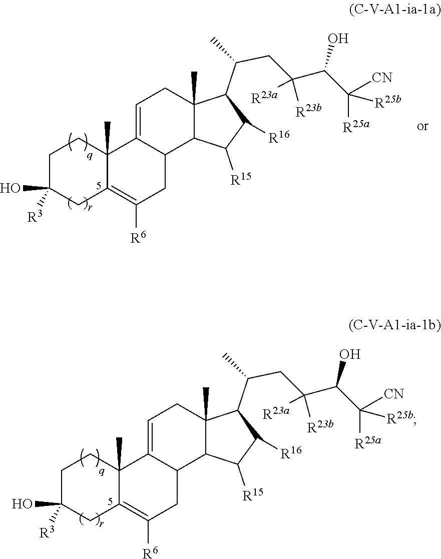

or a pharmaceutically acceptable salt thereof.

In some embodiments, the compound of Formula (C-V-A1-ia) is a compound of Formula (C-V-A1-ia-2):

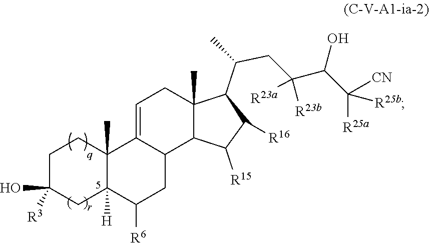

or a pharmaceutically acceptable salt thereof.

In some embodiments, the compound of Formula (C-V-A1-ia-2) is a compound of Formula (C-V-A1-ia-2a) or Formula (C-V-A1-ia-2b):

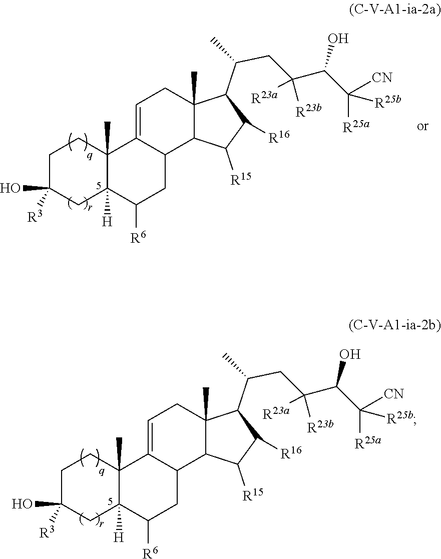

or a pharmaceutically acceptable salt thereof.

In some embodiments, the compound of Formula (C-V-A1) is a compound of Formula (C-V-A1-ii):

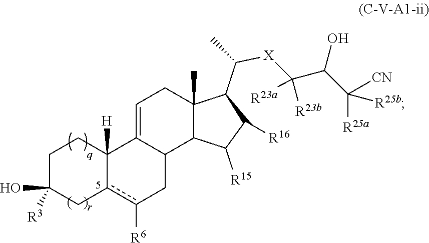

or a pharmaceutically acceptable salt thereof.

In some embodiments, the compound of Formula (C-V-A1-ii) is a compound of Formula (C-V-A1-iia):

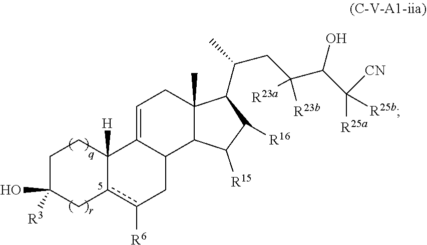

or a pharmaceutically acceptable salt thereof.

In some embodiments, the compound of Formula (C-V-A1-iia) is a compound of Formula (C-V-A1-iia-1):

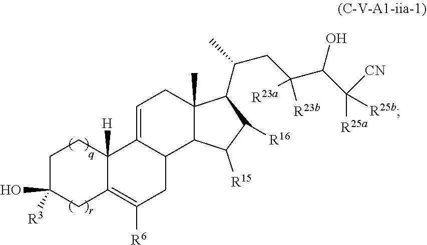

or a pharmaceutically acceptable salt thereof.

In some embodiments, the compound of Formula (C-V-A1-iia-1) is a compound of Formula (C-V-A1-iia-1a) or Formula (C-V-A1-iia-1b):

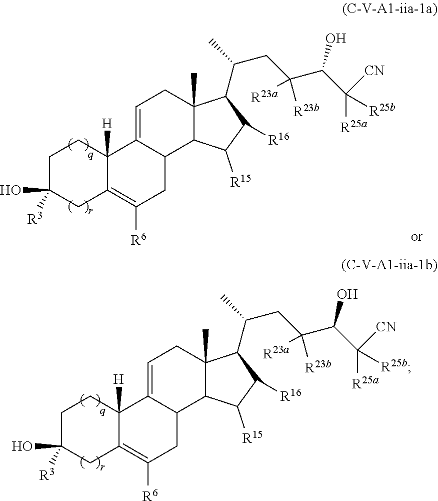

or a pharmaceutically acceptable salt thereof.

In some embodiments, the compound of Formula (C-V-A1-iia) is a compound of Formula (C-V-A1-iia-2):

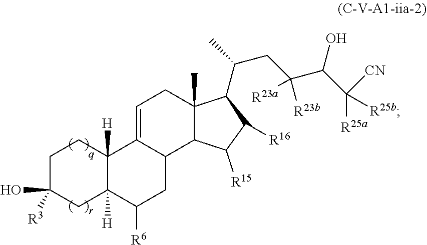

or a pharmaceutically acceptable salt thereof.

In some embodiments, the compound of Formula (C-V-A1-iia-2) is a compound of Formula (C-V-A1-iia-2a) or Formula (C-V-A1-iia-2b):

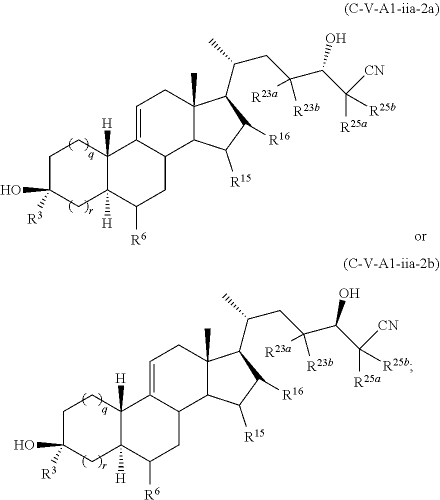

or a pharmaceutically acceptable salt thereof.

In some embodiments, the compound of Formula (C-V-B) is a compound of Formula (C-V-B1):

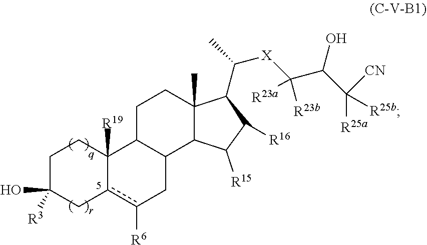

or a pharmaceutically acceptable salt thereof.

In some embodiments, the compound of Formula (C-V-B1) is a compound of Formula (C-V-B1-i):

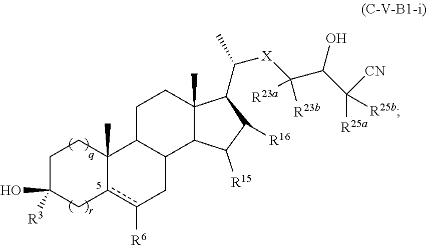

or a pharmaceutically acceptable salt thereof.

In some embodiments, the compound of Formula (C-V-B1-i) is a compound of Formula (C-V-B1-ia):

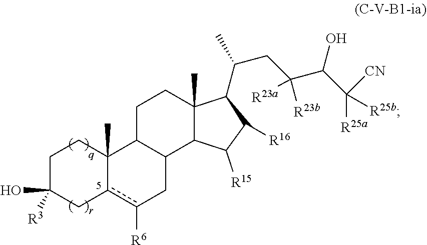

or a pharmaceutically acceptable salt thereof.

In some embodiments, the compound of Formula (C-V-B1-ia) is a compound of Formula (C-V-B1-ia-1):

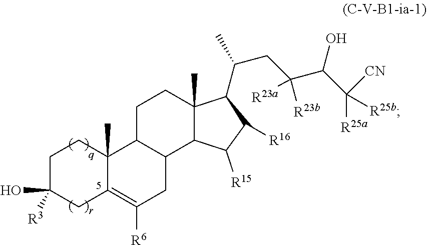

or a pharmaceutically acceptable salt thereof.

In some embodiments, the compound of Formula (C-V-B1-ia-1) is a compound of Formula (C-V-B1-ia-1a) or Formula (C-V-B1-ia-1b):

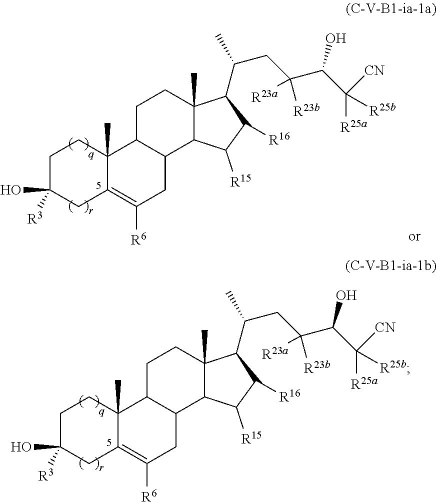

or a pharmaceutically acceptable salt thereof.

In some embodiments, the compound of Formula (C-V-B1-ia) is a compound of Formula (C-V-B1-ia-2):

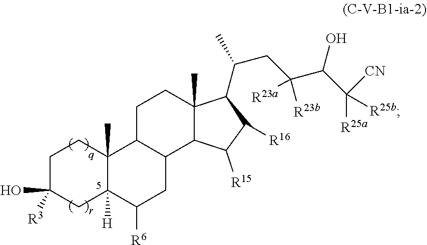

or a pharmaceutically acceptable salt thereof.

In some embodiments, the compound of Formula (C-V-B1-ia-2) is a compound of Formula (C-V-B1-ia-2a) or Formula (C-V-B1-ia-2b):

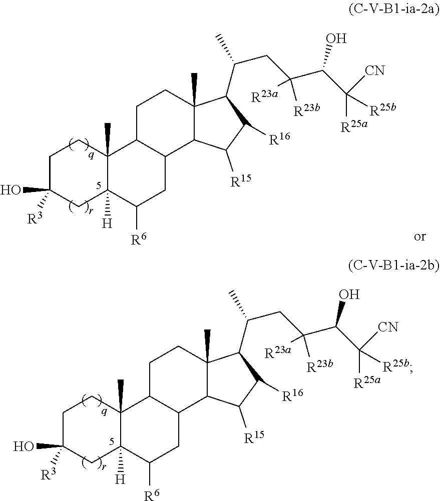

or a pharmaceutically acceptable salt thereof).

In some embodiments, the compound of Formula (C-V-B1) is a compound of Formula (C-V-B1-ii):

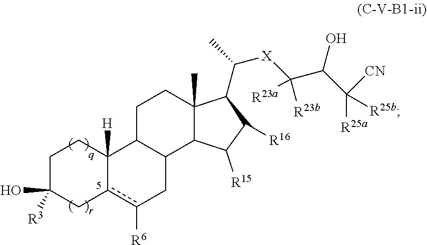

or a pharmaceutically acceptable salt thereof

In some embodiments, the compound of Formula (C-V-B1-ii) is a compound of Formula (C-V-B1-iia):

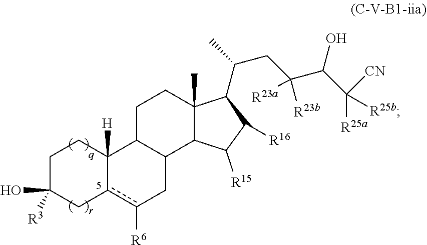

or a pharmaceutically acceptable salt thereof.

In some embodiments, the compound of Formula (C-V-B1-iia) is a compound of Formula (C-V-B1-iia-1):

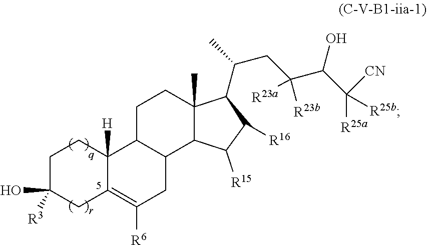

or a pharmaceutically acceptable salt thereof.

In some embodiments, the compound of Formula (C-V-B1-iia-1) is a compound of Formula (C-V-B1-iia-1a) or Formula (C-V-B1-iia-1b):

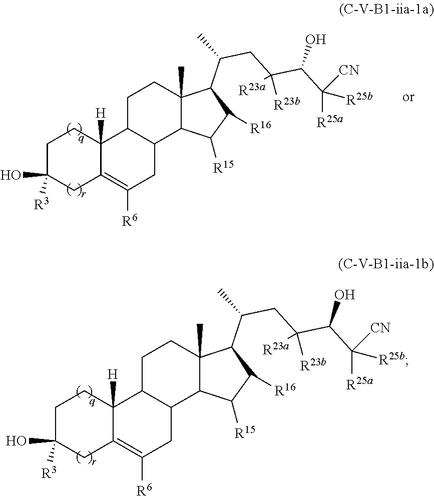

or a pharmaceutically acceptable salt thereof.

In some embodiments, the compound of Formula (C-V-B1-iia) is a compound of Formula (C-V-B1-iia-2):

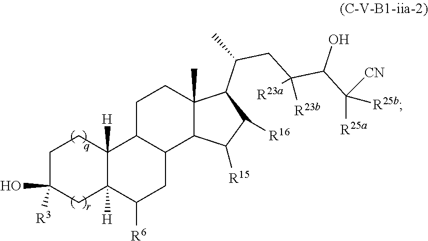

or a pharmaceutically acceptable salt thereof.

In some embodiments, the compound of Formula (C-V-B1-iia-2) is a compound of Formula (C-V-B1-iia-2a) or Formula (C-V-B1-iia-2b):

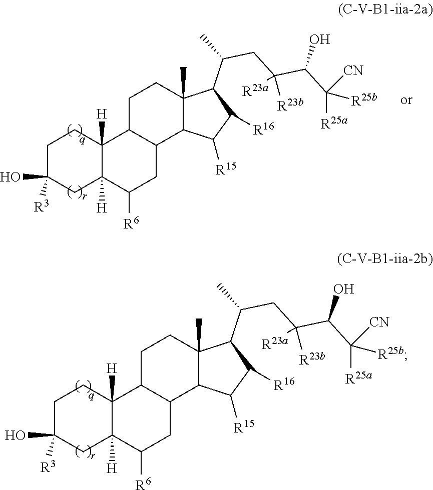

or a pharmaceutically acceptable salt thereof.

In some embodiments, the compound of Formula (C-I) is a compound of Formula (C-VI):

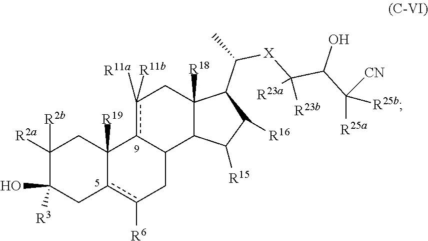

or a pharmaceutically acceptable salt thereof.

In some embodiments, the compound of Formula (C-VI) is a compound of Formula (C-VIA) or Formula (C-VIB):

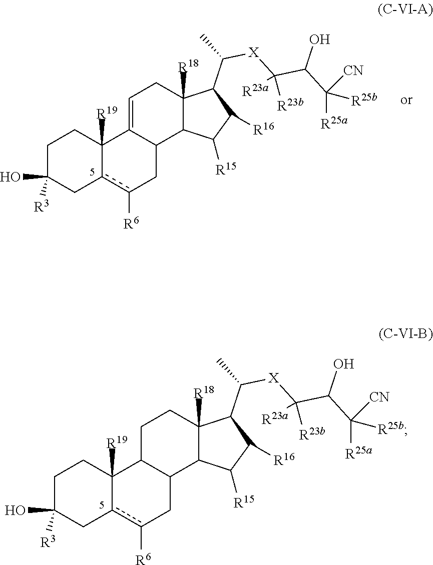

or a pharmaceutically acceptable salt thereof.

In some embodiments, the compound of Formula (C-VI-A) is a compound of Formula (C-VI-A1):

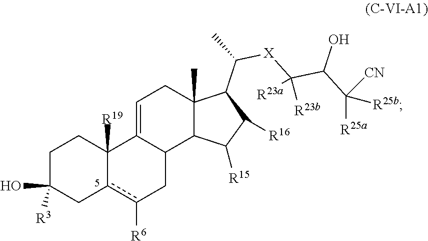

or a pharmaceutically acceptable salt thereof.

In some embodiments, the compound of Formula (C-VI-A1) is a compound of Formula (C-VI-A1-i):

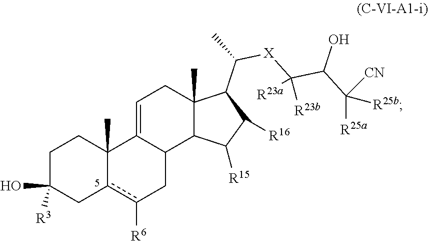

or a pharmaceutically acceptable salt thereof.

In some embodiments, the compound of Formula (C-VI-A1-i) is a compound of Formula (C-VI-A1-ia):

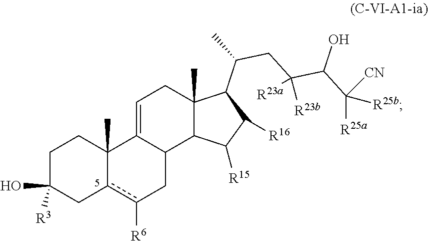

or a pharmaceutically acceptable salt thereof.

In some embodiments, the compound of Formula (C-VI-A1-ia) is a compound of Formula (C-VI-A1-ia-1):

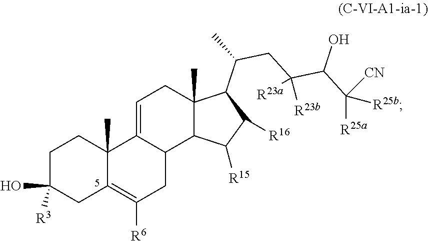

or a pharmaceutically acceptable salt thereof.

In some embodiments, the compound of Formula (C-VI-A1-ia-1) is a compound of Formula (C-VI-A1-ia-1a) or Formula (C-VI-A1-ia-1b):

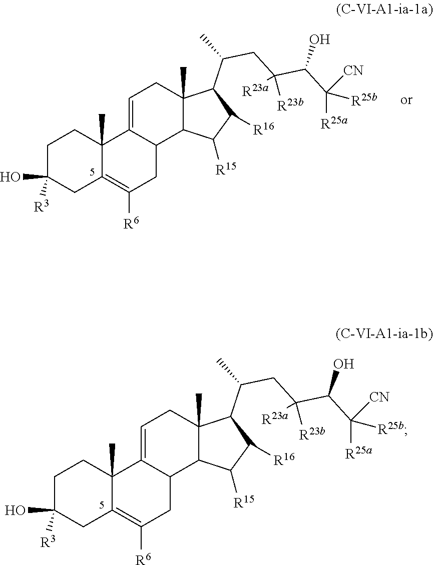

or a pharmaceutically acceptable salt thereof.

In some embodiments, the compound of Formula (C-VI-A1-ia) is a compound of Formula (C-VI-A1-ia-2):

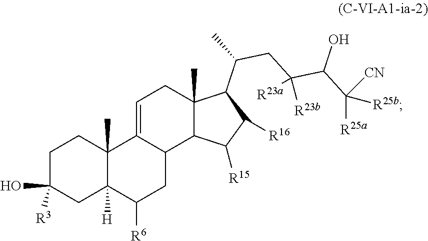

or a pharmaceutically acceptable salt thereof.

In some embodiments, the compound of Formula (C-VI-A1-ia-2) is a compound of Formula (C-VI-A1-ia-2a) or Formula (C-VI-A1-ia-2b):

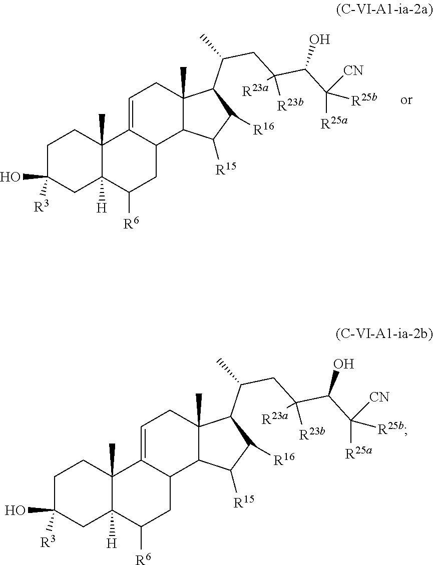

or a pharmaceutically acceptable salt thereof.

In some embodiments, the compound of Formula (C-VI-A1) is a compound of Formula (C-VI-A1-ii):

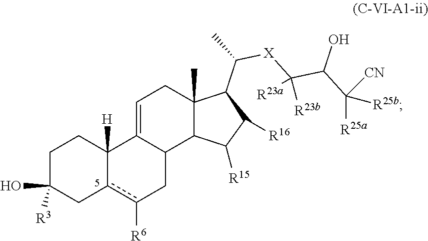

or a pharmaceutically acceptable salt thereof.

In some embodiments, the compound of Formula (C-VI-A1-ii) is a compound of Formula (C-VI-A1-iia):

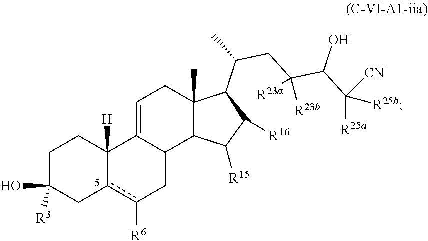

or a pharmaceutically acceptable salt thereof.

In some embodiments, the compound of Formula (C-VI-A1-iia) is a compound of Formula (C-VI-A1-iia-1):

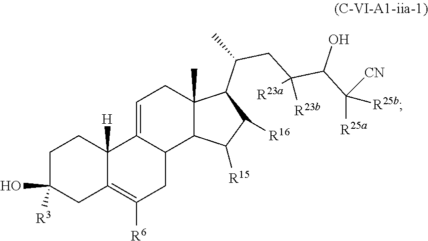

or a pharmaceutically acceptable salt thereof.

In some embodiments, the compound of Formula (C-VI-A1-iia-1) is a compound of Formula (C-VI-A1-iia-1a) or Formula (C-VI-A1-iia-1b):

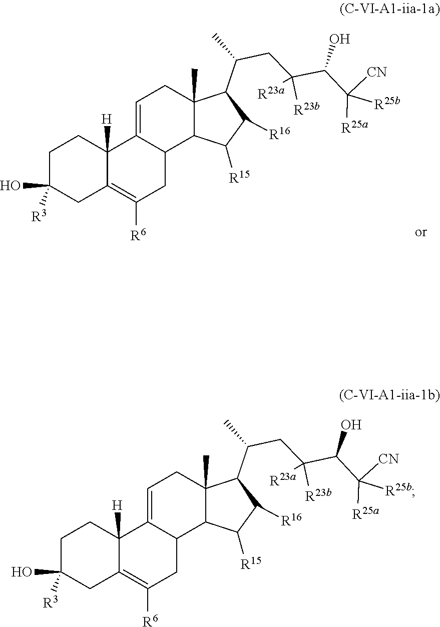

or a pharmaceutically acceptable salt thereof.

In some embodiments, the compound of Formula (C-VI-A1-iia) is a compound of Formula (C-VI-A1-iia-2):

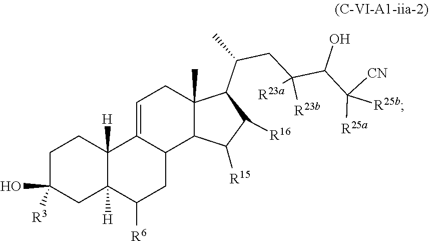

or a pharmaceutically acceptable salt thereof.

In some embodiments, the compound of Formula (C-VI-A1-iia-2) is a compound of Formula (C-VI-A1-iia-2a) or Formula (C-VI-A1-iia-2b):

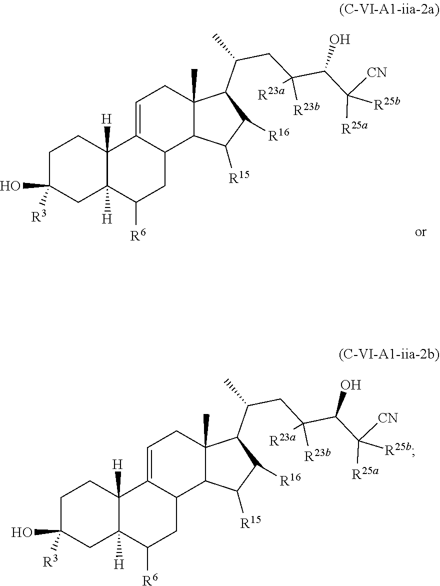

or a pharmaceutically acceptable salt thereof.

In some embodiments, the compound of Formula (C-VI-B) is a compound of Formula (C-VI-B1):

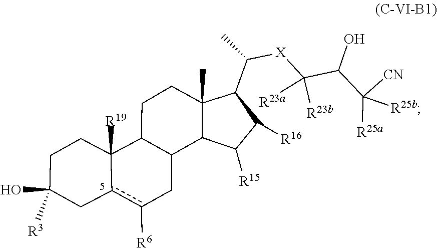

or a pharmaceutically acceptable salt thereof.

In some embodiments, the compound of Formula (C-VI-B1) is a compound of Formula (C-VI-B1-i):

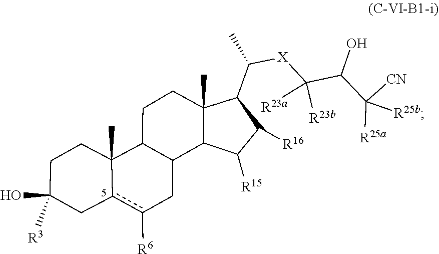

or a pharmaceutically acceptable salt thereof.

In some embodiments, the compound of Formula (C-VI-B1-i) is a compound of Formula (C-VI-B1-ia):

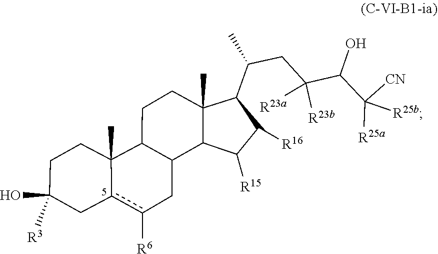

or a pharmaceutically acceptable salt thereof.

In some embodiments, the compound of Formula (C-VI-B1-ia) is a compound of Formula (C-VI-B1-ia-1):

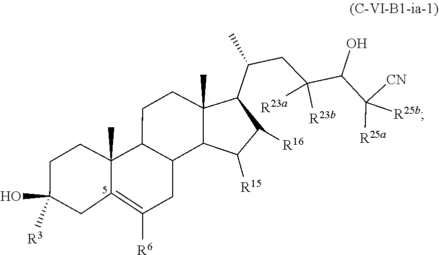

or a pharmaceutically acceptable salt thereof.

In some embodiments, the compound of Formula (C-VI-B1-ia-1) is a compound of Formula (C-VI-B1-ia-1a) or Formula (C-VI-B1-ia-1b):

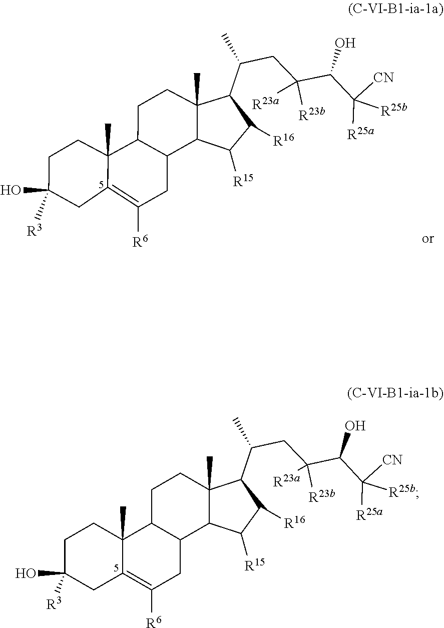

or a pharmaceutically acceptable salt thereof.

In some embodiments, the compound of Formula (C-VI-B1-ia) is a compound of Formula (C-VI-B1-ia-2):

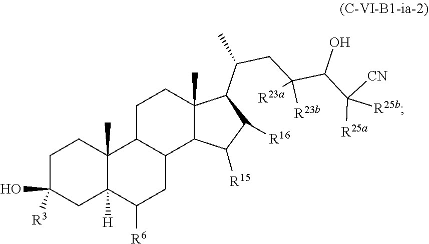

or a pharmaceutically acceptable salt thereof.

In some embodiments, the compound of Formula (C-VI-B1-ia-2) is a compound of Formula (C-VI-B1-ia-2a) or Formula (C-VI-B1-ia-2b):

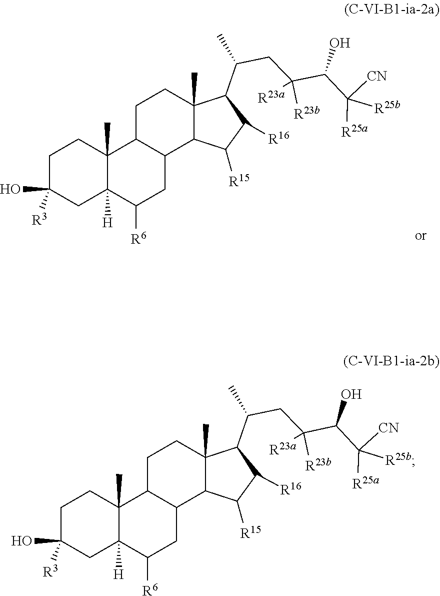

or a pharmaceutically acceptable salt thereof.

In some embodiments, the compound of Formula (C-VI-B1) is a compound of Formula (C-VI-B1-ii):

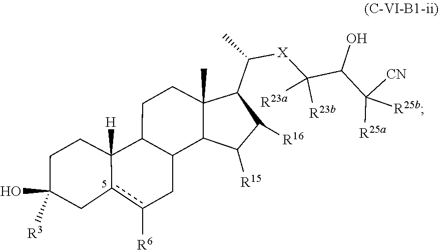

or a pharmaceutically acceptable salt thereof.

In some embodiments, the compound of Formula (C-VI-B1-ii) is a compound of Formula (C-VI-B1-iia):

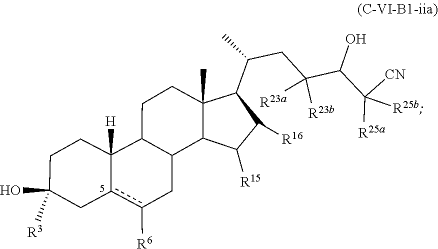

or a pharmaceutically acceptable salt thereof.

In some embodiments, the compound of Formula (C-VI-B1-iia) is a compound of Formula (C-VI-B1-iia-1):

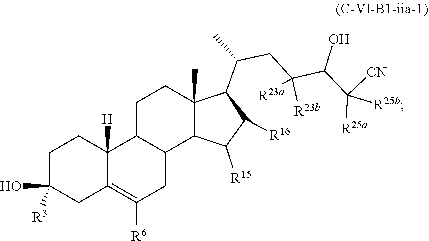

or a pharmaceutically acceptable salt thereof.

In some embodiments, the compound of Formula (C-VI-B1-iia-1) is a compound of Formula (C-VI-B1-iia-1a) or Formula (C-VI-B1-iia-1b):

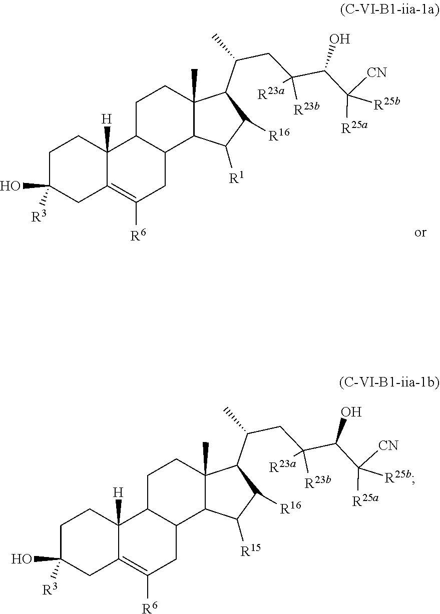

or a pharmaceutically acceptable salt thereof.

In some embodiments, the compound of Formula (C-VI-B1-iia) is a compound of Formula (C-VI-B1-iia-2):

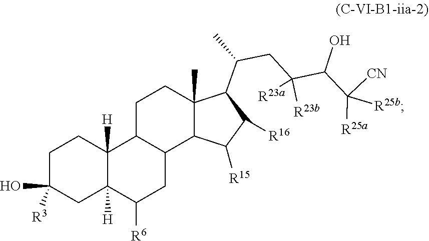

or a pharmaceutically acceptable salt thereof.

In some embodiments, the compound of Formula (C-VI-B1-iia-2) is a compound of Formula (C-VI-B1-iia-2a) or Formula (C-VI-B1-iia-2b):

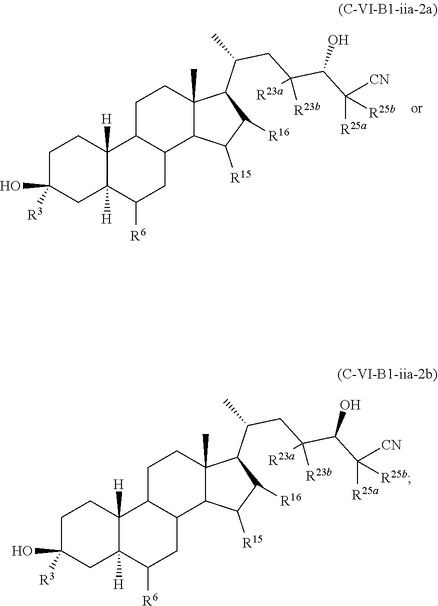

or a pharmaceutically acceptable salt thereof.
   Groups R^{2a} and R^{2b}—as they Relate to Formulae (C-I)-(C-VI-B1-iia-2b)

In certain embodiments, each of R^{2a} and R^{2b} is independently hydrogen, halogen, substituted or unsubstituted C₁₋₆alkyl, substituted or unsubstituted cyclopropyl, or —OR^{A2}, wherein R^{A2} is hydrogen or substituted or unsubstituted C₁₋₆alkyl. In certain embodiments, each of R^{2a} and R^{2b} is hydrogen. In certain embodiments, each of R^{2a} and R^{2b} is independently halogen, e.g., fluoro, chloro, bromo, or iodo. In certain embodiments, each of R^{2a} and R^{2b} is independently fluoro or chloro. In certain embodiments, each of R^{2a} and R^{2b} is independently substituted or unsubstituted C₁₋₆alkyl, e.g., substituted or unsubstituted C₁₋₂ alkyl, substituted or unsubstituted C₂₋₃ alkyl, substituted or unsubstituted C₃₋₄ alkyl, substituted or unsubstituted C₄₋₅ alkyl, or substituted or unsubstituted C₅₋₆ alkyl. In certain embodiments, each of R^{2a} and R^{2b} is independently —CH₃, —CH₂CH₃, —CH₂CH₂CH₃, or cyclopropyl. In certain embodiments, each of R^{2a} and R^{2b} is independently —OR^{A2}. In certain embodiments, R^{A2} is hydrogen. In certain embodiments, R^{A2} is substituted or unsubstituted alkyl, e.g., substituted or unsubstituted C₁₋₆alkyl, substituted or unsubstituted C₁₋₂ alkyl, substituted or unsubstituted C₂₋₃ alkyl, substituted or unsubstituted C₃₋₄ alkyl, substituted or unsubstituted C₄₋₅alkyl, or substituted or unsubstituted C₅₋₆ alkyl. In certain embodiments, R^{A2} is hydrogen, —CH₃, —CH₂CH₃, or —CH₂CH₂CH₃, i.e., to provide a group of R^{2a} or R^{2b} each independently of formula-OH, —OCH₃, —OCH₂CH₃, or —OCH₂CH₂CH₃. In certain embodiments, each of R^{2a} and R^{2b} is independently a non-hydrogen substituent in the alpha configuration. In certain embodiments, each of R^{2a} and R^{2b} is independently a non-hydrogen substituent in the beta configuration.

In some embodiments, each of R^{2a} and R^{2b} is hydrogen.

### Group R³—as it Relates to Formulae (C-1)-(C-VI-B1-iia-2b)

In certain embodiments, R³ is substituted or unsubstituted aliphatic, i.e., substituted or unsubstituted alkyl, substituted or unsubstituted alkenyl, substituted or unsubstituted alkynyl, or substituted or unsubstituted carbocyclyl.

In certain embodiments, R³ is substituted or unsubstituted C₁₋₆alkyl, substituted or unsubstituted C₂₋₆alkenyl, substituted or unsubstituted C₂₋₆alkynyl, or substituted or unsubstituted C₃₋₁₀carbocyclyl.

In certain embodiments, R³ is substituted or unsubstituted alkyl, e.g., substituted or unsubstituted C₁₋₆alkyl, substituted or unsubstituted C₁₋₂ alkyl, substituted or unsubstituted C₂₋₃ alkyl, substituted or unsubstituted C₃₋₄ alkyl, substituted or unsubstituted C₄₋₅ alkyl, or substituted or unsubstituted C₅₋₆ alkyl. Exemplary R³ C₁₋₆alkyl groups include, but are not limited to, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), n-hexyl (C₆), C₁₋₆ alkyl substituted with 1, 2, 3, 4, 5, 6, 7, 8, 9, 10, or more fluoro groups (e.g., —CF₃, —CH₂F, —CHF₂, difluoroethyl, and 2,2,2-trifluoro-1,1-dimethyl-ethyl), C₁₋₆alkyl substituted with 1, 2, 3, 4, 5, 6, 7, 8, 9, 10, or more chloro groups (e.g., —CH₂Cl, —CHCl₂), and C₁₋₆alkyl substituted with alkoxy groups (e.g., —CH₂OCH₃, —CH₂OCH₂CH₃, —CH₂O-cyclopropyl). In certain embodiments, R³ is substituted alkyl, e.g., R³ is haloalkyl, alkoxyalkyl, or aminoalkyl. In certain embodiments, R³ is Me, Et, n-Pr, n-Bu, i-Bu, fluoromethyl, chloromethyl, difluoromethyl, trifluoromethyl, trifluoroethyl, difluoroethyl, 2,2,2-trifluoro-1,1-dimethyl-ethyl, methoxymethyl, methoxyethyl, or ethoxymethyl.

In certain embodiments, R³ is substituted or unsubstituted C₁₋₆alkyl. In certain embodiments, R³ is unsubstituted C₁₋₆alkyl. In some embodiments, R³ is selected from the group consisting of CH₃, —CH₂CH₃, or —CH₂CH₂CH₃. In some embodiments, R³ is ethyl.

In certain embodiments, R³ is substituted C₁₋₆alkyl. In certain embodiments, R³ is alkyl substituted with one or more fluorine atoms; e.g., R³ is —CH₂F, —CHF₂, or —CF₃. In certain embodiments, R³ is alkyl substituted with one or more —OR^{A3} groups, wherein R^{A3} is hydrogen or substituted or unsubstituted alkyl. In certain embodiments, R³ is —CH₂OR^{A3}, e.g., wherein R^{A3} is hydrogen, —CH₃, —CH₂CH₃, or —CH₂CH₂CH₃.

In certain embodiments, R³ is —CH₃, —CH₂CH₃, —CH₂CH₂CH₃, or —CH₂OCH₃. In certain embodiments, R³ is —CH₂CH₃. In certain embodiments, R³ is —CH₂OCH₃.

In certain embodiments, R³ is substituted or unsubstituted alkenyl, e.g., substituted or unsubstituted C₂₋₆alkenyl, substituted or unsubstituted C₂₋₃alkenyl, substituted or unsubstituted C₃₋₄ alkenyl, substituted or unsubstituted C₄₋₅alkenyl, or substituted or unsubstituted C₅₋₆ alkenyl. In certain embodiments, R³ is ethenyl (C₂), propenyl (C₃), or butenyl (C₄), unsubstituted or substituted with one or more substituents selected from the group consisting of alkyl, halo, haloalkyl, alkoxyalkyl, or hydroxyl. In certain embodiments, R³ is ethenyl, propenyl, or butenyl, unsubstituted or substituted with alkyl, halo, haloalkyl, alkoxyalkyl, or hydroxy. In certain embodiments, R³ is ethenyl.

In certain embodiments, R³ is substituted or unsubstituted alkynyl, e.g., substituted or unsubstituted C₂₋₆alkynyl, substituted or unsubstituted C₂₋₃ alkynyl, substituted or unsubstituted C₃₋₄ alkynyl, substituted or unsubstituted C₄₋₅alkynyl, or substituted or unsubstituted C₅₋₆ alkynyl. Exemplary substituted or unsubstituted R³ alkynyl groups include, but are not limited to, ethynyl, propynyl, or butynyl, unsubstituted or substituted with alkyl, halo, haloalkyl (e.g., CF₃), alkoxyalkyl, cycloalkyl (e.g., cyclopropyl or cyclobutyl), or hydroxyl. In certain embodiments, R³ is selected from the group consisting of trifluoroethynyl, cyclopropylethynyl, cyclobutylethynyl, and propynyl, fluoropropynyl, and chloroethynyl. In certain embodiments, R³ is ethynyl (C₂), propynyl (C₃), or butynyl (C₄), unsubstituted or substituted with one or more substituents selected from the group consisting of substituted or unsubstituted aryl, substituted or unsubstituted heteroaryl, substituted or unsubstituted carbocyclyl, and substituted or unsubstituted heterocyclyl. In certain embodiments, R³ is ethynyl (C₂), propynyl (C₃), or butynyl (C₄) substituted with substituted phenyl. In certain embodiments, the phenyl substituent is further substituted with one or more substituents selected from the group consisting of halo, alkyl, trifluoroalkyl, alkoxy, acyl, amino or amido. In certain embodiments, R³ is ethynyl (C₂), propynyl (C₃), or butynyl (C₄) substituted with substituted or unsubstituted pyrrolyl, imidazolyl, pyrazolyl, oxazoyl, thiazolyl, isoxazoyl, 1,2,3-triazolyl, 1,2,4-triazolyl, oxadiazolyl, thiadiazolyl, or tetrazolyl.

In certain embodiments, R³ is ethynyl, propynyl, or butynyl, unsubstituted or substituted with alkyl, halo, haloalkyl, alkoxyalkyl, or hydroxyl. In certain embodiments, R³ is ethynyl or propynyl, substituted with substituted or unsubstituted aryl. In certain embodiments, R³ is ethynyl or propynyl, substituted with phenyl unsubstituted or substituted with halo, alkyl, alkoxy, haloalkyl, trihaloalkyl, or acyl. In certain embodiments, R³ is ethynyl or propynyl, substituted with substituted or unsubstituted carbocyclyl. In certain embodiments, R³ is ethynyl or propynyl, substituted with substituted or unsubstituted cyclopropyl, cyclobutyl, cyclopentyl, or cyclohexyl. In certain embodiments, R³ is ethynyl or propynyl, substituted with substituted or unsubstituted heteroaryl. In certain embodiments, R³ is ethynyl or propynyl, substituted with substituted or unsubstituted pyridinyl, or pyrimidinyl. In certain embodiments, R³ is ethynyl or propynyl, substituted with substituted or unsubstituted pyrrolyl, imidazolyl, pyrazolyl, oxazoyl, thiazolyl, isoxazoyl, 1,2,3-triazolyl, 1,2,4-triazolyl, oxadiazolyl, thiadiazolyl, tetrazolyl. In certain embodiments, R³ is ethynyl or propynyl, substituted with substituted or unsubstituted heterocyclyl. In certain embodiments, R³ is ethynyl or propynyl, substituted with substituted or unsubstituted pyrrolidinyl, piperidinyl, piperazinyl, or mopholinyl. In certain embodiments, R³ is propynyl or butynyl, substituted with hydroxyl or alkoxy. In certain embodiments, R³ is propynyl or butynyl, substituted with methoxy or ethoxy. In certain embodiments, R³ is ethynyl or propynyl, substituted with chloro. In certain embodiments, R³ is ethynyl or propynyl, substituted with trifluoromethyl.

In certain embodiments, R³ is substituted or unsubstituted carbocyclyl, e.g., substituted or unsubstituted C₃₋₁₀carbocyclyl, substituted or unsubstituted C₃₋₆carbocyclyl, substituted or unsubstituted C₃₋₄ carbocyclyl, substituted or unsubstituted C₄₋₅ carbocyclyl, or substituted or unsubstituted C₅₋₆ carbocyclyl. In certain embodiments, R³ is substituted or unsubstituted cyclopropyl or substituted or unsubstituted cyclobutyl.

Groups R⁵ and R⁶—as they Relate to Formulae (C-I)-(C-VI-B1-iia-2b)

In some embodiments, R⁵ is in the alpha or beta configuration. In certain embodiments, R⁵ is in the alpha configuration. In certain embodiments, R⁵ is in the beta configuration. In some embodiments, when the bond between C5 and C6 is a double bond, R⁵ is absent. In some embodiments, the bond between C5 and C6 is a single bond and R⁵ is hydrogen in the alpha configuration.

In certain embodiments, R⁵ is absent, hydrogen, halogen, or substituted or unsubstituted C₁₋₆alkyl. In some embodiments, R⁵ is halogen. In some embodiments, R⁵ is substituted C₁₋₆alkyl. In certain embodiments, R⁵ is substituted or unsubstituted alkyl, e.g., substituted or unsubstituted C₁₋₆alkyl, substituted or unsubstituted C₁₋₂ alkyl, substituted or unsubstituted C₂₋₃ alkyl, substituted or unsubstituted C₃₋₄ alkyl, substituted or unsubstituted C₄₋₅alkyl, or substituted or unsubstituted C₅₋₆ alkyl. In some embodiments, R⁵ is substituted or unsubstituted C₁₋₆alkyl. In some embodiments, R⁵ is unsubstituted C₁₋₆alkyl. In some embodiments, R⁵ is unsubstituted C₁ alkyl. In some embodiments, R⁵ is —CH₃.

In certain embodiments, R⁶ is hydrogen, halogen, or substituted or unsubstituted C₁₋₆ alkyl. In certain embodiments, R⁶ is hydrogen. In certain embodiments, R⁶ is halogen, e.g., fluoro. In certain embodiments, R⁶ is substituted or unsubstituted alkyl, e.g., substituted or unsubstituted C₁₋₆alkyl, substituted or unsubstituted C₁₋₂ alkyl, substituted or unsubstituted C₂₋₃ alkyl, substituted or unsubstituted C₃₋₄ alkyl, substituted or unsubstituted C₄₋₅ alkyl, or substituted or unsubstituted C₅₋₆ alkyl. In certain embodiments, R⁶ is —CH₃ or —CF₃. In certain embodiments, R⁶ is hydrogen, —CH₃, or —F.

Groups R⁹, R^{11a}, and R^{11b}—as they Relate to Formulae (C-I)-(C-VI-B1-iia-2b)

In certain embodiments, R⁹ is absent or hydrogen. In some embodiments, R⁹ is hydrogen. In some embodiments, when the bond between C9 and C11 is a double bond, R⁹ is absent. In some embodiments, the bond between C9 and C11 is a double bond, R⁹ is absent and R^{11a} is selected from the group consisting of hydrogen, halogen, substituted or unsubstituted C₁₋₆alkyl, and —OR^{A11}, wherein R^{A11} is hydrogen or substituted or unsubstituted C₁₋₆alkyl, and R^{11b} is absent or hydrogen. In some embodiments, the bond between C9 and C11 is a single bond, R⁹ is hydrogen and R^{11a} is selected from the group consisting of hydrogen, halogen, substituted or unsubstituted C₁₋₆alkyl, or —OR^{A11}, wherein R^{A11} is hydrogen or substituted or unsubstituted C₁₋₆alkyl, and R^{11b} is absent or hydrogen.

In certain embodiments, R^{11a} is hydrogen, halogen, substituted or unsubstituted C₁₋₆ alkyl, or —OR^{A11}, wherein R^{A11} is hydrogen or substituted or unsubstituted C₁₋₆alkyl, and R^{11b} is absent or hydrogen; or R^{11a} and R^{11b} are joined to form an oxo (═O) group.

In certain embodiments, R^{11a} and R^{11b} are joined to form an oxo (═O) group.

In certain embodiments, R^{11a} is —OR^{A11} and R^{11b} is hydrogen. In certain embodiments, wherein R^{11a} is —OR^{A11}, R^{11a} is in the alpha or beta configuration. In certain embodiments, wherein R^{11a} is —OR^{A11}, R^{11a} is in the alpha configuration. In certain embodiments, wherein R^{11a} is —OR^{A11}, R^{11a} is in the beta configuration. In certain embodiments, R^{A11} is hydrogen. In certain embodiments, R^{A11} is substituted or unsubstituted alkyl, e.g., substituted or unsubstituted C₁₋₆alkyl, substituted or unsubstituted C₁₋₂ alkyl, substituted or unsubstituted C₂₋₃ alkyl, substituted or unsubstituted C₃₋₄alkyl, substituted or unsubstituted C₄₋₅alkyl, or substituted or unsubstituted C₅₋₆ alkyl. In certain embodiments, R^{A11} is hydrogen, —CH₃, —CH₂CH₃, or —CH₂CH₂CH₃, i.e., to provide a group R^{11a} of formula —OH, —OCH₃, —OCH₂CH₃, or —OCH₂CH₂CH₃.

In some embodiments, R^{11b} is absent or hydrogen. In some embodiments, R^{11b} is hydrogen. In some embodiments, the bond between C9 and C11 is a double bond, and R^{11b} is absent.

In some embodiments, each of R^{11a} and R^{11b} is hydrogen.

In some embodiments, R⁹ and R^{11a} are taken together with the carbon atoms to which they are attached to form a substituted or unsubstituted C₃₋₆carbocyclyl.

Groups R¹⁵ and R¹⁶—as they Relate to Formulae (C-I)-(C-VI-B1-iia-2b)

In certain embodiments, R¹⁵ is hydrogen, halogen, —OH, —NH₂, substituted or unsubstituted C₁₋₆alkyl, substituted or unsubstituted C₂₋₆alkenyl, substituted or unsubstituted C₂₋₆alkynyl, substituted or unsubstituted C₁₋₆alkoxy, substituted or unsubstituted C₃₋₆carbocyclyl, or substituted or unsubstituted 3- to 10-membered heterocyclyl. In some embodiments, R¹⁵ is hydrogen, substituted or unsubstituted C₁₋₆alkyl or substituted or unsubstituted C₃₋₆carbocyclyl. In certain embodiments, R¹⁵ is hydrogen. In some embodiments, R¹⁵ is substituted or unsubstituted C₁₋₆alkyl. In some embodiments, R¹⁵ is unsubstituted C₁₋₆alkyl. In some embodiments, R¹⁵ is —CH₃. In some embodiments, R¹⁵ is substituted or unsubstituted C₃₋₆carbocyclyl. In some embodiments, R¹⁵ is substituted with substituted or unsubstituted cyclopropyl, cyclobutyl, cyclopentyl, or cyclohexyl. In some embodiments, R¹⁵ is unsubstituted cyclopropyl. In some embodiments, R¹⁵ is hydrogen, —CH₃ or cyclopropyl.

In certain embodiments, R¹⁶ is hydrogen, halogen, —OH, —NH₂, substituted or unsubstituted C₁₋₆alkyl, substituted or unsubstituted C₂₋₆alkenyl, substituted or unsubstituted C₂₋₆alkynyl, substituted or unsubstituted C₁₋₆alkoxy, substituted or unsubstituted C₃₋₆carbocyclyl, or substituted or unsubstituted 3- to 10-membered heterocyclyl. In some embodiments, R¹⁶ is hydrogen or unsubstituted C₁₋₆alkyl. In some embodiments, R¹⁶ is hydrogen or —CH₃. In some embodiments, R¹⁶ is hydrogen. In certain embodiments, R¹⁶ is substituted or unsubstituted C₁₋₆alkyl. In some embodiments, R¹⁶ is unsubstituted C₁₋₆alkyl. In some embodiments, R¹⁶ is —CH₃.

In certain embodiments, R¹⁵ and R¹⁶ are taken together with the carbon atoms to which they are attached to form a substituted or unsubstituted C₃₋₆carbocyclyl. In some embodiments, R¹⁵ and R¹⁶ are taken together with the carbon atoms to which they are attached to form a substituted or unsubstituted cyclopropyl, cyclobutyl, cyclopentyl, or cyclohexyl. In some embodiments, R¹⁵ and R¹⁶ are taken together with the carbon atoms to which they are attached to form an unsubstituted cyclopropyl, cyclobutyl, cyclopentyl, or cyclohexyl. In some embodiments, R¹⁵ and R¹⁶ are taken together with the carbon atoms to which they are attached to form an unsubstituted cyclopropyl.

Groups R¹⁸ and R¹⁹—as they Relate to Formulae (C-I)-(C-VI-B1-iia-2b)

In some embodiments R¹⁸ is hydrogen, substituted or unsubstituted C₁₋₆alkyl, or OR^{A18}, wherein each instance of R^{A18} is independently substituted or unsubstituted C₁₋₆ alkyl. In some embodiments, R¹⁸ is hydrogen, unsubstituted C₁₋₆alkyl, C₁₋₆alkyl substituted with OR^{A18}, or —OR^{A18}, wherein each instance of R^{A18} is independently substituted or unsubstituted C₁₋₆alkyl. In some embodiments, R¹⁸ is hydrogen, unsubstituted C₁₋₆alkyl, C₁₋₆alkyl substituted with OR^{A18}, or —OR^{A18}, wherein each instance of R^{A18} is independently unsubstituted C₁₋₆alkyl. In some embodiments, R¹⁸ is unsubstituted C₁₋₆alkyl. In some embodiments, R¹⁸ is —CH₃, —CH₂CH₃, or —CH(CH₃)₂. In some embodiments, R¹⁸ is —CH₃. In some embodiments, R¹⁸ is —CH₂CH₃, or —CH(CH₃)₂. In some embodiments, R¹⁸ is substituted C₁₋₆alkyl. In some embodiments, R¹⁸ is C₁₋₆alkyl substituted with one or more OR^{A18}, wherein each instance of R^{A18} is independently substituted or unsubstituted C₁₋₆ alkyl. In some embodiments, R¹⁸ is C₁₋₆alkyl substituted with OR^{A18}, wherein R^{A18} is substituted or unsubstituted C₁₋₆alkyl. In some embodiments, R¹⁸—OR^{A18}, wherein R^{A18} is substituted or unsubstituted C₁₋₆alkyl. In some embodiments, R¹⁸ is hydrogen, —CH₃, —CH₂OCH₃, —OCH₃, —CH₂CH₃, or —CH(CH₃)₂. In some embodiments, R¹⁸ is hydrogen, —CH₂OCH₃, —OCH₃, —CH₂CH₃, or —CH(CH₃)₂. In some embodiments, R¹⁸ is hydrogen or —CH₃. In some embodiments, R¹⁸ is hydrogen. In some embodiments, R¹⁸ is —CH₃. In some embodiments, R¹⁸ is —CH₂CH₃.

In some embodiments R¹⁹ is hydrogen, substituted or unsubstituted C₁₋₆alkyl, or OR^{A19}; wherein each instance of R^{A19} is independently substituted or unsubstituted C₁₋₆alkyl. In some embodiments, R¹⁹ is hydrogen, unsubstituted C₁₋₆alkyl, C₁₋₆alkyl substituted with OR^{A19}, or —OR^{A19}; wherein each instance of R^{A19} is independently substituted or unsubstituted C₁₋₆alkyl. In some embodiments, R¹⁹ is hydrogen, unsubstituted C₁₋₆alkyl, C₁₋₆alkyl substituted with OR^{A19}, or —OR^{A19}; wherein each instance of R^{A19} is independently unsubstituted C₁₋₆alkyl. In some embodiments, R¹⁹ is unsubstituted C₁₋₆alkyl. In some embodiments, R¹⁹ is —CH₃, —CH₂CH₃, or —CH(CH₃)₂. In some embodiments, R¹⁹ is —CH₃. In some embodiments, R¹⁹ is —CH₂CH₃. In some embodiments, R¹⁹ is —CH₂CH₃, or CH(CH₃)₂. In some embodiments, R¹⁹ is substituted C₁₋₆alkyl. In some embodiments, R¹⁹ is C₁₋₆alkyl substituted with one or more OR^{A19}; wherein each instance of R^{A19} is independently substituted or unsubstituted C₁₋₆alkyl. In some embodiments, R¹⁹ is C₁₋₆alkyl substituted with OR^{A19}, wherein R^{A19} is substituted or unsubstituted C₁₋₆alkyl. In some embodiments, R¹⁹ is —OR^{A19}; wherein R^{A19} is substituted or unsubstituted C₁₋₆alkyl. In some embodiments, R¹⁹ is hydrogen, —CH₃, —CH₂OCH₃, —OCH₃, —CH₂CH₃, or —CH(CH₃)₂. In some embodiments, R¹⁹ is hydrogen, —CH₂OCH₃, —OCH₃, —CH₂CH₃, or —CH(CH₃)₂. In some embodiments, R¹⁹ is hydrogen, —CH₃, or —CH₂CH₃. In some embodiments R¹⁹ is hydrogen or —CH₃. In some embodiments, R¹⁹ is hydrogen.

Groups X, n, R^{23a}, R^{23b}, R^{25a}, and R^{25b}—as they Relate to Formulae (C-I)-(C-VI-B1-iia-2b)

In certain embodiments X is —(C(R^{X})₂)ₙ— or —O—, wherein R^{X} is hydrogen or fluorine, or one R^{X} group and R^{23b} are joined to form a double bond.

In certain embodiments, X is —O—. In certain embodiments, X is —CH₂—. In certain embodiments, X is —CF₂—.

In some embodiments, X is —(C(R^{X})₂—, wherein R^{X} is hydrogen or fluorine. In some embodiments, X is —CH₂—.

In certain embodiments, n is an integer selected from 1, 2 or 3. In some embodiments, n is 1. In some embodiments, n is 2. In some embodiments, n is 3.

In some embodiments, each of R^{23a} and R^{23b} is independently hydrogen. In some embodiments, each of R^{23a} and R^{23b} is independently fluorine. In certain embodiments, at least one of R^{23a} and R^{23b} is fluorine. In certain embodiments, R^{X} and R^{23b} are joined to form a double bond, e.g., cis or trans double bond.

In some embodiments, R^{25a} is selected from the group consisting of hydrogen, substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl.

In some embodiments, R^{25b} is hydrogen, substituted or unsubstituted C₁₋₆alkyl group, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, or substituted or unsubstituted 5- to 10-membered heteroaryl.

In some embodiments, R^{25a} is hydrogen or substituted or unsubstituted C₁₋₆alkyl. In some embodiments, R^{25a} is hydrogen or unsubstituted C₁₋₆alkyl. In some embodiments, R^{25a} is unsubstituted C₁₋₆alkyl. In some embodiments, R^{25a} is —CH₃, or —CH₂CH₃. In some embodiments, R^{25a} is —CH₃. In some embodiments, R^{25a} is —CH₂CH₃. In some embodiments, R^{25a} is hydrogen.

In some embodiments, R^{25b} is hydrogen or substituted or unsubstituted C₁₋₆alkyl. In some embodiments, R^{25b} is hydrogen or unsubstituted C₁₋₆alkyl. In some embodiments, R^{25b} is hydrogen. In some embodiments, R^{25b} is —CH₃.

In some embodiments, R^{25a} is hydrogen or unsubstituted C₁₋₆alkyl and R^{25b} is hydrogen or unsubstituted C₁₋₆alkyl. In some embodiments, R^{25a} is unsubstituted C₁₋₆alkyl and R^{25b} is hydrogen or unsubstituted C₁₋₆alkyl. In some embodiments, R^{25a} is hydrogen and R^{25b} is hydrogen or unsubstituted C₁₋₆alkyl. In some embodiments, R^{25a} is hydrogen or unsubstituted C₁₋₆alkyl and R^{25b} is hydrogen. In some embodiments, R^{25a} is hydrogen or unsubstituted C₁₋₆alkyl and R^{25b} is unsubstituted C₁₋₆alkyl. In some embodiments, R^{25a} is —CH₃ and R^{25b} is —CH₃. In some embodiments, R^{25a} is —CH₂CH₃ and R^{25b} is hydrogen. In some embodiments, R^{25a} is hydrogen and R^{25b} is hydrogen.

In some embodiments, R^{25a} and R^{25b}, taken together with the carbon atom to which they are attached, form a substituted or unsubstituted C₃₋₆carbocyclyl, or substituted or unsubstituted 3- to 10-membered heterocyclyl. In some embodiments, R^{25a} and R^{25b}, taken together with the carbon atom to which they are attached, form a substituted or unsubstituted C₃₋₆carbocyclyl. In some embodiments, R^{25a} and R^{25b} taken together with the carbon atom to which they are attached, form an unsubstituted C₃₋₆carbocyclyl. In some embodiments, R^{25a} and R^{25b} taken together with the carbon atom to which they are attached, form a cyclopropyl. In some embodiments, R^{25a} and R^{25b}, taken together with the carbon atom to which they are attached, form a substituted or unsubstituted 3- to 10-membered heterocyclyl.

q, r and s—as they Relate to Formulae (C-I)-(C-VI-B1-iia-2b)

In some embodiments, q is 0, 1 or 2. In some embodiments, q is 0. In some embodiments, q is 1. In some embodiments, q is 2.

In some embodiments, r is 1 or 2. In some embodiments, r is 1. In some embodiments, r is 2.

In some embodiments, s is 1 or 2. In some embodiments, s is 1. In some embodiments, s is 2.

In some embodiments, q is 0 and r is 1. In some embodiments, q is 2 and r is 1. In some embodiments, q, r and s are each independently 1.

In some embodiments, the compound of Formula (C-I) is any one of compounds C-1 to C-35 in Table C-1 or pharmaceutically acceptable salts thereof. In some embodiments, the compound of Formula (C-I) is any one of compounds C-1 to C-35 in Table C-1. In some embodiments, the compound of Formula (C-I) is a pharmaceutically acceptable salt of any one of Compounds C-1 to C-35 in Table C-1. In some embodiments, the compound of Formula (C-I) is any one of Compounds C-1, C-2, C-3, C-5, C-7, C-8, C-9, C-11, or pharmaceutically acceptable salts thereof. In some embodiments, the compound of Formula (C-I) is any one of Compounds C-1, C-2, C-3, C-5, C-7, C-8, C-9, or C-11. In some embodiments, the compound of Formula (C-I) is a pharmaceutically acceptable salt of any one of Compounds C-1, C-2, C-3, C-5, C-7, C-8, C-9, or C-11.

TABLE C-1

| Cmpd | Structure |
|---|---|
| C-1 | 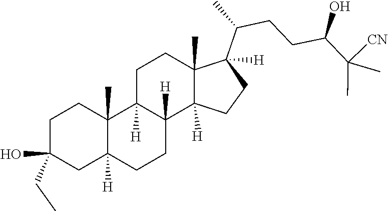 |
| C-2 | 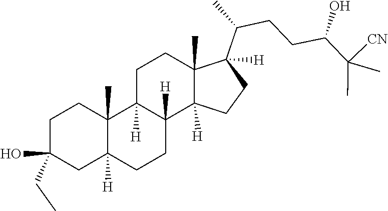 |
| C-3 | 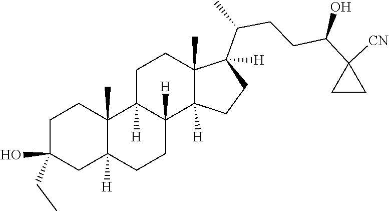 |
| C-4 | 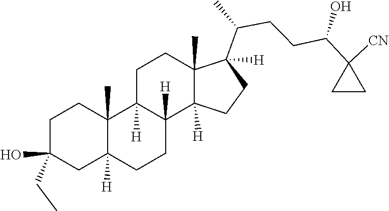 |
| C-5 | 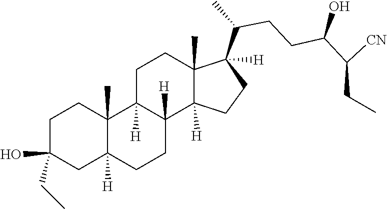 |
| C-6 | 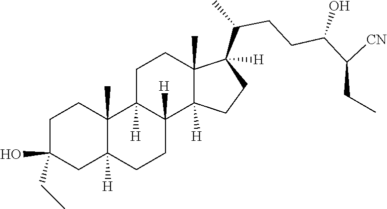 |
| C-7 | 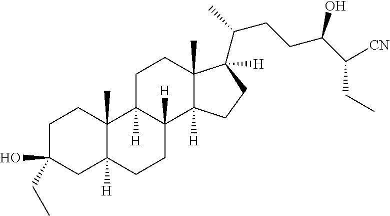 |
| C-8 | 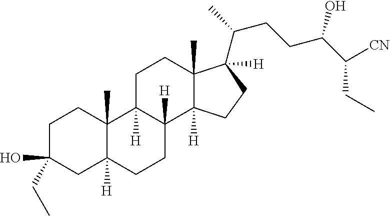 |
| C-9 | 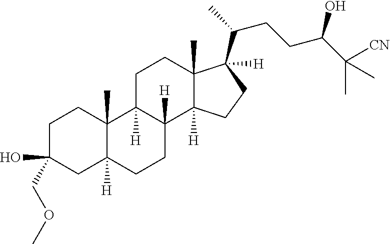 |
| C-10 | 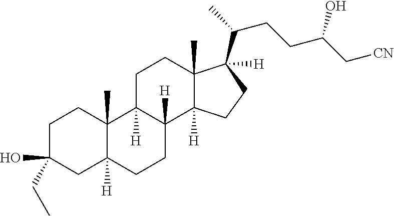 |
| C-11 | 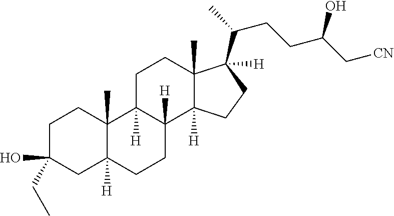 |
| C-12 | 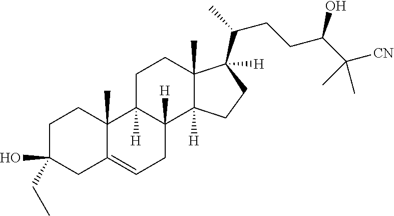 |
| C-13 | 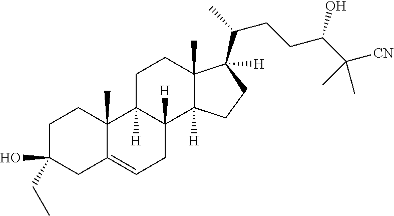 |
| C-14 | 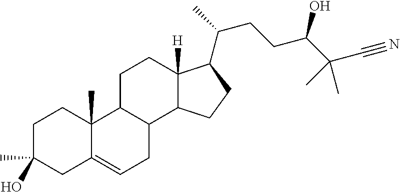 |
| C-15 | 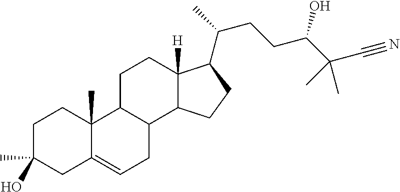 |
| C-16 | 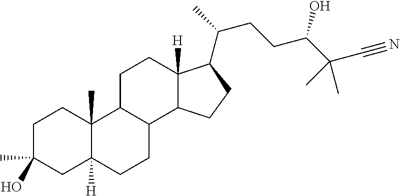 |
| C-17 | 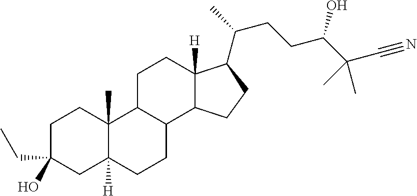 |
| C-18 | 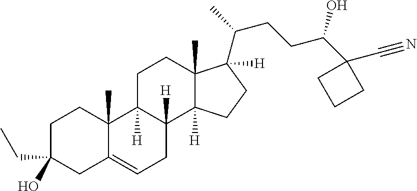 |
| C-19 | 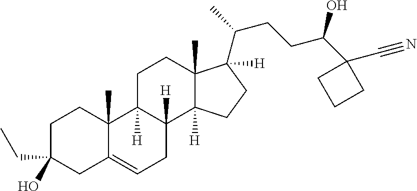 |
| C-20 | 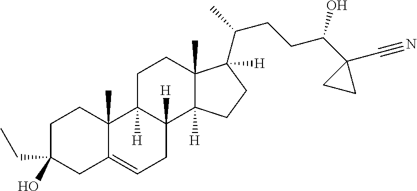 |
| C-21 | 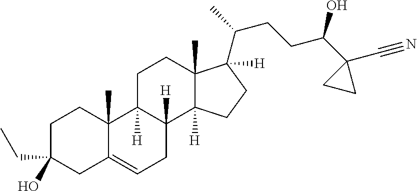 |
| C-22 | 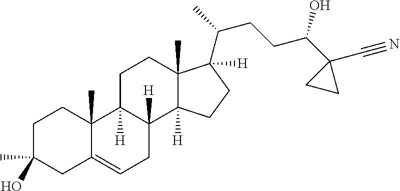 |
| C-23 | 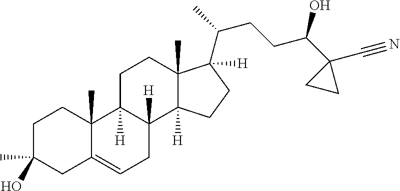 |
| C-24 | 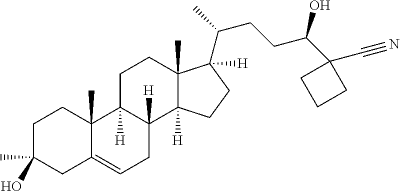 |
| C-25 | 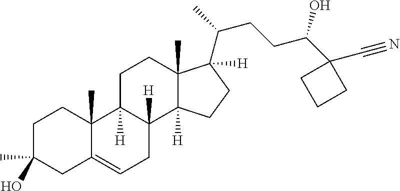 |
| C-26 | 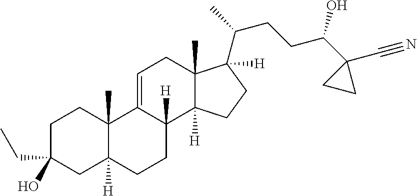 |
| C-27 | 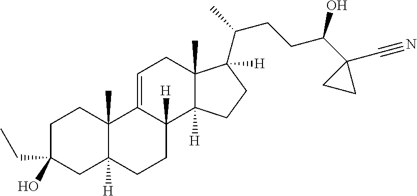 |
| C-28 | 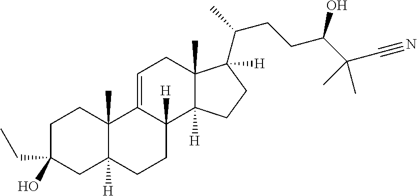 |
| C-29 | 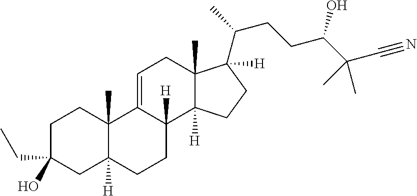 |
| C-30 | 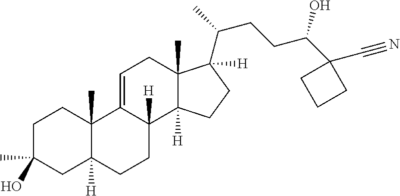 |
| C-31 | 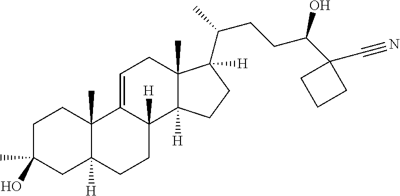 |
| C-32 | 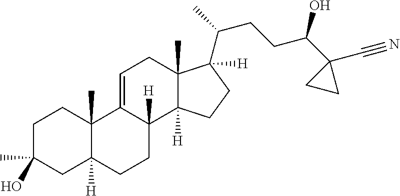 |
| C-33 | 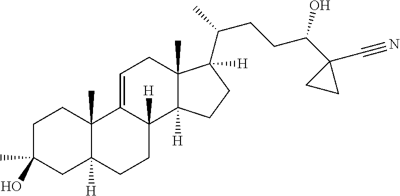 |
| C-34 | 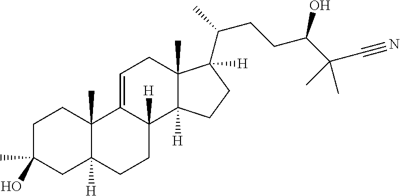 |
| C-35 | 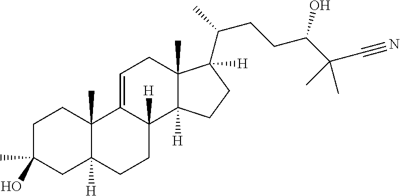 |

In one aspect, the disclosure provides a compound of Formula (D-I):

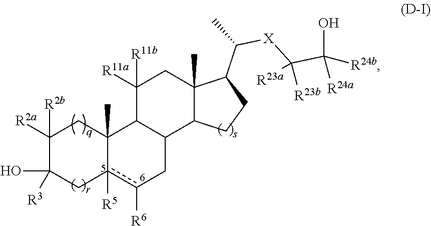

or a pharmaceutically acceptable salt thereof;
wherein:
   each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein R^{A2} is hydrogen or substituted or unsubstituted C₁₋₆alkyl;
   R³ is substituted or unsubstituted C₁₋₆alkyl, substituted or unsubstituted C₂₋₆alkenyl, or substituted or unsubstituted C₂₋₆alkynyl, or substituted or unsubstituted C₃₋₁₀carbocyclyl;
   R⁵ is absent, hydrogen, halogen, or substituted or unsubstituted C₁₋₆alkyl;
   R⁶ is hydrogen, substituted or unsubstituted C₁₋₆alkyl, or halogen;
   R^{11a} is hydrogen or —OR^{A11}, wherein R^{A11} is hydrogen or substituted or unsubstituted C₁₋₆alkyl, and R^{11b} is hydrogen; or R^{11a} and R^{11b} are joined to form an oxo (═O) group;
   X is —(C(R^{X})₂)ₙ— or —O—, wherein R^{X} is hydrogen or fluorine, or one R^{X} group and R^{23b} are joined to form a double bond;
   n is 1, 2 or 3;
   each of R^{23a} and R^{23b} is independently hydrogen or fluorine;
   R^{24a} is selected from the group consisting of substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl group; and
   R^{24b} is hydrogen or a substituted or unsubstituted C₁₋₆alkyl;
   or R^{24a} and R^{24b}, taken together with the carbon atom to which they are attached, form a substituted or unsubstituted C₃₋₆carbocyclyl, or substituted or unsubstituted 3- to 10-membered heterocyclyl;
   q is 0, 1 or 2;
   r is 1 or 2;
   s is 1, 2, 3 or 4;
   provided that a) q and r are not both 1, b) when q is 0, r is not 2, or c) when r is 2, q is not 0; and
   [symbol] represents a single or double bond;
   provided if the bond between C5 and C6 is a double bond, R⁵ is absent.

In some embodiments, the compound of Formula (D-I) is a compound of Formula (D-Ia) or (D-Ib):

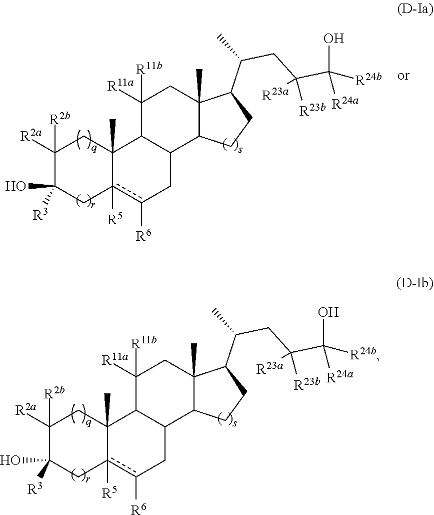

or a pharmaceutically acceptable salt thereof.

In some embodiments, the compound of Formula (D-Ia) is a compound of Formula (D-Ia-1):

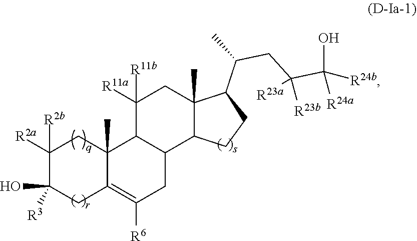

or a pharmaceutically acceptable salt thereof.

In some embodiments, the compound of Formula (D-Ia-1) is a compound of Formula (D-Ia-1i):

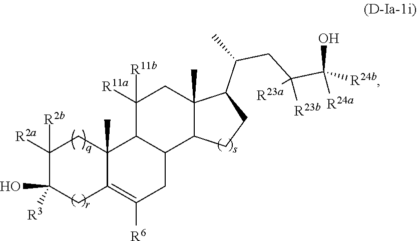

or a pharmaceutically acceptable salt thereof.

In some embodiments, the compound of Formula (D-Ia-1) is a compound of Formula (D-Ia-1ii):

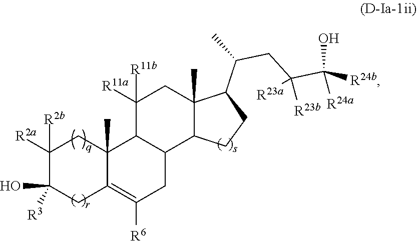

or a pharmaceutically acceptable salt thereof.

In some embodiments, the compound of Formula (D-Ia) is a compound of Formula (D-Ia-2):

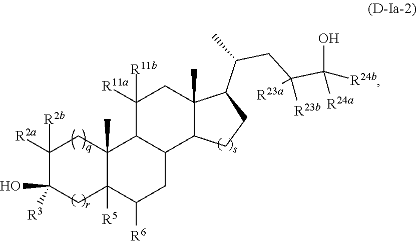

or a pharmaceutically acceptable salt thereof.

In some embodiments, the compound of Formula (D-Ia-2) is a compound of Formula (D-Ia-2i):

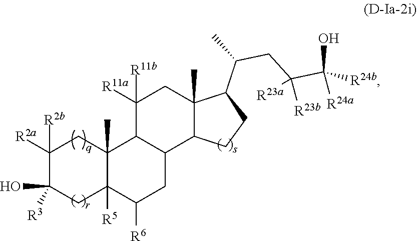

or a pharmaceutically acceptable salt thereof.

In some embodiments, the compound of Formula (D-Ia-2) is a compound of Formula (D-Ia-2ii):

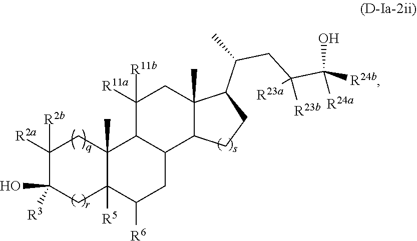

or a pharmaceutically acceptable salt thereof.

In some embodiments, the compound of Formula (D-Ib) is a compound of Formula (D-Ib-1):

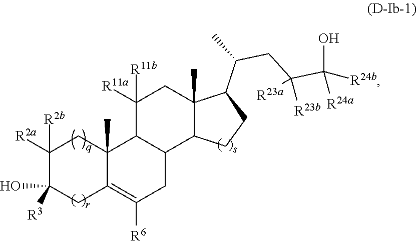

or a pharmaceutically acceptable salt thereof.

In some embodiments, the compound of Formula (D-Ib-1) is a compound of Formula (D-Ib-1i):

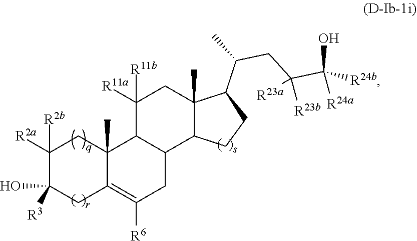

or a pharmaceutically acceptable salt thereof.

In some embodiments, the compound of Formula (D-Ib-1) is a compound of Formula (D-Ib-1ii):

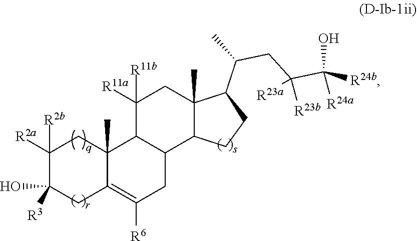

or a pharmaceutically acceptable salt thereof.

In some embodiments, the compound of Formula (D-Ib) is a compound of Formula (D-Ib-2):

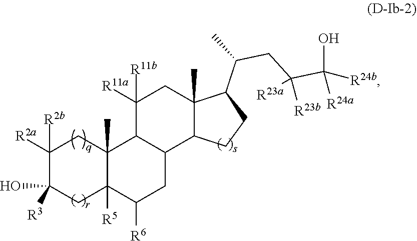

or a pharmaceutically acceptable salt thereof.

In some embodiments, the compound of Formula (D-Ib-2) is a compound of Formula (D-Ib-2i):

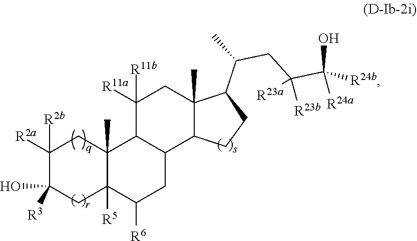

or a pharmaceutically acceptable salt thereof.

In some embodiments, the compound of Formula (D-Ib-2) is a compound of Formula (D-Ib-2ii):

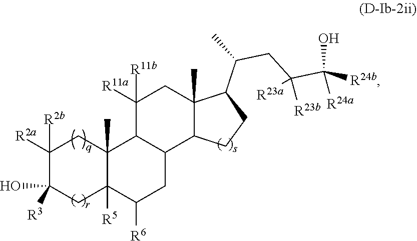

or a pharmaceutically acceptable salt thereof.

In some embodiments, the compound of Formula (D-I) is a compound of Formula (D-II):

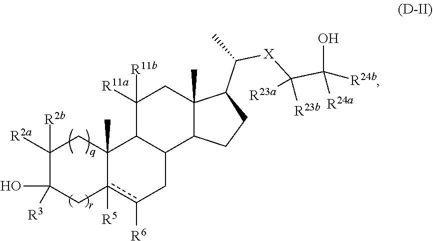

or a pharmaceutically acceptable salt thereof.

In some embodiments, the compound of Formula (D-II) is a compound of Formula (D-IIa) or (D-IIb):

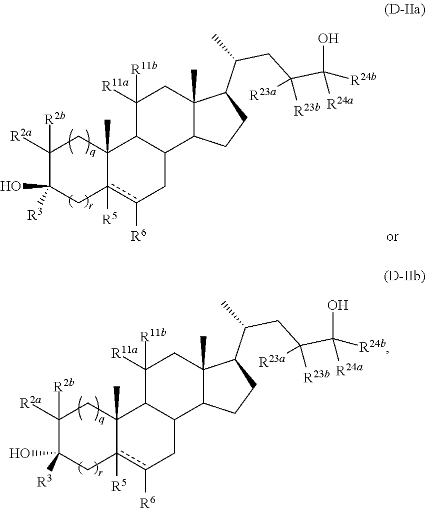

or a pharmaceutically acceptable salt thereof.

In some embodiments, the compound of Formula (D-IIa) is a compound of Formula (D-Ia-1):

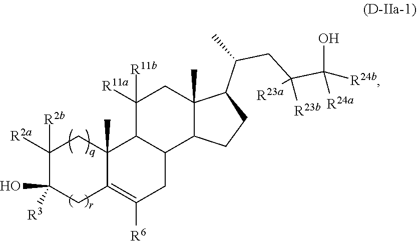

or a pharmaceutically acceptable salt thereof.

In some embodiments, the compound of Formula (D-IIa-1) is a compound of Formula (D-Ia-1i):

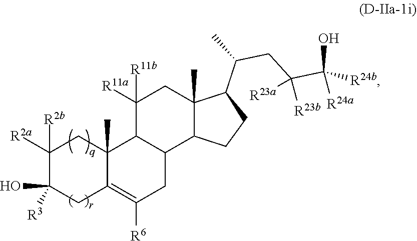

or a pharmaceutically acceptable salt thereof.

In some embodiments, the compound of Formula (D-IIa-1) is a compound of Formula (D-IIa-1ii):

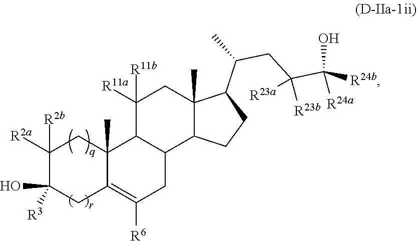

or a pharmaceutically acceptable salt thereof.

In some embodiments, the compound of Formula (D-IIa) is a compound of Formula (D-IIa-2):

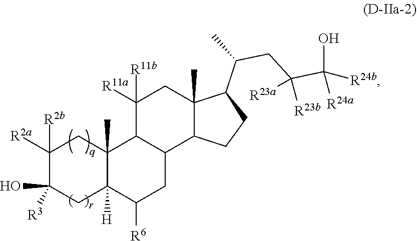

or a pharmaceutically acceptable salt thereof.

In some embodiments, the compound of Formula (D-IIa-2) is a compound of Formula (D-IIa-2i):

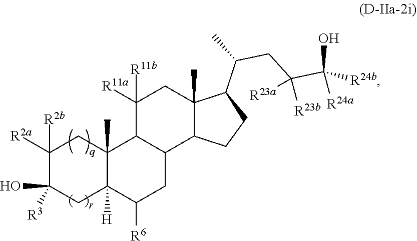

or a pharmaceutically acceptable salt thereof.

In some embodiments, the compound of Formula (D-Ia-2) is a compound of Formula (D-IIa-2ii):

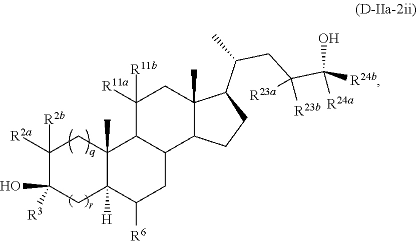

or a pharmaceutically acceptable salt thereof.

In some embodiments, the compound of Formula (D-IIb) is a compound of Formula (D-IIb-1):

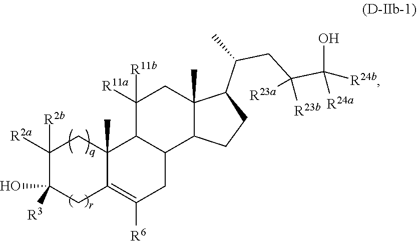

or a pharmaceutically acceptable salt thereof.

In some embodiments, the compound of Formula (D-IIb-1) is a compound of Formula (D-IIb-1i):

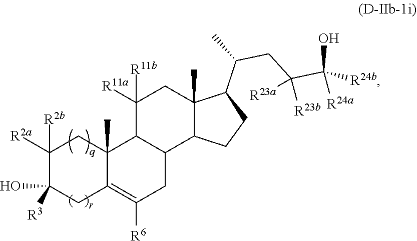

or a pharmaceutically acceptable salt thereof.

In some embodiments, the compound of Formula (D-IIb-1) is a compound of Formula (D-IIb-1ii):

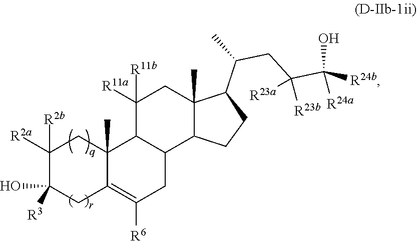

or a pharmaceutically acceptable salt thereof.

In some embodiments, the compound of Formula (D-LIb) is a compound of Formula (D-IIb-2):

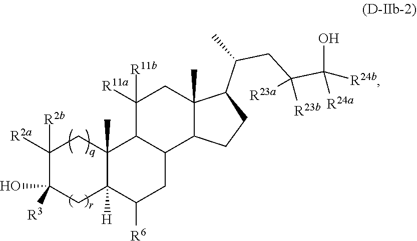

or a pharmaceutically acceptable salt thereof.

In some embodiments, the compound of Formula (D-IIb-2) is a compound of Formula (D-IIb-2i):

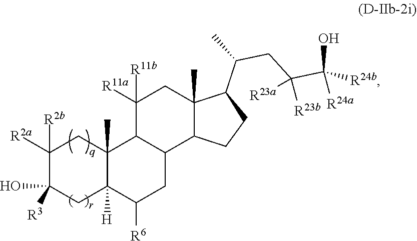

or a pharmaceutically acceptable salt thereof.

In some embodiments, the compound of Formula (D-IIb-2) is a compound of Formula (D-IIb-2ii):

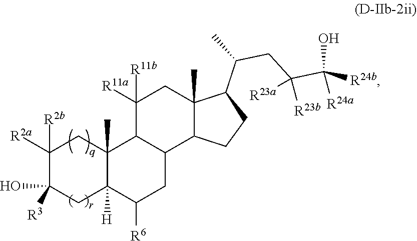

or a pharmaceutically acceptable salt thereof.

In some embodiments, the compound of Formula (D-I) is a compound of Formula (D-III):

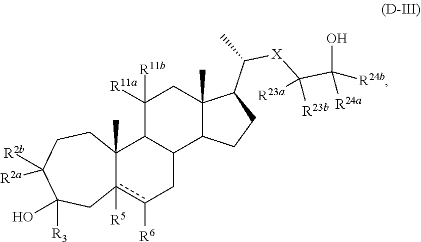

or a pharmaceutically acceptable salt thereof.

In some embodiments, the compound of Formula (D-III) is a compound of Formula (D-IIIa) or Formula (D-IIIb):

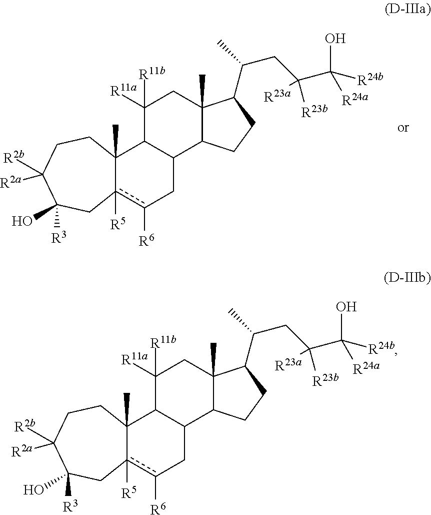

or a pharmaceutically acceptable salt thereof.

In some embodiments, the compound of Formula (D-IIIa) is a compound of Formula (D-IIIa-1):

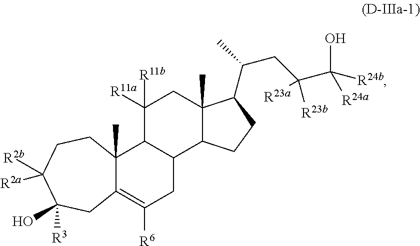

or a pharmaceutically acceptable salt thereof.

In some embodiments, the compound of Formula (D-IIIa-1) is a compound of Formula (D-IIIa-1i):

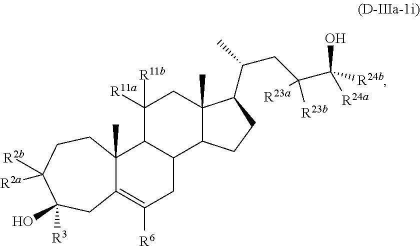

or a pharmaceutically acceptable salt thereof.

In some embodiments, the compound of Formula (D-IIIa-1) is a compound of Formula (D-IIIa-1ii):

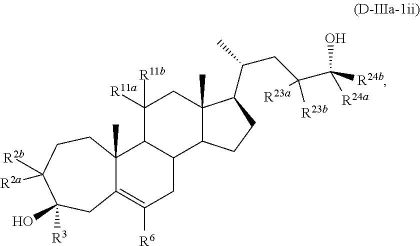

or a pharmaceutically acceptable salt thereof.

In some embodiments, the compound of Formula (D-IIIa) is a compound of Formula (D-IIIa-2):

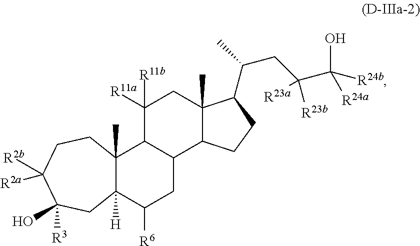

or a pharmaceutically acceptable salt thereof.

In some embodiments, the compound of Formula (D-IIIa-2) is a compound of Formula (D-IIIa-2i):

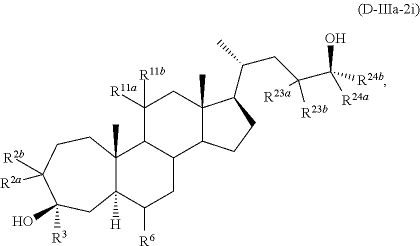

or a pharmaceutically acceptable salt thereof.

In some embodiments, the compound of Formula (D-IIIa-2) is a compound of Formula (D-IIIa-2ii):

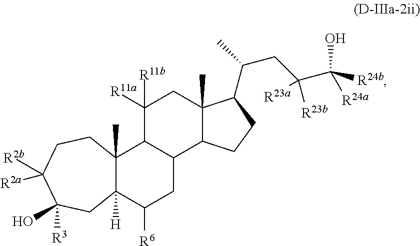

or a pharmaceutically acceptable salt thereof.

In some embodiments, the compound of Formula (D-IIIb) is a compound of Formula (D-IIIb-1):

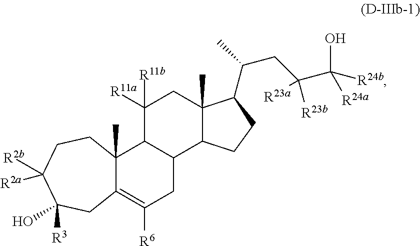

or a pharmaceutically acceptable salt thereof.

In some embodiments, the compound of Formula (D-IIIb-1) is a compound of Formula (D-IIIb-1i):

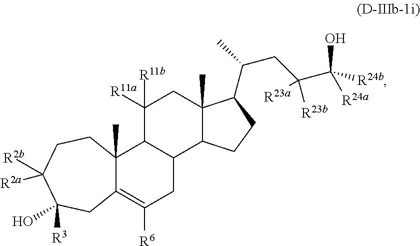

or a pharmaceutically acceptable salt thereof.

In some embodiments, the compound of Formula (D-IIIb-1) is a compound of Formula (D-IIIb-1ii):

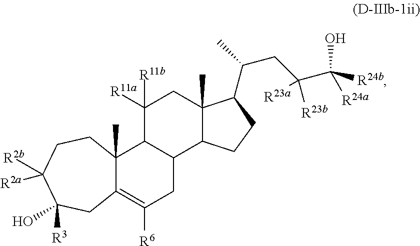

or a pharmaceutically acceptable salt thereof.

In some embodiments, the compound of Formula (D-IIIb) is a compound of Formula (D-IIIb-2):

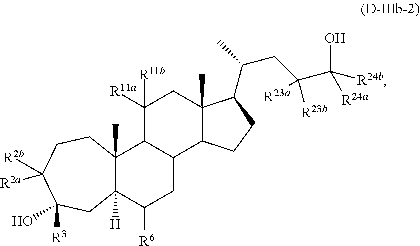

or a pharmaceutically acceptable salt thereof.

In some embodiments, the compound of Formula (D-IIIb-2) is a compound of Formula (D-IIIb-2i):

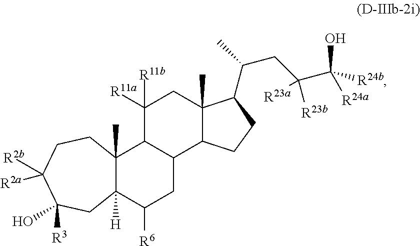

or a pharmaceutically acceptable salt thereof.

In some embodiments, the compound of Formula (D-IIIb-2) is a compound of Formula (D-IIIb-2ii):

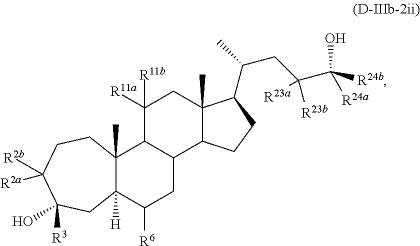

or a pharmaceutically acceptable salt thereof.

In some embodiments, the compound of Formula (D-I) is a compound of Formula (D-IV):

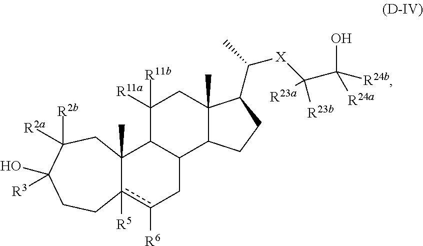

or a pharmaceutically acceptable salt thereof.

In some embodiments, the compound of Formula (D-IV) is a compound of Formula (D-IVa) or Formula (D-IVb):

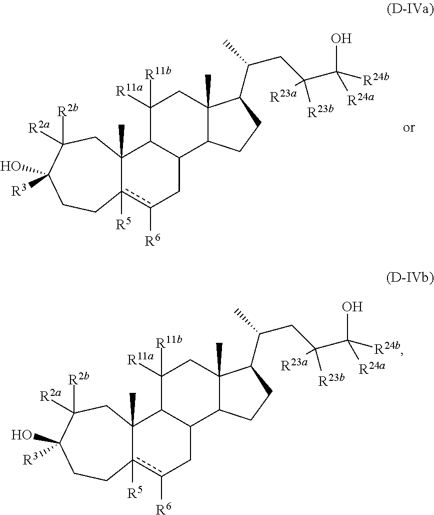

or a pharmaceutically acceptable salt thereof.

In some embodiments, the compound of Formula (D-IVa) is a compound of Formula (D-IVa-1):

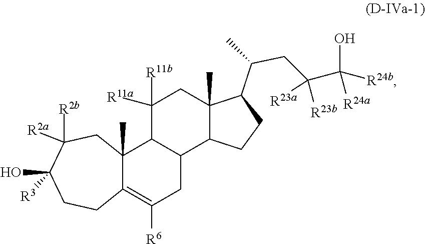

or a pharmaceutically acceptable salt thereof.

In some embodiments, the compound of Formula (D-IVa-1) is a compound of Formula (D-IVa-1i):

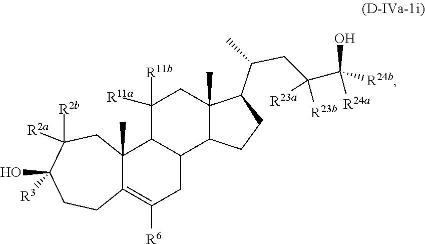

or a pharmaceutically acceptable salt thereof.

In some embodiments, the compound of Formula (D-IVa-1) is a compound of Formula (D-IVa-1ii):

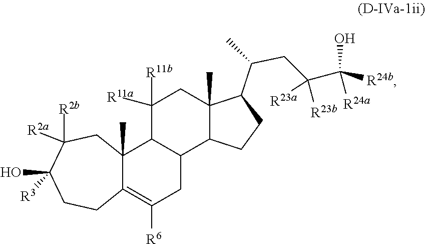

or a pharmaceutically acceptable salt thereof.

In some embodiments, the compound of Formula (D-IVa) is a compound of Formula (D-IVa-2):

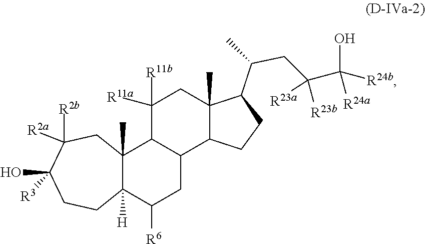

or a pharmaceutically acceptable salt thereof.

In some embodiments, the compound of Formula (D-IVa-2) is a compound of Formula (D-IVa-2i):

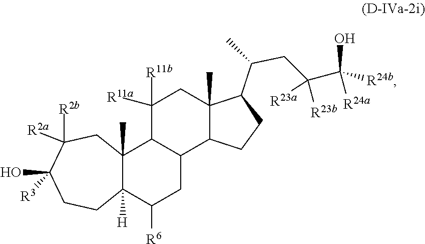

or a pharmaceutically acceptable salt thereof.

In some embodiments, the compound of Formula (D-IVa-2) is a compound of Formula (D-IVa-2ii):

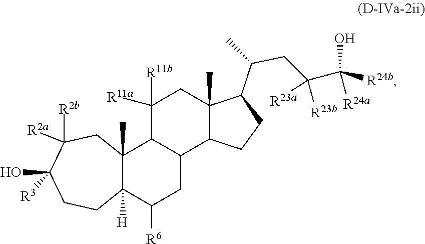

or a pharmaceutically acceptable salt thereof.

In some embodiments, the compound of Formula (D-IVb) is a compound of Formula (D-IVb-1):

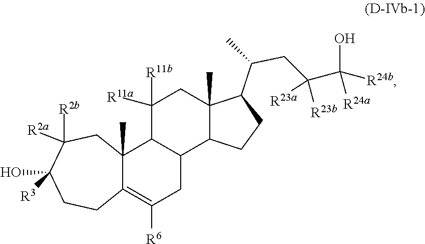

or a pharmaceutically acceptable salt thereof.

In some embodiments, the compound of Formula (D-IVb-1) is a compound of Formula (D-IVb-1i):

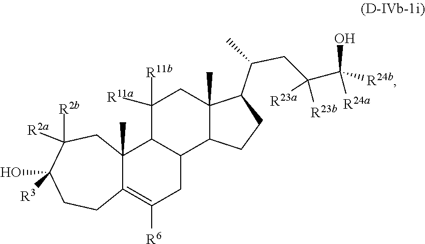

or a pharmaceutically acceptable salt thereof.

In some embodiments, the compound of Formula (D-IVb-1) is a compound of Formula (D-IVb-1ii):

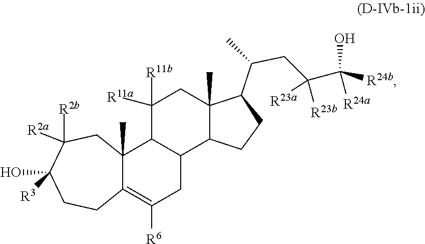

or a pharmaceutically acceptable salt thereof.

In some embodiments, the compound of Formula (D-IVb) is a compound of Formula (D-IVb-2):

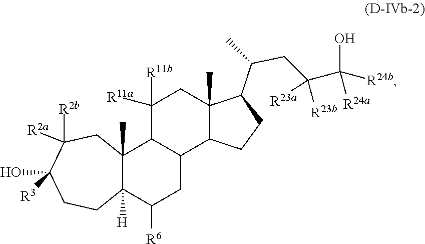

or a pharmaceutically acceptable salt thereof.

In some embodiments, the compound of Formula (D-IVb-2) is a compound of Formula (D-IVb-2i):

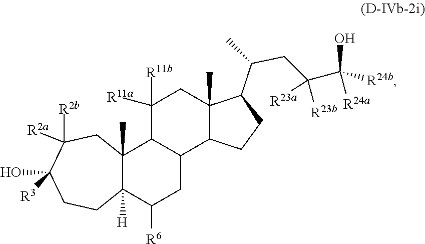

or a pharmaceutically acceptable salt thereof.

In some embodiments, the compound of Formula (D-IVb-2) is a compound of Formula (D-IVb-2ii):

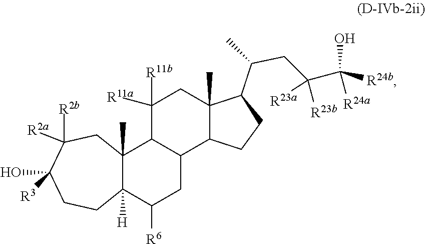

or a pharmaceutically acceptable salt thereof.
   Groups R^{2a} and R^{2b}—as they Relate to Formulae (D-I)-(D-IVb-2ii)

In certain embodiments, each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein R^{A2} is hydrogen or substituted or unsubstituted C₁₋₆alkyl. In certain embodiments, each of R^{2a} and R^{2b} is hydrogen. In certain embodiments, each of R^{2a} and R^{2b} is independently halogen, e.g., fluoro, chloro, bromo, or iodo. In certain embodiments, each of R^{2a} and R^{2b} is independently fluoro or chloro. In certain embodiments, each of R^{2a} and R^{2b} is substituted or unsubstituted C₁₋₆alkyl, e.g., substituted or unsubstituted C₁₋₂ alkyl, substituted or unsubstituted C₂₋₃ alkyl, substituted or unsubstituted C₃₋₄ alkyl, substituted or unsubstituted C₄₋₅alkyl, or substituted or unsubstituted C₅₋₆ alkyl. In certain embodiments, each of R^{2a} and R^{2b} is independently —CH₃, —CH₂CH₃, —CH₂CH₂CH₃, or cyclopropyl. In certain embodiments, each of R^{2a} and R^{2b} is —OR^{A2}. In certain embodiments, R^{A2} is hydrogen. In certain embodiments, R^{A2} is substituted or unsubstituted alkyl, e.g., substituted or unsubstituted C₁₋₆alkyl, substituted or unsubstituted C₁₋₂ alkyl, substituted or unsubstituted C₂₋₃ alkyl, substituted or unsubstituted C₃₋₄ alkyl, substituted or unsubstituted C₄₋₅ alkyl, or substituted or unsubstituted C₅₋₆ alkyl. In certain embodiments, R^{A2} is hydrogen, —CH₃, —CH₂CH₃, or —CH₂CH₂CH₃, i.e., to provide a group of R^{2a} or R^{2b} each independently of formula-OH, —OCH₃, —OCH₂CH₃, or —OCH₂CH₂CH₃. In certain embodiments, each of R^{2a} and R^{2b} is a non-hydrogen substituent. In certain embodiments, one of R^{2a} and R^{2b} is a non-hydrogen substituent in the alpha configuration. In certain embodiments, one of R^{2a} and R^{2b} is a non-hydrogen substituent in the beta configuration.

### Group R³—as it Relates to Formulae (D-I)-(D-IVb-2ii)

In certain embodiments, R³ is substituted or unsubstituted aliphatic, i.e., substituted or unsubstituted alkyl, substituted or unsubstituted alkenyl, substituted or unsubstituted alkynyl, or substituted or unsubstituted carbocyclyl.

In certain embodiments, R³ is substituted or unsubstituted C₁₋₆alkyl, substituted or unsubstituted C₂₋₆alkenyl, substituted or unsubstituted C₂₋₆alkynyl, or substituted or unsubstituted C₃₋₁₀carbocyclyl.

In certain embodiments, R³ is substituted or unsubstituted alkyl, e.g., substituted or unsubstituted C₁₋₆alkyl, substituted or unsubstituted C₁₋₂ alkyl, substituted or unsubstituted C₂₋₃ alkyl, substituted or unsubstituted C₃₋₄ alkyl, substituted or unsubstituted C₄₋₅ alkyl, or substituted or unsubstituted C₅₋₆ alkyl. Exemplary R³ C₁₋₆alkyl groups include, but are not limited to, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), n-hexyl (C₆), C₁₋₆ alkyl substituted with 1, 2, 3, 4, 5, 6, 7, 8, 9, 10, or more fluoro groups (e.g., —CF₃, —CH₂F, —CHF₂, difluoroethyl, and 2,2,2-trifluoro-1,1-dimethyl-ethyl), C₁₋₆alkyl substituted with 1, 2, 3, 4, 5, 6, 7, 8, 9, 10, or more chloro groups (e.g., —CH₂Cl, —CHCl₂), and C₁₋₆alkyl substituted with 1, 2, 3, 4, 5, 6, 7, 8, 9, 10, or more alkoxy groups (e.g., —CH₂OCH₃, —CH₂OCH₂CH₃, —CH₂O-cyclopropyl). In certain embodiments, R³ is substituted alkyl, e.g., R³ is haloalkyl, alkoxyalkyl, or aminoalkyl. In certain embodiments, R³ is Me, Et, n-Pr, n-Bu, i-Bu, fluoromethyl, chloromethyl, difluoromethyl, trifluoromethyl, trifluoroethyl, difluoroethyl, 2,2,2-trifluoro-1,1-dimethyl-ethyl, methoxymethyl, methoxyethyl, or ethoxymethyl.

In certain embodiments, R³ is substituted or unsubstituted C₁₋₆alkyl. In certain embodiments, R³ is unsubstituted C₁₋₆alkyl. In some embodiments, R³ is selected from the group consisting of CH₃, —CH₂CH₃, or —CH₂CH₂CH₃. In some embodiments, R³ is ethyl.

In certain embodiments, R³ is substituted C₁₋₆alkyl. In certain embodiments, R³ is alkyl substituted with one or more fluorine atoms, e.g., R³ is —CH₂F, —CHF₂, or —CF₃. In certain embodiments, R³ is alkyl substituted with one or more —OR^{A3} groups, wherein R^{A3} is hydrogen or substituted or unsubstituted alkyl. In certain embodiments, R³ is —CH₂OR^{A3}, e.g., wherein RA₃ is hydrogen, —CH₃, —CH₂CH₃, or —CH₂CH₂CH₃.

In certain embodiments, R³ is —CH₃, —CH₂CH₃, —CH₂CH₂CH₃, or —CH₂OCH₃. In certain embodiments, R³ is —CH₂CH₃. In certain embodiments, R³ is —CH₂OCH₃.

In certain embodiments, R³ is substituted or unsubstituted alkenyl, e.g., substituted or unsubstituted C₂₋₆alkenyl, substituted or unsubstituted C₂₋₃alkenyl, substituted or unsubstituted C₃₋₄ alkenyl, substituted or unsubstituted C₄₋₅alkenyl, or substituted or unsubstituted C₅₋₆ alkenyl. In certain embodiments, R³ is ethenyl (C₂), propenyl (C₃), or butenyl (C₄), unsubstituted or substituted with one or more substituents selected from the group consisting of alkyl, halo, haloalkyl, alkoxyalkyl, or hydroxyl. In certain embodiments, R³ is ethenyl, propenyl, or butenyl, unsubstituted or substituted with alkyl, halo, haloalkyl, alkoxyalkyl, or hydroxy. In certain embodiments, R³ is ethenyl.

In certain embodiments, R³ is substituted or unsubstituted alkynyl, e.g., substituted or unsubstituted C₂₋₆alkynyl, substituted or unsubstituted C₂₋₃ alkynyl, substituted or unsubstituted C₃₋₄ alkynyl, substituted or unsubstituted C₄₋₅alkynyl, or substituted or unsubstituted C₅₋₆ alkynyl. Exemplary substituted or unsubstituted R³ alkynyl groups include, but are not limited to, ethynyl, propynyl, or butynyl, unsubstituted or substituted with alkyl, halo, haloalkyl (e.g., CF₃), alkoxyalkyl, cycloalkyl (e.g., cyclopropyl or cyclobutyl), or hydroxyl. In certain embodiments, R³ is selected from the group consisting of trifluoroethynyl, cyclopropylethynyl, cyclobutylethynyl, and propynyl, fluoropropynyl, and chloroethynyl. In certain embodiments, R³ is ethynyl (C₂), propynyl (C₃), or butynyl (C₄), unsubstituted or substituted with one or more substituents selected from the group consisting of substituted or unsubstituted aryl, substituted or unsubstituted heteroaryl, substituted or unsubstituted carbocyclyl, and substituted or unsubstituted heterocyclyl. In certain embodiments, R³ is ethynyl (C₂), propynyl (C₃), or butynyl (C₄) substituted with substituted phenyl. In certain embodiments, the phenyl substituent is further substituted with one or more substituents selected from the group consisting of halo, alkyl, trifluoroalkyl, alkoxy, acyl, amino or amido. In certain embodiments, R³ is ethynyl (C₂), propynyl (C₃), or butynyl (C₄) substituted with substituted or unsubstituted pyrrolyl, imidazolyl, pyrazolyl, oxazoyl, thiazolyl, isoxazoyl, 1,2,3-triazolyl, 1,2,4-triazolyl, oxadiazolyl, thiadiazolyl, or tetrazolyl.

In certain embodiments, R³ is ethynyl, propynyl, or butynyl, unsubstituted or substituted with alkyl, halo, haloalkyl, alkoxyalkyl, or hydroxyl. In certain embodiments, R³ is ethynyl or propynyl, substituted with substituted or unsubstituted aryl. In certain embodiments, R³ is ethynyl or propynyl, substituted with phenyl unsubstituted or substituted with halo, alkyl, alkoxy, haloalkyl, trihaloalkyl, or acyl. In certain embodiments, R³ is ethynyl or propynyl, substituted with substituted or unsubstituted carbocyclyl. In certain embodiments, R³ is ethynyl or propynyl, substituted with substituted or unsubstituted cyclopropyl, cyclobutyl, cyclopentyl, or cyclohexyl. In certain embodiments, R³ is ethynyl or propynyl, substituted with substituted or unsubstituted heteroaryl. In certain embodiments, R³ is ethynyl or propynyl, substituted with substituted or unsubstituted pyridinyl, or pyrimidinyl. In certain embodiments, R³ is ethynyl or propynyl, substituted with substituted or unsubstituted pyrrolyl, imidazolyl, pyrazolyl, oxazoyl, thiazolyl, isoxazoyl, 1,2,3-triazolyl, 1,2,4-triazolyl, oxadiazolyl, thiadiazolyl, tetrazolyl. In certain embodiments, R³ is ethynyl or propynyl, substituted with substituted or unsubstituted heterocyclyl. In certain embodiments, R³ is ethynyl or propynyl, substituted with substituted or unsubstituted pyrrolidinyl, piperidinyl, piperazinyl, or mopholinyl. In certain embodiments, R³ is propynyl or butynyl, substituted with hydroxyl or alkoxy. In certain embodiments, R³ is propynyl or butynyl, substituted with methoxy or ethoxy. In certain embodiments, R³ is ethynyl or propynyl, substituted with chloro. In certain embodiments, R³ is ethynyl or propynyl, substituted with trifluoromethyl.

In certain embodiments, R³ is substituted or unsubstituted carbocyclyl, e.g., substituted or unsubstituted C₃₋₁₀carbocyclyl, substituted or unsubstituted C₃₋₆carbocyclyl, substituted or unsubstituted C₃₋₄ carbocyclyl, substituted or unsubstituted C₄₋₅ carbocyclyl, or substituted or unsubstituted C₅₋₆ carbocyclyl. In certain embodiments, R³ is substituted or unsubstituted cyclopropyl or substituted or unsubstituted cyclobutyl.

In certain embodiments, R³ is in the alpha configuration. In certain embodiments, R³ is in the beta configuration.

Groups R⁵ and R⁶—as they Relate to Formulae (D-1)-(D-IVb-2ii)

In some embodiments, R⁵ is in the alpha or beta configuration. In certain embodiments, R⁵ is in the alpha configuration. In certain embodiments, R⁵ is in the beta configuration. In some embodiments, when the bond between C5 and C6 is a double bond, R⁵ is absent. In some embodiments, the bond between C5 and C6 is a single bond and R⁵ is in the alpha configuration. In some embodiments, the bond between C5 and C6 is a single bond and R⁵ is hydrogen in the alpha configuration.

In certain embodiments, R⁵ is absent, hydrogen, halogen, or substituted or unsubstituted C₁₋₆alkyl. In some embodiments, R⁵ is halogen. In some embodiments, R⁵ is substituted C₁₋₆alkyl. In certain embodiments, R⁵ is substituted or unsubstituted alkyl, e.g., substituted or unsubstituted C₁₋₆alkyl, substituted or unsubstituted C₁₋₂ alkyl, substituted or unsubstituted C₂₋₃ alkyl, substituted or unsubstituted C₃₋₄ alkyl, substituted or unsubstituted C₄₋₅alkyl, or substituted or unsubstituted C₅₋₆ alkyl. In some embodiments, R⁵ is substituted or unsubstituted C₁₋₆alkyl. In some embodiments, R⁵ is unsubstituted C₁₋₆alkyl. In some embodiments, R⁵ is unsubstituted C₁ alkyl. In some embodiments, R⁵ is —CH₃. In some embodiments, R⁵ is hydrogen.

In certain embodiments, R⁶ is hydrogen, halogen, or substituted or unsubstituted C₁₋₆alkyl. In certain embodiments, R⁶ is halogen, e.g., fluoro. In certain embodiments, R⁶ is substituted or unsubstituted alkyl, e.g., substituted or unsubstituted C₁₋₆alkyl, substituted or unsubstituted C₁₋₂ alkyl, substituted or unsubstituted C₂₋₃ alkyl, substituted or unsubstituted C₃₋₄alkyl, substituted or unsubstituted C₄₋₅ alkyl, or substituted or unsubstituted C₅₋₆ alkyl. In certain embodiments, R⁶ is C₁ alkyl, e.g., —CH₃ or —CF₃. In certain embodiments, R⁶ is hydrogen, —CH₃, or —F. In certain embodiments, wherein [symbol] represents a single bond, R⁶ is a non-hydrogen substituent in the alpha configuration. In certain embodiments, wherein [symbol] represents a single bond, R⁶ is a non-hydrogen substituent in the beta configuration.

Groups R^{11a} and R^{11b}—as they Relate to Formulae (D)-I)-(D)-IVb-2ii)

In certain embodiments, R^{11a} is hydrogen or —OR^{A11}, wherein R^{A11} is hydrogen or substituted or unsubstituted C₁₋₆alkyl, and R^{11b} is hydrogen; or R^{11a} and R^{11b} are joined to form an oxo (═O) group.

In certain embodiments, R^{11a} and R^{11b} are joined to form an oxo (═O) group.

In certain embodiments, R^{11a} is —OR^{A11} and R^{11b} is hydrogen. In certain embodiments, R^{11a} is —OR^{A11} in the alpha or beta configuration. In certain embodiments, R^{11a} is —OR^{A11} in the alpha configuration. In certain embodiments, R^{11a} is —OR^{A11} in the beta configuration. In certain embodiments, R^{A11} is hydrogen. In certain embodiments, R^{A11} is substituted or unsubstituted alkyl, e.g., substituted or unsubstituted C₁₋₆alkyl, substituted or unsubstituted C₁₋₂ alkyl, substituted or unsubstituted C₂₋₃ alkyl, substituted or unsubstituted C₃₋₄alkyl, substituted or unsubstituted C₄₋₅ alkyl, or substituted or unsubstituted C₅₋₆ alkyl. In certain embodiments, R^{A11} is hydrogen, —CH₃, —CH₂CH₃, or —CH₂CH₂CH₃, i.e., to provide a group R^{11a} of formula-OH, —OCH₃, —OCH₂CH₃, or —OCH₂CH₂CH₃.

In some embodiments, each of R^{11a} and R^{11b} is hydrogen.

Groups X, n, R^{23a}, R^{23b}, R^{24a}, and R^{24b}—as they Relate to Formulae (D-I)-(D-IVb-2ii)

In certain embodiments X is —(C(R^{X})₂)ₙ— or —O—, wherein R^{X} is hydrogen or fluorine, or one R^{X} group and R^{23b} are joined to form a double bond.

In certain embodiments, X is —O—. In certain embodiments, X is —CH₂—. In certain embodiments, X is —CF₂—.

In certain embodiments, n is selected from 1, 2 or 3. In some embodiments, n is 1. In some embodiments, n is 2. In some embodiments, n is 3.

In some embodiments, X is —(C(R^{X})₂—, wherein R^{X} is hydrogen or fluorine. In some embodiments, X is —CH₂—. In some embodiments, X is —CF₂—.

In some embodiments, X is —(C(R^{X})₂)₂—, wherein R^{X} is hydrogen or fluorine. In some embodiments, X is —(CH₂)₂—. In some embodiments, X is —(CF₂)₂—.

In some embodiments, each of R^{23a} and R^{23b} is independently hydrogen or fluorine. In some embodiments, each of R^{23a} and R^{23b} is hydrogen. In some embodiments, at least one of R^{23a} and R^{23b} is fluorine. In some embodiments, each of R^{23a} and R^{23b} is fluorine. In certain embodiments, at least one of R^{23a} and R^{23b} is hydrogen. In certain embodiments, R^{X} and R^{23b} are joined to form a double bond, e.g., cis or trans double bond.

In some embodiments, R^{24a} is selected from the group consisting of substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl; and R^{24b} is hydrogen or a substituted or unsubstituted C₁₋₆alkyl; or R^{24a} and R^{24b}, taken together with the carbon atom to which they are attached, form a substituted or unsubstituted C₃₋₆carbocyclyl, or a substituted or unsubstituted 3- to 10-membered heterocyclyl.

In some embodiments, R^{24a} is selected from the group consisting of substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl; and R^{24b} is hydrogen or a substituted or unsubstituted C₁₋₆alkyl; or R^{24a} and R^{24b}, taken together with the carbon atom to which they are attached, form a substituted or unsubstituted C₃₋₆carbocyclyl, or a substituted or unsubstituted 3- to 10-membered heterocyclyl.

In some embodiments, R^{24a} is a non-hydrogen group selected from substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, or substituted or unsubstituted 5- to 10-membered heteroaryl. In some embodiments, R^{24a} is a substituted or unsubstituted C₂₋₄alkyl, substituted or unsubstituted C₂₋₃alkenyl, substituted or unsubstituted C₂₋₃ alkynyl, or substituted or unsubstituted C₃carbocyclyl. In some embodiments, R^{24a} is a substituted or unsubstituted C₁₋₄alkyl, substituted or unsubstituted C₂₋₃alkenyl, substituted or unsubstituted C₂₋₃ alkynyl, or substituted or unsubstituted C₃carbocyclyl. In some embodiments, R^{24a} is a non-hydrogen group substituted with fluorine. In some embodiments, R^{24a} is a non-hydrogen group substituted with one or more —OR^{A24} groups, wherein R^{A24} is hydrogen or substituted or unsubstituted C₁₋₆alkyl.

In some embodiments, R^{24a} is a substituted or unsubstituted C₁₋₆alkyl. In some embodiments, R^{24a} is unsubstituted C₁₋₆alkyl. In some embodiments, R^{24a} is substituted C₁₋₆ alkyl. In some embodiments, R^{24a} is —CH₃, —CH₂CH₃, —CH(CH₃)₂, or —CF₃. In some embodiments, R^{24a} is —CH₃, —CH(CH₃)₂, or —CF₃. In some embodiments, R^{24a} is —CH₃. In some embodiments, R^{24a} is —CH(CH₃)₂. In some embodiments, R^{24a} is —CF₃. In some embodiments, R^{24a} is —CH₂OR^{A24}, —CH₂CH₂OR^{A24}, or —CH₂CH₂CH₂OR^{A24}, wherein R^{A24} is hydrogen or substituted or unsubstituted C₁₋₆alkyl.

In some embodiments, R^{24b} is hydrogen, substituted or unsubstituted C₁₋₆alkyl group, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, or substituted or unsubstituted 5- to 10-membered heteroaryl.

In some embodiments, R^{24b} is hydrogen or substituted or unsubstituted C₁₋₆alkyl. In some embodiments, R^{24b} is hydrogen or unsubstituted C₁₋₆alkyl. In some embodiments, R^{24b} is hydrogen. In some embodiments R^{24b} is unsubstituted C₁₋₆alkyl. In some embodiments, R^{24b} is —CH₃ or —CF₃. In some embodiments, R^{24b} is —CH₃.

In some embodiments, R^{24a} is —CF₃ and R^{24b} is hydrogen or C₁₋₄alkyl. In some embodiments, R^{24a} is a non-hydrogen group substituted with fluorine and R^{24b} is —CH₃.

In some embodiments, R^{24a} is a substituted or unsubstituted C₂₋₄alkyl, substituted or unsubstituted C₂₋₃alkenyl, substituted or unsubstituted C₂₋₃ alkynyl, or substituted or unsubstituted C₃carbocyclyl and R^{24b} is —CH₃. In some embodiments, R^{24a} is a substituted or unsubstituted C₁₋₄ alkyl, substituted or unsubstituted C₂₋₃alkenyl, substituted or unsubstituted C₂₋₃ alkynyl, or substituted or unsubstituted C₃carbocyclyl and R^{24b} is —H. In some embodiments, R^{24a} is a unsubstituted C₂₋₄alkyl, unsubstituted C₂₋₃alkenyl, or unsubstituted C₂₋₃alkynyl, or unsubstituted C₃carbocyclyl and R^{24b} is —H. In some embodiments, R^{24a} is unsubstituted C₂₋₄alkyl, unsubstituted C₂₋₃alkenyl, unsubstituted C₂₋₃ alkynyl, or unsubstituted C₃carbocyclyl; and R^{24b} is —CH₃. In some embodiments, when R^{24b} is H, R^{24a} is not C₁₋₆alkyl substituted with CN.

In some embodiments, R^{24a} is a non-hydrogen group substituted with fluorine, and R^{24b} is —CH₃.

In some embodiments, R^{24a} is —CH(CH₃)₂ or —CF₃, and R^{24b} is hydrogen.

In some embodiments, R^{24a} and R^{24b}, taken together with the carbon atom to which they are attached, form a substituted or unsubstituted C₃₋₆carbocyclyl, or a substituted or unsubstituted 3- to 10-membered heterocyclyl. In some embodiments, R^{24a} and R^{24b}, taken together with the carbon atom to which they are attached, form a substituted or unsubstituted C₃₋₆carbocyclyl. In some embodiments, R^{24a} and R^{24b}, taken together with the carbon atom to which they are attached, form an unsubstituted C₃₋₆carbocyclyl. In some embodiments, R^{24a} and R^{24b} taken together with the carbon atom to which they are attached, form a cyclopropyl. In some embodiments, R^{24a} and R^{24b} taken together with the carbon atoms to which they are attached, form an unsubstituted 3- to 10-membered heterocyclyl. In some embodiments, R^{24a} and R^{24b} taken together with the carbon atoms to which they are attached, form an unsubstituted 3- to 6-membered heterocyclyl.

In some embodiments, R³ is C₁₋₃ alkyl, R^{24a} is a non-hydrogen group substituted with fluorine, and R^{24b} is —CH₃.

In some embodiments, R³ is C₁₋₃ alkyl, R^{24a} is a non-hydrogen group substituted with fluorine, and R^{24b} is hydrogen.

In some embodiments, R³ is C₁₋₃ alkyl, R^{24a} is selected from the group consisting of substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, and R^{24b} is —CH₃. In some embodiments, R³ is C₁₋₃ alkyl; R^{24a} is selected from the group consisting of substituted or unsubstituted C₁₋₆alkyl, unsubstituted C₂₋₆alkenyl, unsubstituted C₂₋₆alkynyl, or unsubstituted C₃₋₆carbocyclyl; and R^{24b} is —CH₃. In some embodiments, R³ is —CH₃ or CH₂CH₃; R^{24a} is selected from the group consisting of substituted or unsubstituted C₁₋₆alkyl, unsubstituted C₂₋₆alkenyl, unsubstituted C₂₋₆alkynyl, or unsubstituted C₃₋₆carbocyclyl; and and R^{24b} is —CH₃. In some embodiments, R³ is C₁₋₃ alkyl; R^{24a} is selected from a substituted or unsubstituted C₁₋₆alkyl; and R^{24b} is —CH₃. In some embodiments, R³ is CH₃ or —CH₂CH₃; R^{24a} is selected from a substituted or unsubstituted C₁₋₆alkyl; and R^{24b} is —CH₃.

In some embodiments, R³ is —CH₃ or —CH₂CH₃ and at least one of R^{23a} and R^{23b} is fluorine or R^{23a} and R^{23b} are both hydrogen.

In some embodiments, R³ is —CH₃ or —CH₂CH₃ and R^{24a} is a non-hydrogen group substituted with fluorine or one or more —OR^{A24} groups, wherein R^{A24} is hydrogen or substituted or unsubstituted C₁₋₆alkyl.

In some embodiments, R³ is —CH₃ or —CH₂CH₃ and R^{24b} is —CH₃ or —CF₃. In some embodiments, R³ is —CH₃ or —CH₂CH₃; R^{24b} is —CH₃ or —CF₃; and R^{24a} is selected from the group consisting of substituted or unsubstituted C₁₋₆alkyl, unsubstituted C₂₋₆alkenyl, unsubstituted C₂₋₆alkynyl, or unsubstituted C₃₋₆carbocyclyl.

In some embodiments, R³ is —CH₃ or —CH₂CH₃, R^{24a} is —CH(CH₃)₂ or —CF₃; and R^{24b} is hydrogen.

q, r and s—as they Relate to Formulae (D-I)-(D-IVb-2ii)

In some embodiments, q is selected from 0, 1 or 2, and r is selected from 1 or 2, provided that a) q and r are not both 1, or b) when q is 0, r is not 2. In some embodiments, q and r are not both 1. In some embodiments, when q is 0, r is not 2. In some embodiments q is 0. In some embodiments q is 1. In some embodiments, q is 2. In some embodiments r is 1. In some embodiments, r is 2. In some embodiments, q is 1 and r is 2. In some embodiments, q is 2 and r is 1. In some embodiments, q is 0 and r is 1. In some embodiments, q is 0 and r is 2. In some embodiments, q is 2 and r is 2.

In some embodiments, s is 1 or 2. In some embodiments, s is 1. In some embodiments, s is 2. In some embodiments, s is 3. In some embodiments, s is 4.

In some embodiments, q is 1, r is 2 and s is 1. In some embodiments, q is 1, r is 2 and s is 2. In some embodiments, q is 1, r is 2 and s is 3. In some embodiments, q is 1, r is 2 and s is 4.

In some embodiments, q is 2, r is 1 and s is 1. In some embodiments, q is 2, r is 1 and s is 2. In some embodiments, q is 2, r is 1 and s is 3. In some embodiments, q is 2, r is 1 and s is 4.

In some embodiments, q is 0, r is 1 and s is 1. In some embodiments, q is 0, r is 1 and s is 2. In some embodiments, q is 0, r is 1 and s is 3. In some embodiments, q is 0, r is 1 and s is 4.

In some embodiments, q is 2, r is 2 and s is 1. In some embodiments, q is 2, r is 2 and s is 2. In some embodiments, q is 2, r is 2 and s is 3. In some embodiments, q is 2, r is 2, and s is 4.

In some embodiments, the compound of Formula (D-I) is any one of compounds D-1, D-2, D-3, D-4, D-5, D-6, D-7, D-8, D-9, D-9a, D-10, D-11, D-12, D-13, D-14, D-15, D-16, or D-17 in Table D-1 or pharmaceutically acceptable salts thereof. In some embodiments, the compound of Formula (D-I) is any one of compounds D-1, D-2, D-3, D-4, D-5, D-6, D-7, D-8, D-9, D-9a, D-10, D-11, D-12, D-13, D-14, D-15, D-16, or D-17 in Table D-1. In some embodiments, the compound of Formula (D-I) is a pharmaceutically acceptable salt of any one of Compounds D-1, D-2, D-3, D-4, D-5, D-6, D-7, D-8, D-9, D-9a, D-10, D-11, D-12, D-13, D-14, D-15, D-16, or D-17. In some embodiments, the compound of Formula (D-I) is selected from any one of compounds D-1, D-2, D-3, D-4, D-5, D-6, D-7, D-8, D-9, D-10, D-11, D-12, D-13, D-14, D-15, D-16, or D-17 in Table D-1 or pharmaceutically acceptable salts thereof. In some embodiments, the compound of Formula (D-I) is selected from any one of compounds D-1, D-2, D-3, D-4, D-5, D-6, D-7, D-8, D-9, D-10, D-11, D-12, D-13, D-14, D-15, D-16, or D-17 in Table D-1. In some embodiments, the compound of Formula (D-I) is a pharmaceutically acceptable salt of any one of Compounds D-1, D-2, D-3, D-4, D-5, D-6, D-7, D-8, D-9, D-10, D-11, D-12, D-13, D-14, D-15, D-16, or D-17 in Table D-1.

TABLE D-1

| Cmpd | Structure |
|---|---|
| D-1 | 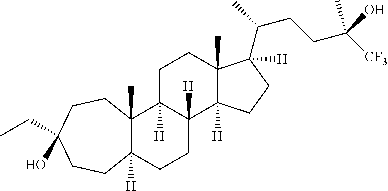 |
| D-2 | 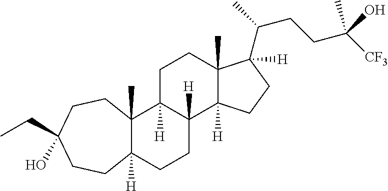 |
| D-3 | 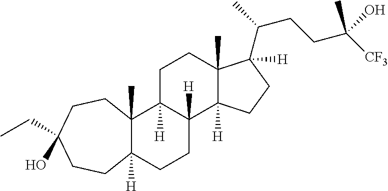 |
| D-4 | 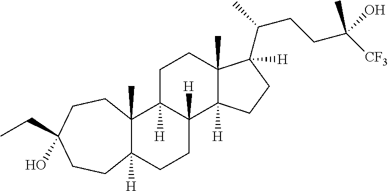 |
| D-5 | 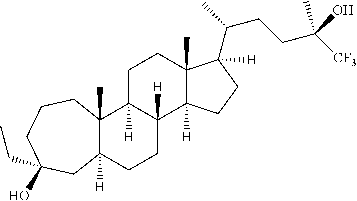 |
| D-6 | 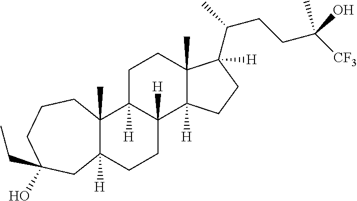 |
| D-7 | 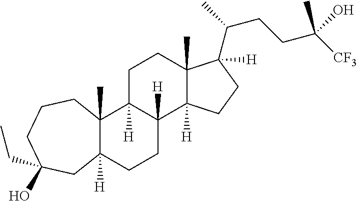 |
| D-8 | 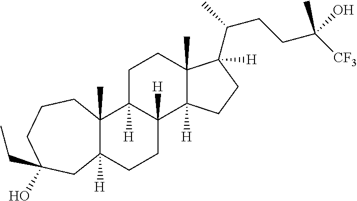 |
| D-9 | 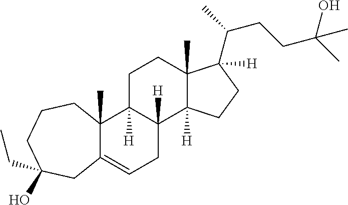 |
| D-9a | 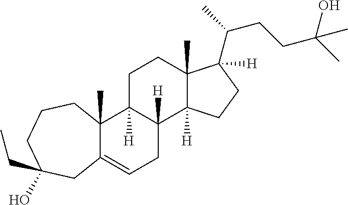 |
| D-10 | 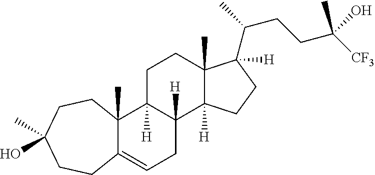 |
| D-11 | 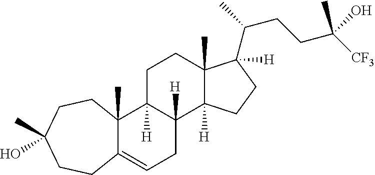 |
| D-12 | 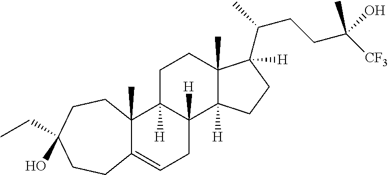 |
| D-13 | 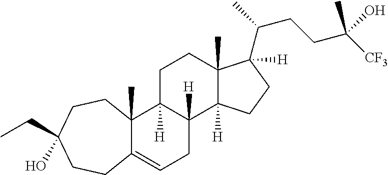 |
| D-14 | 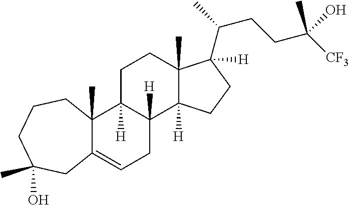 |
| D-15 | 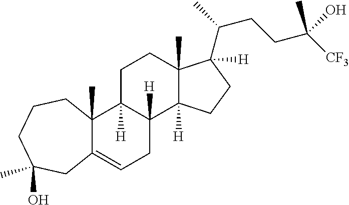 |
| D-16 | 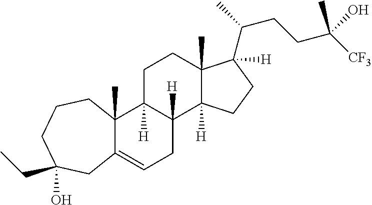 |
| D-17 | 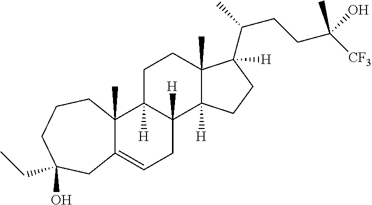 |

In one aspect, the present disclosure provides a compound of Formula (E-I):

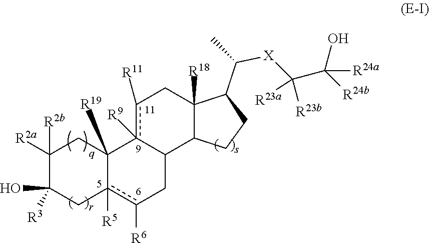

or a pharmaceutically acceptable salt thereof;
wherein:
   each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein R^{A2} is hydrogen or substituted or unsubstituted C₁₋₆alkyl;
   R³ is substituted or unsubstituted C₁₋₆alkyl, substituted or unsubstituted C₂₋₆alkenyl, or substituted or unsubstituted C₂₋₆alkynyl, or substituted or unsubstituted C₃₋₁₀carbocyclyl;
   [symbol] represents a single or double bond;
   when the bond between C9 and C11 is a double bond, R⁹ is absent and R¹¹ is selected from the group consisting of hydrogen, halogen, substituted or unsubstituted C₁₋₆alkyl, substituted or unsubstituted C₂₋₆alkenyl, substituted or unsubstituted C₂₋₆alkynyl, substituted or unsubstituted C₃₋₁₀carbocyclyl, substituted or unsubstituted C₁₋₆alkoxy, substituted or unsubstituted 3- to 10-membered heterocyclyl, —OH, and -amino;
   when the bond between C9 and C11 is a single bond, then R⁹ is hydrogen and R¹¹ is halogen or substituted or unsubstituted C₁₋₆alkyl; or R⁹ and R¹¹, taken together with the carbon atoms to which they are attached, form a substituted or unsubstituted C₃₋₆carbocyclyl ring;
   when the bond between C5 and C6 is a double bond, then R⁵ is absent and R⁶ is hydrogen, halogen, or substituted or unsubstituted C₁₋₆alkyl;
   when the bond between C5 and C6 is a single bond, then each of R⁵ and R⁶ is independently hydrogen, halogen, or substituted or unsubstituted C₁₋₆alkyl;
   R¹⁸ is hydrogen, substituted or unsubstituted C₁₋₆alkyl, or OR^{A18}, wherein R^{A18} is substituted or unsubstituted C₁₋₆alkyl;
   R¹⁹ is hydrogen, substituted or unsubstituted C₁₋₆alkyl, or OR^{A19}, wherein R^{A19} is substituted or unsubstituted C₁₋₆alkyl;
   X is —(C(R^{X})₂)ₙ— or —O—, wherein R^{X} is hydrogen or fluorine, or one R^{X} group and R^{23b} are joined to form a double bond;
   n is 1 or 2;
   each of R^{23a} and R^{23b} is independently hydrogen or fluorine;
   R^{24a} is selected from the group consisting of substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl;
   R^{24b} is hydrogen or a substituted or unsubstituted C₁₋₆alkyl; or
   R^{24a} and R^{24b}, taken together with the carbon atoms to which they are attached, form a substituted or unsubstituted C₃₋₆carbocyclyl, or substituted or unsubstituted 3 to 6-membered heterocyclyl;
   q is 0, 1 or 2;
   r is 1 or 2; and
   s is 1 or 2.

In one aspect, the present disclosure provides a compound of Formula (E-Ia):

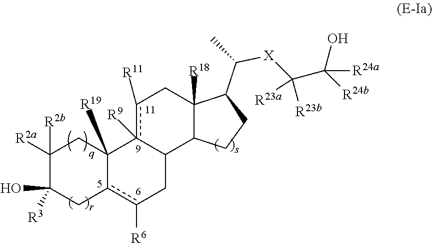

or a pharmaceutically acceptable salt thereof;
wherein:
   each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein R^{A2} is hydrogen or substituted or unsubstituted C₁₋₆alkyl;
   R³ is substituted or unsubstituted C₁₋₆alkyl, substituted or unsubstituted C₂₋₆ alkenyl, or substituted or unsubstituted C₂₋₆alkynyl, or substituted or unsubstituted C₃₋₁₀ carbocyclyl;
   [symbol] represents a single or double bond;
   when the bond between C9 and C11 is a double bond, R⁹ is absent and R¹¹ is selected from the group consisting of hydrogen, halogen, substituted or unsubstituted C₁₋₆alkyl, substituted or unsubstituted C₂₋₆alkenyl, substituted or unsubstituted C₂₋₆ alkynyl, substituted or unsubstituted C₃₋₁₀carbocyclyl, substituted or unsubstituted C₁₋₆alkoxy, substituted or unsubstituted 3- to 10-membered heterocyclyl, —OH, and -amino;
   when the bond between C9 and C11 is a single bond, then R⁹ is hydrogen and R¹¹ is halogen or substituted or unsubstituted C₁₋₆alkyl; or R⁹ and R¹¹, taken together with the carbon atoms to which they are attached, form a substituted or unsubstituted C₃₋₆carbocyclyl ring;
   R⁶ is hydrogen, substituted or unsubstituted C₁₋₆alkyl, or halogen; provided if a single bond is present between C5 and C6, then the hydrogen at C5 is in the alpha configuration;
   R¹⁸ is hydrogen, substituted or unsubstituted C₁₋₆alkyl, or OR^{A18}, wherein R^{A18} is substituted or unsubstituted C₁₋₆alkyl;
   R¹⁹ is hydrogen, substituted or unsubstituted C₁₋₆alkyl, or OR^{A19}, wherein R^{A19} is substituted or unsubstituted C₁₋₆alkyl;
   X is —(C(R^{X})₂)ₙ— or —O—, wherein R^{X} is hydrogen or fluorine, or one R^{X} group and R^{23b} are joined to form a double bond;
   n is 1 or 2;
   each of R^{23a} and R^{23b} is independently hydrogen or fluorine;
   R^{24a} is selected from the group consisting of substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl;
   R^{24b} is hydrogen or a substituted or unsubstituted C₁₋₆alkyl; or
   R^{24a} and R^{24b}, taken together with the carbon atoms to which they are attached, form a substituted or unsubstituted C₃₋₆carbocyclyl, or substituted or unsubstituted 3- to 6-membered heterocyclyl;
   q is 0, 1 or 2;
   r is 1 or 2; and
   s is 1 or 2.

In some embodiments, the compound of formula (E-I) is a compound of formula (E-I-A):

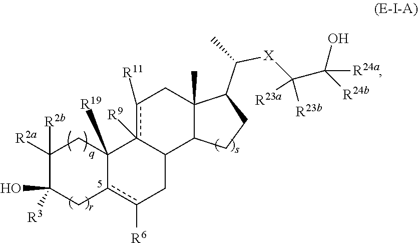

or a pharmaceutically acceptable salt thereof.

In some embodiments, the compound of formula (E-I) is a compound of formula (E-I-B):

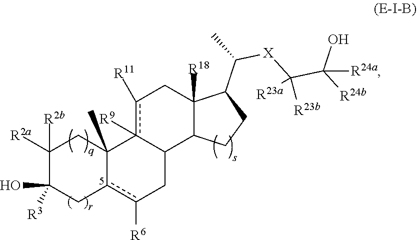

or a pharmaceutically acceptable salt thereof.

In some embodiments, the compound of formula (E-I) is a compound of formula (E-I-C):

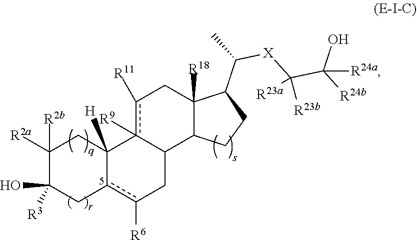

or a pharmaceutically acceptable salt thereof.

In some embodiments, the compound of formula (E-I) is a compound of formula (E-I-D):

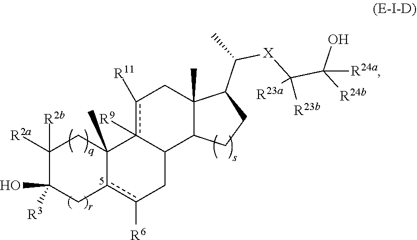

or a pharmaceutically acceptable salt thereof.

In some embodiments, the compound of formula (E-I-D) is a compound of formula (E-I-D-i):

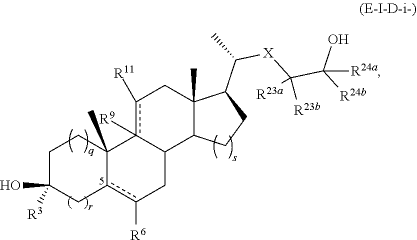

or a pharmaceutically acceptable salt thereof.

In some embodiments, the compound of formula (E-I-D-i) is a compound of formula (E-I-D-i-1):

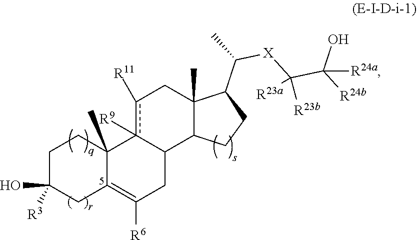

or a pharmaceutically acceptable salt thereof.

In some embodiments, the compound of formula (E-I-D-i-1) is a compound of formula (E-I-D-i-1a):

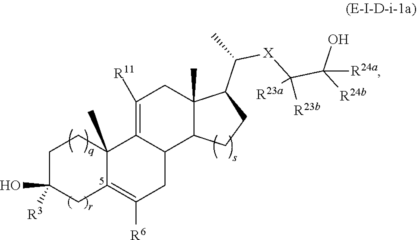

or a pharmaceutically acceptable salt thereof.

In some embodiments, the compound of formula (E-I-D-i-1) is a compound of formula (E-I-D-i-1b):

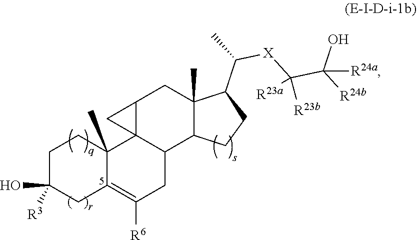

or a pharmaceutically acceptable salt thereof.

In some embodiments, the compound of formula (E-I-D-i) is a compound of formula (E-I-D-i-2):

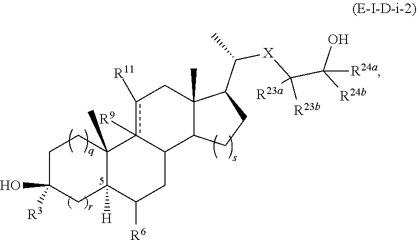

or a pharmaceutically acceptable salt thereof.

In some embodiments, the compound of formula (E-I-D-i-2) is a compound of formula (E-I-D-i-2a):

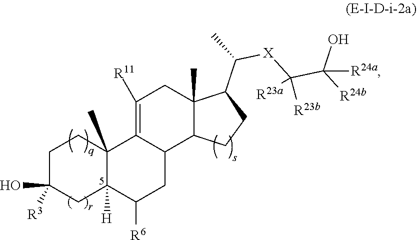

or a pharmaceutically acceptable salt thereof.

In some embodiments, the compound of formula (E-I-D-i-2) is a compound of formula (E-I-D-i-2b):

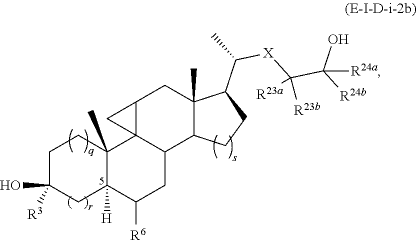

or a pharmaceutically acceptable salt thereof.

In some embodiments, the compound of formula (E-I) is a compound of formula (E-I-E):

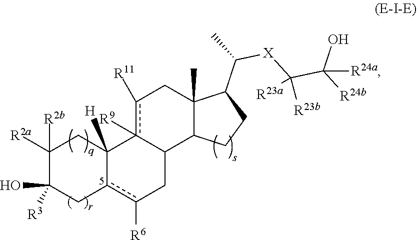

or a pharmaceutically acceptable salt thereof.

In some embodiments, the compound of formula (E-I-E) is a compound of formula (E-I-E-i):

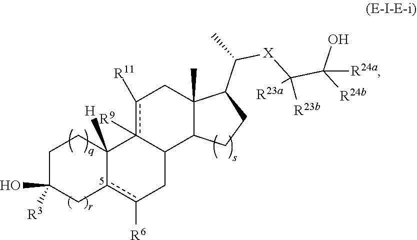

or a pharmaceutically acceptable salt thereof.

In some embodiments, the compound of formula (E-I-E-i) is a compound of formula (E-I-E-i-1):

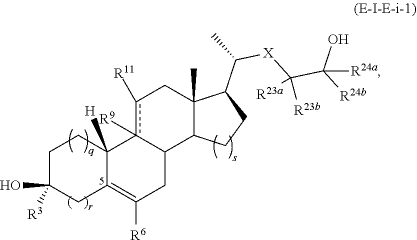

or a pharmaceutically acceptable salt thereof.

In some embodiments, the compound of formula (E-I-E-i-1) is a compound of formula (E-I-E-i-1a):

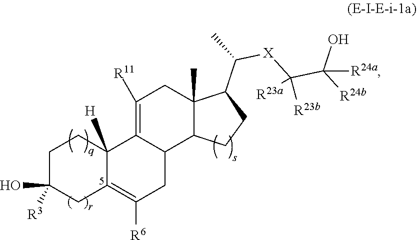

or a pharmaceutically acceptable salt thereof.

In some embodiments, the compound of formula (E-I-E-i-1) is a compound of formula (E-I-E-i-1b):

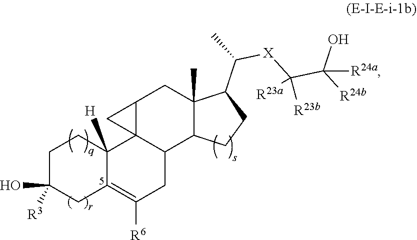

or a pharmaceutically acceptable salt thereof.

In some embodiments, the compound of formula (E-I-E-i) is a compound of formula (E-I-E-i-2):

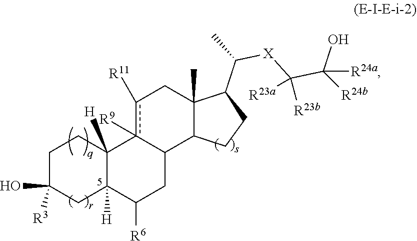

or a pharmaceutically acceptable salt thereof.

In some embodiments, the compound of formula (E-I-E-i-2) is a compound of formula (E-I-E-i-2a):

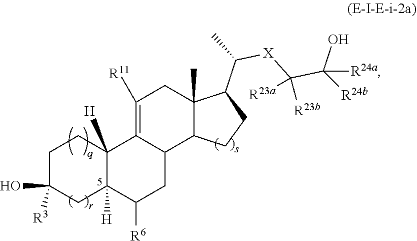

or a pharmaceutically acceptable salt thereof.

In some embodiments, the compound of formula (E-I-E-i-2) is a compound of formula (E-I-E-i-2b):

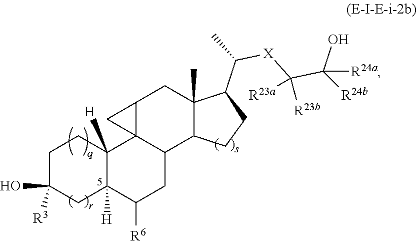

or a pharmaceutically acceptable salt thereof.

In some embodiments, the compound of formula (E-I) is a compound of formula (E-II):

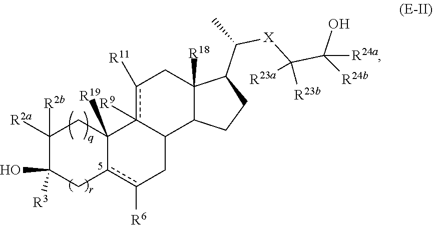

or a pharmaceutically acceptable salt thereof.

In some embodiments, the compound of formula (E-II) is a compound of formula (E-II-A):

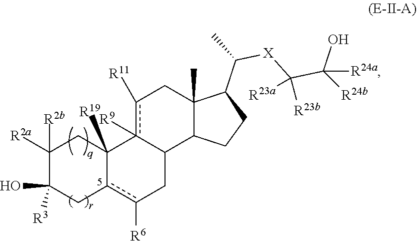

or a pharmaceutically acceptable salt thereof.

In some embodiments, the compound of formula (E-II) is a compound of formula (E-II-B):

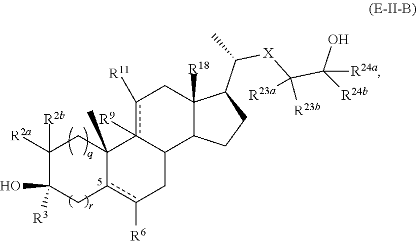

or a pharmaceutically acceptable salt thereof.

In some embodiments, the compound of formula (E-II) is a compound of formula (E-II-C):

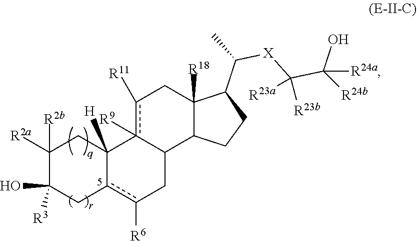

or a pharmaceutically acceptable salt thereof.

In some embodiments, the compound of formula (E-II) is a compound of formula (E-II-D):

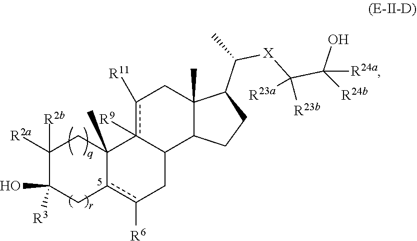

or a pharmaceutically acceptable salt thereof.

In some embodiments, the compound of formula (E-II-D) is a compound of formula (E-II-D-i):

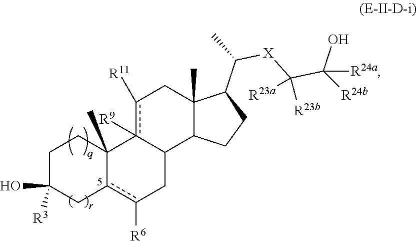

or a pharmaceutically acceptable salt thereof.

In some embodiments, the compound of formula (E-II-D-i) is a compound of formula (E-II-D-i-1):

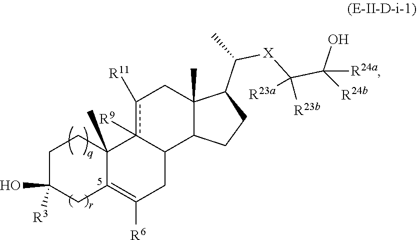

or a pharmaceutically acceptable salt thereof.

In some embodiments, the compound of formula (E-II-D-i-1) is a compound of formula (E-II-D-i-1a):

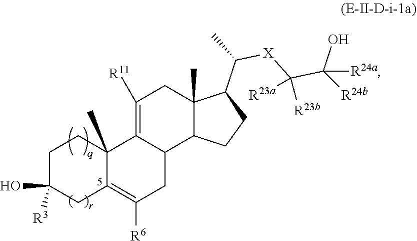

or a pharmaceutically acceptable salt thereof.

In some embodiments, the compound of formula (E-II-D-i-1) is a compound of formula (E-II-D-i-1b):

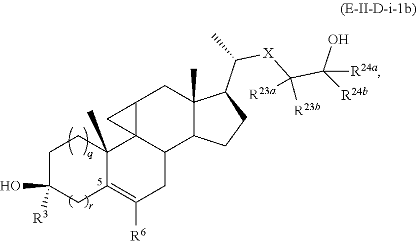

or a pharmaceutically acceptable salt thereof.

In some embodiments, the compound of formula (E-II-D-i) is a compound of formula (E-II-D-i-2):

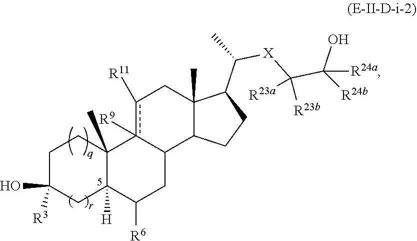

or a pharmaceutically acceptable salt thereof.

In some embodiments, the compound of formula (E-II-D-i-2) is a compound of formula (E-II-D-i-2a):

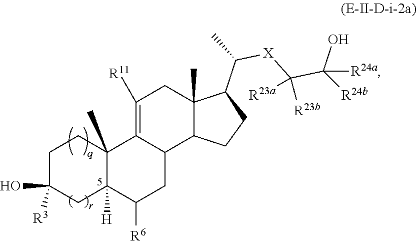

or a pharmaceutically acceptable salt thereof.

In some embodiments, the compound of formula (E-II-D-i-2) is a compound of formula (E-II-D-i-2b):

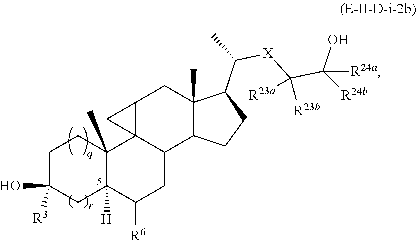

or a pharmaceutically acceptable salt thereof.

In some embodiments, the compound of formula (E-II) is a compound of formula (E-II-E):

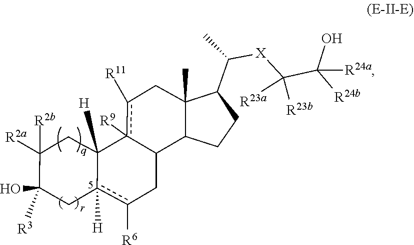

or a pharmaceutically acceptable salt thereof.

In some embodiments, the compound of formula (E-II-E) is a compound of formula (E-II-E-i):

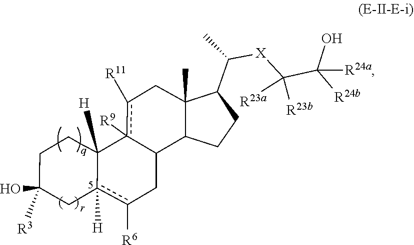

or a pharmaceutically acceptable salt thereof.

In some embodiments, the compound of formula (E-II-E-i) is a compound of formula (E-II-E-i-1):

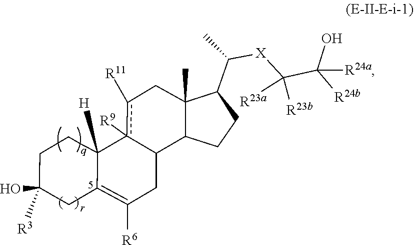

or a pharmaceutically acceptable salt thereof.

In some embodiments, the compound of formula (E-II-E-i-1) is a compound of formula (E-II-E-i-1a):

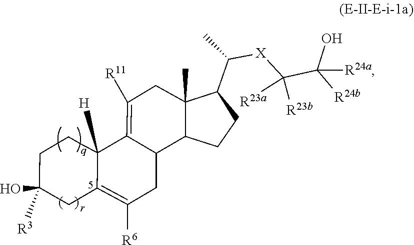

or a pharmaceutically acceptable salt thereof.

In some embodiments, the compound of formula (E-II-E-i-1) is a compound of formula (E-II-E-i-1b):

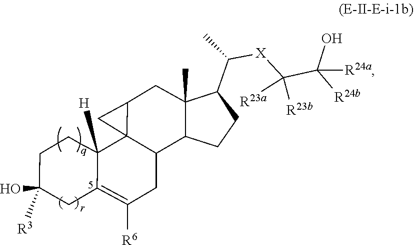

or a pharmaceutically acceptable salt thereof.

In some embodiments, the compound of formula (E-II-E-i) is a compound of formula (E-II-E-i-2):

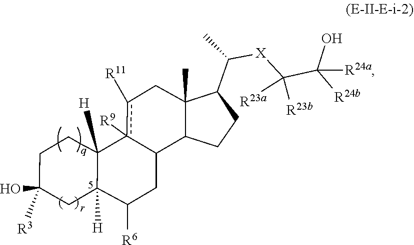

or a pharmaceutically acceptable salt thereof.

In some embodiments, the compound of formula (E-II-E-i-2) is a compound of formula (E-II-E-i-2a):

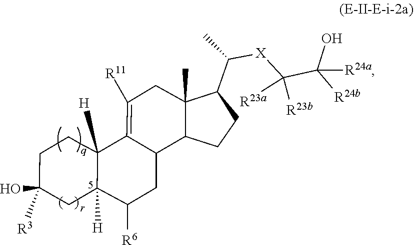

or a pharmaceutically acceptable salt thereof.

In some embodiments, the compound of formula (E-II-E-i-2) is a compound of formula (E-II-E-i-2b):

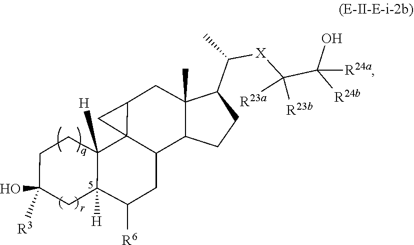

or a pharmaceutically acceptable salt thereof.

In some embodiments, the compound of formula (E-I) is a compound of formula (E-III):

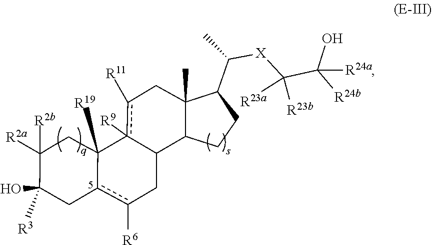

or a pharmaceutically acceptable salt thereof.

In some embodiments, the compound of formula (E-III) is a compound of formula (E-III-A):

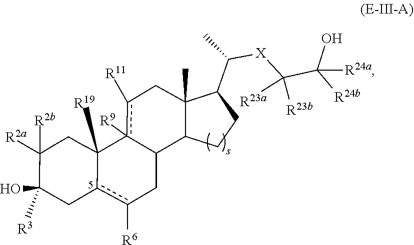

or a pharmaceutically acceptable salt thereof.

In some embodiments, the compound of formula (E-III) is a compound of formula (E-III-B):

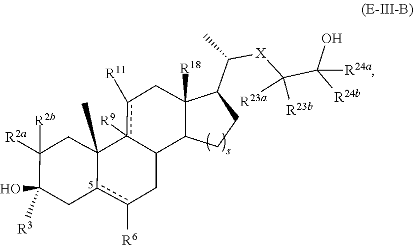

or a pharmaceutically acceptable salt thereof.

In some embodiments, the compound of formula (E-III) is a compound of formula (E-III-C):

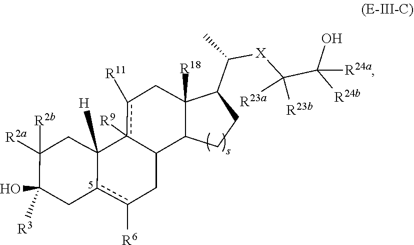

or a pharmaceutically acceptable salt thereof.

In some embodiments, the compound of formula (E-III) is a compound of formula (E-III-D):

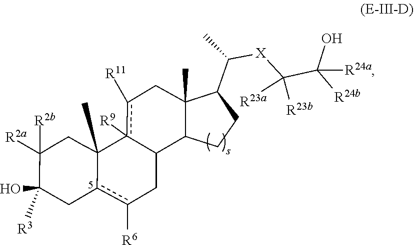

or a pharmaceutically acceptable salt thereof.

In some embodiments, the compound of formula (E-III-D) is a compound of formula (E-III-D-i):

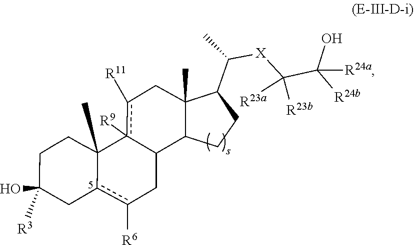

or a pharmaceutically acceptable salt thereof.

In some embodiments, the compound of formula (E-III-D-i) is a compound of formula (E-III-D-i-1):

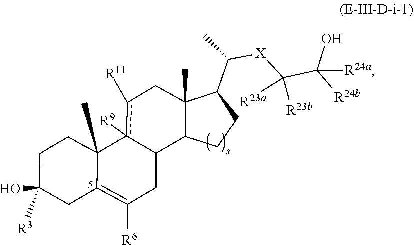

or a pharmaceutically acceptable salt thereof.

In some embodiments, the compound of formula (E-III-D-i-1) is a compound of formula (E-III-D-i-1a):

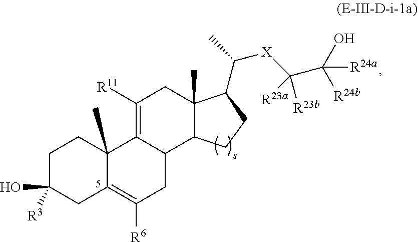

or a pharmaceutically acceptable salt thereof.

In some embodiments, the compound of formula (E-III-D-i-1) is a compound of formula (E-III-D-i-1b):

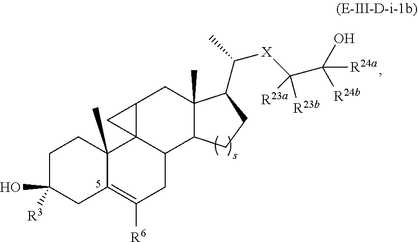

or a pharmaceutically acceptable salt thereof.

In some embodiments, the compound of formula (E-III-D-i) is a compound of formula (E-III-D-i-2):

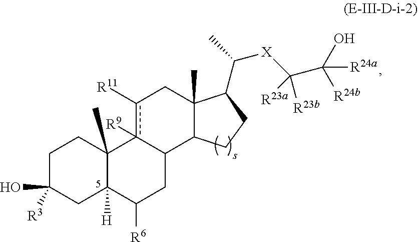

or a pharmaceutically acceptable salt thereof.

In some embodiments, the compound of formula (E-III-D-i-2) is a compound of formula (E-III-D-i-2a):

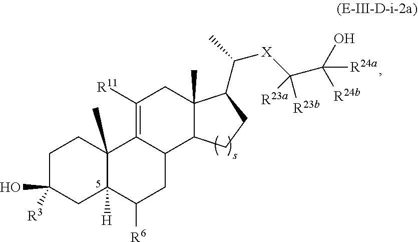

or a pharmaceutically acceptable salt thereof.

In some embodiments, the compound of formula (E-III-D-i-2) is a compound of formula (E-III-D-i-2b):

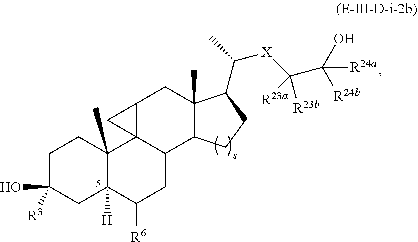

or a pharmaceutically acceptable salt thereof.

In some embodiments, the compound of formula (E-III) is a compound of formula (E-III-E):

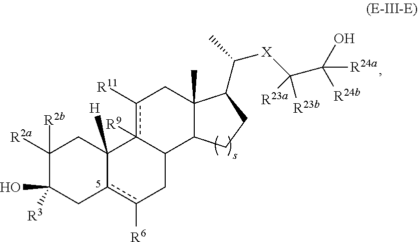

or a pharmaceutically acceptable salt thereof.

In some embodiments, the compound of formula (E-III-E) is a compound of formula (E-III-E-i):

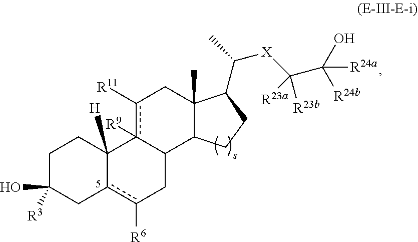

or a pharmaceutically acceptable salt thereof.

In some embodiments, the compound of formula (E-III-E-i) is a compound of formula (E-III-E-i-1):

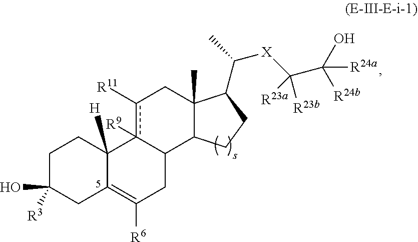

or a pharmaceutically acceptable salt thereof.

In some embodiments, the compound of formula (E-III-E-i-1) is a compound of formula (E-III-E-i-1a):

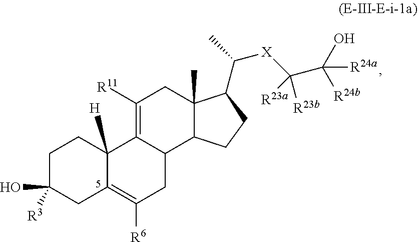

or a pharmaceutically acceptable salt thereof.

In some embodiments, the compound of formula (E-III-E-i-1) is a compound of formula (E-III-E-i-1b):

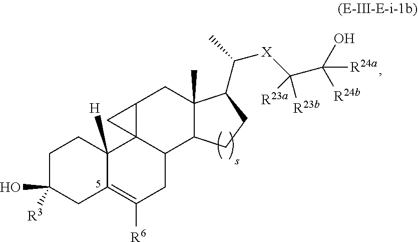

or a pharmaceutically acceptable salt thereof.

In some embodiments, the compound of formula (E-III-E-i) is a compound of formula (E-III-E-i-2):

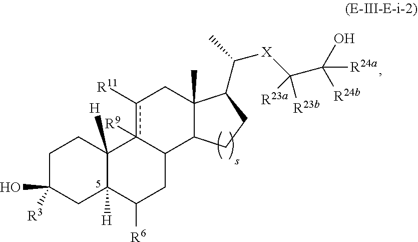

or a pharmaceutically acceptable salt thereof.

In some embodiments, the compound of formula (E-III-E-i-2) is a compound of formula (E-III-E-i-2a):

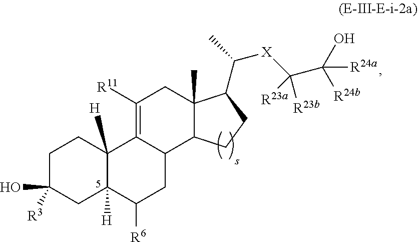

or a pharmaceutically acceptable salt thereof.

In some embodiments, the compound of formula (E-III-E-i-2) is a compound of formula (E-III-E-i-2b):

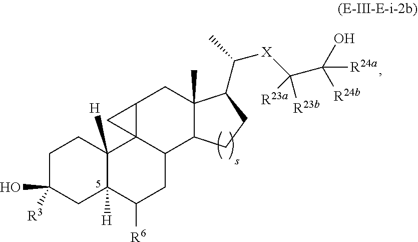

or a pharmaceutically acceptable salt thereof.

In some embodiments, the compound of formula (E-I) is a compound of formula (E-IV):

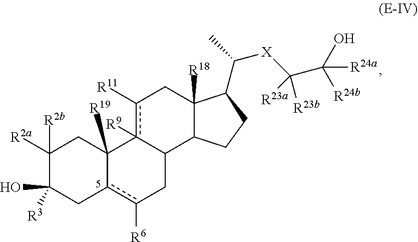

or a pharmaceutically acceptable salt thereof.

In some embodiments, the compound of formula (E-IV) is a compound of formula (E-IV-A):

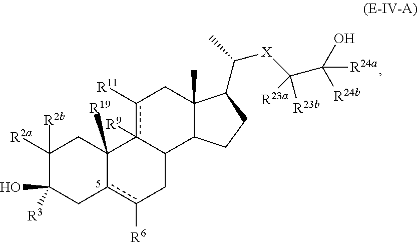

or a pharmaceutically acceptable salt thereof.

In some embodiments, the compound of formula (E-IV) is a compound of formula (E-IV-B):

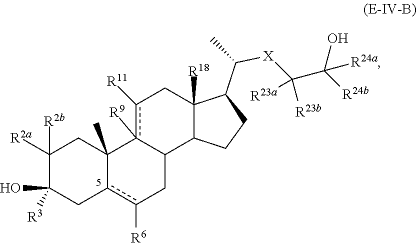

or a pharmaceutically acceptable salt thereof.

In some embodiments, the compound of formula (E-IV) is a compound of formula (E-IV-C):

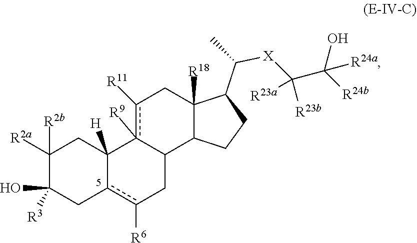

or a pharmaceutically acceptable salt thereof.

In some embodiments, the compound of formula (E-IV) is a compound of formula (E-IV-D):

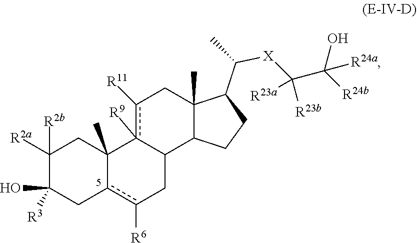

or a pharmaceutically acceptable salt thereof.

In some embodiments, the compound of formula (E-IV-D) is a compound of formula (E-IV-D-i):

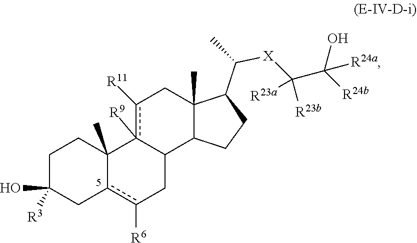

or a pharmaceutically acceptable salt thereof.

In some embodiments, the compound of formula (E-IV-D-i) is a compound of formula (E-IV-D-i-1):

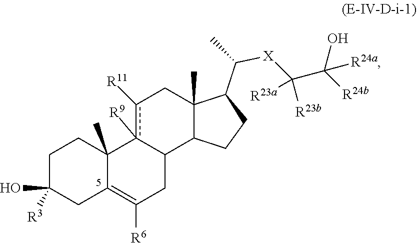

or a pharmaceutically acceptable salt thereof.

In some embodiments, the compound of formula (E-IV-D-i-1) is a compound of formula (E-IV-D-i-1a):

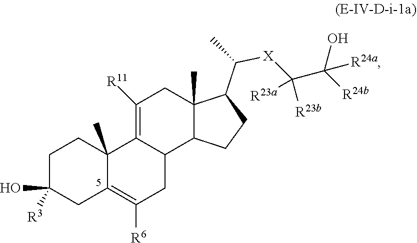

or a pharmaceutically acceptable salt thereof.

In some embodiments, the compound of formula (E-IV-D-i-1) is a compound of formula (E-IV-D-i-1b):

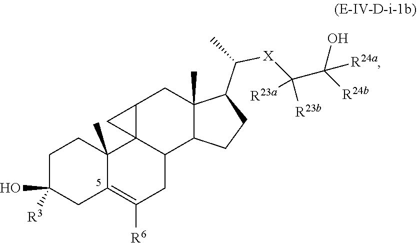

or a pharmaceutically acceptable salt thereof.

In some embodiments, the compound of formula (E-IV-D-i) is a compound of formula (E-IV-D-i-2):

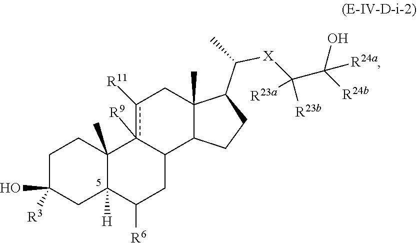

or a pharmaceutically acceptable salt thereof.

In some embodiments, the compound of formula (E-IV-D-i-2) is a compound of formula (E-IV-D-i-2a):

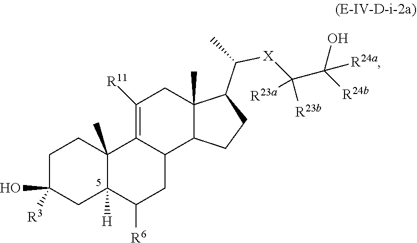

or a pharmaceutically acceptable salt thereof.

In some embodiments, the compound of formula (E-IV-D-i-2) is a compound of formula (E-IV-D-i-2b):

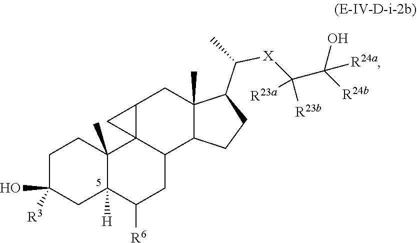

or a pharmaceutically acceptable salt thereof.

In some embodiments, the compound of formula (E-IV) is a compound of formula (E-IV-E):

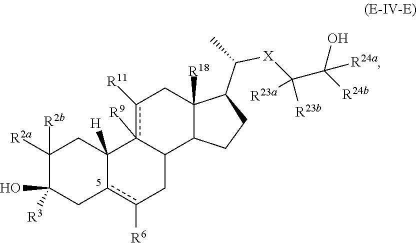

or a pharmaceutically acceptable salt thereof.

In some embodiments, the compound of formula (E-IV-E) is a compound of formula (E-IV-E-i):

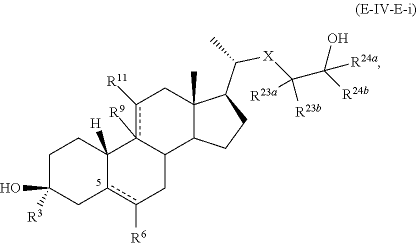

or a pharmaceutically acceptable salt thereof.

In some embodiments, the compound of formula (E-IV-E-i) is a compound of formula (E-IV-E-i-1):

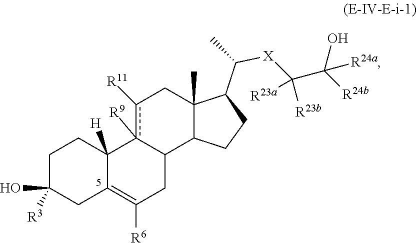

or a pharmaceutically acceptable salt thereof.

In some embodiments, the compound of formula (E-IV-E-i-1) is a compound of formula (E-IV-E-i-1a):

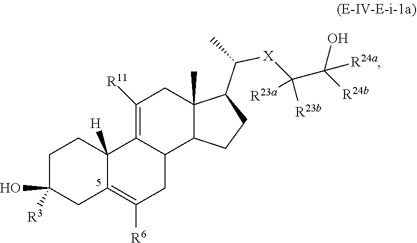

or a pharmaceutically acceptable salt thereof.

In some embodiments, the compound of formula (E-IV-E-i-1) is a compound of formula (E-IV-E-i-1b):

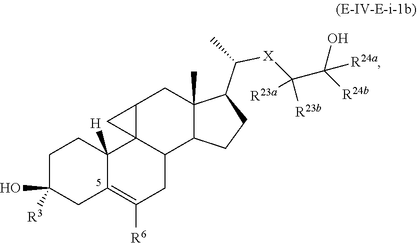

or a pharmaceutically acceptable salt thereof.

In some embodiments, the compound of formula (E-IV-E-i) is a compound of formula (E-IV-E-i-2):

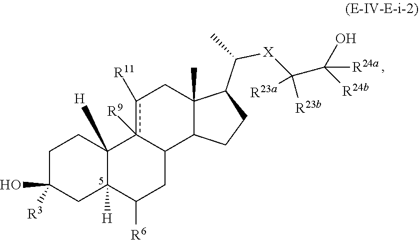

or a pharmaceutically acceptable salt thereof.

In some embodiments, the compound of formula (E-IV-E-i-2) is a compound of formula (E-IV-E-i-2a):

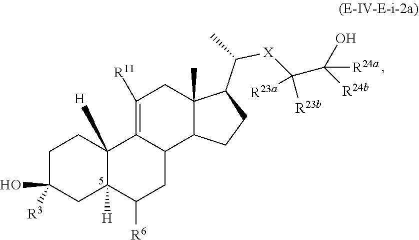

or a pharmaceutically acceptable salt thereof.

In some embodiments, the compound of formula (E-IV-E-i-2) is a compound of formula (E-IV-E-i-2b):

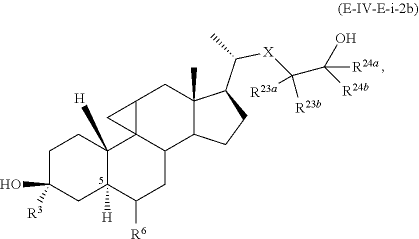

or a pharmaceutically acceptable salt thereof.
   Groups R^{2a} and R^{2b}—as they Relate to Formulae (E-I)-(E-IV-E-i-2b)

In certain embodiments, each of R^{2a} and R^{2b} is independently hydrogen, halogen, substituted or unsubstituted C₁₋₆alkyl, substituted or unsubstituted C₃₋₁₀carbocyclyl, or OR^{A2}, wherein R^{A2} is hydrogen or substituted or unsubstituted C₁₋₆alkyl. In certain embodiments, each of R^{2a} and R^{2b} is hydrogen. In certain embodiments, each of R^{2a} and R^{2b} is independently halogen, e.g., fluoro, chloro, bromo, or iodo. In certain embodiments, each of R^{2a} and R^{2b} is independently fluoro or chloro. In certain embodiments, each of R^{2a} and R^{2b} is independently is substituted or unsubstituted C₁₋₆alkyl, e.g., substituted or unsubstituted C₁₋₂alkyl, substituted or unsubstituted C₂₋₃ alkyl, substituted or unsubstituted C₃₋₄ alkyl, substituted or unsubstituted C₄₋₅ alkyl, or substituted or unsubstituted C₅₋₆ alkyl. In certain embodiments, each of R^{2a} and R^{2b} is independently —CH₃, —CH₂CH₃, —CH₂CH₂CH₃, or cyclopropyl. In certain embodiments, each of R^{2a} and R^{2b} is independently —OR^{A2}. In certain embodiments, R^{A2} is hydrogen. In certain embodiments, R^{A2} is substituted or unsubstituted alkyl, e.g., substituted or unsubstituted C₁₋₆alkyl, substituted or unsubstituted C₁₋₂ alkyl, substituted or unsubstituted C₂₋₃ alkyl, substituted or unsubstituted C₃₋₄ alkyl, substituted or unsubstituted C₄₋₅alkyl, or substituted or unsubstituted C₅₋₆ alkyl. In certain embodiments, R^{A2} is hydrogen, —CH₃, —CH₂CH₃, or —CH₂CH₂CH₃, i.e., to provide a group of R^{2a} or R^{2b} each independently of formula-OH, —OCH₃, —OCH₂CH₃, or —OCH₂CH₂CH₃. In certain embodiments, each of R^{2a} and R^{2b} is a non-hydrogen substituent. In certain embodiments, one of R^{2a} and R^{2b} is a non-hydrogen substituent in the alpha configuration. In certain embodiments, one of R^{2a} and R^{2b} is a non-hydrogen substituent in the beta configuration.

### Group R³—as it Relates to Formulae (E-I)-(E-IV-E-i-2b)

In certain embodiments, R³ is substituted or unsubstituted aliphatic, i.e., substituted or unsubstituted alkyl, substituted or unsubstituted alkenyl, substituted or unsubstituted alkynyl, or substituted or unsubstituted carbocyclyl.

In certain embodiments, R³ is substituted or unsubstituted C₁₋₆alkyl, substituted or unsubstituted C₂₋₆alkenyl, substituted or unsubstituted C₂₋₆alkynyl, or substituted or unsubstituted C₃₋₁₀carbocyclyl.

In certain embodiments, R³ is substituted or unsubstituted alkyl, e.g., substituted or unsubstituted C₁₋₆alkyl, substituted or unsubstituted C₁₋₂ alkyl, substituted or unsubstituted C₂₋₃ alkyl, substituted or unsubstituted C₃₋₄ alkyl, substituted or unsubstituted C₄₋₅alkyl, or substituted or unsubstituted C₅₋₆ alkyl. Exemplary R³ C₁₋₆alkyl groups include, but are not limited to, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), n-hexyl (C₆), C₁₋₆ alkyl substituted with 1, 2, 3, 4, 5, 6, 7, 8, 9, 10, or more fluoro groups (e.g., —CF₃, —CH₂F, —CHF₂, difluoroethyl, and 2,2,2-trifluoro-1,1-dimethyl-ethyl), C₁₋₆alkyl substituted with 1, 2, 3, 4, 5, 6, 7, 8, 9, 10, or more chloro groups (e.g., —CH₂Cl, —CHCl₂), and C₁₋₆alkyl substituted with 1, 2, 3, 4, 5, 6, 7, 8, 9, 10, or more alkoxy groups (e.g., —CH₂OCH₃, —CH₂OCH₂CH₃, —CH₂O-cyclopropyl). In certain embodiments, R³ is substituted alkyl, e.g., R³ is haloalkyl, alkoxyalkyl, or aminoalkyl. In certain embodiments, R³ is Me, Et, n-Pr, n-Bu, i-Bu, fluoromethyl, chloromethyl, difluoromethyl, trifluoromethyl, trifluoroethyl, difluoroethyl, 2,2,2-trifluoro-1,1-dimethyl-ethyl, methoxymethyl, methoxyethyl, or ethoxymethyl.

In certain embodiments, R³ is unsubstituted C₁₋₆alkyl. In some embodiments, R³ is selected from the group consisting of CH₃, —CH₂CH₃, or —CH₂CH₂CH₃. In some embodiments, R³ is ethyl.

In certain embodiments, R³ is alkyl substituted with one or more fluorine atoms; e.g., R³ is —CH₂F, —CHF₂, or —CF₃.

In certain embodiments, R³ is alkyl substituted with one or more —OR^{A3} groups, wherein R^{A3} is hydrogen or substituted or unsubstituted alkyl. In certain embodiments, R³ is —CH₂OR^{A3}, e.g., wherein R^{A3} is hydrogen, —CH₃, —CH₂CH₃, or —CH₂CH₂CH₃.

In certain embodiments, R³ is substituted or unsubstituted alkenyl, e.g., substituted or unsubstituted C₂₋₆alkenyl, substituted or unsubstituted C₂₋₃alkenyl, substituted or unsubstituted C₃₋₄ alkenyl, substituted or unsubstituted C₄₋₅alkenyl, or substituted or unsubstituted C₅₋₆ alkenyl. In certain embodiments, R³ is ethenyl (C₂), propenyl (C₃), or butenyl (C₄), unsubstituted or substituted with one or more substituents selected from the group consisting of alkyl, halo, haloalkyl, alkoxyalkyl, or hydroxyl. In certain embodiments, R³ is ethenyl, propenyl, or butenyl, unsubstituted or substituted with alkyl, halo, haloalkyl, alkoxyalkyl, or hydroxy. In certain embodiments, R³ is ethenyl.

In certain embodiments, R³ is substituted or unsubstituted alkynyl, e.g., substituted or unsubstituted C₂₋₆alkynyl, substituted or unsubstituted C₂₋₃ alkynyl, substituted or unsubstituted C₃₋₄ alkynyl, substituted or unsubstituted C₄₋₅alkynyl, or substituted or unsubstituted C₅₋₆ alkynyl. Exemplary substituted or unsubstituted R³ alkynyl groups include, but are not limited to, ethynyl, propynyl, or butynyl, unsubstituted or substituted with alkyl, halo, haloalkyl (e.g., CF₃), alkoxyalkyl, cycloalkyl (e.g., cyclopropyl or cyclobutyl), or hydroxyl. In certain embodiments, R³ is selected from the group consisting of trifluoroethynyl, cyclopropylethynyl, cyclobutylethynyl, and propynyl, fluoropropynyl, and chloroethynyl. In certain embodiments, R³ is ethynyl (C₂), propynyl (C₃), or butynyl (C₄), unsubstituted or substituted with one or more substituents selected from the group consisting of substituted or unsubstituted aryl, substituted or unsubstituted heteroaryl, substituted or unsubstituted carbocyclyl, and substituted or unsubstituted heterocyclyl. In certain embodiments, R³ is ethynyl (C₂), propynyl (C₃), or butynyl (C₄) substituted with substituted phenyl. In certain embodiments, the phenyl substituent is further substituted with one or more substituents selected from the group consisting of halo, alkyl, trifluoroalkyl, alkoxy, acyl, amino or amido. In certain embodiments, R³ is ethynyl (C₂), propynyl (C₃), or butynyl (C₄) substituted with substituted or unsubstituted pyrrolyl, imidazolyl, pyrazolyl, oxazoyl, thiazolyl, isoxazoyl, 1,2,3-triazolyl, 1,2,4-triazolyl, oxadiazolyl, thiadiazolyl, or tetrazolyl.

In certain embodiments, R³ is ethynyl, propynyl, or butynyl, unsubstituted or substituted with alkyl, halo, haloalkyl, alkoxyalkyl, or hydroxyl. In certain embodiments, R³ is ethynyl or propynyl, substituted with substituted or unsubstituted aryl. In certain embodiments, R³ is ethynyl or propynyl, substituted with phenyl unsubstituted or substituted with halo, alkyl, alkoxy, haloalkyl, trihaloalkyl, or acyl. In certain embodiments, R³ is ethynyl or propynyl, substituted with substituted or unsubstituted carbocyclyl. In certain embodiments, R³ is ethynyl or propynyl, substituted with substituted or unsubstituted cyclopropyl, cyclobutyl, cyclopentyl, or cyclohexyl. In certain embodiments, R³ is ethynyl or propynyl, substituted with substituted or unsubstituted heteroaryl. In certain embodiments, R³ is ethynyl or propynyl, substituted with substituted or unsubstituted pyridinyl, or pyrimidinyl. In certain embodiments, R³ is ethynyl or propynyl, substituted with substituted or unsubstituted pyrrolyl, imidazolyl, pyrazolyl, oxazoyl, thiazolyl, isoxazoyl, 1,2,3-triazolyl, 1,2,4-triazolyl, oxadiazolyl, thiadiazolyl, tetrazolyl. In certain embodiments, R³ is ethynyl or propynyl, substituted with substituted or unsubstituted heterocyclyl. In certain embodiments, R³ is ethynyl or propynyl, substituted with substituted or unsubstituted pyrrolidinyl, piperidinyl, piperazinyl, or mopholinyl. In certain embodiments, R³ is propynyl or butynyl, substituted with hydroxyl or alkoxy. In certain embodiments, R³ is propynyl or butynyl, substituted with methoxy or ethoxy. In certain embodiments, R³ is ethynyl or propynyl, substituted with chloro. In certain embodiments, R³ is ethynyl or propynyl, substituted with trifluoromethyl.

In certain embodiments, R³ is substituted or unsubstituted carbocyclyl, e.g., substituted or unsubstituted C₃₋₁₀carbocyclyl, substituted or unsubstituted C₃₋₆carbocyclyl, substituted or unsubstituted C₃₋₄ carbocyclyl, substituted or unsubstituted C₄₋₅ carbocyclyl, or substituted or unsubstituted C₅₋₆ carbocyclyl. In certain embodiments, R³ is substituted or unsubstituted cyclopropyl or substituted or unsubstituted cyclobutyl.

### Group R¹¹—as it Relates to Formulae (E-I)-(E-IV-E-i-2b)

In certain embodiments, the bond between C9 and C11 is a double bond, R⁹ is absent and R¹¹ is selected from the group consisting of hydrogen, halogen, substituted or unsubstituted C₁₋₆alkyl, substituted or unsubstituted C₂₋₆alkenyl, substituted or unsubstituted C₂₋₆alkynyl, substituted or unsubstituted C₃₋₁₀carbocyclyl, substituted or unsubstituted C₁₋₆ alkoxy, substituted or unsubstituted 3- to 10-membered heterocyclyl, —OH, and -amino. In certain embodiments, the bond between C9 and C11 is a double bond, R⁹ is absent and R¹¹ is selected from the group consisting of hydrogen, halogen, substituted or unsubstituted C₁₋₆ alkyl, substituted or unsubstituted C₂₋₆alkenyl, substituted or unsubstituted C₂₋₆alkynyl, substituted or unsubstituted C₃₋₁₀carbocyclyl, substituted or unsubstituted C₁₋₆alkoxy, and substituted or unsubstituted 3- to 10-membered heterocyclyl. In certain embodiments, the bond between C9 and C11 is a double bond, R⁹ is absent and R¹¹ is selected from the group consisting of hydrogen, halogen, substituted or unsubstituted C₁₋₆alkyl, substituted or unsubstituted C₂₋₆alkenyl, substituted or unsubstituted C₂₋₆alkynyl, and substituted or unsubstituted C₃₋₁₀carbocyclyl. In certain embodiments, the bond between C9 and C11 is a double bond, Ro is absent and R¹¹ is selected from the group consisting of hydrogen, halogen, substituted or unsubstituted C₁₋₆alkyl, and substituted or unsubstituted C₁₋₆alkoxy. In certain embodiments, the bond between C9 and C11 is a double bond, R⁹ is absent and R¹¹ is selected from the group consisting of hydrogen, halogen, and substituted or unsubstituted C₁₋₆alkyl. In certain embodiments, the bond between C9 and C11 is a double bond, R⁹ is absent and R¹¹ is hydrogen. In certain embodiments, the bond between C9 and C11 is a double bond, R⁹ is absent and R¹¹ is unsubstituted C₁₋₆alkyl.

In certain embodiments, the bond between C9 and C11 is a single bond, R⁹ is hydrogen and R¹¹ is halogen or substituted or unsubstituted C14alkyl. In certain embodiments, the bond between C9 and C11 is a single bond, R⁹ is hydrogen and R¹¹ is an unsubstituted C₁₋₆alkyl.

In certain embodiments, R⁹ and R¹¹ are taken together with the carbon atoms to which they are attached, to form a substituted or unsubstituted C₃₋₆carbocyclyl ring. In certain embodiments, R⁹ and R¹¹ are taken together with the carbon atoms to which they are attached, to form an unsubstituted C₃₋₆carbocyclyl ring. In certain embodiments, R⁹ and R¹¹ are taken together with the carbon atoms to which they are attached, to form a cyclopropyl.

Groups R⁵ and R⁶—as they Relate to Formulae (E-I)-(E-IV-E-i-2b)

In certain embodiments, R⁵ is hydrogen, halogen, or substituted or unsubstituted C₁₋₆ alkyl. In some embodiments, R⁵ is halogen. In some embodiments, R⁵ is substituted C₁₋₆alkyl. In certain embodiments, R⁵ is substituted or unsubstituted alkyl, e.g., substituted or unsubstituted C₁₋₆alkyl, substituted or unsubstituted C₁₋₂ alkyl, substituted or unsubstituted C₂₋₃alkyl, substituted or unsubstituted C₃₋₄ alkyl, substituted or unsubstituted C₄₋₅ alkyl, or substituted or unsubstituted C₅₋₆ alkyl. In some embodiments, R⁵ is substituted or unsubstituted C₁₋₆alkyl. In some embodiments, R⁵ is unsubstituted C₁₋₆alkyl. In some embodiments, R⁵ is unsubstituted C₁ alkyl. In some embodiments, R⁵ is —CH₃. In some embodiments, when the bond between C5 and C6 is a double bond, R⁵ is absent. In some embodiments, the bond between C5 and C6 is a single bond and R⁵ is in the alpha configuration. In some embodiments, the bond between C5 and C6 is a single bond and R⁵ is hydrogen in the beta configuration. In some embodiments, the bond between C5 and C6 is a single bond and R⁵ is hydrogen in the alpha configuration.

In some embodiments, R⁶ is hydrogen, substituted or unsubstituted alkyl, or halogen. In certain embodiments, R⁶ is hydrogen. In certain embodiments, R⁶ is halogen, e.g., fluoro. In certain embodiments, R⁶ is substituted or unsubstituted alkyl, e.g., substituted or unsubstituted C₁₋₆alkyl, substituted or unsubstituted C₁₋₂ alkyl, substituted or unsubstituted C₂₋₃ alkyl, substituted or unsubstituted C₃₋₄ alkyl, substituted or unsubstituted C₄₋₅alkyl, or substituted or unsubstituted C₅₋₆ alkyl. In certain embodiments, R⁶ is C₁ alkyl, e.g., —CH₃ or —CF₃. In certain embodiments, R⁶ is hydrogen, —CH₃, or —F. In certain embodiments, wherein [symbol] represents a single bond, R⁶ is a non-hydrogen substituent in the alpha configuration. In certain embodiments, wherein [symbol] represents a single bond, R⁶ is a non-hydrogen substituent in the beta configuration.

Groups R¹⁸ and R¹⁹—as they Relate to Formulae (E-I)-(E-IV-E-i-2b)

In some embodiments, R¹⁸ is hydrogen, substituted or unsubstituted C₁₋₆alkyl, or OR^{A18}, wherein each instance of R^{A18} is independently substituted or unsubstituted C₁₋₆alkyl. In some embodiments, R¹⁸ is hydrogen, unsubstituted C₂₋₆alkyl, C₁₋₆alkyl substituted with OR^{A18}, or —OR^{A18}, wherein each instance of R^{A18} is independently substituted or unsubstituted C₁₋₆alkyl. In some embodiments, R¹⁸ is hydrogen, unsubstituted C₁₋₆alkyl, C₁₋₆alkyl substituted with OR^{A18}, or —OR^{A18}, wherein each instance of R^{A18} is independently unsubstituted C₁₋₆alkyl. In some embodiments, R¹⁸ is unsubstituted C₁₋₆alkyl. In some embodiments, R¹⁸ is —CH₃, —CH₂CH₃, or —CH(CH₃)₂. In some embodiments, R¹⁸ is —CH₃. In some embodiments, R¹⁸ is —CH₂CH₃, or —CH(CH₃)₂. In some embodiments, R¹⁸ is substituted C₁₋₆alkyl. In some embodiments, R¹⁸ is C₁₋₆alkyl substituted with one or more OR^{A18}, wherein each instance of R^{A18} is independently substituted or unsubstituted C₁₋₆alkyl. In some embodiments, R¹⁸ is C₁₋₆alkyl substituted with OR^{A18}, wherein R^{A18} is substituted or unsubstituted C₁₋₆alkyl. In some embodiments, R¹⁸ is —OR^{A18}, wherein R^{A18} is substituted or unsubstituted C₁₋₆alkyl. In some embodiments, R¹⁸ is hydrogen, —CH₃, —CH₂OCH₃, —OCH₃, —CH₂CH₃, or —CH(CH₃)₂. In some embodiments, R¹⁸ is hydrogen, —CH₂OCH₃, —OCH₃, —CH₂CH₃, or —CH(CH₃)₂. In some embodiments, R¹⁸ is methyl. In some embodiments R¹⁸ is hydrogen.

In some embodiments R¹⁹ is hydrogen, substituted or unsubstituted C₁₋₆alkyl, or OR^{A19}; wherein each instance of R^{A19} is independently substituted or unsubstituted C₁₋₆ alkyl. In some embodiments, R¹⁹ is hydrogen, unsubstituted C₂₋₆alkyl, C₁₋₆alkyl substituted with OR^{A19}, or —OR^{A19}, wherein each instance of R^{A19} is independently substituted or unsubstituted C₁₋₆alkyl. In some embodiments, R¹⁹ is hydrogen, unsubstituted C₁₋₆alkyl, C₁₋₆alkyl substituted with OR^{A19}, or —OR^{A19}, wherein each instance of R^{A19} is independently unsubstituted C₁₋₆alkyl. In some embodiments, R¹⁹ is unsubstituted C₁₋₆alkyl. In some embodiments, R¹⁹ is —CH₃, —CH₂CH₃, or —CH(CH₃)₂. In some embodiments, R¹⁹ is —CH₃. In some embodiments, R¹⁹ is —CH₂CH₃, or —CH(CH₃)₂. In some embodiments, R¹⁹ is substituted C₁₋₆alkyl. In some embodiments, R¹⁹ is C₁₋₆alkyl substituted with one or more OR^{A19}; wherein each instance of R^{A19} is independently substituted or unsubstituted C₁₋₆ alkyl. In some embodiments, R¹⁹ is C₁₋₆alkyl substituted with OR^{A19}, wherein R^{A19} is substituted or unsubstituted C₁₋₆alkyl. In some embodiments, R¹⁹ is —OR^{A19}; wherein R^{A19} is substituted or unsubstituted C₁₋₆alkyl. In some embodiments, R¹⁹ is hydrogen, —CH₃, —CH₂OCH₃, —OCH₃, —CH₂CH₃, or —CH(CH₃)₂. In some embodiments, R¹⁹ is hydrogen, —CH₂OCH₃, —OCH₃, —CH₂CH₃, or —CH(CH₃)₂. In some embodiments, R¹⁹ is hydrogen, —CH₃, or —CH₂CH₃. In some embodiments, R¹⁹ is —CH₃. In some embodiments, R¹⁹ is —CH₂CH₃. In some embodiments R¹⁹ is hydrogen.

In some embodiments R¹⁹ is hydrogen or —CH₃. In some embodiments, R¹⁹ is hydrogen. In some embodiments R¹⁹ is —CH₃.
Groups X, n, R^{23a}, R²³ⁿ, R^{24a}, and R^{24b}—as they Relate to Formulae (E-I)-(E-IV-E-i-2b)

In certain embodiments X is —(C(R^{X})₂)ₙ— or —O—, wherein R^{X} is hydrogen or fluorine, or one R^{X} group and R^{23b} are joined to form a double bond.

In certain embodiments, X is —O—. In certain embodiments, X is —CH₂—. In certain embodiments, X is —CF₂—.

In some embodiments, X is —(C(R^{X})₂—, wherein R^{X} is hydrogen or fluorine, wherein X is —(C(R^{X})₂)ₙ— n is 1 or 2 and R^{X} is hydrogen. In some embodiments, X is —CH₂—. In some embodiments, X is —CH₂—CH₂—.

In certain embodiments, n is 1 or 2. In some embodiments, n is 1. In some embodiments, n is 2.

In some embodiments, each of R^{23a} and R^{23b} is hydrogen. In some embodiments, each of R^{23a} and R^{23b} is fluorine. In certain embodiments, at least one of R^{23a} and R^{23b} is fluorine. In certain embodiments, R^{X} and R^{23b} are joined to form a double bond, e.g., cis or trans double bond.

In some embodiments, R^{24a} is selected from the group consisting of substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl. In some embodiments, R^{24a} is substituted or unsubstituted C₁₋₆alkyl. In some embodiments, R^{24a} is unsubstituted C₁₋₆alkyl. In some embodiments, R^{24a} is C₁₋₆alkyl substituted with fluorine. In some embodiments, R^{24a} is selected from the group consisting of CH₃, —CH(CH₃)₂, or —CH₂CF₃.

In some embodiments, R^{24b} is hydrogen or a substituted or unsubstituted C₁₋₆alkyl. In some embodiments, R^{24b} is hydrogen. In some embodiments, R^{24b} is unsubstituted C₁₋₆alkyl. In some embodiments, R^{24b} is C₁₋₆alkyl substituted with fluorine. In some embodiments, R^{24b} is —CH₃, —CH(CH₃)₂, —CH₂—CF₃.

In some embodiments, R^{24a} and R^{24b}, taken together with the carbon atom to which they are attached, form a substituted or unsubstituted C₃₋₆carbocyclyl, or substituted or unsubstituted 3- to 6-membered heterocyclyl. In some embodiments, R^{24a} and R^{24b}, taken together with the carbon atom to which they are attached, form a substituted or unsubstituted C₃₋₆carbocyclyl or substituted or unsubstituted 3- to 6-membered heterocyclyl. In some embodiments, R^{24a} and R^{24b}, taken together with the carbon atom to which they are attached, form an unsubstituted C₃₋₆carbocyclyl. In some embodiments, R^{24a} and R^{24b}, taken together with the carbon atoms to which they are attached, form a cyclopropyl or cyclopentyl. In some embodiments, R^{24a} and R^{24b}, taken together with the carbon atom to which they are attached, form an unsubstituted 3- to 6-membered heterocyclyl. In some embodiments, R^{24a} and R^{24b}, taken together with the carbon atoms to which they are attached, form a tetrahydrofuranyl.

q, r and s—as they Relate to Formulae (E-I)-(E-IV-E-i-2b)

In some embodiments, q is 0, 1 or 2. In some embodiments, q is 0. In some embodiments, q is 1. In some embodiments, q is 2.

In some embodiments, r is 1 or 2. In some embodiments, r is 1. In some embodiments, r is 2.

In some embodiments, s is 1 or 2. In some embodiments, s is 1. In some embodiments, s is 2.

In some embodiments, q is 0 and r is 1. In some embodiments, q is 2 and r is 1. In some embodiments, q, r and s are each independently 1.

In some embodiments, the compound of Formula (E-I) is any one of Compounds E-1 to E-16, C-26 to C-35 in Table E-1 or pharmaceutically acceptable salts thereof. In some embodiments, the compound of Formula (E-I) is any one of the Compounds E-1 to E-16, or C-26 to C-35 in Table E-1. In some embodiments, the compound of Formula (E-I) is a pharmaceutically acceptable salt of any one of Compounds E-1 to E-16, C-26 to C-35 in Table E-1. In some embodiments, the compound of Formula (E-I) is any one of Compounds E-3, E-4, E-6, E-9, E-11, or E-12 in Table E-1 or pharmaceutically acceptable salts thereof. In some embodiments, the compound of Formula (E-I) is any one of Compounds E-3, E-4, E-6, E-9, E-11, or E-12 in Table E-1. In some embodiments, the compound of Formula (E-I) is a pharmaceutically acceptable salt of any one of Compounds E-3, E-4, E-6, E-9, E-11, or E-12 in Table E-1.

TABLE E-1

| Cmpd | Structure |
|---|---|
| E-1 | 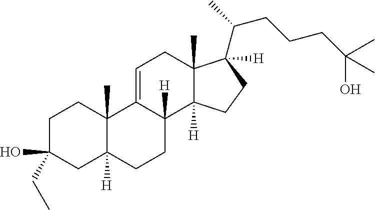 |
| E-2 | 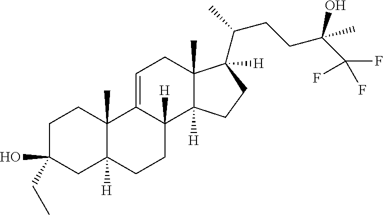 |
| E-3 | 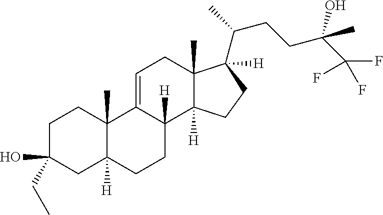 |
| E-4 | 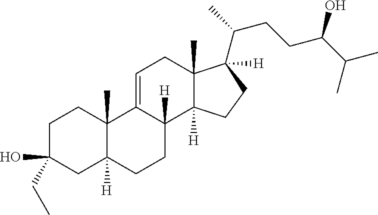 |
| E-5 | 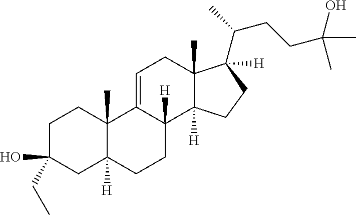 |
| E-6 | 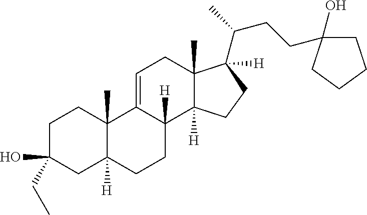 |
| E-7 | 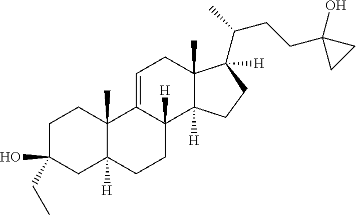 |
| E-8 | 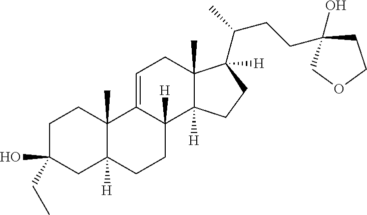 |
| E-9 | 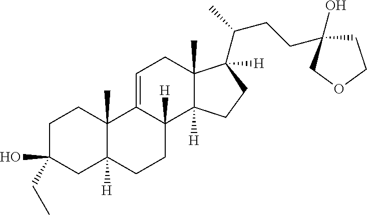 |
| E-10 | 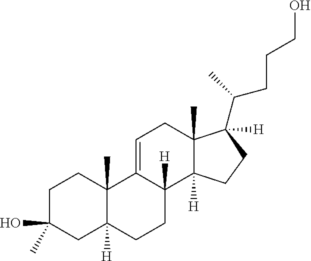 |
| E-11 | 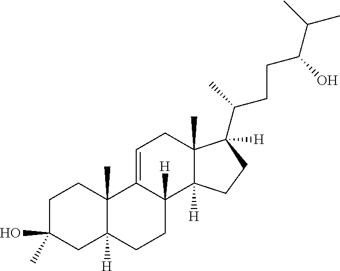 |
| E-12 | 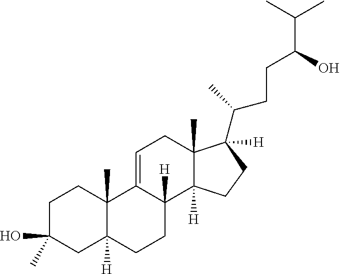 |
| E-13 | 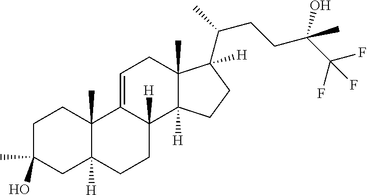 |
| E-14 | 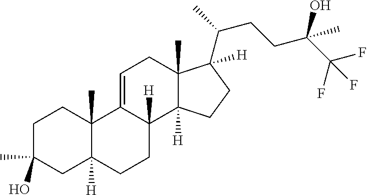 |
| E-15 | 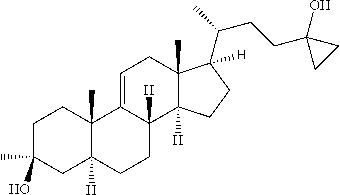 |
| E-16 | 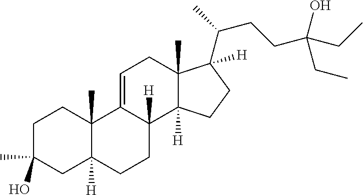 |
| C-26 | 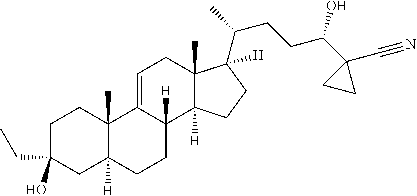 |
| C-27 | 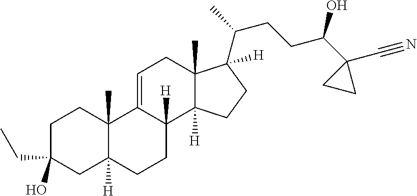 |
| C-28 | 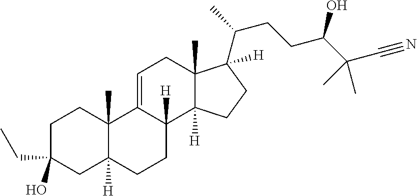 |
| C-29 | 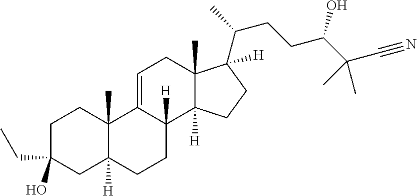 |
| C-30 | 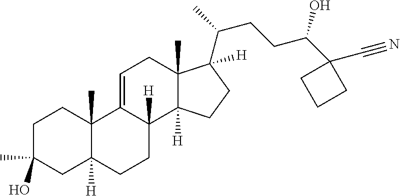 |
| C-31 | 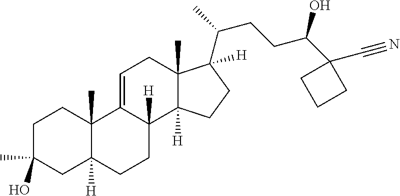 |
| C-32 | 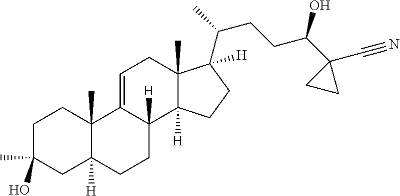 |
| C-33 | 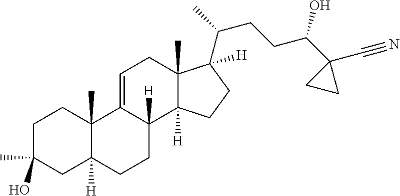 |
| C-34 | 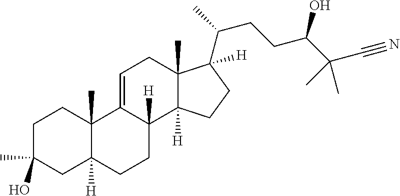 |
| C-35 | 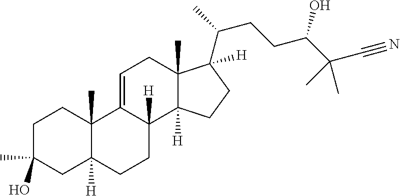 |

In one aspect, the present disclosure provides a compound of Formula (F-I):

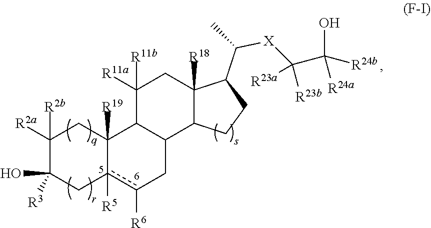

or a pharmaceutically acceptable salt thereof;
wherein:
   each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein R^{A2} is hydrogen or substituted or unsubstituted C₁₋₆alkyl;
   R³ is substituted or unsubstituted C₁₋₆alkyl, substituted or unsubstituted C₂₋₆ alkenyl, substituted or unsubstituted C₂₋₆alkynyl, or substituted or unsubstituted C₃₋₁₀carbocyclyl;
   R⁵ is absent, hydrogen, substituted or unsubstituted C₁₋₆alkyl, or halogen;
   R⁶ is hydrogen, substituted or unsubstituted C₁₋₆alkyl, or halogen;
   R^{11a} is hydrogen or —OR^{A11}, wherein R^{A11} is hydrogen or substituted or unsubstituted C₁₋₆alkyl, and R^{11b} is hydrogen; or R^{11a} and R^{11b} are joined to form an oxo (═O) group;
   R¹⁸ is hydrogen, unsubstituted C₂₋₆alkyl, substituted C₁₋₆alkyl, or OR^{A18}; wherein each instance of R^{A18} is independently substituted or unsubstituted C₁₋₆alkyl;
R¹⁹ is hydrogen, substituted or unsubstituted C₁₋₆alkyl, or OR^{A19}, each instance of R^{A19} is independently substituted or unsubstituted C₁₋₆alkyl;
X is —(C(R^{X})₂)ₙ— or —O—, wherein R^{X} is hydrogen or fluorine, or one R^{X} group and R^{23b} are joined to form a double bond;
   n is 1, 2 or 3;
   each of R^{23a} and R^{23b} is independently hydrogen or fluorine;
   R^{24a} is selected from the group consisting of substituted or unsubstituted C₁₋₆alkyl, substituted or unsubstituted C₂₋₆alkenyl, substituted or unsubstituted C₂₋₆ alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl; and
   R^{24b} is hydrogen or substituted or unsubstituted C₁₋₆alkyl;
   or R^{24a} and R^{24b}, taken together with the carbon atom to which they are attached, form a substituted or unsubstituted C₃₋₆carbocyclyl, or a substituted or unsubstituted 3- to 10-membered heterocyclyl;
   q is 0, 1 or 2;
   r is 1 or 2;
   s is 1 or 2; and
   [symbol] represents a single or double bond, provided if a double bond is present, R⁵ is absent.

In one aspect, the compound of Formula (F-I) is a compound of Formula (F-IA):

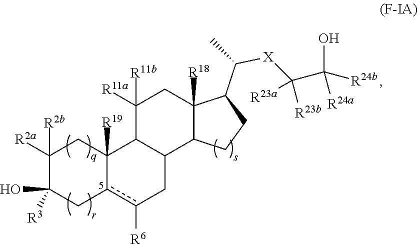

or a pharmaceutically acceptable salt thereof;
wherein:
   each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein R^{A2} is hydrogen or substituted or unsubstituted C₁₋₆alkyl;
   R³ is substituted or unsubstituted C₁₋₆alkyl, substituted or unsubstituted C₂₋₆alkenyl, substituted or unsubstituted C₂₋₆alkynyl, or substituted or unsubstituted C₃₋₁₀carbocyclyl;
   R⁶ is hydrogen, substituted or unsubstituted C₁₋₆alkyl, or halogen;
   R^{11a} is hydrogen or —OR^{A11}, wherein R^{A11} is hydrogen or substituted or unsubstituted C₁₋₆alkyl, and R^{11b} is hydrogen; or R^{11a} and R^{11b} are joined to form an oxo (═O) group;
   R¹⁸ is hydrogen, unsubstituted C₂₋₆alkyl, substituted C₁₋₆alkyl, or OR^{A18}, wherein each instance of R^{A18} is independently substituted or unsubstituted C₁₋₆alkyl;
      R¹⁹ is hydrogen, substituted or unsubstituted C₁₋₆alkyl, or OR^{A19}, each instance of R^{A19} is independently substituted or unsubstituted C₁₋₆alkyl;
      X is —(C(R^{X})₂)ₙ— or —O—, wherein R^{X} is hydrogen or fluorine, or one R^{X} group and R^{23b} are joined to form a double bond;
   n is 1, 2 or 3;
   each of R^{23a} and R^{23b} is independently hydrogen or fluorine;
   R^{24a} is selected from the group consisting of substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl; and
   R^{24b} is hydrogen or substituted or unsubstituted C₁₋₆alkyl;
   or R^{24a} and R^{24b}, taken together with the carbon atom to which they are attached, form a substituted or unsubstituted C₃₋₆carbocyclyl, or a substituted or unsubstituted 3- to 10-membered heterocyclyl;
   q is 0, 1 or 2;
   r is 1 or 2;
   s is 1 or 2; and
   [symbol] represents a single or double bond, provided if a single bond is present, then the hydrogen at C5 is in the alpha configuration.

In some embodiments, the compound of Formula (F-I) is a compound of Formula (F-Ia):

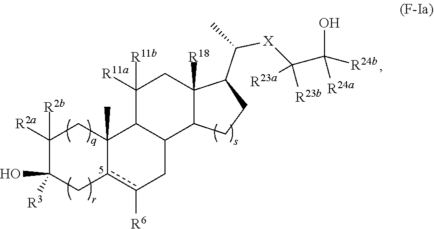

or a pharmaceutically acceptable salt thereof.

In some embodiments, the compound of Formula (F-Ia) is a compound of Formula (F-Ia-i):

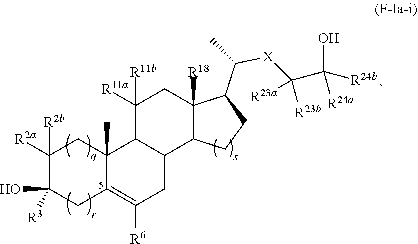

or a pharmaceutically acceptable salt thereof.

In some embodiments, the compound of Formula (F-Ia-i) is a compound of Formula (F-Ia-i-1) or Formula (F-Ia-i-2):

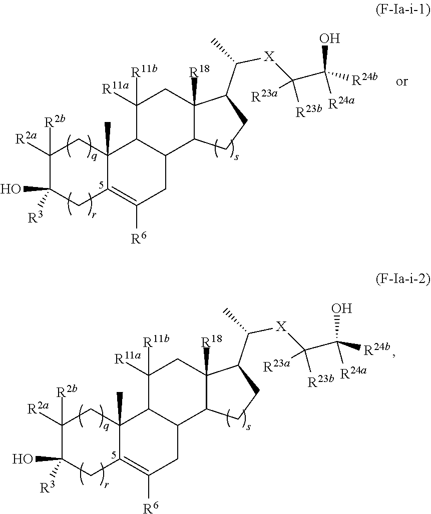

or a pharmaceutically acceptable salt thereof.

In some embodiments, the compound of Formula (F-Ia) is a compound of Formula (F-Ia-ii):

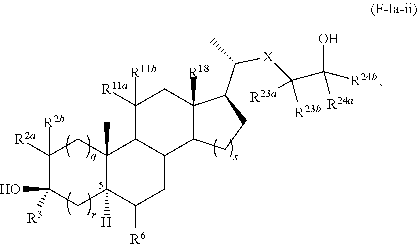

or a pharmaceutically acceptable salt thereof.

In some embodiments, the compound of Formula (F-Ia-ii) is a compound of Formula (F-Ia-ii-1) or Formula (F-Ia-ii-2):

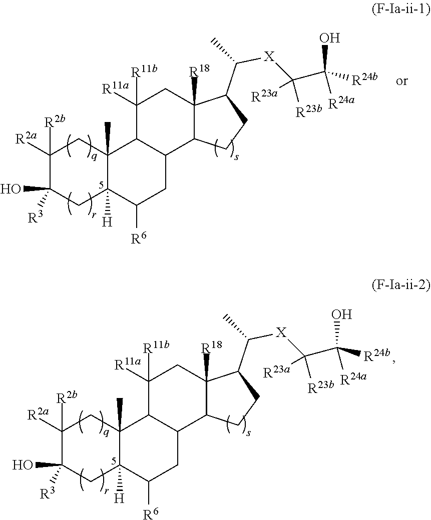

or a pharmaceutically acceptable salt thereof.

In some embodiments, the compound of Formula (F-I) is a compound of Formula (F-Ib):

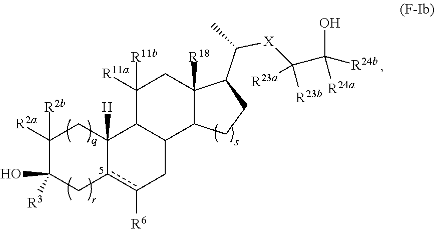

or a pharmaceutically acceptable salt thereof.

In some embodiments, the compound of Formula (F-Ib) is a compound of Formula (F-Ib-i):

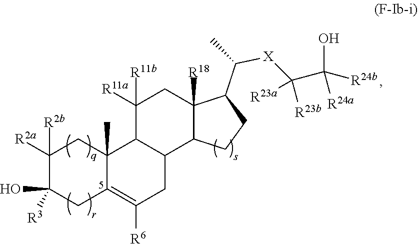

or a pharmaceutically acceptable salt thereof.

In some embodiments, the compound of Formula (F-Ib-i) is a compound of Formula (F-Ib-i-1) or Formula (F-Ib-i-2):

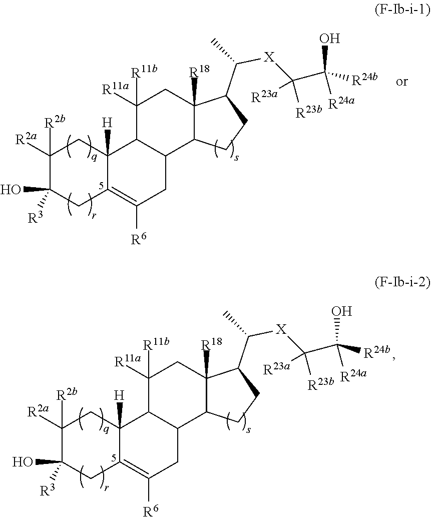

or a pharmaceutically acceptable salt thereof.

In some embodiments, the compound of Formula (F-Ib) is a compound of Formula (F-Ib-ii):

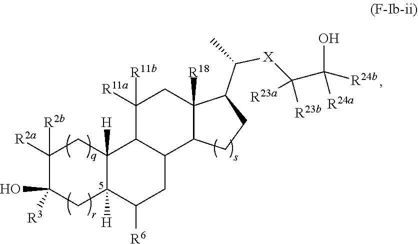

or a pharmaceutically acceptable salt thereof.

In some embodiments, the compound of Formula (F-Ib-ii) is a compound of Formula (F-Ib-ii-1) or Formula (F-Ib-ii-2):

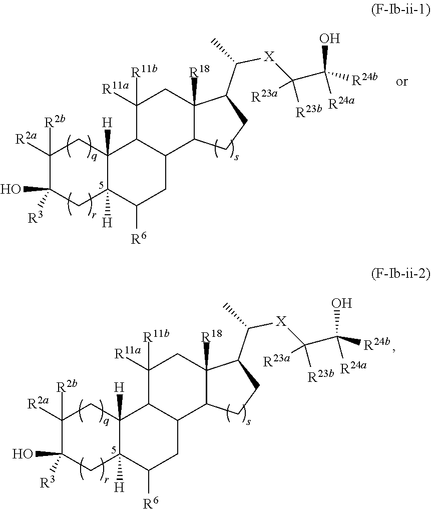

or a pharmaceutically acceptable salt thereof.

In some embodiments, the compound of Formula (F-I) is a compound of Formula (F-II):

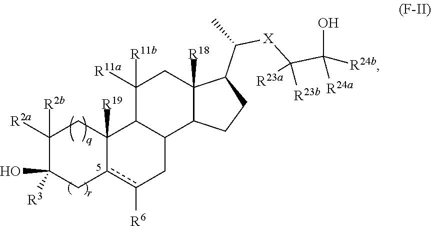

or a pharmaceutically acceptable salt thereof.

In some embodiments, the compound of Formula (F-II) is a compound of Formula (F-IIa):

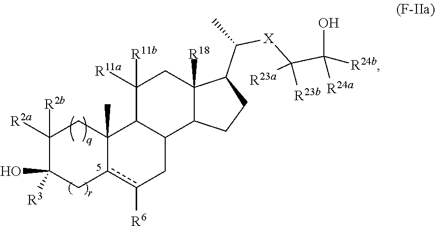

or a pharmaceutically acceptable salt thereof.

In some embodiments, the compound of Formula (F-IIa) is a compound of Formula (F-IIa-i):

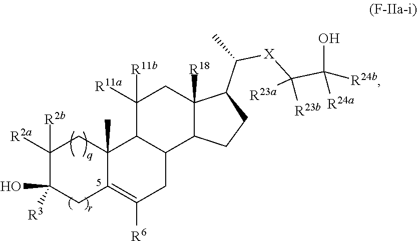

or a pharmaceutically acceptable salt thereof.

In some embodiments, the compound of Formula (F-IIa-i) is a compound of Formula (F-IIa-i-1) or Formula (F-IIa-i-2):

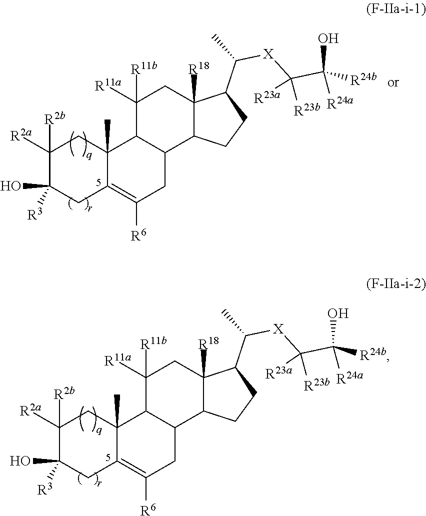

or a pharmaceutically acceptable salt thereof.

In some embodiments, the compound of Formula (F-IIa) is a compound of Formula (F-IIa-ii):

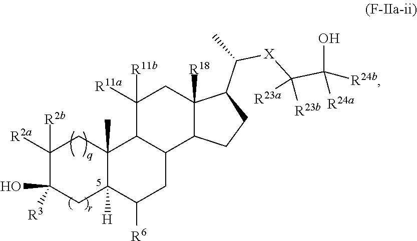

or a pharmaceutically acceptable salt thereof.

In some embodiments, the compound of Formula (F-IIa-ii) is a compound of Formula (F-IIa-ii-1) or Formula (F-IIa-ii-2):

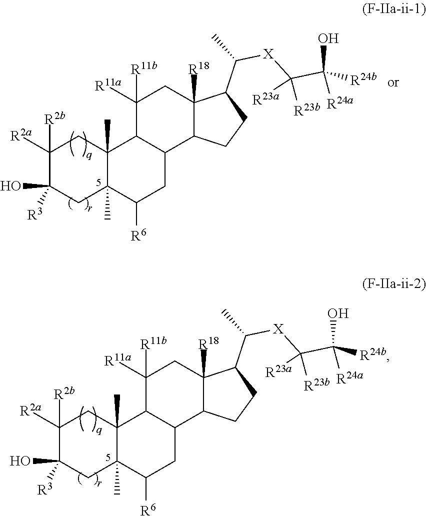

or a pharmaceutically acceptable salt thereof.

In some embodiments, the compound of Formula (F-II) is a compound of Formula (F-IIb):

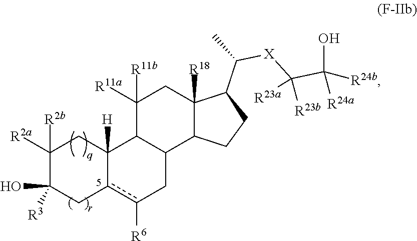

or a pharmaceutically acceptable salt thereof.

In some embodiments, the compound of Formula (F-IIb) is a compound of Formula (F-IIb-i):

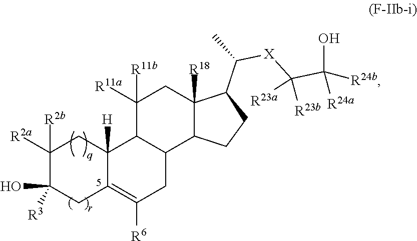

or a pharmaceutically acceptable salt thereof.

In some embodiments, the compound of Formula (F-IIb-i) is a compound of Formula (F-IIb-i-1) or Formula (F-IIb-i-2):

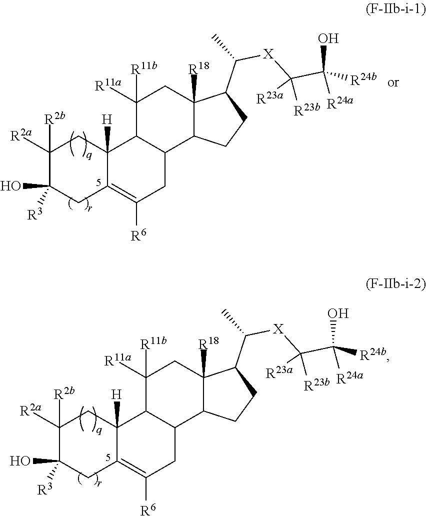

or a pharmaceutically acceptable salt thereof.

In some embodiments, the compound of Formula (F-IIb) is a compound of Formula (F-IIb-ii):

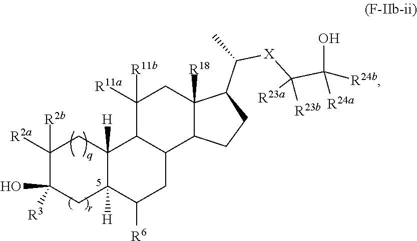

or a pharmaceutically acceptable salt thereof.

In some embodiments, the compound of Formula (F-IIb-ii) is a compound of Formula (F-IIb-ii-1) or Formula (F-IIb-ii-2):

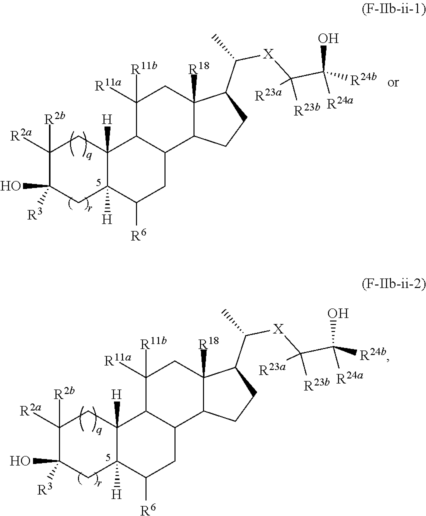

or a pharmaceutically acceptable salt thereof.

In some embodiments, the compound of Formula (F-I) is a compound of Formula (F-III):

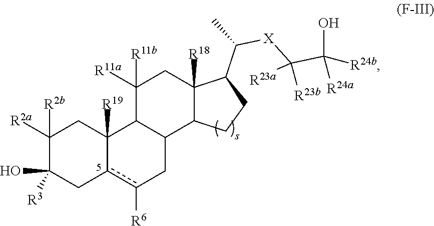

or a pharmaceutically acceptable salt thereof.

In some embodiments, the compound of Formula (F-III) is a compound of Formula (F-IIIa):

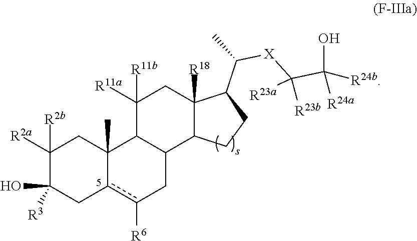

In some embodiments, the compound of Formula (F-IIIa) is a compound of Formula (F-IIIa-i):

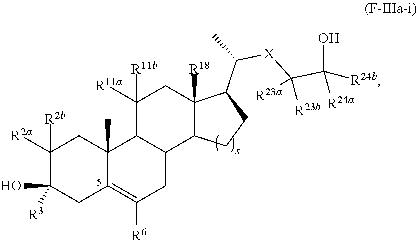

or a pharmaceutically acceptable salt thereof.

In some embodiments, the compound of Formula (F-IIIa-i) is a compound of Formula (F-IIIa-i-1) or Formula (F-IIIa-i-2):

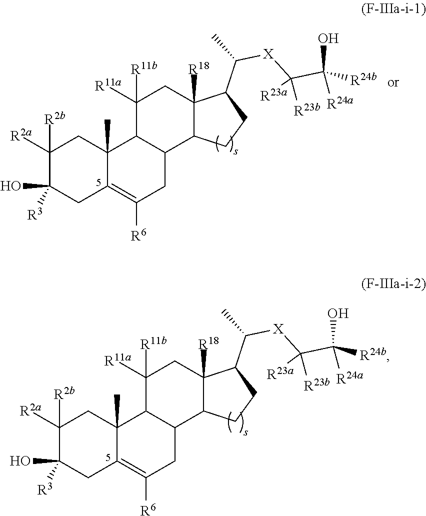

or a pharmaceutically acceptable salt thereof.

In some embodiments, the compound of Formula (F-IIIa) is a compound of Formula (F-IIIa-ii):

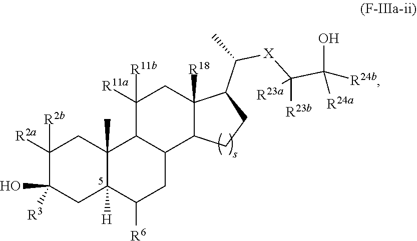

or a pharmaceutically acceptable salt thereof.

In some embodiments, the compound of Formula (F-IIIa-ii) is a compound of Formula (F-IIIa-ii-1) or Formula (F-IIIa-ii-2):

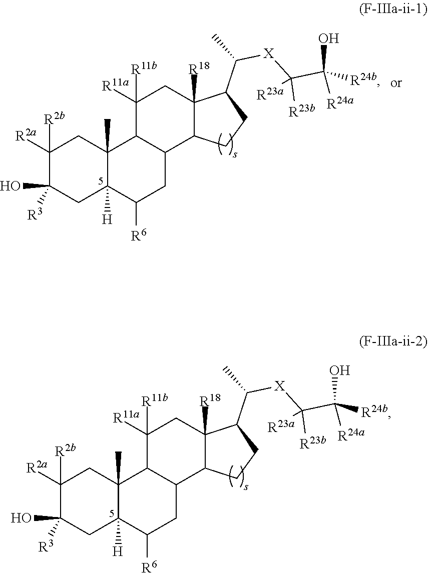

or a pharmaceutically acceptable salt thereof.

In some embodiments, the compound of Formula (F-III) is a compound of Formula (F-IIIb):

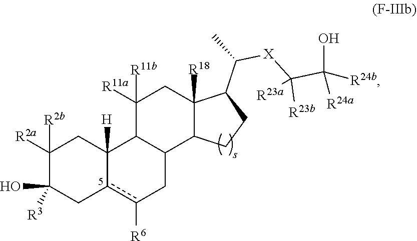

or a pharmaceutically acceptable salt thereof.

In some embodiments, the compound of Formula (F-IIIb) is a compound of Formula (F-IIIb-i):

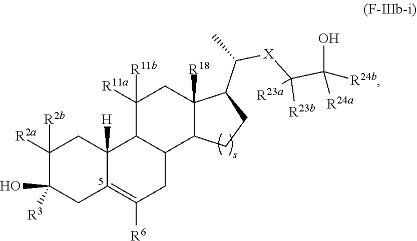

or a pharmaceutically acceptable salt thereof.

In some embodiments, the compound of Formula (F-IIIb-i) is a compound of Formula (F-IIIb-i-1) or Formula (F-IIIb-i-2):

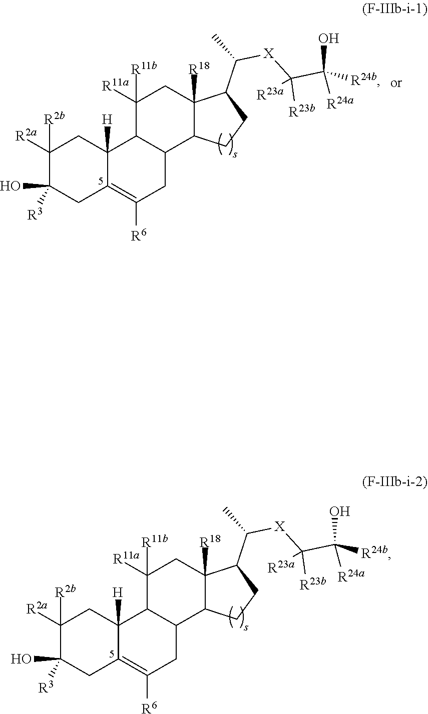

or a pharmaceutically acceptable salt thereof.

In some embodiments, the compound of Formula (F-IIIb) is a compound of Formula (F-IIIb-i):

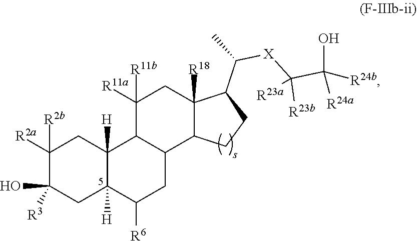

or a pharmaceutically acceptable salt thereof.

In some embodiments, the compound of Formula (F-IIIb-ii) is a compound of Formula (F-IIIb-ii-1) or Formula (F-IIIb-ii-2):

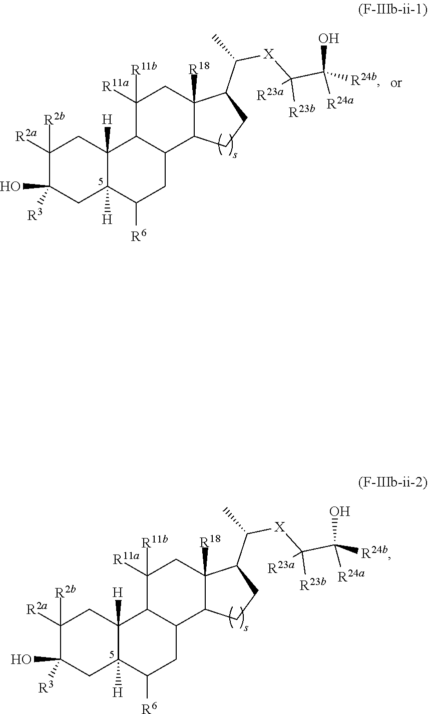

or a pharmaceutically acceptable salt thereof.

In some embodiments, the compound of Formula (F-I) is a compound of Formula (F-IV):

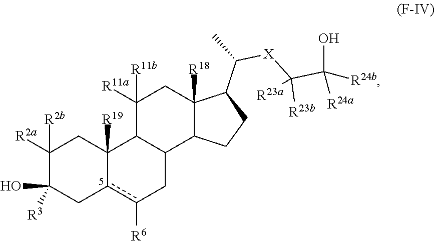

or a pharmaceutically acceptable salt thereof.

In some embodiments, the compound of Formula (F-IV) is a compound of Formula (F-IVa):

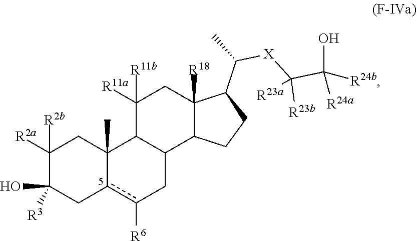

or a pharmaceutically acceptable salt thereof.

In some embodiments, the compound of Formula (F-IVa) is a compound of Formula (F-IVa-i):

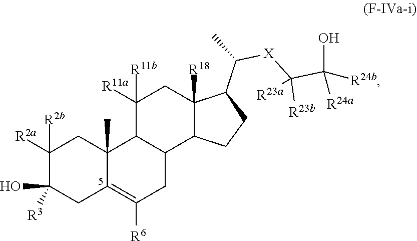

or a pharmaceutically acceptable salt thereof.

In some embodiments, the compound of Formula (F-IVa-i) is a compound of Formula (F-IVa-i-1) or Formula (F-IVa-i-2):

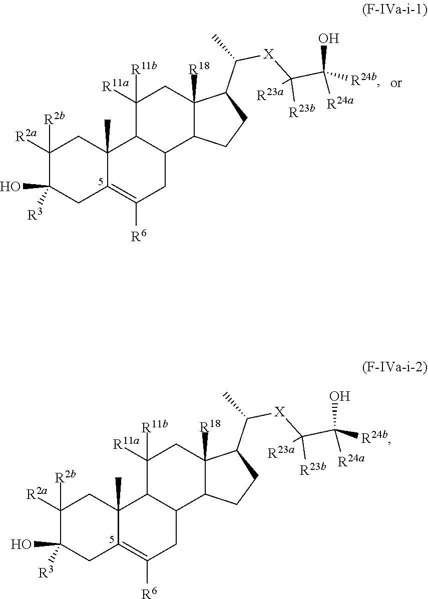

or a pharmaceutically acceptable salt thereof.

In some embodiments, the compound of Formula (F-IVa) is a compound of Formula (F-IVa-ii):

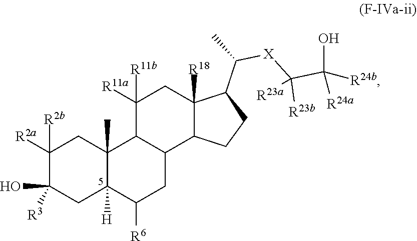

or a pharmaceutically acceptable salt thereof.

In some embodiments, the compound of Formula (F-IVa-ii) is a compound of Formula (F-IVa-ii-1) or Formula (F-IVa-ii-2):

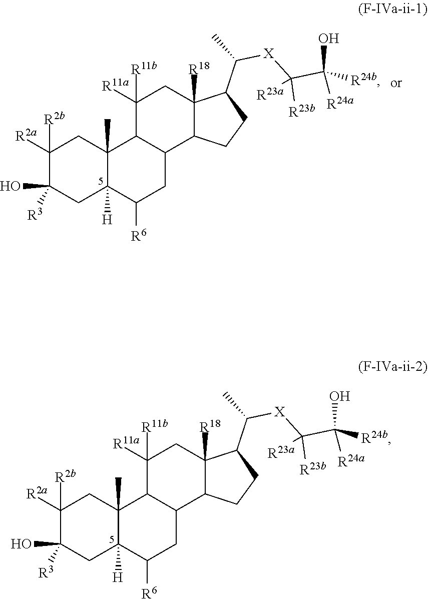

or a pharmaceutically acceptable salt thereof.

In some embodiments, the compound of Formula (F-IV) is a compound of Formula (F-IVb):

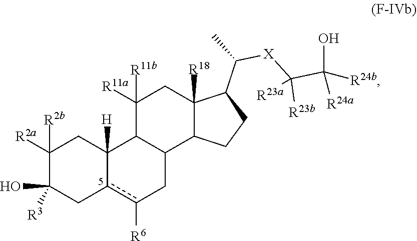

or a pharmaceutically acceptable salt thereof.

In some embodiments, the compound of Formula (F-IVb) is a compound of Formula (F-IVb-i):

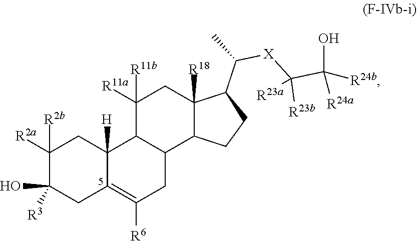

or a pharmaceutically acceptable salt thereof.

In some embodiments, the compound of Formula (F-IVb-i) is a compound of Formula (F-IVb-i-1) or Formula (F-IVb-i-2):

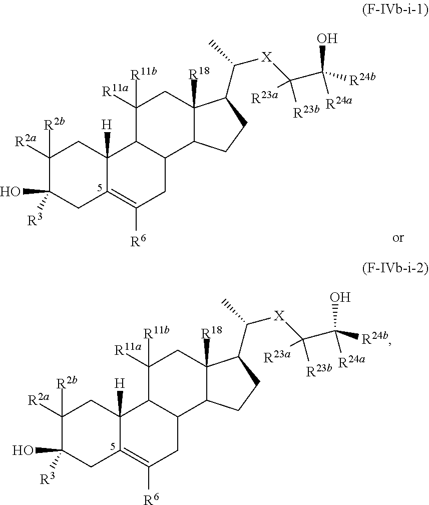

or a pharmaceutically acceptable salt thereof.

In some embodiments, the compound of Formula (F-IVb) is a compound of Formula (F-IVb-ii):

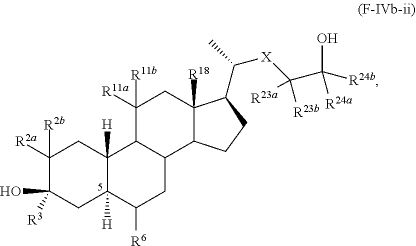

or a pharmaceutically acceptable salt thereof.

In some embodiments, the compound of Formula (F-IVb-ii) is a compound of Formula (F-IVb-ii-1) or Formula (F-IVb-ii-2):

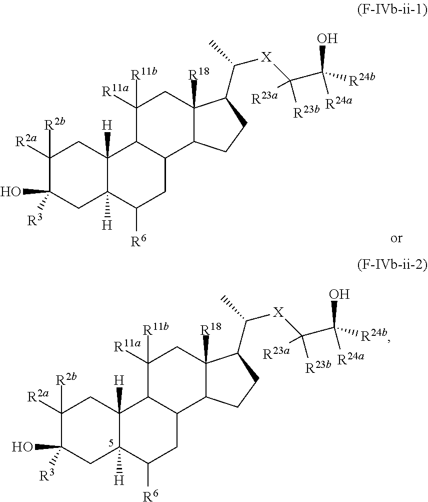

or a pharmaceutically acceptable salt thereof.

In some embodiments, the compound of Formula (F-I) is a compound of Formula (F-V):

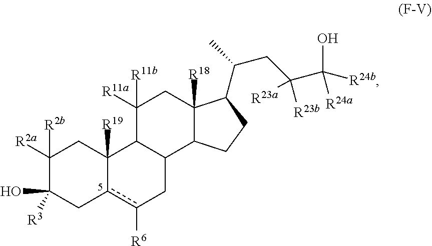

or a pharmaceutically acceptable salt thereof.

In some embodiments, the compound of Formula (F-V) is a compound of Formula (F-Va):

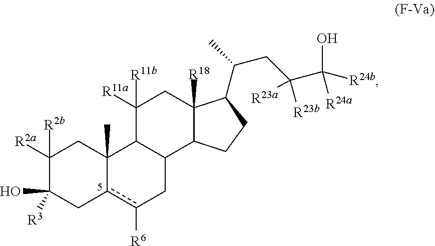

or a pharmaceutically acceptable salt thereof.

In some embodiments, the compound of Formula (F-Va) is a compound of Formula (F-Va-i):

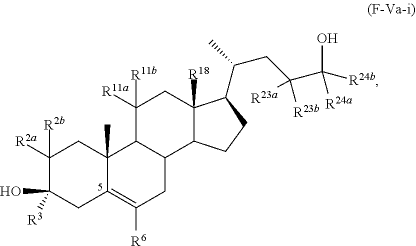

or a pharmaceutically acceptable salt thereof.

In some embodiments, the compound of Formula (F-Va-i) is a compound of Formula (F-Va-i-1) or Formula (F-Va-i-2):

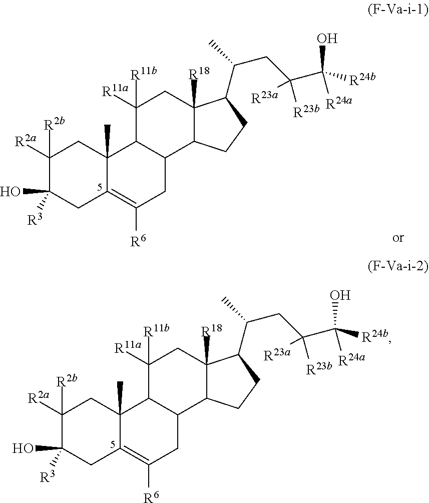

or a pharmaceutically acceptable salt thereof.

In some embodiments, the compound of Formula (F-Va) is a compound of Formula (F-Va-ii):

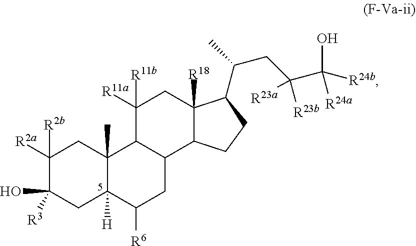

or a pharmaceutically acceptable salt thereof.

In some embodiments, the compound of Formula (F-Va-ii) is a compound of Formula (F-Va-ii-1) or Formula (F-Va-ii-2):

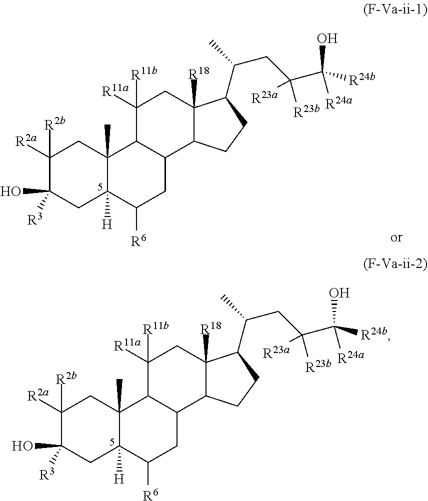

or a pharmaceutically acceptable salt thereof.

In some embodiments, the compound of Formula (F-V) is a compound of Formula (F-Vb):

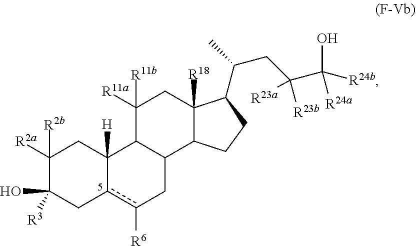

or a pharmaceutically acceptable salt thereof.

In some embodiments, the compound of Formula (F-Vb) is a compound of Formula (F-Vb-i):

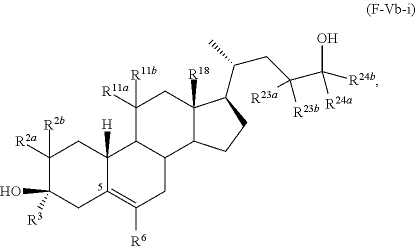

or a pharmaceutically acceptable salt thereof.

In some embodiments, the compound of Formula (F-Vb-i) is a compound of Formula (F-Vb-i-1) or Formula (F-Vb-i-2):

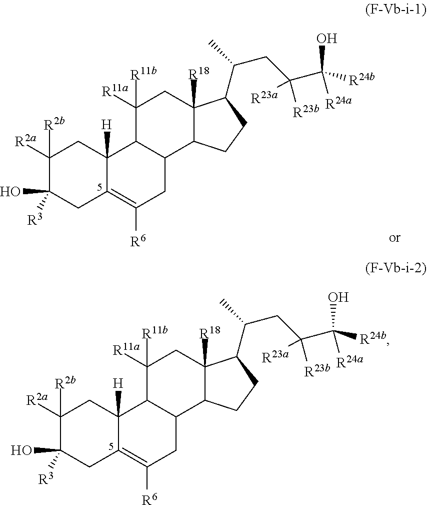

or a pharmaceutically acceptable salt thereof.

In some embodiments, the compound of Formula (F-Vb) is a compound of Formula (F-Vb-ii):

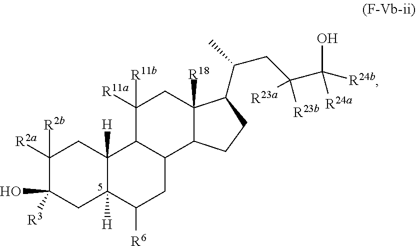

or a pharmaceutically acceptable salt thereof.

In some embodiments, the compound of Formula (F-Vb-ii) is a compound of Formula (F-Vb-ii-1) or Formula (F-Vb-ii-2):

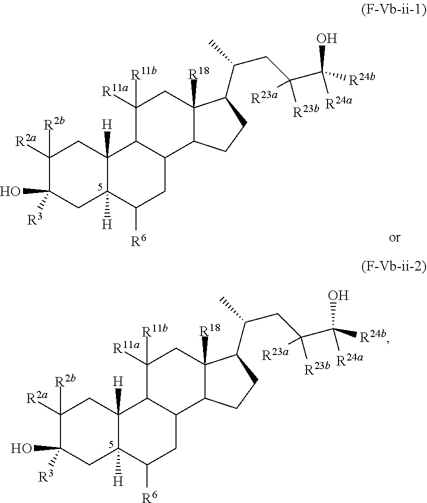

or a pharmaceutically acceptable salt thereof.
   Groups R^{2a} and R^{2b}—as they Relate to Formulae (F-I)-(F-Vb-ii-2)

In certain embodiments, each of R^{2a} and R^{2b} is independently hydrogen, halogen, substituted or unsubstituted C₁₋₆alkyl, substituted or unsubstituted C₃₋₁₀carbocyclyl, or OR^{A2}, wherein R^{A2} is hydrogen or substituted or unsubstituted C₁₋₆alkyl. In certain embodiments, each of R^{2a} and R^{2b} is independently hydrogen, halogen, substituted or unsubstituted C₁₋₆alkyl, substituted or unsubstituted cyclopropyl, or —OR^{A2}, wherein R^{A2} is hydrogen or substituted or unsubstituted C₁₋₆alkyl. In certain embodiments, each of R^{2a} and R^{2b} is hydrogen. In certain embodiments, each of R^{2a} and R^{2b} is independently halogen, e.g., fluoro, chloro, bromo, or iodo. In certain embodiments, each of R^{2a} and R^{2b} is independently fluoro or chloro. In certain embodiments, each of R^{2a} and R^{2b} is independently is substituted or unsubstituted C₁₋₆alkyl, e.g., substituted or unsubstituted C₁₋₂ alkyl, substituted or unsubstituted C₂₋₃ alkyl, substituted or unsubstituted C₃₋₄ alkyl, substituted or unsubstituted C₄₋₅alkyl, or substituted or unsubstituted C₅₋₆ alkyl. In certain embodiments, each of R^{2a} and R^{2b} is independently —CH₃, —CH₂CH₃, —CH₂CH₂CH₃, or cyclopropyl. In certain embodiments, each of R^{2a} and R^{2b} is independently —OR^{A2}. In certain embodiments, R^{A2} is hydrogen. In certain embodiments, R^{A2} is substituted or unsubstituted alkyl, e.g., substituted or unsubstituted C₁₋₆alkyl, substituted or unsubstituted C₁₋₂ alkyl, substituted or unsubstituted C₂₋₃ alkyl, substituted or unsubstituted C₃₋₄ alkyl, substituted or unsubstituted C₄₋₅ alkyl, or substituted or unsubstituted C₅₋₆ alkyl. In certain embodiments, R^{A2} is hydrogen, —CH₃, —CH₂CH₃, or —CH₂CH₂CH₃, i.e., to provide a group of R^{2a} or R^{2b} each independently of formula-OH, —OCH₃, —OCH₂CH₃, or —OCH₂CH₂CH₃. In certain embodiments, each of R^{2a} and R^{2b} is a non-hydrogen substituent. In certain embodiments, one of R^{2a} and R^{2b} is a non-hydrogen substituent in the alpha configuration. In certain embodiments, one of R^{2a} and R^{2b} is a non-hydrogen substituent in the beta configuration.

In some embodiments, each of R^{2a} and R^{2b} is hydrogen.

### Group R³—as it Relates to Formulae (F-I)-(F-Vb-ii-2)

In certain embodiments, R³ is substituted or unsubstituted aliphatic, i.e., substituted or unsubstituted alkyl, substituted or unsubstituted alkenyl, substituted or unsubstituted alkynyl, substituted or unsubstituted carbocyclyl, or substituted or unsubstituted C₃₋₁₀, carbocyclyl.

In certain embodiments, R³ is substituted or unsubstituted alkyl, e.g., substituted or unsubstituted C₁₋₆alkyl, substituted or unsubstituted C₁₋₂ alkyl, substituted or unsubstituted C₂₋₃ alkyl, substituted or unsubstituted C₃₋₄ alkyl, substituted or unsubstituted C₄₋₅ alkyl, or substituted or unsubstituted C₅₋₆ alkyl. Exemplary R³ C₁₋₆alkyl groups include, but are not limited to, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), n-hexyl (C₆), C₁₋₆ alkyl substituted with 1, 2, 3, 4, 5, 6, 7, 8, 9, 10, or more fluoro groups (e.g., —CF₃, —CH₂F, —CHF₂, difluoroethyl, and 2,2,2-trifluoro-1,1-dimethyl-ethyl), C₁₋₆alkyl substituted with 1, 2, 3, 4, 5, 6, 7, 8, 9, 10, or more chloro groups (e.g., —CH₂Cl, —CHCl₂), and C₁₋₆alkyl substituted with 1, 2, 3, 4, 5, 6, 7, 8, 9, 10, or more alkoxy groups (e.g., —CH₂OCH₃, —CH₂OCH₂CH₃, —CH₂O-cyclopropyl). In certain embodiments, R³ is substituted alkyl, e.g., R³ is haloalkyl, alkoxyalkyl, or aminoalkyl. In certain embodiments, R³ is Me, Et, n-Pr, n-Bu, i-Bu, fluoromethyl, chloromethyl, difluoromethyl, trifluoromethyl, trifluoroethyl, difluoroethyl, 2,2,2-trifluoro-1,1-dimethyl-ethyl, methoxymethyl, methoxyethyl, or ethoxymethyl.

In certain embodiments, R³ is unsubstituted C₁₋₆alkyl. In some embodiments, R³ is selected from the group consisting of CH₃, —CH₂CH₃, or —CH₂CH₂CH₃. In some embodiments, R³ is —CH₂CH₃. In some embodiments, R³ is —CH₃.

In certain embodiments, R³ is alkyl substituted with one or more fluorine atoms; e.g., R³ is —CH₂F, —CHF₂, or —CF₃.

In certain embodiments, R³ is alkyl substituted with one or more —OR^{A3} groups, wherein R^{A3} is hydrogen or substituted or unsubstituted alkyl. In certain embodiments, R³ is CH₂OR^{A3}, e.g., wherein R^{A3} is hydrogen, —CH₃, —CH₂CH₃, or —CH₂CH₂CH₃.

In certain embodiments, R³ is substituted or unsubstituted alkenyl, e.g., substituted or unsubstituted C₂₋₆alkenyl, substituted or unsubstituted C₂₋₃alkenyl, substituted or unsubstituted C₃₋₄ alkenyl, substituted or unsubstituted C₄₋₅alkenyl, or substituted or unsubstituted C₅₋₆ alkenyl. In certain embodiments, R³ is ethenyl (C₂), propenyl (C₃), or butenyl (C₄), unsubstituted or substituted with one or more substituents selected from the group consisting of alkyl, halo, haloalkyl, alkoxyalkyl, or hydroxyl. In certain embodiments, R³ is ethenyl, propenyl, or butenyl, unsubstituted or substituted with alkyl, halo, haloalkyl, alkoxyalkyl, or hydroxy. In certain embodiments, R³ is ethenyl.

In certain embodiments, R³ is substituted or unsubstituted alkynyl, e.g., substituted or unsubstituted C₂₋₆alkynyl, substituted or unsubstituted C₂₋₃ alkynyl, substituted or unsubstituted C₃₋₄ alkynyl, substituted or unsubstituted C₄₋₅ alkynyl, or substituted or unsubstituted C₅₋₆ alkynyl. Exemplary substituted or unsubstituted R³ alkynyl groups include, but are not limited to, ethynyl, propynyl, or butynyl, unsubstituted or substituted with alkyl, halo, haloalkyl (e.g., CF₃), alkoxyalkyl, cycloalkyl (e.g., cyclopropyl or cyclobutyl), or hydroxyl. In certain embodiments, R³ is selected from the group consisting of trifluoroethynyl, cyclopropylethynyl, cyclobutylethynyl, and propynyl, fluoropropynyl, and chloroethynyl. In certain embodiments, R³ is ethynyl (C₂), propynyl (C₃), or butynyl (C₄), unsubstituted or substituted with one or more substituents selected from the group consisting of substituted or unsubstituted aryl, substituted or unsubstituted heteroaryl, substituted or unsubstituted carbocyclyl, and substituted or unsubstituted heterocyclyl. In certain embodiments, R³ is ethynyl (C₂), propynyl (C₃), or butynyl (C₄) substituted with substituted phenyl. In certain embodiments, the phenyl substituent is further substituted with one or more substituents selected from the group consisting of halo, alkyl, trifluoroalkyl, alkoxy, acyl, amino or amido. In certain embodiments, R³ is ethynyl (C₂), propynyl (C₃), or butynyl (C₄) substituted with substituted or unsubstituted pyrrolyl, imidazolyl, pyrazolyl, oxazoyl, thiazolyl, isoxazoyl, 1,2,3-triazolyl, 1,2,4-triazolyl, oxadiazolyl, thiadiazolyl, or tetrazolyl.

In certain embodiments, R³ is ethynyl, propynyl, or butynyl, unsubstituted or substituted with alkyl, halo, haloalkyl, alkoxyalkyl, or hydroxyl. In certain embodiments, R³ is ethynyl or propynyl, substituted with substituted or unsubstituted aryl. In certain embodiments, R³ is ethynyl or propynyl, substituted with phenyl unsubstituted or substituted with halo, alkyl, alkoxy, haloalkyl, trihaloalkyl, or acyl. In certain embodiments, R³ is ethynyl or propynyl, substituted with substituted or unsubstituted carbocyclyl. In certain embodiments, R³ is ethynyl or propynyl, substituted with substituted or unsubstituted cyclopropyl, cyclobutyl, cyclopentyl, or cyclohexyl. In certain embodiments, R³ is ethynyl or propynyl, substituted with substituted or unsubstituted heteroaryl. In certain embodiments, R³ is ethynyl or propynyl, substituted with substituted or unsubstituted pyridinyl, or pyrimidinyl. In certain embodiments, R³ is ethynyl or propynyl, substituted with substituted or unsubstituted pyrrolyl, imidazolyl, pyrazolyl, oxazoyl, thiazolyl, isoxazoyl, 1,2,3-triazolyl, 1,2,4-triazolyl, oxadiazolyl, thiadiazolyl, tetrazolyl. In certain embodiments, R³ is ethynyl or propynyl, substituted with substituted or unsubstituted heterocyclyl. In certain embodiments, R³ is ethynyl or propynyl, substituted with substituted or unsubstituted pyrrolidinyl, piperidinyl, piperazinyl, or mopholinyl. In certain embodiments, R³ is propynyl or butynyl, substituted with hydroxyl or alkoxy. In certain embodiments, R³ is propynyl or butynyl, substituted with methoxy or ethoxy. In certain embodiments, R³ is ethynyl or propynyl, substituted with chloro. In certain embodiments, R³ is ethynyl or propynyl, substituted with trifluoromethyl.

In certain embodiments, R³ is substituted or unsubstituted carbocyclyl, e.g., substituted or unsubstituted C₃₋₁₀carbocyclyl, substituted or unsubstituted C₃₋₆carbocyclyl, substituted or unsubstituted C₃₋₄ carbocyclyl, substituted or unsubstituted C₄₋₅ carbocyclyl, or substituted or unsubstituted C₅₋₆ carbocyclyl. In certain embodiments, R³ is substituted or unsubstituted cyclopropyl or substituted or unsubstituted cyclobutyl.

Groups R^{11a} and R^{11b}—as they Relate to Formulae (F-1)-(F-Vb-ii-2)

In certain embodiments, R^{11a} is hydrogen or —OR^{A11}, wherein R^{A11} is hydrogen or substituted or unsubstituted C₁₋₆alkyl, and R^{11b} is hydrogen; or R^{11a} and R^{11b} are joined to form an oxo (═O) group.

In certain embodiments, R^{11a} and R^{11b} are joined to form an oxo (═O) group.

In certain embodiments, R^{11a} is —OR^{A11} and R^{11b} is hydrogen. In certain embodiments, R^{11a} is —OR^{A11} in the alpha or beta configuration. In certain embodiments, R^{11a} is —OR^{A11} in the alpha configuration. In certain embodiments, R^{11a} is —OR^{A11} in the beta configuration. In certain embodiments, R^{A11} is hydrogen. In certain embodiments, R^{A11} is substituted or unsubstituted alkyl, e.g., substituted or unsubstituted C₁₋₆alkyl, substituted or unsubstituted C₁₋₂ alkyl, substituted or unsubstituted C₂₋₃ alkyl, substituted or unsubstituted C₃₋₄alkyl, substituted or unsubstituted C₄₋₅alkyl, or substituted or unsubstituted C₅₋₆ alkyl. In certain embodiments, R^{A11} is hydrogen, —CH₃, —CH₂CH₃, or —CH₂CH₂CH₃, i.e., to provide a group R^{11a} of formula-OH, —OCH₃, —OCH₂CH₃, or —OCH₂CH₂CH₃.

In some embodiments, each of R^{11a} and R^{11b} is hydrogen.

Groups R⁵ and R⁶—as they Relate to Formulae (F-I)-(F-Vb-ii-2)

In some embodiments R⁵ is in the alpha or beta configuration. In certain embodiments R⁵ is in the alpha configuration. In certain embodiments R⁵ is in the beta configuration. In some embodiments, the bond between C5 and C6 is a double bond and R⁵ is absent. In some embodiments, the bond between C5 and C6 is a single bond and R⁵ is hydrogen, halogen, or substituted or unsubstituted C₁₋₆alkyl. In some embodiments, the bond between C5 and C6 is a single bond and R⁵ is hydrogen, halogen, or substituted or unsubstituted C₁₋₆alkyl in the beta configuration. In some embodiments, the bond between C5 and C6 is a single bond and R⁵ is hydrogen, halogen, or substituted or unsubstituted C₁₋₆alkyl in the alpha configuration. In some embodiments, the bond between C5 and C6 is a single bond and R⁵ is hydrogen in the alpha configuration.

In certain embodiments, R⁵ is absent, hydrogen, halogen, or substituted or unsubstituted C₁₋₆alkyl. In some embodiments, R⁵ is halogen. In some embodiments, R⁵ is substituted C₁₋₆alkyl. In certain embodiments, R⁵ is substituted or unsubstituted alkyl, e.g., substituted or unsubstituted C₁₋₆alkyl, substituted or unsubstituted C₁₋₂ alkyl, substituted or unsubstituted C₂₋₃ alkyl, substituted or unsubstituted C₃₋₄ alkyl, substituted or unsubstituted C₄₋₅alkyl, or substituted or unsubstituted C₅₋₆ alkyl. In some embodiments, R⁵ is substituted or unsubstituted C₁₋₆alkyl. In some embodiments, R⁵ is unsubstituted C₁₋₆alkyl. In some embodiments, R⁵ is unsubstituted C₁ alkyl. In some embodiments, R⁵ is —CH₃.

In some embodiments, R⁶ is hydrogen, substituted or unsubstituted alkyl, or halogen. In certain embodiments, R⁶ is hydrogen. In certain embodiments, R⁶ is halogen, e.g., fluoro. In certain embodiments, R⁶ is substituted or unsubstituted alkyl, e.g., substituted or unsubstituted C₁₋₆alkyl, substituted or unsubstituted C₁₋₂ alkyl, substituted or unsubstituted C₂₋₃ alkyl, substituted or unsubstituted C₃₋₄ alkyl, substituted or unsubstituted C₄₋₅alkyl, or substituted or unsubstituted C₅₋₆ alkyl. In certain embodiments, R⁶ is C₁ alkyl, e.g., —CH₃ or CF₃. In certain embodiments, R⁶ is hydrogen, —CH₃, or —F. In certain embodiments, [symbol] represents a single bond, R⁶ is a non-hydrogen substituent in the alpha configuration. In certain embodiments, wherein [symbol] represents a single bond, R⁶ is a non-hydrogen substituent in the beta configuration.

In some embodiments, R⁶ is hydrogen.

Groups R¹⁸ and R¹⁹—as they Relate to Formulae (F-I)-(F-Vb-ii-2)

In some embodiments, R¹⁸ is hydrogen, unsubstituted C₂₋₆alkyl, substituted C₁₋₆ alkyl, or OR^{A18}, wherein each instance of R^{A18} is independently substituted or unsubstituted C₁₋₆alkyl. In some embodiments, R¹⁸ is hydrogen, unsubstituted C₂₋₆alkyl, C₁₋₆alkyl substituted with OR^{A18}, or —OR^{A18}, wherein each instance of R^{A18} is independently substituted or unsubstituted C₁₋₆alkyl. In some embodiments, R¹⁸ is hydrogen, unsubstituted C₂₋₆alkyl, C₁₋₆alkyl substituted with OR^{A18}, or —OR^{A18}, wherein each instance of R^{A18} is independently unsubstituted C₁₋₆alkyl. In some embodiments, R¹⁸ is unsubstituted C₂₋₆alkyl. In some embodiments, R¹⁸ is —CH₂CH₃, or —CH(CH₃)₂. In some embodiments, R¹⁸ is substituted C₁₋₆alkyl. In some embodiments, R¹⁸ is C₁₋₆alkyl substituted with one or more OR^{A18}, wherein each instance of R^{A18} is independently substituted or unsubstituted C₁₋₆alkyl. In some embodiments, R¹⁸ is C₁₋₆alkyl substituted with OR^{A18}, wherein R^{A18} is substituted or unsubstituted C₁₋₆alkyl. In some embodiments, R¹⁸ is —OR^{A18}, wherein R^{A18} is substituted or unsubstituted C₁₋₆alkyl. In some embodiments, R¹⁸ is hydrogen, —CH₂OCH₃, —OCH₃, —CH₂CH₃, or —CH(CH₃)₂. In some embodiments, R¹⁸ is hydrogen. In some embodiments, R¹⁸ is —CH₂CH₃.

In some embodiments, R¹⁹ is hydrogen, substituted or unsubstituted C₁₋₆alkyl, or OR^{A19}; wherein each instance of R^{A19} is independently substituted or unsubstituted C₁₋₆alkyl. In some embodiments, R¹⁹ is hydrogen, unsubstituted C₁₋₆alkyl, C₁₋₆alkyl substituted with OR^{A19}, or —OR^{A19}, wherein each instance of R^{A19} is independently substituted or unsubstituted C₁₋₆alkyl. In some embodiments, R¹⁹ is hydrogen, unsubstituted C₁₋₆alkyl, C₁₋₆alkyl substituted with OR^{A19}, or —OR^{A19}; wherein each instance of R^{A19} is independently unsubstituted C₁₋₆alkyl. In some embodiments, R¹⁹ is unsubstituted C₁₋₆alkyl. In some embodiments, R¹⁹ is —CH₃, —CH₂CH₃, or —CH(CH₃)₂. In some embodiments, R¹⁹ is —CH₃. In some embodiments, R¹⁹ is —CH₂CH₃, or —CH(CH₃)₂. In some embodiments, R¹⁹ is substituted C₁₋₆alkyl. In some embodiments, R¹⁹ is C₁₋₆alkyl substituted with one or more OR^{A19}; wherein each instance of R^{A19} is independently substituted or unsubstituted C₁₋₆alkyl. In some embodiments, R¹⁹ is C₁₋₆alkyl substituted with OR^{A19}, wherein R^{A19} is substituted or unsubstituted C₁₋₆alkyl. In some embodiments, R¹⁹ is —OR^{A19}; wherein R^{A19} is substituted or unsubstituted C₁₋₆alkyl. In some embodiments, R¹⁹ is hydrogen, —CH₃, —CH₂OCH₃, —OCH₃, —CH₂CH₃, or —CH(CH₃)₂. In some embodiments, R¹⁹ is hydrogen, —CH₂OCH₃, —OCH₃, —CH₂CH₃, or —CH(CH₃)₂. In some embodiments, R¹⁹ is hydrogen, —CH₃, or —CH₂CH₃. In some embodiments, R¹⁹ is —CH₃. In some embodiments, R¹⁹ is —CH₂CH₃. In some embodiments, R¹⁹ is hydrogen or —CH₃. In some embodiments, R¹⁹ is hydrogen. In some embodiments, R¹⁹ is —CH₃.

Groups X, n, R^{23a}, R^{23b}, R^{24a}, and R^{24b}—as they Relate to Formulae (F-I)-(F-Vb-ii-2)

In certain embodiments, X is —(C(R^{X})₂)ₙ— or —O—, wherein R^{X} is hydrogen or fluorine, or one R^{X} group and R^{23b} are joined to form a double bond.

In certain embodiments, X is —O—. In certain embodiments, X is —CH₂—. In certain embodiments, X is —CF₂—.

In certain embodiments, n is selected from 1, 2 or 3. In some embodiments, n is 1. In some embodiments, n is 2. In some embodiments, n is 3.

In some embodiments, X is —(C(R^{X})₂—, wherein R^{X} is hydrogen or fluorine. In some embodiments, X is —CH₂—. In some embodiments, X is —CF₂—.

In some embodiments, X is —(C(R^{X})₂)₂—, wherein R^{X} is hydrogen or fluorine. In some embodiments, X is —(CH₂)₂—. In some embodiments, X is —(CF₂)₂—.

In some embodiments, each of R^{23a} and R^{23b} is independently hydrogen or fluorine. In some embodiments, each of R^{23a} and R^{23b} is hydrogen. In some embodiments, at least one of R^{23a} and R^{23b} is fluorine. In some embodiments, each of R^{23a} and R^{23b} is fluorine. In certain embodiments, at least one of R^{23a} and R^{23b} is hydrogen. In certain embodiments, R^{X} and R^{23b} are joined to form a double bond, e.g., cis or trans double bond.

In some embodiments, R^{24a} is selected from the group consisting of substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl; and R^{24b} is hydrogen or substituted or unsubstituted C₁₋₆alkyl; or R^{24a} and R^{24b}, taken together with the carbon atom to which they are attached, form a substituted or unsubstituted C₃₋₆carbocyclyl, or a substituted or unsubstituted 3- to 10-membered heterocyclyl.

In some embodiments, R^{24a} is selected from the group consisting of substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl; and R^{24b} is hydrogen or a substituted or unsubstituted C₁₋₆alkyl; or R^{24a} and R^{24b}, taken together with the carbon atom to which they are attached, form a substituted or unsubstituted C₃₋₆carbocyclyl, or a substituted or unsubstituted 3- to 10-membered heterocyclyl; provided that when R^{24b} is H, R^{24a} is not C₁₋₆alkyl substituted with CN. In some embodiments, R^{24a} is a non-hydrogen group selected from substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C6-14aryl, or substituted or unsubstituted 5- to 10-membered heteroaryl. In some embodiments, R^{24a} is a substituted or unsubstituted C₂₋₄ alkyl, substituted or unsubstituted C₂₋₃alkenyl, substituted or unsubstituted C₂₋₃ alkynyl, or substituted or unsubstituted C₃ carbocyclyl. In some embodiments, R^{24a} is a substituted or unsubstituted C₁-4 alkyl, substituted or unsubstituted C₂₋₃alkenyl, substituted or unsubstituted C₂₋₃ alkynyl, or substituted or unsubstituted C₃ carbocyclyl. In some embodiments, R^{24a} is a non-hydrogen group substituted with fluorine. In some embodiments, R^{24a} is a non-hydrogen group substituted with one or more —OR^{A24} groups, wherein R^{A24} is hydrogen or substituted or unsubstituted C₁₋₆alkyl.

In some embodiments, R^{24a} is a substituted or unsubstituted C₁₋₆alkyl. In some embodiments, R^{24a} is unsubstituted C₁₋₆alkyl. In some embodiments, R^{24a} is substituted C₁₋₆alkyl. In some embodiments, R^{24a} is —CH₃, —CH₂CH₃, —CH(CH₃)₂, or —CF₃. In some embodiments R^{24a} is —CH₃, —CH(CH₃)₂ or —CF₃. In some embodiments, R^{24a} is —CH₃. In some embodiments, R^{24a} is —CH(CH₃)₂. In some embodiments, R^{24a} is —CF₃. In some embodiments, R^{24a} is —CH₂OR^{A24}, —CH₂CH₂OR^{A24}, or —CH₂CH₂CH₂OR^{A24}; wherein R^{A24} is hydrogen or substituted or unsubstituted C₁₋₆alkyl.

In some embodiments, R^{24b} is hydrogen, substituted or unsubstituted C₁₋₆alkyl group, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, or substituted or unsubstituted 5- to 10-membered heteroaryl.

In some embodiments, R^{24b} is hydrogen or substituted or unsubstituted C₁₋₆alkyl. In some embodiments, R^{24b} is hydrogen or unsubstituted C₁₋₆alkyl. In some embodiments, R^{24b} is hydrogen. In some embodiments R^{24b} is unsubstituted C₁₋₆alkyl. In some embodiments, R^{24b} is —CH₃ or —CF₃. In some embodiments, R^{24b} is —CH₃.

In some embodiments, R^{24a} is —CF₃ and R^{24b} is hydrogen or C₁₋₆alkyl. In some embodiments, R^{24a} is a non-hydrogen group substituted with fluorine, and R^{24b} is —CH₃.

In some embodiments, R^{24a} is a substituted or unsubstituted C₂₋₄ alkyl, substituted or unsubstituted C₂₋₃alkenyl, substituted or unsubstituted C₂₋₃ alkynyl, or substituted or unsubstituted C₃ carbocyclyl; and R^{24b} is —CH₃. In some embodiments, R^{24a} is a substituted or unsubstituted C₁₋₄ alkyl, substituted or unsubstituted C₂₋₃alkenyl, substituted or unsubstituted C₂₋₃ alkynyl, or substituted or unsubstituted C₃ carbocyclyl, and R^{24b} is —H. In some embodiments, R^{24a} is a unsubstituted C₂₋₄ alkyl, unsubstituted C₂₋₃alkenyl, or unsubstituted C₂₋₃ alkynyl, or unsubstituted C₃ carbocyclyl, and R^{24b} is —H. In some embodiments, R^{24a} is a unsubstituted C₂₋₄ alkyl, unsubstituted C₂₋₃alkenyl, or unsubstituted C₂₋₃ alkynyl, or unsubstituted C₃ carbocyclyl; and R^{24b} is —CH₃. In some embodiments, when R^{24b} is H, R^{24a} is not C₁₋₆alkyl substituted with CN.

In some embodiments, R^{24a} is a non-hydrogen group substituted with fluorine, and R^{24b} is —CH₃.

In some embodiments, R^{24a} is —CH(CH₃)₂ or —CF₃, and R^{24b} is hydrogen.

In some embodiments, R^{24a} and R^{24b}, taken together with the carbon atom to which they are attached, form a substituted or unsubstituted C₃₋₆carbocyclyl, or a substituted or unsubstituted 3- to 10-membered heterocyclyl. In some embodiments, R^{24a} and R^{24b}, taken together with the carbon atom to which they are attached, form a substituted or unsubstituted C₃₋₆carbocyclyl. In some embodiments, R^{24a} and R^{24b}, taken together with the carbon atom to which they are attached, form an unsubstituted C₃₋₆carbocyclyl. In some embodiments, R^{24a} and R^{24b} taken together with the carbon atom to which they are attached, form a cyclopropyl. In some embodiments, R^{24a} and R^{24b} are taken together with the carbon atoms to which they are attached, to form an unsubstituted 3- to 10-membered heterocyclyl. In some embodiments, R^{24a} and R^{24b} are taken together with the carbon atoms to which they are attached, to form an unsubstituted 3- to 6-membered heterocyclyl.

In some embodiments, R³ is C₁₋₃ alkyl, R^{24a} is a non-hydrogen group substituted with fluorine, and R^{24b} is —CH₃.

In some embodiments, R³ is C₁₋₃ alkyl, R^{24a} is a non-hydrogen group substituted with fluorine, and R^{24b} is hydrogen.

In some embodiments, R³ is C₁₋₃ alkyl, R^{24a} is selected from the group consisting of substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, and R^{24b} is —CH₃. In some embodiments, R³ is C₁₋₃ alkyl; R^{24a} is selected from the group consisting of substituted or unsubstituted C₁₋₆alkyl, unsubstituted C₂₋₆alkenyl, unsubstituted C₂₋₆alkynyl, or unsubstituted C₃₋₆carbocyclyl; and R^{24b} is —CH₃. In some embodiments, R³ is —CH₃ or CH₂CH₃; R^{24a} is selected from the group consisting of substituted or unsubstituted C₁₋₆alkyl, unsubstituted C₂₋₆alkenyl, unsubstituted C₂₋₆alkynyl, or unsubstituted C₃₋₆carbocyclyl; and and R^{24b} is —CH₃. In some embodiments, R³ is C₁₋₃ alkyl; R^{24a} is selected from a substituted or unsubstituted C₁₋₆alkyl; and R^{24b} is —CH₃. In some embodiments, R³ is CH₃ or —CH₂CH₃; R^{24a} is selected from a substituted or unsubstituted C₁₋₆alkyl; and R^{24b} is —CH₃.

In some embodiments, R³ is —CH₃ or —CH₂CH₃ and at least one of R^{23a} and R^{23b} is fluorine or R^{23a} and R^{23b} are both hydrogen.

In some embodiments, R³ is —CH₃ or —CH₂CH₃ and R^{24a} is a non-hydrogen group substituted with fluorine or one or more —OR^{A24} groups, wherein R^{A24} is hydrogen or substituted or unsubstituted C₁₋₆alkyl.

In some embodiments, R³ is —CH₃ or —CH₂CH₃ and R^{24b} is —CH₃ or —CF₃. In some embodiments, R³ is —CH₃ or —CH₂CH₃; R^{24b} is —CH₃ or —CF₃; and R^{24a} is selected from the group consisting of substituted or unsubstituted C₁₋₆alkyl, unsubstituted C₂₋₆alkenyl, unsubstituted C₂₋₆alkynyl, or unsubstituted C₃₋₆carbocyclyl.

In some embodiments, R³ is —CH₃ or —CH₂CH₃, R^{24a} is —CH(CH₃)₂ or —CF₃; and R^{24b} is hydrogen.

q, r, s—as they Relate to Formulae (F-I)-(F-Vb-ii-2)

In some embodiments, q is 0, 1 or 2. In some embodiments, q is 0. In some embodiments, q is 1. In some embodiments, q is 2.

In some embodiments, u is 1 or 2. In some embodiments, u is 1. In some embodiments, r is 2.

In some embodiments, s is 1 or 2. In some embodiments, s is 1. In some embodiments, s is 2.

In some embodiments, q is 0 and r is 1. In some embodiments, q is 1 and r is 1. In some embodiments, q is 0, r is 1, and s is 1. In some embodiments, q is 0, r is 1, s is 1 and t is 0. In some embodiments, q, r, and s are each independently 1.

In some embodiments, the compound of Formula (F-I) is any one of Compounds F-1 to F-14 in Table F-1 and pharmaceutically acceptable salts thereof. In some embodiments, the compound of Formula (F-I) is any one of Compounds F-1 to F-14 in Table F-1. In some embodiments, the compound of Formula (F-I) is a pharmaceutically acceptable salt of any one of Compounds F-1 to F-14 in Table F-1.

TABLE F-1

| Cmpd | Structure |
|---|---|
| F-1 | 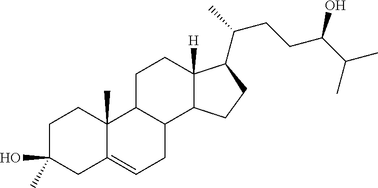 |
| F-2 | 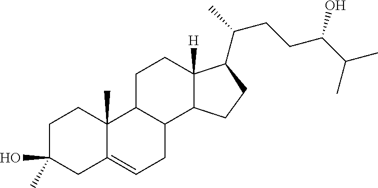 |
| F-3 | 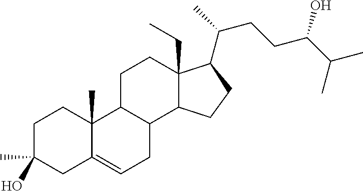 |
| F-4 | 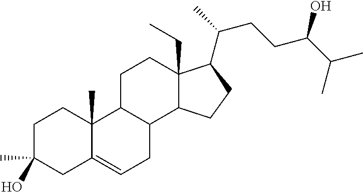 |
| F-5 | 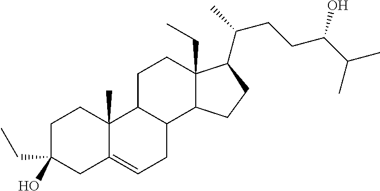 |
| F-6 | 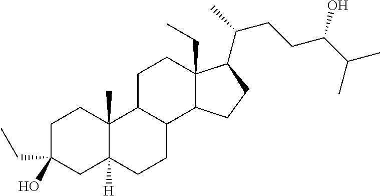 |
| F-7 | 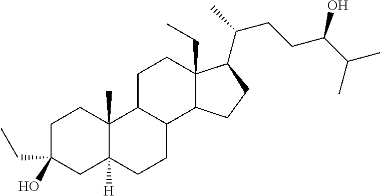 |
| F-8 | 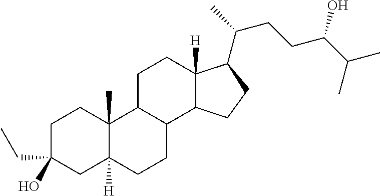 |
| F-9 | 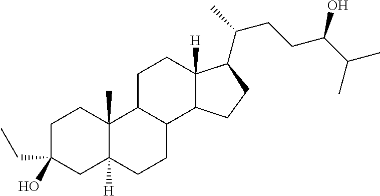 |
| F-10 | 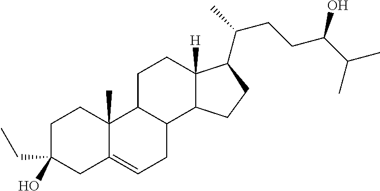 |
| F-11 | 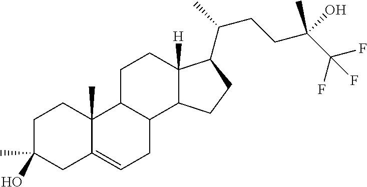 |
| F-12 | 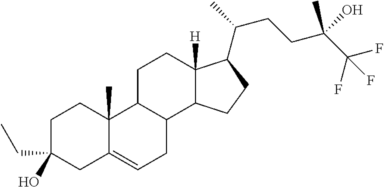 |
| F-13 | 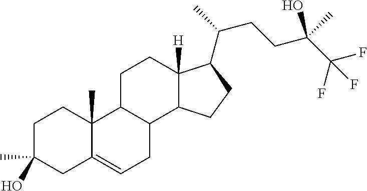 |
| F-14 | 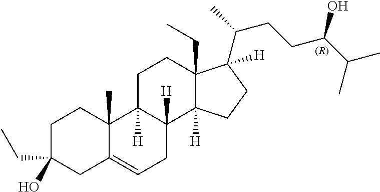 |

In one aspect, the present disclosure provides a compound of Formula (G-I):

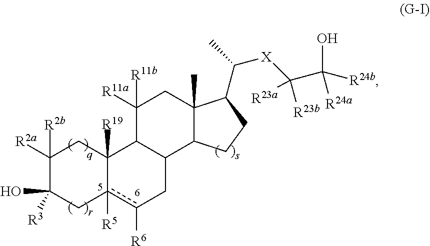

or a pharmaceutically acceptable salt thereof;
wherein:
   each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein R^{A2} is hydrogen or substituted or unsubstituted C₁₋₆alkyl;
   R³ is substituted or unsubstituted C₁₋₆alkyl, substituted or unsubstituted C₂₋₆ alkenyl, or substituted or unsubstituted C₂₋₆alkynyl;
   R⁵ is absent, hydrogen, substituted or unsubstituted C₁₋₆alkyl, or halogen;
   R⁶ is hydrogen, substituted or unsubstituted C₁₋₆alkyl, or halogen;
   R¹⁹ is hydrogen, substituted or unsubstituted C₂₋₆alkyl, or OR^{A19}; wherein each instance of R^{A19} is independently substituted or unsubstituted C₁₋₆alkyl;
   R^{11a} is hydrogen or —OR^{A11}, wherein R^{A11} is hydrogen or substituted or unsubstituted C₁₋₆alkyl, and R^{11b} is hydrogen; or R^{11a} and R^{11b} are joined to form an oxo (═O) group;
   X is —(C(R^{X})₂)ₙ— or —O—, wherein R^{X} is hydrogen or fluorine, or one R^{X} group and R^{23b} are joined to form a double bond;
   n is 1, 2 or 3;
   each of R^{23a} and R^{23b} is independently hydrogen or fluorine;
   R^{24a} is selected from the group consisting of substituted or unsubstituted C₁₋₆ alkyl, substituted or unsubstituted C₂₋₆alkenyl, substituted or unsubstituted C₂₋₆ alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl; and
   R^{24b} is hydrogen or a substituted or unsubstituted C₁₋₆alkyl;
   or R^{24a} and R^{24b}, taken together with the carbon atom to which they are attached, form a substituted or unsubstituted C₃₋₆carbocyclyl;
   q is 0, 1 or 2;
   r is 1 or 2;
s is 1, 2, 3 or 4; and
   [symbol] represents a single or double bond; provided if the bond between C5 and C6 is a double bond, R⁵ is absent.

In some embodiments, the compound of Formula (G-I) is a compound of Formula (G-Ia):

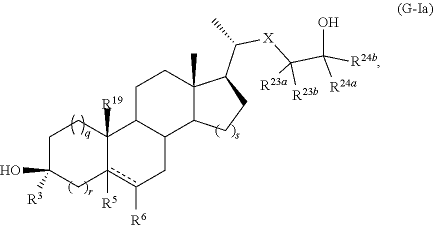

or a pharmaceutically acceptable salt thereof.

In some embodiments, the compound of Formula (G-Ia) is a compound of Formula (G-Ia-1):

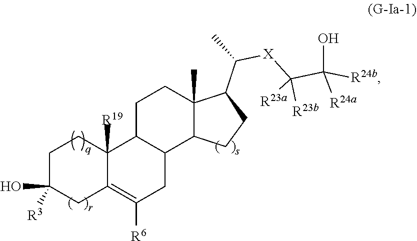

or a pharmaceutically acceptable salt thereof.

In some embodiments, the compound of Formula (G-Ia) is a compound of Formula (G-Ia-2):

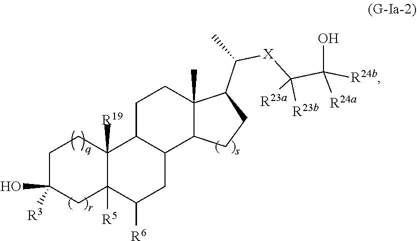

or a pharmaceutically acceptable salt thereof.

In some embodiments, the compound of Formula (G-I) is a compound of Formula (G-II):

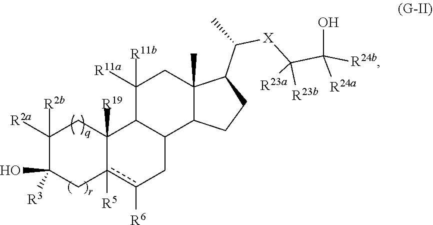

or a pharmaceutically acceptable salt thereof.

In some embodiments, the compound of Formula (G-II) is a compound of Formula (IIa):

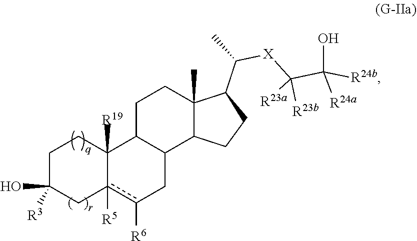

or a pharmaceutically acceptable salt thereof.

In some embodiments, the compound of Formula (G-IIa) is a compound of Formula (G-IIa-1):

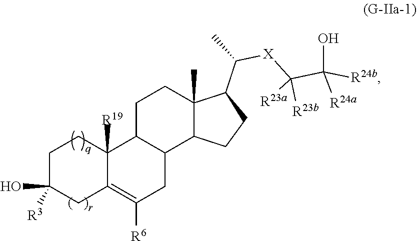

or a pharmaceutically acceptable salt thereof.

In some embodiments, the compound of Formula (G-IIa) is a compound of Formula (G-IIa-2):

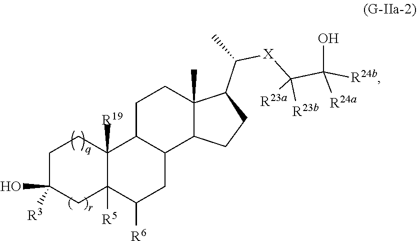

or a pharmaceutically acceptable salt thereof.

In some embodiments, the compound of Formula (G-I) is a compound of Formula (G-III):

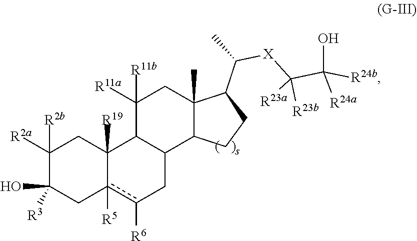

or a pharmaceutically acceptable salt thereof.

In some embodiments, the compound of Formula (G-III) is a compound of Formula (G-IIIa):

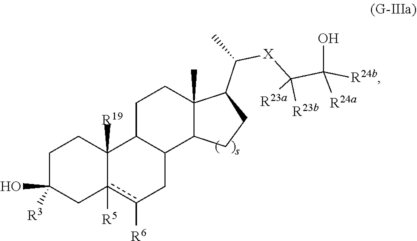

or a pharmaceutically acceptable salt thereof.

In some embodiments, the compound of Formula (G-IIIa) is a compound of Formula (G-IIIa-1):

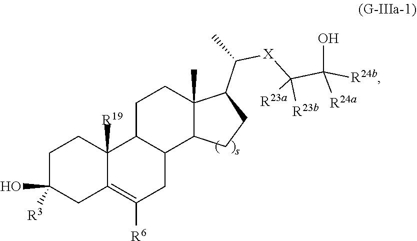

or a pharmaceutically acceptable salt thereof.

In some embodiments, the compound of Formula (G-IIIa) is a compound of Formula (G-IIIa-2):

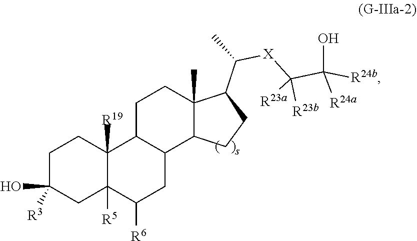

or a pharmaceutically acceptable salt thereof.

In some embodiments, the compound of Formula (G-I) is a compound of Formula (G-IV):

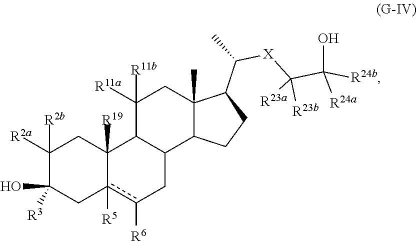

or a pharmaceutically acceptable salt thereof.

In some embodiments, the compound of Formula (G-IV) is a compound of Formula (G-IVa):

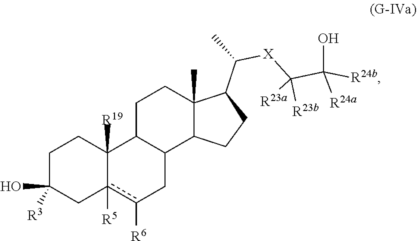

or a pharmaceutically acceptable salt thereof.

In some embodiments, the compound of Formula (G-IVa) is a compound of Formula (G-IVa-1):

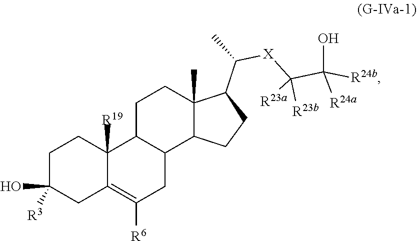

or a pharmaceutically acceptable salt thereof.

In some embodiments, the compound of Formula (G-IVa) is a compound of Formula (G-IVa-2):

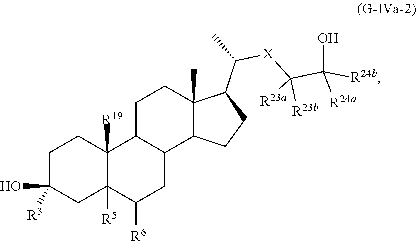

or a pharmaceutically acceptable salt thereof.

Groups R^{2a} and R^{2b}—as they Relate to Formulae (G-I)-(G-IVa-2)

In certain embodiments, R^{2a} and R^{2b} is each independently hydrogen, halogen, substituted or unsubstituted C₁₋₆alkyl, substituted or unsubstituted C₃₋₁₀carbocyclyl, or OR^{A2}, wherein R^{A2} is hydrogen or substituted or unsubstituted C₁₋₆alkyl. In certain embodiments, each R^{2a} and R^{2b} is hydrogen. In certain embodiments, each of R^{2a} and R^{2b} is independently halogen, e.g., fluoro, chloro, bromo, or iodo. In certain embodiments, each of R^{2a} and R^{2b} is independently fluoro or chloro. In certain embodiments, each of R^{2a} and R^{2b} is independently is substituted or unsubstituted C₁₋₆alkyl, e.g., substituted or unsubstituted C₁₋₂ alkyl, substituted or unsubstituted C₂₋₃alkyl, substituted or unsubstituted C₃₋₄ alkyl, substituted or unsubstituted C₄₋₅alkyl, or substituted or unsubstituted C₅₋₆alkyl. In certain embodiments, each of R^{2a} and R^{2b} is independently —CH₃, —CH₂CH₃, —CH₂CH₂CH₃, or cyclopropyl. In certain embodiments, each of R^{2a} and R^{2b} is independently —OR^{A2}. In certain embodiments, R^{A2} is hydrogen. In certain embodiments, R^{A2} is substituted or unsubstituted alkyl, e.g., substituted or unsubstituted C₁₋₆alkyl, substituted or unsubstituted C₁₋₂alkyl, substituted or unsubstituted C₂₋₃alkyl, substituted or unsubstituted C₃₋₄alkyl, substituted or unsubstituted C₄₋₅alkyl, or substituted or unsubstituted C₅₋₆alkyl. In certain embodiments, R^{A2} is hydrogen, —CH₃, —CH₂CH₃, or —CH₂CH₂CH₃, i.e., to provide a group of R^{2a} or R^{2b} each independently of formula-OH, —OCH₃, —OCH₂CH₃, or —OCH₂CH₂CH₃. In certain embodiments, each of R^{2a} and R^{2b} is independently a non-hydrogen substituent. In certain embodiments, one of R^{2a} and R^{2b} is a non-hydrogen substituent in the alpha configuration. In certain embodiments, one of R^{2a} and R^{2b} is a non-hydrogen substituent in the beta configuration.

### Group R³—as it Relates to Formulae (G-I)-(G-IVa-2)

In certain embodiments, R³ is substituted or unsubstituted aliphatic, i.e., substituted or unsubstituted alkyl, substituted or unsubstituted alkenyl, substituted or unsubstituted alkynyl, or substituted or unsubstituted carbocyclyl.

In certain embodiments, R³ is substituted or unsubstituted C₁₋₆alkyl, substituted or unsubstituted C₂₋₆alkenyl, substituted or unsubstituted C₂₋₆alkynyl, or substituted or unsubstituted C₃₋₁₀carbocyclyl.

In certain embodiments, R³ is substituted or unsubstituted alkyl, e.g., substituted or unsubstituted C₁₋₆alkyl, substituted or unsubstituted C₁₋₂ alkyl, substituted or unsubstituted C₂₋₃ alkyl, substituted or unsubstituted C₃₋₄ alkyl, substituted or unsubstituted C₄₋₅ alkyl, or substituted or unsubstituted C₅₋₆ alkyl. Exemplary R³ C₁₋₆alkyl groups include, but are not limited to, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), n-hexyl (C₆), C₁₋₆ alkyl substituted with 1, 2, 3, 4, 5, 6, 7, 8, 9, 10, or more fluoro groups (e.g., —CF₃, —CH₂F, —CHF₂, difluoroethyl, and 2,2,2-trifluoro-1,1-dimethyl-ethyl), C₁₋₆alkyl substituted with 1, 2, 3, 4, 5, 6, 7, 8, 9, 10, or more chloro groups (e.g., —CH₂Cl, —CHCl₂), and C₁₋₆alkyl substituted with 1, 2, 3, 4, 5, 6, 7, 8, 9, 10, or more alkoxy groups (e.g., —CH₂OCH₃, —CH₂OCH₂CH₃, —CH₂O-cyclopropyl). In certain embodiments, R³ is substituted alkyl, e.g., R³ is haloalkyl, alkoxyalkyl, or aminoalkyl. In certain embodiments, R³ is Me, Et, n-Pr, n-Bu, i-Bu, fluoromethyl, chloromethyl, difluoromethyl, trifluoromethyl, trifluoroethyl, difluoroethyl, 2,2,2-trifluoro-1,1-dimethyl-ethyl, methoxymethyl, methoxyethyl, or ethoxymethyl.

In certain embodiments, R³ is substituted or unsubstituted C₁₋₆alkyl. In certain embodiments, R³ is unsubstituted C₁₋₆alkyl. In some embodiments, R³ is selected from the group consisting of CH₃, —CH₂CH₃, and —CH₂CH₂CH₃. In some embodiments, R³ is ethyl.

In certain embodiments, R³ is substituted C₁₋₆alkyl. In certain embodiments, R³ is alkyl substituted with one or more fluorine atoms, e.g., R³ is —CH₂F, —CHF₂, or —CF₃. In certain embodiments, R³ is alkyl substituted with one or more —OR^{A3} groups, wherein R^{A3} is hydrogen or substituted or unsubstituted alkyl. In certain embodiments, R³ is —CH₂OR^{A3}, e.g., wherein R^{A3} is hydrogen, —CH₃, —CH₂CH₃, or —CH₂CH₂CH₃.

In certain embodiments, R³ is —CH₃, —CH₂CH₃, —CH₂CH₂CH₃, or —CH₂OCH₃. In certain embodiments, R³ is —CH₂CH₃. In certain embodiments, R³ is —CH₂OCH₃.

In certain embodiments, R³ is substituted or unsubstituted alkenyl, e.g., substituted or unsubstituted C₂₋₆alkenyl, substituted or unsubstituted C₂₋₃alkenyl, substituted or unsubstituted C₃₋₄alkenyl, substituted or unsubstituted C₄₋₅alkenyl, or substituted or unsubstituted C₅₋₆alkenyl. In certain embodiments, R³ is ethenyl (C₂), propenyl (C₃), or butenyl (C₄), unsubstituted or substituted with one or more substituents selected from the group consisting of alkyl, halo, haloalkyl, alkoxyalkyl, or hydroxyl. In certain embodiments, R³ is ethenyl, propenyl, or butenyl, unsubstituted or substituted with alkyl, halo, haloalkyl, alkoxyalkyl, or hydroxy. In certain embodiments, R³ is ethenyl.

In certain embodiments, R³ is substituted or unsubstituted alkynyl, e.g., substituted or unsubstituted C₂₋₆alkynyl, substituted or unsubstituted C₂₋₃alkynyl, substituted or unsubstituted C₃₋₄alkynyl, substituted or unsubstituted C₄₋₅alkynyl, or substituted or unsubstituted C₅₋₆alkynyl. Exemplary substituted or unsubstituted R³ alkynyl groups include, but are not limited to, ethynyl, propynyl, or butynyl, unsubstituted or substituted with alkyl, halo, haloalkyl (e.g., CF₃), alkoxyalkyl, cycloalkyl (e.g., cyclopropyl or cyclobutyl), or hydroxyl. In certain embodiments, R³ is selected from the group consisting of trifluoroethynyl, cyclopropylethynyl, cyclobutylethynyl, and propynyl, fluoropropynyl, and chloroethynyl. In certain embodiments, R³ is ethynyl (C₂), propynyl (C₃), or butynyl (C₄), unsubstituted or substituted with one or more substituents selected from the group consisting of substituted or unsubstituted aryl, substituted or unsubstituted heteroaryl, substituted or unsubstituted carbocyclyl, and substituted or unsubstituted heterocyclyl. In certain embodiments, R³ is ethynyl (C₂), propynyl (C₃), or butynyl (C₄) substituted with substituted phenyl. In certain embodiments, the phenyl substituent is further substituted with one or more substituents selected from the group consisting of halo, alkyl, trifluoroalkyl, alkoxy, acyl, amino or amido. In certain embodiments, R³ is ethynyl (C₂), propynyl (C₃), or butynyl (C₄) substituted with substituted or unsubstituted pyrrolyl, imidazolyl, pyrazolyl, oxazoyl, thiazolyl, isoxazoyl, 1,2,3-triazolyl, 1,2,4-triazolyl, oxadiazolyl, thiadiazolyl, or tetrazolyl.

In certain embodiments, R³ is ethynyl, propynyl, or butynyl, unsubstituted or substituted with alkyl, halo, haloalkyl, alkoxyalkyl, or hydroxyl. In certain embodiments, R³ is ethynyl or propynyl, substituted with substituted or unsubstituted aryl. In certain embodiments, R³ is ethynyl or propynyl, substituted with phenyl unsubstituted or substituted with halo, alkyl, alkoxy, haloalkyl, trihaloalkyl, or acyl. In certain embodiments, R³ is ethynyl or propynyl, substituted with substituted or unsubstituted carbocyclyl. In certain embodiments, R³ is ethynyl or propynyl, substituted with substituted or unsubstituted cyclopropyl, cyclobutyl, cyclopentyl, or cyclohexyl. In certain embodiments, R³ is ethynyl or propynyl, substituted with substituted or unsubstituted heteroaryl. In certain embodiments, R³ is ethynyl or propynyl, substituted with substituted or unsubstituted pyridinyl, or pyrimidinyl. In certain embodiments, R³ is ethynyl or propynyl, substituted with substituted or unsubstituted pyrrolyl, imidazolyl, pyrazolyl, oxazoyl, thiazolyl, isoxazoyl, 1,2,3-triazolyl, 1,2,4-triazolyl, oxadiazolyl, thiadiazolyl, tetrazolyl. In certain embodiments, R³ is ethynyl or propynyl, substituted with substituted or unsubstituted heterocyclyl. In certain embodiments, R³ is ethynyl or propynyl, substituted with substituted or unsubstituted pyrrolidinyl, piperidinyl, piperazinyl, or mopholinyl. In certain embodiments, R³ is propynyl or butynyl, substituted with hydroxyl or alkoxy. In certain embodiments, R³ is propynyl or butynyl, substituted with methoxy or ethoxy. In certain embodiments, R³ is ethynyl or propynyl, substituted with chloro. In certain embodiments, R³ is ethynyl or propynyl, substituted with trifluoromethyl.

In certain embodiments, R³ is substituted or unsubstituted carbocyclyl, e.g., substituted or unsubstituted C₃₋₁₀carbocyclyl, substituted or unsubstituted C₃₋₆carbocyclyl, substituted or unsubstituted C₃₋₄ carbocyclyl, substituted or unsubstituted C₄₋₅ carbocyclyl, or substituted or unsubstituted C₅₋₆ carbocyclyl. In certain embodiments, R³ is substituted or unsubstituted cyclopropyl or substituted or unsubstituted cyclobutyl.

In certain embodiments, R³ is in the alpha configuration. In certain embodiments, R³ is in the beta configuration.

Groups R⁵ and R⁶—as they Relate to Formulae (G-I)-(G-IVa-2)

In some embodiments, R⁵ is in the alpha or beta configuration. In certain embodiments, R⁵ is in the alpha configuration. In certain embodiments, R⁵ is in the beta configuration. In some embodiments, when the bond between C5 and C6 is a double bond, R⁵ is absent. In some embodiments, the bond between C5 and C6 is a single bond and R⁵ is in the alpha configuration. In some embodiments, the bond between C5 and C6 is a single bond and R⁵ is hydrogen in the alpha configuration.

In certain embodiments, R⁵ is absent, hydrogen, halogen, or substituted or unsubstituted C₁₋₆alkyl. In some embodiments, R⁵ is halogen. In some embodiments, R⁵ is substituted C₁₋₆alkyl. In certain embodiments, R⁵ is substituted or unsubstituted alkyl, e.g., substituted or unsubstituted C₁₋₆alkyl, substituted or unsubstituted C₁₋₂ alkyl, substituted or unsubstituted C₂₋₃ alkyl, substituted or unsubstituted C₃₋₄ alkyl, substituted or unsubstituted C₄₋₅alkyl, or substituted or unsubstituted C₅₋₆ alkyl. In some embodiments, R⁵ is substituted or unsubstituted C₁₋₆alkyl. In some embodiments, R⁵ is unsubstituted C₁₋₆alkyl. In some embodiments, R⁵ is unsubstituted C₁ alkyl. In some embodiments, R⁵ is —CH₃. In some embodiments, R⁵ is hydrogen.

In certain embodiments, R⁶ is hydrogen, halogen, or substituted or unsubstituted C₁₋₆alkyl. In certain embodiments, R⁶ is halogen, e.g., fluoro. In certain embodiments, R⁶ is substituted or unsubstituted alkyl, e.g., substituted or unsubstituted C₁₋₆alkyl, substituted or unsubstituted C₁₋₂ alkyl, substituted or unsubstituted C₂₋₃ alkyl, substituted or unsubstituted C₃₋₄alkyl, substituted or unsubstituted C₄₋₅alkyl, or substituted or unsubstituted C₅₋₆alkyl. In certain embodiments, R⁶ is C₁ alkyl, e.g., —CH₃ or —CF₃. In certain embodiments, R⁶ is hydrogen, —CH₃, or —F. In certain embodiments, [symbol] represents a single bond and R⁶ is a non-hydrogen substituent in the alpha configuration. In certain embodiments, [symbol] represents a single bond and R⁶ is a non-hydrogen substituent in the beta configuration.

Groups R^{11a} and R^{11b}—as they Relate to Formulae (G-I)-(G-IVa-2)

In certain embodiments, R^{11a} is hydrogen or —OR^{A11}, wherein R^{A11} is hydrogen or substituted or unsubstituted C₁₋₆alkyl, and R^{11b} is hydrogen; or R^{11a} and R^{11b} are joined to form an oxo (═O) group.

In certain embodiments, R^{11a} and R^{11b} are joined to form an oxo (═O) group.

In certain embodiments, R^{11a} is —OR^{A11} and R^{11b} is hydrogen. In certain embodiments, R^{11a} is —OR^{A11} in the alpha or beta configuration. In certain embodiments, R^{11a} is —OR^{A11} in the alpha configuration. In certain embodiments, R^{11a} is —OR^{A11} in the beta configuration. In certain embodiments, R^{A11} is hydrogen. In certain embodiments, R^{A11} is substituted or unsubstituted alkyl, e.g., substituted or unsubstituted C₁₋₆alkyl, substituted or unsubstituted C₁₋₂ alkyl, substituted or unsubstituted C₂₋₃ alkyl, substituted or unsubstituted C₃₋₄alkyl, substituted or unsubstituted C₄₋₅alkyl, or substituted or unsubstituted C₅₋₆ alkyl. In certain embodiments, R^{A11} is hydrogen, —CH₃, —CH₂CH₃, or —CH₂CH₂CH₃, i.e., to provide a group R^{11a} of the formula-OH, —OCH₃, —OCH₂CH₃, or —OCH₂CH₂CH₃.

In some embodiments, each of R^{11a} and R^{11b} is hydrogen.

### Group R¹⁹—as it Relates to Formulae (G-I)-(G-IVa-2)

In some embodiments, R¹⁹ is hydrogen, substituted or unsubstituted C₂₋₆alkyl, or OR^{A19}; wherein each instance of R^{A19} is independently substituted or unsubstituted C₁₋₆alkyl.

In some embodiments, R¹⁹ is hydrogen, unsubstituted C₂₋₆alkyl, C₂₋₆alkyl substituted with OR^{A19}, or —OR^{A19}; wherein each instance of R^{A19} is independently substituted or unsubstituted C₁₋₆alkyl. In some embodiments, R¹⁹ is hydrogen, unsubstituted C₂₋₆alkyl, C₂₋₆alkyl substituted with OR^{A19}, or —OR^{A19}; wherein each instance of R^{A19} is independently unsubstituted C₁₋₆alkyl. In some embodiments, R¹⁹ is unsubstituted C₂₋₆alkyl. In some embodiments, R¹⁹ is —CH₂CH₃ or —CH(CH₃)₂. In some embodiments, R¹⁹ is substituted C₂₋₆ alkyl. In some embodiments, R¹⁹ is C₂₋₆alkyl substituted with one or more OR^{A19}; wherein each instance of R^{A19} is independently substituted or unsubstituted C₁₋₆alkyl. In some embodiments, R¹⁹ is C₂₋₆alkyl substituted with OR^{A19}, wherein R^{A19} is substituted or unsubstituted C₁₋₆alkyl. In some embodiments, R¹⁹ is —OR^{A19}; wherein R^{A19} is substituted or unsubstituted C₁₋₆alkyl. In some embodiments, R¹⁹ is hydrogen, —OCH₃, —CH₂CH₃, or CH(CH₃)₂. In some embodiments, R¹⁹ is hydrogen. In some embodiments, R¹⁹ is —CH₂CH₃.

Groups X, n, R^{23a}, R^{23b}, R^{24a}, and R^{24b}—as they Relate to Formulae (G-I)-(G-IVa-2)

In certain embodiments, X is —(C(R^{X})₂)ₙ— or —O—, wherein R^{X} is hydrogen or fluorine, or one R^{X} group and R^{23b} are joined to form a double bond.

In certain embodiments, X is —O—. In certain embodiments, X is —CH₂—. In certain embodiments, X is —CF₂—.

In certain embodiments, n is selected from 1, 2 or 3. In some embodiments, n is 1. In some embodiments, n is 2. In some embodiments, n is 3.

In some embodiments, X is —(C(R^{X})₂—, wherein R^{X} is hydrogen or fluorine. In some embodiments, X is —CH₂—. In some embodiments, X is —CF₂—.

In some embodiments, X is —(C(R^{X})₂)₂—, wherein R^{X} is hydrogen or fluorine. In some embodiments, X is —(CH₂)₂—. In some embodiments, X is —(CF₂)₂—.

In some embodiments, each of R^{23a} and R^{23b} is independently hydrogen or fluorine. In some embodiments, each of R^{23a} and R^{23b} is hydrogen. In some embodiments, at least one of R^{23a} and R^{23b} is fluorine. In some embodiments, each of R^{23a} and R^{23b} is fluorine. In certain embodiments, at least one of R^{23a} and R^{23b} is hydrogen. In certain embodiments, R^{X} and R^{23b} are joined to form a double bond, e.g., cis or trans double bond.

In some embodiments, R^{24a} is selected from the group consisting of substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl; and R^{24b} is hydrogen or a substituted or unsubstituted C₁₋₆alkyl; or R^{24a} and R^{24b}, taken together with the carbon atom to which they are attached, form a substituted or unsubstituted C₃₋₆carbocyclyl or a substituted or unsubstituted 3- to 10-membered heterocyclyl.

In some embodiments, R^{24a} is selected from the group consisting of substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl; and R^{24b} is hydrogen or substituted or unsubstituted C₁₋₆alkyl; or R^{24a} and R^{24b}, taken together with the carbon atom to which they are attached, form a substituted or unsubstituted C₃₋₆carbocyclyl, or a substituted or unsubstituted 3- to 10-membered heterocyclyl.

In some embodiments, R^{24a} is a non-hydrogen group selected from substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, or substituted or unsubstituted 5- to 10-membered heteroaryl. In some embodiments, R^{24a} is a substituted or unsubstituted C₂₋₄ alkyl, substituted or unsubstituted C₂₋₃alkenyl, substituted or unsubstituted C₂₋₃ alkynyl, or substituted or unsubstituted C₃ carbocyclyl. In some embodiments, R^{24a} is a substituted or unsubstituted C₁₋₄ alkyl, substituted or unsubstituted C₂₋₃alkenyl, substituted or unsubstituted C₂₋₃ alkynyl, or substituted or unsubstituted C₃ carbocyclyl. In some embodiments, R^{24a} is a non-hydrogen group substituted with fluorine. In some embodiments, R^{24a} is a non-hydrogen group substituted with one or more —OR^{A24} groups, wherein R^{A24} is hydrogen or substituted or unsubstituted C₁₋₆alkyl.

In some embodiments, R^{24a} is a substituted or unsubstituted C₁₋₆alkyl. In some embodiments, R^{24a} is unsubstituted C₁₋₆alkyl. In some embodiments, R^{24a} is substituted C₁₋₆alkyl. In some embodiments, R^{24a} is —CH₃, —CH₂CH₃, —CH(CH₃)₂, or —CF₃. In some embodiments, R^{24a} is —CH₃, —CH(CH₃)₂ or —CF₃. In some embodiments, R^{24a} is —CH₃. In some embodiments, R^{24a} is —CH(CH₃)₂. In some embodiments, R^{24a} is —CF₃. In some embodiments, R^{24a} is —CH₂OR^{A24}, —CH₂CH₂OR^{A24}, or —CH₂CH₂CH₂OR^{A24}, wherein R^{A24} is hydrogen or substituted or unsubstituted C₁₋₆alkyl.

In some embodiments, R^{24b} is hydrogen, substituted or unsubstituted C₁₋₆alkyl group, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, or substituted or unsubstituted 5- to 10-membered heteroaryl.

In some embodiments, R^{24b} is hydrogen or substituted or unsubstituted C₁₋₆alkyl. In some embodiments, R^{24b} is hydrogen or unsubstituted C₁₋₆alkyl. In some embodiments, R^{24b} is hydrogen. In some embodiments R^{24b} is unsubstituted C₁₋₆alkyl. In some embodiments, R^{24b} is —CH₃ or —CF₃. In some embodiments, R^{24b} is —CH₃.

In some embodiments, R^{24a} is —CF₃ and R^{24b} is hydrogen or C₁₋₄alkyl. In some embodiments, R^{24a} is a non-hydrogen group substituted with fluorine, and R^{24b} is —CH₃.

In some embodiments, R^{24a} is a substituted or unsubstituted C₂₋₄alkyl, substituted or unsubstituted C₂₋₃alkenyl, substituted or unsubstituted C₂₋₃ alkynyl, or substituted or unsubstituted C₃carbocyclyl; and R^{24b} is —CH₃. In some embodiments, R^{24a} is a substituted or unsubstituted C₁₋₄alkyl, substituted or unsubstituted C₂₋₃alkenyl, substituted or unsubstituted C₂₋₃ alkynyl, or substituted or unsubstituted C₃carbocyclyl, and R^{24b} is —H. In some embodiments, R^{24a} is a unsubstituted C₂₋₄alkyl, unsubstituted C₂₋₃alkenyl, or unsubstituted C₂₋₃ alkynyl, or unsubstituted C₃carbocyclyl, and R^{24b} is —H. In some embodiments, R^{24a} is unsubstituted C₂₋₄alkyl, unsubstituted C₂₋₃alkenyl, or unsubstituted C₂₋₃ alkynyl, or unsubstituted C₃carbocyclyl; and R^{24b} is —CH₃. In some embodiments, when R^{24b} is H, R^{24a} is not C₁₋₆alkyl substituted with CN.

In some embodiments, R^{24a} is a non-hydrogen group substituted with fluorine, and R^{24b} is —CH₃.

In some embodiments, R^{24a} is —CH(CH₃)₂ or —CF₃, and R^{24b} is hydrogen.

In some embodiments, R^{24a} and R^{24b}, taken together with the carbon atom to which they are attached, form a substituted or unsubstituted C₃₋₆carbocyclyl or a substituted or unsubstituted 3- to 10-membered heterocyclyl. In some embodiments, R^{24a} and R^{24b}, taken together with the carbon atom to which they are attached, form a substituted or unsubstituted C₃₋₆carbocyclyl. In some embodiments, R^{24a} and R^{24b}, taken together with the carbon atom to which they are attached, form an unsubstituted C₃₋₆carbocyclyl. In some embodiments, R^{24a} and R^{24b}, taken together with the carbon atom to which they are attached, form a cyclopropyl. In some embodiments, R^{24a} and R^{24b}, taken together with the carbon atom to which they are attached, form an unsubstituted 3- to 10-membered heterocyclyl. In some embodiments, R^{24a} and R^{24b}, taken together with the carbon atom to which they are attached, form an unsubstituted 3- to 6-membered heterocyclyl.

In some embodiments, R³ is C₁₋₃ alkyl, R^{24a} is a non-hydrogen group substituted with fluorine, and R^{24b} is —CH₃.

In some embodiments, R³ is C₁₋₃ alkyl, R^{24a} is a non-hydrogen group substituted with fluorine, and R^{24b} is hydrogen.

In some embodiments, R³ is C₁₋₃alkyl, R^{24a} is selected from the group consisting of substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, and R^{24b} is —CH₃. In some embodiments, R³ is C₁₋₃alkyl; R^{24a} is selected from the group consisting of substituted or unsubstituted C₁₋₆alkyl, unsubstituted C₂₋₆alkenyl, unsubstituted C₂₋₆alkynyl, or unsubstituted C₃₋₆carbocyclyl; and R^{24b} is —CH₃. In some embodiments, R³ is —CH₃ or CH₂CH₃; R^{24a} is selected from the group consisting of substituted or unsubstituted C₁₋₆alkyl, unsubstituted C₂₋₆alkenyl, unsubstituted C₂₋₆alkynyl, or unsubstituted C₃₋₆carbocyclyl; and and R^{24b} is —CH₃. In some embodiments, R³ is C₁₋₃ alkyl; R^{24a} is selected from a substituted or unsubstituted C14alkyl; and R^{24b} is —CH₃. In some embodiments, R³ is CH₃ or —CH₂CH₃; R^{24a} is selected from a substituted or unsubstituted C₁₋₆alkyl; and R^{24b} is —CH₃.

In some embodiments, R³ is —CH₃ or —CH₂CH₃ and at least one of R^{23a} and R^{23b} is fluorine or R^{23a} and R^{23b} are both hydrogen.

In some embodiments, R³ is —CH₃ or —CH₂CH₃ and R^{24a} is a non-hydrogen group substituted with fluorine or one or more —OR^{A24} groups, wherein R^{A24} is hydrogen or substituted or unsubstituted C₁₋₆alkyl.

In some embodiments, R³ is —CH₃ or —CH₂CH₃ and R^{24b} is —CH₃ or —CF₃. In some embodiments, R³ is —CH₃ or —CH₂CH₃; R^{24b} is —CH₃ or —CF₃; and R^{24a} is selected from the group consisting of substituted or unsubstituted C₁₋₆alkyl, unsubstituted C₂₋₆alkenyl, unsubstituted C₂₋₆alkynyl, or unsubstituted C₃₋₆carbocyclyl.

In some embodiments, R³ is —CH₃ or —CH₂CH₃, R^{24a} is —CH(CH₃)₂ or —CF₃, and R^{24b} is hydrogen.

q, r and s—as they Relate to Formulae (G-1)-(G-IVa-2)

In some embodiments, q is selected from 0, 1 or 2, and r is selected from 1 or 2. In some embodiments, q is 0. In some embodiments, q is 1. In some embodiments, q is 2. In some embodiments, r is 1. In some embodiments, r is 2. In some embodiments, q is 1 and r is 2. In some embodiments, q is 2 and r is 1. In some embodiments, q is 0 and r is 1. In some embodiments, q is 0 and r is 2. In some embodiments, q is 2 and r is 2. In some embodiments, q is 1 and r is 1.

In some embodiments, s is 1 or 2. In some embodiments, s is 1. In some embodiments, s is 2. In some embodiments, s is 3. In some embodiments, s is 4.

In some embodiments, q is 1, r is 1 and s is 1. In some embodiments, q is 1, r is 1 and s is 2. In some embodiments, q is 1, r is 1 and s is 3. In some embodiments, q is 1, r is 1 and s is 4. In some embodiments, q is 1, r is 2 and s is 1. In some embodiments, q is 1, r is 2 and s is 2. In some embodiments, q is 1, r is 2 and s is 3. In some embodiments, q is 1, r is 2 and s is 4.

In some embodiments, q is 2, r is 1 and s is 1. In some embodiments, q is 2, r is 1 and s is 2. In some embodiments, q is 2, r is 1 and s is 3. In some embodiments, q is 2, r is 1 and s is 4.

In some embodiments, q is 0, r is 1 and s is 1. In some embodiments, q is 0, r is 1 and s is 2. In some embodiments, q is 0, r is 1 and s is 3. In some embodiments, q is 0, r is 1 and s is 4.

In some embodiments, q is 0, r is 2 and s is 1. In some embodiments, q is 0, r is 2 and s is 2. In some embodiments, q is 0, r is 2 and s is 3. In some embodiments, q is 0, r is 2 and s is 4.

In some embodiments, q is 2, r is 2 and s is 1. In some embodiments, q is 2, r is 2 and s is 2. In some embodiments, q is 2, r is 2 and s is 3. In some embodiments, q is 2, r is 2, and s is 4.

In some embodiments, the compound of Formula (G-I) is any one of Compounds G-1 to G-5 in Table G-1 or pharmaceutically acceptable salts thereof. In some embodiments, the compound of Formula (G-I) is any one of Compounds G-1 to G-5 in Table G-1. In some embodiments, the compound of Formula (G-I) is a pharmaceutically acceptable salt of any one of Compounds G-1 to G-5 in Table G-1.

TABLE G-1

| Cmpd | Structure |
|---|---|
| G-1 | 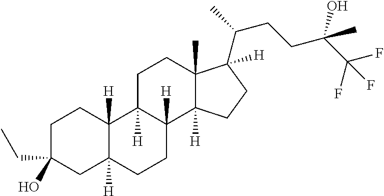 |
| G-2 | 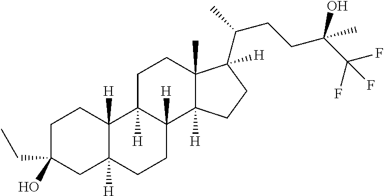 |
| G-3 | 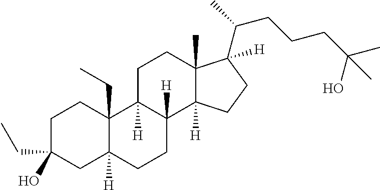 |
| G-4 | 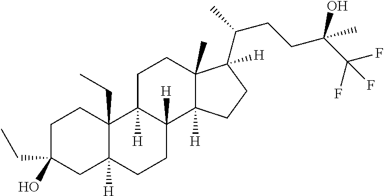 |
| G-5 | 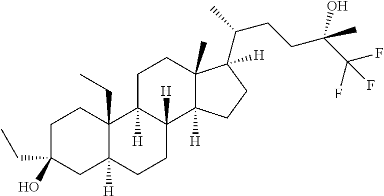 |

In one aspect, the disclosure provides a compound of Formula (H-I):

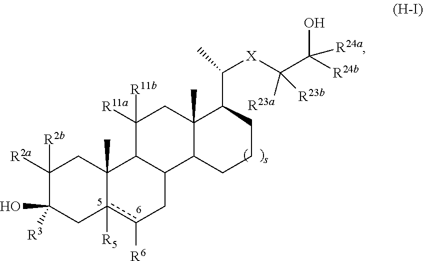

or a pharmaceutically acceptable salt thereof;
wherein:
   each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein R^{A2} is hydrogen or substituted or unsubstituted C₁₋₆alkyl;
   R³ is substituted or unsubstituted C₁₋₆alkyl, substituted or unsubstituted C₂₋₆ alkenyl, substituted or unsubstituted C₂₋₆alkynyl, or substituted or unsubstituted C₃₋₁₀ carbocyclyl;
   R⁵ is absent, hydrogen, halogen, or substituted or unsubstituted C₁₋₆alkyl;
   R⁶ is hydrogen, substituted or unsubstituted C₁₋₆alkyl, or halogen;
   R^{11a} is hydrogen or —OR^{A11}, wherein R^{A11} is hydrogen or substituted or unsubstituted alkyl, and R^{11b} is hydrogen; or R^{11a} and R^{11b} are joined to form an oxo (═O) group;
   X is —(C(R^{X})₂)ₙ— or —O—, wherein R^{X} is hydrogen or fluorine, or one R^{X} group and R^{23b} are joined to form a double bond;
   n is 1, 2 or 3;
   each of R^{23a} and R^{23b} is independently hydrogen or fluorine;
   R^{24a} is selected from the group consisting of substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, and substituted or unsubstituted heteroaryl group; and R^{24b} is hydrogen or a substituted or unsubstituted alkyl;
   or R^{24a} and R^{24b}, taken together with the carbon atom to which they are attached, form a substituted or unsubstituted C₃₋₆carbocyclyl, or substituted or unsubstituted 3- to 10-membered heterocyclyl;
   s is 1, 2 or 3; and
[symbol] represents a single or double bond, provided if the bond between C5 and C6 is a double bond, R⁵ is absent.

In some embodiments, the compound of Formula (H-I) is a compound of Formula (H-Ia):

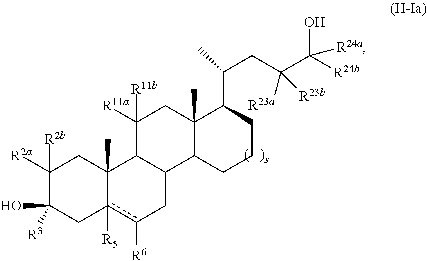

or a pharmaceutically acceptable salt thereof.

In some embodiments, the compound of Formula (H-Ia) is a compound of Formula (H-Ia-1):

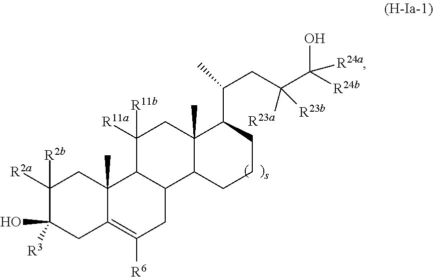

or a pharmaceutically acceptable salt thereof.

In some embodiments, the compound of Formula (H-Ia) is a compound of Formula (H-Ia-1i) or Formula (H-Ia-1ii):

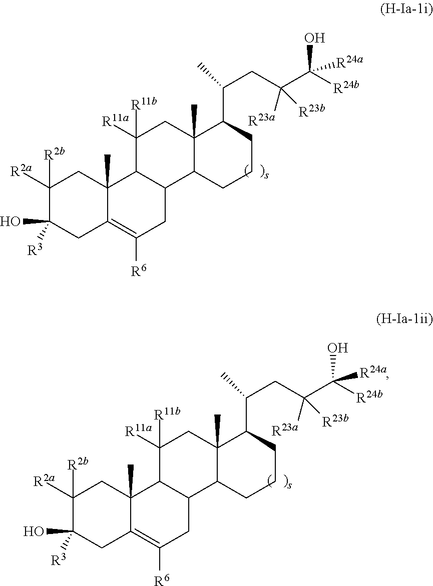

or a pharmaceutically acceptable salt thereof.

In some embodiments, the compound of Formula (H-Ia) is a compound of Formula (H-Ia-2):

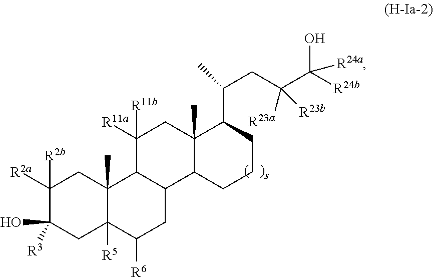

or a pharmaceutically acceptable salt thereof.

In some embodiments, the compound of Formula (H-Ia-2) is a compound of Formula (H-Ia-2i) or Formula (H-Ia-2ii):

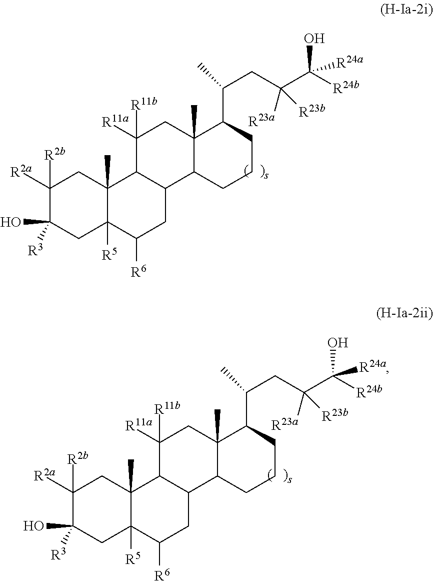

or a pharmaceutically acceptable salt thereof.

In some embodiments, the compound of Formula (H-La-2i) is a compound of Formula (H-Ia-2i-a):

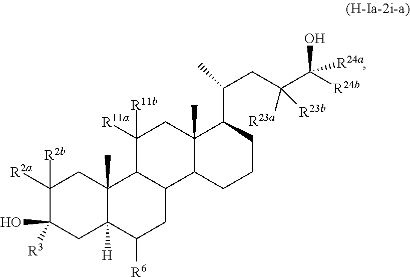

or a pharmaceutically acceptable salt thereof.

In some embodiments, the compound of Formula (H-La-2ii) is a compound of Formula (H-Ia-2ii-a):

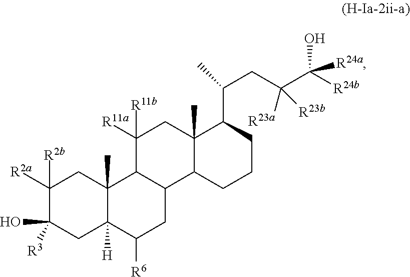

or a pharmaceutically acceptable salt thereof.

In some embodiments, the compound of Formula (H-I) is a compound of Formula (H-Ib):

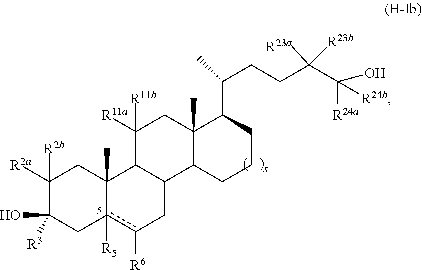

or a pharmaceutically acceptable salt thereof.

In some embodiments, the compound of Formula (H-Ib) is a compound of Formula (H-Ib-1):

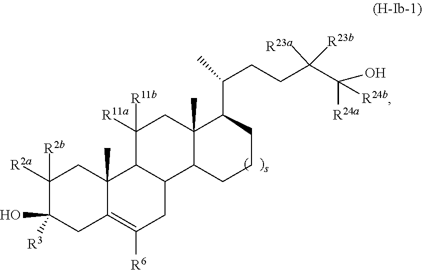

or a pharmaceutically acceptable salt thereof.

In some embodiments, the compound of Formula (H-Ib-1) is a compound of Formula (H-Ib-1i) or Formula (H-Ib-1ii):

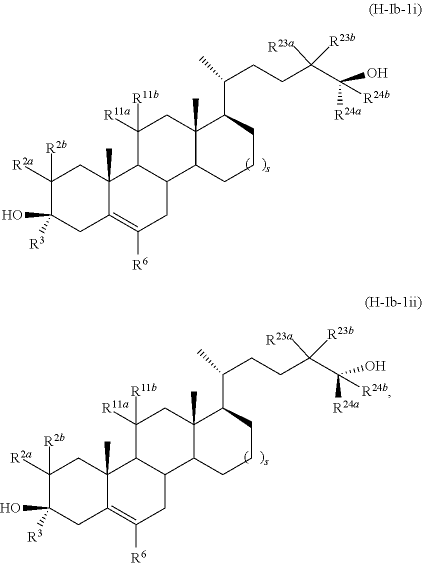

or a pharmaceutically acceptable salt thereof.

In some embodiments, the compound of Formula (H-Ib) is a compound of Formula (H-Ib-2):

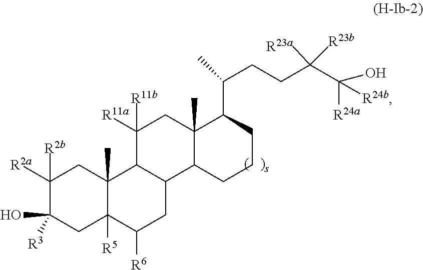

or a pharmaceutically acceptable salt thereof.

In some embodiments, the compound of Formula (H-Ib-2) is a compound of Formula (H-Ib-2i) or Formula (H-Ib-2ii):

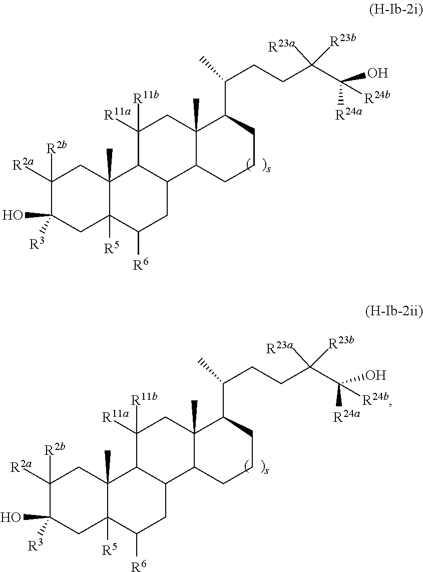

or a pharmaceutically acceptable salt thereof.

In some embodiments, the compound of Formula (H-Ib-2i) is a compound of Formula (H-Ib-2i-a):

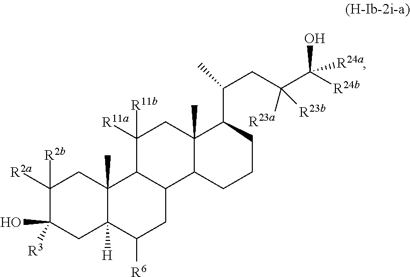

or a pharmaceutically acceptable salt thereof.

In some embodiments, the compound of Formula (H-Ib-2ii) is a compound of Formula (H-Ib-2ii-a):

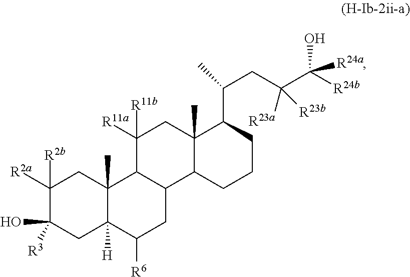

or a pharmaceutically acceptable salt thereof.
   Groups R^{2a} and R^{2b}—as they Relate to Formulae (H-I)-(H-Ib-2ii-a)

In certain embodiments, each of R^{2a} and R^{2b} is independently hydrogen, halogen, substituted or unsubstituted C₁₋₆alkyl, substituted or unsubstituted C₃₋₁₀carbocyclyl, or OR^{A2}, wherein R^{A2} is hydrogen or substituted or unsubstituted C₁₋₆alkyl. In certain embodiments, each of R^{2a} and R^{2b} is hydrogen. In certain embodiments, each of R^{2a} and R^{2b} is independently halogen, e.g., fluoro, chloro, bromo, or iodo. In certain embodiments, each of R^{2a} and R^{2b} is independently fluoro or chloro. In certain embodiments, each of R^{2a} and R^{2b} is independently substituted or unsubstituted C₁₋₆alkyl, e.g., substituted or unsubstituted C₁₋₂ alkyl, substituted or unsubstituted C₂₋₃alkyl, substituted or unsubstituted C₃₋₄ alkyl, substituted or unsubstituted C₄₋₅alkyl, or substituted or unsubstituted C₅₋₆alkyl. In certain embodiments, each of R^{2a} and R^{2b} is independently —CH₃, —CH₂CH₃, —CH₂CH₂CH₃, or cyclopropyl. In certain embodiments, each of R^{2a} and R^{2b} is independently —OR^{A2}. In certain embodiments, R^{A2} is hydrogen. In certain embodiments, R^{A2} is substituted or unsubstituted alkyl, e.g., substituted or unsubstituted C₁₋₆alkyl, substituted or unsubstituted C₁₋₂alkyl, substituted or unsubstituted C₂₋₃alkyl, substituted or unsubstituted C₃₋₄alkyl, substituted or unsubstituted C₄₋₅alkyl, or substituted or unsubstituted C₅₋₆ alkyl. In certain embodiments, R^{A2} is hydrogen, —CH₃, —CH₂CH₃, or —CH₂CH₂CH₃, i.e., to provide a group of R^{2a} or R^{2b} each independently of formula-OH, —OCH₃, —OCH₂CH₃, or —OCH₂CH₂CH₃. In certain embodiments, each of R^{2a} and R^{2b} is independently a non-hydrogen substituent. In certain embodiments, one of R^{2a} and R^{2b} is a non-hydrogen substituent in the alpha configuration. In certain embodiments, one of R^{2a} and R^{2b} is a non-hydrogen substituent in the beta configuration.

### Group R³—as it Relates to Formulae (H-I)-(H-Ib-2ii-a)

In certain embodiments, R³ is substituted or unsubstituted aliphatic, i.e., substituted or unsubstituted alkyl, substituted or unsubstituted alkenyl, substituted or unsubstituted alkynyl, or substituted or unsubstituted carbocyclyl.

In certain embodiments, R³ is substituted or unsubstituted C₁₋₆alkyl, substituted or unsubstituted C₂₋₆alkenyl, substituted or unsubstituted C₂₋₆alkynyl, or substituted or unsubstituted C₃₋₁₀carbocyclyl.

In certain embodiments, R³ is substituted or unsubstituted alkyl, e.g., substituted or unsubstituted C₁₋₆alkyl, substituted or unsubstituted C₁₋₂ alkyl, substituted or unsubstituted C₂₋₃ alkyl, substituted or unsubstituted C₃₋₄ alkyl, substituted or unsubstituted C₄₋₅alkyl, or substituted or unsubstituted C₅₋₆ alkyl. Exemplary R³ C₁₋₆alkyl groups include, but are not limited to, substituted or unsubstituted methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), n-hexyl (C₆), C₁₋₆ alkyl substituted with 1, 2, 3, 4, 5, 6, 7, 8, 9, 10, or more fluoro groups (e.g., —CF₃, —CH₂F, —CHF₂, difluoroethyl, and 2,2,2-trifluoro-1,1-dimethyl-ethyl), C₁₋₆alkyl substituted with 1, 2, 3, 4, 5, 6, 7, 8, 9, 10, or more chloro groups (e.g., —CH₂Cl, —CHCl₂), and C₁₋₆alkyl substituted with alkoxy groups (e.g., —CH₂OCH₃, —CH₂OCH₂CH₃, —CH₂O-cyclopropyl). In certain embodiments, R³ is substituted alkyl, e.g., R³ is haloalkyl, alkoxyalkyl, or aminoalkyl. In certain embodiments, R³ is Me, Et, n-Pr, n-Bu, i-Bu, fluoromethyl, chloromethyl, difluoromethyl, trifluoromethyl, trifluoroethyl, difluoroethyl, 2,2,2-trifluoro-1,1-dimethyl-ethyl, methoxymethyl, methoxyethyl, or ethoxymethyl.

In certain embodiments, R³ is substituted or unsubstituted C₁₋₆alkyl. In certain embodiments, R³ is unsubstituted C₁₋₆alkyl. In some embodiments, R³ is selected from the group consisting of CH₃, —CH₂CH₃, or —CH₂CH₂CH₃. In some embodiments, R³ is ethyl.

In certain embodiments, R³ is substituted C₁₋₆alkyl. In certain embodiments, R³ is alkyl substituted with one or more fluorine atoms, e.g., R³ is —CH₂F, —CHF₂, or —CF₃. In certain embodiments, R³ is alkyl substituted with one or more —OR^{A3} groups, wherein R^{A3} is hydrogen or substituted or unsubstituted alkyl. In certain embodiments, R³ is —CH₂OR^{A3}, e.g., wherein R^{A3} is hydrogen, —CH₃, —CH₂CH₃, or —CH₂CH₂CH₃.

In certain embodiments, R³ is —CH₃, —CH₂CH₃, —CH₂CH₂CH₃, or —CH₂OCH₃. In certain embodiments, R³ is —CH₂CH₃. In certain embodiments, R³ is —CH₂OCH₃.

In certain embodiments, R³ is substituted or unsubstituted alkenyl, e.g., substituted or unsubstituted C₂₋₆alkenyl, substituted or unsubstituted C₂₋₃alkenyl, substituted or unsubstituted C₃₋₄alkenyl, substituted or unsubstituted C₄₋₅alkenyl, or substituted or unsubstituted C₅₋₆alkenyl. In certain embodiments, R³ is ethenyl (C₂), propenyl (C₃), or butenyl (C₄), unsubstituted or substituted with one or more substituents selected from the group consisting of alkyl, halo, haloalkyl, alkoxyalkyl, or hydroxyl. In certain embodiments, R³ is ethenyl, propenyl, or butenyl, unsubstituted or substituted with alkyl, halo, haloalkyl, alkoxyalkyl, or hydroxy. In certain embodiments, R³ is ethenyl.

In certain embodiments, R³ is substituted or unsubstituted alkynyl, e.g., substituted or unsubstituted C₂₋₆alkynyl, substituted or unsubstituted C₂₋₃alkynyl, substituted or unsubstituted C₃₋₄alkynyl, substituted or unsubstituted C₄₋₅alkynyl, or substituted or unsubstituted C₅₋₆alkynyl. Exemplary substituted or unsubstituted R³ alkynyl groups include, but are not limited to, ethynyl, propynyl, or butynyl, unsubstituted or substituted with alkyl, halo, haloalkyl (e.g., CF₃), alkoxyalkyl, cycloalkyl (e.g., cyclopropyl or cyclobutyl), or hydroxyl. In certain embodiments, R³ is selected from the group consisting of trifluoroethynyl, cyclopropylethynyl, cyclobutylethynyl, and propynyl, fluoropropynyl, and chloroethynyl. In certain embodiments, R³ is ethynyl (C₂), propynyl (C₃), or butynyl (C₄), unsubstituted or substituted with one or more substituents selected from the group consisting of substituted or unsubstituted aryl, substituted or unsubstituted heteroaryl, substituted or unsubstituted carbocyclyl, and substituted or unsubstituted heterocyclyl. In certain embodiments, R³ is ethynyl (C₂), propynyl (C₃), or butynyl (C₄) substituted with substituted phenyl. In certain embodiments, the phenyl substituent is further substituted with one or more substituents selected from the group consisting of halo, alkyl, trifluoroalkyl, alkoxy, acyl, amino or amido. In certain embodiments, R³ is ethynyl (C₂), propynyl (C₃), or butynyl (C₄) substituted with substituted or unsubstituted pyrrolyl, imidazolyl, pyrazolyl, oxazoyl, thiazolyl, isoxazoyl, 1,2,3-triazolyl, 1,2,4-triazolyl, oxadiazolyl, thiadiazolyl, or tetrazolyl.

In certain embodiments, R³ is ethynyl, propynyl, or butynyl, unsubstituted or substituted with alkyl, halo, haloalkyl, alkoxyalkyl, or hydroxyl. In certain embodiments, R³ is ethynyl or propynyl, substituted with substituted or unsubstituted aryl. In certain embodiments, R³ is ethynyl or propynyl, substituted with phenyl unsubstituted or substituted with halo, alkyl, alkoxy, haloalkyl, trihaloalkyl, or acyl. In certain embodiments, R³ is ethynyl or propynyl, substituted with substituted or unsubstituted carbocyclyl. In certain embodiments, R³ is ethynyl or propynyl, substituted with substituted or unsubstituted cyclopropyl, cyclobutyl, cyclopentyl, or cyclohexyl. In certain embodiments, R³ is ethynyl or propynyl, substituted with substituted or unsubstituted heteroaryl. In certain embodiments, R³ is ethynyl or propynyl, substituted with substituted or unsubstituted pyridinyl, or pyrimidinyl. In certain embodiments, R³ is ethynyl or propynyl, substituted with substituted or unsubstituted pyrrolyl, imidazolyl, pyrazolyl, oxazoyl, thiazolyl, isoxazoyl, 1,2,3-triazolyl, 1,2,4-triazolyl, oxadiazolyl, thiadiazolyl, tetrazolyl. In certain embodiments, R³ is ethynyl or propynyl, substituted with substituted or unsubstituted heterocyclyl. In certain embodiments, R³ is ethynyl or propynyl, substituted with substituted or unsubstituted pyrrolidinyl, piperidinyl, piperazinyl, or mopholinyl. In certain embodiments, R³ is propynyl or butynyl, substituted with hydroxyl or alkoxy. In certain embodiments, R³ is propynyl or butynyl, substituted with methoxy or ethoxy. In certain embodiments, R³ is ethynyl or propynyl, substituted with chloro. In certain embodiments, R³ is ethynyl or propynyl, substituted with trifluoromethyl.

In certain embodiments, R³ is substituted or unsubstituted carbocyclyl, e.g., substituted or unsubstituted C₃₋₁₀carbocyclyl, substituted or unsubstituted C₃₋₆carbocyclyl, substituted or unsubstituted C₃₋₄ carbocyclyl, substituted or unsubstituted C₄₋₅ carbocyclyl, or substituted or unsubstituted C₅₋₆ carbocyclyl. In certain embodiments, R³ is substituted or unsubstituted cyclopropyl or substituted or unsubstituted cyclobutyl.

In certain embodiments, R³ is in the alpha configuration. In certain embodiments, R³ is in the beta configuration.

Groups R⁵ and R⁶—as they Relate to Formulae (H-I)-(H-Ib-2ii-a)

In some embodiments, R⁵ is in the alpha or beta configuration. In certain embodiments, R⁵ is in the alpha configuration. In certain embodiments, R⁵ is in the beta configuration. In some embodiments, when the bond between C5 and C6 is a double bond, R⁵ is absent. In some embodiments, the bond between C5 and C6 is a single bond and R⁵ is in the alpha configuration. In some embodiments, the bond between C5 and C6 is a single bond and R⁵ is hydrogen in the alpha configuration.

In certain embodiments, R⁵ is absent, hydrogen, halogen, or substituted or unsubstituted C₁₋₆alkyl. In some embodiments, R⁵ is halogen. In some embodiments, R⁵ is substituted C₁₋₆alkyl. In certain embodiments, R⁵ is substituted or unsubstituted alkyl, e.g., substituted or unsubstituted C₁₋₆alkyl, substituted or unsubstituted C₁₋₂alkyl, substituted or unsubstituted C₂₋₃alkyl, substituted or unsubstituted C₃₋₄alkyl, substituted or unsubstituted C₄₋₅ alkyl, or substituted or unsubstituted C₅₋₆alkyl. In some embodiments, R⁵ is substituted or unsubstituted C₁₋₆alkyl. In some embodiments, R⁵ is unsubstituted C₁₋₆alkyl. In some embodiments, R⁵ is unsubstituted C₁alkyl. In some embodiments, R⁵ is —CH₃. In some embodiments, R⁵ is hydrogen.

In certain embodiments, R⁶ is hydrogen, halogen, or substituted or unsubstituted C₁₋₆ alkyl. In certain embodiments, R⁶ is halogen, e.g., fluoro. In certain embodiments, R⁶ is substituted or unsubstituted alkyl, e.g., substituted or unsubstituted C₁₋₆alkyl, substituted or unsubstituted C₁₋₂alkyl, substituted or unsubstituted C₂₋₃alkyl, substituted or unsubstituted C₃₋₄alkyl, substituted or unsubstituted C₄₋₅alkyl, or substituted or unsubstituted C₅₋₆alkyl. In certain embodiments, R⁶ is C₁alkyl, e.g., —CH₃ or —CF₃. In certain embodiments, R⁶ is hydrogen, —CH₃, or —F. In certain embodiments, [symbol] represents a single bond and R⁶ is a non-hydrogen substituent in the alpha configuration. In certain embodiments, [symbol] represents a single bond and R⁶ is a non-hydrogen substituent in the beta configuration.

Groups R^{11a} and R^{11b}—as they Relate to Formulae (H-I)-(H-Ib-2ii-a)

In certain embodiments, R^{11a} is hydrogen or —OR^{A11}, wherein R^{A11} is hydrogen or substituted or unsubstituted C₁₋₆alkyl, and R^{11b} is hydrogen; or R^{11a} and R^{11b} are joined to form an oxo (═O) group.

In certain embodiments, R^{11a} and R^{11b} are joined to form an oxo (═O) group.

In certain embodiments, R^{11a} is —OR^{A11} and R^{11b} is hydrogen. In certain embodiments, R¹¹ª is —OR^{A11} in the alpha or beta configuration. In certain embodiments, R^{11a} is —OR^{A11} in the alpha configuration. In certain embodiments, R^{11a} is —OR^{A11} in the beta configuration. In certain embodiments, R^{A11} is hydrogen. In certain embodiments, R^{A11} is substituted or unsubstituted alkyl, e.g., substituted or unsubstituted C₁₋₆alkyl, substituted or unsubstituted C₁₋₂ alkyl, substituted or unsubstituted C₂₋₃ alkyl, substituted or unsubstituted C₃₋₄alkyl, substituted or unsubstituted C₄₋₅alkyl, or substituted or unsubstituted C₅₋₆ alkyl. In certain embodiments, R^{A11} is hydrogen, —CH₃, —CH₂CH₃, or —CH₂CH₂CH₃, i.e., to provide a group R^{11a} of formula-OH, —OCH₃, —OCH₂CH₃, or —OCH₂CH₂CH₃.

In some embodiments, each of R^{11a} and R^{11b} is hydrogen.

Groups X, n, R^{23a}, R^{23b}, R^{24a}, and R^{24b}—as they Relate to Formulae (H-I)-(H-Ib-2ii-a)

In certain embodiments X is —(C(R^{X})₂)ₙ— or —O—, wherein R^{X} is hydrogen or fluorine, or one R^{X} group and R^{23b} are joined to form a double bond.

In certain embodiments, X is —O—. In certain embodiments, X is —CH₂—. In certain embodiments, X is —CF₂—.

In certain embodiments, n is selected from 1, 2 or 3. In some embodiments, n is 1. In some embodiments, n is 2. In some embodiments, n is 3.

In some embodiments, X is —(C(R^{X})₂—, wherein R^{X} is hydrogen or fluorine. In some embodiments, X is —CH₂—. In some embodiments, X is —CF₂—.

In some embodiments, X is —(C(R^{X})₂)₂—, wherein R^{X} is hydrogen or fluorine. In some embodiments, X is —(CH₂)₂—. In some embodiments, X is —(CF₂)₂—.

In some embodiments, each of R^{23a} and R^{23b} is independently hydrogen or fluorine. In some embodiments, each of R^{23a} and R^{23b} is hydrogen. In some embodiments, at least one of R^{23a} and R^{23b} is fluorine. In some embodiments, each of R^{23a} and R^{23b} is fluorine. In certain embodiments, at least one of R^{23a} and R^{23b} is hydrogen. In certain embodiments, R^{X} and R^{23b} are joined to form a double bond, e.g., cis or trans double bond.

In some embodiments, R^{24a} is selected from the group consisting of substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl; and R^{24b} is hydrogen or substituted or unsubstituted C₁₋₆alkyl; or R^{24a} and R^{24b}, taken together with the carbon atom to which they are attached, form a substituted or unsubstituted C₃₋₆carbocyclyl or a substituted or unsubstituted 3- to 10-membered heterocyclyl.

In some embodiments, R^{24a} is selected from the group consisting of substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl; and R^{24b} is hydrogen or substituted or unsubstituted C₁₋₆alkyl; or R^{24a} and R^{24b}, taken together with the carbon atom to which they are attached, form a substituted or unsubstituted C₃₋₆carbocyclyl or a substituted or unsubstituted 3- to 10-membered heterocyclyl.

In some embodiments, R^{24a} is a non-hydrogen group selected from substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄aryl, or substituted or unsubstituted 5- to 10-membered heteroaryl. In some embodiments, R^{24a} is a substituted or unsubstituted C₂₋₄alkyl, substituted or unsubstituted C₂₋₃alkenyl, substituted or unsubstituted C₂₋₃alkynyl, or substituted or unsubstituted C₃carbocyclyl. In some embodiments, R^{24a} is a substituted or unsubstituted C₁₋₄alkyl, substituted or unsubstituted C₂₋₃alkenyl, substituted or unsubstituted C₂₋₃ alkynyl, or substituted or unsubstituted C₃carbocyclyl. In some embodiments, R^{24a} is a non-hydrogen group substituted with fluorine. In some embodiments, R^{24a} is a non-hydrogen group substituted with one or more —OR^{A24} groups, wherein R^{A24} is hydrogen or substituted or unsubstituted C₁₋₆alkyl.

In some embodiments, R^{24a} is a substituted or unsubstituted C₁₋₆alkyl. In some embodiments, R^{24a} is unsubstituted C₁₋₆alkyl. In some embodiments, R^{24a} is substituted C₁₋₆ alkyl. In some embodiments, R^{24a} is —CH₃, —CH₂CH₃, —CH(CH₃)₂, or —CF₃. In some embodiments, R^{24a} is —CH₃, —CH(CH₃)₂, or —CF₃. In some embodiments, R^{24a} is —CH₃. In some embodiments, R^{24a} is —CH(CH₃)₂. In some embodiments, R^{24a} is —CF₃. In some embodiments, R^{24a} is —CH₂OR^{A24}, —CH₂CH₂OR^{A24}, or —CH₂CH₂CH₂OR^{A24}, wherein R^{A24} is hydrogen or substituted or unsubstituted C₁₋₆alkyl.

In some embodiments, R^{24b} is hydrogen, substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, or substituted or unsubstituted 5- to 10-membered heteroaryl.

In some embodiments, R^{24b} is hydrogen or substituted or unsubstituted C₁₋₆alkyl. In some embodiments, R^{24b} is hydrogen or unsubstituted C₁₋₆alkyl. In some embodiments, R^{24b} is hydrogen. In some embodiments, R^{24b} is unsubstituted C₁₋₆alkyl. In some embodiments, R^{24b} is —CH₃ or —CF₃. In some embodiments, R^{24b} is —CH₃.

In some embodiments, R^{24a} is —CF₃ and R^{24b} is hydrogen or C₁₋₄alkyl. In some embodiments, R^{24a} is a non-hydrogen group substituted with fluorine, and R^{24b} is —CH₃.

In some embodiments, R^{24a} is substituted or unsubstituted C₂₋₄alkyl, substituted or unsubstituted C₂₋₃alkenyl, substituted or unsubstituted C₂₋₃ alkynyl, or substituted or unsubstituted C₃carbocyclyl; and R^{24b} is —CH₃. In some embodiments, R^{24a} is substituted or unsubstituted C₁₋₄alkyl, substituted or unsubstituted C₂₋₃alkenyl, substituted or unsubstituted C₂₋₃ alkynyl, or substituted or unsubstituted C₃carbocyclyl, and R^{24b} is —H. In some embodiments, R^{24a} is a unsubstituted C₂₋₄alkyl, unsubstituted C₂₋₃alkenyl, or unsubstituted C₂₋₃alkynyl, or unsubstituted C₃carbocyclyl, and R^{24b} is —H. In some embodiments, R^{24a} is unsubstituted C₂₋₄ alkyl, unsubstituted C₂₋₃alkenyl, or unsubstituted C₂₋₃ alkynyl, or unsubstituted C₃carbocyclyl; and R^{24b} is —CH₃. In some embodiments, when R^{24b} is H, R^{24a} is not C₁₋₆alkyl substituted with CN.

In some embodiments, R^{24a} is a non-hydrogen group substituted with fluorine and R^{24b} is —CH₃.

In some embodiments, R^{24a} is —CH(CH₃)₂ or —CF₃ and R^{24b} is hydrogen.

In some embodiments, R^{24a} and R^{24b}, taken together with the carbon atom to which they are attached, form a substituted or unsubstituted C₃₋₆carbocyclyl or a substituted or unsubstituted 3- to 10-membered heterocyclyl. In some embodiments, R^{24a} and R^{24b}, taken together with the carbon atom to which they are attached, form a substituted or unsubstituted C₃₋₆carbocyclyl. In some embodiments, R^{24a} and R^{24b}, taken together with the carbon atom to which they are attached, form an unsubstituted C₃₋₆carbocyclyl. In some embodiments, R^{24a} and R^{24b}, taken together with the carbon atom to which they are attached, form a cyclopropyl. In some embodiments, R^{24a} and R^{24b}, taken together with the carbon atom to which they are attached, form an unsubstituted 3- to 10-membered heterocyclyl. In some embodiments, R^{24a} and R^{24b}, taken together with the carbon atom to which they are attached, form an unsubstituted 3- to 6-membered heterocyclyl.

In some embodiments, R³ is C₁₋₃ alkyl, R^{24a} is a non-hydrogen group substituted with fluorine, and R^{24b} is —CH₃.

In some embodiments, R³ is C₁₋₃ alkyl, R^{24a} is a non-hydrogen group substituted with fluorine, and R^{24b} is hydrogen.

In some embodiments, R³ is C₁₋₃ alkyl, R^{24a} is selected from the group consisting of substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, and R^{24b} is —CH₃. In some embodiments, R³ is C₁₋₃ alkyl; R^{24a} is selected from the group consisting of substituted or unsubstituted C₁₋₆alkyl, unsubstituted C₂₋₆alkenyl, unsubstituted C₂₋₆alkynyl, or unsubstituted C₃₋₆carbocyclyl; and R^{24b} is —CH₃. In some embodiments, R³ is —CH₃ or CH₂CH₃; R^{24a} is selected from the group consisting of substituted or unsubstituted C₁₋₆alkyl, unsubstituted C₂₋₆alkenyl, unsubstituted C₂₋₆alkynyl, or unsubstituted C₃₋₆carbocyclyl; and R^{24b} is —CH₃. In some embodiments, R³ is C₁₋₃ alkyl; R^{24a} is selected from a substituted or unsubstituted C₁₋₆alkyl; and R^{24b} is —CH₃. In some embodiments, R³ is CH₃ or —CH₂CH₃; R^{24a} is substituted or unsubstituted C₁₋₆alkyl; and R^{24b} is —CH₃.

In some embodiments, R³ is —CH₃ or —CH₂CH₃ and at least one of R^{23a} and R^{23b} is fluorine or R^{23a} and R^{23b} are both hydrogen.

In some embodiments, R³ is —CH₃ or —CH₂CH₃ and R^{24a} is a non-hydrogen group substituted with fluorine or one or more —OR^{A24} groups, wherein R^{A24} is hydrogen or substituted or unsubstituted C₁₋₆alkyl.

In some embodiments, R³ is —CH₃ or —CH₂CH₃ and R^{24b} is —CH₃ or —CF₃. In some embodiments, R³ is —CH₃ or —CH₂CH₃; R^{24b} is —CH₃ or —CF₃; and R^{24a} is selected from the group consisting of substituted or unsubstituted C₁₋₆alkyl, unsubstituted C₂₋₆alkenyl, unsubstituted C₂₋₆alkynyl, and unsubstituted C₃₋₆carbocyclyl.

In some embodiments, R³ is —CH₃ or —CH₂CH₃; R^{24a} is —CH(CH₃)₂ or —CF₃; and R^{24b} is hydrogen.

s—as its Relates to Formulae (H-I)-(H-Ib-2ii-a)

In some embodiments, s is 1, 2 or 3. In some embodiments, s is 1 or 2. In some embodiments, s is 1. In some embodiments, s is 2. In some embodiments, s is 3.

In some embodiments, the compound of Formula (H-I) is any one of Compounds H-1 to H-11 in Table H-1 or pharmaceutically acceptable salts thereof. In some embodiments, the compound of Formula (H-I) is any one of Compounds H-1 to H-11 in Table H-1. In some embodiments, the compound of Formula (H-I) is a pharmaceutically acceptable salt of any one of Compounds H-1 to H-11 in Table H-1.

TABLE H-1

| Com- | |
|---|---|
| pound | Structure |
| H-1 | 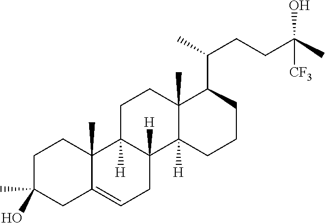 |
| H-2 | 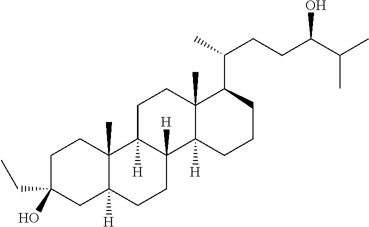 |
| H-3 | 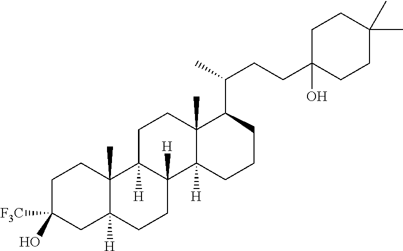 |
| H-4 | 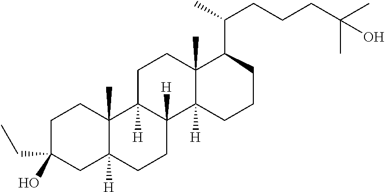 |
| H-5 | 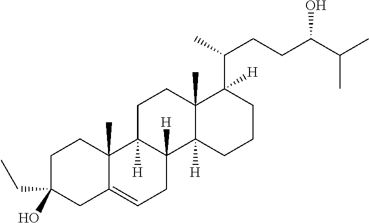 |
| H-6 | 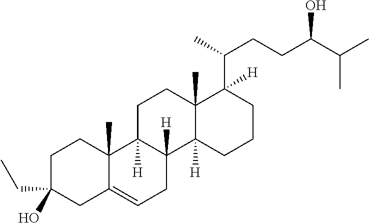 |
| H-7 | 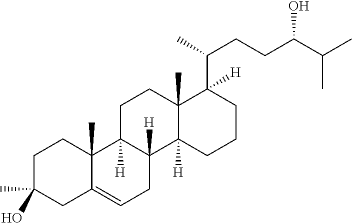 |
| H-8 | 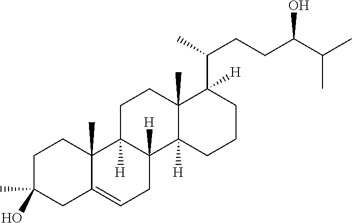 |
| H-9 | 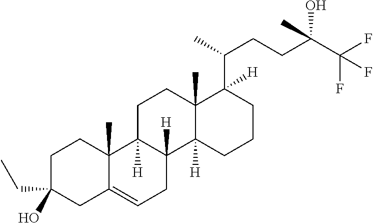 |
| H-10 | 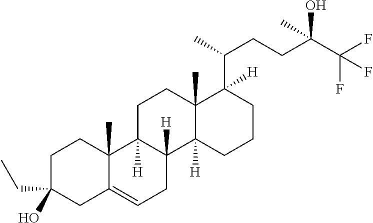 |
| H-11 | 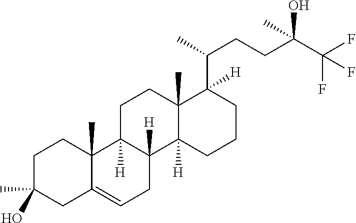 |

### Pharmaceutical Compositions

In another aspect, the disclosure provides a pharmaceutical composition comprising a pharmaceutically acceptable carrier and an effective amount of a compound described herein (e.g., a compound of Formulae (A-I), (B-I), (C-I), (D-I), (E-I), (F-I), (G-I), or (H-I), or a pharmaceutically acceptable salt thereof).

When employed as pharmaceuticals, the compounds provided herein are typically administered in the form of a pharmaceutical composition. Such compositions can be prepared in a manner well-known in the pharmaceutical arts and comprise at least one active compound.

In one embodiment, with respect to the pharmaceutical composition, the carrier is a parenteral carrier, oral or topical carrier.

The present disclosure also relates to a compound described herein (e.g., a compound of Formulae (A-I), (B-I), (C-I), (D-I), (E-I), (F-I), (G-I), or (H-I), or a pharmaceutically acceptable salt or pharmaceutical composition thereof) for use as a pharmaceutical or a medicament.

Generally, the compounds provided herein are administered in a therapeutically effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

In one aspect, the disclosure provides a pharmaceutical composition comprising: (a) a compound disclosed herein; (b) an anti-amyloid beta antibody or antigen-binding fragment thereof; and (c) a pharmaceutically acceptable carrier.

In some embodiments, the anti-amyloid beta antibody or antigen-binding fragment thereof is selected from the group consisting of bapineuzumab, solanezumab, gantenerumab, crenezumab, ponezumab, lecanemab (BAN2401), aducanumab, and antigen-binding fragments thereof.

In some embodiments, the anti-amyloid beta antibody or antigen-binding fragment thereof comprises: (a) a heavy chain variable domain (VH) comprising (i) HCDR1 comprising the amino acid sequence of SEQ ID NO: 1, (ii) HCDR2 comprising the amino acid sequence of SEQ ID NO: 2, and (iii) HCDR³ comprising the amino acid sequence of SEQ ID NO: 3; and (b) a light chain variable domain (VL) comprising (i) LCDR1 comprising the amino acid sequence of SEQ ID NO: 4, (ii) LCDR2 comprising the amino acid sequence of SEQ ID NO: 5, and (iii) LCDR³ comprising the amino acid sequence of SEQ ID NO: 6.

In some embodiments, the anti-amyloid beta antibody or antigen-binding fragment thereof comprises a heavy chain variable domain (VH) comprising the amino acid sequence of SEQ ID NO:7.

In some embodiments, the anti-amyloid beta antibody or antigen-binding fragment thereof comprises a light chain variable domain (VL) comprising the amino acid sequence of SEQ ID NO:8.

In some embodiments, the anti-amyloid beta antibody or antigen-binding fragment thereof comprises a heavy chain variable domain (VH) comprising the amino acid sequence of SEQ ID NO:7; and a light chain variable domain (VL) comprising the amino acid sequence of SEQ ID NO:8.

In some embodiments, the anti-amyloid beta antibody comprises a heavy chain comprising the amino acid sequence of SEQ ID NO:9.

In some embodiments, the anti-amyloid beta antibody comprises a light chain comprising the amino acid sequence of SEQ ID NO:10.

In some embodiments, the anti-amyloid beta antibody comprises a heavy chain comprising the amino acid sequence of SEQ ID NO:9; and a light chain comprising the amino acid sequence of SEQ ID NO:10.

In some embodiments, the anti-amyloid beta antibody or antigen-binding fragment thereof comprises: (a) a heavy chain variable domain (VH) comprising (i) HCDR1 comprising the amino acid sequence of SEQ ID NO: 11, (ii) HCDR2 comprising the amino acid sequence of SEQ ID NO: 12, and (iii) HCDR³ comprising the amino acid sequence of SEQ ID NO: 13; and (b) a light chain variable domain (VL) comprising (i) LCDR1 comprising the amino acid sequence of SEQ ID NO: 14, (ii) LCDR2 comprising the amino acid sequence of SEQ ID NO: 15, and (iii) LCDR³ comprising the amino acid sequence of SEQ ID NO: 16.

In some embodiments, the anti-amyloid beta antibody or antigen-binding fragment thereof comprises a heavy chain variable domain (VH) comprising the amino acid sequence of SEQ ID NO:17.

In some embodiments, the anti-amyloid beta antibody or antigen-binding fragment thereof comprises a light chain variable domain (VL) comprising the amino acid sequence of SEQ ID NO:18.

In some embodiments, the anti-amyloid beta antibody or antigen-binding fragment thereof comprises a heavy chain variable domain (VH) comprising the amino acid sequence of SEQ ID NO:17; and a light chain variable domain (VL) comprising the amino acid sequence of SEQ ID NO:18.

In some embodiments, the anti-amyloid beta antibody comprises a heavy chain comprising the amino acid sequence of SEQ ID NO: 19.

In some embodiments, the anti-amyloid beta antibody comprises a light chain comprising the amino acid sequence of SEQ ID NO:20.

In some embodiments, the anti-amyloid beta antibody comprises a heavy chain comprising the amino acid sequence of SEQ ID NO: 19; and a light chain comprising the amino acid sequence of SEQ ID NO:20.

In some embodiments, the anti-amyloid beta antibody or antigen-binding fragment thereof comprises: (a) a heavy chain variable domain (VH) comprising (i) HCDR1 comprising the amino acid sequence of SEQ ID NO: 21, (ii) HCDR2 comprising the amino acid sequence of SEQ ID NO: 22, and (iii) HCDR³ comprising the amino acid sequence of SEQ ID NO: 23; and (b) a light chain variable domain (VL) comprising (i) LCDR1 comprising the amino acid sequence of SEQ ID NO: 24, (ii) LCDR2 comprising the amino acid sequence of SEQ ID NO: 25, and (iii) LCDR³ comprising the amino acid sequence of SEQ ID NO: 26.

In some embodiments, the anti-amyloid beta antibody or antigen-binding fragment thereof comprises a heavy chain variable domain (VH) comprising the amino acid sequence of SEQ ID NO:27.

In some embodiments, the anti-amyloid beta antibody or antigen-binding fragment thereof comprises a light chain variable domain (VL) comprising the amino acid sequence of SEQ ID NO:28.

In some embodiments, the anti-amyloid beta antibody or antigen-binding fragment thereof comprises a heavy chain variable domain (VH) comprising the amino acid sequence of SEQ ID NO:27; and a light chain variable domain (VL) comprising the amino acid sequence of SEQ ID NO:28.

In some embodiments, the anti-amyloid beta antibody comprises a heavy chain comprising the amino acid sequence of SEQ ID NO:29.

In some embodiments, the anti-amyloid beta antibody comprises a light chain comprising the amino acid sequence of SEQ ID NO:30.

In some embodiments, the anti-amyloid beta antibody comprises a heavy chain comprising the amino acid sequence of SEQ ID NO:29; and a light chain comprising the amino acid sequence of SEQ ID NO:30.

In some embodiments, the anti-amyloid beta antibody or antigen-binding fragment thereof comprises: (a) a heavy chain variable domain (VH) comprising (i) HCDR1 comprising the amino acid sequence of SEQ ID NO: 31, (ii) HCDR2 comprising the amino acid sequence of SEQ ID NO: 32, and (iii) HCDR³ comprising the amino acid sequence of SEQ ID NO: 33; and (b) a light chain variable domain (VL) comprising (i) LCDR1 comprising the amino acid sequence of SEQ ID NO: 34, (ii) LCDR2 comprising the amino acid sequence of SEQ ID NO: 35, and (iii) LCDR³ comprising the amino acid sequence of SEQ ID NO: 36.

In some embodiments, the anti-amyloid beta antibody or antigen-binding fragment thereof comprises a heavy chain variable domain (VH) comprising the amino acid sequence of SEQ ID NO:37.

In some embodiments, the anti-amyloid beta antibody or antigen-binding fragment thereof comprises a light chain variable domain (VL) comprising the amino acid sequence of SEQ ID NO:38.

In some embodiments, the anti-amyloid beta antibody or antigen-binding fragment thereof comprises a heavy chain variable domain (VH) comprising the amino acid sequence of SEQ ID NO:37; and a light chain variable domain (VL) comprising the amino acid sequence of SEQ ID NO:38.

In some embodiments, the anti-amyloid beta antibody comprises a heavy chain comprising the amino acid sequence of SEQ ID NO:39.

In some embodiments, the anti-amyloid beta antibody comprises a light chain comprising the amino acid sequence of SEQ ID NO:40.

In some embodiments, the anti-amyloid beta antibody comprises a heavy chain comprising the amino acid sequence of SEQ ID NO:39; and a light chain comprising the amino acid sequence of SEQ ID NO:40.

In some embodiments, the anti-amyloid beta antibody or antigen-binding fragment thereof comprises: (a) a heavy chain variable domain (VH) comprising (i) HCDR1 comprising the amino acid sequence of SEQ ID NO: 41, (ii) HCDR2 comprising the amino acid sequence of SEQ ID NO: 42, and (iii) HCDR³ comprising the amino acid sequence of SEQ ID NO: 43; and (b) a light chain variable domain (VL) comprising (i) LCDR1 comprising the amino acid sequence of SEQ ID NO: 44, (ii) LCDR2 comprising the amino acid sequence of SEQ ID NO: 45, and (iii) LCDR³ comprising the amino acid sequence of SEQ ID NO: 46.

In some embodiments, the anti-amyloid beta antibody or antigen-binding fragment thereof comprises a heavy chain variable domain (VH) comprising the amino acid sequence of SEQ ID NO:47.

In some embodiments, the anti-amyloid beta antibody or antigen-binding fragment thereof comprises a light chain variable domain (VL) comprising the amino acid sequence of SEQ ID NO:48.

In some embodiments, the anti-amyloid beta antibody or antigen-binding fragment thereof comprises a heavy chain variable domain (VH) comprising the amino acid sequence of SEQ ID NO:47; and a light chain variable domain (VL) comprising the amino acid sequence of SEQ ID NO:48.

In some embodiments, the anti-amyloid beta antibody comprises a heavy chain comprising the amino acid sequence of SEQ ID NO:49.

In some embodiments, the anti-amyloid beta antibody comprises a light chain comprising the amino acid sequence of SEQ ID NO:50.

In some embodiments, the anti-amyloid beta antibody comprises a heavy chain comprising the amino acid sequence of SEQ ID NO:49; and a light chain comprising the amino acid sequence of SEQ ID NO:50.

In some embodiments, the anti-amyloid beta antibody or antigen-binding fragment thereof comprises: (a) a heavy chain variable domain (VH) comprising (i) HCDR1 comprising the amino acid sequence of SEQ ID NO: 51, (ii) HCDR2 comprising the amino acid sequence of SEQ ID NO: 52, and (iii) HCDR³ comprising the amino acid sequence of SEQ ID NO: 53; and (b) a light chain variable domain (VL) comprising (i) LCDR1 comprising the amino acid sequence of SEQ ID NO: 54, (ii) LCDR2 comprising the amino acid sequence of SEQ ID NO: 55, and (iii) LCDR³ comprising the amino acid sequence of SEQ ID NO: 56.

In some embodiments, the anti-amyloid beta antibody or antigen-binding fragment thereof comprises a heavy chain variable domain (VH) comprising the amino acid sequence of SEQ ID NO:57.

In some embodiments, the anti-amyloid beta antibody or antigen-binding fragment thereof comprises a light chain variable domain (VL) comprising the amino acid sequence of SEQ ID NO:58.

In some embodiments, the anti-amyloid beta antibody or antigen-binding fragment thereof comprises a heavy chain variable domain (VH) comprising the amino acid sequence of SEQ ID NO:57; and a light chain variable domain (VL) comprising the amino acid sequence of SEQ ID NO:58.

In some embodiments, the anti-amyloid beta antibody comprises a heavy chain comprising the amino acid sequence of SEQ ID NO:59.

In some embodiments, the anti-amyloid beta antibody comprises a light chain comprising the amino acid sequence of SEQ ID NO:60.

In some embodiments, the anti-amyloid beta antibody comprises a heavy chain comprising the amino acid sequence of SEQ ID NO:59; and a light chain comprising the amino acid sequence of SEQ ID NO:60.

In some embodiments, the anti-amyloid beta antibody or antigen-binding fragment thereof comprises: (a) a heavy chain variable domain (VH) comprising (i) HCDR1 comprising the amino acid sequence of SEQ ID NO: 61, (ii) HCDR2 comprising the amino acid sequence of SEQ ID NO: 62, and (iii) HCDR³ comprising the amino acid sequence of SEQ ID NO: 63; and (b) a light chain variable domain (VL) comprising (i) LCDR1 comprising the amino acid sequence of SEQ ID NO: 64, (ii) LCDR2 comprising the amino acid sequence of SEQ ID NO: 65, and (iii) LCDR³ comprising the amino acid sequence of SEQ ID NO: 66.

In some embodiments, the anti-amyloid beta antibody or antigen-binding fragment thereof comprises a heavy chain variable domain (VH) comprising the amino acid sequence of SEQ ID NO:67.

In some embodiments, the anti-amyloid beta antibody or antigen-binding fragment thereof comprises a light chain variable domain (VL) comprising the amino acid sequence of SEQ ID NO:68.

In some embodiments, the anti-amyloid beta antibody or antigen-binding fragment thereof comprises a heavy chain variable domain (VH) comprising the amino acid sequence of SEQ ID NO:67; and a light chain variable domain (VL) comprising the amino acid sequence of SEQ ID NO:68.

In some embodiments, the anti-amyloid beta antibody comprises a heavy chain comprising the amino acid sequence of SEQ ID NO:69.

In some embodiments, the anti-amyloid beta antibody comprises a light chain comprising the amino acid sequence of SEQ ID NO: 70.

In some embodiments, the anti-amyloid beta antibody comprises a heavy chain comprising the amino acid sequence of SEQ ID NO:69; and a light chain comprising the amino acid sequence of SEQ ID NO:70.

In some embodiments, the anti-amyloid beta antibody or antigen-binding fragment thereof comprises a human IgG1, IgG2, IgG3, or IgG4 constant domain. In some embodiments, the anti-amyloid beta antibody or antigen-binding fragment thereof comprises a human IgG1 constant region.

In some embodiments, the anti-amyloid beta antibody or antigen-binding fragment thereof is a human anti-amyloid beta antibody or antigen-binding fragment thereof.

In some embodiments, the antigen-binding fragment is a single chain antibody, Fv, Fab, Fab′, F(ab′)₂, Fd, single chain Fv molecule (scFv), bispecific single chain Fv dimer, diabody, domain-deleted antibody or single domain antibody (dAb).

In some embodiments, the anti-amyloid beta antibody is aducanumab. In some embodiments, the anti-amyloid beta antibody is bapineuzumab. In some embodiments, the anti-amyloid beta antibody is solanezumab. In some embodiments, the anti-amyloid beta antibody is gantenerumab. In some embodiments, the anti-amyloid beta antibody is crenezumab. In some embodiments, the anti-amyloid beta antibody is ponezumab. In some embodiments, the anti-amyloid beta antibody is lecanemab (BAN2401).

In some embodiments, the anti-amyloid beta antibody or antigen binding fragment thereof binds to the same conformational epitope of amyloid beta to which aducanumab binds. In some embodiments, the anti-amyloid beta antibody or antigen binding fragment thereof competitively inhibits binding of aducanumab to amyloid beta.

In some embodiments, the anti-amyloid beta antibody or antigen binding fragment thereof binds to the same conformational epitope of amyloid beta to which bapineuzumab binds. In some embodiments, the anti-amyloid beta antibody or antigen binding fragment thereof competitively inhibits binding of bapineuzumab to amyloid beta.

In some embodiments, the anti-amyloid beta antibody or antigen binding fragment thereof binds to the same conformational epitope of amyloid beta to which solanezumab binds. In some embodiments, the anti-amyloid beta antibody or antigen binding fragment thereof competitively inhibits binding of solanezumab to amyloid beta.

In some embodiments, the anti-amyloid beta antibody or antigen binding fragment thereof binds to the same conformational epitope of amyloid beta to which gantenerumab binds. In some embodiments, the anti-amyloid beta antibody or antigen binding fragment thereof competitively inhibits binding of gantenerumab to amyloid beta.

In some embodiments, the anti-amyloid beta antibody or antigen binding fragment thereof binds to the same conformational epitope of amyloid beta to which crenezumab binds. In some embodiments, the anti-amyloid beta antibody or antigen binding fragment thereof competitively inhibits binding of crenezumab to amyloid beta.

In some embodiments, the anti-amyloid beta antibody or antigen binding fragment thereof binds to the same conformational epitope of amyloid beta to which ponezumab binds.

In some embodiments, the anti-amyloid beta antibody or antigen binding fragment thereof competitively inhibits binding of ponezumab to amyloid beta.

In some embodiments, the anti-amyloid beta antibody or antigen binding fragment thereof binds to the same conformational epitope of amyloid beta to which lecanemab (BAN2401) binds. In some embodiments, the anti-amyloid beta antibody or antigen binding fragment thereof competitively inhibits binding of lecanemab (BAN2401) to amyloid beta.

In some embodiments, the anti-amyloid beta antibody or antigen binding fragment thereof binds to amyloid beta plaques, parenchymal amyloid, cerebrovascular amyloid, or diffuse amyloid beta deposits.

The anti-amyloid beta antibody or antigen-binding fragment thereof useful in the compositions disclosed herein may comprise any of the complementarity determining regions (CDRs) (i.e., HCDRs, LCDRs), VH, VL, heavy chain or light chain sequences set forth in Tables A-G. Tables A-G provide the details of the VH, VL and of the various anti-amyloid beta antibodies.

TABLE A
Aducanumab (HCDR1-3 and LCDR1-3 based on Kabat nomenclature)

| SEQ ID NO | Description | Amino Acid Sequence |
|---|---|---|
| SEQ ID NO: 1 | HCDR1 | SYGMH |
| SEQ ID NO: 2 | HCDR2 | VIWEDGMKYYTDSVKG |
| SEQ ID NO: 3 | HCDR3 | DRGIGARRGPYYMDV |
| SEQ ID NO: 4 | LCDR1 | RASQSISSYLN |
| SEQ ID NO: 5 | LCDR2 | AASSLQS |
| SEQ ID NO: 6 | LCDR3 | QQSYSTPLI |
| SEQ ID NO: 7 | VH | QVQLVESGGGVVQPGRSLRLSCAASGFAFSSYGMH |
| | | WVRQAPGKGLEWVAVIWFDGTKKYYTDSVKGRFTI |
| | | SRDNSKNTLYLQMNTLRAEDTAVYYCARDRGIGAR |
| | | RGPYYMDVWGKGTTVYVSS |
| SEQ ID NO: 8 | VL | DIQMTQSPSSLSASVGDRVTITCRASQSISSYLNWYQ |
| | | QKPGKAPKLLIYAASSLQSGVPSRFSGSGSGTDFTLTI |
| | | SSLQPEDFATYYCQQSYSTPLTFGGGTKVEIKR |
| SEQ ID NO: 9 | Heavy chain | QVQLVESGGGVVQPGRSLRLSCAASGFAFSSYGMH |
| | (H-Gamma- | WVRQAPGKGLEWVAVIWFDGTKKYYTDSVKGRFTI |
| | 1) | SRDNSKNTLYLQMNTLRAEDTAVYYCARDRGIGAR |
| | | RGPYYMDVWGKGTTVTVSSASTKGPSVFPLAPSSKS |
| | | TSGGTAALGCLVKDYFPEPVTVSWNSGALTSGVHTF |
| | | PAVLQSSGLYSLSSVVTVPSSSLGTQTYICNVNHKPS |
| | | NTKVDKRVEPKSCDKTHTCPPCPAPELLGGPSVFLFP |
| | | PKPKDTLMISRTPEVTCVVVDVSHEDPEVKFNWYVD |
| | | GVEVHNAKTKPREEQYNSTYRVVSVLTVLHQDWLN |
| | | GKEYKCKVSNKALPAPIEKTISKAKGQPREPQVYTLP |
| | | PSREEMTKNQVSLTCLVKGFYPSDIAVEWESNGQPE |
| | | NNYKTTPPVLDSDGSFFLYSKLTVDKSRWQQGNVFS |
| | | CSVMHEALHNHYTQKSLSLSPG |
| SEQ ID NO: 10 | Light chain | DIQMTQSPSSLSASVGDRVTITCRASQSISSYLNWYQ |
| | (L-Kappa) | QKPGKAPKLLIYAASSLQSGVPSRFSGSGSGTDFTLTI |
| | | SSLQPEDFATYYCQQSYSTPLTFGGGTKVEIKRTVAA |
| | | PSVFIFPPSDEQLKSGTASVVCLLNNFYPREAKVQWK |
| | | VDNALQSGNSQESVTEQDSKDSTYSLSSTLTLSKADY |
| | | EKHKVYACEVTHQGLSSPVTKSFNRGEC |

TABLE B
Solanezumab (HCDR1-3 and LCDR1-3 based on IMGT nomenclature)

| SEQ ID NO | Description | Amino Acid Sequence |
|---|---|---|
| SEQ ID NO: 11 | HCDR1 | GFTFSRYS |
| SEQ ID NO: 12 | HCDR2 | INSVGNST |
| SEQ ID NO: 13 | HCDR3 | ASGDY |
| SEQ ID NO: 14 | LCDR1 | QSLIYSDGNAY |
| SEQ ID NO: 15 | LCDR2 | KVS |
| SEQ ID NO: 16 | LCDR3 | SQSTHVPWT |
| SEQ ID NO: 17 | VH | EVQLVESGGGLVQPGGSLRLSCAASGFTFSRYSMS |
| | | WVRQAPGKGLELVAQINSVGNSTYYPDTVKGRFTI |
| | | SRDNAKNTLYLQMNSLRAEDTAVYYCA |
| SEQ ID NO: 18 | VL | DVVMTQSPLSLPVTLGQPASISCRSSQSLIYSDGNAY |
| | | LHWFLQKPGQSPRLLIYKVSNRFSGVPDRFSGSGSG |
| | | TDFTLKISRVEAEDVGVYYCSQSTHVP |
| SEQ ID NO: 19 | Heavy | EVQLVESGGGLVQPGGSLRLSCAASGFTFSRYSMS |
| | chain | WVRQAPGKGLELVAQINSVGNSTYYPDTVKGRFTI |
| | (H-Gamma- | SRDNAKNTLYLQMNSLRAEDTAVYYCASGDYWGQ |
| | 1) | GTLVTVSSASTKGPSVFPLAPSSKSTSGGTAALGCL |
| | | VKDYFPEPVTVSWNSGALTSGVHTFPAVLQSSGLYS |
| | | LSSVVTVPSSSLGTQTYICNVNHKPSNTKVDKKVEP |
| | | KSCDKTHTCPPCPAPELLGGPSVFLFPPKPKDTLMIS |
| | | RTPEVTCVVVDVSHEDPEVKFNWYVDGVEVHNAK |
| | | TKPREEQYNSTYRVVSVLTVLHQDWLNGKEYKCK |
| | | VSNKALPAPIEKTISKAKGQPREPQVYTLPPSRDELT |
| | | KNQVSLTCLVKGFYPSDIAVEWESNGQPENNYKTT |
| | | PPVLDSDGSFFLYSKLTVDKSRWQQGNVFSCSVMH |
| | | EALHNHYTQKSLSLSPGK |
| SEQ ID NO: 20 | Light chain | DVVMTQSPLSLPVTLGQPASISCRSSQSLIYSDGNAY |
| | (L-Kappa) | LHWFLQKPGQSPRLLIYKVSNRFSGVPDRFSGSGSG |
| | | TDFTLKISRVEAEDVGVYYCSQSTHVPWTFGQGTK |
| | | VEIKRTVAAPSVFIFPPSDEQLKSGTASVVCLLNNFY |
| | | PREAKVQWKVDNALQSGNSQESVTEQDSKDSTYSL |
| | | SSTLTLSKADYEKHKVYACEVTHQGLSSPVTKSFNR |
| | | GEC |

TABLE C
Crenezumab (HCDR1-3 and LCDR1-3 based on IMGT nomenclature)

| SEQ ID NO | Description | Amino Acid Sequence |
|---|---|---|
| SEQ ID NO: 21 | HCDR1 | GFTFSSYG |
| SEQ ID NO: 22 | HCDR2 | INSNGGST |
| SEQ ID NO: 23 | HCDR3 | ASGDY |
| SEQ ID NO: 24 | LCDR1 | QSLVYSNGDTY |
| SEQ ID NO: 25 | LCDR2 | KVS |
| SEQ ID NO: 26 | LCDR3 | SQSTHVPWT |
| SEQ ID NO: 27 | VH | EVQLVESGGGLVQPGGSLRLSCAASGFTFSSYGMS |
| | | WVRQAPGKGLELVASINSNGGSTYYPDSVKGRFTIS |
| | | RDNAKNSLYLQMNSLRAEDTAVYYCA |
| SEQ ID NO: 28 | VL | DIVMTQSPLSLPVTPGEPASISCRSSQSLVYSNGDTY |
| | | LHWYLQKPGQSPQLLIYKVSNRFSGVPDRFSGSGSG |
| | | TDFTLKISRVEAEDVGVYYCSQSTHV |
| SEQ ID NO: 29 | Heavy | EVQLVESGGGLVQPGGSLRLSCAASGFTFSSYGMS |
| | chain | WVRQAPGKGLELVASINSNGGSTYYPDSVKGRFTIS |
| | H-Gamma- | RDNAKNSLYLQMNSLRAEDTAVYYCASGDYWGQ |
| | 4 | GTTVTVSSASTKGPSVFPLAPCSRSTSESTAALGCLV |
| | | KDYFPEPVTVSWNSGALTSGVHTFPAVLQSSGLYSL |
| | | SSVCHVTVPSSSLGTKTYTCNVDHKPSNTKVDKRV |
| | | ESKYGPPCPPCPAPEFLGGPSVFLFPPKPKDTLMISRT |
| | | PEVTCVVVDVSQEDPEVQFNWYVDGVEVHNAKTK |
| | | PREEQFNSTYRVVSVLTVLHQDWLNGKEYKCKVSN |
| | | KGLPSSIEKTISKAKGQPREPQVYTLPPSQEEMTKNQ |
| | | VSLTCLVKGFYPSDIAVEWESNGQPENNYKTTPPVL |
| | | DSDGSFFLYSRLTVDKSRWQEGNVFSCSVMHEALH |
| | | NHYTQKSLSLSLG |
| SEQ ID NO: 30 | Light chain | DIVMTQSPLSLPVTPGEPASISCRSSQSLVYSNGDTY |
| | L-Kappa | LHWYLQKPGQSPQLLIYKVSNRFSGVPDRFSGSGSG |
| | | TDFTLKISRVEAEDVGVYYCSQSTHVPWTFGQGTK |
| | | VEIKRTVAAPSVFIFPPSDEQLKSGTASVVCLLNNFY |
| | | PREAKVQWKVDNALQSGNSQESVTEQDSKDSTYSL |
| | | SSTLTLSKADYEKHKVYACEVTHQGLSSPVTKSFNR |
| | | GEC |

TABLE D
Gantenerumab (HCDR1-3 and LCDR1-3 based on IMGT nomenclature)

| SEQ ID NO | Description | Amino Acid Sequence |
|---|---|---|
| SEQ ID NO: 31 | HCDR1 | GFTFSSYA |
| SEQ ID NO: 32 | HCDR2 | INASGTRT |
| SEQ ID NO: 33 | HCDR3 | ARGKGNTHKPYGYVRYFDV |
| SEQ ID NO: 34 | LCDR1 | QSVSSSY |
| SEQ ID NO: 35 | LCDR2 | GAS |
| SEQ ID NO: 36 | LCDR3 | LQIYNMPIT |
| SEQ ID NO: 37 | VH | QVELVESGGGLVQPGGSLRLSCAASGFTFSSYAMS |
| | | WVRQAPGKGLEWVSAINASGTRTYYADSVKGRFTI |
| | | SRDNSKNTLYLQMNSLRAEDTAV |
| SEQ ID NO: 38 | VL | IVLTQSPATLSLSPGERATLSCRASQSVSSSYLAWYQ |
| | | QKPGQAPRLLIYGASSRATGVPARFSGSGSGTDFTL |
| | | TISSLEPEDFATYYCLQIYNMP |
| SEQ ID NO: 39 | Heavy | QVELVESGGGLVQPGGSLRLSCAASGFTFSSYAMS |
| | chain | WVRQAPGKGLEWVSAINASGTRTYYADSVKGRFTI |
| | H-Gamma- | SRDNSKNTLYLQMNSLRAEDTAVYYCARGKGNTH |
| | 1 | KPYGYVRYFDVWGQGTLVTVSSASTKGPSVFPLAP |
| | | SSKSTSGGTAALGCLVKDYFPEPVTVSWNSGALTSG |
| | | VHTFPAVLQSSGLYSLSSVVTVPSSSLGTQTYICNVN |
| | | HKPSNTKVDKKVEPKSCDKTHTCPPCPAPELLGGPS |
| | | VFLFPPKPKDTLMISRTPEVTCVVVDVSHEDPEVKF |
| | | NWYVDGVEVHNAKTKPREEQYNSTYRVVSVLTVL |
| | | HQDWLNGKEYKCKVSNKALPAPIEKTISKAKGQPR |
| | | EPQVYTLPPSRDELTKNQVSLTCLVKGFYPSDIAVE |
| | | WESNGQPENNYKTTPPVLDSDGSFFLYSKLTVDKSR |
| | | WQQGNVFSCSVMHEALHNHYTQKSLSLSPGK |
| SEQ ID NO: 40 | Light chain | DIVLTQSPATLSLSPGERATLSCRASQSVSSSYLAWY |
| | L-Kappa | QQKPGQAPRLLIYGASSRATGVPARFSGSGSGTDFT |
| | | LTISSLEPEDFATYYCLQIYNMPITFGQGTKVEIKRT |
| | | VAAPSVFIFPPSDEQLKSGTASVVCLLNNFYPREAK |
| | | VQWKVDNALQSGNSQESVTEQDSKDSTYSLSSTLT |
| | | LSKADYEKHKVYACEVTHQGLSSPVTKSFNRGEC |

TABLE E
Ponezumab (HCDR1-3 and LCDR1-3 based on IMGT nomenclature)

| SEQ ID NO | Description | Amino Acid Sequence |
|---|---|---|
| SEQ ID NO: 41 | HCDR1 | GYYTEAYY |
| SEQ ID NO: 42 | HCDR2 | IDPATGNT |
| SEQ ID NO: 43 | HCDR3 | ASLYSLPVY |
| SEQ ID NO: 44 | LCDR1 | QSLLYSDAKTY |
| SEQ ID NO: 45 | LCDR2 | QIS |
| SEQ ID NO: 46 | LCDR3 | LQGTHYPVL |
| SEQ ID NO: 47 | VH | QVQLVQSGAEVKKPGASVKVSCKASGYYTEAYYIH |
| | | WVRQAPGQGLEWMGRIDPATGNTKYAPRLQDRVT |
| | | MTRDTSTSTVYMELSSLRSEDTAVYYCA |
| SEQ ID NO: 48 | VL | DVVMTQSPLSLPVTLGQPASISCKSSQSLLYSDAKT |
| | | YLNWFQQRPGQSPRRLIYQISRLDPGVPDRFSGSGS |
| | | GTDFTLKISRVEAEDVGVYYCLQGTHYP |
| SEQ ID NO: 49 | Heavy | QVQLVQSGAEVKKPGASVKVSCKASGYYTEAYYIH |
| | chain | WVRQAPGQGLEWMGRIDPATGNTKYAPRLQDRVT |
| | H-Gamma- | MTRDTSTSTVYMELSSLRSEDTAVYYCASLYSLPVY |
| | 2 | WGQGTTVTVSSASTKGPSVFPLAPCSRSTSESTAAL |
| | | GCLVKDYFPEPVTVSWNSGALTSGVHTFPAVLQSS |
| | | GLYSLSSVVTVPSSNFGTQTYTCNVDHKPSNTKVD |
| | | KTVERKCCVECPPCPAPPVAGPSVFLFPPKPKDTLMI |
| | | SRTPEVTCVVVDVSHEDPEVQFNWYVDGVEVHNA |
| | | KTKPREEQFNSTFRVVSVLTVVHQDWLNGKEYKCK |
| | | VSNKGLPSSIEKTISKTKGQPREPQVYTLPPSREEMT |
| | | KNQVSLTCLVKGFYPSDIAVEWESNGQPENNYKTT |
| | | PPMLDSDGSFFLYSKLTVDKSRWQQGNVFSCSVMH |
| | | EALHNHYTQKSLSLSPGK |
| SEQ ID NO: 50 | Light chain | DVVMTQSPLSLPVTLGQPASISCKSSQSLLYSDAKT |
| | L-Kappa | YLNWFQQRPGQSPRRLIYQISRLDPGVPDRFSGSGS |
| | | GTDFTLKISRVEAEDVGVYYCLQGTHYPVLFGQGT |
| | | RLEIKRTVAAPSVFIFPPSDEQLKSGTASVVCLLNNF |
| | | YPREAKVQWKVDNALQSGNSQESVTEQDSKDSTYS |
| | | LSSTLTLSKADYEKHKVYACEVTHQGLSSPVTKSFN |
| | | RGEC |

TABLE F
Bapineuzumab (HCDR1-3 and LCDR1-3 based on IMGT nomenclature)

| SEQ ID NO | Description | Amino Acid Sequence |
|---|---|---|
| SEQ ID NO: 51 | HCDR1 | GFTFSNYG |
| SEQ ID NO: 52 | HCDR2 | ASIRSGGGRT |
| SEQ ID NO: 53 | HCDR3 | VRYDHYSGSSDY |
| SEQ ID NO: 54 | LCDR1 | QSLLDSDGKTY |
| SEQ ID NO: 55 | LCDR2 | LVS |
| SEQ ID NO: 56 | LCDR3 | WQGTHFPRT |
| SEQ ID NO: 57 | VH | EVQLLESGGGLVQPGGSLRLSCAASGFTFSNYGMS |
| | | WVRQAPGKGLEWVASIRSGGGRTYYSDNVKGRFTI |
| | | SRDNSKNTLYLQMNSLRAEDTAVYYCVR |
| SEQ ID NO: 58 | VL | DVVMTQSPLSLPVTPGEPASISCKSSQSLLDSDGKTY |
| | | LNWLLQKPGQSPQRLIYLVSKLDSGVPDRFSGSGSG |
| | | TDFTLKISRVEAEDVGVYYCWQGTHFP |
| SEQ ID NO: 59 | Heavy | EVQLLESGGGLVQPGGSLRLSCAASGFTFSNYGMS |
| | chain | WVRQAPGKGLEWVASIRSGGGRTYYSDNVKGRFTI |
| | H-Gamma- | SRDNSKNTLYLQMNSLRAEDTAVYYCVRYDHYSG |
| | 1 | SSDYWGQGTLVTVSSASTKGPSVFPLAPSSKSTSGG |
| | | TAALGCLVKDYFPEPVTVSWNSGALTSGVHTFPAV |
| | | LQSSGLYSLSSVVTVPSSSLGTQTYICNVNHKPSNTK |
| | | VDKKVEPKSCKTHTCPPCPAPELLGGPSVFLFPPKPK |
| | | DTLMISRTPEVTCVVVDVSHEDPEVKFNWYVDGVE |
| | | VHNAKTKPREEQYNSTYRVVSVLTVLHQDWLNGK |
| | | EYKCKVSNKALPAPIEKTISKAKGQPREPQVYTLPPS |
| | | REEMTKNQVSLTCLVKGFYPSDIAVEWESNGQPEN |
| | | NYKTTPPVLDSDGSFFLYSKLTVDKSRWQQGNVFS |
| | | CSVMHEALHNKYTQKSLSLSPG |
| SEQ ID NO: 60 | Light chain | DVVMTQSPLSLPVTPGEPASISCKSSQSLLDSDGKTY |
| | L-Kappa | LNWLLQKPGQSPQRLIYLVSKLDSGVPDRFSGSGSG |
| | | TDFTLKISRVEAEDVGVYYCWQGTHFPRTFGQGTK |
| | | VEIKRTVAAPSVFIFPPSDEQLKSGTASVVCLLNNFY |
| | | PREAKVQWKVDNALQSGNSQESVTEQDSKDSTYSL |

TABLE G
Lecanemab (HCDR1-3 and LCDR1-3 based on IMGT nomenclature)

| SEQ ID NO | Description | Amino Acid Sequence |
|---|---|---|
| SEQ ID NO: 61 | HCDR1 | GFTFSSFG |
| SEQ ID NO: 62 | HCDR2 | ISSGSSTI |
| SEQ ID NO: 63 | HCDR3 | AREGGYYYGRSYYTMDY |
| SEQ ID NO: 64 | LCDR1 | QSIVHSNGNTY |
| SEQ ID NO: 65 | LCDR2 | KVS |
| SEQ ID NO: 66 | LCDR3 | FQGSHVPPT |
| SEQ ID NO: 67 | VH | EVQLVESGGGLVQPGGSLRLSCSASGFTFSSFGMH |
| | | WVRQAPGKGLEWVAYISSGSSTIYYGDTVKGRFTIS |
| | | RDNAKNSLFLQMSSLRAEDTAVYYCAR |
| SEQ ID NO: 68 | VL | DVVMTQSPLSLPVTPGAPASISCRSSQSIVHSNGNTY |
| | | LEWYLQKPGQSPKLLIYKVSNRFSGVPDRFSGSGSG |
| | | TDFTLRISRVEAEDVGIYYCFQGSHVP |
| SEQ ID NO: 69 | Heavy | EVQLVESGGGLVQPGGSLRLSCSASGFTFSSFGMH |
| | chain | WVRQAPGKGLEWVAYISSGSSTIYYGDTVKGRFTIS |
| | H-Gamma- | RDNAKNSLFLQMSSLRAEDTAVYYCAREGGYYYG |
| | 1 | RSYYTMDYWGQGTTVTVSSASTKGPSVFPLAPSSK |
| | | STSGGTAALGCLVKDYFPEPVTVSWNSGALTSGVH |
| | | TFPAVLQSSGLYSLSSVVTVPSSSLGTQTYICNVNHK |
| | | PSNTKVDKRVEPKSCDKTHTCPPCPAPELLGGPSVF |
| | | LFPPKPKDTLMISRTPEVTCVVVDVSHEDPEVKFNW |
| | | YVDGVEVHNAKTKPREEQYNSTYRVVSVLTVLHQ |
| | | DWLNGKEYKCKVSNKALPAPIEKTISKAKGQPREP |
| | | QVYTLPPSREEMTKNQVSLTCLVKGFYPSDIAVEWE |
| | | SNGQPENNYKTTPPVLDSDGSFFLYSKLTVDKSRW |
| | | QQGNVFSCSVMHEALHNHYTQKSLSLSPGK |
| SEQ ID NO: 70 | Light chain | DVVMTQSPLSLPVTPGAPASISCRSSQSIVHSNGNTY |
| | L-Kappa | LEWYLQKPGQSPKLLIYKVSNRFSGVPDRFSGSGSG |
| | | TDFTLRISRVEAEDVGIYYCFQGSHVPPTFGPGTKLE |
| | | IKRTVAAPSVFIFPPSDEQLKSGTASVVCLLNNFYPR |
| | | EAKVQWKVDNALQSGNSQESVTEQDSKDSTYSLSS |
| | | TLTLSKADYEKHKVYACEVTHQGLSSPVTKSFNRG |
| | | EC |

In some embodiments, the anti-amyloid beta antibody or antigen-binding fragment thereof comprises a human IgG1, IgG2, IgG3, or IgG4 constant domain. In some embodiments, the anti-amyloid beta antibody or antigen-binding fragment thereof comprises a human IgG1 constant region. In some embodiments, the anti-amyloid beta antibody or antigen-binding fragment thereof comprises a human IgG2 constant region. In some embodiments, the anti-amyloid beta antibody or antigen-binding fragment thereof comprises a human IgG3 constant region. In some embodiments, the anti-amyloid beta antibody or antigen-binding fragment thereof comprises a human IgG4 constant region.

In some embodiments, the anti-amyloid beta antibody or antigen-binding fragment thereof is a human anti-amyloid beta antibody or antigen-binding fragment thereof.

In some embodiments, the antigen-binding fragment is a single chain antibody, Fv, Fab, Fab′, F(ab′)₂, Fd, single chain Fv molecule (scFv), bispecific single chain Fv dimer, diabody, domain-deleted antibody or single domain antibody (dAb). In some embodiments, the anti-amyloid beta antibody is a whole antibody. In some embodiments, an anti-amyloid beta antibody is a single chain antibody. In some embodiments, an anti-amyloid beta antibody is a scFv. In some embodiments, an anti-amyloid beta antibody is a Fab. In some embodiments, an anti-amyloid beta antibody is a F(ab′)₂. In some embodiments, an anti-amyloid beta antibody is a Fv. In some embodiments, an anti-amyloid beta antibody is a Fd. In some embodiments, an anti-amyloid beta antibody is a bispecific single chain Fv dimer. In some embodiments, an anti-amyloid beta antibody is a diabody. In some embodiments, an anti-amyloid beta antibody is a single domain antibody (dAb). In some embodiments, an anti-amyloid beta antibody is a bispecific antibody.

In some embodiments, an anti-amyloid beta antibody, or an antigen-binding fragment thereof, of the disclosure is human. In some embodiments, the human anti-amyloid beta, or an antigen-binding fragment thereof, comprises a human IgH chain and a human Igκ chain. In some embodiments, the human anti-amyloid beta, or an antigen-binding fragment thereof, comprises a human IgH chain and a human Igλ chain. In some embodiments, the isotype of the anti-amyloid beta antibody is selected from IgM, IgD, IgG (such as IgG1, IgG2, IgG3, and IgG4), IgA and IgE. In some embodiments, the isotype of the anti-amyloid beta antibody is selected from IgG1, IgG2, IgG3, and IgG4.

In some embodiments, the anti-amyloid beta antibody binds an Fc receptor (FcR) selected from an FcγR, an FcεR, and an FcαR. In some embodiments, the anti-amyloid beta antibody binds an FcγR selected from FcγRI (CD64), FcγRII (CD32), and FcγRIII (CD16), including isoforms thereof. In some embodiments, the Fc region of the anti-amyloid beta antibody comprises a mutation so that it preferentially binds a particular FcγR.

In some embodiments, the anti-amyloid beta antibody or antigen binding fragment thereof binds to amyloid beta plaques, parenchymal amyloid, cerebrovascular amyloid, or diffuse amyloid beta deposits.

The pharmaceutical compositions provided herein can be administered by a variety of routes including oral, rectal, transdermal, subcutaneous, intravenous, intramuscular, and intranasal. Depending on the intended route of delivery, the compounds provided herein are preferably formulated as either injectable or oral compositions or as salves, as lotions or as patches all for transdermal administration.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable carriers known in the art. As before, the active compound in such compositions is typically a minor component, often being from about 0.05 to 10% by weight with the remainder being the injectable carrier and the like.

Transdermal compositions are typically formulated as a topical ointment or cream containing the active ingredient(s), generally in an amount ranging from about 0.01 to about 20% by weight, preferably from about 0.1 to about 20% by weight, preferably from about 0.1 to about 10% by weight, and more preferably from about 0.5 to about 15% by weight. When formulated as a ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or the formulation. All such known transdermal formulations and ingredients are included within the scope provided herein.

The compounds provided herein can also be administered by a transdermal device. Accordingly, transdermal administration can be accomplished using a patch either of the reservoir or porous membrane type, or of a solid matrix variety.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pennsylvania, which is incorporated herein by reference.

The above-described components for orally administrable, injectable, or topically administrable compositions are merely representative. Other materials as well as processing techniques and the like are set forth in Part 8 of Remington's The Science and Practice of Pharmacy, 21st edition, 2005, Publisher: Lippincott Williams & Wilkins, which is incorporated herein by reference.

The compounds of this disclosure can also be administered in sustained release forms or from sustained release drug delivery systems. A description of representative sustained release materials can be found in Remington's Pharmaceutical Sciences.

The present disclosure also relates to the pharmaceutically acceptable formulations of a compound described herein (e.g., a compound of Formulae (A-I), (B-I), (C-I), (D-I), (E-I), (F-I), (G-I), or (H-I), or a pharmaceutically acceptable salt thereof). In one embodiment, the formulation comprises water. In another embodiment, the formulation comprises a cyclodextrin derivative. The most common cyclodextrins are α-, β- and γ-cyclodextrins consisting of 6, 7 and 8 α-1,4-linked glucose units, respectively, optionally comprising one or more substituents on the linked sugar moieties, which include, but are not limited to, methylated, hydroxyalkylated, acylated, and sulfoalkylether substitution. In certain embodiments, the cyclodextrin is a sulfoalkyl ether β-cyclodextrin, e.g., for example, sulfobutyl ether β-cyclodextrin, also known as Captisol®. See, e.g., U.S. Pat. No. 5,376,645. In certain embodiments, the formulation comprises hexapropyl-β-cyclodextrin. In a more particular embodiment, the formulation comprises hexapropyl-β-cyclodextrin (10-50% in water).

The present disclosure also relates to the pharmaceutically acceptable acid addition salt of a compound described herein (e.g., a compound of Formulae (A-I), (B-I), (C-I), (D-I), (E-I), (F-I), (G-I), or (H-I)). The acid which may be used to prepare the pharmaceutically acceptable salt is that which forms a non-toxic acid addition salt, i.e., a salt containing pharmacologically acceptable anions such as the hydrochloride, hydroiodide, hydrobromide, nitrate, sulfate, bisulfate, phosphate, acetate, lactate, citrate, tartrate, succinate, maleate, fumarate, benzoate, para-toluenesulfonate, and the like.

The following formulation examples illustrate representative pharmaceutical compositions that may be prepared in accordance with this disclosure. The present disclosure, however, is not limited to the following pharmaceutical compositions.

Exemplary Formulation 1-Tablets: A compound described herein (e.g., a compound of Formulae (A-I), (B-I), (C-I), (D-I), (E-I), (F-I), (G-I), or (H-I), or a pharmaceutically acceptable salt thereof), may be admixed as a dry powder with a dry gelatin binder in an approximate 1:2 weight ratio. A minor amount of magnesium stearate is added as a lubricant. The mixture is formed into 240-270 mg tablets (80-90 mg of active compound per tablet) in a tablet press.

Exemplary Formulation 2-Capsules: A compound described herein (e.g., a compound of Formulae (A-I), (B-I), (C-I), (D-I), (E-I), (F-I), (G-I), or (H-I), or a pharmaceutically acceptable salt thereof), may be admixed as a dry powder with a starch diluent in an approximate 1:1 weight ratio. The mixture is filled into 250 mg capsules (125 mg of active compound per capsule).

Exemplary Formulation 3-Liquid: A compound described herein (e.g., a compound of Formulae (A-I), (B-I), (C-I), (D-I), (E-I), (F-I), (G-I), or (H-I), or a pharmaceutically acceptable salt thereof) (125 mg) may be admixed with sucrose (1.75 g) and xanthan gum (4 mg) and the resultant mixture may be blended, passed through a No. 10 mesh U.S. sieve, and then mixed with a previously made solution of microcrystalline cellulose and sodium carboxymethyl cellulose (11:89, 50 mg) in water. Sodium benzoate (10 mg), flavor, and color are diluted with water and added with stirring. Sufficient water may then be added to produce a total volume of 5 mL.

Exemplary Formulation 4-Tablets: A compound described herein (e.g., a compound of Formulae (A-I), (B-I), (C-I), (D-I), (E-I), (F-I), (G-I), or (H-I), or a pharmaceutically acceptable salt thereof), may be admixed as a dry powder with a dry gelatin binder in an approximate 1:2 weight ratio. A minor amount of magnesium stearate is added as a lubricant. The mixture is formed into 450-900 mg tablets (150-300 mg of active compound) in a tablet press.

Exemplary Formulation 5-Injection: A compound described herein (e.g., a compound of Formulae (A-I), (B-I), (C-I), (D-I), (E-I), (F-I), (G-I), or (H-I), or a pharmaceutically acceptable salt thereof), may be dissolved or suspended in a buffered sterile saline injectable aqueous medium to a concentration of approximately 5 mg/mL.

Exemplary Formulation 6-Tablets: A compound described herein (e.g., a compound of Formulae (A-I), (B-I), (C-I), (D-I), (E-I), (F-I), (G-I), or (H-I), or a pharmaceutically acceptable salt thereof), may be admixed as a dry powder with a dry gelatin binder in an approximate 1:2 weight ratio. A minor amount of magnesium stearate is added as a lubricant. The mixture is formed into 90-150 mg tablets (30-50 mg of active compound per tablet) in a tablet press.

Exemplary Formulation 7-Tablets: A compound described herein (e.g., a compound of Formulae (A-I), (B-I), (C-I), (D-I), (E-I), (F-I), (G-I), or (H-I), or a pharmaceutically acceptable salt thereof), may be admixed as a dry powder with a dry gelatin binder in an approximate 1:2 weight ratio. A minor amount of magnesium stearate is added as a lubricant. The mixture is formed into 30-90 mg tablets (10-30 mg of active compound per tablet) in a tablet press.

Exemplary Formulation 8-Tablets: A compound described herein (e.g., a compound of Formulae (A-I), (B-I), (C-I), (D-I), (E-I), (F-I), (G-I), or (H-I), or a pharmaceutically acceptable salt thereof), may be admixed as a dry powder with a dry gelatin binder in an approximate 1:2 weight ratio. A minor amount of magnesium stearate is added as a lubricant. The mixture is formed into 0.3-30 mg tablets (0.1-10 mg of active compound per tablet) in a tablet press.

Exemplary Formulation 9-Tablets: A compound described herein (e.g., a compound of Formulae (A-I), (B-I), (C-I), (D-I), (E-I), (F-I), (G-I), or (H-I), or a pharmaceutically acceptable salt thereof), may be admixed as a dry powder with a dry gelatin binder in an approximate 1:2 weight ratio. A minor amount of magnesium stearate is added as a lubricant. The mixture is formed into 150-240 mg tablets (50-80 mg of active compound per tablet) in a tablet press.

Exemplary Formulation 10-Tablets: A compound described herein (e.g., a compound of Formulae (A-I), (B-I), (C-I), (D-I), (E-I), (F-I), (G-I), or (H-I), or a pharmaceutically acceptable salt thereof), may be admixed as a dry powder with a dry gelatin binder in an approximate 1:2 weight ratio. A minor amount of magnesium stearate is added as a lubricant. The mixture is formed into 270-450 mg tablets (90-150 mg of active compound per tablet) in a tablet press.

Injection dose levels range from about 0.1 mg/kg/hour to at least 10 mg/kg/hour, all for from about 1 to about 120 hours and especially 24 to 96 hours. A preloading bolus of from about 0.1 mg/kg to about 10 mg/kg or more may also be administered to achieve adequate steady state levels. The maximum total dose is not expected to exceed about 2 g/day for a 40 to 80 kg human patient.

For the prevention and/or treatment of long-term conditions the regimen for treatment usually stretches over many months or years so oral dosing is preferred for patient convenience and tolerance. With oral dosing, one to five and especially two to four and typically three oral doses per day are representative regimens. Using these dosing patterns, each dose provides from about 0.01 to about 20 mg/kg of the compound provided herein, with preferred doses each providing from about 0.1 to about 10 mg/kg, and especially about 1 to about 5 mg/kg.

Transdermal doses are generally selected to provide similar or lower blood levels than are achieved using injection doses.

When used to prevent the onset of a CNS-disorder, the compounds provided herein will be administered to a subject at risk for developing the condition, typically on the advice and under the supervision of a physician, at the dosage levels described above. Subjects at risk for developing a particular condition generally include those that have a family history of the condition, or those who have been identified by genetic testing or screening to be particularly susceptible to developing the condition.

### Methods of Treatment and Use

Compounds of the present disclosure (e.g., a compound of Formulae (A-I), (B-I), (C-I), (D-I), (E-I), (F-I), (G-I), or (H-I), or a pharmaceutically acceptable salt thereof), as described herein, are generally designed to be positive allosteric modulators of NMDA receptor function, and therefore are useful for the treatment and prevention of, e.g., CNS-related conditions in a subject.

In some embodiments, the compounds described herein (e.g., a compound of Formulae (A-I), (B-I), (C-I), (D-I), (E-I), (F-I), (G-I), or (H-I), or a pharmaceutically acceptable salt thereof), as described herein, are generally designed to penetrate the blood brain barrier (e.g., designed to be transported across the blood brain barrier), and act as positive allosteric modulators (PAM) of NMDA receptors, and potentiate NMDA receptor function.

In one aspect, the disclosure provides a method for treating a disease, disorder or condition requiring positive allosteric NMDA modulation in a subject, comprising administering to the subject an effective amount of a compound or pharmaceutically acceptable salt thereof, or a pharmaceutical composition as disclosed herein.

In one aspect, the disclosure provides a method for treating a CNS-related condition in a subject, comprising administering to the subject an effective amount of a compound or pharmaceutically acceptable salt thereof, or a pharmaceutical composition as disclosed herein.

In one aspect, the disclosure provides a method for preventing a disease, disorder or condition requiring positive allosteric NMDA modulation in a subject, comprising administering to the subject an effective amount of a compound or pharmaceutically acceptable salt thereof, or a pharmaceutical composition as disclosed herein.

In one aspect, the disclosure provides a method for preventing a CNS-related condition in a subject, comprising administering to the subject an effective amount of a compound or pharmaceutically acceptable salt thereof, or a pharmaceutical composition as disclosed herein.

In one aspect, the disclosure provides a method for inducing sedation or anesthesia in a subject, comprising administering to the subject an effective amount of a compound or pharmaceutically acceptable salt thereof, or a pharmaceutical composition as disclosed herein.

In one aspect, the disclosure provides a compound or pharmaceutically acceptable salt thereof, or pharmaceutical composition as disclosed herein for use in treating a disease, disorder or condition requiring positive allosteric NMDA modulation in a subject.

In one aspect, the disclosure provides a compound or pharmaceutically acceptable salt thereof, or pharmaceutical composition as disclosed herein for use in treating a CNS-related condition in a subject.

In one aspect, the disclosure provides a compound or pharmaceutically acceptable salt thereof, or pharmaceutical composition as disclosed herein for use in preventing a disease, disorder or condition requiring positive allosteric NMDA modulation in a subject.

In one aspect, the disclosure provides a compound or pharmaceutically acceptable salt thereof, or pharmaceutical composition as disclosed herein for use in preventing a CNS-related condition in a subject.

In one aspect, the disclosure provides a compound or pharmaceutically acceptable salt thereof, or pharmaceutical composition as disclosed herein for use in inducing sedation or anesthesia in a subject.

In one aspect, the disclosure provides the use of a compound or pharmaceutically acceptable salt thereof, or pharmaceutical composition as disclosed herein for the manufacture of a medicament for treating a disease, disorder or condition requiring positive allosteric NMDA modulation in a subject.

In one aspect, the disclosure provides the use compound or pharmaceutically acceptable salt thereof, or pharmaceutical composition as disclosed herein for the manufacture of a medicament for treating a CNS-related condition in a subject.

In one aspect, the disclosure provides the use of a compound or pharmaceutically acceptable salt thereof, or pharmaceutical composition as disclosed herein for the manufacture of a medicament for preventing a disease, disorder or condition requiring positive allosteric NMDA modulation in a subject.

In one aspect, the disclosure provides the use compound or pharmaceutically acceptable salt thereof, or pharmaceutical composition as disclosed herein for the manufacture of a medicament for preventing a CNS-related condition in a subject.

In one aspect, the disclosure provides the use of compound or pharmaceutically acceptable salt thereof, or pharmaceutical composition as disclosed herein for the manufacture of a medicament for inducing sedation or anesthesia in a subject.

In some embodiments, the method of treating a disease, disorder or condition comprises administering a compound of the disclosure in combination with a) an anti-amyloid beta antibody or antigen-binding fragment thereof. In some embodiments, the method of preventing a disease, disorder or condition comprises administering a compound of the disclosure in combination with a) an anti-amyloid beta antibody or antigen-binding fragment thereof.

In some embodiments, the disorder is cancer. In some embodiments, the disorder is diabetes. In some embodiments, the disorder is a sterol synthesis disorder. In some embodiments, the disorder is a gastrointestinal (GI) disorder, e.g., constipation, irritable bowel syndrome (IBS), inflammatory bowel disease (IBD) (e.g., ulcerative colitis, Crohn's disease), structural disorders affecting the GI, anal disorders (e.g., hemorrhoids, internal hemorrhoids, external hemorrhoids, anal fissures, perianal abscesses, anal fistula), colon polyps, cancer, colitis. In some embodiments, the disorder is inflammatory bowel disease.

In some embodiments, the disorder is Smith-Lemli-Opitz Syndrome (SLOS). In some embodiments, the disorder is desmosterolosis. In some embodiments, the disorder is sitosterolemia. In some embodiments, the disorder is cerebrotendinous xanthomatosis (CTX). In some embodiments, the disorder is Mevalonate Kinase Deficiency (MKD). In some embodiments, the disorder is SC4MOL gene mutation (SMO Deficiency). In some embodiments, the disorder is Niemann-Pick disease. In some embodiments, the disorder is autism spectrum disorder (ASD). In some embodiments, the disorder is associated with phenylketonuria.

Exemplary conditions related to positive NMDA-modulation includes, but are not limited to, gastrointestinal (GI) disorder, e.g., constipation, irritable bowel syndrome (IBS), inflammatory bowel disease (IBD) (e.g., ulcerative colitis, Crohn's disease), structural disorders affecting the GI, anal disorders (e.g., hemorrhoids, internal hemorrhoids, external hemorrhoids, anal fissures, perianal abscesses, anal fistula), colon polyps, cancer, colitis, and CNS conditions, e.g., as described herein.

Exemplary CNS conditions related to positive NMDA-modulation include, but are not limited to, adjustment disorders, anxiety disorders (including obsessive-compulsive disorder, posttraumatic stress disorder, social phobia, generalized anxiety disorder), cognitive disorders (including Alzheimer's disease and other forms of dementia (e.g., frontotemporal dementia)), dissociative disorders, eating disorders, mood disorders (including depression (e.g., postpartum depression), bipolar disorder, dysthymic disorder, suicidality), schizophrenia or other psychotic disorders (including schizoaffective disorder), sleep disorders (including insomnia), substance abuse-related disorders, personality disorders (including obsessive-compulsive personality disorder), autism spectrum disorders (including those involving mutations to the Shank group of proteins (e.g., Shank3)), neurodevelopmental disorders (including Rett syndrome), multiple sclerosis, sterol synthesis disorders, pain (including acute and chronic pain; headaches, e.g., migraine headaches), seizure disorders (including status epilepticus and monogenic forms of epilepsy such as Dravet's disease, and Tuberous Sclerosis Complex (TSC)), stroke, traumatic brain injury, movement disorders (including Huntington's disease and Parkinson's disease) and tinnitus.

In certain embodiments, the compound of the present disclosure, e.g., a compound of Formula (I), and pharmaceutically acceptable salts thereof, can be used to induce sedation or anesthesia. In certain embodiments, the compound described herein (e.g., a compound of Formulae (A-I), (B-I), (C-I), (D-I), (E-I), (F-I), (G-I), or (H-I), or a pharmaceutically acceptable salt thereof), is useful in the treatment or prevention of adjustment disorders, anxiety disorders, cognitive disorders, dissociative disorders, eating disorders, mood disorders, schizophrenia or other psychotic disorders, sleep disorders, substance-related disorders, personality disorders, autism spectrum disorders, neurodevelopmental disorders, sterol synthesis disorders, pain, seizure disorders, stroke, traumatic brain injury, movement disorders and vision impairment, hearing loss, and tinnitus. In some embodiments, the disorder is Huntington's disease. In some embodiments, the disorder is Parkinson's disease. In some embodiments, the disorder is an inflammatory disease (e.g., lupus).

In another aspect, provided is a method of treating or preventing brain excitability in a subject susceptible to or afflicted with a condition associated with brain excitability, comprising administering to the subject an effective amount of a compound of the present disclosure (e.g., a compound of Formulae (A-I), (B-I), (C-I), (D-I), (E-I), (F-I), (G-I), or (H-I), or a pharmaceutically acceptable salt thereof).

In yet another aspect, the present disclosure provides a combination of a compound of the present disclosure (e.g., a compound of Formulae (A-I), (B-I), (C-I), (D-I), (E-I), (F-I), (G-I), or (H-I), or a pharmaceutically acceptable salt thereof), and another pharmacologically active agent. The compounds provided herein can be administered as the sole active agent or they can be administered in combination with other agents. Administration in combination can proceed by any technique apparent to those of skill in the art including, for example, separate, sequential, concurrent and alternating administration.

### Diseases and Disorders

### Movement Disorders

Also described herein are methods for treating a movement disorder. As used herein, “movement disorders” refers to a variety of diseases and disorders that are associated with hyperkinetic movement disorders and related abnormalities in muscle control. Exemplary movement disorders include, but are not limited to, Parkinson's disease and Parkinsonism (defined particularly by bradykinesia), dystonia, chorea and Huntington's disease, ataxia, tremor (e.g., essential tremor), myoclonus and startle, tics and Tourette syndrome, Restless legs syndrome, stiff person syndrome, and gait disorders.

Tremor is an involuntary, at times rhythmic, muscle contraction and relaxation that can involve oscillations or twitching of one or more body parts (e.g., hands, arms, eyes, face, head, vocal folds, trunk, legs). Tremor includes hereditary, degenerative, and idiopathic disorders such as Wilson's disease, Parkinson's disease, and essential tremor, respectively; metabolic diseases (e.g., thyroid-parathyroid-, liver disease and hypoglycemia); peripheral neuropathies (associated with Charcot-Marie-Tooth, Roussy-Levy, diabetes mellitus, complex regional pain syndrome); toxins (nicotine, mercury, lead, CO, Manganese, arsenic, toluene); drug-induced (narcoleptics, tricyclics, lithium, cocaine, alcohol, adrenaline, bronchodilators, theophylline, caffeine, steroids, valproate, amiodarone, thyroid hormones, vincristine); and psychogenic disorders. Clinical tremor can be classified into physiologic tremor, enhanced physiologic tremor, essential tremor syndromes (including classical essential tremor, primary orthostatic tremor, and task- and position-specific tremor), dystonic tremor, parkinsonian tremor, cerebellar tremor, Holmes' tremor (i.e., rubral tremor), palatal tremor, neuropathic tremor, toxic or drug-induced tremor, and psychogenic tremor. Other forms of tremor include cerebellar tremor or intention tremor, dystonic tremor, essential tremor, orthostatic tremor, parkinsonian tremor, physiological tremor, psychogenic tremor, or rubral tremor.

Cerebellar tremor or intention tremor is a slow, broad tremor of the extremities that occurs after a purposeful movement. Cerebellar tremor is caused by lesions in or damage to the cerebellum resulting from, e.g., tumor, stroke, disease (e.g., multiple sclerosis, an inherited degenerative disorder).

Dystonic tremor occurs in individuals affected by dystonia, a movement disorder in which sustained involuntary muscle contractions cause twisting and repetitive motions and/or painful and abnormal postures or positions. Dystonic tremor may affect any muscle in the body. Dystonic tremors occurs irregularly and often can be relieved by complete rest.

Essential tremor or benign essential tremor is the most common type of tremor. Essential tremor may be mild and nonprogressive in some, and may be slowly progressive, starting on one side of the body but affect both sides within 3 years. The hands are most often affected, but the head, voice, tongue, legs, and trunk may also be involved. Tremor frequency may decrease as the person ages, but severity may increase. Heightened emotion, stress, fever, physical exhaustion, or low blood sugar may trigger tremors and/or increase their severity. Symptoms generally evolve over time and can be both visible and persistent following onset.

Orthostatic tremor is characterized by fast (e.g., greater than 12 Hz) rhythmic muscle contractions that occurs in the legs and trunk immediately after standing. Cramps are felt in the thighs and legs and the patient may shake uncontrollably when asked to stand in one spot. Orthostatic tremor may occur in patients with essential tremor.

Parkinsonian tremor is caused by damage to structures within the brain that control movement. Parkinsonian tremor is often a precursor to Parkinson's disease and is typically seen as a “pill-rolling” action of the hands that may also affect the chin, lips, legs, and trunk. Onset of parkinsonian tremor typically begins after age 60. Movement starts in one limb or on one side of the body and can progress to include the other side.

Physiological tremor can occur in normal individuals and have no clinical significance. It can be seen in all voluntary muscle groups. Physiological tremor can be caused by certain drugs, alcohol withdrawal, or medical conditions including an overactive thyroid and hypoglycemia. The tremor classically has a frequency of about 10 Hz.

Psychogenic tremor or hysterical tremor can occur at rest or during postural or kinetic movement. Patient with psychogenic tremor may have a conversion disorder or another psychiatric disease.

Rubral tremor is characterized by coarse slow tremor which can be present at rest, at posture, and with intention. The tremor is associated with conditions that affect the red nucleus in the midbrain, classical unusual strokes.

Parkinson's disease affects nerve cells in the brain that produce dopamine. Symptoms include muscle rigidity, tremors, and changes in speech and gait. Parkinsonism is characterized by tremor, bradykinesia, rigidity, and postural instability. Parkinsonism shares symptoms found in Parkinson's disease, but is a symptom complex rather than a progressive neurodegenerative disease.

Dystonia is a movement disorder characterized by sustained or intermittent muscle contractions causing abnormal, often repetitive movements or postures. Dystonic movements can be patterned, twisting, and may be tremulous. Dystonia is often initiated or worsened by voluntary action and associated with overflow muscle activation.

Chorea is a neurological disorder characterized by jerky involuntary movements typically affecting the shoulders, hips, and face.

Huntington's Disease is an inherited disease that causes nerve cells in the brain to waste away. Symptoms include uncontrolled movements, clumsiness, and balance problems. Huntington's disease can hinder walk, talk, and swallowing.

Ataxia refers to the loss of full control of bodily movements, and may affect the fingers, hands, arms, legs, body, speech, and eye movements.

Myoclonus and Startle is a response to a sudden and unexpected stimulus, which can be acoustic, tactile, visual, or vestibular.

Tics are an involuntary movement usually onset suddenly, brief, repetitive, but non-rhythmical, typically imitating normal behavior and often occurring out of a background of normal activity. Tics can be classified as motor or vocal, motor tics associated with movements while vocal tics associated with sound. Tics can be characterized as simple or complex. For example, simple motor tics involve only a few muscles restricted to a specific body part.

Tourette Syndrome is an inherited neuropsychiatric disorder with onset in childhood, characterized by multiple motor tics and at least one vocal tic.

Restless Legs Syndrome is a neurologic sensorimotor disorder characterized by an overwhelming urge to move the legs when at rest.

Stiff Person Syndrome is a progressive movement disorder characterized by involuntary painful spasms and rigidity of muscles, usually involving the lower back and legs. Stiff-legged gait with exaggerated lumbar hyperlordosis typically results. Characteristic abnormality on EMG recordings with continuous motor unit activity of the paraspinal axial muscles is typically observed. Variants include “stiff-limb syndrome” producing focal stiffness typically affecting distal legs and feet.

Gait disorders refer to an abnormality in the manner or style of walking, which results from neuromuscular, arthritic, or other body changes. Gait is classified according to the system responsible for abnormal locomotion, and include hemiplegic gait, diplegic gait, neuropathic gait, myopathic gait, parkinsonian gait, choreiform gait, ataxic gait, and sensory gait.

### Mood Disorders

Also provided herein are methods for treating a mood disorder, for example clinical depression, postnatal depression or postpartum depression, perinatal depression, atypical depression, melancholic depression, psychotic major depression, cationic depression, seasonal affective disorder, dysthymia, double depression, depressive personality disorder, recurrent brief depression, minor depressive disorder, bipolar disorder or manic depressive disorder, depression caused by chronic medical conditions, treatment-resistant depression, refractory depression, suicidality, suicidal ideation, or suicidal behavior.

Clinical depression is also known as major depression, major depressive disorder (MDD), severe depression, unipolar depression, unipolar disorder, and recurrent depression, and refers to a mental disorder characterized by pervasive and persistent low mood that is accompanied by low self-esteem and loss of interest or pleasure in normally enjoyable activities. Some people with clinical depression have trouble sleeping, lose weight, and generally feel agitated and irritable. Clinical depression affects how an individual feels, thinks, and behaves and may lead to a variety of emotional and physical problems. Individuals with clinical depression may have trouble doing day-to-day activities and make an individual feel as if life is not worth living.

Postnatal depression (PND) is also referred to as postpartum depression (PPD), and refers to a type of clinical depression that affects women after childbirth. Symptoms can include sadness, fatigue, changes in sleeping and eating habits, reduced sexual desire, crying episodes, anxiety, and irritability. In some embodiments, the PND is a treatment-resistant depression (e.g., a treatment-resistant depression as described herein). In some embodiments, the PND is refractory depression (e.g., a refractory depression as described herein).

In some embodiments, a subject having PND also experienced depression, or a symptom of depression during pregnancy. This depression is referred to herein as perinatal depression. In an embodiment, a subject experiencing perinatal depression is at increased risk of experiencing PND.

Atypical depression (AD) is characterized by mood reactivity (e.g., paradoxical anhedonia) and positivity, significant weight gain or increased appetite. Patients suffering from AD also may have excessive sleep or somnolence (hypersomnia), a sensation of limb heaviness, and significant social impairment as a consequence of hypersensitivity to perceived interpersonal rejection.

Melancholic depression is characterized by loss of pleasure (anhedonia) in most or all activities, failures to react to pleasurable stimuli, depressed mood more pronounced than that of grief or loss, excessive weight loss, or excessive guilt.

Psychotic major depression (PMD) or psychotic depression refers to a major depressive episode, in particular of melancholic nature, where the individual experiences psychotic symptoms such as delusions and hallucinations.

Catatonic depression refers to major depression involving disturbances of motor behavior and other symptoms. An individual may become mute and stuporose, and either is immobile or exhibits purposeless or bizarre movements.

Seasonal affective disorder (SAD) refers to a type of seasonal depression wherein an individual has seasonal patterns of depressive episodes coming on in the fall or winter.

Dysthymia refers to a condition related to unipolar depression, where the same physical and cognitive problems are evident. They are not as severe and tend to last longer (e.g., at least 2 years).

Double depression refers to fairly depressed mood (dysthymia) that lasts for at least 2 years and is punctuated by periods of major depression.

Depressive Personality Disorder (DPD) refers to a personality disorder with depressive features.

Recurrent Brief Depression (RBD) refers to a condition in which individuals have depressive episodes about once per month, each episode lasting 2 weeks or less and typically less than 2-3 days.

Minor depressive disorder or minor depression refers to a depression in which at least 2 symptoms are present for 2 weeks.

Bipolar disorder or manic depressive disorder causes extreme mood swings that include emotional highs (mania or hypomania) and lows (depression). During periods of mania the individual may feel or act abnormally happy, energetic, or irritable. They often make poorly thought out decisions with little regard to the consequences. The need for sleep is usually reduced. During periods of depression there may be crying, poor eye contact with others, and a negative outlook on life. The risk of suicide among those with the disorder is high at greater than 6% over 20 years, while self-harm occurs in 30-40%. Other mental health issues such as anxiety disorder and substance use disorder are commonly associated with bipolar disorder.

Depression caused by chronic medical conditions refers to depression caused by chronic medical conditions such as cancer or chronic pain, chemotherapy, chronic stress.

Treatment-resistant depression refers to a condition where the individuals have been treated for depression, but the symptoms do not improve. For example, antidepressants or psychological counseling (psychotherapy) do not ease depression symptoms for individuals with treatment-resistant depression. In some cases, individuals with treatment-resistant depression improve symptoms, but come back. Refractory depression occurs in patients suffering from depression who are resistant to standard pharmacological treatments, including tricyclic antidepressants, MAOIs, SSRIs, and double and triple uptake inhibitors and/or anxiolytic drugs, as well as non-pharmacological treatments (e.g., psychotherapy, electroconvulsive therapy, vagus nerve stimulation and/or transcranial magnetic stimulation).

Suicidality, suicidal ideation, suicidal behavior refers to the tendency of an individual to commit suicide. Suicidal ideation concerns thoughts about or an unusual preoccupation with suicide. The range of suicidal ideation varies greatly, from e.g., fleeting thoughts to extensive thoughts, detailed planning, role playing, incomplete attempts. Symptoms include talking about suicide, getting the means to commit suicide, withdrawing from social contact, being preoccupied with death, feeling trapped or hopeless about a situation, increasing use of alcohol or drugs, doing risky or self-destructive things, saying goodbye to people as if they won't be seen again.

Symptoms of depression include persistent anxious or sad feelings, feelings of helplessness, hopelessness, pessimism, worthlessness, low energy, restlessness, difficulty sleeping, sleeplessness, irritability, fatigue, motor challenges, loss of interest in pleasurable activities or hobbies, loss of concentration, loss of energy, poor self-esteem, absence of positive thoughts or plans, excessive sleeping, overeating, appetite loss, insomnia, self-harm, thoughts of suicide, and suicide attempts. The presence, severity, frequency, and duration of symptoms may vary on a case to case basis. Symptoms of depression, and relief of the same, may be ascertained by a physician or psychologist (e.g., by a mental state examination).

### Anxiety Disorders

Provided herein are methods for treating anxiety disorders. Anxiety disorder is a blanket term covering several different forms of abnormal and pathological fear and anxiety. Current psychiatric diagnostic criteria recognize a wide variety of anxiety disorders.

Generalized anxiety disorder is a common chronic disorder characterized by long-lasting anxiety that is not focused on any one object or situation. Those suffering from generalized anxiety experience non-specific persistent fear and worry and become overly concerned with everyday matters. Generalized anxiety disorder is the most common anxiety disorder to affect older adults.

In panic disorder, a person suffers from brief attacks of intense terror and apprehension, often marked by trembling, shaking, confusion, dizziness, nausea, difficulty breathing. These panic attacks, defined by the APA as fear or discomfort that abruptly arises and peaks in less than ten minutes, can last for several hours and can be triggered by stress, fear, or even exercise; although the specific cause is not always apparent. In addition to recurrent unexpected panic attacks, a diagnosis of panic disorder also requires that said attacks have chronic consequences: either worry over the attacks' potential implications, persistent fear of future attacks, or significant changes in behavior related to the attacks. Accordingly, those suffering from panic disorder experience symptoms even outside of specific panic episodes. Often, normal changes in heartbeat are noticed by a panic sufferer, leading them to think something is wrong with their heart or they are about to have another panic attack. In some cases, a heightened awareness (hypervigilance) of body functioning occurs during panic attacks, wherein any perceived physiological change is interpreted as a possible life-threatening illness (i.e. extreme hypochondriasis).

Obsessive compulsive disorder is a type of anxiety disorder primarily characterized by repetitive obsessions (distressing, persistent, and intrusive thoughts or images) and compulsions (urges to perform specific acts or rituals). The OCD thought pattern may be likened to superstitions insofar as it involves a belief in a causative relationship where, in reality, one does not exist. Often the process is entirely illogical; for example, the compulsion of walking in a certain pattern may be employed to alleviate the obsession of impending harm. And in many cases, the compulsion is entirely inexplicable, simply an urge to complete a ritual triggered by nervousness. In a minority of cases, sufferers of OCD may only experience obsessions, with no overt compulsions; a much smaller number of sufferers experience only compulsions.

The single largest category of anxiety disorders is that of phobia, which includes all cases in which fear and anxiety is triggered by a specific stimulus or situation. Sufferers typically anticipate terrifying consequences from encountering the object of their fear, which can be anything from an animal to a location to a bodily fluid.

Post-traumatic stress disorder or PTSD is an anxiety disorder which results from a traumatic experience. Post-traumatic stress can result from an extreme situation, such as combat, rape, hostage situations, or even serious accident. It can also result from long term (chronic) exposure to a severe stressor, for example soldiers who endure individual battles but cannot cope with continuous combat. Common symptoms include flashbacks, avoidant behaviors, and depression.

### Epilepsy

Epilepsy is a brain disorder characterized by repeated seizures over time. Types of epilepsy can include, but are not limited to generalized epilepsy, e.g., childhood absence epilepsy, juvenile myoclonic epilepsy, epilepsy with grand-mal seizures on awakening, West syndrome, Lennox-Gastaut syndrome, partial epilepsy, e.g., temporal lobe epilepsy, frontal lobe epilepsy, benign focal epilepsy of childhood.

### Epileptogenesis

Epileptogenesis is a gradual process by which a normal brain develops epilepsy (a chronic condition in which seizures occur). Epileptogenesis results from neuronal damage precipitated by the initial insult (e.g., status epilepticus).

### Status Epilepticus (SE)

Status epilepticus (SE) can include, e.g., convulsive status epilepticus, e.g., early status epilepticus, established status epilepticus, refractory status epilepticus, super-refractory status epilepticus; non-convulsive status epilepticus, e.g., generalized status epilepticus, complex partial status epilepticus; generalized periodic epileptiform discharges; and periodic lateralized epileptiform discharges. Convulsive status epilepticus is characterized by the presence of convulsive status epileptic seizures, and can include early status epilepticus, established status epilepticus, refractory status epilepticus, super-refractory status epilepticus. Early status epilepticus is treated with a first line therapy. Established status epilepticus is characterized by status epileptic seizures which persist despite treatment with a first line therapy, and a second line therapy is administered. Refractory status epilepticus is characterized by status epileptic seizures which persist despite treatment with a first line and a second line therapy, and a general anesthetic is generally administered. Super refractory status epilepticus is characterized by status epileptic seizures which persist despite treatment with a first line therapy, a second line therapy, and a general anesthetic for 24 hours or more.

Non-convulsive status epilepticus can include, e.g., focal non-convulsive status epilepticus, e.g., complex partial non-convulsive status epilepticus, simple partial non-convulsive status epilepticus, subtle non-convulsive status epilepticus; generalized non-convulsive status epilepticus, e.g., late onset absence non-convulsive status epilepticus, atypical absence non-convulsive status epilepticus, or typical absence non-convulsive status epilepticus.

### Seizure

A seizure is the physical findings or changes in behavior that occur after an episode of abnormal electrical activity in the brain. The term “seizure” is often used interchangeably with “convulsion.” Convulsions are when a person's body shakes rapidly and uncontrollably. During convulsions, the person's muscles contract and relax repeatedly.

Based on the type of behavior and brain activity, seizures are divided into two broad categories: generalized and partial (also called local or focal). Classifying the type of seizure helps doctors diagnose whether or not a patient has epilepsy.

Generalized seizures are produced by electrical impulses from throughout the entire brain, whereas partial seizures are produced (at least initially) by electrical impulses in a relatively small part of the brain. The part of the brain generating the seizures is sometimes called the focus.

There are six types of generalized seizures. The most common and dramatic, and therefore the most well-known, is the generalized convulsion, also called the grand-mal seizure. In this type of seizure, the patient loses consciousness and usually collapses. The loss of consciousness is followed by generalized body stiffening (called the “tonic” phase of the seizure) for 30 to 60 seconds, then by violent jerking (the “clonic” phase) for 30 to 60 seconds, after which the patient goes into a deep sleep (the “postictal” or after-seizure phase). During grand-mal seizures, injuries and accidents may occur, such as tongue biting and urinary incontinence.

Absence seizures cause a short loss of consciousness (just a few seconds) with few or no symptoms. The patient, most often a child, typically interrupts an activity and stares blankly. These seizures begin and end abruptly and may occur several times a day. Patients are usually not aware that they are having a seizure, except that they may be aware of “losing time.”

Myoclonic seizures consist of sporadic jerks, usually on both sides of the body. Patients sometimes describe the jerks as brief electrical shocks. When violent, these seizures may result in dropping or involuntarily throwing objects.

Clonic seizures are repetitive, rhythmic jerks that involve both sides of the body at the same time.

Tonic seizures are characterized by stiffening of the muscles.

Atonic seizures consist of a sudden and general loss of muscle tone, particularly in the arms and legs, which often results in a fall.

Seizures described herein can include epileptic seizures; acute repetitive seizures; cluster seizures; continuous seizures; unremitting seizures; prolonged seizures; recurrent seizures; status epilepticus seizures, e.g., refractory convulsive status epilepticus, non-convulsive status epilepticus seizures; refractory seizures; myoclonic seizures; tonic seizures; tonic-clonic seizures; simple partial seizures; complex partial seizures; secondarily generalized seizures; atypical absence seizures; absence seizures; atonic seizures; benign Rolandic seizures; febrile seizures; emotional seizures; focal seizures; gelastic seizures; generalized onset seizures; infantile spasms; Jacksonian seizures; massive bilateral myoclonus seizures; multifocal seizures; neonatal onset seizures; nocturnal seizures; occipital lobe seizures; post traumatic seizures; subtle seizures; Sylvan seizures; visual reflex seizures; or withdrawal seizures. In some embodiments, the seizure is a generalized seizure associated with Dravet Syndrome, Lennox-Gastaut Syndrome, Tuberous Sclerosis Complex, Rett Syndrome or PCDH19 Female Pediatric Epilepsy.

### Sterol Synthesis Disorders

In one embodiment, described herein are methods for treating a sterol synthesis disorder. Cholesterol has an essential rule in growth and development. It is a membrane lipid and a precursor to many molecules that play important roles in cellular growth and differentiation, protein glycosylation, and signaling pathways. Biosynthesis of cholesterol involves a number of enzymes and intermediates. Disorders resulting from a deficiency in any of the enzymes involved in cholesterol biosynthesis lead to the accumulation of intermediates and imbalance in biomolecules, resulting in disorders including congenital skeletal malformations, dysmorphic facial features, psychomotor retardation, and failure to thrive. In an embodiment, a sterol synthesis disorder or symptom of a sterol synthesis disorder can be treated by administering to a subject suffering from a sterol synthesis disorder a compound described herein, such as a positive NMDA receptor modulating compound as described herein. Additional disorders are described below.

### Smith-Lemli-Opitz Syndrome

In one embodiment, described herein are methods for treating Smith-Lemli-Opitz Syndrome (or SLOS, or 7-dehydrocholesterol reductase deficiency). SLOS is an inborn error of cholesterol synthesis. In addition to microcephaly, moderate to severe intellectual disability, sensory hypersensitivity, stereotyped behaviors, dysmorphic facial features, and syndactyly of the second/third toes, a feature of the disease is reduced cerebrosterol (24 (S)-hydroxycholesterol) levels. SLOS is an autosomal recessive genetic condition resulting from deficiency in the final enzyme of the cholesterol synthesis pathway, and causes low or low-normal plasma cholesterol levels and increased 7- and 8-dehydrocholesterol (DHC; 7DHC and 8DHC) levels. Common therapies currently used include dietary cholesterol supplementation, treatment with 3-hydroxy-3-methylglutaryl coenzyme A reductase inhibitors (HMG CoA reductase inhibitors, also known as statins), and treatment with agents that enhance cholesterol production and/or accretion; and to decrease the accumulation of 7DHC and 8DHC, the potentially toxic precursors of cholesterol.

### Desmosterolosis

Desmosterolosis is a deficiency in desmosterol reductase and has a similar phenotype to SLOS. In one embodiment, described herein are methods for treating desmosterolosis with compounds described herein.

### Sitosterolemia

Sitosterolemia is a rare autosomal recessive disorder caused by mutations in two ATP-binding cassette (ABC) transporter genes (ABCG5 and ABCG8). Sitosterolemia enhances the absorption of plant sterols and cholesterol from the intestines. Patients typically present with tendon and tuberous xanthomas and premature coronary artery disease. In one embodiment, described herein are methods for treating sitosterolemia with compounds described herein.

### Cerebrotendinous Xanthomatosis ((TX)

In one embodiment, described herein are methods for treating cerebrotendinous xanthomatosis (also referred to as cerebral cholesterosis, or Van Bogaert-Scherer-Epstein syndrome) with compounds described herein. CTX can be caused by a mutation in the CYP27A1 gene, which produces the sterol 27-hydroxylase enzyme. Sterol 27-hydroxylase metabolizes cholesterol into bile acids (e.g., chenodeoxycholic acid) that are important in the absorption of fats in the intestine. Enzyme dysfunction can lead to cholesterol accumulation in tissues. CTX is characterized by childhood diarrhea, cataracts, tendon xanthomas, reduced mental capability and abnormal movements in adults.

### Mevalonate Kinase Deficiency Syndromes (MKD))

Mevalonate Kinase Deficiency (also referred to as mevalonic aciduria (a more severe form of MKD), or Hyper IgD Syndrome (HIDS, or hyperimmunoglobulinemia D) with period fever syndrome (a more benign form of MKD)) causes an accumulation of mevalonic acid in the urine as a result of insufficient activity of mevalonate kinase. MKD can result in developmental delay, hypotonia, anemia, hepatosplenomegaly, dysmorphic features, mental retardation, and overall failure to thrive. Mevalonic aciduria is characterized by delayed physical and mental development, failure to thrive, recurrent episodes of fever with vomiting and diarrhea, enlarged liver, spleen and lymph nodes, microcephaly (small head size), cataract, low muscle tone, short statute, distinct facial features, ataxia, and anemia. HIDS is characterized by recurrent episodes of fever associated with swollen lymph nodes, joint pain, gastrointestinal issues and skin rash. In one embodiment, described herein are methods for treating MKD with the compounds described herein.

### SC4MOL Gene Mutation (SMO Deficiency)

SC4MOL gene deficiency is a genetic disorder in the cholesterol biosynthesis pathway (e.g., mutations in the SC4MOL gene encoding a novel sterol oxidase). SC$MOL deficiency is characterized by the accumulation of dimethyl and monomethyl sterols that can be detected in blood, skin flakes or primary skin fibroblasts. In one embodiment, described herein are methods for treating SMO deficiency with compounds described herein.

### Niemann-Pick Disease

Niemann-Pick disease is a lysosomal storage disease resulting from a genetic mutation that affects metabolism. Niemann-Pick disease leads to abnormal accumulation of cholesterol and other fatty substances (lipids) due to an inability of the body to transport the substances. The accumulation damages the affected areas.

### Autism

In one embodiment, described herein are methods for treating autism spectrum disorder or autism. Autism spectrum disorder (ASD) and autism refer to a group of complex disorders of brain development. Autism is typically characterized by difficulties in social interaction, for example in verbal and nonverbal communication. Repetitive behaviors are also often seen in individuals having autism. Autism can be associated with intellectual disability, difficulties in motor coordination and attention and physical health issues, e.g., sleep and gastrointestinal disturbances. Individuals having autism can also excel in visual skills, music, math and art. Autism can refer to autistic disorder, childhood disintegrative disorder, pervasive developmental disorder-not otherwise specified (PDD-NOS), and Asperger syndrome. Autism also refers to monogenetic causes of autism such as synaptopathies, e.g., Rett syndrome, Fragile X syndrome, Angelman syndrome.

Disorders Associated with Phenylketonuria

In one embodiment, described herein are methods for treating disorders associated with phenylketonuria (e.g., cognitive disorders) with compounds described herein. Phenylketonuria can lead to hypocholesterolemia and lowered vitamin D status. Total and low-density cholesterols and 25-hydroxy vitamin D have been found to be decreased in subjects suffering from phenylketonuria as compared with subjects not suffering from phenylketonuria (Clin. Chim. Acta 2013, 416:54-59). 24S-hydroxycholesterol and 27S-hydroxycholesterol and 7α-hydroxycholesterol (e.g., representing peripheral and hepatic cholesterol elimination, respectively) have been shown to be significantly decreased in subjects suffering from phenylketonuria, while 7β-hydroxycholesterol (e.g., reflecting oxidative stress) was increased significantly in subjects suffering from phenylketonuria. Changes in the levels of 24S-OHC and 7β-hydroxycholesterol correlate with phenylalanine level, and 27S-hydroxycholesterol levels may correlate with the 25-hydroxy vitamin D level in subjects suffering from phenylketonuria.

### Examples

In order that the disclosure described herein may be more fully understood, the following examples are set forth. The synthetic and biological examples described in this application are offered to illustrate the compounds, pharmaceutical compositions and methods provided herein and are not to be construed in any way as limiting their scope.

The absolute configuration of an asymmetric center can be determined using methods known to one skilled in the art. In some embodiments, the absolute configuration of an asymmetric center in a compound can be elucidated from the X-ray single-crystal structure of the compound. In some embodiments, the absolute configuration of an asymmetric center elucidated by the X-ray crystal structure of a compound can be used to infer the absolute configuration of a corresponding asymmetric center in another compound obtained from the same or similar synthetic methodologies. In some embodiments, the absolute configuration of an asymmetric center elucidated by the X-ray crystal structure of a compound can be used to infer the absolute configuration of a corresponding asymmetric center in another compound coupled with a spectroscopic technique, e.g., NMR spectroscopy, e.g., ¹H NMR spectroscopy or ¹⁹F NMR spectroscopy.

### Abbreviations

PE: petroleum ether; CDCl₃: deuterated chloroform; DCM: dichloromethane; EtOAc: ethylacetate; THF: tetrahydrofuran; DMSO: dimethylsulfoxide; DMF: N,N-dimethylformamide; m-CPBA: metachloroperbenzoic acid; NBS: N-bromosuccinimide; DEAD: diethyl azodicarboxylate; FA: formic acid; Me₃SIO: Trimethylsulfoxonium iodide; EtMgBr: Ethylmagnesium Bromide; BH₃: Borane; PCC: pyridinium chlorochromate; t-BuOK: potassium tert-butoxide; 9-BBN: 9-borabicyclo[3.3.1]nonane; BHT: 2,6-di-t-butyl-4-methylphenoxide; Me: methyl; Et: ethyl; i-Pr: iso-propyl; Bu: butyl; 1-Bu: tert-butyl; Ph: phenyl; Bz: benzoyl; BzCl: benzoyl chloride; CsF: cesium fluoride; DMP: Dess-Martin periodinane; TEA: triethylamine; TBAF: tetra-n-butylammonium fluoride; Na₂SO₄: sodium sulfate; Na₂S₂O₃: sodium thiosulfate; NaHCO₃: sodium bicarbonate; MeOH: methanol; EtOH: ethanol; MePPh₃Br: bromo(methyl)triphenylphosphorane; MeMgBr: Methylmagnesium bromide; DMAP: 4-dimethylaminopyridine; Py: pyridine; TMS: trimethylsilyl; TMSCF₃: (trifluoromethyl)trimethylsilane; Ts: p-toluenesulfonyl; LiOH·H₂O: lithium hydroxide hydrates; MeCN: acetonitrile; MAD: methyl aluminum bis(2,6-di-t-butyl-4-methylphenoxide); i-PrMgCl: Isopropylmagnesium chloride; LDA: lithium diisopropylamide; Pd(t-Bu₃P)₂: bis(tri-tert-butylphosphine)palladium (0); AcCl: acetyl chloride; TBSCl: tert-Butyl(chloro)dimethylsilane; (i-PrO)₄Ti: titanium tetraisopropoxide; DCC: dicyclohexylcarbodiimide; AlaOH: alanine; Boc: t-butoxycarbonyl; TBS: t-butyldimethylsilyl; LAH: Lithium Aluminium Hydride; LDA: lithium diisopropylamide; MTBE: methyl tert-butyl ether; MeOD: deuterated methanol; Im: 1H-imidazole; DIPEA: diisopropylethyl amine; (HCHO) n: formaldehyde; TOSMIC; Toluenesulphonylmethyl isocyanide, DMPSCI: chlorodmethylphenylsilane; PCC: pyridinium chlorochromate.

### Materials and Methods

The compounds provided herein can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

The compounds provided herein may be isolated and purified by known standard procedures. Such procedures include (but are not limited to) recrystallization, column chromatography, HPLC, or supercritical fluid chromatography (SFC). The following schemes are presented with details as to the preparation of representative compounds that have been listed herein. The compounds provided herein may be prepared from known or commercially available starting materials and reagents by one skilled in the art of organic synthesis. Exemplary chiral columns available for use in the separation/purification of the enantiomers/diastereomers provided herein include, but are not limited to, CHIRALPAK® AD-10, CHIRALCEL® OB, CHIRALCEL® OB-H, CHIRALCEL® OD, CHIRALCEL® OD-H, CHIRALCEL® OF, CHIRALCEL® OG, CHIRALCEL® OJ and CHIRALCEL® OK.

1H-NMR reported herein (e.g., for the region between δ (ppm) of about 0.5 to about 4 ppm) will be understood to be an exemplary interpretation of the NMR spectrum (e.g., exemplary peak integrations) of a compound. Exemplary general method for preparative HPLC: Column: Waters RBridge prep 10 μm C18, 19*250 mm. Mobile phase: acetonitrile, water (NH₄HCO₃) (30 L water, 24 g NH₄HCO₃, 30 mL NH₃·H₂O). Flow rate: 25 mL/min.

Exemplary general method for analytical HPLC: Mobile phase: A: water (10 mM NH₄HCO₃), B: acetonitrile Gradient: 5%-95% B in 1.6 or 2 min Flow rate: 1.8 or 2 mL/min; Column: XBridge C18, 4.6*50 mm, 3.5 μm at 45 C.

Exemplary general method for SFC: Column: CHIRALPAK® AD CSP (250 mm*30 mm, 10 μm), Gradient: 45% B, A=NH₃H₂O, B=MeOH, flow rate: 60 mL/min. For example, AD_3_EtOH_DEA_5_40_25 ML would indicate: “Column: Chiralpak AD-3 150×4.6 mm I.D., 3 um Mobile phase: A: CO₂ B: ethanol (0.05% DEA) Gradient: from 5% to 40% of B in 5 min and hold 40% for 2.5 min, then 5% of B for 2.5 min Flow rate: 2.5 mL/min Column temp: 35° C.

### Example 1: Synthesis of (3S,5S,8R,9S,10S,13R,14S,17R)-3-ethyl-17-((R)-7-hydroxy-7-methyloctan-3-yl)-10,13-dimethylhexadecahydro-1H-cyclopenta[a]phenanthren-3-ol (A-1)

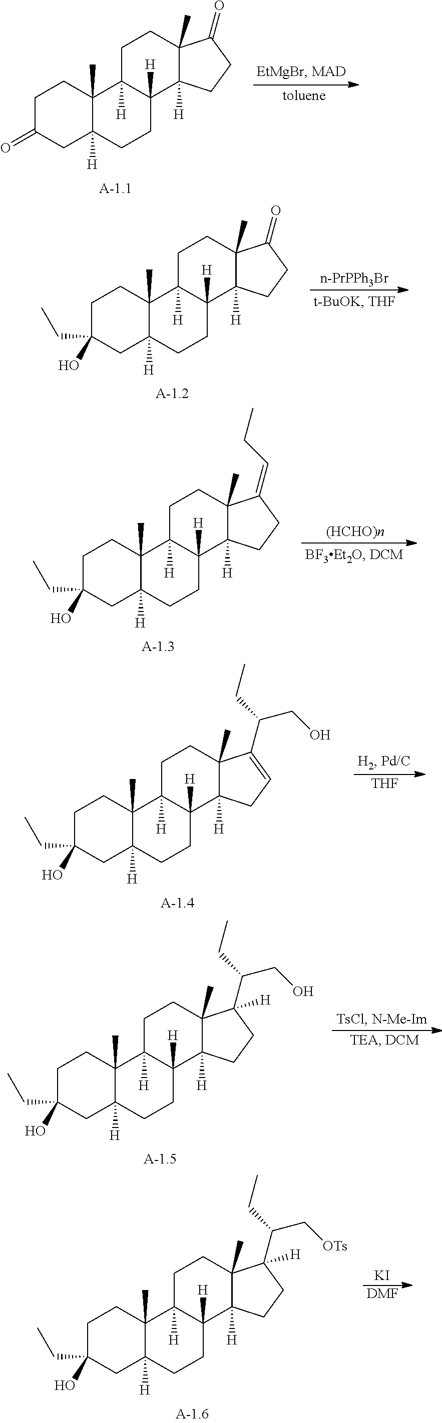

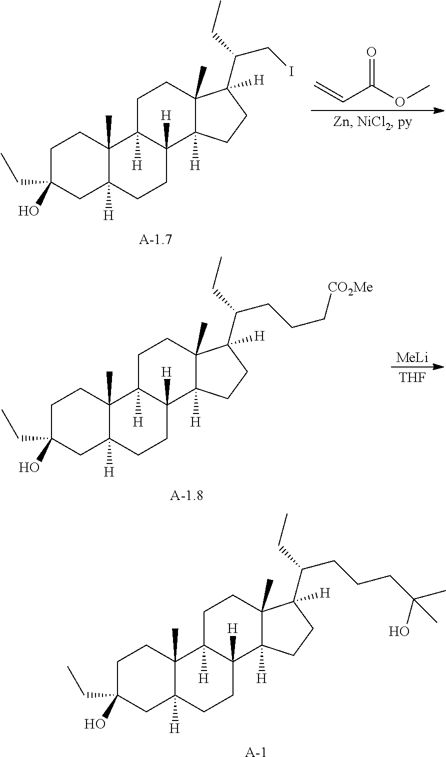

### Synthesis of A-1.2

To a solution of BHT (68.7 g, 312 mmol) in toluene (100 mL) under nitrogen at 0° C. was added trimethylaluminum (2 M in toluene, 78 mL, 156 mmol) dropwise and the mixture was stirred at 25° C. for 1 hour. A solution of A-1.1 (15.0 g, 52 mmol) in DCM (50 mL) was added dropwise at −70° C. and the mixture was stirred for 1 hour. EtMgBr (52 mL, 156 mmol, 3M in ethyl ether) was added dropwise and the mixture was stirred at −70° C. for an additional 2 hours then poured into saturated citric acid (500 mL) at below 10° C. The aqueous phase was extracted with DCM (3×100 mL). The organic phase was washed with brine (2×50 mL), dried over anhydrous Na₂SO₄, filtered, and concentrated. The residue was triturated with MeOH (1.5 L) at 25° C. to give A-1.2 (14.0 g). ¹H NMR (400 MHz, CDCl₃) δ_{H} 2.50-2.38 (m, 1H), 2.12-2.00 (m, 1H), 1.97-1.88 (m, 1H), 1.83-1.74 (m, 2H), 1.70-1.48 (m, 9H), 1.41-1.33 (m, 3H), 1.29-1.23 (m, 3H), 1.21-1.07 (m, 2H), 1.05-0.94 (m, 2H), 0.90-0.81 (m, 9H), 0.78-0.68 (m, 1H).

### Synthesis of A-1.3

To a mixture of n-PrPPh₃Br (36.2 g, 94.0 mmol) in THF (150 mL) was added 1-BuOK (10.5 g, 94.0 mmol) at 20° C. under N₂. The resulting mixture was stirred at 40° C. for 30 min. A-1.2 (15.0 g, 47.0 mmol) in THF (20 mL) was added in portions below 40° C. The reaction mixture was stirred at 40° C. for 16 hours. The reaction mixture was quenched with 10% NH₄Cl (300 mL) at 20° C. and extracted with EtOAc (3×100 mL). The combined organic phase was washed with brine (2×200 mL), filtered, and concentrated. The residue was triturated with MeOH/H₂O (280 ml/280 ml) to give the product which was then purified by flash column chromatography (0˜60% ethyl acetate in PE) to give A-1.3 (11.2 g, 70%). ¹H NMR (400 MHz, CDCl₃) δ_{H} 5.07-4.96 (m, 1H), 2.44-2.28 (m, 1H), 2.24-1.99 (m, 4H), 1.75-1.58 (m, 5H), 1.55-1.20 (m, 10H), 1.17-0.97 (m, 4H), 0.95-0.82 (m, 14H), 0.72-0.66 (m, 1H).

### Synthesis of A-1.4

To a solution of A-1.3 (4.00 g, 11.6 mmol) in DCM (80 mL) was added (HCHO) n (5.21 g, 57.9 mmol) and BF₃·Et₂O (0.29 ml, 2.28 mmol) at 25° C. The mixture was stirred at 25° C. for 2 hours. The reaction mixture was filtered and the filter cake was washed with DCM (2×50 mL). The filtrate was purified by flash column chromatography (0˜30% of EtOAc in PE) to give A-1.4 (2.10 g, 48%).

### Synthesis of A-1.5

To a solution of A-1.4 (5.20 g, 13.8 mmol) in THF (50 mL) was added Pd/C (dry, 10%, 500 mg) under N₂. The suspension was degassed under vacuum, purged with H₂ three times, and stirred under H₂ (50 psi) at 50° C. for 24 hours. The mixture was filtered, washed with THF (2×50 mL), and concentrated. Purification by flash column (0˜30% EtOAc in PE) provided A-1.5 (3.50 g, 67%). ¹H NMR (400 MHz, CDCl₃) δ_{H} 3.71 (dd, J=2.0, 10.8 Hz, 1H), 3.53 (dd, J=4.4, 10.8 Hz, 1H), 1.96-1.91 (m, 1H), 1.81-1.74 (m, 1H), 1.68-1.61 (m, 4H), 1.50-1.33 (m, 13H), 1.23-0.95 (m, 10H), 0.90-0.86 (m, 7H), 0.83 (s, 3H), 0.66 (s, 3H).

### Synthesis of A-1.6

To a solution of A-1.5 (3.70 g, 9.82 mmol) in DCM (40 mL) was added TEA (4.07 ml, 29.4 mmol), N-Me-Imidazole (1.16 ml, 14.7 mmol) and TsCl (2.80 g, 14.7 mmol) and the mixture was stirred at 20° C. for 2 hours. Water was added (100 mL) and the mixture was adjusted to pH ˜3 with HCl (30 mL). The aqueous was extracted with DCM (3×50 mL) and the combined organic layer was washed NaHCO₃ (100 mL), brine (2×100 mL), dried over Na₂SO₄, filtered, and concentrated to give A-1.6 (5.40 g). ¹H NMR (400 MHz, CDCl₃) δ_{H} 7.79 (d, J=8.0 Hz, 2H), 7.34 (d, J=8.0 Hz, 2H), 4.06 (dd, J=2.8, 9.6 Hz, 1H), 3.88 (dd, J=5.2, 9.6 Hz, 1H), 2.45 (s, 3H), 1.95-1.83 (m, 1H), 1.65-1.61 (m, 3H), 1.44-1.30 (m, 8H), 1.19-0.97 (m, 9H), 0.90-0.81 (m, 11H), 0.74-0.58 (m, 9H).

### Synthesis of A-1.7

To a solution of A-1.6 (5.40 g, 10.1 mmol) in DMF (60 mL) was added KI (8.03 g, 48.4 mmol) at 25° C. and the mixture was stirred at 60° C. for 1 hour. The mixture was poured into water (100 mL) and stirred for 20 min. The aqueous phase was extracted with EtOAc (3×50 mL). The combined organic phase was washed with saturated brine (2×200 mL), dried over anhydrous Na₂SO₄, filtered, and concentrated. The residue was purified by silica gel chromatography (PE/EtOAc=0˜15%) to give A-1.7 (3.30 g, 67%). ¹H NMR (400 MHz, CDCl₃) δ_{H} 3.44 (dd, J=2.8, 10.0 Hz, 1H), 3.23 (dd, J=4.0, 9.6 Hz, 1H), 1.97-1.89 (m, 1H), 1.87-1.36 (m, 15H), 1.22-0.92 (m, 9H), 0.91-0.78 (m, 12H), 0.70 (s, 3H), 0.67-0.63 (m, 1H).

### Synthesis of A-1.8

To a mixture of Zn powder (93.4 mg, 1.43 mmol) in dry pyridine (1 mL) was added slowly methyl acrylate (123 mg, 1.43 mmol) and NiCl₂·6H₂O (51.1 mg, 0.31 mmol) at 20° C. under N₂. After stirring at 60° C. for 2 hours, the mixture was cooled to 20° C. and a solution of A-1.7 (100 mg, 0.21 mmol) in pyridine (1 mL) was added slowly. The reaction mixture was stirred for 1.5 hours at 20° C. then diluted with NH₄Cl (20 mL). The mixture was filtered through a pad of silica gel and the filtrate was extracted with EtOAc (2×20 mL). The organic phase was dried over Na₂SO₄, filtered, and concentrated. The residue was purified by flash column (0˜20% of EtOAc in PE) to give A-1.8 (100 mg). ¹H NMR (400 MHz, CDCl₃) δ_{H} 3.66 (s, 3H), 2.35-2.19 (m, 2H), 1.96-1.88 (m, 1H), 1.80-1.59 (m, 7H), 1.51-1.34 (m, 9H), 1.21-0.95 (m, 10H), 0.94-0.85 (m, 5H), 0.84-0.75 (m, 8H), 0.69-0.64 (m, 2H), 0.63 (s, 3H).

### Synthesis of A-1

To a solution of A-1.8 (100 mg, 0.22 mmol) in THF (3 ml) was added methyl lithium (1.39 mL, 2.23 mmol, 1.6 M in Et₂O) dropwise at 0° C. under N₂ and the mixture was stirred at 20° C. for 2 hours. The reaction mixture was quenched by addition of saturated NH₄Cl (30 mL) and extracted with ethyl acetate (3×30 mL). The combined organic phase was washed with brine (2×200 mL), dried over anhydrous Na₂SO₄, filtered and concentrated. The residue was purified by flash column (0˜25% of EtOAc in PE) to give A-1 (20.2 mg, 20%). ¹H NMR (400 MHz, CDCl₃) δ_{H} 1.98-1.87 (m, 1H), 1.77-1.53 (m, 12H), 1.46-1.25 (m, 14H), 1.21 (s, 6H), 1.20-0.91 (m, 8H), 0.88 (t, J=7.6 Hz, 3H), 0.82 (s, 3H), 0.78 (t, J=7.2 Hz, 3H), 0.70-0.59 (m, 4H). LC-ELSD/MS purity>99%, MS ESI calcd for C₃₀H₅₄O₂ [M−2H₂O+H]⁺ 411.5, found 411.5.

### Example 2: Synthesis of (3S,5S,8R,9S,10S,13R,14S,17R)-3-ethyl-10,13-dimethyl-17-((3R,6R)-7,7,7-trifluoro-6-hydroxy-6-methylheptan-3-yl)hexadecahydro-1H-cyclopenta[a]phenanthren-3-ol (A-2)

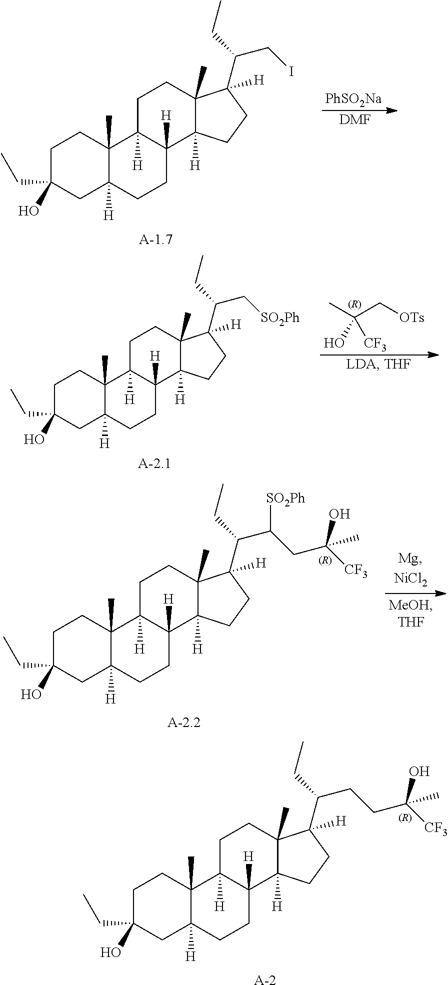

### Synthesis of A-2.1

To a solution of A-1.7 (3.10 g, 6.37 mmol) in DMF (30 mL) was added sodium benzenesulfonate (3.13 g, 19.1 mmol) and the mixture was stirred at 40° C. for 2 hours. The reaction mixture was poured into water (200 mL) to give a precipitate and filtered. The filter cake was washed with water (2×50 mL). The resulting filter cake was dissolved in DCM (200 mL), washed with brine (200 mL), dried over Na₂SO₄, filtered, and concentrated. The residue was purified by flash column (15˜25% EtOAc in PE) to give A-2.1 (2.90 g, 91%). 1H NMR (400 MHz, CDCl₃) δ_{H} 7.96-7.86 (m, 2H), 7.69-7.60 (m, 1H), 7.59-7.53 (m, 2H), 3.08-3.02 (m, 2H), 2.23-2.10 (m, 1H), 1.92-1.80 (m, 1H), 1.78-1.60 (m, 6H), 1.51-1.28 (m, 8H), 1.23-0.93 (m, 10H), 0.90-0.80 (m, 11H), 0.67-0.62 (m, 1H), 0.61 (s, 3H).

### Synthesis of A-2.2

To a solution of A-2.1 (500 mg, 1.00 mmol) in THF (4 mL) at −70° C. under nitrogen was added dropwise n-BuLi (1.59 mL, 3.99 mmol, 2.5M in hexane) at −70° C. The reaction mixture was stirred at −70° C. for 0.5 hour. DIPA (0.560 ml, 3.99 mmol) was added dropwise at -70° C. for 0.5 hour. A solution of (2R)-1,1,1-trifluoro-2-methyl-3-[(4-methylbenzenesulfonyl)oxy]propan-2-ol (384 mg, 1.29 mmol) in THF (1 ml) was added dropwise at -70° C. The reaction mixture was slowly warmed to 25° C. and stirred for 18 hours. The reaction mixture was quenched with saturated NH₄Cl (20 mL) and extracted with EtOAc (2×10 mL). The combined organic phase was washed with brine (50 mL), dried over Na₂SO₄, filtered, and concentrated to give A-2.2 (600 mg) which was used without further purification.

### Synthesis of A-2

To a solution of A-2.2 (0.600 g, 0.96 mmol) in methanol (20 ml) and THF (10 ml) was added nickel (II) chloride (24.6 mg, 0.19 mmol) and Mg powder (916 mg, 38.2 mmol) in four portions under N₂ at 65° C. and the mixture was stirred for 20 mins. The reaction mixture was quenched by addition of citric acid (100 mL, 10%) which was added dropwise until the solid dissolved. After extraction with EtOAc (3×50 mL), the organic layer was washed with sat. NaHCO₃ (100 mL), brine (100 mL), dried over Na₂SO₄, filtered, and concentrated. The residue was purified by flash column (0˜30% of EtOAc in PE) to give A-2 (152.0 mg, 33%). ¹H NMR (400 MHz, CDCl₃) δ_{H} 1.97-1.89 (m, 1H), 1.82-1.56 (m, 9H), 1.55-1.34 (m, 11H), 1.34 (s, 3H), 1.31-0.91 (m, 12H), 0.88 (t, J=7.2 Hz, 3H), 0.84-0.77 (m, 6H), 0.70-0.59 (m, 4H). ¹⁹F NMR (376 MHz, CDCl₃) δ_{F} −82.71 (s). LC-ELSD/MS purity >99%, MS ESI calcd for C₂₉H₄₉F₃O₂ [M−H₂O+H]⁺ 469.4, found 469.4.

### Example 3: Synthesis of (3S,5S,8R,9S,10S,13R,14S,17R)-3-ethyl-10,13-dimethyl-17-((3R,6S)-7,7,7-trifluoro-6-hydroxy-6-methylheptan-3-yl)hexadecahydro-1H-cyclopenta[a]phenanthren-3-ol (A-3)

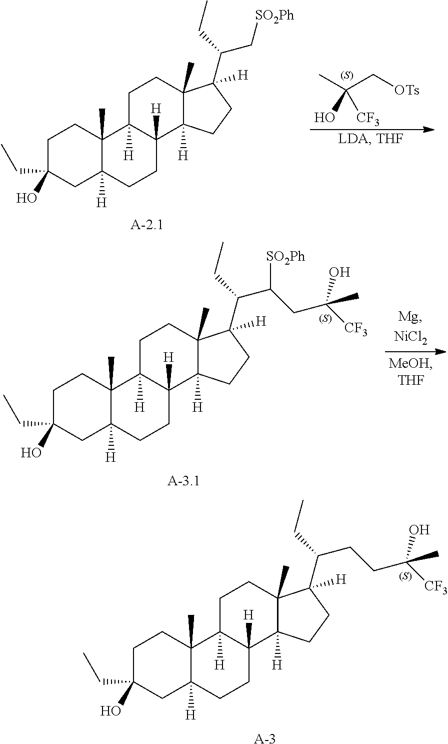

### Synthesis of A-3.1

To a solution of A-2.1 (500 mg, 1.00 mmol) in THF (4 mL) at −70° C. under N₂ at 70° C. was added dropwise a solution of n-BuLi (1.59 mL, 3.99 mmol, 2.5M in hexane). The reaction mixture was stirred at -70° C. for 0.5 hour. DIPA (0.56 ml, 3.99 mmol) was added dropwise at −70° C. for 0.5 h. (2S)-1,1,1-trifluoro-2-methyl-3-[(4-methylbenzenesulfonyl)oxy]propan-2-ol (297 mg, 1.00 mmol) was added dropwise at −70° C. The reaction mixture was warmed to 25° C. slowly, and stirred at 25° C. for 18 hours. The reaction mixture was quenched with saturated NH₄Cl (20 mL) and extracted with EtOAc (2×10 mL). The combined organic phase was washed with 10% NH₄Cl (30 mL), dried over Na₂SO₄, filtered, and concentrated under vacuum to give A-3.1 (600 mg) which was used in the next step without further purification.

### Synthesis of A-3

To a solution of A-3.1 (0.600 g, 0.96 mmol) in methanol (20 ml) and THF (10 ml) was added nickel (II) chloride (24.6 mg, 0.19 mmol) and Mg powder (916 mg, 38.2 mmol) in four portions under N₂ at 65° C. and the mixture was stirred for 20 mins. The reaction mixture was quenched by addition of citric acid (100 mL, 10%), which was added dropwise until the solid dissolved. The mixture was extracted with EtOAc (3×50 mL), the organic layer was washed with sat. NaHCO₃ (100 mL), brine (100 mL), dried over Na₂SO₄, filtered, and concentrated. The residue was purified by flash column (0˜30% of EtOAc in PE) to give A-3 (21.4 mg, 5%). ¹H NMR (400 MHz, CDCl₃) δ_{H} 1.97-1.88 (m, 1H), 1.80-1.55 (m, 11H), 1.52-1.36 (m, 8H), 1.33 (s, 3H), 1.31-0.91 (m, 13H), 0.88 (t, J=7.2 Hz, 3H), 0.82 (s, 3H), 0.82-0.77 (m, 3H), 0.69-0.60 (m, 4H). ¹⁹F NMR (376 MHz, CDCl₃) δ_{F} −82.99 (s). LC-ELSD/MS purity >99%, MS ESI calcd for C₂₉H₄₉F₃O₂ [M−H₂O+H]⁺ 469.4, found 469.4.

### Example 4: Synthesis of (3S,5S,8R,9S,10S,13S,14S,17S)-3-ethyl-17-((1R,4S)-1-fluoro-4-hydroxy-5-methylhexyl)-10,13-dimethylhexadecahydro-1H-cyclopenta[a]phenanthren-3-ol (A-4) and (3S,5S,8R,9S,10S,13S,14S,17S)-3-ethyl-17-((1R,4R)-1-fluoro-4-hydroxy-5-methylhexyl)-10,13-dimethylhexadecahydro-1H-cyclopenta[a]phenanthren-3-ol (A-5)

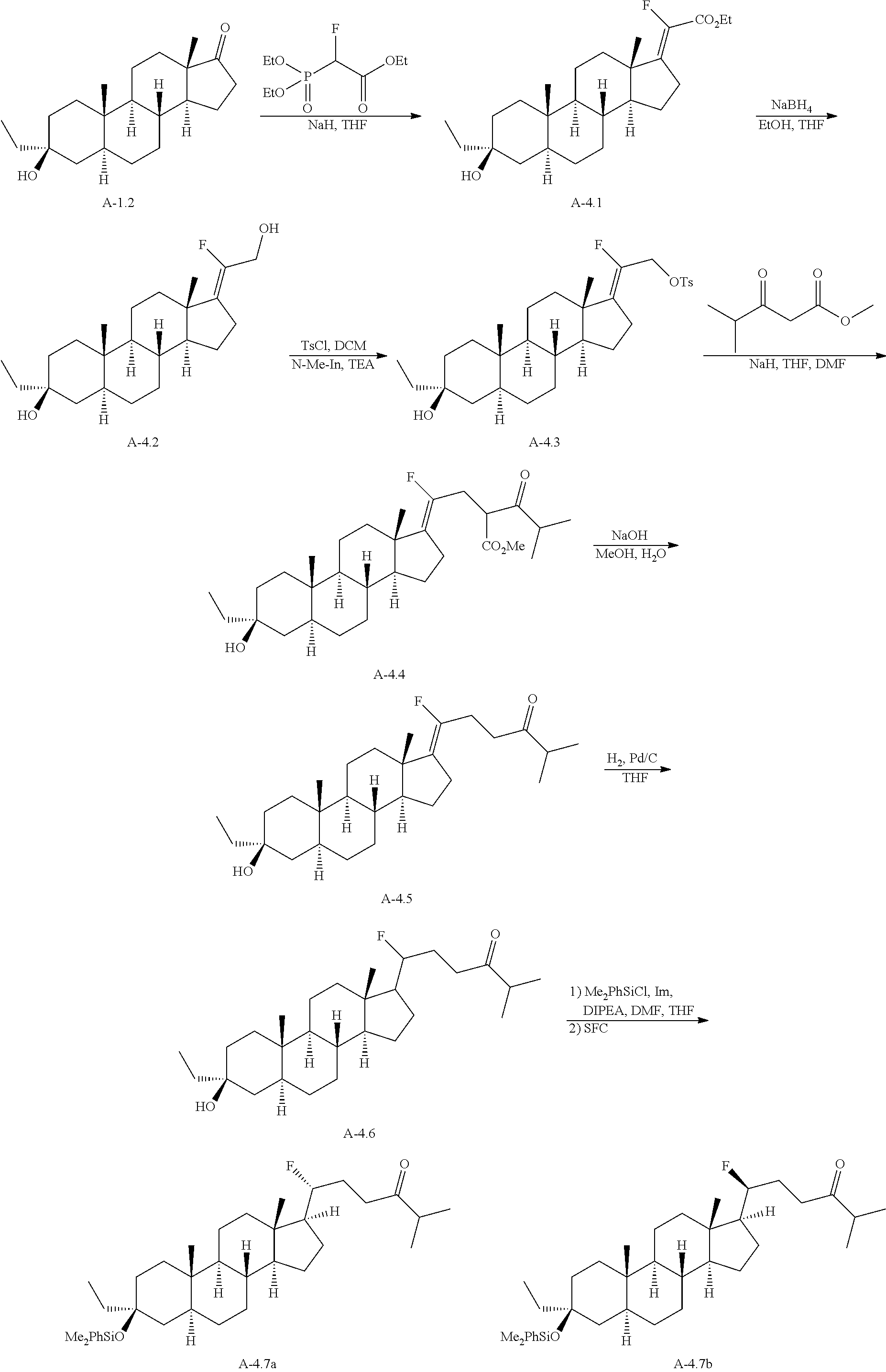

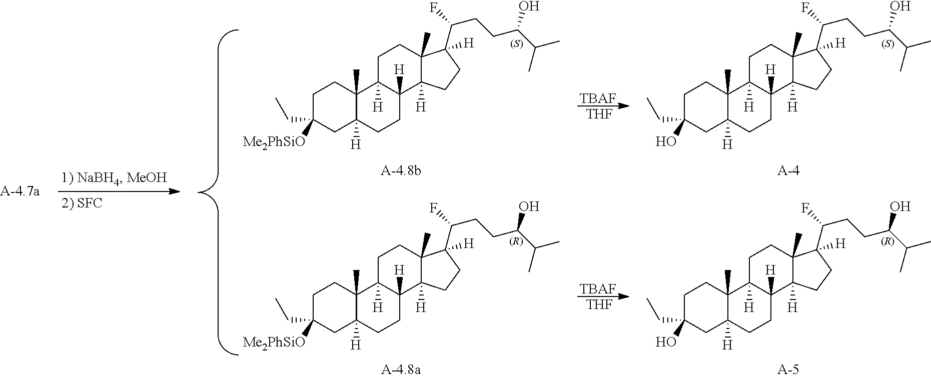

### Synthesis of A-4.1

To a suspension of NaH (1.37 g, 60%, 34.3 mmol) in THF (20 mL) was added ethyl 2-(diethoxyphosphoryl)-2-fluoroacetate (9.44 g, 39.0 mmol) at 0° C. under N₂. To this mixture was added a solution of A-1.2 (5.00 g, 15.6 mmol) in THF (30 mL) dropwise and the reaction mixture was stirred at 60° C. for 16 hours. The mixture was poured into saturated NH₄Cl (100 mL) and the aqueous phase was extracted with EtOAc (2×100 mL). The combined organic phase was washed with water (2×100 mL), dried over anhydrous Na₂SO₄, filtered, and concentrated. The residue was purified by flash column (0˜15% of EtOAc in PE) to give A-4.1 (3.50 g, 55%). ¹H NMR (400 MHz, CDCl₃) δ_{H} 4.33-4.18 (m, 2H), 2.68-2.26 (m, 3H), 1.82-1.34 (m, 14H), 1.30-1.07 (m, 9H), 0.99 (s, 3H), 0.95-0.85 (m, 5H), 0.84 (s, 3H).

### Synthesis of A-4.2

To a solution of A-4.1 (7.50 g, 18.4 mmol) in THF (3.0 mL) and EtOH (5.0 mL) was added NaBH₄ (9.04 g, 239 mmol) at 25° C. under N₂ and the mixture was stirred for 48 hours. The reaction mixture was quenched by addition of saturated NH₄Cl (200 mL). The aqueous layer was extracted with EtOAc (2×50 mL) and the combined organic phase was concentrated. The residue was purified by flash column (0˜20% of EtOAc in PE) to give A-4.2 (5.00 g, 75%). ¹H NMR (400 MHz, CDCl₃) δ_{H} 4.34-4.11 (m, 2H), 2.49-2.22 (m, 2H), 2.04-1.98 (m, 1H), 1.75-1.50 (m, 9H), 1.47-0.88 (m, 19H), 0.84 (s, 3H), 0.74-0.65 (m, 1H).

### Synthesis of A-4.3

To a solution of A-4.2 (5.00 g, 13.7 mmol) in DCM (50 mL) was added N-methylimidazole (2.17 mL, 27.4 mmol), TEA (5.67 mL, 41.0 mmol), and TsCl (3.90 g, 20.5 mmol). The mixture was stirred at 25° C. for 1 hour and then poured into saturated NaHCO₃ (50 mL). The aqueous phase was extracted with DCM (2×50 mL). The combined organic phase was washed with brine (50 mL), dried over anhydrous Na₂SO₄, filtered, and concentrated. The residue was purified by column chromatography (15˜30% EtOAc in PE) to give A-4.3 (3.00 g, 57%). ¹H NMR (400 MHz, CDCl₃) δ_{H} 4.28-4.08 (m, 2H), 2.53-2.24 (m, 2H), 2.04-1.96 (m, 1H), 1.77-1.60 (m, 5H), 1.51-0.94 (m, 16H), 0.90-0.86 (m, 6H), 0.84 (s, 3H), 0.78-0.66 (m, 1H).

### Synthesis of A-4.4

To a suspension of NaH (363 mg, 9.10 mmol) in THF (5.0 mL) was added methyl-4-methyl-3-oxopentanoate (1.68 g, 11.7 mmol) at 25° C. under N₂ followed by DMF (5 mL). To this mixture, a solution of A-4.3 (500 mg, 1.30 mmol) in THF (5.0 mL) was added dropwise and the reaction mixture was stirred at 60° C. for 24 hours. The mixture was poured into saturated NH₄Cl (20 mL) and the aqueous phase was extracted with EtOAc (2×20 mL). The combined organic phase was washed with water (2×20 mL), dried over anhydrous Na₂SO₄, filtered, and concentrated. The residue was purified by flash column (8˜16% of EtOAc in PE) to give A-4.4 (480 mg, 75%). ¹H NMR (400 MHz, CDCl₃) δ_{H} 4.05-3.93 (m, 1H), 3.71 (s, 3H), 2.98-2.00 (m, 6H), 1.73-1.51 (m, 7H), 1.44-1.20 (m, 9H), 1.15-1.06 (m, 8H), 0.95-0.68 (m, 11H).

### Synthesis of A-4.5

To a mixture of A-4.4 (480 mg, 0.9781 mmol) in MeOH (9.0 mL) was added H₂O (3 mL) and NaOH (391 mg, 9.78 mmol) at 25° C. followed by stirring at 60° C. for 16 hours. The reaction mixture was concentrated and H₂O (20 mL) was added. The mixture was extracted with EtOAc (3×30 mL) and the combined organic phases were washed with brine (15 mL), dried over anhydrous Na₂SO₄, filtered, and concentrated to give the product A-4.5 (400 mg, 95%). ¹H NMR (400 MHz, CDCl₃) δ_{H} 2.71-2.30 (m, 7H), 2.06-1.99 (m, 1H), 1.74-1.50 (m, 9H), 1.36-1.08 (m, 16H), 1.01-0.93 (m, 2H), 0.90-0.82 (m, 9H), 0.74-0.62 (m, 1H).

### Synthesis of A-4.6

To a solution of A-4.5 (1.60 g, 3.69 mmol) in THF (20 mL) was added Pd/C (200 mg, dry) and the mixture was stirred at 40° C. under H₂ (40 Psi) for 48 hours. The mixture was then filtered and concentrated to give A-4.6 (1.00 g) that was carried into the next step without further purification.

### Synthesis of A-4.7a and A-4.7b

To a solution of A-4.6 (1.20 g, 2.76 mmol) in THF/DMF (12 ml, 1:1) was added 1H-imidazole (748 mg, 11.0 mmol), DIPEA (1.42 g, 11.0 mmol), and chlorodimethylphenylsilane (942 mg, 5.52 mmol) and the reaction mixture was stirred at 25° C. for 18 hours. The mixture was poured into aqueous saturated NaHCO₃ (20 mL) and then extracted with EtOAc (3×20 mL). The combined organic phase was washed with brine (20 mL), dried over anhydrous Na₂SO₄, filtered, and concentrated. The product was then purified by flash column (0˜5% of EtOAc in PE) and separated by SFC (Column: DAICEL CHIRALPAK AD (250 mm*50 mm, 10 um); Conditions: 0.1% NH₃H₂O in EtOH; Begin B: 25%; End B: 25%; FlowRate (ml/min): 200; Injections: 80) to give A-4.7a (640 mg, 64%) and A-4.7b (160 mg, 16%). The obtained A-4.7a (640 mg, 1.12 mmol) was further purified by SFC (Column: DAICEL CHIRALCEL OD (250 mm*30 mm, 10 um); Condition: 0.1% NH₃H₂O in EtOH; Begin B: 25%; End B: 25%; FlowRate (ml/min): 60; Injections: 80) to give A-4.7a (130 mg, 20.4%).

A-4.7a: ¹H NMR (400 MHz, CDCl₃) δ_{H} 7.64-7.56 (m, 2H), 7.37-7.31 (m, 3H), 4.51-4.27 (m, 1H), 2.74-2.51 (m, 3H), 2.00-1.80 (m, 2H), 1.62-1.15 (m, 18H), 1.12-1.08 (m, 7H), 1.07-0.90 (m, 4H), 0.88-0.82 (m, 4H), 0.72-0.67 (m, 6H), 0.66-0.58 (m, 1H), 0.37 (s, 6H). ¹⁹F NMR (376 MHz, CDCl₃) δ_{F} −180.80 (s).

A-4.7b: ¹H NMR (400 MHz, CDCl₃) δ_{H} 7.64-7.57 (m, 2H), 7.38-7.30 (m, 3H), 4.65-4.22 (m, 1H), 2.67-2.57 (m, 3H), 2.04-1.56 (m, 11H), 1.50-1.13 (m, 10H), 1.11-1.08 (m, 6H), 1.06-0.89 (m, 4H), 0.88-0.82 (m, 4H), 0.68 (s, 3H), 0.67-0.57 (m, 4H), 0.37 (s, 6H). ¹⁹F NMR (376 MHz, CDCl₃) δ_{F} −185.29 (s).

### Synthesis of A-4.8a and A-4.8b

To a solution of A-4.7a (110 mg, 0.1933 mmol) in MeOH (5.0 mL) was added NaBH₄ (219 mg, 5.79 mmol) at 25° C. under N₂ and the reaction mixture was stirred at 25° C. for 0.5 hour. The reaction mixture was quenched by addition of saturated NH₄Cl (10 mL) at 25° C. and the aqueous layer was extracted with EtOAc (2×10 mL). The combined organic phase was concentrated, purified by flash column (0˜5% of EtOAc in PE), and further separated by SFC (Column: DAICEL CHIRALPAK IG (250 mm*30 mm, 10 um); Condition: 0.1% NH₃H₂O; Begin B: 35%; End B: 35%; FlowRate (ml/min): 70; Injections: 30) to give A-4.8a (60.0 mg, 60%) and A-4.8b (30.0 mg, 30%).

A-4.8a: ¹H NMR (400 MHz, CDCl₃) δ_{H} 7.66-7.55 (m, 2H), 7.37-7.32 (m, 3H), 4.63-4.29 (m, 1H), 3.49-3.25 (m, 1H), 2.00-1.88 (m, 1H), 1.72-1.63 (m, 4H), 1.59-1.42 (m, 10H), 1.40-0.96 (m, 13H), 0.94-0.90 (m, 7H), 0.88-0.82 (m, 4H), 0.73 (s, 3H), 0.69 (s, 3H), 0.66-0.58 (m, 1H), 0.37 (s, 6H). ¹⁹F NMR (376 MHz, CDCl₃) δ_{F} −179.51 (s).

A-4.8b: ¹H NMR (400 MHz, CDCl₃) δ_{H} 7.65-7.57 (m, 2H), 7.37-7.32 (m, 3H), 4.64-4.29 (m, 1H), 3.44-3.29 (m, 1H), 2.02-1.90 (m, 1H), 1.84-1.63 (m, 4H), 1.58-1.42 (m, 11H), 1.40-1.01 (m, 12H), 0.94-0.89 (m, 7H), 0.88-0.82 (m, 4H), 0.72 (s, 3H), 0.69 (s, 3H), 0.66-0.58 (m, 1H), 0.37 (s, 6H). ¹⁹F NMR (376 MHz, CDCl₃) δ_{F} −178.77 (s).

### Synthesis of A-5

To a solution of A-4.8a (60.0 mg, 0.1050 mmol) in THF (2 mL) was added TBAF (1.05 mL, 1 M in THF, 1.05 mmol) and the mixture was stirred at 25° C. for 16 hours. The mixture was quenched with H₂O (10 mL) and extracted with EtOAc (2×10 mL). The combined organics were dried over anhydrous Na₂SO₄, filtered and concentrated. The residue was purified by flash column chromatography (15-30% of EtOAc in PE) to give A-5 (50.0 mg, 98%). The A-5 (45.0 mg, 0.1030 mmol) was further lyophilized to give A-5 (19.8 mg, 44%). ¹H NMR (400 MHz, CDCl₃) δ_{H} 4.57-4.37 (m, 1H), 3.41-3.33 (m, 1H), 2.01-1.93 (m, 1H), 1.71-1.57 (m, 13H), 1.42-0.99 (m, 16H), 0.92, (d, J=6.8 Hz, 6H), 0.90-0.85 (m, 4H), 0.83 (s, 3H), 0.74 (s, 3H), 0.71-0.63 (m, 1H). ¹⁹F NMR (376 MHz, CDCl₃) δ_{F} −179.53 (s). LC-ELSD/MS purity >99%; MS ESI calcd for C₂₈H₄₉FO₂ [M+H−HF]⁺ 417.4, found 417.4.

### Synthesis of A-4

To a solution of A-4.8b (25.0 mg, 0.04378 mmol) in THF (2.0 mL) was added TBAF (0.875 mL, 1 M in THF, 0.875 mmol) and the mixture was stirred at 25° C. for 16 hours. The mixture was quenched with H₂O (10 mL), extracted with EtOAc (2×10 mL), dried over anhydrous Na₂SO₄, filtered, and concentrated. Purification by flash column (15-30% of EtOAc in PE) provided A-4 (25.0 mg), which was further lyophilized to give A-4 (21.5 mg, 86%). ¹H NMR (400 MHz, CDCl₃) δ_{H} 4.58-4.31 (m, 1H), 3.42-3.31 (m, 1H), 2.01-1.91 (m, 1H), 1.82-1.57 (m, 11H), 1.43-0.96 (m, 18H), 0.95-0.85 (m, 10H), 0.83 (s, 3H), 0.74 (s, 3H), 0.70-0.63 (m, 1H). ¹⁹F NMR (376 MHz, CDCl₃) δ_{F} −178.77 (s). LC-ELSD/MS purity >99%; MS ESI calcd for C₂₈H₄₉FO₂ [M+H−H₂O−HF]⁺ 399.4, found 399.4.

### Example 5: Synthesis of (3S,5S,8R,9S,10S,13S,14S,17S)-3-ethyl-17-((1R,4R)-4-hydroxy-1-methoxy-5-methylhexyl)-10,13-dimethylhexadecahydro-1H-cyclopenta[a]phenanthren-3-ol (A-6) and (3S,5S,8R,9S,10S,13S,14S,17S)-3-ethyl-17-((1R,4S)-4-hydroxy-1-methoxy-5-methylhexyl)-10,13-dimethylhexadecahydro-1H-cyclopenta[a]phenanthren-3-ol (A-7)

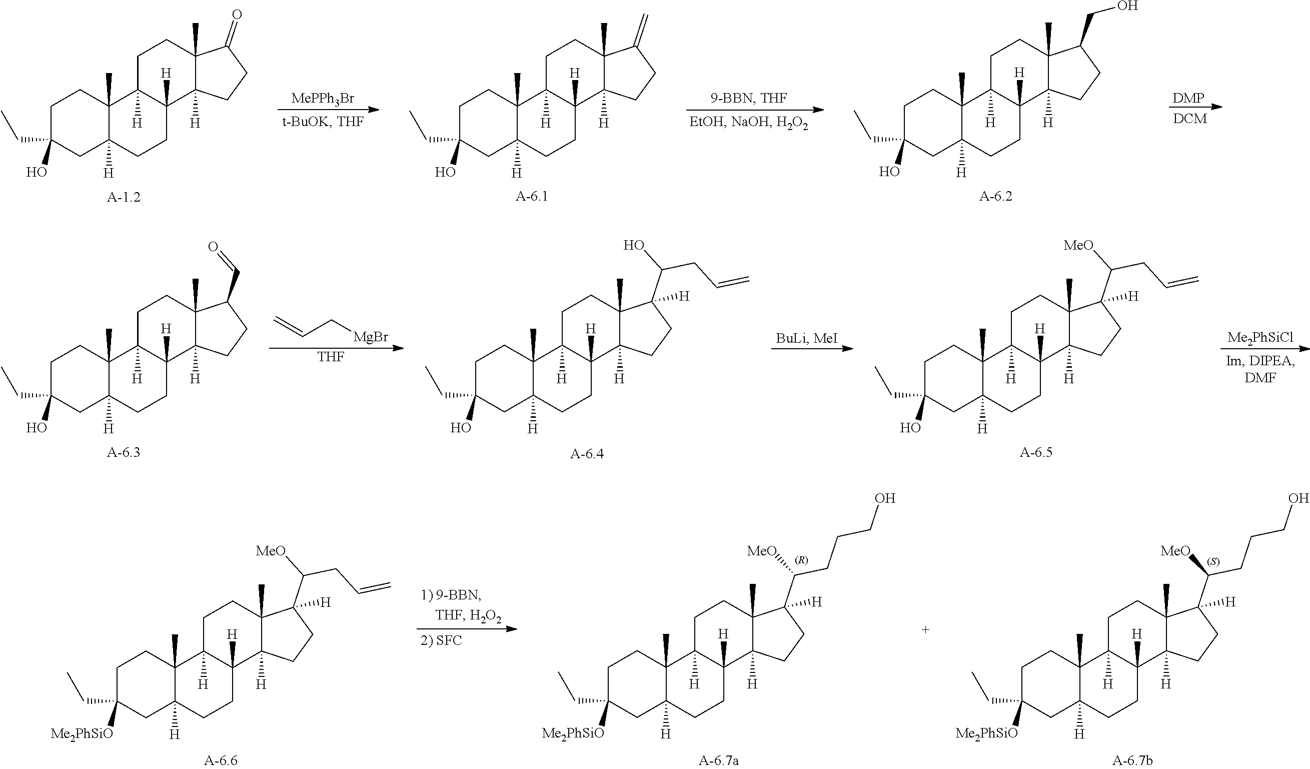

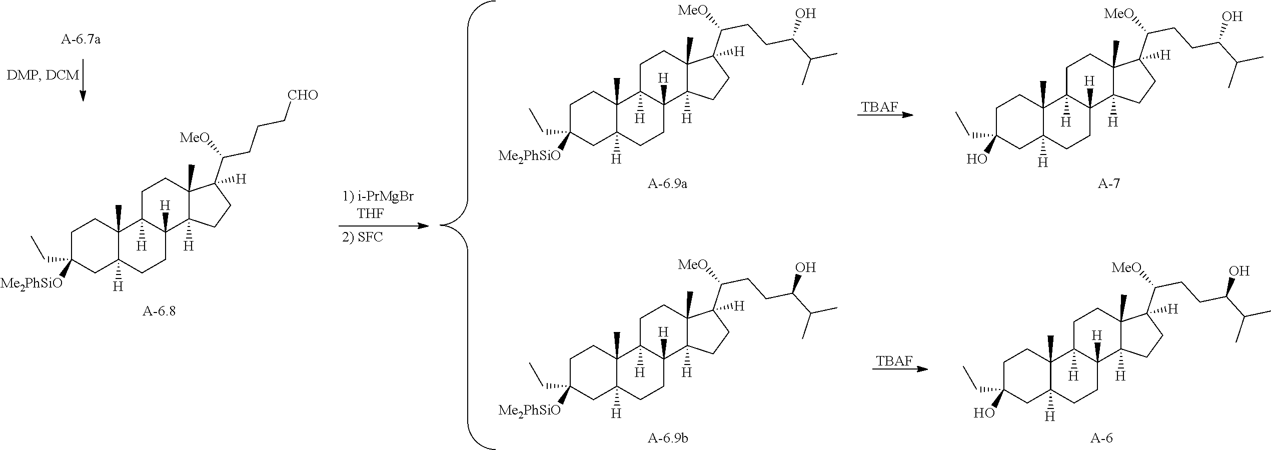

### Synthesis of A-6.1

To a suspension of Ph₃PMeBr (174 g, 489 mmol) in THF (800 mL) was added t-BuOK (54.7 g, 489 mmol) at 25° C. under N₂. After stirring for 30 mins, a solution of A-1.2 (52.0 g, 163 mmol) in THF (700 mL) was added drop-wise. After stirring for 3 hours under N₂, the reaction mixture was poured into MeOH/H₂O (1200 mL/1200 mL). The mixture was extracted with DCM, dried over anhydrous Na₂SO₄, filtered, and concentrated to give A-6.1 (64.0 g) that was carried into the next step without further purification. ¹H NMR (400 MHz, CDCl₃)) δ_{H} 4.65-4.55 (m, 2H), 2.56-2.40 (m, 1H), 2.29-2.14 (m, 1H), 1.88-1.66 (m, 5H), 1.64-1.61 (m, 1H), 1.58-1.48 (m, 3H), 1.44-1.34 (m, 3H), 1.32-1.06 (m, 7H), 1.05-0.91 (m, 3H), 0.90-0.83 (m, 6H), 0.76 (s, 3H), 0.72-0.61 (m, 1H).

### Synthesis of A-6.2

To a solution of A-6.1 (64.0 g, 202 mmol) in THF (600 mL) was added 9-BBN dimer (54.1 g, 222 mmol) at 25° C. under N₂. After stirring for 16 hours, NaOH (280 mL, 5 M, 1.41 mol) was added very slowly followed by careful addition of H₂O₂ (140 mL, 10M, 1.41 mol) until the inner temperature no longer rises and the inner temperature was maintained below 30° C. The mixture was slowly poured into Na₂S₂O₃ (2 L, sat.) to give a precipitate. The mixture was filtered and concentrated under vacuum to give A-6.2 (50.0 g), which was carried into the next step without further purification. ¹H NMR (400 MHz, CDCl₃) δ_{H} 3.86-3.64 (m, 1H), 3.60-3.43 (m, 1H), 1.95-1.71 (m, 8H), 1.36-1.20 (m, 12H), 1.06-0.92 (m, 4H), 0.91-0.79 (m, 8H), 0.76-0.24 (m, 4H).

### Synthesis of A-6.3

To a solution of A-6.2 (47.0 g, 140 mmol) in DCM (800 mL) was added DMP (118 g, 280 mmol). After stirring at 25° C. for 10 mins, the mixture was poured into Na₂S₂O₃/NaHCO₃ (800 mL/800 mL, aq.) and extracted with DCM (2×500 mL). The organic layer was washed with Na₂S₂O₃ (500 mL, sat.), brine (500 mL, sat.), dried over Na₂SO₄, filtered, and concentrated. The residue was combined with another batch of A-6.2 (3.00 g) to be purified by flash column (0˜20% of EtOAc in PE) to give A-6.3 (45.0 g). ¹H NMR (400 MHz, CDCl₃) δ_{H} 9.75 (d, J 2.0 Hz, 1H), 2.20-2.30 (m, 1H), 2.14-2.04 (m, 1H), 2.00-1.94 (m, 1H), 1.79-1.65 (m, 4H), 1.60-1.51 (m, 5H), 1.44-1.30 (m, 5H), 1.29-1.18 (m, 4H), 1.16-0.92 (m, 4H), 0.87 (t, J=7.4 Hz, 3H), 0.83 (s, 3H), 0.76-0.66 (m, 4H).

### Synthesis of A-6.4

To a solution of A-6.3 (10.0 g, 30 mmol) in THF (100 mL) was slowly added bromo (prop-2-en-1-yl) magnesium (90 mL, 90 mmol, 1M) at 25° C. under N₂. After stirring for 2 hours, the mixture was poured into 10% NH₄Cl (300 mL) and extracted with EtOAc (2×100 mL). The combined organic phase was washed with 10% NH₄Cl (300 mL), dried over Na₂SO₄, filtered, and concentrated. The residue was purified by flash column (0˜20% of EtOAc in PE) to give A-6.4 (7.50 g, 67%).

### Synthesis of A-6.5

To a solution of A-6.4 (7.50 g, 20 mmol) in THF (70 mL) was slowly added BuLi (5.56 mL, 13.9 mmol) at 0° C. under N₂. The mixture was stirred at 25° C. for 1 hour. Mel (28.3 g, 200 mmol) was added at 0° C. and then stirring was continued at 25° C. for 16 hours. The mixture was poured into saturated NH₄Cl (100 mL) and the aqueous layer was extracted with EtOAc (2×100 mL). The combined organic phase was washed with brine (200 mL), dried over Na₂SO₄, filtered, and concentrated. The residue was purified by silica gel chromatography (15˜20% EtOAc in PE) to provide A-6.5 (500 mg, 6.4%). ¹H NMR (400 MHz, CDCl₃) δ_{H} 5.92-5.75 (m, 1H), 5.13-4.99 (m, 2H), 3.31 (s, 3H), 3.19-3.11 (m, 1H), 2.44-2.34 (m, 1H), 2.29-2.19 (m, 1H), 2.08-1.97 (m, 1H), 1.86-1.59 (m, 7H), 1.54-1.17 (m, 12H), 1.14-0.94 (m, 5H), 0.88 (t, J 7.4 Hz, 3H), 0.83 (s, 3H), 0.70-0.60 (m, 4H).

### Synthesis of A-6.6

To a solution of A-6.5 (4.10 g, 10.5 mmol) in DMF (50 ml) was added 1H-imidazole (2.14 g, 31.5 mmol), DIPEA (4.06 g, 31.5 mmol), and chlorodimethylphenylsilane (7.16 g, 42 mmol), and the mixture was stirred at 25° C. for 16 hours. The mixture was poured into aqueous saturated NaHCO₃ (100 mL) and extracted with DCM (3×50 mL). The combined organic phase was washed with brine (100 mL), dried over anhydrous Na₂SO₄, filtered, and concentrated. The residue was purified by flash column (0˜20% of EtOAc in PE) to give A-6.6 (4.20 g, 77%).

### Synthesis of A-6.7a and A-6.7b

To a solution of A-6.6 (5.50 g, 10.5 mmol) in THF (60 mL) was added 9-BBN dimer (5.08 g, 21 mmol) at 25° C. under N₂. After stirring at 25° C. for 16 hours, to the reaction was added EtOH (7.33 mL, 126 mmol) and NaOH (25.2 mL, 5 M, 126 mmol) very slowly. Next H₂O₂ (12.6 mL, 10M, 126 mmol) was added carefully such that the mixture was maintained below 30° C. The mixture was stirred at 25° C. for an additional 30 minutes and then slowly poured into 10% Na₂SO₃ (200 mL). The aqueous layer was extracted with EtOAc (3×100 mL) and the combined organics were washed with brine (200 mL), dried over anhydrous Na₂SO₄, filtered, and concentrated. The residue was purified by silica gel column (EtOAc in PE, 0%˜50%), and then further purified by SFC (Column: DAICEL CHIRALPAK AD (250 mm*50 mm, 10 um); Condition: 0.1% NH₃H₂O in EtOH; Begin B: 25%; End B: 25%; FlowRate (ml/min): 210; Injections: 120) to provide A-6.7b (2.70 g, 56%) and A-6.7a (1.10 g, 23%).

A-6.7b: ¹H NMR (400 MHz, CDCl₃) δ_{H} 7.63-7.57 (m, 2H), 7.37-7.31 (m, 3H), 3.69-3.55 (m, 2H), 3.31 (s, 3H), 3.18-3.09 (m, 1H), 2.18-2.14 (m, 1H), 1.92-1.61 (m, 10H), 1.55-0.88 (m, 18H), 0.85 (t, J 7.4 Hz, 3H), 0.68 (s, 3H), 0.65-0.56 (m, 4H), 0.37 (s, 6H).

A-6.7a: ¹H NMR (400 MHz, CDCl₃) δ_{H} 7.64-7.56 (m, 2H), 7.36-7.32 (m, 3H), 3.70-3.51 (m, 2H), 3.30 (s, 3H), 3.28-3.20 (m, 1H), 2.48-2.39 (m, 1H), 2.02-1.93 (m, 1H), 1.80-1.57 (m, 10H), 1.54-0.89 (m, 17H), 0.85 (t, J 7.4 Hz, 3H), 0.69 (s, 3H), 0.66 (s, 3H), 0.65-0.57 (m, 1H), 0.37 (s, 6H).

### Synthesis of A-6.8

To a solution of A-6.7a (0.500 g, 0.9243 mmol) in DCM (10 mL) was added DMP (780 mg, 27.4 mmol) and the mixture was stirred at 25° C. for 1 hour. After 1 hour, NaHCO₃ (20 mL, sat.) and Na₂S₂O₃ (20 mL, sat.) were added sequentially. The aqueous phase was extracted with DCM (2×20 mL) and the combined organic phases were washed with NaHCO₃/Na₂S₂O₃ (1:1, 50 mL, sat.), brine (50 mL), dried over anhydrous Na₂SO₄, filtered, and concentrated to give A-6.8 (500 mg). ¹H NMR (400 MHz, CDCl₃) δ_{H} 9.79 (s, 1H), 7.64-7.57 (m, 2H), 7.37-7.31 (m, 3H), 3.34-3.20 (m, 4H), 2.62-2.38 (m, 2H), 2.05 (s, 3H), 1.65-1.59 (m, 5H), 1.51-0.97 (m, 16H), 0.90-0.80 (m, 5H), 0.74-0.54 (m, 7H), 0.37 (m, 6H).

### Synthesis of A-6.9a and A-6.9b

A mixture of i-PrMgBr (1.39 mL, 2M, 2.78 mmol) in THF (5 mL) was reacted with A-6.8 (500 mg, 0.9278 mmol) in THF (5 mL). After stirring at 0° C. for 1 hour, the reaction mixture was quenched with saturated NH₄Cl (50 mL) at 0° C. and the mixture was extracted with DCM (3×50 mL). The combined organic layers were dried over Na₂SO₄ filtered, and concentrated. The residue was purified by a silica gel column chromatography (EtOAc in PE, 0˜30%) and further purified by SFC (Column: DAICEL CHIRALPAK AD (250 mm*30 mm, 10 um); Conditions: 0.1% NH₃H₂O in IPA; Begin B: 20%; End B: 20%; FlowRate (ml/min): 60; Injections: 70) to provide A-6.9a (110 mg) and A-6.9b (140 mg).

A-6.9a: ¹H NMR (400 MHz, CDCl₃) δ_{H} 7.66-7.55 (m, 2H), 7.39-7.30 (m, 3H), 3.31 (s, 3H), 3.29-3.21 (m, 2H), 2.68 (d, J 3.6 Hz, 1H), 2.00-1.93 (m, 1H), 1.90-1.82 (m, 1H), 1.70-1.58 (m, 6H), 1.54-0.96 (m, 19H), 0.94-0.89 (m, 7H), 0.89-0.80 (m, 4H), 0.69 (s, 3H), 0.65 (s, 3H), 0.63-0.54 (m, 1H), 0.37 (s, 6H).

A-6.9b: ¹H NMR (400 MHz, CDCl₃) δ_{H} 7.64-7.57 (m, 2H), 7.38-7.30 (m, 3H), 3.33-3.22 (m, 5H), 2.03-1.93 (m, 1H), 1.68-1.57 (m, 9H), 1.54-0.98 (m, 18H), 0.95-0.90 (m, 7H), 0.90-0.80 (m, 4H), 0.69 (s, 3H), 0.66 (s, 3H), 0.64-0.56 (m, 1H), 0.37 (s, 6H).

### Synthesis of A-7

To a solution of A-6.9a (110 mg, 0.1886 mmol) in THF (2 mL) was added tetra-n-butylammonium fluoride (1 mL, 1M, 1 mmol) in one portion at 50° C. After stirring at 50° C. for 16 hours the mixture was poured into water (10 mL) and EtOAc (10 mL). The aqueous phase was extracted with EtOAc (2×10 mL) and the combined organic phases were dried over anhydrous Na₂SO₄, filtered, and concentrated. The residue was purified by silica gel chromatography (0˜30% EtOAc in PE) to give A-7 (66.4 mg, 79%). ¹H NMR (400 MHz, CDCl₃) δ_{H} 3.32 (s, 3H), 3.29-3.23 (m, 2H), 2.71 (d, J 3.6 Hz, 1H), 2.02-1.96 (m, 1H), 1.91-1.81 (m, 1H), 1.71-1.58 (m, 8H), 1.56-1.32 (m, 9H), 1.28-0.96 (m, 11H), 0.94-0.91 (m, 6H), 0.88 (t, J 7.6 Hz, 3H), 0.83 (s, 3H), 0.71-0.58 (m, 4H). LC-ELSD/MS purity >99%, MS ESI calcd For C₂₉H₅₂O₃ [M+H-CH₃OH]⁺ 417.4, found 417.4.

### Synthesis of A-6

To a solution of A-6.9b (140 mg, 0.2401 mmol) in THF (2 mL) was added tetra-n-butylammonium fluoride (1 mL, 1M, 1 mmol) in one portion at 50° C. After stirring at 50° C. for 16 hours the mixture was poured into water (10 mL) and EtOAc (10 mL). The aqueous phase was extracted with EtOAc (2×10 mL), and the organics were combined, dried over anhydrous Na₂SO₄, filtered, and concentrated. The residue was purified by silica gel chromatography (0˜30% EtOAc in PE) to give A-6 (88.9 mg, 83%). ¹H NMR (400 MHz, CDCl₃) δ_{H} 3.37-3.20 (m, 5H), 2.06-1.96 (m, 1H), 1.75-1.58 (m, 10H), 1.56-1.33 (m, 9H), 1.30-0.97 (m, 11H), 0.95-0.90 (m, 6H), 0.87 (t, J 7.6 Hz, 3H), 0.83 (s, 3H), 0.70-0.62 (m, 4H). LC-ELSD/MS purity >99%, MS ESI calcd. For C₂₉H₅₂O₃ [M+H−CH₃OH]⁺ 417.4, found 417.4.

### Example 6: Synthesis of 2-((3S,5S,8R,9S,10S,13S,14S,17R)-3-ethyl-3-hydroxy-10,13-dimethylhexadecahydro-1H-cyclopenta[a]phenanthren-17-yl)-6-hydroxy-6-methylheptanenitrile (A-8) and (R)-2-((3S,5S,8R,9S,10S,13S,14S,17R)-3-ethyl-3-hydroxy-10,13-dimethylhexadecahydro-1H-cyclopenta[a]phenanthren-17-yl)-6-hydroxy-6-methylheptanenitrile (A-9)

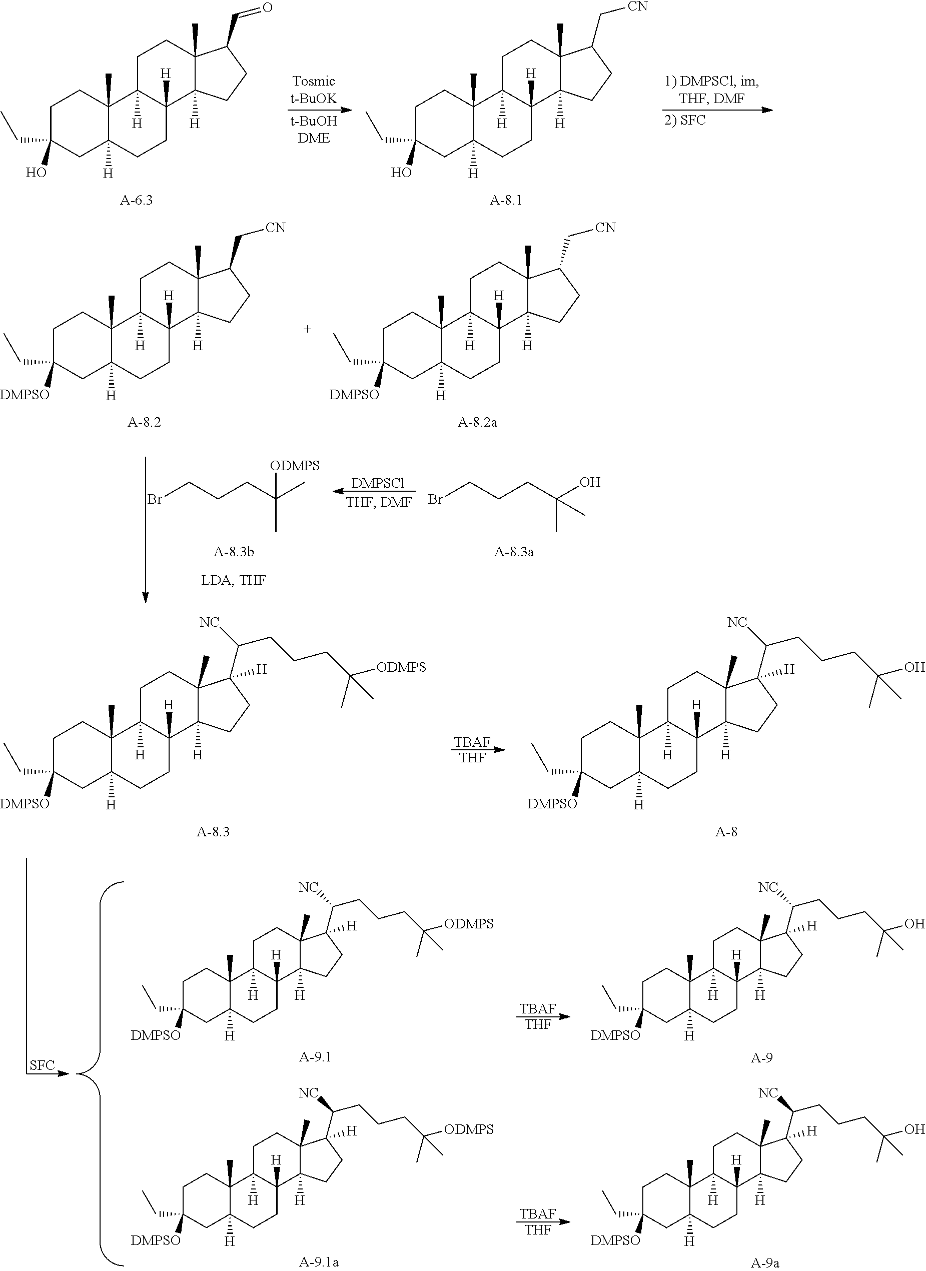

### Synthesis of A-8.1

To a suspension of A-6.3 (2.20 g, 6.61 mmol) in t-BuOH (40 mL) and DME (40 mL) was added t-BuOK (3.70 g, 33.0 mmol). After stirring at 20° C. for 30 minutes, Tosmic (2.57 g, 13.2 mmol) was added and the mixture was stirred at 20° C. for 16 hours. The mixture was quenched with 10% NH₄Cl (50 mL) and extracted with EtOAc (2×50 mL). The combined organic phase was washed with H₂O (50 mL), dried over anhydrous Na₂SO₄, filtered, and concentrated and purified by flash column chromatography (0-30% of EtOAc in PE) to give A-8.1 (1.00 g, 44%).

### Synthesis of A-8.2 and A-8.2a

To a solution of A-8.1 (1.10 g, 3.20 mol) in THF/DMF (10 mL, 1:1) was added 1H-imidazole (326 mg, 4.80 mmol), DIPEA (1.23 g, 9.60 mmol) and chlorodimethylphenylsilane (1.09 g, 6.40 mmol). The reaction mixture was stirred at 25° C. for 16 hours. The mixture was then poured into aqueous saturated NaHCO₃ (20 mL), then extracted with EtOAc (3×20 mL). The combined organic phase was washed with saturated brine (20 mL), dried over anhydrous Na₂SO₄, filtered, and concentrated. The residue was purified by flash column (0˜10% of EtOAc in PE) followed by further purification by SFC (Column: DAICEL CHIRALPAK IC (250 mm*30 mm, 10 um); Condition: 0.1% NH₃H₂O in EtOH; Begin B: 25%; End B: 25%; FlowRate (ml/min): 70; Injections: 370) to afford A-8.2a (280 mg, 20%) and A-8.2 (760 mg, 54%).

A-8.2a: ¹H NMR (400 MHz, CDCl₃) δ_{H} 7.66-7.57 (m, 2H), 7.41-7.29 (m, 3H), 2.37-2.25 (m, 1H), 2.19-1.90 (m, 3H), 1.77-1.57 (m, 6H), 1.54-0.99 (m, 14H), 0.98-0.89 (m, 2H), 0.86 (t, J=7.2 Hz, 3H), 0.79 (s, 3H), 0.69 (s, 3H), 0.64-0.56 (m, 1H), 0.38 (s, 6H).

A-8.2: ¹H NMR (400 MHz, CDCl₃) δ_{H} 7.64-7.56 (m, 2H), 7.41-7.30 (m, 3H), 2.35-2.25 (m, 1H), 2.21-2.12 (m, 1H), 2.05-1.94 (m, 1H), 1.84-1.77 (m, 1H), 1.75-1.56 (m, 7H), 1.49-0.89 (m, 15H), 0.88-0.80 (m, 5H), 0.69 (s, 3H), 0.68-0.62 (m, 1H), 0.60 (s, 3H), 0.38 (s, 6H).

### Synthesis of A-8.3b

To a solution of A-8.3a (5.00 g, 27.6 mol) in THF/DMF (40 mL, 1:1) was added 1H-imidazole (2.81 g, 41.4 mmol), DIPEA (10.6 g, 82.8 mmol) and chlorodimethylphenylsilane (7.06 g, 41.4 mmol). The reaction mixture was stirred at 25° C. for 16 hours. The mixture was poured into aq. NaHCO₃ (50 mL) and extracted with EtOAc (3×50 mL). The combined organic phase was washed with saturated brine (40 mL), dried over anhydrous Na₂SO₄, filtered, and concentrated. The residue was purified by flash column (0˜5% of EtOAc in PE) to give A-8.3b (4.7 g). ¹H NMR (400 MHz, CDCl₃) δ_{H} 7.60-7.53 (m, 2H), 7.41-7.34 (m, 3H), 3.52 (t, J=6.7 Hz, 1H), 3.39 (t, J=6.8 Hz, 1H), 2.01-1.84 (m, 2H), 1.61-1.47 (m, 2H), 1.22 (s, 6H), 0.38 (s, 6H).

### Synthesis of A-8.3

To a solution of DIPA (631 mg, 6.24 mmol) in THF (5 mL) under N₂ was added BuLi (2.08 mL, 2.5 M, 5.20 mmol) at −70° C. and the mixture was warmed to 0° C. for 0.5 hour then cooled to −70° C. A solution of A-8.2 (500 mg, 1.04 mmol) in THF (5 mL) was added dropwise under N₂ and stirred at −70° C. for 1 hour. Next, A-8.3b (983 mg, 3.12 mmol) was added and the mixture was stirred at −70° C. for 4 hours then poured into saturated NH₄Cl (30 mL) and extracted with EtOAc (3×30 mL). The combined organic layer was washed with brine (30 mL), dried over anhydrous Na₂SO₄, filtered, and concentrated. The residue was purified by flash column (0˜5% of EtOAc in PE) to give A-8.3 (360 mg).

### Synthesis of A-8

To a suspension of A-8.3 (120 mg, 0.17 mmol) in THF (2 mL) was added TBAF (1.01 mL, 1M, 1.01 mmol) and the mixture was stirred at 20° C. for 16 hours. The mixture was quenched with water (30 mL) and extracted with EtOAc (2×20 mL). The combined organic phase was washed with brine (20 mL), dried over Na₂SO₄, filtered, and concentrated. The residue was purified by flash column (0˜50% of EtOAc in PE) to give A-8 (22.5 mg, 19%, 20-alpha-CN: 20-beta-CN˜2:3). ¹H NMR (400 MHz, CDCl₃) δ_{H} 2.62-2.54 (m, 0.6H), 2.37-2.29 (m, 0.8H), 2.06-1.53 (m, 14.6H), 1.47-1.26 (m, 8H), 1.25-1.21 (m, 8H), 1.21-0.92 (m, 6H), 0.88 (t, J=7.2 Hz, 3H), 0.84 (s, 3H), 0.74-0.71 (m, 3H), 0.70-0.60 (m, 1H) LC-ELSD/MS 30-90AB_2 min_E, purity >99%, MS ESI calcd. for C₂₉H₄₉NO₂ [M−H₂O+H]⁺ 426.4, found 426.4.

### Synthesis of A-9.1 and A-9.1a

A-8.3 was separated by SFC (DAICEL CHIRALCEL OD-H (250 mm*30 mm, 5 um); Condition: 0.1% NH₃H₂O in EtOH; Begin B: 25%; End B: 25%; FlowRate (ml/min): 75; Injections: 120) to afford A-9.1a (120 mg, 33%) and A-9.1 (50.0 mg, 14%).

A-9.1a: ¹H NMR (400 MHz, CDCl₃) δ_{H} 7.65-7.54 (m, 4H), 7.41-7.30 (m, 6H), 2.59-2.46 (m, 1H), 2.05-1.85 (m, 1H), 1.77-1.56 (m, 8H), 1.52-1.23 (m, 12H), 1.20 (s, 6H), 1.19-0.88 (m, 9H), 0.85 (t, J=7.3 Hz, 3H), 0.68 (s, 6H), 0.64-0.57 (m, 1H), 0.37 (s, 12H).

A-9.1: ¹H NMR (400 MHz, CDCl₃) δ_{H} 7.71-7.49 (m, 4H), 7.42-7.29 (m, 6H), 2.33-2.21 (m, 2H), 1.89-1.51 (m, 11H), 1.50-1.24 (m, 9H), 1.21 (s, 6H), 1.19-0.88 (m, 9H), 0.86 (t, J=7.3 Hz, 3H), 0.70 (s, 3H), 0.69 (s, 3H), 0.67-0.60 (m, 1H), 0.37 (s, 12H).

### Synthesis of A-9

To a suspension of A-9.1 (55.0 mg, 0.08 mmol) in THF (1 mL) was added TBAF (1.15 mL, 1M, 1.15 mmol) and the mixture was stirred at 20° C. for 16 hours. The mixture was quenched with water (10 mL) and extracted with EtOAc (3×10 mL). The combined organic phase was washed with brine (10 mL), dried over Na₂SO₄, filtered, and concentrated. The residue was purified by flash column (0˜30% of EtOAc in PE) to give A-9 (33.4 mg, 61%). ¹H NMR (400 MHz, CDCl₃) δ_{H} 2.38-2.27 (m, 2H), 1.91-1.79 (m, 1H), 1.72-1.57 (m, 8H), 1.51-1.25 (m, 12H), 1.25-1.19 (m, 9H), 1.18-0.90 (m, 7H), 0.88 (t, J=7.6 Hz, 3H), 0.84 (s, 3H), 0.73 (s, 3H), 0.71-0.64 (m, 1H) LC-ELSD/MS 30-90AB_2 min_E, purity >99%, MS ESI calcd. for C₂₉H₄₉NO₂ [M−H₂O+H]⁺ 426.4, found 426.4.

### Synthesis of A-9a

To a suspension of A-9.1a (120 mg, 0.17 mmol) in THF (1 mL) was added TBAF (1.01 mL, 1M, 1.01 mmol). The mixture was stirred at 20° C. for 16 hours, then quenched with water (20 mL), and extracted with EtOAc (3×20 mL). The combined organic phase was washed with brine (20 mL), dried over Na₂SO₄, filtered, and concentrated. The residue was purified by flash column (0˜30% of EtOAc in PE) to give A-9a (34.6 mg, 29%). ¹H NMR (400 MHz, CDCl₃) δ_{H} 2.64-2.54 (m, 1H), 2.08-1.89 (m, 1H), 1.80-1.72 (m, 1H), 1.72-1.58 (m, 8H), 1.54-1.27 (m, 13H), 1.25-1.21 (m, 8H), 1.21-0.90 (m, 7H), 0.88 (t, J=7.6 Hz, 3H), 0.83 (s, 3H), 0.72 (s, 3H), 0.70-0.62 (m, 1H) LC-ELSD/MS 30-90AB_2 min_E, purity >99%, MS ESI calcd. for C₂₉H₄₉NO₂ [M−H₂O+H]⁺ 426.4, found 426.4.

### Example 7: Synthesis of (3S,5S,8R,9S,10S,13S,14S,17S)-3-ethyl-17-((R)-5-hydroxy-1-methoxy-5-methylhexyl)-10,13-dimethylhexadecahydro-1H-cyclopenta[a]phenanthren-3-ol (A-10) and (3S,8S,9S,10R,13S,14S,17S)-3-ethyl-17-((R)-5-hydroxy-1-methoxy-5-methylhexyl)-10,13-dimethyl-2,3,4,7,8,9,10,11,12,13,14,15,16,17-tetradecahydro-1H-cyclopenta[a]phenanthren-3-ol (A-12)

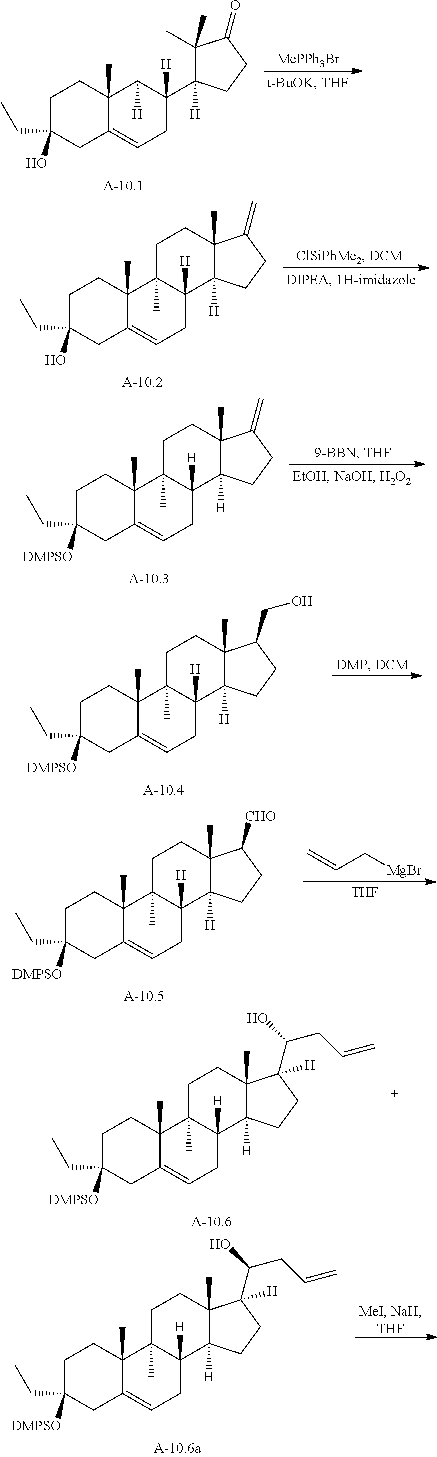

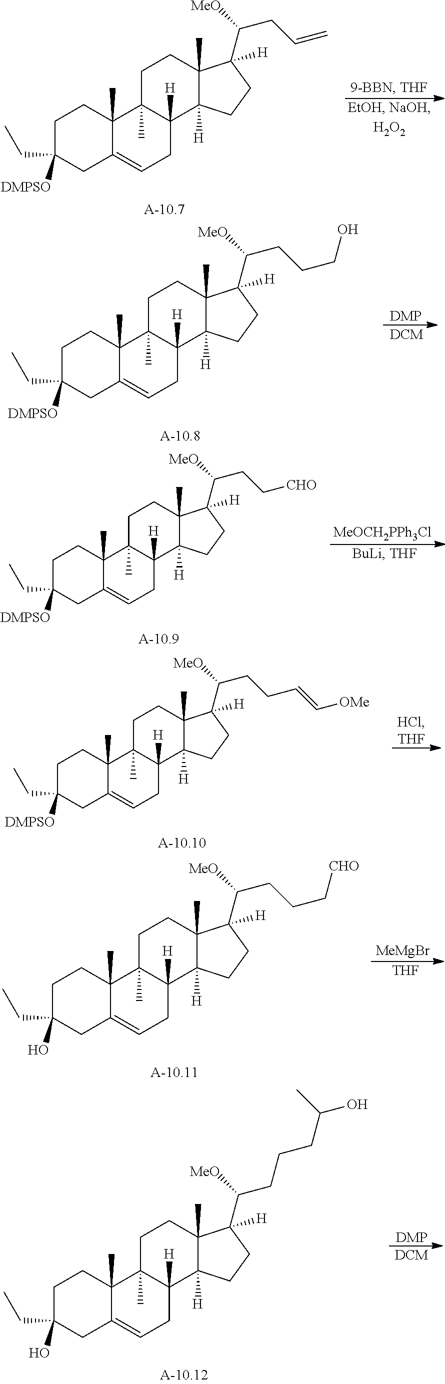

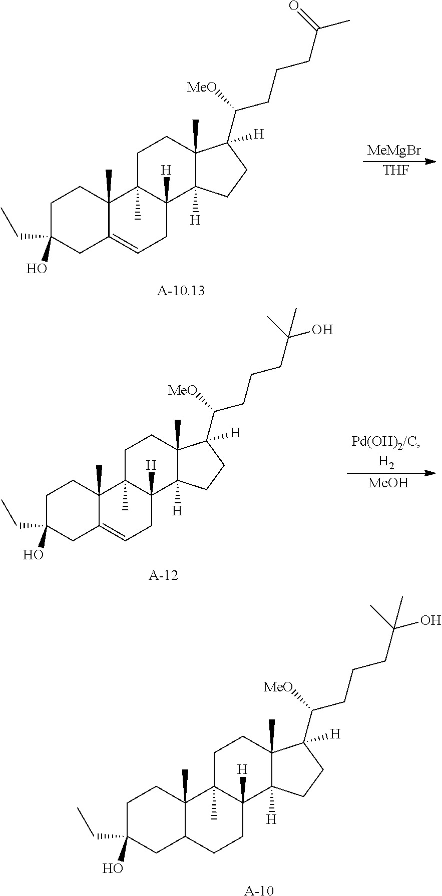

### Synthesis of A-10.2

To a solution of MePPh₃Br (168 g, 472 mmol) in THF (800 mL) was added t-BuOK (52.9 g, 472 mmol) at 20° C. under N₂. After stirring at 50° C. for 30 minutes A-10.1 (100 g, 315 mmol) was added and the mixture was maintained at 55° C. for 16 hours. The mixture was poured into saturated NH₄Cl (1000 mL) and extracted with EtOAc (3×500 mL). The combined organic layer was washed with brine (500 mL), dried over anhydrous Na₂SO₄, filtered, concentrated, and triturated with MeOH/H₂O (1200 mL/1200 mL) to give A-10.2 (118 g, 51.5%). ¹H NMR (400 MHz, CDCl₃) δ_{H} 5.30 (t, J=2.4 Hz, 1H), 4.70-4.55 (m, 2H), 2.59-2.45 (m, 1H), 2.43-2.32 (m, 1H), 2.30-2.18 (m, 1H), 2.14-1.96 (m, 2H), 1.89-1.24 (m, 15H), 1.06 (s, 3H), 1.02-0.91 (m, 2H), 0.85 (t, J=7.6 Hz, 3H), 0.80 (s, 3H).

### Synthesis of A-10.3

To a solution of A-10.2 (118 g, 375 mmol) in THF (500 mL) and DMF (500 mL) was added 1H-imidazole (38.2 g, 562 mmol), DIPEA (72.5 g, 562 mmol), and chlorodimethylphenylsilane (64.0 g, 375 mmol). The reaction mixture was stirred at 25° C. for 16 hours, the reaction was quenched with H₂O (500 mL), and the mixture was concentrated. The residue was extracted with PE (2×500 mL) and the combined organic phase was washed with brine (500 mL), dried over anhydrous Na₂SO₄, filtered, and concentrated to give A-10.3 (154 g, 91.6%). ¹H NMR (400 MHz, CDCl₃) δ_{H} 7.66-7.59 (m, 2H), 7.37-7.34 (m, 3H), 5.48-5.10 (m, 1H), 4.67-4.58 (m, 2H), 2.57-2.37 (m, 2H), 2.30-2.20 (m, 1H), 2.14-1.95 (m, 2H), 1.74-1.37 (m, 13H), 0.94 (s, 3H), 0.90-0.81 (m, 6H), 0.78 (s, 3H), 0.40 (s, 6H).

### Synthesis of A-10.4

To a solution of A-10.3 (144 g, 320 mmol) in anhydrous THF (1500 mL) was added 9-BBN dimer (78.0 g, 320 mmol) at 25° C. under N₂ and the reaction mixture was stirred at 25° C. for 16 hours. To the resulting mixture was added ethanol (112 mL, 1.92 mol) at 25° C. and NaOH (384 mL, 5.0 M, 1.92 mol) followed by dropwise addition of H₂O₂ (192 mL, 10 M, 1.92 mol) at 25° C. The reaction mixture was cooled to 20° C., poured into Na₂SO₃ (500 mL, saturated aq.), and extracted with EtOAc (3×500 mL). The combined organic phase was washed with saturated brine (500 mL), dried over anhydrous Na₂SO₄, filtered, and concentrated to give crude product. The crude product was triturated with MeOH/H₂O (1000 mL/1000 mL) to give A-10.4 (159 g). ¹H NMR (400 MHz, CDCl₃) δ_{H} 7.65-7.59 (m, 2H), 7.38-7.34 (m, 3H), 5.25-5.20 (m, 1H), 3.77-3.65 (m, 1H), 3.60-3.49 (m, 1H), 2.52-2.35 (m, 1H), 2.13-2.03 (m, 1H), 1.92-1.82 (m, 3H), 1.57-0.98 (m, 18H), 0.92 (s, 3H), 0.82 (t, J=7.2 Hz, 3H), 0.65 (s, 3H), 0.39 (s, 6H).

### Synthesis of A-10.5

To a solution of A-10.4 (55.0 g, 117 mmol) in DCM (550 mL) was added DMP (59.3 g, 140 mmol) at 25° C. and the mixture was stirred at 25° C. for 1 hour. The mixture was added to NaHCO₃ (550 mL, sat.) and Na₂S₂O₃ (550 mL, sat.) and then combined with another batch. The aqueous phase was extracted with DCM (2×500 mL) and the combined organic phase was washed with NaHCO₃/Na₂S₂O₃ (1:1, 500 mL, sat.), brine (500 mL), dried over anhydrous Na₂SO₄, filtered, and concentrated to give A-10.5 (85.0 g). ¹H NMR (400 MHz, CDCl₃) δ_{H} 9.77 (d, J=2.4 Hz, 1H), 7.66-7.57 (m, 2H), 7.37-7.34 (m, 3H), 5.26-5.20 (m, 1H), 2.45-2.36 (m, 1H), 2.33-2.24 (m, 1H), 2.15-1.97 (m, 4H), 1.80-1.60 (m, 7H), 1.49-1.33 (m, 6H), 1.14-0.99 (m, 3H), 0.92 (s, 3H), 0.83 (t, J=7.2 Hz, 3H), 0.75 (s, 3H), 0.39 (s, 6H).

### Synthesis of A-10.6 and A-10.6a

To a solution of allylmagnesium bromide (278 mL, 278 mmol, 1M) was slowly added a solution of A-10.5 (65.0 g, 139 mmol) in THF (300 mL) at 0° C. under N₂. After stirring at 25° C. for 2 hours, the mixture was poured into 10% NH₄Cl (800 mL) and extracted with EtOAc (2×500 mL). The combined organic phase was washed with 10% NH₄Cl (500 mL), dried over Na₂SO₄, filtered, and concentrated. Purification by flash column chromatography (0-5% of EtOAc in PE) provided A-10.6 (20.0 g, 20.0%) and A-10.6a (15.0 g, 17.7%).

A-10.6: ¹H NMR (400 MHz, CDCl₃) δ_{H} 7.65-7.59 (m, 2H), 7.37-7.31 (m, 3H), 5.93-5.73 (m, 1H), 5.27-5.19 (m, 1H), 5.16 (s, 1H), 5.15-5.11 (m, 1H), 3.68-3.46 (m, 1H), 2.47-2.28 (m, 2H), 2.05 (s, 4H), 1.74-1.60 (m, 5H), 1.55-0.97 (m, 15H), 0.93-0.90 (m, 3H), 0.83 (t, J=7.2 Hz, 3H), 0.76 (s, 2H), 0.39 (s, 6H).

A-10.6a: ¹H NMR (400 MHz, CDCl₃) δ_{H} 7.65-7.57 (m, 2H), 7.39-7.31 (m, 3H), 5.90-5.75 (m, 1H), 5.26-5.20 (m, 1H), 5.19-5.07 (m, 3H), 3.67-3.57 (m, 1H), 2.47-2.33 (m, 2H), 2.16-1.82 (m, 5H), 1.70-1.61 (m, 5H), 1.52-1.34 (m, 8H), 1.17-0.96 (m, 4H), 0.91 (s, 3H), 0.83 (t, J=7.2 Hz, 3H), 0.70 (s, 3H), 0.39 (s, 6H).

### Synthesis of A-10.7

To a suspension of NaH (7.83 g, 60%, 196 mmol) in THF (50 mL) was added HMPA (7.06 g, 39.4 mmol) and A-10.6 (20.0 g, 39.4 mmol) in THF (100 mL) at 0° C. The mixture was stirred at 25° C. for 30 minutes then CH₃I (12.1 mL, 196 mmol) was added at 25° C. The mixture was stirred at 25° C. for 16 hours and then the reaction was quenched with saturated NH₄Cl (100 mL). The mixture was extracted with EtOAc (2×200 mL). The combined organic phase was washed with brine (200 mL), dried over anhydrous Na₂SO₄, filtered, and concentrated to give A-10.7 (27.0 g). ¹H NMR (400 MHz, CDCl₃) δ_{H} 7.65-7.57 (m, 2H), 7.38-7.32 (m, 3H), 5.98-5.79 (m, 1H), 5.25-5.19 (m, 1H), 5.11-5.03 (m, 2H), 3.32 (s, 3H), 2.56-2.35 (m, 2H), 2.12-1.98 (m, 3H), 1.73-1.36 (m, 13H), 1.31-1.20 (m, 6H), 0.92 (s, 3H), 0.85-0.80 (m, 4H), 0.68 (s, 3H), 0.39 (s, 6H).

### Synthesis of A-10.8

To a solution of A-10.7 (26.0 g, 49.9 mmol) in anhydrous THF (300 mL) was added 9-BBN dimer (24.3 g, 99.8 mmol) at 25° C. under N₂ and the reaction mixture was stirred at 25° C. for 16 hours. To the resulting mixture was added ethanol (29.0 mL, 499 mmol) at 25° C., followed by NaOH (99.8 mL, 5.0 M, 499 mmol) and dropwise addition of H₂O₂ (49.9 mL, 10 M, 499 mmol) at 25° C. The reaction mixture was then cooled to 20° C., poured into Na₂SO₃ (200 mL, sat. aq.) and extracted with EtOAc (3×300 mL). The combined organic phase was washed with brine (500 mL), dried over anhydrous Na₂SO₄, filtered, and concentrated to give the product. The product was triturated with MeOH/H₂O (300 mL/300 mL) and purified by flash column chromatography (0-20% EtOAc in PE) to give A-10.8 (12.0 g, 48.1%). 1H NMR (400 MHz, CDCl₃) δ_{H} 7.65-7.57 (m, 2H), 7.43-7.29 (m, 3H), 5.32-5.14 (m, 1H), 3.70-3.51 (m, 2H), 3.32 (s, 3H), 3.28-3.21 (m, 1H), 2.46-2.35 (m, 1H), 2.10-1.80 (m, 4H), 1.75-1.33 (m, 17H), 1.20-0.97 (m, 5H), 0.92 (s, 3H), 0.82 (t, J=7.2 Hz, 3H), 0.68 (s, 3H), 0.39 (s, 6H).

### Synthesis of A-10.9

To a solution of A-10.8 (12.0 g, 23.2 mmol) in DCM (150 mL) was added DMP (18.8 g, 44.4 mmol) and the mixture was stirred at 25° C. for 1 hour. The mixture was added to NaHCO₃ (100 mL, sat.) and Na₂S₂O₃ (100 mL, sat.) then extracted with DCM (2×100 mL). The combined organic phase was washed with NaHCO₃/Na₂S₂O₃ (1:1, 100 mL, sat.), brine (100 mL), dried over anhydrous Na₂SO₄, filtered, and concentrated to give A-10.9 (11.0 g, 92.4%). ¹H NMR (400 MHz, CDCl₃) δ_{H} 9.80 (t, J=1.6 Hz, 1H), 7.65-7.58 (m, 2H), 7.38-7.32 (m, 3H), 5.26-5.16 (m, 1H), 3.26 (s, 3H), 2.60-2.36 (m, 3H), 2.14-1.92 (m, 4H), 1.74-1.36 (m, 14H), 1.23-0.93 (m, 6H), 0.92 (s, 3H), 0.82 (t, J=7.2 Hz, 3H), 0.69 (s, 3H), 0.39 (s, 6H).

### Synthesis of A-10.10

To a solution of MeOCH₂PPh₃Cl (1.91 g, 5.58 mmol) in THF (5 mL) was added n-BuLi (1.86 mL, 2.5 M in THF, 4.65 mmol) at 0° C. The mixture was stirred at 0° C. for 0.5 hour and then A-10.9 (1.00 g, 1.86 mmol) in THF (10 mL) was added at 0° C. The mixture was stirred at 20° C. for 3 hours. The mixture was quenched by saturated NH₄Cl (10 mL) and extracted with DCM (2×20 mL). The combined organic phase was washed with brine (2×20 mL), dried over Na₂SO₄, filtered, and concentrated. The residue was purified by flash column (0˜20% of EtOAc in PE) to give A-10.10 (500 mg, 47.6%).

### Synthesis of A-10.11

To a solution of A-10.10 (450 mg, 0.7965 mmol) in THF (10 ml) was added in HCl (1 ml, 1 mmol) at 25° C. and the mixture was stirred at 25° C. for 16 hours. Saturated NaHCO₃ (10 mL) was added and the mixture was extracted with EtOAc (2×30 mL). The combined organic layers were washed with brine (30 mL), dried over Na₂SO₄, filtered, and concentrated. Purification by flash column chromatography (0-30% EtOAc in PE) provided A-10.11 (330 mg, 99.6%). ¹H NMR (400 MHz, CDCl₃) δ_{H} 9.76 (s, 1H), 5.31-5.25 (m, 1H), 3.29 (s, 3H), 2.48-2.32 (m, 3H), 2.12-1.92 (m, 4H), 1.76-1.60 (m, 9H), 1.53-1.32 (m, 8H), 1.20-1.08 (m, 4H), 1.04 (s, 4H), 1.00-0.96 (m, 1H), 0.85 (t, J=7.6 Hz, 3H), 0.70 (s, 3H).

### Synthesis of A-10.12

A solution of MeMgBr (0.53 mL, 3 M, 1.58 mmol) in THF (3 mL) was reacted with A-10.11 (330 mg, 0.792 mmol) in THF (5 mL) at 0° C. and the mixture was stirred at 25° C. for 2 hours. Saturated NH₄Cl (10 mL) was added and the mixture was extracted with EtOAc (2×30 mL). The combined organic layers were washed with brine (30 mL), dried over Na₂SO₄, filtered, and concentrated. Purification by flash column chromatography (0-40% EtOAc in PE) provided A-10.12 (130 mg, 38.0%). ¹H NMR (400 MHz, CDCl₃) δ_{H} 5.31-5.25 (m, 1H), 3.85-3.76 (m, 1H), 3.29 (s, 3H), 3.25-3.19 (m, 1H), 2.40-2.32 (m, 1H), 2.09-1.92 (m, 3H), 1.77-1.70 (m, 1H), 1.67-1.54 (m, 8H), 1.47-1.25 (m, 11H), 1.25-1.18 (m, 4H), 1.18-1.09 (m, 3H), 1.09-0.99 (m, 4H), 0.98-0.92 (m, 1H), 0.85 (t, J=7.2 Hz, 3H), 0.70 (s, 3H).

### Synthesis of A-10.13

To a solution of A-10.12 (110 mg, 0.2542 mmol) in DCM (2 ml) was added DMP (161 mg, 381 μmol) and the mixture was stirred at 25° C. for 2 hours. The reaction was quenched with saturated NaHCO₃ (10 mL) and Na₂S₂O₃ (10 ml), followed by extraction with DCM (2×30 mL). The combined organic layers were washed with NaHCO₃/Na₂S₂O₃ aqueous (1:1, 20 ml), brine (30 mL), dried over Na₂SO₄, filtered, and concentrated. Purification by flash column chromatography (0-40% EtOAc in PE) provided A-10.13 (76.0 mg, 69.7%). ¹H NMR (400 MHz, CDCl₃) δ_{H} 5.28 (d, J=4.8 Hz, 1H), 3.29 (s, 3H), 3.26-3.18 (m, 1H), 2.46-2.34 (m, 3H), 2.14 (s, 3H), 2.09-1.89 (m, 4H), 1.76-1.59 (m, 10H), 1.49-1.36 (m, 6H), 1.21-1.07 (m, 5H), 1.04 (s, 3H), 0.97-0.92 (m, 1H), 0.85 (t, J=7.2 Hz, 3H), 0.69 (s, 3H). LC-ELSD/MS 30-90AB_2 min_E, purity=96.5%; MS ESI calcd for C₂₈H₄₆O₃ [M−H₂O−MeOH+H]⁺ 381.3, found 381.3.

### Synthesis of A-12

A solution of MeMgBr (0.1 mL, 3 M, 0.3 mmol) in THF (0.2 mL) was reacted with A-10.13 (65.0 mg, 0.15 mmol) in THF (2 mL) at 0° C. The mixture was stirred at 25° C. for 2 hours. Saturated NH₄Cl (10 mL) was added and the mixture was extracted with EtOAc (2×30 mL). The combined organic layers were washed with brine (30 mL), dried over Na₂SO₄, filtered, and concentrated. Purification by flash column chromatography (0-40% EtOAc in PE) provided A-12 (65.0 mg, 97%). ¹H NMR (400 MHz, CDCl₃) δ_{H} 5.28 (d, J=5.2 Hz, 1H), 3.30 (s, 3H), 3.26-3.20 (m, 1H), 2.40-2.32 (m, 1H), 2.10-1.94 (m, 3H), 1.77-1.70 (m, 1H), 1.67-1.57 (m, 7H), 1.50-1.32 (m, 12H), 1.22 (s, 6H), 1.19-1.07 (m, 4H), 1.04 (s, 3H), 1.01-0.90 (m, 2H), 0.85 (t, J=7.6 Hz, 3H), 0.70 (s, 3H). LC-ELSD/MS 30-90AB_2 min_E, purity >99%; MS ESI calcd for C₂₉H₅₀O₃ [M−2H₂O-MeOH+H]⁺ 379.3, found 379.3.

### Synthesis of A-10

To a solution of A-12 (60.0 mg, 134 umol) in MeOH (1 mL) was added wet Pd(OH)₂/C (20.0 mg) at 20° C. The suspension was degassed under vacuum and purged with H₂ three times and then stirred under H₂ (50 psi) at 50° C. for 16 hours. The reaction mixture was filtered through a pad of Celite and washed with MeOH (3×10 mL). The filtrate was concentrated, purified by flash column (0˜40% of EtOAc in PE), and further lyophilized to give A-10 (4.20 mg, 6.97%). ¹H NMR (400 MHz, CDCl₃) δ_{H} 3.28 (s, 3H), 3.24-3.19 (m, 1H), 2.04-1.98 (m, 1H), 1.65-1.60 (m, 4H), 1.49-1.24 (m, 17H), 1.22 (s, 6H), 1.16-0.92 (m, 8H), 0.87 (t, J=7.6 Hz, 5H), 0.83 (s, 3H), 0.70-0.55 (m, 4H). LC-ELSD/MS 30-90AB_2 min_E, purity >99%; MS ESI calcd for C₂₉H₅₂O₃ [M−2H₂O−MeOH+H]⁺ 381.3, found 381.3.

### Example 8: Synthesis of (3S,5S,8R,9S,10S,13S,14S,17S)-17-((1R,4S)-1-ethoxy-4-hydroxy-5-methylhexyl)-3-ethyl-10,13-dimethylhexadecahydro-1H-cyclopenta[a]phenanthren-3-ol (A-11) and (3S,5R,8R,9S,10S,13S,14S,17S)-17-((1R,4S)-1-ethoxy-4-hydroxy-5-methylhexyl)-3-ethyl-10,13-dimethylhexadecahydro-1H-cyclopenta[a]phenanthren-3-ol (A-11a)

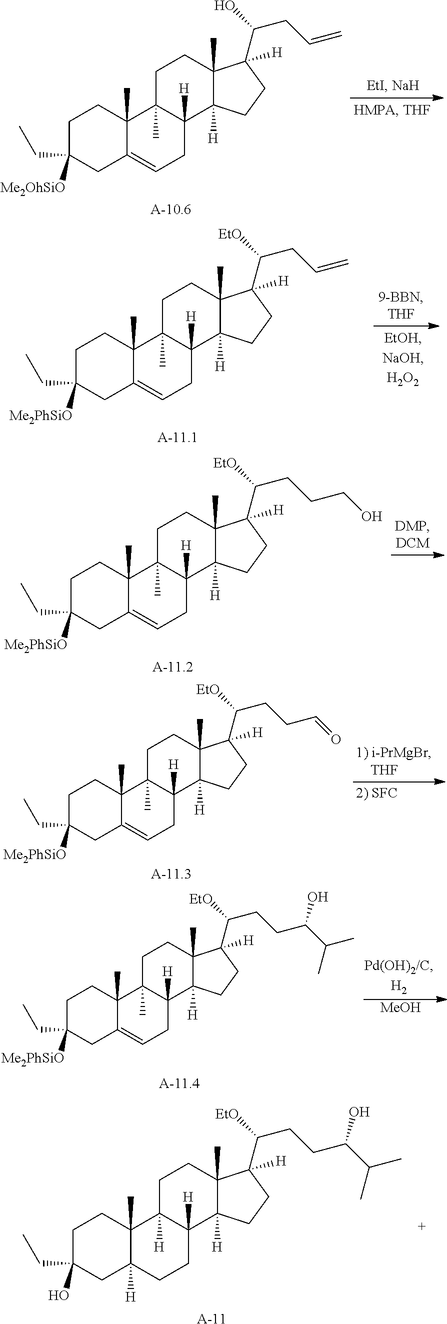

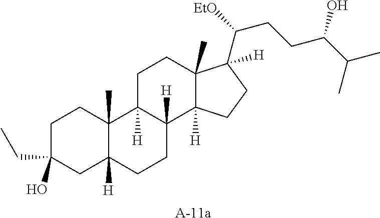

### Synthesis of A-11.1

To a solution of A-10.6 (2.00 g, 3.94 mmol) in THF (20 ml) was added NaH (2.35 g, 59.1 mmol, 60%) at 0° C. The mixture was stirred at 20° C. for 30 minutes and then iodoethane (9.21 g, 59.1 mmol) was added followed by HMPA (1.41 g, 7.88 mmol). The mixture was stirred at 40° C. for 16 hours then poured into 10% NH₄Cl (30 mL) and extracted with EtOAc (2×20 mL). The combined organic phase was washed with 10% NH₄Cl (30 mL), dried over Na₂SO₄, filtered, and concentrated. The residue was purified by flash column (0-10% of EtOAc in PE) to give A-11.1 (1.60 g, 76.1%). ¹H NMR (400 MHz, CDCl₃) δ_{H} δ 7.74-7.53 (m, 2H), 7.42-7.29 (m, 3H), 5.99-5.82 (m, 1H), 5.30-5.16 (m, 1H), 5.11-5.01 (m, 2H), 3.74-3.53 (m, 1H), 3.46-3.22 (m, 2H), 2.56-2.34 (m, 2H), 2.12-2.02 (m, 3H), 1.99-1.87 (m, 1H), 1.72-1.34 (m, 13H), 1.21-1.15 (m, 4H), 1.14-0.94 (m, 4H), 0.92 (s, 3H), 0.82 (t, J=6.8 Hz, 3H), 0.69 (s, 3H), 0.39 (s, 6H)

### Synthesis of A-11.2

To a solution of A-11.1 (1.60 g, 2.99 mmol) in anhydrous THF (15 mL) was added 9-BBN dimer (1.45 g, 5.98 mmol) at 25° C. under N₂. The reaction mixture was stirred at 25° C. for 16 hours. To the resulting mixture was added ethanol (1.64 g, 35.8 mmol) at 25° C., followed by aqueous NaOH (1.43 g, 5 M, 35.8 mmol) and dropwise addition of H₂O₂ (3.57 mL, 10 M, 35.8 mmol) at 60° C. The reaction mixture was cooled to 20° C. and poured into saturated Na₂SO₃ (30 mL) and extracted with EtOAc (3×30 mL). The combined organic phase was washed with saturated brine (30 mL), dried over anhydrous Na₂SO₄, filtered, and concentrated to give product. The product was purified by flash column (0-10% of EtOAc in PE) to give A-11.2 (1.10 g, 66.6%). ¹H NMR (400 MHz, CDCl₃) δ_{H} δ 7.66-7.56 (m, 2H), 7.41-7.29 (m, 3H), 5.26-5.18 (m, 1H), 3.72-3.49 (m, 3H), 3.42-3.28 (m, 2H), 2.45-2.37 (m, 1H), 2.30-2.15 (m, 1H), 1.96-1.89 (m, 1H), 1.84-1.55 (m, 12H), 1.54-1.29 (m, 10H), 1.20 (t, J=7.2 Hz, 3H), 1.17-0.94 (m, 6H), 0.92 (s, 3H), 0.82 (t, J=7.4 Hz, 3H), 0.68 (s, 3H), 0.39 (s, 6H).

### Synthesis of A-11.3

To a solution of A-11.2 (1.10 g, 1.98 mmol) in DCM (10 mL) was added DMP (1.67 g, 3.96 mmol) and the mixture was stirred at 25° C. for 1 hour. The reaction was quenched with NaHCO₃ (10 mL, sat.) and Na₂S₂O₃ (10 mL, sat.), and the mixture was extracted with DCM (2×10 mL). The combined organic phase was washed with NaHCO₃/Na₂S₂O₃ (1:1, 10 mL, sat.), brine (10 mL), dried over anhydrous Na₂SO₄, filtered, and concentrated. Purification by flash column (0˜10% of EtOAc in PE) provided A-11.3 (518 mg, 47.5%). ¹H NMR (400 MHz, CDCl₃) δ_{H} 9.89-9.74 (m, 1H), 7.71-7.52 (m, 2H), 7.47-7.29 (m, 3H), 5.25-5.19 (m, 1H), 3.57-3.48 (m, 1H), 3.41-3.28 (m, 2H), 2.60-2.50 (m, 1H), 2.48-2.37 (m, 3H), 1.98-1.83 (m, 2H), 1.72-1.59 (m, 6H), 1.54-1.32 (m, 8H), 1.29-1.11 (m, 6H), 1.09-0.95 (m, 3H), 0.93-0.90 (m, 3H), 0.82 (s, 3H), 0.69 (s, 3H), 0.44-0.36 (m, 6H).

### Synthesis of A-11.4

A mixture of i-PrMgBr (1.41 mL, 2M, 2.82 mmol) in THF (5 mL) was reacted with A-11.3 (518 mg, 0.94 mmol) in THF (5 mL) at 0° C. The mixture was stirred for 1 hour and then quenched with saturated NH₄Cl (25 mL) at 0° C. The aqueous phase was extracted with DCM (3×50 mL) and the combined organic layers were dried over Na₂SO₄, filtered, and concentrated to give residue. The residue was purified by a silica gel column (EtOAc in PE, 0%˜20%) and further purified by SFC ((DAICEL CHIRALPAK AD (250 mm*30 mm, 10 um)); Condition: 0.1% NH₃H₂O in IPA, Begin B: 25%, End B: 25%, Flow rate (ml/min): 60, Injections: 60) to give A-11.4 (67.0 mg). ¹H NMR (400 MHz, CDCl₃) δ_{H} δ 7.71-7.55 (m, 2H), 7.41-7.30 (m, 3H), 5.33-5.10 (m, 1H), 3.66-3.51 (m, 1H), 3.44-3.23 (m, 3H), 2.48-2.34 (m, 1H), 1.98-1.88 (m, 1H), 1.77-1.51 (m, 17H), 1.48-1.31 (m, 7H), 1.28-1.22 (m, 1H), 1.10-0.98 (m, 3H), 0.82 (t, J=7.2 Hz, 3H), 0.82 (s, 3H), 0.68 (s, 3H), 0.39 (s, 6H)

### Synthesis of A-11 and A-11a

To a solution of A-11.4 (90.0 mg, 0.1512 mmol) in MeOH (2 mL) was added wet Pd(OH)₂/C (50.0 mg) at 20° C. The suspension was degassed under vacuum and purged with H₂ three times and stirred under H₂ (50 psi) at 50° C. for 16 hours. The reaction mixture was filtered, concentrated, and purified by flash column (0-10% of EtOAc in PE). Further lyophilization provided A-11 (6.60 mg, 14%) and A-11a (4.10 mg, 9%).

A-11: ¹H NMR (400 MHz, CDCl₃) δ_{H} 3.66 (dd, J=6.8, 8.8 Hz, 1H), 3.44-3.16 (m, 3H), 2.07-1.98 (m, 1H), 1.89-1.81 (m, 1H), 1.68-1.64 (m, 3H), 1.53-1.28 (m, 13H), 1.26-0.97 (m, 15H), 0.96-0.84 (m, 11H), 0.83 (s, 3H), 0.67 (s, 3H). LC-ELSD/MS purity 99.4%, MS ESI calcd for C₃₀H₅₄O₃ [M−EtOH+H]⁺ 417.3 found 417.3.

A-11a: ¹H NMR (400 MHz, CDCl₃) δ_{H} 3.66 (dd, J=7.2, 8.8 Hz, 1H), 3.39-3.32 (m, 2H), 3.27-3.20 (m, 1H), 2.10-2.00 (m, 1H), 1.94-1.65 (m, 6H), 1.50-1.23 (m, 16H), 1.23-1.15 (m, 5H), 1.15-1.04 (m, 5H), 0.96 (s, 3H), 0.94-0.89 (m, 9H), 0.67 (s, 3H). LC-ELSD/MS purity 98.9%, MS ESI calcd for C₃₀H₅₄O₃ [M−EtOH+H]⁺ 417.3 found 417.3.

### Example 9: Synthesis of (3S,8S,9S,10R,13S,14S,17S)-3-ethyl-10,13-dimethyl-17-((1R,4R)-5,5,5-trifluoro-4-hydroxy-1-methoxy-4-methylpentyl)-2,3,4,7,8,9,10,11,12,13,14,15,16,17-tetradecahydro-1H-cyclopenta[a]phenanthren-3-ol (A-13) and (3S,8S,9S,10R,13S,14S,17S)-3-ethyl-10,13-dimethyl-17-((1R,4S)-5,5,5-trifluoro-4-hydroxy-1-methoxy-4-methylpentyl)-2,3,4,7,8,9,10,11,12,13,14,15,16,17-tetradecahydro-1H-cyclopenta[a]phenanthren-3-ol (A-14)

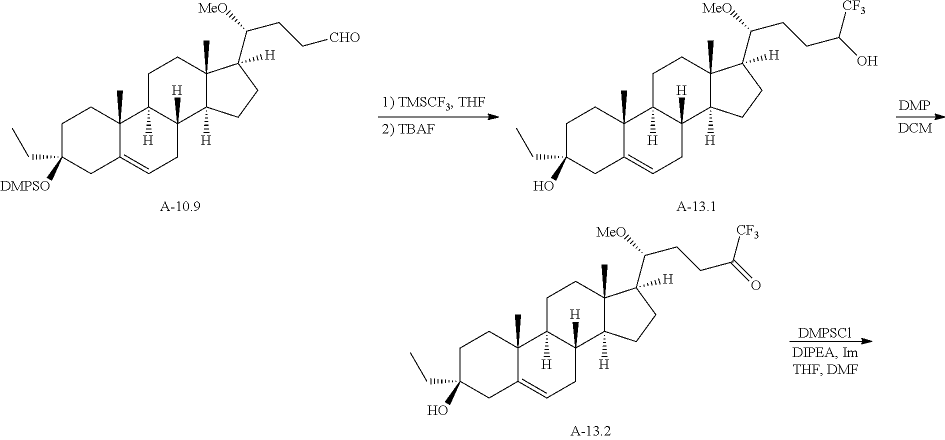

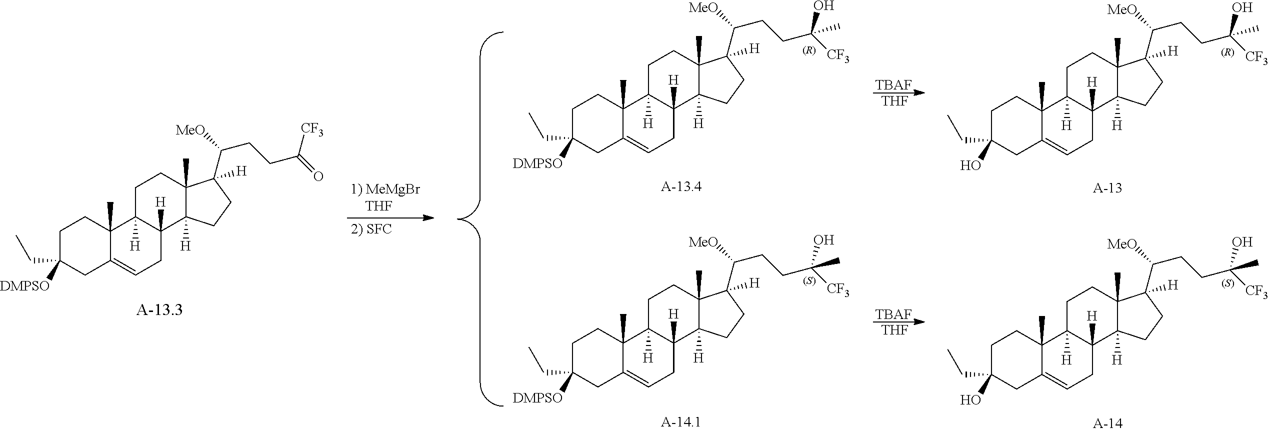

### Synthesis of A-13.1

To a solution of A-10.9 (2 g, 3.72 mmol) and CsF (1.13 g, 7.44 mmol) in THF (20 mL) at 15° C. was added TMSCF₃ (1.57 g, 11.1 mmol) under N₂. The mixture was stirred for 3 hours, followed by addition of TBAF (18.6 mL, 18.6 mmol, 1M TBAF in THF) and stirring at 15° C. for an additional 16 hours. The mixture was concentrated under vacuum and the resultant residue was dissolved in EtOAc (30 mL) and washed with water (2×16 mL). The organic phase was washed with brine (16 mL), dried over Na₂SO₄, filtered, and concentrated to give product. The product was purified by column chromatography (SiO₂, PE/EtOAc=10/1) to provide A-13.1 (1.4 g, 80%).

### Synthesis of A-13.2

To a solution of A-13.1 (200 mg, 0.42 mmol) in DCM (3 mL) was added DMP (534 mg, 1.26 mmol) and the mixture was stirred at 25° C. for 50 minutes. The mixture was quenched by addition of saturated NaHCO₃/Na₂S₂O₃ (1:1, 2×20 mL). The DCM phase was separated and washed with brine (20 mL), dried over Na₂SO₄, filtered, and concentrated to give A-13.2 (250 mg). ¹H NMR (400 MHz, CDCl₃) δ_{H} 5.28 (d, J=5.2 Hz, 1H), 3.32-3.26 (m, 1H), 3.23 (s, 3H), 2.90-2.59 (m, 2H), 2.41-2.32 (m, 1H), 2.21-2.12 (m, 1H), 2.03-1.94 (m, 2H), 1.77-1.44 (m, 17H), 1.19-1.07 (m, 4H), 1.04 (s, 3H), 0.85 (t, J=7.6 Hz, 3H), 0.71 (s, 3H). ¹⁹F NMR (376 MHz, CDCl₃) δ_{F} −79.02 (s).

### Synthesis of A-13.3

To a solution of A-13.2 (250 mg, 0.531 mmol) in THF (0.5 mL) and DMF (0.5 mL) was added 1H-imidazole (180 mg, 2.65 mmol), DIPEA (342 mg, 2.65 mmol), and chlorodimethylphenylsilane (452 mg, 2.65 mmol). The mixture was stirred at 25° C. for 16 hours, followed by addition of H₂O (10 mL) and extraction with EtOAc (2×30 mL). The combined organic phase was washed with brine (30 mL), dried over anhydrous Na₂SO₄, filtered, and concentrated to give the product. The residue was purified by column chromatography (PE/EtOAc=50/1 to 10/1) to provide A-13.3 (260 mg, 81%). ¹H NMR (400 MHz, CDCl₃) δ_{H} 7.67-7.58 (m, 2H), 7.36-7.33 (m, 3H), 5.25-5.20 (m, 1H), 3.35-3.20 (m, 4H), 2.90-2.77 (m, 1H), 2.71-2.58 (m, 1H), 2.45-2.36 (m, 1H), 2.21-1.89 (m, 4H), 1.72-1.60 (m, 8H), 1.50-1.35 (m, 8H), 1.25 (s, 3H), 0.92 (s, 3H), 0.82 (t, J=7.2 Hz, 3H), 0.68 (s, 3H), 0.41 (s, 6H). ¹⁹F NMR (376 MHz, CDCl₃) δ_{F} −79.02 (s).

### Synthesis of A-13.4 and A-14.1

To a solution of A-13.3 (260 mg, 0.43 mmol) in THF (5 mL) was added methyl magnesium bromide (2 mL, 6 mmol, 3M in THF) and the mixture was stirred at 25° C. for 16 hours. The mixture was poured into saturated NH₄Cl (10 mL), stirred for 10 minutes, and the aqueous phase was extracted with EtOAc (2×50 mL). The organic phase was washed with brine, filtered, and concentrated. The residue was purified by flash column (0˜30% of EtOAc in PE) and further separated by SFC (Column: DAICEL CHIRALPAK AD (250 mm*30 mm, 10 um); Condition: 0.1% NH₃H₂O in IPA; Begin B: 15; End B: 15; Gradient Time (min): n/a; 100% B Hold Time (min): n/a) to give A-14.1 (50 mg, 21%) and A-13.4 (50 mg, 21%).

A-14.1: ¹H NMR (400 MHz, CDCl₃) δ_{H} 7.61 (dd, J=3.2, 6.4 Hz, 2H), 7.39-7.30 (m, 3H), 5.22 (d, J=5.2 Hz, 1H), 3.32 (s, 3H), 3.30-3.26 (m, 1H), 2.46-2.36 (m, 1H), 2.03-1.79 (m, 4H), 1.71-1.60 (m, 8H), 1.54-1.35 (m, 8H), 1.31 (s, 3H), 1.23-0.95 (m, 6H), 0.91 (s, 3H), 0.82 (t, J=7.2 Hz, 3H), 0.69 (s, 3H), 0.39 (s, 6H). ¹⁹F NMR (376 MHz, CDCl₃) δ_{F} −83.17 (s).

A-13.4: ¹H NMR (400 MHz, CDCl₃) δ_{H} 7.61 (dd, J=3.2, 6.4 Hz, 2H), 7.42-7.31 (m, 3H), 5.22 (d, J=5.2 Hz, 1H), 3.32 (s, 3H), 3.29-3.23 (m, 1H), 2.44-2.36 (m, 1H), 2.10-1.60 (m, 13H), 1.53-1.35 (m, 8H), 1.33 (s, 3H), 1.20-1.01 (m, 5H), 0.92 (s, 3H), 0.82 (t, J=7.2 Hz, 3H), 0.69 (s, 3H), 0.39 (s, 6H). ¹⁹F NMR (376 MHz, CDCl₃) δ_{F} −81.28 (s).

### Synthesis of A-13

To a suspension of A-13.4 (50 mg, 0.08 mmol) in THF (2 mL) was added TBAF (0.402 mL, 0.402 mmol) and the mixture was stirred at 25° C. for 16 hours. Saturated NH₄Cl (20 mL) was added and the mixture was extracted with EtOAc (2×50 mL). The combined organic phase was washed with water (3×50 mL), dried over Na₂SO₄, filtered, and concentrated. Purification by flash column (0˜35% of EtOAc in PE) provided A-13 (20.6 mg, 53%). ¹H NMR (400 MHz, CDCl₃) δ_{H} 5.36-5.18 (m, 1H), 3.33 (s, 3H), 3.30-3.23 (m, 1H), 2.40-2.32 (m, 1H), 2.08-1.60 (m, 13H), 1.55-1.41 (m, 10H), 1.34 (s, 3H), 1.21-1.07 (m, 4H), 1.04 (s, 3H), 0.85 (t, J=7.4 Hz, 3H), 0.71 (s, 3H). ¹⁹F NMR (400 MHz, CDCl₃) δ_{F} −81.23 (s) LC-ELSD/MS: purity >99%, MS ESI calcd. for C₂₈H₄₅F₃O₃ [M+H−H₂O]⁺ 469.3, found 469.3.

### Synthesis of A-14

To a suspension of A-14.1 (50 mg, 0.08 mmol) in THF (2 mL) was added TBAF (0.402 mL, 0.402 mmol) the mixture was stirred at 25° C. for 16 hours. Saturated NH₄Cl (20 mL) was added and the mixture was extracted with EtOAc (2×50 mL). The combined organic phase was washed with water (3×50 mL), dried over Na₂SO₄, filtered, and concentrated. Purification by flash column (0˜35% of EtOAc in PE) provided A-14 (25.7 mg, 66%). ¹H NMR (400 MHz, CDCl₃) δ_{H} 5.28 (d, J=5.2 Hz, 1H), 3.33 (s, 3H), 3.31-3.26 (m, 1H), 2.42-2.31 (m, 1H), 2.06-1.82 (m, 5H), 1.76-1.53 (m, 10H), 1.50-1.36 (m, 7H), 1.32 (s, 3H), 1.23-1.08 (m, 4H), 1.04 (s, 3H), 1.00-0.94 (m, 1H), 0.85 (t, J=7.2 Hz, 3H), 0.71 (s, 3H). ¹⁹F NMR (400 MHz, CDCl₃) δ_{F} −83.17 (s). LC-ELSD/MS: purity >99%, MS ESI calcd. for C28H₄₅F₃O₃ [M+H−H₂O]⁺ 469.3, found 469.3.

### Example 10: Synthesis of (3S,8S,9S,10R,13S,14S,17S)-3-ethyl-17-((1R,4R)-4-hydroxy-1-methoxy-5-methylhexyl)-10,13-dimethyl-2,3,4,7,8,9,10,11,12,13,14,15,16,17-tetradecahydro-1H-cyclopenta[a]phenanthren-3-ol (A-15) and (3S,8S,9S,10R,13S,14S,17S)-3-ethyl-17-((1R,4S)-4-hydroxy-1-methoxy-5-methylhexyl)-10,13-dimethyl-2,3,4,7,8,9,10,11,12,13,14,15,16,17-tetradecahydro-1H-cyclopenta[a]phenanthren-3-ol (A-16)

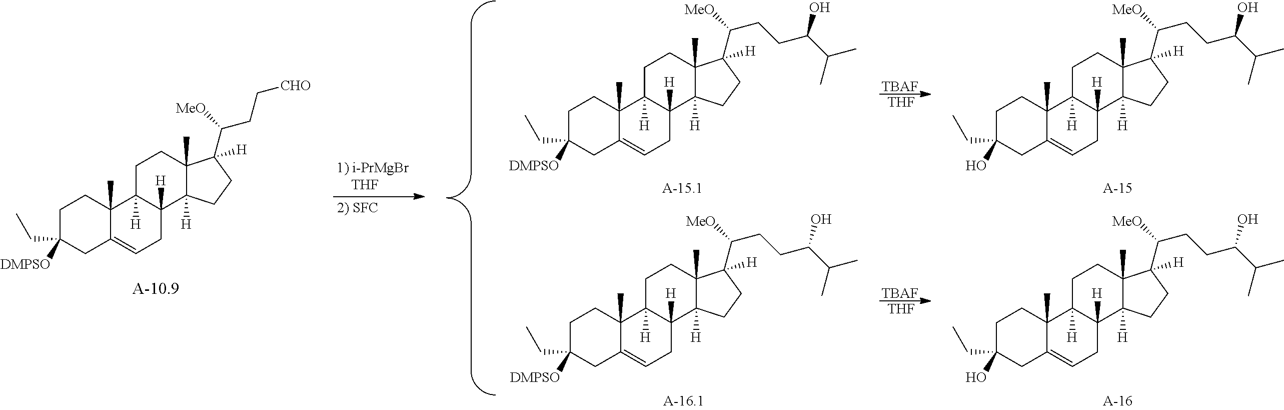

### Synthesis of A-15.1 and A-16.1

A mixture of i-PrMgBr (1.11 mL, 2M, 2.23 mmol) in THF (5 mL) was reacted with A-10.9 (400 mg, 0.75 mmol) in THF (5 mL) and the mixture was stirred at 0° C. for 1 hour. Saturated NH₄Cl (50 mL) was added at 0° C. and the mixture was extracted with EtOAc (3×50 mL). The combined organic layers were dried over Na₂SO₄ filtered, and concentrated. The residue was purified by a silica gel column and further separated by SFC (Column: DAICEL CHIRALPAK AD (250 mm*30 mm, 10 um); Condition: 0.1% NH₃H₂O in IPA; Begin B: 25%; End B: 25%; FlowRate (ml/min): 60; Injections: 60) to provide A-16.1 (90 mg) and A-15.1 (80 mg).

A-16.1: ¹H NMR (400 MHz, CDCl₃) δ_{H} 7.65-7.56 (m, 2H), 7.39-7.31 (m, 3H), 5.26-5.18 (m, 1H), 3.32 (s, 3H), 3.29-3.24 (m, 2H), 2.45-2.37 (m, 1H), 2.08-1.86 (m, 4H), 1.71-1.39 (m, 17H), 1.23-1.01 (m, 5H), 0.94-0.88 (m, 10H), 0.82 (t, J=7.2 Hz, 3H), 0.68 (s, 3H), 0.39 (s, 6H).

A-15.1: ¹H NMR (400 MHz, CDCl₃) δ_{H} 7.65-7.56 (m, 2H), 7.39-7.31 (m, 3H), 5.25-5.17 (m, 1H), 3.34-3.24 (m, 5H), 2.45-2.37 (m, 1H), 2.09-1.88 (m, 3H), 1.76-1.57 (m, 9H), 1.52-1.34 (m, 8H), 1.28-0.97 (m, 6H), 0.94-0.89 (m, 10H), 0.82 (t, J=7.6 Hz, 3H), 0.69 (s, 3H), 0.39 (s, 6H).

### Synthesis of A-15

To a suspension of A-15.1 (80 mg, 0.14 mmol) in THF (5 mL) was added TBAF (1 mL, 1 mmol) and the mixture was stirred at 20° C. for 16 hours. Saturated NH₄Cl (10 mL) was added and the mixture was extracted with EtOAc (2×20 mL). The combined organic phase was washed with water (50 mL), dried over Na₂SO₄, filtered, and concentrated. Purification by flash column (0˜30% of EtOAc in PE) followed by lyophilization provided A-15 (41.7 mg, 68%). ¹H NMR (400 MHz, CDCl₃) δ_{H} 5.31-5.25 (m, 1H), 3.36-3.23 (m, 5H), 2.40-2.32 (m, 1H), 2.10-1.91 (m, 3H), 1.76-1.54 (m, 12H), 1.49-1.07 (m, 11H), 1.05-0.95 (m, 5H), 0.95-0.85 (m, 6H), 0.85 (t, J=7.6 Hz, 3H), 0.71 (s, 3H). LC-ELSD/MS 30-90AB_2 min_E, purity >99%, MS ESI calcd. for C₂₉H₅₀₀₃ [M−CH₃OH+H]⁺ 415.4, found 415.4.

### Synthesis of A-16

To a suspension of A-16.1 (90 mg, 0.16 mmol) in THF (5 mL) was added TBAF (1 mL, 1 mmol) and the mixture was stirred at 20° C. for 16 hours. Saturated NH₄Cl (10 mL) was added and the mixture was extracted with EtOAc (2×20 mL). The combined organic phase was washed with water (50 mL), dried over Na₂SO₄, filtered, and concentrated. Purification by flash column (0˜30% of EtOAc in PE) followed by lyophilization provided A-16 (44.9 mg, 65%). ¹H NMR (400 MHz, CDCl₃) δ_{H} 5.32-5.25 (m, 1H), 3.33 (s, 3H), 3.30-3.24 (m, 2H), 2.40-2.32 (m, 1H), 2.08-1.66 (m, 8H), 1.58-1.36 (m, 16H), 1.20-1.07 (m, 4H), 1.04 (s, 3H), 0.94-0.91 (m, 6H), 0.85 (t, J=7.6 Hz, 3H), 0.70 (s, 3H). LC-ELSD/MS 30-90AB_2 min_E, purity >99%, MS ESI calcd. for C₂₉H₅₀O₃ [M−CH₃OH+H]⁺ 415.4, found 415.4.

### Example 11: Synthesis of (3S,5S,8R,9S,10S,13S,14S,17R)-3-ethyl-10,13-dimethyl-17-((2S,5S)-1,1,1-trifluoro-5-hydroxy-6-methylheptan-2-yl)hexadecahydro-1H-cyclopenta[a]phenanthren-3-ol (A-17)

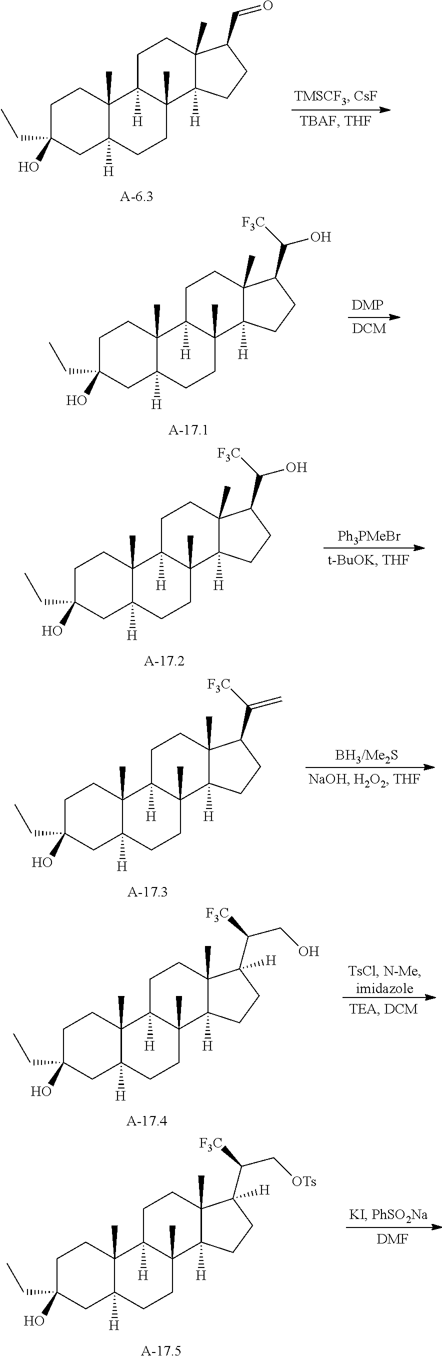

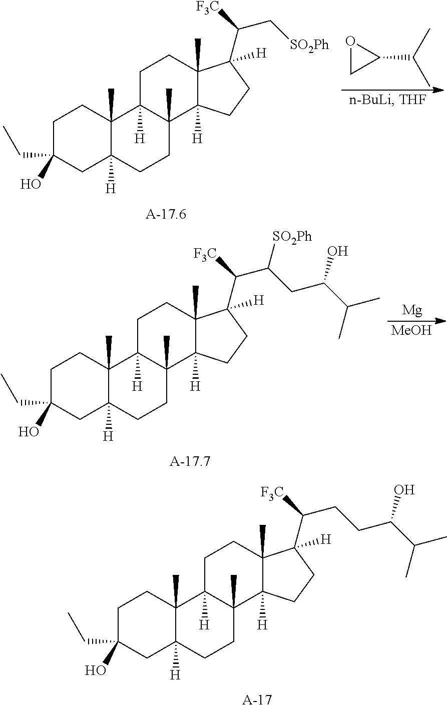

### Synthesis of A-17.1

To a solution of A-6.3 (23.6 g, 70.9 mmol) in THF (400 mL) was added CsF (2.14 g, 14.1 mmol) and TMSCF₃ (40.1 g, 283 mmol) and the mixture was stirred at 40° C. under N₂ for 1 hour. TBAF (89.1 g, 283 mmol) was added and the mixture was stirred at 50° C. for 16 hours and then poured into saturated NH₄Cl (300 mL). After stirring for 20 minutes the mixture was extracted with EtOAc (3×100 mL). The combined organic phase was washed with brine (150 mL), dried over Na₂SO₄, filtered, concentrated, and purified by flash column (0˜50% of EtOAc in PE) to give A-17.1 (22.0 g, 77.1%). ¹H NMR (400 MHz, CDCl₃) δ_{H} 4.06-3.84 (m, 1H), 1.81-1.64 (m, 6H), 1.56-1.46 (m, 3H), 1.44-1.30 (m, 6H), 1.19-0.93 (m, 8H), 0.91-0.81 (m, 9H), 0.78-0.69 (m, 4H).

### Synthesis of A-17.2

To a solution of A-17.1 (22.0 g, 54.6 mmol) in DCM (600 mL) was added DMP (46.2 g, 109 mmol). After stirring at 40° C. for 30 minutes, the mixture was quenched with aq. NaHCO₃ (200 mL, 2M) and saturated Na₂S₂O₃ (200 mL), and extracted with DCM (2×100 mL). The combined organic phase was washed with brine (200 mL), dried over Na₂SO₄, filtered, and concentrated. The residue was purified by flash column chromatography (PE/EtOAc=30/1 to 20/1) to afford A-17.2 (11.0 g, 50%). ¹H NMR (400 MHz, CDCl₃) δ_{H} 2.98-2.90 (m, 1H), 2.21-2.06 (m, 1H), 1.95-1.60 (m, 7H), 1.51-1.32 (m, 6H), 1.28-1.04 (m, 7H), 1.02-0.96 (m, 1H), 0.91-0.81 (m, 8H), 0.91-0.67 (m, 4H).

### Synthesis of A-17.3

To a suspension of Ph₃PMeBr (8.00 g, 22.4 mmol) in THF (40 mL) was added t-BuOK (2.51 g, 22.4 mmol) at 20° C. under N₂. After stirring at 20° C. for 30 minutes, a solution of A-17.2 (4.50 g, 11.2 mmol) in THF (40 mL) was added drop-wise. After stirring at 20° C. for another 2 hours under N₂, the reaction mixture was poured into saturated NH₄Cl (100 mL) and extracted with EtOAc (3×20 mL). The combined organic phase was washed with brine (100 mL), dried over anhydrous Na₂SO₄, filtered, and concentrated. The residue was purified by flash column chromatography (0˜20% of EtOAc in PE) to give A-17.3 (4.00 g, 90%). 1H NMR (400 MHz, CDCl₃) δ_{H} 5.86-5.83 (m, 1H), 5.45-5.40 (m, 1H), 2.34-2.24 (m, 1H), 1.96-1.85 (m, 1H), 1.74-1.66 (m, 4H), 1.61-1.52 (m, 6H), 1.45-1.35 (m, 4H), 1.17-1.09 (m, 3H), 1.03-0.92 (m, 2H), 0.91-0.87 (m, 4H), 0.87-0.84 (m, 3H), 0.83 (s, 3H), 0.69-0.58 (m, 4H).

### Synthesis of A-17.4

To a solution of A-17.3 (4.00 g, 10.0 mmol) in THF (30 mL) was added BH₃Me₂S (5 mL, 10 M, 50 mmol) at 25° C. After stirring at 25° C. for 2 hours, aq. NaOH (30 mL, 5.0 M) was added at 0° C., followed by H₂O₂ (15 mL, 30% in water), during which time the reaction temperature was maintained below 15° C. After stirring at 15° C. for another 10 minutes, aq. saturated Na₂S₂O₃ (100 mL) was added in portions at 0° C. The mixture was extracted with EtOAc (3×50 mL). The combined organic phase was washed with aq. saturated Na₂S₂O₃ (100 mL, sat.), dried over Na₂SO₄, filtered, and concentrated. The residue was purified by flash column chromatography (0˜30% of EtOAc in PE) to give A-17.4 (1.10 g, 26%). 1H NMR (400 MHz, CDCl₃) δ_{H} 3.99-3.97 (m, 1H), 3.92-3.83 (m, 1H), 2.22-2.09 (m, 1H), 1.95-1.75 (m, 3H), 1.70-1.47 (m, 12H), 1.40-1.34 (m, 3H), 1.25-1.19 (m, 1H), 1.15-1.04 (m, 2H), 1.15-1.04 (m, 3H), 1.03-0.95 (m, 1H), 0.93-0.85 (m, 4H), 0.83 (s, 3H), 0.73-0.62 (m, 4H).

### Synthesis of A-17.5

To a solution of A-17.4 (500 mg, 1.20 mmol) in DCM (5 mL) was added TEA (242 mg, 2.40 mmol) and N-methyl-imidazole (146 mg, 1.79 mmol) at 20° C. TsCl (295 mg, 1.55 mmol) was added in portions and the reaction temperature was maintained between 20° C.˜30° C. After stirring at 20° C. for 16 hours, to the mixture was added water (10 mL). The organic layer was separated, washed with water (15 mL), dried over Na₂SO₄, filtered, and concentrated to give A-17.5 (600 mg). ¹H NMR (400 MHz, CDCl₃) δ_{H} 7.82-7.73 (m, 2H), 7.39-7.32 (m, 2H), 4.27-4.16 (m, 2H), 3.71-3.69 (m, 2H), 3.25-3.07 (m, 1H), 2.45 (s, 3H), 2.36-2.24 (m, 1H), 1.88-1.71 (m, 3H), 1.70-1.48 (m, 12H), 1.42-1.34 (m, 3H), 1.23-1.18 (m, 3H), 0.91-0.85 (m, 4H), 0.83-0.77 (m, 3H), 0.72-0.57 (m, 4H).

### Synthesis of A-17.6

To a solution of A-17.5 (600 mg, 1.05 mmol) in DMF (10 mL) was added KI (871 mg, 5.25 mol) 25° C. After stirring at 60° C. for 1 hour, to the mixture was added PhSO₂Na (517 mg, 3.15 mmol). After stirring at 60° C. for another 16 hours, the mixture was quenched with aq. 10% NH₄Cl (20 mL) and extracted with EtOAc (2×10 mL). The combined organic phase was washed with aq. 3% LiCl (30 mL), dried over Na₂SO₄, filtered, and concentrated. The residue was purified by flash column (0˜20% of EtOAc in PE) to give A-17.6 (450 mg). A-17.6 (450 mg, 0.832 mmol) was further purified by SFC (Column: DAICEL CHIRALPAK AD (250 mm*30 mm, 10 um); Condition: 0.1% NH₃H₂O ETOH; Begin B: 50%; End B: 50%, FlowRate (mL/min): 70; Injections: 70) to give A-17.6 (230 mg, 51%). ¹H NMR (400 MHz, CDCl₃) δ_{H} 7.99-7.82 (m, 2H), 7.72-7.50 (m, 3H), 3.51-3.27 (m, 2H), 3.06-2.87 (m, 1H), 2.10-2.06 (m, 1H), 1.86-1.74 (m, 2H), 1.70-1.61 (m, 5H), 1.55-1.50 (m, 2H), 1.44-1.31 (m, 5H), 1.27-0.91 (m, 9H), 0.91-0.79 (m, 10H), 0.70-0.60 (m, 1H).

### Synthesis of A-17.7

To a solution of A-17.6 (230 mg, 0.425 mmol) in THF (2 mL) was added n-butyllithium (0.424 mL, 2.5 M in hexane, 1.06 mmol) dropwise under N₂ at -78° C. After stirring at −78° C. for 1 hour, diisopropylamine (57.0 mg, 0.425 mmol) was added at −78° C. under N₂. After stirring at -78° C. for another 1 hour, ((2R)-2-(propan-2-yl) oxirane (73.2 mg, 0.850 mmol) was added at −78° C. under N₂. The mixture was stirred at −78° C. for 2 hour then allowed to warm to 25° C. and stirred at 25° C. for 16 hours. The mixture was quenched with aq. saturated NH₄Cl (10 mL) and extracted with EtOAc (3×5 mL). The combined organic phase was dried over Na₂SO₄, filtered, and concentrated to give A-17.7 (300 mg). ¹H NMR (400 MHz, CDCl₃) δ_{H} 7.99-7.82 (m, 2H), 7.74-7.47 (m, 3H), 3.82-3.71 (m, 1H), 3.36-3.23 (m, 1H), 1.47-1.41 (m, 6H), 1.28-1.22 (m, 15H), 1.12-1.05 (m, 5H), 0.94-0.88 (m, 14H), 0.85-0.81 (m, 6H).

### Synthesis of A-17

A solution of A-17.7 (230 mg, 0.366 mmol) in MeOH (5 mL) was heated at 50° C. Mg powder (350 mg, 14.6 mmol) was added in one portion at 50° C. After the mixture was refluxed at 60° C. for 3 hours, the mixture was quenched with aq. HCl (20 mL, 1 M) and extracted with EtOAc (3×10 mL). The combined organic phase was dried over Na₂SO₄, filtered, and concentrated. The residue was purified by flash column chromatography (0˜20% of EtOAc in PE) to give A-17 (26.2 mg, 15%). ¹H NMR (400 MHz, CDCl₃) δ_{H} 3.32-3.29 (m, 1H), 2.23-2.09 (m, 1H), 2.02-1.92 (m, 1H), 1.89-1.78 (m, 2H), 1.74-1.60 (m, 7H), 1.55-1.48 (m, 4H), 1.43-1.31 (m, 4H), 1.31-1.16 (m, 7H), 1.15-1.00 (m, 4H), 0.95-0.85 (m, 11H), 0.82 (s, 3H), 0.73-0.64 (m, 4H). ¹⁹F NMR (376 MHz, CDCl₃) δ_{F} −64.18 (s). LC-ELSD/MS 30-90AB_2 min_E, purity 99%, MS ESI calcd. for C₂₉H₄₆F₃O₂ [M+H−2H₂O]⁺ 451.4, found 451.4.

### Example 12: Synthesis of (3S,5S,8R,9S,10S,13S,14S,17R)-3-ethyl-10,13-dimethyl-17-((S)-1,1,1-trifluoro-6-hydroxy-6-methylheptan-2-yl)hexadecahydro-1H-cyclopenta[a]phenanthren-3-ol (A-18)

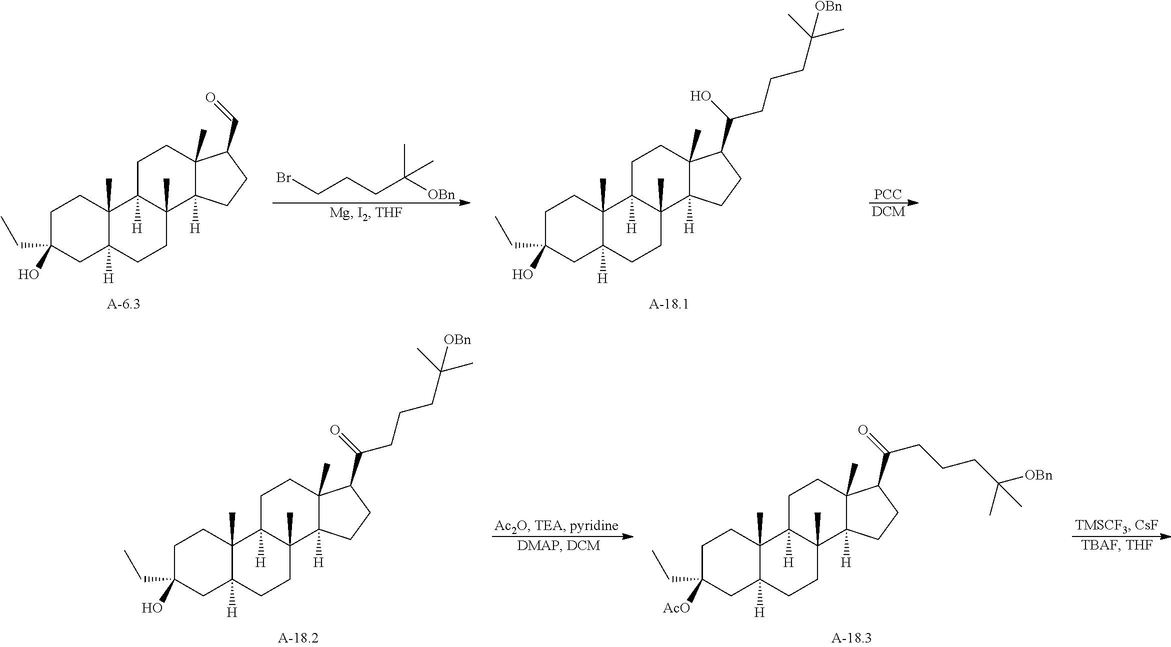

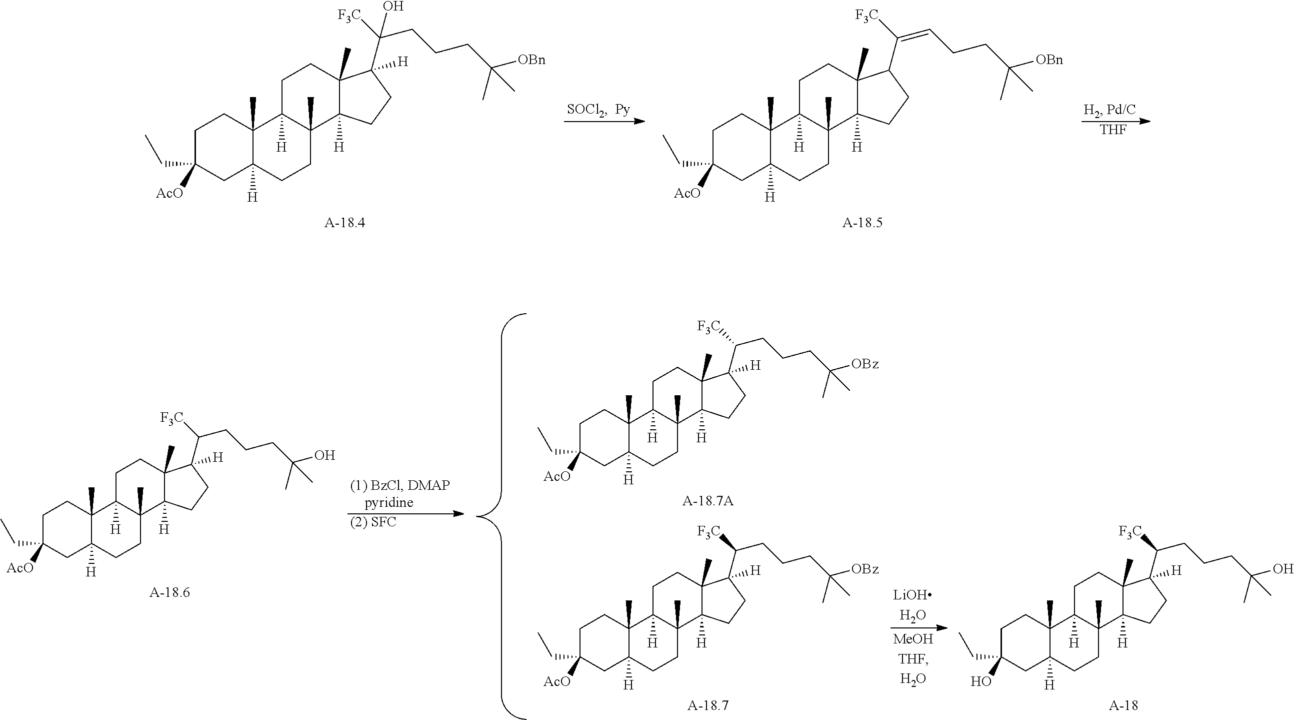

### Synthesis of A-18.1

A solution of (((5-bromo-2-methylpentan-2-yl)oxy)methyl)benzene (20.5 g, 75.6 mmol) in THF (50 mL) was added slowly to a suspension of Mg (3.62 g, 151 mmol) and I₂ (191 mg) in THF (20 mL) under N₂ at 25° C. After stirring for 10 minutes, the mixture was heated at 40° C. for 1 hour. The freshly prepared Grignard reagent was transferred to another round flask under N₂ and to the mixture was added a solution of A-6.3 (6.30 g, 18.9 mmol) in THF (230 mL) at 0° C. The mixture was stirred at 25° C. for 1 hour. The mixture was quenched with sat. aq. NH₄Cl (100 mL) and extracted with EtOAc (3×50 mL). The combined organic phase was washed with brine (2×50 mL), dried over Na₂SO₄, filtered, and concentrated. The residue was purified by flash column chromatography (0˜15% of EtOAc in PE) to give A-18.1 (9.00 g) that was carried on to the next step without further purification.

### Synthesis of A-18.2

To a mixture of A-18.1 (1.20 g, 2.28 mmol) in DCM (15 mL) was added silica gel (1.00 g), and PCC (982 mg, 4.56 mmol). The reaction mixture was stirred at 25° C. for 2 hours. Then, the mixture was filtered, washed with DCM (3×200 mL), and the solvents were evaporated. The residue was purified by flash column chromatography (0˜20% of EtOAc in PE) to give A-18.2 (860 mg, 72%). ¹H NMR (400 MHz, CDCl₃) δ_{H} 7.35-7.29 (m, 4H), 7.25-7.21 (m, 1H), 4.41 (s, 2H), 2.48 (t, J=9.2 Hz, 1H), 2.37 (t, J=7.2 Hz, 2H), 2.17-2.11 (m, 1H), 1.95-1.91 (m, 1H), 1.43-1.27 (m, 10H), 1.26-1.21 (m, 12H), 1.18-0.92 (m, 7H), 0.92-0.85 (m, 5H), 0.82 (s, 3H), 0.76-0.62 (m, 2H), 0.58 (s, 3H).

### Synthesis of A-18.3

To a solution of A-18.2 (1.00 g, 1.91 mmol), DMAP (233 mg, 1.91 mmol), and TEA (2.44 mL, 19.0 mmol) in pyridine (10 mL) and DCM (10 mL) was added drop-wise Ac₂O (1.16 g, 11.4 mmol). The mixture was stirred at 40° C. for 48 hours and then quenched with saturated aqueous NH₄Cl (10 mL). The aqueous phase was extracted with DCM (3×10 mL). The combined organic phase was washed with brine (10 mL), dried over anhydrous Na₂SO₄, filtered, and concentrated. The residue was purified by flash column chromatography (0˜10% of EtOAc in PE) to give A-18.3 (730 mg, 68%). ¹H NMR (400 MHz, CDCl₃) δ_{H} 7.36-7.28 (m, 3H), 7.27-7.21 (m, 2H), 4.41 (s, 2H), 2.51-2.44 (m, 1H), 2.42-2.34 (m, 3H), 2.24-2.08 (m, 2H), 2.00-1.79 (m, 12H), 1.10-0.78 (m, 14H), 1.10-0.78 (m, 12H), 0.71-0.62 (m, 1H), 0.59-0.55 (m, 4H).

### Synthesis of A-18.4

To a solution of A-18.3 (730 mg, 1.29 mmol) in THF (8 mL) was added CsF (391 mg, 2.58 mmol) at 20° C. under N₂. TMSCF₃ (550 mg, 3.87 mmol) was added dropwise at 10° C. The mixture was stirred at 10° C. for 1 hour. To the mixture was added TBAF (10.3 mL, 1 M in THF, 10.3 mmol), H₂O (10 mL), and the mixture was stirred at 20° C. for another 16 hours. The mixture was diluted with EtOAc (50 mL), washed with water (2×50 mL), brine (50 mL), dried over Na₂SO₄, filtered, and concentrated. The residue was purified by flash column chromatography (0˜10% of EtOAc in PE) to give A-18.4 (790 mg). To a suspension of A-18.4 (790 mg, 1.24 mmol) in THF (8 mL) was added TBAF (2.48 mL, 2.48 mmol). The mixture was stirred at 20° C. for 16 hours. The mixture was quenched with H₂O (40 mL) and extracted with EtOAc (2×30 mL). The combined organic phase was washed with water (3×30 mL), dried over Na₂SO₄, filtered, and concentrated. purification by flash column chromatography (0˜15% of EtOAc in PE) provided A-18.4 (640 mg). ¹H NMR (400 MHz, CDCl₃) δ_{H} 7.36-7.28 (m, 4H), 7.25-7.19 (m, 1H), 4.40 (s, 2H), 2.01-1.76 (m, 18H), 1.56-1.36 (m, 10H), 1.20-0.91 (m, 8H), 0.89-0.78 (m, 13H), 0.60-0.43 (m, 1H). ¹⁹F NMR (376 MHz, CDCl₃) δ_{F} −75.57 (s).

### Synthesis of A-18.5

To a solution of A-18.4 (100 mg, 0.16 mmol) in pyridine (2 mL) was added SOCI2 (0.11 mL, 1.57 mmol) and the reaction mixture was stirred at 80° C. under N₂ for 25 minutes. The mixture was cooled to 25° C. and aq. saturated NaHCO₃ (20 mL) was added. The mixture was extracted with EtOAc (2×20 mL). The combined organic layer was washed with brine (20 mL), dried over Na₂SO₄, filtered, and concentrated. The residue was purified by flash column chromatography (0˜3% EtOAc in PE) to give A-18.5 (95.0 mg), which was carried directly into the next step without further purification.

### Synthesis of A-18.6

To a mixture of A-18.5 (95.0 mg, 0.15 mmol) in THF (2 mL) was added Pd/C (300 mg, 10%, dry). The suspension was degassed under vacuum and purged with H₂ four times. The mixture was stirred under H₂ (50 psi) at 50° C. for 16 hours. The mixture was filtered and the solid was washed with THF (3×10 mL). The filtrate was concentrated to give A-18.6 (90.0 mg), which was carried directly into the next step without further purification.

### Synthesis of A-18.7 & A-18.7a

To a solution of A-18.6 (360 mg, 0.68 mmol) and DMAP (83.1 mg, 0.68 mmol) in pyridine (3 mL) was added benzoyl chloride (191 mg, 1.36 mmol) dropwise at 0° C. The mixture was stirred at 25° C. for 72 hours and then poured into ice-water (10 mL). The aqueous phase was extracted with EtOAc (3×10 mL). The combined organic phase was washed with brine (10 mL), dried over anhydrous Na₂SO₄, filtered, and concentrated. The residue was purified by flash column chromatography (0˜5% of EtOAc in PE) and further purified by SFC (Column: DAICEL CHIRALCEL OD-H (250 mm*30 mm, 5 um); Condition: 0.1% NH₃H₂O ETOH; Begin B: 15%; End B: 15%; FlowRate (ml/min): 60; Injections: 90) to afford A-18.7 (30 mg, 8%) and A-18.7A (140 mg, 40%).

A-18.7: ¹H NMR (400 MHz, CDCl₃) δ_{H} 8.00-7.92 (m, 2H), 7.57-7.48 (m, 1H), 7.45-7.37 (m, 2H), 3.74 (t, J=6.4 Hz, 1H), 2.17-1.63 (m, 20H), 1.53-0.92 (m, 18H), 0.87-0.80 (m, 7H), 0.63 (s, 3H), 0.58-0.47 (m, 1H). ¹⁹F NMR (376 MHz, CDCl₃) δ_{F} −64.52 (s).

A-18.7a: ¹H NMR (400 MHz, CDCl₃) δ_{H} 7.97 (d, J=7.2 Hz, 2H), 7.60-7.47 (m, 1H), 7.46-7.34 (m, 2H), 2.02-1.93 (m, 6H), 1.91-1.58 (m, 16H), 1.56-0.88 (m, 18H), 0.85 (s, 3H), 0.81 (t, J=7.6 Hz, 3H), 0.69-0.62 (m, 1H), 0.60 (s, 3H). ¹⁹F NMR (376 MHz, CDCl₃) δ_{F} −67.71 (s).

### Synthesis of A-18

To a solution of A-18.7 (30 mg, 0.047 mmol) in THF (0.5 mL) was added MeOH (1 mL), H₂O (0.5 mL), and LiOH· H₂O (200 mg). The mixture was stirred at 25° C. for 72 hours. The mixture was diluted with water (10 mL) and extracted with EtOAc (2×10 mL). The combined organic layer was dried over Na₂SO₄, filtered, and concentrated. The residue was purified by flash column chromatography (0˜40% of EtOAc in PE) to give A-18 (11.0 mg, 37%). ¹H NMR (400 MHz, CDCl₃) δ_{H} 2.18-2.05 (m, 1H), 1.90-1.55 (m, 11H), 1.54-1.28 (m, 11H), 1.27-1.16 (m, 11H), 1.13-0.93 (m, 4H), 0.88 (t, J=7.2 Hz, 4H), 0.83 (s, 3H), 0.72-0.60 (m, 4H). ¹⁹F NMR (376 MHz, CDCl₃) δ_{F} −64.49 (s). LC-ELSD/MS 30-90AB_2 min_E, purity 99%, MS ESI calcd. for C₂₉H₄₉F₃O₂ [M−2H₂O+H]⁺ 451.4, found 451.4.

### Example 13: (3S,5S,8R,9S,10S,13S,14S,17R)-3-ethyl-10,13-dimethyl-17-((2R,5R)-1,1,1-trifluoro-5-hydroxy-6-methylheptan-2-yl)hexadecahydro-1H-cyclopenta[a]phenanthren-3-ol (A-19)

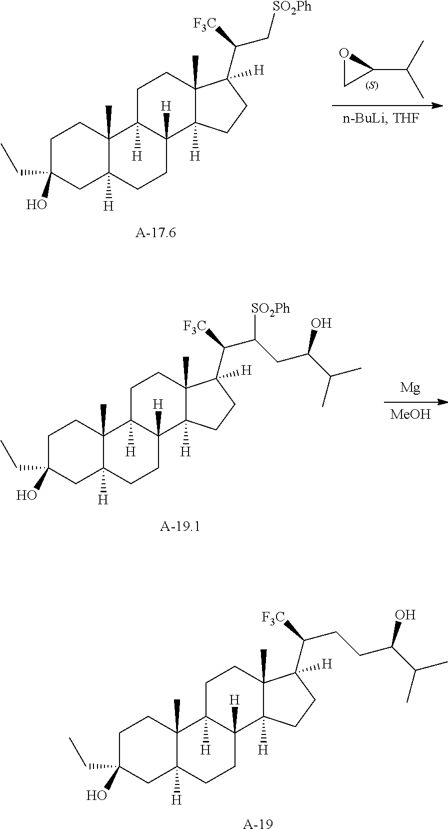

### Synthesis of A-19.1

To a solution of A-17.6 (220 mg, 0.406 mmol) in THF (2 mL) was added n-butyllithium (0.404 mL, 2.5 M in hexane, 1.01 mmol) dropwise under N₂ at −78° C. After stirring at -78° C. for 1 h, ((2S)-2-(propan-2-yl) oxirane (69.9 mg, 0.812 mmol) was added at 78° C. under N₂. After stirring at −78° C. for 2 h, the mixture was allowed to warm to 25° C. and stirred at 25° C. for 16 h. The mixture was quenched with aq. saturated NH₄Cl (10 mL) and extracted with EtOAc (3×5 mL). The combined organic phase was dried over Na₂SO₄, filtered, and concentrated to give A-19.1 (230 mg, crude) as a yellow solid. ¹H NMR (400 MHz, CDCl₃) δ_{H} 7.91 (d, J=7.2 Hz, 2H), 7.72-7.48 (m, 3H), 3.93-3.80 (m 1H), 3.52-3.25 (m, 2H), 1.97-1.86 (m, 2H), 1.45-1.19 (m, 16H), 1.16-0.97 (m, 7H), 0.91-0.87 (m, 8H), 0.86-0.75 (m, 8H), 0.73-0.62 (m, 4H).

### Synthesis of A-19

A solution of A-19.1 (230 mg, 0.366 mmol) in MeOH (5 mL) was heated at 50° C. Mg powder (350 mg, 14.6 mmol) was added in one portion at 50° C. After the mixture was refluxed at 60° C. for 3 h, the mixture was quenched with HCl (20 mL, 1 M) and extracted with EtOAc (3×10 mL). The combined organic phase was dried over Na₂SO₄, filtered, and concentrated. The residue was purified by flash column chromatography (0˜20% of EtOAc in PE) to give A-19 (65.8 mg, 36.9%) as a white solid. ¹H NMR (400 MHz, CDCl₃) δ_{H} 3.27-3.25 (m, 1H), 2.20-2.07 (m, 1H), 2.01-1.88 (m, 2H), 1.86-1.76 (m, 1H), 1.71-1.59 (m, 7H), 1.56-1.49 (m, 5H), 1.43-1.29 (m, 5H), 1.28-1.16 (m, 6H), 1.14-0.99 (m, 4H), 0.93-0.89 (m, 7H), 0.89-0.85 (m, 3H), 0.82 (s, 3H), 0.71-0.64 (m, 4H). ¹⁹F NMR (376.5 MHz, CDCl₃) &F-64.486. LC-ELSD/MS purity 100%, MS ESI calcd. for C₂₉H₄₆F₃O₂ [M+H-2H₂O]⁺ 451.4, found 451.4.

### Example 14: Synthesis of (3S,5S,8R,9S,10S,13R,14S,17R)-3-ethyl-17-((S)-7-hydroxy-7-methyloctan-3-yl)-10,13-dimethylhexadecahydro-1H-cyclopenta[a]phenanthren-3-ol (A-20)

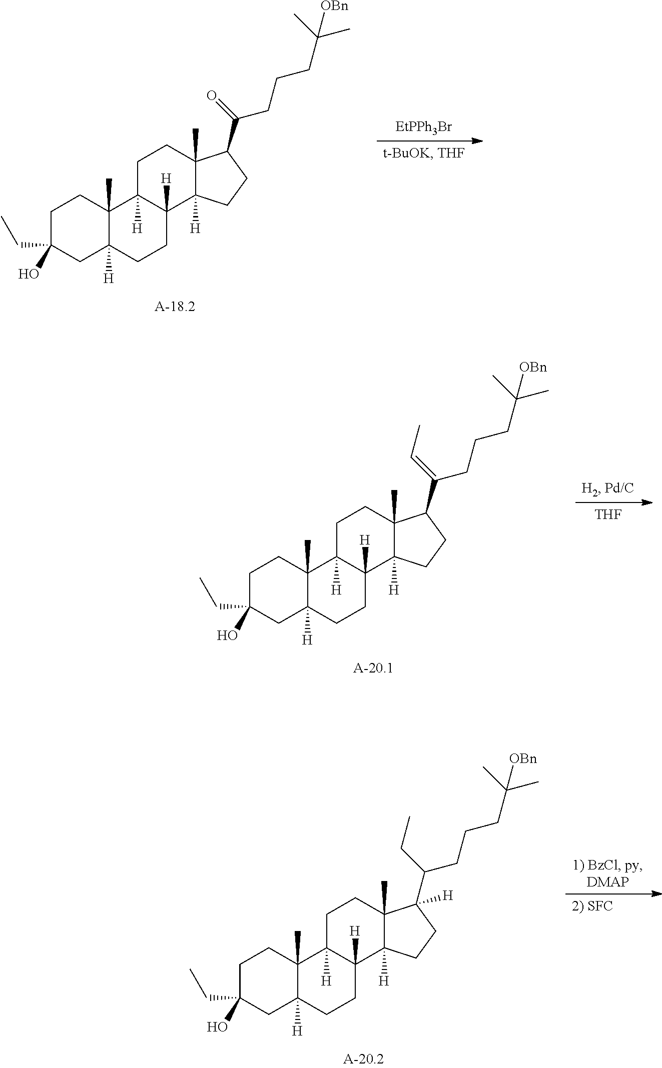

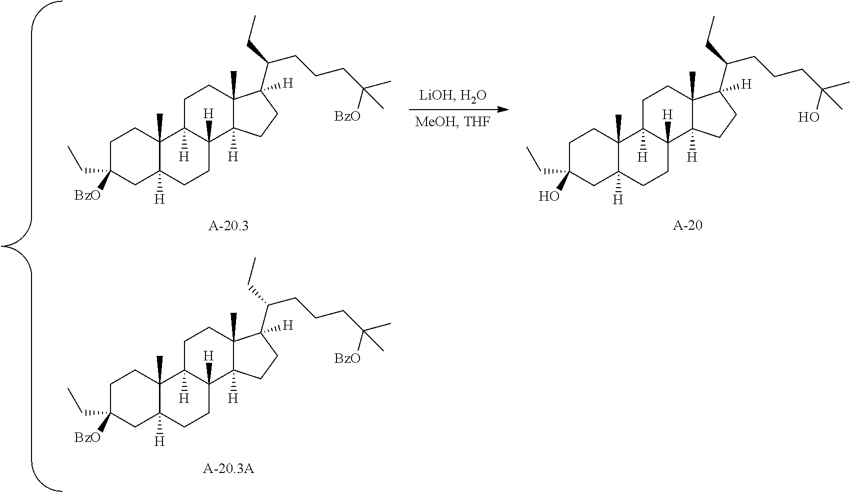

### Synthesis of A-20.1

To a mixture of EtPPh₃Br (1.82 g, 4.92 mmol) in THF (8 mL) was added t-BuOK (552 mg, 4.92 mmol) at 25° C. under N₂. The resulting mixture was stirred at 40° C. for 30 minutes. A-18.2 (0.86 g, 1.64 mmol) in THF (2 mL) was added in portions below 40° C. The reaction mixture was stirred at 40° C. for 3 hours. The reaction mixture was quenched with aqueous 10% NH₄Cl (20 mL), extracted with EtOAc (2×30 mL), washed with brine (20 mL), dried over Na₂SO₄, filtered, and concentrated. The residue was purified by flash column chromatography on silica gel (0˜20% of EtOAc in PE) to give A-20.1 (40.0 mg, 5%).

### Synthesis of A-20.2

To a mixture of A-20.1 (140 mg, 0.26 mmol) in THF (5 mL)) was added Pd/C (300 mg, 10%, dry). The suspension was degassed under vacuum and purged with H₂ three times. The mixture was stirred under H₂ (50 psi) at 50° C. for 60 hours. The mixture was filtered, the solid washed with THF (3×20 mL), and the filtrate was concentrated. The residue was purified by flash column chromatography (0˜20% EtOAc in PE) to give A-20.2 (80.0 mg, 69%), which was carried directly into the next step without further purification.

### Synthesis of A-20.3 & A-20.3a

To a solution of A-20.2 (80.0 mg, 0.18 mmol) and DMAP (21.8 mg, 0.18 mmol) in pyridine (1 mL) was added dropwise benzoyl chloride (125 mg, 0.90 mmol) at 0° C. The mixture was stirred at 20° C. for 72 hours and then poured into ice-water (10 mL). The aqueous phase was extracted with EtOAc (3×10 mL). The combined organic phase was washed with brine (10 mL), dried over anhydrous Na₂SO₄, filtered, and concentrated. The residue was purified by flash column chromatography (0˜2% of EtOAc in PE) and the diastereomers were separated by SFC (Column: DAICEL CHIRALCEL OD-H (250 mm*30 mm, 5 um); Condition: 0.1% NH₃H₂O EtOH; Begin B: 35%; End B: 35%; FlowRate (mL/min): 60; Injections: 80) to afford A-20.3 (22.0 mg, 21%) and A-20.3a (30.0 mg, 28%).

A-20.3: ¹H NMR (400 MHz, CDCl₃) δ_{H} 8.00-7.94 (m, 4H), 7.56-7.48 (m, 2H), 7.45-7.38 (m, 4H), 2.17-2.08 (m, 4H), 1.98-1.66 (m, 10H), 1.50-1.11 (m, 18H), 1.10-0.96 (m, 6H), 0.94-0.85 (m, 7H), 0.78 (t, J=7.2 Hz, 3H), 0.70-0.56 (m, 4H).

A-20.3a: ¹H NMR (400 MHz, CDCl₃) δ_{H} 8.00-7.94 (m, 4H), 7.56-7.48 (m, 2H), 7.45-7.37 (m, 4H), 2.18-2.07 (m, 4H), 1.97-1.67 (m, 10H), 1.44-1.15 (m, 18H), 1.13-0.96 (m, 6H), 0.92-0.85 (m, 7H), 0.78 (t, J=7.2 Hz, 3H), 0.70-0.61 (m, 4H).

### Synthesis of A-20

To a solution of A-20.3 (22.0 mg, 0.030 mmol) in THF (1 mL) was added MeOH (0.5 mL), H₂O (0.5 mL), and LiOH·H₂O (70.0 mg). The mixture was stirred at 60° C. for 32 hours. The mixture was then diluted with water (10 mL) and extracted with EtOAc (2×10 mL). The combined organic layer was dried over Na₂SO₄, filtered, and concentrated. The residue was purified by flash column chromatography (0˜20% of EtOAc in PE) to give A-20 (8.6 mg, 39%). ¹H NMR (400 MHz, CDCl₃) δ_{H} 1.95-1.85 (m, 1H), 1.83-1.57 (m, 5H), 1.53-1.25 (m, 17H), 1.24-1.15 (m, 12H), 1.12-0.92 (m, 6H), 0.88 (t, J=7.2 Hz, 3H), 0.82 (s, 3H), 0.78 (t, J=7.2 Hz, 3H), 0.69-0.59 (m, 4H). LC-ELSD/MS 30-90AB_2 min_E, purity>99%, MS ESI calcd. for C₃₀H₅₄O₂ [M−2H₂O+H]⁺ 411.4, found 411.4.

### Example 15: Synthesis of (3S,5S,8R,9S,10S,13S,14S,17S)-3-ethyl-10,13-dimethyl-17-((1R,4R)-5,5,5-trifluoro-4-hydroxy-1-methoxy-4-methylpentyl) hexadecahydro-1H-cyclopenta[a]phenanthren-3-ol (A-23); (3S,5S,8R,9S,10S,13S,14S,17S)-3-ethyl-10,13-dimethyl-17-((1R,4S)-5,5,5-trifluoro-4-hydroxy-1-methoxy-4-methylpentyl) hexadecahydro-1H-cyclopenta[a]phenanthren-3-ol (A-24); (3S,5R,8R,9S,10S,13S,14S,17S)-3-ethyl-10,13-dimethyl-17-((1R,4R)-5,5,5-trifluoro-4-hydroxy-1-methoxy-4-methylpentyl) hexadecahydro-1H-cyclopenta[a]phenanthren-3-ol (A-25); and (3S,5R,8R,9S,10S,13S,14S,17S)-3-ethyl-10,13-dimethyl-17-((1R,4S)-5,5,5-trifluoro-4-hydroxy-1-methoxy-4-methylpentyl) hexadecahydro-1H-cyclopenta[a]phenanthren-3-ol (A-26)

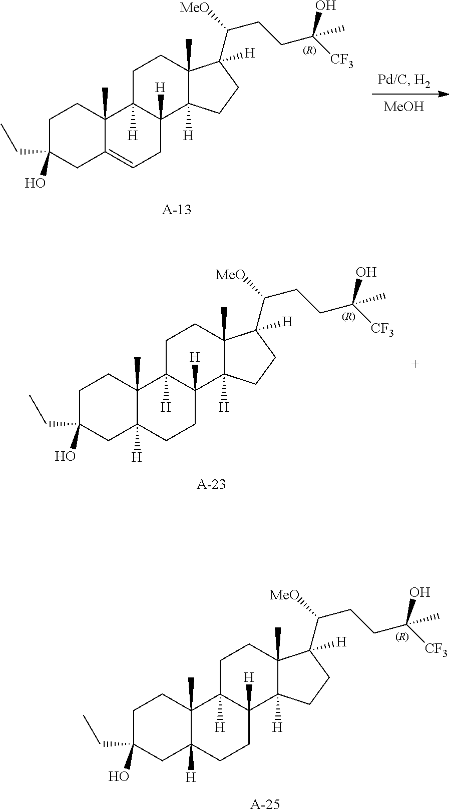

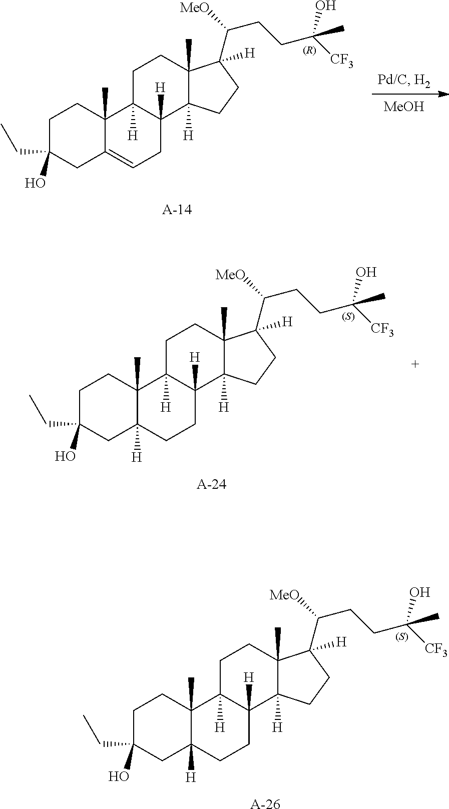

### Synthesis of A-23 and A-25

To a solution of A-13 (100 mg, 0.205 mmol) in THF (10 mL) was added Pd/C (50 mg, 10%, dry) under N₂. The suspension was degassed under vacuum and purged with H₂ three times. The mixture was stirred under H₂ (50 psi) at 50° C. for 24 h. The reaction mixture was filtered through a pad of Celite and washed with THF (2×50 mL), concentrated, and purified by silica gel chromatography (0˜40% of EtOAc in PE) to give A-23 (8.8 mg, 9%) and A-25 (4.9 mg, 5%).

A-23: ¹H NMR (400 MHz, CDCl₃) δ_{H} 3.32 (s, 3H), 3.28-3.22 (m, 1H), 2.00-1.61 (m, 9H), 1.50-1.27 (m, 12H), 1.26-0.91 (m, 12H), 0.88 (t, J=7.6 Hz, 3H), 0.83 (s, 3H), 0.68 (s, 3H), 0.67-0.60 (m, 1H). ¹⁹F NMR (376 MHz, CDCl₃) δ_{F} −81.25 (s). LC-ELSD/MS 30-90AB_2 min_E, purity>99%, MS ESI calcd. for C₂₇H₄₂F₃O [M−MeOH−H₂O+H]⁺ 439.3, found 439.3.

A-25: ¹H NMR (400 MHz, CDCl₃) δ_{H} 3.32 (s, 3H), 3.29-3.21 (m, 1H), 2.01-1.72 (m, 6H), 1.50-1.35 (m, 9H), 1.34-1.24 (m, 10H), 1.23-0.98 (m, 9H), 0.97 (s, 3H), 0.91 (t, J=7.6 Hz, 3H), 0.68 (s, 3H). ¹⁹F NMR (376 MHz, CDCl₃) δ_{F} −81.05 (s). LC-ELSD/MS 30-90AB_2 min_E, purity>99%, MS ESI calcd. for C₂₇H₄₂F₃O [M−MeOH−H₂O+H]⁺ 439.3, found 439.3.

### Synthesis of A-24 and A-26

To a mixture of A-14 (500 mg, 1.13 mmol) in MeOH (10 mL) was added Pd/C (500 mg, 10%, dry). The suspension was degassed under vacuum and purged with H₂ three times. The mixture was stirred under H₂ (50 psi) at 50° C. for 72 h. The mixture was filtered, washed with THF (3×50 mL), and concentrated. The residue was purified by silica gel chromatography (0-40% of EtOAc in PE) to give A-24 (123.2 mg, 30%) and A-26 (123.9 mg, 34%).

A-24: ¹H NMR (400 MHz, CDCl₃) δ_{H} 3.32 (s, 3H), 3.30-3.24 (m, 1H), 3.22-3.12 (m, 1H), 2.00-1.80 (m, 3H), 1.72-1.58 (m, 8H), 1.50-1.33 (m, 7H), 1.32 (s, 3H), 1.31-0.90 (m, 11H), 0.88 (t, J=7.6 Hz, 3H), 0.83 (s, 3H), 0.68 (s, 3H), 0.66-0.62 (m, 1H) ¹⁹F NMR (376 MHz, CDCl₃) δ_{F} −83.19 (s). LC-ELSD/MS 30-90AB_2 min_E, purity>99%, MS ESI calcd. for C₂₇H₄₂F₃O [M−MeOH−H₂O+H]⁺ 439.3, found 439.3.

A-26: ¹H NMR (400 MHz, CDCl₃) δ_{H} 3.44-3.35 (m, 1H), 3.32 (s, 3H), 3.31-3.24 (m, 1H), 2.01-1.83 (m, 4H), 1.81-1.62 (m, 5H), 1.50-1.35 (m, 8H), 1.35-1.29 (m, 6H), 1.28-1.02 (m, 10H), 0.97 (s, 3H), 0.92 (t, J=7.6 Hz, 3H), 0.68 (s, 3H) ¹⁹F NMR (376 MHz, CDCl₃) δ_{F} −83.26 (s). LC-ELSD/MS 30-90AB_2 min_E, purity>99%, MS ESI calcd. for C₂₇H₄₂F₃O [M−MeOH−H₂O+H]⁺ 439.3, found 439.3.

### Example 16: Synthesis of (2S,4aS,4bS,6aR,7S,7aS,8aR,8bS,8cR,10aS)-2-ethyl-7-((R)-6-hydroxy-6-methylheptan-2-yl)-4a,6a-dimethyloctadecahydrocyclopropa[4,5]cyclopenta[1,2-a]phenanthren-2-ol (B-1)

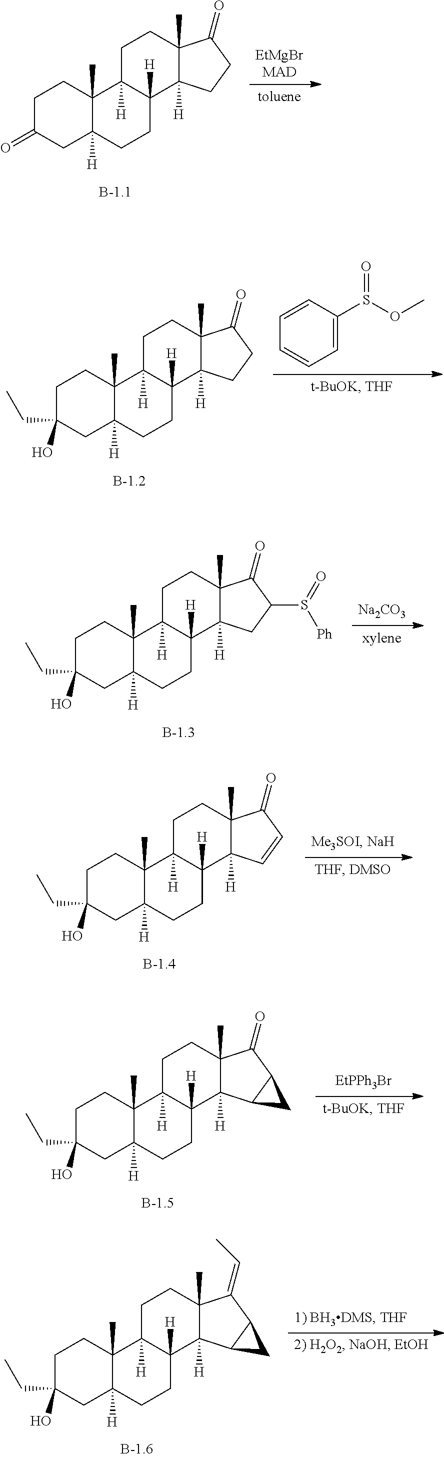

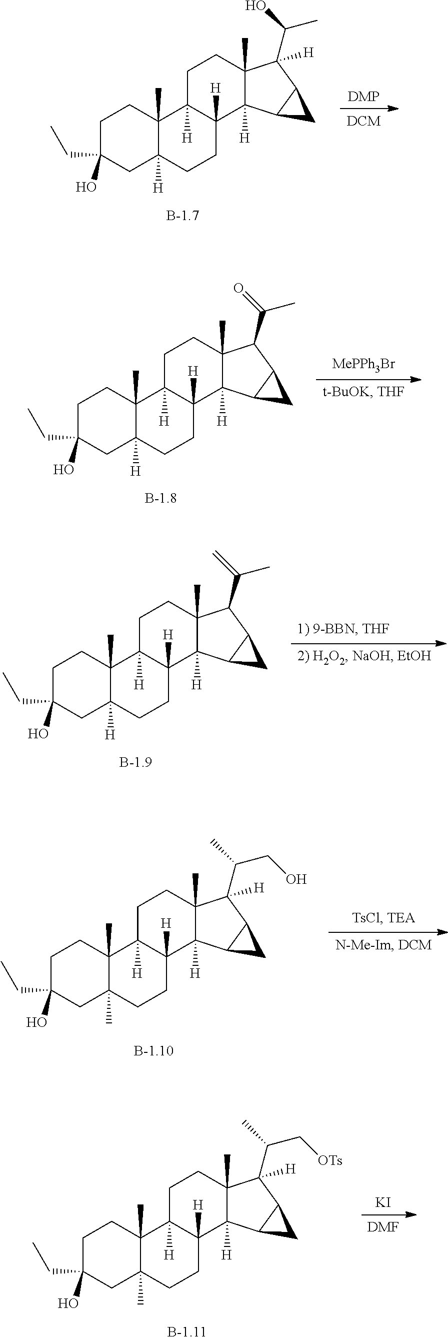

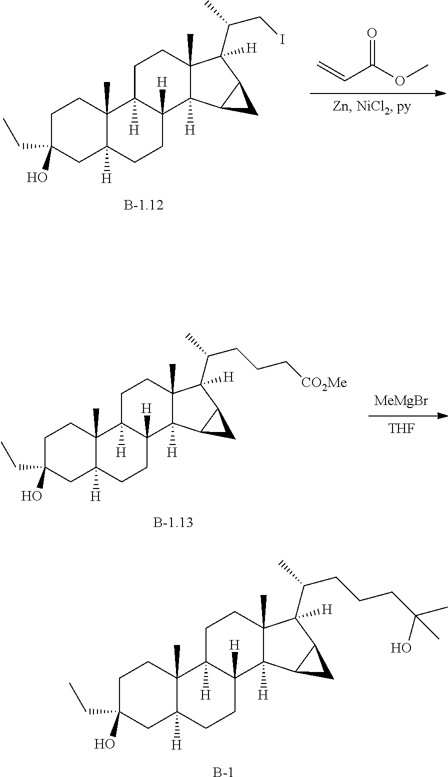

### Synthesis of B-1.2

To a solution of BHT (68.7 g, 312 mmol) in toluene (100 mL) under N₂ at 0° C. was added trimethylaluminum (2 M in toluene, 78 mL, 156 mmol) dropwise. After stirring at 25° C. for 1 hour, to the above solution was added a solution of B-1.1 (15.0 g, 52.0 mmol, reported in [Chemical Science, 2019, vol. 10, #32, p. 7542-7548]) in DCM (50 mL) dropwise at -70° C. After stirring at -70° C. for 1 hour under N₂, EtMgBr (52 mL, 156 mmol, 3M in ethyl ether) was added dropwise at −70° C. The resulting solution was stirred at −70° C. for another 2 hours, poured into saturated citric acid (500 mL) at below 10° C. The aqueous phase was extracted with DCM (3×100 mL). The organic phase was washed with brine (2×50 mL), dried over anhydrous Na₂SO₄, filtered, and concentrated. The residue was triturated with MeOH (1.5 L) at 25° C. to give B-1.2 (14.0 g). ¹H NMR (400 MHz, CDCl₃) δ_{H} 2.50-2.38 (m, 1H), 2.12-2.00 (m, 1H), 1.97-1.88 (m, 1H), 1.83-1.74 (m, 2H), 1.70-1.48 (m, 9H), 1.41-1.33 (m, 3H), 1.29-1.23 (m, 3H), 1.21-1.07 (m, 2H), 1.05-0.94 (m, 2H), 0.90-0.81 (m, 9H), 0.78-0.68 (m, 1H).

### Synthesis of B-1.3

To a solution of t-BuOK (31.6 g, 282 mmol) in THF (800 mL) was added B-1.2 (45.0 g, 141 mmol) at 15° C. under N₂. The mixture was stirred for 10 mins and then methyl benzenesulfinate (35.6 mL, 282 mmol) was added. After the mixture stirring at 30° C. for 0.5 hour, the mixture was quenched with H₂O (500 mL) and extracted with EtOAc (2×300 mL). The organic layer was separated, dried over Na₂SO₄, filtered, and concentrated in vacuum to give B-1.3 (75.0 g), which was used directly in the next step.

### Synthesis of B-1.4

To a mixture of B-1.3 (71.0 g, 160 mmol) in xylene (1000 mL) was added Na₂CO₃ (259 g, 2400 mmol) in portions. After the reaction mixture as stirred at 125° C. for 12 hours under N₂, the mixture was filtered and concentrated. The residue was purified by silica gel chromatography (0-30% of EtOAc in PE/DCM (1/1)) to give B-1.4 (31.0 g). ¹H NMR (400 MHz, CDCl₃) δ_{H} 7.51 (d, J=6.0 Hz, 1H), 6.07-5.98 (m, 1H), 2.26 (d, J=11.6 Hz, 1H), 2.01-1.59 (m, 6H), 1.58-1.08 (m, 12H), 1.05 (s, 3H), 1.03-0.75 (m, 8H).

### Synthesis of B-1.5

To a mixture of trimethylsulfoxonium iodide (32.1 g, 146 mmol) in DMSO (300 mL) and THF (150 mL) was added NaH (5.83 g, 146 mmol, 60% in oil) and the mixture was stirred at 0° C. for 1 hour under N₂. To the mixture was added a solution of B-1.4 (31.0 g, 97.9 mmol) in DMSO (200 mL) at 0° C. and stirring was continued at 20° C. for 16 hours. The reaction was treated with water (300 mL) and extracted with EtOAc (2×300 mL). The combined organic phase was washed with water (2×300 mL), brine (500 mL), dried over anhydrous Na₂SO₄, filtered, and concentrated. The residue was purified by column chromatography (0-20% of EtOAc in PE) to give B-1.5 (21.0 g, 65%). ¹H NMR (400 MHz, CDCl₃) δ_{H} 1.99-1.89 (m, 3H), 1.75-1.57 (m, 10H), 1.43-1.29 (m, 6H), 1.18-1.00 (m, 4H), 0.95 (s, 3H), 0.91-0.85 (m, 6H), 0.83-0.70 (m, 2H).

### Synthesis of B-1.6

To a solution of PPh₃EtBr (70.5 g, 190 mmol) in THF (250 mL) was added t-BuOK (21.3 g, 190 mmol) and the reaction mixture was stirred for 0.5 hour at 45° C. A solution of B-1.5 (21.0 g, 63.5 mmol) in THF (200 mL) was added into the reaction at 45° C. The reaction mixture was stirred for 12 hours at 45° C., then poured into saturated NH₄Cl (500 mL) and extracted with EtOAc (2×200 mL). The combined organic phase was washed with saturated brine (500 mL), dried over anhydrous Na₂SO₄, filtered, and concentrated. The mixture was triturated with (MeOH/H₂O, 1/1, 1 L) to give B-1.6 (22.0 g). ¹HNMR (400 MHz, CDCl₃) δ_{H} 5.35 (q, J=7.2 Hz, 1H), 2.15-2.07 (m, 1H), 1.95-1.87 (m, 1H), 1.79-1.73 (m, 1H), 1.68 (d, J=7.2 Hz, 3H), 1.62-1.53 (m, 10H), 1.38-1.11 (m, 9H), 0.98 (s, 3H), 0.91-0.85 (m, 7H), 0.76-0.70 (m, 1H), 0.48-0.38 (m, 1H).

### Synthesis of B-1.7

To a solution of B-1.6 (21.0 g, 61.3 mmol) in THF (200 mL) was added BH₃·Me₂S (12.2 ml, 10M, 122 mmol) under N₂, and the mixture was stirred at 20° C. for 12 hours. The mixture was cooled to 0° C. and EtOH (43.3 mL, 735 mmol) and NaOH (147 mL, 5M, 735 mmol) were added. H₂O₂ (73.5 mL, 10M, 735 mmol) was added dropwise at 15° C. The mixture was stirred at 20° C. for 1 hour and then poured into Na₂S₂O₃ (500 mL, sat.) and stirred for 30 mins. The aqueous layer was extracted with EtOAc (2×300 mL). The combined organic layer was washed with saturated brine (500 mL), dried over anhydrous Na₂SO₄, filtered, and concentrated to give B-1.7 (16.0 g). ¹H NMR (400 MHz, CDCl₃) δ_{H} 3.85-3.77 (m, 1H), 1.96-1.86 (m, 2H), 1.57-1.55 (m, 7H), 1.39-1.28 (m, 9H), 1.25-1.23 (m, 3H), 1.11-0.98 (m, 5H), 0.89-0.83 (m, 9H), 0.73 (s, 3H), 0.24-0.16 (m, 1H).

### Synthesis of B-1.8

To a solution of B-1.7 (16.0 g, 44.3 mmol) in DCM (300 mL) was added DMP (37.5 g, 88.6 mmol). The mixture was stirred at 20° C. for 30 minutes and then quenched with saturated aqueous NaHCO₃ (300 mL). The organic phase was separated and washed with saturated aqueous NaHCO₃/Na₂S₂O₃ (1:1, 2×300 mL), brine (500 mL), dried over anhydrous Na₂SO₄, filtered, and concentrated. The residue was purified by flash column chromatography (0˜20% of EtOAc in PE) to provide B-1.8 (6.40 g, 70%). ¹H NMR (400 MHz, CDCl₃) δ_{H} 2.72 (d, J=4.0 Hz, 1H), 2.20 (s, 3H), 2.16-1.58 (m, 10H), 1.41-1.07 (m, 12H), 0.91-0.83 (m, 7H), 0.78-0.67 (m, 4H), 0.44-0.35 (m, 1H).

### Synthesis of B-1.9

To a solution of PPh₃MeBr (10.1 g, 28.4 mmol) in THF (100 mL) was added t-BuOK (3.18 g, 28.4 mmol) and the reaction mixture was stirred for 0.5 hour at 50° C. A solution of B-1.8 (3.40 g, 9.48 mmol) in THF (50 mL) was added at 50° C. and the mixture was stirred for 3 hours at 50° C. The mixture was poured into saturated NH₄Cl (300 mL) and extracted with EtOAc (2×100 mL). The combined organic phase was washed with brine (300 mL), dried over anhydrous Na₂SO₄, filtered, and concentrated. The mixture was triturated from (MeOH/H₂O, 1/1, 150 mL) to give B-1.9 (3.40 g). ¹H NMR (400 MHz, CDCl₃) δ_{H} 5.14-5.06 (m, 1H), 4.88-4.80 (m, 1H), 2.45-2.35 (m, 1H), 1.97-1.87 (m, 1H), 1.78-1.72 (m, 4H), 1.67-1.59 (m, 3H), 1.55-1.24 (m, 10H), 1.23-0.93 (m, 7H), 0.89 (t, J=7.6 Hz, 3H), 0.83 (s, 3H), 0.76-0.60 (m, 5H), 0.28-0.19 (m, 1H).

### Synthesis of B-1.10

To a solution of B-1.9 (200 mg, 0.561 mmol) in THF (5 mL) was added 9-BBN (5.60 ml, 0.5M in THF, 2.80 mmol) under N₂ and the reaction mixture was stirred at 25° C. for 12 hours. EtOH (0.989 mL, 16.8 mmol) and NaOH (3.36 mL, 5M, 16.8 mmol) were added to the mixture. H₂O₂ (1.68 mL, 10M, 16.8 mmol) was added dropwise at 15° C. and the mixture was stirred at 70° C. for 1 hour. The mixture was cooled and poured into Na₂S₂O₃ (50 mL, sat.) and stirred for 30 mins. The aqueous layer was extracted with EtOAc (2×50 mL). The combined organic layers were washed with brine (100 mL), dried over anhydrous Na₂SO₄, filtered, and concentrated. The residue was purified by flash column chromatography (15-25% EtOAc in PE) twice to give B-1.10 (70.0 mg, 33%). ¹H NMR (400 MHz, CDCl₃) δ_{H} 3.91-3.78 (m, 1H), 3.66-3.54 (m, 1H), 1.97-1.87 (m, 1H), 1.79-1.68 (m, 2H), 1.64-1.56 (m, 6H), 1.54-1.26 (m, 9H), 1.21-1.06 (m, 6H), 1.04-0.94 (m, 4H), 0.91-0.83 (m, 6H), 0.79-0.60 (m, 5H), 0.17-0.07 (m, 1H), LC-ELSD/MS purity >99%, MS ESI calcd. for C25H₄₂₀₂ [M−H₂O+H]⁺ 357.3, found 357.3.

### Synthesis of B-1.11

To a solution of B-1.10 (3.50 g, 9.34 mmol) in DCM (35 mL) was added N-methylimidazole (1.52 g, 18.6 mmol), TEA (3.61 mL, 28.0 mmol), and TsCl (2.66 g, 14.0 mmol). The mixture was stirred at 25° C. for 1 hour and then poured into NaHCO₃ (40 mL, saturated). The aqueous phase was extracted with DCM (2×40 mL). The combined organic phase was washed with brine (40 mL), dried over anhydrous Na₂SO₄, filtered, and concentrated. Purification by SFC (Column: DAICEL CHIRALPAK AD (250 mm*50 mm, 10 um); Condition: 0.1% NH₃H₂O ETOH; Begin B: 50%; End B: 50%; Flow Rate (ml/min): 200) provided B-1.11 (3.20 g, 82%). ¹H NMR (400 MHz, CDCl₃) δ_{H} 7.80 (d, J=8.4 Hz, 2H), 7.34 (d, J=8.0 Hz, 2H), 4.22-4.04 (m, 2H), 2.44 (s, 3H), 1.93-1.76 (m, 2H), 1.61-1.51 (m, 7H), 1.43-0.96 (m, 16H), 0.96-0.92 (m, 3H), 0.90-0.85 (m, 3H), 0.82 (s, 3H), 0.67 (s, 3H), 0.56-0.48 (m, 1H), 0.08-0.02 (m, 1H).

### Synthesis of B-1.12

To a solution of B-1.11 (3.20 g, 6.05 mmol) in DMF (20 mL) was added KI (4.81 g, 29.0 mmol) at 25° C. and the mixture was stirred at 60° C. for 5 hours. The mixture was poured into water (20 mL), stirred for 20 min, and the aqueous phase was extracted with EtOAc (3×20 mL). The combined organic phase was washed with brine (2×20 mL), dried over anhydrous Na₂SO₄, filtered, and concentrated to give B-1.12 (2.10 g, 72%). ¹H NMR (400 MHz, CDCl₃) δ_{H} 3.55-3.49 (m, 1H), 3.48-3.42 (m, 1H), 1.98-1.85 (m, 1H), 1.69-1.51 (m, 11H), 1.38-1.11 (m, 12H), 1.00-0.97 (m, 3H), 0.91-0.86 (m, 3H), 0.84 (s, 3H), 0.76 (s, 3H), 0.74-0.57 (m, 2H), 0.17-0.09 (m, 1H).

### Synthesis of B-1.13

To a mixture of Zn powder (377 mg, 5.77 mmol) in dry pyridine (8 mL) was added slowly methyl acrylate (496 mg, 5.77 mmol) and NiCl₂ (159 mg, 1.23 mmol) at 25° C. under N₂. After stirring at 60° C. for 2 hours, the mixture was cooled to 25° C., and a solution of B-1.12 (400 mg, 0.826 mmol) was added slowly. The reaction mixture was stirred for 1.5 hours at 25° C. and then diluted with NH₄Cl (20 mL). The mixture was filtered through a pad of silica gel and the filtrate was extracted with EtOAc (2×20 mL). The organic phase was dried over Na₂SO₄, filtered, and concentrated. The residue was purified by flash column (0-15% of EtOAc in PE) and lyophilized to give B-1.13 (260.7 mg, 82%). ¹H NMR (400 MHz, CDCl₃) δ_{H} 3.67 (s, 3H), 2.42-2.22 (m, 2H), 1.96-1.67 (m, 4H), 1.54-1.24 (m, 14H), 1.20-0.85 (m, 16H), 0.84 (s, 3H), 0.72 (s, 3H), 0.69-0.54 (m, 2H), 0.09-0.01 (m, 1H), LC-ELSD/MS purity >99%, MS ESI calcd. for C₂₉H₄₉O₃ [M−H₂O+H]⁺ 427.4, found 427.4.

### Synthesis of B-1

To a solution of B-1.13 (100 mg, 0.225 mmol) in THF (2 mL) was added MeMgBr (1.49 mL, 3 M, 4.49 mmol) dropwise at 25° C. After stirring at 25° C. for 1 hour under N₂, the reaction mixture was poured into NH₄Cl (10 ml, 10% aq.) and extracted with EtOAc (2×10 mL). The combined organic layer was dried over Na₂SO₄, filtered, and concentrated. Purification by column chromatography (0-10%, EtOAc in PE), trituration with PE, and lyophilization provided B-1 (33.7 mg, 33%). ¹H NMR (400 MHz, CDCl₃) δ_{H} 1.95-1.87 (m, 1H), 1.77-1.71 (m, 1H), 1.64-1.57 (m, 5H), 1.55-1.25 (m, 16H), 1.24-1.22 (m, 6H), 1.20-0.96 (m, 8H), 0.91-0.86 (m, 6H), 0.84 (s, 3H), 0.74-0.66 (m, 4H), 0.61-0.55 (m, 1H), 0.11-0.00 (m, 1H); LC-ELSD/MS purity >99%, MS ESI calcd. for C₃₀H₅₂₀₂ [M−2H₂O+H]⁺ 409.4, found 409.4.

### Example 17: Synthesis of (2S,4aS,4bS,6aR,7S,7aS,8aR,8bS,8cR,10aS)-2-ethyl-4a,6a-dimethyl-7-((2R,5R)-6,6,6-trifluoro-5-hydroxy-5-methylhexan-2-yl)octadecahydrocyclopropa[4,5]cyclopenta[1,2-a]phenanthren-2-ol (B-2) & (2S,4aS,4bS,6aR,7S,7aS,8aR,8bS,8cR,10aS)-2-ethyl-4a,6a-dimethyl-7-((2R,5S)-6,6,6-trifluoro-5-hydroxy-5-methylhexan-2-yl)octadecahydrocyclopropa[4,5]cyclopenta[1,2-a]phenanthren-2-ol (B-3)

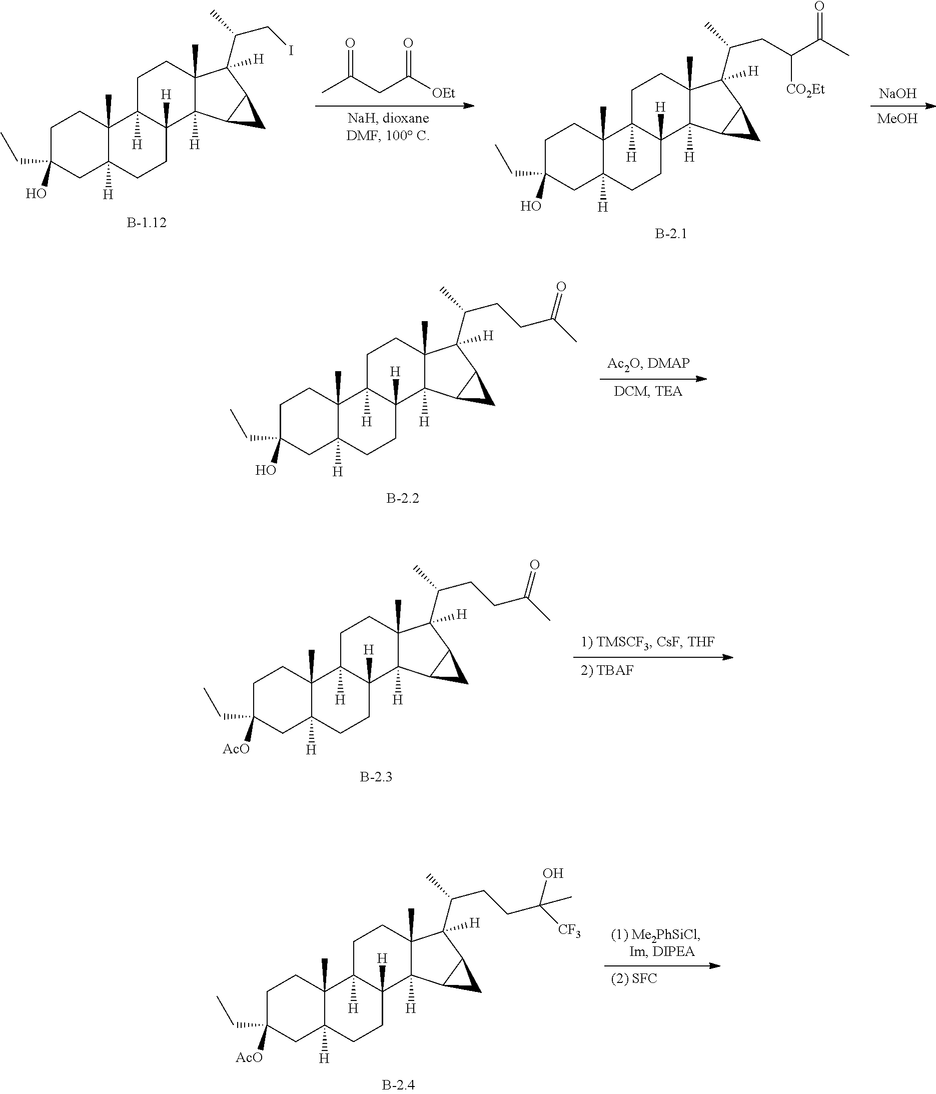

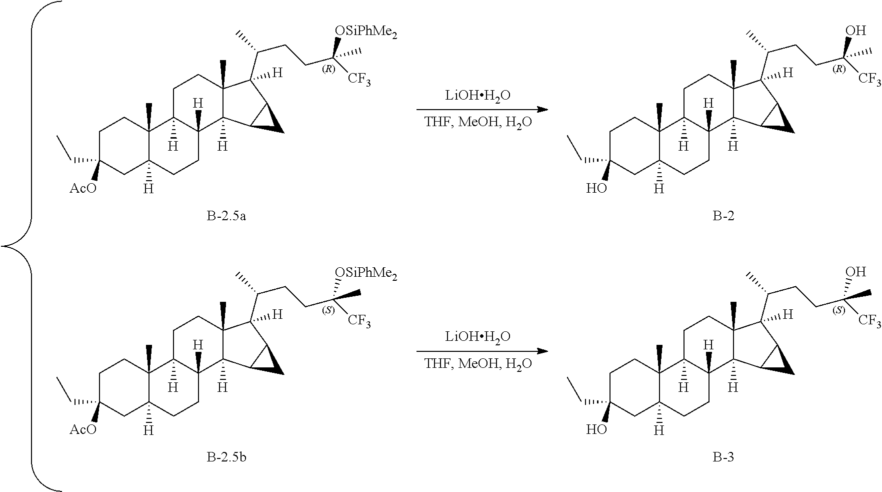

### Synthesis of B-2.1

To a solution of NaH (398 mg, 60%, 10.0 mmol) in dioxane (10 mL) was added ethyl acetoacetate (1.67 g, 12.9 mmol) at 0° C. under N₂. The mixture was stirred at 0° C. for 1 hour. Then DMF (20 mL) was added to the mixture. To the mixture was added a solution of B-1.12 (1.50 g, 3.09 mmol) in dioxane (10 mL) dropwise and the mixture was stirred at 105° C. for 16 hours. The mixture was poured into NH₄Cl (50 mL) and the aqueous phase was extracted with DCM (2×50 mL). The combined organic phase was washed with water (2×50 mL), dried over anhydrous Na₂SO₄, filtered, and concentrated. The residue was purified by flash column chromatography (0˜15% of EtOAc in PE) to give B-2.1 (1.20 g).

### Synthesis of B-2.2

To a mixture of B-2.1 (1.20 g, 2.46 mmol) in MeOH (15 mL) was added H₂O (5 mL) and NaOH (983 mg, 24.6 mmol) at 25° C. The reaction mixture was stirred at 60° C. for 16 hours. The reaction mixture was concentrated and then H₂O (20 mL) was added. The mixture was extracted with EtOAc (3×30 mL). The combined organic phase was washed with brine (15 mL), dried over anhydrous Na₂SO₄, filtered, and concentrated. The residue was purified by silica gel chromatography (20-25% of EtOAc in PE) to give the product B-2.2 (500 mg, 49%). ¹H NMR (400 MHz, CDCl₃) δ_{H} 2.60-2.49 (m, 1H), 2.47-2.37 (m, 1H), 2.16 (s, 3H), 2.03-1.87 (m, 2H), 1.75-1.68 (m, 1H), 1.62-1.60 (m, 1H), 1.57-1.35 (m, 11H), 1.35-0.96 (m, 11H), 0.91-0.85 (m, 6H), 0.84 (s, 3H), 0.72 (s, 3H), 0.70-0.65 (m, 1H), 0.60-0.55 (m, 1H), 0.11-0.03 (m, 1H).

### Synthesis of B-2.3

To a solution of B-2.2 (500 mg, 1.20 mmol) in DCM (5 mL) was added TEA (0.495 mL, 3.59 mmol), DMAP (438 mg, 3.59 mmol) and Ac₂O (245 mg, 2.40 mmol). The mixture was stirred at 25° C. for 16 hours. The mixture was washed with NaHCO₃ (20 mL, sat.), dried over Na₂SO₄, filtered, and concentrated to give B-2.3 (390 mg, 71%). ¹H NMR (400 MHz, CDCl₃) δ_{H} 2.60-2.37 (m, 2H), 2.16 (s, 3H), 2.03-1.98 (m, 3H), 1.97 (s, 3H), 1.96-1.65 (m, 7H), 1.54-1.39 (m, 5H), 1.36-0.92 (m, 11H), 0.90-0.87 (m, 2H), 0.86 (s, 3H), 0.84-0.80 (m, 3H), 0.72 (s, 3H), 0.69-0.62 (m, 1H), 0.60-0.54 (m, 1H), 0.11-0.03 (m, 1H).

### Synthesis of B-2.4

To a solution of B-2.3 (390 mg, 0.854 mmol) in THF (5 mL) was added CsF (517 mg, 3.41 mmol) at 25° C. under N₂. TMSCF₃ (728 mg, 5.12 mmol) was added dropwise at 25° C. The mixture was stirred at 25° C. for 1 hour. To the mixture was added TBAF (5.12 mL, 1 M in THF, 5.12 mmol) and the mixture was stirred at 25° C. for another 16 hours. The mixture was concentrated and the residue was dissolved in EtOAc (10 mL), washed with water (2×10 mL), brine (10 mL), dried over Na₂SO₄, filtered, and concentrated. The residue was purified by flash column chromatography (0˜30% EtOAc in PE) to give B-2.4 (480 mg). ¹H NMR (400 MHz, CDCl₃) δ_{H} 1.97 (s, 3H), 1.94-1.51 (m, 17H), 1.50-1.40 (m, 3H), 1.38-1.34 (m, 3H), 1.31-0.95 (m, 10H), 0.93-0.78 (m, 8H), 0.73 (s, 3H), 0.62-0.54 (m, 1H), 0.12-0.03 (m, 1H).

### Synthesis of B-2.5a & B-2.5b

To a solution of B-2.4 (480 mg, 0.911 mmol) in THF/DMF (6 ml, 1:1) was added 1H-imidazole (987 mg, 14.5 mmol), DIPEA (1.87 g, 14.5 mmol) and Me₂PhSiCl (1.24 g, 7.28 mmol). The reaction mixture was stirred at 25° C. for 36 hours. The mixture was poured into aqueous NaHCO₃ (10 mL) and then extracted with DCM (3×10 mL). The combined organic phase was washed with brine (10 mL), dried over anhydrous Na₂SO₄, filtered, and concentrated. The residue was purified by flash column chromatography (0˜5% of EtOAc in PE) and further purified by SFC (Column: DAICEL CHIRALPAK IG (250 mm*30 mm, 10 um); Condition: 0.1% NH₃H₂O IPA; Begin B: 15%; End B: 15%; Flow Rate (ml/min): 60; Injections: 95) to give B-2.5a (130 mg, 32%) and B-2.5b (140 mg, 35%).

B-2.5a: ¹H NMR (400 MHz, CDCl₃) δ_{H} 7.62-7.55 (m, 2H), 7.40-7.31 (m, 3H), 4.10-3.94 (m, 1H), 2.05-1.99 (m, 2H), 1.97 (s, 3H), 1.96-1.66 (m, 10H), 1.45-1.39 (m, 3H), 1.33 (s, 3H), 1.23-1.18 (m, 6H), 1.14-0.95 (m, 6H), 0.86 (s, 3H), 0.86-0.79 (m, 6H), 0.71 (s, 3H), 0.58-0.52 (m, 1H), 0.47-0.38 (m, 6H), 0.05-0.05 (m, 1H). ¹⁹F NMR (376 MHz, CDCl₃) δ_{F} −81.35 (s).

B-2.5b: ¹H NMR (400 MHz, CDCl₃) δ_{H} 7.61-7.55 (m, 2H), 7.41-7.30 (m, 3H), 4.07-3.98 (m, 1H), 2.05-1.98 (m, 2H), 1.97 (s, 3H), 1.93-1.62 (m, 10H), 1.45-1.39 (m, 2H), 1.33 (s, 3H), 1.31-1.24 (m, 4H), 1.24-1.19 (m, 3H), 1.16-0.94 (m, 6H), 0.86 (s, 3H), 0.86-0.79 (m, 6H), 0.71 (s, 3H), 0.59-0.52 (m, 1H), 0.48-0.37 (m, 6H), 0.05-0.06 (m, 1H). ¹⁹F NMR (376 MHz, CDCl₃) δ_{F} −81.72 (s).

### Synthesis of B-2

To a solution of B-2.5a (130 mg, 0.197 mmol) in THF (6 mL) was added MeOH (3 mL), H₂O (3 mL) and LiOH·H₂O (532 mg, 12.7 mmol) and the mixture was stirred at 25° C. for 18 hours. The mixture was diluted with water (10 mL) and extracted with EtOAc (2×10 mL). The combined organic layer was dried over Na₂SO₄, filtered and concentrated. The residue was purified by flash column chromatography (0˜15% EtOAc in PE) and lyophilized to give B-2 (60.1 mg, 75%). ¹H NMR (400 MHz, CDCl₃) δ_{H} 1.94-1.70 (m, 5H), 1.65-1.56 (m, 7H), 1.54-1.38 (m, 6H), 1.37 (s, 3H), 1.35-0.97 (m, 11H), 0.92-0.86 (m, 6H), 0.84 (s, 3H), 0.73 (s, 3H), 0.71-0.66 (m, 1H), 0.62-0.56 (m, 1H), 0.15-0.03 (m, 1H). ¹⁹F NMR (376 MHz, CDCl₃) δ_{F} −82.82 (s). LC-ELSD/MS purity >99%; MS ESI calcd. for C₂₉H₄₇F₃O₂ [M−H₂O+H]⁺ 467.4, found 467.3.

### Synthesis of B-3

To a solution of B-2.5b (100 mg, 0.151 mmol) in THF (4 mL) was added MeOH (2 mL), H₂O (2 mL) and LiOH·H₂O (440 mg, 10.5 mmol) and the mixture was stirred at 25° C. for 36 hours. The mixture was diluted with water (10 mL) and extracted with EtOAc (2×10 mL). The combined organic layer was dried over Na₂SO₄, filtered, and concentrated. The residue was purified by flash column chromatography (0˜15% EtOAc in PE) and lyophilized to give B-3 (47.9 mg, 50%). ¹H NMR (400 MHz, CDCl₃) δ_{H} 1.95-1.58 (m, 10H), 1.56-1.37 (m, 8H), 1.35 (s, 3H), 1.32-0.96 (m, 11H), 0.92-0.86 (m, 6H), 0.84 (s, 3H), 0.73 (s, 3H), 0.72-0.64 (m, 1H), 0.62-0.56 (m, 1H), 0.12-0.04 (m, 1H). 1° F. NMR (376 MHz, CDCl₃) δ_{F} −83.13 (s). LC-ELSD/MS purity >99%; MS ESI calcd. for C₂₉H₄₇F₃O₂ [M−H₂O+H]⁺ 467.4, found 467.4.

### Example 18: Synthesis of (2S,4aS,4bS,6aR,7S,7aR,8aS,8bS,8cR,10aS)-2-ethyl-7-((R)-6-hydroxy-6-methylheptan-2-yl)-4a,6a-dimethyloctadecahydrocyclopropa[4,5]cyclopenta[1,2-a]phenanthren-2-ol (B-4)

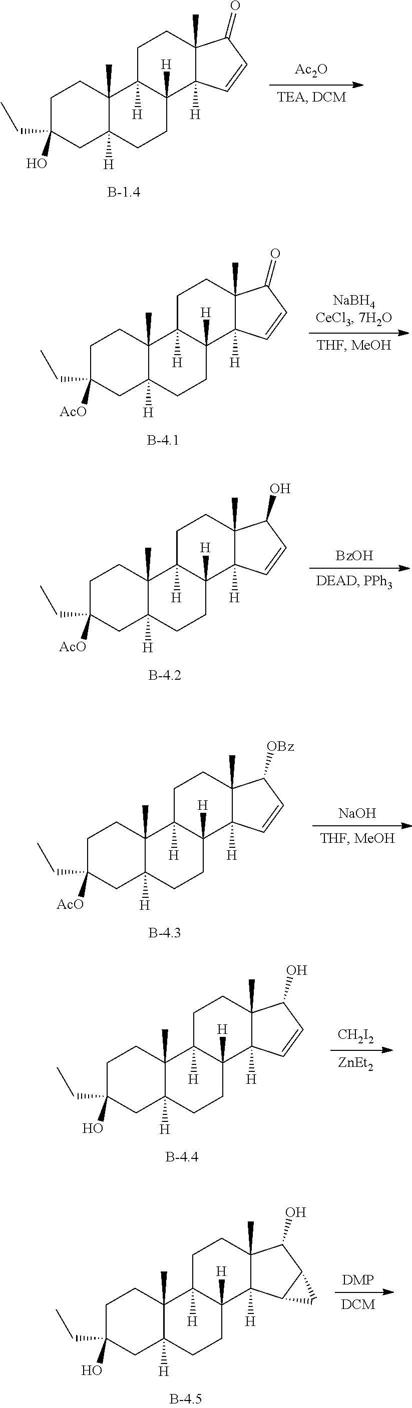

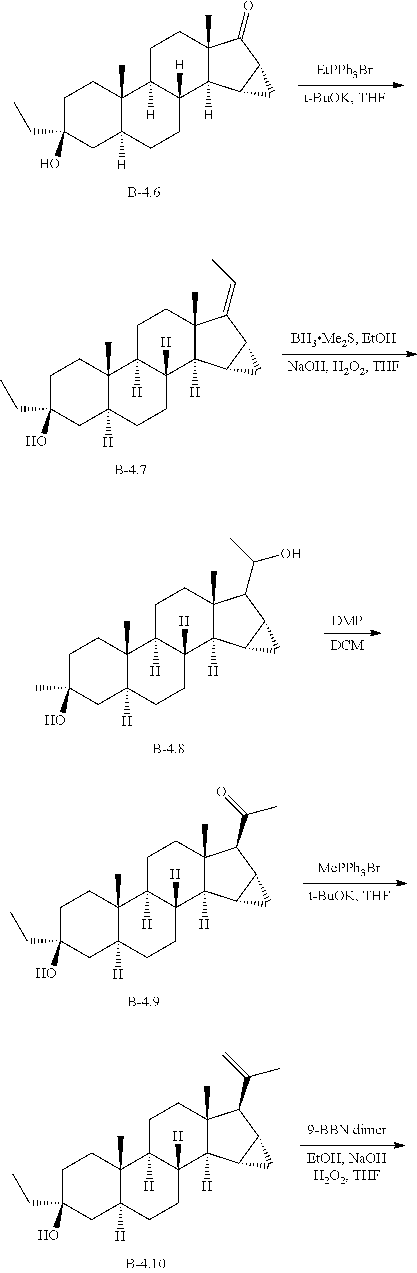

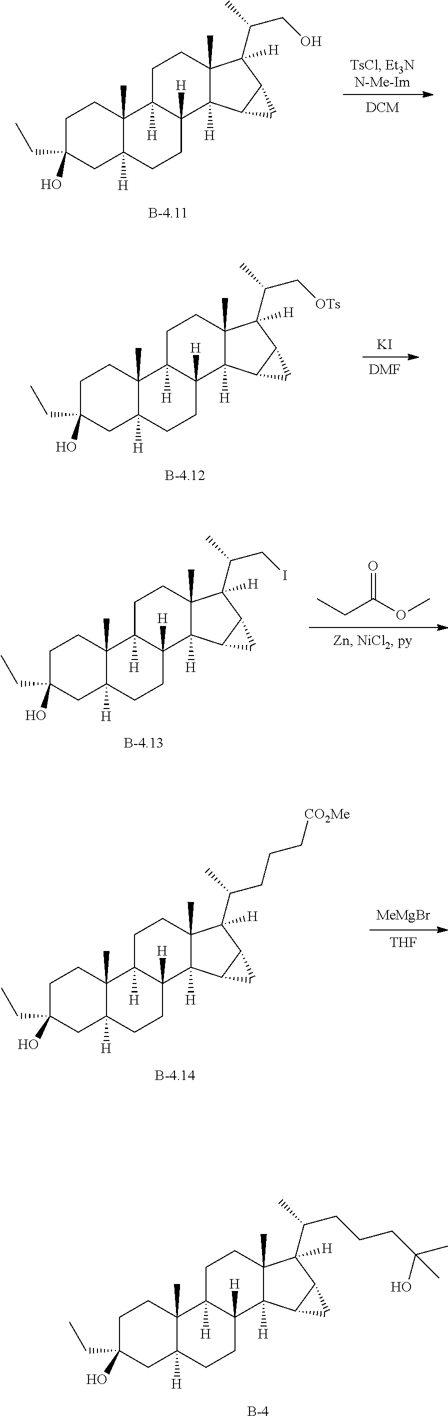

### Synthesis of B-4.1

To a solution of B-1.4 (15.0 g, 47.3 mmol) in DCM (150 mL) was added TEA (14.2 g, 141 mmol), DMAP (5.77 g, 47.3 mmol) and Ac₂O (7.23 g, 70.9 mmol) and the mixture was stirred at 15° C. for 16 hours. The mixture was combined with a second batch and added to NaHCO₃ (500 mL, sat.) and extracted with DCM (2×100 mL). The combined organic layer was washed with brine (2×50 mL), dried over Na₂SO₄, filtered, and concentrated to give B-4.1 (30.0 g). ¹H NMR (400 MHz, CDCl₃) δ_{H} 7.52-7.49 (m, 1H), 6.05-5.95 (m, 1H), 2.28-2.22 (m, 1H), 1.98-1.96 (m, 4H), 1.87-1.73 (m, 6H), 1.71-1.65 (m, 3H), 1.64-1.46 (m, 4H), 1.37-1.25 (m, 3H), 1.04 (s, 3H), 0.92 (s, 3H), 0.78-0.85 (m, 5H).

### Synthesis of B-4.2

To a solution of B-4.1 (15.0 g, 41.8 mmol) in MeOH (150 mL) and THF (30 mL) was added CeCl₃·7H₂O (18.5 g, 50.1 mmol) at 0° C. The reaction mixture was stirred at 0° C. for 10 mins. NaBH₄ (1.90 g, 50.1 mmol) was added and stirred at 0° C. for 1 hour. NH₄Cl (300 mL) was added and then citric acid (100 mL, 20%) was added. The mixture was extracted with DCM (2×150 mL). The combined organic layer was washed with brine (2×100 mL), dried over Na₂SO₄, filtered, and concentrated to give B-4.2 (27.0 g). ¹H NMR (400 MHz, CDCl₃) δ_{H} 5.82-5.98 (m, 1H), 5.72-5.55 (m, 1H), 4.35-4.22 (m, 1H), 1.97-1.95 (m, 4H), 1.88-1.56 (m, 12H), 1.44-1.29 (m, 3H), 1.07-0.93 (m, 3H), 0.87 (s, 3H), 0.84-0.77 (m, 7H), 0.74-0.68 (m, 1H).

### Synthesis of B-4.3

To a solution of B-4.2 (5.80 g, 16.0 mmol) in THF (150 mL) was added Ph₃P (16.8 g, 64.0 mmol) and benzoic acid (7.81 g, 64.0 mmol) at 25° C. DEAD (10.06 mL, 64.0 mmol) in THF (10 mL) was added dropwise at 25° C. and the mixture was stirred for 2 hours. The mixture was combined with another batch from B-4.2 (7.00 g, 19.3 mmol) and added into H₂O (500 mL). The aqueous layer was extracted with DCM (3×500 mL). The combined organic layer was washed with brine (2×200 mL), dried over anhydrous Na₂SO₄, filtered, and concentrated to give B-4.3 (30.0 g).

### Synthesis of B-4.4

To a solution of B-4.3 (38.8 g, 85.6 mmol) in THF (100 ml) and MeOH (250 mL) was added NaOH (33.3 g, 835 mmol, in H₂O (150 mL)) and the mixture was stirred at 25° C. for 16 hours. H₂O (500 mL) was added and THF/MeOH were evaporated, and the mixture was filtered. The filter cake was washed with H₂O (2×50 mL) and concentrated. The residue was purified by silica gel chromatography (0˜100% EtOAc in PE) and triturated with MeOH/H₂O (400 mL, 1:1) at 25° C. to give B-4.4 (20.0 g, 40%). A sample of B-4.4 was purified again to give B-4.4 (56.9 mg). ¹H NMR (400 MHz, CDCl₃) δ_{H} 6.17-6.09 (m, 1H), 5.98-5.91 (m, 1H), 4.11-4.03 (m, 1H), 2.30-2.19 (m, 1H), 1.93-1.83 (m, 1H), 1.71-1.60 (m, 4H), 1.50-1.36 (m, 4H), 1.30-1.24 (m, 4H), 1.21-1.19 (m, 1H), 1.17-1.12 (m, 2H), 1.09-0.96 (m, 3H), 0.91-0.89 (m, 1H), 0.89-0.85 (m, 6H), 0.80 (s, 3H), 0.77-0.71 (m, 1H). LC-ELSD/MS purity >99%, MS ESI calcd. for C₂₁H₃₄O₂ [M−H₂O+H]⁺ 301.3, found 301.1.

### Synthesis of B-4.5

To a solution of B-4.4 (3.00 g, 9.41 mmol) in toluene (10 ml) was added diethylzinc (28.2 mL, 28.2 mmol, 1 M) and diiodomethane (2.27 mL, 28.2 mmol, 3.32 g/mL) at 0° C. for 30 mins. Then the mixture was stirred 25° C. for 16 hours. The mixture was quenched with NH₄Cl (150 mL) and extracted with DCM (3×150 mL). The combined organic layer was washed with brine (2×100 mL), dried over anhydrous sodium sulfate, filtered, and concentrated. The residue was triturated with MeOH/H₂O (200 mL, 1:1) to provide B-4.5 (2.70 g, 87%). B-4.5 was combined with a second batch (10.3 g) and triturated with MeOH/H₂O (300 mL, 1:1) to give B-4.5 (8.6 g). B-4.5 (100 mg) was purified from MeCN (5 mL) to give B-4.5 (42.3 mg, 42%). ¹H NMR (400 MHz, CDCl₃) δ_{H} 3.94-3.82 (m, 1H), 1.99-1.91 (m, 1H), 1.71-1.60 (m, 2H), 1.54-1.49 (m, 3H), 1.42-1.34 (m, 4H), 1.32-1.27 (m, 2H), 1.26-1.21 (m, 3H), 1.22-1.18 (m, 2H), 1.13-0.95 (m, 4H), 0.92-0.81 (m, 12H), 0.80-0.76 (m, 1H), 0.63-0.54 (m, 1H). LC-ELSD/MS purity >99%, MS ESI calcd. for C₂₂H₃₆O₂ [M−H₂O+H]⁺ 315.3, found 315.3.

### Synthesis of B-4.6

To a solution of B-4.5 (4.00 g, 12.0 mmol) in DCM (40 mL) was added DMP (10.1 g, 24.0 mmol) at 25° C. and the reaction mixture was stirred at 25° C. for 10 mins. The mixture was quenched with saturated NaHCO₃/Na₂S₂O₃ (1:1, 200 mL) at 10° C. The reaction mixture (combined with another batch) was extracted with DCM (2×200 mL). The combined organic layer was washed with brine (2×100 mL), dried over Na₂SO₄, filtered, and concentrated to give B-4.6 (9.00 g). A sample was purified by flash column chromatography (0˜30% of EtOAc in PE) to give B-4.6 (42.3 mg). ¹H NMR (400 MHz, CDCl₃) δ_{H} 2.08-1.98 (m, 1H), 1.90-1.81 (m, 1H), 1.78-1.57 (m, 6H), 1.55-1.14 (m, 12H), 1.13-1.10 (m, 3H), 1.10-0.94 (m, 2H), 0.93-0.85 (m, 6H), 0.84-0.77 (m, 2H), 0.69-0.60 (m, 1H). LC-ELSD/MS purity >99%, MS ESI calcd. for C22H₃₄₀₂ [M−H₂O+H]⁺ 313.3, found 313.3.

### Synthesis of B-4.7

To a mixture of EtPPh₃Br (26.5 g, 71.6 mmol) in THF (120 mL) was added t-BuOK (8.02 g, 71.6 mmol) at 20° C. under N₂. The resulting mixture was stirred at 50° C. for 30 mins. B-4.6 (7.90 g, 23.9 mmol) in THF (80 mL) was added in portions below 50° C. The reaction mixture was stirred at 50° C. for 16 hours. The reaction mixture was quenched with 10% NH₄Cl aqueous (300 mL) at 15° C. The aqueous was extracted with EtOAc (3×200 mL). The combined organic phase was washed with brine (2×150 mL), dried over anhydrous Na₂SO₄, filtered, and concentrated. The residue was triturated with MeOH/H₂O (450 mL, 1:1) to give B-4.7 (9.70 g). ¹H NMR (400 MHz, CDCl₃) δ_{H} 5.30-5.25 (m, 1H), 2.16-2.05 (m, 1H), 1.99-1.91 (m, 1H), 1.76-1.67 (m, 2H), 1.65-1.62 (m, 4H), 1.47-1.36 (m, 4H), 1.27-1.25 (m, 3H), 1.18-1.12 (m, 3H), 1.08-1.05 (m, 3H), 1.04-0.94 (m, 4H), 0.91-0.83 (m, 9H), 0.71-0.66 (m, 1H), 0.64-0.57 (m, 1H), 0.36-0.29 (m, 1H).

### Synthesis of B-4.8

To a solution of B-4.7 (9.70 g, 28.3 mmol) in THF (100 mL) was added BH₃·Me₂S (5.66 mL, 10M, 56.6 mmol) under N₂ and the reaction mixture was stirred at 20° C. under N₂ for 12 hours. The mixture was cooled to 0° C. EtOH (19.8 mL, 339 mmol) and NaOH (67.8 mL, 5M, 339 mmol) were added to the mixture and then H₂O₂ (33.9 mL, 10M, 339 mmol) was added dropwise at 15° C. The mixture was stirred at 70° C. for 1 hour. Then the mixture was poured into Na₂SO₃ (200 mL, sat.) and extracted with EtOAc (3×200 mL). The combined organic layer was washed with brine (2×150 mL), dried over anhydrous Na₂SO₄, filtered, and concentrated. The residue was purified by flash column chromatography (0˜70% ethyl acetate in PE) to give B-4.8 (7.60 g, 75%). ¹H NMR (400 MHz, CDCl₃) δ_{H} 3.98-3.73 (m, 1H), 1.97-1.89 (m, 1H), 1.72-1.64 (m, 1H), 1.54-1.45 (m, 3H), 1.41-1.36 (m, 2H), 1.34-1.24 (m, 5H), 1.22-1.19 (m, 4H), 1.17-0.91 (m, 10H), 0.91-0.82 (m, 10H), 0.62-0.51 (m, 2H), 0.42-0.36 (m, 1H).

### Synthesis of B-4.9

To a solution of B-4.8 (5.00 g, 13.8 mmol) in DCM (50 mL) was added DMP (11.7 g, 27.6 mmol) at 25° C. and the reaction mixture was stirred for 10 mins. The mixture was quenched by saturated NaHCO₃/Na₂S₂O₃ (1:1 200 mL) and extracted with DCM (3×150 mL). The combined organic layer was washed with brine (2×100 mL), dried over Na₂SO₄, filtered, and concentrated. The residue was purified by flash column chromatography (0˜30% ethyl acetate in PE) to give B-4.9 (2.60 g, 53%). ¹H NMR (400 MHz, CDCl₃) δ_{H} 2.17-2.06 (m, 4H), 1.98-1.90 (m, 1H), 1.87-1.78 (m, 2H), 1.53-1.48 (m, 3H), 1.46-1.19 (m, 10H), 1.18-0.91 (m, 6H), 0.90-0.86 (m, 3H), 0.85-0.80 (m, 6H), 0.68-0.59 (m, 2H), 0.39-0.33 (m, 1H). LC-ELSD/MS purity >99%, MS ESI calcd. for C₂₄H₃₈O₂ [M−H₂O+H]⁺ 341.3, found 341.3.

### Synthesis of B-4.10

To a mixture of MePPh₃Br (14.9 g, 41.8 mmol) in THF (60 mL) was added t-BuOK (4.69 g, 41.8 mmol) at 25° C. under N₂. The resulting mixture was stirred at 55° C. for 30 mins. B-4.9 (3.00 g, 8.36 mmol) in THF (30 mL) was added in portions below 50° C. The reaction mixture was stirred at 55° C. for 16 hours. The reaction mixture was quenched with 10% NH₄Cl aqueous (100 mL) and extracted with EtOAc (3×100 mL). The combined organic layer was washed with brine (2×50 mL), dried over Na₂SO₄, filtered, and concentrated. The residue was triturated with MeOH/H₂O (300 mL, 1:1) to give B-4.10 (3.00 g). ¹H NMR (400 MHz, CDCl₃) δ_{H} 4.90-4.85 (m, 1H), 4.81-4.75 (m, 1H), 1.98-1.92 (m, 1H), 1.88-1.83 (m, 1H), 1.76 (s, 3H), 1.54-1.35 (m, 9H), 1.31-1.24 (m, 4H), 1.16-1.04 (m, 5H), 1.01-0.93 (m, 2H), 0.91-0.86 (m, 4H), 0.84 (s, 3H), 0.81 (s, 3H), 0.63-0.57 (m, 2H), 0.43-0.36 (m, 1H).

### Synthesis of B-4.11

To a solution of B-4.10 (3.00 g, 8.41 mmol) in THF (30 mL) was added 9-BBN dimer (6.14 g, 25.2 mmol) at 25° C. under N₂ then the reaction was stirred at 45° C. for 16 hours. To the resulting mixture was added ethanol (8.79 mL, 151 mmol) at 25° C., followed by NaOH (30.1 mL, 5.0 M, 151 mmol) at 0° C. Hydrogen peroxide (15.0 mL, 10 M, 151 mmol) was added dropwise at 0° C. and the reaction mixture was stirred at 70° C. for 1 hour. The mixture was cooled to 25° C. and saturated Na₂SO₃ (100 mL) was added. The aqueous layer was extracted with EtOAc (3×100 mL) and the combined organic layer was washed with brine (2×50 mL), dried over Na₂SO₄, filtered, and concentrated. The residue was purified by flash column (0˜35% of EtOAc in PE) to give B-4.11 (2.80 g). ¹H NMR (400 MHz, CDCl₃) δ_{H} 3.85-3.79 (m, 1H), 3.54-3.47 (m, 1H), 1.95-1.90 (m, 1H), 1.80-1.70 (m, 2H), 1.43-1.06 (m, 16H), 1.02-0.97 (m, 6H), 0.95-0.91 (m, 4H), 0.90-0.84 (m, 7H), 0.73-0.68 (m, 1H), 0.61-0.49 (m, 2H), 0.34-0.29 (m, 1H).

### Synthesis of B-4.12

To a solution of B-4.11 (2.80 g, 7.47 mmol) in DCM (45 mL) was added N-Me-Im (2.44 g, 29.8 mmol), TEA (755 mg, 7.47 mmol), and TsCl (2.13 g, 11.2 mmol) and the mixture was stirred at 20° C. for 30 mins. Water (100 mL) added and the mixture was stirred for 30 mins then extracted with EtOAc (3×100 mL). The combined organic layer was washed with brine (2×50 mL), dried over Na₂SO₄, filtered, and concentrated. The residue was purified by flash column chromatography (0˜30% of EtOAc in PE) and further purified by SFC (column: DAICEL CHIRALPAK AD (250 mm*50 mm, 10 um); condition: 0.1% NH₃H₂O ETOH; gradient: from 45% to 45% of B, FlowRate (ml/min): 220) to give B-4.12 (2.30 g, 70%). ¹H NMR (400 MHz, CDCl₃) δ_{H} 7.84-7.77 (m, 2H), 7.37-7.31 (m, 2H), 4.27-4.20 (m, 1H), 3.88-3.81 (m, 1H), 2.45 (s, 3H), 1.92-1.83 (m, 3H), 1.75-1.68 (m, 1H), 1.50-0.99 (m, 15H), 0.97-0.92 (m, 5H), 0.90-0.85 (m, 7H), 0.82 (s, 3H), 0.80-0.75 (m, 1H), 0.71-0.65 (m, 1H), 0.59-0.44 (m, 2H), 0.25-0.19 (m, 1H).

### Synthesis of B-4.13

To a solution of B-4.12 (2.30 g, 4.34 mmol) in DMF (30 mL) was added KI (3.60 g, 21.7 mmol) at 25° C. and the mixture was stirred at 60° C. for 1 hour. The mixture was poured into water (100 mL) and stirred for 20 mins. The aqueous phase was extracted with EtOAc (3×100 mL). The combined organic phase was washed with brine (2×50 mL), dried over anhydrous Na₂SO₄, filtered, and concentrated. The residue was purified by flash column chromatography (0-30% of EtOAc in PE) to give B-4.13 (2.60 g). ¹H NMR (400 MHz, CDCl₃) δ_{H} 3.60-3.50 (m, 1H), 3.30-3.16 (m, 1H), 1.97-1.89 (m, 1H), 1.78-1.70 (m, 1H), 1.56-1.20 (m, 14H), 1.19-1.02 (m, 4H), 1.01-0.95 (m, 5H), 0.93 (s, 3H), 0.91-0.85 (m, 4H), 0.84 (s, 3H), 0.77-0.69 (m, 1H), 0.60-0.51 (m, 2H), 0.43-0.30 (m, 1H).

### Synthesis of B-4.14

To a mixture of Zn powder (188 mg, 2.88 mmol) in dry pyridine (2 mL) was added slowly methyl acrylate (247 mg, 2.88 mmol) and NiCl₂ (80.2 mg, 0.62 mmol) at 20° C. under N₂. After stirring at 60° C. for 2 hours, the mixture was cooled to 20° C. and a solution of B-4.13 (200 mg, 0.413 mmol) in pyridine (2 mL) was added to the mixture slowly. The reaction mixture was stirred for 1.5 hours at 20° C. The mixture was quenched with NH₄Cl (50 mL) and filtered through a pad of silica gel. The filtrate was extracted with EtOAc (2×50 mL) and the organic phase was dried over Na₂SO₄, filtered, and concentrated. The residue was purified by flash column chromatography (0-20% of EtOAc in PE) to give B-4.14 (65 mg, 36%). ¹H NMR (400 MHz, CDCl₃) δ_{H} 3.67 (s, 3H), 2.39-2.23 (m, 2H), 1.96-1.87 (m, 1H), 1.84-1.64 (m, 4H), 1.51-0.94 (m, 21H), 0.91-0.82 (m, 13H), 0.68-0.63 (m, 1H), 0.59-0.47 (m, 2H), 0.32-0.25 (m, 1H). LC-ELSD/MS purity >99%, MS ESI calcd. for C₂₉H₄₈₀₃ [M−H₂O+H]⁺ 427.3, found 427.3.

### Synthesis of B-4

A solution of MeMgBr (0.44 mL, 1.34 mmol, 3 M) in THF (1 ml) was reacted with B-4.14 (60.0 mg, 0.135 mmol) at 0° C. under N₂ and the reaction mixture was stirred at 20° C. for 2 hours. The reaction mixture was quenched by saturated NH₄Cl (30 mL) and extracted with ethyl acetate (3×30 mL). The combined organic phase was washed with brine (2×30 mL), dried over anhydrous Na₂SO₄, filtered, and concentrated. The residue was purified by flash column chromatography (0˜25% of EtOAc in PE) to give B-4 (30.9 mg, 52%). 1H NMR (400 MHz, CDCl₃) δ_{H} 1.97-1.88 (m, 1H), 1.82-1.74 (m, 1H), 1.69-1.60 (m, 2H), 1.56-1.25 (m, 15H), 1.23-1.18 (m, 8H), 1.17-0.92 (m, 8H), 0.92-0.82 (m, 13H), 0.69-0.46 (m, 3H), 0.31-0.24 (m, 1H). LC-ELSD/MS purity >99%, MS ESI calcd. for C₃₀H₅₂O₂ [M−2H₂O+H]⁺ 409.4, found 409.4.

### Example 19: Synthesis of (2S,4aS,4bS,6aR,7S,7aR,8aS,8bS,8cR,10aS)-2-ethyl-4a,6a-dimethyl-7-((2R,5S)-6,6,6-trifluoro-5-hydroxy-5-methylhexan-2-yl)octadecahydrocyclopropa[4,5]cyclopenta[1,2-a]phenanthren-2-ol (B-5)

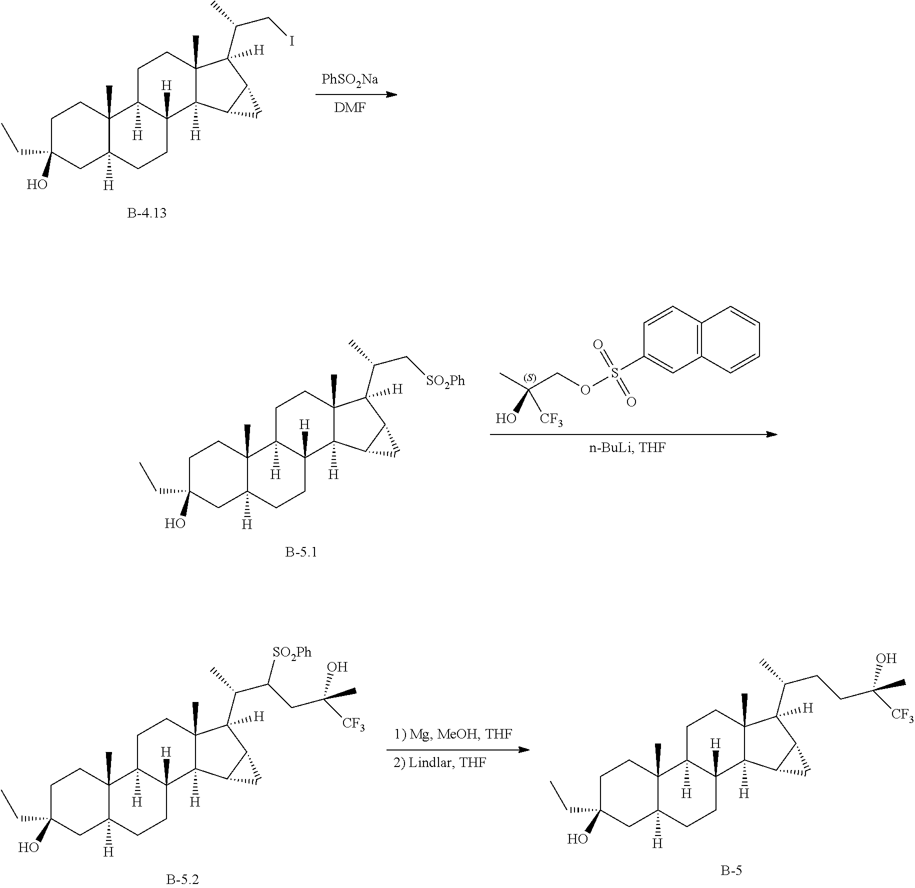

### Synthesis of B-5.1

To a solution of B-4.13 (1.50 g, 3.09 mmol) in DMF (15 mL) was added PhSO₂Na (1.52 g, 9.27 mmol) and the mixture was stirred at 40° C. for 16 hours. The reaction mixture was poured into water (100 mL) and stirred 10 mins. The mixture was filtered and purified by flash column chromatography (15˜25% EtOAc in PE) to give B-5.1 (2.00 g). ¹H NMR (400 MHz, CDCl₃) δ_{H} 7.97-7.90 (m, 2H), 7.69-7.62 (m, 1H), 7.60-7.53 (m, 2H), 3.52-3.40 (m, 1H), 3.00-2.87 (m, 1H), 2.32-2.19 (m, 1H), 1.92-1.83 (m, 1H), 1.77-1.69 (m, 1H), 1.52-1.16 (m, 12H), 1.14-1.08 (m, 4H), 1.07-0.92 (m, 5H), 0.91-0.81 (m, 10H), 0.76-0.67 (m, 2H), 0.57-0.45 (m, 2H), 0.30-0.21 (m, 1H).

### Synthesis of B-5.2

To a solution of B-5.1 (430 mg, 0.862 mmol) in THF (10 mL) was added n-BuLi (1.37 mL, 3.44 mmol, 2.5M in hexane) dropwise at -70° C. under N₂. The reaction mixture was stirred at -70° C. for 0.5 hour. DIPA (483 μL, 3.44 mmol) was added dropwise at −70° C. for 0.5 hour. A solution of (2S)-1,1,1-trifluoro-2-methyl-3-[(naphthalene-2-sulfonyl)oxy]propan-2-ol (374 mg, 1.12 mmol) in THF (2 ml) was added dropwise at −70° C. The reaction mixture was warmed to 25° C. slowly and stirred at 25° C. for 18 hours. The reaction mixture was quenched with saturated NH₄Cl (50 mL) and extracted with EtOAc (3×50 mL). The combined organic phase was washed with brine (2×30 mL), dried over Na₂SO₄, filtered, and concentrated to give B-5.2 (540 mg). ¹H NMR (400 MHz, CDCl₃) δ_{H} 8.02-7.87 (m, 2H), 7.77-7.50 (m, 3H), 3.72-3.40 (m, 1H), 3.00-2.50 (m, 1H), 2.31-1.65 (m, 4H), 1.53-1.16 (m, 14H), 1.13-1.05 (m, 4H), 1.00-0.78 (m, 15H), 0.74-0.41 (m, 5H), 0.30-0.05 (m, 2H). ¹⁹F NMR (376 MHz, CDCl₃) δ_{F} −83.69 (s).

### Synthesis of B-5

To a solution of B-5.2 (340 mg, 0.544 mmol) in methanol (12 mL) and THF (3 mL) was added nickel (II) chloride (13.9 mg, 0.108 mmol) and Mg powder (19.6 g, 820 mmol) in four portions under N₂ at 65° C. The reaction mixture was stirred at 65° C. for 1 hour. The reaction mixture was added dropwise to citric acid (25 mL) and extracted with EtOAc (3×20 mL). The combined organic phase was washed with brine (2×15 mL), dried over anhydrous Na₂SO₄, filtered and concentrated. The residue was purified by flash column chromatography (0˜40% of EtOAc in PE) to give 170 mg of product. To the product in THF (5 mL) was added Lindlar (100 mg). The suspension was degassed under vacuum and purged with H₂ three times. The mixture was stirred under H₂ (15 psi) at 20° C. for 16 hours then the reaction mixture was filtered through a pad of Celite and washed with THF (3×10 mL). The filtrate was concentrated and purified by flash column chromatography (0˜20% EtOAc in PE) to provide B-5 (56.6 mg, 44%). ¹H NMR (400 MHz, CDCl₃) δ_{H} 1.96-1.89 (m, 1H), 1.88-1.56 (m, 9H), 1.54-1.37 (m, 5H), 1.35 (s, 3H), 1.34-0.93 (m, 13H), 0.92-0.82 (m, 12H), 0.72-0.66 (m, 1H), 0.61-0.48 (m, 2H), 0.33-0.26 (m, 1H). LC-ELSD/MS purity >99%, MS ESI calcd. for C₂₉H₄₇F₃O₂ [M−H₂O+H]⁺ 467.3, found 467.4. ¹⁹F NMR (376 MHz, CDCl₃) δ_{F} −83.20 (s).

### Example 20: Synthesis of (2S,4aS,4bS,6aR,7S,7aR,8aS,8bS,8cR,10aS)-2-ethyl-7-((R)-5-hydroxy-5-methylhexan-2-yl)-4a,6a-dimethyloctadecahydrocyclopropa[4,5[cyclopenta[],2-a]phenanthren-2-ol (B-6)

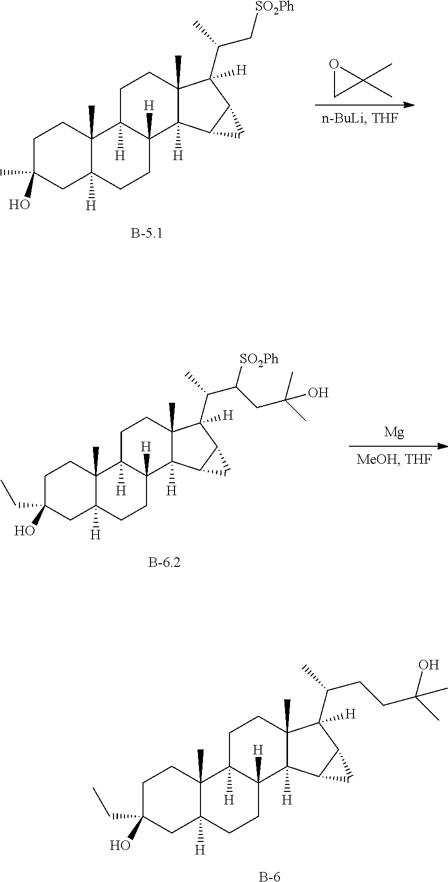

### Synthesis of B-6.2

To a solution of B-5.1 (1.00 g, 2.00 mmol) in THF (10 mL) was added n-BuLi (3.20 mL, 8.00 mmol, 2.5M in hexane) dropwise at -70° C. under N₂. The reaction mixture was stirred at −70° C. for 0.5 hour. DIPA (1.12 mL, 8.00 mmol) was added dropwise at −70° C. for 0.5 hour. A solution of 2,2-dimethyloxirane (187 mg, 2.60 mmol) in THF (2 ml) was added dropwise at −70° C. The reaction mixture was warmed to 25° C. slowly and stirred at 25° C. for 18 hours. The reaction mixture was quenched with saturated NH₄Cl (100 mL) and extracted with EtOAc (3×100 mL). The combined organic phase was washed with brine (2×50 mL), dried over Na₂SO₄, filtered, and concentrated to give B-6.2 (1.00 g). ¹H NMR (400 MHz, CDCl₃) δ_{H} 7.99-7.91 (m, 2H), 7.72-7.53 (m, 3H), 3.92-3.80 (m, 1H), 2.37-1.89 (m, 4H), 1.39-1.36 (m, 3H), 1.27-1.23 (m, 7H), 1.21-1.26 (m, 3H), 0.99-0.78 (m, 24H), 0.73-0.68 (m, 2H), 0.61-0.36 (m, 3H), 0.25-0.19 (m, 1H), 0.11-0.05 (m, 1H).

### Synthesis of B-6

To a solution of B-6.2 (1.00 g, 1.75 mmol) in methanol (20 mL) and THF (5 mL) was added nickel (II) chloride (45.3 mg, 0.35 mmol) and Mg powder (1.68 g, 70.0 mmol) in four portions under N₂ at 65° C. and the reaction mixture was stirred at 65° C. for 1 hour. The reaction mixture was added dropwise to citric acid (100 mL) and extracted with EtOAc (3×100 mL). The combined organic phase was washed with brine (2×50 mL), dried over anhydrous Na₂SO₄, filtered, and concentrated. The residue was purified by flash column (0˜40% of EtOAc in PE) and purified from MeCN (10 mL) to give B-6 (101.9 mg, 14%). 1H NMR (400 MHz, CDCl₃) δ_{H} 1.96-1.87 (m, 1H), 1.80-1.59 (m, 4H), 1.54-1.25 (m, 14H), 1.23-1.21 (m, 6H), 1.18-0.93 (m, 8H), 0.92-0.82 (m, 13H), 0.72-0.66 (m, 1H), 0.60-0.49 (m, 2H), 0.32-0.25 (m, 1H). LC-ELSD/MS purity >99%, MS ESI calcd. for C₂₉H₅₀₀₂ [M−2H₂O+H]⁺ 395.4, found 395.3.

### Example 21: Synthesis of (2S,4aS,4bS,6aR,7S,7aR,8aS,8bS,8cR,10aS)-2-ethyl-4a, 6a-dimethyl-7-((2R,5R)-6,6,6-trifluoro-5-hydroxy-5-methylhexan-2-yl)octadecahydrocyclopropa[4,5]cyclopenta[1,2-a]phenanthren-2-ol (B-7)

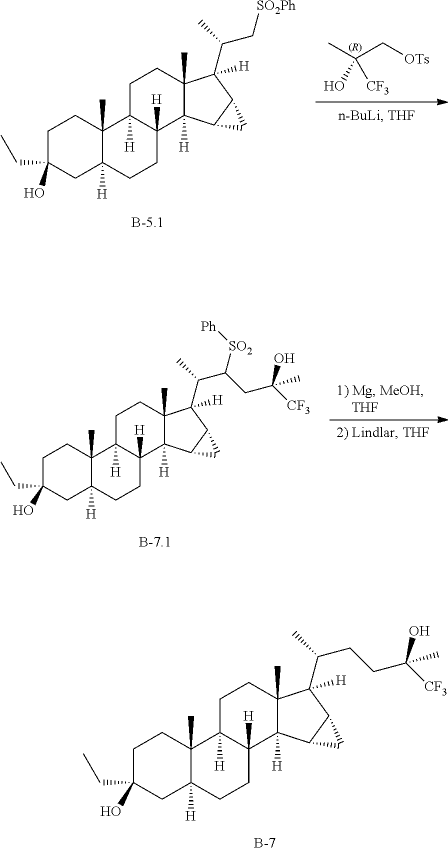

### Synthesis of B-7.1

To a solution of B-5.1 (1.00 g, 2.00 mmol) in THF (10 mL) was added n-BuLi (3.20 mL, 8.00 mmol, 2.5M in hexane) dropwise at −70° C. under N₂. The reaction mixture was stirred at -70° C. for 0.5 hour. Then DIPA (1.12 mL, 8.00 mmol) was added dropwise at −70° C. for 0.5 hour. A solution of (2R)-1,1,1-trifluoro-2-methyl-3-[(4-methylbenzenesulfonyl)oxy]propan-2-ol (775 mg, 2.60 mmol) in THF (5 ml) was added dropwise at −70° C. The reaction mixture was warmed to 25° C. slowly and stirred for 18 hours. The reaction mixture was quenched with saturated NH₄Cl (100 mL) and extracted with EtOAc (3×100 mL). The combined organic phase was washed with brine (2×50 mL), dried over Na₂SO₄, filtered, and concentrated to give B-7.1 (800 mg). ¹⁹F NMR (376 MHz, CDCl₃) δ_{F} −79.90 (s), −83.62 (s).

### Synthesis of B-7

To a solution of B-7.1 (800 mg, 1.28 mmol) in methanol (20 mL) and THF (5 mL) was added nickel (II) chloride (33.1 mg, 256 umol) and Mg powder (1.22 g, 51.2 mmol) in four portions under N₂ at 65° C. The reaction mixture was stirred at 65° C. for 1 hour. The reaction mixture was added dropwise to citric acid (100 mL) and extracted with EtOAc (3×100 mL). The combined organic phase was washed with brine (2×50 mL), dried over anhydrous Na₂SO₄, filtered, and concentrated. The residue was purified by flash column chromatography (0˜40% of EtOAc in PE) to give 300 mg of the product. To a mixture of the product in THF (5 mL) was added Lindlar (300 mg, 10%). The suspension was degassed under vacuum and purged with H₂ three times. The mixture was stirred under H₂ (15 psi) at 20° C. for 16 hours then filtered through a pad of Celite and washed with THF (3×10 mL) and concentrated. The residue was purified from MeCN (10 mL) to give B-7 (135 mg, 45%). 1H NMR (400 MHz, CDCl₃) δ_{H} 1.98-1.68 (m, 5H), 1.57-1.33 (m, 15H), 1.31-0.92 (m, 11H), 0.91-0.82 (m, 12H), 0.74-0.67 (m, 1H), 0.61-0.49 (m, 2H), 0.35-0.27 (m, 1H). ¹⁹F NMR (376 MHz, CDCl₃) δ_{F} −82.89 (s). LC-ELSD/MS purity >99%, MS ESI calcd. for C₂₉H₄₇F₃O₂ [M−H₂O+H]⁺ 467.3, found 467.3.

### Example 22: Synthesis of (2aS,4S,6aS,6bS,8aS,8bS,9aS,10aS,10bR)-4-ethyl-8b-((R)-6-hydroxy-6-methylheptan-2-yl)-6a,8a-dimethyloctadecahydrocyclopropa[3,4]cyclopenta[1,2-a]phenanthren-4-ol (B-8)

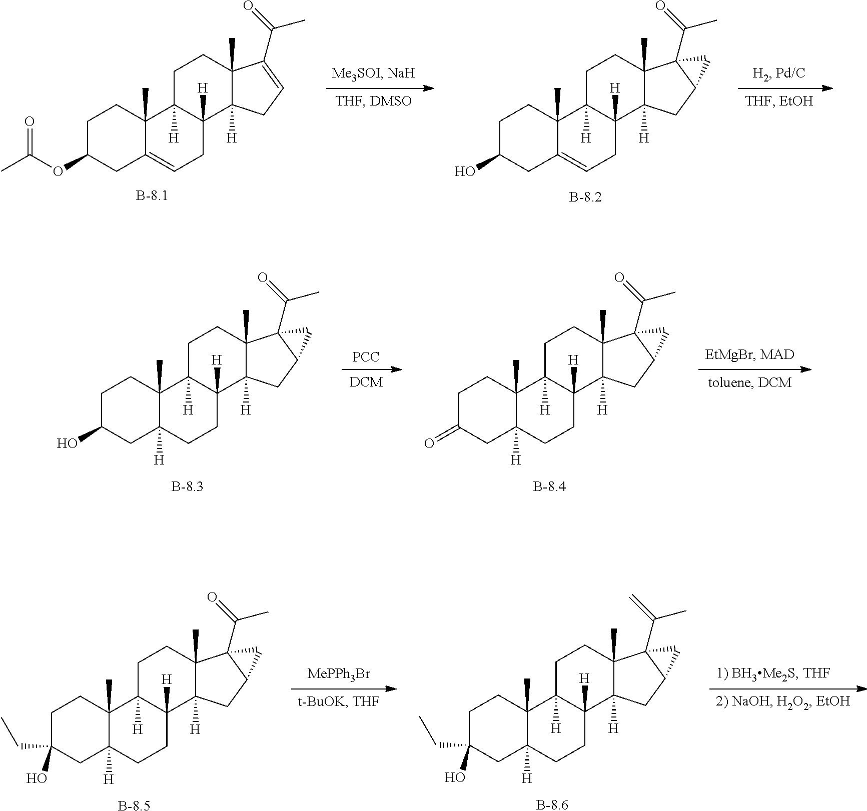

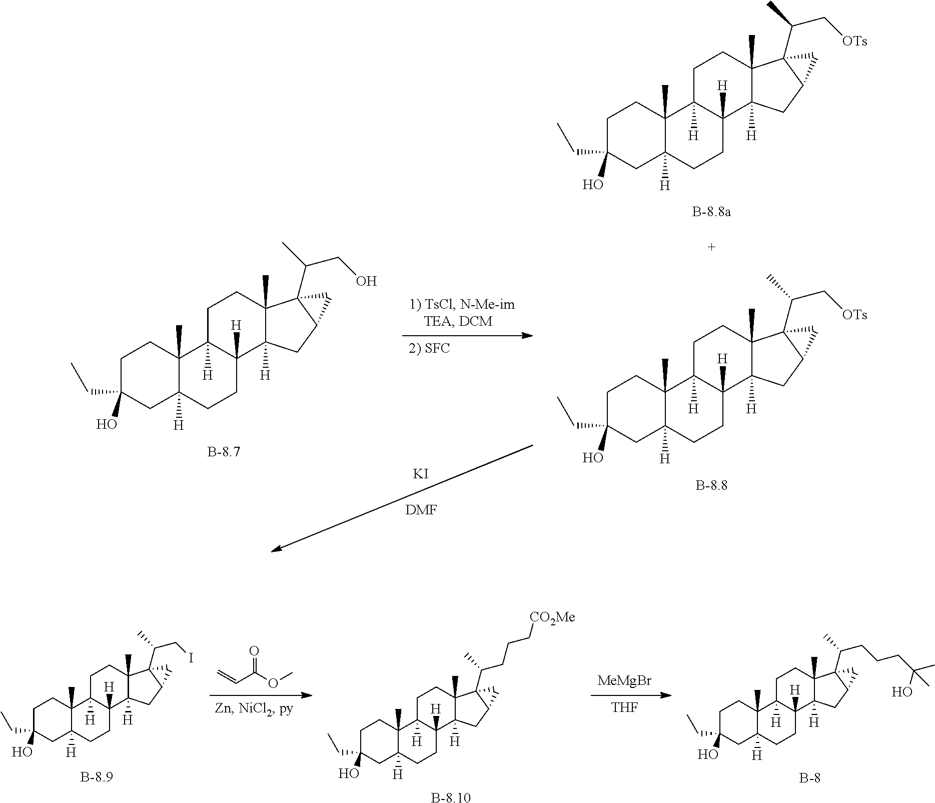

### Synthesis of B-8.2

To a mixture of Me₃SOI (37.7 g, 126 mmol) in DMSO (300 mL) and THF (120 mL) was added NaH (6.71 g, 168 mmol, 60%) at 20° C. and the mixture was stirred for 1 hour. Then a solution of B-8.1 (30.0 g, 84.1 mmol) in THF (40 mL) was added and the mixture was stirred at 20° C. for 24 hours. The mixture was poured into water (1000 mL) and the aqueous phase was extracted with DCM (3×400 mL). The combined organic phase was washed with brine (300 mL), dried over anhydrous Na₂SO₄, filtered, and concentrated. The residue was dissolved in MeOH (500 mL) and THF (500 mL) and K₂CO₃ (11.6 g, 84.1 mmol) was added. The mixture was stirred at 20° C. for 12 hours then poured into H₂O (500 mL) and stirred 15 mins. The mixture was filtered and the filter cake was dissolved in DCM (500 mL). The organic layer was dried over Na₂SO₄, filtrated, and concentrated. The residue was triturated with MeOH (200 mL) to give B-8.2 (24.3 g, 88%). ¹HNMR (400 MHz, CDCl₃) δ_{H} 5.35-5.31 (m, 1H), 3.56-3.44 (m, 1H), 2.35-2.17 (m, 3H), 1.95 (s, 3H), 1.93-1.79 (m, 4H), 1.64-1.51 (m, 5H), 1.44-1.21 (m, 5H), 1.02 (s, 3H), 0.97 (s, 3H), 0.95-0.73 (m, 4H).

### Synthesis of B-8.3

To a solution of B-8.2 (24.0 g, 73.0 mmol) in THF (250 mL) and EtOH (250 mL) was added Pd—C(dry, 10%, 3.00 g) under Ar. The suspension was degassed under vacuum and purged with H₂ three times. The mixture was stirred under H₂ (30 psi) at 25° C. for 24 hours then filtered through a pad of Celite and washed with THF (3×100 mL). The filtrate was concentrated to give B-8.3 (30.0 g). ¹H NMR (400 MHz, CDCl₃) δ_{H} 3.64-3.50 (m, 1H), 2.28-2.17 (m, 1H), 1.94 (s, 3H), 1.90-1.67 (m, 3H), 1.50-1.22 (m, 11H), 1.13-0.92 (m, 6H), 0.90-0.60 (m, 9H).

### Synthesis of B-8.4

To a mixture of B-8.3 (30.0 g, 90.7 mmol) in DCM (450 mL) was added silica gel (29.3 g) and PCC (29.3 g, 136 mmol) at 0° C. The reaction mixture was stirred at 25° C. for 1 hour followed by addition of PE (250 mL). The mixture was filtered, washed with DCM (5×100 mL), and the solvents were concentrated to provide B-8.4 (24.5 g, 82%). ¹H NMR (400 MHz, CDCl₃) δ_{H} 2.19 (m, 4H), 2.14-1.83 (m, 6H), 1.64-1.22 (m, 10H), 1.02 (s, 3H), 0.96 (s, 3H), 0.94-0.64 (m, 6H).

### Synthesis of B-8.5

To a solution of BHT (48.0 g, 218 mmol) in toluene (100 mL) under nitrogen at 0° C. was added AlMe₃ (2 M in toluene, 54.5 mL, 109 mmol) dropwise. The mixture was stirred at 25° C. for 1 hour. To the mixture was added a solution of B-8.4 (12.0 g, 36.5 mmol) in DCM (120 mL) dropwise at −70° cand stirred for 1 hour under N₂. EtMgBr (36.3 mL, 109 mmol, 3M in ethyl ether) was added dropwise at −70° C. and the resulting mixture was stirred at −70° C. for 2 hours. The mixture was poured into saturated citric acid (250 mL, 20%) and extracted with EtOAc (2×200 mL). The combined organic layer was dried over Na₂SO₄, filtered, and concentrated. The residue was purified by flash column chromatography (0˜25% of EtOAc in PE) to give B-8.5 (9.00 g, 63%). ¹H NMR (400 MHz, CDCl₃) δ_{H} 2.24-2.17 (m, 1H), 1.94 (s, 3H), 1.90-1.84 (m, 1H), 1.56-1.18 (m, 16H), 1.15-0.95 (m, 3H), 0.93 (s, 3H), 0.90-0.72 (m, 10H), 0.68-0.59 (m, 1H).

### Synthesis of B-8.6

To a mixture of MePPh₃Br (71.4 g, 200 mmol) in THF (350 mL) was added t-BuOK (22.4 g, 200 mmol) at 25° C. under N₂. The resulting mixture was stirred at 45° C. for 30 mins. B-8.5 (9.00 g, 250 mmol) was added in portions below 55° C. and then the mixture was stirred at 80° C. for 18 hours. The mixture was quenched with 10% NH₄Cl aqueous (300 mL) at 25° C. The mixture was extracted with EtOAc (2×300 mL) and the combined organic phase was separated, dried over Na₂SO₄, filtered, and concentrated. Purification by trituration with MeOH/H₂O (1:1, 1000 mL) at reflux provided B-8.6 (9.40 g). ¹H NMR (400 MHz, CDCl₃) δ_{H} 4.75-4.72 (m, 1H), 4.69-4.65 (m, 1H), 1.83-1.69 (m, 5H), 1.49-1.05 (m, 16H), 1.02-0.77 (m, 12H), 0.75 (s, 3H), 0.68-0.59 (m, 1H), 0.12-0.04 (m, 1H).

### Synthesis of B-8.7

To a solution of B-8.6 (9.00 g, 25.2 mmol) in THF (90 mL) was added BH₃·Me₂S (10 mL, 10 M, 100 mmol) at 0° C. under N₂. The mixture was stirred at 25° C. for 16 hours. To the mixture was added ethanol (14.8 mL, 252 mmol), NaOH (50.4 mL, 5M aq., 252 mmol), and H₂O₂ (37.8 mL, 10 M, 378 mmol) dropwise and the mixture was stirred at 25° C. for 1 hour. The mixture was quenched by Na₂SO₃ (50 g) and concentrated and filtered. The solid was washed with EA (200 mL). The organic layer was dried over Na₂SO₄, filtered, and concentrated. The residue was purified by column chromatography (0-25% EA in PE) to give B-8.7 (11.0 g, crude).

### Synthesis of B-8.8 & B-8.8a

To a solution of B-8.7 (11.0 g, 29.3 mmol) in DCM (120 mL) was added N-methylimidazole (4.80 g, 58.6 mmol), TEA (11.3 mL, 87.9 mmol), and TsCl (8.36 g, 43.9 mmol) and the mixture was stirred at 25° C. for 1 hour. The mixture was poured into NaHCO₃ (200 mL, saturated) and the aqueous phase was extracted with DCM (2×200 mL). The combined organic phase was washed with brine (200 mL), dried over anhydrous Na₂SO₄, filtered, concentrated, and separated by SFC (Column: ChiralPak AD-3 150×4.6 mm I.D., 3 um; Mobile phase: A: CO₂ B: ethanol (0.05% DEA); Isocratic: 40% B; Flow rate: 2.5 mL/min) to give B-8.8 (2.00 g, 13%) and B-8.8a (8.00 g, 51%).

B-8.8: ¹H NMR (400 MHz, CDCl₃) δ_{H} 7.76 (d, J=8.4 Hz, 2H), 7.34 (d, J=8.0 Hz, 2H), 3.69-3.63 (m, 1H), 3.53-3.46 (m, 1H), 2.45 (s, 3H), 2.43-2.36 (m, 1H), 1.64-1.32 (m, 11H), 1.26-1.01 (m, 8H), 0.98-0.77 (m, 15H), 0.71-0.55 (m, 3H), 0.02-0.07 (m, 1H).

B-8.8a: ¹H NMR (400 MHz, CDCl₃) δ_{H} 7.78 (d, J=8.4 Hz, 2H), 7.34 (d, J=8.0 Hz, 2H), 4.16-4.04 (m, 1H), 3.59-3.50 (m, 1H), 2.45 (s, 3H), 2.43-2.36 (m, 1H), 1.65-1.29 (m, 9H), 1.27-0.90 (m, 10H), 0.89-0.73 (m, 11H), 0.72-0.51 (m, 7H), 0.07-0.02 (m, 1H).

### Synthesis of B-8.9

To a solution of B-8.8 (2.00 g, 3.78 mmol) in DMF (20 mL) was added KI (3.00 g, 18.1 mmol) at 25° C. and the mixture was stirred at 60° C. for 5 hours. The mixture was poured into water (20 mL) and stirred for 20 mins. The aqueous phase was extracted with EtOAc (3×20 mL). The combined organic phase was washed with brine (2×20 mL), dried over anhydrous Na₂SO₄, filtered, and concentrated to give B-8.9 (2.00 g). ¹H NMR (400 MHz, CDCl₃) δ_{H} 2.98-2.92 (m, 1H), 2.69-2.61 (m, 1H), 2.46-2.35 (m, 1H), 1.67-1.42 (m, 10H), 1.36-1.18 (m, 11H), 1.10-0.95 (m, 4H), 0.90-0.85 (m, 6H), 0.83 (s, 3H), 0.77-0.56 (m, 3H), 0.10-0.04 (m, 1H).

### Synthesis of B-8.10

To a mixture of Zn powder (377 mg, 5.77 mmol) in dry pyridine (8 mL) was added slowly methyl acrylate (496 mg, 5.77 mmol) and NiCl₂ (159 mg, 1.23 mmol) at 25° C. under N₂. After stirring at 60° C. for 2 hours, the mixture was cooled to 25° C. and a solution of B-8.9 (400 mg, 0.826 mmol) was added to the mixture slowly. The reaction mixture was stirred for 1.5 hours at 25° C. and then the mixture was diluted with NH₄Cl (20 mL). The mixture was filtered through a pad of silica gel. The filtrate was extracted with EtOAc (2×20 mL). The organic phase was dried over Na₂SO₄, filtered, and concentrated. The residue was purified by flash column (0-15% EtOAc in PE) to give B-8.10 (300 mg, 82%). ¹H NMR (400 MHz, CDCl₃) δ_{H} 3.66 (s, 3H), 2.31-2.20 (m, 2H), 2.03-1.91 (m, 1H), 1.70-1.50 (m, 8H), 1.49-0.95 (m, 16H), 0.91-0.57 (m, 17H), 0.06-0.03 (m, 1H).

### Synthesis of B-8

To B-8.10 (300 mg, 0.675 mmol) in THF (3 mL) was added MeMgBr (2.24 mL, 3 M, 6.74 mmol) solution dropwise at 25° C. After stirring at 25° C. for 1 hour under N₂, the reaction mixture was poured into NH₄Cl (10 mL, 10% aq.) and extracted with EtOAc (2×10 mL). The combined organic layer was dried over Na₂SO₄, filtered, and concentrated. Purification by flash column chromatography (10˜20% EtOAc in PE) followed by lyophilization provided B-8 (64.0 mg, 59%). ¹H NMR (400 MHz, CDCl₃) δ_{H} 2.07-1.90 (m, 1H), 1.74-1.57 (m, 6H), 1.52-1.30 (m, 12H), 1.29-1.18 (m, 11H), 1.14-0.83 (m, 17H), 0.79-0.52 (m, 4H), 0.08-0.01 (m, 1H). LC-ELSD/MS purity >99%; MS ESI calcd. for C₃₀H₅₂O₂ [M−2H₂O+H]⁺ 409.4, found 409.5.

### Example 23: Synthesis of (R)-5-((2aS,4S,6aS,6bS,8aS,8bS,9aS,10aS,10bR)-4-ethyl-4-hydroxy-6a,8a-dimethyloctadecahydrocyclopropa[3,4]cyclopenta[1,2-a]phenanthren-8b-yl) hexan-2-one (B-9) & (2aS,4S,6aS,6bS,8aS,8bS,9aS,10aS,10bR)-4-ethyl-6a,8a-dimethyl-8b-((2R,5S)-6,6,6-trifluoro-5-hydroxy-5-methylhexan-2-yl)octadecahydrocyclopropa[3,4]cyclopenta[1,2-a]phenanthren-4-ol (B-10) & (2aS,4S,6aS,6bS,8aS,8bS,9aS,10aS,10bR)-4-ethyl-6a,8a-dimethyl-8b-((2R,5R)-6,6,6-trifluoro-5-hydroxy-5-methylhexan-2-yl)octadecahydrocyclopropa[3,4]cyclopenta[1,2-a]phenanthren-4-ol (B-11)

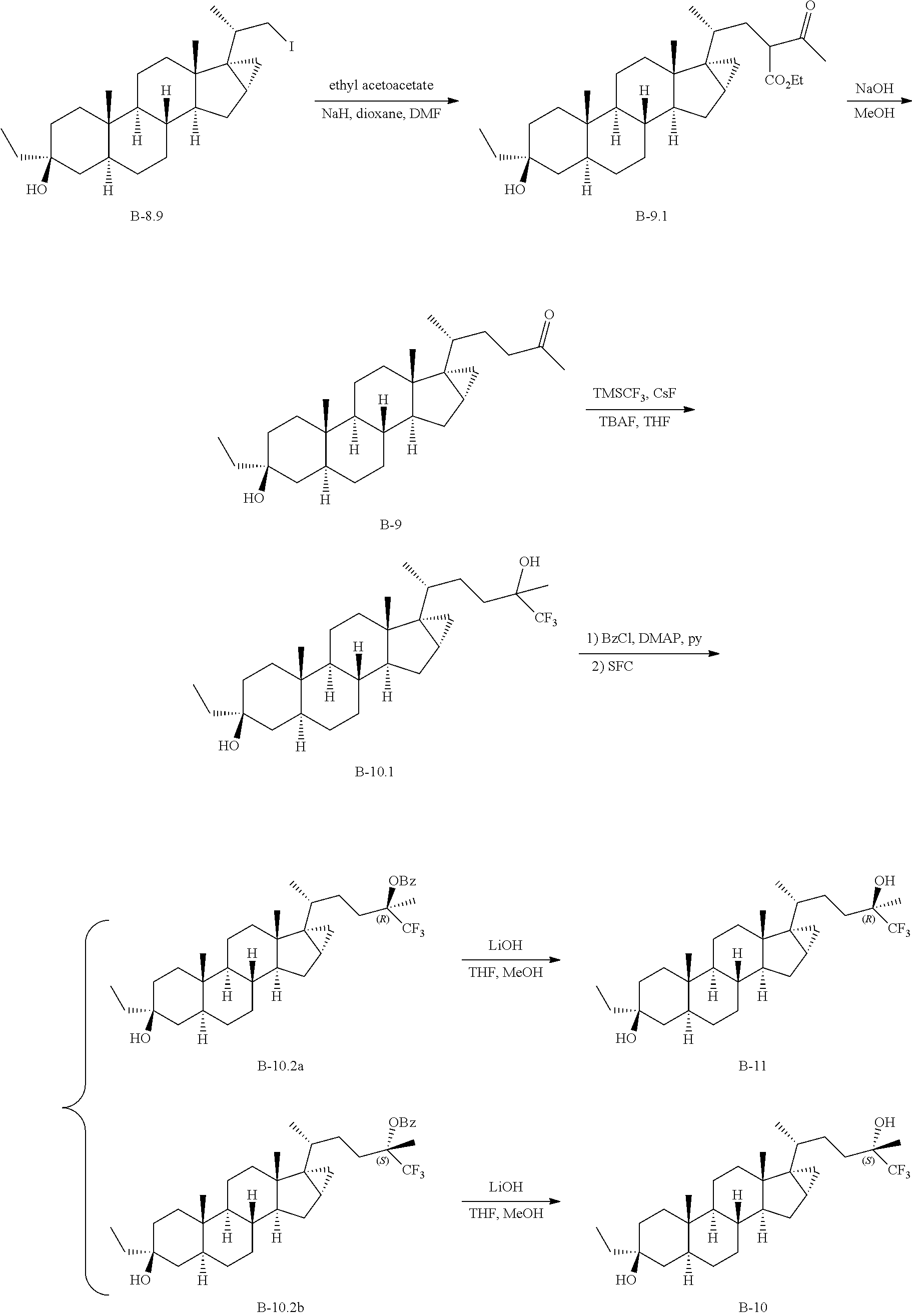

### Synthesis of B-9.1

To a solution of NaH (863 mg, 60%, 21.6 mmol) in dioxane (10 mL) was added ethyl acetoacetate (3.61 g, 27.8 mmol) at 0° C. under N₂ and the mixture was stirred at 0° C. for 1 hour. Then DMF (20 mL) was added. To the mixture was added a solution of B-8.9 (1.50 g, 3.09 mmol) in dioxane (10 mL) dropwise and the reaction mixture was stirred at 105° C. for 16 hours. The mixture was poured into NH₄Cl (20 mL) and the aqueous phase was extracted with DCM (2×20 mL). The combined organic phase was washed with water (2×20 mL), dried over anhydrous Na₂SO₄, filtered, and concentrated. The residue was purified by flash column chromatography (20˜35% of EtOAc in PE) to give B-9.1 (920 mg, 61%). ¹H NMR (400 MHz, CDCl₃) δ_{H} 4.23-4.15 (m, 2H), 3.51-3.39 (m, 1H), 2.24-2.17 (m, 3H), 1.97-1.90 (m, 1H), 1.51-1.33 (m, 9H), 1.30-1.18 (m, 12H), 1.15-0.94 (m, 5H), 0.90-0.84 (m, 8H), 0.82 (s, 3H), 0.80 (s, 3H), 0.66-0.57 (m, 2H), 0.08-0.01 (m, 1H).

### Synthesis of B-9

To a mixture of B-9.1 (920 mg, 1.89 mmol) in MeOH (20 mL) was added H₂O (5 mL) and NaOH (755 mg, 18.9 mmol) at 25° C. The reaction mixture was stirred at 60° C. for 16 hours. The mixture was then concentrated and H₂O (20 mL) was added. The mixture was extracted with EtOAc (3×30 mL). The combined organic phase was washed with saturated brine (15 mL), dried over anhydrous Na₂SO₄, filtered, and concentrated. The residue was purified by silica gel chromatography (20-25% of EtOAc in PE) to give the product B-9 (530 mg, 68%). A sample of B-9 (30 mg) was lyophilized to give B-9 (18.4 mg). ¹H NMR (400 MHz, CDCl₃) δ_{H} 2.46-2.29 (m, 2H), 2.12 (s, 3H), 1.98-1.89 (m, 1H), 1.69-1.57 (m, 5H), 1.54-1.14 (m, 12H), 1.13-0.85 (m, 12H), 0.84-0.82 (m, 6H), 0.79-0.57 (m, 4H), 0.08-0.02 (m, 1H) LC-ELSD/MS purity >99%; MS ESI calcd. for C₂₈H₄₆O₂ [M−H₂O+H]⁺ 397.3, found 397.3.

### Synthesis of B-10.1

To a solution of B-9 (500 mg, 1.20 mmol) in THF (5 mL) was added CsF (729 mg, 4.80 mmol) at 10° C. under N₂. TMSCF₃ (1.02 g, 7.19 mmol) was added dropwise at 10° C. The mixture was stirred at 10° C. for 1 hour. To the mixture was added TBAF (7.19 ml, 1 M in THF, 7.19 mmol). The mixture was stirred at 25° C. for another 16 hours. The mixture was concentrated and the residue was dissolved in EtOAc (10 mL), washed with water (2×10 mL) and brine (10 mL), dried over Na₂SO₄, filtered, and concentrated. The residue was purified by flash column chromatography (0˜30% EtOAc in PE) to give B-10.1 (400 mg, 69%).

### Synthesis of B-10.2a & B-10.2b

To a solution of B-10.1 (400 mg, 0.82 mmol) and DMAP (100 mg, 0.82 mmol) in pyridine (5 mL) was added dropwise benzoyl chloride (1.15 g, 8.25 mmol) at 0° C. The mixture was stirred at 25° C. for 48 hours. The mixture was cooled and then poured into ice-water (10 mL). The aqueous phase was extracted with EtOAc (3×10 mL). The combined organic phase was washed with brine (10 mL), dried over anhydrous Na₂SO₄, filtered, and concentrated. The residue was purified by flash column chromatography (0˜15% of EtOAc in PE) to give B-10.2 (1 g). The residue was dissolved in DCM (20 mL) and NaHCO₃ (20 mL) was added at 20° C. The mixture was stirred at 40° C. for 16 hours. The mixture was cooled and then poured into water (20 mL). The aqueous phase was extracted with DCM (3×20 mL). The combined organic phase was washed with brine (20 mL), dried over anhydrous Na₂SO₄, filtered, and concentrated. Purification by flash column chromatography (3˜5% of EtOAc in PE) followed by SFC (1st run: Column: Phenomenex-Cellulose-2 (250 mm*30 mm, 10 um); Condition: 0.1% NH₃H₂O ETOH; Begin B: 30; End B: 30; Flow Rate (ml/min): 70; Injections: 40; 2^{nd} run: Column: DAICEL CHIRALPAK IG (250 mm*30 mm, 10 um); Condition: 0.1% NH₃H₂O ETOH; Begin B: 40%; End B: 40%; Flow Rate (ml/min): 70; Injections: 30) to give B-10.2a (20 mg, 4%) and B-10.2b (20 mg, 4%).

B-10.2b: ¹H NMR (400 MHz, CDCl₃) δ_{H} 8.01-7.96 (m, 2H), 7.55-7.48 (m, 1H), 7.44-7.37 (m, 2H), 2.20-1.87 (m, 7H), 1.77-1.59 (m, 6H), 1.53-1.42 (m, 3H), 1.30 (s, 3H), 1.28-1.06 (m, 9H), 0.91 (s, 3H), 0.91-0.86 (m, 7H), 0.85 (s, 3H), 0.83-0.58 (m, 4H), 0.13-0.04 (m, 1H) ¹⁹F NMR (376 MHz, CDCl₃) δ_{F} −83.20 (s)

B-10.2a: ¹H NMR (400 MHz, CDCl₃) δ_{H} 8.03-7.93 (m, 2H), 7.55-7.48 (m, 1H), 7.43-7.37 (m, 2H), 2.18-1.85 (m, 8H), 1.78 (s, 1H), 1.70-1.59 (m, 4H), 1.54-1.42 (m, 4H), 1.32 (s, 3H), 1.30-1.00 (m, 10H), 0.91 (s, 3H), 0.91-0.86 (m, 6H), 0.85 (s, 3H), 0.80-0.58 (m, 3H), 0.14-0.00 (m, 1H) ¹⁹F NMR (376 MHz, CDCl₃) δ_{F} −82.88 (s).

### Synthesis of B-10

To a solution of B-10.2b (20 mg, 0.033 mmol) in THF (0.5 mL) was added MeOH (1 mL), H₂O (0.2 mL), and LiOH·H₂O (99.4 mg) at 25° C. The mixture was stirred at 50° C. for 120 hours. The mixture was diluted with water (10 mL) and extracted with EtOAc (2×10 mL). The combined organic layer was dried over Na₂SO₄, filtered, and concentrated. Purification by flash column chromatography (0˜25% EtOAc in PE) followed by lyophilization provided B-10 (4.20 mg, 28%). ¹H NMR (400 MHz, CDCl₃) δ_{H} 1.99-1.88 (m, 1H), 1.77 (s, 1H), 1.71-1.58 (m, 6H), 1.54-1.33 (m, 7H), 1.30 (s, 3H), 1.27-0.92 (m, 11H), 0.91-0.72 (m, 15H), 0.66-0.56 (m, 2H), 0.10-0.01 (m, 1H) ¹⁹F NMR (376 MHz, CDCl₃) &F-83.20 (s) LC-ELSD/MS purity >99%; MS ESI calcd. for C₂₉H₄₇F₃O₂ [M−H₂O+H]⁺ 467.4, found 467.4.

### Synthesis of B-11

To a solution of B-10.2a (20 mg, 0.033 mmol) in THF (0.5 mL) was added MeOH (1 mL), H₂O (0.2 mL) and LiOH·H₂O (99.4 mg) at 25° C. The mixture was stirred at 50° C. for 120 hours. The mixture was diluted with water (10 mL) and extracted with EtOAc (2×10 mL). The combined organic layer was dried over Na₂SO₄, filtered, and concentrated. Purification by flash column chromatography (0˜25% EtOAc in PE) followed by lyophilization provided B-11 (6.20 mg, 42%). ¹H NMR (400 MHz, CDCl₃) δ_{H} 1.97-1.87 (m, 1H), 1.77 (s, 1H), 1.73-1.57 (m, 7H), 1.54-1.34 (m, 7H), 1.32 (s, 3H), 1.26-0.95 (m, 10H), 0.93-0.70 (m, 15H), 0.67-0.55 (m, 2H), 0.10-0.02 (m, 1H) ¹⁹F NMR (376 MHz, CDCl₃) δ_{F} −82.89 (s) LC-ELSD/MS purity >99%; MS ESI calcd. for C₂₉H₄₇F₃O₂ [M−H₂O+H]⁺ 467.4, found 467.3.

### Example 24: Synthesis of (3S,5S,8R,9S,10S,13R,14S,15S,17R)-3-ethyl-17-((R)-6-hydroxy-6-methylheptan-2-yl)-10,13,15-trimethylhexadecahydro-1H-cyclopenta[a]phenanthren-3-ol (B-12)

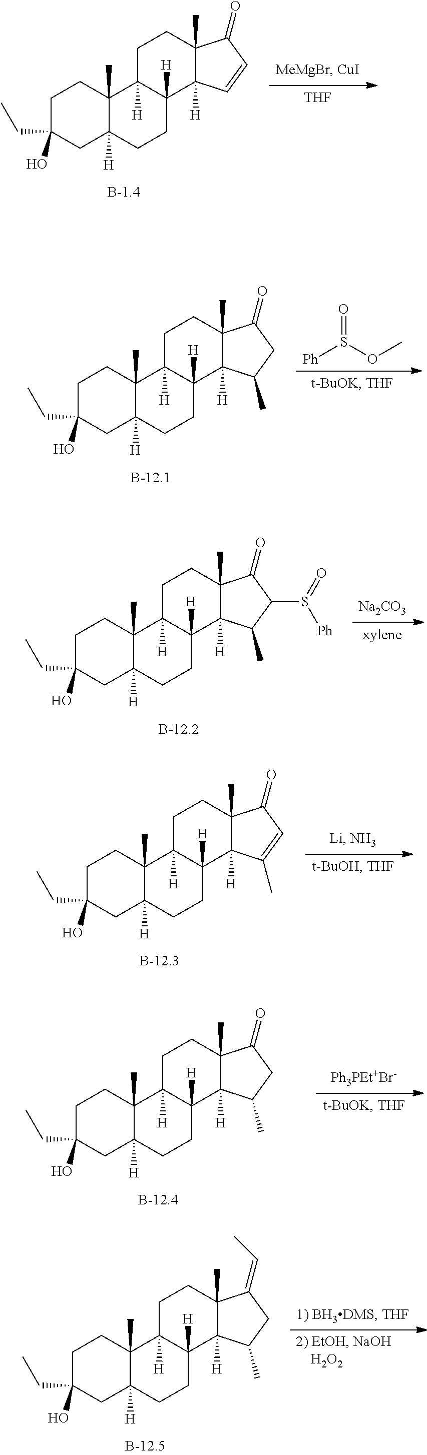

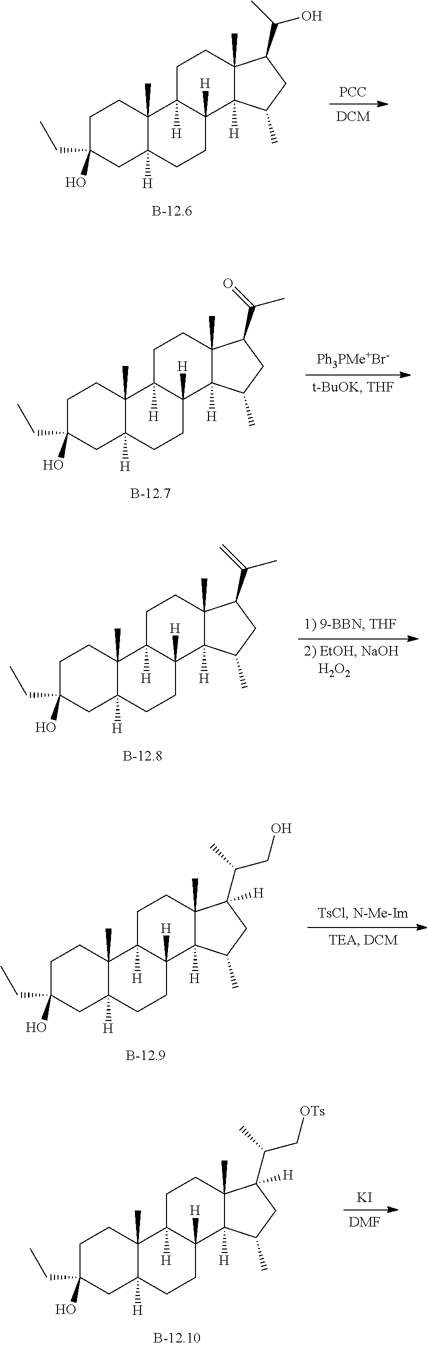

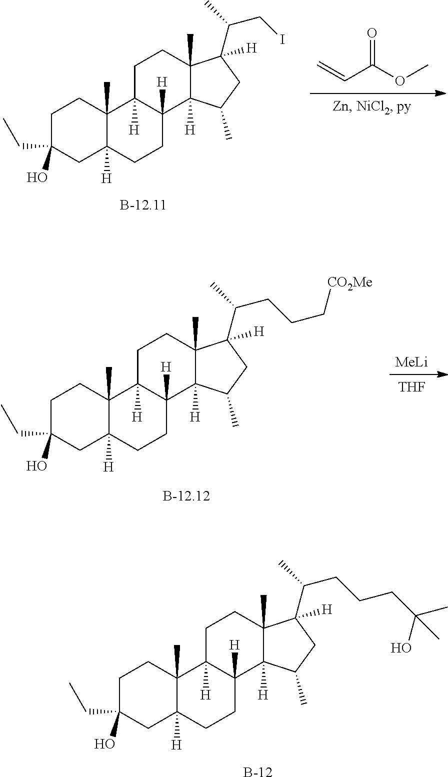

### Synthesis of B-12.1

To a solution of MeMgBr (42.0 mL, 126 mmol, 3M) in THF (200 mL) was added CuI (17.9 g, 94.5 mmol) at 0° C. and the mixture was stirred at 0° C. for 1 hour. The mixture was reacted with B-1.4 (10.0 g, 31.5 mmol) in THF (100 mL) at 0° C. and the mixture was stirred for 3 hours. The mixture was poured into saturated NH₄Cl (200 mL) and extracted with EtOAc (3×100 mL). The combined organic layer was washed with brine (200 mL), dried over anhydrous Na₂SO₄, filtered, and concentrated. The residue was purified by flash column chromatography (10%˜25% of EtOAc in PE) to give B-12.1 (7.80 g, 75%). ¹H NMR (400 MHz, CDCl₃) δ_{H} 2.50-2.36 (m, 2H), 2.28-2.18 (m, 1H), 1.93-1.63 (m, 5H), 1.43-1.13 (m, 11H), 1.13-1.09 (m, 3H), 1.06-0.70 (m, 14H).

### Synthesis of B-12.2

To a solution of t-BuOK (5.25 g, 46.8 mmol) in THF (100 mL) was added B-12.1 (7.80 g, 23.4 mmol) at 25° C. under N₂ and the mixture was stirred for 10 mins. Methyl benzenesulfinate (7.31 g, 46.8 mmol) was added and the mixture was stirred at 25° C. for 1 hour. The mixture was quenched with H₂O (200 mL) and extracted with EtOAc (3×50 mL). The organic layer was separated, washed with brine (2×200 mL) dried over Na₂SO₄, filtered, and concentrated to give B-12.2 (11.0 g).

### Synthesis of B-12.3

To a mixture of B-12.2 (11.0 g, 24.0 mmol) in xylene (100 mL) was added Na₂CO₃ (38.1 g, 360 mmol) in portions. The reaction mixture was stirred at 140° C. for 12 hours under N₂. The mixture was filtered and concentrated. The residue was purified by silica gel chromatography (0˜30% of EtOAc in PE) to give the product B-12.3 (510 mg, 5%). 1H NMR (400 MHz, CDCl₃) δ_{H} 5.70 (d, J 1.2 Hz, 1H), 2.26-2.19 (m, 2H), 2.15 (s, 3H), 2.04-1.65 (m, 4H), 1.58-1.13 (m, 13H), 1.09-0.96 (m, 4H), 0.90 (s, 3H), 0.90-0.78 (m, 4H) LC-ELSD/MS purity >99%, MS ESI calcd. for C₂₂H₃₄O₂ [M+H]⁺ 331.3, found 331.2.

### Synthesis of B-12.4

Lithium (839 mg, 121 mmol) was added to freshly prepared liquid ammonia (100 mL) in portions at -70° C. After stirring at −70° C. for 1 hour, a solution of B-8.3 (8.00 g, 24.2 mmol) in dry THF (100 mL) and t-butanol (7.16 g, 96.8 mmol) were added to this mixture under vigorous stirring and the temperature was maintained below −60° C. The resulting mixture was stirred at −70° C. for 1 hour. Ammonium chloride (30.0 g) was added to reaction mixture and the mixture was warmed to 20° C. and stirred for 16 hours. The reaction mixture was added to H₂O (200 mL) and extracted with EtOAc (3×100 mL). The organics were washed with brine (200 mL), dried over Na₂SO₄, filtered, and concentrated. Purification by flash column chromatography (0˜30% of EtOAc in PE) provided B-12.4 (4.20 g, 55%). 1H NMR (400 MHz, CDCl₃) δ_{H} 2.73 (dd, J 8.4, 19.2 Hz, 1H), 2.23-2.08 (m, 1H), 2.03-1.93 (m, 1H), 1.80-1.59 (m, 7H), 1.56-1.21 (m, 9H), 1.15 (d, J 6.4 Hz, 3H), 1.14-0.94 (m, 4H), 0.92-0.84 (m, 9H), 0.79-0.69 (m, 1H) LC-ELSD/MS purity >99%, MS ESI calcd. for C22H₃₆₀₂ [M+H]⁺ 333.3, found 333.3.

### Synthesis of B-12.5

To a solution of EtPPh₃Br (13.3 g, 36.0 mmol) in THF (40 mL) was added t-BuOK (4.03 g, 36.0 mmol) and the mixture was stirred at 45° C. for 10 mins. B-12.4 (6.00 g, 18.0 mmol) in THF (10 mL) was added and the mixture was stirred at 45° C. for 16 hours. The mixture was poured into saturated NH₄Cl (100 mL) and extracted with EtOAc (3×50 mL). The combined organic layer was washed with brine (100 mL), dried over anhydrous Na₂SO₄, filtered, and concentrated. The residue was purified by flash column chromatography (0˜30% of EtOAc in PE) to give B-12.5 (4.80 g). ¹H NMR (400 MHz, CDCl₃) δ_{H} 5.12-5.00 (m, 1H), 2.72-2.59 (m, 1H), 2.24-2.16 (m, 1H), 1.96-1.61 (m, 8H), 1.52-1.06 (m, 14H), 1.02 (d, J 6.0 Hz, 3H), 0.92 (s, 3H), 0.90-0.86 (m, 4H), 0.84 (s, 3H), 0.83-0.63 (m, 2H).

### Synthesis of B-12.6

To a solution of B-12.5 (4.80 g, 13.9 mmol) in THF (50 mL) was added BH₃·Me₂S (6.95 mL, 69.5 mmol, 10 M) at 0° C. under N₂. The mixture was stirred at 25° C. for 16 hours. To the resulting mixture was added ethanol (24.2 mL, 417 mmol) at 25° C., followed by NaOH (41.6 mL, 5.0 M, 208 mmol) at 0° C. Hydrogen peroxide (20.8 mL, 10 M, 208 mmol) was added dropwise at 0° C. The mixture was stirred at 80° C. for 1 hour. The mixture was cooled to 25° C. and Na₂S₂O₃ (100 mL, sat.) was added. The aqueous was extracted with EtOAc (3×100 mL) and the combined organic layer was washed with brine (2×50 mL), dried over Na₂SO₄, filtered, and concentrated to give B-12.6 (5.30 g). ¹H NMR (400 MHz, CDCl₃) δ_{H} 3.79-3.63 (m, 1H), 2.01-1.70 (m, 4H), 1.50-1.30 (m, 7H), 1.25-1.18 (m, 7H), 1.17-0.92 (m, 10H), 0.91-0.67 (m, 13H).

### Synthesis of B-12.7

To a solution of B-12.6 (4.80 g, 13.2 mmol) in DCM (90 mL) was added silica gel (8.49 g) and PCC (8.49 g, 39.5 mmol) at 25° C. The mixture was stirred at 25° C. for 3 hours. The suspension was filtered and the filter cake was washed with DCM (2×20 mL). The combined filtrate was concentrated, then purified by flash column chromatography (0˜30% of EtO Ac in PE) to give B-12.7 (3.30 g, 69%). ¹H NMR (400 MHz, CDCl₃) δ_{H} 2.59-2.43 (m, 2H), 2.10 (s, 3H), 1.96-1.89 (m, 1H), 1.86-1.64 (m, 4H), 1.51-1.28 (m, 7H), 1.23-1.07 (m, 5H), 1.05 (d, J 6.4 Hz, 3H), 1.03-0.92 (m, 3H), 0.88 (t, J 7.6 Hz, 3H), 0.85-0.69 (m, 6H), 0.66 (s, 3H).

### Synthesis of B-12.8

To a mixture of MePPh₃Br (5.93 g, 16.6 mmol) in THF (25 mL) was added t-BuOK (1.86 g, 16.6 mmol) at 25° C. under N₂. The resulting mixture was stirred at 55° C. for 30 mins. B-12.7 (2.00 g, 5.54 mmol) was added in portions below 50° C. The mixture was stirred at 55° C. for 3 hours. The reaction mixture was quenched with 10% NH₄Cl aqueous (50 mL), extracted with EtOAc (2×50 mL), washed with brine (40 mL), dried over Na₂SO₄, filtered, and concentrated. The residue was purified by flash column chromatography (0˜20% of EtOAc in PE) to give B-12.8 (3.10 g, 95%). ¹H NMR (400 MHz, CDCl₃) δ_{H} 4.84 (s, 1H), 4.66 (s, 1H), 2.22-2.05 (m, 2H), 1.84-1.70 (m, 6H), 1.54-1.29 (m, 7H), 1.22-1.06 (m, 6H), 1.05 (d, J 6.8 Hz, 3H), 1.02-0.91 (m, 3H), 0.90-0.83 (m, 7H), 0.82-0.67 (m, 3H), 0.60 (s, 3H).

### Synthesis of B-12.9

A solution of B-12.8 (2.70 g, 7.52 mmol) in THF (30 mL) was added 9-BBN dimer (5.49 mg, 22.5 mmol) at 0° C. under N₂ then the reaction was stirred at 25° C. for 16 hours. To the resulting mixture was added ethanol (7.87 mL, 135 mmol) at 25° C., followed by NaOH (27.0 mL, 5.0 M, 135 mmol) at 0° C. Hydrogen peroxide (13.5 mL, 10 M, 135 mmol) was added dropwise at 0° C. and the reaction mixture was stirred at 80° C. for 1 hour. The mixture was cooled to 25° C. and Na₂S₂O₃ (100 mL, sat. aq.) was added. The aqueous was extracted with EtOAc (3×50 mL). The combined organic layer was washed with brine (2×50 mL), dried over Na₂SO₄, filtered, and concentrated to give B-12.9 (7.00 g). ¹H NMR (400 MHz, CDCl₃) δ_{H} 3.58 (dd, J 3.2, 10.4 Hz, 1H), 3.32 (dd, J 6.8, 10.4 Hz, 1H), 1.79-1.68 (m, 9H), 1.50-1.34 (m, 14H), 1.17-0.98 (m, 9H), 0.88-0.80 (m, 6H), 0.73-0.60 (m, 4H).

### Synthesis of B-12.10

To a solution of B-12.9 (7.00 g, 18.5 mmol) in DCM (40 mL) was added TEA (15.2 mL, 110 mmol), N-Me-Im (4.40 mL, 55.4 mmol) and TsCl (10.5 g, 55.4 mmol) and the mixture was stirred at 20° C. for 4 hours. To the mixture was added water (200 mL) and the mixture was adjusted to pH-3 with HCl (30 mL), The aqueous was extracted with DCM (3×50 mL), the combined organic layer was washed NaHCO₃ (100 mL), brine (2×100 mL), dried over Na₂SO₄, filtered, and concentrated. Purification by flash column (0˜30% of EtOAc in PE) and further purification by SFC twice (1st run: Column: DAICEL CHIRALCEL OJ (250 mm*50 mm, 10 um); Condition: 0.1% NH₃H₂O ETOH; Begin B: 20%; End: 20%. 2nd run: Column: Phenomenex-Cellulose-2 (250 mm*50 mm, 10 um); Condition: 0.1% NH₃H₂O ETOH; Begin B: 45%; End B: 45%) provided B-12.10 (1.70 g, 78%). 1H NMR (400 MHz, CDCl₃) δ_{H} 7.78 (d, J 8.4 Hz, 2H), 7.34 (d, J 8.0 Hz, 2H), 3.95 (dd, J 3.2, 9.2 Hz, 1H), 3.74 (dd, J 6.4, 9.2 Hz, 1H), 2.44 (s, 3H), 1.88-1.57 (m, 8H), 1.53-0.99 (m, 15H), 0.98-0.92 (m, 7H), 0.87 (t, J 7.2 Hz, 3H), 0.81 (s, 3H), 0.70-0.61 (m, 5H).

### Synthesis of B-12.11

To a solution of B-12.10 (1.70 g, 3.20 mmol) in DMF (20 mL) was added KI (2.32 g, 14.0 mmol) at 25° C. and the mixture was stirred at 60° C. for 1 hour. The mixture was poured into water (100 mL), stirred for 20 mins, filtered, and concentrated to give B-12.11 (1.67 g). ¹H NMR (400 MHz, CDCl₃) δ_{H} 3.35-3.28 (m, 1H), 3.12 (dd, J 5.6, 9.6 Hz, 1H), 1.90-1.64 (m, 5H), 1.51-1.10 (m, 16H), 1.05-0.85 (m, 12H), 0.83 (s, 3H), 0.76-0.66 (m, 5H).

### Synthesis of B-12.12

To a mixture of Zn powder (187 mg, 2.87 mmol) in dry pyridine (2 mL) was added slowly methyl prop-2-enoate (247 mg, 2.87 mmol) and NiCl₂·6H₂O (102 mg, 0.62 mmol) at 20° C. under N₂. After stirring at 60° C. for 2 hours, the mixture was cooled to 20° C. and a solution of B-12.11 (200 mg, 0.41 mmol) in pyridine (1 mL) was added slowly. The reaction mixture was stirred for 1.5 hours at 20° C. then diluted with NH₄Cl (50 mL). The mixture was filtered through a pad of silica gel. The filtrate was extracted with EtOAc (2×30 mL) and the organic phase was dried over Na₂SO₄, filtered and concentrated. The residue was purified by flash column chromatography (0-20% of EtOAc in PE) to give B-12.12 (130 mg, 71%). 1H NMR (400 MHz, CDCl₃) δ_{H} 3.67 (s, 3H), 2.34-2.18 (m, 2H), 1.96-1.86 (m, 1H), 1.84-1.75 (m, 1H), 1.73-1.59 (m, 5H), 1.54-1.07 (m, 18H), 1.05-0.93 (m, 6H), 0.93-0.85 (m, 6H), 0.83 (s, 3H), 0.75-0.63 (m, 5H).

### Synthesis of B-12

To a solution of B-12.12 (100 mg, 0.22 mmol) in THF (2 mL) was added methyllithium (1 mL, 1.6 mmol, 1.6 M in Et₂O) dropwise at 0° C. under N₂. The reaction mixture was stirred at 25° C. for 2 hours. The reaction mixture was quenched by saturated NH₄Cl (30 mL) and extracted with ethyl acetate (3×30 mL). The combined organic phase was washed with brine (2×100 mL), dried over anhydrous Na₂SO₄, filtered, and concentrated. The residue was purified by flash column chromatography (0˜25% of EtOAc in PE) to give B-12 (65.0 mg, 65%). ¹H NMR (400 MHz, CDCl₃) δ_{H} 1.96-1.87 (m, 1H), 1.84-1.76 (m, 1H), 1.72-1.57 (m, 6H), 1.56-1.23 (m, 15H), 1.21 (s, 6H), 1.19-1.03 (m, 6H), 1.02-0.85 (m, 11H), 0.83 (s, 3H), 0.72-0.62 (m, 5H) LC-ELSD/MS purity >99%, MS ESI calcd. for C₃₀H₅₄O₂ [M−2H₂O+H]⁺ 411.4, found 411.4.

### Example 25: Synthesis of (3S,5S,8R,9S,10S,13R,14S,15S,17R)-3-ethyl-10,13,15-trimethyl-17-((2R,5R)-6,6,6-trifluoro-5-hydroxy-5-methylhexan-2-yl)hexadecahydro-1H-cyclopenta[a]phenanthren-3-ol (B-13) & (3S,5S,8R,9S,10S,13R,14S,15S,17R)-3-ethyl-10,13,15-trimethyl-17-((2R,5S)-6,6,6-trifluoro-5-hydroxy-5-methylhexan-2-yl)hexadecahydro-1H-cyclopenta[a]phenanthren-3-ol (B-14)

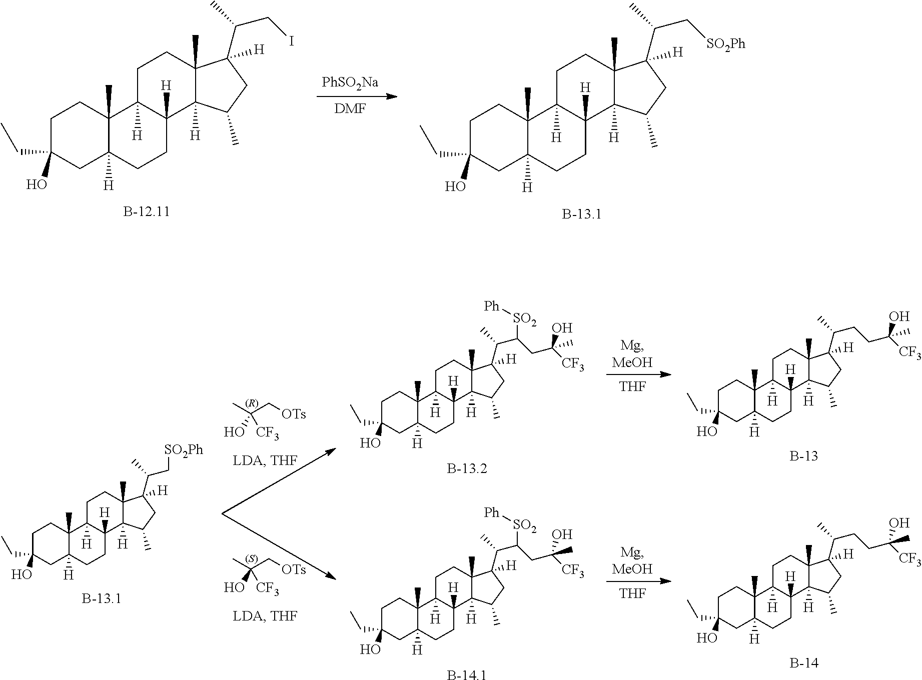

### Synthesis of B-13.1

To a solution of B-12.11 (0.970 g, 1.99 mmol) in DMF (10 mL) was added sodium benzenesulfinate (326 mg, 1.99 mmol) and the mixture was stirred at 60° C. for 2 hours. The mixture was poured into water (200 mL) and filtered. The filter cake was washed with water (2×50 mL). The resulting filter cake was dissolved in DCM (200 mL), washed with brine (200 mL), dried over Na₂SO₄, filtered, and concentrated. The residue was purified by flash column chromatography (0˜30% EtOAc in PE) to give B-13.1 (700 mg, 70%). ¹H NMR (400 MHz, CDCl₃) δ_{H} 7.93-7.88 (m, 2H), 7.67-7.61 (m, 1H), 7.60-7.53 (m, 2H), 3.12 (dd, J=1.2, 14.4 Hz, 1H), 2.83 (dd, J=10.0, 14.0 Hz, 1H), 2.13-2.05 (m, 1H), 1.91-1.61 (m, 3H), 1.59-1.27 (m, 11H), 1.22-0.90 (m, 15H), 0.86 (t, J=7.2 Hz, 3H), 0.81 (s, 3H), 0.72-0.60 (m, 5H).

### Synthesis of B-13.2

To a solution of B-13.1 (270 mg, 0.54 mmol) in THF (4 mL) was added n-BuLi (1.29 mL, 3.23 mmol, 2.5M in hexane) dropwise at -70° C. under N₂. The reaction mixture was stirred at -70° C. for 0.5 hour. DIPA (0.454 ml, 3.23 mmol) was added dropwise at −70° C. for 0.5 hour. A solution of (2R)-1,1,1-trifluoro-2-methyl-3-[(4-methylbenzenesulfonyl) oxy]propan-2-ol (241 mg, 0.81 mmol) in THF (1 ml) was added dropwise at −70° C. The reaction mixture was warmed to 25° C. slowly, and stirred at 25° C. for 18 hours. The reaction mixture was quenched with saturated NH₄Cl aqueous (50 mL) and extracted with EtOAc (2×20 mL). The combined organic phase was washed with brine (50 mL), dried over Na₂SO₄, filtered, concentrated to give B-13.2 (260 mg, crude) as an off-white solid.

### Synthesis of B-13

To a solution of B-13.2 (260 mg, 0.41 mmol) in methanol (10 mL) and THF (5 mL) was added nickel (II) chloride (10.6 mg, 0.08 mmol) and Mg powder (396 mg, 16.5 mmol) in four portions under N₂ at 65° C. The reaction mixture was stirred at 65° C. for 1 hour. The mixture was quenched by citric acid (100 mL, 10%) which was added dropwise until solid was dissolved. The mixture was extracted with EtOAc (3×50 mL), the organic layer was washed with sat. NaHCO₃ (100 mL), brine (100 mL), dried over Na₂SO₄, filtered, and concentrated. The residue was purified by flash column (0˜20% of EtOAc in PE) to give B-13 (50.0 mg). To a solution of B-13 (50.0 mg) in THF (10 mL) was added Pd—C(wet, 10%, 50.0 mg) under N₂. The suspension was degassed under vacuum and purged with H₂ three times. The mixture was stirred under H₂ (30 psi) at 30° C. for 48 hours. The mixture was filtered through a pad of Celite, washed with THF (3×50 mL), and the filtrate was concentrated. The residue was purified by column chromatography (0˜20% of EtOAc in PE) to give B-13 (40 mg, 15%). ¹H NMR (400 MHz, CDCl₃) δ_{H} 1.94-1.61 (m, 7H), 1.57-1.32 (m, 14H), 1.30-0.96 (m, 14H), 0.92 (d, J 6.4 Hz, 3H), 0.89-0.85 (m, 3H), 0.83 (s, 3H), 0.75-0.61 (m, 5H) ¹⁹F NMR (376 MHz, CDCl₃) δ_{F} −82.77 (s) LC-ELSD/MS purity >99%, MS ESI calcd. for C₂₉H₄₉F₃O₂ [M−H₂O+H]⁺ 469.3, found 469.4.

### Synthesis of B-14.1

To a solution of B-13.1 (500 mg, 1.00 mmol) in THF (4 mL) was added n-BuLi (1.99 mL, 4.99 mmol, 2.5M in hexane) dropwise at -70° C. under N₂. The reaction mixture was stirred at −70° C. for 0.5 hour. DIPA (0.7 mL, 4.99 mmol) was added dropwise at −70° C. for 0.5 hour. A solution of (2S)-1,1,1-trifluoro-2-methyl-3-[(4-methylbenzenesulfonyl)oxy]propan-2-ol (444 mg, 1.49 mmol) in THF (1 mL) was added dropwise at −70° C. The reaction mixture was warmed to 25° C. slowly and stirred for 18 hours. The reaction mixture was quenched with saturated NH₄Cl (50 mL) and extracted with EtOAc (2×20 mL). The combined organic phase was washed with brine (50 mL), dried over Na₂SO₄, filtered, and concentrated to give B-14.1 (600 mg).

### Synthesis of B-14

To a solution of B-14.1 (600 mg, 0.96 mmol) in methanol (40 mL) and THF (10 mL) was added nickel (II) chloride (24.6 mg, 0.19 mmol) and Mg powder (916 mg, 38.2 mmol) in four portions under N₂ at 65° C. The reaction mixture was stirred at 65° C. for 1 hour. The reaction mixture was quenched with citric acid (100 mL, 10%) which was added dropwise until solid was dissolved. The mixture was extracted with EtOAc (3×50 mL), the organic layer was washed with sat. NaHCO₃ (100 mL), brine (100 mL), dried over Na₂SO₄, filtered, and concentrated. The residue was purified by flash column chromatography (0˜20% of EtOAc in PE) to give B-14 (145.1 mg, 31%). ¹H NMR (400 MHz, CDCl₃) δ_{H} 1.95-1.86 (m, 1H), 1.84-1.59 (m, 7H), 1.57-1.33 (m, 11H), 1.32 (s, 3H), 1.28-0.95 (m, 13H), 0.95-0.85 (m, 6H), 0.83 (s, 3H), 0.75-0.63 (m, 5H) ¹⁹F NMR (376 MHz, CDCl₃) δ_{F} −83.13 (s) LC-ELSD/MS purity >99%, MS ESI calcd. for C₂₉H₄₉F₃O₂ [M−H₂O+H]⁺ 469.4, found 469.4.

### Example 26: Synthesis of (3S,5S,8R,9S,10S,13R,14S,15R,17R)-3-ethyl-10,13,15-trimethyl-17-((2R,5S)-6,6,6-trifluoro-5-hydroxy-5-methylhexan-2-yl)hexadecahydro-1H-cyclopenta[a]phenanthren-3-ol (B-15) & (3S,5S,8R,9S,10S,13R,14S,15R,17R)-3-ethyl-10,13,15-trimethyl-17-((2R,5R)-6,6,6-trifluoro-5-hydroxy-5-methylhexan-2-yl)hexadecahydro-1H-cyclopenta[a]phenanthren-3-ol (B-16)

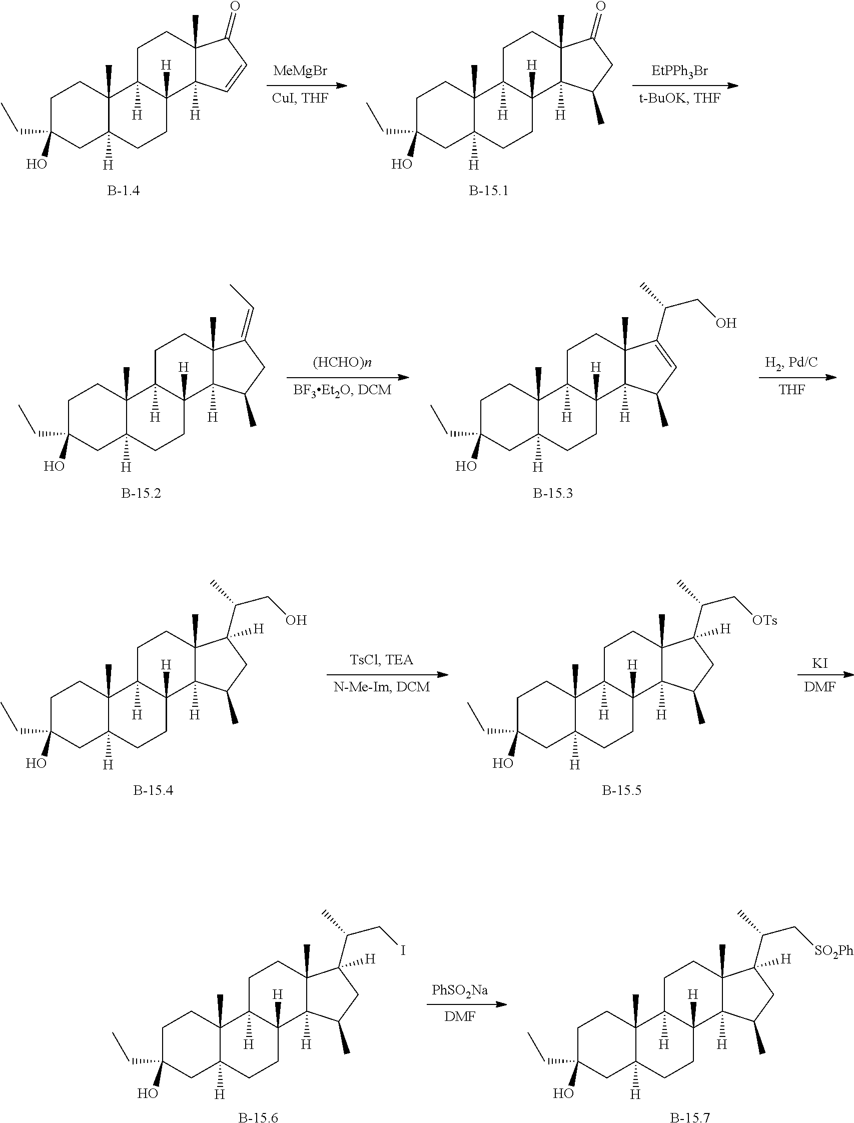

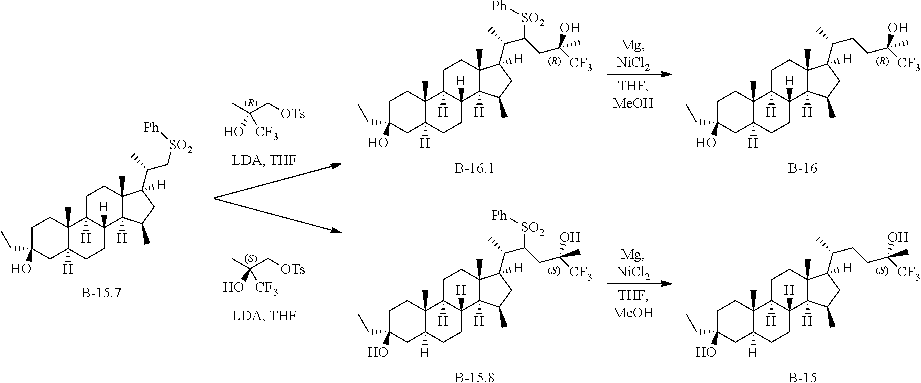

### Synthesis of B-15.1

To a solution of MeMgBr (420 mL, 126 mmol, 3M) in THF (200 mL) was added CuI (17.9 g, 94.5 mmol) at 0° C. and the mixture was stirred for 1 hour. The mixture was reacted with B-1.4 (10.0 g, 31.5 mmol) in THF (50 mL) was added at 0° C. and stirred for 3 hours. The mixture was poured into saturated NH₄Cl (200 mL) and extracted with EtOAc (3×100 mL). The combined organic layer was washed with brine (200 mL), dried over anhydrous Na₂SO₄, filtered, and concentrated. Purification by flash column chromatography (10%˜25% of EtOAc in PE) provided B-15.1 (8.70 g, 84%). ¹H NMR (400 MHz, CDCl₃) δ_{H} 2.49-2.37 (m, 2H), 2.23 (d, J=17.2 Hz, 1H), 1.93-1.86 (m, 1H), 1.80-1.62 (m, 4H), 1.54-1.30 (m, 8H), 1.24-1.13 (m, 4H), 1.11 (d, J=7.2 Hz, 3H), 1.01 (s, 3H), 0.98-0.77 (m, 10H).

### Synthesis of B-15.2

To a mixture of EtPPh₃Br (29.0 g, 78.3 mmol) in THF (120 mL) was added t-BuOK (8.78 g, 78.3 mmol) at 20° C. under N₂. The resulting mixture was stirred at 40° C. for 30 mins. B-15.1 (8.70 g, 26.1 mmol) in THF (20 mL) was added in portions below 40° C. and the mixture was stirred at 40° C. for 16 hours. The reaction mixture was quenched with 10% NH₄Cl aqueous (150 mL) at 20° C. and the THF layer was separated. The aqueous was extracted with EtOAc (3×100 mL). The combined organic phase was washed with brine (2×200 mL), filtered, and concentrated. The residue was triturated with MeOH/H₂O (200 mL/200 mL) and the filtered solid was purified by flash column chromatography (0˜30% of EtOAc in PE) to give B-15.2 (7.00 g, 95%). ¹H NMR (400 MHz, CDCl₃) δ_{H} 5.19-5.05 (m, 1H), 2.63-2.47 (m, 1H), 2.20-2.06 (m, 3H), 1.88-1.80 (m, 1H), 1.68-1.58 (m, 8H), 1.53-1.27 (m, 8H), 1.18-1.11 (m, 1H), 1.08 (s, 3H), 1.03-0.86 (m, 13H), 0.77-0.86 (m, 1H).

### Synthesis of B-15.3

To a solution of B-15.2 (3.50 g, 10.1 mmol) in DCM (80 mL) was added (HCHO) n (4.53 g, 50.4 mmol) and BF₃·Et₂O (0.25 mL, 2.02 mmol) at 25° C. and the mixture was stirred for 2 hours. The mixture was filtered and the filter cake was washed with DCM (2×50 mL). The filtrate was concentrated and purified by flash column (0˜30% of EtOAc in PE) to give B-15.3 (490 mg). ¹H NMR (400 MHz, CDCl₃) δ_{H} 5.48 (d, J=2.4 Hz, 1H), 3.63-3.51 (m, 2H), 2.59-2.31 (m, 3H), 1.94-1.76 (m, 2H), 1.72-1.65 (m, 3H), 1.46-1.36 (m, 5H), 1.19-1.05 (m, 4H), 1.04-0.95 (m, 12H), 0.90-0.78 (m, 10H).

### Synthesis of B-15.4

To a mixture of B-15.3 (490 mg, 1.30 mmol) in THF (5 mL) was added Pd/C (dry, 10%, 100 mg) under N₂. The suspension was degassed under vacuum and purged with H₂ three times. The mixture was stirred under H₂ (50 psi) at 50° C. for 24 hours. The mixture was filtered, washed with THF (2×30 mL), concentrated, and purified by flash column chromatography (0˜30% of EtOAc in PE) to provide B-15.4 (370 mg, 76%). ¹H NMR (400 MHz, CDCl₃) δ_{H} 3.70-3.55 (m, 1H), 3.45-3.40 (m, 1H), 2.28-2.17 (m, 2H), 1.93-1.86 (m, 2H), 1.70-1.61 (m, 3H), 1.49-1.35 (m, 8H), 1.22-1.05 (m, 7H), 1.01-0.92 (m, 5H), 0.90-0.83 (m, 15H).

### Synthesis of B-15.5

To a solution of B-15.4 (370 mg, 0.98 mmol) in DCM (5 mL) was added TEA (0.54 mL, 3.92 mmol), N-Me-Im (0.16 mL, 1.96 mmol) and TsCl (373 mg, 1.96 mmol) and the mixture was stirred at 20° C. for 2 hours. Water was added (50 mL) and the mixture was adjusted to pH=3 with HCl (2M, 10 mL). The aqueous layer was extracted with DCM (3×30 mL) and the combined organic layer was washed with NaHCO₃ (50 mL) and brine (2×100 mL), dried over Na₂SO₄, filtered, and concentrated. The residue was purified by flash column chromatography (0˜30% of EtOAc in PE) and further purified by SFC (Column: DAICEL CHIRALPAK AD (250 mm*30 mm, 10 um); Condition: 0.1% NH₃H₂O ETOH; Begin B: 45%; End B: 45%) to afford B-15.5 (140 mg, 47%). ¹H NMR (400 MHz, CDCl₃) δ_{H} 7.78 (d, J=8.4 Hz, 2H), 7.34 (d, J=8.0 Hz, 2H), 3.95 (dd, J=3.2, 9.2 Hz, 1H), 3.80 (dd, J=6.4, 9.2 Hz, 1H), 2.45 (s, 3H), 2.11-1.94 (m, 2H), 1.91-1.58 (m, 6H), 1.49-0.99 (m, 14H), 0.97 (d, J=6.8 Hz, 3H), 0.93-0.80 (m, 12H), 0.76 (s, 3H), 0.70-0.61 (m, 1H).

### Synthesis of B-15.6

To a solution of B-15.5 (140 mg, 0.26 mmol) in DMF (3 mL) was added KI (209 mg, 1.26 mmol) at 25° C. The mixture was stirred at 60° C. for 1 hour. The mixture was poured into water (50 mL) and stirred for 10 mins. The aqueous phase was extracted with EtOAc (3×30 mL) and the combined organic phase was washed with brine (2×100 mL), dried over anhydrous Na₂SO₄, filtered, and concentrated. The residue was purified by silica gel chromatography (0˜10% PE/EtOAc) to provide B-15.6 (140 mg). ¹H NMR (400 MHz, CDCl₃) δ_{H} 3.33 (dd, J=2.0, 9.2 Hz, 1H), 3.17 (dd, J=5.2, 9.6 Hz, 1H), 2.34-2.18 (m, 1H), 2.15-2.05 (m, 1H), 1.92-1.80 (m, 2H), 1.66-1.30 (m, 12H), 1.22-1.06 (m, 6H), 1.01 (d, J=6.0 Hz, 3H), 0.99-0.84 (m, 15H), 0.64-0.73 (m, 1H).

### Synthesis of B-15.7

To a solution of B-15.6 (1.03 g, 2.12 mmol in 10 mL DMF) was added sodium benzenesulfonate (1.04 g, 6.36 mmol) and the mixture was stirred at 40° C. for 1 hour. The reaction mixture was poured into water (100 mL) and the mixture was filtered. The filter cake was washed with water (50 mL). The washed filter cake was dissolved in DCM (100 mL), washed with brine (100 mL), dried over Na₂SO₄, filtered, and concentrated. Purification by flash column chromatography (15˜30% EtOAc in PE) to give B-15.7 (750 mg, 71%). 1H NMR (400 MHz, CDCl₃) δ_{H} 7.97-7.88 (m, 2H), 7.69-7.62 (m, 1H), 7.61-7.53 (m, 2H), 3.15 (d, J=14.0 Hz, 1H), 2.84 (dd, J=9.6, 14.0 Hz, 1H), 2.18-2.06 (m, 2H), 2.03-1.93 (m, 1H), 1.88-1.79 (m, 2H), 1.60-1.25 (m, 12H), 1.19 (d, J=6.4 Hz, 3H), 1.15-0.90 (m, 6H), 0.89-0.81 (m, 10H), 0.77 (s, 3H), 0.75-0.59 (m, 2H).

### Synthesis of B-16.1

To a solution of B-15.7 (260 mg, 0.52 mmol) in THF (4 mL) was added n-BuLi (0.83 mL, 2.07 mmol, 2.5M in hexane) dropwise at -70° C. under N₂. The reaction mixture was stirred at -70° C. for 0.5 hour. DIPA (0.29 mL, 2.07 mmol) was added dropwise at −70° C. for 0.5 hour. A solution of (2R)-1,1,1-trifluoro-2-methyl-3-[(4-methylbenzenesulfonyl)oxy]propan-2-ol (201 mg, 0.67 mmol) in THF (1 mL) was added dropwise at −70° C. The reaction mixture was warmed to 25° C. slowly and stirred for 18 hours. The reaction mixture was quenched with saturated NH₄Cl (20 mL) and extracted with EtOAc (2×10 mL). The combined organic phase was washed with 10% NH₄Cl (30 mL), dried over Na₂SO₄, filtered, and concentrated to give B-16.1 (400 mg).

### Synthesis of B-16

To a solution of B-16.1 (400 mg, 0.64 mmol) in methanol (20 mL) and THF (10 mL) was added nickel (II) chloride (16.4 mg, 0.13 mmol) and Mg powder (612 mg, 25.5 mmol) in four portions under N₂ at 65° C. and the mixture was stirred for 20 mins. The reaction mixture was quenched with citric acid (100 mL, 10%) which was added dropwise until solid was dissolved. The mixture was extracted with EtOAc (3×50 mL) and the organic layer was washed with sat. NaHCO₃ (100 mL), brine (100 mL), dried over Na₂SO₄, filtered, and concentrated. Purification by flash column chromatography (0˜20% of EtOAc in PE) and further purification from MeCN/H₂O (10 mL/4 mL) provided B-16 (26.7 mg, 30%). 1H NMR (400 MHz, CDCl₃) δ_{H} 2.32-2.17 (m, 1H), 2.07-2.00 (m, 1H), 1.92-1.16 (m, 6H), 1.53-1.35 (m, 8H), 1.33 (s, 3H), 1.25-0.96 (m, 12H), 0.94-0.86 (m, 11H), 0.85 (s, 3H), 0.81 (s, 3H), 0.73-0.62 (m, 1H) ¹⁹F NMR (376 MHz, CDCl₃) δ_{F} −82.80 (s) LC-ELSD/MS purity >99%, MS ESI calcd. for C₂₉H₄₉F₃O₂ [M−H₂O+H]⁺ 469.4, found 469.4.

### Synthesis of B-15.8

To a solution of B-15.7 (350 mg, 0.70 mmol) in THF (4 mL) was added n-BuLi (1.11 mL, 2.79 mmol, 2.5M in hexane) dropwise under N₂ at −70° C. The reaction mixture was stirred at -70° C. for 0.5 hour. DIPA (0.39 mL, 2.79 mmol) was added dropwise at −70° C. for 0.5 hour. A solution of (2S)-1,1,1-trifluoro-2-methyl-3-[(4-methylbenzenesulfonyl)oxy]propan-2-ol (270 mg, 0.91 mmol) in THF (1 mL) was added dropwise at −70° C. The reaction mixture was warmed to 25° C. slowly and stirred for 18 hours. The reaction mixture was quenched with saturated NH₄Cl (20 mL) and extracted with EtOAc (2×10 mL). The combined organic phase was washed with 10% NH₄Cl (30 mL), dried over Na₂SO₄, filtered, and concentrated to give B-15.8 (430 mg).

### Synthesis of B-15

To a solution of B-15.8 (430 mg, 0.69 mmol) in methanol (20 mL) and THF (10 mL) was added nickel (II) chloride (17.6 mg, 0.14 mmol) and Mg powder (657 mg, 27.4 mmol) in four portions under N₂ at 65° C. and the mixture was stirred for 20 mins. The reaction mixture was quenched by citric acid (100 mL, 10%) which was added dropwise until solid was dissolved. The mixture was extracted with EtOAc (3×50 mL) and the organic layer was washed with sat. NaHCO₃ (100 mL), brine (100 mL), dried over Na₂SO₄, filtered, and concentrated. Purification by flash column chromatography (0˜20% of EtOAc in PE) and further purification from MeCN/H₂O (10 mL/4 mL) provided B-15 (44.5 mg, 34%). 1H NMR (400 MHz, CDCl₃) δ_{H} 2.30-2.15 (m, 1H), 2.07-1.99 (m, 1H), 1.93-1.83 (m, 2H), 1.74-1.57 (m, 4H), 1.54-1.35 (m, 7H), 1.32 (s, 3H), 1.29-0.95 (m, 14H), 0.94-0.86 (m, 10H), 0.85 (s, 3H), 0.81 (s, 3H), 0.73-0.61 (m, 1H). ¹⁹F NMR (376 MHz, CDCl₃) δ_{F} −83.12 (s) LC-ELSD/MS purity >99%, MS ESI calcd. for C₂₉H₄₉F₃O₂ [M−H₂O+H]⁺ 469.4, found 469.4.

### Example 27: Synthesis of (3S,5S,8R,9S,10S,13R,14S,15S,17R)-15-cyclopropyl-3-ethyl-17-((R)-6-hydroxy-6-methylheptan-2-yl)-10,13-dimethylhexadecahydro-1H-cyclopenta[a]phenanthren-3-ol (B-17)

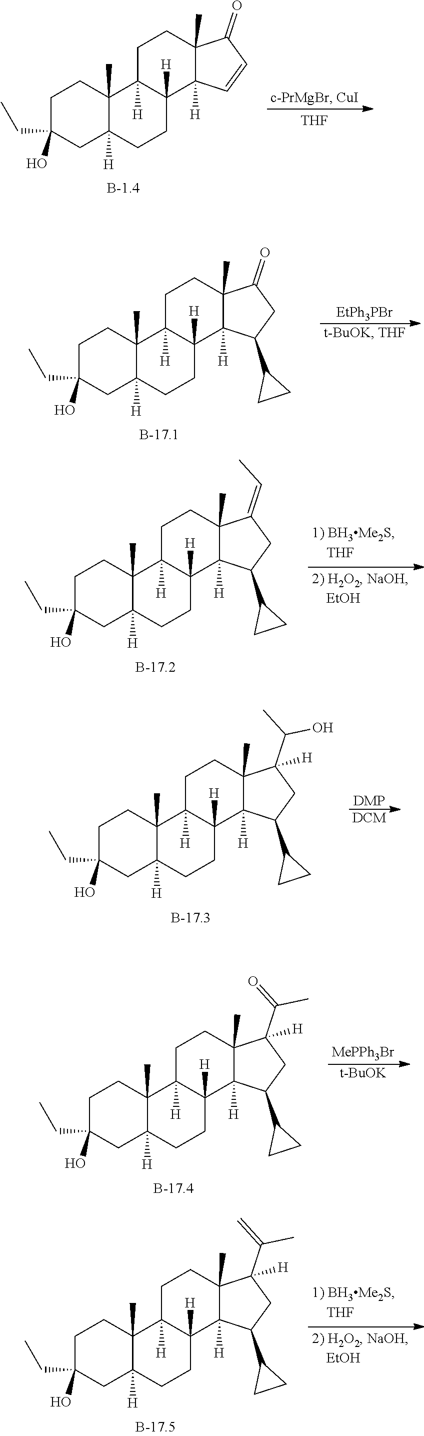

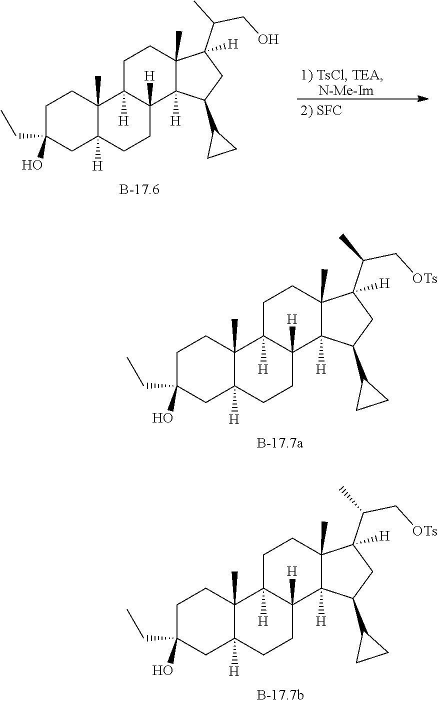

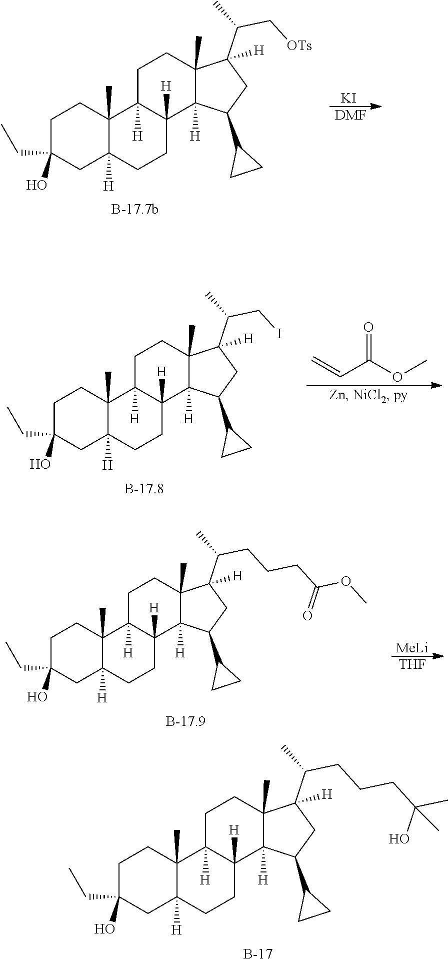

### Synthesis of B-17.1

To a solution of c-PrMgBr (125 mL, 62.7 mmol, 0.5M) in THF (150 mL) was added CuI (8.95 g, 47.0 mmol) at 0° C. and the mixture was stirred for 1 hour. The mixture was reacted with B-1.4 (5.00 g, 15.7 mmol) in THF (50 mL) was added at 0° C. and the mixture was stirred for 3 hours. The mixture was poured into saturated NH₄Cl (100 mL) and extracted with EtOAc (3×100 mL). The combined organic layer was washed with brine (2×50 mL), dried over anhydrous Na₂SO₄, filtered, and concentrated. The residue was purified by flash column chromatography (0%˜30% of EtOAc in PE) to provide B-17.1 (5.00 g, 89%). 1H NMR (400 MHz, CDCl₃) δ_{H} 2.48-2.24 (m, 2H), 2.21-2.10 (m, 1H), 1.97-1.86 (m, 1H), 1.84-1.72 (m, 2H), 1.67-1.59 (m, 5H), 1.55-1.43 (m, 2H), 1.41-1.19 (m, 9H), 1.09-0.92 (m, 5H), 0.90 (s, 3H), 0.85-0.89 (m, 3H), 0.79-0.65 (m, 2H), 0.52-0.40 (m, 1H), 0.26-0.16 (m, 1H), 0.14-0.02 (m, 1H).

### Synthesis of B-17.2

To a mixture of EtPPh₃Br (15.4 g, 41.7 mmol) in THF (100 mL) was added t-BuOK (4.67 g, 41.7 mmol) at 20° C. under N₂. The resulting mixture was stirred at 50° C. for 30 mins. B-17.1 (5.00 g, 13.9 mmol) in THF (50 mL) was added in portions below 50° C. The reaction mixture was stirred at 45° C. for 16 hours. The reaction mixture was quenched with 10% NH₄Cl aqueous (200 mL) at 15° C. and the THF layer was separated. The aqueous layer was extracted with EtOAc (3×150 mL) and the combined organic phase was washed with brine (2×100 mL), filtered, and concentrated. The residue was purified by flash column chromatography (0%˜30% of EtOAc in PE) to give B-17.2 (4.20 g, 82%). ¹H NMR (400 MHz, CDCl₃) δ_{H} 5.19-5.02 (m, 1H), 2.46-2.33 (m, 1H), 2.30-2.15 (m, 2H), 2.12-2.05 (m, 1H), 1.85-1.72 (m, 1H), 1.68-1.60 (m, 5H), 1.57-1.52 (m, 3H), 1.48-1.20 (m, 10H), 1.19-1.05 (m, 4H), 1.04-0.92 (m, 2H), 0.91-0.86 (m, 6H), 0.84-0.68 (m, 2H), 0.62-0.48 (m, 1H), 0.42-0.29 (m, 1H), 0.19-0.03 (m, 2H).

### Synthesis of B-17.3

To a solution of B-17.2 (2.70 g, 7.28 mmol) in THF (30 mL) was added BH₃·Me₂S (2.91 mL, 29.1 mmol, 10 M) and the mixture was stirred at 25° C. for 16 hours. Ethanol (7.28 mL, 72.8 mmol, 10 M) was added at 15° C., followed by NaOH (14.5 mL, 5.0 M, 72.8 mmol) at 0° C. Hydrogen peroxide (7.28 mL, 10 M, 72.8 mmol) was added dropwise at 0° C. The mixture was warmed and stirred at 70° C. for 1 hour. The mixture was cooled to 15° C. and Na₂S₂O₃ (100 mL, sat.) was added. The aqueous layer was extracted with EtOAc (3×100 mL) and the combined organic layers were washed with brine (2×50 mL), dried over Na₂SO₄, filtered, and concentrated to give B-17.3 (3.50 g). ¹H NMR (400 MHz, CDCl₃) δ_{H} 3.82-3.68 (m, 1H), 3.82-3.68 (m, 2H), 2.31-2.10 (m, 2H), 1.87-1.68 (m, 2H), 1.54-1.32 (m, 5H), 1.30-1.20 (m, 10H), 1.17-0.95 (m, 6H), 0.92-0.84 (m, 10H), 0.80-0.64 (m, 2H), 0.63-0.53 (m, 1H), 0.41-0.30 (m, 1H), 0.15-0.08 (m, 1H), −0.05-0.07 (m, 1H).

### Synthesis of B-17.4

To a solution of B-17.3 (1.50 g, 3.85 mmol) in DCM (20 mL) was added DMP (3.26 g, 7.70 mmol) at 25° C. and the reaction mixture was stirred for 10 mins. The mixture was quenched with saturated NaHCO₃/Na₂S₂O₃ (1:1, 100 mL) at 10° C. The mixture was extracted with DCM (3×100 mL). The combined organic phase was washed with brine (2×50 mL), dried over anhydrous Na₂SO₄, filtered, and concentrated. The residue was purified by flash column chromatography (0˜25% ethyl acetate in PE) to give B-17.4 (2.00 g). ¹H NMR (400 MHz, CDCl₃) δ_{H} 2.45-2.35 (m, 1H), 2.29-2.20 (m, 1H), 2.12 (s, 3H), 1.99-1.91 (m, 2H), 1.81-1.71 (m, 1H), 1.66-1.58 (m, 5H), 1.47-0.93 (m, 12H), 0.92-0.85 (m, 8H), 0.82 (s, 3H), 0.81-0.69 (m, 2H), 0.61-0.55 (m, 1H), 0.44-0.38 (m, 1H), 0.15-0.09 (m, 1H), 0.07-0.01 (m, 1H).

### Synthesis of B-17.5

To a mixture of MePPh₃Br (3.67 g, 10.3 mmol) in THF (50 mL) was added t-BuOK (1.15 g, 10.3 mmol) at 20° C. under N₂ and the mixture was stirred at 50° C. for 30 mins. B-17.4 (1.00 g, 2.58 mmol) in THF (15 mL) was added in portions below 50° C. The reaction mixture was stirred at 45° C. for 16 hours. The reaction mixture was quenched with 10% aqueous NH₄Cl (200 mL) at 15° C. and the THF layer was separated. The aqueous layer was extracted with EtOAc (3×150 mL). The combined organic phase was washed with brine (2×100 mL), filtered, and concentrated. The residue was purified by flash column chromatography (0˜30% of EtOAc in PE) to give B-17.5 (990 mg, 100%). ¹H NMR (400 MHz, CDCl₃) δ_{H} 4.85 (s, 1H), 4.73 (s, 1H), 2.37-2.18 (m, 1H), 2.02-1.80 (m, 3H), 1.76 (s, 3H), 1.66-1.50 (m, 6H), 1.49-1.35 (m, 6H), 1.34-1.18 (m, 6H), 1.19-1.01 (m, 6H), 0.98-0.94 (m, 3H), 0.81-0.76 (m, 3H), 0.74-0.64 (m, 1H), 0.64-0.52 (m, 1H), 0.45-0.32 (m, 1H), 0.18-0.08 (m, 1H), 0.07-0.03 (m, 1H).

### Synthesis of B-17.6

To a solution of B-17.5 (990 mg, 2.57 mmol) in THF (30 mL) was added BH₃·Me₂S (1.01 mL, 10.2 mmol, 10 M) and the mixture was stirred at 25° C. for 16 hours. To the resulting mixture was added ethanol (2.56 mL, 25.6 mmol, 10 M) at 15° C., followed by NaOH (5.12 mL, 5.0 M, 25.6 mmol) at 0° C. Hydrogen peroxide (2.56 mL, 10M, 25.6 mmol) was added dropwise at 0° C. The reaction mixture was stirred at 70° C. for 1 hour. The mixture was cooled to 15° C. and Na₂S₂O₃ (200 mL, sat.) was added. The aqueous layer was extracted with EtOAc (150 mL×3). The combined organic layer was washed with brine (2×10 mL), dried over Na₂SO₄, filtered, and concentrated to give B-17.6 (1.00 g). ¹H NMR (400 MHz, CDCl₃) δ_{H} 3.75-3.59 (m, 1H), 3.53-3.34 (m, 1H), 2.27-2.07 (m, 2H), 1.98-1.68 (m, 3H), 1.56-1.43 (m, 4H), 1.40-1.27 (m, 6H), 1.21-1.08 (m, 6H), 1.07-1.02 (m, 2H), 0.98-0.93 (m, 2H), 0.91-0.89 (m, 1H), 0.89-0.86 (m, 9H), 0.85-0.63 (m, 5H), 0.62-0.51 (m, 1H), 0.40-0.29 (m, 1H), 0.15-0.07 (m, 1H), 0.03-0.09 (m, 1H).

### Synthesis of B-17.7a & B-17.7b

To a solution of B-17.6 (1.00 g, 2.48 mmol) in DCM (15 mL) was added N-Me-Im (814 mg, 9.92 mmol), Et₃N (250 mg, 2.48 mmol), and TsCl (709 mg, 3.72 mmol) and the mixture was stirred at 20° C. for 20 mins. Water (10 mL) was added and the mixture was washed with HCl (2×20 mL), NaHCO₃ (2×20 mL) and brine (2×20 mL), dried over Na₂SO₄, filtered, and concentrated. The residue was purified by flash column chromatography (0˜30% ethyl acetate in PE) and further purified by SFC (column: DAICEL CHIRALPAK AD-H (250 mm*30 mm, 5 um); Mobile phase: A: CO₂ B: 0.1% NH₃H₂O IPA; gradient: from 30% to 30% of B, Flow Rate (ml/min): 60) to give B-17.7a (460 mg, 46%) and B-17.7b (330 mg, 33%).

B-17.7a: ¹H NMR (400 MHz, CDCl₃) δ_{H} 7.78 (d, J-8.3 Hz, 2H), 7.34 (d, J=7.9 Hz, 2H), 4.11-4.08 (m, 1H), 3.81 (dd, J=7.3, 9.3 Hz, 1H), 2.45 (s, 3H), 2.26-2.15 (m, 1H), 1.82-1.56 (m, 8H), 1.46-1.30 (m, 5H), 1.23-1.19 (m, 2H), 1.18-0.93 (m, 7H), 0.91-0.87 (m, 6H), 0.86-0.79 (m, 4H), 0.78 (s, 3H), 0.77-0.50 (m, 4H), 0.37-0.28 (m, 1H), 0.12-0.06 (m, 1H), −0.01-0.10 (m, 1H)

B-17.7b: ¹H NMR (400 MHz, CDCl₃) δ_{H} 7.78 (d, J=8.4 Hz, 2H), 7.34 (d, J=8.0 Hz, 2H), 4.00-3.93 (m, 1H), 3.86-3.78 (m, 1H), 2.45 (s, 3H), 2.27-2.10 (m, 1H), 1.99-1.80 (m, 2H), 1.75-1.61 (m, 3H), 1.60-1.32 (m, 8H), 1.31-1.01 (m, 12H), 1.00-0.93 (m, 4H), 0.91-0.86 (m, 4H), 0.85 (s, 3H), 0.81 (s, 3H), 0.78-0.51 (m, 4H), 0.34-0.26 (m, 1H), 0.12-0.04 (m, 1H), −0.05-0.17 (m, 1H).

### Synthesis of B-17.8

To a solution of B-17.7b (100 mg, 0.17 mmol) in DMF (5 mL) was added KI (148 mg, 0.89 mmol) at 25° C. The mixture was stirred at 60° C. for 1 hour and then poured into water (30 mL) and stirred for 20 mins. The aqueous phase was extracted with EtOAc (3×50 mL). The combined organic phase was washed with brine (2×20 mL), dried over anhydrous Na₂SO₄, filtered, and concentrated. The residue was purified by flash column chromatography (0-20% of EtOAc in PE) to give B-17.8 (70.0 mg, 76%). ¹H NMR (400 MHz, CDCl₃) δ_{H} 3.38-3.29 (m, 1H), 3.25-3.12 (m, 1H), 2.26-2.11 (m, 2H), 1.93-1.85 (m, 1H), 1.78-1.60 (m, 3H), 1.48-1.35 (m, 4H), 1.34-1.23 (m, 7H), 1.22-1.07 (m, 6H), 1.04-1.00 (m, 3H), 0.91-0.88 (m, 6H), 0.87-0.86 (m, 4H), 0.83-0.64 (m, 3H), 0.61-0.55 (m, 1H), 0.38-0.30 (m, 1H), 0.16-0.08 (m, 1H), 0.00-0.07 (m, 1H).

### Synthesis of B-17.9

To a mixture of Zn powder (62.4 mg, 0.95 mmol) in dry pyridine (1 mL) was added slowly methyl acrylate (82.2 mg, 0.95 mmol) and NiCl₂₆H₂O (26.5 mg, 0.20 mmol) at 20° C. under N₂. After stirring at 60° C. for 2 hours, the mixture was cooled to 20° C. and a solution of B-17.8 (70.0 mg, 0.13 mmol) in pyridine (1 mL) was added slowly. The reaction mixture was stirred for 1.5 hours at 20° C. and then poured into NH₄Cl (20 mL). The mixture was filtered through a pad of silica gel. The filtrate was extracted with EtOAc (2×20 mL) and the organic phase was dried over Na₂SO₄, filtered, and concentrated. The residue was purified by flash column chromatography (0-20% of EtOAc in PE) to give B-17.9 (60.0 mg). ¹H NMR (400 MHz, CDCl₃) δ_{H} 3.67 (s, 3H), 2.31-2.10 (m, 4H), 1.95-1.90 (m, 1H), 1.78-1.65 (m, 3H), 1.41-1.35 (m, 4H), 1.29-1.21 (m, 8H), 1.14-0.97 (m, 8H), 0.95-0.90 (m, 5H), 0.89-0.85 (m, 10H), 0.72-0.50 (m, 3H), 0.40-0.28 (m, 1H), 0.15-0.07 (m, 1H), 0.00-0.07 (m, 1H).

### Synthesis of B-17

To a solution of B-17.9 (60.0 mg, 0.12 mmol) in THF (5 ml) was added methyl lithium (0.787 mL, 1.26 mmol, 1.6 M in Et₂O) dropwise at 0° C. under N₂. The reaction mixture was stirred at 20° C. for 2 hours. The reaction mixture was quenched with saturated NH₄Cl (30 mL) and extracted with ethyl acetate (3×30 mL). The combined organic phase was washed with brine (2×30 mL), dried over anhydrous Na₂SO₄, filtered, and concentrated. The residue was purified by flash column chromatography (0˜25% of EtOAc in PE) to give B-17 (21.3 mg, 36%). ¹H NMR (400 MHz, CDCl₃) δ_{H} 2.27-2.04 (m, 2H), 1.98-1.88 (m, 1H), 1.80-1.58 (m, 5H), 1.55-1.33 (m, 10H), 1.31-1.23 (m, 5H), 1.22-1.20 (m, 7H), 1.18-0.96 (m, 7H), 0.95-0.91 (m, 3H), 0.91-0.87 (m, 3H), 0.87-0.85 (m, 6H), 0.84-0.74 (m, 2H), 0.71-0.64 (m, 1H), 0.60-0.51 (m, 1H), 0.38-0.28 (m, 1H), 0.13-0.05 (m, 1H), 0.01-0.08 (m, 1H). LC-ELSD/MS purity >99%, MS ESI calcd. for C32H₅₆₀₂ [M−2H₂O+H]⁺ 437.4, found 437.4.

### Example 28: Synthesis of (3S,5S,8R,9S,10S,13R,14S,15R,17R)-3-ethyl-17-((R)-6-hydroxy-6-methylheptan-2-yl)-10,13,15-trimethylhexadecahydro-1H-cyclopenta[a]phenanthren-3-ol (B-18)

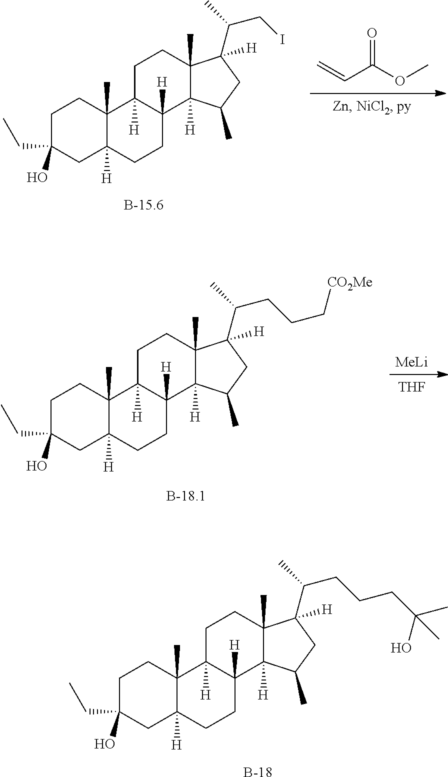

### Synthesis of B-18.1

To a mixture of Zn powder (131 mg, 2.01 mmol) in dry pyridine (2 mL) was added slowly methyl prop-2-enoate (173 mg, 2.01 mmol) and NiCl₂·6H₂O (71.6 mg, 0.43 mmol) at 20° C. under N₂. After stirring at 60° C. for 2 hours, the mixture was cooled to 20° C. and a solution of B-15.6 (140 mg, 0.29 mmol) in pyridine (1 mL) was added slowly. The reaction mixture was stirred for 1.5 hours at 20° C. and then diluted with NH₄Cl (20 mL). The mixture was filtered through a pad of silica gel. The filtrate was extracted with EtOAc (2×20 mL) and the organic phase was dried over Na₂SO₄, filtered, and concentrated. The residue was purified by flash column chromatography (0-20% of EtOAc in PE) to give B-18.1 (70.0 mg, 55%). ¹H NMR (400 MHz, CDCl₃) δ_{H} 3.67 (s, 3H), 2.34-2.18 (m, 3H), 2.06-1.63 (m, 5H), 1.50-1.19 (m, 12H), 1.17-0.95 (m, 8H), 0.93-0.86 (m, 12H), 0.85 (s, 3H), 0.80 (s, 3H), 0.73-0.61 (m, 1H) LC-ELSD/MS purity 98.9%, MS ESI calcd. for C₂₉H₅₀O₃ [M−H₂O+H]⁺ 429.4, found 429.4.

### Synthesis of B-18

To a solution of B-18.1 (55.0 mg, 0.12 mmol) in THF (2 mL) was added methyl lithium (0.77 mL, 1.23 mmol, 1.6 M in Et₂O) dropwise at 0° C. under N₂. The reaction mixture was stirred at 20° C. for 2 h. The reaction mixture was quenched with saturated NH₄Cl (30 mL) and extracted with ethyl acetate (3×30 mL). The combined organic phase was washed with brine (2×100 mL), dried over anhydrous Na₂SO₄, filtered, and concentrated. The residue was purified by flash column chromatography (0˜25% of EtOAc in PE) to give B-18 (25.8 mg, 47%). ¹H NMR (400 MHz, CDCl₃) δ_{H} 2.31-2.16 (m, 1H), 2.11-1.96 (m, 1H), 1.93-1.83 (m, 2H), 1.65-1.44 (m, 10H), 1.41-1.24 (m, 9H), 1.21 (s, 6H), 1.19-0.94 (m, 8H), 0.93-0.86 (m, 10H), 0.85 (s, 3H), 0.80 (s, 3H), 0.72-0.60 (m, 1H) LC-ELSD/MS purity >99%, MS ESI calcd. for C₃₀H₅₄O₂ [M−2H₂O+H]⁺ 411.4, found 411.4.

### Example 29: Synthesis of (3S,5S,8R,9S,10S,13R,14S,15R,17R)-15-cyclopropyl-3-ethyl-17-((R)-6-hydroxy-6-methylheptan-2-yl)-10,13-dimethylhexadecahydro-1H-cyclopenta[a]phenanthren-3-ol (B-19)

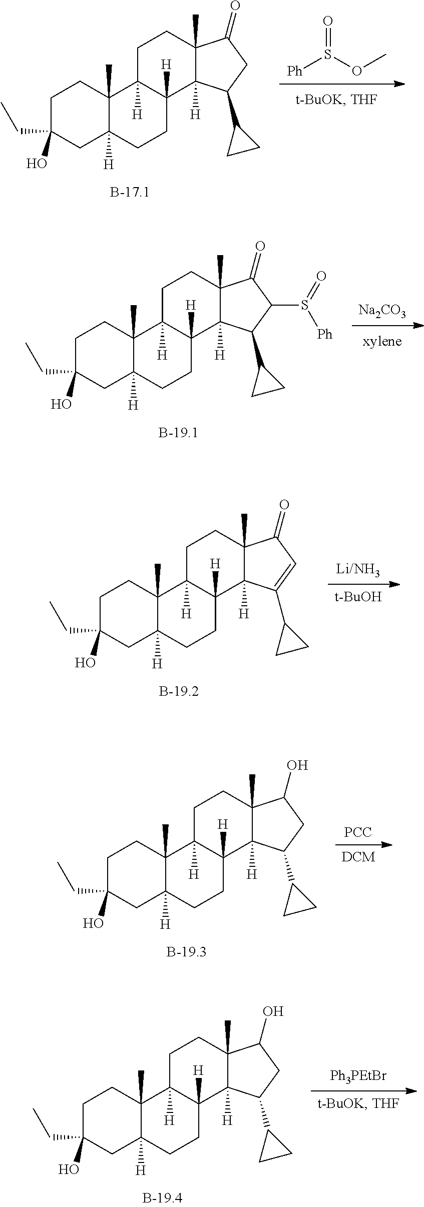

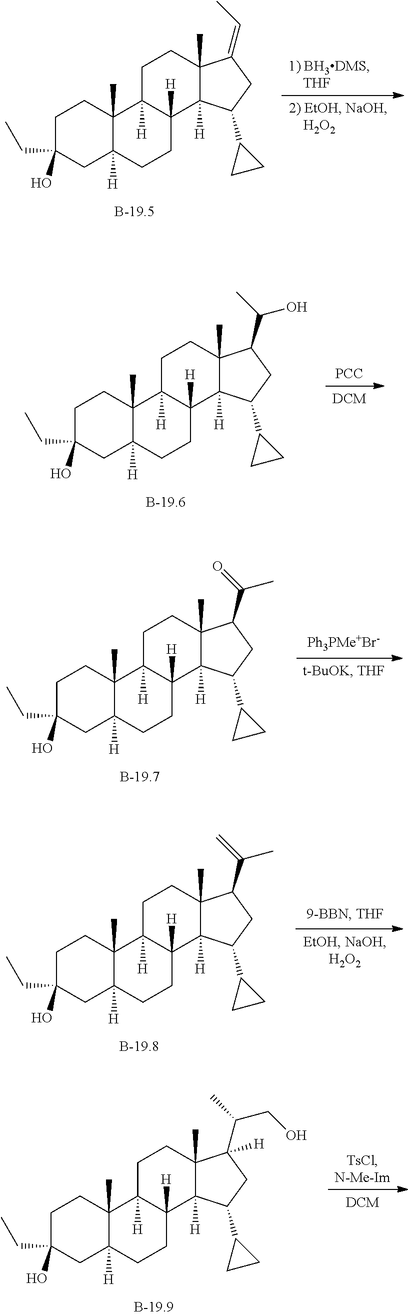

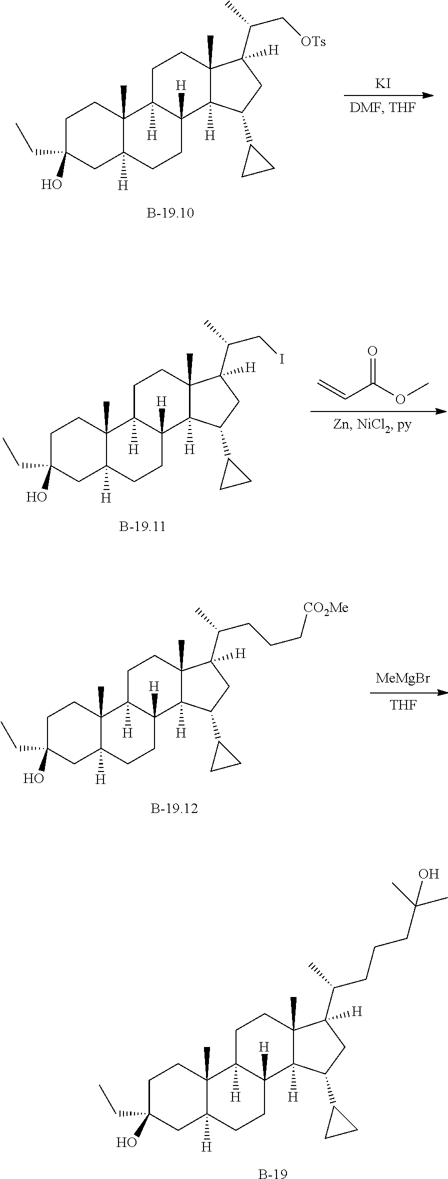

### Synthesis of B-19.1

To a solution of t-BuOK (2.24 g, 20.0 mmol) in THF (100 mL) was added B-17.1 (3.60 g, 10.0 mmol) at 20° C. under N₂ and the mixture was stirred for 10 mins. Methyl benzenesulfinate (3.12 g, 20.0 mmol) was added and the mixture was stirred at 30° C. for 0.5 hour. The mixture was quenched with NH₄Cl (50 mL) and extracted with EtOAc (50×3 mL). The organic layer was separated, dried over Na₂SO₄, filtered, and concentrated to give B-19.1 (5.20 g).

### Synthesis of B-19.2

To a mixture of B-19.1 (3.40 g, 7.04 mmol) in xylene (70 mL) was added Na₂CO₃ (11.1 mL, 105 mmol) and the mixture was stirred at 125° C. for 16 hours under N₂. The mixture was concentrated and purified by flash column chromatography (0˜30% of EtOAc in PE) to give B-19.2 (1.10 g, 44%). ¹H NMR (400 MHz, CDCl₃) δ_{H} 5.49-5.44 (m, 1H), 2.51-2.42 (m, 1H), 2.37-2.30 (m, 1H), 1.93-1.72 (m, 2H), 1.43-1.10 (m, 13H), 1.09-0.99 (m, 5H), 0.98-0.93 (m, 2H), 0.92-0.79 (m, 10H), 0.59-0.49 (m, 1H).

### Synthesis of B-19.3

Lithium (233 mg, 33.6 mmol) was added to freshly prepared liquid ammonia (70 mL) in portions at −70° C. and then stirred at -70° C. for 1 hour. A solution of B-19.2 (2.40 g, 6.73 mmol) and a solution of t-butanol (1.99 g, 26.9 mmol) in dry THF (70 mL) were added to the mixture and the temperature was maintained below-60° C. Stirring was continued at 70° C. for 1 hour. NH₄Cl (1.79 g) was added and the mixture was warmed to 20° C. and stirred for 16 hours. The reaction mixture was diluted with H₂O (50 mL) and extracted with EtOAc (3×50 mL). The combined organic layers were washed with brine (100 mL), dried over anhydrous Na₂SO₄, filtered, and concentrated to give B-19.3 (2.00 g).

### Synthesis of B-19.4

To a solution of B-19.3 (1.00 g, 2.77 mmol) in DCM (10 mL) was added silica gel (1.00 g) and PCC (1.19 g, 5.54 mmol) at 20° C. and the mixture was stirred for 2 hours. The mixture was added to PE (5 mL) and concentrated. Purification by flash column chromatography (0˜30% of EtOAc in PE) and further purification from MeCN/water (20 mL, 1:1) to give B-19.4 (290 mg, 29%). ¹H NMR (400 MHz, CDCl₃) δ_{H} 2.61 (dd, J=8.4 Hz, 19.6 Hz, 1H) 2.39-2.31 (m, 1H), 1.97-1.85 (m, 1H), 1.80-1.62 (m, 4H), 1.58-1.09 (m, 15H), 1.04-0.95 (m, 1H), 0.91-0.84 (m, 9H), 0.79-0.65 (m, 2H), 0.61-0.48 (m, 2H), 0.27-0.19 (m, 1H), 0.15-0.07 (m, 1H). LC-ELSD/MS purity >99%, MS ESI calcd. for C₂₄H₃₈O₂ [M−H₂O+H]⁺ 341.3, found 341.3.

### Synthesis of B-19.5

To a mixture of EtPPh₃Br (835 mg, 2.25 mmol) in THF (4 mL) was added t-BuOK (252 mg, 2.25 mmol) at 20° C. under N₂. The resulting mixture was stirred at 45° C. for 30 mins. B-19.4 (270 mg, 0.75 mmol) in THF (2 mL) was added in portions below 45° C. and the mixture was stirred for 16 hours. The reaction mixture was quenched with 10% aqueous NH₄Cl (10 mL), extracted with EtOAc (2×10 mL), washed with brine (10 mL), dried over Na₂SO₄, filtered, and concentrated. The residue was purified by flash column chromatography (0˜15% of EtOAc in PE) to give B-19.5 (180 mg, 65%). ¹H NMR (400 MHz, CDCl₃) δ_{H} 5.15-5.03 (m, 1H), 2.57-2.45 (m, 1H), 2.36-2.29 (m, 1H), 2.26-2.17 (m, 1H), 2.06-1.97 (m, 1H), 1.67-1.59 (m, 6H), 1.52-0.94 (m, 15H), 0.92-0.83 (m, 10H), 0.79-0.70 (m, 1H), 0.63-0.53 (m, 1H), 0.51-0.38 (m, 2H), 0.19-0.04 (m, 2H).

### Synthesis of B-19.6

To a solution of B-19.5 (180 mg, 0.49 mmol) in THF (5 mL) was added BH₃·DMS (1.01 mL, 10 M, 10.1 mmol) and the mixture was stirred at 20° C. for 16 hours. To the resulting mixture was added ethanol (0.342 mL, 5.82 mmol) at 0° C. over 0.5 hour followed by addition of NaOH (1.16 mL, 5.0 M, 5.82 mmol) at 0° C. H₂O₂ (0.58 mL, 10 M, 5.82 mmol) was added and the reaction mixture was stirred at 60° C. for 1 hour. The mixture was cooled to 0° C. and Na₂SO₃ (10 mL, 15%) was added followed by extraction with EtOAc (3×20 mL). The organic layer was washed with brine (20 mL), dried over anhydrous Na₂SO₄, filtered, and concentrated to give B-19.6 (210 mg). ¹H NMR (400 MHz, CDCl₃) δ_{H} 3.74-3.63 (m, 1H), 2.19-2.09 (m, 1H), 1.84-1.80 (m, 1H), 1.40-1.32 (m, 5H), 1.31-1.25 (m, 5H), 1.21 (d, J=6.4 Hz, 3H), 1.17-0.93 (m, 10H), 0.88 (t, J=7.6 Hz, 3H), 0.84 (s, 3H), 0.83-0.79 (m, 1H), 0.77-0.62 (m, 6H), 0.60-0.38 (m, 3H), 0.20-0.08 (m, 2H).

### Synthesis of B-19.7

To a solution of B-19.6 (210 mg, 0.54 mmol) in DCM (3 mL) was added silica gel (250 mg) and PCC (232 mg, 1.08 mmol) at 20° C. and the mixture was stirred for 2 hours. The mixture was diluted with PE (2 mL) and concentrated. The residue was purified by flash column chromatography (0˜30% of EtOAc in PE) to give B-19.7 (86.8 mg, 41%). ¹H NMR (400 MHz, CDCl₃) δ_{H} 2.53 (t, J=9.6 Hz, 1H), 2.40-2.30 (m, 1H), 2.22-2.13 (m, 1H), 2.11 (s, 3H), 1.98-1.92 (m, 1H), 1.58-1.35 (m, 10H), 1.34-0.95 (m, 10H), 0.88 (t, J=7.2 Hz, 3H), 0.84 (s, 3H), 0.78-0.71 (m, 1H), 0.69-0.63 (m, 1H), 0.62 (s, 3H), 0.52-0.39 (m, 2H), 0.19-0.08 (m, 2H). LC-ELSD/MS purity >99%, MS ESI calcd. for C₂₋₆H₄₂₀₂ [M−H₂O+H]⁺ 369.3, found 369.3.

### Synthesis of B-19.8

To a mixture of MePPh₃Br (12.4 g, 34.8 mmol) in THF (50 mL) was added t-BuOK (3.90 g, 34.8 mmol) at 20° C. under N₂ and the mixture was warmed and stirred at 55° C. for 30 minutes. B-19.7 (4.5 g, 11.6 mmol) in THF (50 mL) was added in portions below 50° C. and the reaction mixture was stirred at 55° C. for 3 hours. The reaction mixture was quenched with 10% NH₄Cl (50 mL) and extracted with EtOAc (2×50 mL). The organic layers were combined, washed with brine (50 mL), dried over Na₂SO₄, filtered, and concentrated. Trituration with MeOH/H₂O (200 mL, 1:1) provided B-19.8 (3.90 g, 87%). ¹H NMR (400 MHz, CDCl₃) δ_{H} 4.83 (s, 1H), 4.66 (s, 1H), 2.12-2.06 (m, 1H), 2.03-1.87 (m, 2H), 1.79-1.75 (m, 1H), 1.74 (s, 3H), 1.56-1.31 (m, 9H), 1.31-0.93 (m, 11H), 0.88 (t, J=7.2 Hz, 3H), 0.84 (s, 3H), 0.78-0.65 (m, 2H), 0.57 (s, 3H), 0.53-0.35 (m, 2H), 0.20-0.06 (m, 2H).

### Synthesis of B-19.9

To a solution of B-19.8 (3.50 g, 9.09 mmol) in THF (35 mL) was added 9-BBN dimer (4.41 g, 18.1 mmol) and the mixture was stirred at 20° C. for 16 hours. To the resulting mixture was added ethanol (6.42 mL, 109 mmol) at 0° C. over 0.5 hour followed by NaOH (21.8 mL, 5.0 M, 109 mmol) at 0° C. H₂O₂ (10.9 mL, 10 M, 109 mmol) was added and the reaction mixture was stirred at 60° C. for 1 hour. The mixture was diluted with Na₂SO₃ (120 mL, 15%) and extracted with EtOAc (3×30 mL). The organic layer was washed with saturated brine (20 mL), dried over anhydrous Na₂SO₄, filtered, and concentrated. Trituration with MeOH/H₂O (90 mL, 1:1) followed purification by silica gel chromatography (0˜30% of EtOAc in PE) provided B-19.9 (1.30 g, 37%). ¹H NMR (400 MHz, CDCl₃) δ_{H} 3.65-3.59 (m, 1H), 3.40-3.34 (m, 1H), 2.18-2.08 (m, 1H), 1.96-1.89 (m, 1H), 1.56-1.22 (m, 16H), 1.22-1.06 (m, 5H), 1.03 (d, J=6.4 Hz, 3H), 1.02-0.90 (m, 3H), 0.87 (t, J=7.2 Hz, 3H), 0.83 (s, 3H), 0.74-0.69 (m, 1H), 0.67 (s, 3H), 0.65-0.59 (m, 1H), 0.53-0.35 (m, 2H), 0.18-0.02 (m, 2H).

### Synthesis of B-19.10

To a solution of B-19.9 (1.30 g, 3.22 mmol) in DCM (10 mL) was added N-Me-Im (1.05 mg, 12.8 mmol) and TsCl (1.22 mg, 6.44 mmol) and the mixture was stirred at 20° C. for 2 hours. The mixture was quenched with NaHCO₃ (10 mL) and extracted with DCM (2×20 mL). The combined organic phase was washed with brine (2×20 mL), dried over Na₂SO₄, filtered, and concentrated. Purification by flash column chromatography (0˜40% of EtOAc in PE) and further purification by SFC (Column: DAICEL CHIRALPAK AD (250 mm*50 mm, 10 um); Condition: 0.1% NH₃H₂O ETOH; Begin B: 45%; End B: 45%; Flow Rate (ml/min): 220; Injections: 150) provided B-19.10 (1.00 g, 72%). ¹H NMR (400 MHz, CDCl₃) δ_{H} 7.79 (d, J=8.4 Hz, 2H), 7.35 (d, J=8.4 Hz, 2H), 4.02-3.94 (m, 1H), 3.79-3.70 (m, 1H), 2.45 (s, 3H), 2.15-2.06 (m, 1H), 1.89-1.81 (m, 1H), 1.68-1.58 (m, 3H), 1.51-1.00 (m, 18H), 0.97 (d, J=6.4 Hz, 3H), 0.91-0.85 (m, 5H), 0.82 (s, 3H), 0.73-0.64 (m, 1H), 0.61 (s, 3H), 0.59-0.54 (m, 1H), 0.51-0.44 (m, 1H), 0.39-0.34 (m, 1H), 0.11-0.04 (m, 1H), 0.01-0.06 (m, 1H).

### Synthesis of B-19.11

To a solution of B-19.10 (1.00 g, 1.79 mmol) in DMF (10 mL) was added KI (1.42 g, 8.59 mmol) at 20° C. The mixture was stirred at 60° C. for 16 hours. The reaction mixture was poured into water (50 mL) and extracted with EtOAc (2×40 mL). The combined organic layers were washed with LiCl (3×30 mL, 5%) and brine (20 mL), dried over Na₂SO₄, filtered, and concentrated to give B-19.11 (1.00 g). ¹H NMR (400 MHz, CDCl₃) δ_{H} 3.38-3.29 (m, 1H), 3.19-3.11 (m, 1H), 2.21-2.07 (m, 1H), 1.96-1.79 (m, 3H), 1.51-1.07 (m, 17H), 1.06-0.93 (m, 6H), 0.93-0.86 (m, 4H), 0.83 (s, 3H), 0.75-0.66 (m, 5H), 0.54-0.38 (m, 2H), 0.19-0.06 (m, 2H).

### Synthesis of B-19.12

To a mixture of Zn powder (178 mg, 2.73 mmol) in dry pyridine (2 mL) was added slowly methyl acrylate (235 mg, 2.73 mmol) and NiCl₂ (75.8 mg, 0.585 mmol) at 25° C. under N₂. After stirring at 60° C. for 2 hours, the mixture was cooled to 25° C. and a solution of B-19.11 (200 mg, 0.39 mmol) in dry pyridine (2 mL) was added slowly. The reaction mixture was stirred for 2 hours at 25° C. and then diluted with NH₄Cl (10 mL). The mixture was filtered through a pad of silica gel. The mother liquor was extracted with EtOAc (2×20 mL), washed with brine (20 mL), dried over Na₂SO₄, filtered, and concentrated. The residue was purified by flash column chromatography (0˜20% of EtOAc in PE) to give B-19.12 (130 mg, 71%). ¹H NMR (400 MHz, CDCl₃) δ_{H} 3.67 (s, 3H), 2.33-2.20 (m, 2H), 2.18-2.09 (m, 1H), 1.96-1.88 (m, 1H), 1.75-1.57 (m, 5H), 1.50-1.13 (m, 14H), 1.12-0.95 (m, 7H), 0.93-0.85 (m, 7H), 0.83 (s, 3H), 0.73-0.67 (m, 1H), 0.65 (s, 3H), 0.64-0.57 (m, 1H), 0.51-0.45 (m, 1H), 0.44-0.37 (m, 1H), 0.17-0.11 (m, 1H), 0.10-0.04 (m, 1H). LC-ELSD/MS purity >99%, MS ESI calcd. for C₃₁H₅₂O₃ [M−H₂O+H]⁺ 455.4, found 455.4.

### Synthesis of B-19

A solution of MeMgBr (1.08 mL, 3 M, 3.25 mmol) in THF (2 mL) was reacted with B-19.12 (110 mg, 0.23 mmol) in THF (2 mL) at 20° C. under N₂. The mixture was stirred at 20° C. for 2 hours and then quenched with saturated NH₄Cl (10 mL) and extracted with EA (2×10 mL). The combined organic phase was washed with brine (10 mL), dried over Na₂SO₄, filtered, and concentrated. The residue was purified by silica gel chromatography (0˜20% of EtOAc in PE) to give B-19 (67.2 mg, 61%). ¹H NMR (400 MHz, CDCl₃) δ_{H} 2.18-2.11 (m, 1H), 1.98-1.90 (m, 1H), 1.65-1.58 (m, 3H), 1.53-1.33 (m, 11H), 1.31-1.16 (m, 13H), 1.15-0.94 (m, 8H), 0.93-0.85 (m, 7H), 0.83 (s, 3H), 0.73-0.67 (m, 1H), 0.65 (s, 3H), 0.64-0.57 (m, 1H), 0.51-0.44 (m, 1H), 0.43-0.35 (m, 1H), 0.17-0.10 (m, 1H), 0.09-0.03 (m, 1H). LC-ELSD/MS purity >99%, MS ESI calcd. for C32H₅₆₀₂ [M−2H₂O+H]⁺ 437.4, found 437.4.

### Example 30: Synthesis of (2S,4aR,4bS,6aR,7S,7aS,8aR,8bS,8cR)-2-ethyl-7-((R)-6-hydroxy-6-methylheptan-2-yl)-4a,6a-dimethyl-1,2,3,4,4a,4b,5,6,6a,7,7a,8,8a,8b,8c,9-hexadecahydrocyclopropa[4,5]cyclopenta[1,2-a]phenanthren-2-ol (B-20)

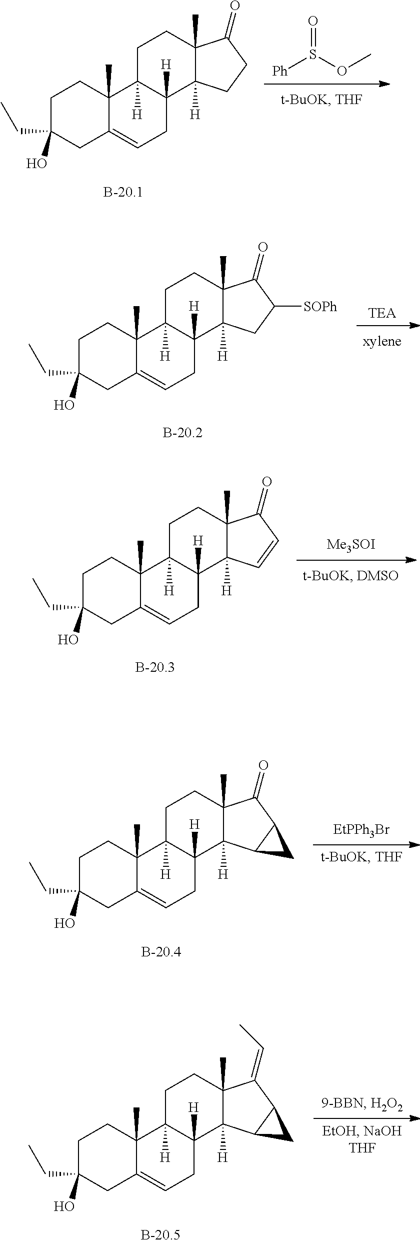

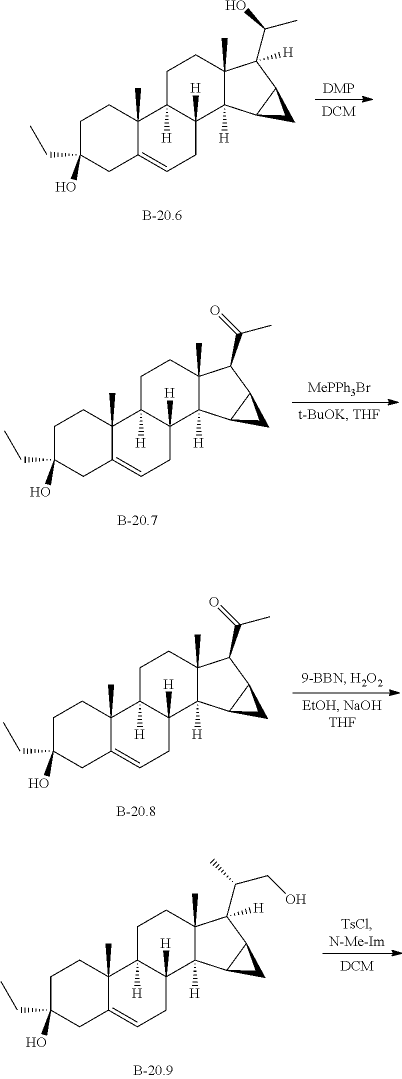

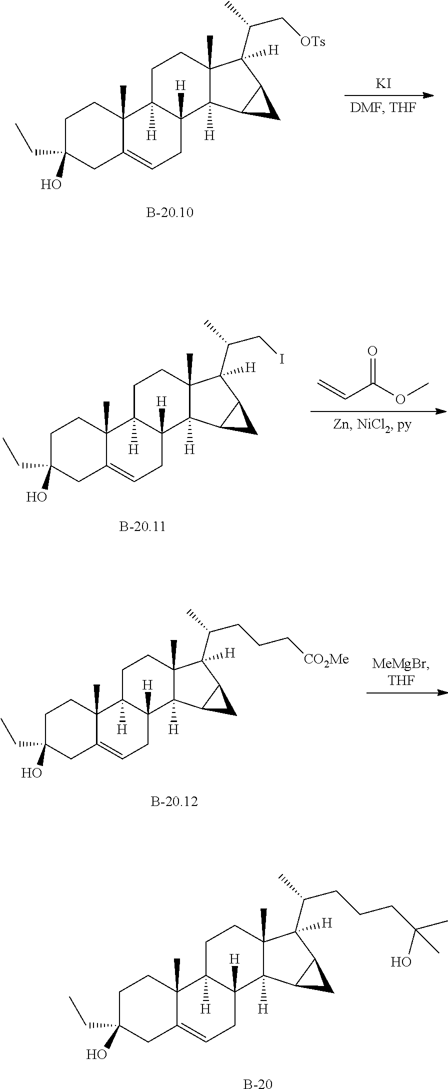

### Synthesis of B-20.2

To a suspension of t-BuOK (7.06 g, 63.0 mmol) in THF (200 mL) was added B-20.1 (10.0 g, 31.5 mmol; preparation described in WO2014/160480A1) at 15° C. under N₂. The mixture was stirred at 15° C. for 10 minutes and then methyl benzenesulfinate (7.97 mL, 63.0 mmol) was added. The mixture was stirred at 30° C. for 0.5 hour. The mixture was quenched with H₂O (220 mL) and extracted with EtOAc (2×500 mL). The organic layer was separated, dried over Na₂SO₄, filtered, and concentrated to give B-20.2 (14.5 g) that was carried on to the next step without further purification.

### Synthesis of B-20.3

To a mixture of B-20.2 (19.0 g, 43.1 mmol) in xylene (400 mL) was added triethylamine (19 ml, 136 mmol) in portions. The reaction mixture was stirred at 140° C. for 16 hours under N₂ and then was filtered and concentrated. The residue was purified by flash column (0-40% of EtOAc in PE/DCM (1/1)) to give the product B-20.3 (6.80 g, 50%). 1H NMR (400 MHz, CDCl₃) δ_{H} 7.55-7.45 (m, 1H), 6.09-6.00 (m, 1H), 5.40-5.30 (m, 1H), 2.45-2.25 (m, 3H), 2.15-2.05 (m, 1H), 1.85-1.50 (m, 8H), 1.45-1.35 (m, 2H), 1.30-1.20 (m, 2H), 1.18-0.95 (m, 8H), 0.92-0.80 (m, 3H).

### Synthesis of B-20.4

A stirred solution of trimethylsulfoxonium iodide (3.91 g, 17.8 mmol) and t-BuOK (1.99 g, 17.8 mmol) in DMSO (20 mL) and THF (10 mL) was heated at 60° C. for 1 hour under N₂. B-20.3 (2.80 g, 8.90 mmol) in DMSO (8 mL) and THF (4 mL) was added to the reaction mixture and stirred at 25° C. for 2 hours. The reaction was treated with water (200 mL) and extracted with EtOAc (3×100 mL). The combined organic phase was washed with water (3×100 mL) and brine (100 mL), dried over anhydrous Na₂SO₄, filtered, and concentrated to afford B-20.4 (3.00 g). ¹H NMR (400 MHz, CDCl₃) δ_{H} 5.38-5.34 (m, 1H), 2.41-2.32 (m, 2H), 1.99-1.93 (m, 2H), 1.83-1.70 (m, 6H), 1.42-1.22 (m, 8H), 1.06 (s, 3H), 0.97 (s, 3H), 0.90-0.81 (m, 7H).

### Synthesis of B-20.5

To a mixture of EtPPh₃Br (25.0 g, 67.5 mmol) in THF (80 mL) was added t-BuOK (4.86 g, 67.5 mmol) at 15° C. under N₂. The resulting mixture was stirred at 50° C. for 30 minutes. B-20.4 (7.40 g, 22.5 mmol) in THF (20 mL) was added in portions below 50° C. and the mixture was stirred at 50° C. for 1 hour. The reaction mixture was quenched with 10% NH₄Cl (500 mL) and extracted with EtOAc (2×800 mL). The combined organic phase was washed with brine (100 mL), dried over anhydrous Na₂SO₄, filtered, and concentrated. The residue was purified by trituration with MeOH/H₂O (1:1, 500 mL) to give B-20.5 (8.22 g). 1H NMR (400 MHz, CDCl₃) δ_{H}·37-5.32 (m, 1H), 2.44-2.32 (m, 1H), 2.25-2.15 (m, 1H), 2.10-2.03 (m, 1H), 1.92-1.84 (m, 1H), 1.78-1.34 (m, 18H), 1.30-1.23 (m, 3H), 1.06 (s, 3H), 0.92-0.82 (m, 6H), 0.68-0.58 (m, 1H)

### Synthesis of B-20.6

To a solution of B-20.5 (8.22 g, 24.1 mmol) in THF (90 mL) was added 9-BBN (192 mL, 96.0 mmol, 0.5 M in THF) at 25° C. under N₂ and the reaction was stirred for 16 hours. To the resulting mixture was added ethanol (13.3 g, 289 mmol) at 25° C., followed by NaOH (57.8 mL, 5.0 M, 289 mmol) at 0° C. H₂O₂ (28.9 mL, 10 M, 289 mmol) was added dropwise at 0° C. The reaction mixture was stirred at 80° C. for 1 hour. The mixture was added to Na₂SO₃ (200 mL, 20% aq.) and extracted with EtOAc (2×800 mL). The combined organic layer was washed with brine (2×400 mL), dried over Na₂SO₄, filtered, and concentrated to give B-20.6 (7.00 g, 81%). ¹H NMR (400 MHz, CDCl₃) δ_{H} 5.35 (d, J=5.2 Hz, 1H), 3.90-3.75 (m, 2H), 2.47-1.83 (m, 8H), 1.54-1.42 (m, 9H), 1.35-1.12 (m, 8H), 1.04 (s, 3H), 0.95-0.8 (m, 4H), 0.80-0.75 (m, 3H)

### Synthesis of B-20.7

To a solution of B-20.6 (6.00 g, 16.7 mmol) in DCM (60 mL) was added DMP (21.2 g, 50.0 mmol) at 30° C. The reaction mixture was stirred at 40° C. for 1 hour. The mixture was quenched with saturated NaHCO₃ (100 mL) and extracted with DCM (3×100 mL). The combined organic phase was washed with NaHCO₃/Na₂S₂O₃ aqueous (1:1, 2×100 mL), brine (50 mL), dried over Na₂SO₄, filtered, and concentrated to give B-20.7 (13.0 g). 1H NMR (400 MHz, CDCl₃) δ_{H} 5.34 (d, J 4.8 Hz, 1H), 2.74 (d, J 4.0 Hz, 1H), 2.55-2.48 (m, 1H), 2.41-2.30 (m, 1H), 2.22 (s, 3H), 2.19-1.87 (m, 11H), 1.32-1.21 (m, 5H), 1.04 (s, 3H), 0.93-0.82 (m, 6H), 0.73 (s, 3H), 0.47-0.36 (m, 1H).

### Synthesis of B-20.8

To a solution of PPh₃MeBr (20.0 g, 56.0 mmol) in THF (30 mL) was added t-BuOK (4.71 g, 42.0 mmol) and the reaction mixture was stirred for 0.5 hour at 50° C. A solution of B-20.7 (5.00 g, 14.0 mmol) in THF (20 mL) was added into the reaction at 50° C. The reaction mixture was stirred for 3 hours at 50° C. The mixture was poured into saturated NH₄Cl (300 mL) and extracted with EtOAc (2×500 mL). The combined organic phase was washed with saturated brine (300 mL), dried over Na₂SO₄, filtered, and concentrated to give B-20.8 (4.00 g, 80%). ¹H NMR (400 MHz, CDCl₃) δ_{H} 5.37-5.32 (m, 1H), 5.11 (s, 1H), 4.90-4.80 (m, 1H), 2.46-2.29 (m, 2H), 2.30-2.15 (m, 2H), 2.10-1.99 (m, 3H), 1.85-1.70 (m, 8H), 1.35-1.20 (m, 5H), 1.10-0.95 (m, 6H), 0.90-0.80 (m, 4H), 0.75-0.68 (m, 1H), 0.66 (s, 3H), 0.32-0.17 (m, 1H).

### Synthesis of B-20.9

To a solution of B-20.8 (2.00 g, 5.64 mmol) in THF (20 mL) was added 9-BBN (45.0 mL, 22.5 mmol, 0.5 M in THF) at 25° C. under N₂ and the reaction was stirred for 16 hours. To the resulting mixture was added ethanol (3.11 g, 67.6 mmol) at 25° C., followed by NaOH (13.5 mL, 5.0 M, 67.6 mmol) at 0° C. H₂O₂ (6.76 mL, 10 M, 67.6 mmol) was added dropwise at 0° C. The reaction mixture was stirred at 80° C. for 1 hour. The mixture was added to Na₂SO₃ (200 mL, 20% aq.) and extracted with EtOAc (2×500 mL). The combined organic layer was washed with brine (2×200 mL), dried over Na₂SO₄, filtered, and concentrated to give B-20.9 (1.10 g, 52%). ¹H NMR (400 MHz, CDCl₃) δ_{H} 5.47-5.32 (m, 1H), 3.88-3.82 (m, 1H), 3.66-3.60 (m, 1H), 2.40-2.34 (m, 1H), 2.22-2.16 (m, 1H), 2.08-2.04 (m, 1H), 1.78-1.60 (m, 6H), 1.48-1.35 (m, 6H), 1.21-1.08 (m, 6H), 1.05 (s, 3H), 1.03-1.00 (m, 3H), 0.95-0.75 (m, 5H), 0.78 (s, 3H), 0.69-0.60 (m, 1H), 0.16-0.08 (m, 1H).

### Synthesis of B-20.10

To a solution of B-20.9 (1.10 g, 2.95 mmol) in DCM (15 mL) was added N-methylimidazole (1.20 g, 14.7 mmol) and TsCl (1.12 g, 5.90 mmol) and the mixture was stirred at 25° C. for 1 hour. H₂O (50 mL) was added and the mixture was washed with 1M HCl (50 mL) and NaHCO₃ (50 mL). The combined organic phase was washed with brine (2×50 mL), dried over anhydrous Na₂SO₄, filtered, and concentrated. The residue was purified by flash column (0˜25% of EtOAc in PE) and further purified by SFC (Column: DAICEL CHIRALPAK AD (250 mm*30 mm, 10 um); Condition: 0.1% NH₃H₂O IPA; Begin B: 45%; End B: 45%; FlowRate (ml/min): 80) to afford B-20.10 (0.50 g, 45%). ¹H NMR (400 MHz, CDCl₃) δ_{H} 7.85-7.75 (m, 2H), 7.34 (d, J=8.0 Hz, 2H), 5.40-5.25 (m, 1H), 2.47-2.33 (m, 6H), 2.25-2.00 (m, 6H), 1.37-1.21 (m, 5H), 1.21-0.98 (m, 11H), 0.97-0.92 (m, 4H), 0.90-0.80 (m, 4H), 0.71 (s, 3H), 0.57-0.52 (m, 1H), 0.09-0.00 (m, 1H).

### Synthesis of B-20.11

To a solution of B-20.10 (0.50 g, 949 umol) in DMF (5 mL) and THF (2 mL) was added KI (755 mg, 4.55 mmol) at 25° C. The mixture was stirred at 60° C. for 5 hours then poured into water (20 mL) and extracted with EtOAc (3×50 mL). The combined organic phase was washed with saturated brine (2×30 mL), dried over anhydrous Na₂SO₄, filtered, and concentrated to give B-20.11 (0.50 g). ¹H NMR (400 MHz, CDCl₃) δ_{H} 5.40-5.30 (m, 1H), 3.69-3.32 (m, 2H), 2.44-2.31 (m, 1H), 2.23-2.12 (m, 1H), 2.10-2.00 (m, 1H), 1.50-1.20 (m, 12H), 1.15-0.95 (m, 12H), 0.94-0.85 (m, 4H), 0.79 (s, 3H), 0.66-0.61 (m, 1H), 0.20-0.08 (m, 1H)

### Synthesis of B-20.12

To a mixture of Zn powder (471 mg, 7.21 mmol) in dry pyridine (10 mL) was added slowly methyl acrylate (620 mg, 7.21 mmol) and NiCl₂ (199 mg, 1.54 mmol) at 25° C. under N₂. After stirring at 60° C. for 2 hours, a solution of B-20.11 (0.50 g, 1.03 mmol) was added. The reaction mixture was stirred at 25° C. for another 3.5 hours. The mixture was poured into aq. NH₄Cl (20 mL) and extracted with EtOAc (2×50 mL). The organic phase was washed with brine (2×30 mL), dried over Na₂SO₄, filtered, and concentrated. The residue was purified by flash column chromatography (0-15% of EtOAc in PE) to give B-20.12 (280 mg, 62%). ¹H NMR (400 MHz, CDCl₃) δ_{H} 5.34 (d, J=4.8 Hz, 1H), 3.67 (s, 3H), 2.42-2.24 (m, 3H), 2.22-2.13 (m, 1H), 2.10-2.02 (m, 1H), 1.84-1.60 (m, 9H), 1.51-1.06 (m, 12H), 1.04 (s, 3H), 1.03-0.88 (m, 5H), 0.87-0.82 (m, 3H), 0.75 (s, 3H), 0.63-0.55 (m, 1H), 0.12-0.00 (m, 1H). LC-ELSD/MS 30-90AB_2 min_E, purity>99%, MS ESI calcd. for C₂₉H₄₆O₃ [M+H−H₂O]⁺ 425.3, found 425.4.

### Synthesis of B-20

A solution of MeMgBr (4.83 mL, 3 M in Et₂O, 14.5 mmol) in THF (2 mL) was reacted with B-20.12 (0.23 g, 519 umol) in THF (2 mL) at 0° C. under N₂. The mixture was stirred at 20° C. for 3 hours and then quenched with NH₄Cl (50 mL) and extracted with EtOAc (3×50 mL). The combined organic phase was washed with brine (20 mL), dried over Na₂SO₄, filtered, and concentrated. The residue was purified to give B-20 (98.3 mg, 90%). 1H NMR (400 MHz, CDCl₃) δ_{H} 5.40-5.29 (m, 1H), 2.47-2.31 (m, 1H), 2.25-2.12 (m, 1H), 2.09-1.97 (m, 1H), 1.83-1.58 (m, 8H), 1.53-1.28 (m, 10H), 1.25-1.20 (m, 9H), 1.20-1.05 (m, 4H), 1.04 (s, 3H), 0.98-0.82 (m, 7H), 0.75 (s, 3H), 0.63-0.54 (m, 1H), 0.11-0.02 (m, 1H). LC-ELSD/MS 30-90AB_2 min_E, purity>99%, MS ESI calcd. for C₃₀H₅₀O₂ [M+H-2H₂O]⁺ 407.4, found 407.4.

### Example 31: Synthesis of (3S,8R,9S,10R,13R,14S,15R,17R)-3-ethyl-17-((R)-6-hydroxy-6-methylheptan-2-yl)-10,13,15-trimethyl-2,3,4,7,8,9,10,11,12,13,14,15,16,17-tetradecahydro-1H-cyclopenta[a]phenanthren-3-ol (B-21)

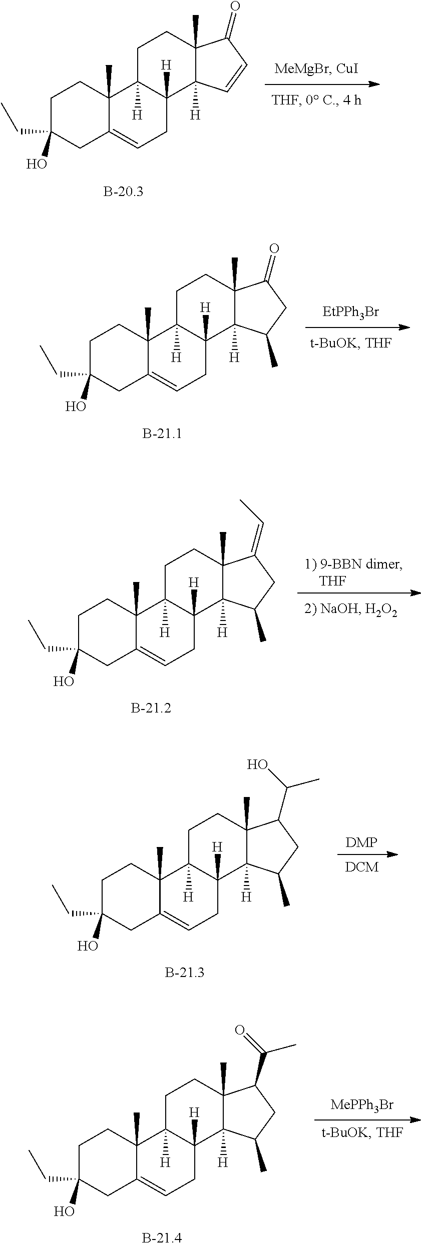

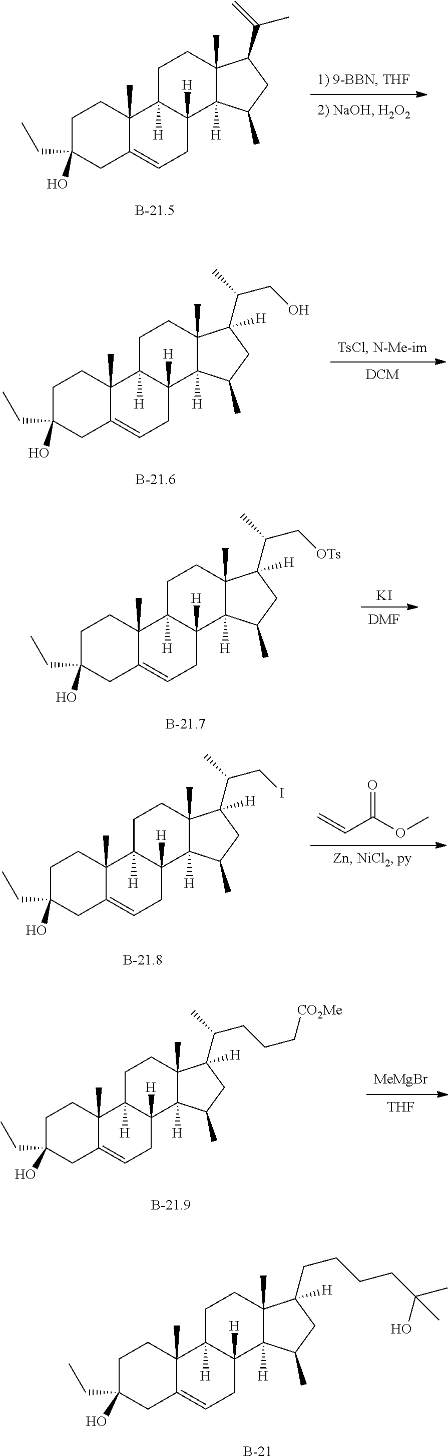

### Synthesis of B-21.1

To a solution of MeMgBr (20.2 mL, 60.8 mmol, 3 M in Et₂O) in THF (70 mL) was added CuI (5.78 g, 30.4 mmol) at 0° C. and the mixture was stirred for 1 hour. The mixture was reacted with B-20.3 (4.80 g, 15.2 mmol) in THF (90 mL) at 0° C. and the mixture was stirred for 2 hours. The mixture was poured into saturated NH₄Cl (120 mL) and extracted with EtOAc (2×60 mL). The combined organic layer was washed with brine (2×40 mL), dried over anhydrous Na₂SO₄, filtered, and concentrated. The residue was purified by flash column chromatography (0%˜25% of EtOAc in PE) to give B-21.1 (3.60 g, 72%). ¹H NMR (400 MHz, CDCl₃) δ_{H} 5.47-5.27 (m, 1H), 2.56-2.05 (m, 6H), 1.90-1.61 (m, 7H), 1.59-1.27 (m, 7H), 1.16-1.07 (m, 6H), 1.04 (s, 3H), 0.90-0.83 (m, 3H)

### Synthesis of B-21.2

To a mixture of EtPPh₃Br (7.20 g, 19.4 mmol) in THF (40 mL) was added t-BuOK (2.17 g, 19.4 mmol) at 15° C. under N₂ and the mixture was stirred at 50° C. for 30 min. Compound B-21.1 (3.60 g, 10.8 mmol) was added in portions below 50° C. The reaction mixture was stirred at 50° C. for 16 hours and then quenched with 10% NH₄Cl aqueous (150 mL) at 15° C. The THF layer was separated and the combined organic phase was washed with brine (2×100 mL), dried over anhydrous Na₂SO₄, filtered, and concentrated. The residue was purified by silica gel chromatography (PE/EtOAc=0%˜20%) to afford B-21.3 (1.70 g, 46%). ¹H NMR (400 MHz, CDCl₃) δ_{H} 5.36-5.27 (m, 1H), 5.19-5.09 (m, 1H), 2.65-2.50 (m, 1H), 2.37 (br d, J=13.2 Hz, 1H), 2.24-2.02 (m, 5H), 1.77-1.71 (m, 2H), 1.59-1.28 (m, 12H), 1.11 (s, 3H), 1.08 (s, 3H), 0.95 (d, J=7.2 Hz, 3H), 0.87 (s, 3H), 0.86 (s, 3H)

### Synthesis of B-21.3

To a solution of B-21.2 (1.7 g, 4.97 mmol) in THF (20 mL) was added 9-BBN dimer (3.61 g, 14.8 mmol) at 25° C. The mixture was stirred at 45° C. for 16 h. To the resulting mixture was added ethanol (2.27 g, 49.5 mmol) at 0° C. followed by NaOH (9.90 mL, 5M, 49.5 mmol). H₂O₂ (5.6 g, 30%, 49.5 mmol) was added dropwise at 0° C. After addition, the mixture was stirred at 80° C. for 1 hour. The mixture was added to saturated Na₂S₂O₃ (70 mL), stirred for 30 mins, and then extracted with EtOAc (100 mL). The combined organic phase was washed with brine (2×100 mL), dried over anhydrous Na₂SO₄, filtered, and concentrated. The residue was purified by flash column chromatography (0˜25% of EtOAc in PE) to give B-21.3 (1.7 g, 95%). ¹H NMR (400 MHz, CDCl₃) δ_{H} 5.30 (br d, J=5.2 Hz, 1H), 3.82-3.66 (m, 1H), 2.44-2.07 (m, 4H), 1.87-1.31 (m, 19H), 1.17-1.09 (m, 3H), 1.06 (s, 3H), 0.95 (d, J=7.2 Hz, 3H), 0.87-0.83 (m, 6H).

### Synthesis of B-21.4

To a solution of B-21.3 (1.20 g, 3.32 mmol) in DCM (20 mL) was added DMP (2.20 g, 5.18 mmol) at 25° C. and the mixture was stirred for 0.5 h. The mixture was added to aq. NaHCO₃ (50 mL, sat.) and aq. Na₂S₂O₃ (50 mL, sat.). The aqueous phase was extracted with DCM (2×50 mL). The combined organic phase was washed with NaHCO₃/Na₂S₂O₃ (1:1, 2×50 mL, sat.), brine (100 mL), dried over anhydrous Na₂SO₄, filtered, and concentrated to give B-21.4 (1.0 g, 83%). ¹H NMR (400 MHz, CDCl₃) δ_{H} 5.30 (d, J=5.2 Hz, 1H), 2.56-2.33 (m, 2H), 2.12 (s, 3H), 2.142.11 (m, 1H), 2.00-1.94 (m, 1H), 1.88-1.58 (m, 8H), 1.51-1.20 (m, 10H), 1.16-1.11 (m, 1H), 1.06 (s, 3H), 0.97 (d, J=7.2 Hz, 3H), 0.86 (t, J=7.6 Hz, 3H), 0.79 (s, 3H)

### Synthesis of B-21.5

To a mixture of MePPh₃Br (8.46 g, 23.7 mmol) in THF (45 mL) was added t-BuOK (2.65 g, 23.7 mmol) at 15° C. under N₂ and the mixture was stirred for 30 min. Compound B-21.4 (1.70 g, 4.74 mmol) was added in portions below 50° C. The reaction mixture was stirred at 50° C. for 4 hours then quenched with 10% NH₄Cl (150 mL) at 15° C. The THF layer was separated and concentrated. The residue was triturated with (MeOH/H₂O=1/1 160 mL) at 20° C., washed with water (200 mL), dissolved with DCM, dried over anhydrous Na₂SO₄, filtered, and concentrated to give B-21.5 (1.4 g, 83%). ¹H NMR (400 MHz, CDCl₃) δ_{H} 5.34-5.27 (m, 1H), 4.84 (s, 1H), 4.72 (s, 1H), 2.37 (br dd, J=2.4, 13.2 Hz, 1H), 2.24-1.96 (m, 5H), 1.77 (s, 3H), 1.73-1.32 (m, 14H), 1.21-1.15 (m, 2H), 1.07 (s, 3H), 0.96 (d, J=7.2 Hz, 3H), 0.86 (t, J=7.2 Hz, 3H), 0.73 (s, 3H).

### Synthesis of B-21.6

To a solution of B-21.5 (1.40 g, 3.92 mmol) in THF (20 mL) was added 9-BBN dimer (5.40 g, 22.1 mmol) at 25° C. The mixture was stirred at 45° C. for 16 h. To the resulting mixture was added ethanol (6.81 g, 117 mmol) at 0° C. Then NaOH aqueous (23.4 mL, 5M, 117 mmol) was added at 0° C. followed by H₂O₂ (11.7 ml, 10 M, 117 mmol) dropwise. After the addition, the mixture was stirred at 80° C. for 1 hour. The mixture was added to saturated Na₂S₂O₃ (70 mL), stirred for 30 mins, and then extracted with EtOAc (100 mL). The combined organic phase was washed with brine (2×100 mL), dried over anhydrous Na₂SO₄, filtered, and concentrated. The residue was purified by flash column chromatography (0˜25% of EtOAc in PE) to give B-21.6 (1 g, 68%). ¹H NMR (400 MHz, CDCl₃) δ_{H} 5.35-5.26 (m, 1H), 3.64 (dd, J=3.6, 10.4 Hz, 1H), 3.40 (dd, J=6.4, 10.4 Hz, 1H), 2.43-1.89 (m, 7H), 1.79-1.59 (m, 8H), 1.49-1.43 (m, 6H), 1.26 (s, 3H), 1.07-1.04 (m, 6H), 0.91 (d, J=7.2 Hz, 3H), 0.86-0.83 (m, 6H)

### Synthesis of B-21.7

To a solution of B-21.6 (1.0 g, 2.66 mmol) in DCM (10 mL) was added N-Me-Im (327 mg, 3.99 mmol), TEA (0.739 ml, 5.32 mmol) and TsCl (1.01 g, 5.32 mmol). The mixture was stirred at 25° C. for 1 h. The reaction was treated with water (50 mL) and extracted with DCM (2×50 mL). The combined organic phase was washed with brine (50 mL), dried over anhydrous Na₂SO₄, filtered, and concentrated. Purification by SFC (DAICEL CHIRALPAK AD (250 mm*30 mm, 10 um)); condition: 0.1% NH₃H₂O IPA; gradient: from 35% to 35% of B, FlowRate (ml/min): 70) provided B-21.7 (470 mg, 47%). 1H NMR (400 MHz, CDCl₃) δ_{H} 7.79 (d, J=8.4 Hz, 2H), 7.34 (d, J=8.0 Hz, 2H), 5.30-5.27 (m, 1H), 3.96 (dd, J=2.8, 9.2 Hz, 1H), 3.81 (dd, J=6.4, 9.2 Hz, 1H), 2.45 (s, 3H), 2.39-2.31 (m, 1H), 2.18-1.98 (m, 4H), 1.94-1.81 (m, 1H), 1.90-1.81 (m, 1H), 1.77-1.09 (m, 17H), 1.04 (s, 3H), 0.99 (d, J=6.8 Hz, 3H), 0.89-0.84 (m, 6H), 0.78 (s, 3H).

### Synthesis of B-21.8

To a solution of B-21.7 (470 mg, 0.889 mmol) in DMF (5 mL) was added KI (737 mg, 4.44 mmol) at 25° C. The mixture was stirred at 60° C. for 1 hour then poured into water (30 mL) and stirred for 20 min. The aqueous phase was extracted with EtOAc (3×50 mL). The combined organic phase was washed with brine (2×20 mL), dried over anhydrous Na₂SO₄, filtered, and concentrated to give B-21.8 (410 mg, 95%). ¹H NMR (400 MHz, CDCl₃) δ_{H} 5.35-5.25 (m, 1H), 3.33 (dd, J=2.4, 9.6 Hz, 1H), 3.18 (dd, J=5.2, 9.6 Hz, 1H), 2.42-2.07 (m, 4H), 1.95-1.85 (m, 1H), 1.78-1.32 (m, 12H), 1.23-1.08 (m, 5H), 1.06 (s, 3H), 1.03 (d, J=6.0 Hz, 3H), 0.99-0.95 (m, 1H), 0.92 (d, J=7.6 Hz, 3H), 0.88-0.82 (m, 6H).

### Synthesis of B-21.9

To a mixture of Zn powder (188 mg, 2.88 mmol) in dry pyridine (4 mL) was added slowly methyl acrylate (247 mg, 2.88 mmol) and NiCl₂ (80.2 mg, 618 umol) at 25° C. under N₂. After stirring for 2 hours, a solution of B-21.8 (200 mg, 412 umol) in pyridine (2 mL) was added to the mixture slowly. The reaction mixture was stirred for 20 hrs at 25° C. then diluted with NH₄Cl (30 mL). The mixture was filtered through a pad of silica gel. The filtrate was extracted with EtOAc (2×20 mL) and washed with HCl (4×50 mL, 0.5M). The organic phase was dried over Na₂SO₄, filtered, and concentrated to give B-21.9 (200 mg). ¹H NMR (400 MHz, CDCl₃) δ_{H} 5.38-5.20 (m, 1H), 3.67-3.66 (m, 1H), 3.67 (s, 2H), 2.44-2.10 (m, 6H), 1.97-1.88 (m, 2H), 1.79-1.56 (m, 8H), 1.53-1.32 (m, 7H), 1.22-1.09 (m, 3H), 1.06 (s, 3H), 1.03-0.97 (m, 1H), 1.03-0.97 (m, 1H), 0.94 (br d, J=6.8 Hz, 2H), 0.95-0.92 (m, 1H), 0.90 (d, J=7.2 Hz, 3H), 0.87-0.84 (m, 3H), 0.84-0.81 (m, 3H).

### Synthesis of B-21

A solution of MeMgBr (2.39 mL, 7.19 mmol, 3M in Et₂O) in THF (8 mL) was reacted with B-21.9 (160 mg, 0.359 mmol) in THF (2 mL) at 0° C. under N₂. The mixture was stirred at 0° C. for 10 min and then concentrated at 25° C. The mixture was poured into aq. NH₄Cl (20 mL) and stirred for 20 min. The aqueous phase was extracted with EtOAc (3×40 mL). The combined organic phase was dried over anhydrous Na₂SO₄, filtered, and concentrated. The residue was purified by flash column chromatography (0˜20% of EtOAc in PE) to give B-21 (44.1 mg, 28%). ¹H NMR (400 MHz, CDCl₃) δ_{H} 5.30 (br d, J=5.6 Hz, 1H), 2.36 (br d, J=10.8 Hz, 1H), 2.29-2.10 (m, 2H), 2.04 (br dd, J=2, 13.2 Hz, 3H), 1.80-1.55 (m, 8H), 1.54-1.29 (m, 16H), 1.22 (s, 6H), 1.06 (s, 3H), 0.92 (dd, J=6.8, 13.2 Hz, 6H), 0.88-0.81 (m, 6H). LC-ELSD/MS 30-90AB_2 min_E, purity>98%, MS ESI calcd. for C₃₀H₅₂O₂ [M−2H₂O+H]⁺ 409, found 409.

### Example 32: Synthesis of (3S,5S,8R,9S,10S,13S,14S,16R,17S)-3-ethyl-17-((R)-6-hydroxy-6-methylheptan-2-yl)-10,13,16-trimethylhexadecahydro-1H-cyclopenta[a]phenanthren-3-ol (B-22)

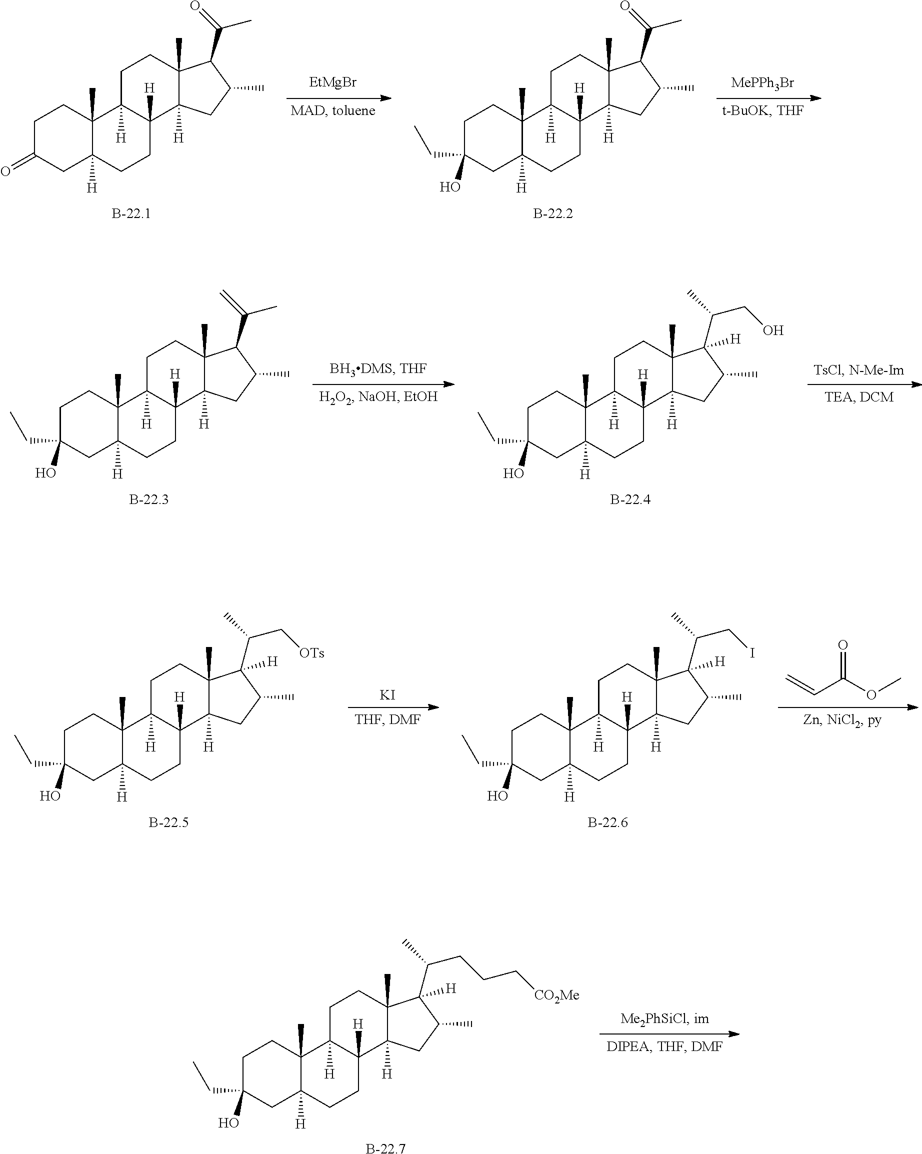

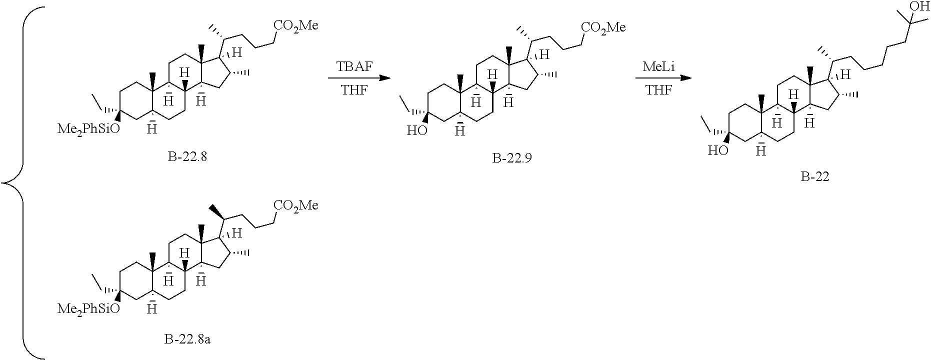

### Synthesis of B-22.2

To a solution of BHT (99.6 g, 452 mmol) in toluene (100 mL) under nitrogen at 0° C. was added AlMes (2 M in toluene, 113 mL, 226 mmol) dropwise. The mixture was stirred at 25° C. for 1 hour. To the mixture was added a solution of B-22.1 (25.0 g, 75.6 mmol; preparation described in WO2018/013615A1) in DCM (250 mL) dropwise at −70° C. After stirring at −70° C. for 1 hour under N₂, EtMgBr (50.3 mL, 151 mmol, 3M in ethyl ether) was added drop wise at −70° C. The resulting solution was stirred at −70° C. for another 4 hours. The reaction mixture was poured into saturated aqueous citric acid (850 mL, 10%) at below 10° C. and extracted with EtOAc (2×60 mL). The combined organic layer was dried over Na₂SO₄, filtered, purified by flash column chromatography (0-100% of EtOAc in PE) to give B-22.2 (24.6 g, 20%). A batch of B-22.2 (200.0 mg) was further purified by flash column chromatography (0-20% of EtOAc in PE) and lyophilized to give B-22.2 (24.1 mg). ¹H NMR (400 MHz, CDCl₃) δ_{H} 2.71-2.55 (m, 1H), 2.20-2.05 (m, 4H), 1.96-1.88 (m, 1H), 1.68-1.59 (m, 4H), 1.53-1.19 (m, 13H), 1.14-0.97 (m, 2H), 0.97-0.84 (m, 8H), 0.82 (s, 3H), 0.75-0.82 (m, 1H), 0.62 (s, 3H). LC-ELSD/MS 30-90AB_2 min_E, purity>99%, MS ESI calcd. for C₂₄H₄₀O₂ [M−H₂O+H]⁺ 343.3, found 343.3.

### Synthesis of B-22.3

To a mixture of MePPh₃Br (72.5 g, 203 mmol) in THF (200 mL) was added t-BuOK (22.7 g, 203 mmol) at 55° C. under N₂. The resulting mixture was stirred at 55° C. for 30 minutes. B-22.2 (24.6 g, 67.9 mmol) was added in portions below 55° C. The reaction mixture was stirred at 55° C. for 16 hours and then quenched with 10% NH₄Cl (200 mL) at 15° C. The aqueous was extracted with EtOAc (3×50 mL). The combined organic phase was washed with brine (2×30 mL), dried over Na₂SO₄, filtered, and concentrated. The residue was triturated with MeOH (560 mL) to give B-22.3 (24.8 g). ¹H NMR (400 MHz, CDCl₃) δ_{H} 4.86 (s, 1H), 4.68 (s, 1H), 2.24-2.12 (m, 1H), 2.08-1.79 (m, 1H), 1.80-1.70 (m, 4H), 1.68-0.99 (m, 18H), 0.98-0.92 (m, 4H), 0.91-0.85 (m, 4H), 0.82 (s, 3H), 0.78-0.62 (m, 2H), 0.60 (s, 3H)

### Synthesis of B-22.4

To a solution of B-22.3 (24.0 g, 66.9 mmol) in THF (200 mL) was added BH₃·Me₂S (13.3 mL, 133 mmol) at 25° C. and the mixture was stirred for 16 hours. To the resulting mixture was added ethanol (23.3 mL, 401 mmol) at 25° C. Then NaOH (80.2 mL, 5 M, 401 mmol) was added at 25° C. followed by H₂O₂ (40.1 mL, 10 M, 401 mmol) dropwise. The mixture was stirred at 25° C. for 1 hour then added to Na₂SO₃ (200 mL, 25%) and stirred for 2 hours. The mixture was extracted with EtOAc (3×300 mL). The combined organic phase was washed with brine (2×200 mL), dried over anhydrous Na₂SO₄, filtered, and concentrated. The combined organic phase was concentrated to give B-22.4 (29.0 g), which was carried into the next step without further purification.

### Synthesis of B-22.5

To a solution of B-22.4 (28.0 g, 74.3 mmol) in DCM (280 mL) was added N-methylimidazole (9.11 g, 111 mmol), TEA (30.7 mL, 222 mmol), and TsCl (28.2 g, 148 mmol). The mixture was stirred at 20° C. for 1 hour. HCl (100 mL, 1M) was added to adjust to pH<3. The aqueous phase was extracted with DCM (2×100 mL). The combined organic phase was washed with aq. NaHCO₃ (100 mL) and brine (50 mL), dried over anhydrous Na₂SO₄, filtered, concentrated. Purification by flash column chromatography (15%-25% of EtOAc in PE) provided B-22.5 (18.3 g, 44%).

### Synthesis of B-22.6

To a solution of B-22.5 (18.3 g, 34.2 mmol) in DMF (180 mL) and THF (20 mL) was added KI (27.2 g, 164 mmol) at 25° C. The mixture was stirred at 60° C. for 3 hours then poured into water (200 mL) and stirred for 20 minutes. The aqueous phase was extracted with EtOAc (3×100 mL). The combined organic phase was washed with brine (2×80 mL), dried over anhydrous Na₂SO₄, filtered, and concentrated. The residue was dissolved in EtOAc, washed with 4% LiCl aq. (2×250 mL), and concentrated to give B-22.6 (16.6 g).

### Synthesis of B-22.7

To a mixture of Zn powder (934.00 mg, 14.3 mmol) in dry pyridine (20 mL) was added slowly methyl acrylate (1.23 g, 14.3 mmol) and NiCl₂ (397 mg, 3.07 mmol) at 25° C. under N₂. After stirring at 60° C. for 2 hours, the mixture was cooled to 25° C. and a solution of B-22.6 (1.00 g, 2.05 mmol) was added slowly. The reaction mixture was stirred at 25° C. for another 2 hours. The mixture was diluted with NH₄Cl (40 mL) and filtered through a pad of silica gel. The filtrate was extracted with EtOAc (2×30 mL). The organic phase was dried over Na₂SO₄, filtered, and concentrated to give B-22.7 (918 mg).

### Synthesis of B-22.8 & B-22.8a

To a solution of B-22.7 (918 mg, 2.12 mmol) in THF/DMF (10 ml, 1:1) was added imidazole (2.30 g, 33.9 mmol), DIPEA (4.37 g, 33.9 mmol), and chlorodimethylphenylsilane (2.88 g, 16.9 mmol). The reaction mixture was stirred at 25° C. for 24 hours then poured into aq. NaHCO₃ (30 mL) and extracted with DCM (3×20 mL). The combined organic phase was washed with brine (20 mL), dried over anhydrous Na₂SO₄, filtered, and concentrated. The residue was purified by flash column chromatography (0˜5% of EA in PE) and the diastereomers were separated by SFC (column: DAICEL CHIRALPAK IG (250 mm*30 mm, 10 um); Mobile phase: A: CO₂ B: 0.1% NH₃H₂O ETOH; gradient: from 15% to 15% of B, Flow Rate (mL/min): 60) to give B-22.8 (360 mg, 42%) and B-22.8a (200 mg, 23%).

### Synthesis of B-22.9

To a solution of B-22.8 (300 mg, 0.516 mmol) in THF (5 mL) was added TBAF (4.12 mL, 4.12 mmol, 1M in THF) and the reaction mixture was stirred at 20° C. for 16 hours. The mixture was quenched with saturated NH₄Cl solution (30 mL) and extracted with EtOAc (3×30 mL). The combined organic phase was washed with brine (2×20 mL), dried over anhydrous Na₂SO₄, filtered, and concentrated. The residue was purified by flash column chromatography (0-20% of EtOAc in PE) to give B-22.9 (158 mg, 68%). ¹H NMR (400 MHz, CDCl₃) δ_{H} 3.66 (s, 3H), 2.36-2.22 (m, 2H), 1.94-1.84 (m, 1H), 1.82-1.57 (m, 6H), 1.53-1.04 (m, 19H), 1.03-0.94 (m, 4H), 0.93-0.84 (m, 7H), 0.82 (s, 3H), 0.76-0.62 (m, 5H). LC-ELSD/MS 30-90AB_2 min_E, purity>98%, MS ESI calcd. for C₂₉H₅₀O₃ [M−H₂O+H]⁺ 429.4, found 429.4.

### Synthesis of B-22

To a solution of B-22.9 (136 mg, 0.304 mmol) in THF (1 mL) was added MeLi (1.51 mL, 1 M, 1.51 mmol) at 25° C. and the mixture was stirred at 25° C. for 1 hour. The mixture was poured into saturated NH₄Cl (10 mL) and the aqueous layer was extracted with DCM (3×20 mL). The combined organic layer was washed with brine (2×10 mL), dried over anhydrous Na₂SO₄, filtered, and concentrated. The residue was purified by flash column chromatography (0˜25% of EtOAc in PE) and purified from MeCN/H₂O (10 mL) to give B-22 (1.90 mg, 8%). ¹H NMR (400 MHz, CDCl₃) δ_{H} 1.96-1.70 (m, 2H), 1.46-1.31 (m, 12H), 1.26-1.20 (m 14H), 1.17-1.02 (m, 8H), 0.99 (d, J=6.8 Hz, 3H), 0.93-0.85 (m, 7H), 0.82 (s, 3H), 0.78-0.70 (m, 2H), 0.68 (s, 3H). LC-ELSD/MS 30-90AB_2 min_E, purity>99%, MS ESI calcd. for C₃₀H₅₄O₂ [M−2H₂O+H]⁺ 411.4, found 411.4.

### Example 33: Synthesis of (3S,5S,8R,9S,10S,13S,14S,16R,17S)-3-ethyl-17-((2R,5S)-5-hydroxy-6-methylheptan-2-yl)-10,13,16-trimethylhexadecahydro-1H-cyclopenta[a]phenanthren-3-ol (B-23) & (3S,5S,8R,9S,10S,13S,14S,16R,17S)-3-ethyl-17-((2R,5R)-5-hydroxy-6-methylheptan-2-yl)-10,13,16-trimethylhexadecahydro-1H-cyclopenta[a]phenanthren-3-ol (B-24)

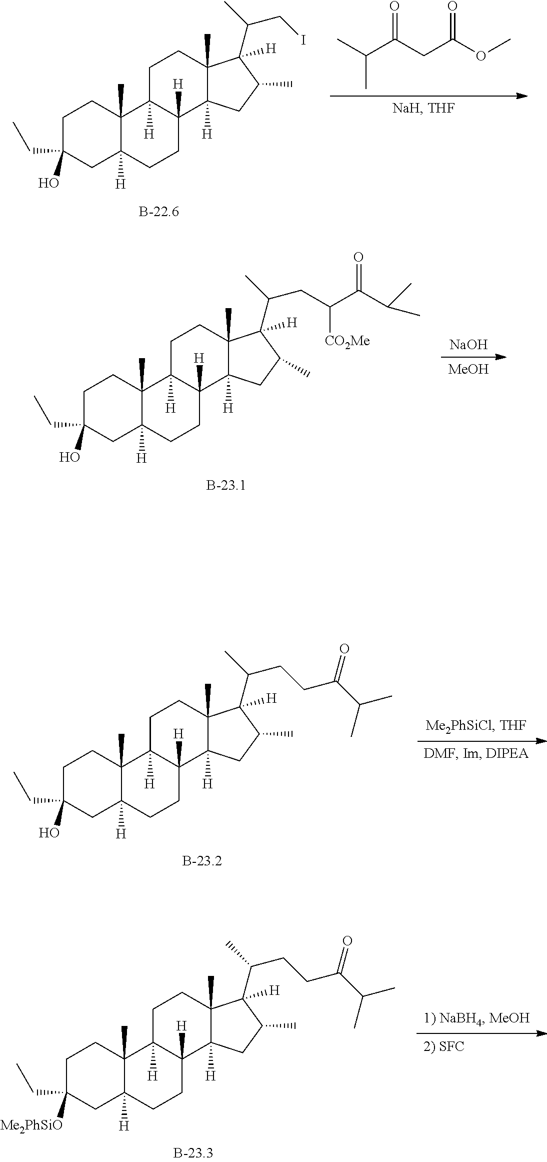

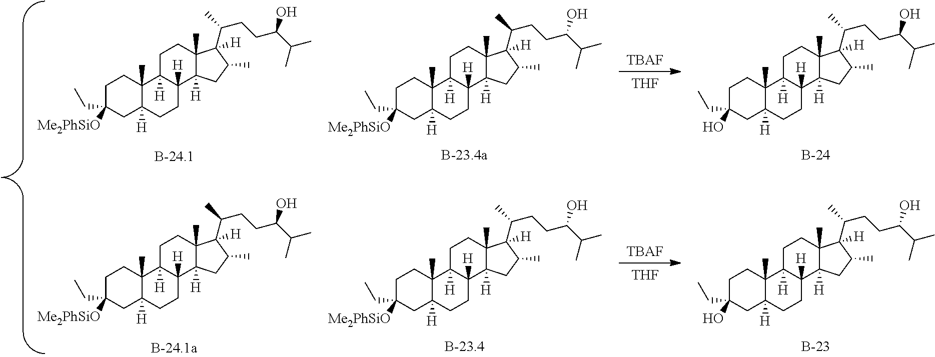

### Synthesis of B-23.1

To a solution of NaH (2.28 g, 57.5 mmol, 60% in mineral oil) in dioxane (90 mL) was added ethyl 3-oxobutanoate (10.6 g, 73.9 mmol) at 0° C. under N₂ and the mixture was stirred for 1 hour. To the mixture was added DMF (100 mL) followed by a solution of B-22.6 (4.00 g, 8.22 mmol) in dioxane (20 mL) dropwise. The reaction mixture was stirred at 100° C. for 16 hours. The mixture was poured into NH₄Cl (200 mL) and the aqueous phase was extracted with DCM (2×300 mL). The combined organic phase was washed with water (2×200 mL), dried over anhydrous Na₂SO₄, filtered, and concentrated to give B-23.1 (16.8 g).

### Synthesis of B-23.2

To a mixture of B-23.1 (12.6 g, 25.0 mmol) in MeOH (30 mL) was added H₂O (10 mL) and NaOH (9.99 g, 250 mmol) at 25° C. The reaction mixture was stirred at 60° C. for 16 hours and then concentrated. The mixture was added to H₂O (50 mL) and extracted with EtOAc (3×50 mL). The combined organic phase was washed with brine (30 mL), dried over anhydrous Na₂SO₄, filtered, and purified by flash column chromatography (0˜20% of EtOAc in PE) to give B-23.2 (3.85 g, 72%). ¹H NMR (400 MHz, CDCl₃) δ_{H} 2.67-2.28 (m, 4H), 1.92-1.78 (m, 3H), 1.45-1.18 (m, 13H), 1.12-1.04 (m, 11H), 1.02-0.98 (m, 3H), 0.91-0.83 (m, 9H), 0.81 (s, 3H), 0.80-0.68 (m, 2H), 0.68-0.65 (m, 3H), 0.65-0.57 (m, 1H).

### Synthesis of B-23.3

To a solution of B-23.2 (4.61 g, 10.3 mmol) in THF/DMF (44 ml, 10:1) was added 1H-imidazole (11.1 g, 164 mmol), DIPEA (21.1 g, 164 mmol), and chlorodimethylphenylsilane (14.0 g, 82.4 mmol). The reaction mixture was stirred at 25° C. for 24 hours, poured into water (50 mL), and extracted with DCM (3×50 mL). The combined organic phase was washed with brine (20 mL), dried over anhydrous Na₂SO₄, filtered, and concentrated. The residue was purified by flash column chromatography (0˜15% of EtOAc in PE) to give B-23.3 (7.30 g). ¹H NMR (400 MHz, CDCl₃) δ_{H} 7.64-7.52 (m, 2H), 7.41-7.32 (m, 3H), 2.67-2.31 (m, 3H), 1.92-1.18 (m, 21H), 1.11-1.07 (m, 8H), 1.02-0.98 (m, 3H), 0.93-0.83 (m, 9H), 0.71-0.63 (m, 7H), 0.40-0.32 (m, 6H).

### Synthesis of B-23.4 & B-23.4a & B-24.1 & B-24.1a

To a solution of B-23.3 (6.30 g, 10.8 mmol) in THF (30 mL) and MeOH (30 ml) was added NaBH₄ (410 mg, 10.8 mmol) at 25° C. The mixture was stirred at 25° C. for 10 minutes then poured into H₂O (100 mL). The aqueous layer was extracted with DCM (3×100 mL). The combined organic layer was washed with brine (2×50 mL), dried over anhydrous Na₂SO₄, filtered, and concentrated. The residue was purified by flash column chromatography (0˜15% of EtOAc in PE) to give the product (3.00 g). The mixture of products (3.00 g) was separated by SFC (column: DAICEL CHIRALPAK AD (250 mm*50 mm, 10 um); Mobile phase: A: CO₂ B: 0.1% NH₃H₂O MEOH; gradient: from 35% to 35% of B, Flow Rate (ml/min): 200) to give a mixture of B-24.1 & B-23.4a & B-23.4 (2.18 g) and B-24.1a (650 mg). B-24.1a (650 mg) was further purified by SFC (column: DAICEL CHIRALPAK AD (250 mm*30 mm, 10 um)); Mobile phase: A: CO₂ B: 0.1% NH₃H₂O MEOH; gradient: from 30% to 30% of B, Flow Rate (ml/min): 70) to give B-24.1a (0.43 g). The mixture of B-24.1 & B-23.4a & B-23.4 (2.18 g) was further purified by SFC (column: DAICEL CHIRALPAK AD (250 mm*50 mm, 10 um); Mobile phase: A: CO₂ B: 0.1% NH₃H₂O ETOH; gradient: from 20% to 20% of B, Flow Rate (ml/min): 200) to give B-24.1 (0.73 g) and mixture of B-23.4a & B-23.4 (1.13 g). The mixture of B-23.4a & B-23.4 (1.13 g) was purified by 1^{st} run SFC (column: DAICEL CHIRALPAK AD (250 mm*50 mm, 10 um); Mobile phase: A: CO₂ B: 0.1% NH₃H₂O IPA; gradient: from 30% to 30% of B, Flow Rate (ml/min): 200) and 2^{nd} run SFC (column: DAICEL CHIRALPAK IG (250 mm*30 mm, 10 um)); Mobile phase: A: CO₂ B: 0.1% NH₃H₂O IPA; gradient: from 25% to 25% of B, Flow Rate (ml/min): 60) to give B-23.4a (0.43 g, 54.0%) & B-23.4 (0.32 g, 40.2%) as colorless oil.

B-24.1: ¹H NMR (400 MHz, CDCl₃) δ_{H} 7.65-7.56 (m, 2H), 7.41-7.30 (m, 3H), 3.43-3.26 (m, 1H), 1.93-1.85 (m, 1H), 1.84-1.74 (m, 1H), 1.70-1.05 (m, 27H), 1.02-0.99 (m, 3H), 0.94-0.82 (m, 14H), 0.70-0.65 (m, 6H), 0.40-0.35 (m, 6H).

B-24.1a: ¹H NMR (400 MHz, CDCl₃) δ_{H} 7.66-7.57 (m, 2H), 7.39-7.29 (m, 3H), 3.36-3.25 (m, 1H), 2.00-1.74 (m, 2H), 1.70-1.07 (m, 25H), 0.99 (d, J=6.8 Hz, 3H), 0.94-0.82 (m, 14H), 0.81-0.76 (m, 1H), 0.73-0.55 (m, 7H), 0.40-0.35 (m, 6H).

B-23.4a: ¹H NMR (400 MHz, CDCl₃) δ_{H} 7.69-7.57 (m, 2H), 7.42-7.29 (m, 3H), 3.37-3.27 (m, 1H), 1.94-1.44 (m, 14H), 1.39-1.23 (m, 6H), 1.18-1.03 (m, 7H), 0.99 (d, J=6.8 Hz, 3H), 0.96-0.80 (m, 15H), 0.79-0.74 (m, 1H), 0.70-0.64 (m, 6H), 0.40-0.35 (m, 6H).

B-23.4: ¹H NMR (400 MHz, CDCl₃) δ_{H} 7.64-7.57 (m, 2H), 7.37-7.31 (m, 3H), 3.37-3.29 (m, 1H), 1.93-1.12 (m, 25H), 1.05-0.95 (m, 4H), 0.94-0.82 (m, 14H), 0.82-0.50 (m, 9H), 0.40-0.35 (m, 6H).

### Synthesis of B-23

To a solution of B-23.4 (0.32 g, 0.5 mmol) in THF (2 mL) was added TBAF (1.09 mL, 1.09 mmol, 1M in THF) and the mixture was stirred at 20° C. for 16 hours. The mixture was quenched with saturated NH₄Cl solution (30 mL) and extracted with EtOAc (3×30 mL). The combined organic phase was washed with brine (2×20 mL), dried over anhydrous Na₂SO₄, filtered, and concentrated. Purification by flash column chromatography (0-30% of EtOAc in PE) followed by trituration with MeCN provided B-23 (135.6 mg, 62%). ¹H NMR (400 MHz, CDCl₃) δ_{H} 3.37-3.29 (m, 1H), 1.94-1.78 (m, 2H), 1.64-1.18 (m, 21H), 1.05-0.96 (m, 4H), 0.96-0.84 (m, 18H), 0.82 (s, 3H), 0.77-0.72 (m, 1H), 0.70-0.63 (m, 4H). LC-ELSD/MS purity>99%, MS ESI calcd. for C₃₀H₅₄O₂ [M−2H₂O+H]⁺ 411.4, found 411.4.

### Synthesis of B-24

To a solution of B-24.1 (0.73 g, 1.25 mmol) was added TBAF (2.5 mL, 2.50 mmol, 1M in THF) and the mixture was stirred at 20° C. for 16 hours. The mixture was quenched with saturated NH₄Cl solution (30 mL) and extracted with EtOAc (3×30 mL). The combined organic phase was washed with brine (2×20 mL), dried over anhydrous Na₂SO₄, filtered, and concentrated. Purification by flash column chromatography (0-30% of EtOAc in PE) followed by lyophilization provided B-24 (291.3 mg, 88%). ¹H NMR (400 MHz, CDCl₃) δ_{H} 3.39-3.30 (m, 1H), 1.94-1.75 (m, 2H), 1.71-1.56 (m, 6H), 1.52-1.05 (m, 21H), 1.02 (d, J=6.8 Hz, 3H), 0.97-0.84 (m, 14H), 0.82 (s, 3H), 0.77-0.72 (m, 1H), 0.68 (s, 3H). LC-ELSD/MS purity>99%, MS ESI calcd. for C₃₀H₅₄O₂ [M−2H₂O+H]⁺ 411.4, found 411.4.

### Example 34: Synthesis of (3R,6R)-6-((3S,5S,8R,9S,10S,13R,14S,17R)-3-ethyl-3-hydroxy-10,13-dimethylhexadecahydro-1H-cyclopenta[a]phenanthren-17-yl)-3-hydroxy-2,2-dimethylheptanenitrile (C-1) & (3S,6R)-6-((3S,5S,8R,9S,10S,13R,14S,17R)-3-ethyl-3-hydroxy-10,13-dimethylhexadecahydro-1H-cyclopenta[a]phenanthren-17-yl)-3-hydroxy-2,2-dimethylheptanenitrile (C-2)

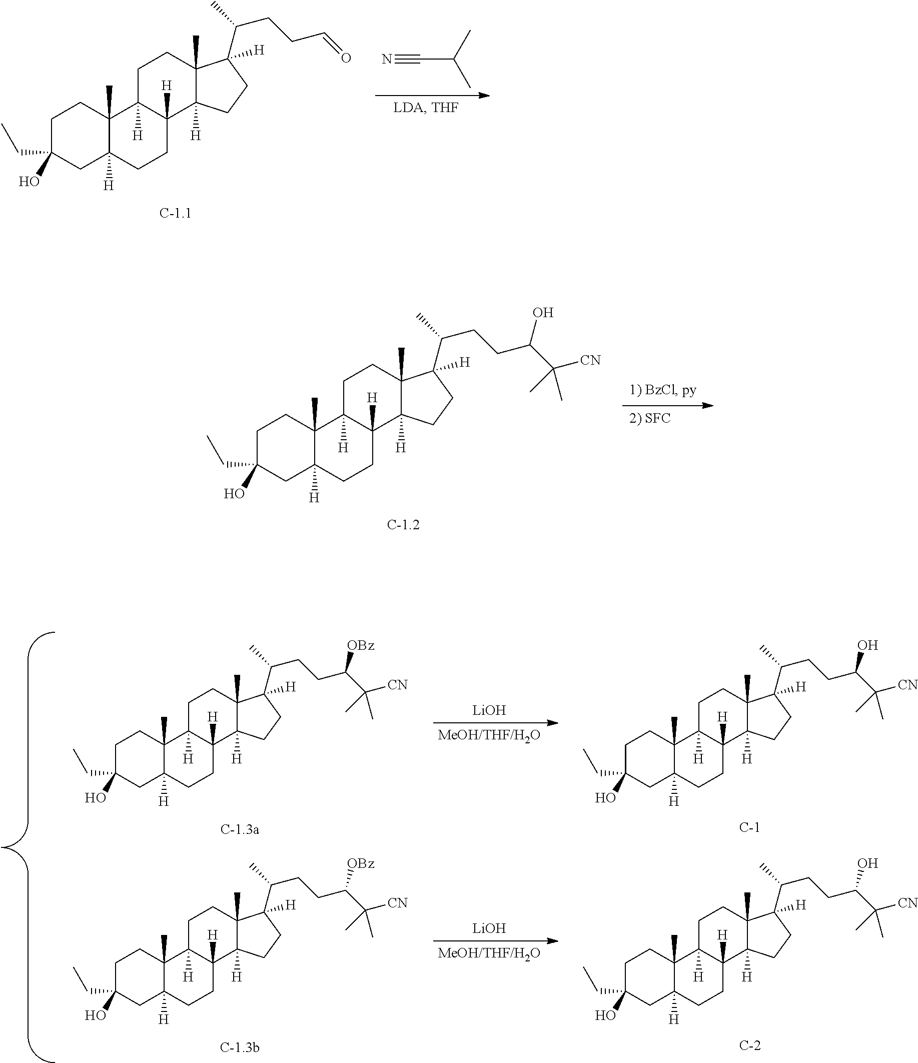

### Synthesis of C-1.2

To a solution of LDA (5.55 mL, 2 M, 11.1 mmol) in THF (20 mL) at −70° C. under N₂, a suspension of isobutyronitrile (1.02 g, 14.8 mmol) was added dropwise and the mixture was stirred for 30 minutes at −70° C. A solution of (R)-4-((3S,5S,8R,9S,10S,13R,14S,17R)-3-ethyl-3-hydroxy-10,13-dimethylhexadecahydro-1H-cyclopenta[a]phenanthren-17-yl)pentanal, C-1.1 (1.5 g, 3.72 mmol) was added the dropwise at −70° C. The mixture was stirred for another 30 mins and then warmed to 25° C. gradually. After stirring at 25° C. for 4 hours, the reaction mixture was quenched with NH₄Cl (40 mL) and extracted with EtOAc (3×80 mL). The combined organic phase was washed with brine (60 mL), dried over Na₂SO₄, filtered and concentrated. Purification by combi-flash (0-15% EtOAc in PE) provided C-1.2 (800 mg, 45%). ¹H NMR (400 MHz, CDCl₃) δ_{H} 3.42-3.40 (m, 1H), 1.99-1.96 (m, 1H), 1.80-1.75 (m, 2H), 1.72-1.59 (m, 5H), 1.44-1.34 (m, 8H), 1.33-1.19 (m, 5H), 1.18-1.08 (m, 3H), 1.08-0.83 (m, 10H), 0.68-0.67 (m, 3H).

### Synthesis of C-1.3a & C-1.3b

To a solution of C-1.2 (800 mg, 1.69 mmol) in pyridine (20 mL) was added benzoyl chloride (355 mg, 2.53 mmol). After stirring at 25° C. for 16 hours, the mixture was added to saturated NH₄Cl (10 mL) and extracted with EtOAc (3×10 mL). The combined organic layer was washed with brine (20 mL), dried over Na₂SO₄, filtered, and concentrated. Purification by combi-flash (0-30% of EtOAc in PE) provided the product, which was further separated by SFC (column: DAICEL CHIRALPAK AD-H (250 mm*30 mm, 5 um)), gradient: 35-35% B (A=0.1% NH₃/H₂O, B=EtOH), flow rate: 60 mL/min) to give C-1.3a (250 mg, Rt=1.267) and C-1.3b (250 mg, Rt=1.515). The configuration of C-24 was randomly assigned.

### Synthesis of C-1

To a solution of C-1.3a (250 mg, 0.444 mmol) in THF (5.7 mL) and MeOH (2.8 mL) was added water (1.4 mL) and LiOH·H₂O (1.91 g, 50.0 mmol). After stirring at 25° cfor 2 hours, water was added (10 mL) and the mixture was extracted with EtOAc (20 mL). The organic layer was separated, dried over Na₂SO₄, filtered, and concentrated. Purification by combi-flash (0-15% of EtOAc in PE) provided C-1 (131.3 mg, 64%). ¹H NMR (400 MHz, CDCl₃) δ_{H} 3.42-3.38 (m, 1H), 1.98-1.95 (m, 1H), 1.90-1.80 (m, 1H), 1.69-1.55 (m, 8H), 1.51-1.41 (m, 4H), 1.41-1.36 (m, 5H), 1.35-1.28 (m, 5H), 1.27-1.16 (m, 5H), 1.16-0.96 (m, 6H), 0.95-0.85 (m, 7H), 0.83 (s, 3H), 0.70-0.60 (m, 4H). LC-ELSD/MS purity>99%, MS ESI calcd. For C₃₀H₅₁NO₂ [M−H₂O+H]⁺ 440.4, found 440.4.

### Synthesis of C-2

To a solution of C-1.3b (250 mg, 0.444 mmol) in THF (5.7 mL) and MeOH (2.8 mL) was added water (1.4 mL) and LiOH·H₂O (1.91 g, 50 mmol). After stirring at 25° C. for 2 hours, the mixture was added to water (10 mL) and extracted with EtOAc (20 mL). The organic layer was separated, dried over Na₂SO₄, filtered, and concentrated. Purification by combi-flash (0-15% of EtOAc in PE) provided C-2 (114.6 mg, 56%). ¹H NMR (400 MHz, CDCl₃) δ_{H} 3.38-3.33 (m, 1H), 1.97-1.94 (m, 1H), 1.83-1.63 (m, 5H), 1.62-1.55 (m, 5H), 1.53-1.42 (m, 3H), 1.41-1.34 (m, 8H), 1.33-1.15 (m, 7H), 1.15-0.97 (m, 7H), 0.95-093 (m, 3H), 0.90-0.86 (m, 4H), 0.82 (s, 3H), 0.70-0.60 (m, 4H). LC-ELSD/MS purity>99%, MS ESI calcd. For C₃₀H₅₁NO₂ [M−H₂O+H]⁺ 440.4 found 440.4.

### Example 35: Synthesis of 1-((1R,4R)-4-((3S,5S,8R,9S,10S,13R,14S,17R)-3-ethyl-3-hydroxy-10,13-dimethylhexadecahydro-1H-cyclopenta[a]phenanthren-17-yl)-1-hydroxypentyl)cyclopropanecarbonitrile (C-3) and 1-((1S,4R)-4-((3S,5S,8R,9S,10S,13R,14S,17R)-3-ethyl-3-hydroxy-10,13-dimethylhexadecahydro-1H-cyclopenta[a]phenanthren-17-yl)-1-hydroxypentyl)cyclopropanecarbonitrile (C-4)

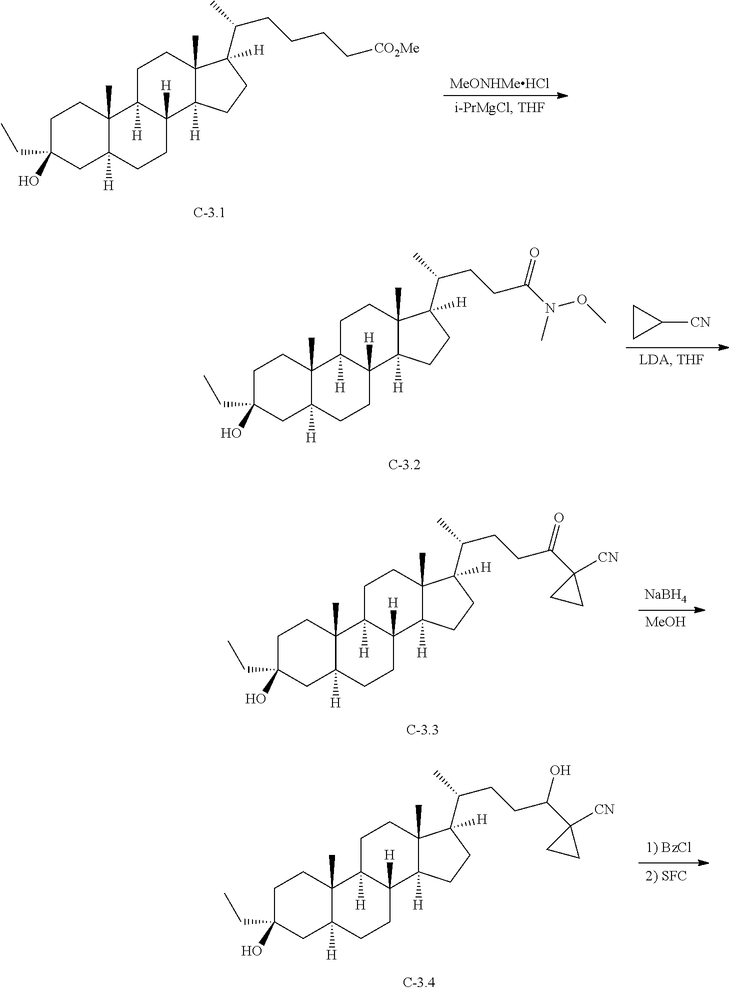

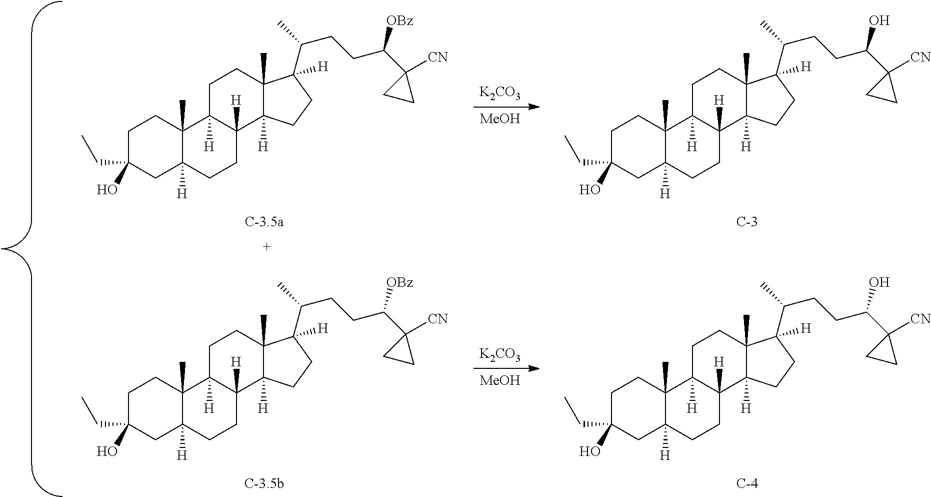

### Synthesis of C-3.2

To a suspension of (R)-methyl 4-((3S,5S,8R,9S,10S,13R,14S,17R)-3-ethyl-3-hydroxy-10,13-dimethylhexadecahydro-1H-cyclopenta[a]phenanthren-17-yl)pentanoate, C-3.1 (1 g, 2.38 mmol) and MeNHOMe HCl (347 mg, 3.56 mmol) in THF (10 mL) was added i-PrMgCl (2 M in THF, 4.76 mL, 9.52 mmol) at 0° C. under N₂. The mixture was stirred at 20° C. for 10 minutes then added to NH₄Cl (10% aq., 50 mL). The aqueous layer was extracted with DCM (2×50 mL). The combined organic layer was washed with brine (50 mL), dried over Na₂SO₄, filtered, and concentrated to give C-3.2 (980 mg, 92%). ¹H NMR (400 MHz, CDCl₃) δ_{H} 3.69 (s, 3H), 3.17 (s, 3H), 2.51-2.20 (m, 2H), 1.99-1.92 (m, 1H), 1.83-1.75 (m, 1H), 1.70-1.48 (m, 5H), 1.46-0.95 (m, 20H), 0.93 (d, J=6.4 Hz, 3H), 0.90-0.85 (m, 5H), 0.82 (s, 3H), 0.65 (s, 3H).

### Synthesis of C-3.3

To a solution of i-Pr₂NH (1.55 mL, 11.1 mmol) in THF (10 mL) under N₂ was added n-BuLi (4.00 mL, 2.5 M, 10.0 mmol) at −60° C. The mixture was stirred at −60° C. for 10 minutes and 0° C. for 10 minutes. To the mixture was added cyclopropanecarbonitrile (744 mg, 11.1 mmol) at −60° C. under N₂. The mixture was stirred at −60° C. under N₂ for 1 hour. A solution of C-3.2 (1 g, 2.23 mmol) in THF (5 mL) was added to the mixture at −60° C. under N₂ and the mixture was warmed to room temperature and aqueous NH₄Cl (50 mL) was added. The aqueous phase was extracted with DCM (2×50 mL). The combined organic phase was washed with brine (2×100 mL), dried over anhydrous Na₂SO₄, and concentrated. The residue was purified by silica gel chromatography (0-40% EtOAc in PE) to give C-3.3 (980 mg, 97%) as a colorless. ¹H NMR (400 MHz, CDCl₃) δ_{H} 3.01-2.73 (m, 2H), 1.97-1.89 (m, 1H), 1.87-1.71 (m, 3H), 1.70-1.48 (m, 11H), 1.48-0.94 (m, 17H), 0.92 (d, J=6.8 Hz, 3H), 0.86 (t, J=7.6 Hz, 3H), 0.80 (s, 3H), 0.63 (s, 3H).

### Synthesis of C-3.4

To a solution of C-3.3 (390 mg, 0.859 mmol) in MeOH (5.0 mL) was added NaBH₄ (50 mg, 1.32 mmol) at 20° C. and the mixture was stirred for 2 hours. The residue was poured into aqueous NH₄Cl (30 mL) and the aqueous phase was extracted with EtOAc (3×20 mL). The combined organic phase was washed with brine (2×30 mL), dried over anhydrous Na₂SO₄, filtered, and concentrated. The residue was purified by flash column (0˜40% of EtOAc in PE) to give C-3.4 (360 mg, 92%). ¹H NMR (400 MHz, CDCl₃) δ_{H} 3.06-2.89 (m, 1H), 2.00-1.91 (m, 1H), 1.88-1.64 (m, 5H), 1.64-1.57 (m, 6H), 1.55-1.31 (m, 8H), 1.29-1.16 (m, 8H), 1.15-0.98 (m, 7H), 0.97-0.93 (m, 4H), 0.88 (t, J=7.2 Hz, 3H), 0.82 (s, 3H), 0.65 (s, 3H).

### Synthesis of C-3.5a & C-3.5b

To a mixture of C-3.4 (170 mg, 0.373 mmol) and DMAP (45.5 mg, 0.373 mmol) in pyridine (3.0 mL) benzoyl chloride (104 mg, 0.746 mmol) was added dropwise at 20° C. The mixture was stirred at 70° C. for 12 hours. The mixture was cooled and then poured into ice-water (20 mL). The aqueous phase was extracted with EtOAc (3×30 mL). The combined organic phase was washed with brine (50 mL), dried over anhydrous Na₂SO₄, filtered, concentrated. Purification by flash column chromatography (0˜15% of EtOAc in PE) and further separation by SFC (Column: DAICEL CHIRALPAK AD (250 mm*30 mm, 10 um; Condition: 0.1% NH₃H₂O ETOH; Begin B: 30%; End B: 30%; Gradient Time (min): 100% B Hold Time (min); FlowRate (mL/min): 70; Injections: 60) provided C-3.5a (130 mg) and C-3.5b (140 mg).

C-3.5a: ¹H NMR (400 MHz, CDCl₃) δ_{H} 8.12-8.06 (m, 2H), 7.63-7.57 (m, 1H), 7.51-7.44 (m, 2H), 4.52-4.41 (m, 1H), 2.19-2.08 (m, 1H), 1.99-1.91 (m, 1H), 1.86-1.73 (m, 2H), 1.69-1.57 (m, 5H), 1.53-1.32 (m, 10H), 1.31-1.15 (m, 11H), 1.13-1.03 (m, 5H), 0.95 (d, J=6.4 Hz, 3H), 0.88 (t, J=7.6 Hz, 3H), 0.82 (s, 3H), 0.63 (s, 3H).

C-3.5b ¹H NMR (400 MHz, CDCl₃) δ_{H} 8.23-8.02 (m, 2H), 7.63-7.56 (m, 1H), 7.50-7.44 (m, 2H), 4.49-4.43 (m, 1H), 2.08-1.90 (m, 3H), 1.82-1.72 (m, 1H), 1.70-1.56 (m, 6H), 1.55-1.33 (m, 9H), 1.32-1.15 (m, 10H), 1.14-0.98 (m, 6H), 0.96 (d, J=6.8 Hz, 3H), 0.88 (t, J=7.6 Hz, 3H), 0.82 (s, 3H), 0.62 (s, 3H).

### Synthesis of C-3

To a mixture of C-3.5a (115 mg, 0.205 mmol) in MeOH (3.0 mL) was added K₂CO₃ (56.5 mg, 0.410 mmol) at 20° C. and the mixture was stirred at 20° C. for 48 hours. The mixture was poured into H₂O (20 mL) and the aqueous phase was extracted with EtOAc (3×20 mL). The combined organic phase was washed with brine (2×30 mL), dried over anhydrous Na₂SO₄, filtered, and concentrated to give C-3 (90 mg, 96%). ¹H NMR (400 MHz, CDCl₃) SH 3.09-2.95 (m, 1H), 1.99-1.92 (m, 1H), 1.87-1.75 (m, 2H), 1.70-1.57 (m, 7H), 1.56-1.43 (m, 5H), 1.43-1.19 (m, 12H), 1.19-0.95 (m, 8H), 0.94 (d, J=6.8 Hz, 3H), 0.93-0.90 (m, 1H), 0.88 (t, J=7.2 Hz, 3H), 0.82 (s, 3H), 0.65 (s, 3H). LC-ELSD/MS purity>99%, MS ESI calcd. for C₃₀H₄₉NO₂ [M−H₂O+H]⁺ 438.4, found 438.4.

### Synthesis of C-4

To a mixture of C-3.5b (125 mg, 0.223 mmol) in MeOH (3.0 mL) was added K₂CO₃ (61.5 mg, 0.446 mmol) at 20° C. The mixture was stirred at 20° C. for 48 hours. The mixture was poured into H₂O (10 mL) and the aqueous phase was extracted with EtOAc (3×20 mL). The combined organic phase was washed with saturated brine (2×30 mL), dried over anhydrous Na₂SO₄, filtered, and concentrated to give C-4 (80 mg, 80%). ¹H NMR (400 MHz, CDCl₃) δ_{H} 3.02-2.92 (m, 1H), 1.99-1.92 (m, 1H), 1.91-1.75 (m, 2H), 1.70-1.56 (m, 9H), 1.55-1.18 (m, 16H), 1.18-0.97 (m, 8H), 0.95 (d, J=6.8 Hz, 3H), 0.88 (t, J=7.2 Hz, 3H), 0.82 (s, 3H), 0.65 (s, 3H). LC-ELSD/MS purity>99%, MS ESI calcd. for C₃₀H₄₉NO₂ [M−H₂O+H]⁺ 438.4, found 438.4.

### Example 36: Synthesis of (2R,3R,6R)-2-ethyl-6-((3S,5S,8R,9S,10S,13R,14S,17R)-3-ethyl-3-hydroxy-10,13-dimethylhexadecahydro-1H-cyclopenta[a]phenanthren-17-yl)-3-hydroxyheptanenitrile (C-5) & (2R,3S,6R)-2-ethyl-6-((3S,5S,8R,9S,10S,13R,14S,17R)-3-ethyl-3-hydroxy-10,13-dimethylhexadecahydro-1H-cyclopenta[a]phenanthren-17-yl)-3-hydroxyheptanenitrile (C-6) & (2S,3R,6R)-2-ethyl-6-((3S,5S,8R,9S,10S,13R,14S,17R)-3-ethyl-3-hydroxy-10,13-dimethylhexadecahydro-1H-cyclopenta[a]phenanthren-17-yl)-3-hydroxyheptanenitrile (C-7) & (25,3S,6R)-2-ethyl-6-((3S,5S,8R,9S,10S,13R,14S,17R)-3-ethyl-3-hydroxy-10,13-dimethylhexadecahydro-1H-cyclopenta[a]phenanthren-17-yl)-3-hydroxyheptanenitrile (C-8)

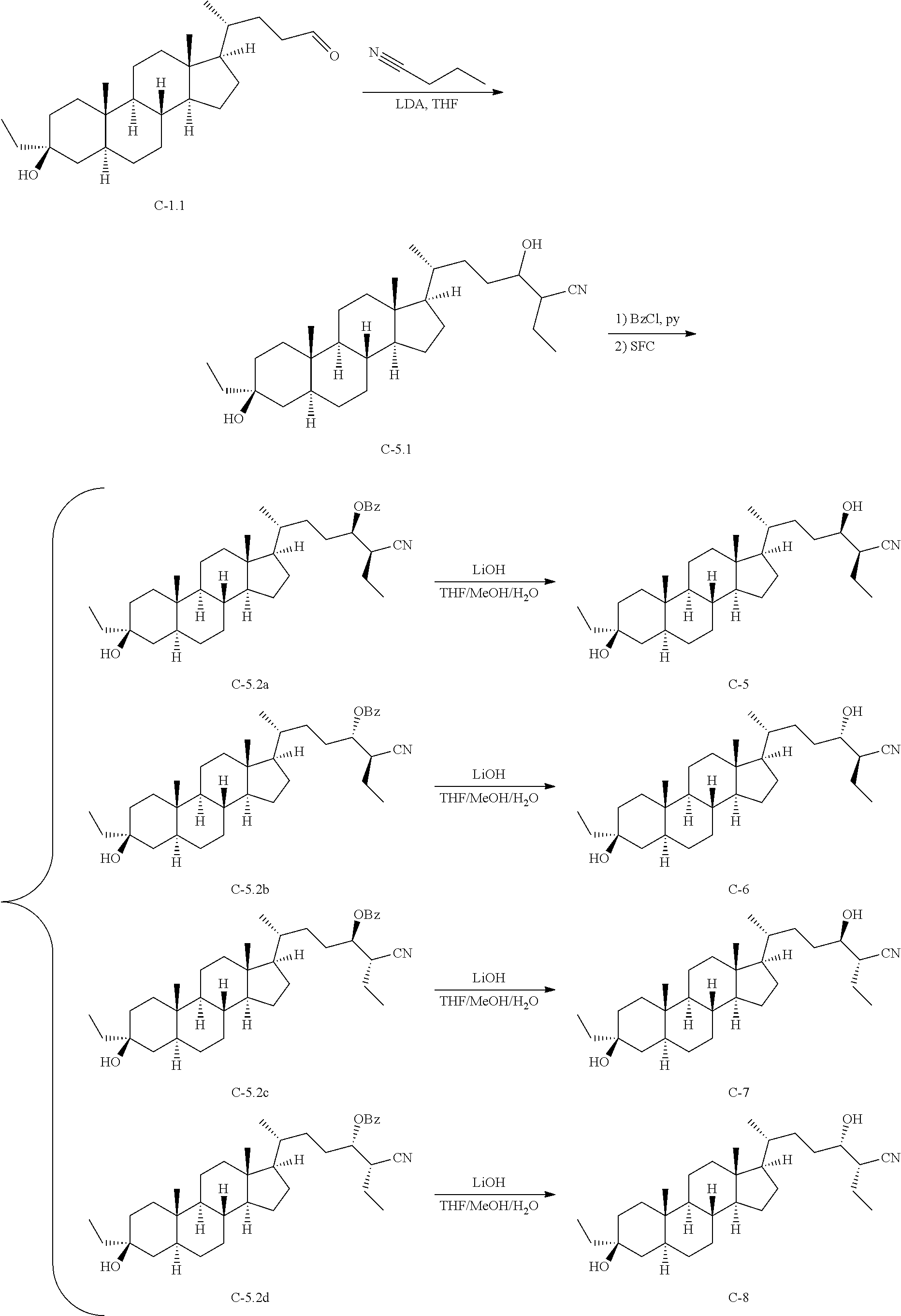

### Synthesis of C-5.1

To a solution of LDA (6.90 mL, 2 M, 13.8 mmol) in THF (20 mL) at −70° C. under N₂ was added dropwise a suspension of butanenitrile (1.27 g, 18.5 mmol). The mixture was stirred for 30 minutes at −70° C. A solution of C-1.1 (1.8 g, 4.63 mmol) was added dropwise at −70° C. The mixture was stirred for another 30 mins and then warmed to 25° C. gradually. After stirring at 25° C. for 4 hours, the reaction mixture was quenched by saturated NH₄Cl (40 mL) and extracted with EtOAc (3×80 mL). The combined organic phase was washed with brine (60 mL), dried over Na₂SO₄, filtered, and concentrated. Purification by combi-flash (0-20% of EtOAc in PE) provided C-5.1 (1.1 g).

### Synthesis of C-5.2a, C-5.2b, C-5.2c & C-5.2d

To a solution of C-5.1 (1.1 g, 2.40 mmol) in pyridine (20 mL) was added benzoyl chloride (504 mg, 3.59 mmol). After stirring at 25° C. for 16 hours, the reaction mixture was quenched with saturated NH₄Cl (40 mL) and extracted with EtOAc (3×80 mL). The combined organic phase was washed with brine (60 mL), dried over Na₂SO₄, filtered, and concentrated. Purification by combi-flash (0-20% of EtOAc in PE) and further separation by SFC (column: DAICEL CHIRALPAK AD (250 mm*30 mm, 10 um)), gradient: 50-50% B (A=0.1% NH₃/H₂O, B=MeOH), flow rate: 80 mL/min) provided C-5.2a (200 mg, Rt=1.911 min), C-5.2c (200 mg, Rt=2.626 min) and a mixture of C-5.2d & C-5.2b (Rt=2.119 min). The mixture of C-5.2d & C-5.2b was further separated by SFC (column: DAICEL CHIRALPAK AD (250 mm*30 mm, 10 um)), gradient: 30-30% B (A=0.1% NH₃/H₂O, B=EtOH), flow rate: 70 mL/min) to give C-5.2d (200 mg, Rt=1.428 min) and C-5.2b (200 mg, Rt=1.510 min). The configurations of C-24 and C-25 were randomly assigned.

### Synthesis of C-5

To a solution of C-5.2a (200 mg, 0.355 mmol) in THF (5.7 mL) and MeOH (2.8 mL) was added water (1.4 mL) and LiOH·H₂O (1.91 g, 50.0 mmol). After stirring at 25° C. for 16 hours, the mixture was added to water (10 mL) and extracted with EtOAc (20 mL). The organic layer was separated, dried over Na₂SO₄, filtered, and concentrated. Purification by combi-flash (0-15% EtOAc in PE) provided C-5 (30.2 mg, 18%). ¹H NMR (400 MHz, CDCl₃) δ_{H} 3.70-3.55 (m, 1H), 2.55-2.50 (m, 1H), 1.96-1.94 (m, 1H), 1.87-1.76 (m, 2H), 1.74-1.63 (m, 3H), 1.61-1.58 (m, 6H), 1.50-1.38 (m, 5H), 1.37-1.28 (m, 3H), 1.28-1.16 (m, 6H), 1.15-1.04 (m, 7H), 1.02-0.96 (m, 2H), 0.94-0.85 (m, 7H), 0.82 (s, 3H), 0.70-0.60 (m, 4H). LC-ELSD/MS purity>99%, MS ESI calcd. for C₃₀H₅₁NO₂ [M−H₂O+H]⁺ 440.4 found 440.4.

### Synthesis of C-6

To a solution of C-5.2b (200 mg, 0.355 mmol) in THF (5.7 mL) and MeOH (2.8 mL) was added water (1.4 mL) and LiOH·H₂O (1.91 g, 50.0 mmol). After stirring at 25° C. for 16 hours, the mixture was added to water (10 mL) and extracted with EtOAc (20 mL). The organic layer was separated, dried over Na₂SO₄, filtered, and concentrated. Purification by combi-flash (0-15% EtOAc in PE) provided C-6 (38.9 mg, 24%). ¹H NMR (400 MHz, CDCl₃) δ 3.70-3.60 (m, 1H), 2.65-2.55 (m, 1H), 1.96-1.94 (m, 1H), 1.77-1.75 (m, 3H), 1.72-1.66 (m, 3H), 1.61-1.55 (m, 5H), 1.46-1.44 (m, 2H), 1.44-1.32 (m, 5H), 1.32-1.19 (m, 5H), 1.19-1.17 (m, 1H), 1.16-1.07 (m, 7H), 1.03-1.01 (m, 2H), 0.96-0.85 (m, 8H), 0.83 (s, 3H), 0.70-0.60 (m, 4H). LC-ELSD/MS purity>99%, MS ESI calcd. for C₃₀H₅₁NO₂ [M−H₂O+H]⁺ 440.4, found 440.4.

### Synthesis of C-7

To a solution of C-5.2c (200 mg, 0.355 mmol) in THF (5.7 mL) and MeOH (2.8 mL) was added water (1.4 mL) and LiOH· H₂O (1.91 g, 50.0 mmol). After stirring at 25° C. for 16 hours, the mixture was added to water (10 mL) and extracted with EtOAc (20 mL). The organic layer was separated, dried over Na₂SO₄, filtered, and concentrated. Purification by combi-flash (0-15% EtOAc in PE) provided C-7 (70.1 mg, 43%). ¹H NMR (400 MHz, CDCl₃) δ 3.75-3.65 (m, 1H), 2.65-2.55 (m, 1H), 1.97-1.95 (m, 1H), 1.85-1.80 (m, 1H), 1.69-1.63 (m, 3H), 1.62-1.55 (m, 7H), 1.49-1.47 (m, 4H), 1.40-1.32 (m, 3H), 1.29-1.17 (m, 6H), 1.14-1.10 (m, 5H), 1.09-0.96 (m, 4H), 0.94-0.92 (m, 3H), 0.90-0.86 (m, 4H), 0.83 (s, 3H), 0.83-0.82 (m, 1H), 0.70-0.60 (m, 4H). LC-ELSD/MS purity>99%, MS ESI calcd. For C₃₀H₅₁NO₂ [M−H₂O+H]⁺ 440.4 found 440.4.

### Synthesis of C-8

To a solution of C-5.2d (200 mg, 0.355 mmol) in THF (5.7 mL) and MeOH (2.8 mL) was added water (1.4 mL) and LiOH·H₂O (1.91 g, 50.0 mmol). After stirring at 25° C. for 16 hours, the mixture was added to water (10 mL) and extracted with EtOAc (20 mL). The organic layer was separated, dried over Na₂SO₄, filtered, and concentrated. Purification by combi-flash (0-15% EtOAc in PE) provided C-8 (31.2 mg, 19%).

¹H NMR (400 MHz, CDCl₃) δ 3.65-3.60 (m, 1H), 2.55-2.50 (m, 1H), 1.96-1.94 (m, 1H), 1.87-1.76 (m, 2H), 1.74-1.63 (m, 3H), 1.61-1.58 (m, 6H), 1.50-1.38 (m, 5H), 1.37-1.28 (m, 3H), 1.28-1.16 (m, 6H), 1.15-1.04 (m, 7H), 1.02-0.96 (m, 2H), 0.94-0.85 (m, 7H), 0.82 (s, 3H), 0.70-0.60 (m, 4H). LC-ELSD/MS purity>99%, MS ESI calcd. For C₃₀H₅₁NO₂ [M−H₂O+H]⁺ 440.4 found 440.4.

### Example 37: (3R,6R)-3-hydroxy-6-((3S,5S,8R,9S,10S,13R,14S,17R)-3-hydroxy-3-(methoxymethyl)-10,13-dimethylhexadecahydro-1H-cyclopenta[a]phenanthren-17-yl)-2,2-dimethylheptanenitrile (C-9)

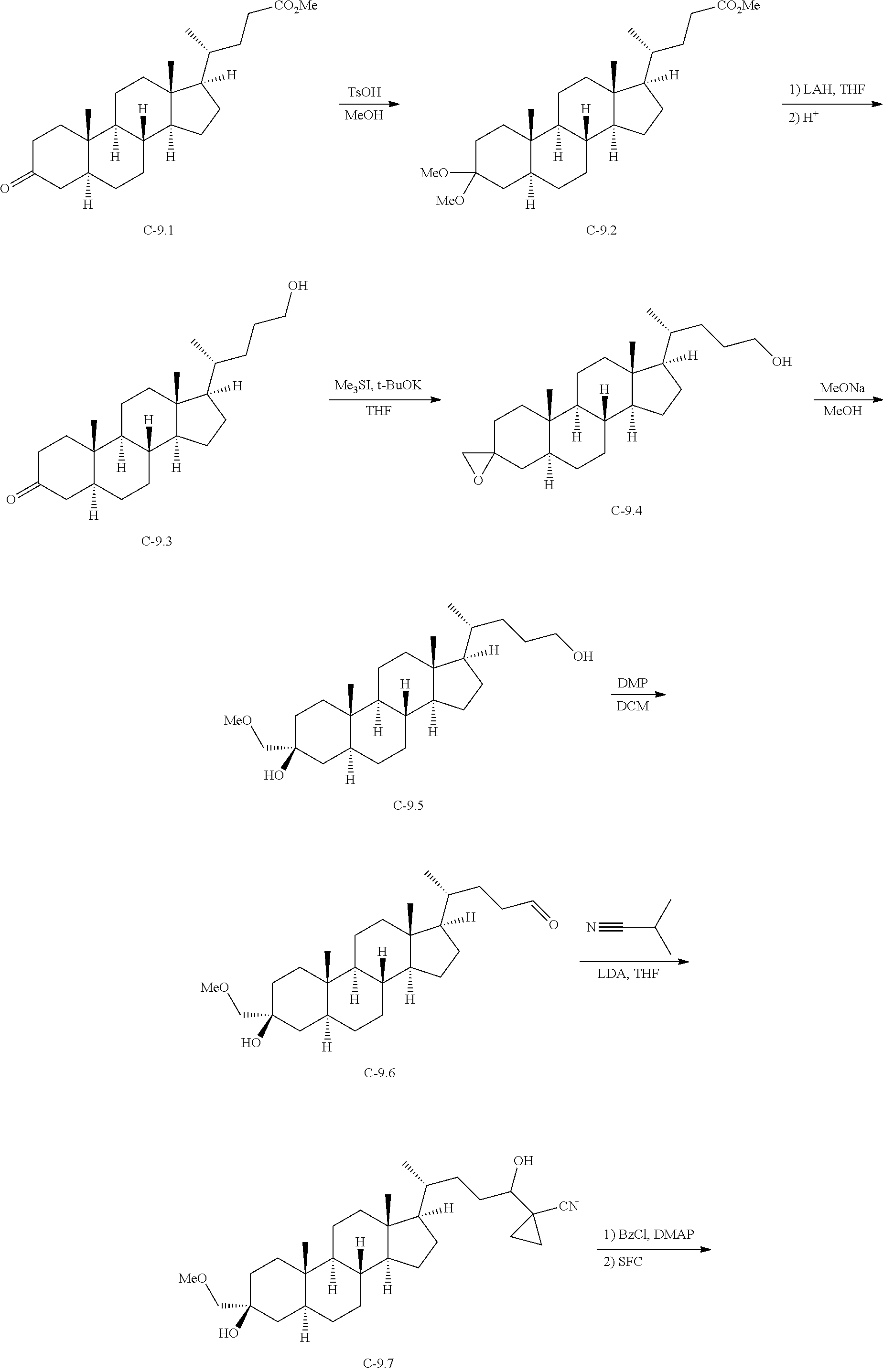

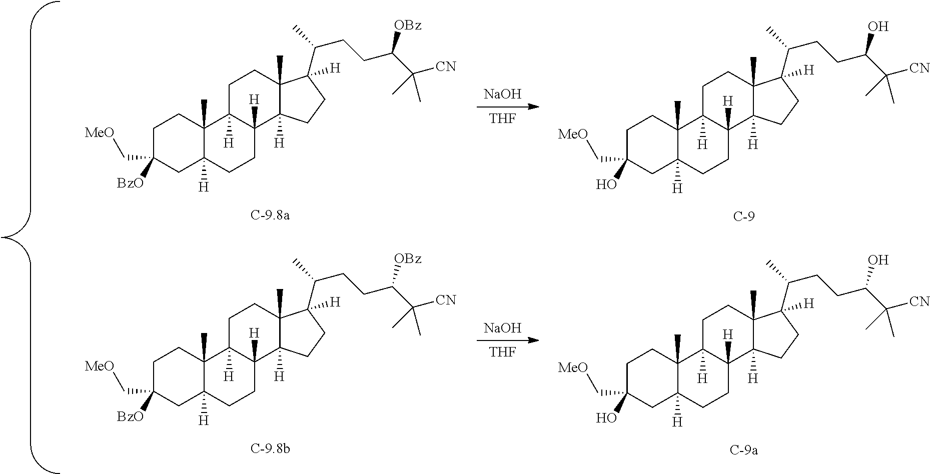

### Synthesis of C-9.2

To a solution of (R)-methyl 4-((5S,8R,9S,10S,13R,14S,17R)-10,13-dimethyl-3-oxohexadecahydro-1H-cyclopenta[a]phenanthren-17-yl)pentanoate, C-9.1 (15 g, 38.6 mmol; preparation described in WO2018/075699A1) in MeOH (50 mL) was added TsOH (1.32 g, 7.72 mmol) at 25° C. under N₂. After stirring at 55° C. for 16 hours, the mixture was treated with Et₃N (30 mL), and filtered to afford C-9.2 (15 g). ¹H NMR (400 MHz, CDCl₃) δ_{H} 3.66 (s, 3H), 3.19 (s, 3H), 3.14 (s, 3H), 2.40-2.29 (m, 1H), 2.26-2.15 (m, 1H), 1.98-1.57 (m, 7H), 1.56-0.97 (m, 14H), 0.95-0.90 (m, 4H), 0.91 (m, 3H), 0.78 (s, 3H), 0.76-0.66 (m, 1H), 0.64 (s, 3H).

### Synthesis of C-9.3

To a solution of C-9.2 (14 g, 32.2 mmol) in THF (150 mL) was added LiAlH₄ (2.44 g, 64.4 mmol) in one portion. After stirring at 25° C. for 1 hour, HCl (1M, 100 mL) was added to the mixture. After stirring at 15° C. for 0.5 hour, the mixture was filtered and concentrated to give C-9.3 (14 g).

### Synthesis of C-9.4

To a stirred solution of Me₃SI (11 g, 54.3 mmol) in THF (20 mL) was added t-BuOK (5.20 g, 46.5 mmol) at 0° C. for 1.0 hour under N₂. To the mixture was added C-9.3 (14 g, 38.8 mmol) in THF (30 mL) at 0° C. and the mixture was stirred at 25° C. for 2 hours. The reaction was treated with water (20 mL) and extracted with EtOAc (2×30 mL). The combined organic phase was washed with water (2×50 mL), brine (50 mL), dried over anhydrous Na₂SO₄, filtered, and concentrated to give C-9.4 (8 g). ¹H NMR (400 MHz, CDCl₃) δ_{H} 3.65-3.57 (m, 2H), 2.63-2.54 (m, 2H), 2.05-1.58 (m, 12H), 1.56-0.98 (m, 16H), 0.96-0.90 (m, 3H), 0.89-0.78 (m, 3H), 0.76-0.64 (m, 4H).

### Synthesis of C-9.5

To a solution C-9.4 (14 g, 37.3 mmol) in MeOH (60 mL) was added sodium methoxide (5.99 g) at 60° C. After stirring at 60° C. for 16 hours, water (200 mL) was added and the reaction mixture was concentrated to remove most of the solvent and extracted with EtOAc (2×300 mL). The combined organic phase was washed with brine (300 mL), dried over anhydrous Na₂SO₄, filtered, and concentrated to give C-9.5 (15 g). ¹H NMR (400 MHz, CDCl₃) δ_{H} 3.66-3.56 (m, 2H), 3.45-3.35 (m, 5H), 2.00-1.90 (m, 1H), 1.88-1.58 (m, 10H), 1.56-0.85 (m, 18H), 0.82 (s, 3H), 0.74 (s, 3H), 0.70-0.60 (m, 4H).

### Synthesis of C-9.6

To a solution of C-9.5 (12 g, 29.5 mmol) in DCM (140 mL) was added DMP (25 g, 59.0 mmol). After stirring at 35° C. for 30 min, the reaction mixture was diluted with DCM (100 mL), filtered, and concentrated. Purification by silica gel chromatography (0-15% EtOAc in PE) provided C-9.6 (8 g).

### Synthesis of C-9.7

To a solution of LDA (3.7 mL, 2 M, 7.41 mmol) in THF (10 mL), isobutyronitrile (511 mg, 7.41 mmol) was added dropwise at −20° C. under N₂. After stirring at −70° C. for 1 hour, a solution of C-9.6 (1 g, 2.47 mmol) in THF (20 mL) was added dropwise at −70° C. The mixture was stirred for another 30 mins and then warmed to 25° C. gradually, quenched by NH₄Cl (50 mL), and extracted with EtOAc (2×100 mL). The combined organic phase was dried over Na₂SO₄, filtered, and concentrated. The residue was purified by flash column (15-30% of PE in EtOAc) to provide C-9.7 (1.2 g).

### Synthesis of C-9.8a & C-9.8b

To a solution of C-9.7 (1.2 g, 2.53 mmol) in pyridine (5 mL) was added DMAP (13.6 mg, 0.252 mmol) and BzCl (1.06 g, 7.58 mmol) at 25° C. After stirring at 25° C. for 16 hours, the reaction mixture was diluted with water (20 mL) and extracted with EtOAc (2×50 mL). The combined organic phase was washed with brine (20 mL), dried over anhydrous Na₂SO₄, filtered, and concentrated. The residue was purified by flash column chromatography (0˜15% of EtOAc in PE) then further purified by SFC (column: DAICEL CHIRALPAK AD-H (250 mm*30 mm, 5 um)), gradient: 40-40% B (A=0.1% NH₃/H₂O, B=EtOH), flow rate: 50 mL/min) to provide C-9.8a (350 mg, 46.9%) and C-9.8b (350 mg, 46.9%). The configuration of C-24 was randomly assigned.

### Synthesis of C-9

To a solution of C-9.8a (350 mg, 0.513 mmol) in THF (5 mL) was added NaOH (24.4 mg, 1.02 mmol) at 25° C. After stirring at 25° C. for 15 min under N₂, the reaction mixture was quenched with saturated NH₄Cl (20 mL) and extracted with EtOAc (3×20 mL). The combined organic layer was washed with brine (50 mL), dried over Na₂SO₄, filtered, and concentrated to give C-9. Purification by silica gel column (0-15% of EtOAc in PE) afforded C-9 (50 mg, 24.1%). ¹H NMR (400 MHz, CDCl₃) δ 3.45-3.35 (m, 5H), 2.50 (s, 1H), 2.00-1.92 (m, 1H), 1.88-1.75 (m, 1H), 1.75-1.42 (m, 17H), 1.41-0.85 (m, 19H), 0.83 (s, 3H), 0.70-0.60 (m, 4H). LC-ELSD/MS purity>99%, MS ESI calcd. for C₃₀H₅₁NO₃ [M+H−H₂O]⁺ 456, found 456.

### Synthesis of C-9a

To a solution of C-9.8b (350 mg, 0.513 mmol) in THF (5 mL) was added NaOH (24.4 mg, 1.02 mmol) at 25° C. After stirring at 25° C. for 16 hours, the reaction mixture was quenched with saturated aqueous NH₄Cl (20 mL) and extracted with EtOAc (3×20 mL). The combined organic layer was washed with brine (50 mL), dried over Na₂SO₄, filtered, and concentrated to give C-9a. Purification by silica gel column (0-15% of EtOAc in PE) provided C-9a (10.1 mg, 4.15%). ¹H NMR (400 MHz, CDCl₃) δ 3.45-3.35 (m, 5H), 2.49 (s, 1H), 2.00-1.92 (m, 1H), 1.88-1.75 (m, 1H), 1.75-1.42 (m, 17H), 1.41-0.85 (m, 19H), 0.83 (s, 3H), 0.70-0.60 (m, 4H). LC-ELSD/MS purity>98%, MS ESI calcd. for C₃₀H₅₁NO₃ [M+H−H₂O]⁺ 456, found 456.

### Example 38: (3S,6R)-6-((3S,5S,8R,9S,10S,13R,14S,17R)-3-ethyl-3-hydroxy-10,13-dimethylhexadecahydro-1H-cyclopenta[a]phenanthren-17-yl)-3-hydroxyheptanenitrile (C-10) & (3R,6R)-6-((3S,5S,8R,9S,10S,13R,14S,17R)-3-ethyl-3-hydroxy-10,13-dimethylhexadecahydro-1H-cyclopenta[a]phenanthren-17-yl)-3-hydroxyheptanenitrile (C-11)

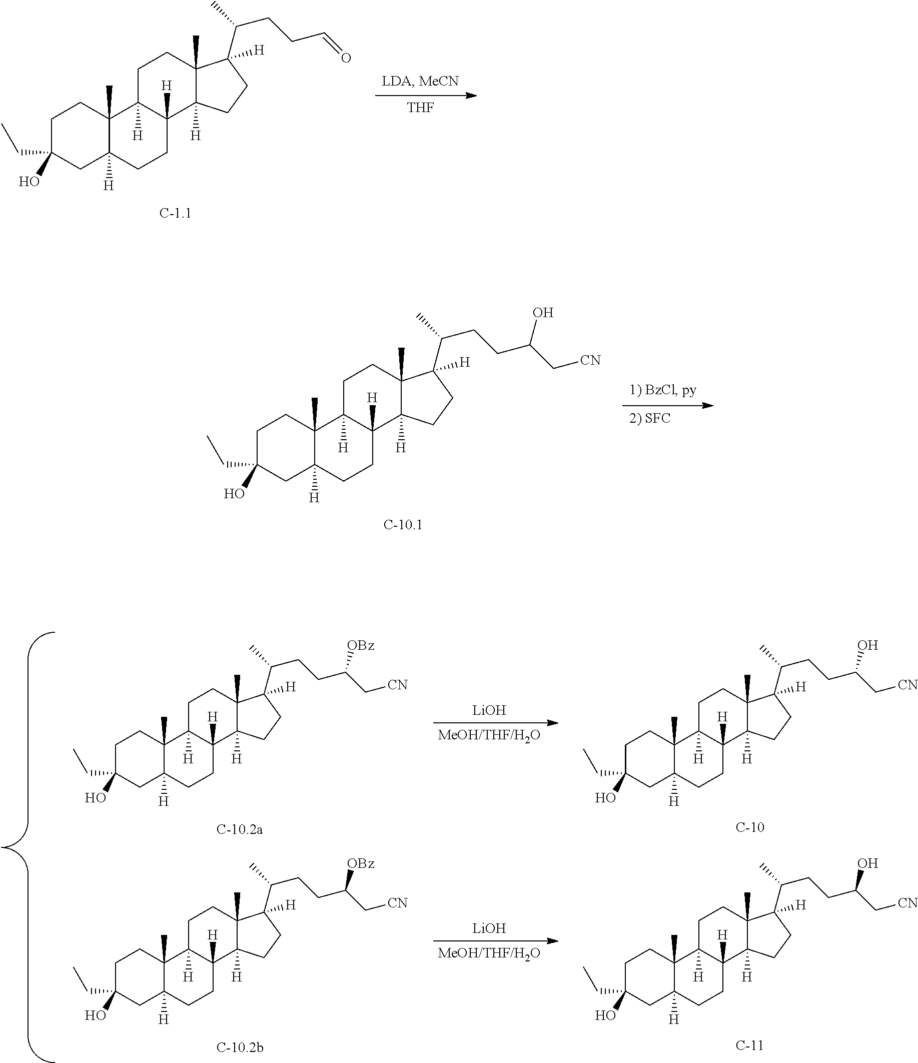

### Synthesis of C-10.1

To a solution of LDA (20.4 mL, 2 M, 40.8 mmol) in THF (30 mL) at −70° C. under N₂ a suspension of MeCN (1.67 g, 40.8 mmol) was added dropwise. The mixture was stirred for 30 minutes at −70° C. A solution of (R)-4-((3S,5S,8R,9S,10S,13R,14S,17R)-3-ethyl-3-hydroxy-10,13-dimethylhexadecahydro-1H-cyclopenta[a]phenanthren-17-yl)pentanal, C-1.1 (4 g, 10.2 mmol; preparation described in WO2018/075699A1) in THF (10 mL) was added dropwise at -70° C. The mixture was stirred for another 30 mins and then warmed to 25° C. gradually and stirred for 4 hours. The reaction mixture was quenched by saturated NH₄Cl (40 mL) and extracted with EtOAc (3×80 mL). The combined organic phase was washed with brine (60 mL), dried over Na₂SO₄, filtered, and concentrated. Purification by silica gel chromatography (EtOAc/PE=50/1-1/1) provided C-10.1 (2.4 g). ¹H NMR (400 MHz, CDCl₃) δ_{H} 3.96-3.79 (m, 1H), 2.63-2.42 (m, 2H), 2.01-1.58 (m, 8H), 1.50-1.36 (m, 8H), 1.30-1.03 (m, 11H), 1.02-0.77 (m, 13H), 0.68-0.59 (m, 4H).

### Synthesis of C-10.2a & C-10.2b

To a solution of C-10.1 (500 mg, 1.16 mmol) in pyridine (8 mL) was added BzCl (489 mg, 3.48 mmol) at 20° C. After stirring for 5 hours at 20° C., 1M HCl (10 mL) was added and the mixture extracted with EtOAc (3×20 mL). The organics were dried over anhydrous Na₂SO₄, and concentrated. Purification by silica gel chromatography provided product (400 mg). Further purification by SFC (Column: DAICEL CHIRALPAK AD-H (250 mm*30 mm, 5 um), Condition: 0.1% NH₃H₂O IPA, Begin B: 20%, End B: 20, FlowRate (mL/min): 50, Injections: 150) provided C-10.2a (150 mg, 37.5%) and C-10.2b (150 mg, 37.5%). The configuration of C-24 was randomly assigned.

### Synthesis of C-10

To a solution of C-10.2a (150 mg, 0.281 mmol) in MeOH/THF/H₂O (4:2:1) (7 mL) was added LiOH·H₂O (118 mg, 2.81 mmol) at 20° C. After stirring for 5 hours at 20° C., the mixture was diluted with water (10 mL) at 20° C. and extracted with EtOAc (3×10 mL). The mixture was dried over anhydrous Na₂SO₄ and concentrated. The residue was purified by silica gel chromatography to provide C-10 (21.4 mg, 17.8%). ¹H NMR (400 MHz, CDCl₃) δ_{H} 3.99-3.77 (m, 1H), 2.63-2.42 (m, 2H), 1.99-1.56 (m, 10H), 1.54-1.31 (m, 8H), 1.30-1.02 (m, 10H), 1.01-0.78 (m, 12H), 0.69-0.58 (m, 4H). LC-ELSD/MS purity>99%, MS ESI calcd. for C₂₈H₄₇NO₂ [M−H₂O+H]⁺ 412.4, found 412.4.

### Synthesis of C-11

To a solution of C-10.2b (150 mg, 0.281 mmol) in MeOH/THF/H₂O (4:2:1) (7 mL) was added LiOH·H₂O (118 mg, 2.81 mmol) at 20° C. After stirring for 5 hours at 20° C., the mixture was diluted with water (10 mL) and extracted with EtOAc (2×10 mL). The organics were dried over anhydrous Na₂SO₄ and concentrated. The residue was purified by silica gel chromatography to provide C-11 (17.9 mg, 14.9%). ¹H NMR (400 MHz, CDCl₃) δ_{H} 3.97-3.86 (m, 1H), 2.62-2.44 (m, 2H), 2.03-1.58 (m, 10H), 1.51-1.36 (m, 9H), 1.27-0.96 (m, 11H), 0.94-0.77 (m, 10H), 0.69-0.57 (m, 4H). LC-ELSD/MS purity>99%, MS ESI calcd. for C₂₈H₄₇NO₂ [M−H₂O+H]⁺ 412.4, found 412.4.

### Example 39: Synthesis of (2R,4aS,4bR,6aS,7R,10aS,10bR,12aS)-6a-methyl-7-((R)-1-(5-methyl-1H-tetrazol-1-yl)propan-2-yl)-2-propyloctadecahydrochrysen-2-ol (C-12) & (3S,6R)-6-((3S,8S,9S,10R,13R,14S,17R)-3-ethyl-3-hydroxy-10,13-dimethyl-2,3,4,7,8,9,10,11,12,13,14,15,16,17-tetradecahydro-1H-cyclopenta[a]phenanthren-17-yl)-3-hydroxy-2,2-dimethylheptanenitrile (C-13)

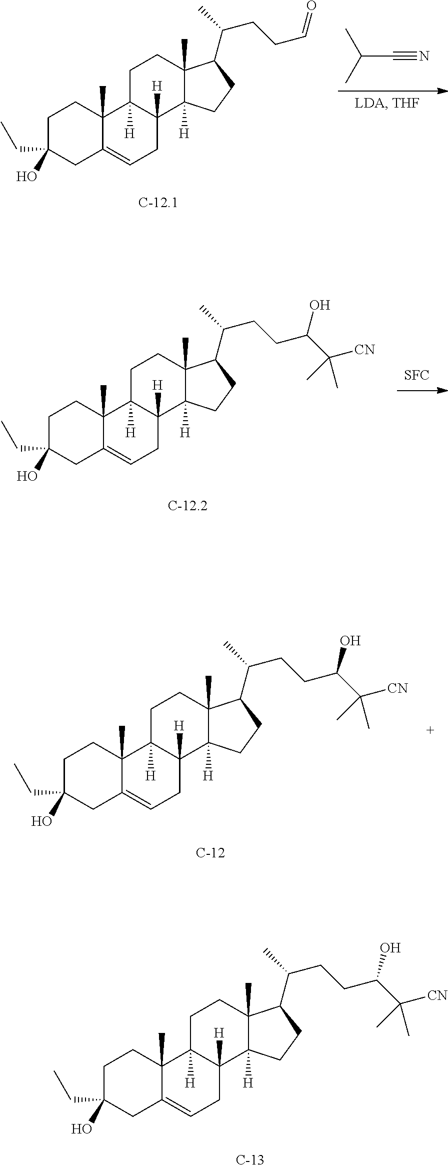

### Synthesis of C-12.2

To a solution of diisopropylamine (114 mg, 1.13 mmol) in THF (5 mL) was added n-BuLi (0.492 mL, 1.23 mmol, 2.5M in hexane) dropwise under N₂. After addition, the mixture was cooled to −70° C. Isobutyronitrile (78.0 mg, 1.13 mmol) was added and the reaction mixture was then warmed to −10° C. After 10 minutes, a solution of (R)-4-((3S,8S,9S,10R,13R,14S,17R)-3-ethyl-3-hydroxy-10,13-dimethyl-2,3,4,7,8,9,10,11,12, 13, 14, 15, 16, 17-tetradecahydro-1H-cyclopenta[a]phenanthren-17-yl)pentanal, C-12.1 (400 mg, 1.03 mmol; preparation described in WO2018/075699A1) in THF (10 mL) was added. The mixture was stirred at 25° C. for 1 hour. The mixture was quenched with saturated NH₄Cl (30 mL) and extracted with EtOAc (3×15 mL). The combined organic phase was dried over Na₂SO₄, filtered, and concentrated. The residue was purified by combi-flash (0-15% of EtOAc in PE) to give C-12.2 (130 mg, 28%). ¹H NMR (400 MHz, CDCl₃) δ 5.30-5.25 (m, 1H), 3.40-3.30 (m, 1H), 2.40-2.30 (m, 1H), 2.10-1.90 (m, 5H), 1.90-1.71 (m, 4H), 1.71-1.51 (m, 7H), 1.51-1.19 (m, 11H), 1.19-0.99 (m, 8H), 0.99-0.80 (m, 8H), 0.69 (s, 3H). LCMS purity>99%, MS ESI calcd. for C₃₀H₄₉NO₂ [M+H−H₂O]⁺ 438, found 438.

### Synthesis of C-12 & C-13

C-12.2 (550 mg, 1.20 mmol) was purified by SFC (column: AD (250 mm*30 mm, 10 um), gradient: 30-30% B (A=0.1% NH₃/H₂O, B=EtOH), flow rate: 60 mL/min) to give C-12 (73 mg, 13%) and C-13 (72 mg, 13%). The configuration of C-24 was randomly assigned.

C-12: ¹H NMR (400 MHz, CDCl₃) δ 5.30-5.25 (m, 1H), 3.40-3.30 (m, 1H), 2.40-2.30 (m, 1H), 2.10-1.90 (m, 3H), 1.90-1.69 (m, 2H), 1.69-1.60 (m, 4H), 1.51-1.40 (m, 6H), 1.40-1.23 (m, 10H), 1.23-1.06 (m, 4H), 1.06-0.92 (m, 9H), 0.92-0.80 (m, 3H), 0.68 (s, 3H). LC-ELSD/MS purity>99%, MS ESI calcd. For C₃₀H₄₉NO₂ [M+H−H₂O]⁺ 438, found 438.

C-13: ¹H NMR (400 MHz, CDCl₃) δ 5.30-5.25 (m, 1H), 3.40-3.30 (m, 1H), 2.40-2.30 (m, 1H), 2.10-1.90 (m, 3H), 1.90-1.71 (m, 5H), 1.71-1.60 (m, 2H), 1.51-1.40 (m, 6H), 1.40-1.23 (m, 10H), 1.23-1.01 (m, 9H), 1.01-0.91 (m, 5H), 0.91-0.80 (m, 3H), 0.68 (s, 3H). LC-ELSD/MS purity>99%, MS ESI calcd. For C₃₀H₄₉NO₂ [M+H−H₂O]⁺ 438, found 438.

### Example 40: Synthesis of 1-((1S,4R)-4-((3S,8S,9S,10R,13R,14S,17R)-3-ethyl-3-hydroxy-10,13-dimethyl-2,3,4,7,8,9,10,11,12,13,14,15,16,17-tetradecahydro-1H-cyclopenta[a]phenanthren-17-yl)-1-hydroxypentyl)cyclobutane-1-carbonitrile (C-18) and 1-((1R,4R)-4-((3S,8S,9S,10R,13R,14S,17R)-3-ethyl-3-hydroxy-10,13-dimethyl-2,3,4,7,8,9,10,11,12,13,14,15,16,17-tetradecahydro-1H-cyclopenta[a]phenanthren-17-yl)-1-hydroxypentyl)cyclobutane-1-carbonitrile (C-19)

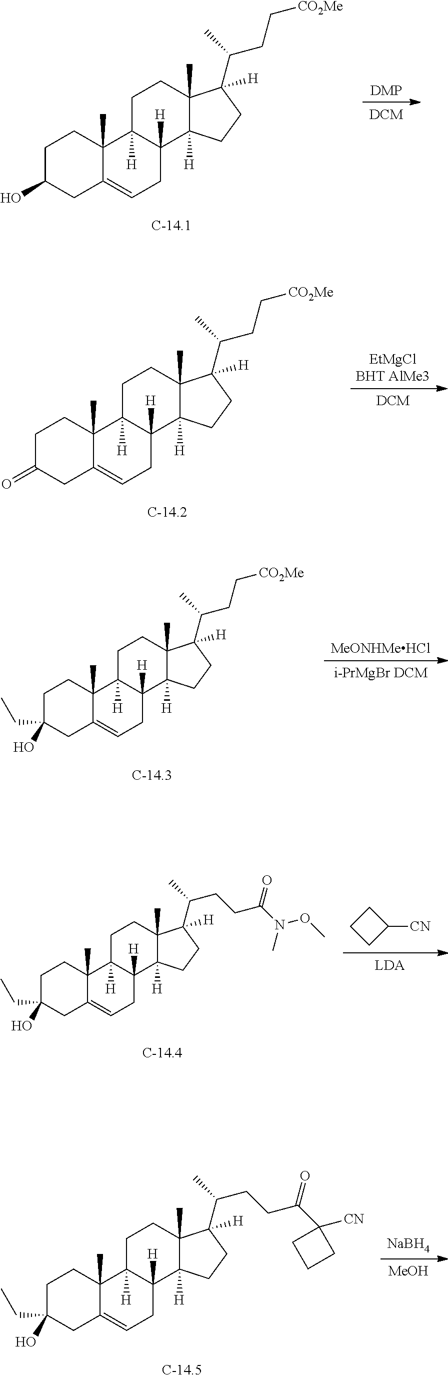

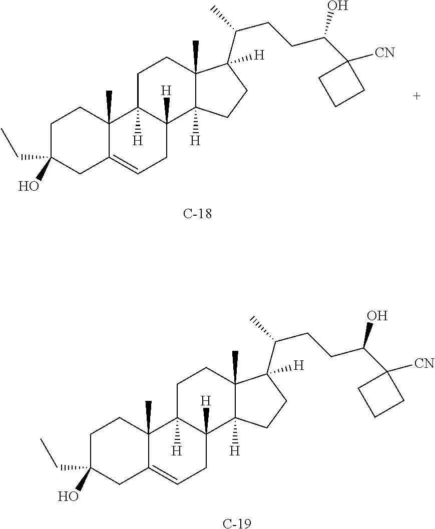

### Synthesis of C-14.2

To a solution of C-14.1 (10 g, 25.7 mmol; preparation described in Bioorg. Med. Chem. Lett. 2014, 24 (15), 3480-3485) in DCM (100 mL) was added DMP (43.2 g, 102 mmol) at 25° C. and the resulting mixture was stirred for 1 h. To the mixture was added NaHCO₃ (30 mL, sat. aq.) and Na₂S₂O₃ (30 mL, sat. aq.) and the aqueous phase was extracted with DCM (2×30 mL). The combined organic layers were washed with NaHCO₃/Na₂S₂O₃ (1:1, 2×40 mL, sat. aq.) and brine (30 mL), dried over anhydrous Na₂SO₄, filtered, and concentrated to give C-14.2 (10 g). ¹H NMR (400 MHz, CDCl₃) δ_{H} 5.36-5.30 (m, 1H), 3.65 (s, 3H), 3.36-3.20 (m, 1H), 2.90-2.73 (m, 1H), 2.55-2.14 (m, 7H), 1.94-1.38 (m, 16H), 1.17 (s, 3H), 0.92 (d, J=6.0 Hz, 3H), 0.70 (s, 3H).

### Synthesis of C-14.3

To a solution of BHT (11.3 g, 51.6 mmol) in toluene (35 mL) under N₂ at 0° C. was added AlMe₃ (2 M in toluene, 12.9 mL, 25.8 mmol) dropwise and the resulting mixture was stirred at 25° C. for 1 h. To the MAD solution (37.1 g, 77.3 mmol) was added C-14.2 (10 g, 25.8 mmol) in DCM (20 ml, 25.8 mmol) at -70° C. and the reaction mixture was stirred for 30 min. EtMgCl (9.21 g, 77.3 mmol) was added and the mixture was stirred for 2 h at −70° C. The reaction was quenched with citric acid (3×50 mL). The combined organic layers were washed with brine (2×50 mL), dried over anhydrous Na₂SO₄, filtered, and concentrated to give C-14.3 (10 g). ¹H NMR (400 MHz, CDCl₃) δ_{H} 5.32-5.25 (m, 1H), 3.66 (s, 3H), 2.40-2.33 (m, 2H), 2.33-2.16 (m, 2H), 1.92-1.62 (m, 7H), 1.54-1.29 (m, 10H), 1.21-1.05 (m, 5H), 1.03 (s, 3H), 1.01-0.94 (m, 2H), 0.92 (d, J=6.0 Hz, 3H), 0.85 (t, J=7.6 Hz, 3H), 0.67 (s, 3H).

### Synthesis of C-14.4

To a suspension of C-14.3 (1 g, 2.48 mmol) and MeONHMe·HCl (362 mg, 3.72 mmol) in THF (10 mL) was added i-PrMgCl (2 M in THF, 4.96 mL, 9.92 mmol) at 0° C. under N₂ and the resulting mixture was stirred at 20° C. for 10 min. To the mixture was added NH₄Cl (10% aq., 50 mL) and the aqueous layer was extracted with DCM (2×50 mL). The combined organic layers were washed with brine (50 mL), dried over anhydrous Na₂SO₄, filtered, and concentrated. The residue was purified by silica gel chromatography (0˜30% of EtOAc in PE) to give C-14.4 (580 mg, 53.2%). ¹H NMR (400 MHz, CDCl₃) δ_{H} 5.30-5.26 (m, 1H), 3.69 (s, 3H), 3.17 (s, 3H), 2.51-2.40 (m, 1H), 2.40-2.29 (m, 2H), 2.07-1.61 (m, 9H), 1.56-1.04 (m, 16H), 1.03 (s, 3H), 0.95 (d, J=6.4 Hz, 3H), 0.85 (t, J=7.6 Hz, 3H), 0.68 (s, 3H).

### Synthesis of C-14.5

To a solution of i-Pr₂NH (861 μL, 6.15 mmol) in THF (5 mL) under N₂ was added n-BuLi (2.23 mL, 2.5 M, 5.59 mmol) at −60° C. and the resulting mixture was stirred at −60° C. for 10 min and then at 0° C. for 10 min. Cyclobutanecarbonitrile (498 mg, 6.15 mmol) was added to freshly prepared LDA (6.15 mmol, in 5 ml THF) at −60° C. under N₂ and the mixture was stirred at −60° C. under N₂ for 1 h. A solution of C-14.4 (550 mg, 1.23 mmol) in THF (5 mL) was added to the mixture at −60° C. under N₂ and the mixture was warmed to room temperature. NH₄Cl (sat. aq., 50 mL) was added and the aqueous phase was extracted with DCM (2×50 mL). The combined organic layers were washed with brine (2×100 mL), dried over anhydrous Na₂SO₄, filtered, and concentrated to give C-14.5 (760 mg). The residue was purified by silica gel chromatography (0˜30% of EtOAc in PE) and lyophilized to give C-14.5 (12 mg, 80.5%). ¹H NMR (400 MHz, CDCl₃) δ_{H} 5.29 (d, J=5.6 Hz, 1H), 2.78-2.47 (m, 6H), 2.40-2.32 (m, 1H), 2.29-2.20 (m, 1H), 2.09-1.95 (m, 4H), 1.73-1.59 (m, 4H), 1.52-1.27 (m, 10H), 1.25 (s, 3H), 1.22-1.04 (m, 5H), 1.03 (s, 3H), 0.93 (d, J=6.4 Hz, 3H), 0.85 (t, J=7.6 Hz, 3H), 0.68 (s, 3H).

### Synthesis of C-18 and C-19

To a solution of C-14.5 (590 mg, 1.26 mmol) in MeOH (10 mL) was added NaBH₄ (47.9 mg, 1.26 mmol) at 20° C. and the resulting mixture was stirred for 20 min. The mixture was poured into NH₄Cl (20 mL) and was extracted with DCM (2×10 mL). The combined organic layers were washed with brine (2×10 mL), dried over anhydrous Na₂SO₄, filtered, and concentrated. The residue was purified by SFC (Column: DAICEL CHIRALPAK AD (250 mm*30 mm, 10 um); Condition: 0.1% NH₃H₂O ETOH; Begin B: 30; End B: 30; FlowRate (ml/min): 70; Injections: 60) to give C-18 (200 mg, 33.9%) and C-19 (190 mg, 32.2%).

C-18: ¹H NMR (400 MHz, CDCl₃) δ_{H} 5.38-5.17 (m, 1H), 3.64-3.45 (m, 1H), 2.54-1.62 (m, 18H), 1.58-1.06 (m, 17H), 1.03 (s, 3H), 0.95 (d, J=6.4 Hz, 3H), 0.85 (t, J=7.6 Hz, 3H), 0.68 (s, 3H). LC-ELSD/MS 30-90AB_2 min_E, purity>99%, MS ESI calcd. for C31H₄₈NO [M−H₂O+H]⁺ 450.4, found 450.4.

C-19: ¹H NMR (400 MHz, CDCl₃) δ_{H} 5.32-5.21 (m, 1H), 3.66-3.55 (m, 1H), 2.49-2.30 (m, 4H), 2.24-2.11 (m, 2H), 2.07-1.93 (m, 4H), 1.89-1.79 (m, 1H), 1.77-1.46 (m, 16H), 1.40-1.06 (m, 8H), 1.03 (s, 3H), 0.94 (d, J=6.4 Hz, 3H), 0.85 (t, J=7.6 Hz, 3H), 0.68 (s, 3H). LC-ELSD/MS 30-90AB_2 min_E, purity>99%, MS ESI calcd. for C₃₁H₄₈NO [M−H₂O+H]⁺ 450.4, found 450.4.

### Example 41: Synthesis of 1-((1S,4R)-4-((3S,8S,9S,10R,13R,14S,17R)-3-ethyl-3-hydroxy-10,13-dimethyl-2,3,4,7,8,9,10,11,12,13,14,15,16,17-tetradecahydro-1H-cyclopenta[a]phenanthren-17-yl)-1-hydroxypentyl)cyclopropane-1-carbonitrile (C-20) and 1-((1R,4R)-4-((3S,8S,9S,10R,13R,14S,17R)-3-ethyl-3-hydroxy-10,13-dimethyl-2,3,4,7,8,9,10,11,12,13,14,15,16,17-tetradecahydro-1H-cyclopenta[a]phenanthren-17-yl)-1-hydroxypentyl)cyclopropane-1-carbonitrile (C-21)

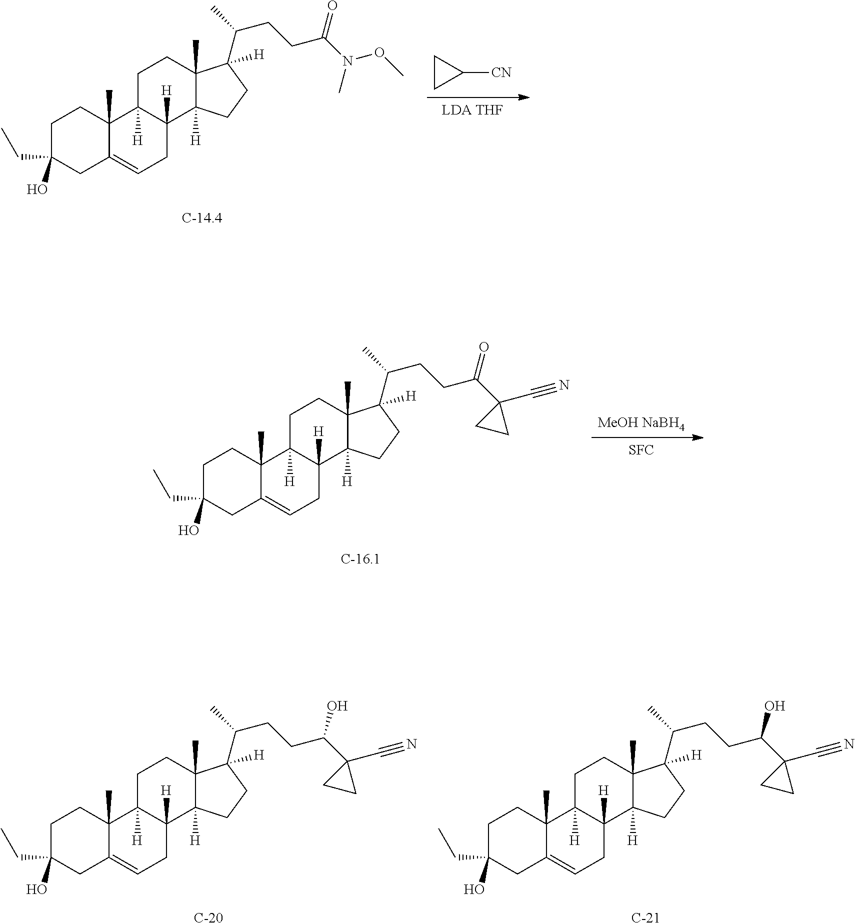

### Synthesis of C-16.1

Cyclopropanecarbonitrile (436 mg, 6.5 mmol) was added to freshly prepared LDA (5.6 mmol, in 5 ml THF) at −60° C. under N₂ and the resulting mixture was stirred at −60° C. under N₂ for 1 h. A solution of C-14.4 (580 mg, 1.3 mmol) in THF (5 mL) was added to the mixture at −60° C. under N₂ and the mixture was warmed to room temperature. NH₄Cl (sat. aq., 50 mL) was added and the aqueous phase was extracted with DCM (2×50 mL). The combined organic layers were washed with brine (2×100 mL), dried over anhydrous Na₂SO₄, filtered, and concentrated. The residue was purified by silica gel chromatography twice (0˜30% of EtOAc in PE) to give C-16.1 (590 mg, 98.9%). LC-ELSD/MS 30-90AB_2 min_E, purity>99%, MS ESI calcd. for C₃₀H₄NO [M−H₂O+H]⁺ 434.3, found 434.3.

### Synthesis of C-20 and C-21

To a solution of C-16.1 (590 mg, 1.34 mmol) in MeOH (100 mL) was added NaBH₄ (50.9 mg, 1.34 mmol) at 20° C. and the resulting mixture was stirred for 20 min. To the mixture was added saturated aqueous NH₄Cl (20 mL) and the mixture was extracted with DCM (2×10 mL). The combined organic layers were washed with brine (2×10 mL), dried over anhydrous Na₂SO₄, filtered, concentrated, and purified by SFC (Column: DAICEL CHIRALPAK AD (250 mm*30 mm, 10 um); Condition: 0.1% NH₃H₂O ETOH; Begin B: 25; End B: 25; FlowRate (ml/min): 70; Injections: 120) to give C-20 (160 mg, 27.1%) and C-21 (200 mg, 33.9%).

C-20: ¹H NMR (400 MHz, CDCl₃) δ_{H} 5.38-5.22 (m, 1H), 3.04-2.93 (m, 1H), 2.42-2.32 (m, 1H), 2.07-1.57 (m, 12H), 1.56-1.29 (m, 9H), 1.28-1.04 (m, 9H), 1.03 (s, 3H), 1.02-0.98 (m, 2H), 0.97 (d, J=6.4 Hz, 3H), 0.85 (t, J=7.6 Hz, 3H), 0.68 (s, 3H). LC-ELSD/MS 30-90AB_2 min_E, purity>99%, MS ESI calcd. for C₃₀H₄₆NO [M−H₂O+H]⁺ 436.3, found 436.3.

C-21: ¹H NMR (400 MHz, CDCl₃) δ_{H} 5.33-5.24 (m, 1H), 3.07-2.99 (m, 1H), 2.40-2.32 (m, 1H), 2.09-1.55 (m, 14H), 1.54-1.29 (m, 9H), 1.28-1.23 (m, 4H), 1.23-1.04 (m, 5H), 1.03 (s, 3H), 0.96 (d, J=6.8 Hz, 3H), 0.85 (t, J=7.6 Hz, 3H), 0.68 (s, 3H). LC-ELSD/MS 30-90AB_2 min_E, purity>99%, MS ESI calcd. for C₃₀H₄₆NO [M−H₂O+H]⁺ 436.4, found 436.4.

### Example 42:1-((1S,4R)-1-hydroxy-4-((3S,8S,9S,10R,13R,14S,17R)-3-hydroxy-3,10,13-trimethyl-2,3,4,7,8,9,10,11,12,13,14,15,16,17-tetradecahydro-1H-cyclopenta[a]phenanthren-17-yl)pentyl)cyclopropane-1-carbonitrile (C-22) and 1-((1R,4R)-1-hydroxy-4-((3S,8S,9S,10R,13R,14S,17R)-3-hydroxy-3,10,13-trimethyl-2,3,4,7,8,9,10,11,12,13,14,15,16,17-tetradecahydro-1H-cyclopenta[a]phenanthren-17-yl)pentyl)cyclopropane-1-carbonitrile (C-23)

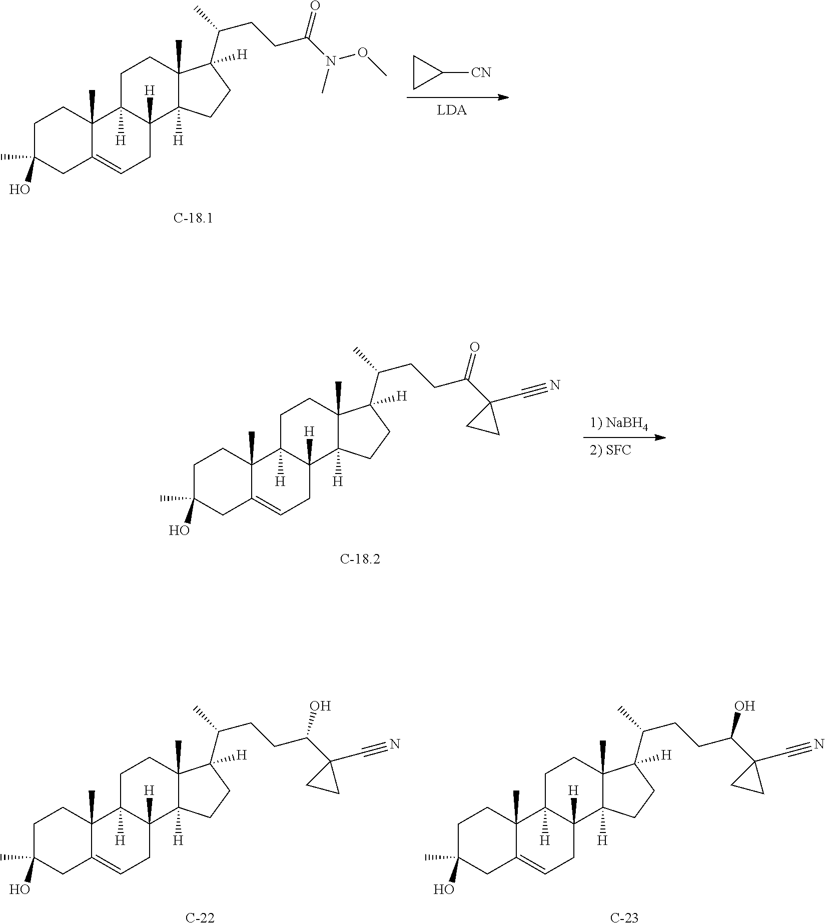

### Synthesis of C-18.2

To a solution of i-Pr₂NH (0.804 mL, 5.75 mmol) in THF (10 mL) under N₂ was added n-BuLi (2.3 mL, 2.5 M, 5.75 mmol) at −60° C. and the resulting mixture was stirred at 60° C. for 10 min and 0° C. for 10 min. Cyclopropanecarbonitrile (385 mg, 5.75 mmol) was added to freshly prepared LDA (5.75 mmol, in 10 ml THF) at −60° C. under N₂ and the mixture was stirred at −60° C. under N₂ for 1 h. A solution of C-18.1 (500 mg, 1.15 mmol; preparation described in WO2018/075699A1) in THF (5 mL) was added to the mixture at −60° C. under N₂ and the mixture was warmed to room temperature. NH₄Cl (sat. aq., 50 mL) was added and the aqueous phase was extracted with DCM (2×50 mL). The combined organic layers were washed with brine (2×50 mL), dried over anhydrous Na₂SO₄, filtered, and concentrated. The residue was purified by silica gel chromatography (0-40% EtOAc in PE) to give C-18.2 (250 mg). ¹H NMR (400 MHz, CDCl₃) δ_{H} 5.33-5.25 (m, 1H), 3.02-2.72 (m, 2H), 2.44-2.40 (m, 1H), 2.09-1.91 (m, 3H), 1.90-1.58 (m, 10H), 1.55-1.13 (m, 12H), 1.11 (s, 3H), 1.10-1.04 (m, 1H), 1.01 (s, 3H), 0.96 (d, J=6.8 Hz, 3H), 0.68 (s, 3H).

### Synthesis of C-22 and C-23

To a solution of C-18.2 (230 mg, 0.53 mmol) in THF (2 mL) and MeOH (2 ml) was added NaBH₄ (19.9 mg, 0.53 mmol) at 25° C. and the resulting mixture was stirred at 25° C. for 10 min. The mixture was poured into saturated aqueous NH₄Cl (10 mL), and the aqueous layer was extracted with DCM (3×10 mL). The combined organic layers were washed with brine (2×20 mL), dried over anhydrous Na₂SO₄, filtered, concentrated, purified by SFC DAICEL CHIRALPAK IG (250 mm*30 mm, 10 um); condition: 0.1% NH₃H₂O ETOH; gradient: 100% B Hold Time (min), Flow Rate (ml/min): 80) and lyophilized to give C-22 (41.4 mg, 61.1%) and C-23 (66.9 mg, 75.3%).

C-22: ¹H NMR (400 MHz, CDCl₃) δ_{H} 5.38-5.27 (m, 1H), 3.08-2.95 (m, 1H), 2.44 (d, J=12.4 Hz, 1H), 2.06-1.62 (m, 10H), 1.56-1.35 (m, 7H), 1.35-1.17 (m, 9H), 1.14 (s, 3H), 1.13-1.05 (m, 2H), 1.04 (s, 3H), 1.00 (d, J=6.4 Hz, 3H), 0.98-0.93 (m, 2H), 0.71 (s, 3H). LC-ELSD/MS 30-90AB_2 min_E, purity>98%, MS ESI calcd. for C₂₉H₄₄NO [M−H₂O+H]⁺ 422.3, found 422.3.

C-23: ¹H NMR (400 MHz, CDCl₃) δ_{H} 5.37-5.24 (m, 1H), 3.15-2.95 (m, 1H), 2.42 (d, J=12.4 Hz, 1H), 2.06-1.57 (m, 11H), 1.53-1.14 (m, 14H), 1.11 (s, 3H), 1.10-1.03 (m, 2H), 1.01 (s, 3H), 0.96 (d, J=6.4 Hz, 3H), 0.94-0.90 (m, 1H), 0.69 (s, 3H). LC-ELSD/MS 30-90AB_2 min_E, purity>98%, MS ESI calcd. for C₂₉H₄₄NO [M−H₂O+H]⁺ 422.3, found 422.3.

### Example 43:1-((1R,4R)-1-hydroxy-4-((3S,8S,9S,10R,13R,14S,17R)-3-hydroxy-3,10,13-trimethyl-2,3,4,7,8,9,10,11,12,13,14,15,16,17-tetradecahydro-1H-cyclopenta[a]phenanthren-17-yl)pentyl)cyclobutane-1-carbonitrile (C-24) and 1-((1S,4R)-1-hydroxy-4-((3S,8S,9S,10R,13R,14S,17R)-3-hydroxy-3,10,13-trimethyl-2,3,4,7,8,9,10,11,12,13,14,15,16,17-tetradecahydro-1H-cyclopenta[a]phenanthren-17-yl)pentyl)cyclobutane-1-carbonitrile (C-25)

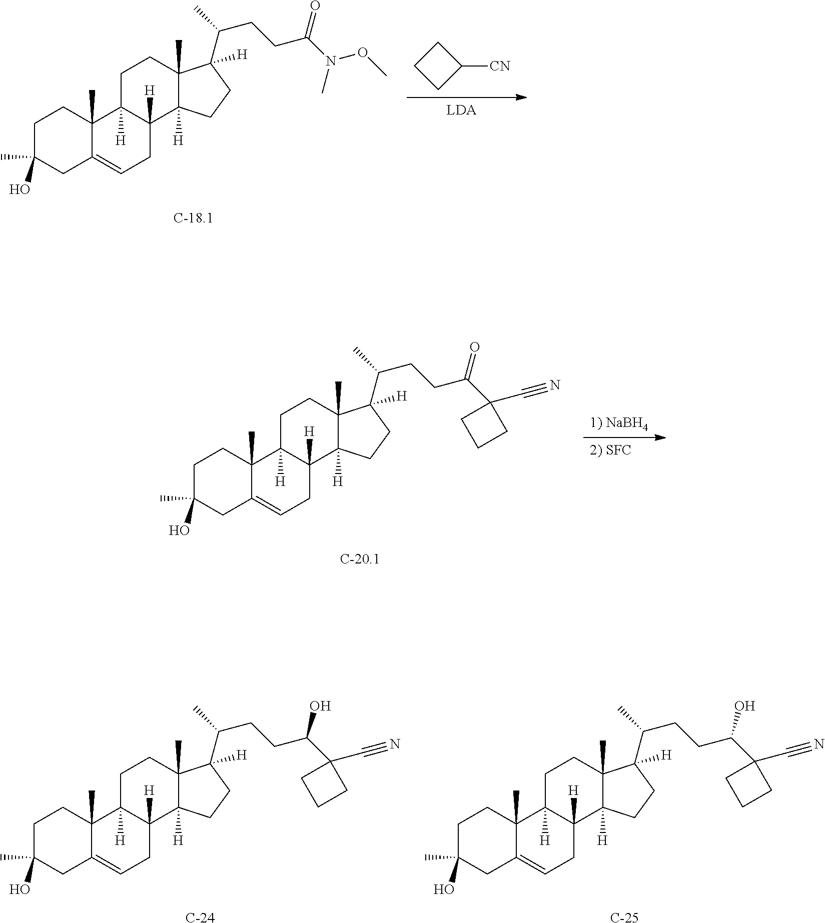

### Synthesis of C-20.1

To a solution of i-Pr₂NH (0.804 mL, 5.75 mmol) in THF (10 mL) under N₂ was added n-BuLi (2.3 mL, 2.5 M, 5.75 mmol) at −60° C. and the resulting mixture was stirred at 60° C. for 10 min and 0° C. for 10 min. Cyclobutanecarbonitrile (466 mg, 5.75 mmol) was added to freshly prepared LDA (5.75 mmol, in 10 ml THF) at −60° C. under N₂ and the mixture was stirred at −60° C. under N₂ for 1 h. A solution of C-18.1 (500 mg, 1.15 mmol) in THF (5 mL) was added to the mixture at −60° C. under N₂ and the mixture was warmed to 20° C. and stirred for 40 min. NH₄Cl (sat. aq., 50 mL) was added and the aqueous phase was extracted with DCM (2×50 mL). The combined organic layers were washed with brine (2×100 mL), dried over anhydrous Na₂SO₄, filtered, and concentrated. The residue was purified by silica gel chromatography (0-40% EtOAc in PE) to give C-20.1 (320 mg). C-20.1 (17 mg, 0.0349 mmol) was lyophilized to give C-20.1 (8.3 mg, 44.2%). ¹H NMR (400 MHz, CDCl₃) δ_{H}=5.32-5.28 (m, 1H), 2.80-2.20 (m, 8H), 2.03-1.59 (m, 9H), 1.55-1.14 (m, 13H), 1.11 (s, 3H), 1.10-1.02 (m, 2H), 1.01 (s, 3H), 0.93 (d, J=6.4 Hz, 3H), 0.68 (s, 3H). LC-ELSD/MS 30-90AB_2 min_E, purity>98%, MS ESI calcd. for C₃₀H₄₄NO [M−H₂O+H]⁺ 434.3, found 434.3.

### Synthesis of C-24 and C-25

To a solution of C-20.1 (300 mg, 0.66 mmol) in THF (2 mL) and MeOH (2 ml) was added NaBH₄ (25.2 mg, 0.66 mmol) at 25° C. and the resulting mixture was stirred for 10 min. The mixture was poured into saturated aqueous NH₄Cl (10 mL), and the aqueous layer was extracted with DCM (3×10 mL). The combined organic layers were washed with brine (2×20 mL), dried over anhydrous Na₂SO₄, filtered, concentrated and purified by SFC (DAICEL CHIRALPAK AD (250 mm*30 mm, 10 um); condition: 0.1% NH₃H₂O ETOH; gradient: from 30% to 30% of B, Flow Rate (ml/min): 21) to give C-24 (90 mg) and C-25 (106 mg). C-24 and C-25 were independently lyophilized to give C-24 (74.7 mg, 83.1%) and C-25 (73.7 mg, 69.5%), respectively.

C-24: ¹H NMR (400 MHz, CDCl₃) δ_{H} 5.34-5.28 (m, 1H), 3.68-3.44 (m, 1H), 2.52-1.94 (m, 10H), 1.91-1.58 (m, 5H), 1.53-1.22 (m, 11H), 1.22-1.13 (m, 2H), 1.12 (s, 3H), 1.10-1.02 (m, 2H), 1.01 (s, 3H), 1.00-0.96 (m, 1H), 0.95 (d, J=6.4 Hz, 3H), 0.69 (s, 3H). LC-ELSD/MS 30-90AB_2 min_E, purity>98%, MS ESI calcd. for C₃₀H₄₆NO [M−H₂O+H]⁺ 436.3, found 436.3.

C-25: ¹H NMR (400 MHz, CDCl₃) δ_{H} 5.33-5.29 (m, 1H), 3.62-3.52 (m, 1H), 2.55-1.89 (m, 12H), 1.87-1.63 (m, 8H), 1.56-1.29 (m, 11H), 1.24-1.16 (m, 2H), 1.11 (s, 3H), 1.01 (s, 3H), 0.95 (d, J=6.8 Hz, 3H), 0.68 (s, 3H). LC-ELSD/MS 30-90AB_2 min_E, purity>98%, MS ESI calcd. for C₃₀H₄₆NO [M−H₂O+H]⁺ 436.3, found 436.3.

### Example 44: Synthesis of 1-((1S,4R)-4-((3S,5S,8S,10S,13R,14S,17R)-3-ethyl-3-hydroxy-10,13-dimethyl-2,3,4,5,6,7,8,10,12,13,14,15,16,17-tetradecahydro-1H-cyclopenta[a]phenanthren-17-yl)-1-hydroxypentyl)cyclopropane-1-carbonitrile (C-26) and 1-((1R,4R)-4-((3S,5S,8S,10S,13R,14S,17R)-3-ethyl-3-hydroxy-10,13-dimethyl-2,3,4,5,6,7,8,10,12,13,14,15,16,17-tetradecahydro-1H-cyclopenta[a]phenanthren-17-yl)-1-hydroxypentyl)cyclopropane-1-carbonitrile (C-27)

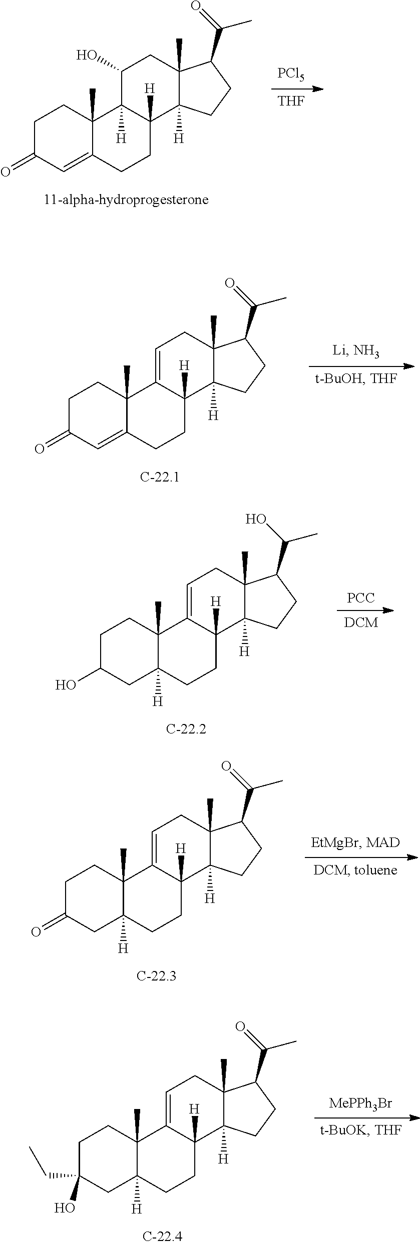

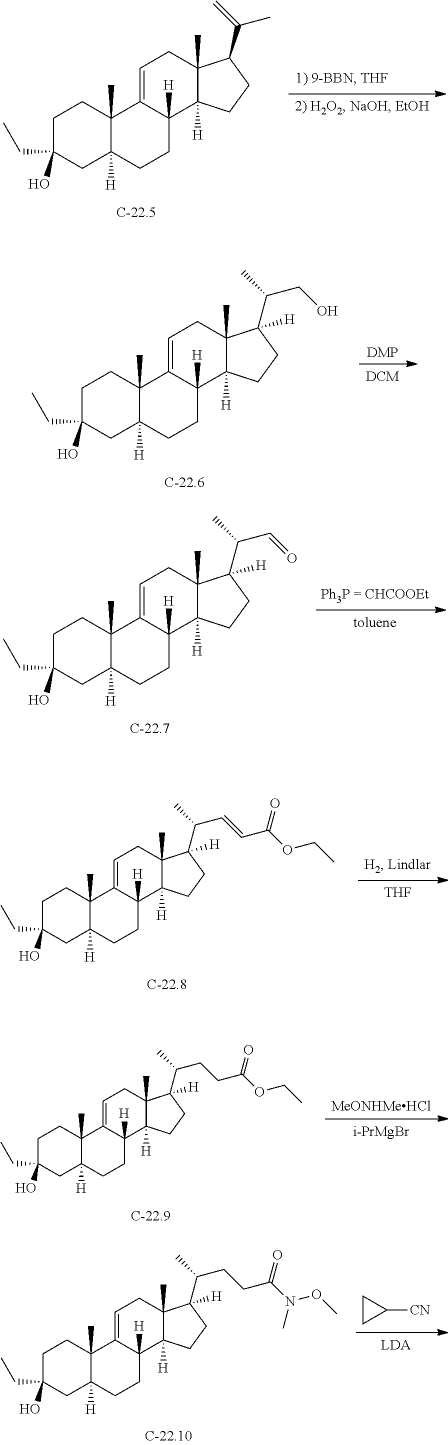

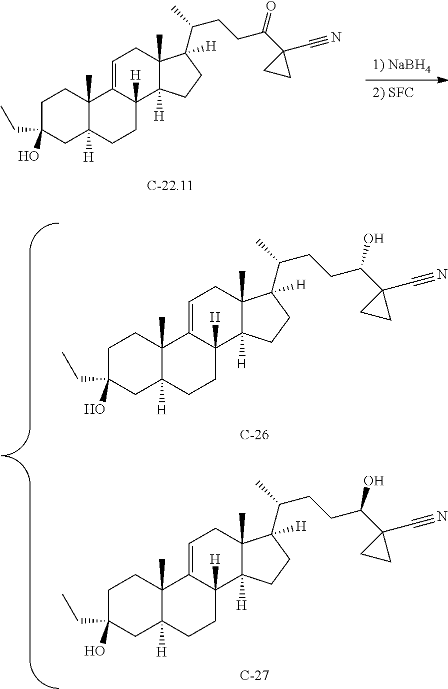

### Synthesis of C-22.1

To a solution of 11-alpha-hydroxyprogesterone (50 g, 151 mmol) in THF (1.5 L) was added PCl₅ (62.8 g, 302 mmol) at −70° C. under N₂ and the resulting mixture was stirred for 1 h. The mixture was quenched with NaHCO₃ (1.5 L, sat. aq.) and extracted with EtOAc (1.5 L). The organic layer was separated, dried over anhydrous Na₂SO₄, filtered, and concentrated to give C-22.1 (60 g).

### Synthesis of C-22.2

Lithium (5.2 g, 750 mmol) was added to liquid ammonia (750 mL) in portions at −70° C. and the resulting mixture was stirred for 1 h. A solution of C-22.1 (47 g, 150 mmol) in dry THF (750 mL) and t-butanol (44.4 g) was added, and the temperature was maintained below-60° C. The resulting mixture was stirred at −70° C. for 1 h. Ammonium chloride (53.4 g) was added to reaction mixture and the mixture was warmed to room temperature and stirred for 16 h. H₂O (500 mL) was added, and the reaction mixture was washed with THF (500 mL). The mixture was filtered and extracted with DCM (500 ml). The mixture was concentrated under vacuum to give C-22.2 (54 g).

### Synthesis of C-22.3

To a solution of C-22.2 (54 g, 169 mmol) in DCM (750 mL) was added silica gel (54.5 g) and PCC (54.5 g, 253 mmol) at 0° C. and the resulting mixture was stirred at 25° C. for 1 h. Petroleum ether (400 mL) was added to the reaction mixture and it was filtered, the filter cake was washed with DCM (2×100 mL), and the filtrate was concentrated under vacuum. The residue was purified by silica gel chromatography (15˜35% EtOAc in PE) to give C-22.3 (31 g, 58.3%). ¹H NMR (400 MHz, CDCl₃) δ_{H} 5.43-5.35 (m, 1H), 2.64-2.17 (m, 8H), 2.10-1.59 (m, 9H), 1.51-1.26 (m, 5H), 1.15 (s, 3H), 1.06-0.94 (m, 1H), 0.58 (s, 3H).

### Synthesis of C-22.4

To a solution of BHT (130 g, 590 mmol) in toluene (260 mL) under nitrogen at 0° C. was added AlMe₃ (2 M in toluene, 147 mL, 295 mmol) dropwise and the resulting mixture was stirred at 25° C. for 1 h. C-22.3 (31 g, 98.5 mmol) in anhydrous DCM (150 mL) was added dropwise at −70° C. and the mixture was stirred at −70° C. for 1 h under N₂. EtMgBr (98.3 mL, 295 mmol, 3M in ethyl ether) was added dropwise at -70° C. and the resulting solution was stirred for 3 h. The reaction mixture was poured into citric acid (600 mL, 20% aq.) at 10° C. and extracted with EtOAc (2×500 mL). The combined organic layers were dried over anhydrous Na₂SO₄, filtered, and concentrated. The residue was purified by a silica gel chromatography (PE/EtOAc=3/1) to give C-22.4 (28 g, 82.5%).

### Synthesis of C-22.5

To a mixture of MePPh₃Br (27.9 g, 78.3 mmol) in THF (300 mL) was added t-BuOK (8.78 g, 78.3 mmol) at 25° C. under N₂ and the resulting mixture was stirred at 45° C. for 30 min. C-22.4 (18 g, 52.2 mmol) was added in portions to keep the internal temperature below 55° C. and the mixture was stirred for 1.5 h. The reaction mixture was quenched with 10% aqueous NH₄Cl (500 mL) at 25° C. and the aqueous layer was extracted with EtOAc (2×500 mL). The combined organic layers were separated, dried over anhydrous Na₂SO₄, filtered, concentrated, triturated with MeOH/H₂O (1:1, 600 mL) and purified by silica gel chromatography (0˜25% of EtOAc in PE) to give C-22.5 (12.6 g, 70.7%). ¹H NMR (400 MHz, CDCl₃) δ_{H} 5.31-5.24 (m, 1H), 4.84 (s, 1H), 4.71 (s, 1H), 2.00-1.65 (m, 11H), 1.48-1.13 (m, 13H), 1.03-0.81 (m, 8H), 0.50 (s, 3H).

### Synthesis of C-22.6

To a solution of C-22.5 (1 g, 2.91 mmol) in THF (5 mL) was added 9-BBN (29 mL, 0.5 M, 14.5 mmol) at 0° C. under N₂ and the resulting mixture was stirred at 25° C. for 16 h. Ethanol (3.43 ml, 58.2 mmol), NaOH (8.72 mL, 5M aq., 43.6 mmol), and H₂O₂ (4.36 mL, 10 M, 43.6 mmol) were added dropwise. After stirring at 25° C. for 1 h, the mixture was quenched with Na₂SO₃ (10 mL, sat. aq.) and the mixture was concentrated to remove most of the organic solvent and filtered. The solid was washed with water (10 mL), dried in vacuum and purified by silica gel chromatography (0-15% EA in PE) to give C-22.6 (600 mg, 57.6%). ¹H NMR (400 MHz, CDCl₃) δ_{H} 5.28-5.21 (m, 1H), 3.68-3.59 (m, 1H), 3.42-3.35 (m, 1H), 2.20-2.11 (m, 1H), 2.03-1.64 (m, 8H), 1.43-1.15 (m, 14H), 1.06-0.84 (m, 11H), 0.61 (s, 3H).

### Synthesis of C-22.7

To a solution of C-22.6 (2 g, 5.54 mmol) in DCM (20 mL) was added DMP (3.51 g, 8.30 mmol) at 25° C. and the resulting mixture was stirred for 5 min. NaHCO₃ (40 mL, sat. aq.) and Na₂S₂O₃ (40 mL, sat. aq.) were added and the aqueous phase was extracted with DCM (2×20 mL). The combined organic layers were washed with NaHCO₃/Na₂S₂O₃ (1:1, 2×40 mL, sat. aq.), brine (40 mL), dried over anhydrous Na₂SO₄, filtered, and concentrated. The mixture was purified by silica gel chromatography (15˜30% of EtOAc in PE) to give C-22.7 (1.6 g, 80%). ¹H NMR (400 MHz, CDCl₃) δ_{H} 9.59-9.58 (m, 1H), 5.28-5.23 (m, 1H), 2.39-2.34 (m, 1H), 2.17-2.09 (m, 1H), 2.01-1.81 (m, 5H), 1.45-1.39 (m, 7H), 1.30-1.21 (m, 6H), 1.14-1.11 (m, 3H), 0.97 (s, 3H), 0.90-0.85 (m, 7H), 0.64 (s, 3H).

### Synthesis of C-22.8

To a solution of C-22.7 (1.6 g, 4.46 mmol) in toluene (15 mL) Ph₃P=CHCOOEt (3.1 g, 8.92 mmol) was added and the resulting mixture was stirred at 110° C. for 2 h. The mixture was poured into saturated aqueous NH₄Cl (20 mL) and extracted with EtOAc (2×20 mL). The combined organic layers were washed with brine (20 mL), dried over anhydrous Na₂SO₄, filtered, and concentrated in vacuo. The residue was purified by silica gel chromatography (15-20% EA in PE) to give C-22.8 (1.2 g, 62.8%). C-22.8 (0.9 g) was further purified by SFC (Column: DAICEL CHIRALPAK AD (250 mm*30 mm, 10 um); Condition: 0.1% NH₃H₂O ETOH; Begin B: 25%; End B: 25%; FlowRate (ml/min): 60; Injections: 70) to give C-22.8 (0.62 g, 69.2%). ¹H NMR (400 MHz, CDCl₃) δ_{H} 6.91-6.74 (m, 1H), 5.72 (d, J=15.6 Hz, 1H), 5.28-5.21 (m, 1H), 4.17 (q, J=7.2 Hz, 2H), 2.34-1.83 (m, 5H), 1.75-1.53 (m, 9H), 1.44-1.20 (m, 13H), 1.08 (d, J=6.4 Hz, 3H), 0.96 (s, 3H), 0.90-0.84 (m, 3H), 0.63 (s, 3H).

### Synthesis of C-22.9

To a solution of C-22.8 (0.62 g, 1.44 mmol) in THF (20 mL) was added Lindlar catalyst (400 mg) and the resulting mixture was stirred at 25° C. under H₂ for 18 h. The mixture was filtered and concentrated to give C-22.9 (0.6 g, 96.7%). C-22.9 (20 mg) was lyophilized to give C-22.9 (8.2 mg, 41.2%).

### Synthesis of C-22.10

To a suspension of C-22.9 (2.4 g, 5.57 mmol) and MeNHOMe·HCl (814 mg, 8.35 mmol) in THF (30 mL) was added i-PrMgBr (2 M in THF, 11.1 mL, 22.2 mmol) at 0° C. under N₂ and the resulting mixture was stirred at 20° C. for 30 min. NH₄Cl (10% aq., 50 mL) was added and the aqueous layer was extracted with DCM (2×50 mL). The combined organic layers were washed with brine (50 mL), dried over anhydrous Na₂SO₄, filtered, and concentrated. The residue was purified by silica gel chromatography (0%˜15% of EtOAc in PE) to give C-22.10 (1.96 g, 79%). ¹H NMR (400 MHz, CDCl₃) δ_{H} 5.27-5.23 (m, 1H), 3.69 (s, 3H), 3.17 (s, 3H), 2.53-2.26 (m, 2H), 2.21-2.07 (m, 1H), 2.02-1.73 (m, 6H), 1.63-1.53 (m, 7H), 1.44-1.15 (m, 12H), 0.96 (s, 3H), 0.94 (d, J=6.4 Hz, 3H), 0.87 (t, J=7.6 Hz, 3H), 0.59 (s, 3H).

### Synthesis of C-22.11

To a solution of i-Pr₂NH (0.937 mL, 6.7 mmol) in THF (10 mL) under N₂ was added n-BuLi (2.68 mL, 2.5 M, 6.7 mmol) at −60° C. and the resulting mixture was stirred at 60° C. for 10 min and 0° C. for 10 min. Cyclopropanecarbonitrile (717 mg, 10.7 mmol) was added to the freshly prepared LDA (6.7 mmol, in 6 ml THF) at −60° C. under N₂ and the mixture was stirred at −60° C. under N₂ for 1 h. A solution of C-22.10 (600 mg, 1.34 mmol) in THF (7 mL) was added to the mixture at −60° C. under N₂ and the mixture was warmed to 25° C. and stirred for 30 min. NH₄Cl (sat. aq., 50 mL) was added and the mixture was extracted with EtOAc, washed with brine (2×30 mL), dried over anhydrous Na₂SO₄, filtered, and concentrated. The residue was purified by silica gel chromatography (0˜20% EtOAc in PE) to give C-22.11 (410 mg, 67.7%). C-22.11 (20 mg, 0.0443 mmol) was lyophilized to give C-22.11 (15.3 mg, 76.8%). ¹H NMR (400 MHz, CDCl₃) δ_{H} 5.26-5.22 (m, 1H), 3.05-2.74 (m, 2H), 2.22-2.08 (m, 1H), 2.03-1.59 (m, 13H), 1.56-1.28 (m, 10H), 1.27-1.07 (m, 6H), 0.97-0.94 (m, 6H), 0.87 (t, J=7.6 Hz, 3H), 0.59 (s, 3H). LC-ELSD/MS 30-90AB_2 min_E, purity>98%, MS ESI calcd. for C₃₀H₄₆NO [M−H₂O+H]⁺ 434.4, found 434.4.

### Synthesis of C-26 and C-27

To a solution of C-22.11 (380 mg, 0.8412 mmol) in THF (3 mL) and MeOH (3 ml) was added NaBH₄ (80 mg, 2.1 mmol) at 25° C. and the resulting mixture was stirred for 10 min. Saturated aqueous NH₄Cl (10 mL) was added, and the aqueous layer was extracted with DCM (3×10 mL). The combined organic layers were washed with brine (2×10 mL), dried over anhydrous Na₂SO₄, filtered, and concentrated. The residue was purified by silica gel chromatography (0˜30% of EtOAc in PE) and further purified by SFC (DAICEL CHIRALPAK AD (250 mm*30 mm, 10 um); condition: 0.1% NH₃H₂O MEOH; gradient: from 35% to 35% of B, Flow Rate (ml/min): 70) to give C-26 (150 mg) and C-27 (140 mg). C-26 and C-27 were independently lyophilized to give C-26 (112.7 mg, 75.1%) and C-27 (124.5 mg, 89.2%), respectively.

C-26: ¹H NMR (400 MHz, CDCl₃) δ_{H} 5.26-5.22 (m, 1H), 3.07-2.91 (m, 1H), 2.24-2.09 (m, 1H), 2.01-1.58 (m, 13H), 1.55-1.28 (m, 8H), 1.28-0.98 (m, 10H), 0.98-0.95 (m, 6H), 0.94-0.90 (m, 1H), 0.88 (t, J=7.6 Hz, 3H), 0.60 (s, 3H). LC-ELSD/MS 30-90AB_2 min_E, purity>98%, MS ESI calcd. for C₃₀H₄₆NO [M−H₂O+H]⁺ 436.3, found 436.3.

C-27: ¹H NMR (400 MHz, CDCl₃) δ_{H} 5.26-5.22 (m, 1H), 3.15-2.89 (m, 1H), 2.22-2.09 (m, 1H), 2.02-1.59 (m, 11H), 1.56-1.36 (m, 7H), 1.36-0.98 (m, 12H), 0.96 (s, 3H), 0.95 (d, J=7.2 Hz, 3H), 0.93-0.90 (m, 1H), 0.87 (t, J=7.2 Hz, 3H), 0.60 (s, 3H). LC-ELSD/MS 30-90AB_2 min_E, purity>98%, MS ESI calcd. for C₃₀H₄₆NO [M−H₂O+H]⁺ 436.3, found 436.3.

### Example 45: (3R,6R)-6-((3S,5S,8S,10S,13R,14S,17R)-3-ethyl-3-hydroxy-10,13-dimethyl-2,3,4,5,6,7,8,10,12,13,14,15,16,17-tetradecahydro-1H-cyclopenta[a]phenanthren-17-yl)-3-hydroxy-2,2-dimethylheptanenitrile (C-28) and (3S,6R)-6-((3S,5S,8S,10S,13R,14S,17R)-3-ethyl-3-hydroxy-10,13-dimethyl-2,3,4,5,6,7,8,10,12,13,14,15,16,17-tetradecahydro-1H-cyclopenta[a]phenanthren-17-yl)-3-hydroxy-2,2-dimethylheptanenitrile (C-29)

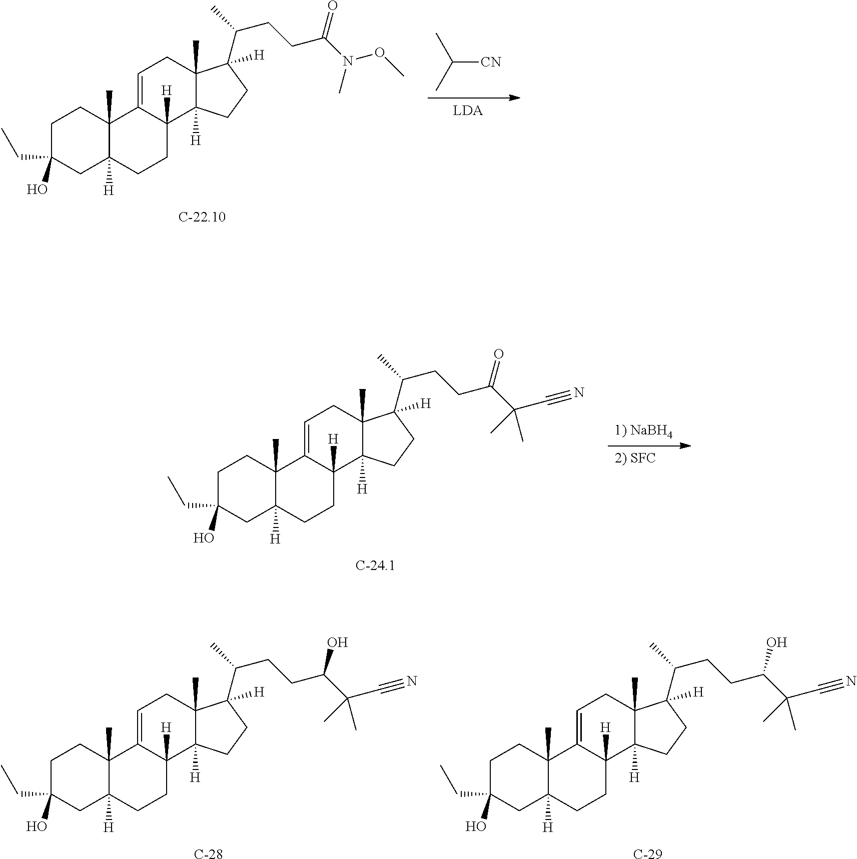

### Synthesis of C-24.1

To a solution of i-Pr₂NH (0.937 mL, 6.7 mmol) in THF (6 mL) under N₂ was added n-BuLi (2.68 mL, 2.5 M, 6.7 mmol) at −60° C. and the resulting mixture was stirred at −60° C. for 10 min and 0° C. for 10 min. 2-methylpropanenitrile (820 mg, 11.8 mmol) was added to the freshly prepared LDA (6.7 mmol, in 6 ml THF) at −60° C. under N₂ and the mixture was stirred at −60° C. under N₂ for 1 h. A solution of C-22.10 (600 mg, 1.34 mmol) in THF (6 mL) was added to the mixture at −60° C. under N₂ and the mixture was warmed to 25° C. and stirred for 30 min. NH₄Cl (sat. aq., 50 mL) was added and the mixture was extracted with EtOAc, washed with brine (2×30 mL), dried over anhydrous Na₂SO₄, filtered, and concentrated. The residue was purified by silica gel chromatography (0˜30% EtOAc in PE) to give C-24.1 (390 mg). C-24.1 (40 mg) was lyophilized to give C-24.1 (18.9 mg, 47.3%). ¹H NMR (400 MHz, CDCl₃) δ_{H} 5.23 (d, J=6.0 Hz, 1H), 2.85-2.68 (m, 2H), 2.19-1.58 (m, 12H), 1.50 (s, 6H), 1.45-1.12 (m, 14H), 0.96 (s, 3H), 0.93 (d, J=6.4 Hz, 3H), 0.87 (t, J=7.6 Hz, 3H), 0.59 (s, 3H).

### Synthesis of C-28 and C-29

To a solution of C-24.1 (350 mg, 0.771 mmol) in THF (3 mL) and MeOH (3 ml) was added NaBH₄ (80 mg, 2.1 mmol) at 25° C. and the resulting mixture was stirred for 10 min. Saturated aqueous NH₄Cl (10 mL) was added and the aqueous layer was extracted with DCM (3×10 mL). The combined organic layers were washed with brine (2×10 mL), dried over anhydrous Na₂SO₄, filtered, and concentrated. The residue was purified by silica gel chromatography (0˜30% of EtOAc in PE) to give C-28 and C-29 (257 mg). C-28 and C-29 (257 mg) were further purified by SFC (DAICEL CHIRALPAK AD (250 mm*30 mm, 10 um); condition: 0.1% NH₃H₂O MEOH; gradient: from 30% to 30% of B, Flow Rate (ml/min): 70) to give C-28 (90 mg) and C-29 (60 mg). C-28 (90 mg) and C-29 (60 mg) were independently lyophilized to give C-28 (67.5 mg, 19.2%) and C-29 (53 mg, 15.8%), respectively.

C-28: ¹H NMR (400 MHz, CDCl₃) δ_{H} 5.26-5.22 (m, 1H), 3.44-3.36 (m, 1H), 2.24-2.09 (m, 1H), 2.03-1.57 (m, 11H), 1.55-1.39 (m, 7H), 1.38 (s, 3H), 1.34 (s, 3H), 1.33-1.08 (m, 8H), 0.97 (s, 3H), 0.94 (d, J=6.0 Hz, 3H), 0.88 (t, J=7.6 Hz, 3H), 0.60 (s, 3H). LC-ELSD/MS 30-90AB_2 min_E, purity>98%, MS ESI calcd. for C₃₀H₄₈NO [M−H₂O+H]⁺ 438.4, found 438.4.

C-29: ¹H NMR (400 MHz, CDCl₃) δ_{H} 5.27-5.21 (m, 1H), 3.70-3.03 (m, 1H), 2.25-2.09 (m, 1H), 2.03-1.57 (m, 12H), 1.54-1.39 (m, 5H), 1.38 (s, 3H), 1.34 (s, 3H), 1.33-0.98 (m, 9H), 0.97 (s, 3H), 0.95 (d, J=6.4 Hz, 3H), 0.88 (t, J=7.6 Hz, 3H), 0.60 (s, 3H). LC-ELSD/MS 30-90AB_2 min_E, purity>98%, MS ESI calcd. for C₃₀H₄₈NO [M−H₂O+H]⁺ 438.4, found 438.4.

### Example 46: Synthesis of 1-((1S,4R)-1-hydroxy-4-((3S,5S,8S,10S,13R,14S,17R)-3-hydroxy-3,10,13-trimethyl-2,3,4,5,6,7,8,10,12,13,14,15,16,17-tetradecahydro-1H-cyclopenta[a]phenanthren-17-yl)pentyl)cyclobutane-1-carbonitrile (C-30) and 1-((1R,4R)-1-hydroxy-4-((3S,5S,8S,10S,13R,14S,17R)-3-hydroxy-3,10,13-trimethyl-2,3,4,5,6,7,8,10,12,13,14,15,16,17-tetradecahydro-1H-cyclopenta[a]phenanthren-17-yl)pentyl)cyclobutane-1-carbonitrile (C-31)

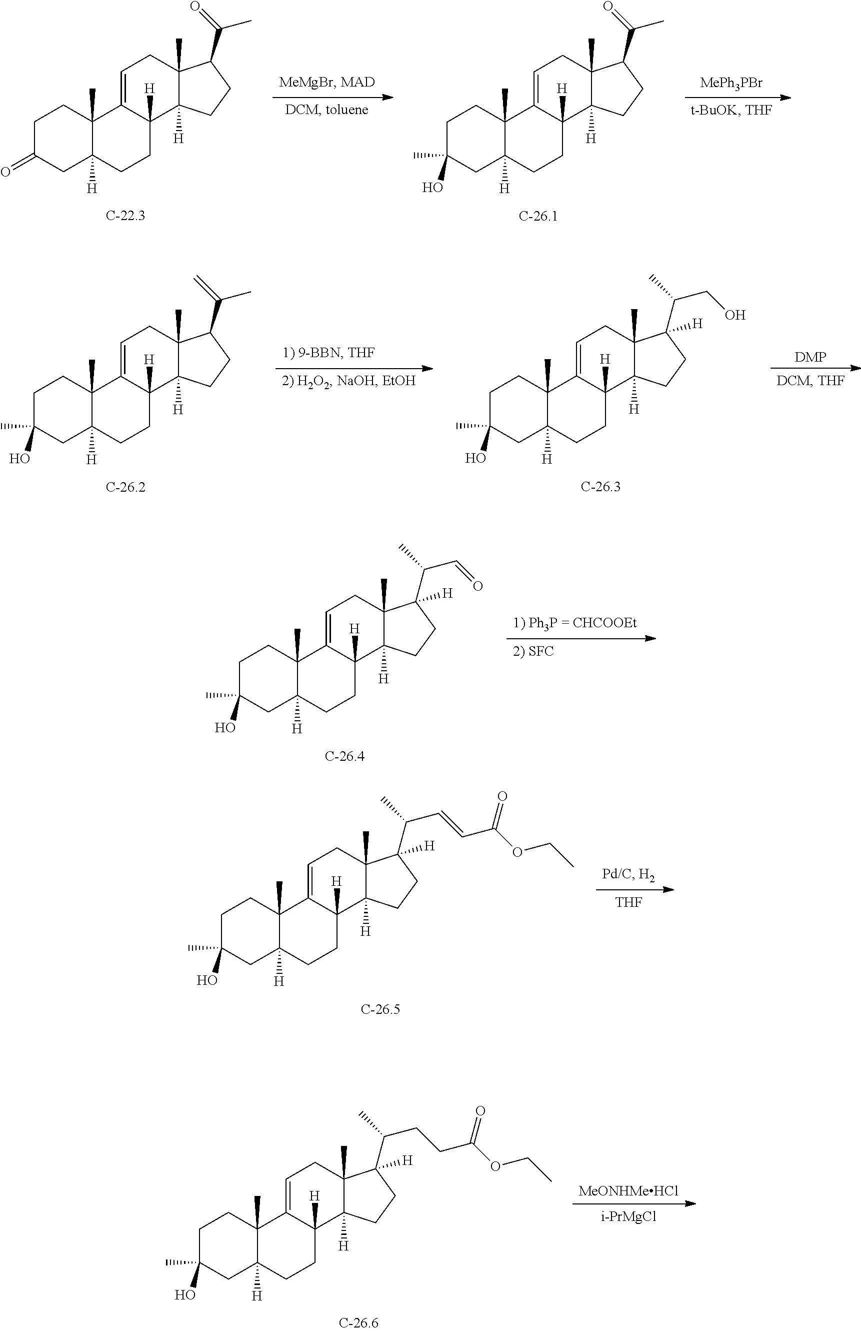

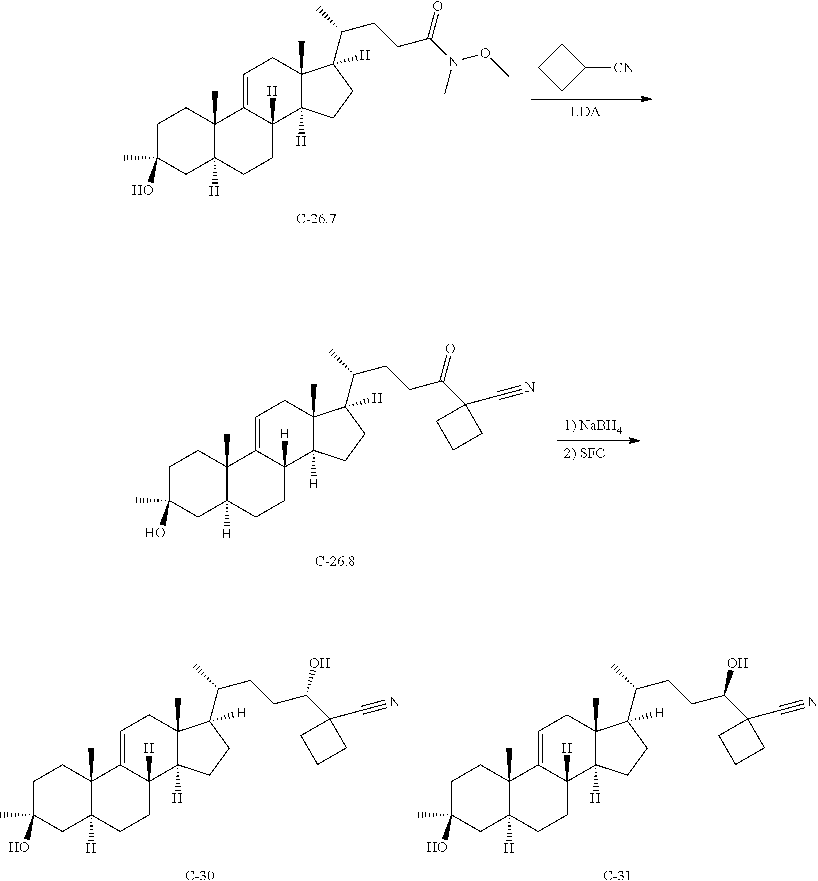

### Synthesis of C-26.1

To a solution of 2,6-di-tert-butyl-4-methylphenol (40 g, 181 mmol) in toluene (400 mL) was added dropwise AlMe₃ (45.2 mL, 90.5 mmol, 2 M in toluene) at 0° C. under N₂ and the resulting mixture was stirred at 25° C. for 1 h. To the freshly prepared solution of MAD (48.5 g, 101 mmol) under N₂ at -70° C. was added a solution of C-22.3 (8 g, 25.4 mmol) in anhydrous DCM (200 mL) dropwise. The mixture was stirred at −70° C. for 1 h, and methylmagnesium bromide (3 M in diethyl ether, 33.6 mL, 101 mmol) was added dropwise. After stirring at −70° C. for 2 h, the reaction mixture was quenched with aqueous citric acid (200 mL), extracted with EtOAc (100 mL) three times, dried over anhydrous sodium sulfate, filtered, concentrated, and purified by silica gel chromatography (0˜45% of EtOAc in PE) to give C-26.1 (8 g). ¹H NMR (400 MHz, CDCl₃) δ_{H} 5.32 (br s, 1H), 2.58 (t, J=9.2 Hz, 1H), 2.25 (br d, J=3.1 Hz, 3H), 2.13 (s, 3H), 2.06-1.81 (m, 4H), 1.51-1.28 (m, 12H), 1.25 (s, 4H), 0.94 (s, 3H), 0.55 (s, 3H).

### Synthesis of C-26.2

To a mixture of MePPh₃Br (29 g, 81.6 mmol) in THF (500 mL) was added t-BuOK (9.13 g, 81.6 mmol) at 25° C. under N₂ and the resulting mixture was stirred at 50° C. for 30 min. C-26.1 (9 g, 27.2 mmol) was added in portions to keep the internal temperature below 50° C. After stirring at 50° C. for 1 h, the reaction mixture was quenched with 10% aqueous NH₄Cl (300 mL) at 25° C. and the aqueous layer was extracted with EtOAc (400 mL). The combined organic layers were concentrated under vacuum, and the residue was triturated with MeOH/H₂O (200 mL/200 mL) to give C-26.2 (8.2 g). ¹H NMR (400 MHz, CDCl₃) δ_{H} 5.30 (s, 1H), 4.85 (s, 1H), 4.71 (s, 1H), 2.05 (br s, 6H), 1.71-1.38 (m, 10H), 1.25 (s, 10H), 0.95 (s, 4H), 0.56-0.47 (m, 3H).

### Synthesis of C-26.3

To a solution of C-26.3 (4 g, 12.1 mmol) in THF (50 mL) was added 9-BBN dimer (145 mL, 72.6 mmol, 0.5 M) and the resulting mixture was stirred at 20° C. for 16 h. Ethanol (8.55 mL, 145 mmol) was added at 0° C. and the mixture was stirred for 30 min. Aqueous NaOH (28.9 mL, 5.0 M, 145 mmol) was added at 0° C. and H₂O₂ (14.4 mL, 10 M, 145 mmol) was added. After stirring at 60° C. for 1 h, aqueous Na₂SO₃ (100 mL, 15%) was added and the mixture was extracted with EtOAc (3×200 mL). The combined organic layers were washed with brine (200 mL), dried over anhydrous Na₂SO₄, filtered, concentrated, and purified by silica gel chromatography (0˜45% of EtOAc in PE) to give C-26.3 (5.5 g). ¹H NMR (400 MHz, CDCl₃) δ_{H} 5.26 (br d, J=5.6 Hz, 1H), 3.65 (dd, J=3.2, 10.5 Hz, 1H), 3.44-3.34 (m, 1H), 2.20-2.11 (m, 1H), 2.08-1.92 (m, 3H), 1.90-1.69 (m, 5H), 1.44-1.29 (m, 7H), 1.24 (s, 9H), 1.04 (d, J=6.4 Hz, 3H), 0.94 (s, 4H), 0.61 (s, 3H).

### Synthesis of C-26.4

To a solution of C-26.3 (7.65 g, 22.0 mmol) in DCM (200 mL) and THF (30 mL) was added NaHCO₃ (7.75 g, 92.3 mmol) and DMP (23.2 g, 54.9 mmol) at 25° C. and the resulting mixture was stirred for 30 min. NaHCO₃ (300 mL, sat. aq.) and Na₂S₂O₃ (150 mL, sat. aq.) were added and the aqueous phase was extracted with DCM (2×100 mL). The combined organic layers were washed with brine (2×100 mL), dried over anhydrous Na₂SO₄, filtered, and concentrated to give C-26.4 (5.6 g). ¹H NMR (400 MHz, CDCl₃) δ_{H} 9.58 (d, J=3.2 Hz, 1H), 5.28-5.25 (m, 1H), 2.41-2.32 (m, 1H), 2.18-1.66 (m, 11H), 1.59-1.32 (m, 10H), 1.24 (s, 3H), 1.12 (d, J=6.8 Hz, 3H), 0.95 (s, 3H), 0.64 (s, 3H).

### Synthesis of C-26.5

To a solution of C-26.4 (10.3 g, 29.8 mmol) in toluene (150 mL) was added Ph₃P=CHCOOEt (36.2 g, 104 mmol) and the resulting mixture was stirred at 110° C. for 2 h. The mixture was poured into saturated aqueous NH₄Cl (200 mL) and extracted with EtOAc (3×100 mL). The combined organic layers were washed with brine (100 mL), dried over anhydrous Na₂SO₄, filtered, and concentrated in vacuo. The residue was purified by silica gel chromatography (0˜20% EtOAc in PE) and further purified by SFC twice (column: DAICEL CHIRALCEL OJ (250 mm*50 mm, 10 um; condition: 0.1% NH₃H₂O ETOH; gradient: from 20% to 20% of B, FlowRate (ml/min): 200) to give C-26.5 (5.75 g, 46.7%). ¹H NMR (400 MHz, CDCl₃) δ_{H} 6.83 (dd, J=8.8, 15.6 Hz, 1H), 5.74 (d, J=15.6 Hz, 1H), 5.26 (d, J=5.8 Hz, 1H), 4.17 (q, J=7.2 Hz, 2H), 2.32-1.57 (m, 12H), 1.52-1.26 (m, 13H), 1.24 (s, 3H), 1.09 (d, J=6.8 Hz, 3H), 0.95 (s, 3H), 0.63 (s, 3H). LC-ELSD/MS 10-80AB_7 min_E, purity >99%, MS ESI calcd. for C₂₇H₄₃₀₃ [M+H]⁺ 415.4 found 415.4.

### Synthesis of C-26.6

To a solution of C-26.5 (5.70 g, 13.7 mmol) in THF (30 mL) was added Pd/C (wet, 10%, 3 g) under N₂ atmosphere. The mixture was then stirred under H₂ atmosphere (15 psi) at 25° C. for 16 h. The mixture was filtered, and the filter cake was washed with THF (20 mL) and the filtrate was concentrated. The residue was purified by silica gel chromatography (0˜20% EtOAc in PE) to give C-26.6 (5.30 g, 92.9%). ¹H NMR (400 MHz, CDCl₃) δ_{H} 5.25 (d, J=6.0 Hz, 1H), 4.12 (q, J=7.2 Hz, 2H), 2.39-1.72 (m, 11H), 1.71-1.28 (m, 15H), 1.27-1.25 (m, 3H), 1.24 (s, 3H), 0.94 (s, 3H), 0.92 (d, J=6.0 Hz, 3H), 0.59 (s, 3H).

### Synthesis of C-26.7

To a suspension of C-26.6 (1 g, 2.40 mmol) and MeNHOMe·HCl (350 mg, 3.59 mmol) in THF (10 mL) was added i-PrMgCl (2 M in THF, 4.8 mL, 9.60 mmol) at 0° C. under N₂ and the resulting mixture was stirred at 20° C. for 10 min. NH₄Cl (10% aq., 50 mL) was added and the aqueous layer was extracted with DCM (2×50 mL). The combined organic layers were washed with brine (50 mL), dried over anhydrous Na₂SO₄, filtered, and concentrated to give C-26.7 (1 g, 97.0%). ¹H NMR (400 MHz, CDCl₃) δ_{H} 5.25 (d, J=6.0 Hz, 1H), 3.69 (s, 3H), 3.18 (s, 3H), 2.49-2.28 (m, 2H), 2.20-2.10 (m, 1H), 2.06-1.58 (m, 10H), 1.54-1.27 (m, 10H), 1.24-1.09 (m, 5H), 0.96-0.93 (m, 6H), 0.92-0.80 (m, 1H), 0.60 (s, 3H)

### Synthesis of C-26.8

Cyclobutanecarbonitrile (932 mg, 11.5 mmol) was added to freshly prepared LDA (11.5 mmol, 2M, in 5 ml THF) at −60° C. under N₂ and the resulting mixture was stirred under N₂ for 1 h. A solution of C-26.7 (1 g, 2.31 mmol) in THF (5 mL) was added to the mixture at −60° C. under N₂ and the mixture was warmed to room temperature. NH₄Cl (sat. aq., 50 mL) was added and the aqueous phase was extracted with DCM (2×50 mL). The combined organic layers were washed with brine (2×100 mL), dried over anhydrous Na₂SO₄ filtered, and concentrated in vacuo. The residue was purified by silica gel chromatography (0˜40% EtOAc in PE) to give C-26.8 (1.0 g, 96.1%). ¹H NMR (400 MHz, CDCl₃) δ_{H} 5.25 (d, J=5.6 Hz, 1H), 2.72-2.44 (m, 6H), 2.25-1.60 (m, 13H), 1.49-1.29 (m, 10H), 1.24 (s, 3H), 1.22-1.13 (m, 3H), 0.94 (s, 3H), 0.93 (d, J=6.4 Hz, 3H), 0.59 (s, 3H). LC-ELSD/MS 30-90AB_2 min_E, purity 100%, MS ESI calcd. for C₃₀H+NO [M−H₂O+H]⁺ 434.2, found 434.2.

### Synthesis of C-30 and C-31

To a solution of C-26.8 (1 g, 2.16 mmol) in MeOH (10 mL) was added NaBH₄ (122 mg, 3.23 mmol) at 20° C. and the resulting mixture was stirred for 2 h. The residue was poured into NH₄Cl (100 mL, sat. aq.) and the aqueous phase was extracted with EtOAc (3×50 mL). The combined organic layers were washed with brine (2×50 mL), dried over anhydrous Na₂SO₄, filtered, and concentrated. The residue was purified by silica gel chromatography (0˜40% EtOAc in PE) and further purified by SFC (DAICEL CHIRALPAK AD-H (250 mm*30 mm, 5 um)); Condition: 0.1% NH₃H₂O ETOH; Begin B: 35; End B: 35; FlowRate (mL/min): 80; Injections: 120) to give C-30 (320 mg, 32.8%) and C-31 (320 mg, 32.8%).

C-30: ¹H NMR (400 MHz, CDCl₃) δ_{H} 5.26 (d, J=6.0 Hz, 1H), 3.58 (d, J=8.4 Hz, 1H), 2.48-2.35 (m, 3H), 2.05-1.57 (m, 16H), 1.50-1.27 (m, 13H), 1.24 (s, 3H), 1.07-1.00 (m, 1H), 0.97-0.92 (m, 6H), 0.60 (s, 3H). LC-ELSD/MS 30-90AB_2 min_E, purity>99%, MS ESI calcd. for C₃₀H₄₆ NO [M−H₂O+H]⁺ 436.4, found 436.4.

C-31: ¹H NMR (400 MHz, CDCl₃) δ_{H} 5.25 (d, J=5.6 Hz, 1H), 3.62 (s, 1H), 2.49-2.12 (m, 6H), 2.06-1.60 (m, 15H), 1.56-1.34 (m, 12H), 1.24 (s, 3H), 0.96-0.92 (m, 6H), 0.60 (s, 3H). LC-ELSD/MS 30-90AB_2 min_E, purity>99%, MS ESI calcd. for C₃₀H₄₆NO [M−H₂O+H]⁺ 436.4, found 436.4.

### Example 47: Synthesis of 1-((1R,4R)-1-hydroxy-4-((3S,5S,8S,10S,13R,14S,17R)-3-hydroxy-3,10,13-trimethyl-2,3,4,5,6,7,8,10,12,13,14,15,16,17-tetradecahydro-1H-cyclopenta[a]phenanthren-17-yl)pentyl)cyclopropane-1-carbonitrile (C-32) and 1-((1S,4R)-1-hydroxy-4-((3S,5S,8S,10S,13R,14S,17R)-3-hydroxy-3,10,13-trimethyl-2,3,4,5,6,7,8,10,12,13,14,15,16,17-tetradecahydro-1H-cyclopenta[a]phenanthren-17-yl)pentyl)cyclopropane-1-carbonitrile (C-33)

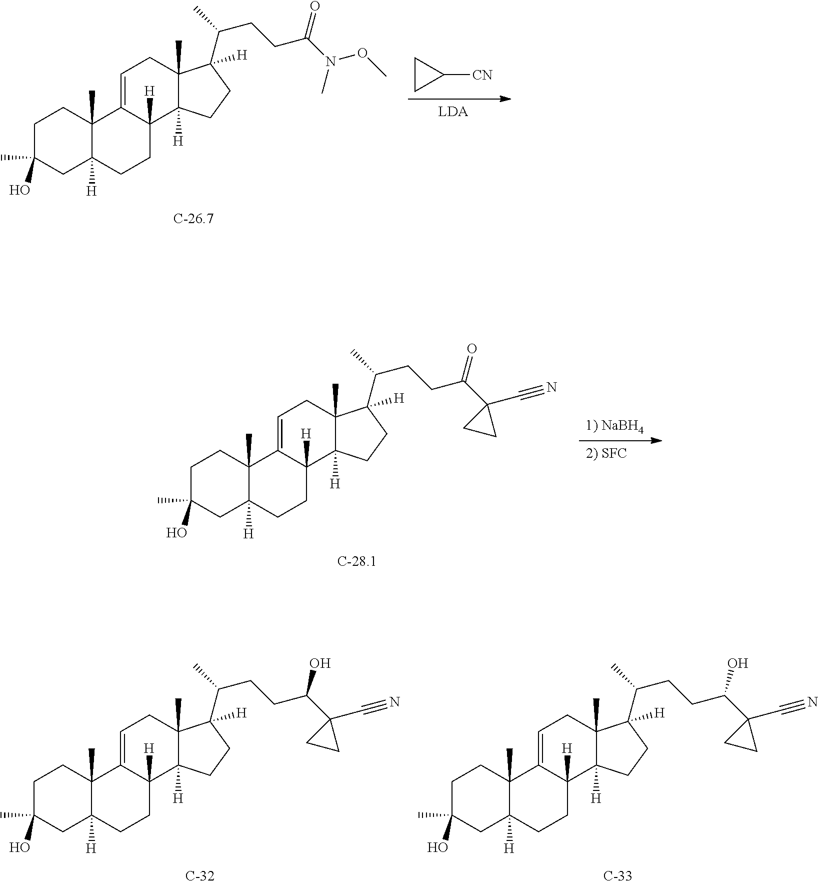

### Synthesis of C-28.1

Cyclopropanecarbonitrile (385 mg, 5.75 mmol) was added to freshly prepared LDA (5.75 mmol, in 5 ml THF) at −60° C. under N₂ and the resulting mixture was stirred under N₂ for 1 h. A solution of C-26.7 (500 mg, 1.15 mmol) in THF (3 mL) was added to the mixture at −60° C. under N₂ and the mixture was warmed to room temperature. NH₄Cl (sat. aq., 30 mL) was added and the aqueous phase was extracted with DCM (2×50 mL). The combined organic layers were washed with brine (2×50 mL), dried over anhydrous Na₂SO₄, filtered, and concentrated in vacuo. The residue was purified by silica gel chromatography (0-40% EtOAc in PE) to give C-28.1 (390 mg, 77.5%). ¹H NMR (400 MHz, CDCl₃) δ_{H} 5.26 (d, J=6.0 Hz, 1H), 3.04-2.80 (m, 2H), 2.20-2.09 (m, 1H), 1.97-1.58 (m, 13H), 1.50-1.29 (m, 11H), 1.24 (s, 3H), 1.20-1.05 (m, 3H), 0.97-0.94 (m, 6H), 0.60 (s, 3H). LC-ELSD/MS 30-90AB_2 min_E, purity>99%, MS ESI calcd. for C₂₉H₄₂NO [M−H₂O+H]⁺ 420.3, found 420.3.

### Synthesis of C-32 and C-33

To a solution of C-28.1 (370 mg, 0.849 mmol) in MeOH (5 mL) was added NaBH₄ (48.2 mg, 1.27 mmol) at 20° C. and the resulting mixture was stirred for 2 h. The mixture was poured into NH₄Cl (30 mL, sat. aq.) and the aqueous phase was extracted with EtOAc (3×50 mL). The combined organic layers were washed with brine (2×50 mL), dried over anhydrous Na₂SO₄, filtered, and concentrated. The residue was purified by silica gel chromatography (0˜40% of EtOAc in PE) and further purified by SFC (DAICEL CHIRALPAK AD (250 mm*30 mm, 10 um)); Condition: 0.1% NH₃H₂O ETOH; Begin B: 30; End B: 30; FlowRate (ml/min): 80; Injections: 120) to give C-32 (183 mg, 47.9%) and C-33 (154 mg, 40.3%). C-32 (183 mg) and C-33 (154 mg) were independently lyophilized to give C-32 (124 mg, 68.1%) and C-33 (27 mg, 17.6%).

C-32: ¹H NMR (400 MHz, CDCl₃) δ_{H} 5.26 (d, J=5.6 Hz, 1H), 3.64-3.60 (m, 1H), 2.55-2.34 (m, 3H), 2.25-2.11 (m, 3H), 2.02-1.56 (m, 11H), 1.50-1.27 (m, 11H), 1.24 (s, 3H), 1.22-1.09 (m, 3H), 0.96-0.92 (m, 6H), 0.60 (s, 3H). LC-ELSD/MS 30-90AB_2 min_E, purity>99%, MS ESI calcd. for C₂₉H₄₄NO [M−H₂O+H]⁺ 422.4, found 422.4.

C-33: ¹H NMR (400 MHz, CDCl₃) δ_{H} 5.26 (d, J=6.0 Hz, 1H), 3.02-2.98 (m, 1H), 2.22-2.11 (m, 1H), 2.03-1.84 (m, 5H), 1.79-1.56 (m, 8H), 1.53-1.27 (m, 11H), 1.24 (s, 3H), 1.23-0.99 (m, 6H), 0.98-0.94 (m, 6H), 0.60 (s, 3H). LC-ELSD/MS 30-90AB 2 min_E, purity>99%, MS ESI calcd. for C₂₉H₄₄NO [M−H₂O+H]⁺ 422.4, found 422.4.

### Example 48: Synthesis of (3R,6R)-3-hydroxy-6-((3S,5S,8S,10S,13R,14S,17R)-3-hydroxy-3,10,13-trimethyl-2,3,4,5,6,7,8,10,12,13,14,15,16,17-tetradecahydro-1H-cyclopenta[a]phenanthren-17-yl)-2,2-dimethylheptanenitrile (C-34) and (3S,6R)-3-hydroxy-6-((3S,5S,8S,10S,13R,14S,17R)-3-hydroxy-3,10,13-trimethyl-2,3,4,5,6,7,8,10,12,13,14,15,16,17-tetradecahydro-1H-cyclopenta[a]phenanthren-17-yl)-2,2-dimethylheptanenitrile (C-35)

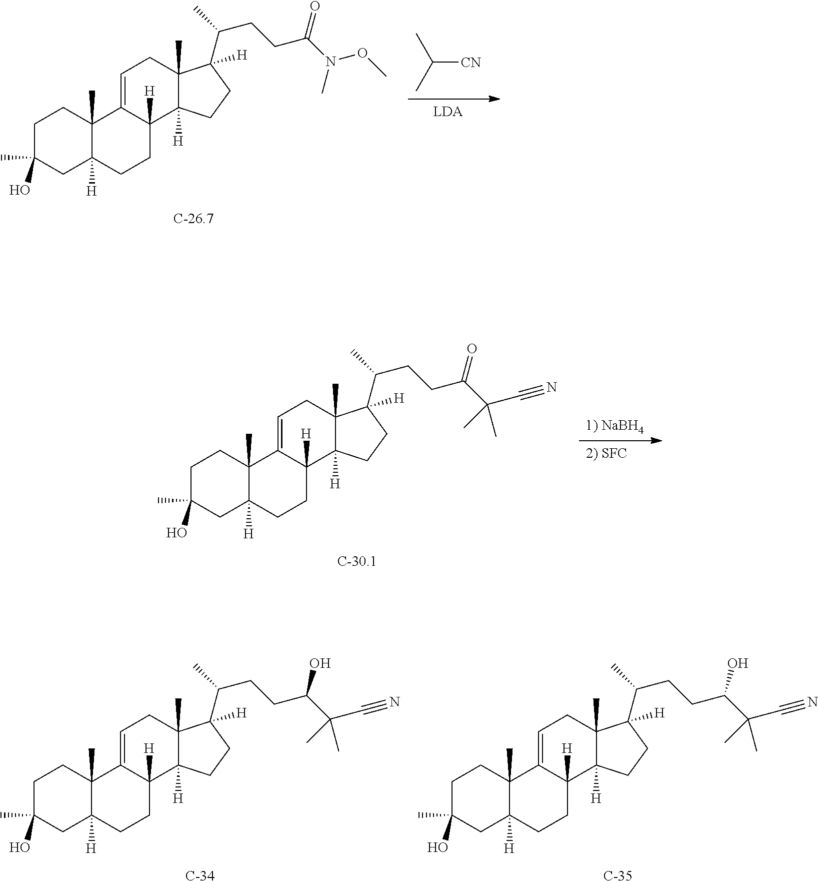

### Synthesis of C-30.1

2-methylpropanenitrile (397 mg, 5.75 mmol) was added to freshly prepared LDA (5.75 mmol, in 5 ml THF) at -60° C. under N₂ and the resulting mixture was stirred under N₂ for 1 h. A solution of C-26.7 (500 mg, 1.15 mmol) in THF (5 mL) was added to the mixture at −60° C. under N₂ and the mixture was warmed to room temperature. NH₄Cl (sat. aq., 50 mL) was added and the aqueous phase was extracted with DCM (2×50 mL). The combined organic layers were washed with brine (2×100 mL), dried over anhydrous Na₂SO₄, filtered, and concentrated in vacuo. The residue was purified by silica gel chromatography (0-40% EtOAc in PE) to give C-30.1 (510 mg, 100%).

### Synthesis of C-34 and C-35

To a solution of C-30.1 (612 mg, 1.40 mmol) in MeOH (6 mL) was added NaBH₄ (79.0 mg, 2.09 mmol) at 20° C. and the resulting mixture was stirred for 2 h. The residue was poured into NH₄Cl (30 mL, sat. aq.) and the aqueous phase was extracted with EtOAc (3×50 mL). The combined organic layers were washed with brine (2×50 mL), dried over anhydrous Na₂SO₄, filtered, and concentrated. The residue was purified by silica gel chromatography (0˜40% of EtOAc in PE) to give C-34 and C-35 (480 mg, 78.0%). C-34 and C-35 (320 mg, 0.7517 mmol) were further purified by SFC (DAICEL CHIRALPAK AD (250 mm*30 mm, 10 um)); Condition: 0.1% NH₃H₂O ETOH; Begin B: 30; End B: 30; FlowRate (ml/min): 70; Injections: 60) to give C-34 (153 mg, 46.3%) and C-35 (165 mg, 50.0%). C-34 (153 mg) and C-35 (165 mg) were independently lyophilized to give C-34 (77.0 mg, 50.6%) and C-35 (132.4 mg, 80.4%), respectively.

C-34: ¹H NMR (400 MHz, CDCl₃) δ_{H} 5.26 (d, J=6.0 Hz, 1H), 3.36 (d, J=8.3 Hz, 1H), 2.20-2.11 (m, 1H), 2.03-1.59 (m, 12H), 1.52-1.39 (m, 6H), 1.38 (s, 3H), 1.34 (s, 3H), 1.32-1.27 (m, 3H), 1.24 (s, 3H), 1.24-1.06 (m, 5H), 0.96-0.93 (m, 6H), 0.60 (s, 3H). LC-ELSD/MS 30-90AB_2 min_E, purity>99%, MS ESI calcd. for C₂₉H₄₆NO [M−H₂O+H]⁺ 424.4, found 424.4.

C-35: ¹H NMR (400 MHz, CDCl₃) δ_{H} 5.26 (d, J=6.0 Hz, 1H), 3.44-3.38 (m, 1H), 2.21-2.11 (m, 1H), 2.03-1.56 (m, 13H), 1.38 (s, 6H), 1.34 (s, 3H), 1.32-1.27 (m, 3H), 1.24 (s, 6H), 1.21-1.09 (m, 3H), 0.97-0.95 (m, 4H), 0.94-0.87 (m, 3H), 0.60 (s, 3H). LC-ELSD/MS 30-90AB_2 min_E, purity>99%, MS ESI calcd. for C₂₉H₄₆NO [M−H₂O+H]⁺ 424.4, found 424.4.

### Example 50: (1R,3aS,3bR,5aS,8R,10aS,10bS,12aR)-8-ethyl-10a,12a-dimethyl-1-((2R,5R)-6,6,6-trifluoro-5-hydroxy-5-methylhexan-2-yl)octadecahydrocyclohepta[a]cyclopenta[f]naphthalen-8-ol (D-1); (1R,3aS,3bR,5aS,8S,10aS,10bS,12aR)-8-ethyl-10a,12a-dimethyl-1-((2R,5R)-6,6,6-trifluoro-5-hydroxy-5-methylhexan-2-yl)octadecahydrocyclohepta[a]cyclopenta[f]naphthalen-8-ol (D-2); (1R,3aS,3bR,5aS,8R,10aS,10bS,12aR)-8-ethyl-10a,12a-dimethyl-1-((2R,5S)-6,6,6-trifluoro-5-hydroxy-5-methylhexan-2-yl)octadecahydrocyclohepta[a]cyclopenta[f]naphthalen-8-ol (D-3); and (1R,3aS,3bR,5aS,8S,10aS,10bS,12aR)-8-ethyl-10a,12a-dimethyl-1-((2R,5S)-6,6,6-trifluoro-5-hydroxy-5-methylhexan-2-yl)octadecahydrocyclohepta[a]cyclopenta[f]naphthalen-8-ol (D-4)

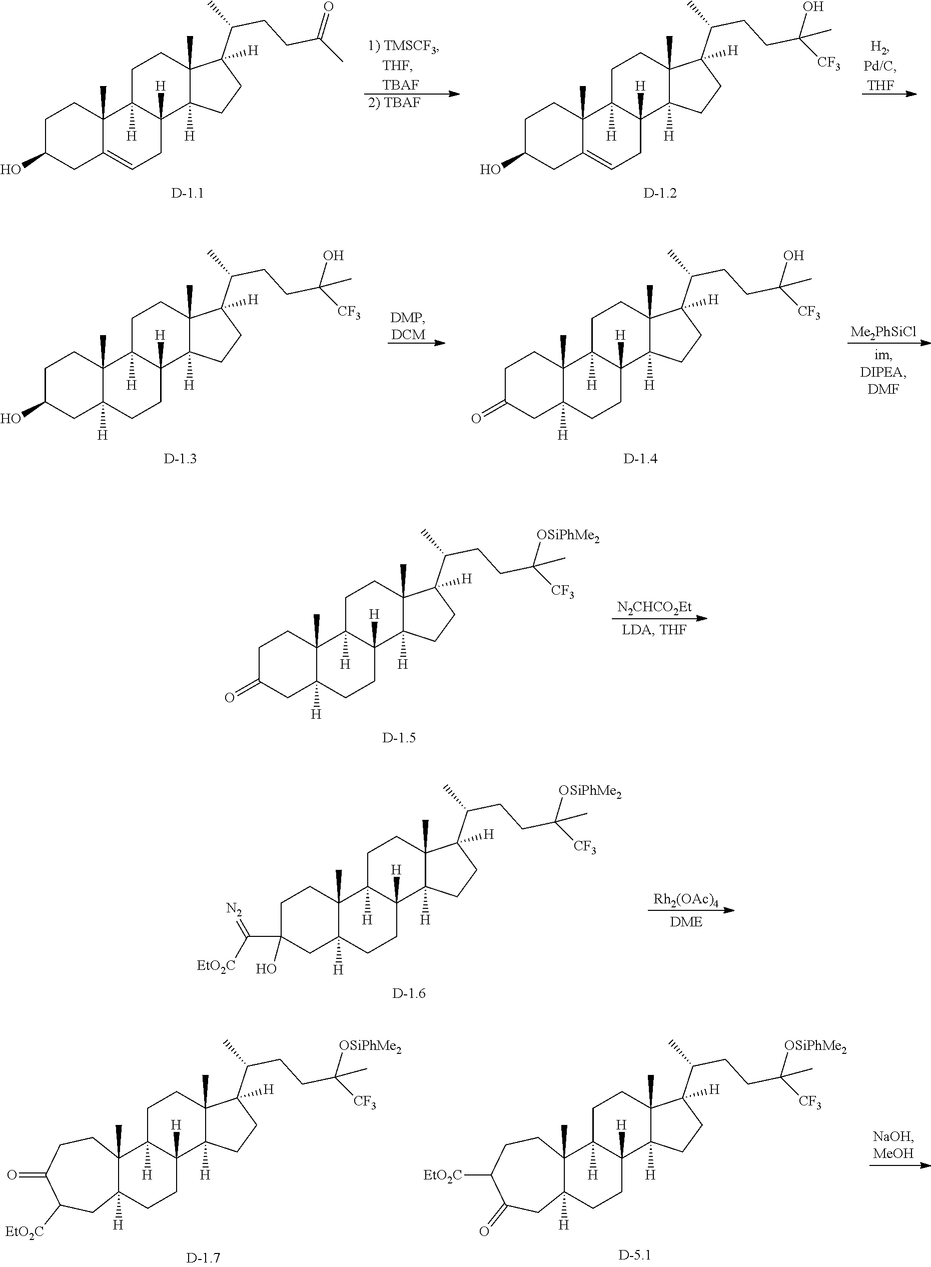

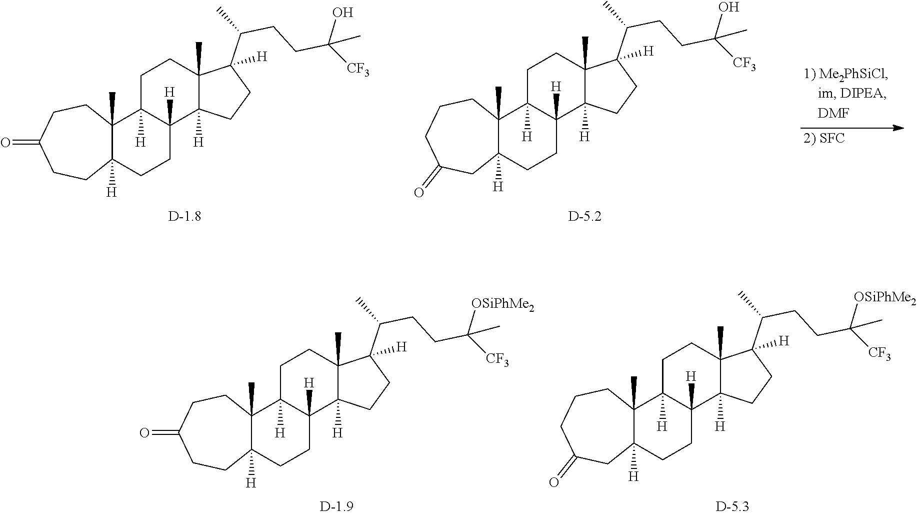

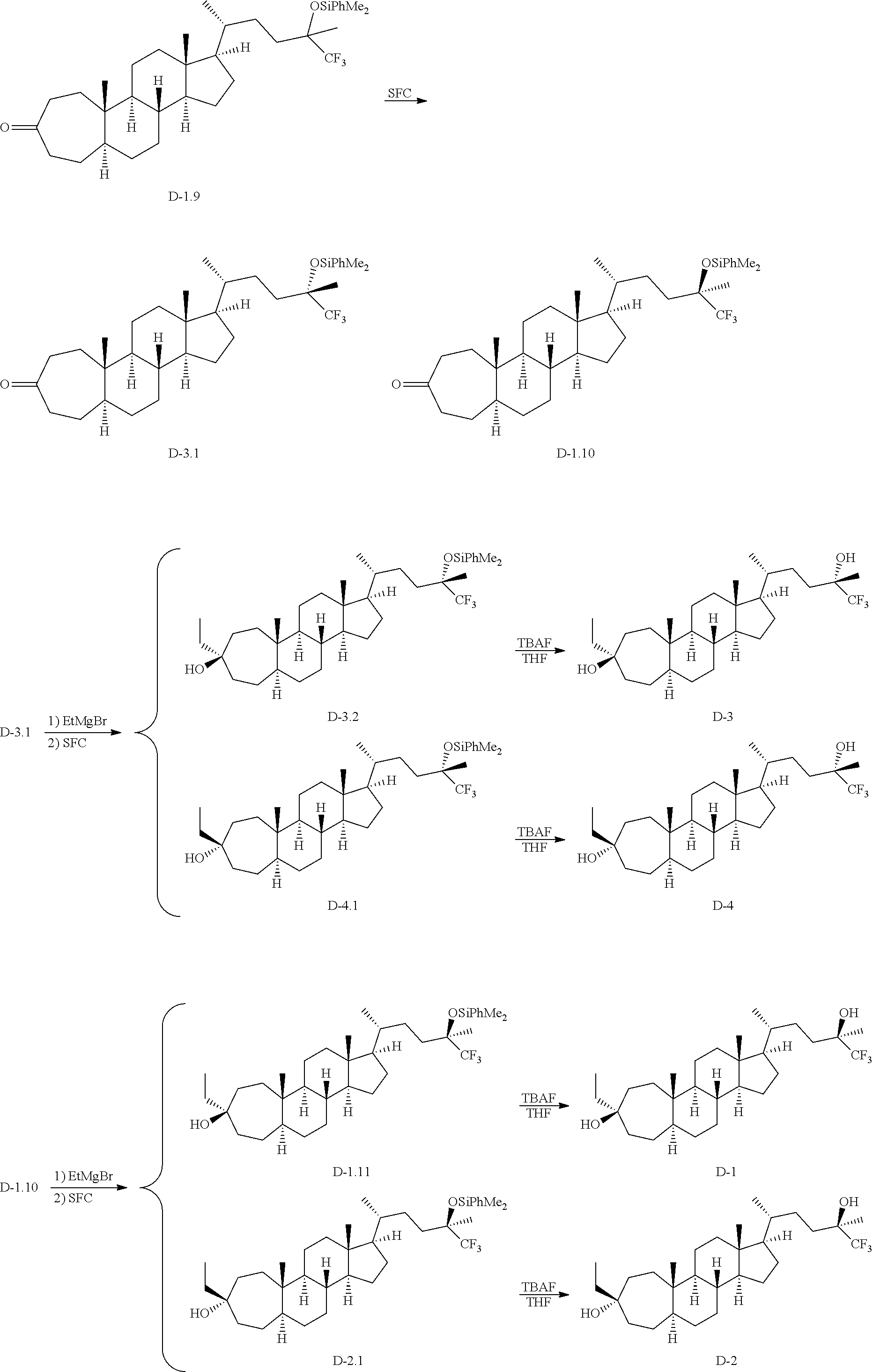

### Synthesis of D-1.2

To a solution of D-1.1 (41.0 g, 110 mmol) in THF (500 mL) was added TBAF (11 mL, 1M, 11 mmol) at 20° C. under N₂. TMSCF₃ (62.5 g, 440 mmol) was added dropwise and the mixture was stirred for 2 hours followed by addition of TBAF (550 mL, 1 M, 550 mmol). The mixture was stirred for another 2 hours and then NH₄Cl (800 mL) was added. The mixture was extracted with EtOAc (2×300 mL), washed with water (100 mL), brine (500 mL), dried over Na₂SO₄, filtered, and concentrated. The product was combined with the product of another batch of D-1.2 and purified by silica gel chromatography (0-30% of EtOAc in PE) to give D-1.2 (45.0 g, 92.5%).

### Synthesis of D-1.3

To a solution of D-1.2 (16.0 g, 36.1 mmol) in THF (200 mL) was added Pd/C (dry, 3.00 g) under N₂. The suspension was degassed under vacuum, purged with H₂ three times, and the mixture was stirred under H₂ (15 psi) at 25° C. for 24 hours. The reaction mixture was filtered through a pad of Celite, washed with THF (2×200 mL), and concentrated to give D-1.3 (15.5 g, crude). ¹H NMR (400 MHz, CDCl₃) δ_{H} 3.67-3.53 (m, 1H), 1.99-1.91 (m, 1H), 1.83-1.77 (m, 2H), 1.74-1.62 (m, 4H), 1.57-1.52 (m, 5H), 1.51-1.46 (m, 2H), 1.42-1.37 (m, 2H), 1.35-1.30 (m, 3H), 1.28-1.19 (m, 5H), 1.16-1.04 (m, 4H), 1.04-0.94 (m, 3H), 0.94-0.89 (m, 3H), 0.89-0.82 (m, 1H), 0.80 (s, 3H), 0.65 (s, 3H), 0.63-0.58 (m, 1H).

### Synthesis of D-1.4

To a solution of D-1.3 (12.5 g, 28.1 mmol) in DCM (120 mL) was added DMP (35.7 g, 84.3 mmol) and the mixture was stirred at 25° C. for 50 minutes. The mixture was quenched with saturated NaHCO₃/Na₂S₂O₃ (1:1, 2×300 mL). The DCM phase was separated and washed with brine (50 mL), dried over Na₂SO₄, filtered, and concentrated to give D-1.4 (13.3 g). ¹H NMR (400 MHz, CDCl₃) δ_{H} 2.48-2.29 (m, 2H), 2.12-1.97 (m, 3H), 1.85-1.78 (m, 2H), 1.77-1.66 (m, 2H), 1.55-1.47 (m, 4H), 1.46-1.34 (m, 5H), 1.34-1.30 (m, 3H), 1.30-1.20 (m, 3H), 1.20-1.08 (m, 4H), 1.08-1.02 (m, 2H), 1.01 (s, 3H), 0.95-0.90 (m, 3H), 0.90-0.81 (m, 1H), 0.77-0.70 (m, 1H), 0.68 (s, 3H).

### Synthesis of D-1.5

To a solution of D-1.4 (13.3 g, 36.8 mol) in THF/DMF (160 ml, 1:1) was added 1H-imidazole (3.75 g, 55.1 mmol), DIPEA (7.11 g, 55.1 mmol), chlorodimethylphenylsilane (12.5 g, 73.6 mmol), and the mixture was stirred at 25° C. for 16 hours. The mixture was poured into aq. NaHCO₃ (100 mL) and extracted with DCM (3×100 mL). The combined organic phase was washed with saturated brine (100 mL), dried over anhydrous Na₂SO₄, filtered, and concentrated. The residue was purified by flash column chromatography (0˜30% of EtOAc in PE) to give D-1.5 (24.7 g). ¹H NMR (400 MHz, CDCl₃) δ_{H} 7.62-7.54 (m, 2H), 7.50-7.30 (m, 3H), 2.49-2.19 (m, 3H), 2.12-1.90 (m, 3H), 1.84-1.58 (m, 4H), 1.54-1.46 (m, 3H), 1.42-1.30 (m, 7H), 1.30-1.27 (m, 3H), 1.27-1.04 (m, 7H), 1.01 (s, 3H), 0.88 (d, J=6.4 Hz, 3H), 0.77-0.68 (m, 1H), 0.66 (s, 3H), 0.50-0.25 (m, 6H).

### Synthesis of D-1.6

A solution of LDA (56 mL, 2.0 M, 112 mmol) was added to a mixture of D-1.5 (13.0 g, 22.5 mmol) and ethyl diazoacetate (12.7 g, 112 mmol) in THF (650 mL) at −70° C. The mixture was stirred at −70° C. for 2 hours and then acetic acid (6.72 g, 112 mmol) in THF (100 mL) was added. The mixture was warmed to 20° C. and stirred for 16 hours. Water (500 mL) and PE (300 mL) were added, the organic phase was separated, and the aqueous phase was extracted with EtOAc (100 mL). The combined organic layers were washed with brine (100 mL), dried over Na₂SO₄, filtered, and concentrated to give the product D-1.6 (18.8 g, crude). ¹H NMR (400 MHz, CDCl₃) δ_{H} 7.54-7.47 (m, 2H), 7.45-7.30 (m, 3H), 4.09-4.02 (m, 1H), 3.42-3.25 (m, 1H), 1.91-1.77 (m, 2H), 1.73-1.64 (m, 2H), 1.63-1.51 (m, 9H), 1.43-1.24 (m, 11H), 1.08-0.89 (m, 8H), 0.83-0.77 (m, 6H), 0.72 (s, 3H), 0.56 (s, 3H), 0.50-0.25 (m, 6H).

### Synthesis of D-1.7 and D-5.1

To a solution of D-1.6 (18.8 g, 27.2 mmol) in DME (180 mL) was added Rh₂(OAc)₄ (240 mg, 0.544 mmol) and the mixture was stirred at 25° C. for 16 hours. The mixture was concentrated to give the product D-1.7 and D-5.1 (18.0 g). ¹H NMR (400 MHz, MeOD) δ_{H} 7.60-7.45 (m, 2H), 7.44-7.31 (m, 3H), 4.22-4.14 (m, 2H), 2.89-2.35 (m, 2H), 2.19-2.06 (m, 1H), 2.00-1.89 (m, 2H), 1.80-1.69 (m, 3H), 1.68-1.50 (m, 12H), 1.40-1.32 (m, 6H), 1.25 (s, 3H), 1.23-1.16 (m, 2H), 1.16-1.02 (m, 5H), 1.01-0.95 (m, 2H), 0.90-0.79 (m, 2H), 0.80 (s, 1H), 0.65-0.60 (m, 3H), 0.55-0.33 (m, 6H).

### Synthesis of D-1.8 and D-5.2

To a mixture of D-1.7 and D-5.1 (18.0 g, 27.1 mmol) in MeOH (200 mL) was added H₂O (60 mL) and NaOH (10.8 g, 271 mmol). The mixture was stirred at 60° C. for 16 hours and then concentrated. H₂O (200 mL) was added and the mixture was extracted with EtOAc (3×300 mL). The combined organic phase was washed with saturated brine (150 mL), dried over anhydrous Na₂SO₄, filtered, and concentrated. The residue was purified by silica gel chromatography (0-60% of EtOAc in PE) to give the product D-1.8 and D-5.2 (13.0 g, 81.2%).

### Synthesis of D-1.9 & D-5.3

To a solution of D-1.8 and D-5.2 (13.0 g, 28.4 mol) in THF/DMF (130 ml, 1:1) was added 1H-imidazole (3.86 g, 56.8 mmol), DIPEA (7.33 g, 56.8 mmol) and chlorodimethylphenylsilane (14.5 g, 85.2 mmol) and the mixture was stirred at 25° C. for 16 hours. The mixture was poured into aq. NaHCO₃ (100 mL), then extracted with DCM (3×200 mL). The combined organic phase was washed with saturated brine (200 mL), dried over anhydrous Na₂SO₄, filtered, and concentrated. The residue was purified by flash column chromatography (0˜30% of EtOAc in PE) to give D-1.9 & D-5.3 (6.60 g, 39.5%). Further purification by SFC (Column: DAICEL CHIRALPAK AS (250 mm*50 mm, 10 um); Condition: 0.1% NH₃H₂O ETOH; Begin B: 35; End B: 35; Gradient Time (min): N/A; 100% B Hold Time (min): N/A) provided D-1.9 (2.00 g, 25%) and D-5.3 (3.00 g, 37.6%).

D-1.9: ¹H NMR (400 MHz, CDCl₃) δ_{H} 7.66-7.50 (m, 2H), 7.42-7.30 (m, 3H), 2.61-2.39 (m, 3H), 2.36-2.25 (m, 1H), 2.03-1.91 (m, 2H), 1.82-1.60 (m, 5H), 1.54-1.45 (m, 2H), 1.43-1.30 (m, 7H), 1.30-1.24 (m, 3H), 1.24-1.10 (m, 4H), 1.10-0.89 (m, 5H), 0.87 (d, J=6.8 Hz, 3H), 0.80 (s, 3H), 0.76-0.68 (m, 1H), 0.64 (s, 3H), 0.50-0.35 (m, 6H).

D-5.3: ¹H NMR (400 MHz, CDCl₃) δ_{H} 7.63-7.51 (m, 2H), 7.44-7.30 (m, 3H), 2.80 (dd, J=11.2, 15.2 Hz, 1H), 2.50-2.27 (m, 2H), 1.99-1.89 (m, 2H), 1.83-1.60 (m, 6H), 1.55-1.46 (m, 3H), 1.44-1.31 (m, 4H), 1.31-1.27 (m, 3H), 1.27-1.13 (m, 4H), 1.13-1.02 (m, 4H), 1.02-0.89 (m, 3H), 0.88-0.81 (m, 6H), 0.78-0.69 (m, 1H), 0.63 (s, 3H), 0.55-0.33 (m, 6H).

### Separation of D-1.10 & D-3.1

D-1.9 was separated by SFC (Column: Phenomenex-Cellulose-2 (250 mm*30 mm, 10 um); Condition: 0.1% NH₃H₂O MEOH; Begin B: 25; End B: 25; FlowRate (mL/min): 60; Injections: 1300) to afford D-1.10 (1.10 g, 55%) and D-3.1 (0.90 g, 45%).

D-1.10: ¹H NMR (400 MHz, CDCl₃) δ_{H} 7.63-7.54 (m, 2H), 7.43-7.31 (m, 3H), 2.60-2.39 (m, 3H), 2.35-2.25 (m, 1H), 2.03-1.91 (m, 2H), 1.81-1.67 (m, 3H), 1.50-1.15 (m, 17H), 1.13-0.91 (m, 5H), 0.91-0.82 (m, 4H), 0.80 (s, 3H), 0.76-0.68 (m, 1H), 0.64 (s, 3H), 0.42 (s, 3H), 0.41 (s, 3H). ¹⁹F NMR (376 MHz, CDCl₃) δ_{F} −81.33 (s)

D-3.1: ¹H NMR (400 MHz, CDCl₃) δ_{H} 7.63-7.53 (m, 2H), 7.42-7.30 (m, 3H), 2.60-2.38 (m, 3H), 2.34-2.25 (m, 1H), 2.03-1.90 (m, 2H), 1.81-1.62 (m, 4H), 1.54-1.18 (m, 15H), 1.16-0.94 (m, 6H), 0.91-0.84 (m, 4H), 0.80 (s, 3H), 0.76-0.68 (m, 1H), 0.64 (s, 3H), 0.42 (s, 3H), 0.41 (s, 3H). ¹⁹F NMR (376 MHz, CDCl₃) δ_{F} −81.70 (s)

### Synthesis of D-3.2 & D-4.1

A solution of EtMgBr (2.25 mL, 3 M, 6.76 mmol) in THF (6 mL) was reacted with D-3.1 (200 mg, 0.34 mmol) in THF (2 mL) at 20° C. under N₂ and the mixture was stirred for 3 hours. The mixture was quenched with saturated NH₄Cl (10 mL) and extracted with EtOAc (2×10 mL). The combined organic phase was washed with water (3×10 mL), dried over Na₂SO₄, filtered, and concentrated. Separation by SFC (Column: DAICEL CHIRALCEL OD-H (250 mm*30 mm, 5 um); Condition: 0.1% NH₃H₂O ETOH; Begin B: 25; End B: 25; FlowRate (ml/min): 60; Injections: 80) afforded D-3.2 (50.0 mg, 25%) and D-4.1 (90.0 mg, 45%).

D-3.2: ¹H NMR (400 MHz, CDCl₃) δ_{H} 7.63-7.51 (m, 2H), 7.47-7.30 (m, 3H), 1.99-1.88 (m, 1H), 1.79-1.59 (m, 8H), 1.51-1.11 (m, 17H), 1.10-0.94 (m, 8H), 0.93-0.85 (m, 7H), 0.79 (s, 3H), 0.73-0.65 (m, 1H), 0.63 (s, 3H), 0.42 (s, 3H), 0.41 (s, 3H). ¹⁹F NMR (376 MHz, CDCl₃) δ_{F} −81.71 (s)

D-4.1: ¹H NMR (400 MHz, CDCl₃) δ_{H} 7.64-7.53 (m, 2H), 7.42-7.30 (m, 3H), 2.60-2.39 (m, 1H), 2.34-2.23 (m, 1H), 2.04-1.86 (m, 2H), 1.79-1.59 (m, 6H), 1.52-1.18 (m, 16H), 1.17-0.97 (m, 8H), 0.94-0.82 (m, 8H), 0.74 (s, 3H), 0.65-0.60 (m, 3H), 0.42 (s, 3H), 0.41 (s, 3H). ¹⁹F NMR (376 MHz, CDCl₃) δ_{F} −81.72 (s)

### Synthesis of D-3

To a suspension of D-3.2 (50.0 mg, 0.08 mmol) in THF (1 mL) was added TBAF (0.16 mL, 0.16 mmol) and the mixture was stirred at 20° C. for 16 hours. The mixture was quenched with saturated NH₄Cl (10 mL) and extracted with EtOAc (2×10 mL). The combined organic phase was washed with water (3×10 mL), dried over Na₂SO₄, filtered, and concentrated. Purification by flash column chromatography (0˜20% of EtOAc in PE) provided D-3 (33.5 mg, 86%). ¹H NMR (400 MHz, CDCl₃) δ_{H} 1.98-1.90 (m, 1H), 1.88-1.56 (m, 11H), 1.53-1.16 (m, 15H), 1.15-0.99 (m, 8H), 0.94-0.83 (m, 7H), 0.80 (s, 3H), 0.74-0.63 (m, 4H). ¹⁹F NMR (376 MHz, CDCl₃) δ_{F} −83.13 (s). LC-ELSD/MS 30-90AB_2 min_E, purity >99%, MS ESI calcd. for C₂₉H₄₉F₃O₂ [M−H₂O+H]⁺ 469.4, found 469.4.

### Synthesis of D-4

To a suspension of D-4.1 (60.0 mg, 0.10 mmol) in THF (1 mL) was added TBAF (0.19 mL, 0.19 mmol) and the mixture was stirred at 20° C. for 16 hours. The mixture was quenched with NH₄Cl (10 mL) and extracted with EtOAc (2×10 mL). The combined organic phase was washed with water (3×10 mL), dried over Na₂SO₄, filtered, and concentrated. Purification by flash column chromatography (0˜25% of EtOAc in PE) provided D-4 (34.5 mg, 73%). ¹H NMR (400 MHz, CDCl₃) δ_{H} 1.98-1.56 (m, 11H), 1.51-1.23 (m, 15H), 1.21-1.00 (m, 9H), 0.94-0.84 (m, 7H), 0.76-0.69 (m, 4H), 0.65 (s, 3H). ¹⁹F NMR (376 MHz, CDCl₃) δ_{F} −83.15 (s). LC-ELSD/MS 30-90AB_2 min_E, purity >99%, MS ESI calcd. for C₂₉H₄₉F₃O₂ [M−H₂O+H]⁺ 469.4, found 469.4.

### Synthesis of D-1.11 & D-2.1

A solution of EtMgBr (2.25 mL, 3 M, 6.76 mmol) in THF was reacted with D-1.10 (200 mg, 0.34 mmol) at 20° C. under N₂ and the mixture was stirred for 4 hours. The mixture was quenched with NH₄Cl (10 mL) and extracted with EtOAc (2×10 mL). The combined organic phase was washed with water (3×10 mL), dried over Na₂SO₄, filtered, and concentrated. Purification by SFC (Column: DAICEL CHIRALPAK AD (250 mm*30 mm, 10 um); Condition: 0.1% NH₃H₂O ETOH; Begin B: 20; End B: 20; FlowRate (mL/min): 60; Injections: 60) provided D-2.1 (80.0 mg, 40%) and D-1.11 (50.0 mg, 25%).

D-1.11: ¹H NMR (400 MHz, CDCl₃) δ_{H} 7.62-7.50 (m, 2H), 7.43-7.30 (m, 3H), 1.97-1.85 (m, 2H), 1.82-1.69 (m, 2H), 1.67-1.61 (m, 2H), 1.55-1.20 (m, 18H), 1.20-0.96 (m, 10H), 0.93-0.84 (m, 7H), 0.77-0.68 (m, 4H), 0.63 (s, 3H), 0.42 (s, 3H), 0.41 (s, 3H). ¹⁹F NMR (376 MHz, CDCl₃) δ_{F} −81.34 (s)

D-2.1: ¹H NMR (400 MHz, CDCl₃) δ_{H} 7.61-7.53 (m, 2H), 7.42-7.31 (m, 3H), 1.96-1.88 (m, 1H), 1.81-1.60 (m, 7H), 1.53-1.15 (m, 17H), 1.15-0.95 (m, 9H), 0.93-0.84 (m, 7H), 0.79 (s, 3H), 0.74-0.66 (m, 1H), 0.63 (s, 3H), 0.42 (s, 3H), 0.41 (s, 3H). ¹⁹F NMR (376 MHz, CDCl₃) δ_{F} −81.35 (s)

### Synthesis of D-1

To a suspension of D-1.11 (50.0 mg, 0.08 mmol) in THF (1 mL) was added TBAF (0.16 mL, 0.16 mmol) and the mixture was stirred at 20° C. for 16 hours. The mixture was quenched with NH₄Cl (10 mL) and extracted with EtOAc (2×10 mL). The combined organic phase was washed with water (3×10 mL), dried over Na₂SO₄, filtered, and concentrated. Purification by flash column chromatography (0˜15% of EtOAc in PE) provided D-1 (29.4 mg, 59%). ¹H NMR (400 MHz, CDCl₃) δ_{H} 1.97-1.90 (m, 1H), 1.86-1.60 (m, 8H), 1.53-1.28 (m, 13H), 1.28-0.98 (m, 13H), 0.94-0.84 (m, 7H), 0.80 (s, 3H), 0.74-0.63 (m, 4H). ¹⁹F NMR (376 MHz, CDCl₃) δ_{F} −82.83 (s). LC-ELSD/MS 30-90AB_2 min_E, purity 99%, MS ESI calcd. for C₂₉H₄₉F₃O₂ [M−H₂O+H]⁺ 469.4, found 469.4.

### Synthesis of D-2

To a suspension of D-2.1 (80.0 mg, 0.13 mmol) in THF (1 mL) was added TBAF (0.26 mL, 0.26 mmol) and the mixture was stirred at 20° C. for 16 hours. The mixture was quenched with saturated NH₄Cl (10 mL) and extracted with EtOAc (2×10 mL). The combined organic phase was washed with water (3×10 mL), dried over Na₂SO₄, filtered, and concentrated. Purification by flash column chromatography (0˜15% of EtOAc in PE) provided D-2 (45.6 mg, 57%). ¹H NMR (400 MHz, CDCl₃) δ_{H} 1.97-1.60 (m, 9H), 1.53-1.36 (m, 8H), 1.35-1.30 (m, 4H), 1.29-0.99 (m, 14H), 0.94-0.86 (m, 7H), 0.76-0.69 (m, 4H), 0.65 (s, 3H). ¹⁹F NMR (376 MHz, CDCl₃) δ_{F} −82.82 (s). LC-ELSD/MS 30-90AB_2 min_E, purity >99%, MS ESI calcd. for C₂₉H₄₉F₃O₂ [M−H₂O+H]⁺ 469.4, found 469.4.

### Example 51: (1R,3aS,3bR,5aS,7S,10aS,10bS,12aR)-7-ethyl-10a,12a-dimethyl-1-((2R,5R)-6,6,6-trifluoro-5-hydroxy-5-methylhexan-2-yl)octadecahydrocyclohepta[a]cyclopenta[f]naphthalen-7-ol (D-5); (1R,3aS,3bR,5aS,7R,10aS,10bS,12aR)-7-ethyl-10a,12a-dimethyl-1-((2R,5R)-6,6,6-trifluoro-5-hydroxy-5-methylhexan-2-yl)octadecahydrocyclohepta[a]cyclopenta[f]naphthalen-7-ol (D-6); (1R,3aS,3bR,5aS,7S,10aS,10bS,12aR)-7-ethyl-10a,12a-dimethyl-1-((2R,5S)-6,6,6-trifluoro-5-hydroxy-5-methylhexan-2-yl)octadecahydrocyclohepta[a]cyclopenta[f]naphthalen-7-ol (D-7); (1R,3aS,3bR,5aS,7S,10aS,10bS,12aR)-7-ethyl-10a,12a-dimethyl-1-((2R,5S)-6,6,6-trifluoro-5-hydroxy-5-methylhexan-2-yl)octadecahydrocyclohepta[a]cyclopenta[f]naphthalen-7-ol (D-8)

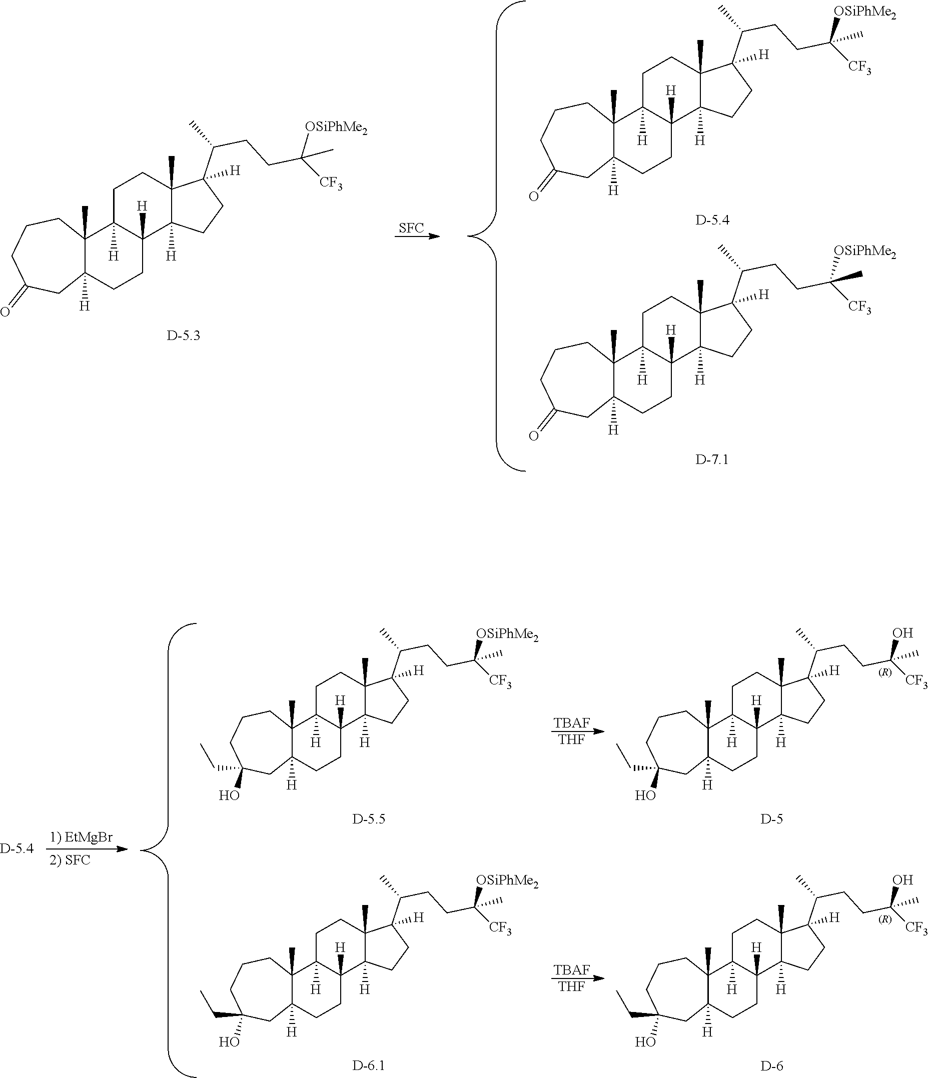

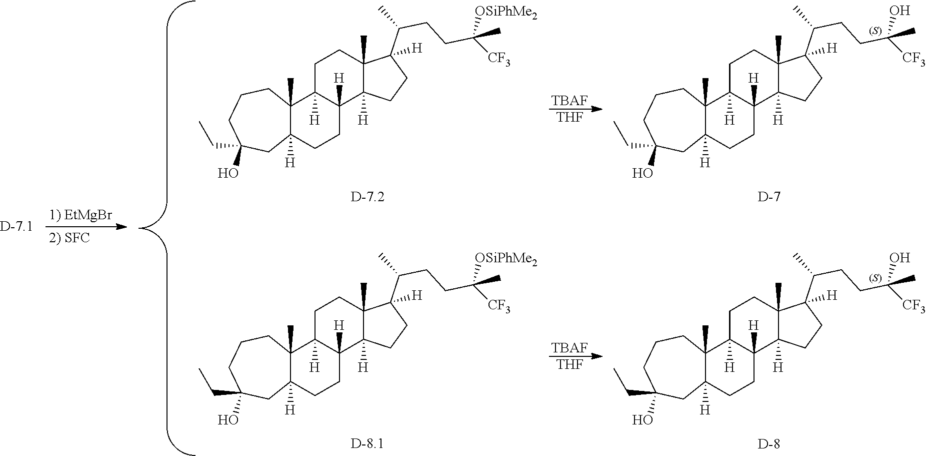

### Separation of D-5.4 and D-7.1

Ketone D-5.3 (3.00 g, 4.5 mmol) was further purified by SFC (Column: Phenomenex Lux Cellulose-4 250*30 mm*5 um; Conditions: 0.1% NH₃H₂O MEOH; Begin B: 15; End B: 15; Gradient Time (min): N/A; 100% B Hold Time (min): N/A) to give D-5.4 (750 mg, 35.8%) and D-7.1 (500 mg).

D-5.4: ¹H NMR (400 MHz, CDCl₃) δ_{H} 7.65-7.50 (m, 2H), 7.42-7.31 (m, 3H), 2.80 (dd, J=11.2, 15.2 Hz, 1H), 2.50-2.30 (m, 2H), 1.99-1.88 (m, 2H), 1.82-1.59 (m, 6H), 1.56-1.34 (m, 7H), 1.29 (s, 3H), 1.28-0.91 (m, 11H), 0.90-0.81 (m, 6H), 0.78-0.69 (m, 1H), 0.64 (s, 3H), 0.54-0.30 (m, 6H). ¹⁹F NMR (376 MHz, CDCl₃) δ_{F} −81.35 (s).

D-7.1: ¹H NMR (400 MHz, CDCl₃) δ_{H} 7.65-7.50 (m, 2H), 7.41-7.31 (m, 3H), 2.80 (dd, J=11.2, 15.2 Hz, 1H), 2.50-2.29 (m, 2H), 2.04-1.91 (m, 3H), 1.81-1.59 (m, 6H), 1.55-1.32 (m, 7H), 1.28 (s, 3H), 1.24-0.89 (m, 9H), 0.88-0.80 (m, 7H), 0.78-0.68 (m, 1H), 0.63 (s, 3H), 0.50-0.33 (m, 6H). ¹⁹F NMR (376 MHz, CDCl₃) δ_{F} −81.72 (s).

### Synthesis of D-5.5 & D-6.1

To a solution of D-5.4 (200 mg, 0.3384 mmol) in THF (5 mL) was added ethyl magnesium bromide (1.12 mL, 3.38 mmol, 3M) and the mixture was stirred at 25° C. for 16 hours. The mixture was poured into saturated NH₄Cl (20 mL), stirred for 10 minutes, and extracted with EtOAc (2×30 mL). The combined organic phase was washed with saturated brine, filtered, and concentrated to give D-5.5 & D-6.1 (200 mg). Purification by SFC (Column: DAICEL CHIRALPAK AD (250 mm*30 mm, 10 um)); Condition: 0.1% NH₃H₂O ETOH; Begin B: 15; End B: 15; Gradient Time (min): n/a; 100% B Hold Time (min): n/a) provided D-5.5 (60.0 mg, 30.1%) and D-6.1 (110 mg, 55.2%).

D-5.5: ¹H NMR (400 MHz, CDCl₃) δ_{H} 7.65-7.52 (m, 2H), 7.44-7.29 (m, 3H), 1.96-1.33 (m, 24H), 1.29 (s, 3H), 1.21-0.93 (m, 11H), 0.90 (t, J=7.6 Hz, 3H), 0.86 (d, J=6.4 Hz, 3H), 0.66-0.62 (m, 1H), 0.62 (s, 3H), 0.53-0.35 (m, 6H). ¹⁹F NMR (376 MHz, CDCl₃) &F-81.35 (s)

D-6.1: ¹H NMR (400 MHz, CDCl₃) δ_{H} 7.65-7.53 (m, 2H), 7.42-7.32 (m, 3H), 1.96-1.89 (m, 1H), 1.81-1.32 (m, 19H), 1.29 (s, 3H), 1.28-0.96 (m, 12H), 0.91-0.84 (m, 6H), 0.75 (s, 3H), 0.74-0.68 (m, 1H), 0.62 (s, 3H), 0.53-0.35 (m, 6H). ¹⁹F NMR (376 MHz, CDCl₃) δ_{F} −81.35 (s).

### Synthesis of D-5

To a suspension of D-5.5 (75.0 mg, 0.1207 mmol) in THF (2 mL) was added TBAF (1.2 mL, 1.2 mmol) and the mixture was stirred at 25° C. for 16 hours. The mixture was quenched with saturated NH₄Cl (10 mL) and extracted with EtOAc (2×20 mL). The combined organic phase was washed with water (2×20 mL), dried over Na₂SO₄, filtered, and concentrated. Purification by flash column chromatography (0˜30% of EtOAc in PE) provided D-5 (33.8 mg, 57.5%). ¹H NMR (400 MHz, CDCl₃) δ_{H} 1.98-1.91 (m, 1H), 1.88-1.59 (m, 9H), 1.53-1.34 (m, 8H), 1.32 (s, 3H), 1.30-0.95 (m, 13H), 0.94-0.83 (m, 8H), 0.81 (s, 3H), 0.64 (s, 3H), 0.63-0.58 (m, 1H). ¹⁹F NMR (376 MHz, CDCl₃) δ_{F} −82.82 (s). LC-ELSD/MS: purity>99%, MS ESI calcd. for C₂₉H₄₉F₃O₂ [M+H−H₂O]⁺ 469.4, found 469.4.

### Synthesis of D-6

To a suspension of D-6.1 (150 mg, 0.2415 mmol) in THF (2 mL) was added TBAF (2.41 mL, 2.41 mmol) and the mixture was stirred at 25° C. for 16 hours. The mixture was quenched with sat. NH₄Cl (10 mL) and extracted with EA (2×20 mL). The combined organic phase was washed with water (2×20 mL), dried over Na₂SO₄, filtered, and concentrated. purification by flash column chromatography (0˜30% of EtOAc in PE) provided D-6 (83.0 mg, 70.9%). ¹H NMR (400 MHz, CDCl₃) δ_{H} 1.97-1.71 (m, 6H), 1.68-1.61 (m, 2H), 1.57-1.34 (m, 10H), 1.32 (s, 3H), 1.31-0.95 (m, 14H), 0.94-0.87 (m, 7H), 0.75 (s, 3H), 0.74-0.69 (m, 1H), 0.64 (s, 3H). ¹⁹F NMR (376 MHz, CDCl₃) δ_{F} −82.83 (s). LC-ELSD/MS: purity >99%, MS ESI calcd. for C₂₉H₄₉F₃O₂ [M+H−H₂O]⁺ 469.4, found 469.3.

### Synthesis of D-7.2 & D-8.1

To a solution of D-7.1 (300 mg, 0.5021 mmol) in THF (5 mL) was added ethyl magnesium bromide (2.03 mL, 6.09 mmol, 3M) and the mixture was stirred at 25° C. for 16 hours. The mixture was poured into saturated NH₄Cl (20 mL), stirred for 10 minutes, and the aqueous phase was extracted with EtOAc (2×30 mL). The organic phase was washed with saturated brine, filtered, and concentrated to give a mixture of D-7.2 & D-8.1 (300 mg). Further purification by SFC (Column: DAICEL CHIRALPAK AD (250 mm*30 mm, 10 um); Condition: 0.1% NH₃H₂O EtOH; Begin B: 15; End B: 15; Gradient Time (min): n/a; 100% B Hold Time (min): n/a) provided D-7.2 (110 mg, 36.7%) and D-8.1 (190 mg, 63.5%).

D-7.2: ¹H NMR (400 MHz, CDCl₃) δ_{H} 7.63-7.50 (m, 2H), 7.42-7.30 (m, 3H), 1.97-1.41 (m, 20H), 1.28 (s, 3H), 1.21-0.94 (m, 10H), 0.93-0.84 (m, 8H), 0.81 (s, 3H), 0.67-0.62 (m, 1H), 0.62-0.57 (m, 3H), 0.55-0.35 (m, 6H). ¹⁹F NMR (376 MHz, CDCl₃) δ_{F} −81.69 (s)

D-8.1: ¹H NMR (400 MHz, CDCl₃) δ_{H} 7.54-7.46 (m, 2H), 7.34-7.22 (m, 3H), 1.89-1.82 (m, 1H), 1.73-1.23 (m, 20H), 1.21 (s, 3H), 1.14-0.88 (m, 9H), 0.87-0.77 (m, 8H), 0.68 (s, 3H), 0.67-0.61 (m, 1H), 0.55 (s, 3H), 0.55-0.30 (m, 6H). ¹⁹F NMR (376 MHz, CDCl₃) δ_{F} −81.71 (s).

### Synthesis of D-7

To a suspension of D-7.2 (110 mg, 0.1771 mmol) in THF (2 mL) was added TBAF (1.77 mL, 1.77 mmol) and the mixture was stirred at 25° C. for 16 hours. The mixture was quenched with saturated NH₄Cl (10 mL) and extracted with EtOAc (2×20 mL). The combined organic phase was washed with water (2×20 mL), dried over Na₂SO₄, filtered, and concentrated. Purification by flash column chromatography (0˜30% of EtOAc in PE) provided D-7 (38.4 mg, 44.5%). ¹H NMR (400 MHz, CDCl₃) δ_{H} 1.92-1.84 (m, 1H), 1.82-1.51 (m, 9H), 1.48-1.26 (m, 8H), 1.24 (s, 3H), 1.23-0.87 (m, 13H), 0.87-0.79 (m, 7H), 0.79-0.75 (m, 1H), 0.74 (s, 3H), 0.57 (s, 3H), 0.56-0.51 (m, 1H). ¹⁹F NMR (376 MHz, CDCl₃) δ_{F} −83.12 (s). LC-ELSD/MS: purity>99%, MS ESI calcd. for C₂₉H₄₉F₃O₂ [M+H−H₂O]⁺ 469.4, found 469.3.

### Synthesis of D-8

To a suspension of D-8.1 (190 mg, 0.3059 mmol) in THF (2 mL) was added TBAF (3.05 mL, 3.05 mmol) and the mixture was stirred at 25° C. for 16 hours. The mixture was quenched with saturated NH₄Cl (10 mL) and extracted with EtOAc (2×20 mL). The combined organic phase was washed with water (2×20 mL), dried over Na₂SO₄, filtered, and concentrated. Purification by flash column chromatography (0˜30% of EtOAc in PE) provided D-8 (97.8 mg, 66%). ¹H NMR (400 MHz, CDCl₃) δ_{H} 1.98-1.91 (m, 1H), 1.88-1.57 (m, 10H), 1.55-1.32 (m, 9H), 1.31 (s, 3H), 1.28-0.97 (m, 11H), 0.95-0.84 (m, 8H), 0.75 (s, 3H), 0.74-0.68 (m, 1H), 0.64 (s, 3H). ¹⁹F NMR (376 MHz, CDCl₃) δ_{F} −83.13 (s). LC-ELSD/MS: purity >99%, MS ESI calcd. for C₂₉H₄₉F₃O₂ [M+H−H₂O]⁺ 469.4, found 469.4.

### Example 52: (1R,3aS,3bS,7S,10aR,10bS,12aR)-7-ethyl-1-((R)-5-hydroxy-5-methylhexan-2-yl)-10a,12a-dimethyl-1,2,3,3a,3b,4,6,7,8,9,10,10a,10b,11,12,12a-hexadecahydrocyclohepta[a]cyclopenta[f]naphthalen-7-ol (D-9)

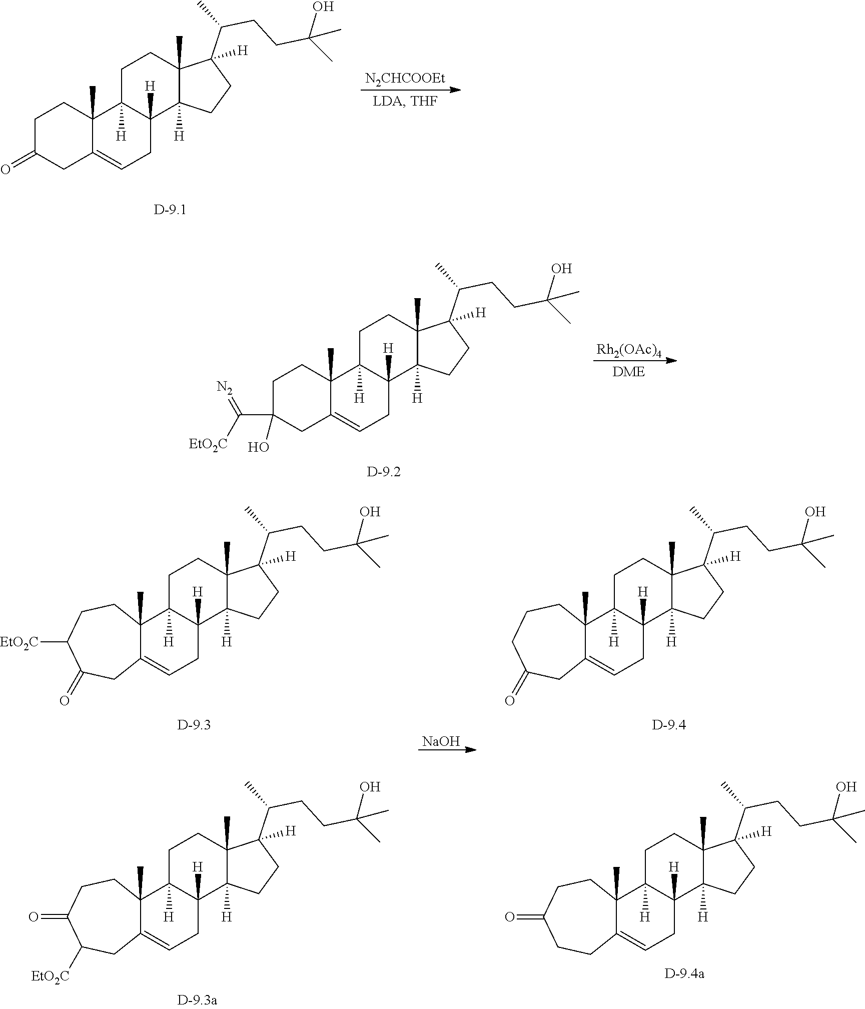

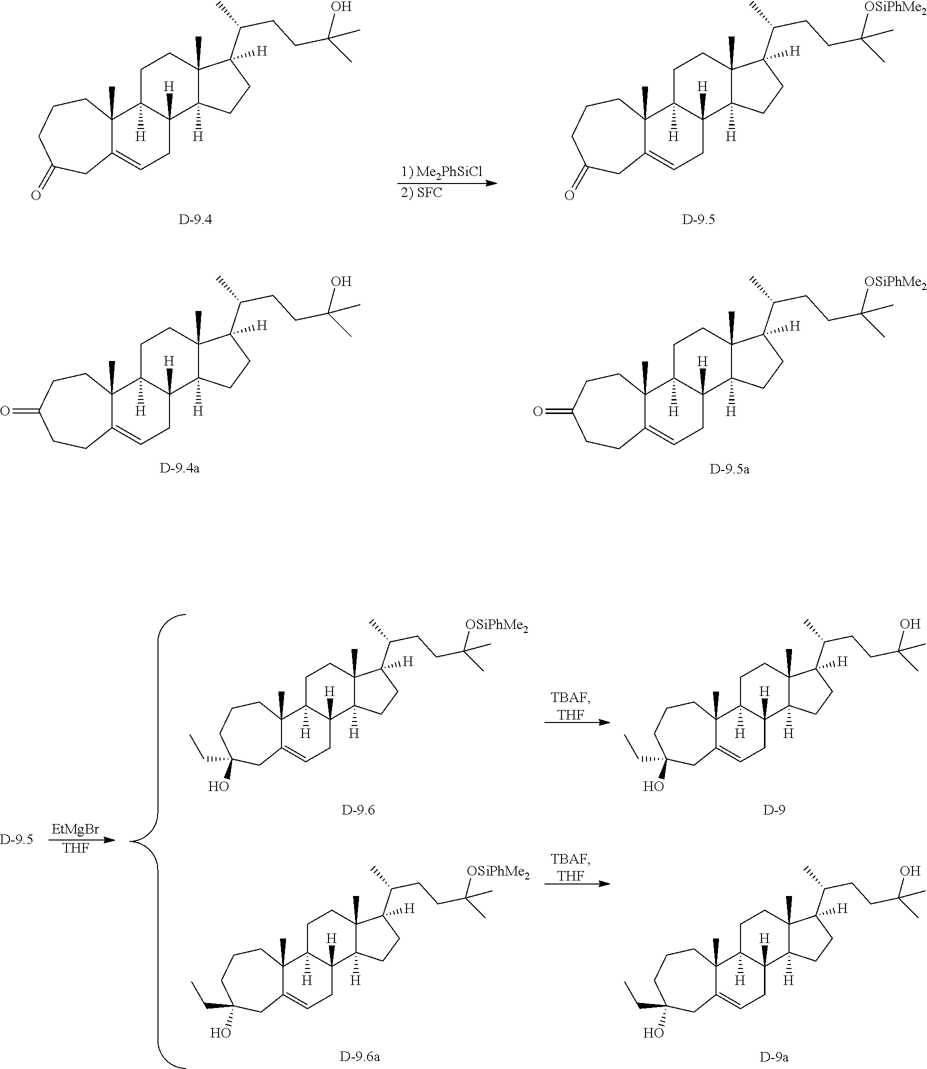

### Synthesis of D-9.2

To a stirred solution of D-9.1 (4.44 g, 11.4 mmol) and ethyl diazoacetate (5.20 g, 45.6 mmol) in THF (250 mL) at -70° C. was added LDA (22.8 mL, 45.6 mmol, 2M) and the mixture was stirred for 2 hours. Acetic acid (2.73 g, 45.6 mmol) in THF (20 mL) was added and the mixture was warmed to 25° C. and stirred for 16 hours. Water (300 mL) and PE (150 mL) were added, the organic phase was separated, and the aqueous phase was extracted with EtOAc (200 mL). The combined organic layers were washed with saturated brine (200 mL), dried over anhydrous Na₂SO₄, filtered, and concentrated to give D-9.2 (8.10 g).

### Synthesis of D-9.3 and D-9.3a

To a solution of D-9.2 (8.10 g, 16.1 mmol) in DME (50 mL) was added Rh₂(OAc)₄ (71.1 mg, 0.161 mmol) and the reaction mixture was stirred at 25° C. for 16 hours. The mixture was concentrated to give a mixture of D-9.3 and D-9.3a (7.00 g, crude) which was used directly in the next step.

### Synthesis of D-9.4 and D-9.4a

To a mixture of D-9.3 and D-9.3a (7.00 g, 14.8 mmol) in MeOH (70 mL) was added H₂O (30 mL) and NaOH (5.91 g, 148 mmol) at 25° C. The reaction mixture was stirred at 60° C. for 16 hours, concentrated, and H₂O (200 mL) was added. The mixture was extracted with EtOAc (3×300 mL) and the combined organic phase was washed with saturated brine (150 mL), dried over anhydrous Na₂SO₄, filtered, and concentrated to give a mixture of D-9.4 and D-9.4a (1.40 g).

### Synthesis of D-9.5 and D-9.5a

To a solution of D-9.4 and D-9.4a (2.93 g, 7.31 mmol) in DCM (5 ml) was added imidazole (993 mg, 14.6 mmol), DIPEA (944 mg, 7.31 mmol), and chlorodimethylphenylsilane (3.73 g, 21.9 mmol), and the reaction mixture was stirred at 25° C. for 16 hours. The mixture was poured into NaHCO₃ (10 mL) and extracted with DCM (3×20 mL). The combined organic phase was washed with saturated brine (30 mL), dried over anhydrous Na₂SO₄, filtered, and concentrated. Purification by SFC (Column: DAICEL CHIRALPAK IC (250 mm*30 mm, 10 um); Condition: 0.1% NH₃H₂O MeOH; Begin B: 35%; End B: 35%; FlowRate (mL/min): 70; Injections: 370) provided D-9.5 (410 mg, 40.1%) and D-9.5a (390 mg, 38.2%).

D-9.5: ¹H NMR (400 MHz, CDCl₃) δ_{H} 7.62-7.57 (m, 2H), 7.38-7.32 (m, 3H), 5.58-5.54 (m, 1H), 3.28-3.22 (m, 1H), 3.19-3.19 (m, 1H), 2.85-2.79 (m, 1H), 2.66-2.56 (m, 1H), 2.24-2.16 (m, 1H), 2.10-1.98 (m, 3H), 1.90-1.80 (m, 2H), 1.65-1.59 (m, 2H), 1.53-1.21 (m, 11H), 1.21-1.15 (m, 6H), 1.13-1.03 (m, 4H), 0.98 (s, 3H), 0.89 (d, J=6.4 Hz, 3H), 0.68 (s, 3H), 0.37 (s, 6H).

D-9.5a: ¹H NMR (400 MHz, CDCl₃) δ_{H} 7.62-7.57 (m, 2H), 7.38-7.31 (m, 3H), 5.57 (m, 1H), 2.61 (m, 1H), 2.49-2.29 (m, 4H), 2.20-2.11 (m, 1H), 2.05-1.95 (m, 2H), 1.88-1.68 (m, 3H), 1.52-1.46 (m, 4H), 1.41-1.23 (m, 5H), 1.22-1.15 (m, 8H), 1.14-1.02 (m, 5H), 1.00 (s, 3H), 0.89 (d, J=6.4 Hz, 3H), 0.68 (s, 3H), 0.36 (s, 6H).

### Synthesis of D-9.6 and D-9.6a

A solution of EtMgBr (1.99 g, 5 mL, 15.0 mmol, 3 M) was reacted with D-9.5 (200 mg, 0.373 mmol) in THF (2.5 mL) at 25° C. under N₂ and the mixture was stirred for 30 minutes. NH₄Cl was added and the mixture was stirred for 5 minutes followed by extraction with EtOAc (3×20 mL). The combined organic phase was washed with saturated brine (2×20 mL), dried over anhydrous Na₂SO₄, filtered, and concentrated. The residue was purified by flash column chromatography (0˜5% of EtOAc in PE) to give D-9.6a (166 mg) and D-9.6 (83.0 mg).

D-9.6a: ¹H NMR (400 MHz, CDCl₃) δ_{H} 7.63-7.56 (m, 2H), 7.35 (m, 3H), 5.53-5.38 (m, 1H), 2.44-2.30 (m, 1H), 2.04-1.82 (m, 5H), 1.52-1.28 (m, 13H), 1.25-1.17 (m, 6H), 1.16-0.87 (m, 20H), 0.69-0.64 (m, 3H), 0.36 (s, 6H).

D-9.6: ¹H NMR (400 MHz, CDCl₃) δ_{H} 7.63-7.56 (m, 2H), 7.35 (m, 3H), 5.44-5.36 (m, 1H), 2.45-2.38 (m, 1H), 2.04-1.74 (m, 5H), 1.65-1.28 (m, 16H), 1.25-1.17 (m, 6H), 1.16-0.94 (m, 8H), 0.93-0.86 (m, 9H), 0.67 (s, 3H), 0.37 (s, 6H).

### Synthesis of D-9

To a suspension of D-9.6 (83.0 mg, 0.146 mmol) in THF (3 mL) was added TBAF (0.292 mL, 0.293 mmol, 1M in THF) and the mixture was stirred at 80° C. for 16 hours. The mixture was cooled, quenched with saturated NH₄Cl (5 mL), and extracted with EtOAc (2×20 mL). The combined organic phase was washed with water (3×30 mL), dried over Na₂SO₄, filtered, and concentrated. Purification by flash column chromatography (0˜30% of EtOAc in PE) followed by lyophilization provided D-9 (9.60 mg, 14.8%). ¹H NMR (400 MHz, CDCl₃) δ_{H} 5.41 (m, 1H), 2.45-2.38 (m, 1H), 2.10-1.85 (m, 6H), 1.48-1.23 (m, 13H), 1.22-1.17 (m, 8H), 1.16-0.99 (m, 8H), 0.94-0.87 (m, 10H), 0.68 (s, 3H). LC-ELSD/MS 30-90AB_2 min_E, purity >95%, MS pap ESI calcd. for C₂₉H₅₀₀₂ [M−2H₂O+H]⁺ 395.4, found 395.4.

### Synthesis of 1-9a

To a suspension of D-9.6a (166 mg, 0.293 mmol) in THF (3 mL) was added TBAF (0.586 mL, 1M in THF) at 25° C. and the mixture was stirred at 80° C. for 16 hours. The mixture was quenched with saturated NH₄Cl (5 mL) and extracted with EtOAc (2×30 mL). The combined organic phase was washed with water (3×30 mL), dried over Na₂SO₄, filtered, and concentrated. Purification by flash column chromatography (0˜30% of EtO Ac in PE) followed by lyophilization provided D-9a (54.2 mg, 42%). ¹H NMR (400 MHz, CDCl₃) δ_{H} 5.50 (m, 1H), 2.51-2.48 (m, 1H), 2.36-2.29 (m, 1H), 2.10-1.83 (m, 5H), 1.70-1.58 (m, 4H), 1.52-1.21 (m, 11H), 1.21-1.19 (m, 6H), 1.18-0.96 (m, 9H), 0.96-0.89 (m, 9H), 0.69 (s, 3H). LC-ELSD/MS 30-90AB_2 min_E, purity >95%, MS ESI calcd. for C₂₉H₅₀₀₂ [M−2H₂O+H]⁺ 395.4, found 395.4.

### Example 53: (1R,3aS,3bR,5aS,8R,10aS,10bS,12aR)-8-ethyl-10a,12a-dimethyl-1-((2R,5R)-6,6,6-trifluoro-5-hydroxy-5-methylhexan-2-yl)octadecahydrocyclohepta[a]cyclopenta[f]naphthalen-8-ol (D-10); (1R,3aS,3bR,5aS,8S,10aS,10bS,12aR)-8-ethyl-10a,12a-dimethyl-1-((2R,5R)-6,6,6-trifluoro-5-hydroxy-5-methylhexan-2-yl)octadecahydrocyclohepta[a]cyclopenta[f]naphthalen-8-ol (D-11); (1R,3aS,3bR,5aS,8R,10aS,10bS,12aR)-8-ethyl-10a,12a-dimethyl-1-((2R,5S)-6,6,6-trifluoro-5-hydroxy-5-methylhexan-2-yl)octadecahydrocyclohepta[a]cyclopenta[f]naphthalen-8-ol (D-12); (1R,3aS,3bR,5aS,8S,10aS,10bS,12aR)-8-ethyl-10a,12a-dimethyl-1-((2R,5S)-6,6,6-trifluoro-5-hydroxy-5-methylhexan-2-yl)octadecahydrocyclohepta[a]cyclopenta[f]naphthalen-8-ol (D-13); (1R,3aS,3bS,7R,10aR,10bS,12aR)-7,10a,12a-trimethyl-1-((2R,5S)-6,6,6-trifluoro-5-hydroxy-5-methylhexan-2-yl)-1,2,3,3a,3b,4,6,7,8,9,10,10a, 10b, 11,12,12a-hexadecahydrocyclohepta[a]cyclopenta[f]naphthalen-7-ol (D-14); (1R,3aS,3bS,7S,10aR,10bS,12aR)-7,10a,12a-trimethyl-1-((2R,5S)-6,6,6-trifluoro-5-hydroxy-5-methylhexan-2-yl)-1,2,3,3a,3b,4,6,7,8,9,10,10a,10b,11,12,12a-hexadecahydrocyclohepta[a]cyclopenta[f]naphthalen-7-ol (D-15); (1R,3aS,3bS,7R,10aR,10bS,12aR)-7-ethyl-10a,12a-dimethyl-1-((2R,5S)-6,6,6-trifluoro-5-hydroxy-5-methylhexan-2-yl)-1,2,3,3a,3b,4,6,7,8,9,10,10a,10b,11,12,12a-hexadecahydrocyclohepta[a]cyclopenta[f]naphthalen-7-ol (D-16); (1R,3aS,3bS,7S,10aR,10bS,12aR)-7-ethyl-10a,12a-dimethyl-1-((2R,5S)-6,6,6-trifluoro-5-hydroxy-5-methylhexan-2-yl)-1,2,3,3a,3b,4,6,7,8,9,10,10a,10b,11,12,12a-hexadecahydrocyclohepta[a]cyclopenta[f]naphthalen-7-ol (D-17)

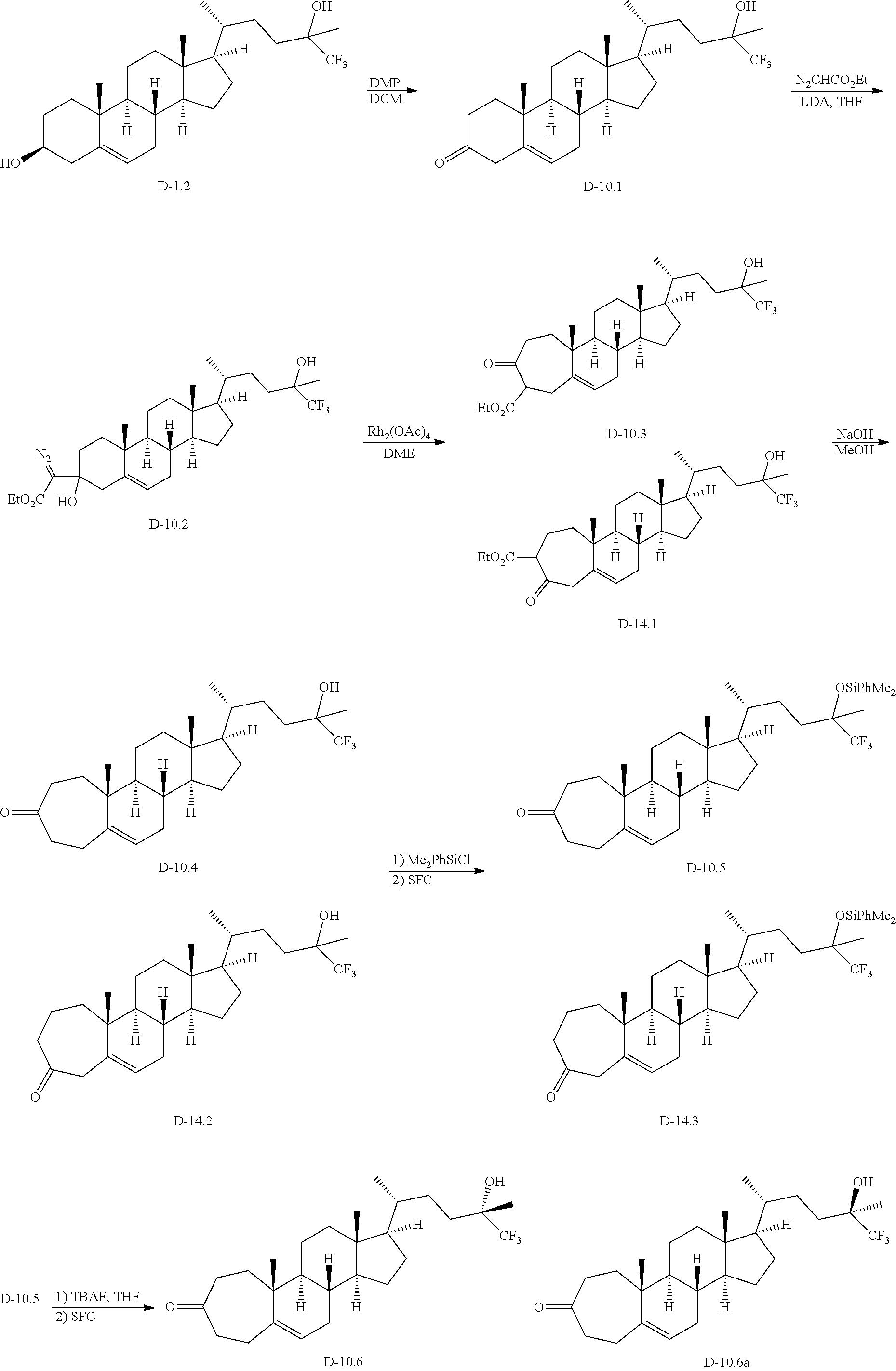

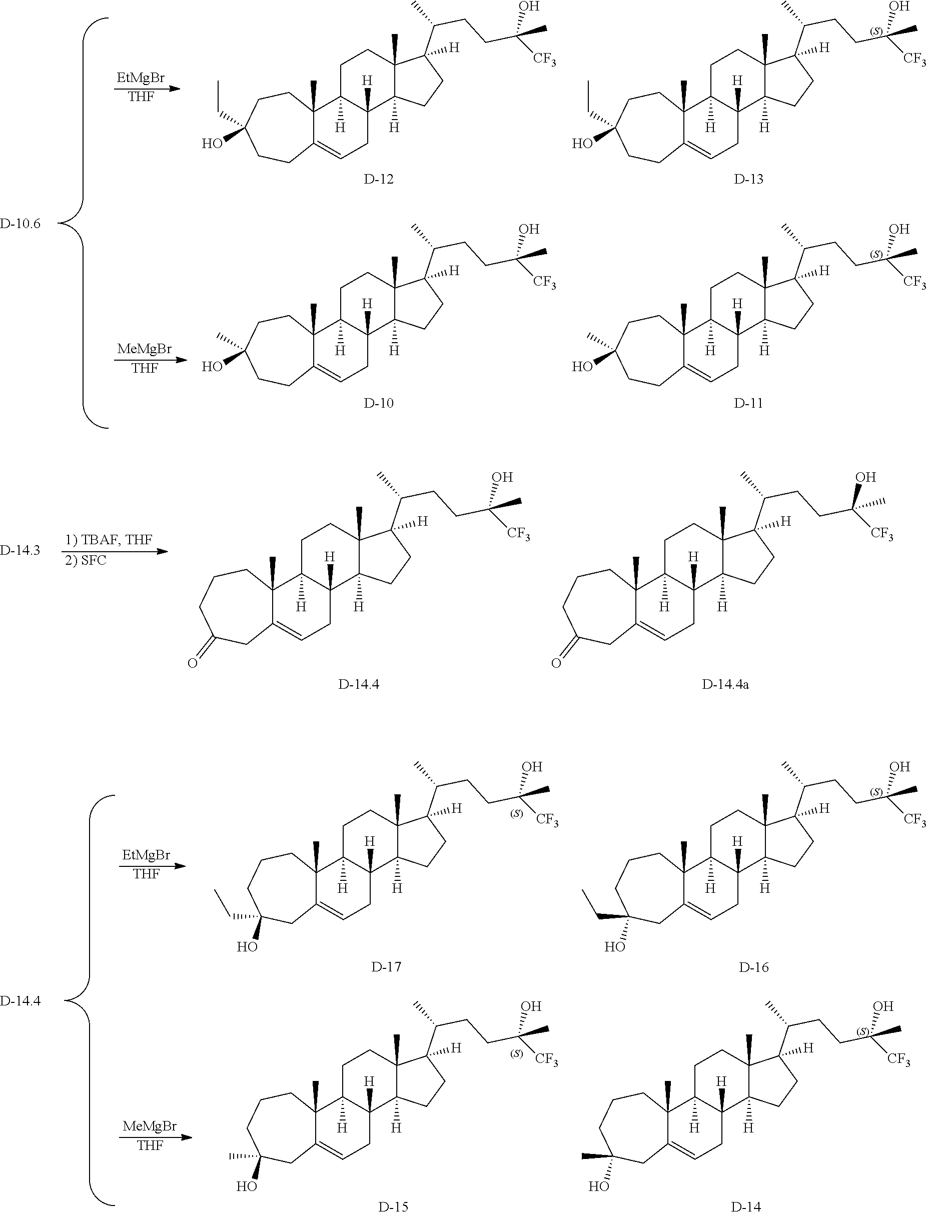

### Synthesis of D-10.1

To a solution of D-1.2 (20.0 g, 45.1 mmol) in DCM (300 mL) was added DMP (57.2 g, 135 mmol) and the mixture was stirred at 20° C. for 3 hours. The mixture was quenched with saturated NaHCO₃/Na₂S₂O₃ (1:1, 2×500 mL). The DCM phase was separated and washed with brine (500 mL), dried over Na₂SO₄, filtered, and concentrated to give D-10.1 (25.0 g).

### Synthesis of D-10.2

To a mixture of D-10.1 (25.0 g, 56.7 mmol) and ethyl diazoacetate (32.2 g, 283 mmol) in THF (800 mL) at -70° C. was added LDA (141 mL, 283 mmol, 2M). The mixture was stirred at −70° C. for 4 hours and then acetic acid (16.9 g, 283 mmol) in THF (20 mL) was added. The mixture was warmed to 25° C. and stirred for 16 hours. Water (500 mL) and PE (300 mL) were added, the organic phase was separated, and the aqueous phase was extracted with EtOAc (500 mL). The combined organic layers were washed with saturated brine (500 mL), dried over anhydrous Na₂SO₄, filtered, and concentrated to give D-10.2 (41.0 g) which was used directly in next step.

### Synthesis of D-10.3 and D-14.1

To a solution of D-10.2 (41.0 g, 73.9 mmol) in DME (500 mL) was added Rh₂(OAc)₄ (649 mg, 1.47 mmol) and the mixture was stirred at 25° C. for 16 hours. The reaction mixture was concentrated to give a mixture of D-10.3 and D-14.1 (43.0 g) which was used directly in next step.

### Synthesis of D-10.4 and D-14.2

To a mixture of D-10.3 and D-14.1 (43.0 g, 81.6 mmol) in MeOH (500 mL) was added H₂O (150 mL) and NaOH (32.5 g, 815 mmol) and the mixture was stirred at 60° C. for 16 hours. The reaction mixture was concentrated, H₂O (200 mL) was added, and the mixture was extracted with EtOAc (3×300 mL). The combined organic phase was washed with saturated brine (150 mL), dried over anhydrous Na₂SO₄, filtered, and concentrated. The residue was purified by silica gel chromatography (0-30% of EtOAc in PE) to give a mixture of D-10.4 and D-14.2 (3.40 g).

### Synthesis of D-10.5 and D-14.3

To a solution of D-10.4 and D-14.2 (3.40 g, 7.47 mol) in DCM (50 ml) was added imidazole (1.01 g, 14.9 mmol), DIPEA (1.92 g, 14.9 mmol), chlorodimethylphenylsilane (3.82 g, 22.4 mmol) and the reaction mixture was stirred at 20° C. for 16 hours. The mixture was poured into NaHCO₃ (100 mL) and extracted with DCM (3×50 mL). The combined organic phase was washed with saturated brine (100 mL), dried over anhydrous Na₂SO₄, filtered, and concentrated. The residue was purified by flash column chromatography (0˜10% of EtOAc in PE) and further separated by SFC (Column: DAICEL CHIRALCEL OD (250 mm*50 mm, 10 um); Condition: 0.1% NH₃H₂O EtOH; Begin B: 20%; End B: 20%; FlowRate (mL/min): 200; Injections: 180) to provide D-14.3 (1.00 g) and D-10.5 (1.00 g).

D-10.5: ¹H NMR (400 MHz, CDCl₃) δ_{H} 7.66-7.56 (m, 2H), 7.45-7.32 (m, 3H), 5.63-5.55 (m, 1H), 2.72-1.98 (m, 8H), 1.89-1.64 (m, 4H), 1.57-1.04 (m, 18H), 1.02 (s, 3H), 0.91 (d, J=6.8 Hz 3H), 0.70 (s, 3H), 0.48-0.40 (m, 6H).

D-14.3: ¹H NMR (400 MHz, CDCl₃) δ_{H} 7.65-7.52 (m, 2H), 7.43-7.32 (m, 3H), 5.60-5.52 (m, 1H), 3.25 (d, J=14.8 Hz, 1H), 2.82 (d, J=14.4 Hz, 1H), 2.64-2.53 (m, 1H), 2.27-2.15 (m, 1H), 2.12-1.97 (m, 2H), 1.91-1.58 (m, 6H), 1.54-1.02 (m, 18H), 0.98 (s, 3H), 0.89 (d, J=6.4 Hz, 3H), 0.68 (s, 3H), 0.44-0.40 (m, 6H).

### Synthesis of D-10.6 and D-10.6a

To a solution of D-10.5 (1.00 g, 1.69 mmol) in THF (10 mL) under N₂ was added TBAF (8.45 mL, 8.45 mmol) and the mixture was stirred at 25° C. for 16 hours. Water (20 mL) was added and the mixture was extracted with EtOAc (2×50 mL). The organic layers were combined, washed with brine (40 mL), dried over Na₂SO₄, filtered, and concentrated. The residue was purified by flash column chromatography (0˜20% of EtOAc in PE) and further purified by SFC (Column: DAICEL CHIRALCEL AD (250 mm*30 mm, 10 um); Condition: 0.1% NH₃H₂O ETOH; Begin B: 20%; End B: 20%; FlowRate (mL/min): 60; Injections: 140) to provide D-10.6 (220 mg) and D-10.6a (230 mg). D-10.6 (10.0 mg, 0.02199 mmol) was lyophilized to give D-10.6 (6.60 mg, 66.3%). D-10.6a (20.0 mg, 0.04399 mmol) was lyophilized to give D-10.6a (16.6 mg, 83.4%).

D-10.6: ¹H NMR (400 MHz, CDCl₃) δ_{H} 5.60-5.54 (m, 1H), 2.66-2.57 (m, 1H), 2.50-2.26 (m, 4H), 2.20-2.12 (m, 1H), 2.07-1.98 (m, 2H), 1.90-1.82 (m, 1H), 1.75-1.67 (m, 3H), 1.62-1.41 (m, 10H), 1.32 (s, 3H), 1.21-1.05 (m, 6H), 1.00 (s, 3H), 0.94 (d, J=6.4 Hz, 3H), 0.70 (s, 3H). ¹⁹F NMR (376 MHz, CDCl₃) δ_{F} −83.13 (s). LC-ELSD/MS 30-90AB_2 min_E, purity>99%, MS ESI calcd. for C₂₇H₄₂F₃O₂ [M+H]⁺ 455.3, found 455.3.

D-10.6a: ¹H NMR (400 MHz, CDCl₃) δ_{H} 5.61-5.52 (m, 1H), 2.65-2.57 (m, 1H), 2.49-2.28 (m, 4H), 2.20-2.12 (m, 1H), 2.06-1.98 (m, 2H), 1.89-1.71 (m, 4H), 1.63-1.45 (m, 10H), 1.33 (s, 3H), 1.27-1.08 (m, 6H), 1.00 (s, 3H), 0.95 (d, J=6.4 Hz, 3H), 0.70 (s, 3H). ¹⁹F NMR (376 MHz, CDCl₃) δ_{F} −82.81 (s). LC-ELSD/MS 30-90AB_2 min_E, purity>99%, MS ESI calcd. for C₂₇H₄₂F₃O₂ [M−H₂O+H]⁺ 437.3, found 437.3.

### Synthesis of D-10 and D-11

A solution of MeMgBr (1 mL, 3 mmol, 3 M) was reacted with D-10.6 (100 mg, 0.2199 mmol) in THF (2 mL) at 25° C. under N₂ and the mixture was stirred for 30 minutes. NH₄Cl (10 mL) was added and the mixture stirred for 5 minutes then extracted with EtOAc (3×20 mL). The combined organic phase was washed with saturated brine (2×20 mL), dried over anhydrous Na₂SO₄, filtered, and concentrated. The residue was purified by flash column chromatography (0˜20% of EtOAc in PE) to give D-10 (50.0 mg, 48.5%) and D-11 (15.0 mg, 14.5%). D-10 (50.0 mg, 0.1062 mmol) was lyophilized to provide D-10 (42.4 mg, 85.1%). D-11 (15.0 mg, 0.03187 mmol) was lyophilized to provide D-11 (9.80 mg, 65.7%).

D-10: ¹H NMR (400 MHz, CDCl₃) δ_{H} 5.40 (br d, J=4.0 Hz, 1H), 2.28-2.19 (m, 1H), 2.06-1.95 (m, 2H), 1.90-1.59 (m, 9H), 1.50-1.23 (m, 13H), 1.20-0.98 (m, 10H), 0.94 (d, J=6.4 Hz, 3H), 0.91 (s, 3H), 0.69 (s, 3H). ¹⁹F NMR (376 MHz, CDCl₃) δ_{F} −83.12 (s). LC-ELSD/MS 30-90AB_2 min_E, purity>99%, MS ESI calcd. for C28H₄₅F₃O₂ [M−H₂O+H]⁺ 453.3, found 453.3.

D-11: ¹H NMR (400 MHz, CDCl₃) δ_{H} 5.45 (br d, J=4.0 Hz, 1H), 2.07-1.59 (m, 12H), 1.50-1.36 (m, 6H), 1.34-0.99 (m, 17H), 0.94 (d, J=6.4 Hz, 3H), 0.88 (s, 3H), 0.69 (s, 3H). ¹⁹F NMR (376 MHz, CDCl₃) δ_{F} −83.14 (s). LC-ELSD/MS 30-90AB_2 min_E, purity>99%, MS ESI calcd. for C28H₄₅F₃O₂ [M−H₂O+H]⁺ 453.3, found 453.3.

### Synthesis of D-12 and D-13

A solution of EtMgBr (1 mL, 3 mmol, 3 M) was reacted with D-10.6 (100 mg, 0.2199 mmol) in THF (2 mL) at 25° C. under N₂ and the mixture was stirred for 30 minutes. NH₄Cl (10 mL) was added and the mixture was stirred for 5 minutes. The aqueous phase was extracted with EtOAc (3×20 mL) and the combined organic phase was washed with saturated brine (2×20 mL), dried over anhydrous Na₂SO₄, filtered, and concentrated. The residue was purified by flash column chromatography (0˜20% of EtOAc in PE) to give D-12 (50.0 mg, 47.1%) and D-13 (15.0 mg, 14.1%). D-12 (50.0 mg, 0.1031 mmol) was lyophilized to give D-12 (42.1 mg, 84.3%). D-13 (15.0 mg, 0.03094 mmol) was lyophilized to give D-13 (8.20 mg, 55%).

D-12: ¹H NMR (400 MHz, CDCl₃) δ_{H} 5.40 (br d, J=4.0 Hz, 1H), 2.29-2.21 (m, 1H), 2.08-1.58 (m, 12H), 1.49-1.24 (m, 14H), 1.21-1.01 (m, 7H), 0.94 (d, J=6.8 Hz, 3H), 0.91 (s, 3H), 0.87 (t, J=7.6 Hz, 3H), 0.69 (s, 3H). ¹⁹F NMR (376 MHz, CDCl₃) δ_{F} −83.12 (s). LC-ELSD/MS 30-90AB_2 min_E, purity>99%, MS ESI calcd. for C₂₉H₄₇F₃O₂ [M−H₂O+H]⁺ 467.3, found 467.3.

D-13: ¹H NMR (400 MHz, CDCl₃) δ_{H} 5.45 (br d, J=4.3 Hz, 1H), 2.06-1.61 (m, 12H), 1.51-1.24 (m, 14H), 1.21-1.00 (m, 8H), 0.97-0.86 (m, 9H), 0.69 (s, 3H). ¹⁹F NMR (376 MHz, CDCl₃) δ_{F} −83.14 (s). LC-ELSD/MS 30-90AB_2 min_E, purity>99%, MS ESI calcd. for C₂₉H₄₇F₃O₂ [M−H₂O+H]⁺ 467.3, found 467.3.

### Synthesis of D-14.4 and D-14.4a

To a solution of D-14.3 (1.00 g, 1.69 mmol) in THF (10 mL) was added TBAF (8.45 mL, 8.45 mmol) under N₂ and the mixture was stirred at 25° C. for 16 hours. Water (20 mL) was added and the mixture was extracted with EtOAc (2×50 mL), washed with brine (40 mL), dried over Na₂SO₄, filtered, and concentrated. The residue was purified by flash column chromatography (0˜20% of EtOAc in PE) to give D-14.4 and D-14.4a (560 mg, 72.9%). The mixture of D-14.4 and D-14.4a was further purified by SFC (Column: DAICEL CHIRALCEL AD (250 mm*30 mm, 10 um); Condition: 0.1% NH₃H₂O ETOH; Begin B: 15%; End B: 15%; FlowRate (mL/min): 70; Injections: 70) twice to give D-14.4 (180 mg, 32.1%) and D-14.4a (270 mg, 48.2%). D-14.4 (10.0 mg, 0.02199 mmol) was lyophilized to give D-14.4 (6.60 mg, 66.3%). D-14.4a (20.0 mg, 0.04399 mmol) was lyophilized to get D-14.4a (16.6 mg, 83.4%).

D-14.4: ¹H NMR (400 MHz, CDCl₃) δ_{H} 5.59-5.53 (m, 1H), 3.25 (d, J=14.0 Hz, 1H), 2.82 (d, J=14.8 Hz, 1H), 2.65-2.55 (m, 1H), 2.27-2.15 (m, 1H), 2.12-1.98 (m, 2H), 1.91-1.80 (m, 2H), 1.72-1.44 (m, 14H), 1.32 (s, 3H), 1.26-1.07 (m, 6H), 0.99 (s, 3H), 0.94 (d, J=6.8 Hz, 3H), 0.70 (s, 3H). ¹⁹F NMR (376 MHz, CDCl₃) δ_{F} −83.13 (s). LC-ELSD/MS 30-90AB_2 min_E, purity>99%, MS ESI calcd. for C₂₇H₄₁F₃O₂ [M+H]⁺ 455.3, found 455.3.

D-14.4a: ¹H NMR (400 MHz, CDCl₃) δ_{H} 5.59-5.53 (m, 1H), 3.25 (d, J=14.4 Hz, 1H), 2.82 (d, J=14.4 Hz, 1H), 2.65-2.55 (m, 1H), 2.25-2.16 (m, 1H), 2.10-1.98 (m, 2H), 1.91-1.69 (m, 4H), 1.63-1.43 (m, 11H), 1.33 (s, 3H), 1.30-1.06 (m, 7H), 0.98 (s, 3H), 0.95 (d, J=6.8 Hz, 3H), 0.70 (s, 3H). ¹⁹F NMR (376 MHz, CDCl₃) δ_{F} −82.83 (s). LC-ELSD/MS 30-90AB_2 min_E, purity>99%, MS ESI calcd. for C₂₇H₄₁F₃O₂ [M+H]⁺ 455.3, found 455.3.

### Synthesis of D-14 and D-15

A solution of MeMgBr (1 mL, 3 mmol, 3 M) was reacted with D-14.4 (85.0 mg, 0.1869 mmol) in THF (2 mL) at 25° C. under N₂ and the mixture was stirred for 1 hour. NH₄Cl (10 mL) was added and the mixture was stirred for 5 minutes. The mixture was extracted with EtOAc (3×20 mL) and the combined organic phase was washed with saturated brine (2×20 mL), dried over anhydrous Na₂SO₄, filtered, and concentrated. The residue was purified by flash column chromatography (0˜20% of EtOAc in PE) to give D-14 (40.0 mg, 45.7%) and D-15 (20.0 mg, 22.8%). D-15 (20.0 mg, 0.04249 mmol) was lyophilized to give D-15 (18.6 mg, 93.4%). D-14 (40.0 mg, 0.08494 mmol) was lyophilized to give D-14 (38.5 mg, 96.1%).

D-14: ¹H NMR (400 MHz, CDCl₃) δ_{H} 5.56-5.47 (m, 1H), 2.41 (br d, J=13.2 Hz, 1H), 2.14-1.67 (m, 11H), 1.50-1.27 (m, 11H), 1.24-1.03 (m, 12H), 0.96-0.92 (m, 6H), 0.69 (s, 3H). ¹⁹F NMR (376 MHz, CDCl₃) δ_{F} −83.14 (s). LC-ELSD/MS 30-90AB_2 min_E, purity>99%, MS ESI calcd. for C28H₄₅F₃O₂ [M−H₂O+H]⁺ 453.3, found 453.3.

D-15: ¹H NMR (400 MHz, CDCl₃) δ_{H} 5.46-5.39 (m, 1H), 2.46 (d, J=12.8 Hz, 1H), 2.09-1.61 (m, 10H), 1.52-1.27 (m, 12H), 1.26-1.03 (m, 12H), 0.94 (d, J=6.8 Hz, 3H), 0.91 (s, 3H), 0.69 (s, 3H). ¹⁹F NMR (376 MHz, CDCl₃) δ_{F} −83.13 (s). LC-ELSD/MS 30-90AB_2 min_E, purity >99%, MS ESI calcd. for C28H₄₅F₃O₂ [M−H₂O+H]⁺ 453.3, found 453.3.

### Synthesis of D-16 and D-17

A solution of EtMgBr (1 mL, 3 mmol, 3 M) was reacted with D-14.4 (85.0 mg, 0.1869 mmol) in THF (2 mL) at 25° C. under N₂ and the mixture was stirred for 1 hour. NH₄Cl (10 mL) was added and the mixture was stirred for 5 minutes. The mixture was extracted with EtOAc (3×20 mL) and the combined organic phase was washed with saturated brine (2×20 mL), dried over anhydrous Na₂SO₄, filtered, and concentrated. The residue was purified by flash column chromatography (0˜20% of EtOAc in PE) to give D-16 (40.0 mg, 44.3%) and D-17 (30.0 mg, 33.2%). D-17 (30.0 mg, 0.06189 mmol) was lyophilized to give D-17 (27.3 mg, 91.3%). D-16 (15.0 mg, 0.03094 mmol) was lyophilized to give D-16 (8.20 mg, 55%).

D-16: ¹H NMR (400 MHz, CDCl₃) δ_{H} 5.54-5.47 (m, 1H), 2.33 (d, J=12.8 Hz, 1H), 2.11-1.63 (m, 11H), 1.54-1.34 (m, 7H), 1.33-0.99 (m, 15H), 0.95-0.90 (m, 9H), 0.69 (s, 3H). ¹⁹F NMR (376 MHz, CDCl₃) δ_{F} −83.14 (s). LC-ELSD/MS 30-90AB_2 min_E, purity>99%, MS ESI calcd. for C₂₉H₄₇F₃O₂ [M−H₂O+H]⁺ 467.4, found 467.3.

D-17: ¹H NMR (400 MHz, CDCl₃) δ_{H} 5.43-5.38 (m, 1H), 2.41 (d, J=12.4 Hz, 1H), 2.11-1.64 (m, 10H), 1.53-1.19 (m, 16H), 1.18-1.01 (m, 7H), 0.95-0.87 (m, 9H), 0.68 (s, 3H). ¹⁹F NMR (376 MHz, CDCl₃) δ_{F} −83.12 (s). LC-ELSD/MS 30-90AB_2 min_E, purity>99%, MS ESI calcd. for C₂₉H₄₇F₃O₂ [M−H₂O+H]⁺ 467.4, found 467.4.

### Example 54: Synthesis of (3S,5S,8S,10S,13R,14S,17R)-3-ethyl-17-((R)-6-hydroxy-6-methylheptan-2-yl)-10,13-dimethyl-2,3,4,5,6,7,8,10,12,13,14,15,16,17-tetradecahydro-1H-cyclopenta[a]phenanthren-3-ol (E-1)

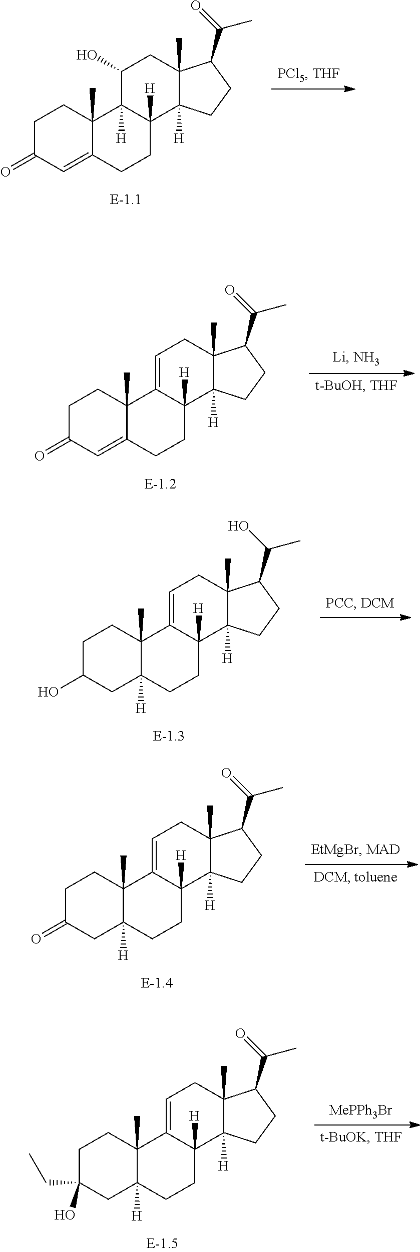

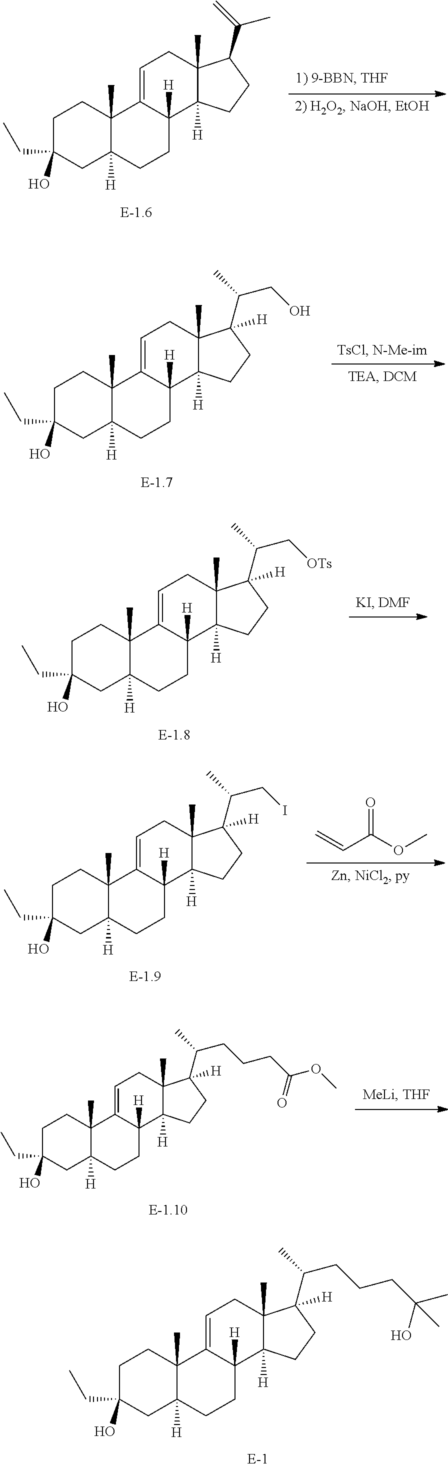

### Synthesis of E-1.2

To a solution of E-1.1 (50.0 g, 151 mmol) in THF (1.5 L) was added PCl₅ (62.8 g, 302 mmol) at −70° C. under N₂ and the mixture was stirred 1 hour. The mixture was quenched by NaHCO₃ (1.5 L, sat.) and extracted with EtOAc (1.5 L). The organic layer was separated, dried over Na₂SO₄, filtered, and concentrated to give E-1.2 (60.0 g). ¹H NMR (400 MHz, CDCl₃) δ_{H} 5.77-5.73 (m, 1H), 5.55-5.49 (m, 1H), 2.63-2.45 (m, 4H), 2.38-2.18 (m, 5H), 2.15-1.71 (m, 9H), 1.45-1.32 (m, 5H), 1.18-1.06 (m, 1H), 0.61 (s, 3H).

### Synthesis of E-1.3

Lithium (5.20 g, 750 mmol) was added to liquid ammonia (750 mL) in portions at 70° C. After stirring at −70° C. for 1 hour, a solution of E-1.2 (47.0 g, 150 mmol) in dry THF (750 mL) and t-butanol (44.4 g) were added to this mixture and the temperature was maintained below −60° C. The mixture was stirred at −70° C. for 1 hour then ammonium chloride (53.4 g) was added and the mixture was warmed to room temperature and stirred for 16 hours. The reaction mixture was diluted with H₂O (500 mL) and THF (500 mL) and then filtered and extracted with DCM (500 ml). The organics were concentrated under vacuum to give E-1.3 (54.0 g).

### Synthesis of E-1.4

To E-1.3 (54.0 g, 169 mmol) in DCM (750 mL) was added silica gel (54.5 g) and PCC (54.5 g, 253 mmol) at 0° C. and the mixture was stirred at 25° C. for 1 hour. PE (400 mL) was added and the mixture was filtered, washed with DCM (2×100 mL), and the organics were concentrated. The residue was purified by column chromatography (15˜35% of EtOAc in PE) to give the E-1.4 (31.0 g, 58%). ¹H NMR (400 MHz, CDCl₃) δ_{H} 5.43-5.35 (m, 1H), 2.64-2.17 (m, 8H), 2.10-1.59 (m, 9H), 1.51-1.26 (m, 5H), 1.15 (s, 3H), 1.06-0.94 (m, 1H), 0.58 (s, 3H).

### Synthesis of E-1.5

To a solution of BHT (130 g, 590 mmol) in toluene (260 mL) under nitrogen at 0° C. was added AlMe₃ (2 M in toluene, 147 mL, 295 mmol) dropwise. The mixture was stirred at 25° C. for 1 hour and E-1.4 (31.0 g, 98.5 mmol) in anhydrous DCM was added (150 mL) dropwise at −70° C. After stirring at −70° C. for 1 hour under N₂, EtMgBr (98.3 mL, 295 mmol, 3M in ethyl ether) was added drop wise at −70° C. The resulting solution was stirred at −70° C. for another 3 hours. The reaction mixture was poured into citric acid (600 mL, 20% aq.) at below 10° C. and extracted with EtOAc (2×500 mL). The combined organic layer was dried over Na₂SO₄, filtered, and concentrated to give the product which was purified by column (20˜30% EtOAc in PE) to give E-1.5 (28.0 g, 83%). ¹H NMR (400 MHz, CDCl₃) δ_{H} 5.42-5.33 (m, 1H), 2.61-2.12 (m, 6H), 2.05-1.54 (m, 10H), 1.43-1.14 (m, 9H), 0.98-0.85 (m, 7H), 0.54 (s, 3H).

### Synthesis of E-1.6

To MePPh₃Br (27.9 g, 78.3 mmol) in THF (300 mL) was added t-BuOK (8.78 g, 78.3 mmol) at 25° C. under N₂ and the resulting mixture was stirred at 45° C. for 30 mins. E-1.5 (18.0 g, 52.2 mmol) was added in portions below 55° C. The reaction mixture was stirred at 55° C. for 1.5 hours and then quenched with 10% NH₄Cl aqueous (500 mL) at 25° C. The aqueous was extracted with EtOAc (2×500 mL). The combined organic phase was separated, dried over Na₂SO₄, filtered, concentrated, triturated with MeOH/H₂O (1:1, 600 mL) and purified by flash column (0˜25% of EtOAc in PE) to give E-1.6 (12.6 g, 71%). 1H NMR (400 MHz, CDCl₃) δ_{H} 5.31-5.24 (m, 1H), 4.84 (s, 1H), 4.71 (s, 1H), 2.00-1.65 (m, 11H), 1.48-1.13 (m, 13H), 1.03-0.81 (m, 8H), 0.50 (s, 3H).

### Synthesis of E-1.7

To a solution of E-1.6 (1.00 g, 2.91 mmol) in THF (5 mL) was added 9-BBN (29 mL, 0.5 M, 14.5 mmol) at 0° C. under N₂. The mixture was stirred at 25° C. for 16 hours. To the mixture was added ethanol (3.43 ml, 58.2 mmol), NaOH (8.72 mL, 5M aq., 43.6 mmol) and H₂O₂ (4.36 mL, 10 M, 43.6 mmol) dropwise. The mixture was stirred at 25° C. for 1 hour and then quenched by Na₂SO₃ (10 mL, sat. aq.). The mixture was concentrated to remove most organic solvent and filtered. The solid was washed with water (10 mL), dried under vacuum and purified by column (0˜15% EA in PE) to give E-1.7 (600 mg, 58%). ¹H NMR (400 MHz, CDCl₃) δ_{H} 5.28-5.21 (m, 1H), 3.68-3.59 (m, 1H), 3.42-3.35 (m, 1H), 2.20-2.11 (m, 1H), 2.03-1.64 (m, 8H), 1.43-1.15 (m, 14H), 1.06-0.84 (m, 11H), 0.61 (s, 3H).

### Synthesis of E-1.8

To a solution of E-1.7 (600 mg, 1.66 mmol) in DCM (6 mL) was added N-methylimidazole (203 mg, 2.48 mmol), TEA (642 μL, 4.97 mmol) and TsCl (632 mg, 3.32 mmol). The mixture was stirred at 25° C. for 1 hour then poured into NaHCO₃ (10 mL, saturated). The aqueous phase was extracted with DCM (2×10 mL). The combined organic phase was washed with brine (10 mL), dried over anhydrous Na₂SO₄, filtered, and concentrated to give E-1.8 (820 mg, 96%). ¹H NMR (400 MHz, CDCl₃) δ_{H} 7.78 (d, J=8.4 Hz, 2H), 7.34 (d, J=8.0 Hz, 2H), 5.25-5.18 (m, 1H), 4.01-3.94 (m, 1H), 3.82-3.76 (m, 1H), 2.45 (s, 3H), 2.14-1.64 (m, 11H), 1.43-1.09 (m, 12H), 0.99-0.85 (m, 10H), 0.55 (s, 3H).

### Synthesis of E-1.9

To a solution of E-1.8 (820 mg, 1.59 mmol) in DMF (10 mL) was added KI (1.26 g, 7.63 mmol) at 25° C. The mixture was stirred at 60° C. for 1 hours. The mixture was poured into water (20 mL) and stirred for 20 mins and extracted with EtOAc (3×20 mL). The combined organic phase was washed with brine (2×20 mL), dried over anhydrous Na₂SO₄, filtered, concentrated, and purified by silica gel chromatography (0˜15% EtOAc in PE) to give E-1.9 (350 mg, 47%). ¹H NMR (400 MHz, CDCl₃) δ_{H} 5.27-5.21 (m, 1H), 3.38-3.31 (m, 1H), 3.20-3.12 (m, 1H), 2.14-1.68 (m, 8H), 1.44-1.16 (m, 14H), 1.04-0.85 (m, 11H), 0.63 (s, 3H).

### Synthesis of E-1.10

To a mixture of Zn powder (339 mg, 5.20 mmol) in dry pyridine (8 mL) was added slowly methyl acrylate (447 mg, 5.20 mmol) and NiCl₂ (143 mg, 1.11 mmol) at 20° C. under N₂. After stirring at 60° C. for 2 hours the mixture was cooled to 20° C. and a solution of E-1.9 (350 mg, 0.7439 mmol) was added slowly. The reaction mixture was stirred for 1.5 hours at 20° C. for another 2 hours then diluted with NH₄Cl (20 mL) and filtered through a pad of silica gel. The filtrate was extracted with EtOAc (2×20 mL). The organic phase was dried over Na₂SO₄, filtered, purified by flash column (0˜20% of EtOAc in PE) and purified from MeCN (45 mL) to give E-1.10 (200 mg, 63%). ¹H NMR (400 MHz, CDCl₃) δ_{H} 5.25-5.21 (m, 1H), 3.66 (s, 3H), 2.33-2.10 (m, 4H), 2.02-1.64 (m, 6H), 1.47-1.33 (m, 9H), 1.23-1.08 (m, 7H), 0.98-0.81 (m, 13H), 0.58 (s, 3H).

### Synthesis of E-1

To a solution of E-1.10 (100 mg, 0.2321 mmol) in THF (3 ml) was added methyllithium (1.45 mL, 2.32 mmol, 1.6 M in Et₂O) dropwise at 0° C. under N₂ and the mixture was stirred at 25° C. for 2 hours. The reaction mixture was quenched by saturated NH₄Cl (10 mL) and extracted with ethyl acetate (3×10 mL). The combined organic phase was washed with brine (2×10 mL), dried over anhydrous Na₂SO₄, filtered, and concentrated. Purification by column chromatography (0˜25% of EtOAc in PE) followed by lyophilization provided E-1 (61.4 mg, 61%). ¹H NMR (400 MHz, CDCl₃) δ_{H} 5.28-5.17 (m, 1H), 2.24-2.09 (m, 1H), 2.02-1.61 (m, 8H), 1.47-1.34 (m, 11H), 1.05-1.40 (m, 15H), 0.98-0.85 (m, 11H), 0.59 (s, 3H). LC-ELSD/MS purity >99%; MS ESI calcd for C₂₉H₅₀₀₂ [M−2H₂O+H]⁺ 395.4, found 395.4.

### Example 55: Synthesis of (3S,5S,8S,10S,13R,14S,17R)-3-ethyl-10,13-dimethyl-17-((2R,5R)-6,6,6-trifluoro-5-hydroxy-5-methylhexan-2-yl)-2,3,4,5,6,7,8,10,12,13,14,15,16,17-tetradecahydro-1H-cyclopenta[a]phenanthren-3-ol (E-2)

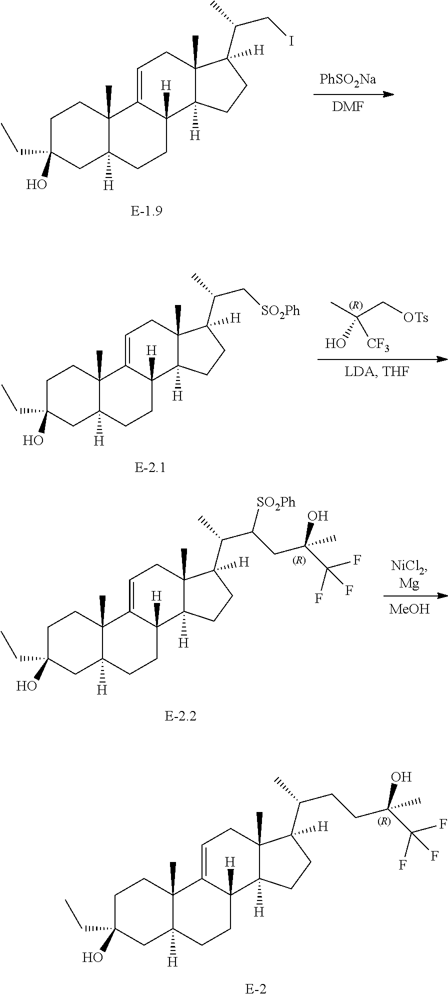

### Synthesis of E-2.1

To a solution of E-1.9 (4.00 g, 8.5 mmol) in DMF (40 mL) was added sodium benzenesulfinate (4.18 g, 25.5 mmol) and the mixture was stirred at 40° C. for 16 hours. The reaction mixture was poured into water (100 mL) and filtered, dried under vacuum, and purified by flash column (15˜25% EtOAc in PE) to give E-2.1 (3.00 g, 73%). ¹HNMR (400 MHz, CDCl₃) δ_{H} 7.95-7.87 (m, 2H), 7.68-7.62 (m, 1H), 7.60-7.52 (m, 2H), 5.25-5.18 (m, 1H), 3.15 (d, J=12.8 Hz, 1H), 2.86 (dd, J=10.0, 14.4 Hz, 1H), 2.18-2.05 (m, 2H), 1.99-1.51 (m, 9H), 1.43-1.05 (m, 15H), 0.96-0.83 (m, 7H), 0.57 (s, 3H).

### Synthesis of E-2.2

To a solution of E-2.1 (500 mg, 1.03 mmol) in THF (8 mL) at −70° C. under N₂ was added dropwise n-BuLi (1.64 mL, 4.12 mmol, 2.5M in hexane). The reaction mixture was stirred at −70° C. for 0.5 hour. DIPA (416 mg, 4.12 mmol) was added dropwise at −70° C. for 0.5 hour. (2R)-1,1,1-trifluoro-2-methyl-3-[(4-methylbenzenesulfonyl)oxy]propan-2-ol (396 mg, 1.33 mmol) was added dropwise at −70° C. The reaction mixture was warmed to 25° C. slowly and stirred at 25° C. for 18 hours. The reaction mixture was quenched with saturated NH₄Cl (10 mL) and extracted with EtOAc (2×10 mL). The combined organic phase was washed with 10% NH₄Cl (10 mL), dried over Na₂SO₄, filtered and concentrated under vacuum to give E-2.2 (560 mg).

### Synthesis of E-2

To a solution of E-2.2 (560 mg, 0.9168 mmol) in 50 mL of dry methanol was added nickel (II) chloride (23.6 mg, 0.1833 mmol) and Mg powder (1.32 g, 55.0 mmol) in four portions under N₂ at 65° C. The reaction mixture was quenched by citric acid (50 mL, 10%) which was added dropwise and extracted with EtOAc (3×50 mL). The organic layer was washed with NaHCO₃ (50 mL), brine (50 mL), dried over Na₂SO₄, filtered, and concentrated. Purification by column chromatography (0˜15% of EtOAc in PE) followed by lyophilization provided E-2 (145.1 mg, 33%). ¹H NMR (400 MHz, CDCl₃) δ_{H} 5.24 (d, J=6.0 Hz, 1H), 2.21-2.09 (m, 1H), 2.00-1.64 (m, 9H), 1.53-1.37 (m, 8H), 1.35-1.09 (m, 12H), 0.99-0.82 (m, 11H), 0.60 (s, 3H). ¹⁹F NMR (377 MHz, CDCl₃) δ_{F} −82.80 (s). LC-ELSD/MS purity >99%; MS ESI calcd for C28H₄₅F₃O₂ [M−H₂O+H]⁺ 453.4, found 453.4.

### Example 56: Synthesis of (3S,5S,8S,10S,13R,14S,17R)-3-ethyl-10,13-dimethyl-17-((2R,5S)-6,6,6-trifluoro-5-hydroxy-5-methylhexan-2-yl)-2,3,4,5,6,7,8,10,12,13,14,15,16,17-tetradecahydro-1H-cyclopenta[a]phenanthren-3-ol (E-3)

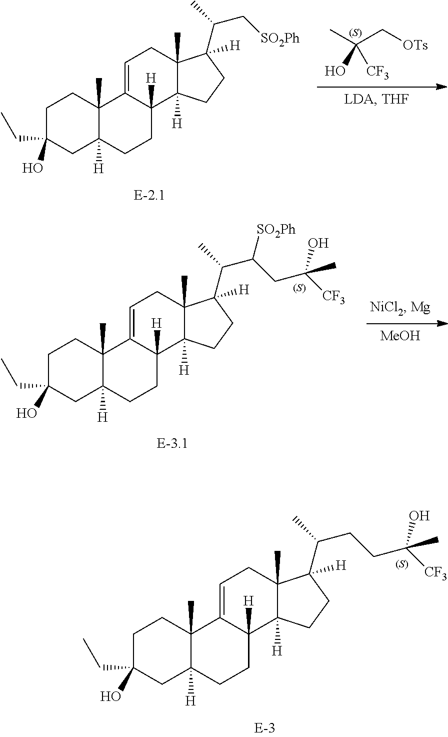

### Synthesis of E-3.1

To a solution of E-2.1 (500 mg, 1.03 mmol) in THF (8 mL) at −70° C. under N₂ at 70° C. was added dropwise n-BuLi (1.64 mL, 4.12 mmol, 2.5M in hexane). The reaction mixture was stirred at -70° C. for 0.5 hour then DIPA (416 mg, 4.12 mmol) was added dropwise for 0.5 hour. (2S)-1,1,1-trifluoro-2-methyl-3-[(4-methylbenzenesulfonyl)oxy]propan-2-ol (396 mg, 1.33 mmol) was added dropwise at −70° C. The reaction mixture was warmed to 25° C. slowly then stirred for 18 hours. The reaction mixture was quenched with saturated NH₄Cl (10 mL) and extracted with EtOAc (2×10 mL). The combined organic phase was washed with 10% NH₄Cl (10 mL), dried over Na₂SO₄, filtered and concentrated to give E-3.1 (610 mg).

### Synthesis of E-3

To a solution of E-3.1 (610 mg, 0.9986 mmol) in 50 mL of dry methanol was added nickel (II) chloride (25.7 mg, 0.1997 mmol) and Mg powder (1.43 g, 59.9 mmol) in four portions under N₂ at 65° C. The reaction mixture was quenched by citric acid (50 mL, 10%) which was added dropwise until solids dissolved. After extraction with EtOAc (3×50 mL), the organic layer was washed with NaHCO₃ (50 mL), brine (50 mL), dried over Na₂SO₄, filtered, and concentrated. Purification by flash column chromatography (0˜15% of EtOAc in PE) and further lyophilization provided E-3 (200.4 mg, 41%). ¹H NMR (400 MHz, CDCl₃) δ_{H} 5.24 (d, J=6.0 Hz, 1H), 2.20-2.10 (m, 1H), 2.01-1.62 (m, 10H), 1.50-1.36 (m, 7H), 1.32 (s, 3H), 1.30-1.00 (m, 9H), 0.96 (s, 3H), 0.95-0.81 (m, 8H), 0.60 (s, 3H). ¹⁹F NMR (377 MHz, CDCl₃) δ_{F} −83.13 (s). LC-ELSD/MS purity >99%; MS ESI calcd. for C₂₈H₄₅F₃O₂ [M−H₂O+H]⁺ 453.3, found 453.3.

### Example 57: Synthesis of (3S,5S,8S,10S,13R,14S,17R)-3-ethyl-17-((2R,5R)-5-hydroxy-6-methylheptan-2-yl)-10,13-dimethyl-2,3,4,5,6,7,8,10,12,13,14,15,16,17-tetradecahydro-1H-cyclopenta[a]phenanthren-3-ol (E-4)

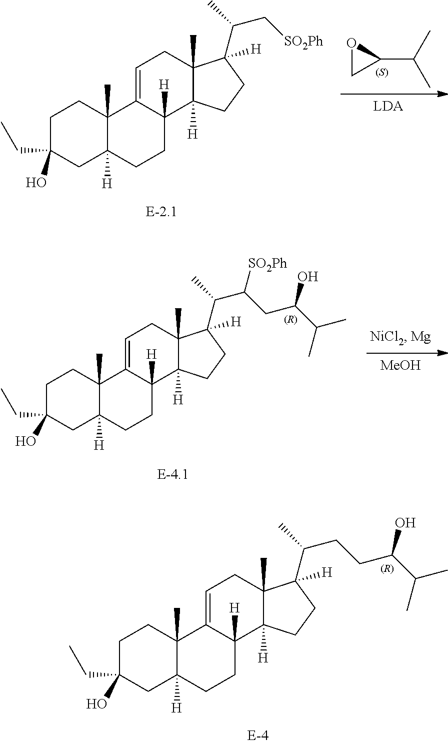

### Synthesis of E-4.1

To a solution of E-2.1 (500 mg, 1.03 mmol) in THF (5 mL) at -70° C. under N₂ at was added dropwise n-BuLi (1.64 mL, 4.12 mmol, 2.5 M in hexane). The reaction mixture was stirred at −70° C. for 0.5 hour. DIPA (416 mg, 4.12 mmol) was added dropwise at −70° C. for 0.5 hour. (2S)-2-(propan-2-yl) oxirane (235 mg, 2.06 mmol, 75%) was added dropwise at 70° C. The reaction mixture was warmed to 25° C. slowly and stirred for 18 hours. The reaction mixture was quenched with saturated NH₄Cl (10 mL) and extracted with EtOAc (2×10 mL). The combined organic phase was washed with brine (10 mL), dried over Na₂SO₄, filtered, and concentrated to give E-4.1 (500 mg).

### Synthesis of E-4

To a solution of E-4.1 (500 mg, 0.88 mmol) in 60 mL of dry methanol was added nickel (II) chloride (22.5 mg, 0.18 mmol) and Mg powder (1.26 g, 52.5 mmol) in six portions under N₂ at 65° C. The reaction mixture was quenched by citric acid (50 mL, 20%) which was added dropwise until solid was dissolved. After extracted with EtOAc (3×50 mL), the organic layer was washed with brine (50 mL), dried over Na₂SO₄, filtered, and concentrated. The residue was purified by flash column (0˜4% of Acetone in DCM) to give E-4 (123.3 mg, 25%). ¹H NMR (400 MHz, CDCl₃) δ_{H} 5.24 (d, J=5.6 Hz, 1H), 3.36-3.27 (m, 1H), 2.22-2.09 (m, 1H), 2.00-1.82 (m, 4H), 1.78-1.62 (m, 4H), 1.47-1.34 (m, 9H), 1.33-1.05 (m, 10H), 0.96 (s, 3H), 0.95-0.79 (m, 14H), 0.59 (s, 3H). LC-ELSD/MS 30-90AB_2 min_E, purity 99%, MS ESI calcd. for C₂₉H₅₀₀₂ [M−H₂O+H]⁺ 413.4, found 413.4.

### Example 58: Synthesis of (3S,5S,8S,10S,13R,14S,17R)-3-ethyl-17-((R)-5-hydroxy-5-methylhexan-2-yl)-10,13-dimethyl-2,3,4,5,6,7,8,10,12,13,14,15,16,17-tetradecahydro-1H-cyclopenta[a]phenanthren-3-ol (E-5)

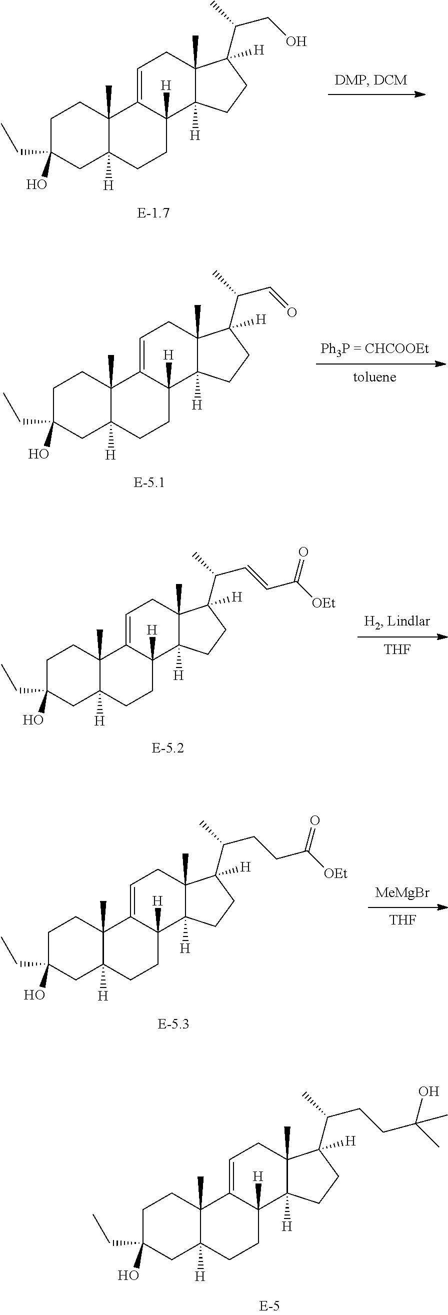

### Synthesis of E-5.1

To a solution of E-1.7 (2.00 g, 5.54 mmol) in DCM (20 mL) was added DMP (3.51 g, 8.30 mmol) at 25° C. and the mixture was stirred for 5 mins. The mixture was added NaHCO₃ (40 mL, sat.) and Na₂S₂O₃ (40 mL, sat.). The aqueous phase was extracted with DCM (2×20 mL) and the combined organic phase was washed with NaHCO₃/Na₂S₂O₃ (1:1, 2×40 mL, sat.), brine (40 mL), dried over anhydrous Na₂SO₄, filtered, and concentrated. The mixture was purified by flash column (15˜30% of EtOAc in PE) to give E-5.1 (1.60 g, 80%). ¹H NMR (400 MHz, CDCl₃) δ_{H} 9.59-9.58 (m, 1H), 5.28-5.23 (m, 1H), 2.39-2.34 (m, 1H), 2.17-2.09 (m, 1H), 2.01-1.81 (m, 5H), 1.45-1.39 (m, 7H), 1.30-1.21 (m, 6H), 1.14-1.11 (m, 3H), 0.97 (s, 3H), 0.90-0.85 (m, 7H), 0.64 (s, 3H)

### Synthesis of E-5.2

To a solution of E-5.1 (1.60 g, 4.46 mmol) in toluene (15 mL), Ph₃P=CHCOOEt (3.10 g, 8.92 mmol) was added and the mixture was stirred at 110° C. for 2 hours. The mixture was poured into saturated NH₄Cl (20 mL) and extracted with EtOAc (2×20 mL). The combined organic phase was washed with saturated brine (20 mL), dried over anhydrous Na₂SO₄, filtered and concentrated in vacuum. The residue was purified by silica gel chromatography (15˜20% EA in PE) to give E-5.2 (1.20 g, 63%). E-5.2 (0.900 g, 2.51 mmol) was further purified by SFC (Column: DAICEL CHIRALPAK AD (250 mm*30 mm, 10 um); Condition: 0.1% NH₃H₂O ETOH; Begin B: 25%; End B: 25%; FlowRate (ml/min): 60; Injections: 70) to give E-5.2 (0.620 g, 69%). ¹H NMR (400 MHz, CDCl₃) δ_{H} 6.91-6.74 (m, 1H), 5.72 (d, J=15.6 Hz, 1H), 5.28-5.21 (m, 1H), 4.17 (q, J=7.2 Hz, 2H), 2.34-1.83 (m, 5H), 1.75-1.53 (m, 9H), 1.44-1.20 (m, 13H), 1.08 (d, J=6.4 Hz, 3H), 0.96 (s, 3H), 0.90-0.84 (m, 3H), 0.63 (s, 3H).

### Synthesis of E-5.3

To a solution of E-5.2 (0.620 g, 1.44 mmol) in THF (20 mL) was added Lindlar catalyst (400 mg). The mixture was stirred at 25° C. under H₂ for 18 hours. The mixture was filtered and concentrated to give E-5.3 (0.600 g, 97%). E-5.3 (20.0 mg, 0.04643 mmol) was lyophilized to give E-5.3 (8.20 mg, 41%). ¹H NMR (400 MHz, CDCl₃) δ_{H} 5.27-5.19 (m, 1H), 4.12 (q, J=6.8 Hz, 2H), 2.42-2.07 (m, 3H), 2.01-1.55 (m, 11H), 1.54-1.06 (m, 17H), 0.96 (s, 3H), 0.93-0.85 (m, 6H), 0.59 (s, 3H). LC-ELSD/MS purity >99%; MS ESI calcd. for C28H₄₆₀₃ [M+H−H₂O]⁺ 413.3, found 413.3.

### Synthesis of E-5

A solution of MeMgBr (1.16 mL, 3.48 mmol, 3M in ethyl ether) was reacted with E-5.3 (100 mg, 0.2321 mmol) in THF (5 mL) at 0° C. The resulting mixture was stirred at 25° C. for another 2 hours. The reaction mixture was poured into saturated aqueous citric acid (50 mL) and extracted with EtOAc (2×50 mL). The combined organic layer was dried over Na₂SO₄, filtered, and concentrated. The residue was purified by flash column (0˜30% of EtOAc in PE) and then lyophilized to give E-5 (64.2 mg, 92%). ¹H NMR (400 MHz, CDCl₃) δ_{H} 5.26-5.21 (m, 1H), 2.19-1.55 (m, 14H), 1.52-1.21 (m, 12H), 1.20-1.13 (m, 9H), 0.96 (s, 3H), 0.93-0.85 (m, 6H), 0.59 (s, 3H). LC-ELSD/MS purity >99%; MS ESI calcd. for C28H₄₈₀₂ [M+H−H₂O]⁺ 400.4, found 400.3.

### Example 59: Synthesis of (3S,5S,8S,10S,13R,14S,17R)-3-ethyl-17-((R)-4-(1-hydroxycyclopentyl) butan-2-yl)-10,13-dimethyl-2,3,4,5,6,7,8,10,12,13,14,15,16,17-tetradecahydro-1H-cyclopenta[a]phenanthren-3-ol (E-6)

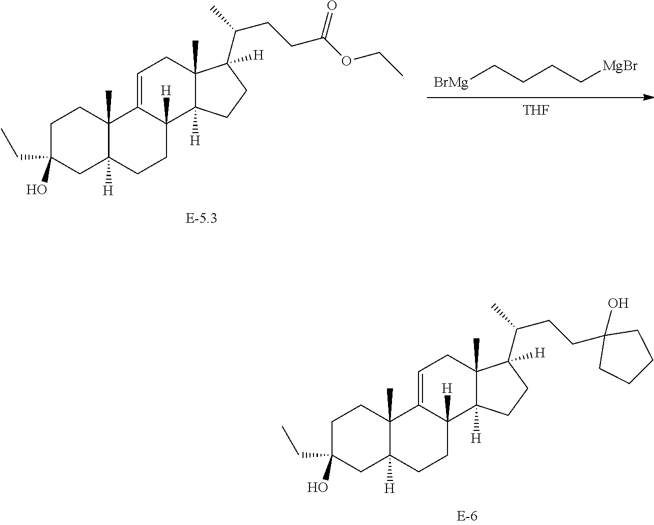

### Synthesis of E-6

A suspension of Mg (960 mg, 40.0 mmol) and I₂ (25.3 mg) in THF (10 ml) was stirred under N₂ at 20° C. To the mixture was added 1,4-dibromobutane (2.15 g, 10 mmol) in THF (10 ml) slowly. After stirring 10 mins, the mixture was heated at 45° C. for 1 hour. The mixture was cooled to 0° C. and E-5.3 (0.100 g, 0.23 mmol) was added and the mixture was stirred for 2 hours. The mixture was quenched with NH₄Cl (10 mL) and extracted with EtOAc (3×10 mL). The combined organic phase was washed with brine (2×20 mL), dried over Na₂SO₄, filtered, and concentrated. The residue was purified by flash column (0˜20% of EtOAc in PE) and triturated with hexane (10 mL) at 20° C. to give E-6 (78.5 mg, 79%). 1H NMR (400 MHz, CDCl₃) δ_{H} 5.24 (d, J=5.6 Hz, 1H), 2.20-2.10 (m, 1H), 2.04-1.59 (m, 15H), 1.55-1.32 (m, 10H), 1.31-1.10 (m, 10H), 0.96 (s, 3H), 0.92 (d, J=6.8 Hz, 3H), 0.90-0.85 (m, 4H), 0.59 (s, 3H). LC-ELSD/MS purity 99%, MS ESI calcd for C₃₀H₅₀₀₂ [M−2H₂O+H]⁺ 407.4, found 407.4.

### Example 60: Synthesis of (3S,5S,8S,10S,13R,14S,17R)-3-ethyl-17-((R)-4-(1-hydroxycyclopropyl) butan-2-yl)-10,13-dimethyl-2,3,4,5,6,7,8,10,12,13,14,15,16,17-tetradecahydro-1H-cyclopenta[a]phenanthren-3-ol (E-7)

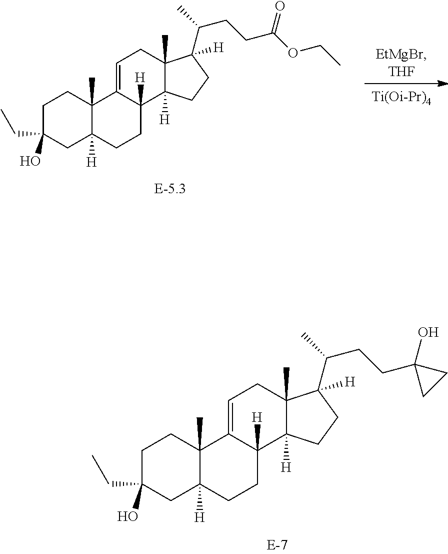

### Synthesis of E-7

To a solution of E-5.3 (150 mg, 0.3482 mmol) in THF (5 mL) was added Ti (i-PrO)+ (394 mg, 1.39 mmol) and EtMgBr (0.58 mL, 3 M in Et₂O, 1.74 mmol) at 25° C. After that, the reaction mixture was stirred at 25° C. for 15 mins under N₂. After stirring at 25° C. for 30 mins the reaction mixture was quenched with saturated NH₄Cl (30 mL) solution and extracted with EtOAc (3×50 mL). The combined organic layer was washed with brine (50 mL), dried over Na₂SO₄, filtered, and concentrated. Purification by silica gel column (0˜20% of EtOAc in PE) provided E-7 (25.9 mg, 17%). ¹H NMR (400 MHz, CDCl₃) δ_{H} 5.24 (d, J=6.0 Hz, 1H), 2.21-2.09 (m, 1H), 1.95-1.83 (m, 3H), 1.79-1.61 (m, 8H), 1.49-1.08 (m, 17H), 0.96 (s, 3H), 0.91 (d, J=6.4 Hz, 3H), 0.90-0.85 (m, 3H), 0.80-0.65 (m, 2H), 0.59 (s, 3H), 0.47-0.35 (m, 2H). LC-ELSD/MS purity >99%, MS ESI calcd. for C28H₄₆₀₂ [M−H₂O+H]⁺ 397.4, found 397.4.

### Example 61: Synthesis of (R)-3-((R)-3-((3S,5S,8S,10S,13R,14S,17R)-3-ethyl-3-hydroxy-10,13-dimethyl-2,3,4,5,6,7,8,10,12,13,14,15,16,17-tetradecahydro-1H-cyclopenta[a]phenanthren-17-yl)butyl)tetrahydrofuran-3-ol (E-8) & (S)-3-((R)-3-((3S,5S,8S,10S,13R,14S,17R)-3-ethyl-3-hydroxy-10,13-dimethyl-2,3,4,5,6,7,8,10,12,13,14,15,16,17-tetradecahydro-1H-cyclopenta[a]phenanthren-17-yl)butyl)tetrahydrofuran-3-ol (E-9)

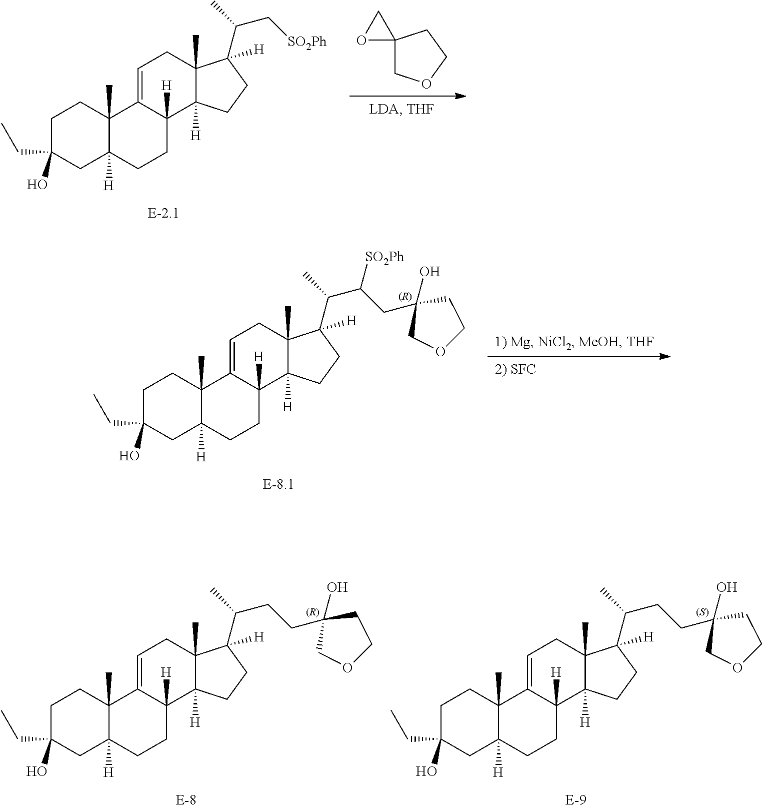

### Synthesis of E-8.1

To a solution of E-2.1 (1.00 g, 2.06 mmol) in anhydrous THF (8 mL) under N₂ at 70° C. was added dropwise n-BuLi (3.29 mL, 8.24 mmol, 2.5 M in hexane). The reaction mixture was stirred at −70° C. for 0.5 hour. Diisopropylamine (0.86 mL, 6.18 mmol) was added dropwise at −70° C. for 0.5 hour followed by addition of 1,5-dioxaspiro[2.4]heptane (267 mg, 2.67 mmol) dropwise at −70° C. The reaction mixture was warmed to 25° C. slowly and stirred at 25° C. for 18 hours. The reaction mixture was quenched with saturated NH₄Cl (50 mL) and extracted with EtOAc (3×10 mL). The combined organic phase was washed with brine (50 mL), dried over Na₂SO₄, filtered, and concentrated to give E-8.1 (0.300 g, 25%).

### Synthesis of E-8 & E-9

To a solution of E-8.1 (0.300 g, 0.5129 mmol) in methanol (10 ml) and THF (5 ml) was added nickel (II) chloride (13.1 mg, 0.102 mmol) and Mg powder (736 mg, 30.7 mmol) in four portions under N₂ at 65° C. The reaction mixture was stirred at 65° C. for 1 hour and then quenched by citric acid (100 mL, 20%) which was added dropwise until solid was dissolved. The mixture was extracted with EtOAc (3×20 mL), the organic layer was washed with brine (100 mL), dried over Na₂SO₄, filtered, and concentrated. The residue was purified by flash column (0˜20% of EtOAc in PE) and separated by SFC (Column: DAICEL CHIRALCEL OD-H (250 mm*30 mm, 5 um); Condition: 0.1% NH₃H₂O ETOH; Begin B: 35%; End B: 35%) to give E-8 (20.0 mg) and E-9 (30.0 mg). E-8 (0.02 g) was purified from MeCN at 25° C. to give E-8 (5.1 mg, 25%). E-9 (30.0 mg, impure) was further purified by flash column chromatography (20˜40% of EA in PE) and purified from MeCN at 25° C. to give E-9 (11.2 mg, 37%).

E-8: ¹H NMR (400 MHz, CDCl₃) δ_{H} 5.25-5.20 (m, 1H), 4.02 (q, J 8.4 Hz, 1H), 3.94-3.84 (m, 1H), 3.70 (d, J 9.6 Hz, 1H), 3.55 (d, J 9.2 Hz, 1H), 2.15 (br dd, J 5.2, 15.6 Hz, 1H), 2.05-1.61 (m, 11H), 1.55-1.48 (m, 4H), 1.45-1.08 (m, 14H), 0.98-0.85 (m, 10H), 0.59 (s, 3H). LC-ELSD/MS purity >99%, MS ESI calcd. for C₂₉H₄₈₀₃ [M−2H₂O+H]⁺ 409.3, found 409.4.

E-9: ¹H NMR (400 MHz, CDCl₃) δ_{H} 5.25-5.20 (m, 1H), 4.02 (q, J 8.0 Hz, 1H), 3.93-3.84 (m, 1H), 3.68 (d, J 9.2 Hz, 1H), 3.54 (d, J 9.6 Hz, 1H), 2.14 (br dd, J 6.0, 16.0 Hz, 1H), 2.02-1.71 (m, 9H), 1.61-1.44 (m, 6H), 1.40-1.08 (m, 14H), 0.99-0.83 (m, 10H), 0.59 (s, 3H). LC-ELSD/MS purity >99%, MS ESI calcd. for C₂₉H₄₈₀₃ [M−H₂O+H]⁺ 427.3, found 427.4.

### Example 62: Synthesis of (3S,5S,8S,10S,13R,14S,17R)-17-((R)-5-hydroxypentan-2-yl)-3,10,13-trimethyl-2,3,4,5,6,7,8,10,12,13,14,15,16,17-tetradecahydro-1H-cyclopenta[a]phenanthren-3-ol (E-10) & (3S,5S,8S,10S,13R,14S,17R)-17-((2R,5R)-5-hydroxy-6-methylheptan-2-yl)-3,10,13-trimethyl-2,3,4,5,6,7,8,10,12,13,14,15,16,17-tetradecahydro-1H-cyclopenta[a]phenanthren-3-ol (E-11) & (3S,5S,8S,10S,13R,14S,17R)-17-((2R,5S)-5-hydroxy-6-methylheptan-2-yl)-3,10,13-trimethyl-2,3,4,5,6,7,8,10,12,13,14,15,16,17-tetradecahydro-1H-cyclopenta[a]phenanthren-3-ol (E-12)

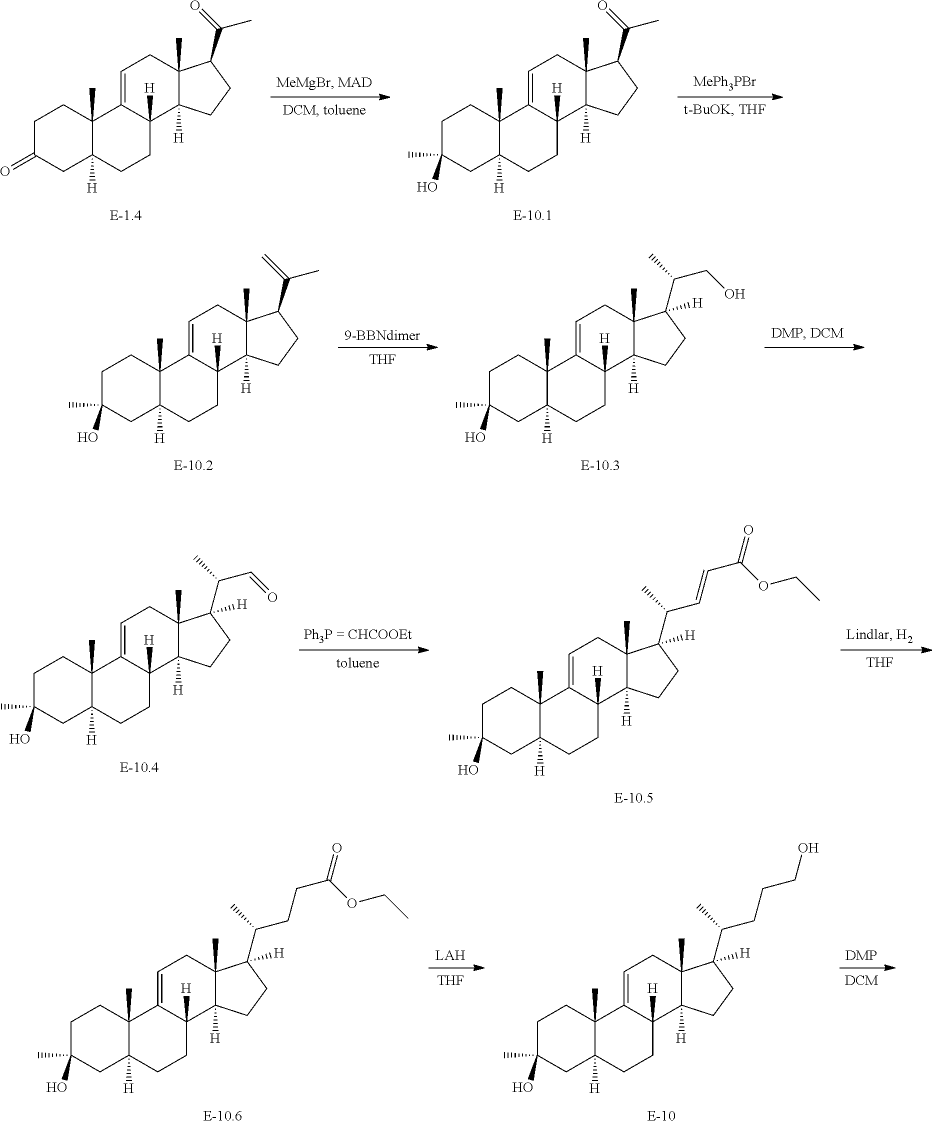

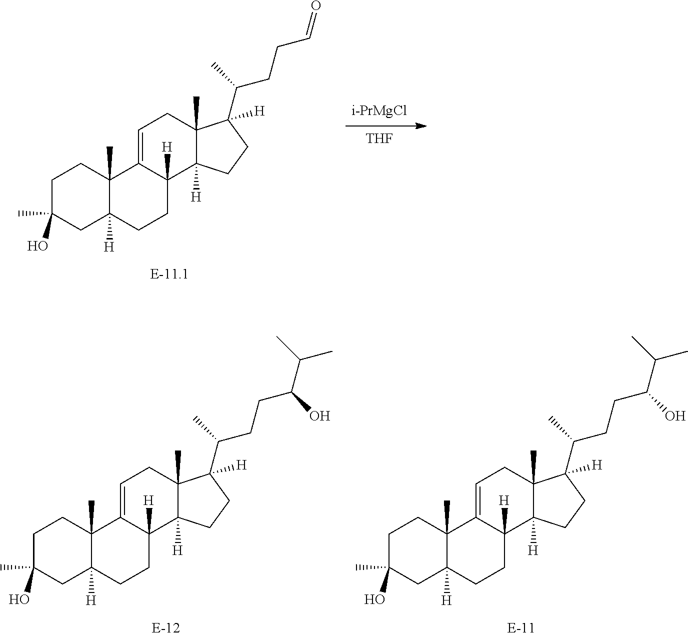

### Synthesis of E-10.1

To a solution of 2,6-di-tert-butyl-4-methylphenol (40.0 g, 181 mmol) in toluene (400 mL) was added dropwise AlMe₃ (45.2 mL, 90.5 mmol, 2 M in toluene) at 0° C. under N₂. The mixture was stirred at 25° C. for 1 hour. To the freshly prepared solution of MAD (48.5 g, 101 mmol) under N₂ at −70° C. was added a solution of E-1.4 (8.00 g, 25.4 mmol) in anhydrous DCM (200 mL) dropwise. The mixture was stirred at −70° C. for 1 hour and then methylmagnesium bromide (3 M in diethyl ether, 33.6 mL, 101 mmol) was added to the above mixture dropwise. After stirring at −70° C. for 2 hours, the reaction mixture was quenched with aqueous citric acid (200 mL), extracted with EtOAc (100 mL) three times, dried over anhydrous sodium sulfate, filtered, and concentrated. Purification by flash column chromatography (0˜45% of EtOAc in PE) provided E-10.1 (8.00 g). ¹H NMR (400 MHz, CDCl₃) δ_{H} 5.25-5.40 (m, 1H), 2.58 (t, J=9.2 Hz, 1H), 2.27-2.23 (m, 3H), 2.13 (s, 3H), 2.06-1.81 (m, 4H), 1.51-1.28 (m, 12H), 1.25-1.21 (m, 4H), 0.94 (s, 3H), 0.55 (s, 3H).

### Synthesis of E-10.2

To a mixture of MePPh₃Br (29.0 g, 81.6 mmol) in THF (500 mL) was added t-BuOK (9.13 g, 81.6 mmol) at 25° C. under N₂. The resulting mixture was stirred at 50° C. for 30 mins then E-10.1 (9.00 g, 27.2 mmol) was added in portions below 50° C. After stirring at 50° C. for 1 hour, the reaction mixture was quenched with 10% NH₄Cl aqueous (300 mL) at 25° C. The aqueous was extracted with EtOAc (400 mL) and the combined organic phase was concentrated and the residue was triturated with MeOH/H₂O (200 mL/200 mL) to give E-10.2 (8.20 g). ¹H NMR (400 MHz, CDCl₃) δ_{H} 5.30 (s, 1H), 4.85 (s, 1H), 4.71 (s, 1H), 2.20-1.80 (m, 6H), 1.71-1.38 (m, 10H), 1.26-1.18 (m, 10H), 0.96-0.93 (m, 4H), 0.50 (s, 3H).

### Synthesis of E-10.3

To a solution of E-10.2 (4.00 g, 12.1 mmol) in THF (50 mL) was added 9-BBN dimer (145 mL, 72.6 mmol, 0.5 M) and the mixture was stirred at 20° C. for 16 hours. To the resulting mixture was added ethanol (8.55 mL, 145 mmol) at 0° C. for 0.5 hour, followed by NaOH aqueous (28.9 mL, 5.0 M, 145 mmol) at 0° C. H₂O₂ (14.4 mL, 10 M, 145 mmol) was added. After stirring at 60° C. for 1 hour, the mixture was added to Na₂SO₃ (100 mL, 15%) and extracted with EtOAc (3×200 mL). The organic layer was washed with brine (200 mL), dried over anhydrous Na₂SO₄, filtered, and concentrated. Purification by flash column (0˜45% of EtOAc in PE) provided E-10.3 (5.50 g). ¹H NMR (400 MHz, CDCl₃) δ_{H} 5.26-5.24 (m, 1H), 3.67-3.62 (m, 1H), 3.44-3.34 (m, 1H), 2.20-2.11 (m, 1H), 2.08-1.92 (m, 3H), 1.90-1.69 (m, 5H), 1.44-1.29 (m, 7H), 1.30-1.20 (m, 9H), 1.04 (d, J 6.8 Hz, 3H), 0.80-1.10 (m, 4H), 0.61 (s, 3H).

### Synthesis of E-10.4

To a solution of E-10.3 (5.50 g, 15.8 mmol) in DCM (200 mL) was added DMP (20.0 g, 47.4 mmol) at 25° C. under N₂. After stirring at 25° C. for 3 hours the reaction mixture was diluted with DCM (200 mL) and carefully quenched with sat. NaHCO₃ (300 mL) and sat. Na₂S₂O₃ (300 mL). After stirring at 25° C. for 5 mins, the organic layer was separated, washed with sat. NaHCO₃ (300 mL) and sat. Na₂S₂O₃ (300 mL), dried over Na₂SO₄, filtered and concentrated to give E-10.4 (5.60 g). ¹H NMR (400 MHz, CDCl₃) δ_{H} 9.55-9.60 (m, 1H), 5.28-5.23 (m, 1H), 2.42-2.32 (m, 1H), 2.12-1.95 (m, 3H), 1.93-1.72 (m, 5H), 1.48-1.31 (m, 7H), 1.28-1.21 (m, 8H), 1.14-1.11 (m, 3H), 0.97-0.91 (m, 4H), 0.64 (s, 3H).

### Synthesis of E-10.5

To a solution of E-10.4 (5.60 g, 16.2 mmol) in toluene (300 mL), ethyl 2-(triphenyl-lambda5-phosphanylidene) acetate (11.2 g, 32.4 mmol) was added. After stirring at 110° C. for 12 hours, the mixture was poured into saturated NH₄Cl (400 mL) and extracted with EtOAc (2×300 mL). The combined organic phase was washed with brine (300 mL), dried over anhydrous Na₂SO₄, filtered, and concentrated. Purification by flash column (0˜30% EtOAc in PE) provided E-10.5 (2.80 g). ¹H NMR (400 MHz, CDCl₃) δ_{H} 6.85 (dd, J=8.8, 15.6 Hz, 1H), 5.76-5.71 (m, 1H), 5.29-5.20 (m, 1H), 4.17 (q, J=7.2 Hz, 2H), 2.32-2.22 (m, 1H), 2.21-2.09 (m, 1H), 2.01-1.84 (m, 3H), 1.76-1.56 (m, 6H), 1.49-1.34 (m, 5H), 1.26 (m, 11H), 1.08 (s, 3H), 0.95-0.92 (m, 4H), 0.63 (s, 3H).

### Synthesis of E-10.6

To a solution of E-10.5 (2.40 g, 5.78 mmol) in THF (20 mL) was added Pd/CaCO3 (2.50 g). After stirring at 25° C. under H₂ for 48 hours, the mixture was filtered, concentrated, and purified by flash column (0˜45% of EtOAc in PE) to give E-10.6 (1.80 g). E-10.6 (70.0 mg) was further purified by SFC (column: DAICEL CHIRALCEL OJ-H (250 mm*30 mm, 10 um), condition: 0.1% NH₃H₂O ETOH, Begin B: 30, End B: 30) to give E-10.6 (24.2 mg, 35%). ¹H NMR (400 MHz, CDCl₃) δ_{H} 5.27-5.25 (m, 1H), 4.12 (q, J=7.2 Hz, 2H), 2.40-2.25 (m, 2H), 2.27-2.08 (m, 3H), 2.00-1.85 (m, 6H), 1.81-1.69 (m, 3H), 1.69-1.59 (m, 3H), 1.42-1.29 (m, 7H), 1.25-1.22 (m, 8H), 0.96-0.93 (m, 6H), 0.59 (s, 3H). LC-ELSD/MS purity >99%, MS ESI calcd for C₂₇H₄₄O₃ [M+H−H₂O]⁺ 399 found 399.

### Synthesis of E-10

To a solution of E-10.6 (1.00 g, 2.66 mmol) in THF (20 mL) was added LiAlH₄ (252 mg, 6.64 mmol) at 25° C. under N₂. After stirring at 25° C. for 2 hours, the mixture was poured into H₂O (30 mL) and extracted with EtOAc (3×30 mL). The combined organic layer was washed with brine (2×20 mL), dried over anhydrous Na₂SO₄, filtered, and concentrated to give E-10 (900 mg). Then E-10 (70.0 mg) was further purified by SFC (column: DAICEL CHIRALCEL OJ-H (250 mm*30 mm, 10 um), condition: 0.1% NH₃H₂O ETOH, Begin B: 35, End B: 35) to give E-10 (23.0 mg). ¹H NMR (400 MHz, CDCl₃) δ_{H} 5.27-5.25 (m, 1H), 3.62-3.60 (m, 2H), 2.25-2.06 (m, 1H), 2.02-1.94 (m, 2H), 1.90-1.80 (m, 2H), 1.75-1.59 (m, 5H), 1.50-1.35 (m, 7H), 1.34-1.26 (m, 4H), 1.25-1.21 (m, 9H), 0.95-0.92 (m, 6H), 0.60 (s, 3H). LC-ELSD/MS purity >99%, MS ESI calcd for C25H₄₂₀₂ [M+H−H₂O]⁺ 357 found 357.

### Synthesis of E-11.1

To a solution of E-10 (500 mg, 1.33 mmol) in DCM (20 mL) was added DMP (1.69 g, 3.99 mmol). After stirring at 40° C. for 2 hours, the reaction mixture was diluted with DCM (50 mL) and carefully quenched with saturated NaHCO₃ (50 mL) and saturated Na₂S₂O₃ (50 mL). After stirring at 20° C. for 5 mins, the organic layer was separated, washed with saturated NaHCO₃ (50 mL) and saturated Na₂S₂O₃ (50 mL), dried over Na₂SO₄, filtered, and concentrated to give E-11.1 (450 mg). ¹H NMR (400 MHz, CDCl₃) δ_{H} 9.80-9.75 (m, 1H), 5.26-5.24 (m, 1H), 2.37-2.21 (m, 1H), 2.21-2.12 (m, 1H), 2.03-1.92 (m, 3H), 1.66-1.54 (m, 5H), 1.50-1.29 (m, 10H), 1.28-1.01 (m, 7H), 0.98-0.90 (m, 7H), 0.59 (s, 3H).

### Synthesis of E-11 & E-12

To a solution of chloro (propan-2-yl) magnesium (1.87 ml, 2 M, 3.75 mmol) in THF (10 mL) under N₂ was added E-11.1 (350 mg, 0.939 mmol) in THF (10 mL) dropwise slowly at 25° C. After stirring at 25° C. for 2 hours the mixture was poured slowly into sat. NH₄Cl solution (20 mL) and stirred for 20 mins. The aqueous phase was extracted with EtOAc (2×30 mL). The combined organic phase was washed with brine (2×20 mL), dried over anhydrous Na₂SO₄, filtered, and concentrated. Purification by flash column (10˜60% of EtOAc in PE) and SFC (column: DAICEL CHIRALCEL OJ-H (250 mm*30 mm, 10 um), condition: 0.1% NH₃H₂O ETOH, Begin B: 25, End B: 25) provided E-12 (20.1 mg) and E-11 (21.3 mg).

E-11: ¹H NMR (400 MHz, CDCl₃) δ_{H} 5.27-5.25 (m, 1H), 3.32-3.31 (m, 1H), 2.22-2.09 (m, 1H), 1.99-1.96 (m, 2H), 1.93-1.83 (m, 2H), 1.76-1.60 (m, 5H), 1.47-1.29 (m, 10H), 1.25-1.23 (m, 10H), 0.97-0.88 (m, 13H), 0.60 (s, 3H). LC-ELSD/MS purity >99%, MS ESI calcd for C28H₄₈₀₂ [M+H−H₂O]⁺ 399 found 399.

E-12: ¹H NMR (400 MHz, CDCl₃) δ_{H} 5.27-5.25 (m, 1H), 3.32-3.31 (m, 1H), 2.17-2.15 (m, 1H), 1.98-1.96 (m, 2H), 1.88-1.86 (m, 2H), 1.78-1.57 (m, 7H), 1.50-1.30 (m, 8H), 1.25-1.23 (m, 10H), 0.96-0.88 (m, 13H), 0.60 (s, 3H). LC-ELSD/MS purity >99%, MS ESI calcd for C28H₄₈₀₂ [M+H−H₂O]⁺ 399 found 399.

### Example 63: Synthesis of (3S,5S,8S,10S,13R,14S,17R)-3,10,13-trimethyl-17-((2R,5S)-6,6,6-trifluoro-5-hydroxy-5-methylhexan-2-yl)-2,3,4,5,6,7,8,10,12,13,14,15,16,17-tetradecahydro-1H-cyclopenta[a]phenanthren-3-ol (E-13) and (3S,5S,8S,10S,13R,14S,17R)-3,10,13-trimethyl-17-((2R,5R)-6,6,6-trifluoro-5-hydroxy-5-methylhexan-2-yl)-2,3,4,5,6,7,8,10,12,13,14,15,16,17-tetradecahydro-1H-cyclopenta[a]phenanthren-3-ol (E-14)

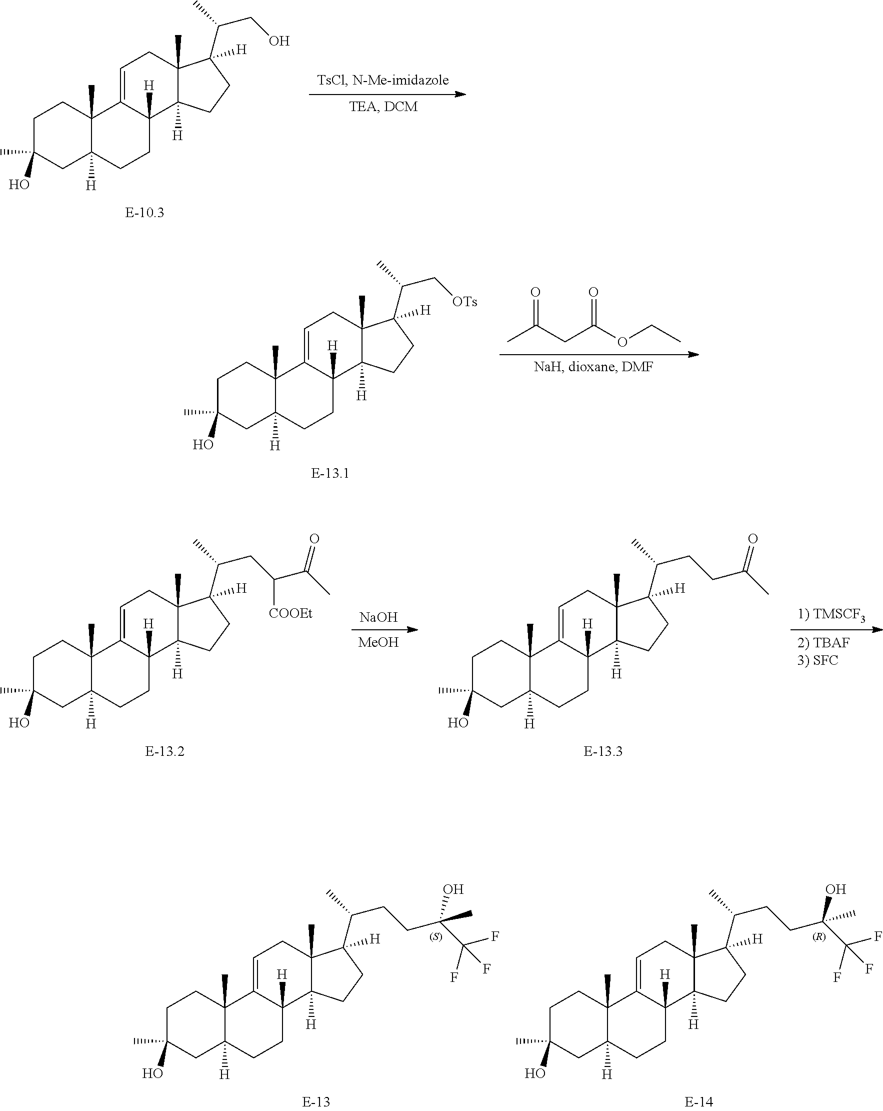

### Synthesis of E-13.1

To a solution of E-10.3 (1 g, 2.88 mmol) in DCM (10 mL) was added N-Me-Imidazole (559 mg, 5.76 mmol), TEA (582 mg, 5.76 mmol) and TsCl (1.64 g, 8.63 mmol) and the resulting mixture was stirred at 20° C. for 2 h. The mixture was washed with water (5 mL), dried over anhydrous Na₂SO₄, filtered, and concentrated to give E-13.1 (1.5 g). ¹HNMR (400 MHz, CDCl₃) δ_{H} 7.78 (d, J=8.0 Hz, 2H), 7.34 (d, J=8.0 Hz, 2H), 5.25-5.19 (m, 1H), 3.98 (dd, J=2.8, 9.2 Hz, 1H), 3.79 (dd, J=6.4, 9.2 Hz, 1H), 2.45 (s, 3H), 2.10-1.83 (m, 4H), 1.73-1.61 (m, 7H), 1.50-1.26 (m, 8H), 1.24 (s, 3H), 1.20-1.06 (m, 3H), 0.97 (d, J=6.4 Hz, 3H), 0.93 (s, 3H), 0.55 (s, 3H).

### Synthesis of E-13.2

To a solution of NaH (331 mg, 60%, 13.8 mmol) in dioxane (10 mL) was added ethyl 3-oxobutanoate (1.97 g, 15.2 mmol) at 0° C. under N₂ and the resulting mixture was stirred for 1 h. DMF (5 mL) and a solution of E-13.1 (700 mg, 1.39 mmol) in dioxane (5 mL) were added dropwise, and the reaction mixture was stirred at 105° C. for 16 h. The mixture was poured into saturated aqueous NH₄Cl (50 mL) and the aqueous phase was extracted with EtOAc (2×50 mL). The combined organic layers were washed with water (2×50 mL), dried over anhydrous Na₂SO₄, filtered, and concentrated to give E-13.2 (700 mg).

### Synthesis of E-13.3

To a mixture of E-13.2 (700 mg, 1.52 mmol) in MeOH (7 mL) was added H₂O (3 mL) and NaOH (600 mg, 15.2 mmol) at 25° C. and the resulting mixture was stirred at 60° C. for 16 h. The reaction mixture was concentrated, and H₂O (20 mL) was added. The mixture was extracted with DCM (3×30 mL) and the combined organic layers were washed with brine (15 mL), dried over anhydrous Na₂SO₄, filtered, and concentrated. The residue was purified by silica gel chromatography (0-20% of EtOAc in PE) to give E-13.3 (270 mg, 31.0%). ¹H NMR (400 MHz, CDCl₃) δ_{H} 5.30-5.16 (m, 1H), 2.51-2.30 (m, 2H), 2.14 (s, 3H), 1.99-1.58 (m, 10H), 1.55-1.25 (m, 11H), 1.24 (s, 3H), 1.21-1.08 (m, 3H), 0.94 (s, 3H), 0.90 (d, J=6.8 Hz, 3H), 0.58 (s, 3H). LC-ELSD/MS 30-90AB_2 min_E, purity>99%, MS ESI calcd. for C₂₋₆H₄₁₀ [M−H₂O+H]⁺ 369.3, found 369.3.

### Synthesis of E-13 and E-14

To a solution of E-13.3 (250 mg, 646 umol) in THF (3 mL) was added TBAF (64.6 μL, IM, 64.6 umol) at 20° C. under N₂. TMSCF₃ (1.37 g, 9.69 mmol) was added dropwise at 20° C. and the resulting mixture was stirred for 2 h. To the mixture was added TBAF (2.58 mL, 1 M, 2.58 mmol, domestic) and the mixture was stirred at 20° C. for another 2 h. Saturated aqueous NH₄Cl (20 mL) was added and the mixture was extracted with EtOAc (2×20 mL). The combined organic layers were washed with water (20 mL), brine (20 mL), dried over anhydrous Na₂SO₄, filtered, and concentrated. The residue was purified by silica gel chromatography (0˜30% EtOAc in PE) and SFC (Column: DAICEL CHIRALPAK AD (250 mm*30 mm, 10 um); Condition: 0.1% NH₃H₂O ETOH; Begin B: 25%; End B: 25%; FlowRate (ml/min): 60; Injections: 60) to give E-13 (57.9 mg, 20.4%) and E-14 (50 mg, 19.4%).

E-13: ¹H NMR (400 MHz, CDCl₃) δ_{H} 5.28-5.22 (m, 1H), 2.21-2.10 (m, 1H), 2.04-1.60 (m, 12H), 1.55-1.34 (m, 9H), 1.32 (s, 3H), 1.24 (s, 3H), 1.22-1.04 (m, 5H), 0.95-0.92 (m, 6H), 0.60 (s, 3H). ¹⁹F NMR (376 MHz, CDCl₃) δ_{F} −83.12. LC-ELSD/MS 30-90AB_2 min_E, purity>99%, MS ESI calcd. for C₂₇H₄₂F₃O [M−H₂O+H]⁺ 439.3, found 439.3.

E-14: ¹H NMR (400 MHz, CDCl₃) δ_{H} 5.31-5.19 (m, 1H), 2.20-2.09 (m, 1H), 2.01-1.59 (m, 11H), 1.54-1.34 (m, 9H), 1.33 (s, 3H), 1.24 (s, 3H), 1.23-1.05 (m, 6H), 0.96-0.91 (m, 6H), 0.60 (s, 3H). ¹⁹F NMR (376 MHz, CDCl₃) δ_{F} −82.80. LC-ELSD/MS 30-90AB_2 min_E, purity>99%, MS ESI calcd. for C₂₇H₄₂F₃O [M−H₂O+H]⁺ 439.3, found 439.3.

### Example 64: Synthesis of (3S,5S,8S,10S,13R,14S,17R)-17-((R)-4-(1-hydroxycyclopropyl) butan-2-yl)-3,10,13-trimethyl-2,3,4,5,6,7,8,10,12,13,14,15,16,17-tetradecahydro-1H-cyclopenta[a]phenanthren-3-ol (E-15)

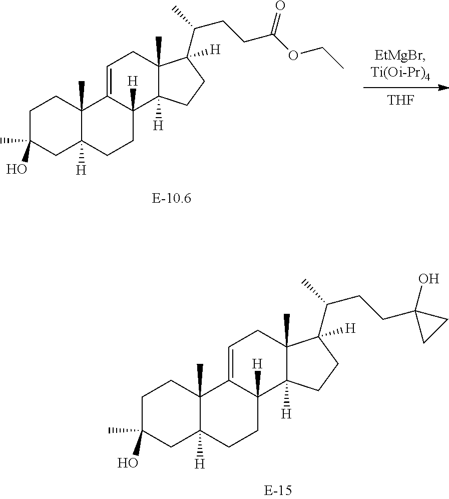

### Synthesis of E-15

To a solution of E-10.6 (300 mg, 0.72 mmol) in THF (10 mL) was added Ti (i-PrO)+ (817 mg, 2.88 mmol) and EtMgBr (1.19 mL, 3.59 mmol, 3 M in Et₂O) at 25° C. and the resulting mixture was stirred for 30 min under N₂. The reaction mixture was quenched with saturated aqueous NH₄Cl (30 mL) and extracted with EtOAc (3×50 mL). The combined organic layers were washed with brine (50 mL), dried over anhydrous Na₂SO₄, filtered, concentrated in vacuo and purified by silica gel chromatography (0˜20% EtOAc in PE) to afford E-15 (28.4 mg, 9.83%). ¹H NMR (400 MHz, CDCl₃) δ_{H} 5.26 (d, J=5.6 Hz, 1H), 2.19-2.12 (m, 1H), 2.02-1.58 (m, 12H), 1.56-1.30 (m, 11H), 1.24 (s, 3H), 1.21-1.10 (m, 3H), 0.94 (s, 3H), 0.92 (d, J=6.4 Hz, 3H), 0.77-0.70 (m, 2H), 0.60 (s, 3H), 0.48-0.39 (m, 2H). LC-ELSD/MS 10-80AB_7 min_E, purity >99%, MS ESI calcd. for C₂₇H₄₃₀ [M−H₂O+H]⁺ 383.4 found 383.4.

### Example 65: Synthesis of (3S,5S,8S,10S,13R,14S,17R)-17-((R)-5-ethyl-5-hydroxyheptan-2-yl)-3,10,13-trimethyl-2,3,4,5,6,7,8,10,12,13,14,15,16,17-tetradecahydro-1H-cyclopenta[a]phenanthren-3-ol (E-16)

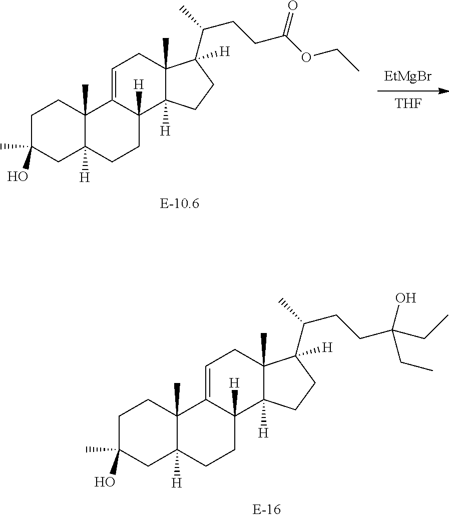

### Synthesis of E-16

A solution of EtMgBr (1.59 mL, 4.80 mmol, 3 M in Et₂O) in THF (4 mL) was reacted with a solution of E-10.6 (200 mg, 0.48 mmol) in THF (2 mL) slowly at 0° C. under N₂ and the resulting mixture was stirred at 20° C. for 1 h. The residue was poured into saturated aqueous NH₄Cl (10 mL) and stirred for 20 min. The layers were separated and the aqueous layer was extracted with DCM (3×20 mL). The combined organic layers were washed with brine (20 mL), dried over anhydrous Na₂SO₄, filtered, concentrated in vacuo and purified by silica gel chromatography (0˜20% EtOAc in PE) to afford E-16 (84.0 mg, 40.8%). ¹HNMR (400 MHz, CDCl₃) δ_{H} 5.26 (d, J=5.6 Hz, 1H), 2.20-2.11 (m, 1H), 2.02-1.85 (m, 4H), 1.74-1.59 (m, 4H), 1.52-1.26 (m, 17H), 1.24 (s, 3H), 1.22-1.01 (m, 5H), 0.95-0.92 (m, 6H), 0.88-0.83 (m, 6H), 0.59 (s, 3H). LC-ELSD/MS 10-80AB_7 min_E, purity >99%, MS ESI calcd. for C₂₉H₄₇ [M−2H₂O+H]⁺ 395.4 found 395.4.

### Example 66

The Formula (F-I) compounds of the disclosure may be synthesized according to Schemes F-1 or F-2.

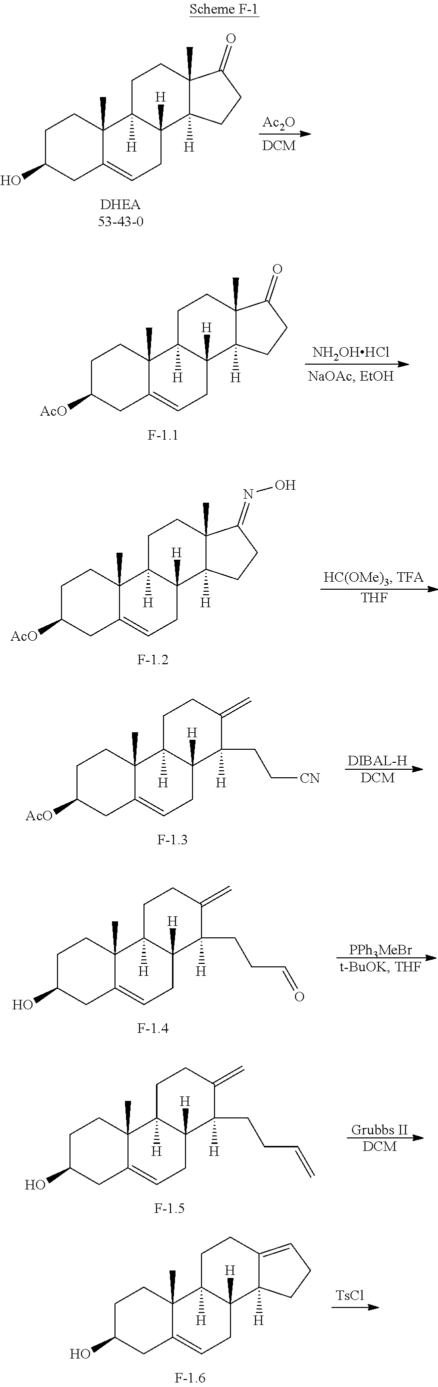

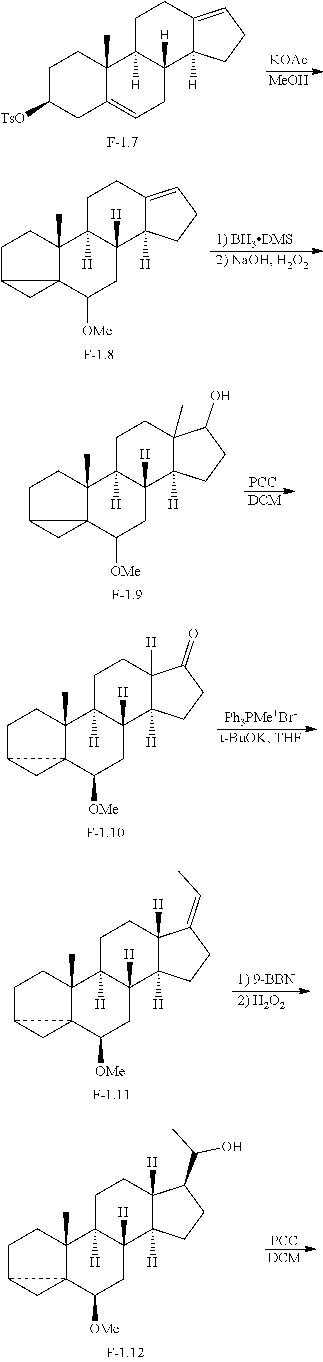

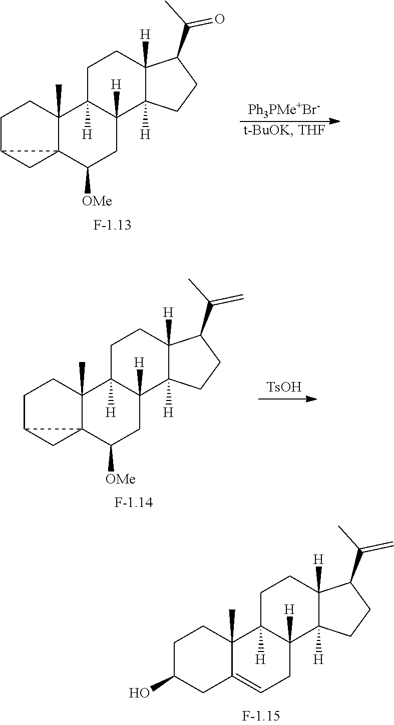

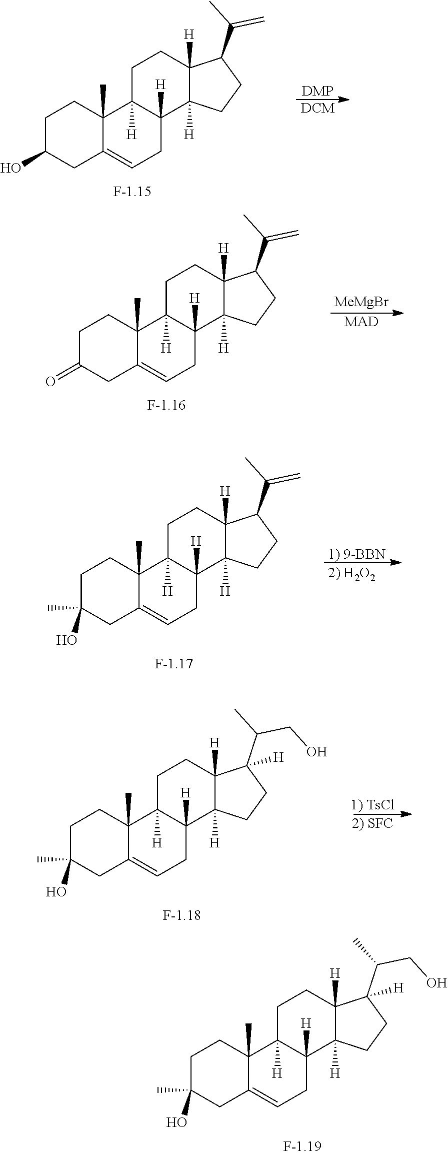

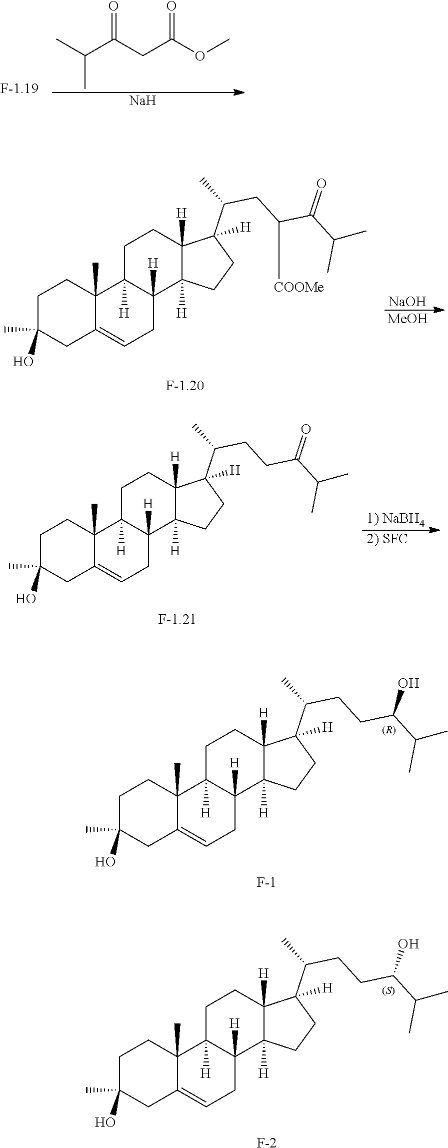

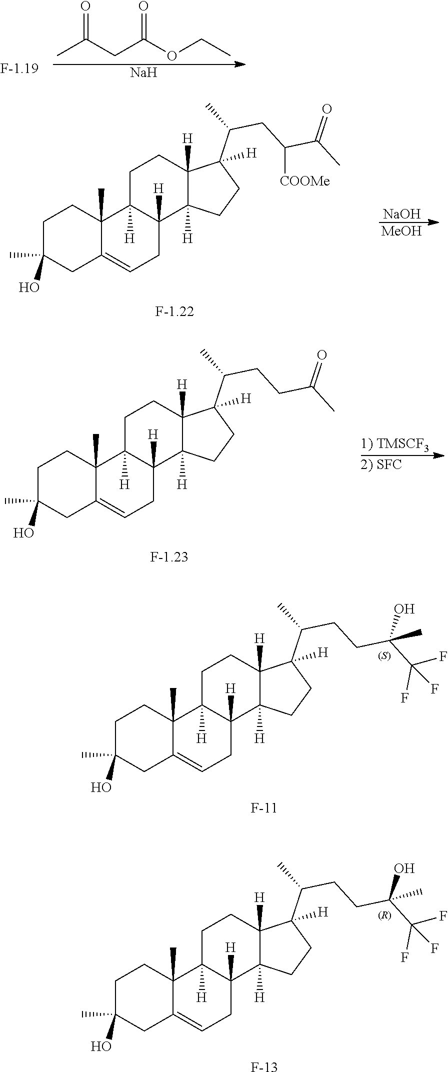

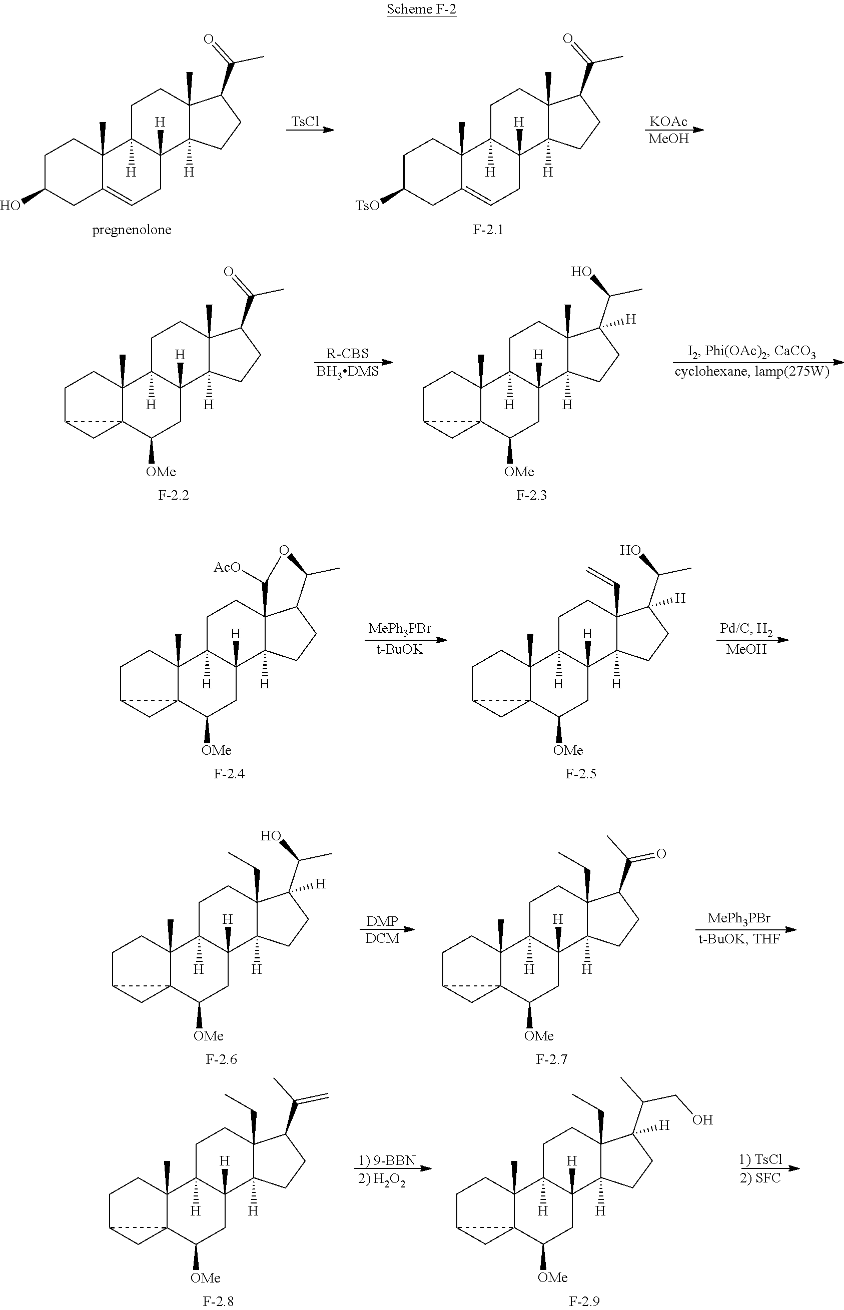

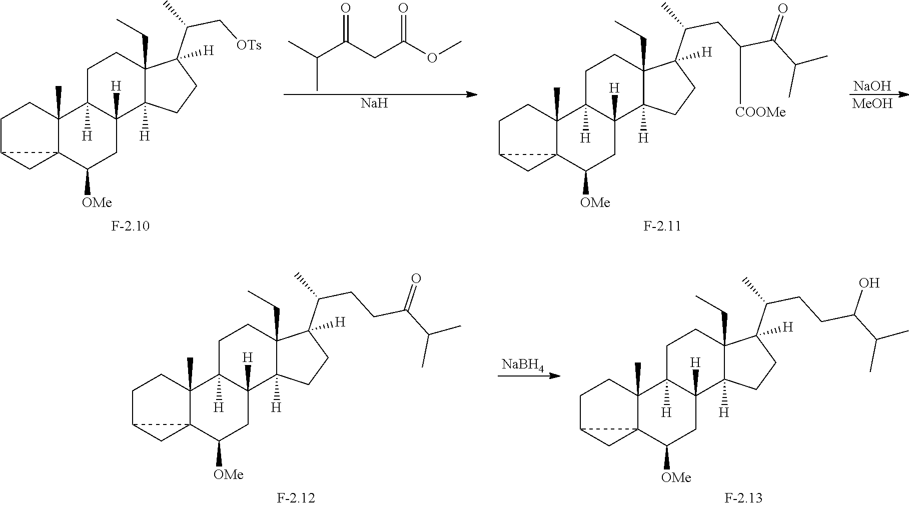

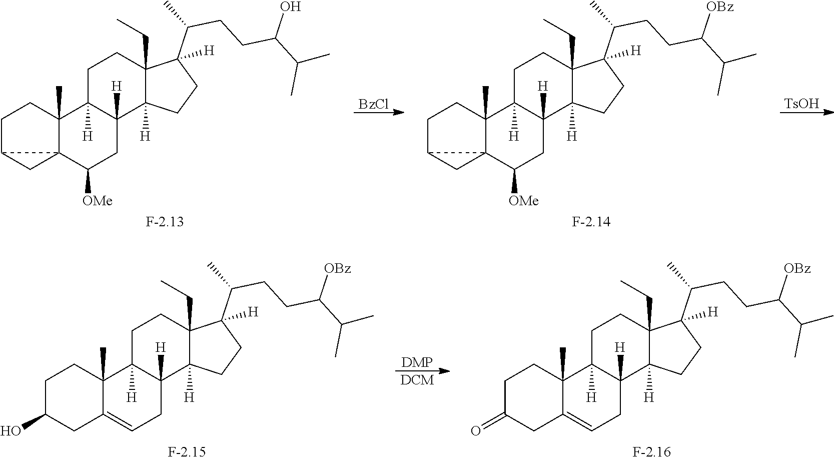

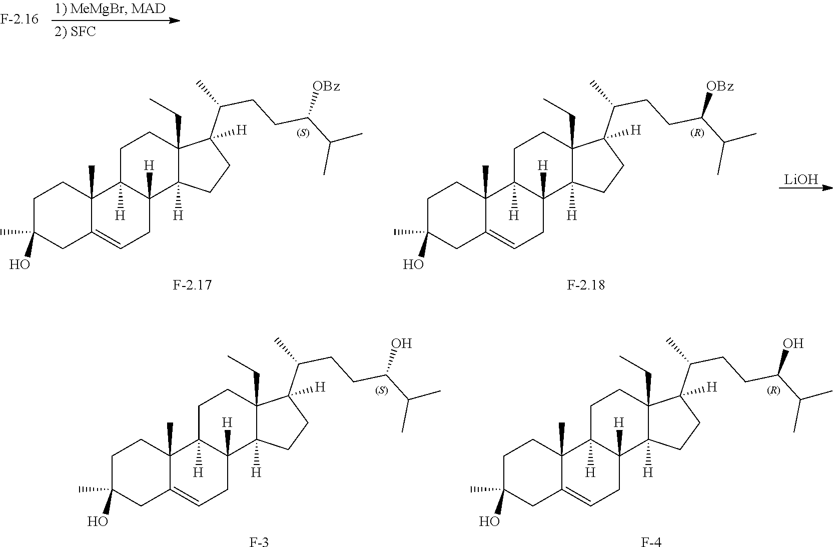

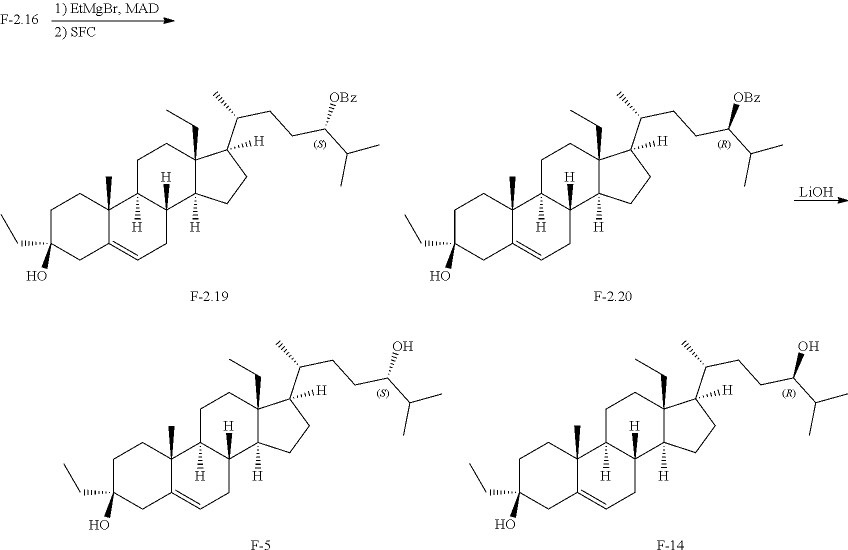

### Example 67: Synthesis of(S)-2-((1aR,3aR,3bS,5aS,6R,8aS,8bS,10R,10aR)-5a-ethyl-10-methoxy-3a-methylhexadecahydrocyclopenta[a]cyclopropa[2,3]cyclopenta[1,2-f]naphthalen-6-yl)propyl 4-methylbenzenesulfonate (F-2.10)

Compounds F-2.1 to F-2.9 of Scheme F-2 may be prepared according to the following protocols:

### Synthesis of F-2.1

To a solution of Pregnenolone (100 g, 315 mmol) in pyridine (800 ml) was added TsCl (180 g, 945 mmol) and DMAP (19.1 g, 157 mmol) at 20° C. and the resulting mixture was stirred for 28 h. Water (200 mL) was added and the mixture was filtered. The filtrate was concentrated to give F-2.1 (140 g, 94.5%). ¹H NMR (400 MHz, CDCl₃) δ_{H} 7.79 (d, J-8.4 Hz, 2H), 7.33 (d, J=8.4 Hz, 2H), 5.32-5.28 (m, 1H), 4.39-4.28 (m, 1H), 2.54-2.47 (m, 1H), 2.44 (s, 3H), 2.42-2.13 (m, 3H), 2.11 (s, 3H), 2.05-1.37 (m, 13H), 1.28-0.99 (m, 3H), 0.96 (s, 3H), 0.61 (s, 3H).

### Synthesis of F-2.2

To a solution of KOAc (91.1 g, 929 mmol) in MeOH (800 mL) was added F-2.1 (80 g, 169 mmol) at 25° C. and the resulting mixture was heated to reflux for 16 h. The mixture was cooled to 25° C. and poured into water (500 mL). The mixture was filtered, and the filtrate was concentrated to give F-2.2 (55 g).

### Synthesis of F-2.3

To a solution of (R)—CBS (3.76 g, 13.6 mmol) in toluene (20 mL) was added a solution of BH₃·SMe₂ (40.8 mL, 10 M, 408 mmol) in toluene (100 mL) and stirred at 0° C. for 30 min under N₂. To the mixture was added a solution of F-2.2 (45 g, 136 mmol) in toluene (100 mL) dropwise at 0° C. and the resulting mixture was stirred for 1 h. MeOH (3 mL) was added dropwise and the mixture was concentrated and combined with another solution of F-2.3 and was purified by silica gel chromatography (0˜2.5% EtOAc in PE) to give F-2.3 (30 g, 66%). ¹H NMR (400 MHz, CDCl₃) δ_{H} 3.77-3.67 (m, 1H), 3.38-3.29 (m, 3H), 2.78 (t, J=2.8 Hz, 1H), 1.97-1.63 (m, 7H), 1.54-1.29 (m, 7H), 1.21-1.04 (m, 1H), 1.21-1.04 (m, 5H), 1.02 (s, 3H), 0.93-0.77 (m, 3H), 0.72 (s, 3H), 0.68-0.63 (m, 1H), 0.47-0.41 (m, 1H).

### Synthesis of F-2.4

To a solution of F-2.3 (7 g, 21 mmol) in cyclohexane (700 mL) were added CaCO3 (6.3 g, 63 mmol), PhI(OAc)₂ (20.2 g, 63 mmol), and I₂ (10.6 g, 42 mmol) at 25° C. under N₂. The mixture was heated to reflux (80° C.) by irradiating with an infrared lamp (250 W) for 30 min. The reaction was cooled to 25° C., filtered, and the cyclohexane solution was quenched with saturated aqueous Na₂S₂O₃ (300 mL). The mixture was extracted with EtOAc (2×300 mL) and the combined organic layers were washed with brine (300 mL), dried over anhydrous Na₂SO₄, filtered, and concentrated to give F-2.4 (8 g) which was used directly in the next step.

### Synthesis of F-2.5

To a mixture of MePPh₃Br (43.9 g, 123 mmol) in THF (500 mL) was added t-BuOK (13.8 g, 123 mmol) at 25° C. under N₂ and the resulting mixture was stirred at 50° C. for 30 min. F-2.4 (8 g, 20.5 mmol) in THF (50 mL) was added in portions to keep the internal temperature below 50° C. After stirring at 50° C. for 2 h, the reaction mixture was poured into water (100 mL) at 25° C. The aqueous phase was extracted with EtOAc (2×100 mL) and the combined organic layers were washed with water (100 mL), brine (200 mL), dried over anhydrous Na₂SO₄, filtered, and concentrated in vacuo. The residue was combined with another batch of F-2.5 and was purified by silica gel chromatography (0-5% EtOAc in PE) to give F-2.5 (12 g). ¹H NMR (400 MHz, CDCl₃) δ_{H} 5.89-5.77 (m, 1H), 5.31 (dd, J=1.4, 11.2 Hz, 1H), 5.17 (dd, J=1.6, 17.8 Hz, 1H), 3.88-3.80 (m, 1H), 3.31 (s, 3H), 2.76 (t, J=3 Hz, 1H), 2.42-2.33 (m, 1H), 1.93-1.74 (m, 8H), 1.55-1.28 (m, 7H), 1.13 (d, J=6.0 Hz, 3H), 1.10-1.06 (m, 1H), 0.94 (s, 3H), 0.92-0.84 (m, 3H), 0.66-0.60 (m, 1H), 0.47-0.37 (m, 1H).

### Synthesis of F-2.6

To a solution of F-2.5 (12 g, 34.8 mmol) in MeOH (100 mL) was added Pd/C (1.2 g, <1% water) and the solution was hydrogenated under 30 psi of hydrogen at 25° C. for 48 h. The mixture was filtered through a pad of celite and the filtrate was concentrated in vacuo to give F-2.6 (12 g). ¹H NMR (400 MHz, CDCl₃) δ_{H} 3.90-3.78 (m, 1H), 3.32 (s, 3H), 2.77 (t, J=2.8 Hz, 1H), 2.19-2.10 (m, 1H), 1.91-1.62 (m, 6H), 1.55-1.33 (m, 5H), 1.27 (d, J-6.0 Hz, 3H), 1.25-1.05 (m, 5H), 1.01 (s, 3H), 0.96-0.78 (m, 8H), 0.66-0.63 (m, 1H), 0.47-0.40 (m, 1H).

### Synthesis of F-2.7

To a solution of F-2.6 (12 g, 34.6 mmol) in DCM (120 mL) was added DMP (29.3 g, 69.2 mmol) and the resulting mixture was stirred at 40° C. for 1 h. The mixture was quenched with saturated aqueous NaHCO₃ (200 mL), and the aqueous layer was extracted with DCM (2×300 mL). The combined organic layers were washed with saturated aqueous Na₂S₂O₃ (2×300 mL), brine (300 mL), dried over anhydrous Na₂SO₄, filtered, and concentrated in vacuo to give F-2.7 (10 g). ¹H NMR (400 MHz, CDCl₃) δ_{H} 3.31 (s, 3H), 2.77 (t, J=2.6 Hz, 1H), 2.46 (t, J=8.8 Hz, 1H), 2.36-2.22 (m, 2H), 2.20 (s, 3H), 1.94-1.63 (m, 4H), 1.60-1.05 (m, 12H), 1.01 (s, 3H), 0.94-0.78 (m, 3H), 0.66-0.61 (m, 3H), 0.48-0.41 (m, 1H).

### Synthesis of F-2.8

To a mixture of MePPh₃Br (62.1 g, 174 mmol) in THF (500 mL) was added t-BuOK (19.5 g, 174 mmol) at 25° C. under N₂ and the resulting mixture was stirred at 50° C. for 30 min. F-2.7 (10 g, 29 mmol) in THF (100 mL) was added in portions to keep the internal temperature below 50° C. The reaction mixture was stirred at 50° C. for 2 h and was poured into water (300 mL) at 25° C. The aqueous phase was extracted with EtOAc (2×300 mL) and the combined organic layers were washed with water (300 mL), brine (200 mL), dried over anhydrous Na₂SO₄, filtered, and concentrated in vacuo. The residue was purified by silica gel chromatography (0-0% EtOAc in PE) to give F-2.8 (8.3 g, 83%). ¹H NMR (400 MHz, CDCl₃) δ_{H} 4.80 (d, J=13.6 Hz, 2H), 3.32 (s, 3H), 2.78 (t, J-2.8 Hz, 1H), 2.28-2.19 (m, 1H), 2.05-1.63 (m, 10H), 1.56-1.06 (m, 10H), 1.01 (s, 3H), 0.93-0.81 (m, 3H), 0.75 (t, J=7.4 Hz, 3H), 0.66-0.63 (m, 1H), 0.46-0.41 (m, 1H).

### Synthesis of F-2.9

To a solution of F-2.8 (200 mg, 0.5838 mmol) in THF (10 mL) was added 9-BBN dimer (280 mg, 1.16 mmol) under N₂ and the resulting mixture was stirred at 50° C. under N₂ for 2 h. The mixture was cooled to 0° C. and ethanol (0.335 mL, 5.83 mmol) and NaOH (1.16 mL, 5 M, 5.83 mmol) were added. H₂O₂ (0.583 mL, 10 M, 5.83 mmol) was added dropwise at 15° C. and the mixture was stirred at 50° C. for 2 h. The mixture was cooled and poured into saturated aqueous Na₂S₂O₃ (50 mL) and stirred for 30 min. The mixture was filtered and concentrated to afford F-2.9 (200 mg), which was used directly in the next step.

### Synthesis of F-2.10

To a solution of F-2.9 (14.0 g, 38.8 mmol) in DCM (140 mL) was added 1-methyl-1H-imidazole (9.51 g, 116 mmol) and TsCl (11.0 g, 58.2 mmol) at 20° C. and the resulting mixture was stirred for 16 h. The mixture was poured into H₂O (100 mL) and extracted with DCM (3×50 mL). The combined organic layers were washed with saturated aqueous NaHCO₃ (100 mL), 0.5 M HCl (100 mL), brine (100 mL), dried over anhydrous Na₂SO₄, filtered, and concentrated to give F-2.10 (19.4 g). ¹H NMR (400 MHz, CDCl₃) δ_{H} 7.79 (d, J=8.4 Hz, 2H), 7.34 (d, J=8.0 Hz, 2H), 4.01-3.93 (m, 1H), 3.83-3.75 (m, 1H), 3.31 (s, 3H), 2.80-2.69 (m, 1H), 2.45 (s, 3H), 2.25-2.13 (m, 1H), 1.90-1.68 (m, 5H), 1.66-1.54 (m, 3H), 1.52-1.31 (m, 5H), 1.24-1.06 (m, 5H), 1.04 (d, J=6.4 Hz, 3H), 0.99 (s, 3H), 0.96-0.89 (m, 2H), 0.84 (t, J=7.6 Hz, 3H), 0.78-0.69 (m, 1H), 0.64 (t, J=4.4 Hz, 1H), 0.43 (dd, J=5.2, 7.6 Hz, 1H).

### Example 68: Synthesis of (6R)-6-((8S,9S,10R,13R,14S,17R)-13-ethyl-10-methyl-3-oxo-2,3,4,7,8,9,10,11,12,13,14,15,16,17-tetradecahydro-1H-cyclopenta[a]phenanthren-17-yl)-2-methylheptan-3-yl benzoate (F-2.16)

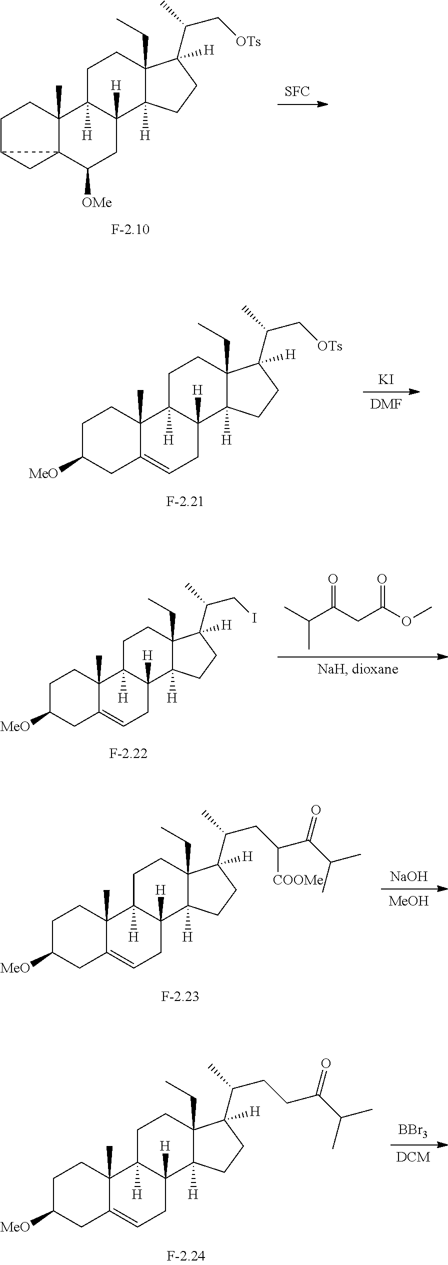

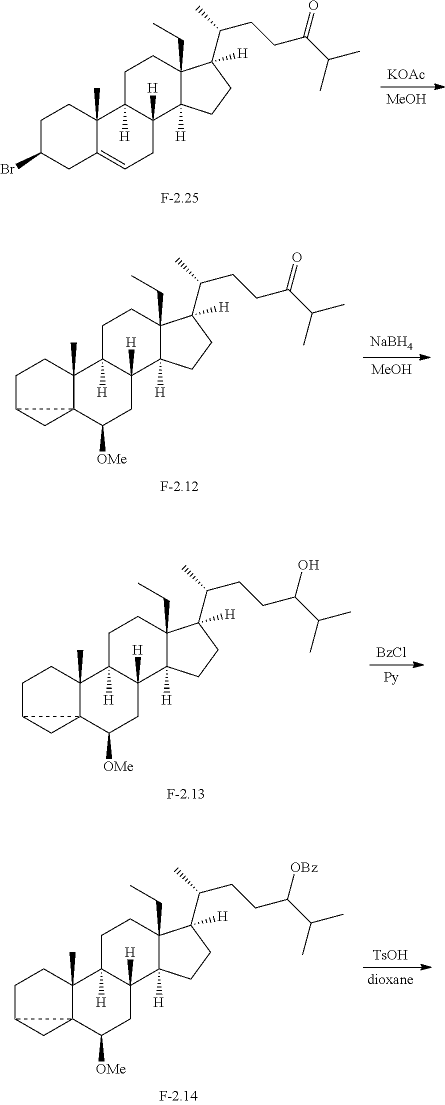

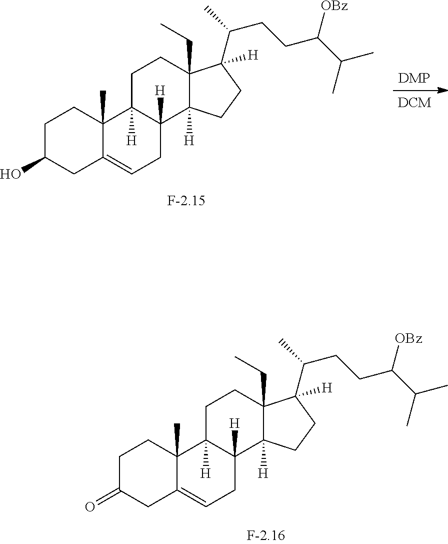

### Synthesis of F-2.21

F-2.10 (19.4 g, 37.6 mmol) was purified by SFC (DAICEL CHIRALPAK IG (250 mm*50 mm, 10 um)). Mobile phase: A: CO₂ B: 0.1% NH₃H₂O ETOH; Gradient: from Gradient: from 40% to 40% of B; flow rate: 200 mL/min to give F-2.21 (10.0 g, 52% yield). ¹H NMR (400 MHz, CDCl₃) δ_{H} 7.78 (d, J=8.4 Hz, 2H), 7.34 (d, J=8.0 Hz, 2H), 5.44-5.25 (m, 1H), 3.97 (dd, J=3.2, 9.2 Hz, 1H), 3.79 (dd, J=6.4, 9.2 Hz, 1H), 3.35 (s, 3H), 3.13-2.99 (m, 1H), 2.45 (s, 3H), 2.41-2.32 (m, 1H), 2.25-2.09 (m, 2H), 1.98-1.74 (m, 4H), 1.73-1.06 (m, 13H), 1.04 (d, J=6.8 Hz, 3H), 1.03-0.99 (m, 2H), 0.96 (s, 3H), 0.95-0.90 (m, 1H), 0.85 (t, J=8.0 Hz, 3H).

### Synthesis of F-2.22

To a solution of F-2.21 (8.8 g, 17.0 mmol) in DMF (130 mL) was added KI (14.1 g, 85.0 mmol) and the resulting mixture was stirred at 60° C. for 1 h. The mixture was cooled and poured into water (300 mL) and stirred for 5 min. The aqueous phase was extracted with EtOAc (2×100 mL) and the combined organic layers were washed with brine (2×100 mL), dried over anhydrous Na₂SO₄, filtered, and concentrated to give F-2.22 (9.0 g). ¹H NMR (400 MHz, CDCl₃) δ_{H} 5.55-5.20 (m, 1H), 3.40-3.27 (m, 4H), 3.25-3.16 (m, 1H), 3.12-3.02 (m, 1H), 2.44-2.34 (m, 1H), 2.27-2.11 (m, 2H), 2.03-1.87 (m, 3H), 1.86-1.82 (m, 2H), 1.66-1.56 (m, 1H), 1.54-1.12 (m, 11H), 1.09 (d, J=6.4 Hz, 3H), 1.08-0.99 (m, 3H), 0.98 (s, 3H), 0.92 (t, J=7.6 Hz, 3H).

### Synthesis of F-2.23

To a solution of NaH (3.81 g, 60%, 95.5 mmol) in dixoane (140 mL) was added methyl 4-methyl-3-oxopentanoate (16.4 g, 114 mmol) at 0° C. under N₂ and the resulting mixture was stirred for 1 h. DMF (70 mL) and a solution of F-2.22 (9.0 g, 19.1 mmol) in DMF (70 mL) were added dropwise. After stirring at 105° C. for 16 h, the mixture was poured into saturated aqueous NH₄Cl (500 mL) and extracted with EtOAc (3×100 mL). The combined organic layers were washed with water (3×100 mL), dried over anhydrous Na₂SO₄, filtered, and concentrated to give F-2.23 (10.0 g), which was used in the next step directly.

### Synthesis of F-2.24

To a solution of F-2.23 (10 g, 20.5 mmol) in MeOH (120 mL) was added H₂O (42.5 mL) and NaOH (8.19 g, 205 mmol) at 25° C. and the resulting mixture was stirred at 60° C. for 16 h. The reaction mixture was concentrated, and H₂O (200 mL) was added. The mixture was extracted with EtOAc (2×100 mL) and the combined organic layers were washed with brine (100 mL), dried over anhydrous Na₂SO₄, filtered, and concentrated. The residue was purified by silica gel chromatography (0-10% of EtOAc in PE) to give F-2.24 (8.7 g).

¹H NMR (400 MHz, CDCl₃) δ_{H} 5.42-5.31 (m, 1H), 3.35 (s, 3H), 3.13-2.99 (m, 1H), 2.67-2.54 (m, 1H), 2.52-2.09 (m, 5H), 2.01-1.57 (m, 6H), 1.55-1.11 (m, 13H), 1.09 (d, J=6.8 Hz, 6H), 1.07-1.01 (m, 3H), 0.99-0.96 (m, 6H), 0.90 (t, J=7.6 Hz, 3H).

### Synthesis of F-2.25

To a solution of F-2.24 (8.7 g, 20.2 mmol) in DCM (90 mL) was added BBr3 (6.06 g, 24.2 mmol) in DCM (45 mL) dropwise at −70° C. and the resulting mixture was stirred at for 2 h. The mixture was added dropwise to 10% aqueous NaHCO₃ (200 mL) and extracted with DCM (2×100 mL). The combined organic layers were washed with H₂O (100 mL×2) and dried over anhydrous Na₂SO₄, filtered, and concentrated to give F-2.25 (9.6 g). ¹H NMR (400 MHz, CDCl₃) δ_{H} 5.45-5.30 (m, 1H), 3.98-3.85 (m, 1H), 2.83-2.69 (m, 1H), 2.66-2.53 (m, 2H), 2.51-2.23 (m, 3H), 2.22-2.13 (m, 1H), 2.04-1.56 (m, 6H), 1.54-1.29 (m, 7H), 1.26-1.12 (m, 5H), 1.09 (d, J=7.2 Hz, 6H), 1.06-1.03 (m, 2H), 1.02 (s, 3H), 0.98 (d, J=6.4 Hz, 3H), 0.96-0.93 (m, 1H), 0.90 (t, J=7.6 Hz, 3H).

### Synthesis of F-2.12

To a suspension of F-2.25 (9.8 g, 20.5 mmol) in MeOH (300 mL) was added KOAc (20.1 g, 205 mmol) and the resulting mixture was then refluxed at 65° C. for 6 days. The mixture was poured into water (500 mL) and extracted with ethyl acetate (150 mL×3). The combined organic layers were washed with brine (200 mL), dried over anhydrous Na₂SO₄, filtered, and concentrated in vacuo. The product was purified by silica gel chromatography (0-3% of EtOAc in PE) to give F-2.12 (7.0 g, 95% yield over 5 steps). ¹H NMR (400 MHz, CDCl₃) δ_{H} 3.31 (s, 3H), 2.81-2.74 (m, 1H), 2.67-2.55 (m, 1H), 2.48-2.24 (m, 3H), 1.95-1.66 (m, 6H), 1.54-1.11 (m, 12H), 1.09 (d, J=6.8 Hz, 6H), 1.06-1.02 (m, 1H), 1.00 (s, 3H), 0.97 (d, J=6.4 Hz, 3H), 0.92-0.87 (m, 5H), 0.86-0.75 (m, 2H), 0.64 (t, J=4.8 Hz, 1H), 0.43 (dd, J=5.2, 7.6 Hz, 1H).

### Synthesis of F-2.13

To a solution of F-2.12 (7.0 g, 16.3 mmol) in MeOH (140 mL) was added NaBH₄ (1.53 g, 40.7 mmol) at 0° C. under N₂ and the resulting mixture was stirred at 25° C. for 2 h. The reaction mixture was quenched with 10% aqueous NH₄Cl (100 mL) and the aqueous layer was extracted with EtOAc (2×80 mL). The combined organic layers were washed with brine (50 mL), dried over anhydrous Na₂SO₄, filtered, and concentrated to give F-2.13 (7.0 g). ¹H NMR (400 MHz, CDCl₃) δ_{H} 3.34-3.25 (m, 4H), 2.80-2.73 (m, 1H), 2.34-2.21 (m, 1H), 1.93-1.85 (m, 1H), 1.81-1.57 (m, 5H), 1.53-1.23 (m, 10H), 1.21-1.08 (m, 5H), 1.01-0.97 (m, 7H), 0.94-0.85 (m, 13H), 0.82-0.76 (m, 1H), 0.64 (t, J=4.8 Hz, 1H), 0.43 (dd, J=5.2, 8.0 Hz, 1H).

### Synthesis of F-2.14

To a solution of F-2.13 (7.0 g, 16.2 mmol) in pyridine (70 mL) was added BzCl (3.71 mL, 32.4 mmol, 1.22 g/mL) and the resulting mixture was stirred at 25° C. for 16 h. The reaction mixture was poured into H₂O (140 mL) and the mixture was extracted with EtOAc (2×100 mL). The combined organic layers were washed with 10% aqueous HCl (100 mL), water (100 mL), brine (100 mL), dried over anhydrous Na₂SO₄, filtered, and concentrated to give F-2.14 (8.8 g), which was used directly in the next step.

### Synthesis of F-2.15

To a solution of F-2.14 (8.0 g, 14.9 mmol) in dioxane (80 mL) was added a solution of TsOH (566 mg, 2.98 mmol) in water (24 mL) and the resulting mixture was stirred at 75° C. for 16 h. NaHCO₃ (100 mL, sat. aq.) was added and the mixture was extracted with EtOAc (150 mL). The organic layer was separated, dried over anhydrous Na₂SO₄, filtered, concentrated, and purified by silica gel chromatography (SiO₂, Petroleum ether/Ethyl acetate=5/1) to give F-2.15 (5.0 g, 59% yield over 3 steps). ¹H NMR (400 MHz, CDCl₃) δ_{H} 8.12-7.97 (m, 2H), 7.59-7.52 (m, 1H), 7.48-7.40 (m, 2H), 5.37-5.32 (m, 1H), 4.99-4.92 (m, 1H), 3.57-3.46 (m, 1H), 2.33-2.17 (m, 3H), 2.01-1.58 (m, 6H), 1.55-1.35 (m, 10H), 1.34-1.05 (m, 8H), 1.00-0.96 (m, 13H), 0.93-0.85 (m, 4H).

### Synthesis of F-2.16

To a solution of F-2.15 (0.9 g, 1.72 mmol) in DCM (15 mL) was added DMP (1.45 g, 3.44 mmol) and the resulting mixture was stirred at 25° C. for 1 h. The reaction was combined with another batch and quenched with saturated aqueous NaHCO₃ (50 mL) at 10° C. and DCM (50 mL) was added. The DCM phase was separated and washed with saturated aqueous NaHCO₃/Na₂S₂O₃ (1:1, 2×50 mL), brine (50 mL), dried over anhydrous Na₂SO₄, filtered, and concentrated to give F-2.16 (0.9 g, 45% yield). ¹H NMR (400 MHz, CDCl₃) δ_{H} 8.11-7.99 (m, 2H), 7.61-7.51 (m, 1H), 7.48-7.40 (m, 2H), 5.37-5.30 (m, 1H), 5.04-4.88 (m, 1H), 3.33-3.20 (m, 1H), 2.81 (dd, J=2.0, 16.4 Hz, 1H), 2.54-2.41 (m, 1H), 2.35-2.24 (m, 2H), 2.09-1.90 (m, 3H), 1.79-1.65 (m, 2H), 1.56-1.18 (m, 11H), 1.16-1.14 (m, 3H), 1.13-1.02 (m, 4H), 1.02-0.86 (m, 15H).

### Example 69: Synthesis of (3S,8S,9S,10R,13R,14S,17R)-13-ethyl-17-((2R,5S)-5-hydroxy-6-methylheptan-2-yl)-3,10-dimethyl-2,3,4,7,8,9,10,11,12,13,14,15,16,17-tetradecahydro-1H-cyclopenta[a]phenanthren-3-ol (F-3) and (3S,8S,9S,10R,13R,14S,17R)-13-ethyl-17-((2R,5R)-5-hydroxy-6-methylheptan-2-yl)-3,10-dimethyl-2,3,4,7,8,9,10,11,12,13,14,15,16,17-tetradecahydro-1H-cyclopenta[a]phenanthren-3-ol (F-4)

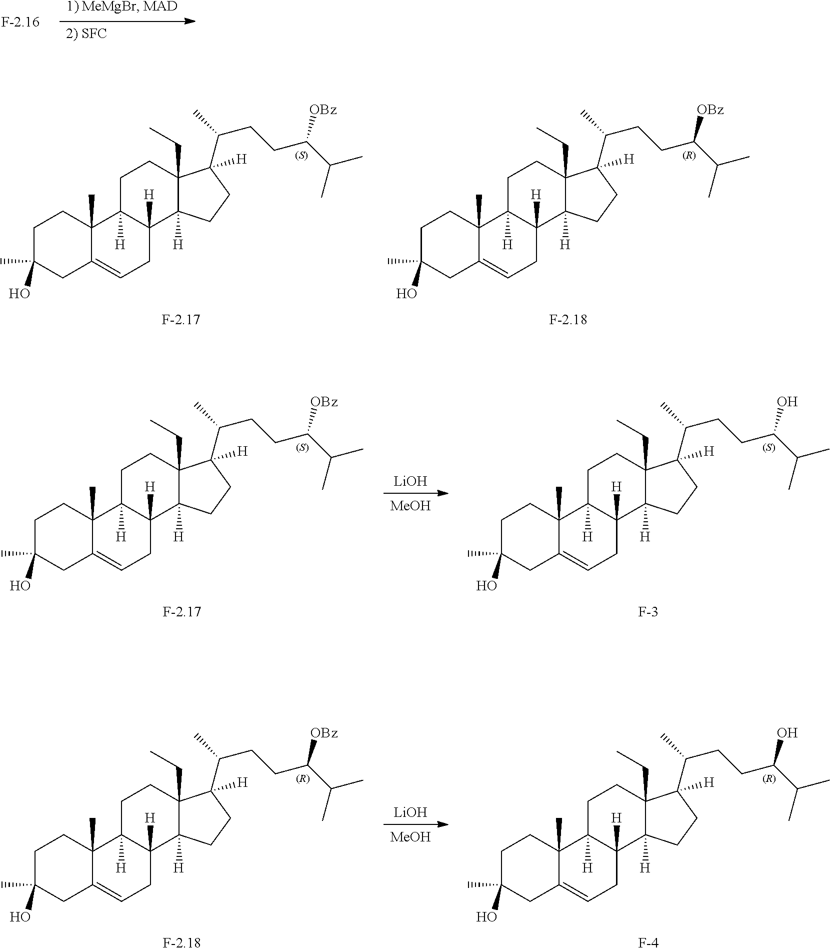

### Synthesis of F-2.17 and F-2.18

To a solution of BHT (1.52 g, 6.92 mmol) in toluene (4 mL) under nitrogen at 0° C. was added AlMe₃ (2 M in toluene, 1.73 mL, 3.46 mmol) dropwise and the resulting mixture was stirred at 20° C. for 1 h. To the freshly prepared MAD (3.46 mmol, in 4 mL toluene) solution was added F-2.16 (450 mg, 0.867 mmol) in DCM (4 mL) dropwise at −70° C. After stirring at −70° C. for 1 h under N₂, MeMgBr (1.44 mL, 4.33 mmol, 3M in ethyl ether) was added dropwise at −70° C. and the resulting solution was stirred at −70° C. for 1 h. The reaction mixture was poured into citric acid (50 mL, 20%) at 10° C. and extracted with EtOAc (2×60 mL). The combined organic layers were washed with brine (2×60 mL) and dried over anhydrous Na₂SO₄, filtered, and concentrated. The residue was purified by silica gel chromatography (0˜15% EtOAc in PE) and further purified by SFC (DAICEL CHIRALPAK AD (250 mm*30 mm, 10 um)). Mobile phase: A: CO₂ B: 0.1% NH₃H₂O IPA; Gradient: from 30% to 30% of B; flow rate: 80 mL/min to give F-2.17 (120 mg, 26% yield) and F-2.18 (120 mg, 26% yield).

F-2.17: ¹H NMR (400 MHz, CDCl₃) δ_{H} 8.08-8.02 (m, 2H), 7.58-7.52 (m, 1H), 7.49-7.41 (m, 2H), 5.36-5.17 (m, 1H), 5.03-4.85 (m, 1H), 2.47-2.36 (m, 1H), 2.31-2.21 (m, 1H), 2.00-1.89 (m, 3H), 1.81-1.65 (m, 4H), 1.62-1.28 (m, 12H), 1.23-1.13 (m, 2H), 1.11 (s, 3H), 1.09-1.02 (m, 2H), 1.02-0.91 (m, 16H), 0.87 (t, J=7.6 Hz, 3H).

F-2.18: ¹H NMR (400 MHz, CDCl₃) δ_{H} 8.09-8.01 (m, 2H), 7.60-7.53 (m, 1H), 7.47-7.41 (m, 2H), 5.36-5.24 (m, 1H), 5.03-4.91 (m, 1H), 2.48-2.36 (m, 1H), 2.30-2.21 (m, 1H), 2.01-1.87 (m, 3H), 1.80-1.66 (m, 4H), 1.61-1.28 (m, 12H), 1.24-1.12 (m, 3H), 1.11 (s, 3H), 1.08-0.93 (m, 17H), 0.90 (t, J=7.2 Hz, 3H).

### Synthesis of F-3

A solution of F-2.17 (120 mg, 0.224 mmol) in THF (5 mL) and MeOH (2.5 mL) was added LiOH (320 mg, 13.4 mmol) and H₂O (2.5 mL) and the resulting mixture was stirred at 50° C. for 16 h. The reaction mixture was poured into H₂O (30 mL) and extracted with EtOAc (30 mL×2). The combined organic layers were washed with brine (30 mL), dried over anhydrous Na₂SO₄, filtered, and concentrated. The residue was purified by silica gel chromatography (3˜15% EtOAc in PE) to give F-3 (73.6 mg, 76% yield). ¹H NMR (400 MHz, CDCl₃) δ_{H} 5.36-5.24 (m, 1H), 3.38-3.24 (m, 1H), 2.46-2.36 (m, 1H), 2.33-2.24 (m, 1H), 2.02-1.92 (m, 2H), 1.84-1.57 (m, 7H), 1.55-1.13 (m, 14H), 1.11 (s, 3H), 1.10-0.98 (m, 10H), 0.97-0.88 (m, 10H). LC-ELSD/MS 30-90AB_2 min_E, purity>99%, MS ESI calcd. for C₂₉H₄₉₀ [M−H₂O+H]⁺ 413.4, found 413.4.

### Synthesis of F-4

To a solution of F-2.18 (120 mg, 0.224 mmol) in THF (5 mL) and MeOH (2.5 mL) was added LiOH (320 mg, 13.4 mmol) and H₂O (2.5 mL) and the resulting mixture was stirred at 50° C. for 16 h. The reaction mixture was poured into H₂O (30 mL) and extracted with EtOAc (30 mL×2). The combined organic layers were washed with brine (30 mL), dried over anhydrous Na₂SO₄, filtered, and concentrated. The residue was purified by silica gel chromatography (3˜15% EtOAc in PE) to give F-4 (89.7 mg, 93% yield). ¹H NMR (400 MHz, CDCl₃) δ_{H} 5.36-5.25 (m, 1H), 3.37-3.25 (m, 1H), 2.46-2.37 (m, 1H), 2.33-2.24 (m, 1H), 2.03-1.58 (m, 7H), 1.54-1.33 (m, 10H), 1.32-1.14 (m, 6H), 1.11 (s, 3H), 1.10-0.98 (m, 9H), 0.98-0.87 (m, 11H). LC-ELSD/MS 30-90AB_2 min_E, purity>99%, MS ESI calcd. for C₂₉H₄₉₀ [M−H₂O+H]⁺ 413.4, found 413.4.

### Example 70: (3S,8S,9S,10R,13R,14S,17R)-3,13-diethyl-17-((2R,5S)-5-hydroxy-6-methylheptan-2-yl)-10-methyl-2,3,4,7,8,9,10,11,12,13,14,15,16,17-tetradecahydro-1H-cyclopenta[a]phenanthren-3-ol (F-5) and (3S,8S,9S,10R,13R,14S,17R)-3,13-diethyl-17-((2R,5R)-5-hydroxy-6-methylheptan-2-yl)-10-methyl-2,3,4,7,8,9,10,11,12,13,14,15,16,17-tetradecahydro-1H-cyclopenta[a]phenanthren-3-ol (F-14)

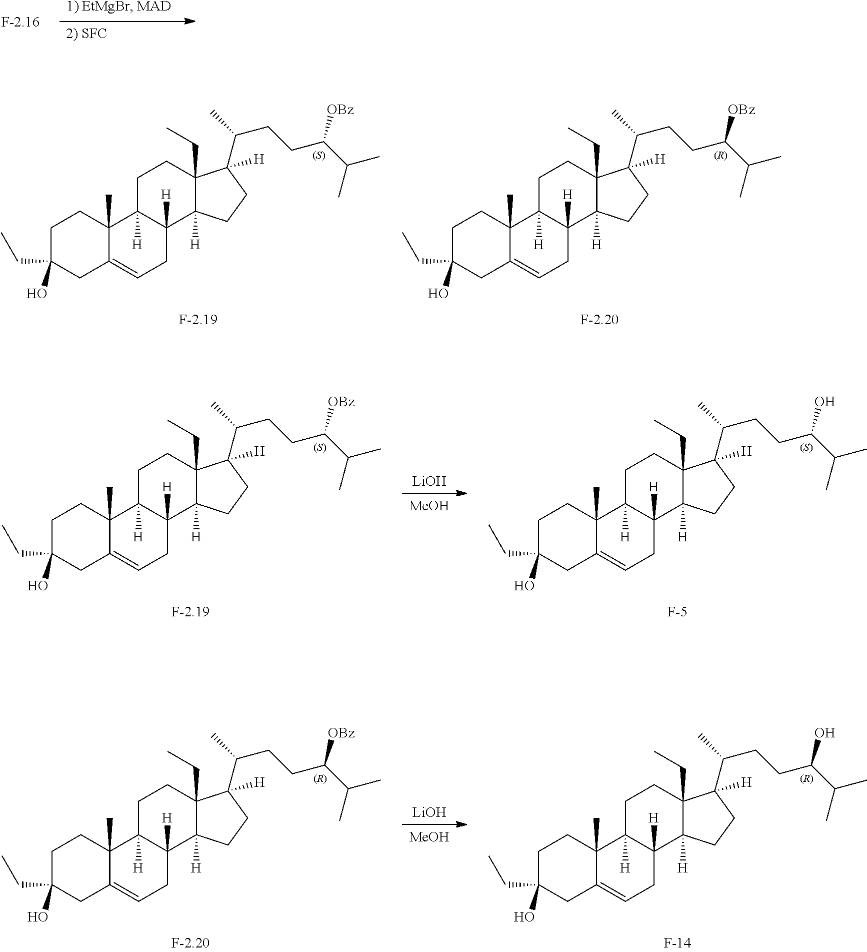

### Synthesis of F-2.19 and F-2.20

To a solution of BHT (1.52 g, 6.92 mmol) in toluene (4 mL) under nitrogen at 0° C. was added AlMe₃ (2 M in toluene, 1.73 mL, 3.46 mmol) dropwise and the resulting mixture was stirred at 20° C. for 1 h. To the freshly prepared MAD (3.46 mmol, in 4 mL toluene) solution was added F-2.16 (450 mg, 0.867 mmol) in DCM (4 mL) dropwise at −70° C. After stirring at −70° C. for 1 h under N₂, EtMgBr (1.44 mL, 4.33 mmol, 3M in ethyl ether) was added dropwise at -70° C. and the resulting solution was stirred at −70° C. for 1 h. The reaction mixture was poured into citric acid (50 mL, 20%) at 10° C. and extracted with EtOAc (2×60 mL). The combined organic layers were washed with brine (2×60 mL) and dried over anhydrous Na₂SO₄, filtered, and concentrated. The residue was purified by silica gel chromatography (0-15% of EtOAc in PE) and further purified by SFC (DAICEL CHIRALCEL OD-H (250 mm*30 mm, 5 um)). Mobile phase: A: CO₂ B: 0.1% NH₃H₂O IPA; Gradient: from 30% to 30% of B; flow rate: 80 mL/min to F-2.19 (120 mg, 25% yield) and F-2.20 (120 mg, 25% yield).

F-2.19: ¹H NMR (400 MHz, CDCl₃) δ_{H} 8.09-8.00 (m, 2H), 7.60-7.51 (m, 1H), 7.48-7.41 (m, 2H), 5.30-5.25 (m, 1H), 5.01-4.91 (m, 1H), 2.40-2.31 (m, 1H), 2.29-2.21 (m, 1H), 2.01-1.90 (m, 2H), 1.77-1.28 (m, 17H), 1.24-1.03 (m, 6H), 1.02-0.91 (m, 16H), 0.89-0.81 (m, 6H).

F-2.20: ¹H NMR (400 MHz, CDCl₃) δ_{H} 8.13-7.99 (m, 2H), 7.59-7.52 (m, 1H), 7.48-7.41 (m, 2H), 5.33-5.25 (m, 1H), 5.01-4.92 (m, 1H), 2.39-2.22 (m, 2H), 2.04-1.90 (m, 3H), 1.80-1.44 (m, 14H), 1.40-1.08 (m, 9H), 1.01-0.94 (m, 15H), 0.90 (t, J=7.6 Hz, 3H), 0.84 (t, J=7.2 Hz, 3H).

### Synthesis of F-5

A solution of F-2.19 (120 mg, 0.218 mmol) in THF (5 mL) and MeOH (2.5 mL) was added LiOH (311 mg, 13.0 mmol) and H₂O (2.5 mL) and the resulting mixture was stirred at 50° C. for 16 h. The reaction mixture was poured into H₂O (30 mL) and extracted with EtOAc (30 mL×2). The combined organic layers were washed with brine (30 mL), dried over anhydrous Na₂SO₄, filtered, and concentrated. The residue was purified by silica gel chromatography (3˜15% of EtOAc in PE) to give F-5 (76.1 mg, 78.5% yield). ¹H NMR (400 MHz, CDCl₃) δ_{H} 5.34-5.20 (m, 1H), 3.39-3.25 (m, 1H), 2.40-2.33 (m, 1H), 2.32-2.23 (m, 1H), 2.06-2.00 (m, 1H), 1.99-1.91 (m, 1H), 1.83-1.45 (m, 14H), 1.42-1.03 (m, 11H), 1.02-0.96 (m, 8H), 0.96-0.88 (m, 10H), 0.85 (t, J=7.6 Hz, 3H). LC-ELSD/MS 30-90AB_2 min_E, purity>99%, MS ESI calcd. for C₃₀H₅₁O [M−H₂O+H]⁺ 427.4, found 427.4.

### Synthesis of F-14

A solution of F-2.20 (120 mg, 0.218 mmol) in THF (5 mL) and MeOH (2.5 mL) was added LiOH (311 mg, 13.0 mmol) and H₂O (2.5 mL) and the resulting mixture was stirred at 50° C. for 16 h. The reaction mixture was poured into H₂O (30 mL) and extracted with EtOAc (30 mL×2). The combined organic layers were washed with brine (30 mL), dried over anhydrous Na₂SO₄, filtered, and concentrated. The residue was purified by silica gel chromatography (3˜15% of EtOAc in PE) to give F-14 (84.0 mg, 86.6% yield).

¹H NMR (400 MHz, CDCl₃) δ_{H} 5.33-5.25 (m, 1H), 3.36-3.26 (m, 1H), 2.40-2.32 (m, 1H), 2.32-2.24 (m, 1H), 2.03 (dd, J=2.0, 13.2 Hz, 1H), 1.99-1.57 (m, 6H), 1.55-1.28 (m, 13H), 1.26-1.02 (m, 8H), 1.02-0.97 (m, 7H), 0.96-0.89 (m, 10H), 0.85 (t, J=7.2 Hz, 3H).

LC-ELSD/MS 30-90AB_2 min_E, purity>99%, MS ESI calcd. for C₃₀H₅₁O [M−H₂O+H]⁺ 427.4, found 427.4.

### Example 71: (3S,5S,8R,9S,10S,13R,14S,17R)-3,13-diethyl-17-((2R,5S)-5-hydroxy-6-methylheptan-2-yl)-10-methylhexadecahydro-1H-cyclopenta[a]phenanthren-3-ol (F-6) and (3S,5S,8R,9S,10S,13R,14S,17R)-3,13-diethyl-17-((2R,5R)-5-hydroxy-6-methylheptan-2-yl)-10-methylhexadecahydro-1H-cyclopenta[a]phenanthren-3-ol (F-7)

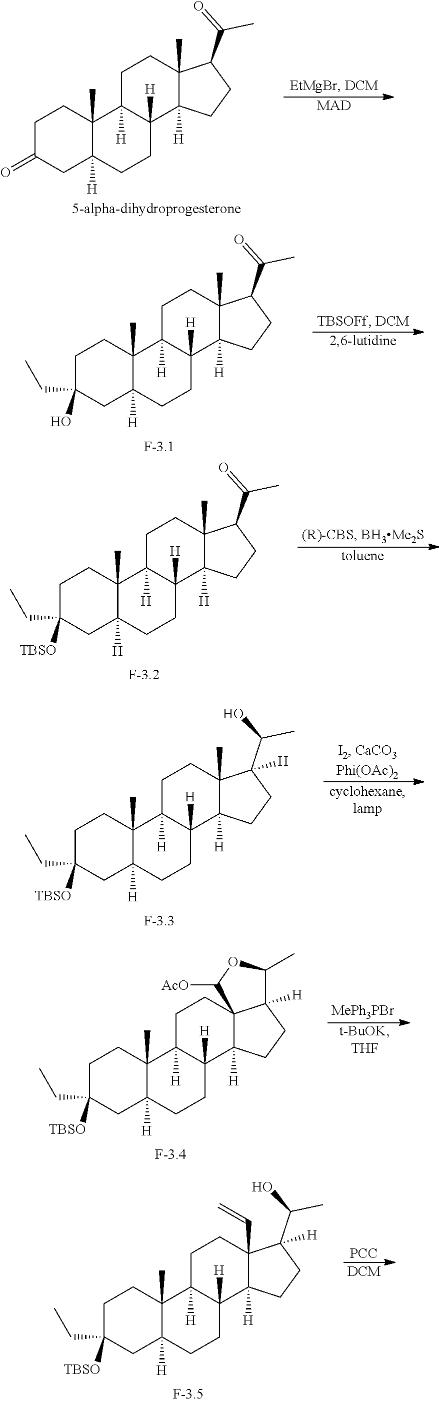

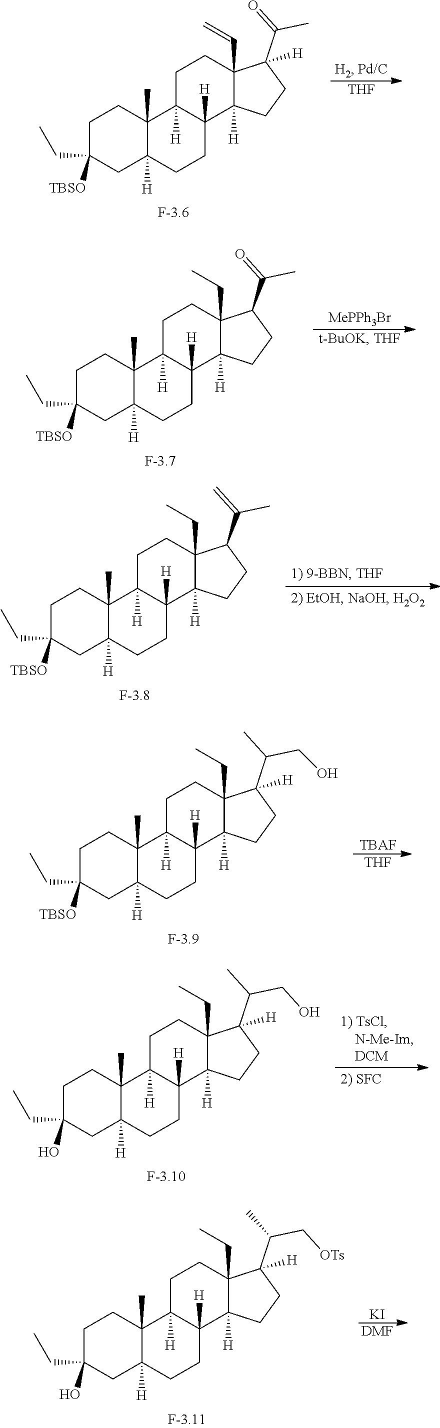

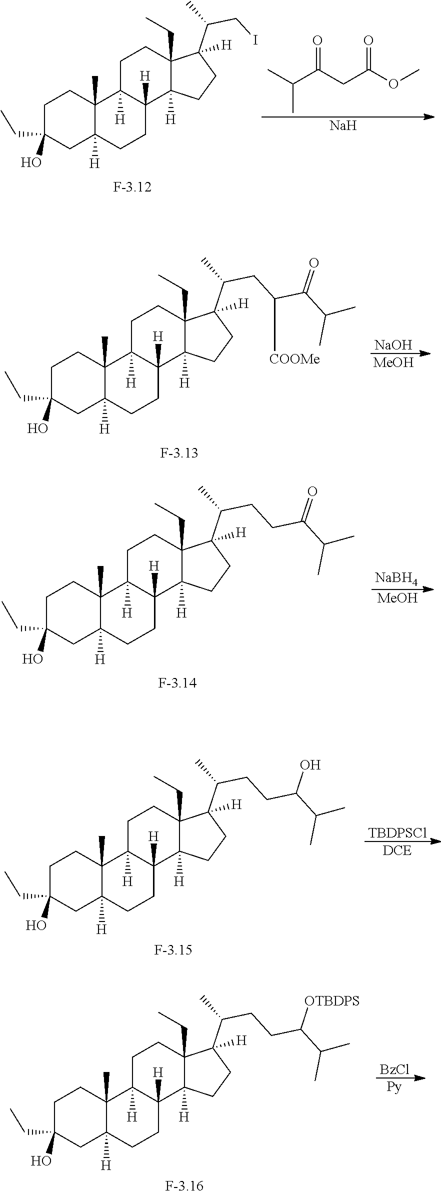

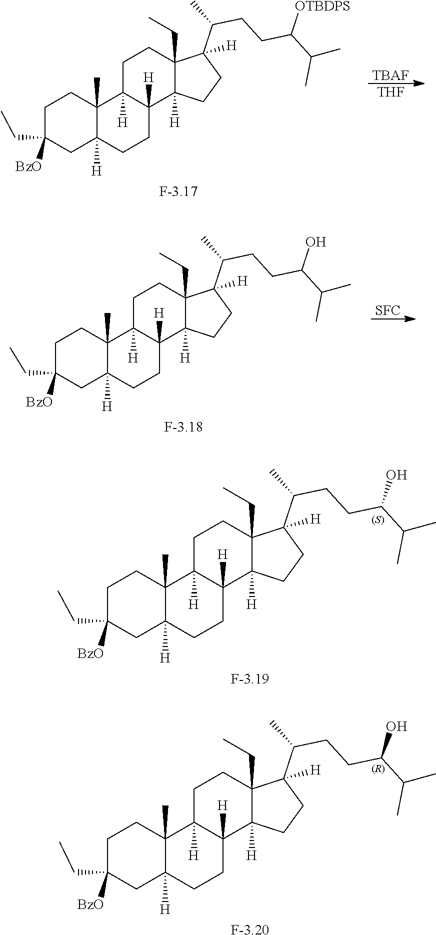

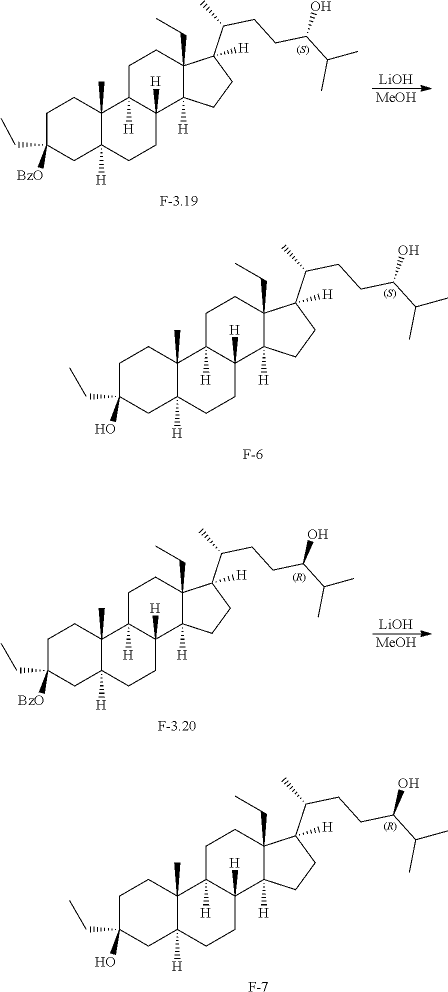

### Synthesis of F-3.1

To a solution of BHT (207 g, 940 mmol) in toluene (400 mL) under nitrogen at 0° C. was added AlMes (2 M in toluene, 235 mL, 470 mmol) dropwise and the resulting mixture was stirred at 25° C. for 1 h. To the MAD (470 mmol in 635 mL toluene) solution was added a solution of 5-α-dihydroprogesterone (60 g, 189 mmol) in DCM (200 mL) dropwise at −70° C. and the mixture was stirred for 1 h under N₂. EtMgBr (156 mL, 470 mmol, 3M in ethyl ether) was added dropwise at −70° C. and the resulting solution was stirred for 4 h. The reaction mixture was poured into saturated aqueous citric acid (1000 mL) at 10° C. and extracted with EtOAc (2×600 mL). The combined organic layers were dried over anhydrous Na₂SO₄, filtered, concentrated, and purified by silica gel chromatography (0˜25% EtOAc in PE) to give F-3.1 (42.7 g).

### Synthesis of F-3.2

To a solution of F-3.1 (40 g, 115 mol) in DCM (1000 ml) was added 2,6-lutidine (24.6 g, 230 mmol) and TBSOTf (45.4 g, 172 mmol) and the resulting mixture was stirred at 40° C. for 16 h. The mixture was poured into water (500 mL), then extracted with DCM (3×300 mL). The combined organic layers were washed with brine (300 mL), dried over anhydrous Na₂SO₄, filtered, concentrated, and purified by silica gel chromatography (0˜10% EtOAc in PE) to give F-3.2 (48 g, 90.7%). ¹H NMR (400 MHz, CDCl₃) δ_{H} 2.56-2.47 (m, 1H), 2.21-2.13 (m, 1H), 2.11 (s, 3H), 2.03-1.94 (m, 1H), 1.72-1.61 (m, 3H), 1.60-1.45 (m, 7H), 1.45-1.27 (m, 5H), 1.26-1.01 (m, 6H), 1.00-0.89 (m, 2H), 0.87-0.82 (m, 10H), 0.81-0.80 (m, 3H), 0.72-0.64 (m, 1H), 0.60 (s, 3H), 0.07 (s, 6H).

### Synthesis of F-3.3

To a solution of (R)—CBS (9.61 g, 34.7 mmol) in toluene (100 mL) was added a solution of BH₃·Me₂S (20.8 mL, 10 M, 208 mmol) and the resulting mixture was stirred at 15° C. for 30 min under N₂. To the mixture was added a suspension of F-3.2 (32 g, 69.4 mmol) in toluene (200 mL) dropwise at 0° C. and the mixture was stirred at 0° C. for 2 h and 15° C. for 16 h. To the mixture was added MeOH (300 mL) dropwise. The mixture was concentrated in vacuo and purified by silica gel chromatography (0˜8% EtOAc in PE/DCM (3:1)) to give F-3.3 (27 g, 84.1%). ¹H NMR (400 MHz, CDCl₃) δ_{H} 3.76-3.64 (m, 1H), 1.95-1.79 (m, 2H), 1.72-1.60 (m, 2H), 1.60-1.55 (m, 4H), 1.53-1.42 (m, 5H), 1.42-1.30 (m, 5H), 1.29-1.24 (m, 1H), 1.21 (d, J=6.4 Hz, 3H), 1.19-1.03 (m, 5H), 1.03-0.98 (m, 1H), 0.97-0.92 (m, 1H), 0.92-0.88 (m, 1H), 0.92-0.88 (m, 1H), 0.85 (s, 9H), 0.80 (s, 3H), 0.65 (s, 3H), 0.64-0.58 (m, 1H), 0.07 (s, 6H).

### Synthesis of F-3.4

To a solution of F-3.3 (3 g, 6.48 mmol) in cyclohexane (300 mL) was added CaCO3 (1.94 g, 19.4 mmol), PhI(OAc)₂ (6.24 g, 19.4 mmol), and I₂ (3.27 g, 12.9 mmol) at 25° C. under N₂. The mixture was heated to reflux (80° C.) by irradiating with an infrared lamp (250 W) for 30 min. The mixture was quenched with saturated aqueous Na₂S₂O₃ (200 mL) and the aqueous layer was extracted with EtOAc (2×100 mL). The combined organic layers were washed with brine (100 mL), dried over anhydrous Na₂SO₄, filtered, and concentrated to give F-3.4 (4 g), which was used directly in next step without purification.

### Synthesis of F-3.5

To a solution of MePh3PBr (8.25 g, 23.1 mmol) in THF (20 mL) was added t-BuOK (2.59 g, 23.1 mmol) at 25° C. and the resulting mixture was stirred at 50° C. for 1 h. A solution of F-3.4 (4 g, 7.7 mmol) in THF (10 mL) was added to the reaction mixture at 50° C. and the mixture was stirred for 16 h. The mixture was poured into saturated aqueous NH₄Cl (50 mL), and the aqueous layer was extracted with EtOAc (3×50 mL). The combined organic layers were washed with brine (100 mL), dried over anhydrous Na₂SO₄, filtered, concentrated, and purified by silica gel chromatography (0˜15% of EtOAc in PE) to give F-3.5 (4 g). ¹H NMR (400 MHz, CDCl₃) δ_{H} 5.77 (dd, J=11.2, 18.0 Hz, 1H), 5.32-5.27 (m, 1H), 5.15 (dd, J=1.6, 18.0 Hz, 1H), 3.82 (t, J=7.6 Hz, 1H), 2.39-2.29 (m, 1H), 1.85-1.64 (m, 5H), 1.54-1.47 (m, 4H), 1.47-1.35 (m, 5H), 1.34-1.27 (m, 2H), 1.24-1.14 (m, 3H), 1.12 (d, J=6.4 Hz, 3H), 1.09-1.00 (m, 2H), 0.98-0.87 (m, 3H), 0.85 (s, 9H), 0.84-0.79 (m, 3H), 0.72 (s, 3H), 0.70-0.65 (m, 1H), 0.06 (s, 6H).

### Synthesis of F-3.6

To a solution of F-3.5 (8.8 g, 18.5 mmol) in DCM (100 mL) was added silica gel (20 g) and PCC (11.9 g, 55.4 mmol) at 25° C. and the resulting mixture was stirred for 1 h. The suspension was filtered, and the filter cake was washed with DCM (2×100 mL). The combined filtrate was concentrated to give F-3.6 (7.9 g, 90.3%). ¹H NMR (400 MHz, CDCl₃) δ_{H} 5.56 (dd, J=11.2, 17.6 Hz, 1H), 5.18 (d, J=11.6 Hz, 1H), 4.97 (dd, J=1.2, 16.8 Hz, 1H), 2.60 (t, J-9.2 Hz, 1H), 2.54-2.48 (m, 1H), 2.25-2.14 (m, 1H), 2.05 (s, 3H), 1.79-1.63 (m, 3H), 1.55-1.50 (m, 4H), 1.47-1.38 (m, 4H), 1.37-1.29 (m, 4H), 1.28-1.24 (m, 1H), 1.22-1.15 (m, 2H), 1.14-0.92 (m, 3H), 0.89-0.87 (m, 1H), 0.85 (s, 9H), 0.83-0.80 (m, 2H), 0.80-0.70 (m, 4H), 0.07 (s, 6H).

### Synthesis of F-3.7

To a solution of F-3.6 (7.3 g, 15.4 mmol) in THF (100 mL) was added Pd/C (dry, 1 g) under N₂. The suspension was degassed under vacuum and purged with H₂ three times. The mixture was stirred under H₂ (50 psi) at 50° C. for 72 h. The reaction mixture was filtered through a pad of Celite and washed with THF (2×100 mL). The filtrate was concentrated to give F-3.7 (7.3 g). ¹H NMR (400 MHz, CDCl₃) δ_{H} 2.43 (t, J=8.8 Hz, 1H), 2.33-2.26 (m, 1H), 2.19 (s, 4H), 1.70-1.58 (m, 4H), 1.55-1.49 (m, 3H), 1.44-1.40 (m, 1H), 1.39-1.31 (m, 2H), 1.31-1.14 (m, 8H), 1.14-1.05 (m, 2H), 1.04-0.88 (m, 3H), 0.86 (s, 9H), 0.85-0.81 (m, 3H), 0.79 (s, 3H), 0.72-0.67 (m, 1H), 0.63 (t, J=7.6 Hz, 3H), 0.07 (s, 6H).

### Synthesis of F-3.8

To a solution of MePh3PBr (12.8 g, 36 mmol) in THF (30 mL) was added t-BuOK (4.03 g, 36 mmol) at 25° C. and the resulting mixture was stirred at 50° C. for 1 h. A solution of F-3.7 (5.7 g, 12 mmol) in THF (20 mL) was added to the reaction mixture at 50° C. and the mixture was stirred for 16 h. The mixture was poured into saturated aqueous NH₄Cl (50 mL), and the aqueous layer was extracted with EtOAc (3×50 mL). The combined organic layers were washed with brine (100 mL), dried over anhydrous Na₂SO₄, filtered, concentrated, and purified by silica gel chromatography (0˜15% of EtOAc in PE) to give to give F-3.8 (5.5 g). ¹H NMR (400 MHz, CDCl₃) δ_{H} 4.80 (s, 1H), 4.77 (s, 1H), 2.24-2.17 (m, 1H), 1.99-1.83 (m, 2H), 1.81 (s, 3H), 1.68-1.57 (m, 4H), 1.55-1.48 (m, 4H), 1.47-1.36 (m, 2H), 1.36-1.30 (m, 1H), 1.27-1.24 (m, 1H), 1.24-1.17 (m, 3H), 1.17-1.03 (m, 5H), 1.01-0.89 (m, 3H), 0.86 (s, 9H), 0.85-0.81 (m, 3H), 0.79 (s, 3H), 0.74 (t, J=7.6 Hz, 3H), 0.69-0.61 (m, 1H), 0.07 (s, 6H).

### Synthesis of F-3.9

To a solution of F-3.8 (5.5 g, 11.6 mmol) in THF (55 mL) was added 9-BBN dimer (8.49 g, 34.8 mmol) at 25° C. and the resulting mixture was stirred for 1 h. To the mixture was added ethanol (5.29 g, 115 mmol) at 0° C. Then aqueous NaOH (23 mL, 5M, 115 mmol) was added at 0° C. followed by H₂O₂ (11.5 mL, 10M, 115 mmol) dropwise. After the additions, the mixture was stirred at 80° C. for 1 h. To the mixture was added saturated aqueous Na₂S₂O₃ (30 mL) and the mixture was stirred for 30 min. The mixture was extracted with EtOAc (30 mL), and the combined organic layers were washed with brine (2×20 mL), dried over anhydrous Na₂SO₄, filtered, and concentrated. The residue was purified by silica gel chromatography (0-60% EtOAc in PE) to give F-3.9 (4.8 g, 84.3%).

### Synthesis of F-3.10

To a suspension of F-3.9 (6.8 g, 13.8 mmol) in THF (30 mL) was added TBAF (27.6 mL, 17.6 mmol) and the resulting mixture was stirred at 80° C. for 16 h. The mixture was quenched with saturated aqueous NH₄Cl (10 mL) and extracted with EtOAc (2×30 mL). The combined organic layers were washed with water (3×30 mL), dried over anhydrous Na₂SO₄, filtered, concentrated, and purified by silica gel chromatography (0˜15% of EtOAc in PE) to give F-3.10 (2.9 g). ¹H NMR (400 MHz, CDCl₃) δ_{H} 3.67-3.58 (m, 1H), 3.42-3.26 (m, 1H), 2.28-2.10 (m, 1H), 1.80-1.59 (m, 5H), 1.56-1.46 (m, 4H), 1.45-1.14 (m, 13H), 1.10 (d, J=6.4 Hz, 3H), 1.08-0.96 (m, 4H), 0.95-0.85 (m, 8H), 0.81 (s, 3H), 0.68-0.57 (m, 1H).

### Synthesis of F-3.11

To a solution of F-3.10 (4.9 g, 5.04 mmol) in DCM (50 mL) was added N-Me-Im (13.17 g, 135.9 mmol) and TsCl (11.49 g, 60.3 mmol) and the resulting mixture was stirred at 20° C. for 2 h. The mixture was washed with water (10 mL), and the organic phase was dried over anhydrous Na₂SO₄, filtered, concentrated. The residue was purified by silica gel chromatography (0˜20% of EtOAc in PE) and separated by SFC (Column: Phenomenex-Cellulose-2 (250 mm*50 mm, 10 um); Condition: 0.1% NH₃H₂O MEOH; Begin B: 45%; End B: 45%) to give F-3.11 (3 g, 66.8%). ¹H NMR (400 MHz, CDCl₃) δ_{H} 7.78 (d, J=8.4 Hz, 2H), 7.78 (d, J=8.0 Hz, 2H), 3.96 (dd, J=3.2, 9.2 Hz, 1H), 3.78 (dd, J=6.4, 9.2 Hz, 1H), 2.45 (s, 3H), 2.20-2.12 (m, 1H), 1.81-1.70 (m, 1H), 1.66-1.58 (m, 4H), 1.56-1.43 (m, 5H), 1.42-1.17 (m, 10H), 1.13-1.05 (m, 2H), 1.03 (d, J=6.4 Hz, 3H), 1.00-0.90 (m, 4H), 0.87 (t, J=7.2 Hz, 3H), 0.85-0.81 (m, 3H), 0.79 (s, 3H), 0.65-0.56 (m, 1H).

### Synthesis of F-3.12

To a solution of F-3.11 (3 g, 5.65 mmol) in DMF (20 mL) was added KI (4.49 g, 27.1 mmol) at 25° C. and the resulting mixture was stirred at 60° C. for 5 h. The mixture was poured into water (50 mL) and stirred for 20 min. The aqueous phase was extracted with EtOAc (3×50 mL), and the combined organic layers were washed with brine (2×50 mL), dried over anhydrous Na₂SO₄, filtered, and concentrated to give F-3.12 (2.3 g). ¹H NMR (400 MHz, CDCl₃) δ_{H} 3.32 (dd, J=2.4, 9.6 Hz, 1H), 3.19 (dd, J=5.2, 9.6 Hz, 1H), 2.22-2.15 (m, 1H), 1.89-1.75 (m, 1H), 1.68-1.58 (m, 4H), 1.57-1.46 (m, 4H), 1.44-1.30 (m, 5H), 1.26-1.10 (m, 8H), 1.08 (d, J=6.0 Hz, 3H), 1.05-0.92 (m, 4H), 0.92-0.86 (m, 7H), 0.81 (s, 3H), 0.69-0.59 (m, 1H).

### Synthesis of F-3.13

To a suspension of NaH (406 mg, 60%, 10.2 mmol) in dioxane (8 mL) was added methyl 4-methyl-3-oxopentanoate (1.75 g, 12.2 mmol) at 0° C. under N₂ and the resulting mixture was stirred for 1 h. To the mixture was added DMF (16 mL) and a solution of F-3.12 (1.0 g, 2.05 mmol) in dioxane (8 mL) dropwise. The reaction mixture was stirred at 105° C. for 16 h. The mixture was poured into saturated aqueous NH₄Cl (50 mL) and the aqueous phase was extracted with EtOAc (2×50 mL). The combined organic layers were washed with water (2×50 mL), dried over anhydrous Na₂SO₄, filtered, and concentrated to give F-3.13 (1.0 g).

### Synthesis of F-3.14

To a solution of F-3.13 (1.0 g, 1.98 mmol) in MeOH (15 mL) was added H₂O (5 mL) and NaOH (787 mg, 19.7 mmol) at 25° C. and the resulting mixture was stirred at 60° C. for 16 h. The reaction mixture was concentrated, and H₂O (20 mL) was added. The mixture was extracted with EtOAc (3×30 mL), and the combined organic layers were washed with brine (15 mL), dried over anhydrous Na₂SO₄, filtered, and concentrated. The residue was purified by silica gel chromatography (0-10% of EtOAc in PE) to give F-3.14 (670 mg, 76.1%). ¹H NMR (400 MHz, CDCl₃) δ_{H} 2.67-2.55 (m, 1H), 2.51-2.28 (m, 2H), 2.26-2.18 (m, 1H), 1.84-1.59 (m, 5H), 1.54-1.29 (m, 10H), 1.29-1.10 (m, 8H), 1.10-1.07 (m, 6H), 1.07-0.98 (m, 4H), 0.96 (d, J=6.4 Hz, 3H), 0.94-0.91 (m, 1H), 0.88 (t, J=7.2 Hz, 6H), 0.81 (s, 3H), 0.67-0.58 (m, 1H). LC-ELSD/MS 30-90AB_2 min_E, purity>99%, MS ESI calcd. for C₃₀H₅₁O [M−H₂O+H]⁺ 427.4, found 427.4.

### Synthesis of F-3.15

To a solution of F-3.14 (600 mg, 1.34 mmol) in MeOH (15 mL) was added NaBH₄ (253 mg, 6.70 mmol) at 25° C. and the resulting mixture was stirred for 2 h. The reaction mixture was quenched with saturated aqueous NH₄Cl (100 mL) at 20° C. and the aqueous phase was extracted with EtOAc (2×20 mL). The combined organic layers were washed with brine (2×10 mL), dried over anhydrous Na₂SO₄, filtered, and concentrated under reduced pressure to give F-3.15 (600 mg). ¹H NMR (400 MHz, CDCl₃) δ_{H} 3.37-3.23 (m, 1H), 2.24 (d, J=12.8 Hz, 1H), 1.85-1.53 (m, 10H), 1.51-1.39 (m, 6H), 1.33-1.05 (m, 11H), 1.02-0.96 (m, 6H), 0.94-0.86 (m, 13H), 0.81 (s, 3H), 0.68-0.56 (m, 1H).

### Synthesis of F-3.16

To a solution of F-3.15 (600 mg, 1.34 mmol) and imidazole (228 mg, 3.35 mmol) in DCE (12 mL) was added TBDPSCI (736 mg, 2.68 mmol) at 25° C. under N₂ and the resulting mixture was stirred at 60° C. for 16 h. Water (20 mL, sat., aq.) was added and the aqueous phase was extracted with EtOAc (2×20 mL). The combined organic layers were washed with water (20 mL), dried over anhydrous Na₂SO₄, filtered, and concentrated. The residue was purified by silica gel chromatography (0-2-5% of EtOAc in PE) to give F-3.16 (800 mg, 87.1%). ¹H NMR (400 MHz, CDCl₃) δ_{H} 7.73-7.64 (m, 4H), 7.42-7.32 (m, 6H), 3.60-3.40 (m, 1H), 2.16 (d, J=12.4 Hz, 1H), 1.61-1.51 (m, 10H), 1.41-1.17 (m, 12H), 1.17-0.94 (m, 16H), 0.93-0.81 (m, 13H), 0.80 (s, 3H), 0.79-0.52 (m, 5H).

### Synthesis of F-3.17

To a solution of F-3.16 (540 mg, 0.788 mmol) in pyridine (8 mL) was added BzCl (0.901 mL, 7.88 mmol, 1.22 g/mL) and the resulting mixture was stirred at 25° C. for 16 h. The reaction mixture was poured into H₂O (50 mL) and the mixture was extracted with EtOAc (2×20 mL). The combined organic layers were washed with brine (50 mL), dried over anhydrous Na₂SO₄, filtered, and concentrated. The residue was purified by silica gel chromatography (0˜1% of EtOAc in PE) to give F-3.17 (350 mg, 56.3%).

### Synthesis of F-3.18

To a solution of F-3.17 (450 mg, 0.570 mmol) in THF (4 mL) was added TBAF (5.70 mL, 5.70 mmol, 1M in THF) at 25° C. and the resulting mixture was stirred for 16 h. The reaction mixture was quenched with H₂O (50 mL) at 25° C. and the aqueous phase was extracted with EtOAc (2×50 mL). The combined organic layers were washed with brine (50 mL×2), dried over anhydrous Na₂SO₄, filtered, and concentrated under reduced pressure. The residue was purified by silica gel chromatography (0-15% of EtOAc in PE) to give F-3.18 (170 mg, 54.3% yield).

### Synthesis of F-3.19 and F-3.20

F-3.18 (170 mg, 0.308 mmol) was separated by SFC (DAICEL CHIRALPAK IG (250 mm*30 mm, 10 um)). Mobile phase: A: CO₂ B: 0.1% NH₃H₂O MEOH; Gradient: from Gradient: from 45% to 45% of B; flow rate: 80 mL/min) to give F-3.20 (40 mg, 23.6% yield) and F-3.19 (20 mg, 11.8% yield).

F-3.19: ¹H NMR (400 MHz, CDCl₃) δ_{H} 8.04-7.91 (m, 2H), 7.56-7.48 (m, 1H), 7.47-7.36 (m, 2H), 3.36-3.25 (m, 1H), 2.32-2.20 (m, 1H), 2.15-2.02 (m, 4H), 1.97-1.63 (m, 6H), 1.53-1.02 (m, 18H), 0.99 (d, J=6.4 Hz, 3H), 0.97-0.81 (m, 18H), 0.72-0.58 (m, 1H).

F-3.20: ¹H NMR (400 MHz, CDCl₃) δ_{H} 8.06-7.90 (m, 2H), 7.57-7.48 (m, 1H), 7.45-7.34 (m, 2H), 3.31 (s, 1H), 2.35-2.21 (m, 1H), 2.16-2.00 (m, 4H), 1.96-1.61 (m, 6H), 1.52-1.07 (m, 18H), 0.98 (d, J=6.4 Hz, 3H), 0.94-0.85 (m, 18H), 0.73-0.59 (m, 1H).

### Synthesis of F-6

To a solution of F-3.19 (20 mg, 0.0363 mmol) in THF (2 mL) and MeOH (1 mL) was added LiOH (51.9 mg, 2.17 mmol) and H₂O (0.5 mL) and the resulting mixture was stirred at 50° C. for 16 h. The mixture was poured into H₂O (10 mL) and extracted with EtOAc (15 mL). The combined organic layers were washed brine (15 mL), dried over anhydrous Na₂SO₄, filtered, and concentrated under reduced pressure to give a residue. The residue was purified by silica gel chromatography (3˜15% of EA in PE) to give F-6 (11.7 mg, 72.2%). ¹H NMR (400 MHz, CDCl₃) δ_{H} 3.35-3.25 (m, 1H), 2.28-2.19 (m, 1H), 1.83-1.71 (m, 1H), 1.68-1.49 (m, 14H), 1.45-1.24 (m, 8H), 1.22-1.01 (m, 8H), 0.98 (d, J=6.8 Hz, 3H), 0.93-0.87 (m, 12H), 0.81 (s, 3H), 0.69-0.57 (m, 1H). LC-ELSD/MS 30-90AB 2 min_E, purity>99%, MS ESI calcd. for C₃₀H₅₁ [M−2H₂O+H]⁺ 411.4, found 411.4.

### Synthesis of F-7

To a solution of F-3.20 (40 mg, 0.0726 mmol) in THF (2 mL) and MeOH (1 mL) was added LiOH (104 mg, 4.35 mmol) and H₂O (0.5 mL) and the resulting mixture was stirred at 50° C. for 16 h. The reaction mixture poured into H₂O (10 mL) and extracted with EtOAc (3×15 mL). The combined organic layers were washed with brine (2×6 mL), dried over anhydrous Na₂SO₄, filtered, and concentrated under reduced pressure to give a residue. The residue was purified by silica gel chromatography (3˜15% EtOAc in PE) to give F-7 (25.0 mg, 77.1%). ¹H NMR (400 MHz, CDCl₃) δ_{H} 3.35-3.27 (m, 1H), 2.30-2.19 (m, 1H), 1.84-1.73 (m, 1H), 1.69-1.48 (m, 12H), 1.44-1.25 (m, 10H), 1.24-1.02 (m, 8H), 0.98 (d, J=6.4 Hz, 3H), 0.92-0.86 (m, 12H), 0.81 (s, 3H), 0.66-0.58 (m, 1H). LC-ELSD/MS 30-90AB_2 min_E, purity>99%, MS ESI calcd. for C₃₀H₅₁ [M−2H₂O+H]⁺ 411.4, found 411.4.

### Example 72: Synthesis of (3S,5S,8S,9S,10S,13R,14R,17R)-3-ethyl-17-((2R,5S)-5-hydroxy-6-methylheptan-2-yl)-10-methylhexadecahydro-1H-cyclopenta[a]phenanthren-3-ol (F-8) & (3S,5S,8S,9S,10S,13R,14R,17R)-3-ethyl-17-((2R,5R)-5-hydroxy-6-methylheptan-2-yl)-10-methylhexadecahydro-1H-cyclopenta[a]phenanthren-3-ol (F-9)

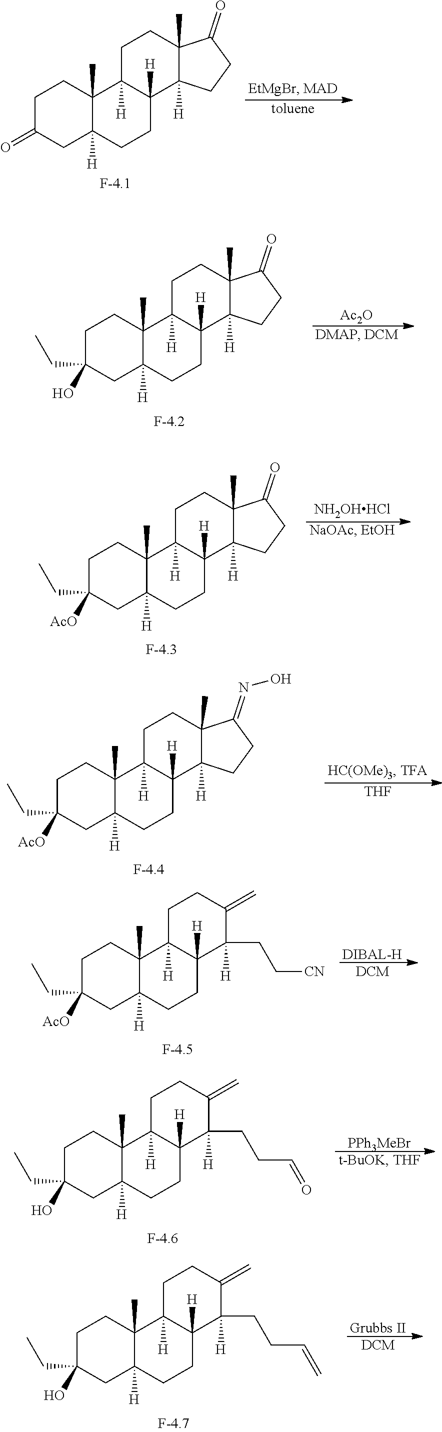

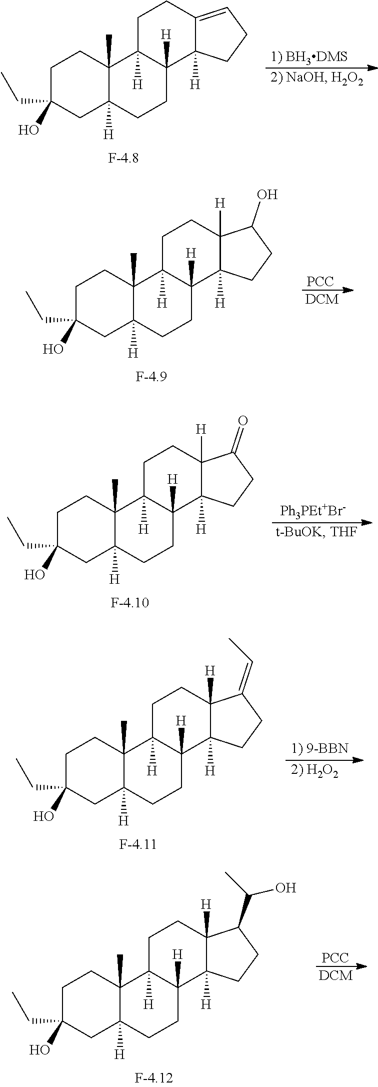

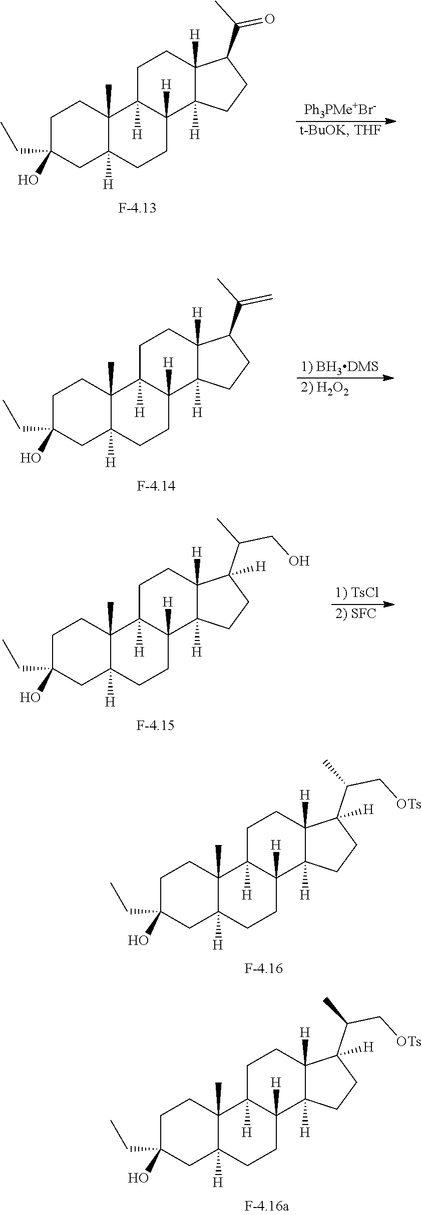

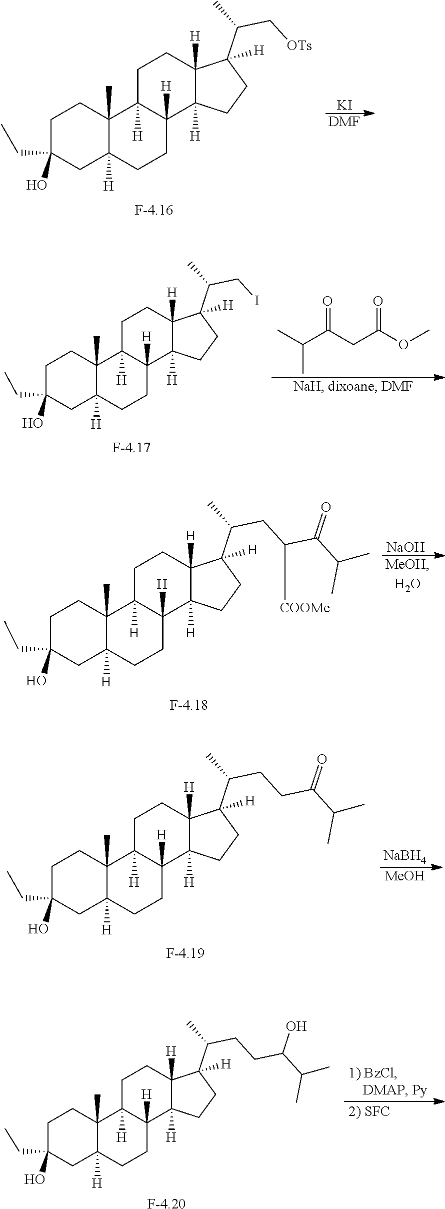

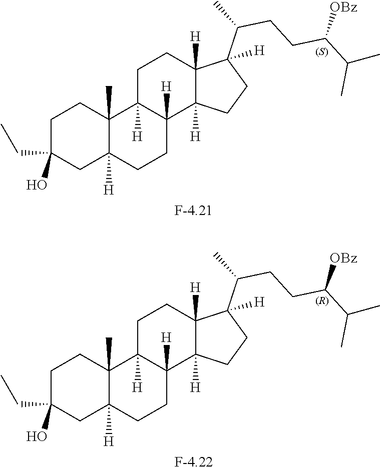

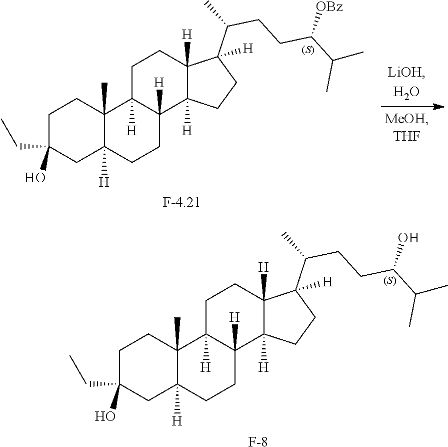

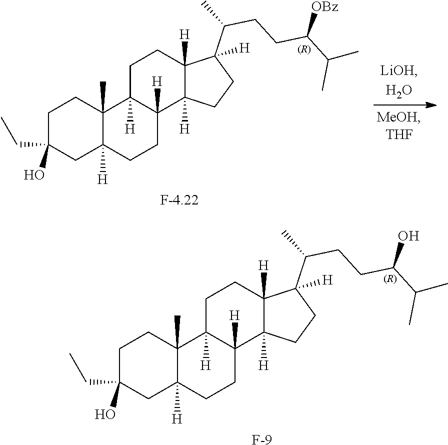

### Synthesis of F-4.2

To a solution of BHT (68.7 g, 312 mmol) in toluene (100 mL) under N₂ at 0° C. was added trimethylaluminum (2 M in toluene, 78 mL, 156 mmol) dropwise and the resulting mixture was stirred at 25° C. for 1 h. To the MAD solution was added a solution of F-4.1 (15 g, 52 mmol) in DCM (50 mL) dropwise at −70° C. and the mixture was stirred at −70° C. for 1 h under N₂. EtMgBr (52 mL, 156 mmol, 3M in ethyl ether) was added dropwise at -70° C. and the mixture was stirred for 2 h. The reaction mixture was poured into citric acid (500 mL, sat., aq.) at 10° C. and extracted with DCM (3×100 mL). The combined organic layers were washed with brine (2×50 mL), dried over anhydrous Na₂SO₄, filtered, and concentrated. The residue was triturated from MeOH (1.5 L) at 25° C. to give F-4.2 (14 g). ¹H NMR (400 MHz, CDCl₃) δ_{H} 2.50-2.38 (m, 1H), 2.12-2.00 (m, 1H), 1.97-1.88 (m, 1H), 1.83-1.74 (m, 2H), 1.70-1.48 (m, 9H), 1.41-1.33 (m, 3H), 1.29-1.23 (m, 3H), 1.21-1.07 (m, 2H), 1.05-0.94 (m, 2H), 0.90-0.81 (m, 9H), 0.78-0.68 (m, 1H).

### Synthesis of F-4.3

To a solution of F-4.2 (40 g, 125 mmol) in DCM (500 mL) was added TEA (37.9 g, 375 mmol), DMAP (15.2 g, 125 mmol) and Ac₂O (19 g, 187 mmol) and the resulting mixture was stirred at 15° C. for 16 h. The mixture was washed with NaHCO₃ (500 mL, sat. aq.), dried over anhydrous Na₂SO₄, filtered, and concentrated in vacuo to give F-4.3 (53 g). ¹H NMR (400 MHz, CDCl₃) δ_{H} 2.42 (dd, J=8.8, 19.2 Hz, 1H), 2.15-2.00 (m, 2H), 1.97 (s, 3H), 1.95-1.70 (m, 8H), 1.65-1.40 (m, 4H), 1.35-0.95 (m, 8H), 0.90-0.75 (m, 9H), 0.75-0.60 (m, 1H).

### Synthesis of F-4.4

To a suspension of F-4.3 (53 g, 147 mmol) in EtOH (500 mL) was added hydroxylamine hydrochloride (40.8 g, 588 mmol) and sodium acetate (48.2 g, 588 mmol) and the resulting mixture was refluxed at 80° C. for 2 h. The mixture was poured into water (1000 mL) and stirred for 20 minutes and filtered. The solid was washed with water (3×100 mL), dissolved in DCM (500 mL), dried over anhydrous Na₂SO₄, filtered, and concentrated to give F-4.4 (57 g).

### Synthesis of F-4.5

To a stirred solution of F-4.4 (47 g, 125 mmol) and CH(OMe)₃ (36.3 g, 343 mmol) in THF (500 mL) under N₂ at 60° C. was added TFA (17.7 g, 156 mmol) in one portion and the resulting mixture was stirred for 3 h. The mixture was quenched with NaHCO₃ (500 mL, sat. aq.) and extracted with EtOAc (500 mL). The mixture was filtered, and the organic layer was separated, concentrated, combined with another batch of 10 g F-4.5 and purified by silica gel chromatography (0˜10% EtOAc in PE) to give F-4.5 (16.5 g, 37%).

### Synthesis of F-4.6

To a solution of F-4.5 (16.5 g, 46.1 mmol) in DCM (200 mL) was added DIBAL-H (138 mL, 1 M in toluene, 138 mmol) at −70° C. and the resulting mixture was stirred at for 1 h. The mixture was poured into HCl (300 mL, 2 M) in portions. The organic layer was separated, dried over anhydrous Na₂SO₄, filtered, and concentrated in vacuo to give F-4.6 (17 g).

### Synthesis of F-4.7

To a mixture of MePPh₃Br (37.8 g, 106 mmol) in THF (120 mL) was added t-BuOK (11.8 g, 106 mmol) under N₂ and the resulting mixture was stirred at 40° C. for 30 min. F-4.6 (17 g, 53.3 mmol) in THF (30 mL) was added dropwise and the reaction mixture was stirred at 40° C. for 1 h. The reaction mixture was quenched with 10% aqueous NH₄Cl (100 mL) and extracted with EtOAc (200 mL). The organic layer was dried over anhydrous Na₂SO₄, filtered, concentrated under vacuum, triturated in MTBE (300 mL) and filtered to remove solid Ph₃PO. The filtrate was concentrated and purified by silica gel chromatography (0˜15% EtOAc in PE) to give F-4.7 (13.4 g).

### Synthesis of F-4.8

To a solution of F-4.7 (13.4 g, 42.3 mmol) in CH₂Cl₂ (1000 mL, anhydrous) was added Grubbs' catalyst second generation (CAS: 246047-72-3, 1.79 g, 2.11 mmol) under N₂ and the resulting mixture was stirred at 40° C. for 2 h. The mixture was washed with water (200 mL), dried over anhydrous Na₂SO₄, filtered, concentrated and purified by silica gel chromatography (0˜15% EtOAc in PE) to give F-4.8 (8.4 g, 69%).

### Synthesis of F-4.9

To a solution of F-4.8 (6.4 g, 22.1 mmol) in THF (60 mL) was added BH₃·Me₂S (6.63 mL, 10 M, 66.3 mmol) at 0° C. under N₂ and the resulting mixture was stirred at 20° C. for 16 h. To the mixture was added ethanol (20.3 g, 442 mmol), NaOH (44.2 mL, 5M aq., 221 mmol) and H₂O₂ (22.1 mL, 10 M, 221 mmol) dropwise and the mixture was stirred at 20° C. for 1 h. The mixture was quenched with Na₂SO₃ (100 mL, sat. aq.) and was concentrated to remove most organic solvent and filtered. The solid was washed with water (100 mL), re-dissolved in DCM (500 mL), dried over anhydrous Na₂SO₄, filtered, and concentrated in vacuo to give F-4.9 (7 g).

### Synthesis of F-4.10

To a suspension of F-4.9 (9 g, 29.3 mmol) in DCM (100 mL) was added silica gel (20 g) and PCC (9.43 g, 43.9 mmol) and the resulting mixture was stirred at 20° C. for 2 h. The mixture was concentrated and purified by silica gel chromatography (10˜30% EtOAc in PE) to give F-4.10 (7 g).

### Synthesis of F-4.11

To a suspension of EtPPh₃Br (17.0 g, 45.8 mmol) in THF (60 mL) was added t-BuOK (5.13 g, 45.8 mmol) under N₂ and the resulting mixture was stirred at 40° C. for 30 min. A solution of F-4.10 (7 g, 22.9 mmol) in THF (20 mL) was added dropwise and the mixture was stirred at 40° C. for 1 h. The reaction mixture was quenched with 10% aqueous NH₄Cl (50 mL) and was extracted with EtOAc (100 mL). The organic phase was concentrated, and the residue was triturated in MeOH/H₂O (1:1, 300 mL) to give F-4.11 (6.4 g, 88%).

### Synthesis of F-4.12

To a solution of F-4.11 (5.4 g, 3.15 mmol) in THF (50 mL) was added 9-BBN (170 mL, 0.5 M, 85 mmol) at 0° C. under N₂ and the resulting mixture was stirred at 50° C. for 16 h. To the mixture was added ethanol (39.1 g, 850 mmol), NaOH (68 mL, 5M aq., 340 mmol) and H₂O₂ (34 mL, 10 M, 340 mmol) dropwise and the mixture was stirred at 20° C. for 1 h. The mixture was quenched with Na₂SO₃ (500 mL, sat. aq.) and extracted with EOAc (2×200 mL). The combined organic layers were dried over anhydrous Na₂SO₄, filtered, concentrated, and purified by silica gel chromatography (0˜30% EtOAc in PE) to give F-4.12 (3 g).

### Synthesis of F-4.13

To a solution of F-4.12 (3 g, 8.96 mmol) in DCM (30 mL) was added silica gel (6 g) and PCC (2.88 g, 3.12 mmol) and the resulting mixture was stirred at 20° C. for 2 h. The mixture was concentrated and purified by silica gel chromatography (5˜25% EtOAc in PE) to give F-4.13 (3 g). ¹H NMR (400 MHz, CDCl₃) δ_{H} 2.55-2.40 (m, 1H), 2.13 (s, 3H), 1.95-1.75 (m, 6H), 1.70-1.60 (m, 4H), 1.50-1.25 (m, 4H), 1.25-0.95 (m, 11H), 0.88 (t, J=7.6 Hz, 3H), 0.79 (s, 3H), 0.75-0.65 (m, 1H). LC-ELSD/MS: purity>99%, MS ESI calcd. for C22H₃₅₀ [M+H−H₂O]⁺ 315.3, found 315.2.

### Synthesis of F-4.14

To a suspension of MePPh₃Br (8.57 g, 24.0 mmol) in THF (30 mL) was added t-BuOK (2.69 g, 24.0 mmol) under N₂ and the resulting mixture was stirred at 50° C. for 30 min. A solution of F-4.13 (4 g, 12.0 mmol) in THF (10 mL) was added dropwise and the reaction mixture was stirred at 40° C. for 1 h. The reaction mixture was quenched with 10% aqueous NH₄Cl (50 mL) and the mixture was extracted with EtOAc (100 mL). The organic phase was concentrated, and the residue was triturated in MeOH/H₂O (1:1, 120 mL) to give F-4.14 (4 g, 100%). ¹H NMR (400 MHz, CDCl₃) δ_{H} 4.70-4.65 (m, 2H), 2.15-2.00 (m, 1H), 1.90-1.70 (m, 5H), 1.66 (s, 3H), 1.60-1.35 (m, 9H), 1.20-0.95 (m, 10H), 0.95-0.85 (m, 4H), 0.80 (s, 3H), 0.75-0.65 (m, 1H).

### Synthesis of F-4.15

To a solution of F-4.14 (3.7 g, 11.1 mmol) in THF (50 mL) was added BH₃·Me₂S (3.32 mL, 10 M, 33.2 mmol) at 0° C. under N₂ and the resulting mixture was stirred at 20° C. for 16 h. To the mixture was added ethanol (10.2 g, 222 mmol), NaOH (22.2 mL, 5M aq., 111 mmol) and H₂O₂ (11.1 mL, 10 M, 111 mmol) dropwise and the mixture was stirred at 20° C. for 1 h. The mixture was quenched with Na₂SO₃ (50 mL, sat. aq.), combined with two other batches of 0.3 g of F-4.15, and concentrated to remove most of the organic solvent. The mixture was filtered and the solid was washed with water (50 mL), dissolved in DCM (500 mL), dried over anhydrous Na₂SO₄, filtered, and concentrated to give F-4.15 (4.4 g).

### Synthesis of F-4.16 and F-4.16a

To a solution of F-4.15 (4.4 g, 12.6 mmol) in DCM (75 mL) was added N-Me-imidazole (4.13 g, 50.4 mmol) and TsCl (3.60 g, 18.9 mmol) and the resulting mixture was stirred at 20° C. for 1 h. The mixture was quenched with saturated aqueous NH₄Cl (50 mL) and extracted with DCM (3×50 mL). The combined organic layers were washed with brine (2×50 mL), dried over anhydrous Na₂SO₄, filtered, and concentrated. The residue was purified by silica gel chromatography (0˜35% of EtOAc in PE) and separated by SFC (Column: DAICEL CHIRALPAK IG (250 mm*30 mm, 10 um); Condition: 0.1% NH₃H₂O ETOH; Begin B: 55%; End B: 55%; FlowRate (mL/min): 200; Injections: 350) to afford F-4.16 (3.2 g, 50%) and F-4.16a (1.4 g, 22%).

F-4.16: ¹H NMR (400 MHz, CDCl₃) δ_{H} 7.78 (d, J=8.4 Hz, 2H), 7.34 (d, J=8.0 Hz, 2H), 4.02-3.97 (m, 1H), 3.84-3.77 (m, 1H), 2.45 (s, 3H), 1.86-1.67 (m, 6H), 1.43-0.94 (m, 15H), 0.92-0.80 (m, 12H), 0.77 (s, 3H), 0.69-0.62 (m, 1H).

F-4.16a: ¹H NMR (400 MHz, CDCl₃) δ_{H} 7.78 (d, J=8.4 Hz, 2H), 7.34 (d, J=8.0 Hz, 2H), 3.90-3.79 (m, 2H), 2.45 (s, 3H), 1.99-1.91 (m, 1H), 1.78-1.65 (m, 5H), 1.45-0.92 (m, 17H), 0.91-0.82 (m, 7H), 0.79-0.76 (m, 6H), 0.72-0.66 (m, 1H).

### Synthesis of F-4.17

To a solution of F-4.16 (1.7 g, 3.38 mmol) in DMF (20 mL) was added KI (2.68 g, 16.2 mmol) at 20° C. and the resulting mixture was stirred at 60° C. for 16 h. The reaction mixture was poured into water (50 mL) and extracted with EtOAc (2×40 mL). The combined organic layers were washed with brine (20 mL), dried over anhydrous Na₂SO₄, filtered, and concentrated in vacuo. The residue was dissolved in EtOAc (40 mL), washed with LiCl (3×100 mL, 5% aq.), brine (20 mL), dried over anhydrous Na₂SO₄, filtered, and concentrated in vacuo to give F-4.17 (1.5 g, 97%). ¹H NMR (400 MHz, CDCl₃) δ_{H} 3.31 (dd, J=3.6, 9.6 Hz, 1H), 3.31 (dd, J=8.4, 9.2 Hz, 1H), 2.00-1.93 (m, 1H), 1.83-1.65 (m, 5H), 1.60-1.43 (m, 6H), 1.40-1.30 (m, 4H), 1.30-1.15 (m, 4H), 1.15-1.00 (m, 4H), 1.00-0.90 (m, 5H), 0.90-0.85 (m, 4H), 0.78 (s, 3H), 0.75-0.67 (m, 1H).

### Synthesis of F-4.18

To a solution of NaH (455 mg, 60%, 11.4 mmol) in dioxane (5 mL) was added methyl 4-methyl-3-oxopentanoate (2.10 g, 14.6 mmol) at 0° C. under N₂ and the resulting mixture was stirred at 0° C. for 1 h. DMF (20 mL) and F-4.17 (750 mg, 1.63 mmol) in dioxane (5 mL) were added dropwise and the reaction mixture was stirred at 105° C. for 16 h. The mixture was poured into saturated aqueous NH₄Cl (30 mL), and the aqueous phase was extracted with DCM (3×20 mL). The combined organic layers were washed with water (2×50 mL), dried over anhydrous Na₂SO₄, filtered, and concentrated to give F-4.18 (1.50 g).

### Synthesis of F-4.19

To a solution of F-4.18 (1.50 g, 3.15 mmol) in MeOH (20 mL) was added NaOH (1.25 g, 31.5 mmol) in H₂O (2 mL) at 25° C. and the resulting mixture was stirred at 60° C. for 70 h. The reaction mixture was concentrated, and H₂O (50 mL) was added. The mixture was extracted with EtOAc (3×30 mL) and the combined organic layers were washed with brine (30 mL), dried over anhydrous Na₂SO₄, filtered, and concentrated. The residue was purified by silica gel chromatography (0-20% of EtOAc in PE) to give F-4.19 (820 mg, 55%). 1H NMR (400 MHz, CDCl₃) δ_{H} 2.65-2.56 (m, 1H), 2.54-2.44 (m, 1H), 2.41-2.32 (m, 1H), 1.96-1.86 (m, 1H), 1.80-1.59 (m, 8H), 1.56-1.16 (m, 12H), 1.15-1.11 (m, 1H), 1.10 (s, 3H), 1.08 (s, 3H), 1.04-0.84 (m, 13H), 0.78 (s, 3H), 0.73-0.66 (m, 1H). LC-ELSD/MS 30-90AB_2 min_E, purity 99%, MS ESI calcd. for C28H₄₇₀ [M−H₂O+H]⁺ 399.4, found 399.4.

### Synthesis of F-4.20

To a solution of F-4.19 (770 mg, 1.84 mmol) in MeOH (5 mL) was added NaBH₄ (69.6 mg, 1.84 mmol) at 25° C. under N₂ and the resulting mixture was stirred for 1 h. The reaction mixture was quenched with saturated aqueous NH₄Cl (20 mL) at 25° C. and the aqueous phase was extracted with EtOAc (2×30 mL). The combined organic layers were washed with brine (30 mL), dried over anhydrous Na₂SO₄, filtered, and concentrated to give F-4.20 (370 mg).

### Synthesis of F-4.21 and 4.22

To a solution of F-4.20 (370 mg, 0.884 mmol) and DMAP (107 mg, 0.883 mmol) in pyridine (5 mL) was added benzoyl chloride (1.24 g, 8.83 mmol) dropwise and the resulting mixture was stirred at 25° C. for 24 h. Water (0.1 mL) was added, and the mixture was stirred at 25° C. for 16 h. The mixture was quenched with water (30 mL) and extracted with EtOAc (3×20 mL). The combined organic layers were washed with brine (30 mL), dried over anhydrous Na₂SO₄, filtered, and concentrated. The residue was purified by silica gel chromatography (0˜20% of EtOAc in PE) and separated by SFC (Column: DAICEL CHIRALPAK AD (250 mm*30 mm, 10 um); Condition: 0.1% NH₃H₂O IPA; Begin B: 35%; End B: 35%; FlowRate (ml/min): 70; Injections: 60) to afford F-4.21 (120 mg, 25%) and F-4.22 (120 mg, 25%).

F-4.21: ¹H NMR (400 MHz, CDCl₃) δ_{H} 8.08-8.01 (m, 2H), 7.58-7.49 (m, 1H), 7.48-7.38 (m, 2H), 5.01-4.92 (m, 1H), 2.02-1.92 (m, 1H), 1.90-1.82 (m, 1H), 1.78-1.58 (m, 8H), 1.53-1.01 (m, 15H), 1.00-0.94 (m, 7H), 0.94-0.80 (m, 12H), 0.75 (s, 3H), 0.69-0.61 (m, 1H).

F-4.22: ¹H NMR (400 MHz, CDCl₃) δ_{H} 8.10-8.00 (m, 2H), 7.61-7.51 (m, 1H), 7.49-7.37 (m, 2H), 5.02-4.93 (m, 1H), 2.02-1.90 (m, 1H), 1.88-1.81 (m, 1H), 1.79-1.63 (m, 4H), 1.54-1.25 (m, 9H), 1.24-1.01 (m, 10H), 0.99-0.94 (m, 7H), 0.94-0.80 (m, 12H), 0.74 (s, 3H), 0.69-0.59 (m, 1H).

### Synthesis of F-8

To a solution of F-4.21 (120 mg, 0.230 mmol) in THF (1 mL) was added MeOH (2 mL), H₂O (1 mL) and LiOH (109 mg, 4.59 mmol) and the resulting mixture was stirred at 25° C. for 72 h. The mixture was diluted with water (10 mL) and extracted with EtOAc (3×10 mL). The combined organic layers were dried over anhydrous Na₂SO₄, filtered, and concentrated. The residue was purified by silica gel chromatography (0˜30% of EtOAc in PE) to give F-8 (64.4 mg, 54%). ¹H NMR (400 MHz, CDCl₃) δ_{H} 3.37-3.29 (m, 1H), 1.96-1.87 (m, 1H), 1.81-1.57 (m, 9H), 1.50-1.06 (m, 14H), 1.05-0.95 (m, 4H), 0.95-0.90 (m, 7H), 0.90-0.81 (m, 10H), 0.79 (s, 3H), 0.75-0.68 (m, 1H). LC-ELSD/MS 30-90AB_2 min_E, purity 99%, MS ESI calcd. for C28H₄₇ [M−2H₂O+H]⁺ 383.3, found 383.3.

### Synthesis of F-9

To a solution of F-4.22 (120 mg, 0.230 mmol) in THF (1 mL) was added MeOH (2 mL), H₂O (1 mL) and LiOH (109 mg, 4.59 mmol) and the resulting mixture was stirred at 25° C. for 72 h. The mixture was diluted with water (10 mL) and extracted with EtOAc (3×10 mL). The combined organic layers were dried over anhydrous Na₂SO₄, filtered, and concentrated. The residue was purified by silica gel chromatography (0˜30% of EtOAc in PE) to give F-9 (56.9 mg, 60%). ¹H NMR (400 MHz, CDCl₃) δ_{H} 3.38-3.28 (m, 1H), 1.98-1.84 (m, 1H), 1.82-1.56 (m, 9H), 1.53-1.17 (m, 14H), 1.16-0.95 (m, 5H), 0.95-0.89 (m, 10H), 0.89-0.85 (m, 6H), 0.79 (s, 3H), 0.73-0.66 (m, 1H). LC-ELSD/MS 30-90AB_2 min_E, purity 99%, MS ESI calcd. for C28H₄₇ [M−2H₂O+H]⁺ 383.3, found 383.3.

### Example 73: Synthesis of (3S,8S,9S,10R,13R,14R,17R)-3-ethyl-17-((2R,5R)-5-hydroxy-6-methylheptan-2-yl)-10-methyl-2,3,4,7,8,9,10,11,12,13,14,15,16,17-tetradecahydro-1H-cyclopenta[a]phenanthren-3-ol (F-10)

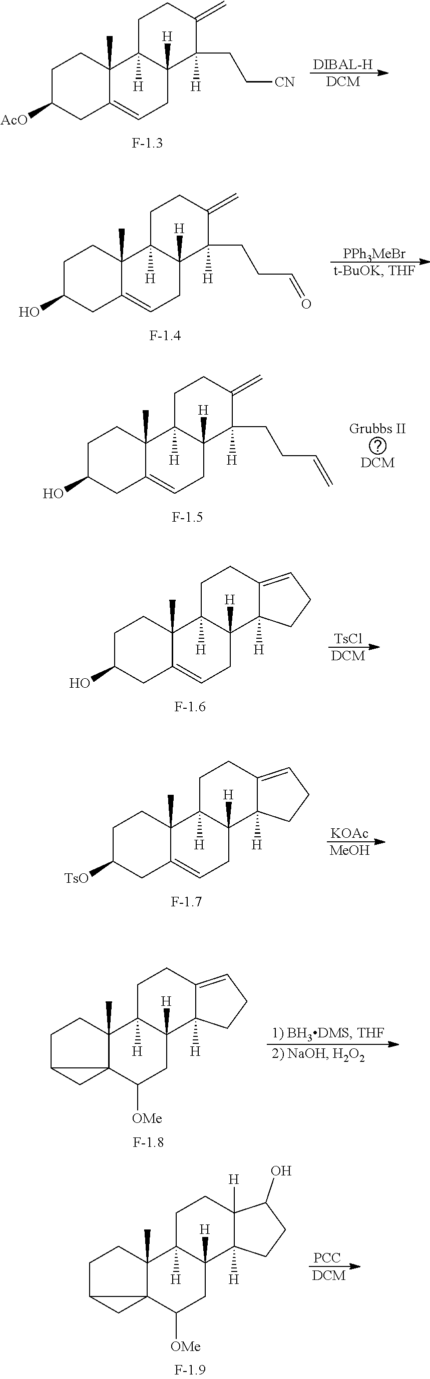

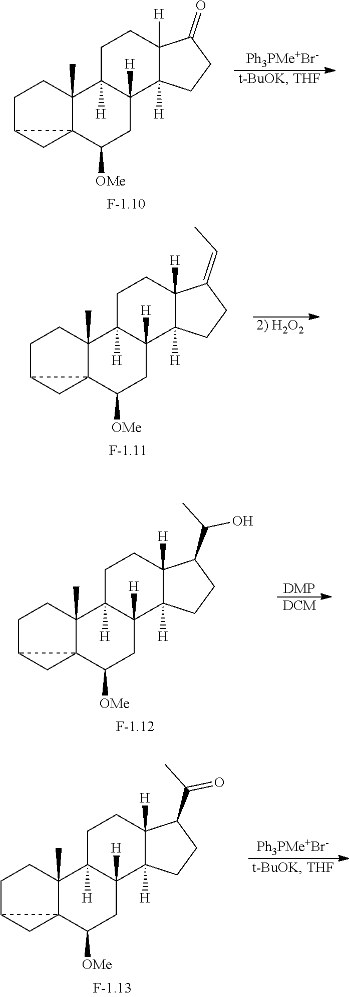

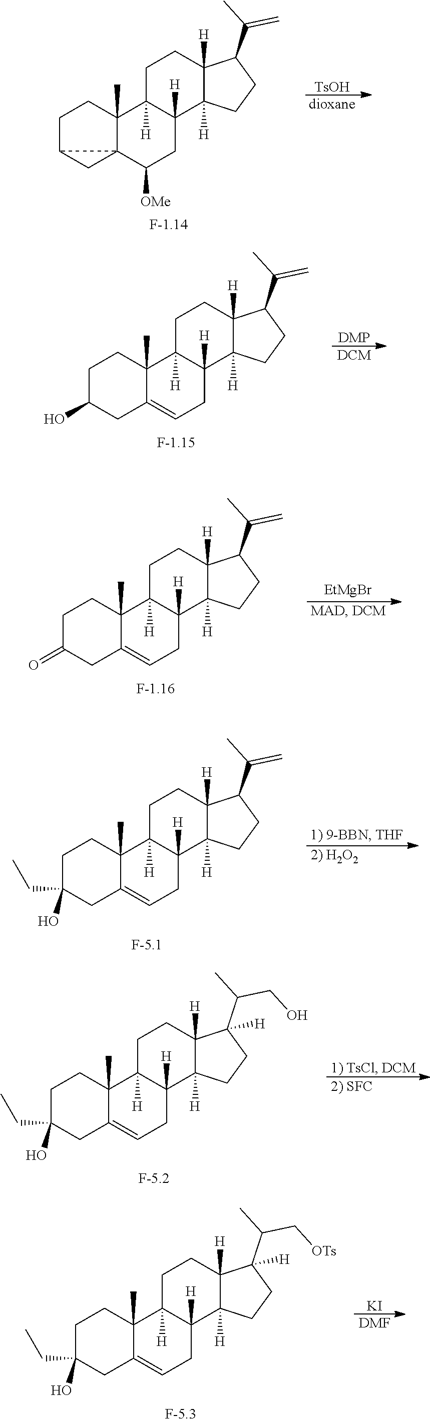

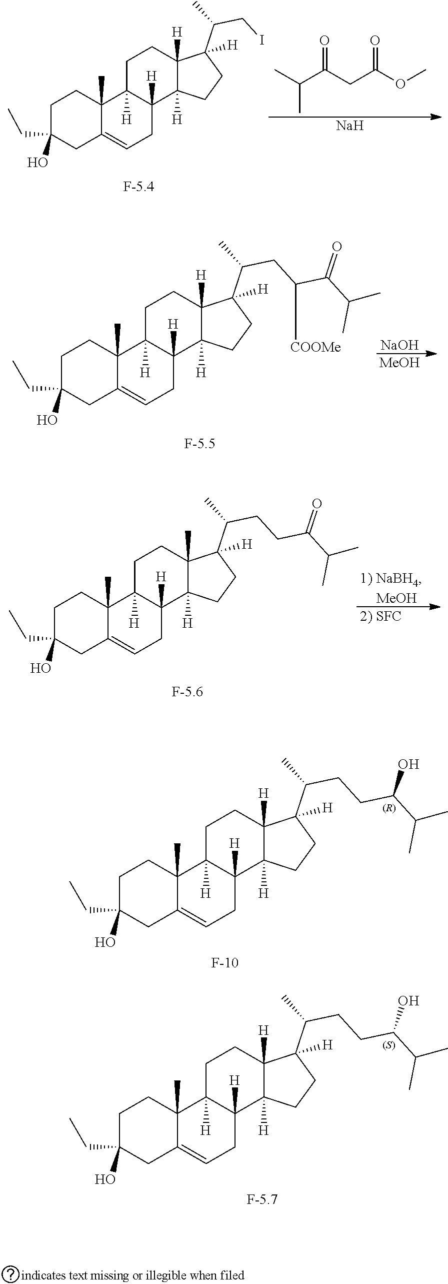

### Synthesis of F-1.4

To a solution of F-1.3 (20 g, 61.0 mmol; preparation described in J. Org. Chem. 2003, 68, 4579-4581) in DCM (200 mL) was added DIBAL-H (183 mL, 1 M in toluene, 183 mmol) dropwise at −70° C. under N₂ and the resulting mixture was stirred at for 1 h. HCl (500 mL, 2 M) was added dropwise into the mixture. The organic layer was separated, dried over anhydrous Na₂SO₄, filtered, and concentrated in vacuo to give F-1.4 (13 g), which was used directly in the next step.

### Synthesis of F-1.5

To a solution of MePPh₃Br (35.3 g, 99 mmol) in THF (300 mL) was added t-BuOK (11.1 g, 99 mmol) under N₂ and the resulting mixture was stirred at 40° C. for 30 min. F-1.4 (14.3 g, 49.5 mmol) in THF (150 mL) was added dropwise and the mixture was stirred at 40° C. for 1 h. The reaction mixture was quenched with 10% aqueous NH₄Cl (500 mL) and extracted with EtOAc (2×500 mL). The combined organic layers were dried over anhydrous Na₂SO₄, filtered, and concentrated. The residue was triturated in MTBE (500 mL) to remove solid Ph₃PO. The filtrate was concentrated and purified by silica gel chromatography (0˜20% EtOAc in PE) to give F-1.5 (7 g, 37% yield over two steps). ¹H NMR (400 MHz, CDCl₃) δ_{H} 5.91-5.73 (m, 1H), 5.45-5.30 (m, 1H), 5.03-4.89 (m, 2H), 4.76 (s, 1H), 4.58 (s, 1H), 3.60-3.47 (m, 1H), 2.40-2.11 (m, 5H), 2.02-1.80 (m, 5H), 1.73-1.43 (m, 7H), 1.23-1.04 (m, 3H), 0.98-0.90 (m, 3H).

### Synthesis of F-1.6

To a solution of F-1.5 (2 g, 6.98 mmol) in CH₂Cl₂ (80 mL, anhydrous) was added Grubbs' catalyst second generation (CAS: 246047-72-3, 296 mg, 0.35 mmol) under N₂ and the resulting mixture was stirred at 40° C. for 2 h. The mixture was washed with water (20 mL) and the organic layer was dried over anhydrous Na₂SO₄, filtered, concentrated and purified by silica gel chromatography (0˜20% EtOAc in PE) to give F-1.6 (1.7 g, 94.4% yield). ¹H NMR (400 MHz, CDCl₃) δ_{H} 5.41-5.35 (m, 1H), 5.25-5.20 (m, 1H), 3.63-3.48 (m, 1H), 2.57-2.45 (m, 1H), 2.34-2.19 (m, 4H), 2.19-1.95 (m, 3H), 1.75-1.57 (m, 3H), 1.54-1.35 (m, 3H), 1.32-0.99 (m, 5H), 0.93 (s, 3H), 0.90-0.78 (m, 1H).

### Synthesis of F-1.7

To a solution of F-1.6 (1.7 g, 6.57 mmol) in DCM (15 mL) was added 1-methyl-1H-imidazole (1.61 mg, 19.7 mmol) and TsCl (1.87 mg, 9.85 mmol) at 20° C. and the resulting mixture was stirred for 2 h. The mixture was poured into H₂O (70 mL) and extracted with DCM (3×70 mL). The combined organic layers were washed with saturated aqueous NaHCO₃ (70 mL), HCl (11 mL, 0.5M), brine (70 mL), dried over anhydrous Na₂SO₄, filtered, and concentrated to give F-1.7 (2.35 g), which was used directly in the next step.

### Synthesis of F-1.8

To a suspension of F-1.7 (2.35 g, 5.69 mmol) in MeOH (90 mL) was added KOAc (2.78 g, 28.4 mmol) and the resulting mixture was then heated to reflux at 65° C. for 2 h. The mixture was cooled and concentrated in vacuo. Water (20 mL) was added, and the mixture was extracted with petroleum ether (100 mL). The organic layer was washed with brine (100 mL), dried over anhydrous Na₂SO₄, filtered, and concentrated in vacuo to give F-1.8 (1.7 g), which was used directly in the next step.

### Synthesis of F-1.9

To a solution of F-1.8 (16.3 g, 59.8 mmol) in THF (240 mL) was added BH₃·Me₂S (11.9 mL, 10 M, 119 mmol) and the resulting mixture was stirred at 15° C. for 16 h. To the mixture was added EtOH (34.3 mL, 598 mmol) dropwise followed by NaOH (119 mL, 598 mmol, 5 M in water) and H₂O₂ (59.8 mL, 10 M, 598 mmol). After stirring at 78° C. for 2 h, the mixture was quenched with Na₂S₂O₃ (200 mL, 10% aqueous) and extracted with EtOAc (2×200 mL). The combined organic layers were washed with brine (2×200 mL), dried over anhydrous Na₂SO₄, filtered, and concentrated in vacuo to give F-1.9 (17.0 g), which was used directly in the next step.

### Synthesis of F-1.10

To a solution of F-1.9 (16.5 g, 56.8 mmol) in DCM (160 mL) at 0° C. was added silica gel (24 g) and PCC (24.2 g, 113 mmol) and the resulting mixture was stirred at 10° C. for 1 h. The mixture was filtered through a pad of silica gel and the filter cake was washed with DCM (3×50 mL). The filtrate was concentrated to give a residue that was purified by silica gel chromatography (0˜10% EtOAc in PE) to give F-1.10 (6.0 g, 35% yield over four steps). ¹H NMR (400 MHz, CDCl₃) δ_{H} 3.40-3.27 (m, 3H), 2.88-2.75 (m, 1H), 2.31-2.25 (m, 2H), 2.20-2.07 (m, 2H), 2.04-1.64 (m, 5H), 1.52-1.09 (m, 6H), 1.06-0.85 (m, 6H), 0.84-0.78 (m, 1H), 0.72-0.62 (m, 1H), 0.51-0.40 (m, 1H).

### Synthesis of F-1.11

To a solution of EtPPh₃Br (15.8 g, 42.8 mmol) in THF (70 mL) was added t-BuOK (4.79 g, 42.8 mmol) at 15° C. under N₂ and the resulting mixture was stirred at 40° C. for 60 min. F-1.10 (6.2 g, 21.4 mmol) was added in portions to keep the internal temperature below 40° C. After stirring at 40° C. for 16 h, the reaction mixture was quenched with 10% aqueous NH₄Cl (70 mL) at 15° C. and the aqueous phase was extracted with EtOAc (70 mL). The combined organic layers were washed with 10% aqueous NH₄Cl, dried over anhydrous Na₂SO₄, filtered, and concentrated under vacuum to give a residue, which was purified by trituration MeOH: H2O=1:1 (200 mL) to give F-1.11 (7.0 g), which was used directly in the next step.

### Synthesis of F-1.12

To a solution of F-1.11 (7.5 g, 24.9 mmol) in THF (75 mL) was added 9-BBN dimer (12.0 g, 49.8 mmol) and the resulting mixture was stirred at 15° C. for 16 h. To the mixture was added EtOH (17.1 mL, 298 mmol) dropwise followed by NaOH (59.6 mL, 5 M, 298 mmol) and H₂O₂ (29.8 mL, 10 M, 298 mmol). The mixture was stirred at 78° C. for 2 h. The mixture was quenched with Na₂S₂SO₃ (150 mL, 30% aqueous) and extracted with EtOAc (150 mL). The organic layer was separated and washed with saturated aqueous NaHCO₃/Na₂S₂O₃ (1:1, 2×100 mL), brine (100 mL), dried over anhydrous Na₂SO₄, filtered, and concentrated to give a residue which was purified by silica gel chromatography (0˜10% EtOAc in PE) to give F-1.12 (6.3 g, 79% yield over two steps). ¹H NMR (400 MHz, CDCl₃) δ_{H} 3.94-3.64 (m, 1H), 3.40-3.24 (m, 3H), 2.85-2.71 (m, 1H), 2.21-1.91 (m, 2H), 1.87-1.64 (m, 6H), 1.51-1.02 (m, 13H), 0.99 (s, 3H), 0.93-0.81 (m, 3H), 0.65 (t, J=3.6 Hz, 1H), 0.43 (dd, J=5.6, 8.4 Hz, 1H).

### Synthesis of F-1.13

To a solution of F-1.12 (6.1 g, 19.1 mmol) in DCM (68 mL) was added DMP (16.1 g, 38.2 mmol) and the resulting mixture was stirred at 40° C. for 0.5 h. The mixture was quenched with saturated aqueous NaHCO₃ (100 mL) at 10° C. and extracted with DCM (50 mL). The DCM phase was separated and washed with saturated aqueous NaHCO₃/Na₂S₂O₃ (1:1, 2×100 mL), brine (100 mL), dried over anhydrous Na₂SO₄, filtered, and concentrated to give a residue which was purified by silica gel chromatography (0˜15% EtOAc in PE) to give F-1.13 (5.9 g, 97% yield). ¹H NMR (400 MHz, CDCl₃) δ_{H} 3.32 (s, 3H), 2.82-2.70 (m, 1H), 2.57-2.34 (m, 1H), 2.14 (s, 3H), 2.05-1.64 (m, 8H), 1.52-1.02 (m, 8H), 0.99 (s, 3H), 0.94-0.81 (m, 3H), 0.71-0.55 (m, 1H), 0.50-0.34 (m, 1H).

### Synthesis of F-1.14

To a solution of MePPh₃Br (13.2 g, 37.2 mmol) in THF (50 mL) was added t-BuOK (4.17 g, 37.2 mmol) at 15° C. under N₂ and the resulting mixture was stirred at 40° C. for 60 min. F-1.13 (5.9 g, 18.6 mmol) was added in portions to keep the internal temperature below 40° C. The reaction mixture was stirred at 50° C. for 16 h. The reaction mixture was quenched with 10% aqueous NH₄Cl (50 mL) at 15° C. and the aqueous phase was extracted with EtOAc (50 mL). The combined organic layers were washed with 10% aqueous NH₄Cl, dried over anhydrous Na₂SO₄, filtered, and concentrated. The residue was purified by trituration MeOH: H₂O=1:1 (180 mL) to give the F-1.14 (5.8 g, 98% yield). ¹H NMR (400 MHz, CDCl₃) δ_{H} 4.72-4.60 (m, 2H), 3.33 (s, 3H), 2.85-2.71 (m, 1H), 2.15-1.96 (m, 2H), 1.92-1.69 (m, 5H), 1.67 (s, 3H), 1.58-1.54 (m, 2H), 1.52-1.46 (m, 2H), 1.28-1.08 (m, 4H), 1.00 (s, 3H), 0.97-0.77 (m, 5H), 0.65 (t, J=4.4 Hz, 1H), 0.44 (dd, J=5.2, 8.0 Hz, 1H).

### Synthesis of F-1.15

To a solution of F-1.14 (5.3 g, 16.8 mmol) in dioxane (50 mL) was added a solution of TsOH (319 mg, 1.68 mmol) in water (15 mL) and the resulting mixture was stirred at 75° C. for 16 h. To the mixture was added NaHCO₃ (50 mL, sat., aq.) and the mixture was extracted with EtOAc (50 mL). The organic layer was separated, dried over anhydrous Na₂SO₄, filtered, and concentrated in vacuo. The residue was purified by silica gel chromatography (20˜33% EtOAc in PE) to give F-1.15 (3.5 g, 69% yield). ¹H NMR (400 MHz, CDCl₃) δ_{H} 5.40-5.32 (m, 1H), 4.68 (s, 2H), 3.63-3.45 (m, 1H), 2.35-2.17 (m, 2H), 2.15-2.01 (m, 2H), 1.93-1.75 (m, 6H), 1.68 (s, 3H), 1.63-1.47 (m, 5H), 1.35-1.00 (m, 6H), 0.97 (s, 3H), 0.94-0.85 (m, 1H).

### Synthesis of F-1.16

To a solution of F-1.15 (3.5 g, 11.6 mmol) in DCM (70 mL) was added DMP (9.83 g, 23.2 mmol) and the resulting mixture was stirred at 20° C. for 0.5 h. The mixture was quenched with saturated aqueous NaHCO₃ (100 mL) at 10° C. and extracted with DCM (100 mL). The DCM phase was separated and washed with saturated aqueous NaHCO₃/Na₂S₂O₃ (1:1, 2×50 mL), brine (100 mL), dried over anhydrous Na₂SO₄, filtered, and concentrated to give F-1.16 (3.5 g), which was used directly in the next step.

### Synthesis of F-5.1

To a solution of BHT (15.4 g, 70.2 mmol) in toluene (30 mL) under nitrogen at 0° C. was added AlMe₃ (2 M in toluene, 17.5 mL, 35.1 mmol) dropwise and the resulting mixture was stirred at 20° C. for 1 h. To the freshly prepared MAD (35.1 mmol, in 30 mL toluene) solution was added F-1.16 (3.5 g, 11.7 mmol) in DCM (40 mL) dropwise at −70° C. After stirring at -70° C. for 1 h under N₂, EtBrMg (19.5 mL, 58.5 mmol, 3M in ethyl ether) was added dropwise. After stirring at -70° C. for 1 h, the reaction mixture was poured into citric acid (100 mL, 20% aqueous) at 10° C. and extracted with EtOAc (2×60 mL). The combined organic layers were washed with brine (2×60 mL), dried over anhydrous Na₂SO₄, filtered, and concentrated in vacuo. The residue was purified by silica gel chromatography (0˜15% EtOAc in PE) to give F-5.1 (2.0 g, 52% yield over two steps). ¹H NMR (400 MHz, CDCl₃) δ_{H} 5.39-5.20 (m, 1H), 4.70-4.67 (m, 2H), 2.41-2.33 (m, 2H), 2.08-2.02 (m, 4H), 1.80-1.72 (m, 4H), 1.68 (s, 3H), 1.61-1.35 (m, 7H), 1.30-1.23 (m, 2H), 1.14-1.08 (m, 4H), 0.99 (s, 3H), 0.89-0.81 (m, 4H).

### Synthesis of F-5.2

To a solution of F-5.1 (2.1 g, 6.39 mmol) in THF (20 mL) was added 9-BBN dimer (3.07 g, 12.7 mmol) and the resulting mixture was stirred at 15° C. for 16 h. To the mixture was added EtOH (4.40 mL, 76.6 mmol) dropwise followed by NaOH (15.3 mL, 5 M, 76.6 mmol) and H₂O₂ (7.66 mL, 10 M, 76.6 mmol) and the mixture was stirred at 78° C. for 2 h. The mixture was quenched with Na₂S₂SO₃ (50 mL, 30% aqueous) and extracted with EtOAc (50 mL). The organic layer was separated and washed with saturated aqueous NaHCO₃/Na₂S₂O₃ (1:1, 2×50 mL), brine (50 mL), dried over anhydrous Na₂SO₄, filtered, and concentrated to give a residue, which was purified by silica gel chromatography (0˜35% EtOAc in PE) to give F-5.2 (1.26 g, 57% yield). ¹H NMR (400 MHz, CDCl₃) δ_{H} 5.32-5.22 (m, 1H), 3.69-3.60 (m, 1H), 3.49-3.37 (m, 1H), 2.42-2.31 (m, 1H), 2.13-1.94 (m, 4H), 1.83-1.53 (m, 11H), 1.50-1.16 (m, 10H), 1.00-0.96 (m, 6H), 0.87-0.83 (m, 3H).

### Synthesis of F-5.3

To a solution of F-5.2 (1.0 g, 2.88 mmol) in DCM (20 mL) was added 1-methyl-1H-imidazole (707 mg, 8.63 mmol) and TsCl (821 mg, 4.31 mmol) at 20° C. and the resulting mixture was stirred for 16 h. The mixture was poured into H₂O (30 mL) and extracted with DCM (3×10 mL). The combined organic layers were washed with saturated aqueous NaHCO₃ (20 mL), 0.5 M HCl (20 mL), brine (20 mL), dried over anhydrous Na₂SO₄, filtered, and concentrated. The residue was separated by SFC (DAICEL CHIRALPAK AD (250 mm*30 mm, 10 um)). Mobile phase: A: CO₂ B: 0.1% NH₃H₂O EtOH; Gradient: from 35% to 35% of B; flow rate: 70 mL/min) to give F-5.3 (720 mg, 50% yield). ¹H NMR (400 MHz, CDCl₃) δ_{H} 7.79 (d, J=8.4 Hz, 2H), 7.34 (d, J=8.0 Hz, 2H), 5.31-5.22 (m, 1H), 4.01 (dd, J=4.8, 9.6 Hz, 1H), 3.82 (dd, J=7.6, 9.2 Hz, 1H), 2.45 (s, 3H), 2.39-2.32 (m, 1H), 2.10-2.05 (m, 1H), 2.04-1.99 (m, 1H), 1.90-1.56 (m, 10H), 1.55-1.29 (m, 5H), 1.23-0.98 (m, 6H), 0.97 (s, 3H), 0.93 (d, J=6.8 Hz, 3H), 0.91-0.88 (m, 1H), 0.85 (t, J=7.6 Hz, 3H).

### Synthesis of F-5.4

To a solution of F-5.3 (720 mg, 1.43 mmol) in DMF (14 mL) was added KI (1.18 g, 7.15 mmol) and the resulting mixture was stirred at 60° C. for 1 h. The mixture was cooled and poured into water (30 mL) and stirred for 5 min. The aqueous phase was extracted with EtOAc (2×30 mL). The combined organic layers were washed with brine (2×30 mL), dried over anhydrous Na₂SO₄, filtered, and concentrated to give F-5.4 (550 mg, 84% yield). 1H NMR (400 MHz, CDCl₃) δ_{H} 5.32-5.24 (m, 1H), 3.33 (dd, J=3.6, 9.6 Hz, 1H), 3.10 (dd, J=8.0, 9.6 Hz, 1H), 2.41-2.30 (m, 1H), 2.13-2.05 (m, 2H), 2.03-1.99 (m, 1H), 1.81-1.71 (m, 4H), 1.67-1.33 (m, 11H), 1.22-1.08 (m, 3H), 1.05 (d, J=6.4 Hz, 3H), 1.04-1.00 (m, 2H), 0.99 (s, 3H), 0.95-0.90 (m, 1H), 0.85 (t, J=7.6 Hz, 3H).

### Synthesis of F-5.5

To a solution of NaH (153 mg, 60%, 3.93 mmol) in dioxane (7 mL) was added methyl 4-methyl-3-oxopentanoate (772 mg, 5.36 mmol) at 0° C. under N₂ and the resulting mixture was stirred at 0° C. for 1 h. DMF (2 mL) and a solution of F-5.4 (350 mg, 0.766 mmol) in DMF (4 mL) were added dropwise. The reaction mixture was stirred at 105° C. for 16 h. The mixture was cooled and poured into saturated aqueous NH₄Cl (50 mL) and the aqueous phase was extracted with EtOAc (2×50 mL). The combined organic layers were washed with water (2×50 mL), dried over anhydrous Na₂SO₄, filtered, and concentrated to give F-5.5 (350 mg), which was used directly in the next step.

### Synthesis of F-5.6

To a solution of F-5.5 (350 mg, 0.740 mmol) in MeOH (7 mL) was added H₂O (2.5 mL) and NaOH (295 mg, 7.40 mmol) at 25° C. and the resulting mixture was stirred at 60° C. for 16 h. H₂O (30 mL) was added to the reaction and the mixture was extracted with EtOAc (2×20 mL). The combined organic layers were washed with brine (20 mL), dried over anhydrous Na₂SO₄, filtered, and concentrated. The residue was purified by silica gel chromatography (0-15% EtOAc in PE) to give F-5.6 (200 mg, 65% yield over two steps). 1H NMR (400 MHz, CDCl₃) δ_{H} 5.30-5.27 (m, 1H), 2.66-2.56 (m, 1H), 2.55-2.45 (m, 1H), 2.42-2.34 (m, 2H), 2.15-2.01 (m, 2H), 1.98-1.93 (m, 1H), 1.77-1.59 (m, 8H), 1.50-1.15 (m, 11H), 1.09 (d, J=6.8 Hz, 6H), 1.06-1.02 (m, 1H), 0.99 (s, 3H), 0.97-0.91 (m, 2H), 0.89-0.83 (m, 7H). LC-ELSD/MS 30-90AB_2 min_E, purity>99%, MS ESI calcd. for C28H₄₅₀ [M−H₂O+H]⁺ 397.4, found 397.4.

### Synthesis of F-10 and F-5.7

To a solution of F-5.6 (180 mg, 0.434 mmol) in MeOH (5 mL) was added NaBH₄ (82.0 mg, 2.17 mmol) at 0° C. under N₂ and the resulting mixture was stirred at 25° C. for 2 h. The reaction mixture was quenched with 10% aqueous NH₄Cl (20 mL) added dropwise at 0° C. under N₂. The aqueous phase was extracted with EtOAc (2×20 mL). The combined organic layers were washed with brine (20 mL), dried over anhydrous Na₂SO₄, filtered, and concentrated under reduced pressure to give a residue. The residue was separated by SFC (DAICEL CHIRALPAK AD (250 mm*30 mm, 10 um); Mobile phase: A: CO₂ B: 0.1% NH₃H₂O EtOH; Gradient: from 30% to 30% of B; flow rate: 80 mL/min) to give F-10 (40.6 mg, 23% yield) and F-5.7 (43.2 mg, 23% yield).

F-10: ¹H NMR (400 MHz, CDCl₃) δ_{H} 5.33-5.24 (m, 1H), 3.40-3.28 (m, 1H), 2.36 (dd, J=2.0, 12.8 Hz, 1H), 2.15-1.91 (m, 3H), 1.80-1.55 (m, 11H), 1.47-1.01 (m, 13H), 0.99 (s, 3H), 0.98-0.88 (m, 12H), 0.85 (t, J=7.6 Hz, 3H). LC-ELSD/MS 30-90AB_2 min_E, purity>99%, MS ESI calcd. for C28H₄₇₀ [M−H₂O+H]⁺ 399.4, found 399.4.

F-5.7: ¹H NMR (400 MHz, CDCl₃) δ_{H} 5.34-5.23 (m, 1H), 3.42-3.24 (m, 1H), 2.37 (dd, J=2.4, 13.2 Hz, 1H), 2.15-1.92 (m, 3H), 1.80-1.58 (m, 8H), 1.51-1.34 (m, 8H), 1.32-1.01 (m, 8H), 0.99 (s, 3H), 0.98-0.88 (m, 12H), 0.87-0.83 (m, 3H). LC-ELSD/MS 30-90AB_2 min_E, purity>99%, MS ESI calcd. for C28H₄₇₀ [M−H₂O+H]⁺ 399.4, found 399.4.

### Example 74: Synthesis of (3S,5S,8R,9R,10S,13R,14S,17R)-3-ethyl-13-methyl-17-((2R,5S)-6,6,6-trifluoro-5-hydroxy-5-methylhexan-2-yl)hexadecahydro-1H-cyclopenta[a]phenanthren-3-ol (G-1) & (3S,5S,8R,9R,10S,13R,14S,17R)-3-ethyl-13-methyl-17-((2R,5R)-6,6,6-trifluoro-5-hydroxy-5-methylhexan-2-yl)hexadecahydro-1H-cyclopenta[a]phenanthren-3-ol (G-2)

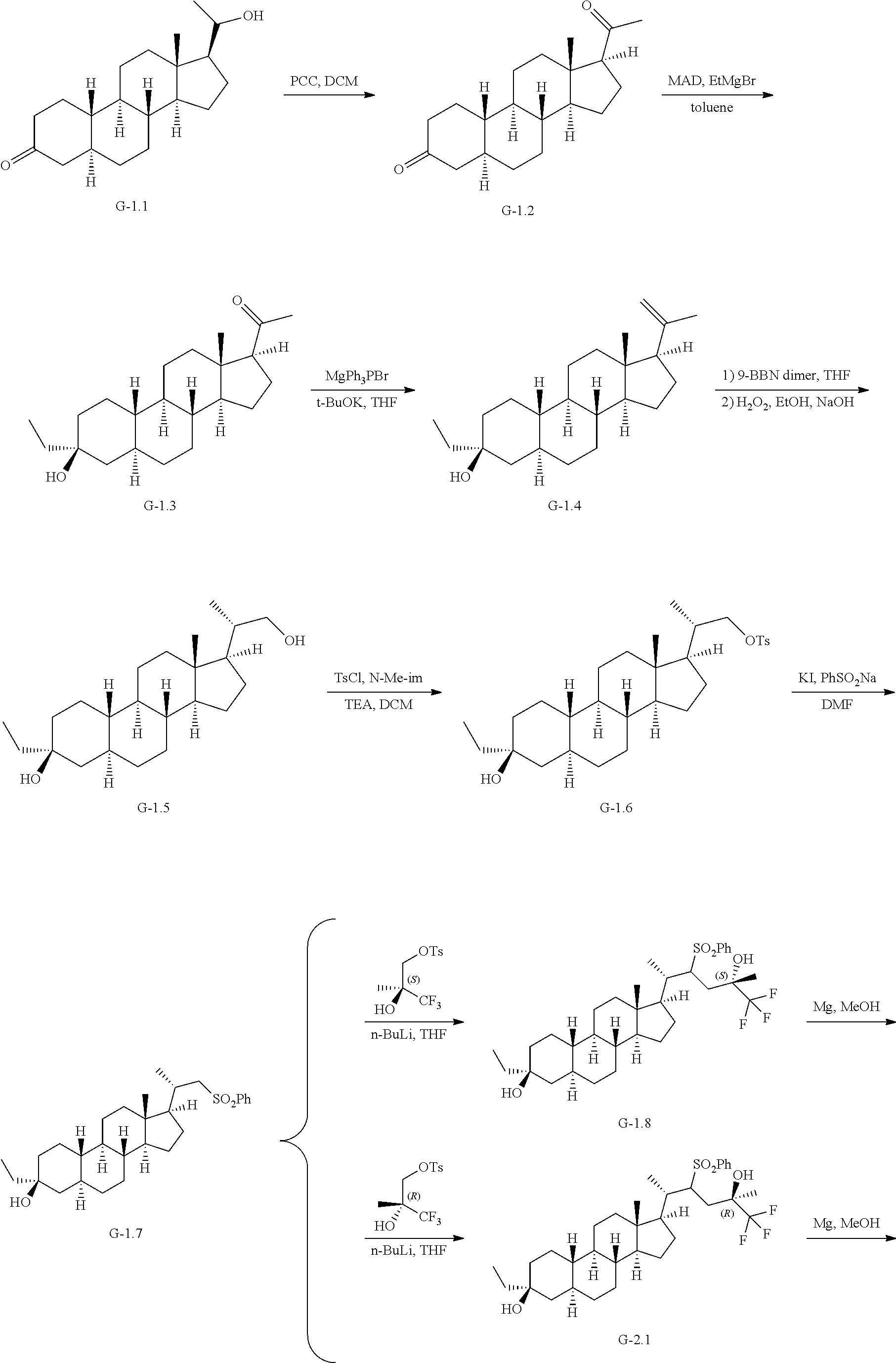

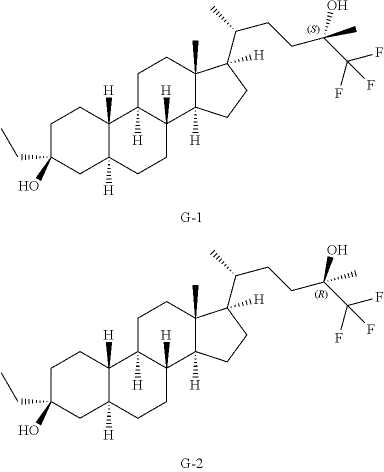

### Synthesis of G-1.2

To a solution of G-1.1 (7.80 g, 25.6 mmol) and silica gel (8.00 g) in DCM (100 mL) was added PCC (8.27 g, 38.4 mmol) and the reaction mixture was stirred at 25° C. for 12 hours. The solution was filtered and the filter cake was washed with DCM (100 mL). The combined filtrate was concentrated and the product was purified by silica gel column chromatography (PE/EtOAc=20/1) to give G-1.2 (3.80 g, 49.0%). ¹H NMR (400 MHz, CDCl₃) δ_{H} δ 2.56-2.52 (m, 1H), 2.43-2.36 (m, 1H), 2.24-2.35 (m, 3H), 2.21-2.09 (m, 5H), 2.07-2.03 (m, 1H), 1.90-1.79 (m, 1H), 1.75-1.61 (m, 4H), 1.50-1.38 (m, 2H), 1.28-1.09 (m, 7H), 1.03-0.91 (m, 1H), 0.81-0.71 (m, 1H), 0.65 (s, 3H). LCMS LC-ELSD/MS 30-90AB_2 min_E, purity 100%; MS ESI calcd. for C20H₃₀₀₂ [M+H]⁺ 303, found 303.

### Synthesis of G-1.3

To a solution of BHT (83.2 g, 377 mmol) in toluene (300 mL) under N₂ at 0° C. was added trimethylaluminum (2 M in toluene, 94.0 mL, 188 mmol) dropwise. The mixture was stirred at 25° C. for 1 hour. To the mixture was added a solution of G-1.2 (20.0 g, 66.1 mmol) in DCM (100 mL) drop-wise at −70° C. After stirring at -70° C. for 1 hour under N₂, EtMgBr (66.0 mL, 198 mmol, 3M in ethyl ether) was added dropwise at −70° C. The resulting solution was stirred at −70° C. for another 2 hours. The reaction mixture was poured into saturated citric acid (500 mL) at below 10° C. The aqueous phase was extracted with DCM (2×300 mL) and the combined organic phase was washed with brine (200 mL), dried over anhydrous Na₂SO₄, filtered, and concentrated. The residue was purified by flash column chromatography (0˜40% of EtOAc in PE) to give G-1.3 (21.0 g). ¹H NMR (400 MHz, CDCl₃) δ_{H} 2.57-2.48 (m, 1H), 2.21-2.13 (m, 1H), 2.11 (s, 3H), 2.03-1.96 (m, 1H), 1.88-1.74 (m, 3H), 1.70-1.50 (m, 7H), 1.46-1.16 (m, 6H), 1.15-0.77 (m, 9H), 0.75-0.64 (m, 2H), 0.61 (s, 3H).

### Synthesis of G-1.4

To a suspension of Ph₃PMeBr (45.0 g, 126 mmol) in THF (200 mL) was added t-BuOK (14.1 g, 126 mmol) at 20° C. under N₂. After stirring at 20° C. for 30 minutes, a solution of G-1.3 (21.0 g, 63.1 mmol) in THF (100 mL) was added drop-wise. After stirring for an additional 2 hours under N₂, the reaction mixture was poured into NH₄Cl (200 mL) and extracted with EtOAc (3×100 mL). The combined organic phase was washed with brine (200 mL), dried over anhydrous Na₂SO₄, filtered, and concentrated. The residue was purified by flash column chromatography (0˜20% of EtOAc in PE) to give G-1.4 (14.0 g, 67%). 1H NMR (400 MHz, CDCl₃) δ_{H} 4.84 (s, 1H), 4.70 (s, 1H), 2.04-1.98 (m, 1H), 1.88-1.76 (m, 4H), 1.75 (s, 3H), 1.74-1.62 (m, 5H), 1.61-1.52 (m, 4H), 1.36-0.99 (m, 9H), 0.97-0.82 (m, 5H), 0.76-0.61 (m, 2H), 0.57 (s, 3H).

### Synthesis of G-1.5

To a solution of G-1.4 (7.00 g, 21.1 mmol) in anhydrous THF (70 mL) was added 9-BBN dimer (10.2 g, 42.2 mmol) at 25° C. under N₂. The reaction mixture was stirred at 25° C. for 18 hours. To the resulting mixture was added ethanol (14.8 mL, 253 mmol) at 25° C., followed by NaOH aqueous (50.6 mL, 5.0 M, 253 mmol) and then H₂O₂ (25.3 mL, 10 M, 253 mmol) dropwise at 0° C. The reaction mixture was stirred at 80° C. for 3 hours and the mixture was cooled. The mixture was poured into Na₂S₂O₃ (100 mL, sat.) and the aqueous phase was extracted with EtOAc (2×100 mL). The combined organic phase was washed with brine (100 mL), dried over anhydrous Na₂SO₄, filtered, and concentrated. The residue was purified by flash column chromatography (25˜30% EtOAc in PE) to give G-1.5 (4.50 g, 61%). 1H NMR (400 MHz, CDCl₃) δ_{H} 3.67-3.60 (m, 1H), 3.41-3.32 (m, 1H), 1.98-1.60 (m, 12H), 1.52-1.09 (m, 11H), 1.08-0.85 (m, 12H), 0.68 (s, 3H).

### Synthesis of G-1.6

To a solution of G-1.5 (4.50 g, 12.9 mmol) in DCM (65 mL) was added N-methyl-imidazole (1.58 g, 19.3 mmol), TEA (5.32 mL, 38.6 mmol), and TsCl (4.91 g, 25.8 mmol). The mixture was stirred at 25° C. for 4 hours and then poured into NaHCO₃ (100 mL, saturated). The aqueous phase was extracted with DCM (2×100 mL). The combined organic phase was washed with brine (100 mL), dried over anhydrous Na₂SO₄, filtered, and concentrated. Purification by flash column chromatography (15˜25% EtOAc in PE) provided G-1.6 (6.10 g, 94.1%). ¹H NMR (400 MHz, CDCl₃) δ_{H} 7.80-7.74 (m, 2H), 7.36-7.31 (m, 2H), 4.01-3.91 (m, 1H), 3.81-3.76 (m, 1H), 2.45 (s, 3H), 1.84-1.73 (m, 7H), 1.69-1.62 (m, 3H), 1.35-1.25 (m, 4H), 1.20-1.08 (m, 5H), 1.06-0.95 (m, 8H), 0.90-0.85 (m, 7H), 0.62 (s, 3H).

### Synthesis of G-1.7

To a solution of G-1.6 (6.10 g, 12.1 mmol) in DMF (60 mL) and THF (30 mL) was added potassium iodide (9.62 g, 58.0 mmol) at 25° C. The mixture was stirred at 50° C. for 4 hours and then sodium benzenesulfinate (5.95 g, 36.3 mmol) was added. The mixture was stirred at 40° C. for 16 hours then poured into water (200 mL) and filtered. The filter cake was washed with water (200 mL). The resulting filter cake was dissolved in DCM (200 mL), washed with brine (200 mL), dried over Na₂SO₄, filtered, and concentrated. Purification by flash column chromatography (15˜25% EtOAc in PE) provided G-1.7 (3.00 g, 52%). 1H NMR (400 MHz, CDCl₃) δ_{H} 7.93-7.89 (m, 2H), 7.67-7.62 (m, 1H), 7.59-7.54 (m, 2H), 3.18-3.10 (m, 1H), 2.89-2.79 (m, 1H), 2.14-2.05 (m, 1H), 1.93-1.60 (m, 7H), 1.55-1.50 (m, 3H), 1.33-1.14 (m, 7H), 1.12-0.97 (m, 10H), 0.92-0.78 (m, 6H), 0.63 (s, 3H).

### Synthesis of G-1.8

To a solution of G-1.7 (500 mg, 1.05 mmol) in THF (4 mL) at -70° C. under N₂ was added dropwise n-BuLi (1.68 mL, 4.2 mmol, 2.5M in hexane). The reaction mixture was stirred at -70° C. for 0.5 hour. DIPA (424 mg, 4.2 mmol) was added dropwise at −70° C. for 0.5 hour. (S)-3,3,3-trifluoro-2-hydroxy-2-methylpropyl 4-methylbenzenesulfonate (405 mg, 1.36 mmol) was added dropwise at -70° C. The reaction mixture was warmed to 25° C. slowly and stirred at 25° C. for 18 hours. The reaction mixture was quenched with saturated NH₄Cl (10 mL) and extracted with EtOAc (2×10 mL). The combined organic phase was washed with 10% NH₄Cl (10 mL), dried over Na₂SO₄, filtered, and concentrated to give G-1.8 (600 mg), which was carried directly to the next step.

### Synthesis of G-1

To a solution of G-1.8 (600 mg, 1.01 mmol) in 50 mL of dry methanol was added nickel (II) chloride (26.0 mg, 0.2020 mmol) and Mg powder (1.45 g, 60.6 mmol) in four portions under N₂ at 65° C. The reaction mixture was quenched with citric acid (50 mL, 10% aqueous), which was added dropwise until the solid was dissolved. The mixture was extracted with EtOAc (3×50 mL) and the combined organic layer was washed with sat. NaHCO₃ (50 mL) and brine (50 mL), dried over Na₂SO₄, filtered, and concentrated. The residue was purified by flash column chromatography (0˜15% of EtOAc in PE). Further purification by recrystallization from CH₃CN (10 mL) followed by lyophilization provided G-1 (150 mg, 32.3%). ¹H NMR (400 MHz, CDCl₃) δ_{H} 1.98-1.57 (m, 11H), 1.54-1.25 (m, 9H), 1.24-0.96 (m, 12H), 0.96-0.71 (m, 9H), 0.66 (s, 3H), 0.64-0.56 (m, 1H). ¹⁹F NMR (376 MHz, CDCl₃) δ_{F} −83.15 (s). LC-ELSD/MS 30-90AB_2 min_E, purity>99%; MS ESI calcd. for C₂₇H₄₅F₃O₂ [M−H₂O+H]⁺ 441.4, found 441.4.

### Synthesis of G-2.1

To a solution of G-1.7 (500 mg, 1.05 mmol) in THF (3 mL) under N₂ at −70° C. was added dropwise n-BuLi (1.68 mL, 4.2 mmol, 2.5M in hexane). The reaction mixture was stirred at -70° C. for 0.5 hour. DIPA (424 mg, 4.2 mmol) was added dropwise at -70° C. for 0.5 hour. (R)-3,3,3-trifluoro-2-hydroxy-2-methylpropyl 4-methylbenzenesulfonate (626 mg, 2.10 mmol) was added dropwise at −70° C. The reaction mixture was warmed to 25° C. slowly and stirred at 25° C. for 18 hours. The reaction mixture was quenched with saturated NH₄Cl (10 mL) and extracted with EtOAc (2×10 mL). The combined organic phase was washed with 10% NH₄Cl (10 mL), dried over Na₂SO₄, filtered, and concentrated to give G-2.1 (600 mg) which was carried directly to the next step.

### Synthesis of G-2

To a solution of G-2.1 (1.00 g, 1.66 mmol) in 50 mL of dry methanol (50 mL) was added nickel (II) chloride (42.8 mg, 0.332 mmol) and Mg powder (2.39 g, 99.6 mmol) in four portions under N₂ at 65° C. The reaction mixture was quenched with citric acid (50 mL, 10% aqueous), which was added dropwise until the solid was dissolved. The mixture was extracted with EtOAc (3×50 mL) and the combined organic layer was washed with brine (50 mL), dried over Na₂SO₄, filtered, and concentrated. The residue was purified by flash column chromatography (0˜15% of EtOAc in PE). Further purification by recrystallization from CH₃CN (10 mL) followed by lyophilization provided G-2 (72.5 mg, 35%). ¹H NMR (400 MHz, CDCl₃) δ_{H} 1.97-1.57 (m, 10H), 1.54-1.24 (m, 11H), 1.21-0.96 (m, 11H), 0.95-0.70 (m, 9H), 0.66 (s, 3H), 0.63-0.54 (m, 1H). 1° F. NMR (376 MHz, CDCl₃) δ_{F} −82.83 (s). LC-ELSD/MS 30-90AB_2 min_E, purity 100%; MS ESI calcd. for C₂₇H₄₅F₃O₂ [M−H₂O+H]⁺ 441.4, found 441.4.

### Example 75: Synthesis of (3S,5S,8S,9S,10S,13R,14S,17R)-3,10-diethyl-17-((R)-6-hydroxy-6-methylheptan-2-yl)-13-methylhexadecahydro-1H-cyclopenta[a]phenanthren-3-ol (G-3)

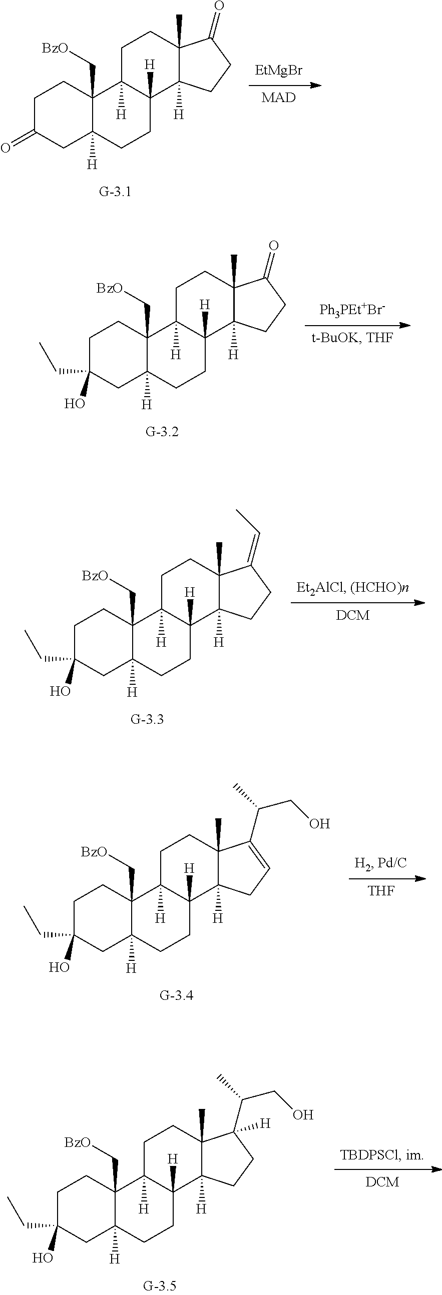

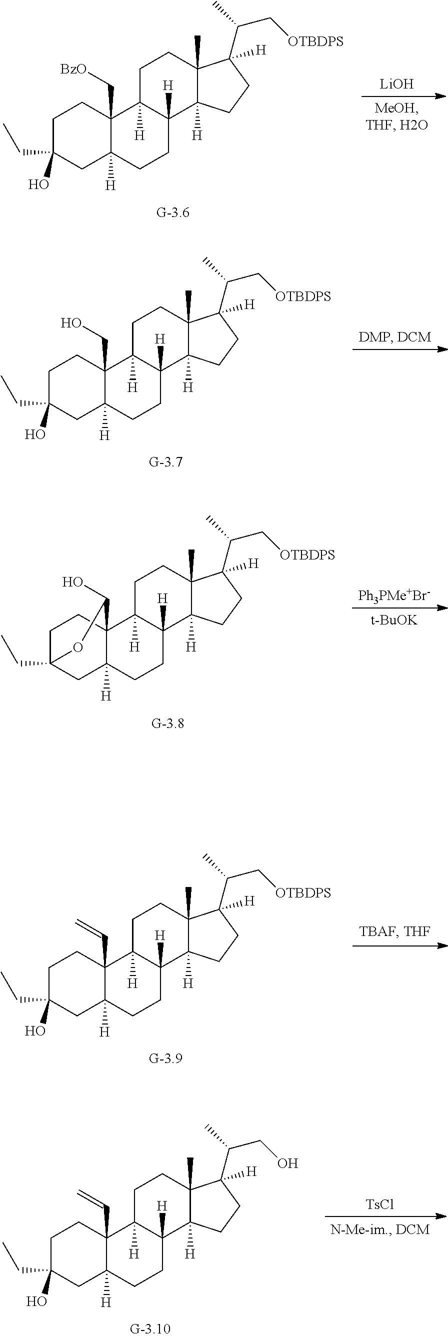

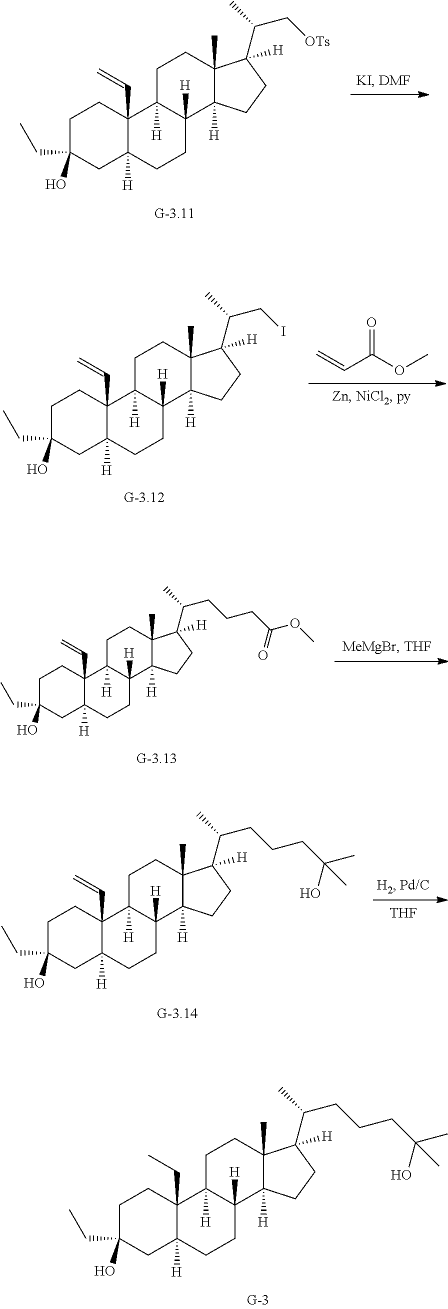

### Synthesis of G-3.2

To a solution of BHT (170 g, 771 mol) in toluene (500 mL) was added AlMe₃ (192 mL, 385 mmol, 2 M in toluene) at 0° C. After stirring at 15° C. for 0.5 hour, a solution of G-3.1 (50.0 g, 122 mmol) in DCM (300 mL) was added at −70° C. under N₂. The mixture was stirred at -70° C. for 1 hour and EtMgBr (366 mmol, 122 mL, 3.0 M in diethyl ether) was added dropwise at −70° C. After stirring at -70° C. for 1 hour, the reaction was combined with a second reaction using 100 g of G-3.1 and conducted using the same procedure. The combined reaction mixture was quenched by saturated aqueous citric acid (1500 mL) and extracted with ethyl acetate (2×1000 mL). The combined organic layer was washed with brine (500 mL), dried over Na₂SO₄, filtered, and concentrated. Purification by silica gel chromatography (PE/EtOAc=0 to 3/1) provided G-3.2 (100 g). ¹H NMR (400 MHz, CDCl₃) δ_{H} 8.06-7.96 (m, 2H), 7.63-7.53 (m, 1H), 7.51-7.43 (m, 2H), 4.72-4.45 (m, 2H), 2.11-1.84 (m, 4H), 1.83-1.59 (m, 7H), 1.58-1.44 (m, 5H), 1.43-1.31 (m, 4H), 1.31-1.10 (m, 4H), 0.94-0.84 (m, 4H), 0.81 (s, 3H).

### Synthesis of G-3.3

To a mixture of EtPPh₃Br (248 g, 669 mmol) in THF (1000 mL) was added t-BuOK (75.0 g, 669 mmol) at 20° C. under N₂. After stirring at 60° C. for 30 minutes, G-3.2 (98.0 g, 223 mmol) in THF (200 mL) was added in portions below 60° C. and the mixture was stirred for 16 hours. The mixture was quenched with 10% NH₄Cl aqueous (1000 mL), extracted with EtOAc (2×1000 mL). The combined organic phase was concentrated and purified by silica gel chromatography (0˜20% EtOAc in PE) to give G-3.3 (15.0 g, 37.5%). ¹H NMR (400 MHz, CDCl₃) δ_{H} 8.06-7.99 (m, 2H), 7.61-7.54 (m, 1H), 7.49-7.45 (m, 2H), 5.14-5.01 (m, 1H), 4.64-4.47 (m, 2H), 2.44-1.69 (m, 10H), 1.57-1.45 (m, 8H), 1.37-1.28 (m, 7H), 1.22-1.10 (m, 5H), 0.82 (s, 3H), 0.81-0.77 (m, 1H).

### Synthesis of G-3.4

To a mixture of (CH₂O) n (9.96 g, 332 mmol) in DCM (1.5 L) under N₂ was added Et₂AlCl (532 mL, 532 mmol, 1M in hexanes) dropwise at −78° C. over 15 minutes. A solution of G-3.3 (60.0 g, 133 mmol) in DCM (500 mL) was added at −78° C. After stirring at 25° C. for 12 hours, the mixture was slowly poured into aqueous 10% NH₄Cl (1000 mL). The organic layer was separated and washed with aqueous 10% NH₄Cl (800 mL), dried over Na₂SO₄, filtered, and concentrated to give G-3.4 (60.0 g). ¹H NMR (400 MHz, CDCl₃) δ_{H} 8.11-7.93 (m, 2H), 7.61-7.54 (m, 1H), 7.49-7.45 (m, 2H), 5.48-5.36 (m, 1H), 4.69-4.45 (m, 2H), 3.68-3.45 (m, 2H), 2.44-1.70 (m, 9H), 1.68-1.45 (m, 8H), 1.46-1.18 (m, 6H), 1.06-0.98 (m, 4H), 0.94-0.82 (m, 5H), 0.73 (s, 3H).

### Synthesis of G-3.5

To a solution of G-3.4 (60.0 g, 124 mmol) in THF (1000 mL) was added Pd/C (10.0 g) under N₂. The suspension was degassed under vacuum and purged with H₂ three times. After stirring under H₂ (15 psi) at 20° C. for 16 hours, the mixture was filtered through a pad of Celite and washed with THF (2×200 mL). The filtrate was concentrated, triturated with MeCN (500 mL) and purified by combi-flash (0˜15% of EtOAc in PE) to give G-3.5 (40.0 g, 66.8%). ¹H NMR (400 MHz, CDCl₃) δ_{H} 8.08-7.98 (m, 2H), 7.64-7.37 (m, 3H), 4.62-4.45 (m, 2H), 3.68-3.56 (m, 1H), 3.40-3.30 (m, 1H), 2.41-1.66 (m, 8H), 1.56-1.39 (m, 7H), 1.36-1.12 (m, 8H), 1.05-0.73 (m, 11H), 0.63 (s, 3H).

### Synthesis of G-3.6

To a solution of G-3.5 (40.0 g, 82.8 mmol) in DCM (800 mL) was added imidazole (11.2 g, 165 mmol) and TBDPSCI (25.0 g, 91.0 mmol). After stirring at 15° C. for 16 hours, the reaction mixture was washed with water (1000 mL), extracted with DCM (2×500 mL), dried over Na₂SO₄, filtered, and concentrated to give G-3.6 (60.0 g). ¹H NMR (400 MHz, CDCl₃) δ_{H} 8.08-7.98 (m, 2H), 7.79-7.63 (m, 6H), 7.59-7.55 (m, 1H), 7.50-7.33 (m, 11H), 4.64-4.45 (m, 2H), 3.65-3.53 (m, 1H), 3.39-3.26 (m, 1H), 2.46-1.77 (m, 5H), 1.53-1.21 (m, 10H), 1.06-0.01 (m, 13H), 1.01-0.77 (m, 9H), 0.58 (s, 3H).

### Synthesis of G-3.7

To a solution of G-3.6 (58.0 g, 80.4 mmol) in MeOH/THF/H₂O (4:2:1, 420 mL) was added LiOH. H₂O (33.7 g, 804 mmol) at 20° C. After stirring for 5 hours at 20° C., the mixture was poured into water (1000 mL) and extracted with EtOAc (3×10 mL). The combined organics were dried over anhydrous Na₂SO₄, filtered, concentrated, and purified by silica gel chromatography (0˜40% of EtOAc in PE) to give G-3.7 (30.0 g, 60.4%). ¹H NMR (400 MHz, CDCl₃) δ_{H} 7.76-7.62 (m, 4H), 7.45-7.32 (m, 6H), 3.97-3.80 (m, 2H), 3.68-3.54 (m, 1H), 3.41-3.27 (m, 1H), 2.23-2.10 (m, 1H), 2.04-1.90 (m, 2H), 1.81-1.35 (m, 14H), 1.33-1.15 (m, 6H), 1.12-1.03 (m, 13H), 1.02-0.94 (m, 2H), 0.93-0.85 (m, 4H), 0.78-0.62 (m, 4H).

### Synthesis of G-3.8

To a solution of G-3.7 (20.0 g, 32.3 mmol) in DCM (200 mL) at 25° C. was added DMP (20.5 g, 48.4 mmol). After stirring at 25° C. for 1 hour, saturated NaHCO₃ (500 mL) was added dropwise, saturated aqueous Na₂S₂O₃ (500 mL) was added and the mixture was stirred at 25° C. for another 10 minutes. The resulting mixture was extracted with DCM (400 mL) and filtered. The filtrate was concentrated to give G-3.8 (20.0 g), which was used directly in the next step.

### Synthesis of G-3.9

To a solution of MePh3PBr (28.2 g, 79.0 mmol) in THF (150 mL) was added t-BuOK (8.90 g, 79.0 mmol) at 25° C. The mixture was stirred at 50° C. for 1 hour. A solution of G-3.8 (20.0 g, 31.6 mmol) in THF (30 mL) was added to the reaction mixture below 50° C. The reaction mixture was stirred at 50° C. for 16 hours then cooled and poured into saturated aqueous NH₄Cl (500 mL). The aqueous layer was extracted with EtOAc (3×100 mL). The combined organic layer was washed with brine (200 mL), dried over anhydrous Na₂SO₄, filtered, and concentrated. Purification by flash column chromatography (0˜15% of EtOAc in PE) provided G-3.9 (11.1 g).

### Synthesis of G-3.10

To a solution of G-3.9 (11.1 g, 18.1 mmol) in THF (120 mL) was added TBAF (36.2 mL, 36.2 mmol, 1 M) and the mixture was stirred at 20° C. for 16 hours. The mixture was poured into H₂O (500 mL) and the aqueous layer was extracted with EtOAc (3×100 mL). The combined organic layer was washed with brine (200 mL), dried over anhydrous Na₂SO₄, filtered, and concentrated to give G-3.10 (12.3 g).

### Synthesis of G-3.11

To a solution of G-3.10 (12.3 g, 32.8 mmol) in DCM (120 mL) was added 1-methyl-1H-imidazole (12.8 mL, 163 mmol) and TsCl (12.5 g, 65.6 mmol) and the mixture was stirred at 20° C. for 16 hours. The mixture was poured into H₂O (500 mL) and the aqueous layer was extracted with EtOAc (3×150 mL). The combined organic layer was washed with brine (200 mL), dried over anhydrous Na₂SO₄, filtered, and concentrated. Purification by flash column chromatography (0˜25% of EtOAc in PE) and further separation by SFC (Column: DAICEL CHIRALPAK AD (250 mm*50 mm, 10 um), Condition: 0.1% NH₃H₂O ETOH, Begin B: 45%, End B: 45%, FlowRate (ml/min): 220, Injections: 120) provided G-3.11 (2.30 g).

### Synthesis of G-3.12

To a solution of G-3.11 (2.30 g, 4.34 mmol) in DMF (16 mL) and THF (8 mL) was added KI (3.45 g, 20.8 mmol) at 25° C. After stirring at 60° C. for 5 hours, the mixture was poured into water (20 mL) and stirred for 20 minutes. The aqueous phase was extracted with EtOAc (3×20 mL). The combined organic phase was washed with brine (2×20 mL), dried over anhydrous Na₂SO₄, filtered, and concentrated to give G-3.12 (2.10 g). ¹H NMR (400 MHz, CDCl₃) δ_{H} 5.97-5.74 (m, 1H), 5.37-5.35 (m, 1H), 5.05-4.95 (m, 1H), 3.36-3.27 (m, 1H), 3.18-3.11 (m, 1H), 2.12-2.05 (m, 1H), 1.92-1.76 (m, 2H), 1.75-1.58 (m, 4H), 1.55-1.52 (m, 2H), 1.52-1.34 (m, 3H), 1.34-1.18 (m, 6H), 1.17-1.09 (m, 5H), 1.08-1.01 (m, 1H), 1.01-0.98 (m, 3H), 0.97-0.92 (m, 2H), 0.90-0.85 (m, 3H), 0.77-0.68 (m, 1H), 0.60 (s, 3H).

### Synthesis of G-3.13

To a mixture of Zn powder (188 mg, 2.88 mmol) in dry pyridine (2 mL) was slowly added methyl acrylate (247 mg, 2.88 mmol) and NiCl₂ (80.0 mg, 0.618 mmol) at 25° C. under N₂. After stirring for 2 hours, a solution of G-3.12 (200 mg, 0.412 mmol) was added to the mixture slowly. After stirring at 25° C. for another 2 hours, to the mixture was added saturated NH₄Cl (10 mL). The mixture was filtered through a pad of silica gel and the filtrate was extracted with EtOAc (2×20 mL). The organic phase was dried over Na₂SO₄, filtered, and concentrated. The residue was purified by flash column chromatography (0˜20% of EtOAc in PE) and lyophilized to give G-3.13 (12.7 mg, 100%). ¹H NMR (400 MHz, CDCl₃) δ_{H} 5.93-5.84 (m, 1H), 5.35 (d, J=11.6 Hz, 1H), 5.00 (d, J=18.0 Hz, 1H), 3.66 (s, 3H), 2.34-2.18 (m, 2H), 2.12-2.05 (m, 1H), 1.94-1.86 (m, 1H), 1.75-1.57 (m, 5H), 1.52-1.41 (m, 3H), 1.41-1.30 (m, 5H), 1.30-1.24 (m, 5H), 1.22-1.16 (m, 2H), 1.12-1.00 (m, 5H), 0.99-0.85 (m, 9H), 0.76-0.68 (m, 1H), 0.55 (s, 3H). LC-ELSD/MS 30-90AB_2 min_E, purity>99%, MS ESI calcd. for C₂₉H₄₈₀₃ [M−H₂O+H]⁺ 427.4, found 427.4.

### Synthesis of G-3.14

A solution of MeMgBr (5.0 mL, 15.0 mmol 3M) in THF (13 mL) was reacted with G-3.13 (337 mg, 0.757 mmol) in THF (2 mL) at 0° C. under N₂. After stirring at 0° C. for 10 minutes, the mixture was poured into saturated aqueous NH₄Cl (20 mL), stirred for 20 minutes, and extracted with EtOAc (3×40 mL). The combined organic phase was dried over anhydrous Na₂SO₄, filtered, and concentrated. The residue was lyophilized to give G-3.14 (7.00 mg, 100%). ¹H NMR (400 MHz, CDCl₃) δ_{H} 5.98-5.79 (m, 1H), 5.35 (d, J=11.6 Hz, 1H), 5.01 (d, J=17.6 Hz, 1H), 2.15-2.05 (m, 1H), 1.98-1.85 (m, 1H), 1.77-1.67 (m, 1H), 1.77-1.58 (m, 2H), 1.52-1.48 (m, 2H), 1.45-1.32 (m, 8H), 1.30-1.24 (m, 6H), 1.22-1.16 (m, 10H), 1.14-0.97 (m, 6H), 0.96-0.93 (m, 1H), 0.92-0.86 (m, 7H), 0.77-0.71 (m, 1H), 0.56 (s, 3H). LC-ELSD/MS 30-90AB_2 min_E, purity>99%, MS ESI calcd. for C₃₀H₅₂₀₂ [M−2H₂O+H]⁺ 409.4, found 409.4.

### Synthesis of G-3

To a mixture of G-3.14 (50.0 mg, 0.112 mmol) in THF (10 mL)) was added Pd/C (wet, 10%, 200 mg) under N₂. The suspension was degassed under vacuum and purged with H₂ three times. The mixture was stirred under H₂ (40 psi) at 40° C. for 14 hours. The mixture was filtered, washed with THF (2×30 mL), and concentrated. The residue was purified by flash column chromatography (0˜25% of EtO Ac in PE) and further purified by lyophilization to give G-3 (27.6 mg). ¹H NMR (400 MHz, CDCl₃) δ_{H} 2.00-1.91 (m, 2H), 1.86-1.74 (m, 1H), 1.69-1.58 (m, 4H), 1.50-1.31 (m, 12H), 1.30-1.17 (m, 12H), 1.16-0.99 (m, 7H), 0.97-0.86 (m, 10H), 0.84-0.72 (m, 2H), 0.68-0.61 (m, 4H). LC-ELSD/MS 30-90AB_2 min_E, purity>99%, MS ESI calcd. for C₃₀H₅₄O₂ [M−2H₂O+H]⁺ 411.4, found 411.4.

### Example 76: Synthesis of (3S,5S,8S,9S,10S,13R,14S,17R)-3,10-diethyl-13-methyl-17-((2R,5R)-6,6,6-trifluoro-5-hydroxy-5-methylhexan-2-yl)hexadecahydro-1H-cyclopenta[a]phenanthren-3-ol (G-4) & (3S,5S,8S,9S,10S,13R,14S,17R)-3,10-diethyl-13-methyl-17-((2R,5S)-6,6,6-trifluoro-5-hydroxy-5-methylhexan-2-yl)hexadecahydro-1H-cyclopenta[a]phenanthren-3-ol (G-5)

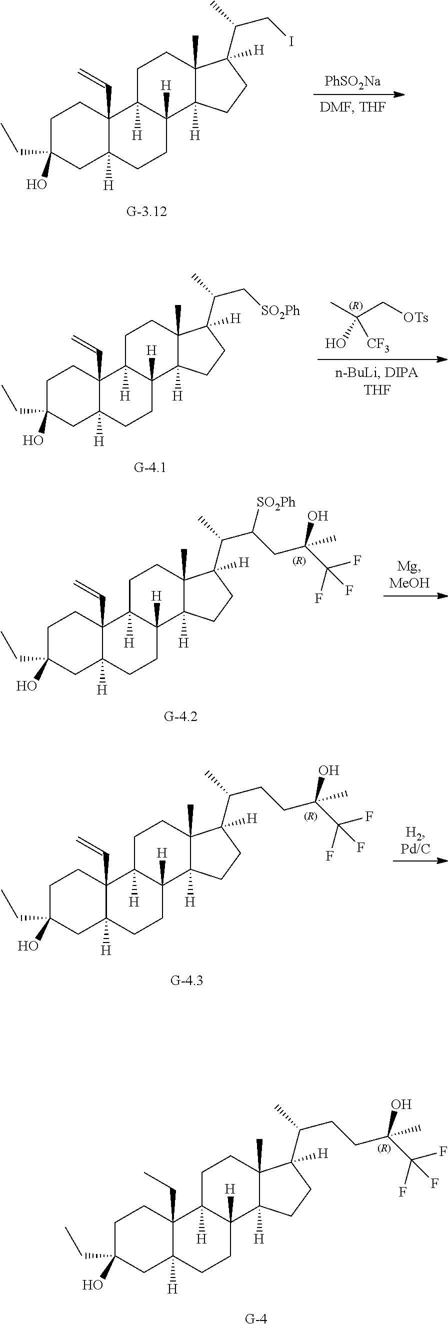

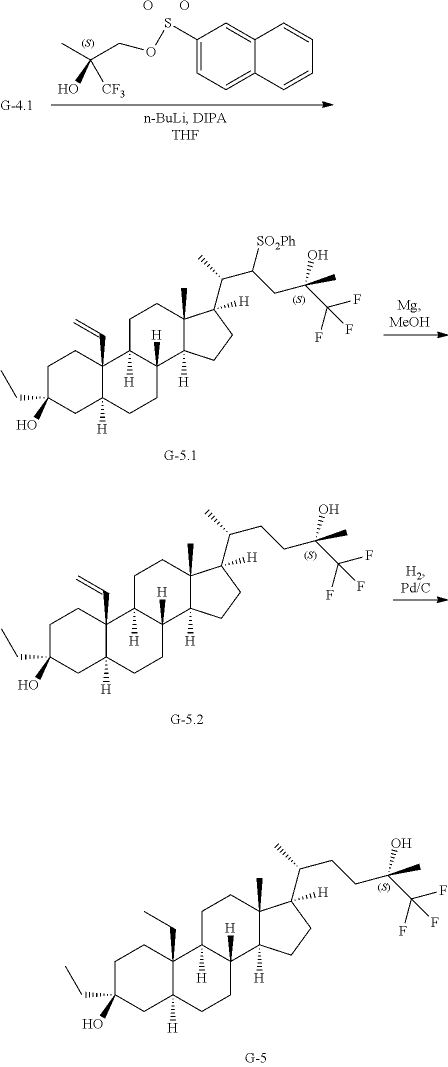

### Synthesis of G-4.1

To the solution of G-3.12 (500 mg, 1.03 mmol) in DMF (5 mL) was added THF (1 mL) and PhSO₂Na (169 mg, 1.03 mmol) at 25° C. After stirring at 60° C. for 4 hours, the reaction mixture was poured into water (20 mL). The mixture was filtered and the filter cake was washed with water (20 mL). The resulting filter cake was dissolved in DCM (20 mL), washed with brine (20 mL), dried over Na₂SO₄, filtered, and concentrated. The residue was purified by flash column chromatography (0˜20% EtOAc in PE) to give G-4.1 (400 mg). 1H NMR (400 MHz, CDCl₃) δ_{H} 7.96-7.85 (m, 2H), 7.69-7.61 (m, 1H), 7.60-7.50 (m, 2H), 5.91-5.79 (m, 1H), 5.39-5.32 (m, 1H), 5.06-4.95 (m, 1H), 3.12 (d, J=7.2 Hz, 1H), 2.88-2.78 (m, 1H), 2.12-2.03 (m, 2H), 1.92-1.84 (m, 1H), 1.74-1.63 (m, 3H), 1.55-1.46 (m, 4H), 1.42-1.33 (m, 2H), 1.32-1.23 (m, 5H), 1.19-1.10 (m, 6H), 1.09-0.96 (m, 3H), 0.95-0.84 (m, 6H), 0.74-0.66 (m, 1H), 0.53 (s, 3H)

### Synthesis of G-4.2

To a solution of G-4.1 (400 mg, 0.801 mmol) in THF (5 mL) was added dropwise n-BuLi (1.28 mL, 3.20 mmol, 2.5M in hexane) under N₂ at -70° C. The reaction mixture was stirred at −70° C. for 0.5 hour. DIPA (323 mg, 3.20 mmol) was added dropwise at −70° C. over 0.5 hour. (R)-3,3,3-trifluoro-2-hydroxy-2-methylpropyl 4-methylbenzenesulfonate (238 mg, 0.801 mmol) was added dropwise at −70° C. The reaction mixture was warmed to 25° C. slowly and stirred at 25° C. for 18 hours. The reaction mixture was quenched with saturated aqueous NH₄Cl (10 mL) and extracted with EtOAc (2×10 mL). The combined organic phase was washed with 10% aqueous NH₄Cl (10 mL), dried over Na₂SO₄, filtered, and concentrated to give G-4.2 (420 mg), which was used directly in the next step.

### Synthesis of G-4.3

To a solution of G-4.2 (420 mg, 0.672 mmol) in 50 mL of dry methanol was added nickel (II) chloride (17.2 mg, 0.134 mmol) and Mg powder (967 mg, 40.3 mmol) in four portions under N₂ at 65° C. The reaction mixture was quenched with citric acid (50 mL, 10%) which was added dropwise until solid was dissolved. After extraction with EtOAc (3×50 mL), the organic layer was washed with sat. NaHCO₃ (50 mL), brine (50 mL), dried over Na₂SO₄, filtered, and concentrated. The residue was purified by flash column chromatography (0˜15% of EtOAc in PE) twice to give G-4.3 (57.0 mg, 71%). ¹H NMR (400 MHz, CDCl₃) δ_{H} 5.94-5.83 (m, 1H), 5.35 (d, J=11.2 Hz, 1H), 5.03 (dd, J=18.0 Hz, 1H), 2.14-2.04 (m, 1H), 1.95-1.87 (m, 1H), 1.82-1.67 (m, 3H), 1.66-1.58 (m, 3H), 1.53-1.35 (m, 7H), 1.35-1.17 (m, 12H), 1.14-1.00 (m, 4H), 0.97-0.82 (m, 9H), 0.79-0.69 (m, 1H), 0.62-0.52 (m, 3H). ¹⁹F NMR (376 MHz, CDCl₃) δ_{F} −82.82 (s)

### Synthesis of G-4

To a mixture of G-4.3 (51.0 mg, 0.105 mmol) in THF (10 mL)) was added Pd/C (wet, 20%, 200 mg) under N₂. The suspension was degassed under vacuum and purged with H₂ three times. The mixture was stirred under H₂ (40 psi) at 40° C. for 24 hours. The mixture was filtered, washed with THF (2×30 mL), and concentrated. Purification by flash column chromatography (0˜15% EtOAc in PE) and lyophilization provided G-4 (16.2 mg, 32%). 1H NMR (400 MHz, CDCl₃) δ_{H} 1.99-1.88 (m, 2H), 1.88-1.70 (m, 3H), 1.69-1.57 (m, 5H), 1.51-1.39 (m, 7H), 1.37-1.30 (m, 5H), 1.29-1.17 (m, 5H), 1.16-0.98 (m, 6H), 0.98-0.86 (m, 10H), 0.86-0.70 (m, 2H), 0.70-0.61 (m, 4H). ¹⁹F NMR (376 MHz, CDCl₃) δ_{F} −82.82 (s). LC-ELSD/MS 30-90AB_2 min_E, purity>99%, MS ESI calcd. for C₂₉H₄₉F₃O₂ [M−H₂O+H]⁺ 469.4, found 469.4.

### Synthesis of G-5.1

To a solution of G-4.1 (250 mg, 0.501 mmol) in THF (2 mL) was added dropwise n-BuLi (0.80 mL, 2.0 mmol, 2.5 M in hexane) under N₂ at −70° C. The reaction mixture was stirred at -70° C. for 0.5 hour. DIPA (0.279 mL, 2.00 mmol) was added dropwise at −70° C. over 0.5 hour. (S)-3,3,3-trifluoro-2-hydroxy-2-methylpropyl naphthalene-2-sulfonate (167 mg, 0.500 mmol) in THF (1 mL) was added dropwise at −70° C. The reaction mixture was warmed to 25° C. slowly, and stirred at 25° C. for 18 hours. The reaction mixture was quenched with saturated aqueous NH₄Cl (10 mL) and extracted with EtOAc (2×10 mL). The combined organic phase was washed with 10% aqueous NH₄Cl (10 mL), dried over Na₂SO₄, filtered, and concentrated to give G-5.1 (320 mg) which was used directly in the next step.

### Synthesis of G-5.2

To a solution of G-5.1 (320 mg, 0.512 mmol) in dry MeOH (50 mL) was added NiCl₂ (13.1 mg, 0.102 mmol) and Mg powder (736 mg, 30.7 mmol) in four portions under N₂ at 65° C. The reaction mixture was quenched with citric acid (50 mL, 10% aqueous), which was added dropwise until the solid was dissolved. After extraction with EtOAc (3×50 mL), the combined organic layer was washed with sat. aqueous NaHCO₃ (50 mL) and brine (50 mL), dried over Na₂SO₄, filtered, and concentrated. The residue was purified by flash column chromatography (0˜20% of EtOAc in PE) to give G-5.2 (120 mg). ¹H NMR (400 MHz, CDCl₃) δ_{H} 5.96-5.74 (m, 1H), 5.35 (d, J=11.2 Hz, 1H), 5.06-4.88 (m, 1H), 2.13-2.05 (m, 1H), 1.95-1.87 (m, 1H), 1.85-1.60 (m, 6H), 1.55-1.46 (m, 4H), 1.45-1.35 (m, 3H), 1.34-1.05 (m, 15H), 1.04-0.84 (m, 10H), 0.77-0.69 (m, 1H), 0.56 (s, 3H). ¹⁹F NMR (376 MHz, CDCl₃) δ_{F} −83.13 (s).

### Synthesis of G-5

To a mixture of G-5.2 (100 mg, 0.206 mmol) in THF (10 mL) was added Pd/C (wet, 10%, 200 mg) under N₂. The suspension was degassed under vacuum and purged with H₂ three times. After stirring under H₂ (40 psi) at 40° C. for 24 hours, the mixture was filtered, washed with THF (2×30 mL), and concentrated. Purification by flash column chromatography (0˜15% of EtOAc in PE) and trituration with MeOH (5 mL) and H₂O (5 mL) twice gave the product. Further purification by flash column chromatography (0˜20% of EtOAc in PE) and lyophilization provided G-5 (12.3 mg, 37%). ¹H NMR (400 MHz, CDCl₃) δ_{H} 2.00-1.90 (m, 2H), 1.87-1.72 (m, 2H), 1.71-1.59 (m, 5H), 1.49-1.38 (m, 6H), 1.37-1.22 (m, 9H), 1.21-1.01 (m, 8H), 0.99-0.85 (m, 11H), 0.85-0.73 (m, 2H), 0.69-0.62 (m, 4H). ¹⁹F NMR (376 MHz, CDCl₃) δ_{F} −83.13 (s). LC-ELSD/MS 30-90AB_2 min_E, purity>99%, MS ESI calcd. for C₂₉H₄₉F₃O₂ [M−H₂O+H]⁺ 469.4, found 469.4.

### Example 77: Synthesis of (2S,4aR,4bS,6aR,7R,10aS,10bS)-2,4a,6a-trimethyl-7-((2R,5S)-6,6,6-trifluoro-5-hydroxy-5-methylhexan-2-yl)-1,2,3,4,4a,4b,5,6,6a,7,8,9,10,10a,10b,11-hexadecahydrochrysen-2-ol (H-1)

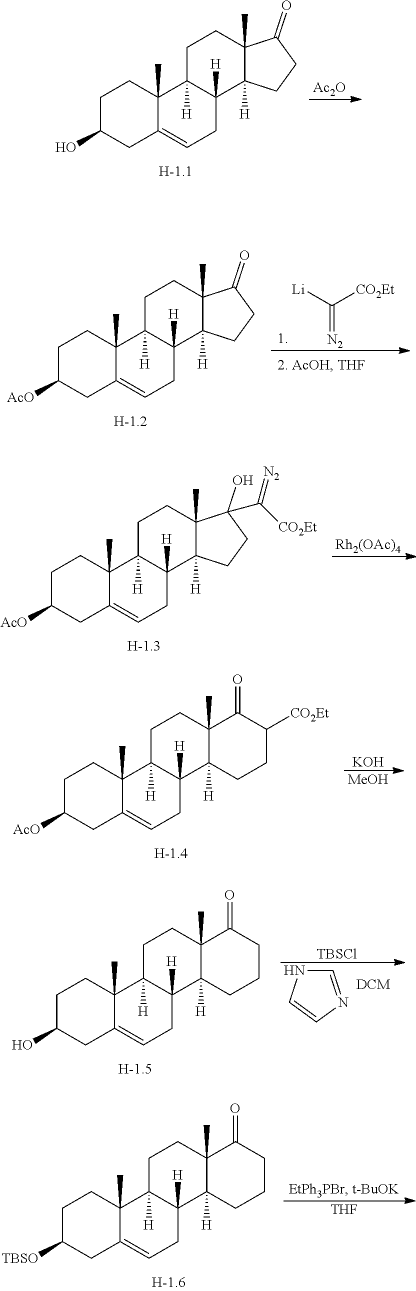

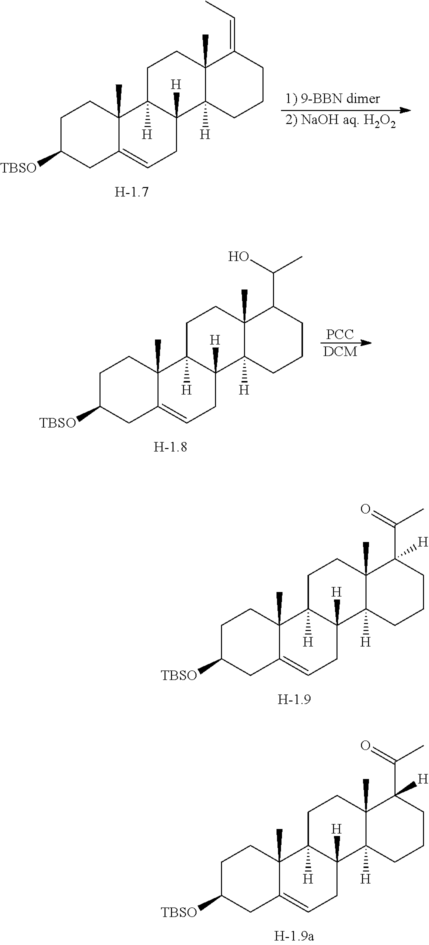

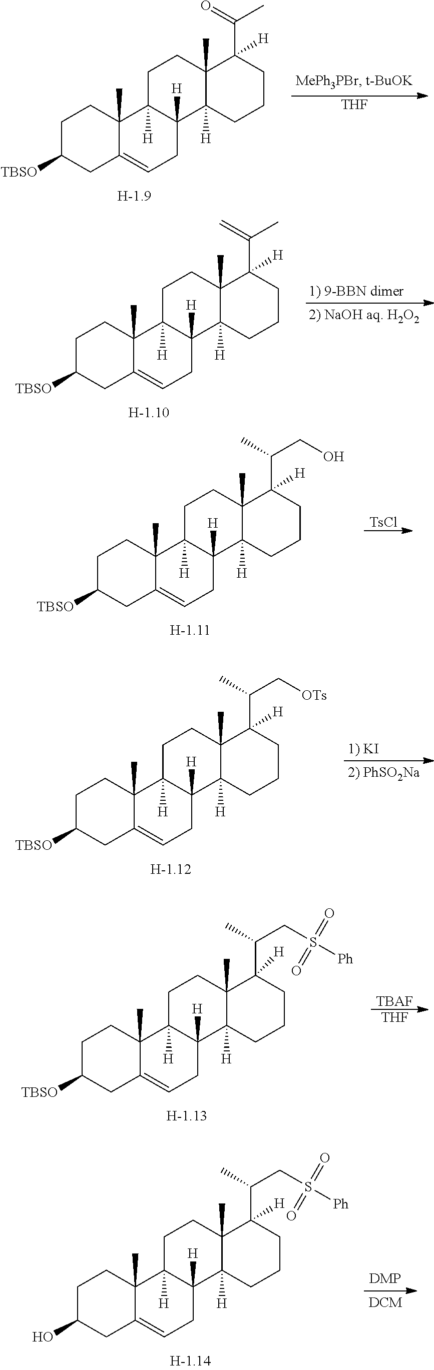

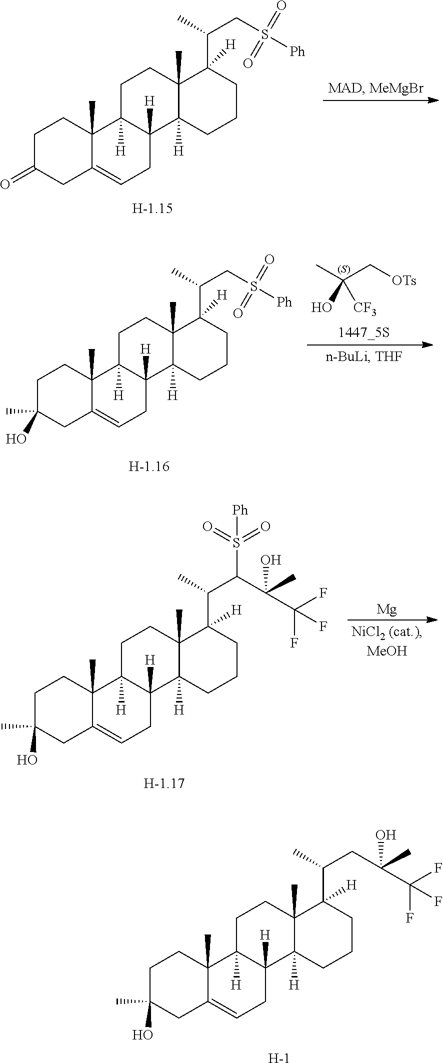

### Synthesis of H-1.2

To a solution of H-1.1 (100 g, 346 mmol) in acetic anhydride (560 mL) and pyridine (240 mL) was added DMAP (2.11 g, 17.3 mmol) at 25° C. and the mixture was stirred for 0.5 hour. The reaction mixture was poured into water (1 L). The mixture was filtered, and the filter cake was washed with water (2×500 mL). The filter cake was dissolved in EtOAc (500 mL) and the organic phase was washed with HCl aq. (1 M, 500 mL), 5% NaHCO₃ aq. (500 mL) and brine (200 mL), dried over Na₂SO₄, filtered, and concentrated to give H-1.2 (112 g, 98%). ¹H NMR (400 MHz, CDCl₃) δ_{H} 5.45-5.35 (m, 1H), 4.69-4.50 (m, 1H), 2.52-2.25 (m, 3H), 2.20-1.75 (m, 9H), 1.75-1.45 (m, 6H), 1.38-0.98 (m, 7H), 0.89 (s, 3H).

### Synthesis of H-1.3

A cold (0° C.) solution of lithium diisopropylamide prepared from addition of n-butyl-lithium in hexane (193 mL, 483 mmol, 2.5 M in THF) to diisopropylamine (76.2 mL, 543 mmol) in THF (50 mL) was added to a stirred solution of H-1.2 (50.0 g, 151 mmol) and ethyl diazoacetate (51.5 g, 452 mmol) in THF (500 mL) at -78° C. over 30 minutes. The mixture was stirred at −78° C. for 45 minutes. Then acetic acid (31.0 mL, 543 mmol) in THF (50 mL) was added over 20 minutes, and the mixture was allowed to warm to 20° C. Water (100 mL) was added. The organic layer was separated, and the aqueous solution was extracted with diethyl ether (3×100 mL). The combined organic phase was washed with brine (2×50 mL), dried over Na₂SO₄, filtered, and concentrated to give H-1.3 (65.0 g). 1H NMR (400 MHz, CDCl₃) δ_{H} 5.45-5.35 (m, 1H), 4.82-4.52 (m, 2H), 4.35-4.25 (m, 3H), 2.40-2.15 (m, 3H), 2.15-1.80 (m, 8H), 1.80-1.22 (m, 8H), 1.22-0.90 (m, 11H).

### Synthesis of H-1.4

To a solution of H-1.3 (65.0 g, 146 mmol) in DME (200 mL) was added rhodium (II) acetate dimer (322 mg, 0.73 mol) in one portion at 25° C. The mixture was stirred at 25° C. for 30 minutes. The mixture was concentrated to give H-1.4 (75.0 g), which was used directly in the next step.

### Synthesis of H-1.5

To a solution of H-1.4 (75.0 g, 180 mmol) in MeOH (500 mL) was added KOH (50.4 g, 899 mmol) in one portion at 25° C. The mixture was stirred at 65° C. for 16 hours. Water (1000 mL) was added and the mixture was extracted with EtOAc (3×500 mL). The combined organic phase was washed with brine (500 mL), dried over Na₂SO₄, filtered, and concentrated. The residue was purified by flash column chromatography (0˜30% EtOAc in PE) to give H-1.5 (54.0 g, 99%). ¹H NMR (400 MHz, CDCl₃) δ_{H} 5.45-5.35 (m, 1H), 3.60-3.45 (m, 1H), 2.75-2.60 (m, 1H), 2.39-2.00 (m, 5H), 1.95-1.75 (m, 4H), 1.75-1.31 (m, 8H), 1.31-0.90 (m, 10H).

### Synthesis of H-1.6

To a solution of H-1.5 (54.0 g, 178 mmol) in DCM (500 mL) was added imidazole (24.2 g, 356 mmol) and TBSCI (40.2 g, 267 mmol) at 25° C. The reaction mixture was stirred for 16 hours and then water was added (2 L). The organic layer was separated and the aqueous phase was extracted with DCM (2×500 mL). The combined organic phase was washed with brine (2×200 mL), dried over anhydrous Na₂SO₄, filtered, and concentrated to give H-1.6 (73.3 g). ¹H NMR (400 MHz, CDCl₃) δ_{H} 5.45-5.35 (m, 1H), 3.60-3.45 (m, 1H), 2.75-2.60 (m, 1H), 2.39-2.00 (m, 5H), 1.95-1.35 (m, 16H), 1.35-0.80 (m, 14H), 0.05 (s, 6H).

### Synthesis of H-1.7

To a suspension of bromo(ethyl)triphenylphosphorane (196 g, 528 mmol) in THF (800 mL) was added 1-BuOK (59.2 g, 528 mmol) at 15° C. The mixture was stirred at 45° C. for 30 minutes. H-1.6 (73.3 g, 176 mmol) in THF (200 mL) was added dropwise at 45° C. and the mixture was stirred for 16 hours. The mixture was quenched with NH₄Cl (200 mL, sat. aq.) and the organic layer was separated. The aqueous phase was extracted with EtOAc (3×500 mL), the combined organic phase was dried over Na₂SO₄, filtered, and concentrated. The residue was triturated with MeOH/H₂O (500 mL/500 mL) at 25° C. to give H-1.7 (51.0 g). 1H NMR (400 MHz, CDCl₃) δ_{H} 5.45-5.35 (m, 1H), 5.25-5.10 (m, 1H), 3.60-3.45 (m, 1H), 2.38-2.10 (m, 5H), 2.10-1.80 (m, 3H), 1.80-1.30 (m, 14H), 1.30-0.75 (m, 17H), 0.05 (s, 6H).

### Synthesis of H-1.8

To a solution of H-1.7 (51.0 g, 19.5 mmol) in THF (200 mL) was added 9-BBN dimer (57.5 g, 236 mmol) at 0° C. under N₂. The solution was stirred at 65° C. for 2 hours. After cooling to 0° C., a solution of NaOH (235 mL, 5M, 1.18 mol) was added very slowly. After addition, H₂O₂ (133 g, 1.18 mol, 30% in water) was added slowly and the inner temperature was maintained below 10° C. The mixture was stirred at 75° C. under N₂ for 1 hour. The mixture was re-cooled to 25° C. and then water (2000 ml) was added. The precipitate was collected by filtration and washed with H₂O (2×500 mL) to give H-1.8 (60.0 g). ¹H NMR (400 MHz, CDCl₃) δ_{H} 5.45-5.35 (m, 1H), 3.80-3.70 (m, 1H), 3.55-3.45 (m, 1H), 2.38-2.00 (m, 5H), 2.00-1.35 (m, 21H), 1.35-0.70 (m, 15H), 0.05 (s, 6H).

### Synthesis of H-1.9 & H-1.9A

To a suspension of H-1.8 (60.0 g, 134 mmol) in DCM (500 mL) was added silica gel (58.0 g) and PCC (57.6 g, 268 mmol) at 25° C. and the mixture was stirred for 2 hours. The mixture was filtered, and the filter cake was washed with DCM (2×300 mL). The combined filtrate was concentrated in vacuum and purified by flash column chromatography (0˜20% EtOAc in PE) to give a mixture of H-1.9A (13.0 g) and H-1.9 (16.0 g, 26%). ¹H NMR: 1.9 (400 MHz, CDCl₃) δ_{H} 5.45-5.35 (m, 1H), 3.55-3.40 (m, 1H), 2.35-2.05 (m, 7H), 1.89-1.68 (m, 4H), 1.68-1.15 (m, 9H), 1.15-0.75 (m, 20H), 0.05 (s, 6H).

### Synthesis of H-1.10

To a suspension of MePPh₃Br (25.6 g, 71.8 mmol) in THF (200 mL) was added t-BuOK (8.05 g, 71.8 mmol) at 25° C. After addition, the reaction mixture was heated to 45° C. and stirred for 1 hour. Then H-1.9 (16.0 g, 35.9 mmol) was added and the reaction mixture was stirred at 45° C. for 16 hours. The mixture was treated with NH₄Cl (100 mL, sat. aq.) and the organic layer was separated. The aqueous phase was extracted with EtOAc (2×100 mL). The combined organic phase was washed with saturated brine (2×200 mL), dried over anhydrous Na₂SO₄, filtered, and concentrated. The residue was triturated with MeOH/H₂O (100 mL/100 mL) at 20° C. to give H-1.10 (13.0 g, 82%). ¹H NMR (400 MHz, CDCl₃) δ_{H} 5.33-5.27 (m, 1H), 4.80 (s, 1H), 4.65-4.58 (m, 1H), 3.55-3.40 (m, 1H), 2.30-2.05 (m, 3H), 1.89-1.20 (m, 17H), 1.20-0.75 (m, 20H), 0.06 (s, 6H).

### Synthesis of H-1.11

To a solution of H-1.10 (13.0 g, 29.3 mmol) in THF (100 mL) was added 9-BBN dimer (14.3 g, 58.6 mmol) at 0° C. under N₂. After stirring at 0° C. for 2 hours, EtOH (16.9 mL, 293 mmol) was added. Then a solution of NaOH (58.6 mL, 5M, 293 mmol) was added very slowly. After addition, H₂O₂ (33.2 g, 293 mmol, 30% in water) was added slowly and the inner temperature was maintained below 10° C. The mixture was stirred at 75° C. under N₂ for 1 hour and re-cooled to 25° C. The mixture was added to water (2000 ml) and stirred 30 minutes. The precipitate was collected by filtration and washed with H₂O (2×500 mL) to give H-1.11 (11.0 g, 81%). ¹H NMR (400 MHz, CDCl₃) δ_{H} 5.35-5.25 (m, 1H), 3.80-3.70 (m, 1H), 3.55-3.40 (m, 2H), 3.35-3.25 (m, 1H), 2.34-2.05 (m, 3H), 2.00-1.85 (m, 2H, 1.85-1.10 (m, 10H), 1.10-0.95 (m, 11H), 0.95-0.70 (m, 15H), 0.05 (s, 6H).

### Synthesis of H-1.12

To a solution of H-1.11 (5.00 g, 10.8 mmol) in DCM (50 mL) was added 1-methyl-1H-imidazole (1.33 g, 16.2 mmol) and TEA (2.18 g, 21.6 mmol) at 25° C. TsCl (4.11 g, 21.6 mmol) was added and the reaction mixture was stirred at 25° C. for 2 hours. The mixture was washed with water (50 mL), H₂O/pyridine (50 mL/5 mL), and brine (20 mL), dried over Na₂SO₄, filtered, and concentrated to give H-1.12 (7.00 g), which was used directly in the next step.

### Synthesis of H-1.13

To a solution of H-1.12 (7.00 g, 11.3 mmol) in DMF (200 mL) was added KI (9.37 g, 56.5 mmol) at 25° C. The mixture was stirred at 55° C. for 72 hours. To the mixture was added PhSO₂Na·2H₂O (20.0 g, 122 mmol). The mixture was stirred at 60° C. for 72 hours. The reaction mixture was cooled to 25° C., poured into water (500 mL), and filtered. The filter cake was washed with water (3×50 mL). The resulting filter cake was dissolved in DCM (100 mL), washed with water (2×30 mL) and brine (2×20 mL), dried over Na₂SO₄, filtered, and concentrated. The residue was purified by flash column chromatography (0˜10% EtOAc in PE) to give H-1.13 (1.50 g, 21%). ¹H NMR (400 MHz, CDCl₃) δ_{H} 7.95-7.85 (m, 2H), 7.75-7.60 (m, 1H), 7.60-7.55 (m, 2H), 5.35-5.25 (m, 1H), 3.55-3.45 (m, 1H), 3.25-3.15 (m, 1H), 2.75-2.65 (m, 1H), 2.65-2.50 (m, 1H), 2.30-2.00 (m, 4H), 1.85-1.70 (m, 4H), 1.70-1.10 (m, 7H), 1.10-0.62 (m, 25H), 0.05 (m, 6H).

### Synthesis of H-1.14

To a solution of H-1.13 (1.50 g, 2.56 mmol) in THF (20 mL) was added TBAF (2.00 g, 7.68 mmol) at 25° C. The mixture was stirred at 75° C. for 5 hours, treated with water (30 mL) and extracted with EtOAc (2×30 mL). The combined organic phase was washed with water (20 mL) and brine (100 mL), dried over anhydrous Na₂SO₄, filtered, and concentrated. The residue was purified by flash column chromatography (0˜20% EtOAc in PE) to give H-1.14 (1.10 g, 92%). ¹H NMR (400 MHz, CDCl₃) δ_{H} 7.89-7.82 (m, 2H), 7.75-7.60 (m, 1H), 7.60-7.55 (m, 2H), 5.35-5.25 (m, 1H), 3.60-3.40 (m, 1H), 2.78-2.65 (m, 1H), 2.60-2.45 (m, 1H), 2.35-2.00 (m, 3H), 2.40-1.70 (m, 4H), 1.65-1.39 (m, 6H), 1.39-1.10 (m, 7H), 1.10-0.78 (m, 9H), 0.78-0.61 (m, 4H).

### Synthesis of H-1.15

To a solution of H-1.14 (500 mg, 1.06 mmol) in DCM (30 mL) was added DMP (1.79 g, 4.24 mmol), followed by adding H₂O (3.81 mg, 212 umol). The reaction was stirred at 25° C. for 30 minutes and treated with aqueous saturated NaHCO₃ (20 mL) and saturated Na₂S₂O₃ (20 mL) solution. The mixture was extracted with DCM (2×30 mL). The combined organic layer was washed with aqueous saturated NaHCO₃ (2×10 mL) and brine (10 mL), dried over Na₂SO₄, filtered, and concentrated to give H-1.15 (450 mg, 91%). ¹H NMR (400 MHz, CDCl₃) δ_{H} 7.89-7.82 (m, 2H), 7.75-7.60 (m, 1H), 7.60-7.55 (m, 2H), 5.35-5.25 (m, 1H), 3.25-3.15 (m, 1H), 2.85-2.65 (m, 1H), 2.65-2.40 (m, 1H), 2.40-2.25 (m, 1H), 2.25-2.00 (m, 2H), 1.95-1.79 (m, 2H), 1.79-1.30 (m, 5H), 1.30-0.80 (m, 17H), 0.80-0.65 (m, 4H).

### Synthesis of H-1.16

To a solution of BHT (4.67 g, 21.2 mmol) in toluene (10 mL) under nitrogen at 0° C. was added trimethylaluminum (5.30 mL, 2 M, 10.6 mmol) dropwise. The mixture was stirred at 0° C. for 30 minutes and used directly as a solution of MAD without further purification. To the solution of MAD under nitrogen at −78° C. was added a solution of H-1.15 (450 mg, 0.96 mmol) in toluene (10 mL) drop wise. After stirring at −78° C. for 30 minutes, MeMgBr (3 M in diethyl ether, 3.19 mL, 9.60 mmol) was added drop wise. The resulting mixture was stirred at −78° C. for 3 hours. The reaction mixture was poured into ice-cooled aqueous citric acid (20 mL). The mixture was extracted with EtOAc (2×30 mL). The combined organic layer was washed with brine (30 mL), dried over anhydrous Na₂SO₄, filtered, and concentrated. The residue was purified by flash column chromatography (0˜30% EtOAc in PE) to give H-1.16 (400 mg, 86%). ¹H NMR (400 MHz, CDCl₃) δ_{H} 8.02-7.82 (m, 2H), 7.75-7.60 (m, 1H), 7.60-7.55 (m, 2H), 5.30-5.22 (m, 1H), 3.25-3.10 (m, 1H), 2.80-2.65 (m, 1H), 2.65-2.48 (m, 1H), 2.48-2.30 (m, 1H), 2.18-1.95 (m, 3H), 1.95-1.65 (m, 4H), 1.65-1.05 (m, 14H), 1.05-0.78 (m, 9H), 0.78-0.60 (m, 4H).

### Synthesis of H-1.17

To a solution of H-1.16 (200 mg, 0.412 mol) in THF (3 mL) under N₂ at −70° C. was added n-BuLi (0.575 mL, 1.44 mmol, 2.5 M in hexane). After stirring at −70° C. for 30 minutes, a solution of 1447_5S (147 mg, 0.494 mol) in THF (2 mL) was added. Then reaction was stirred at -70° C. for 30 minutes and then stirred at 25° C. for 16 hours. Then the reaction was quenched with sat. aq. NH₄Cl (10 mL) and extracted with EtOAc (3×10 mL). The combined organic layer was washed brine, dried over Na₂SO₄, filtered, and concentrated give H-1.17 (249 mg, 99%). ¹H NMR (400 MHz, CDCl₃) δ_{H} 8.02-7.82 (m, 2H), 7.80-7.55 (m, 3H), 5.30-5.22 (m, 1H), 3.70-3.60 (m, 1H), 2.55-2.48 (m, 1H), 2.48-2.30 (m, 2H), 2.15-1.95 (m, 10H), 1.95-1.00 (m, 9H), 1.00-0.75 (m, 15H), 0.75-0.49 (m, 5H).

### Synthesis of H-1

To a solution of H-1.17 (249 mg, 0.407 mmol) in 50 mL of dry methanol was added nickel (II) chloride (5 mg) and Mg powder (393 mg, 16.2 mmol) in four portions under N₂ at 60° C. The reaction mixture was quenched by 1M HCl (150 mL), which was added dropwise until solid was dissolved. The mixture was extracted with EtOAc (3×50 mL) and the organic layer was washed with sat. aq. NaHCO₃ (50 mL) and brine (50 mL), dried over Na₂SO₄, filtered, and concentrated. The residue was purified by flash column chromatography (0˜20% EtOAc in PE) to give H-1 (80.0 mg, 40%). ¹H NMR (400 MHz, CDCl₃) δ_{H} 5.35-5.25 (m, 1H), 2.49-2.33 (m, 1H), 2.20-2.08 (m, 1H), 2.08-1.88 (m, 2H), 1.88-1.68 (m, 6H), 1.68-1.30 (m, 11H), 1.30-1.05 (m, 6H), 1.05-0.85 (m, 12H), 0.85-0.68 (m, 5H). ¹⁹F NMR (376 MHz, CDCl₃) δ_{F} −83.08 (s) LC-ELSD/MS 30-90AB_2 min_E, purity >99%, MS ESI calcd. for C28H₄₅F₃O₂ [M+H−H₂O]⁺ 453.3, found 453.3.

### Example 78: Synthesis of (2S,4aS,4bS,6aR,7R,10aS,10bR,12aS)-2-ethyl-7-((2R,5R)-5-hydroxy-6-methylheptan-2-yl)-4a,6a-dimethyloctadecahydrochrysen-2-ol (H-2)

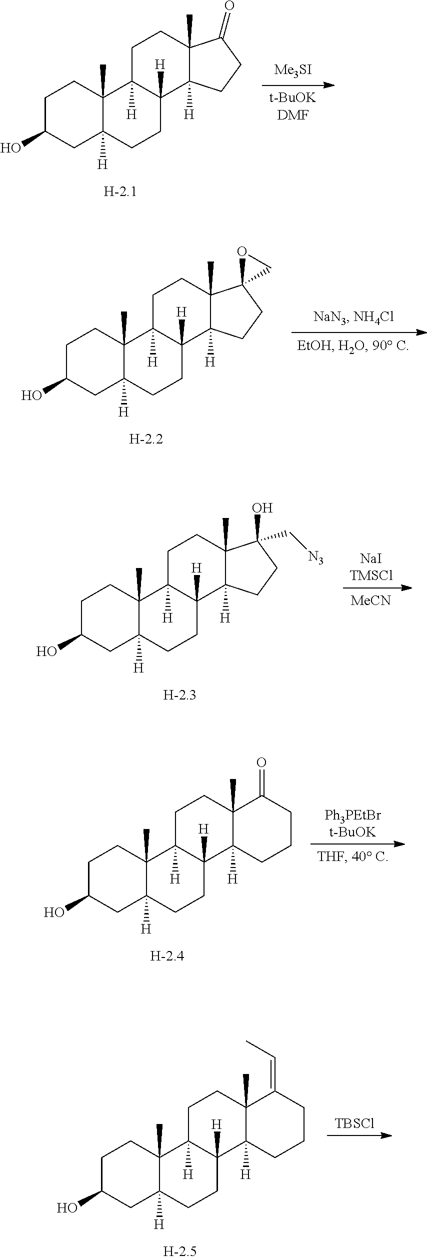

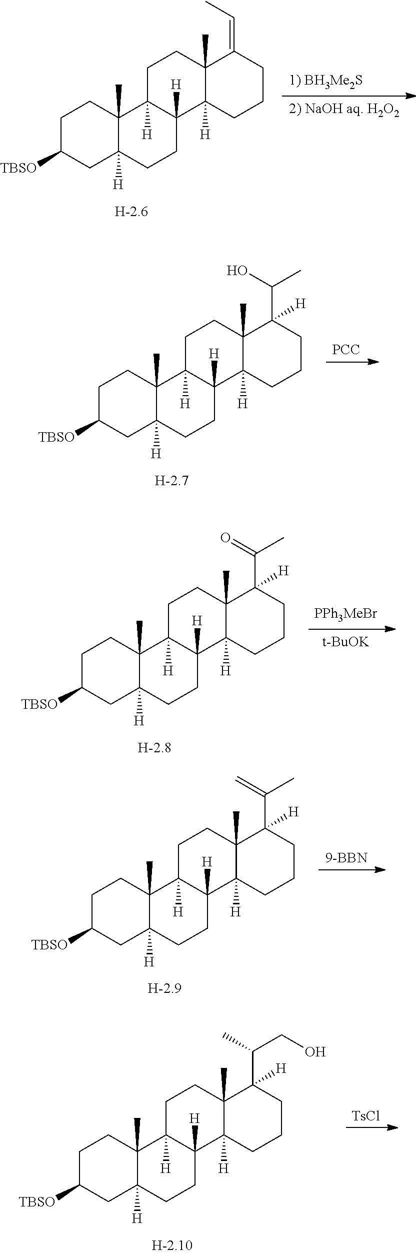

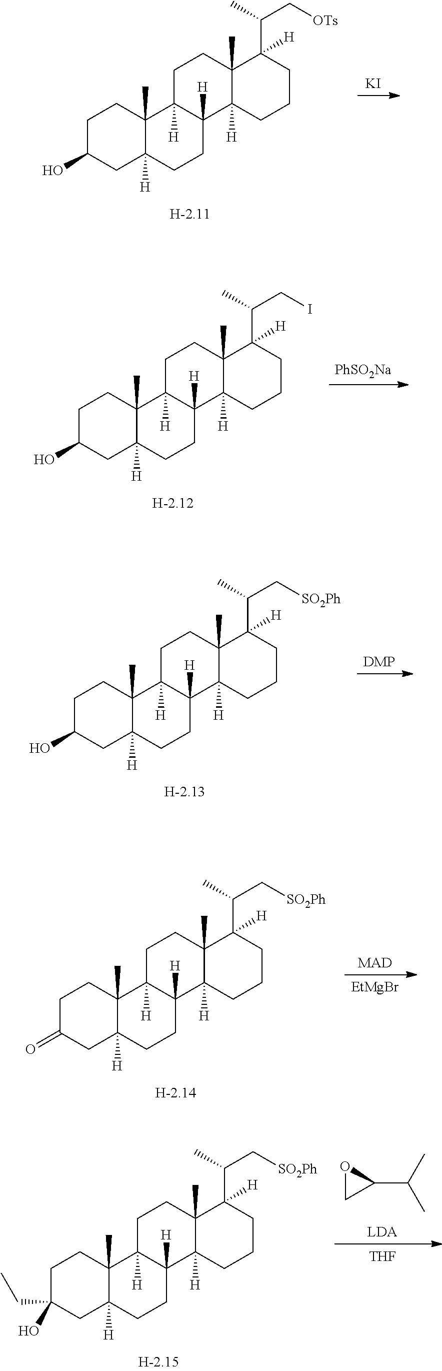

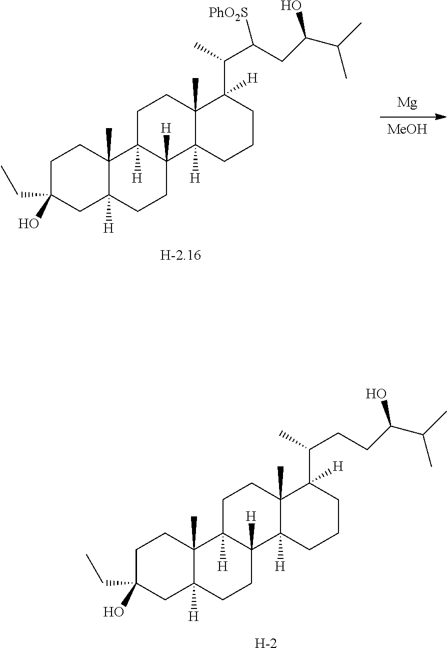

### Synthesis of H-2.2

Me₃SI (140 g, 688 mmol) and t-BuOK (77.0 g, 688 mmol) were added in one portion to a solution of H-2.1 (100 g, 344 mmol) in anhydrous DMF (1.5 L) at 5° C. The reaction mixture was heated to 25° C. and stirred for 16 hours. Brine (500 mL) was added and the mixture was extracted with EtOAc (3×500 mL). The combined organic layers were washed with H₂O (2×1000 mL), dried over Na₂SO₄, filtered, and concentrated. Trituration with PE (400 mL) provided H-2.2 (58.0 g, 43%). The mother liquor was concentrated and purified by combi flash (0˜30% EtOAc in PE) to give H-2.2 (37.0 g, 28%). ¹H NMR (400 MHz, CDCl₃) δ_{H} 3.65-3.51 (m, 1H), 2.85-2.80 (m, 1H), 2.59-2.52 (m, 1H), 2.01-1.91 (m, 1H), 1.84-1.73 (m, 5H), 1.52-0.89 (m, 16H), 0.87 (s, 3H), 0.82 (s, 3H), 0.72-0.65 (m, 1H).

### Synthesis of H-2.3

A solution of H-2.2 (32.0 g, 105 mmol), sodium azide (20.4 g, 315 mmol), and ammonium chloride (19.6 g, 367 mmol) in EtOH (1.5 L) and water (300 mL) was heated at 90° C. for 16 hours. The reaction mixture was combined with two additional batches prepared using the same procedure from a total amount of 63.0 g of H-2.2. The mixture was cooled to 15° C. and water (10 L) was added. The precipitate was filtered and washed with water (3×1 L). The resulting solid was dissolved in THF (500 mL), then dried over Na₂SO₄, filtered, and concentrated to give H-2.3 (95.0 g, 88%). ¹H NMR (400 MHz, CDCl₃) δ_{H} 3.55-3.50 (m, 2H), 3.26-3.22 (m, 1H), 2.03-1.58 (m, 8H), 1.54-0.94 (m, 14H), 0.88 (s, 3H), 0.82 (s, 3H), 0.70-0.65 (m, 2H).

### Synthesis of H-2.4

Sodium iodide (204 g, 1.36 mol) was added to a solution of H-2.3 (47.5 g, 136 mmol) in dry acetonitrile (1.5 L). Trimethylsilyl chloride (147 g, 1.36 mol) was added dropwise. The reaction mixture was stirred at 15° C. for 1 hour. The reaction mixture was combined with another batch prepared from 47.5 g of H-2.3. An aqueous solution of hydrochloric acid (5%, 1 L) was added and the suspension was extracted with EtOAc (2×1 L). The combined organic extracts were washed with saturated aqueous Na₂S₂O₃ (2×1 L) and brine (1 L), dried over Na₂SO₄, filtered, and concentrated to give H-2.4 (84.0 g). Purification by re-crystallization in MeCN (300 mL) at 20° C. provided H-2.4 (70.0 g, 84%). ¹H NMR (400 MHz, CDCl₃) δ_{H} 3.65-3.55 (m, 2H), 2.65-2.54 (m, 1H), 2.20-1.58 (m, 8H), 1.53-1.11 (m, 12H), 1.08 (s, 3H), 1.07-0.81 (m, 2H), 0.80 (s, 3H), 0.69-0.65 (m, 1H).

### Synthesis of H-2.5

To a mixture of bromo(ethyl)triphenylphosphorane (291 g, 786 mmol) in THF (800 mL) was added t-BuOK (88.1 g, 786 mmol) at 15° C. and the mixture was stirred at 50° C. for 30 minutes. H-2.4 (40.0 g, 131 mmol) in THF (200 mL) was added dropwise at 30° C. The reaction mixture was stirred at 40° C. under N₂ for 16 hours. The mixture was quenched with H₂O (800 mL). The aqueous phase was extracted by EtOAc (3×500 mL). The combined organic phase was dried over Na₂SO₄, filtered, and concentrated. Purification by silica gel chromatography (PE/EtOAc=100/1 to 20/1) provided H-2.5 (70.0 g). ¹H NMR (400 MHz, CDCl₃) δ_{H} 5.19-5.11 (m, 1H), 3.65-3.51 (m, 1H), 2.51-1.59 (m, 8H), 1.43-1.24 (m, 16H), 1.05 (s, 3H), 0.92 (s, 3H), 0.80 (s, 3H), 0.78-0.65 (m, 1H).

### Synthesis of H-2.6

To a solution of H-2.5 (63.0 g, 199 mmol) in DCM (500 mL) was added 1H-imidazole (20.2 g, 298 mmol) and TBSCI (38.8 g, 258 mmol) at 20° C. The reaction mixture was stirred at 20° C. for 1 hour. The mixture was combined with another batch prepared from 7.00 g of H-2.5 and diluted with DCM (500 mL). The DCM phase was washed with water (2×500 mL) and brine (300 mL), dried over Na₂SO₄, filtered, and concentrated to give the product. Trituration with MeOH (500 mL) provided H-2.6 (50.0 g, 58%). ¹H NMR (400 MHz, CDCl₃) δ_{H} 5.21-5.11 (m, 1H), 3.57-3.51 (m, 1H), 2.25-2.11 (m, 1H), 1.96-1.79 (m, 3H), 1.42-1.22 (m, 12H), 1.04 (s, 3H), 0.98-0.94 (m, 2H), 0.91 (s, 3H), 0.88-0.87 (m, 9H), 0.86-0.57 (m, 9H), 0.06-0.05 (m, 6H).

### Synthesis of H-2.7

To a solution of H-2.6 (45.0 g, 104 mmol) in THF (200 mL) was added BH₃·Me₂S (52 mL, 520 mmol, 10 M) dropwise at 0° C. and the reaction mixture was stirred at 15° C. for 3 hours. The reaction mixture was cooled to 0° C. EtOH (47.8 g, 1.04 mol) was added dropwise at 0° C. NaOH (208 mL, 1.04 mol, 5 M) was added dropwise followed by hydrogen peroxide (104 mL, 1.04 mol, 10.0 M aqueous) at 0° C. The suspension was stirred at 70° C. for 1 hour. The mixture was extracted with EtOAc (3×1 L) and the combined organic phase was washed with saturated aqueous Na₂S₂O₃ (2×500 mL) and brine (300 mL), dried over Na₂SO₄, filtered, and concentrated to give H-2.7 (46.0 g). ¹H NMR (400 MHz, CDCl₃) δ_{H} 4.25-4.24 (m, 1H), 4.15-4.01 (m, 1H), 3.59-3.51 (m, 1H), 1.91-1.56 (m, 10H), 1.56-0.91 (m, 17H), 0.89-0.87 (m, 9H), 0.85-0.59 (m, 7H), 0.06-0.05 (m, 6H).

### Synthesis of H-2.8

To a solution of H-2.7 (41.0 g, 91.3 mmol) in DCM (500 mL) was added silica gel (80.0 g) followed by PCC (39.1 g, 182 mmol) at 20° C. The reaction was stirred at 20° C. for 1 hour. The mixture was filtered and the filter cake was washed with DCM (3×500 mL). The combined DCM solution was evaporated and purified by chromatography (0˜1% EtOAc in PE) to give H-2.8 (12.5 g, 31%). ¹H NMR (400 MHz, CDCl₃) δ_{H} 3.55-3.45 (m, 1H), 2.26-2.24 (m, 1H), 2.14-2.12 (m, 2H), 1.85-1.75 (m, 2H), 1.71-1.61 (m, 3H), 1.46-1.34 (m, 5H), 1.32-1.19 (m, 6H), 1.08-0.99 (m, 3H), 0.92-0.84 (m, 16H), 0.76-0.74 (m, 2H), 0.06-0.05 (m, 9H).

### Synthesis of H-2.9

To a suspension of bromo(methyl)triphenylphosphorane (7.14 g, 20.0 mmol) in THF (20 mL) was added t-BuOK (2.24 g, 20.0 mmol) at 25° C. and the mixture was stirred at 40° C. for 1 hour. H-2.8 (1.50 g, 3.35 mmol) in THF (30 mL) was added at 25° C. and the reaction mixture was stirred at 65° C. for 16 hours. The reaction mixture was quenched with saturated aqueous NH₄Cl (50 mL). The suspension was extracted with EtOAc (3×50 mL). The combined organic phase was washed with brine (80 mL), dried over Na₂SO₄, filtered, and concentrated. Purification by flash chromatography (0%˜3% EtOAc in PE) provided H-2.9 (960 mg, 64%). ¹H NMR (400 MHz, CDCl₃) δ_{H} 4.79 (s, 1H), 4.60 (s, 1H), 3.61-3.48 (m, 1H), 1.90-1.57 (m, 9H), 1.52-0.90 (m, 16H), 0.90-0.89 (m, 1H), 0.88-0.82 (m, 9H), 0.81 (s, 3H), 0.76 (s, 3H), 0.74-0.58 (m, 2H), 0.05 (s, 6H).

### Synthesis of H-2.10

To a solution of H-2.9 (960 mg, 2.15 mmol) in THF (5 mL) was added 9-BBN (17.2 mL, 4.30 mmol, 0.5 M in THF) at 0° C. The reaction mixture was stirred at 0° C. for 2 hours. After cooling to 0° C., EtOH (984 mg, 21.4 mmol) was added drop-wise at 0° C. NaOH (4.28 mL, 21.4 mmol, 5 M) was added dropwise followed by hydrogen peroxide (2.14 mL, 21.4 mmol) at 0° C. The suspension was stirred at 70° C. for 1 hour. The reaction mixture was cooled to 15° C. To the mixture was added water (100 mL) at 15° C. The precipitate was filtered and washed with water (3×50 mL) to give H-2.10 (910 mg, 91%). ¹H NMR (400 MHz, CDCl₃) δ_{H} 3.84-3.52 (m, 1H), 3.59-3.49 (m, 1H), 3.35-3.19 (m, 1H), 1.96-1.59 (m, 7H), 1.52-1.02 (m, 13H), 1.00 (d, J=6.8 Hz, 3H), 0.98-0.90 (m, 2H), 0.89-0.87 (m, 10H), 0.86-0.81 (m, 1H), 0.79 (s, 3H), 0.76 (s, 3H), 0.75-0.57 (m, 3H), 0.05 (s, 6H).

### Synthesis of H-2.11

To a solution of H-2.10 (910 mg, 1.96 mmol) in DCM (15 mL) was added 1-methyl-1H-imidazole (241 mg, 2.94 mmol) and TEA (395 mg, 3.92 mmol) at 20° C. TsCl (747 mg, 3.92 mmol) was added and the reaction mixture was stirred at 20° C. for 2 hours. The mixture was washed with water (2×100 mL) and brine (100 mL), dried over Na₂SO₄, filtered, and concentrated. Purification by flash chromatography (0%˜45% EtOAc in PE) provided H-2.11 (710 mg, 63%). ¹H NMR (400 MHz, CDCl₃) δ_{H} 7.77 (d, J=8.0 Hz, 2H), 7.34 (d, J=8.0 Hz, 2H), 4.15-4.10 (m, 1H), 3.64 (t, J=9.6 Hz, 1H), 3.61-3.53 (m, 1H), 2.45 (s, 3H), 2.15-2.06 (m, 1H), 1.85-1.68 (m, 5H), 1.64-1.47 (m, 5H), 1.44-1.33 (m, 3H), 1.31-0.98 (m, 10H), 0.92 (d, J=7.0 Hz, 3H), 0.89-0.79 (m, 2H), 0.76 (s, 3H), 0.67 (s, 3H), 0.64-0.56 (m, 1H).

### Synthesis of H-2.12

To a solution of H-2.11 (660 mg, 1.31 mmol) in DMF (15 mL) was added KI (869 mg, 5.24 mmol) at 15° C. The mixture was stirred at 55° C. for 48 hours and the iodide H-2.12 was carried to the next step without work-up.

### Synthesis of H-2.13

To the DMF mixture of H-2.12 was added PhSO₂Na 2H₂O (799 mg, 3.90 mmol). The mixture was stirred at 60° C. for 72 hours. The reaction mixture was cooled to 25° C. The mixture was poured into water (20 mL) and filtered. The filter cake was washed with water (3×10 mL). The resulting filter cake was dissolved in DCM (25 mL), washed with water (2×10 mL) and brine (2×10 mL), dried over Na₂SO₄, filtered, and concentrated. Purification by flash chromatography (5%˜20% EtOAc in PE) provided H-2.13 (370 mg, 60%). ¹H NMR (400 MHz, CDCl₃) δ_{H} 7.93-7.88 (m, 2H), 7.68-7.63 (m, 1H), 7.60-7.54 (m, 2H), 3.63-3.53 (m, 1H), 3.22-3.11 (m, 1H), 2.70-2.59 (m, 1H), 2.57-2.47 (m, 1H), 1.84-1.56 (m, 7H), 1.51-0.99 (m, 14H), 0.97-0.77 (m, 5H), 0.75 (s, 3H), 0.73-0.65 (m, 2H), 0.63 (s, 3H), 0.61-0.54 (m, 1H).

### Synthesis of H-2.14

To a solution of H-2.13 (370 mg, 0.783 mmol) in DCM (45 mL) was added DMP (992 mg, 2.34 mmol) at 25° C. After stirring at 25° C. for 2 hours, the mixture was treated with saturated aqueous NaHCO₃ (200 mL) and extracted with DCM (3×100 mL). The combined organic phase was washed with saturated aqueous NaHCO₃ (2×100 mL), saturated aqueous Na₂S₂O₃ (3×100 mL), and brine (150 mL), dried over Na₂SO₄, filtered, and concentrated to give H-2.14 (410 mg, 99%). ¹H NMR (400 MHz, CDCl₃) δ_{H} 7.94-7.89 (m, 2H), 7.68-7.63 (m, 1H), 7.61-7.55 (m, 2H), 3.23-3.17 (m, 1H), 2.78-2.52 (m, 1H), 2.59-2.49 (m, 1H), 2.42-2.19 (m, 3H), 2.11-1.97 (m, 2H), 1.89-1.68 (m, 3H), 1.66-1.62 (m, 1H), 1.52-1.12 (m, 10H), 1.11 (d, J=6.8 Hz, 3H), 0.96 (s, 3H), 0.94-0.69 (m, 6H), 0.68 (s, 3H).

### Synthesis of H-2.15

To a solution of 2,6-di-tert-butyl-4-methylphenol (932 mg, 4.23 mmol) in toluene (3 mL) was added dropwise AlMe; (1.05 mL, 2.11 mmol, 2 M in toluene) at 0° C. After stirring at 25° C. for 1 hour, a solution of H-2.14 (200 mg, 0.424 mmol) in toluene (8 mL) was added dropwise. The mixture was stirred at −70° C. for 30 minutes. To mixture was added EtMgBr (0.846 mL, 2.54 mmol, 3M in ether) dropwise. The resulting mixture was stirred at −70° C. for 1 hour, poured to ice-cooled aqueous citric acid (30 mL) slowly and extracted with EtOAc (3×10 mL). The combined organic layer was washed with brine (30 mL), dried over anhydrous sodium sulfate, filtered, concentrated, and purified by combi-flash (0˜25% EtOAc in PE) to give H-2.15 (80.0 mg, 37%). ¹H NMR (400 MHz, CDCl₃) δ_{H} 7.95-7.85 (m, 2H), 7.70-7.50 (m, 3H), 3.22-3.1 (m, 1H), 2.75-2.62 (m, 1H), 2.58-2.48 (m, 1H), 1.90-1.72 (m, 3H), 1.71-1.65 (m, 1H), 1.56-1.36 (m, 6H), 1.32-1.16 (m, 7H), 1.15-1.05 (m, 7H), 0.96-0.85 (m, 8H), 0.77 (s, 3H), 0.73-0.65 (m, 1H), 0.64-0.55 (m, 4H).

### Synthesis of H-2.16

To THF (0.5 mL) was added n-BuLi (0.568 mL, 2.5 M in hexane, 1.42 mmol). A solution of H-2.15 (80.0 mg, 0.159 mmol) in THF (1.5 mL) was added at -70° C. After stirring at −70° C. for 1 hour, (S)-2-isopropyloxirane (20.4 mg, 0.238 mmol) was added at 70° C. The reaction mixture was stirred at −70° C. for 1 hour and warmed to 15° C. for 16 hours, quenched with sat. aq. NH₄Cl (20 mL) and extracted with EtOAc (2×15 mL). The combined organic layer was dried over Na₂SO₄, filtered, and concentrated to give H-2.16 (90.0 mg), which was carried directly into the next step.

### Synthesis of H-2

To a solution of H-2.16 (90 mg, 0.153 mmol) in MeOH (20 mL) was added NiCl₂ (3.96 mg, 0.031 mmol) and Mg powder (146 mg, 6.12 mmol) at 65° C. in one portion. After stirring at 65° C. for 10 minutes, another portion of Mg powder (74 mg, 3.06 mmol) was added at 65° C. The mixture was stirred at 65° C. for another 10 minutes, quenched with HCl (30 mL, 2N) until the reaction became clear and extracted with EtOAc (3×10 mL). The combined organic layer was washed with sat. aq. NH₄Cl (30 mL), dried over Na₂SO₄, filtered, concentrated, and purified by silica gel chromatography (0˜15% of EtOAc in PE) two times to give H-2 (3.00 mg, 4%). ¹H NMR (400 MHz, CDCl₃) δ_{H} 3.38-3.26 (m, 1H), 2.01-1.60 (m, 9H), 1.56-1.13 (m, 20H), 1.12-1.00 (m, 3H), 0.99-0.83 (m, 13H), 0.82-0.61 (m, 8H). LC-ELSD/MS 30-90AB_2 min_E, purity >99%, MS ESI calcd. for C₃₀H₅₄O₂ [M+H-2H₂O]⁺ 411.4, found 411.4.

### Example 79: Synthesis of (2S,4aS,4bS,6aR,7R,10aS,10bR,12aS)-7-((R)-4-(1-hydroxy-4,4-dimethylcyclohexyl) butan-2-yl)-4a,6a-dimethyl-2 (trifluoromethyl)octadecahydrochrysen-2-ol (H-3)

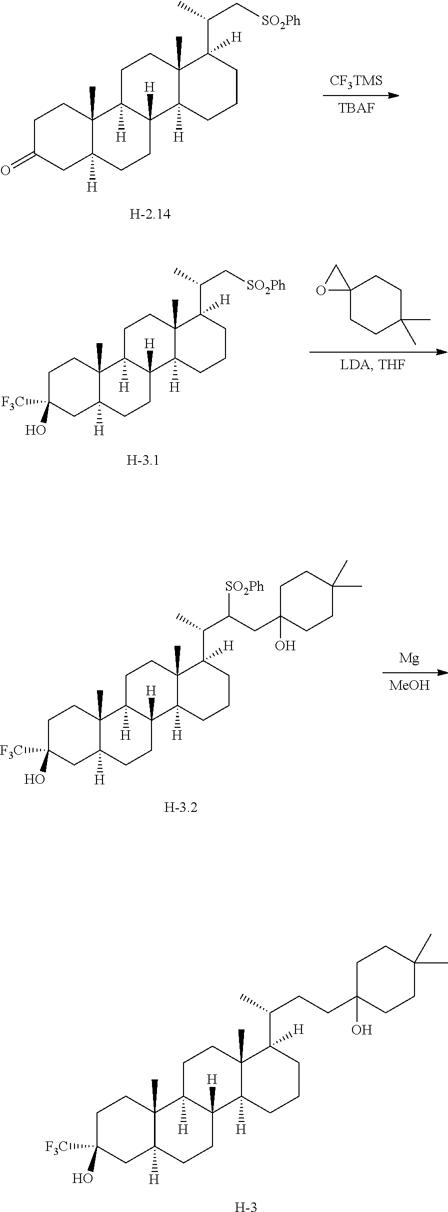

### Synthesis of H-3.1

To a solution of H-2.14 (200 mg, 0.425 mmol) in THF (10 mL) was added CsF (256 mg, 1.69 mol) and TMSCF₃ (240 mg, 1.69 mol) at 0° C. The mixture was stirred at 0° C. for 1 hour. To the mixture was added TBAF (1.69 mL, 1 M in THF, 1.69 mmol) at 25° C. and stirring was continued at 25° C. for 1 hour. The mixture was concentrated, and the residue was dissolved in EtOAc (100 mL), washed with water (2×60 mL) and brine (100 mL), dried over Na₂SO₄, filtered, and concentrated to give the product. Purification by flash-chromatography (10% EtOAc in PE) provided H-3.1 (167 mg, 73%). ¹H NMR (400 MHz, CDCl₃) δ_{H} 7.93-7.88 (m, 2H), 7.68-7.62 (m, 1H), 7.60-7.54 (m, 2H), 3.22-3.17 (m, 1H), 2.76-2.45 (m, 1H), 2.57-2.48 (m, 1H), 2.07-2.06 (m, 1H), 1.99-1.97 (m, 1H), 1.84-1.58 (m, 7H), 1.53-1.05 (m, 14H), 0.98-0.81 (m, 3H), 0.79 (s, 3H), 0.77-0.65 (m, 3H), 0.64 (s, 3H).

### Synthesis of H-3.2

To THF (0.5 mL) was added n-BuLi (0.284 mL, 2.5 M in hexanes, 0.710 mmol) at 70° C. Then a solution of H-3.1 (167 mg, 0.309 mmol) in THF (1 mL) was added at −70° C. After stirring at -70° C. for 1 hour, 6,6-dimethyl-1-oxaspiro[2.5]octane (64.9 mg, 0.463 mmol) was added at −70° C. The reaction mixture was stirred at 25° C. for 16 hours. The reaction mixture was quenched with water (50 mL) and extracted with EtOAc (3×30 mL). The combined organic phase was washed with brine (60 mL), dried over Na₂SO₄, filtered, and concentrated to give H-3.2 (200 mg), which was used directly in the next step.

### Synthesis of H-3

To a solution of H-3.2 (200 mg, 0.294 mmol) and NiCl₂ (7.61 mg, 0.059 mmol) in dry MeOH (20 mL) was added Mg powder (284 mg, 11.7 mmol) in 2 portions under N₂ with stirring at 65° C. The reaction mixture was stirred at 65° C. for 1 hour. The reaction mixture was cooled and poured into EtOAc (150 mL). The mixture was washed with 1 M HCl (1×100 mL), saturated aqueous NaHCO₃ (100 mL), and brine (100 mL), dried over Na₂SO₄, filtered, and concentrated to give the product, which was purified by silica gel flash-chromatography (10% EtOAc in PE) to afford H-3 (20 mg, 13%). ¹H NMR (400 MHz, CDCl₃) δ_{H} 2.08-2.04 (m, 1H), 1.99-1.97 (m, 1H), 1.93-1.75 (m, 4H), 1.71-1.58 (m, 4H), 1.53-1.51 (m, 2H), 1.50-1.47 (m, 2H), 1.46-1.42 (m, 5H), 1.38-1.05 (m, 13H), 0.91-0.89 (m, 1H), 0.93 (s, 3H), 0.92-0.89 (m, 1H), 0.88-0.85 (m, 6H), 0.84-0.83 (m, 2H), 0.82 (s, 3H), 0.79 (s, 3H), 0.77-0.66 (m, 4H). ¹⁹F NMR (376 MHz, CDCl₃) δ_{F} −78.66 (s) LC-ELSD/MS 30-90AB_2 min_E, purity >99%, MS ESI calcd. for C33HssF₃O₂ [M−H₂O+H]⁺ 523.4115, found 523.4115.

### Example 80: Synthesis of (2S,4aS,4bS,6aR,7R,10aS,10bR,12aS)-2-ethyl-7-((R)-6-hydroxy-6-methylheptan-2-yl)-4a,6a-dimethyloctadecahydrochrysen-2-ol (H-4)

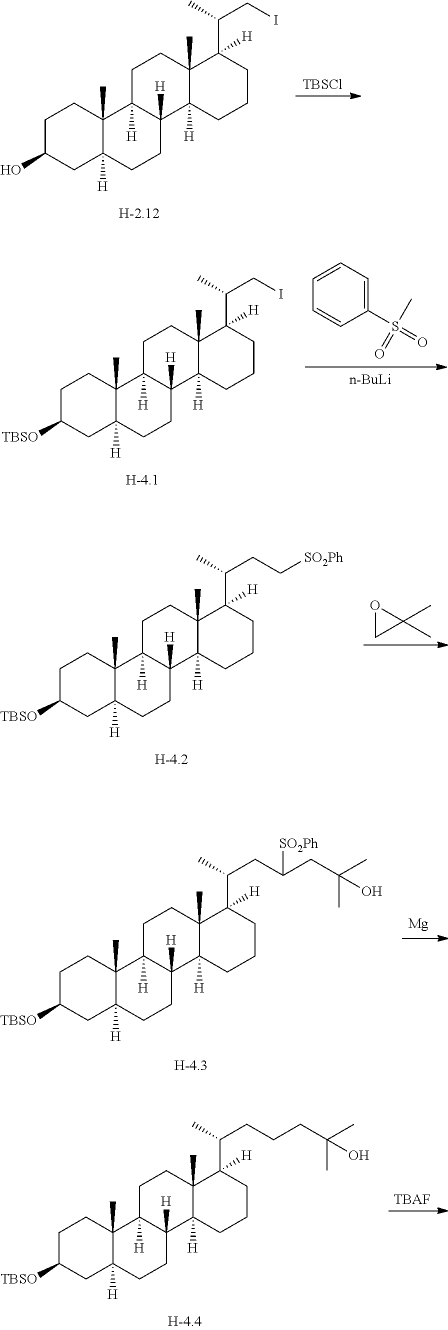

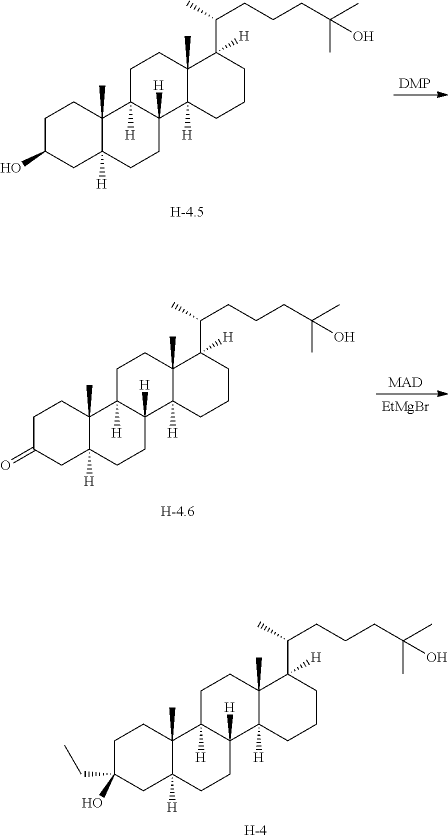

### Synthesis of H-4.1

To a solution of H-2.12 (1.20 g, 2.61 mmol) and imidazole (354 mg, 5.22 mmol) in DCM (20 mL) was added TBSCI (786 mg, 5.22 mmol). The mixture was stirred at 20° C. for 16 hours. To the reaction mixture was added water (50 mL) and the mixture was extracted with DCM (20 mL). The combined organic layers were washed with brine (20 mL), dried over Na₂SO₄, filtered, and concentrated to give H-4.1 (1.05 g). ¹H NMR (400 MHz, CDCl₃) δ_{H} 3.57-3.51 (m, 1H), 3.46-3.42 (m, 1H), 2.78-2.74 (m, 1H), 2.25-2.21 (m, 1H), 1.89-1.59 (m, 8H), 1.47-1.43 (m, 2H), 1.31-1.01 (m, 15H), 0.89-0.81 (m, 9H), 0.80 (s, 3H), 0.76 (s, 3H), 0.75-0.73 (m, 3H), 0.06-0.05 (m, 6H).

### Synthesis of H-4.2

To a solution of (methylsulfonyl)benzene (3.24 g, 20.8 mmol) in THF (25 mL) was added n-BuLi (6.95 mL, 17.4 mmol, 2.5 M in hexane) at -70° C. under N₂. The mixture was stirred at −70° C. for 30 minutes. A solution of H-4.1 (1.00 g, 1.74 mmol) in THF (25 mL) was added dropwise at 25° C. After the addition, the reaction was stirred at 25° C. for 16 hours. The reaction was quenched with sat. Aq. NH₄Cl (50 mL) and extracted with EtOAc (3×50 mL). The combined organic phase was dried over Na₂SO₄, filtered, and concentrated. The residue was purified by combi flash (0˜10% EtOAc in PE) to give H-4.2 (602 mg, 60%). ¹H NMR (400 MHz, CDCl₃) δ_{H} 7.92-7.89 (m, 2H), 7.68-7.63 (m, 1H), 7.59-7.55 (m, 2H), 3.53-3.51 (m, 1H), 3.21-3.08 (m, 1H), 2.95-2.85 (m, 1H), 1.90-1.59 (m, 7H), 1.51-0.94 (m, 14H), 1.05-0.95 (m, 3H), 091-0.88 (m, 9H), 0.86-0.83 (m, 3H), 0.80-0.74 (m, 3H), 0.72-0.66 (m, 2H), 0.63 (s, 3H), 0.61-0.58 (m, 2H), 0.05-0.04 (m, 6H).

### Synthesis of H-4.3

To THF (0.8 mL) was added n-BuLi (0.8 mL, 2.5 M in hexanes, 2.00 mmol) at 70° C. To the mixture was added H-4.2 (602 mg, 1.00 mmol) in THF (5 mL) at −70° C. The mixture was stirred at −70° C. for 1 hour. Then 2,2-dimethyloxirane (108 mg, 1.50 mmol) was added at -70° C. The reaction mixture was stirred at 20° C. for 16 hours. The reaction mixture was quenched with water (50 mL) and extracted with EtOAc (3×50 mL). The combined organic phase was washed with brine (60 mL), dried over Na₂SO₄, filtered, and concentrated to give H-4.3 (500 mg). ¹H NMR (400 MHz, CDCl₃) δ_{H} 7.94-7.87 (m, 2H), 7.70-7.60 (m, 1H), 7.59-7.52 (m, 2H), 3.56-3.49 (m, 1H), 3.41-3.34 (m, 1H), 3.28-3.22 (m, 1H), 2.48-2.39 (m, 1H), 2.25-2.15 (m, 1H), 1.85-1.61 (m, 8H), 1.54-1.41 (m, 10H), 1.38-1.31 (m, 3H), 1.28-1.07 (m, 15H), 0.91-0.81 (m, 10H), 0.80-0.51 (m, 12H).

### Synthesis of H-4.4

To a solution of H-4.3 (500 mg, 0.742 mmol) in dry MeOH (20 mL) and THF (15 mL) was added Mg powder (1.20 g, 49.9 mmol) under N₂ at 65° C. The reaction was stirred at 60° C. for 1 hour. The reaction mixture was quenched with 1 M HCl (50 mL) and extracted with DCM (2×100 mL). The organic layer was washed with sat. aq. NaHCO₃ (100 mL) and brine (100 mL), dried over Na₂SO₄, filtered, and concentrated to give H-4.4 (300 mg). 1H NMR (400 MHz, CDCl₃) δ_{H} 3.59-3.51 (m, 1H), 1.89-1.66 (m, 13H), 1.54-1.27 (m, 16H), 1.25-1.21 (m, 4H), 1.18-1.11 (m, 4H), 0.91-0.81 (m, 9H), 0.76 (s, 3H), 0.74-0.70 (m, 4H), 0.69-0.64 (m, 4H), 0.05-0.04 (m, 6H).

### Synthesis of H-4.5

To a solution of H-4.4 (300 mg, 0.562 mmol) in THF (5 mL) was added TBAF (292 mg, 1.12 mmol) and the mixture was stirred at 70° C. for 16 hours. The reaction mixture was added to saturated aqueous NH₄Cl (20 mL) and extracted with EtOAc (3×50 mL). The combined organic layer was dried over Na₂SO₄, filtered, and concentrated to give the product. Purification by combi flash (EtOAc/PE=0-20%) provided H-4.5 (140 mg, 60%). 1H NMR (400 MHz, CDCl₃) δ_{H} 3.59-3.51 (m, 1H), 1.88-1.61 (m, 6H), 1.48-1.25 (m, 12H), 1.24 (s, 3H), 1.22-0.98 (m, 10H), 0.96-0.81 (m, 12H), 0.77 (s, 3H), 0.75-0.62 (m, 3H).

### Synthesis of H-4.6

To a mixture of DMP (283 mg, 0.668 mmol) in DCM (10 mL) was added H-4.5 (140 mg, 0.334 mmol) in DCM (10 mL) at 20° C. To the mixture was added H₂O (0.1 mL) at 20° C. and the reaction mixture was stirred at 20° C. for 1 hour. The reaction mixture was quenched with saturated aqueous NaHCO₃ (50 mL) at 20° C. and the mixture was filtered. The organic phase was separated and washed with saturated aqueous NaHCO₃/Na₂S₂O₃ (1:1, 2×40 mL) and brine (50 mL), dried over anhydrous Na₂SO₄, filtered, and concentrated. The product was purified by flash column chromatography (0˜20% EtOAc in PE) to give H-4.6 (130 mg, 94%). ¹H NMR (400 MHz, CDCl₃) δ_{H} 2.41-2.24 (m, 4H), 2.12-1.61 (m, 12H), 1.48-1.24 (m, 14H), 0.99 (s, 3H), 0.97-0.81 (m, 9H), 0.79 (s, 3H), 0.77-0.71 (m, 3H).

### Synthesis of H-4

To a solution of BHT (1.32 g, 5.99 mmol) in toluene (3 mL) under nitrogen at 0° C. was added trimethylaluminum (1.49 mL, 2 M, 2.99 mmol) dropwise. The mixture was stirred at 0° C. for 30 minutes and used directly as a solution of MAD (0.5 M in toluene) without further purification. To the solution of MAD (1.86 mL, 0.5 M, 0.933 mmol) under N₂ at 78° C. was added a solution of H-4.6 (130 mg, 0.311 mmol) in toluene (10 mL) dropwise. The mixture was stirred at −78° C. for 30 minutes. To the above mixture was added EtMgBr (3 M in diethyl ether, 0.311 mL, 0.933 mmol, 3M) dropwise. The resulting mixture was stirred at 78° C. for 0.5 hour. The reaction mixture was poured to ice-cooled aqueous citric acid (20 mL) and extracted with EtOAc (2×50 mL). The combined organic layer was washed with brine (50 mL), dried over anhydrous Na₂SO₄, filtered, and concentrated. The residue was purified by combi-flash (0˜10% EtAOc in PE) to give H-4 (14.0 mg, 10%). ¹H NMR (400 MHz, CDCl₃) δ_{H} 1.88-1.59 (m, 8H), 1.48-1.26 (m, 17H), 1.25-1.23 (m, 6H), 1.12-0.99 (m, 3H), 0.97-0.82 (m, 11H), 0.80 (s, 3H), 0.77 (s, 3H), 0.76-0.73 (m, 3H). LC-ELSD/MS 30-90AB_2 min_E, purity >99%, MS ESI calcd. for C₃₀H₅₄O₂ [M+H-2H₂O]⁺ 411.4, found 411.4.

### Example 81: Synthesis of (2S,4aR,4bS,6aR,7R,10aS,10bS)-2-ethyl-7-((2R,5S)-5-hydroxy-6-methylheptan-2-yl)-4a,6a-dimethyl-1,2,3,4,4a,4b,5,6,6a,7,8,9,10,10a,10b,11-hexadecahydrochrysen-2-ol (H-5) and (2S,4aR,4bS,6aR,7R,10aS,10bS)-2-ethyl-7-((2R,5R)-5-hydroxy-6-methylheptan-2-yl)-4a,6a-dimethyl-1,2,3,4,4a,4b,5,6,6a,7,8,9,10,10a,10b,11-hexadecahydrochrysen-2-ol (H-6)

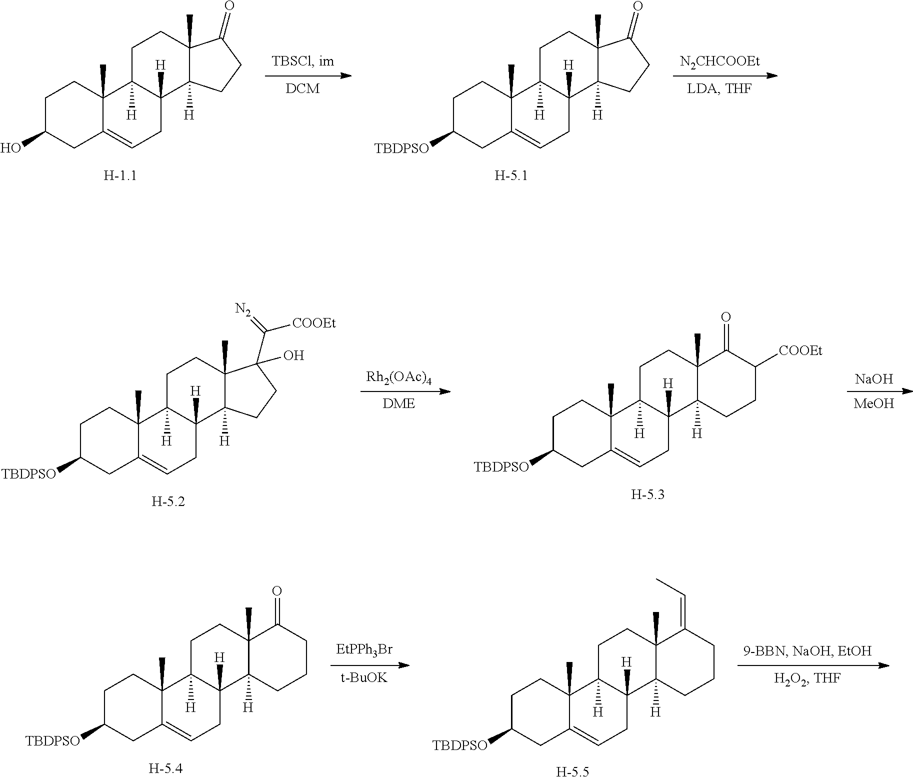

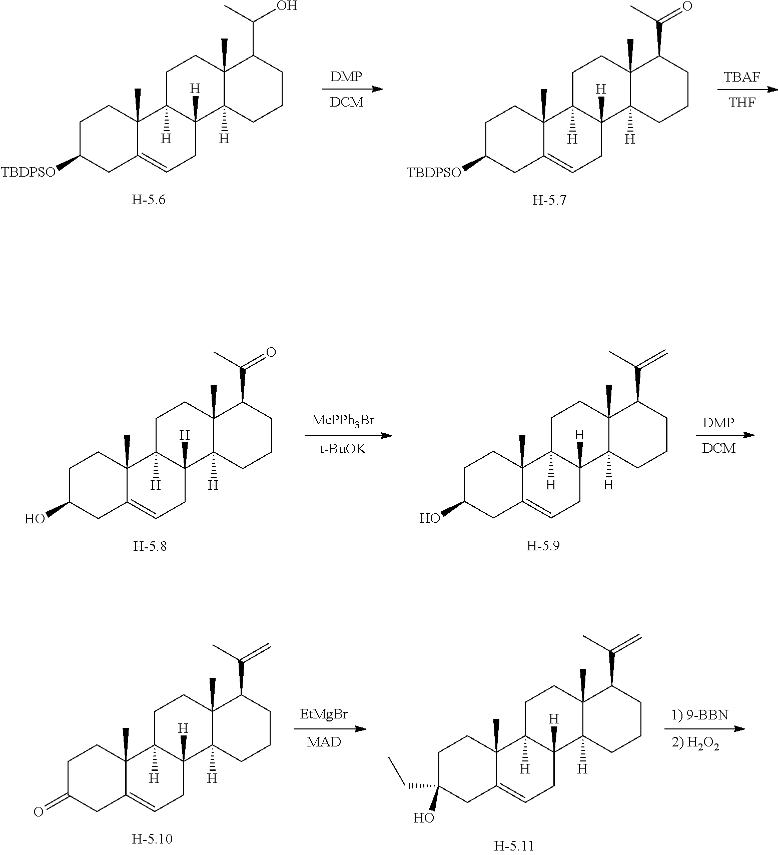

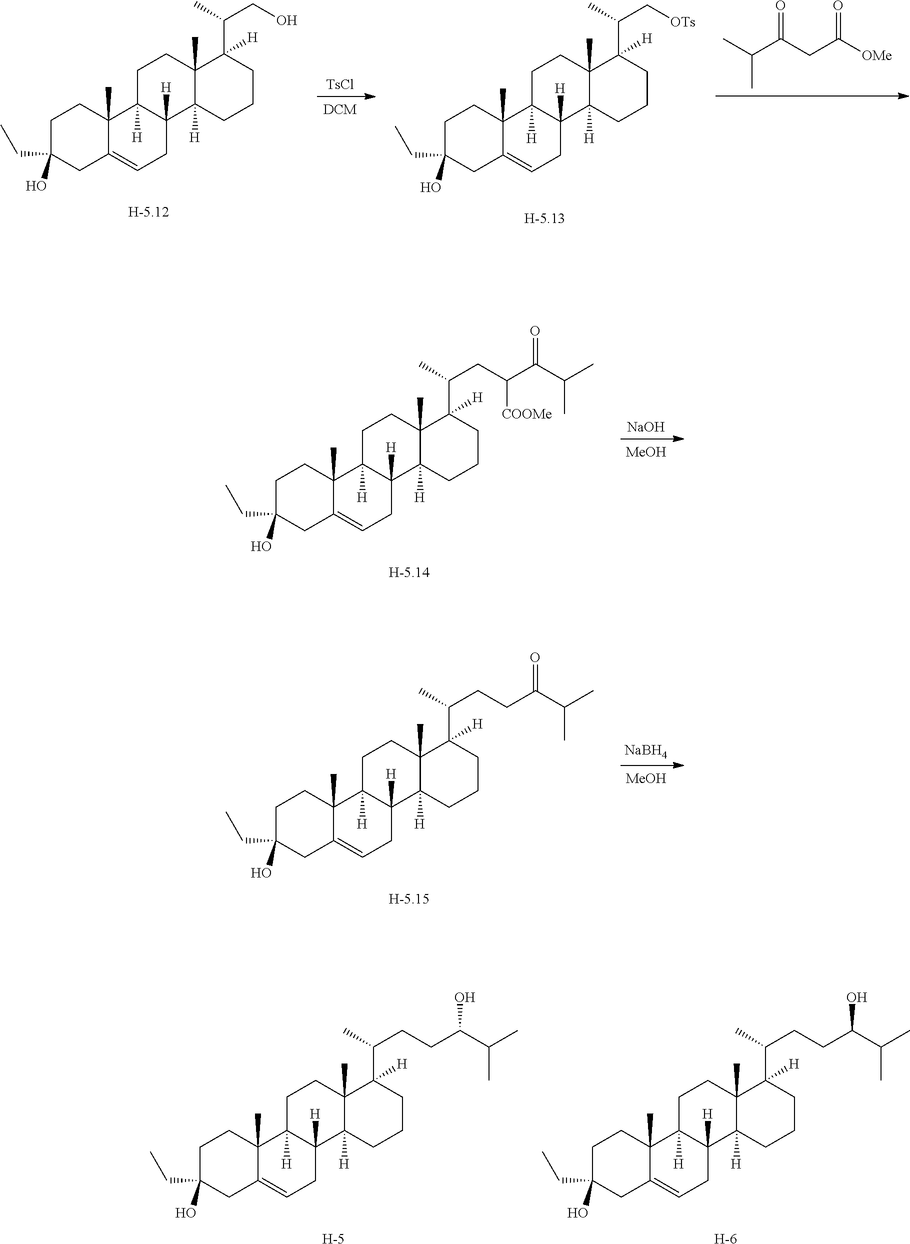

### Synthesis of H-5.1

To a solution of H-1.1 (50 g, 182 mmol) in DCM (400 mL) was added imidazole (24.7 g, 364 mmol) and TBDPSCI (70.7 mL, 273 mmol, 1.057 g/mL) at 25° C. and the resulting mixture was stirred for 24 h. The mixture was poured into water (600 mL) and extracted with DCM (2×200 mL). The combined organic layers were washed with brine (2×400 mL), dried over anhydrous Na₂SO₄, filtered, and concentrated. The residue was triturated from MeOH (250 mL) at 25° C. to give H-5.1 (88 g). ¹H NMR (400 MHz, CDCl₃) δ_{H} 7.71-7.65 (m, 4H), 7.41-7.33 (m, 6H), 5.18-5.10 (m, 1H), 3.60-3.44 (m, 1H), 2.53-2.27 (m, 2H), 2.20-1.96 (m, 3H), 1.95-1.86 (m, 1H), 1.84-1.77 (m, 1H), 1.75-1.65 (m, 2H), 1.65-1.57 (m, 3H), 1.55-1.37 (m, 3H), 1.27-1.17 (m, 2H), 1.10-1.04 (m, 9H), 1.01 (s, 3H), 0.93-0.82 (m, 5H).

### Synthesis of H-5.2

A cold (−70° C.) LDA solution (142 mL, 284 mmol, 2 M) was added to a stirred solution of H-5.1 (30 g, 56.9 mmol) and ethyl diazoacetate (29.8 mL, 284 mmol) in THF (600 mL) at −70° C. and the resulting mixture was stirred for 4 h. Acetic acid (16.1 mL, 284 mmol) in THF (10 mL) was added and the mixture was warmed to 25° C. and stirred for 16 h. The mixture was poured into H₂O (500 mL) and extracted with EtOAc (200 mL×3). The combined organic layers were washed with brine (100 mL×2), dried over anhydrous Na₂SO₄, filtered, and concentrated to give H-5.2 (36 g).

### Synthesis of H-5.3

To a solution of H-5.2 (59 g, 92.0 mmol) in DME (600 mL) was added Rh₂(OAc)₄ (813 mg, 1.84 mmol) and the resulting mixture was stirred at 25° C. for 16 h. The reaction mixture was concentrated to give H-5.3 (50 g).

### Synthesis of H-5.4

To a mixture of H-5.3 (50 g, 81.5 mmol) in MeOH (500 mL) was added NaOH (26.0 g, 652 mmol, in H₂O (125 mL)) and the resulting mixture was stirred at 60° C. for 16 h. The reaction mixture was concentrated and H₂O (600 mL) was added. The mixture was extracted with EtOAc (2×600 mL) and the combined organic layers were washed with brine (2×600 mL), dried over anhydrous Na₂SO₄, filtered, and concentrated to give H-5.4 (45 g). ¹H NMR (400 MHz, CDCl₃) δ_{H} 7.70-7.65 (m, 4H), 7.40-7.35 (m, 6H), 5.16-5.09 (m, 1H), 4.24-4.01 (m, 1H), 3.57-3.48 (m, 1H), 2.68-2.57 (m, 1H), 2.36-2.28 (m, 1H), 2.26-2.07 (m, 3H), 1.79-1.64 (m, 5H), 1.64-1.58 (m, 3H), 1.53-1.49 (m, 1H), 1.46-1.30 (m, 4H), 1.08 (s, 3H), 1.05 (s, 9H), 0.98 (s, 3H), 0.81-0.88 (m, 2H).

### Synthesis of H-5.5

To a mixture of EtPPh₃Br (123 g, 332 mmol) in THF (500 mL) was added t-BuOK (37.1 g, 332 mmol) at 20° C. under N₂ and the resulting mixture was stirred at 50° C. for 30 min. H-5.4 (30 g, 55.4 mmol) in THF (100 mL) was added in portions to keep the internal temperature below 50° C. The reaction mixture was stirred for 16 h. The reaction mixture was quenched with 10% aqueous NH₄Cl (200 mL) at 25° C. and the aqueous layer was extracted with EtOAc (3×300 mL). The combined organic layers were washed with brine (2×300 mL), dried over anhydrous Na₂SO₄, filtered, and concentrated to give H-5.5 (125 g).

### Synthesis of H-5.6

To a solution of H-5.5 (80 g, 144 mmol) in THF (1 L) was added 9-BBN dimer (52.7 g, 216 mmol) and the resulting mixture was stirred at 25° C. under N₂ for 16 h. To the reaction mixture was added ethanol (74.1 mL, 1.29 mol) and NaOH (258 mL, 5 M, 1.29 mol). H₂O₂ (129 mL, 10 M, 1.29 mol) was added dropwise at 25° C. and the temperature was raised to reflux (75° C.). The mixture was cooled and stirred for 16 h. To the mixture was added Na₂SO₃ (600 mL, 20% aq.) and water (1000 mL) at 25° C. and the mixture was stirred for 1 h. After the stirrer was turned off, a clear lower layer and an upper suspension layer were formed. The clear lower layer was discarded. To the upper suspension layer was added water (600 mL) and the mixture was stirred for 15 min. The mixture was filtered and the solid was washed with water. The residue was triturated from MeOH/H₂O (1:1, 1.5 L) to give H-5.6 (40 g). ¹H NMR (400 MHz, CDCl₃) δ_{H} 7.70-7.64 (m, 4H), 7.45-7.32 (m, 6H), 5.20-5.05 (m, 2H), 3.57-3.48 (m, 1H), 2.55-2.42 (m, 1H), 2.37-2.24 (m, 1H), 2.18-2.10 (m, 1H), 1.99-1.73 (m, 6H), 1.62-1.53 (m, 6H), 1.50-1.30 (m, 6H), 1.15-1.12 (m, 1H), 1.05 (s, 9H), 0.97 (s, 3H), 0.91 (s, 3H), 0.88-0.63 (m, 4H).

### Synthesis of H-5.7

To a solution of H-5.6 (50 g, 87.5 mmol) in DCM (800 mL) at 25° C. was added DMP (74.2 g, 175 mmol) and the resulting mixture was stirred for 2 h. Saturated aqueous NaHCO₃ (1000 mL) was added dropwise along with saturated aqueous Na₂S₂O₃ (1000 mL) and the mixture was stirred at 25° C. for 10 min. The resulting mixture was washed with DCM (400 mL) and the filtrate was concentrated to give H-5.7 (40 g).

### Synthesis of H-5.8

To a suspension of H-5.7 (50 g, 87.8 mmol) in THF (400 mL) was added TBAF (175 mL, 175 mmol) and the resulting mixture was stirred at 25° C. for 16 h. The mixture was quenched with water (200 mL) and extracted with EtOAc (3×200 mL). The combined organic layers were washed with brine (2×200 mL), dried over anhydrous Na₂SO₄, filtered, and concentrated. The residue was triturated from ethyl acetate and purified by silica gel chromatography (0˜15% EtOAc in PE) to give H-5.8 (41 g).

### Synthesis of H-5.9

To a mixture of MePPh₃Br (16.1 g, 45.3 mmol) in THF (70 mL) was added t-BuOK (5.08 g, 45.3 mmol) at 25° C. under N₂ and the resulting mixture was stirred at 55° C. for 30 min. H-5.8 (5 g, 15.1 mmol) in THF (30 mL) was added in portions to keep the internal temperature below 55° C. and the reaction mixture was stirred for 16 h. The reaction mixture was quenched with 10% aqueous NH₄Cl (100 mL), extracted with EtOAc (3×100 mL), washed with brine (2×150 mL), dried over anhydrous Na₂SO₄, filtered, concentrated and was triturated from MeOH/H₂O (0.5 L, 1:1). The residue was purified by silica gel chromatography (0˜15% of EtOAc in PE) to give H-5.9 (5 g). ¹H NMR (400 MHz, CDCl₃) δ_{H} 5.41-5.25 (m, 1H), 4.80 (s, 1H), 4.65-4.55 (m, 1H), 3.60-3.45 (m, 1H), 2.34-2.19 (m, 2H), 2.18-2.10 (m, 1H), 1.92-1.73 (m, 4H), 1.73-1.58 (m, 5H), 1.52-1.35 (m, 6H), 1.32-1.15 (m, 3H), 1.12-0.91 (m, 8H), 0.88-0.75 (m, 3H).

### Synthesis of H-5.10

To a solution of H-5.9 (7 g, 21.3 mmol) in DCM (100 mL) was added DMP (13.5 g, 31.9 mmol) and the resulting mixture was stirred at 20° C. for 2 h. The mixture was washed with a solution of NaHCO₃ (100 mL, aq. sat.) and Na₂S₂O₃ (100 mL, aq. sat.) twice, dried over anhydrous Na₂SO₄, filtered, and concentrated to give H-5.10 (7 g).

### Synthesis of H-5.11

To a solution of BHT (28.8 g, 130 mmol) in toluene (60 mL) under nitrogen at 0° C. was added AlMe₃ (2 M in toluene, 32.5 mL 65.0 mmol) dropwise. The resulting mixture was stirred at 15° C. for 1 h and used directly as a solution of MAD without further purification. To the MAD solution (64.1 mmol in 200 mL toluene) was added a solution of H-5.10 (7 g, 21.4 mmol) in anhydrous DCM (20 mL) dropwise at −70° C. After stirring at −70° C. for 1 h under N₂, MeMgBr (21.3 mL, 64.1 mmol, 3M in ethyl ether) was added dropwise at −70° C. The resulting solution was stirred at −70° C. for 3 h. The reaction mixture was poured into citric acid (100 mL, 20% aq.) below 10° C. and extracted with EtOAc (2×120 mL). The combined organic layers were dried over anhydrous Na₂SO₄, filtered, and concentrated. PE (100 mL) was added and the mixture was stirred for 10 min. The mixture was filtered and the filtrate was concentrated and then purified by silica gel chromatography (0˜20% of EtOAc in PE) to give H-5.11 (4 g). ¹H NMR (400 MHz, CDCl₃) δ_{H} 5.38-5.19 (m, 1H), 4.87-4.78 (m, 1H), 4.65-4.58 (m, 1H), 2.38-2.31 (m, 1H), 2.17-2.10 (m, 1H), 2.03-1.98 (m, 1H), 1.78-1.70 (m, 7H), 1.60-1.56 (m, 2H), 1.54-1.46 (m, 3H), 1.45-1.42 (m, 2H), 1.41-1.34 (m, 3H), 1.23-1.14 (m, 2H), 1.12-1.06 (m, 4H), 1.00 (s, 3H), 0.97-0.90 (m, 2H), 0.89-0.80 (m, 6H).

### Synthesis of H-5.12

To a solution of H-5.11 (5 g, 14.0 mmol) in THF (100 mL) was added 9-BBN dimer (10.2 g, 42.0 mmol) and the resulting mixture was stirred at 25° C. under N₂ for 16 h. To the mixture was added ethanol (8.16 mL, 140 mmol) at 25° C., followed by aqueous NaOH (28.0 mL, 5.0 M, 140 mmol) at 0° C. Hydrogen peroxide (14.0 mL, 10 M, 140 mmol) was added dropwise at 0° C. and the reaction mixture was stirred at 80° C. for 1 h. The mixture was cooled to 25° C. and Na₂SO₃ (100 mL, sat. aq.) was added. The aqueous layer was extracted with EtOAc (3×50 mL) and the combined organic layers were washed brine (2×50 mL), dried over anhydrous Na₂SO₄, filtered, and concentrated in vacuo. The residue was purified by silica gel chromatography (0˜25% of EtOAc in PE) to give H-5.12 (5 g).

### Synthesis of H-5.13

To a solution of H-5.12 (5 g, 13.3 mmol) in DCM (70 mL) was added 1-methyl-1H-imidazole (5.27 mL, 66.5 mmol) and TsCl (5.07 g, 26.6 mmol) and the resulting mixture was stirred at 20° C. for 16 h. The mixture was poured into saturated H₂O (100 mL) and the aqueous layer was extracted with DCM (3×80 mL). The combined organic layers were washed with brine (100 mL), dried over anhydrous Na₂SO₄, filtered, concentrated, and purified by silica gel chromatography (0˜15% EtOAc in PE) to give H-5.13 (2 g). The residue (1 g, 1.97 mmol) was purified by SFC (DAICEL CHIRALPAK AD (250 mm*50 mm, 10 um), Condition: 0.1% NH₃H₂O ETOH, Begin B: 30%, End B: 30%, FlowRate (ml/min): 80, Injections: 240) to give H-5.13 (1 g). ¹H NMR (400 MHz, CDCl₃) δ_{H} 7.78 (d, J=8.0 Hz, 2H), 7.34 (d, J=8.0 Hz, 2H), 5.29-5.23 (m, 1H), 4.19-4.07 (m, 1H), 3.69-3.60 (m, 1H), 2.45 (s, 3H), 2.36-2.28 (m, 1H), 2.16-1.98 (m, 3H), 1.81-1.68 (m, 3H), 1.67-1.58 (m, 3H), 1.51-1.26 (m, 8H), 1.17-1.03 (m, 3H), 1.02-0.96 (m, 4H), 0.96-0.90 (m, 5H), 0.88-0.80 (m, 4H), 0.74-0.65 (m, 4H).

### Synthesis of H-5.14

To a solution of NaH (105 mg, 60%, 2.64 mmol) in dioxane (4 mL) was added methyl 4-methyl-3-oxopentanoate (490 mg, 3.40 mmol) at 0° C. under N₂ and the resulting mixture was stirred for 1 h. DMF (2 mL) and H-5.13 (200 mg, 0.378 mmol) in dioxane (2 mL) were added dropwise, and the reaction mixture was stirred at 105° C. for 16 h. The mixture was poured into saturated aqueous NH₄Cl (10 mL) and the aqueous phase was extracted with DCM (3×20 mL). The combined organic layers were washed with water (2×20 mL), dried over anhydrous Na₂SO₄, filtered, and concentrated to give H-5.14 (200 mg).

### Synthesis of H-5.15

To a mixture of H-5.14 (200 mg, 0.410 mmol) in MeOH (10 mL) was added H₂O (2 mL) and NaOH (81.9 mg, 2.05 mmol) at 25° C. and the resulting mixture was stirred at 20° C. for 16 h. The mixture was extracted with DCM (4×10 mL) and the combined organic layers were washed with brine (20 mL), dried over anhydrous Na₂SO₄, filtered, and concentrated. The residue was purified by silica gel chromatography (0-15% of EtOAc in PE) to give H-5.15 (100 mg). ¹H NMR (400 MHz, CDCl₃) δ_{H} 5.36-5.14 (m, 1H), 2.64-2.55 (m, 1H), 2.54-2.42 (m, 1H), 2.40-2.30 (m, 2H), 2.17-2.07 (m, 1H), 2.06-2.00 (m, 1H), 1.93-1.86 (m, 1H), 1.84-1.70 (m, 4H), 1.66-1.60 (m, 2H), 1.48-1.30 (m, 8H), 1.29-1.18 (m, 4H), 1.11-1.06 (m, 7H), 1.00 (s, 3H), 0.96-0.90 (m, 3H), 0.88-0.81 (m, 10H), 0.77-0.68 (m, 1H).

### Synthesis of H-5 and H-6

To a solution of H-5.15 (40 mg, 0.0903 mmol) in MeOH (3 mL) at 0° C. was added NaBH₄ (41.0 mg, 1.08 mmol) and the resulting mixture was stirred for 1 h. The mixture was poured into saturated aqueous NH₄Cl (5 mL) and extracted with DCM (2×10 mL). The combined organic layers were washed with brine (2×10 mL), dried over anhydrous Na₂SO₄, filtered, and concentrated in vacuo. The residue was purified by silica gel chromatography (0˜15% of EtOAc in PE), and was further purified by SFC (DAICEL CHIRALPAK AD (250 mm*30 mm, 10 um), Condition: 0.1% NH₃H₂O IPA, Begin B: 30%, End B: 30%, FlowRate (mL/min): 70, Injections: 70) and lyophilized to give H-5 (4.2 mg, 10.5%) and H-6 (4.2 mg, 10.5%).

H-5: ¹H NMR (400 MHz, CDCl₃) δ_{H} 5.32-5.22 (m, 1H), 3.35-3.22 (m, 1H), 2.39-2.27 (m, 1H), 2.17-2.00 (m, 2H), 1.98-1.87 (m, 1H), 1.84-1.68 (m, 4H), 1.54-1.30 (m, 11H), 1.29-1.11 (m, 5H), 1.10-1.04 (m, 1H), 1.03-0.97 (m, 4H), 0.95-0.78 (m, 20H), 0.75-0.70 (m, 1H). LC-ELSD/MS 30-90AB_2 min_E, purity>99%, MS ESI calcd. for C₃₀H₅₁O [M−H₂O+H]⁺ 427.4, found 427.4.

H-6: ¹H NMR (400 MHz, CDCl₃) δ_{H} 5.31-5.22 (m, 1H), 3.40-3.21 (m, 1H), 2.40-2.30 (m, 1H), 2.18-2.08 (m, 1H), 2.07-2.00 (m, 1H), 1.97-1.88 (m, 1H), 1.84-1.68 (m, 4H), 1.52-1.15 (m, 16H), 1.14-1.03 (m, 2H), 1.02-0.97 (m, 4H), 0.97-0.84 (m, 15H), 0.83-0.76 (m, 4H), 0.75-0.68 (m, 1H). LC-ELSD/MS 30-90AB_2 min_E, purity>99%, MS ESI calcd. for C₃₀H₅₁O [M−H₂O+H]⁺ 427.4, found 427.4.

### Example 82: Synthesis of (2S,4aR,4bS,6aR,7R,10aS,10bS)-7-((2R,5S)-5-hydroxy-6-methylheptan-2-yl)-2,4a,6a-trimethyl-1,2,3,4,4a,4b,5,6,6a, 7,8,9,10,10a, 10b,11-hexadecahydrochrysen-2-ol (H-7) and (2S,4aR,4bS,6aR,7R,10aS,10bS)-7-((2R,5R)-5-hydroxy-6-methylheptan-2-yl)-2,4a,6a-trimethyl-1,2,3,4,4a,4b,5,6,6a, 7,8,9,10,10a,10b,11-hexadecahydrochrysen-2-ol (H-8)

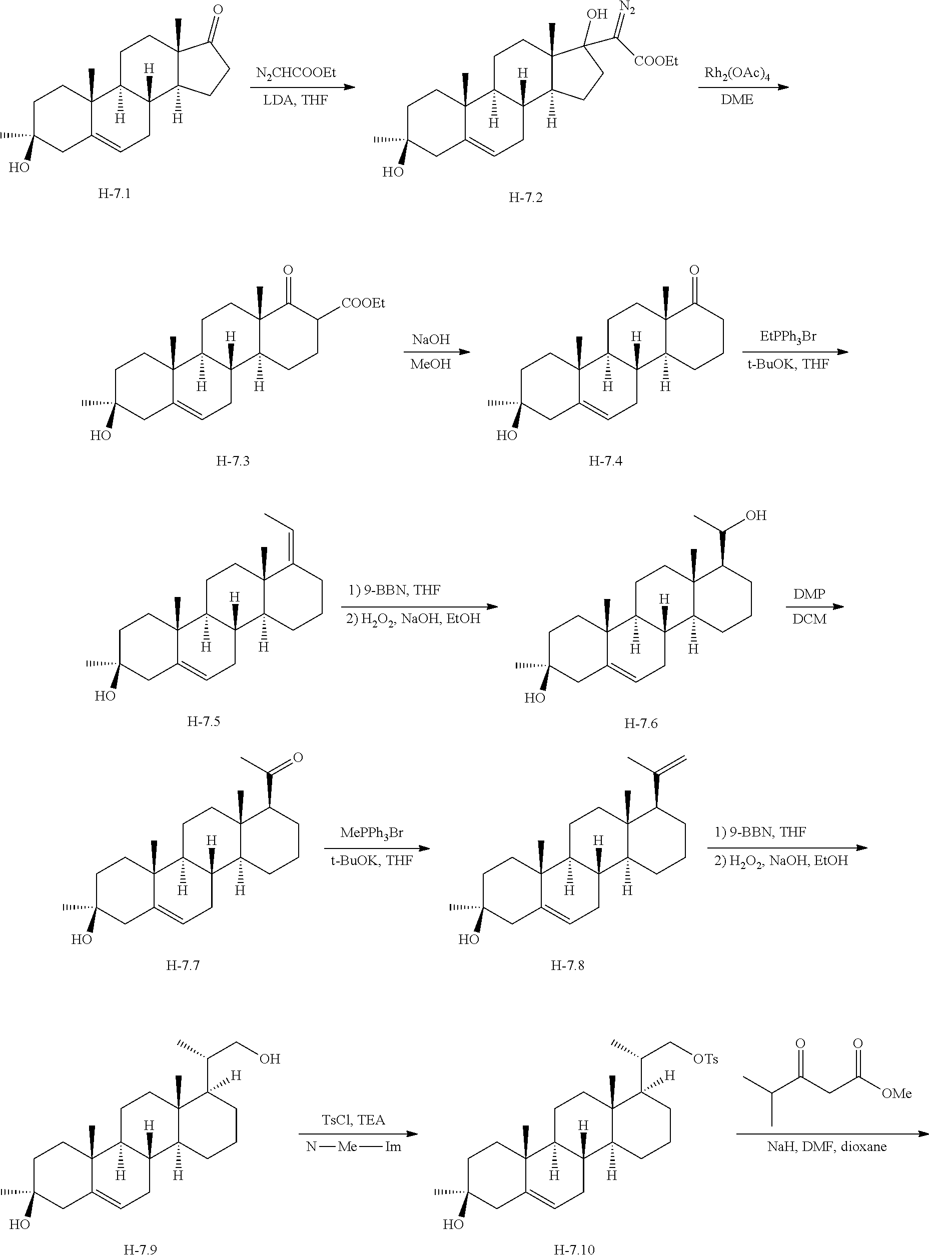

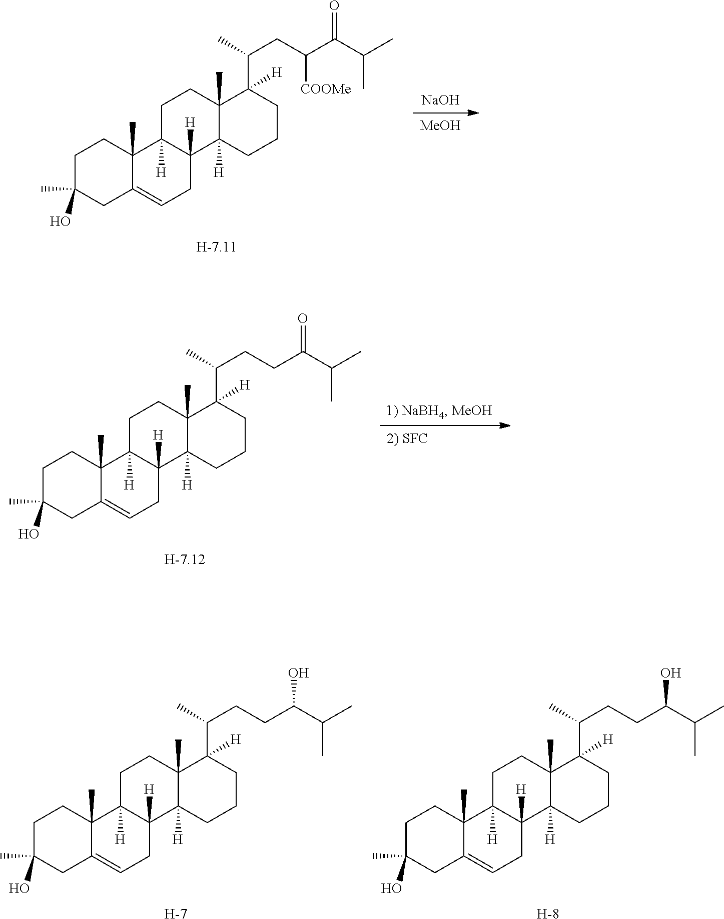

### Synthesis of H-7.2

To a solution of i-Pr₂NH (18.3 g, 181 mmol) in THF (75 mL) under N₂ was added n-BuLi (66.0 mL, 2.5 M, 165 mmol) at −60° C. and the resulting mixture was stirred at −60° C. for 10 min and 20° C. for 10 min. The LDA solution was added to a stirred solution of H-7.1 (10 g, 33.0 mmol; preparation described in US2016/022701A1) and ethyl diazoacetate (18.8 g, 17.3 mL, 165 mmol) in THF (250 mL) at −70° C. and stirred for 4 h. Acetic acid (9.90 g, 9.42 mL, 165 mmol) in THF (15 mL) was added, and the mixture was warmed to 25° C. and stirred for 16 h. The mixture was poured into H₂O (100 mL) and extracted with EtOAc (2×150 mL). The combined organic layers were washed with brine (2×100 mL), dried over anhydrous Na₂SO₄, filtered, and concentrated. The residue was purified by silica gel chromatography (0˜20% of EtOAc in PE) to give H-7.2 (9.8 g). ¹HNMR (400 MHz, CDCl₃) δ_{H} 5.30-5.27 (m, 1H), 4.31-4.19 (m, 2H), 2.44-2.35 (m, 1H), 2.23-2.11 (m, 1H), 1.94-1.83 (m, 3H), 1.79-1.64 (m, 4H), 1.51-1.30 (m, 12H), 1.28-1.21 (m, 3H), 1.10 (s, 3H), 1.02 (s, 3H), 0.94 (s, 3H).

### Synthesis of H-7.3

To a solution of H-7.2 (17 g, 40.8 mmol) in DME (200 mL) was added Rh₂(OAc)₄ (360 mg, 815 umol) and the resulting mixture was stirred at 25° C. for 16 h. The reaction mixture was concentrated to give H-7.3 (14.9 g).

### Synthesis of H-7.4

To a mixture of H-7.3 (14.9 g, 38.3 mmol) in MeOH (160 mL) was added H₂O (40 mL) and NaOH (15.3 g, 383 mmol) and the resulting mixture was stirred at 80° C. for 16 h. The reaction mixture was concentrated, and H₂O (50 mL) was added. The mixture was extracted with EtOAc (2×50 mL), and the combined organic layers were washed with brine (50 mL), dried over anhydrous Na₂SO₄, filtered, and concentrated to give H-7.4 (9 g). H-7.4 (10 mg, 0.032 mmol) was lyophilized to get H-7.4 (9.8 mg, 98.3%). ¹HNMR (400 MHz, CDCl₃) δ_{H} 5.34-5.27 (m, 1H), 2.72-2.60 (m, 1H), 2.45-2.37 (m, 1H), 2.25-1.96 (m, 4H), 1.85-1.61 (m, 6H), 1.56-1.14 (m, 8H), 1.114-1.04 (m, 7H), 1.01 (s, 3H), 0.99-0.93 (m, 1H). LC-ELSD/MS 30-90AB_2 min_E, purity>99%, MS ESI calcd. for C21H₃₁₀ [M−H₂O+H]⁺ 299.2, found 299.2.

### Synthesis of H-7.5

To a solution of EtPPh₃Br (31.2 g, 84.3 mmol) in THF (200 mL) was added t-BuOK (9.45 mg, 84.3 mmol) and the resulting mixture was stirred for 30 min at 45° C. A solution of H-7.4 (8.9 g, 28.1 mmol) in THF (50 mL) was added into the reaction at 45° C. and stirred for 12 h. The mixture was poured into saturated aqueous NH₄Cl (500 mL) and extracted with EtOAc (2×100 mL). The combined organic layers were washed with brine (100 mL), dried over anhydrous Na₂SO₄, filtered, and concentrated in vacuo. The mixture was purified by trituration with MeOH/H₂O (1:1, 500 mL) and separated by silica gel chromatography (0˜20% EtOAc in PE) to give H-7.5 (7.3 g, 79.1%). ¹HNMR (400 MHz, CDCl₃) δ_{H} 5.32-5.26 (m, 1H), 5.22-5.13 (m, 1H), 2.56-2.48 (m, 1H), 2.45-2.36 (m, 1H), 2.31-2.09 (m, 2H), 2.02-1.64 (m, 9H), 1.53-1.31 (m, 6H), 1.17-1.04 (m, 7H), 1.02-0.92 (m, 6H), 0.90-0.79 (m, 2H).

### Synthesis of H-7.6

To a solution of H-7.5 (7.3 g, 22.2 mmol) in THF (100 mL) was added 9-BBN (10.8 g, 44.4 mmol) and the resulting mixture was stirred at 25° C. for 16 h. To the mixture was added ethanol (15.6 mL, 266 mmol), followed by aqueous NaOH (53.2 mL, 5.0 M, 266 mmol). H₂O₂ (26.6 mL, 10 M, 266 mmol) was added and the mixture was stirred at 25° C. for 1 h. The mixture was quenched by 10% aqueous Na₂S₂O₃ (400 mL) and extracted with EtOAc (3×100 mL). The combined organic layers were washed with brine (200 mL), dried over anhydrous Na₂SO₄, filtered, concentrated and purified by silica gel chromatography (0˜40% EtOAc in PE) to give H-7.6 (6.8 g).

### Synthesis of H-7.7

To a solution of H-7.6 (6.3 g, 18.1 mmol) in DCM (100 mL) was added DMP (15.3 g, 36.2 mmol) at 25° C. and the resulting mixture was stirred for 30 min. To the mixture was added NaHCO₃ (200 mL, sat.) and Na₂S₂O₃ (200 mL, sat.) and the aqueous phase was extracted with DCM (2×200 mL). The combined organic layers were washed with NaHCO₃/Na₂S₂O₃ (1:1, 2×200 mL, sat.), brine (200 mL), dried over anhydrous Na₂SO₄, filtered, and concentrated. The residue was combined with another batch of H-7.7 (6.8 g) and was purified by silica gel chromatography (0-30% of EtOAc in PE) to give H-7.7 (6.4 g). ¹HNMR (400 MHz, CDCl₃) δ_{H} 5.29-5.26 (m, 1H), 2.43-2.24 (m, 2H), 2.13 (s, 3H), 2.00-1.95 (m, 1H), 1.87-1.54 (m, 11H), 1.46-1.16 (m, 7H), 1.10 (s, 3H), 1.05-1.00 (m, 1H), 0.97 (s, 3H), 0.93 (s, 3H), 0.86-0.76 (m, 1H).

### Synthesis of H-7.8

To a mixture of MePPh₃Br (32.5 g, 91 mmol) in THF (50 mL) was added t-BuOK (10.2 g, 91 mmol) at 25° C. under N₂ and the resulting mixture was stirred at 50° C. for 30 min. H-7.7 (6.3 g, 18.2 mmol) in THF (50 mL) was added in portions to keep the internal temperature below 55° C. After stirring at 55° C. for 16 h, the reaction mixture was quenched with 10% aqueous NH₄Cl (200 mL) at 15° C. The aqueous layer was extracted with EtOAc (3×50 mL) and the combined organic layers were washed with brine (100 mL), dried over anhydrous Na₂SO₄, filtered, and concentrated under vacuum. The product was triturated from MeOH/H₂O (1:1, 600 mL) to give H-7.8 (5.3 g). ¹HNMR (400 MHz, CDCl₃) δ_{H} 5.34-5.25 (m, 1H), 4.84-4.78 (m, 1H), 4.64-4.57 (m, 1H), 2.44-2.36 (m, 1H), 2.18-1.75 (m, 5H), 1.72 (s, 3H), 1.71-1.62 (m, 3H), 1.56-1.14 (m, 10H), 1.11 (s, 3H), 1.08-0.94 (m, 6H), 0.87-0.76 (m, 4H).

### Synthesis of H-7.9

To a solution of H-7.8 (5.3 g, 15.4 mmol) in THF (100 mL) was added 9-BBN dimer (7.51 g, 30.8 mmol) and the resulting mixture was stirred at 25° C. under N₂ for 16 h. To the mixture was added ethanol (10.8 mL, 184 mmol) and NaOH (36.8 mL, 5 M, 184 mmol). H₂O₂ (18.4 mL, 10 M, 184 mmol) was added dropwise at 25° C. and stirred for 1 h. To the mixture was added Na₂SO₃ (100 mL, 20% aq.) at 25° C. and the mixture was stirred for 10 min and filtered. The solid was washed with water (100 mL) to give H-7.9 (5.2 g). ¹HNMR (400 MHz, CDCl₃) δ_{H} 5.35-5.25 (m, 1H), 3.81-3.71 (m, 1H), 3.43-3.34 (m, 1H), 2.46-2.36 (m, 1H), 2.18-1.69 (m, 10H), 1.45-1.25 (m, 11H), 1.11 (s, 3H), 1.04-0.93 (m, 9H), 0.84-0.74 (m, 3H).

### Synthesis of H-7.10

To a solution of H-7.9 (5.2 g, 14.4 mmol) in DCM (60 mL) was added N-methylimidazole (2.36 g, 28.8 mmol), TEA (5.58 mL, 43.2 mmol) and TsCl (4.11 g, 21.6 mmol) and the resulting mixture was stirred at 25° C. for 4 h. The mixture was poured into NaHCO₃ (20 mL, saturated) and the aqueous phase was extracted with DCM (2×50 mL). The combined organic layers were washed with brine (50 mL), dried over anhydrous Na₂SO₄, filtered, and concentrated. The residue was purified by silica gel chromatography (0˜30% of EtOAc in PE) to give H-7.10 (3.7 g, 50%). ¹HNMR (400 MHz, CDCl₃) δ_{H} 7.78 (d, J=8.4 Hz, 2H), 7.34 (d, J=8.0 Hz, 2H), 5.30-5.26 (m, 1H), 4.16-4.11 (m, 1H), 3.65 (t, J=9.6 Hz, 1H), 2.51-2.34 (m, 5H), 2.13-1.92 (m, 3H), 1.84-1.59 (m, 6H), 1.49-1.11 (m, 10H), 1.10 (s, 3H), 0.97 (s, 3H), 0.94 (d, J=6.8 Hz, 3H), 0.91-0.79 (m, 3H), 0.69 (s, 3H).

### Synthesis of H-7.11

To a solution of NaH (918 mg, 60%, 23 mmol) in dixoane (10 mL) was added ethyl 3-oxobutanoate (4.28 g, 29.7 mmol) at 0° C. under N₂ and the resulting mixture was stirred for 1 h. DMF (15 mL) and H-7.10 (1.7 g, 3.3 mmol) in dixoane (20 mL) were added dropwise and stirred at 105° C. for 48 h. The aqueous phase was extracted with DCM (3×20 mL) and the combined organic layers were washed with water (2×50 mL), dried over anhydrous Na₂SO₄, filtered, and concentrated to give H-7.11 (10 g) which was used directly in the next step.

### Synthesis of H-7.12

To a solution of H-7.11 (10 g, 20.5 mmol) in MeOH (60 mL) was added H₂O (20 mL) and NaOH (4.07 g, 102 mmol) at 25° C. and the resulting mixture was stirred at 60° C. for 16 h. The reaction mixture was extracted with DCM (4×100 mL) and the combined organic layers were washed with brine (50 mL), dried over anhydrous Na₂SO₄, filtered, and concentrated. The residue was purified by silica gel chromatography (0-20% of EtOAc in PE) twice to give H-7.12 (150 mg). H-7.12 (10 mg) was triturated from MeCN (2 mL) to get H-7.12 (6.6 mg, 66.1%). ¹HNMR (400 MHz, CDCl₃) δ_{H} 5.33-5.26 (m, 1H), 2.64-2.54 (m, 1H), 2.50-2.30 (m, 3H), 2.17-2.08 (m, 1H), 2.01-1.60 (m, 8H), 1.53-1.32 (m, 7H), 1.32-0.99 (m, 13H), 0.98 (s, 3H), 0.97-0.88 (m, 4H), 0.88-0.84 (m, 6H), 0.76-0.69 (m, 1H). LC-ELSD/MS 30-90AB_2 min_E, purity>99%, MS ESI calcd. for C₂₉H₄₇₀ [M−H₂O+H]⁺ 411.4, found 411.4.

### Synthesis of H-7 and H-8

To a solution of H-7.12 (140 mg, 0.737 mmol) in MeOH (10 mL) was added NaBH₄ (100 mg, 2.63 mmol) at 25° C. under N₂ and the resulting mixture was stirred for 1 h. The reaction mixture was quenched with saturated aqueous NH₄Cl (20 mL) at 20° C. and the aqueous layer was extracted with EtOAc (2×20 mL). The combined organic layers were filtered and concentrated. The residue was purified by SFC (Column: DAICEL CHIRALPAK AD (250 mm*30 mm, 10 um); Condition: 0.1% NH₃H₂O IPA; Begin B: 35%; End B: 35%; Gradient Time (min): 70; FlowRate (ml/min): 70; Injections: 70) to give H-7 (48.6 mg, 32.6%) and H-8 (35.4 mg, 23.7%).

H-7: ¹HNMR (400 MHz, CDCl₃) δ_{H} 5.32-5.27 (m, 1H), 3.36-3.30 (m, 1H), 2.43-2.37 (m, 1H), 2.17-2.08 (m, 1H), 2.01-1.63 (m, 8H), 1.48-1.24 (m, 11H), 1.13-1.00 (m, 1H), 1.13-1.00 (m, 6H), 0.98 (s, 3H), 0.93-0.86 (m, 12H), 0.81 (s, 3H), 0.80-0.63 (m, 2H). LC-ELSD/MS 30-90AB_2 min_E, purity>99%, MS ESI calcd. for C₂₉H₄₉₀ [M−H₂O+H]⁺ 413.4, found 413.4.

H-8: ¹HNMR (400 MHz, CDCl₃) δ_{H} 5.31-5.27 (m, 1H), 3.34-3.27 (m, 1H), 2.44-2.38 (m, 1H), 2.17-2.07 (m, 1H), 2.01-1.89 (m, 2H), 1.84-1.59 (m, 7H), 1.55-1.12 (m, 14H), 1.11 (s, 3H), 0.98 (s, 3H), 0.94-0.86 (m, 12H), 0.83-0.69 (m, 5H). LC-ELSD/MS 30-90AB_2 min_E, purity>99%, MS ESI calcd. for C₂₉H₄₉₀ [M−H₂O+H]⁺ 413.4, found 413.4.

### Example 83: Synthesis of (2S,4aR,4bS,6aR,7R,10aS,10bS)-2-ethyl-4a,6a-dimethyl-7-((2R,5S)-6,6,6-trifluoro-5-hydroxy-5-methylhexan-2-yl)-1,2,3,4,4a,4b,5,6,6a,7,8,9,10,10a,10b,11-hexadecahydrochrysen-2-ol (H-9) and (2S,4aR,4bS,6aR,7R,10aS,10bS)-2-ethyl-4a,6a-dimethyl-7-((2R,5R)-6,6,6-trifluoro-5-hydroxy-5-methylhexan-2-yl)-1,2,3,4,4a,4b,5,6,6a,7,8,9,10,10a,10b,11-hexadecahydrochrysen-2-ol (H-10)

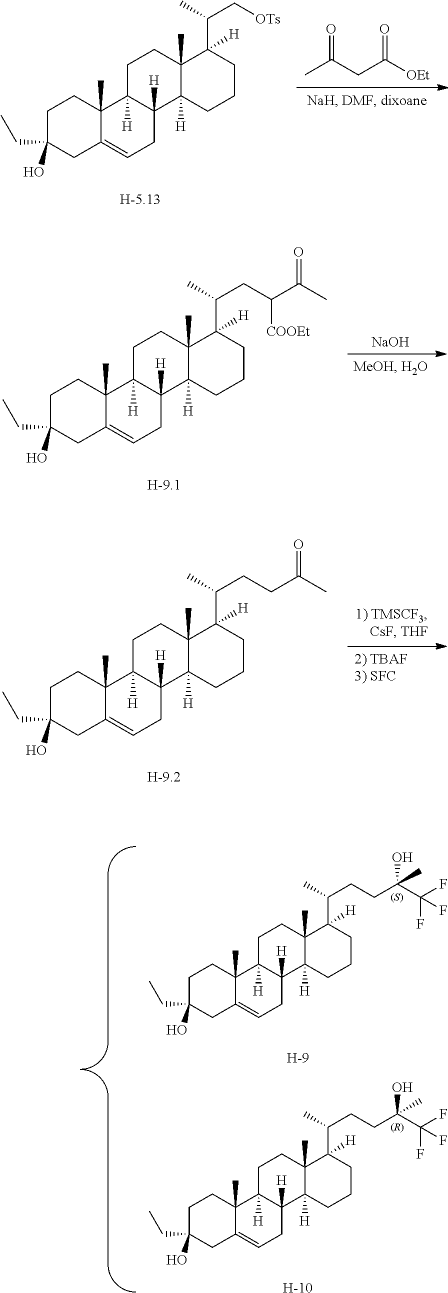

### Synthesis of H-9.1

To a solution of NaH (180 mg, 60%, 4.53 mmol) in dioxane (4 mL) was added ethyl 3-oxobutanoate (884 mg, 6.80 mmol) at 0° C. under N₂ and the resulting mixture was stirred for 1 h. DMF (2 mL) and H-5.13 (400 mg, 0.756 mmol) in dioxane (2 mL) were added dropwise and stirred at 105° C. for 16 h. The mixture was poured into saturated aqueous NH₄Cl (10 mL) and the aqueous phase was extracted with DCM (3×20 mL). The combined organic layers were washed with water (2×20 mL), dried over anhydrous Na₂SO₄, filtered, and concentrated to give H-9.1 (400 mg), which was used directly in the next step.

### Synthesis of H-9.2

To a mixture of H-9.1 (400 mg, 0.821 mmol) in MeOH (15 mL) was added H₂O (4 mL) and NaOH (163 mg, 4.10 mmol) at 25° C. and the resulting mixture was stirred at 20° C. for 16 h. The reaction mixture was extracted with DCM (4×20 mL) and the combined organic layers were washed with brine (30 mL), dried over anhydrous Na₂SO₄, filtered, and concentrated. The product was purified by silica gel chromatography (0-30% EtOAc in PE) and lyophilized to give H-9.2 (70 mg). ¹HNMR (400 MHz, CDCl₃) δ_{H} 5.29-5.26 (m, 1H), 2.53-2.41 (m, 1H), 2.39-2.28 (m, 2H), 2.18-2.07 (m, 4H), 2.06-2.01 (m, 1H), 1.94-1.59 (m, 8H), 1.53-1.30 (m, 9H), 1.22-1.04 (m, 3H), 1.00 (s, 3H), 0.98-0.89 (m, 4H), 0.88-0.83 (m, 9H), 0.76-0.69 (m, 1H). LC-ELSD/MS 30-90AB_2 min_E, purity>99%, MS ESI calcd. for C28H₄₅₀ [M−H₂O+H]⁺ 397.4, found 397.4.

### Synthesis of H-9 and H-10

To a solution of H-9.2 (60 mg, 0.1446 mmol) and CsF (218 mg, 1.44 mmol) in THF (10 mL) was added TMSCF₃ (307 mg, 2.16 mmol) dropwise at 0° C. under N₂. After stirring at 20° C. for 2 h, TBAF (2.89 mL, 2.89 mmol, 1M TBAF in THF) was added and the mixture was stirred at 20° C. for 16 h. The mixture was poured into water (20 mL) and extracted with EtOAc (2×20 mL). The combined organic layers were washed with brine (2×20 mL), dried over anhydrous Na₂SO₄, filtered, and concentrated. The residue was purified by silica gel chromatography (0˜60% EtOAc in PE) and separated by SFC (Column: DAICEL CHIRALPAK IG (250 mm*30 mm, 10 um); Condition: 0.1% NH₃H₂O MEOH; Begin B: 40%; End B: 40%; FlowRate (ml/min): 80; Injections: 40) to give H-9 (17.6 mg, 25.2%) and H-10 (13.1 mg, 18.8%).

H-9: ¹HNMR (400 MHz, CDCl₃) δ_{H} 5.32-5.24 (m, 1H), 2.38-2.30 (m, 1H), 2.17-2.07 (m, 1H), 2.06-2.00 (m, 1H), 1.95-1.87 (m, 1H), 1.85-1.58 (m, 9H), 1.53-1.34 (m, 9H), 1.31 (s, 3H), 1.26-1.04 (m, 3H), 1.00 (s, 3H), 0.99-0.87 (m, 8H), 0.85 (t, J=7.2 Hz, 3H), 0.81 (s, 3H), 0.78-0.71 (m, 1H). ¹⁹F NMR (376 MHz, CDCl₃) δ_{F} −83.09 (s). LC-ELSD/MS 30-90AB_2 min_E, purity>99%, MS ESI calcd. for C₂₉H₄₆F₃O [M−H₂O+H]⁺ 467.4, found 467.4.

H-10: ¹HNMR (400 MHz, CDCl₃) δ_{H} 5.32-5.24 (m, 1H), 2.39-2.29 (m, 1H), 2.17-2.08 (m, 1H), 2.06-2.01 (m, 1H), 1.94-1.57 (m, 10H), 1.53-1.34 (m, 9H), 1.32 (s, 3H), 1.30-1.04 (m, 4H), 1.00 (s, 3H), 0.98-0.89 (m, 7H), 0.85 (t, J=7.6 Hz, 3H), 0.81 (s, 3H), 0.78-0.71 (m, 1H). ¹⁹F NMR (376 MHz, CDCl₃) δ_{F} −82.87 (s). LC-ELSD/MS 30-90AB_2 min_E, purity>99%, MS ESI calcd. for C₂₉H₄₆F₃O [M−H₂O+H]⁺ 467.4, found 467.4.

### Example 84: Synthesis of (2S,4aR,4bS,6aR,7R,10aS,10bS)-2,4a,6a-trimethyl-7-((2R,5R)-6,6,6-trifluoro-5-hydroxy-5-methylhexan-2-yl)-1,2,3,4,4a,4b,5,6,6a,7,8,9,10,10a,10b,11-hexadecahydrochrysen-2-ol (H-11)

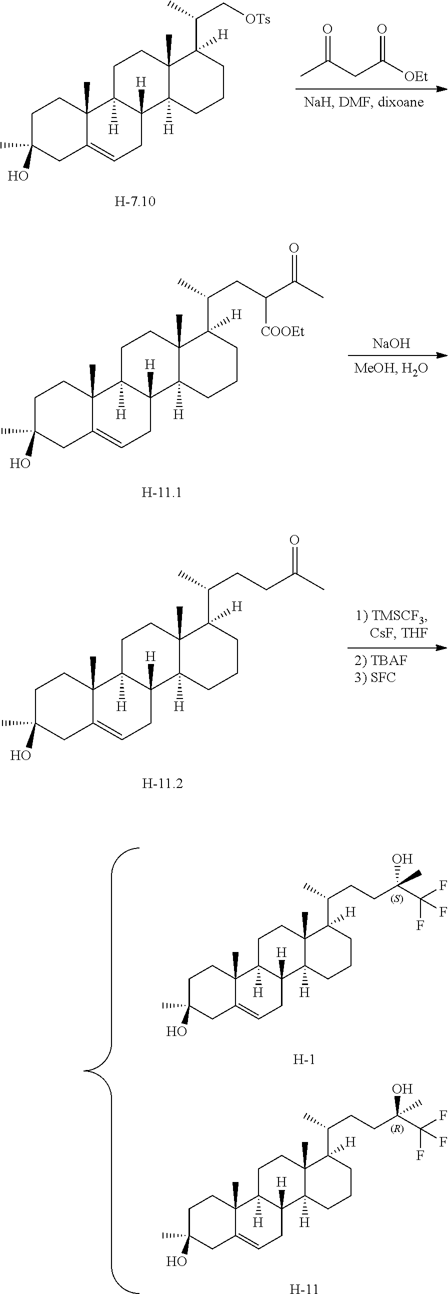

### Synthesis of H-11.1

To a solution of NaH (538 mg, 60%, 13.5 mmol) in dioxane (10 mL) was added ethyl 3-oxobutanoate (2.26 g, 17.4 mmol) at 0° C. under N₂ and the resulting mixture was stirred for 1 h. DMF (15 mL) and H-7.10 (1 g, 1.94 mmol) in dioxane (10 mL) were added dropwise and stirred at 105° C. for 48 h. The mixture was poured into saturated aqueous NH₄Cl (30 mL), and the aqueous phase was extracted with DCM (3×20 mL). The combined organic layers were washed with water (2×50 mL), dried over anhydrous Na₂SO₄, filtered, and concentrated to give H-11.1 (5 g), which was used directly in the next step.

### Synthesis of H-11.2

To a solution of H-11.1 (5 g, 10.5 mmol) in MeOH (30 mL) was added H₂O (10 mL) and NaOH (2.09 g, 52.5 mmol) at 25° C. and the resulting mixture was stirred at 60° C. for 16 h. The reaction mixture was extracted with DCM (2×50 mL) and the combined organic layers were washed with brine (50 mL), dried over anhydrous Na₂SO₄, filtered, and concentrated. The residue was purified by silica gel chromatography (0-30% of EtOAc in PE) to give H-11.2 (150 mg, 3.57%). The product was combined with another batch of H-11.2 (220 mg) and was triturated from MeCN (2 mL) to give H-11.2 (220 mg, 100%). ¹HNMR (400 MHz, CDCl₃) δ_{H} 5.35-5.26 (m, 1H), 2.53-2.27 (m, 3H), 2.17-2.07 (m, 4H), 2.02-1.95 (m, 1H), 1.93-1.87 (m, 1H), 1.83-1.66 (m, 5H), 1.56-1.34 (m, 8H), 1.33-1.05 (m, 7H), 0.98 (s, 3H), 0.97-0.88 (m, 4H), 0.86 (d, J=6.8 Hz, 3H), 0.83 (s, 3H), 0.77-0.69 (m, 1H). LC-ELSD/MS 30-90AB_2 min_E, purity>99%, MS ESI calcd. for C₂₇H₄₃₀ [M−H₂O+H]⁺ 383.3, found 383.3.

### Synthesis of H-11

To a solution of H-11.2 (190 mg, 0.47 mmol) and CsF (144 mg, 0.948 mmol) in THF (10 mL) was added TMSCF₃ (201 mg, 1.42 mmol) at 0° C. under N₂. After stirring at 20° C. for 3 h, TBAF (1.89 mL, 1.89 mmol, 1M TBAF in THF) was added. After stirring at 20° C. for 16 h, the mixture was poured into water (20 mL) and extracted with EtOAc (2×20 mL). The combined organic layers were washed with brine (2×20 mL), dried over anhydrous Na₂SO₄, filtered, and concentrated. The residue was purified by silica gel chromatography (0˜60% EtOAc in PE) and separated by SFC (Column: DAICEL CHIRALPAK AD (250 mm*30 mm, 10 um); Condition: 0.1% NH₃H₂O IPA; Begin B: 25%; End B: 25%; FlowRate (mL/min): 60; Injections: 60) twice to give H-1 (28.3 mg, 12.7%) and H-11 (22.1 mg, 9.9%).

H-11: ¹HNMR (400 MHz, CDCl₃) δ_{H} 5.33-5.26 (m, 1H), 2.45-2.35 (m, 1H), 2.18-2.08 (m, 1H), 2.02-1.95 (m, 1H), 1.94-1.87 (m, 1H), 1.84-1.57 (m, 8H), 1.54-1.31 (m, 11H), 1.30-1.02 (m, 6H), 1.01-0.86 (m, 11H), 0.81 (s, 3H), 0.79-0.72 (m, 1H). ¹⁹F NMR (376 MHz, CDCl₃) δ_{F} −82.87 (s). LC-ELSD/MS 30-90AB_2 min_E, purity>99%, MS ESI calcd. for C28H+F₃O [M−H₂O+H]⁺ 453.4, found 453.4.

### Example 85: Assay Methods and Biological Data

Compounds of the present disclosure can be evaluated using various in vitro and in vivo assays described in the literature; examples of which are described below.

The following examples are offered to illustrate the biological activity of the compounds, pharmaceutical compositions, and methods provided herein and are not to be construed in any way as limiting the scope thereof.

### NMDA Modulation

### Whole-Cell Patch Clamp of Mammalian Cells (Syncropatch 384i (Nanion Technologies))

Whole-cell patch clamp electrophysiology was used to investigate the effects of compounds on GluN1/GluN2A N-methyl-D-aspartate (NMDA) glutamate receptors expressed in mammalian cells.

### Cell Culture

Human embryonic kidney (HEK293) cells expressing the recombinant human GluN1/GluN2A receptor under the control of a tetracycline-inducible expression system were used. Cells were maintained in Dulbecco's Modified Eagle's Medium (DMEM) with 1% glutamine, 10% Tetracycline System Approved fetal bovine serum (FBS), and NMDA inhibitors. Cells were cultured in a humidified 5% CO₂ incubator at 37° C. Cells were induced with 50 ng/ml tetracycline 18-24 hours prior to testing. A cocktail of channel blockers was added to the media to prevent cell death.

Cells were 60-80% confluent at the time of harvest. Cells were detached from the flask TrypLE Enzyme Express (Thermo Fisher) at 37° C., and then cold extracellular solution was added to the flask, followed by incubation at 4° C. and then trituration of the cells. The cell solution was then placed on a shaker on the Syncropatch platform at 10° C. with a shake speed of a minimum 200 rpm for at least 30 minutes prior to testing.

### Solutions

Extracellular (EC) solution (in mM) —140 NaCl, 4 KCl, 5 CaCl2, 10 HEPES, 5 Glucose, pH 7.4 with NaOH. All external solutions contained 0.2% DMSO and 0.01% Kolliphor® EL (C₅₁₃₅, Sigma) throughout the experiment.

Intracellular (IC) solution (in mM) —120 CsF, 10 mM EGTA, 10 NaCl, 10 HEPES, 4 NaATP, 2 MgCl2, pH 7.2 with CsOH.

### Plate Preparation

First, the DMSO Master Plate was prepared, in which the DMSO stock of each test compound was serially diluted in 100% DMSO to 1000× the final assay concentration. Immediately prior to assay testing, the compounds were diluted 1:100 by transferring solutions from the DMSO Mast Plate to the Intermediate Plate pre-filled with EC solution using a Biomek FX automated liquid handling system. Compounds were then diluted further with extracellular solution into two pre-incubation plates—1) compound from the Intermediate Plate was mixed with EC solution at a 1:5 dilution to create a 2× Compound Pre-Incubation Plate which was diluted a further 1:2 when added to the Syncropatch for the initial compound pre-incubation. And 2) compound from the Intermediate plate was mixed with EC solution at a 1:10 Dilution to create the 1× Compound Pre-Incubation Plate, which was used for subsequent compound pre-incubations. Finally, a Compound+Agonist Plate was prepared by mixing compound from the 2× Compound Pre-Incubation Plate with 2× concentration of co-agonists in a 1:1 ratio.

### Voltage Protocol

Each experiment sequentially included cell catching, sealing, whole-cell formation, liquid application, recording and data acquisition. Whole-cell patch clamp recordings were performed using multi-hole high resistance chips on the SyncroPatch 384i (Nanion Technologies). A steady-state voltage pulse at -80 mV was applied throughout the assay and a 10 second recording window was triggered around 1 second prior to application of co-agonists. Currents were leak corrected and sampled at 5 kHz.

### Application Protocol

A fast application was used, in which 5 μl co-agonist was rapidly applied at a speed of 30 μl/s and then rapidly removed from the well. To test for modulator activity, the co-agonists glutamate (1 μM) and glycine (100 μM) were applied three times to show activation reproducibility, followed by 120 seconds pre-incubation of test compound alone. The test compound was then re-applied in the presence of co-agonists two times. This was followed by application of the agonist alone, although test compound may not be fully washed from the well prior to this step. Finally, a saturating concentration of co-agonists (glutamate 100 μM/glycine 100 μM) was applied.

### Analysis

The voltage protocol generation data collection and analysis were performed on PatchControll384 and DataControll384. For each concentration tested, the fold peak amplitude increase was generated using the following equation: (Icomp/Icontrol)-1, where Icomp is the mean current peak amplitude in the presence of the test compound and co-agonists from the two compound/co-agonist applications, and Icontrol is the mean current peak amplitude in the presence of agonist alone from the second and third co-agonist applications. Test compounds were evaluated at 8 concentrations, with at least 2 replicate wells per concentration. The effect of all concentrations was then fitted with a four-parameter logistic curve fit using least squares regression and the EC50 and Emax were calculated. (GraphPad Prism). For compounds that were tested on multiple assays, the geometric mean of the EC50 and arithmetic mean of the Emax was calculated using the parameters derived from the curve fits on each individual assay.

Experiments were conducted as described above and the results are shown in Tables A-2, B-2, C-2, D-2, E-2, F-2, G-2, and H-2 below.

TABLE A-2

| Cmpd | Structure | EC50 (nM) | Emax (%) |
|---|---|---|---|
| A-1 | 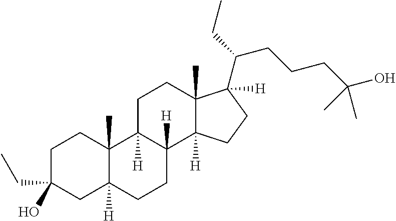 | A | F |
| A-2 | 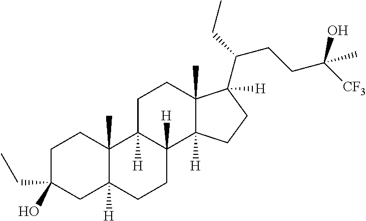 | C | F |
| A-3 | 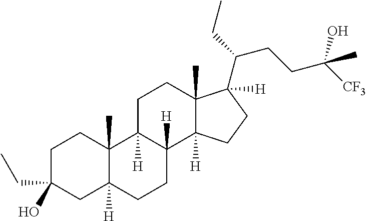 | A | G |
| A-4 | 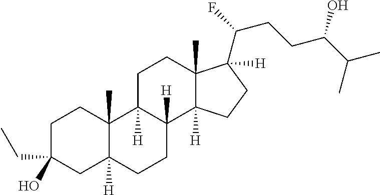 | A | G |
| A-5 | 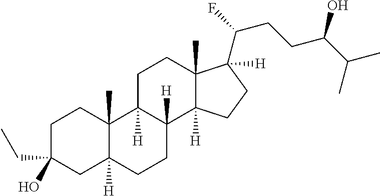 | A | G |
| A-6 | 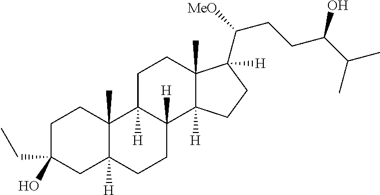 | B | G |
| A-7 | 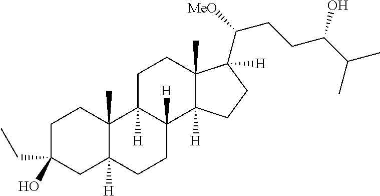 | B | G |
| A-8 | 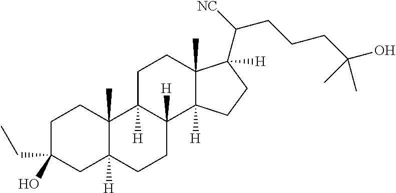 | A | F |
| A-9 | 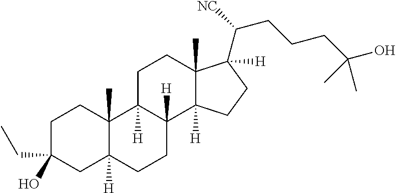 | A | G |
| A-9a | 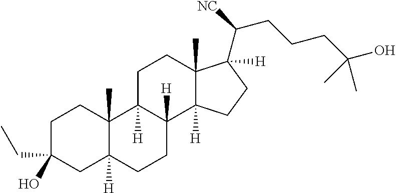 | A | E |
| A-10 | 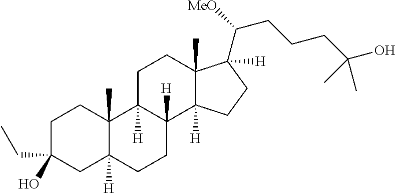 | B | F |
| A-11 | 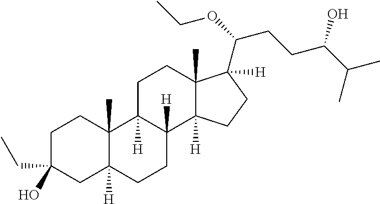 | D | N/A |
| A-11a | 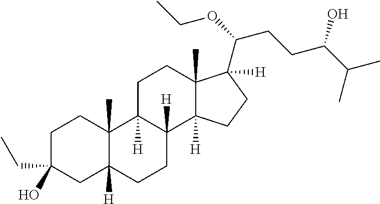 | D | E |
| A-12 | 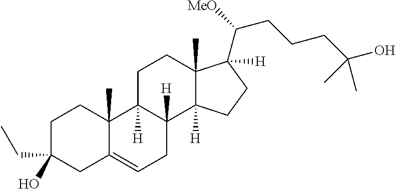 | A | F |
| A-13 | 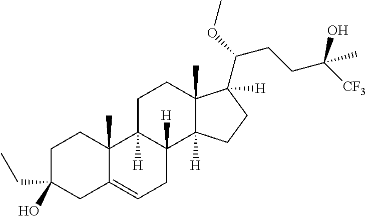 | C | G |
| A-14 | 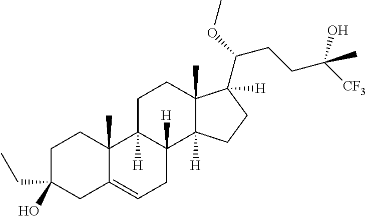 | A | G |
| A-15 | 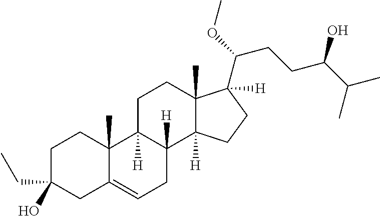 | A | G |
| A-16 | 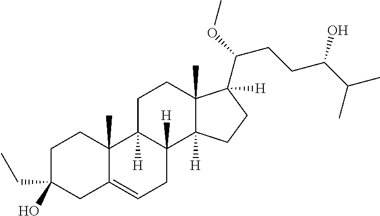 | A | G |
| A-17 | 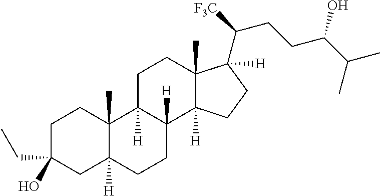 | D | N/A |
| A-18 | 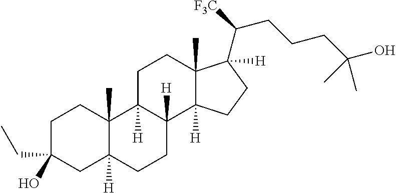 | D | N/A |
| A-19 | 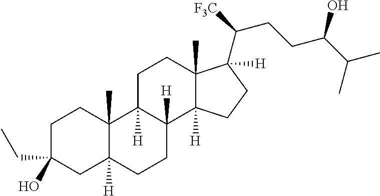 | D | N/A |
| A-20 | 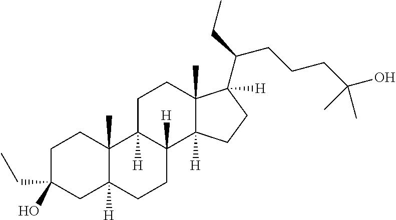 | D | N/A |
| A-23 | 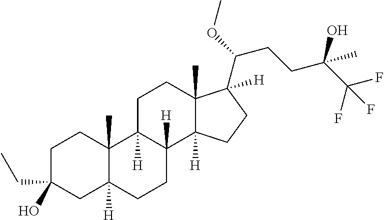 | C | N/A |
| A-24 | 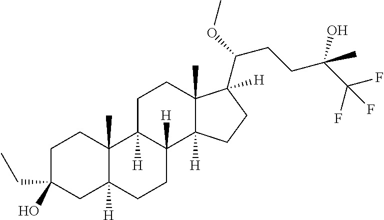 | B | N/A |

For Table A-2,
“A” indicates an EC50 of 1 to 100 nM,
“B” indicates an EC50 of greater than 100 nM up to 500 nM,
“C” indicates an EC50 greater than 500 nM up to 1 μM;
“D” indicates an EC50 of greater than 1 μM,
“E” indicates an Emax of up to 100%,
“F” indicates an Emax between 100% and 200%, and
“G” indicates an Emax greater than 200%.

TABLE B-2

| Cmpd | Structure | EC50 (nM) | Emax (%) |
|---|---|---|---|
| B-1 | 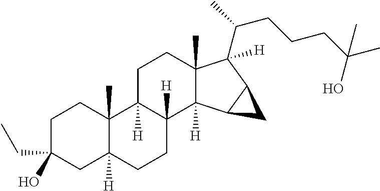 | A | H |
| B-2 | 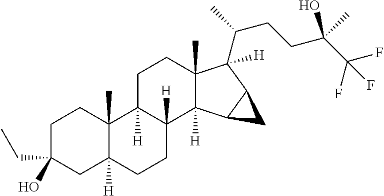 | B | H |
| B-3 | 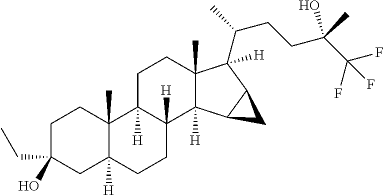 | A | H |
| B-4 | 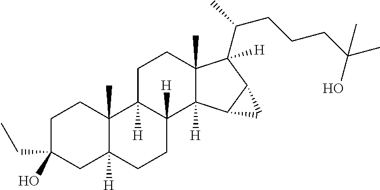 | A | H |
| B-5 | 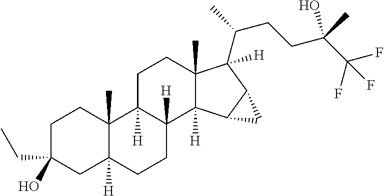 | B | H |
| B-6 | 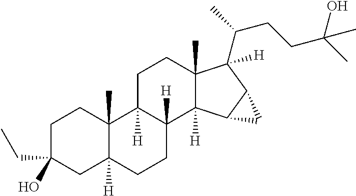 | B | H |
| B-7 | 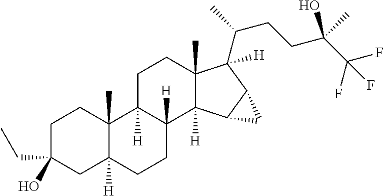 | B | G |
| B-8 | 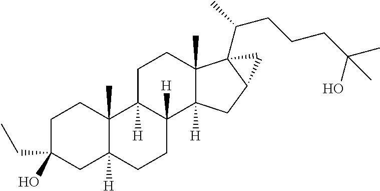 | D | G |
| B-10 | 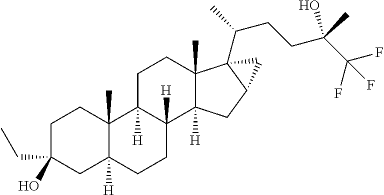 | A | G |
| B-11 | 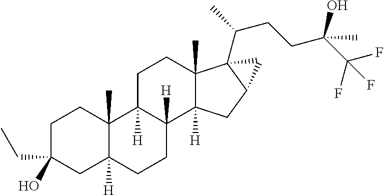 | A | G |
| B-12 | 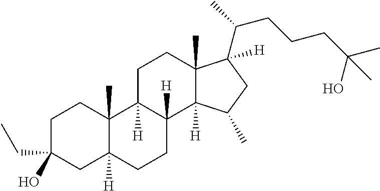 | A | G |
| B-13 | 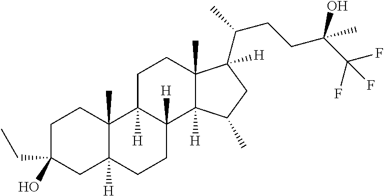 | B | H |
| B-14 | 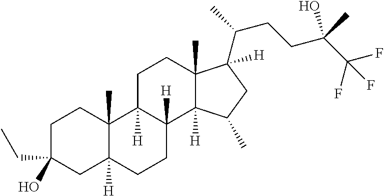 | A | H |
| B-15 | 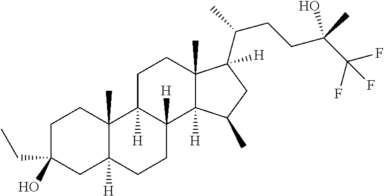 | A | G |
| B-16 | 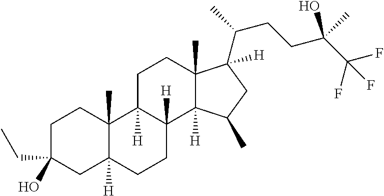 | B | G |
| B-17 | 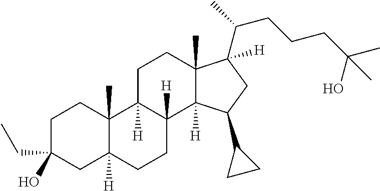 | B | G |
| B-18 | 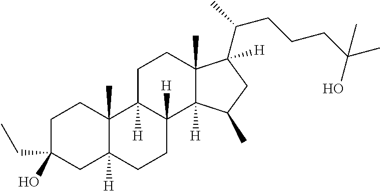 | A | G |
| B-19 | 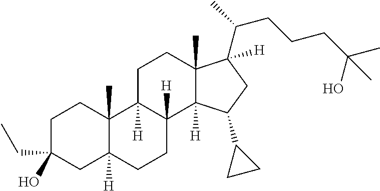 | B | G |
| B-20 | 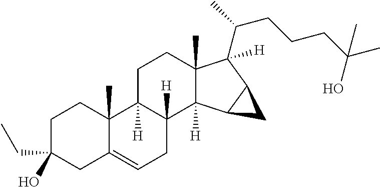 | A | G |
| B-21 | 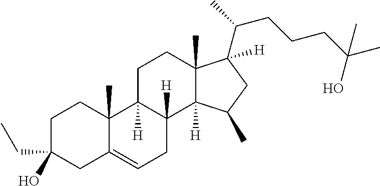 | A | N/A |
| B-22 | 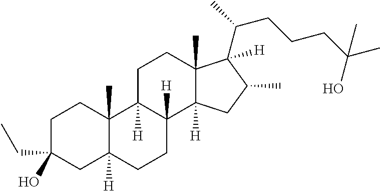 | B | H |
| B-23 | 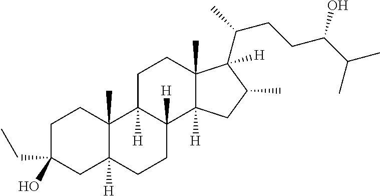 | A | H |
| B-24 | 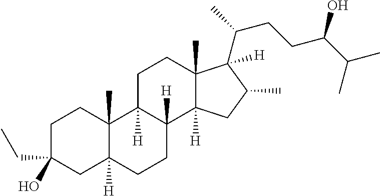 | B | H |

For Table B-2,
“A” indicates an EC50 of 1 to 100 nM,
“B” indicates an EC50 of greater than 100 nM up to 500 nM,
“C” indicates an EC50 greater than 500 nM up to 1 μM;
“D” indicates an EC50 of greater than 1 μM,
“F” indicates an Emax of up to 100%, and
“G” indicates an Emax between 100% and 200%, and
“H” indicates an Emax greater than 200%.

TABLE C-2

| Cmpd | Structure | EC50 (nM) | Emax (%) |
|---|---|---|---|
| C-1 | 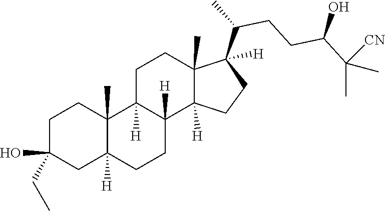 | A | H |
| C-2 | 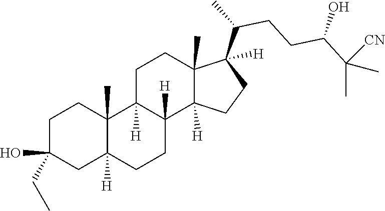 | A | G |
| C-3 | 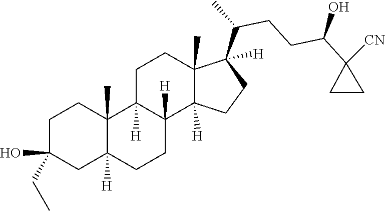 | A | H |
| C-4 | 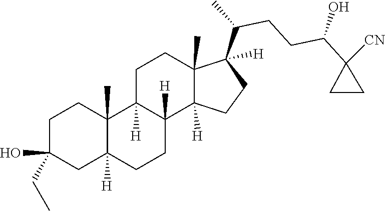 | B | F |
| C-5 | 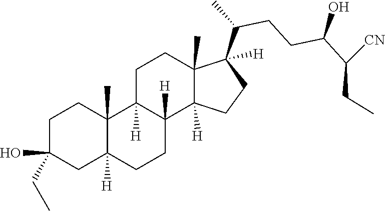 | A | H |
| C-6 | 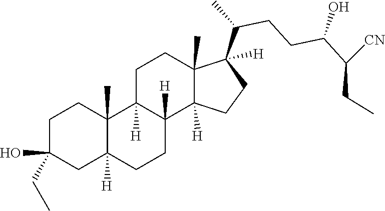 | B | G |
| C-7 | 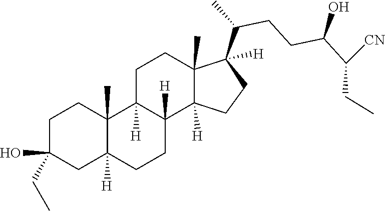 | A | H |
| C-8 | 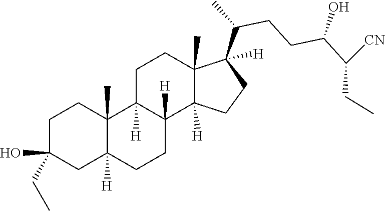 | A | F |
| C-9 | 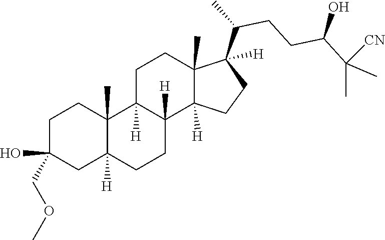 | A | F |
| C-10 | 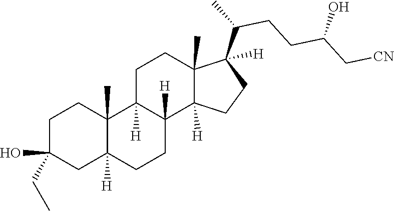 | B | F |
| C-11 | 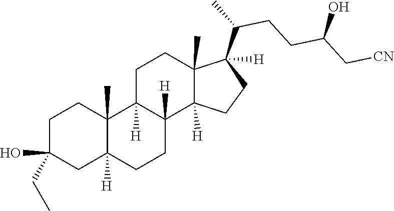 | A | G |
| C-12 | 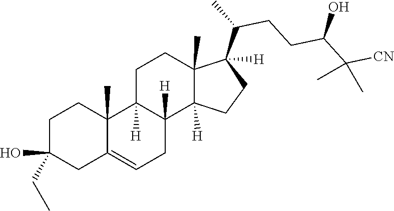 | A | H |
| C-13 | 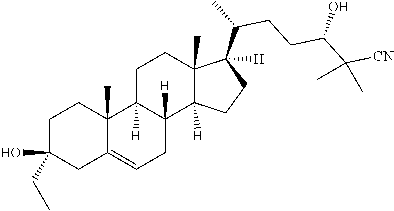 | A | G |
| C-18 | 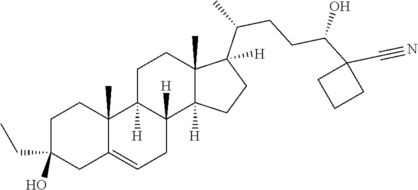 | B | N/A |
| C-19 | 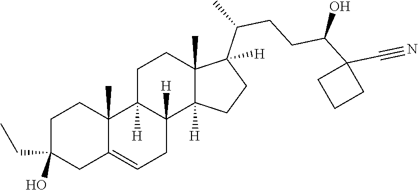 | A | N/A |
| C-20 | 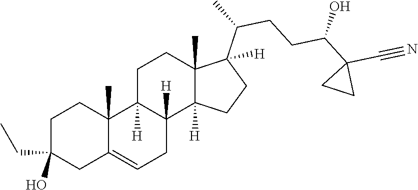 | B | N/A |
| C-21 | 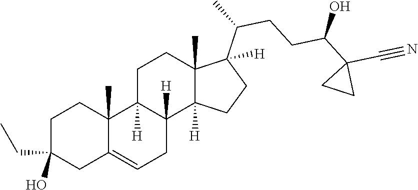 | A | N/A |
| C-22 | 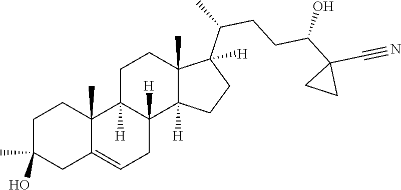 | A | F |
| C-23 | 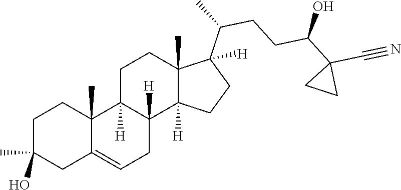 | A | N/A |
| C-24 | 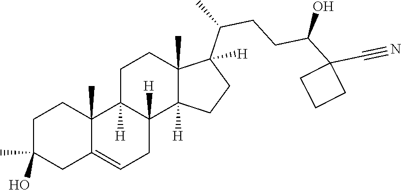 | A | N/A |
| C-25 | 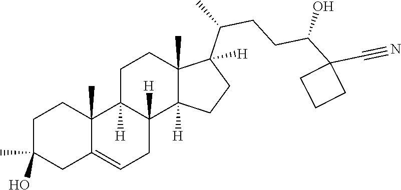 | A | F |
| C-26 | 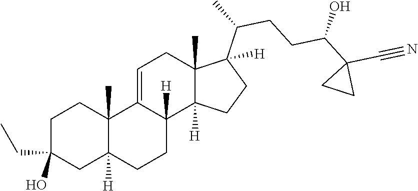 | A | N/A |
| C-27 | 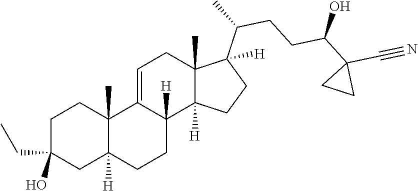 | A | N/A |
| C-28 | 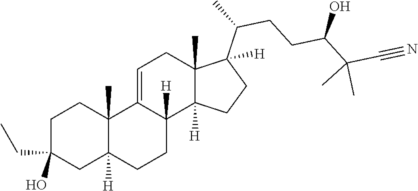 | A | N/A |
| C-29 | 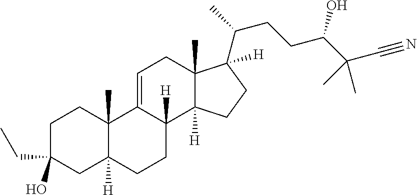 | A | N/A |
| C-30 | 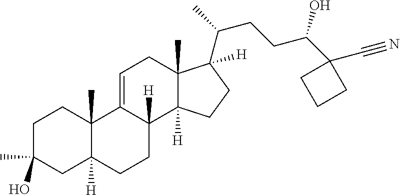 | B | N/A |
| C-31 | 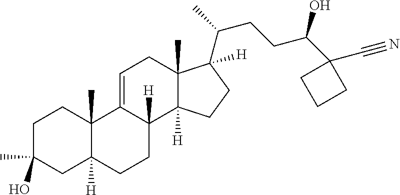 | A | N/A |
| C-32 | 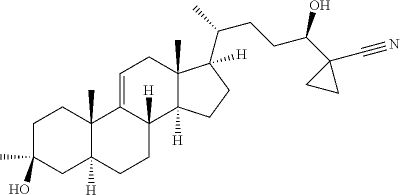 | A | N/A |
| C-33 | 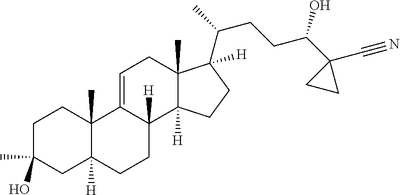 | B | N/A |
| C-34 | 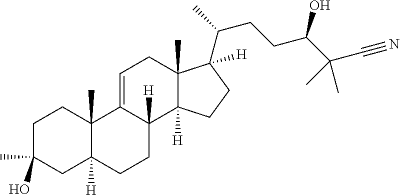 | A | N/A |
| C-35 | 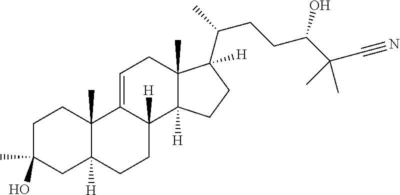 | A | N/A |

For Table C-2, “A” indicates an EC50 of 1 to 100 nM, “B” indicates an EC50 of greater than 100 nM up to 500 nM, “C” indicates an EC50 greater than 500 nM up to 1 μM; “D” indicates an EC50 of greater than 1 μM, “F” indicates an Emax of up to 100%, and “G” indicates an Emax between 100% and 200%, and “H” indicates an Emax greater than 200%.

TABLE D-2

| Cmpd | Structure | EC50 (nM) | Emax |
|---|---|---|---|
| D-1 | 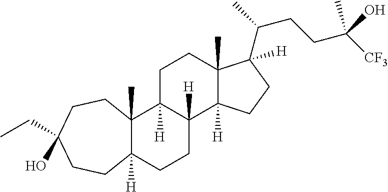 | D | F |
| D-2 | 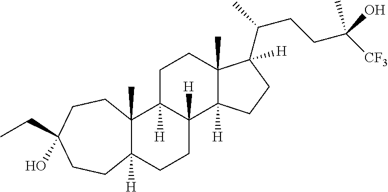 | D | F |
| D-3 | 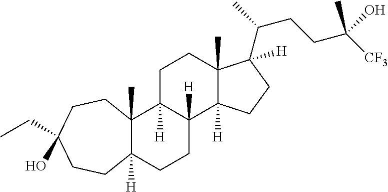 | B | G |
| D-4 | 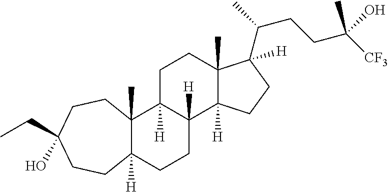 | D | N/A |
| D-5 | 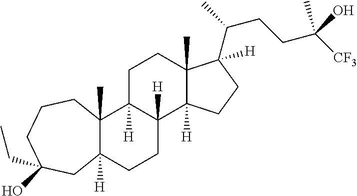 | B | F |
| D-6 | 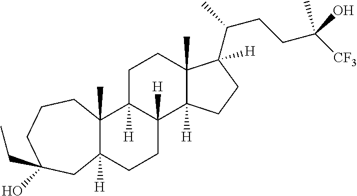 | A | G |
| D-7 | 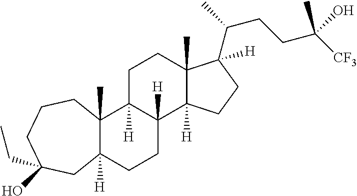 | A | G |
| D-8 | 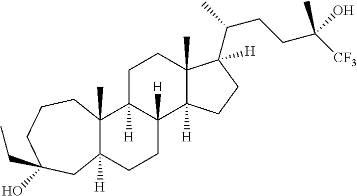 | D | G |
| D-9 | 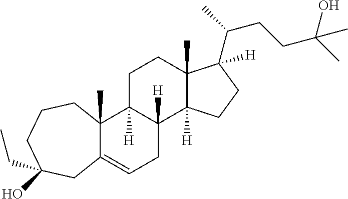 | B | G |
| D-10 | 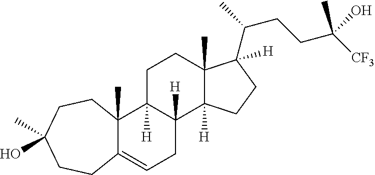 | B | H |
| D-11 | 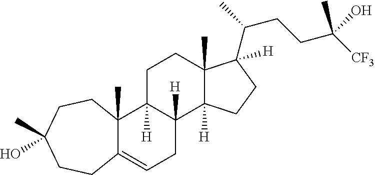 | D | H |
| D-12 | 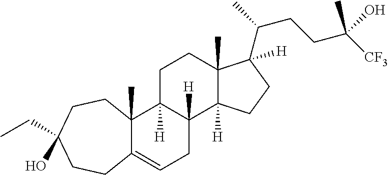 | B | H |
| D-13 | 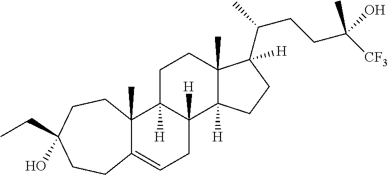 | D | H |
| D-14 | 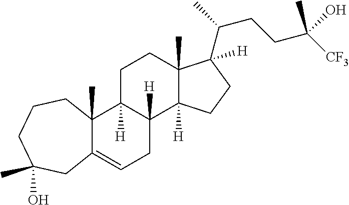 | D | N/A |
| D-15 | 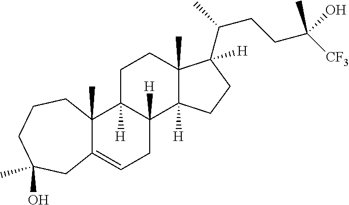 | B | F |
| D-16 | 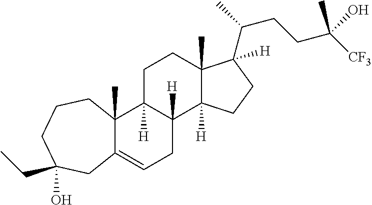 | D | F |
| D-17 | 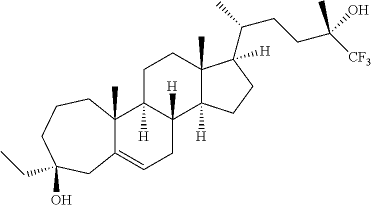 | B | G |

For Table D-2, “A” indicates an EC50 of 1 to 100 nM, “B” indicates an EC50 of greater than 100 nM up to 500 nM, “C” indicates an EC50 greater than 500 nM up to 1 μM; “D” indicates an EC50 of greater than 1 μM; “F” indicates an Emax of up to 100%; “G” indicates an Emax between 100% and 200%; and “H” indicates an Emax greater than 200%.

TABLE E-2

| Cmpd | Structure | EC50 (nM) | Emax (%) |
|---|---|---|---|
| E-1 | 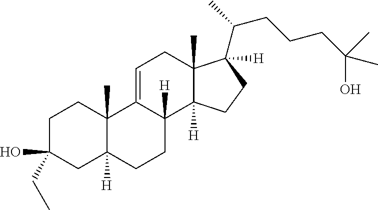 | A | F |
| E-2 | 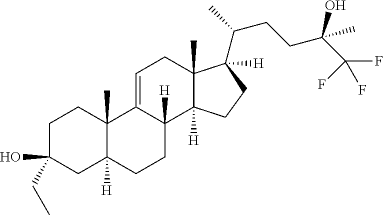 | B | H |
| E-3 | 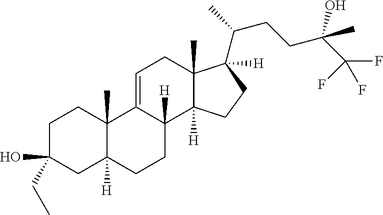 | A | H |
| E-4 | 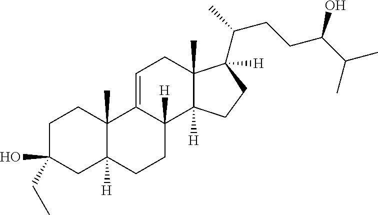 | A | H |
| E-5 | 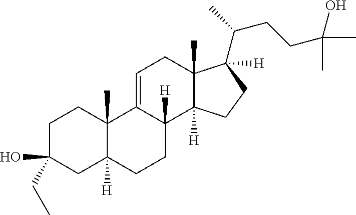 | A | G |
| E-6 | 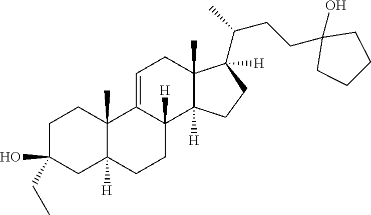 | A | H |
| E-7 | 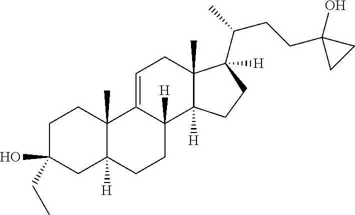 | B | H |
| E-8 | 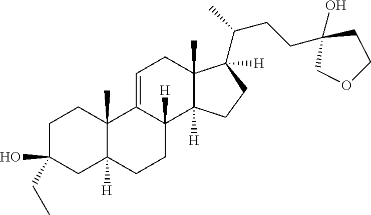 | A | H |
| E-9 | 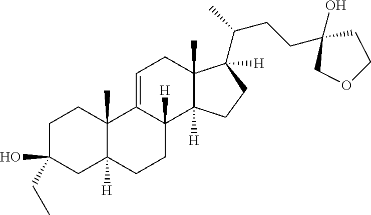 | A | H |
| E-10 | 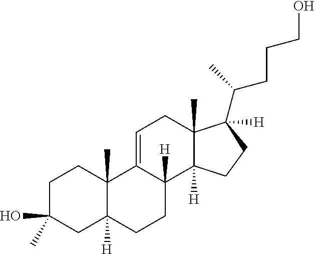 | A | F |
| E-11 | 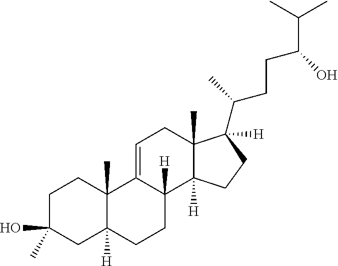 | A | G |
| E-12 | 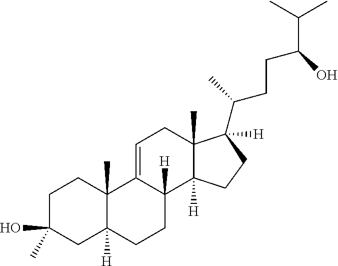 | A | G |
| E-13 | 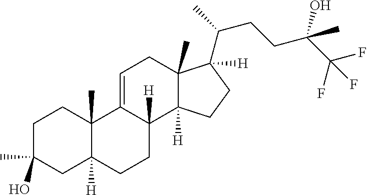 | A | N/A |
| E-14 | 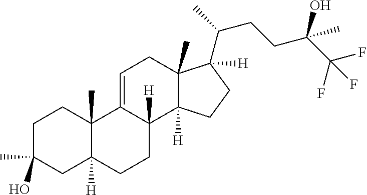 | B | N/A |
| E-15 | 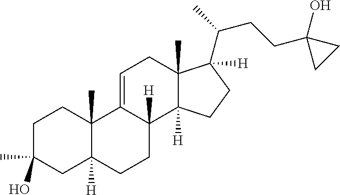 | A | N/A |
| E-16 | 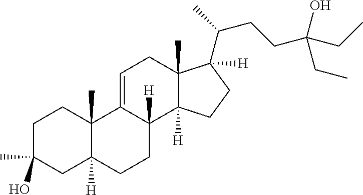 | C | N/A |
| C-26 | 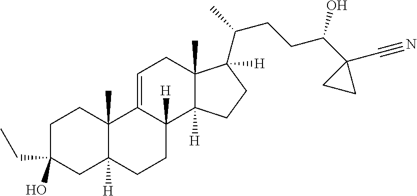 | A | N/A |
| C-27 | 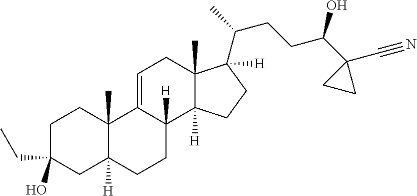 | A | N/A |
| C-28 | 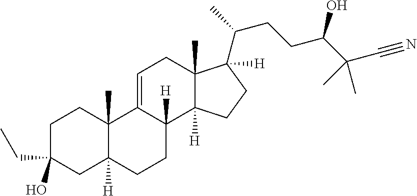 | A | N/A |
| C-29 | 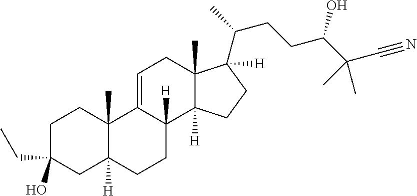 | A | N/A |
| C-30 | 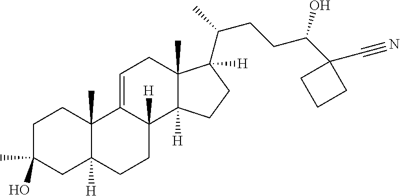 | B | N/A |
| C-31 | 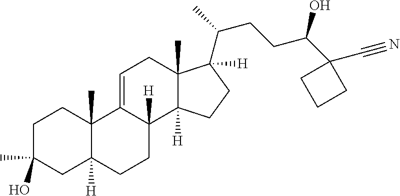 | A | N/A |
| C-32 | 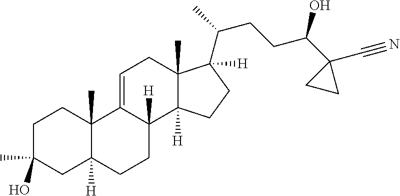 | A | N/A |
| C-33 | 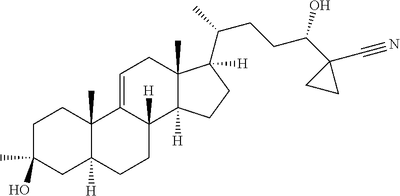 | B | N/A |
| C-34 | 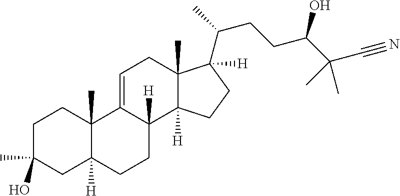 | A | N/A |
| C-35 | 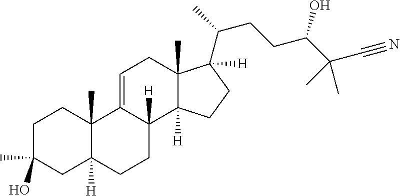 | A | N/A |

For Table E-2, “A” indicates an EC50 of 1 to 100 nM, “B” indicates an EC50 of greater than 100 nM up to 500 nM, “C” indicates an EC50 greater than 500 nM up to 1 μM; “D” indicates an EC50 of greater than 1 μM, “F” indicates an Emax of up to 100%, and “G” indicates an Emax between 100% and 200%, and “H” indicates an Emax greater than 200%.

TABLE F-2

| Cmpd | Structure | EC50 (nM) | Emax |
|---|---|---|---|
| F-3 | 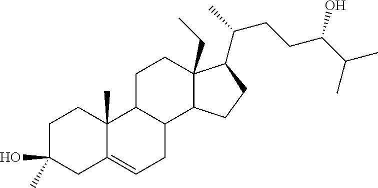 | B | N/A |
| F-4 | 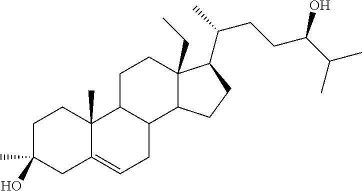 | B | N/A |
| F-5 | 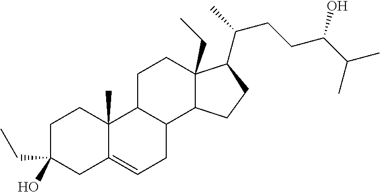 | B | N/A |
| F-6 | 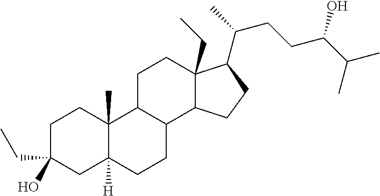 | B | N/A |
| F-7 | 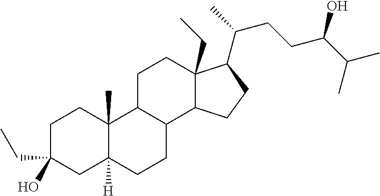 | B | N/A |
| F-8 | 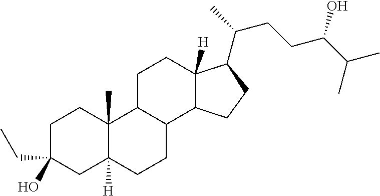 | B | N/A |
| F-9 | 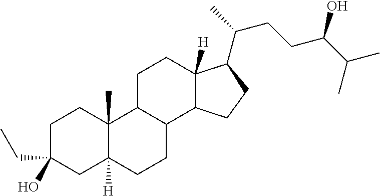 | B | N/A |
| F-10 | 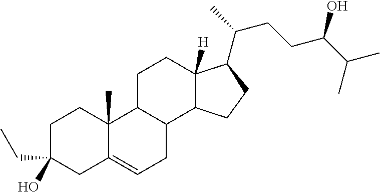 | B | N/A |
| F-14 | 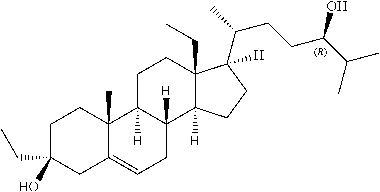 | B | N/A |

For Table F-2, “A” indicates an EC50 of 1 to 100 nM; “B” indicates an EC50 of greater than 100 nM up to 500 nM; “C” indicates an EC50 greater than 500 nM up to 1 μM; “D” indicates an EC50 of greater than 1 μM; “F” indicates an Emax of up to 100%; “G” indicates an Emax between 100% and 200%; and “H” indicates an Emax greater than 200%.

TABLE G-2

| Cmpd | Structure | EC50 (nM) | Emax |
|---|---|---|---|
| G-1 | 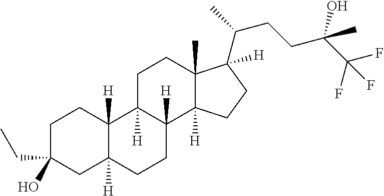 | A | H |
| G-2 | 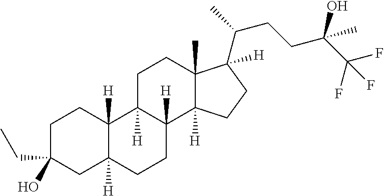 | C | G |
| G-3 | 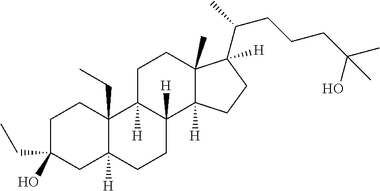 | A | G |
| G-4 | 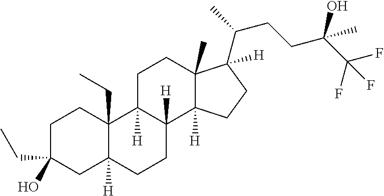 | B | G |
| G-5 | 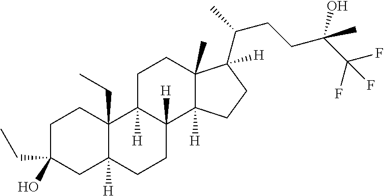 | A | H |

For Table G-2, “A” indicates an EC50 of 1 to 100 nM, “B” indicates an EC50 of greater than 100 nM up to 500 nM, “C” indicates an EC50 greater than 500 nM up to 1 μM; “D” indicates an EC50 of greater than 1 μM, “F” indicates an Emax of up to 100%, and “G” indicates an Emax between 100% and 200%, and “H” indicates an Emax greater than 200%.

TABLE H-2

| Compound | Structure | EC50 (nM) | Emax |
|---|---|---|---|
| H-1 | 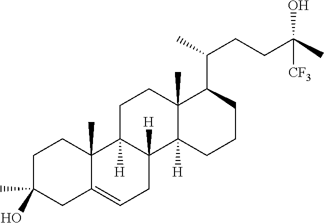 | C | G |
| H-2 | 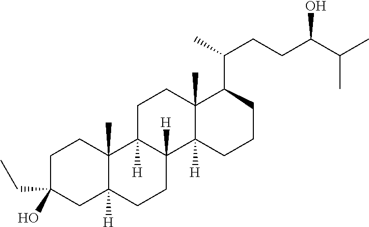 | | N/A |
| H-3 | 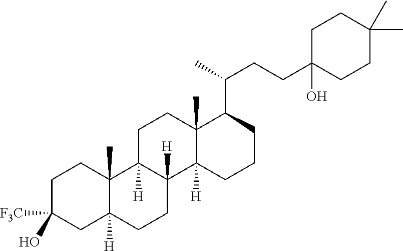 | D | N/A |
| H-4 | 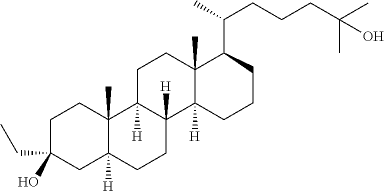 | B | G |
| H-5 | 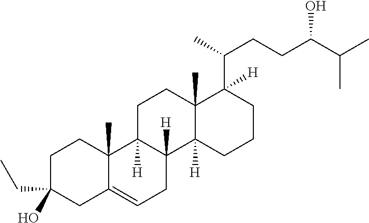 | B | N/A |
| H-6 | 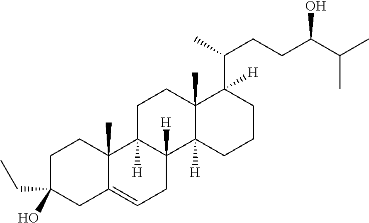 | A | F |
| H-7 | 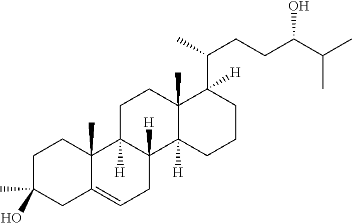 | B | N/A |
| H-8 | 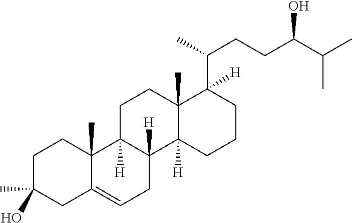 | B | F |
| H-9 | 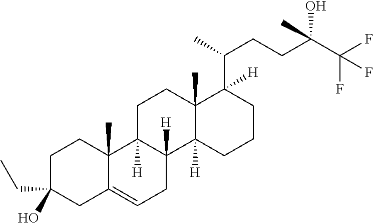 | B | N/A |
| H-10 | 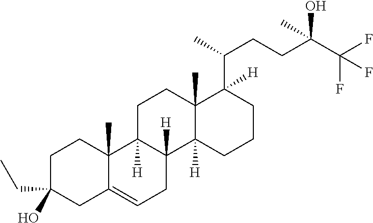 | C | N/A |
| H-11 | 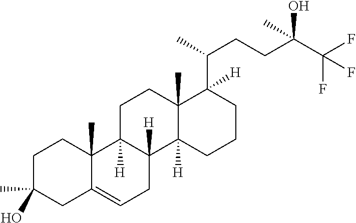 | B | F |

For Table H-2, “A” indicates an EC50 of 1 to 100 nM, “B” indicates an EC50 of greater than 100 nM up to 500 nM, “C” indicates an EC50 greater than 500 nM up to 1 μM; “D” indicates an EC50 of greater than 1 μM; “F” indicates an Emax of up to 100%, and “G” indicates an Emax between 100% and 200%, and “H” indicates an Emax greater than 200%.

### Numbered Embodiments

The disclosure also includes the following embodiments:

A-1. A compound of Formula (A-I):

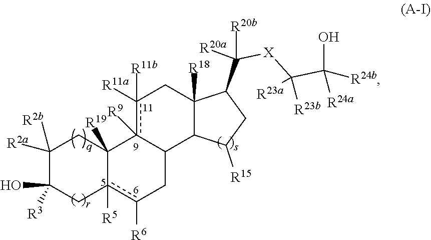

or a pharmaceutically acceptable salt thereof;
wherein:
   each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein R^{A2} is hydrogen or substituted or unsubstituted C₁₋₆alkyl;
   R³ is substituted or unsubstituted C₁₋₆alkyl, substituted or unsubstituted C₂₋₆ alkenyl, substituted or unsubstituted C₂₋₆alkynyl, or substituted or unsubstituted C₃₋₁₀ carbocyclyl;
   R⁵ is absent, hydrogen, halogen, or substituted or unsubstituted C₁₋₆alkyl;
   R⁶ is hydrogen, halogen, or substituted or unsubstituted C₁₋₆alkyl;
   R⁹ is absent or hydrogen; or
   R⁹ and Rla are taken together with the carbon atoms to which they are attached to form a substituted or unsubstituted C₃₋₆carbocyclyl ring;
   R^{11a} is hydrogen, halogen, substituted or unsubstituted C₁₋₆alkyl, or —OR^{A11}, wherein R^{A11} is hydrogen or substituted or unsubstituted C₁₋₆alkyl;
   R^{11b} is absent or hydrogen; or
   R^{11a} and R^{11b} are joined to form an oxo (═O) group;
   each instance of R¹⁵ is hydrogen, halogen, —OH, —NH₂, substituted or unsubstituted C₁₋₆alkyl, substituted or unsubstituted C₂₋₆alkenyl, substituted or unsubstituted C₂₋₆alkynyl, substituted or unsubstituted C₁₋₆alkoxy, substituted or unsubstituted C₃₋₆carbocyclyl, or substituted or unsubstituted 3- to 10-membered heterocyclyl;
   R¹⁶ is hydrogen, halogen, —OH, —NH₂, substituted or unsubstituted C₁₋₆alkyl, substituted or unsubstituted C₂₋₆alkenyl, substituted or unsubstituted C₂₋₆alkynyl, substituted or unsubstituted C₁₋₆alkoxy, substituted or unsubstituted C₃₋₆carbocyclyl, or substituted or unsubstituted 3- to 10-membered heterocyclyl; or
   R¹⁵ and R¹⁶ are taken together with the carbon atoms to which they are attached, to form a substituted or unsubstituted C₃₋₆carbocyclyl;
   R¹⁸ is hydrogen, substituted or unsubstituted C₁₋₆alkyl, or OR^{A18}, wherein each instance of R^{A18} is independently substituted or unsubstituted C₁₋₆alkyl;
   R¹⁹ is hydrogen, substituted or unsubstituted C₁₋₆alkyl, or OR^{A19}; wherein each instance of R^{A19} is independently substituted or unsubstituted C₁₋₆alkyl;
   R^{20a} is halogen, cyano, substituted C₁₋₆alkyl, unsubstituted C₂₋₆alkyl, substituted or unsubstituted C₁₋₆alkoxy, or substituted or unsubstituted C₃₋₆carbocyclyl;
   R^{20b} is hydrogen, halogen, cyano, substituted or unsubstituted C₁₋₆alkyl, substituted or unsubstituted C₁₋₆alkoxy, or substituted or unsubstituted C₃₋₆carbocyclyl;
   or R^{20a} and R^{20b} are both simultaneously —CH₃;
   X is —(C(R^{X})₂)ₙ— or —O—, wherein R^{X} is hydrogen or fluorine, or one R^{X} group and R^{23b} are joined to form a double bond;
   n is 1, 2 or 3;
   each instance of R^{23a} and R^{23b} is independently hydrogen or fluorine;
   R^{24a} is selected from the group consisting of substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl;
   R^{24b} is hydrogen, substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, or substituted or unsubstituted 5- to 10-membered heteroaryl;
   or R^{24a} and R^{24b}, taken together with the carbon atom to which they are attached, form a substituted or unsubstituted C₃₋₆carbocyclyl or a substituted or unsubstituted 3- to 10-membered heterocyclyl;
   q is 0, 1, or 2;
   r is 1 or 2;
   s is 1, 2, or 3; and
   each [symbol] independently represents a single or double bond, provided if the bond between C5 and C6 is a double bond, R⁵ is absent and if the bond between C9 and C11 is a double bond, R⁹ and R^{11b} are absent.

A-2. The compound or pharmaceutically acceptable salt thereof according to embodiment A-1, wherein the compound of formula (A-I) is a compound of formula (A-II):

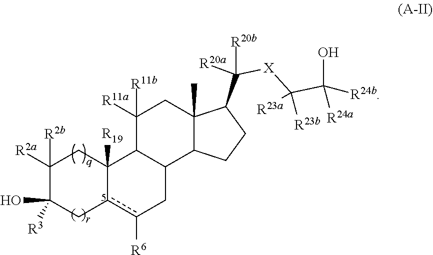

A-3. The compound or pharmaceutically acceptable salt thereof according to embodiment A-2, wherein the compound of formula (A-II) is a compound of formula (A-II-1) or formula (A-II-2):

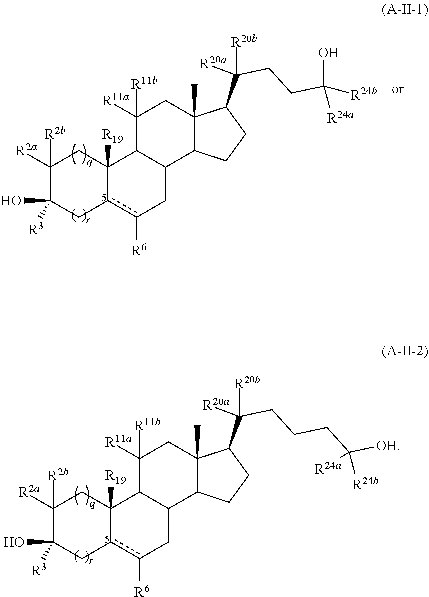

A-4. The compound or pharmaceutically acceptable salt thereof according to embodiment A-3, wherein the compound of formula (A-II-1) is a compound of formula (A-II-1-A) or formula (A-II-1-B):

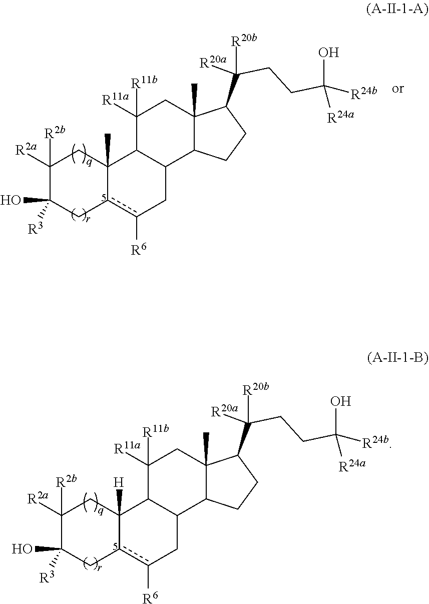

A-5. The compound or pharmaceutically acceptable salt thereof according to embodiment A-4, wherein the compound of formula (A-II-1-A) is a compound of formula (A-II-1-A-1) or formula (A-II-1-A-2):

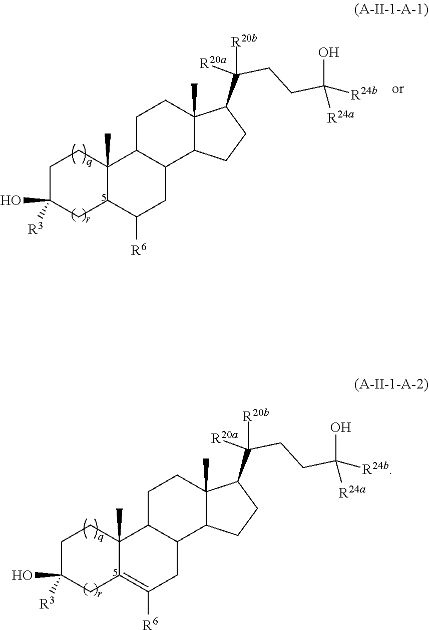

A-6. The compound or pharmaceutically acceptable salt thereof according to embodiment A-5, wherein the compound of formula (A-II-1-A-1) is a compound of formula (A-II-1-A-1-a):

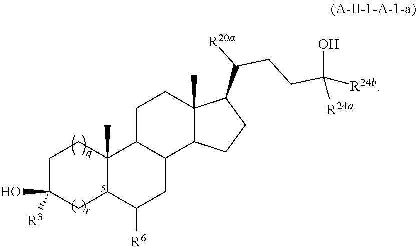

A-7. The compound or pharmaceutically acceptable salt thereof according to embodiment A-6, wherein the compound of formula (A-II-1-A-1-a) is a compound of formula (A-II-1-A-1-a-i) or (A-II-1-A-1-a-ii):

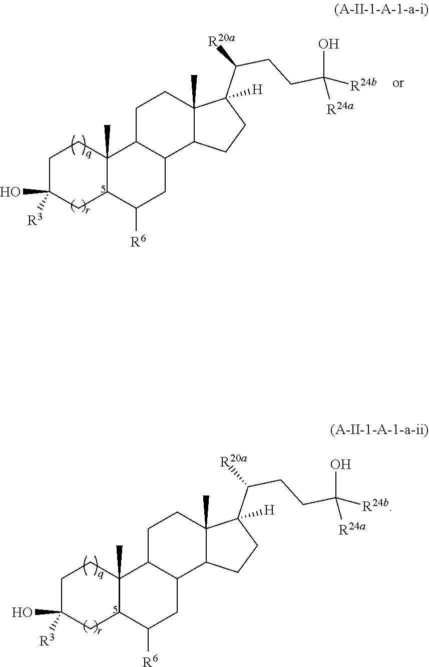

A-8. The compound or pharmaceutically acceptable salt thereof according to embodiment A-7, wherein the compound of formula (A-II-1-A-1-a-i) is a compound of formula (A-II-1-A-1-a-iii) or (A-II-1-A-1-a-iv):

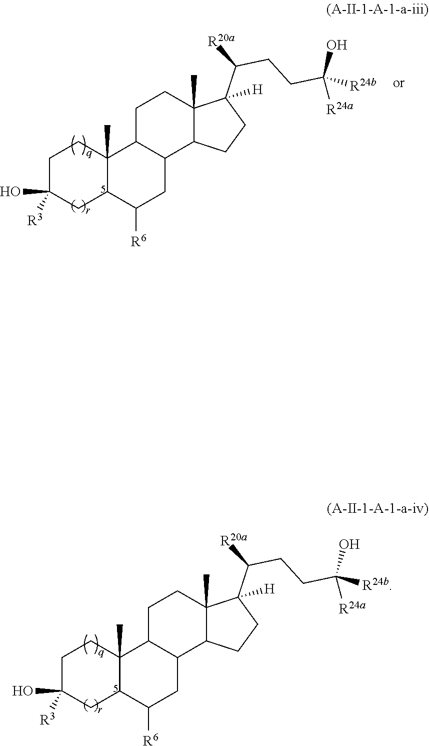

A-9. The compound or pharmaceutically acceptable salt thereof according to embodiment A-7, wherein the compound of formula (A-II-1-A-1-a-ii) is a compound of formula (A-II-1-A-1-a-v) or (A-II-1-A-1-a-vi):

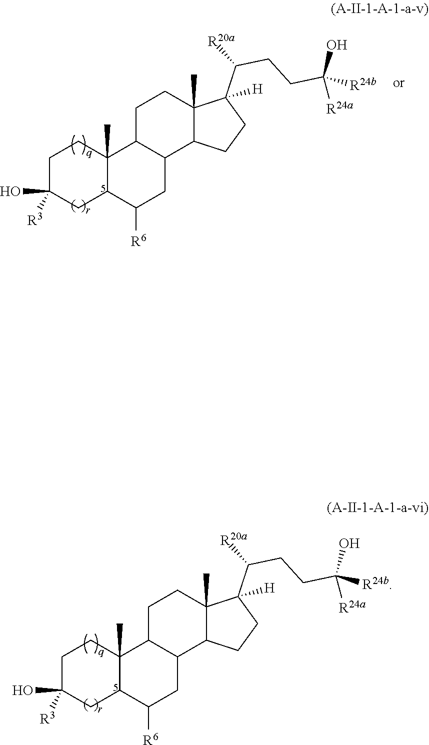

A-10. The compound or pharmaceutically acceptable salt thereof according to embodiment A-5, wherein the compound of formula (A-II-1-A-2) is a compound of formula (A-II-1-A-2-a):

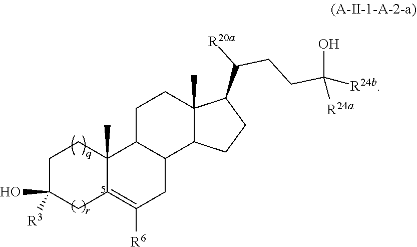

A-11. The compound or pharmaceutically acceptable salt thereof according to embodiment A-10, wherein the compound of formula (A-II-1-A-2-a) is a compound of formula (A-II-1-A-2-a-i) or (A-II-1-A-2-a-ii):

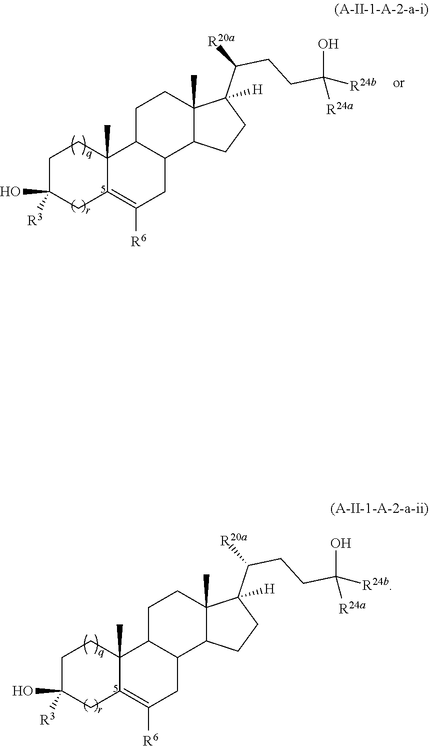

A-12. The compound or pharmaceutically acceptable salt thereof according to embodiment A-11, wherein the compound of formula (A-II-1-A-2-a-i) is a compound of formula (A-II-1-A-2-a-iii) or (A-II-1-A-2-a-iv):

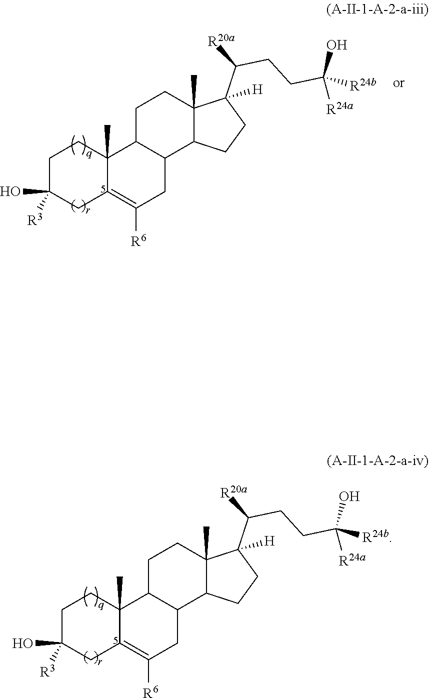

A-13. The compound or pharmaceutically acceptable salt thereof according to embodiment A-11, wherein the compound of formula (A-II-1-A-2-a-ii) is a compound of formula (A-II-1-A-2-a-v) or (A-II-1-A-2-a-vi):

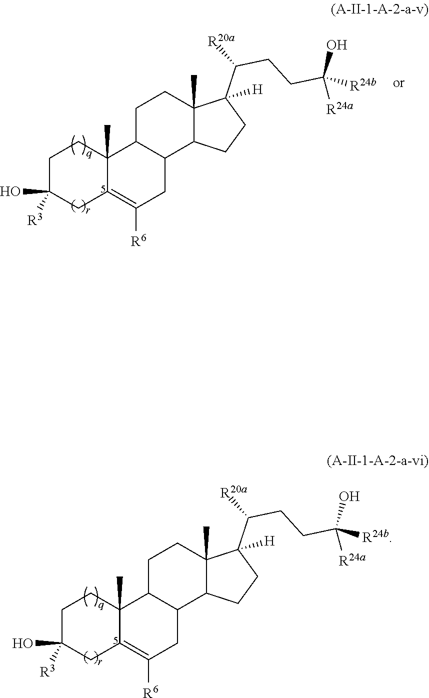

A-14. The compound or pharmaceutically acceptable salt thereof according to embodiment A-4, wherein the compound of formula (A-II-1-B) is a compound of formula (A-II-1-B-1) or formula (A-II-1-B-2):

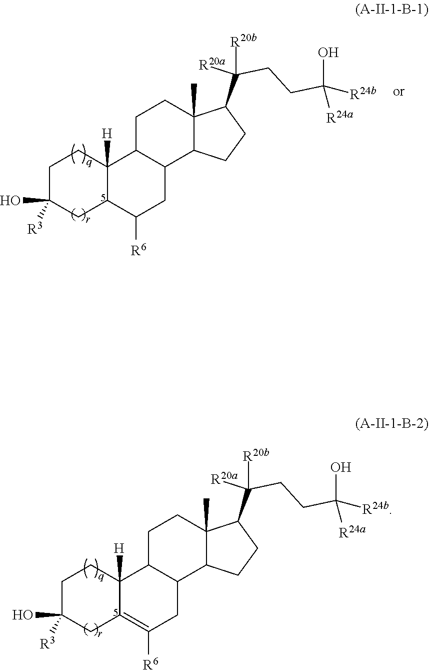

A-15. The compound or pharmaceutically acceptable salt thereof according to embodiment A-15, wherein the compound of formula (A-II-1-B-1) is a compound of formula (A-II-1-B-1-a):

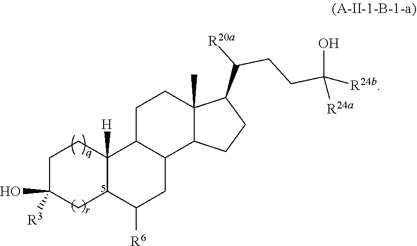

A-16. The compound or pharmaceutically acceptable salt thereof according to embodiment A-15, wherein the compound of formula (A-II-1-B-1-a) is a compound of formula (A-II-1-B-1-a-i) or formula (A-II-1-B-1-a-ii):

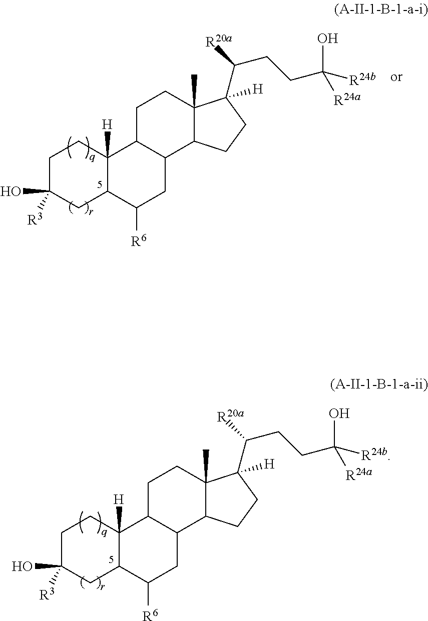

A-17. The compound or pharmaceutically acceptable salt thereof according to embodiment A-16, wherein the compound of formula (A-II-1-B-1-a-i) is a compound of formula (A-II-1-B-1-a-iii) or formula (A-II-1-B-1-a-iv):

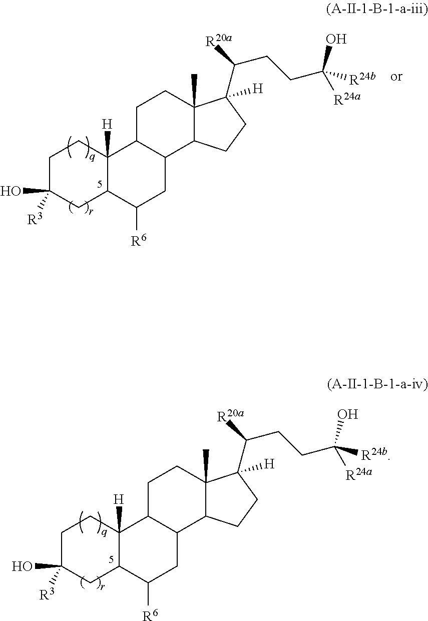

A-18. The compound or pharmaceutically acceptable salt thereof according to embodiment A-16, wherein the compound of formula (A-II-1-B-1-a-ii) is a compound of formula (A-II-1-B-1-a-v) or formula (A-II-1-B-1-a-vi):

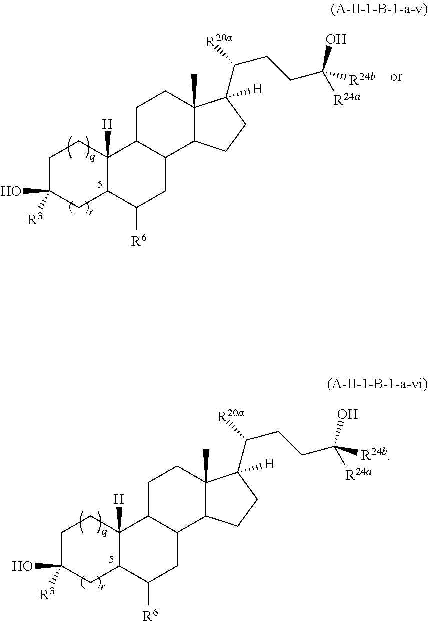

A-19. The compound or pharmaceutically acceptable salt thereof according to embodiment A-14, wherein the compound of formula (A-II-1-B-2) is a compound of formula (A-II-1-B-2-a):

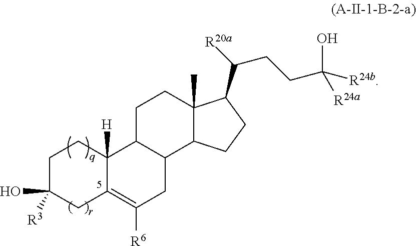

A-20. The compound or pharmaceutically acceptable salt thereof according to embodiment A-19, wherein the compound of formula (A-II-1-B-2-a) is a compound of formula (A-II-1-B-2-a-i) or formula (A-II-1-B-2-a-ii):

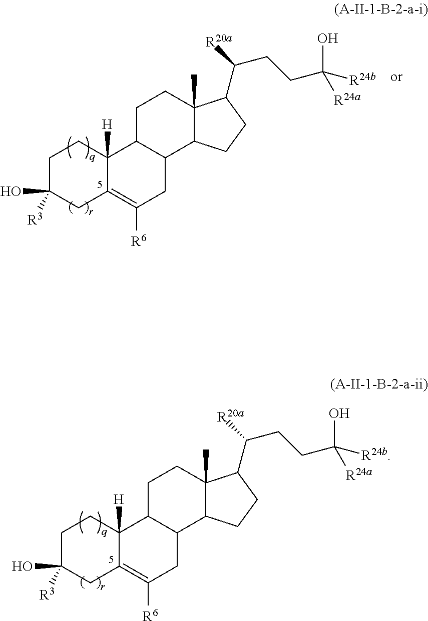

A-21. The compound or pharmaceutically acceptable salt thereof according to embodiment A-20, wherein the compound of formula (A-II-1-B-2-a-i) is a compound of formula (A-II-1-B-2-a-iii) or formula (A-II-1-B-2-a-iv):

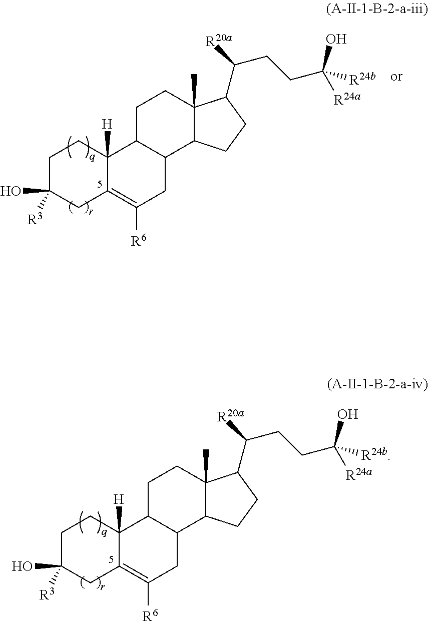

A-22. The compound or pharmaceutically acceptable salt thereof according to embodiment A-20, wherein the compound of formula (A-II-1-B-2-a-ii) is a compound of formula (A-II-1-B-2-a-v) or formula (A-II-1-B-2-a-vi):

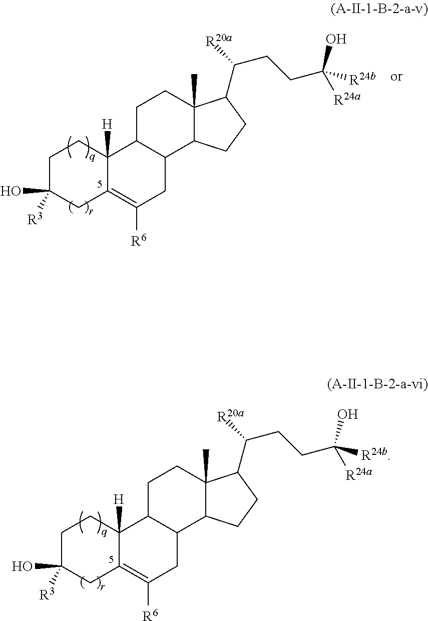

A-23. The compound or pharmaceutically acceptable salt thereof according to embodiment A-3, wherein the compound of formula (A-II-2) is a compound of formula (A-II-2-A) or formula (A-II-2-B):

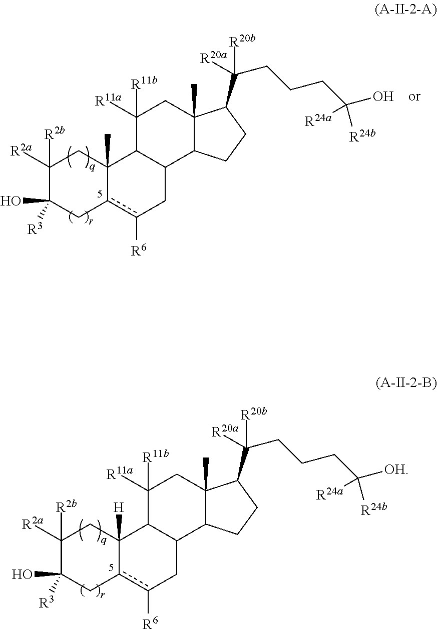

A-24. The compound or pharmaceutically acceptable salt thereof according to embodiment A-23, wherein the compound of formula (A-II-2-A) is a compound of formula (A-II-2-A-1) or formula (A-II-2-A-2):

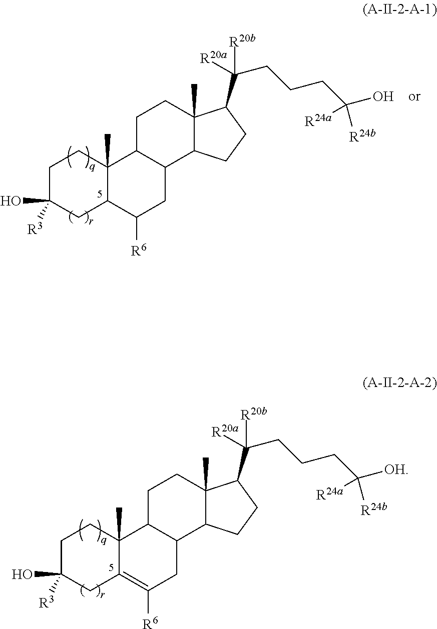

A-25. The compound or pharmaceutically acceptable salt thereof according to embodiment A-24, wherein the compound of formula (A-II-2-A-1) is a compound of formula (A-II-2-A-1-a):

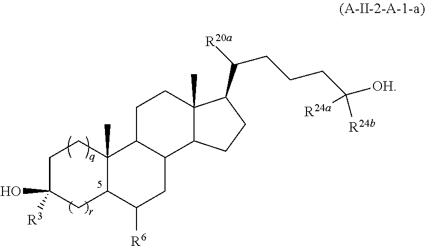

A-26. The compound or pharmaceutically acceptable salt thereof according to embodiment A-25, wherein the compound of formula (A-II-2-A-1-a) is a compound of formula (A-II-2-A-1-a-i) or formula (A-II-2-A-1-a-ii):

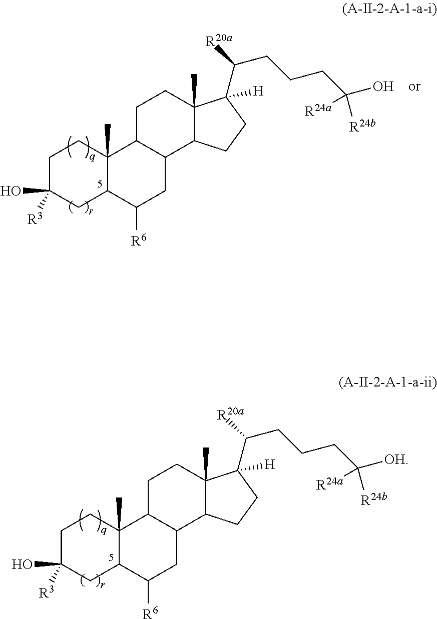

A-27. The compound or pharmaceutically acceptable salt thereof according to embodiment A-26, wherein the compound of formula (A-II-2-A-1-a-i) is a compound of formula (A-II-2-A-1-a-iii) or formula (A-II-2-A-1-a-iv):

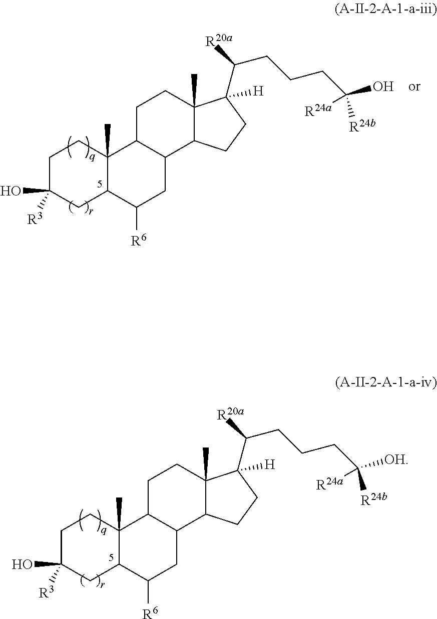

A-28. The compound or pharmaceutically acceptable salt thereof according to embodiment A-26, wherein the compound of formula (A-II-2-A-1-a-ii) is a compound of formula (A-II-2-A-1-a-v) or formula (A-II-2-A-1-a-vi):

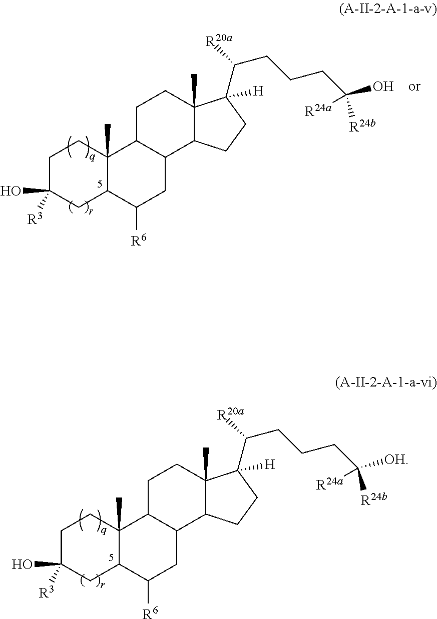

A-29. The compound or pharmaceutically acceptable salt thereof according to embodiment A-24, wherein the compound of formula (A-II-2-A-2) is a compound of formula (A-II-2-A-2-a):

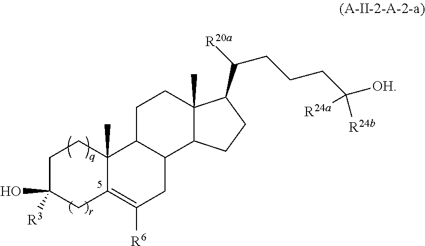

A-30. The compound or pharmaceutically acceptable salt thereof according to embodiment A-29, wherein the compound of formula (A-II-2-A-2-a) is a compound of formula (A-II-2-A-2-a-i) or formula (A-II-2-A-2-a-ii):

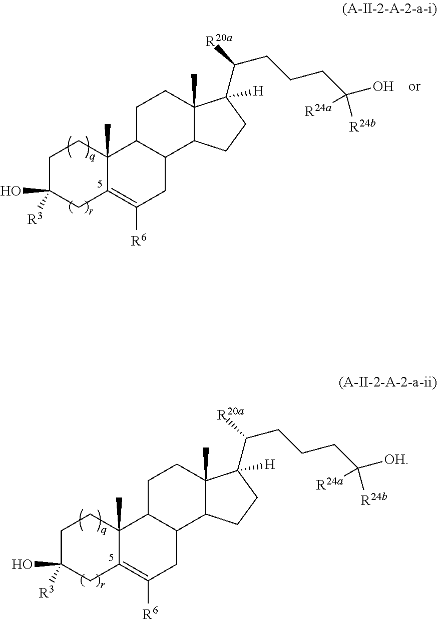

A-31. The compound or pharmaceutically acceptable salt thereof according to embodiment A-30, wherein the compound of formula (A-II-2-A-2-a-i) is a compound of formula (A-II-2-A-2-a-iii) or formula (A-II-2-A-2-a-iv):

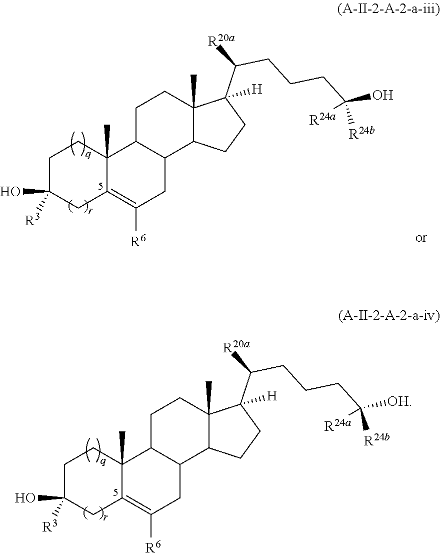

A-32. The compound or pharmaceutically acceptable salt thereof according to embodiment A-30, wherein the compound of formula (A-II-2-A-2-a-ii) is a compound of formula (A-II-2-A-2-a-v) or formula (A-II-2-A-2-a-vi):

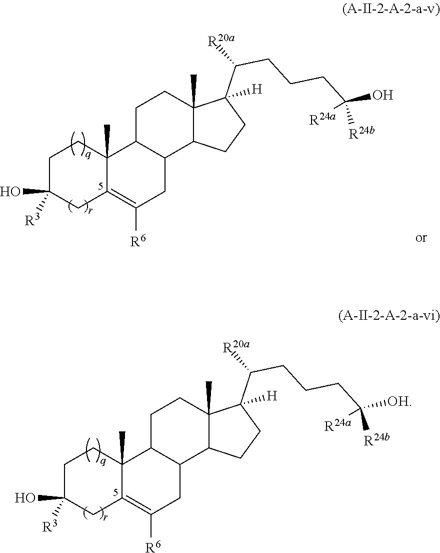

A-33. The compound or pharmaceutically acceptable salt thereof according to embodiment A-23, wherein the compound of formula (A-II-2-B) is a compound of formula (A-II-2-B-1) or formula (A-II-2-B-2):

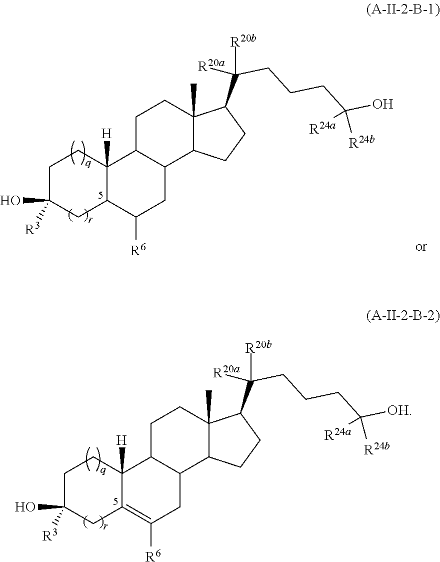

A-34. The compound or pharmaceutically acceptable salt thereof according to embodiment A-33, wherein the compound of formula (A-II-2-B-1) is a compound of formula (A-II-2-B-1-a):

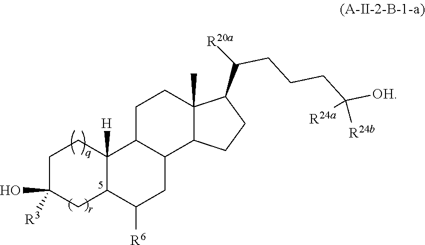

A-35. The compound or pharmaceutically acceptable salt thereof according to embodiment A-34, wherein the compound of formula (A-II-2-B-1-a) is a compound of formula (A-II-2-B-1-a-i) or formula (A-II-2-B-1-a-ii):

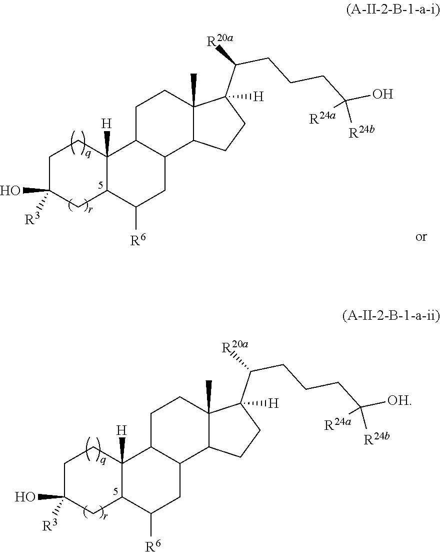

A-36. The compound or pharmaceutically acceptable salt thereof according to embodiment A-35, wherein the compound of formula (A-II-2-B-1-a-i) is a compound of formula (A-II-2-B-1-a-iii) or formula (A-II-2-B-1-a-iv):

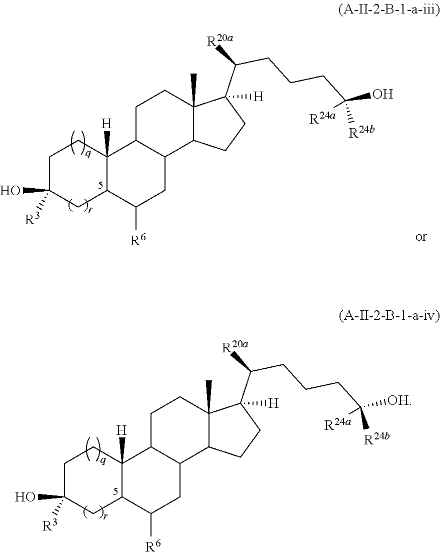

A-37. The compound or pharmaceutically acceptable salt thereof according to embodiment A-35, wherein the compound of formula (A-II-2-B-1-a-ii) is a compound of formula (A-II-2-B-1-a-v) or formula (A-II-2-B-1-a-vi):

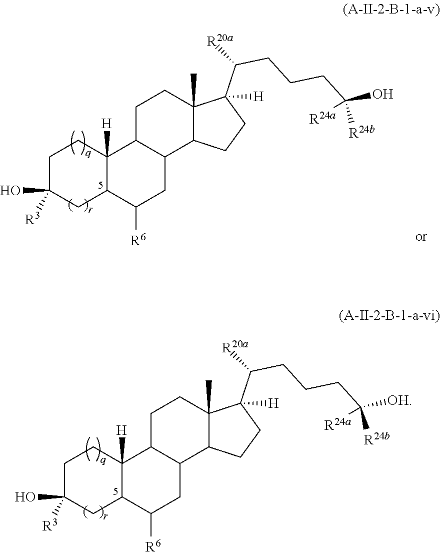

A-38. The compound or pharmaceutically acceptable salt thereof according to embodiment A-33, wherein the compound of formula (A-II-2-B-2) is a compound of formula (A-II-2-B-2-a):

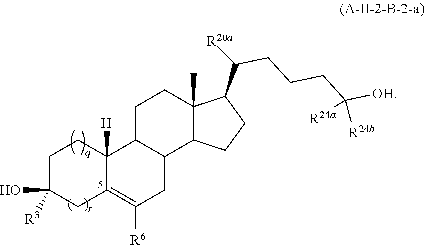

A-39. The compound or pharmaceutically acceptable salt thereof according to embodiment A-38, wherein the compound of formula (A-II-2-B-2-a) is a compound of formula (A-II-2-B-2-a-i) or formula (A-II-2-B-2-a-ii):

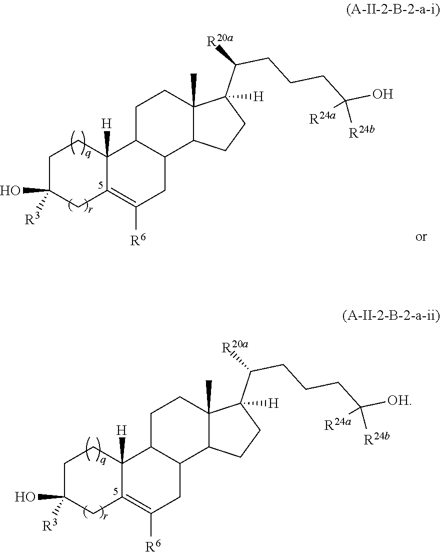

A-40. The compound or pharmaceutically acceptable salt thereof according to embodiment A-39, wherein the compound of formula (A-II-2-B-2-a-i) is a compound of formula (A-II-2-B-2-a-iii) or formula (A-II-2-B-2-a-iv):

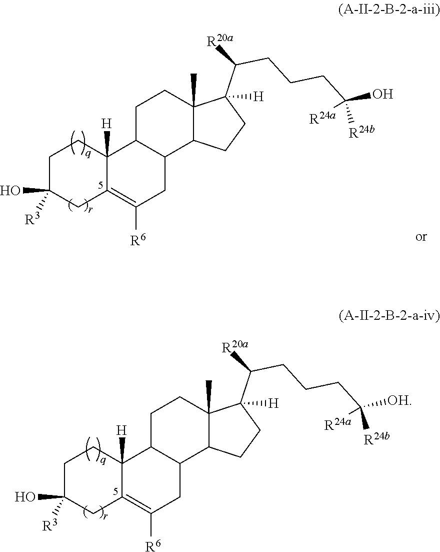

A-41. The compound or pharmaceutically acceptable salt thereof according to embodiment A-39, wherein the compound of formula (A-II-2-B-2-a-ii) is a compound of formula (A-II-2-B-2-a-v) or formula (A-II-2-B-2-a-vi):

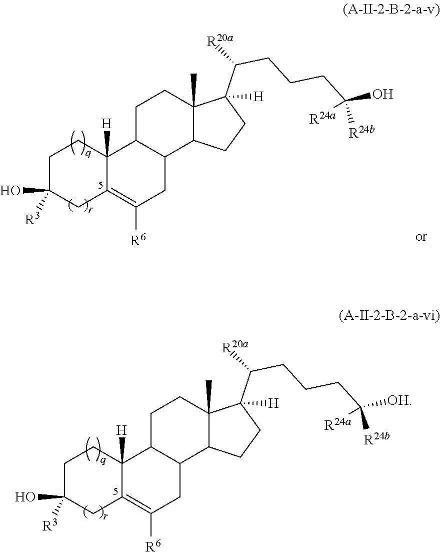

A-42. The compound or pharmaceutically acceptable salt thereof according to embodiment A-1, wherein the compound of formula (A-I) is a compound of formula (A-III):

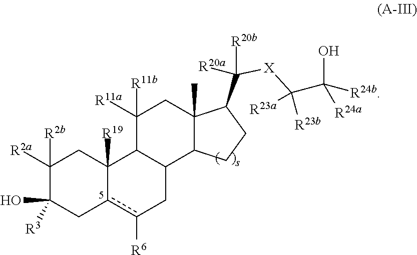

A-43. The compound or pharmaceutically acceptable salt thereof according to embodiment A-42, wherein the compound of formula (A-III) is a compound of formula (A-III-1) or formula (A-III-2):

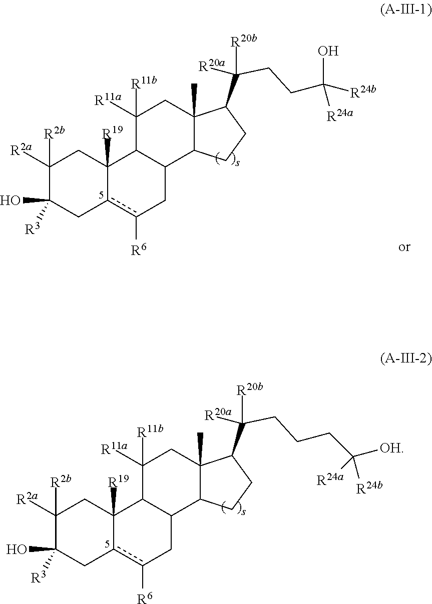

A-44. The compound or pharmaceutically acceptable salt thereof according to embodiment A-43, wherein the compound of formula (A-III-1) is a compound of formula (A-III-1-A) or formula (A-III-1-B):

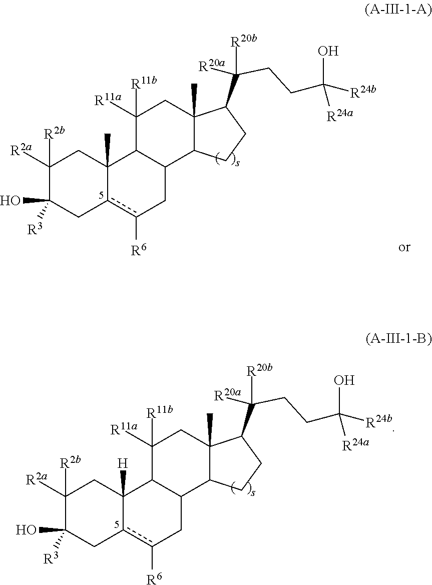

A-45. The compound or pharmaceutically acceptable salt thereof according to embodiment A-44, wherein the compound of formula (A-III-1-A) is a compound of formula (A-III-1-A-1) or formula (A-III-1-A-2):

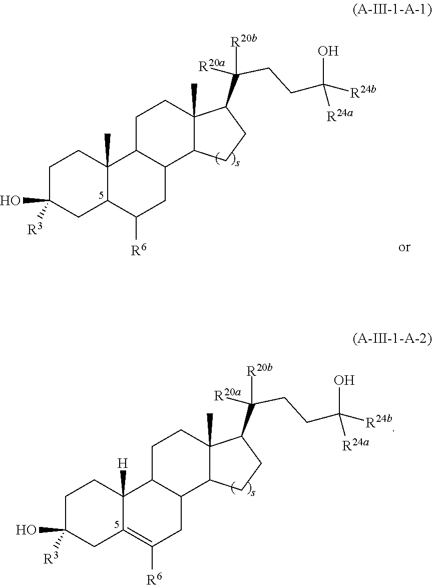

A-46. The compound or pharmaceutically acceptable salt thereof according to embodiment A-45, wherein the compound of formula (A-III-1-A-1) is a compound of formula (A-III-1-A-1-a):

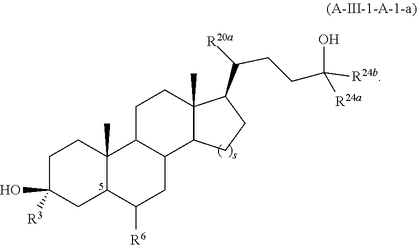

A-47. The compound or pharmaceutically acceptable salt thereof according to embodiment A-46, wherein the compound of formula (A-III-1-A-1-a) is a compound of formula (A-III-1-A-1-a-i) or (A-III-1-A-1-a-ii):

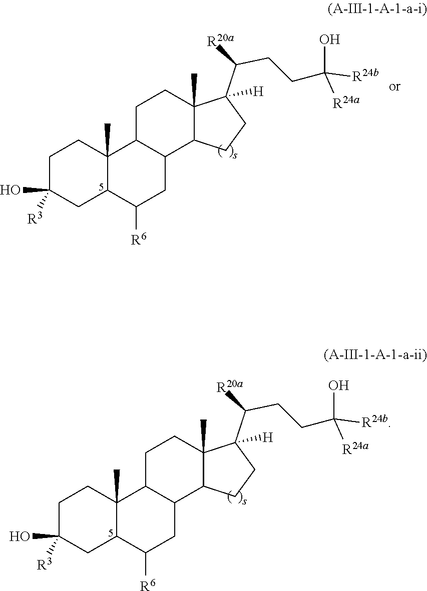

A-48. The compound or pharmaceutically acceptable salt thereof according to embodiment A-47, wherein the compound of formula (A-III-1-A-1-a-i) is a compound of formula (A-III-1-A-1-a-iii) or (A-III-1-A-1-a-iv):

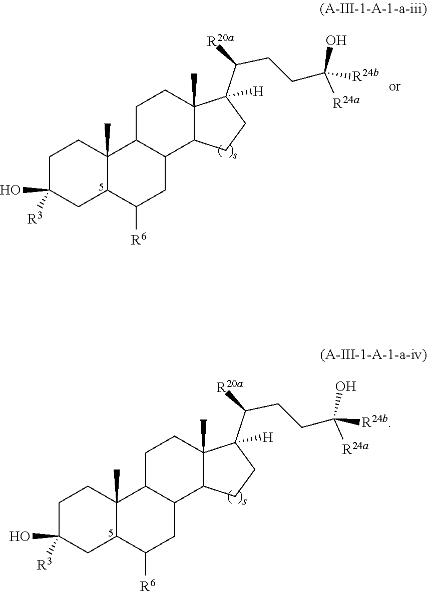

A-49. The compound or pharmaceutically acceptable salt thereof according to embodiment A-47, wherein the compound of formula (A-III-1-A-1-a-ii) is a compound of formula (A-III-1-A-1-a-v) or (A-III-1-A-1-a-vi):

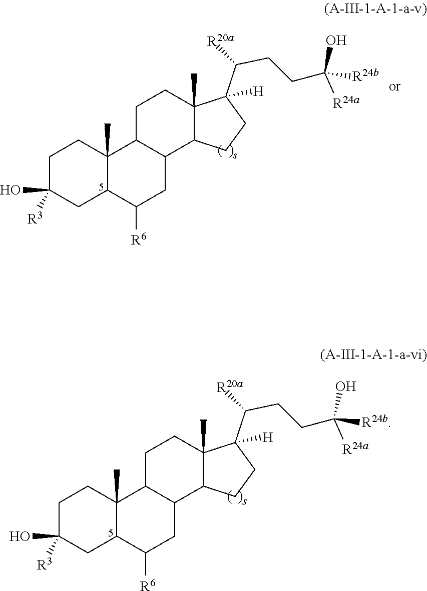

A-50. The compound or pharmaceutically acceptable salt thereof according to embodiment A-45, wherein the compound of formula (A-III-1-A-2) is a compound of formula (A-III-1-A-2-a):

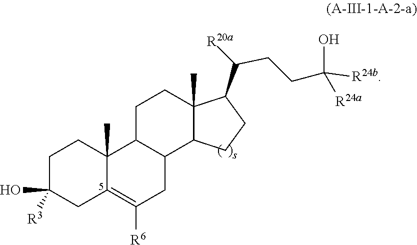

A-51. The compound or pharmaceutically acceptable salt thereof according to embodiment A-50, wherein the compound of formula (A-III-1-A-2-a) is a compound of formula (A-III-1-A-2-a-i) or (A-III-1-A-2-a-ii):

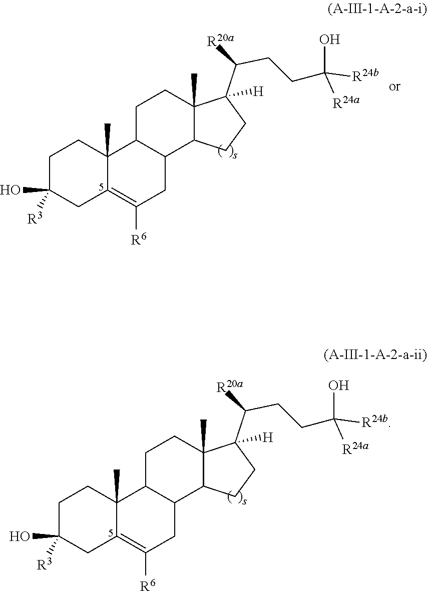

A-52. The compound or pharmaceutically acceptable salt thereof according to embodiment A-51, wherein the compound of formula (A-III-1-A-2-a-i) is a compound of formula (A-III-1-A-2-a-iii) or (A-III-1-A-2-a-iv):

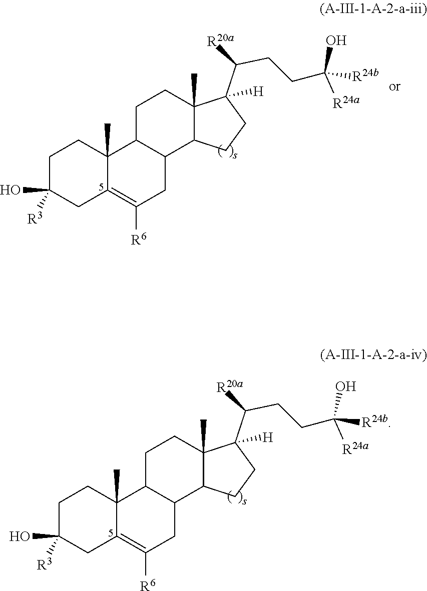

A-53. The compound or pharmaceutically acceptable salt thereof according to embodiment A-51, wherein the compound of formula (A-III-1-A-2-a-ii) is a compound of formula (A-III-1-A-2-a-v) or (A-III-1-A-2-a-vi):

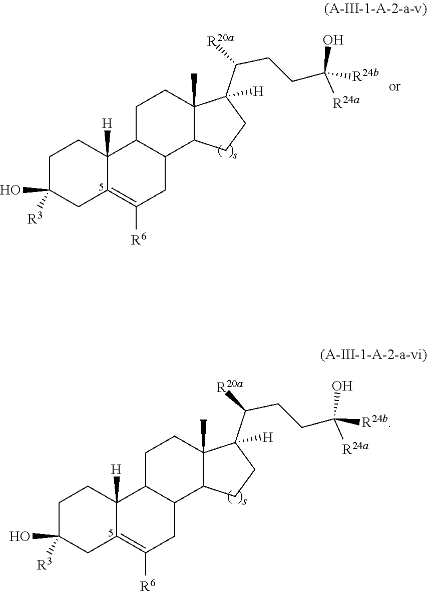

A-54. The compound or pharmaceutically acceptable salt thereof according to embodiment A-44, wherein the compound of formula (A-III-1-B) is a compound of formula (A-III-1-B-1) or formula (A-III-1-B-2):

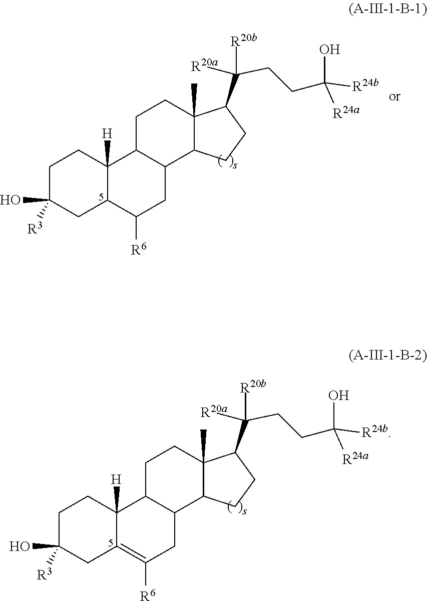

A-55. The compound or pharmaceutically acceptable salt thereof according to embodiment A-54, wherein the compound of formula (A-III-1-B-1) is a compound of formula (A-III-1-B-1-a):

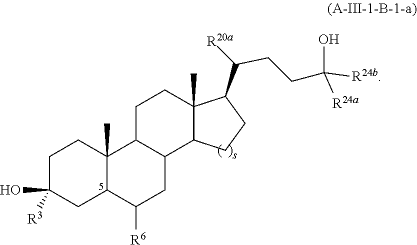

A-56. The compound or pharmaceutically acceptable salt thereof according to embodiment A-55, wherein the compound of formula (A-III-1-B-1-a) is a compound of formula (A-III-1-B-1-a-i) or formula (A-III-1-B-1-a-ii):

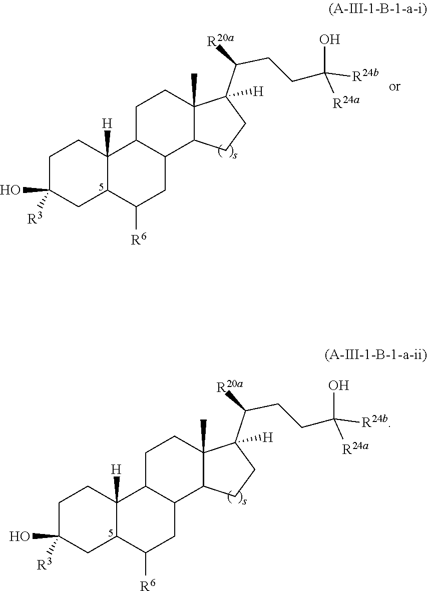

A-57. The compound or pharmaceutically acceptable salt thereof according to embodiment A-56, wherein the compound of formula (A-III-1-B-1-a-i) is a compound of formula (A-III-1-B-1-a-iii) or formula (A-III-1-B-1-a-iv):

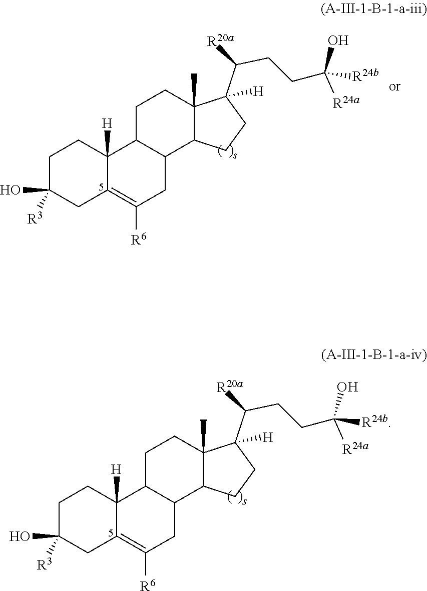

A-58. The compound or pharmaceutically acceptable salt thereof according to embodiment A-56, wherein the compound of formula (A-III-1-B-1-a-ii) is a compound of formula (A-III-1-B-1-a-v) or formula (A-III-1-B-1-a-vi):

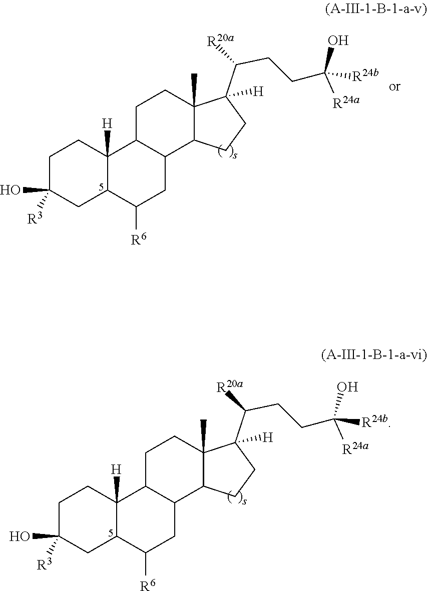

A-59. The compound or pharmaceutically acceptable salt thereof according to embodiment A-54, wherein the compound of formula (A-III-1-B-2) is a compound of formula (A-III-1-B-2-a):

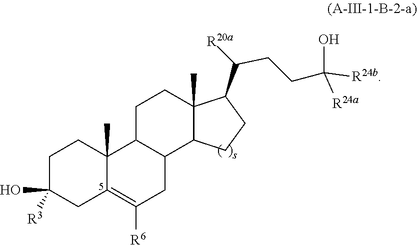

A-60. The compound or pharmaceutically acceptable salt thereof according to embodiment A-59, wherein the compound of formula (A-III-1-B-2-a) is a compound of formula (A-III-1-B-2-a-i) or formula (A-III-1-B-2-a-ii):

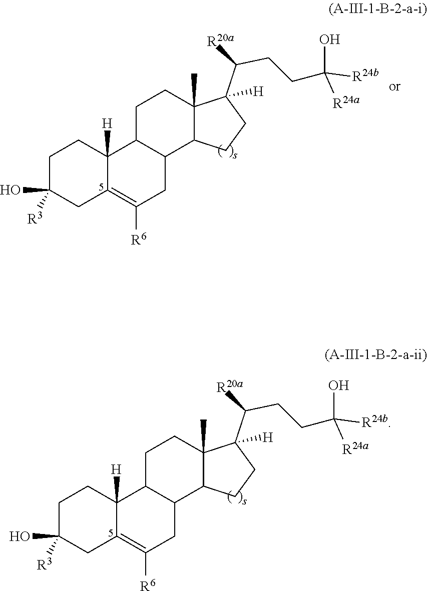

A-61. The compound or pharmaceutically acceptable salt thereof according to embodiment A-60, wherein the compound of formula (A-III-1-B-2-a-i) is a compound of formula (A-III-1-B-2-a-iii) or formula (A-III-1-B-2-a-iv):

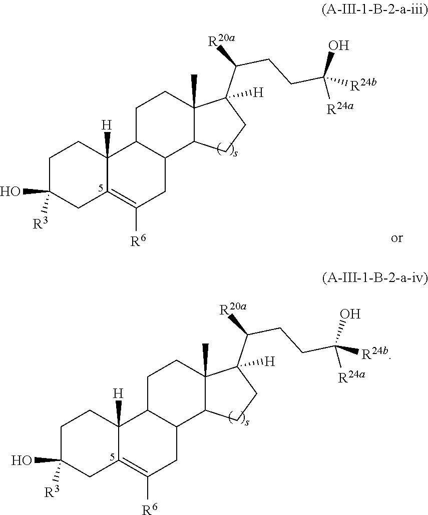

A-62. The compound or pharmaceutically acceptable salt thereof according to embodiment A-60, wherein the compound of formula (A-III-1-B-2-a-ii) is a compound of formula (A-III-1-B-2-a-v) or formula (A-III-1-B-2-a-vi):

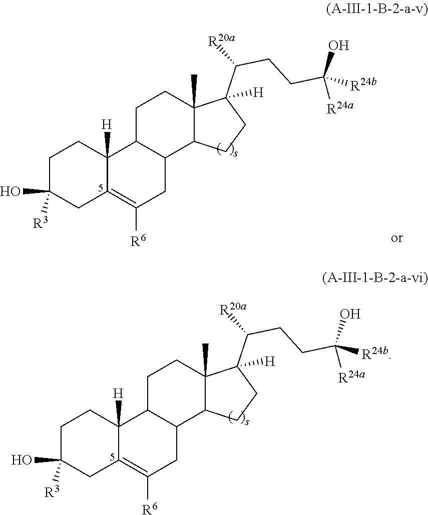

A-63. The compound or pharmaceutically acceptable salt thereof according to embodiment A-43, wherein the compound of formula (A-III-2) is a compound of formula (A-III-2-A) or formula (A-III-2-B):

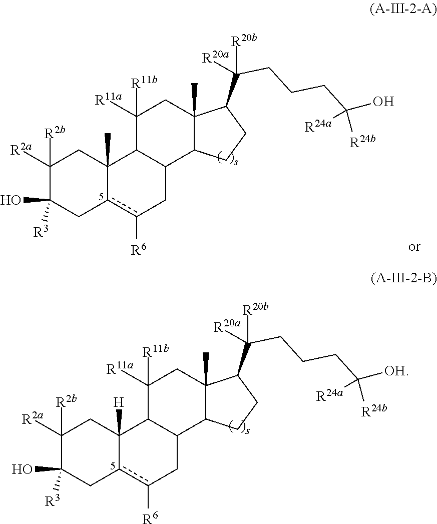

A-64. The compound or pharmaceutically acceptable salt thereof according to embodiment A-63, wherein the compound of formula (A-III-2-A) is a compound of formula (A-III-2-A-1) or formula (A-III-2-A-2):

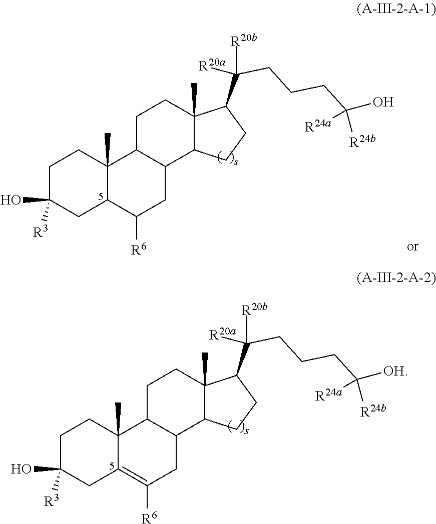

A-65. The compound or pharmaceutically acceptable salt thereof according to embodiment A-64, wherein the compound of formula (A-III-2-A-1) is a compound of formula (A-III-2-A-1-a):

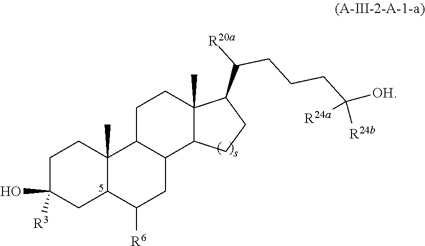

A-66. The compound or pharmaceutically acceptable salt thereof according to embodiment A-65, wherein the compound of formula (A-III-2-A-1-a) is a compound of formula (A-III-2-A-1-a-i) or formula (A-III-2-A-1-a-ii):

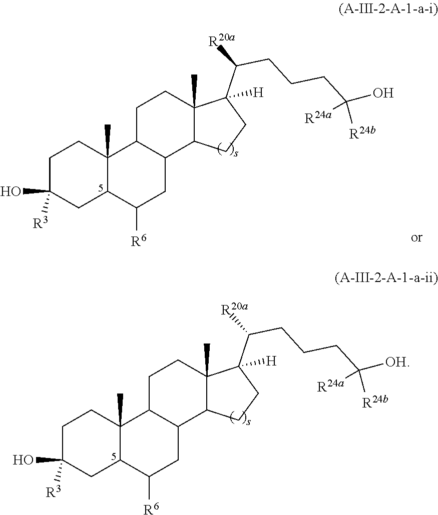

A-67. The compound or pharmaceutically acceptable salt thereof according to embodiment A-66, wherein the compound of formula (A-III-2-A-1-a-i) is a compound of formula (A-III-2-A-1-a-iii) or formula (A-III-2-A-1-a-iv):

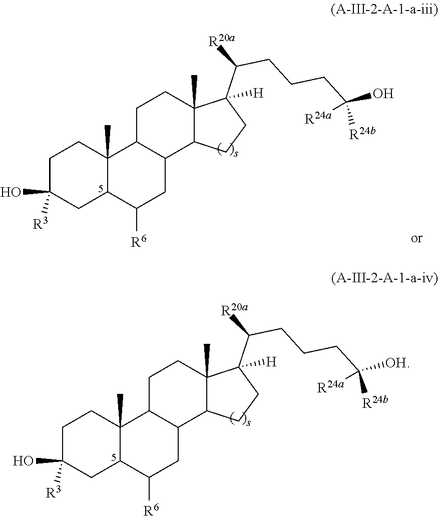

A-68. The compound or pharmaceutically acceptable salt thereof according to embodiment A-66, wherein the compound of formula (A-III-2-A-1-a-ii) is a compound of formula (A-III-2-A-1-a-v) or formula (A-III-2-A-1-a-vi):

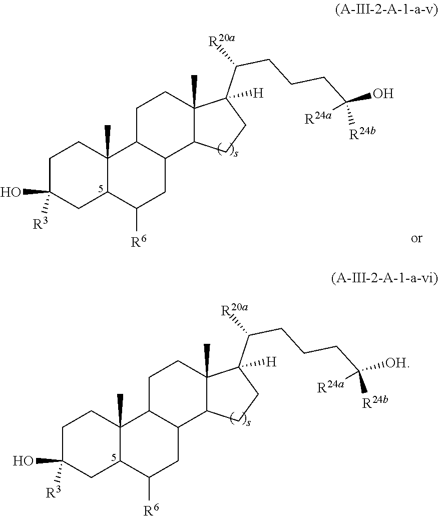

A-69. The compound or pharmaceutically acceptable salt thereof according to embodiment A-64, wherein the compound of formula (A-III-2-A-2) is a compound of formula (A-III-2-A-2-a):

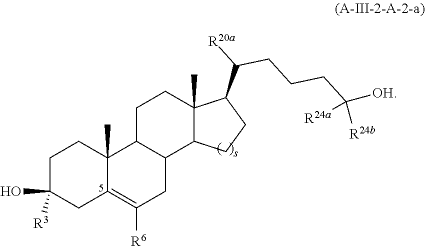

A-70. The compound or pharmaceutically acceptable salt thereof according to embodiment A-69, wherein the compound of formula (A-III-2-A-2-a) is a compound of formula (A-III-2-A-2-a-i) or formula (A-III-2-A-2-a-ii):

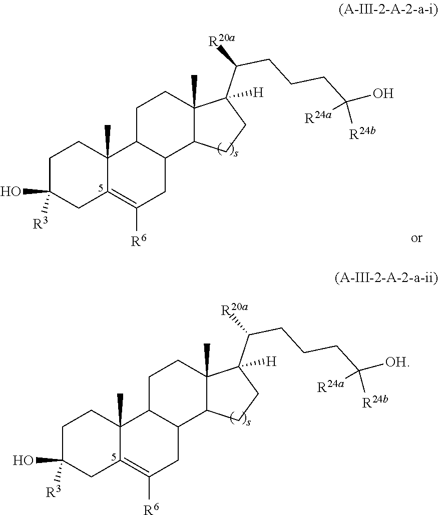

A-71. The compound or pharmaceutically acceptable salt thereof according to embodiment A-70, wherein the compound of formula (A-III-2-A-2-a-i) is a compound of formula (A-III-2-A-2-a-iii) or formula A-(III-2-A-2-a-iv):

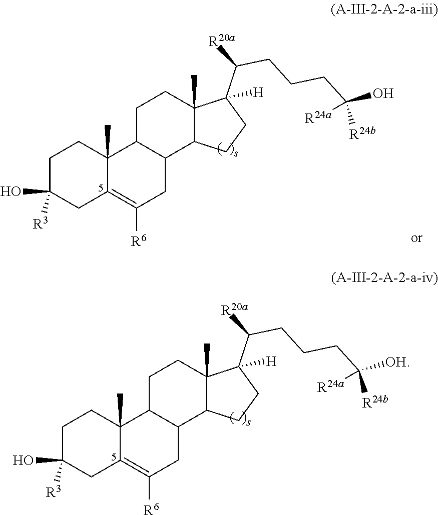

A-72. The compound or pharmaceutically acceptable salt thereof according to embodiment A-70, wherein the compound of formula (A-III-2-A-2-a-ii) is a compound of formula (A-III-2-A-2-a-v) or formula (A-III-2-A-2-a-vi):

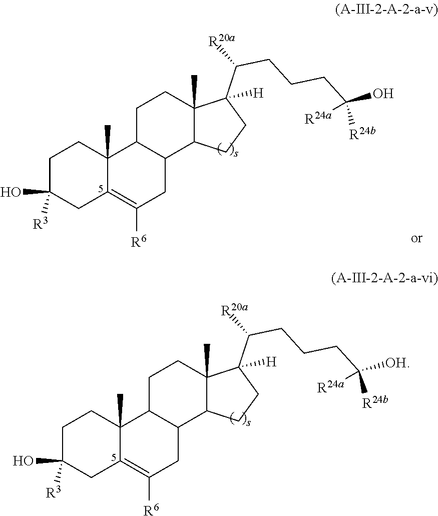

A-73. The compound or pharmaceutically acceptable salt thereof according to embodiment A-63, wherein the compound of formula (A-III-2-B) is a compound of formula (A-III-2-B-1) or formula (A-III-2-B-2):

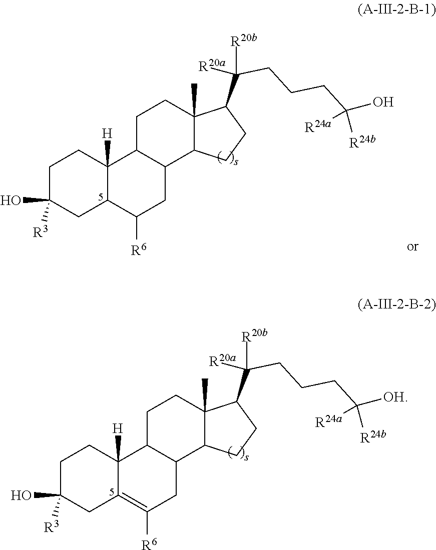

A-74. The compound or pharmaceutically acceptable salt thereof according to embodiment A-73, wherein the compound of formula (A-III-2-B-1) is a compound of formula (A-III-2-B-1-a):

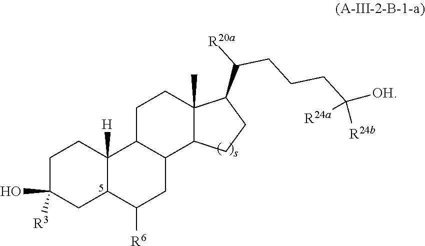

A-75. The compound or pharmaceutically acceptable salt thereof according to embodiment A-74, wherein the compound of formula (A-III-2-B-1-a) is a compound of formula (A-III-2-B-1-a-i) or formula (A-III-2-B-1-a-ii):

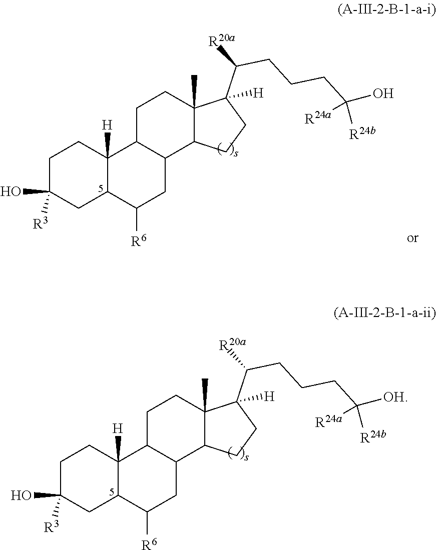

A-76. The compound or pharmaceutically acceptable salt thereof according to embodiment A-75, wherein the compound of formula (A-III-2-B-1-a-i) is a compound of formula (A-III-2-B-1-a-iii) or formula (A-III-2-B-1-a-iv):

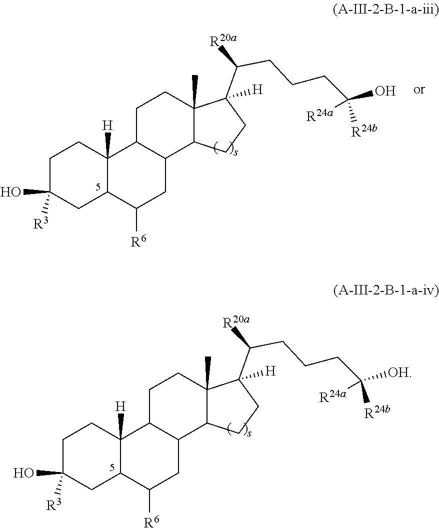

A-77. The compound or pharmaceutically acceptable salt thereof according to embodiment A-75, wherein the compound of formula (A-III-2-B-1-a-ii) is a compound of formula (A-III-2-B-1-a-v) or formula (A-III-2-B-1-a-vi):

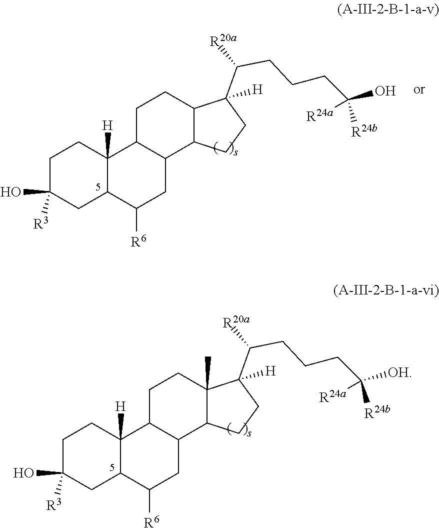

A-78. The compound or pharmaceutically acceptable salt thereof according to embodiment A-73, wherein the compound of formula (A-III-2-B-2) is a compound of formula (A-III-2-B-2-a):

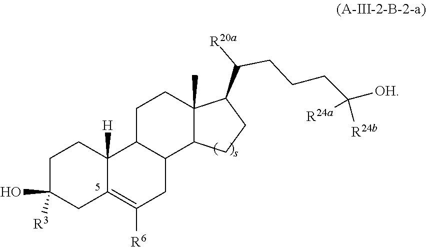

A-79. The compound or pharmaceutically acceptable salt thereof according to embodiment A-78, wherein the compound of formula (A-III-2-B-2-a) is a compound of formula (A-III-2-B-2-a-i) or formula (A-III-2-B-2-a-ii):

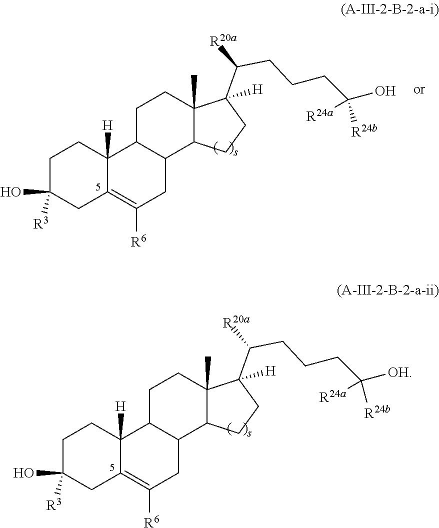

A-80. The compound or pharmaceutically acceptable salt thereof according to embodiment A-79, wherein the compound of formula (A-III-2-B-2-a-i) is a compound of formula (A-III-2-B-2-a-iii) or formula (A-III-2-B-2-a-iv):

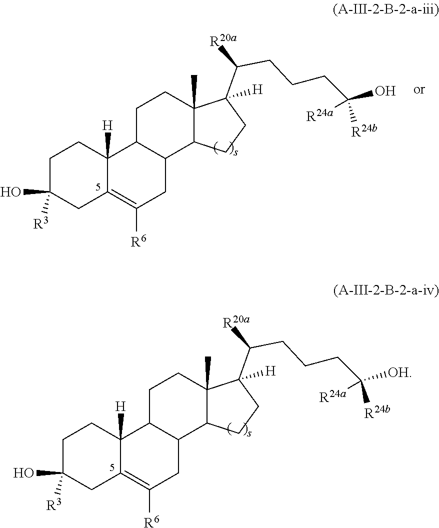

A-81. The compound or pharmaceutically acceptable salt thereof according to embodiment A-79, wherein the compound of formula (A-III-2-B-2-a-ii) is a compound of formula (A-III-2-B-2-a-v) or formula (A-III-2-B-2-a-vi):

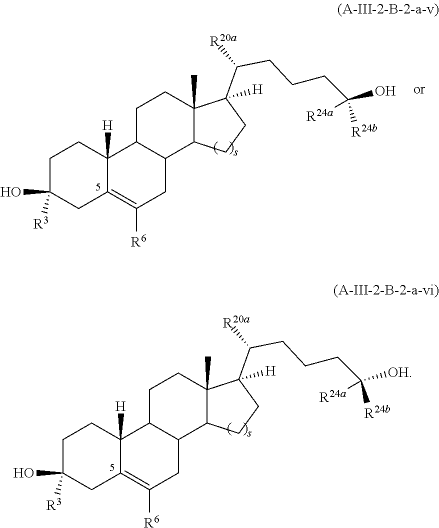

A-82. The compound or pharmaceutically acceptable salt thereof according to embodiment A-1, wherein the compound of formula (A-I) is a compound of formula (A-IV) or (A-V):

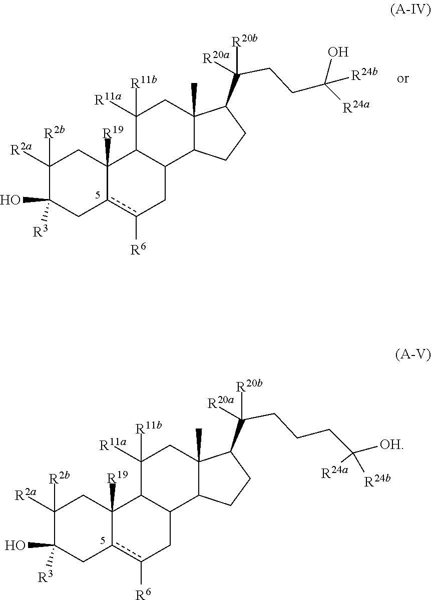

A-83. The compound or pharmaceutically acceptable salt thereof according to embodiment A-82, wherein the compound of formula (A-IV) is a compound of formula (A-IV-A) or (A-IV-B):

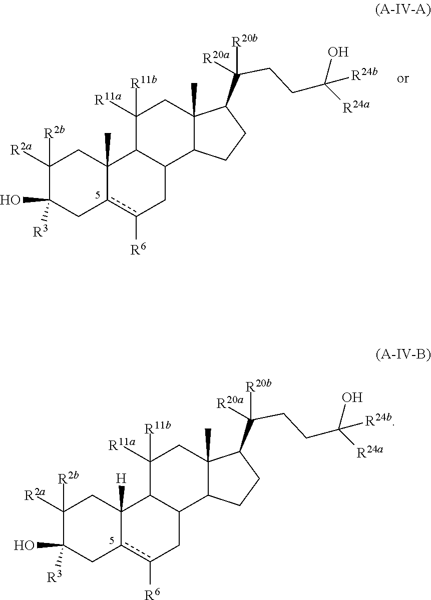

A-84. The compound or pharmaceutically acceptable salt thereof according to embodiment A-83, wherein the compound of formula (A-IV-A) is a compound of formula (A-IV-A-1) or (A-IV-A-2):

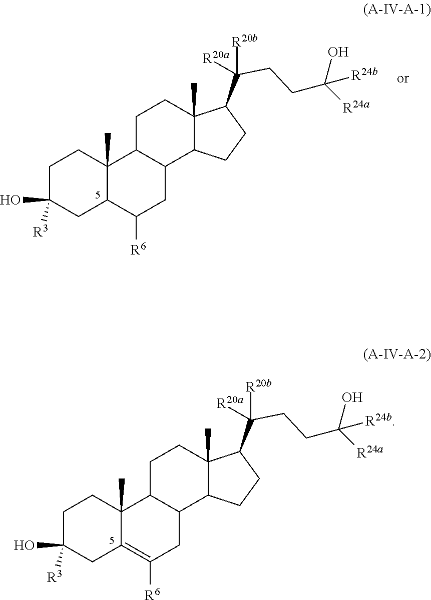

A-85. The compound or pharmaceutically acceptable salt thereof according to embodiment A-84, wherein the compound of formula (A-IV-A-1) is a compound of formula (A-IV-A-1-a):

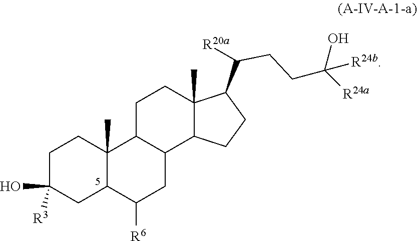

A-86. The compound or pharmaceutically acceptable salt thereof according to embodiment A-85, wherein the compound of formula (A-IV-A-1-a) is a compound of formula (A-IV-A-1-a-i) or (A-IV-A-1-a-ii):

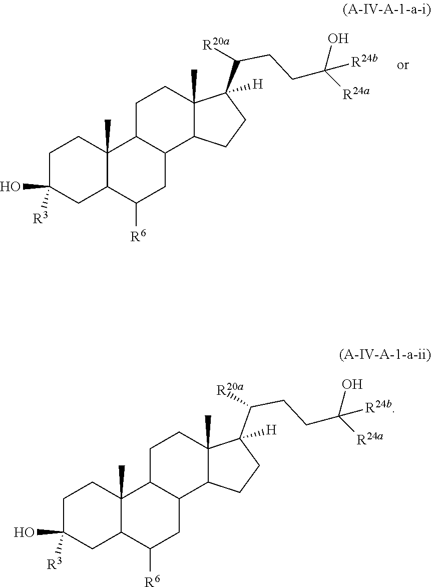

A-87. The compound or pharmaceutically acceptable salt thereof according to embodiment A-86, wherein the compound of formula (A-IV-A-1-a-i) is a compound of formula (A-IV-A-1-a-iii) or (A-IV-A-1-a-iv):

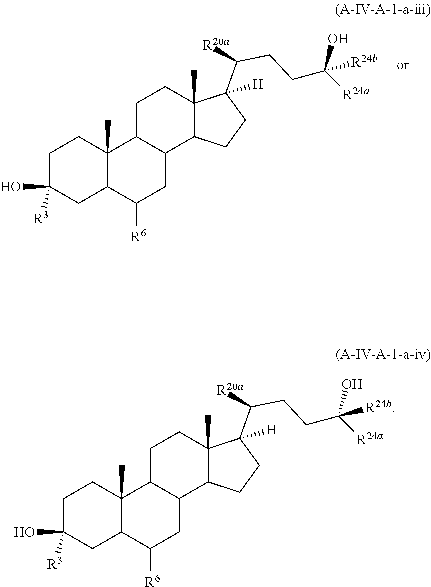

A-88. The compound or pharmaceutically acceptable salt thereof according to embodiment A-86, wherein the compound of formula (A-IV-A-1-a-ii) is a compound of formula (A-IV-A-1-a-v) or (A-IV-A-1-a-vi):

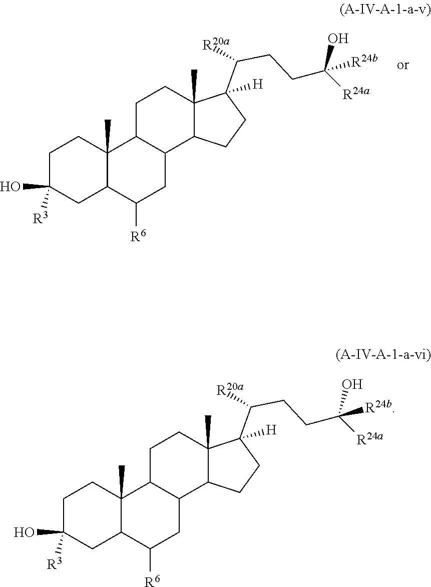

A-89. The compound or pharmaceutically acceptable salt thereof according to embodiment A-84, wherein the compound of formula (A-IV-A-2) is a compound of formula (A-IV-A-2-a):

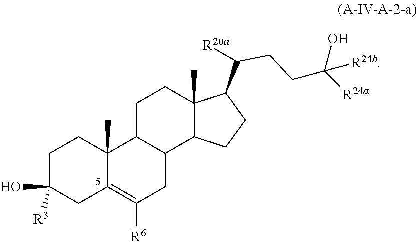

A-90. The compound or pharmaceutically acceptable salt thereof according to embodiment A-89, wherein the compound of formula (A-IV-A-2-a) is a compound of formula (A-IV-A-2-a-i) or (A-IV-A-2-a-ii):

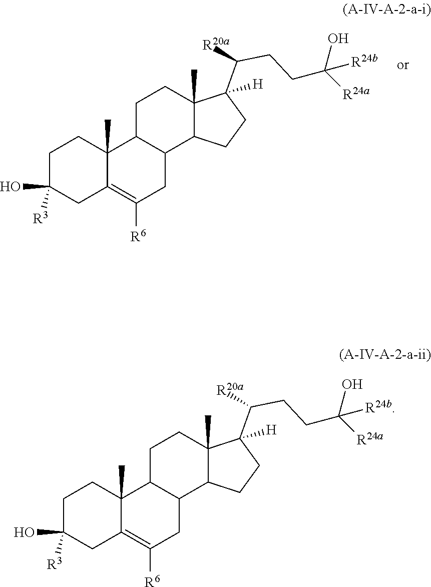

A-91. The compound or pharmaceutically acceptable salt thereof according to embodiment A-90, wherein the compound of formula (A-IV-A-2-a-i) is a compound of formula (A-IV-A-2-a-iii) or (A-IV-A-2-a-iv):

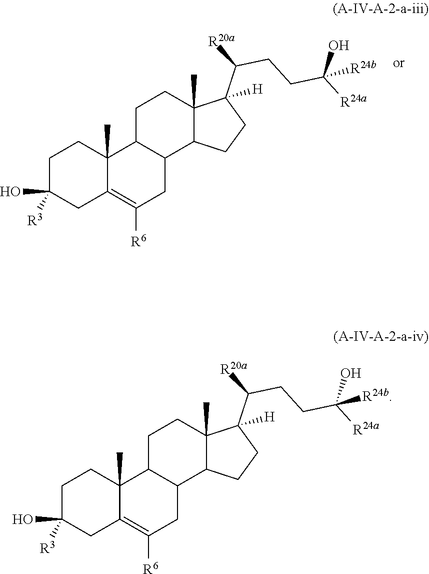

A-92. The compound or pharmaceutically acceptable salt thereof according to embodiment A-90, wherein the compound of formula (A-IV-A-2-a-ii) is a compound of formula (A-IV-A-2-a-v) or (A-IV-A-2-a-vi):

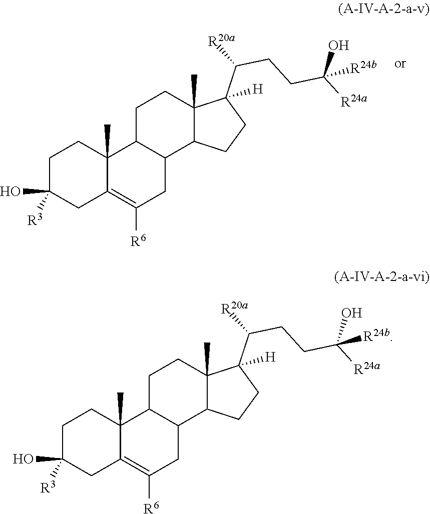

A-93. The compound or pharmaceutically acceptable salt thereof according to embodiment A-83, wherein the compound of formula (A-IV-B) is a compound of formula (A-IV-B-1) or (A-IV-B-2):

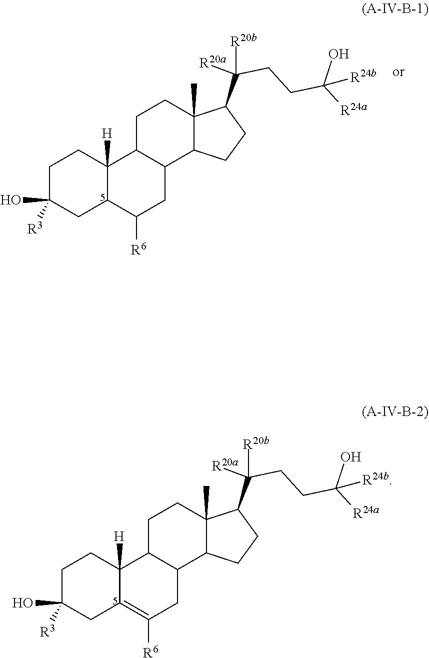

A-94. The compound or pharmaceutically acceptable salt thereof according to embodiment A-93, wherein the compound of formula (A-IV-B-1) is a compound of formula (A-IV-B-1-a):

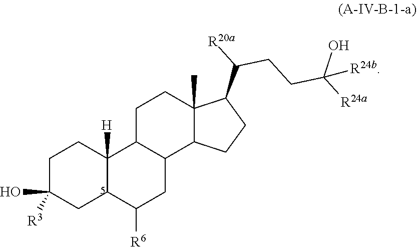

A-95. The compound or pharmaceutically acceptable salt thereof according to embodiment A-94, wherein the compound of formula (A-IV-B-1-a) is a compound of formula (A-IV-B-1-a-i) or (A-IV-B-1-a-ii):

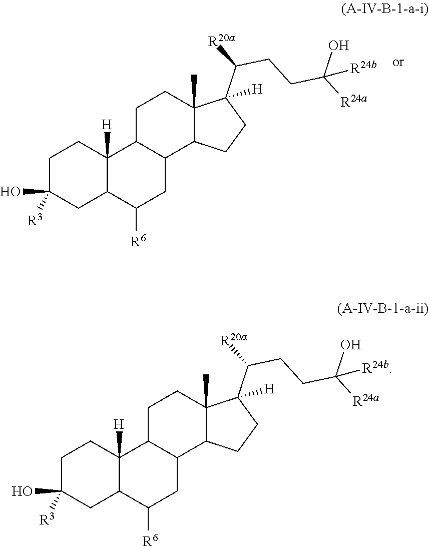

A-96. The compound or pharmaceutically acceptable salt thereof according to embodiment A-95, wherein the compound of formula (A-IV-B-1-a-i) is a compound of formula (A-IV-B-1-a-iii) or (A-IV-B-1-a-iv):

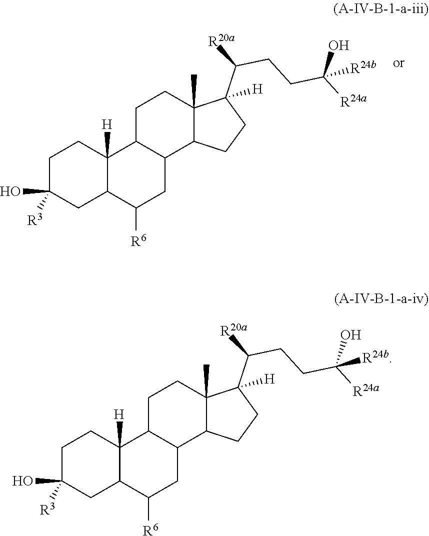

A-97. The compound or pharmaceutically acceptable salt thereof according to embodiment A-95, wherein, the compound of formula (A-IV-B-1-a-ii) is a compound of formula (A-IV-B-1-a-v) or (A-IV-B-1-a-vi):

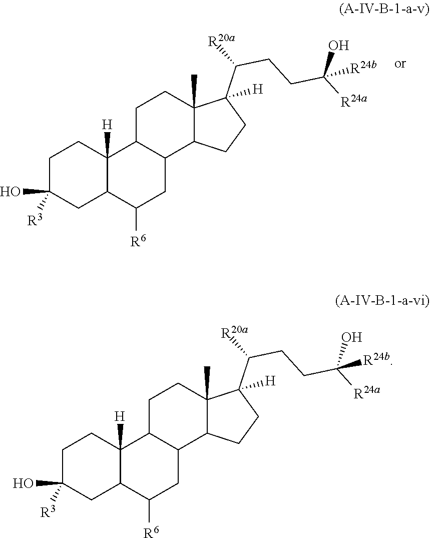

A-98. The compound or pharmaceutically acceptable salt thereof according to embodiment A-93, wherein the compound of formula (A-IV-B-2) is a compound of formula (A-IV-B-2-a):

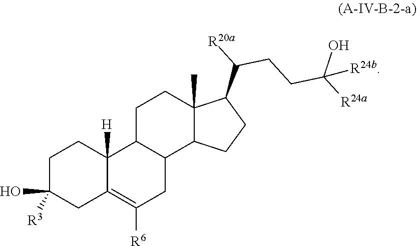

A-99. The compound or pharmaceutically acceptable salt thereof according to embodiment A-98, wherein the compound of formula (A-IV-B-2-a) is a compound of formula (A-IV-B-2-a-i) or (A-IV-B-2-a-ii):

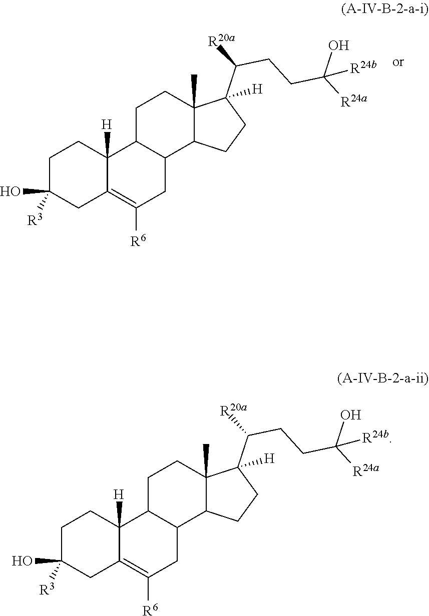

A-100. The compound or pharmaceutically acceptable salt thereof according to embodiment A-99, wherein the compound of formula (A-IV-B-2-a-i) is a compound of formula (A-IV-B-2-a-iii) or (A-IV-B-2-a-iv):

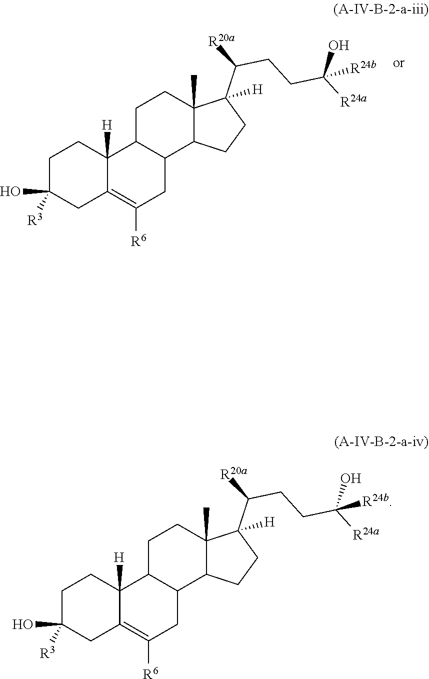

A-101. The compound or pharmaceutically acceptable salt thereof according to embodiment A-99, wherein the compound of formula (A-IV-B-2-a-ii) is a compound of formula (A-IV-B-2-a-v) or (A-IV-B-2-a-vi):

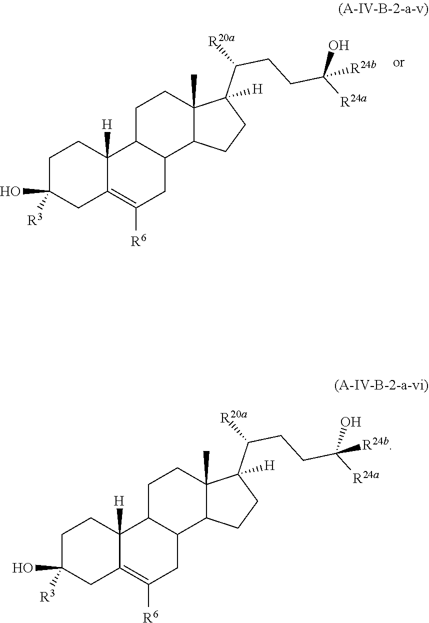

A-102. The compound or pharmaceutically acceptable salt thereof according to embodiment A-82, wherein the compound of formula (A-V) is a compound of formula (A-V-A) or (A-V-B):

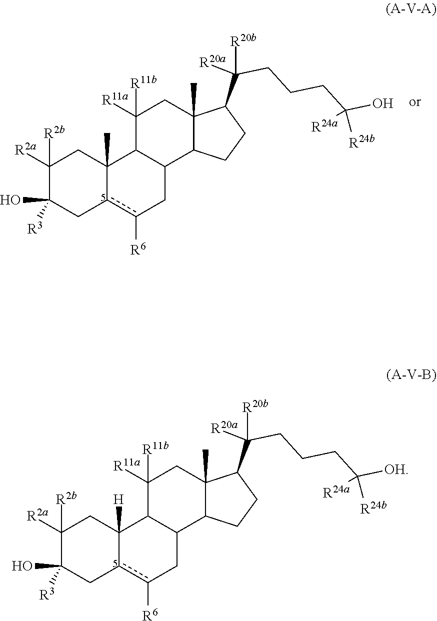

A-103. The compound or pharmaceutically acceptable salt thereof according to embodiment A-102, wherein the compound of formula (A-V-A) is a compound of formula (A-V-A-1) or (A-V-A-2):

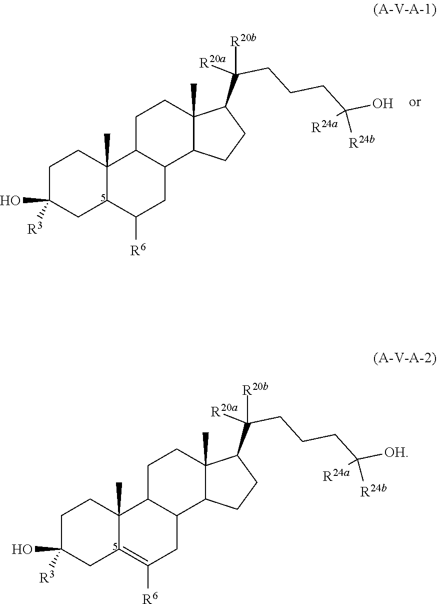

A-104. The compound or pharmaceutically acceptable salt thereof according to embodiment A-103, wherein the compound of formula (A-V-A-1) is a compound of formula (A-V-A-1-a):

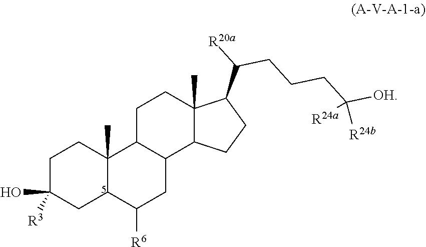

A-105. The compound or pharmaceutically acceptable salt thereof according to embodiment A-104, wherein the compound of formula (A-V-A-1-a) is a compound of formula (A-V-A-1-a-i) or (A-V-A-1-a-ii):

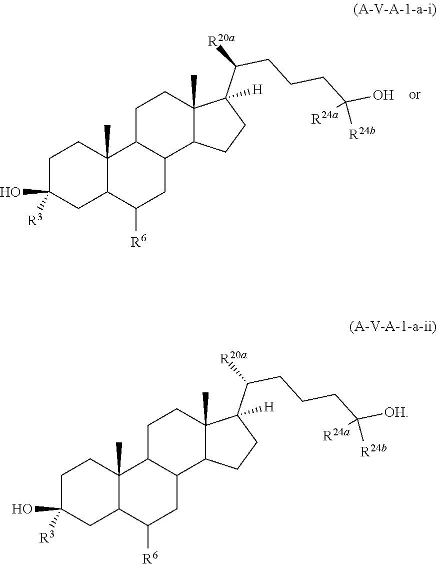

A-106. The compound or pharmaceutically acceptable salt thereof according to embodiment A-105, wherein the compound of formula (A-V-A-1-a-i) is a compound of formula (A-V-A-1-a-iii) or (A-V-A-1-a-iv):

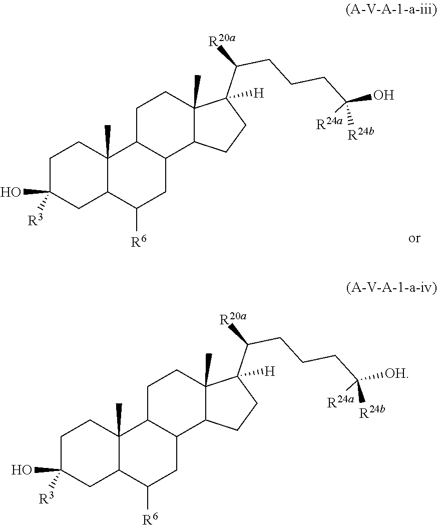

A-107. The compound of pharmaceutically acceptable salt thereof according to embodiment A-105, wherein the compound of formula (A-V-A-1-a-ii) is a compound of formula (A-V-A-1-a-v) or (A-V-A-1-a-vi):

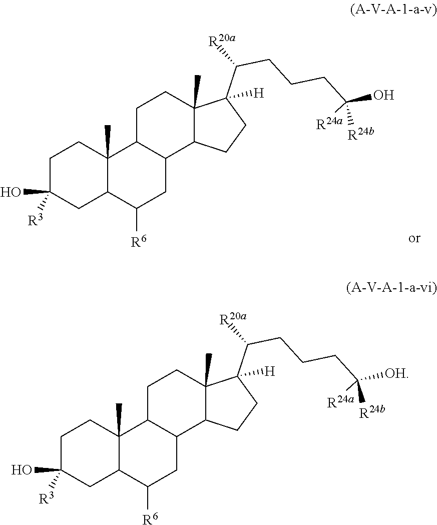

A-108. The compound or pharmaceutically acceptable salt thereof according to embodiment A-103, wherein the compound of formula (A-V-A-2) is a compound of formula (A-V-A-2-a):

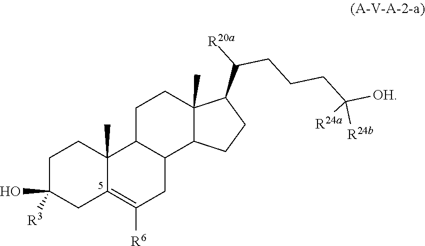

A-109. The compound or pharmaceutically acceptable salt thereof according to embodiment A-108, wherein the compound of formula (A-V-A-2-a) is a compound of formula (A-V-A-2-a-i) or (A-V-A-2-a-ii):

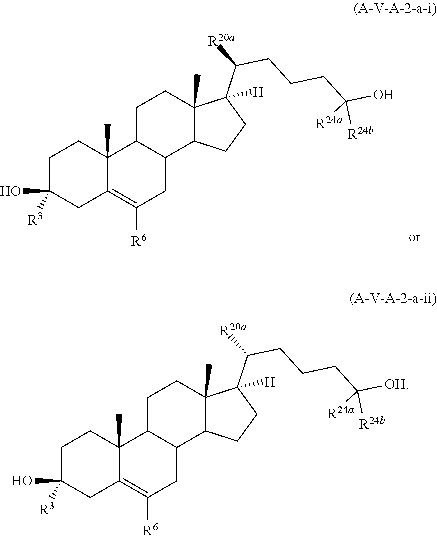

A-110. The compound or pharmaceutically acceptable salt thereof according to embodiment A-109, wherein the compound of formula (A-V-A-2-a-i) is a compound of formula (A-V-A-2-a-iii) or (A-V-A-2-a-iv):

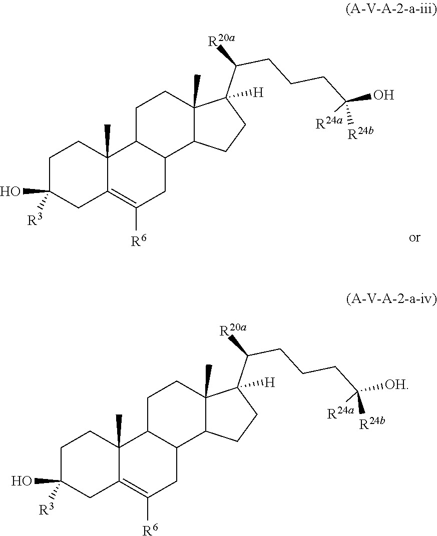

A-111. The compound or pharmaceutically acceptable salt thereof according to embodiment A-109, wherein the compound of formula (A-V-A-2-a-ii) is a compound of formula (A-V-A-2-a-v) or (A-V-A-2-a-vi):

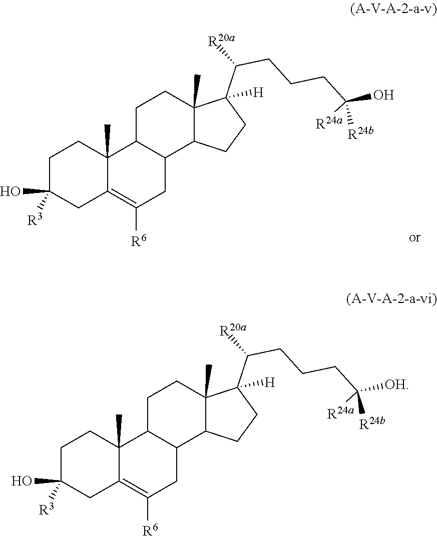

A-112. The compound or pharmaceutically acceptable salt thereof according to embodiment A-102, wherein the compound of formula (A-V-B) is a compound of formula (A-V-B-1) or (A-V-B-2):

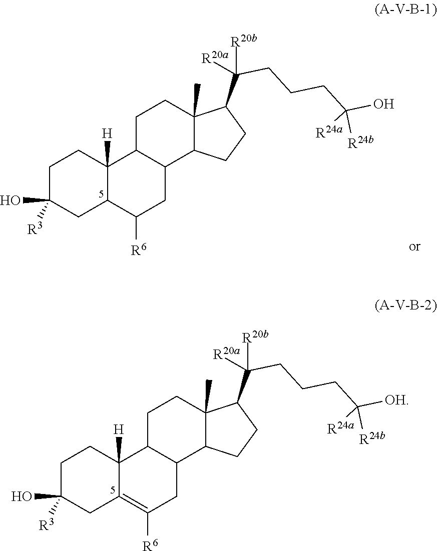

A-113. The compound of pharmaceutically acceptable salt thereof according to embodiment A-112, wherein the compound of formula (A-V-B-1) is a compound of formula (A-V-B-1-a):

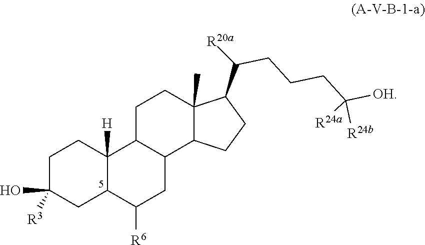

A-114. The compound or pharmaceutically acceptable salt thereof according to embodiment A-113, wherein the compound of formula (A-V-B-1-a) is a compound of formula (A-V-B-1-a-i) or (A-V-B-1-a-ii):

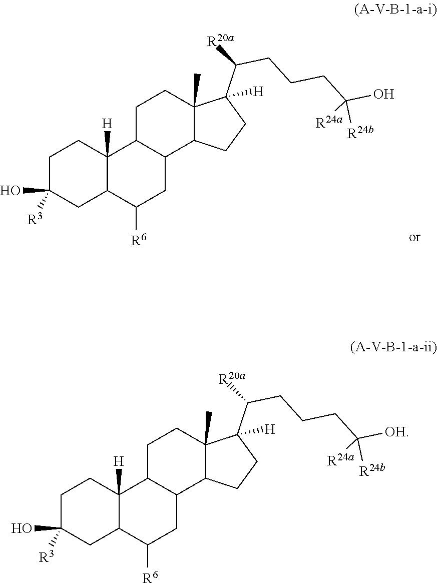

A-115. The compound or pharmaceutically acceptable salt thereof according to embodiment A-114, wherein the compound of formula (A-V-B-1-a-i) is a compound of formula (A-V-B-1-a-iii) or (A-V-B-1-a-iv):

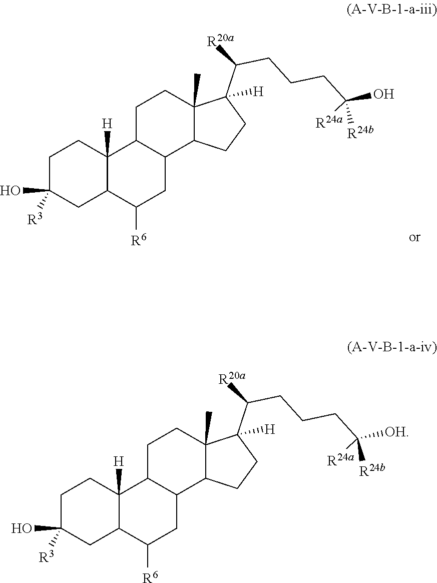

A-116. The compound or pharmaceutically acceptable salt thereof according to embodiment A-114, wherein the compound of formula (A-V-B-1-a-ii) is a compound of formula (A-V-B-1-a-v) or (A-V-B-1-a-vi):

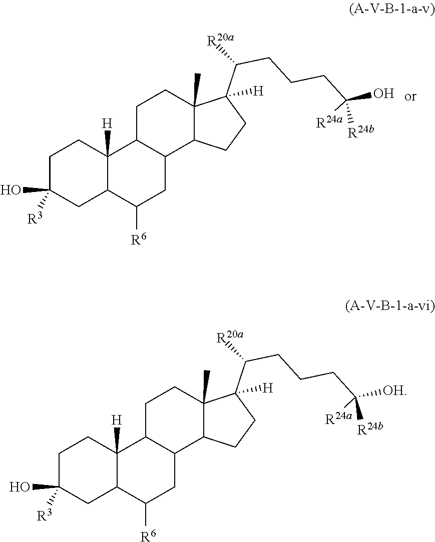

A-117. The compound or pharmaceutically acceptable salt thereof according to embodiment A-112, wherein the compound of formula (A-V-B-2) is a compound of formula (A-V-B-2-a):

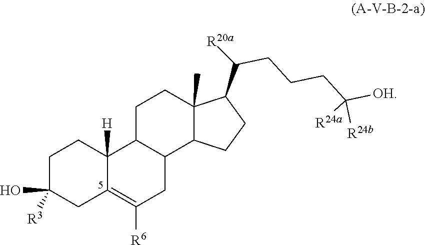

A-118. The compound or pharmaceutically acceptable salt thereof according to embodiment A-117, wherein the compound of formula (A-V-B-2-a) is a compound of formula (A-V-B-2-a-i) or (A-V-B-2-a-ii):

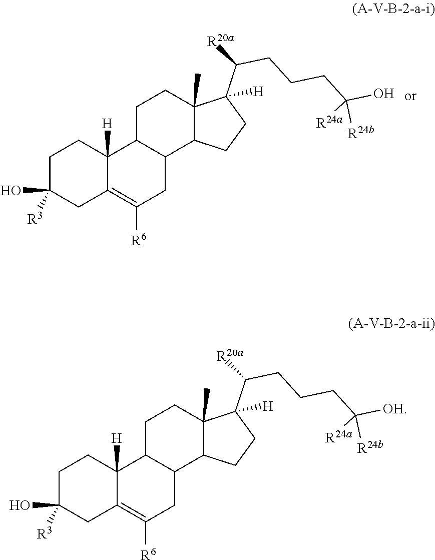

A-119. The compound or pharmaceutically acceptable salt thereof according to embodiment A-118, wherein the compound of formula (A-V-B-2-a-i) is a compound of formula (A-V-B-2-a-iii) or (A-V-B-2-a-iv):

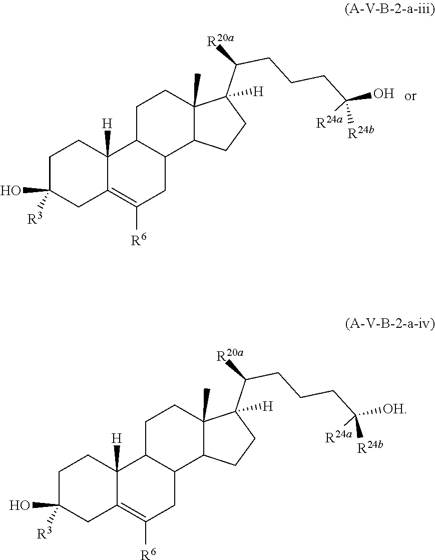

A-120. The compound or pharmaceutically acceptable salt thereof according to embodiment A-118, wherein the compound of formula (A-V-B-2-a-ii) is a compound of formula (A-V-B-2-a-v) or (A-V-B-2-a-vi):

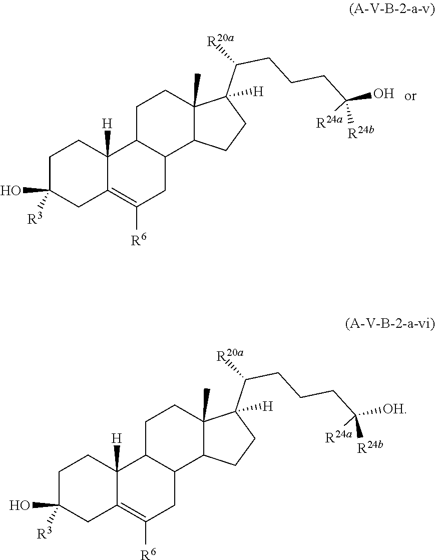

A-121. The compound or pharmaceutically acceptable salt thereof according to embodiment A-1, wherein the compound of formula (A-I) is a compound of formula (A-VI):

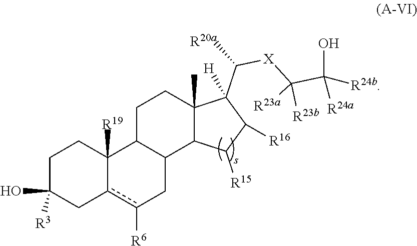

A-122. The compound or pharmaceutically acceptable salt thereof according to embodiment A-1, wherein the compound of formula (A-I) is a compound of formula (A-VII):

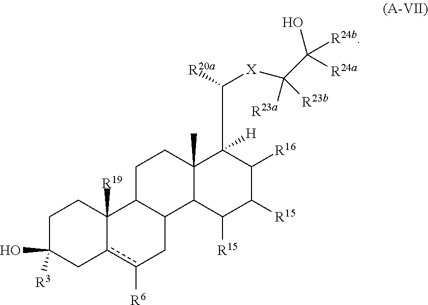

A-123. The compound or pharmaceutically acceptable salt thereof according to embodiment A-1, wherein the compound of formula (A-I) is a compound of formula (A-VIII):

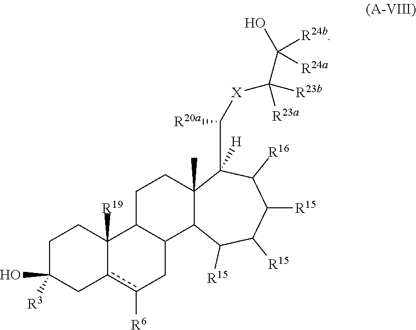

A-124. The compound or pharmaceutically acceptable salt thereof according to embodiment A-1, wherein the compound of formula (A-I) is a compound of formula (A-IX):

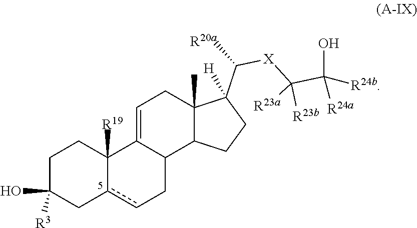

A-125. The compound or pharmaceutically acceptable salt thereof according to embodiment A-1, wherein the compound of formula (A-I) is a compound of formula (A-X):

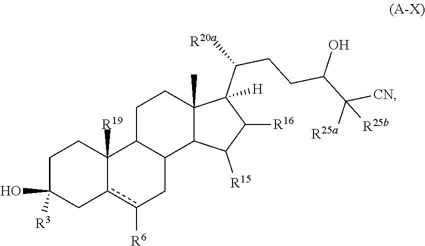

wherein:
R^{25a} is selected from the group consisting of substituted or unsubstituted C₁₋₆ alkyl, substituted or unsubstituted C₂₋₆alkenyl, substituted or unsubstituted C₂₋₆ alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl;
R^{25b} is hydrogen, substituted or unsubstituted C₁₋₆alkyl group, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, or substituted or unsubstituted 5- to 10-membered heteroaryl; or
R^{25a} and R^{25b}, taken together with the carbon atom to which they are attached, form a substituted or unsubstituted C₃₋₆carbocyclyl; and
R³, R⁶, R¹⁵, R¹⁶, R¹⁹, and R^{20a} are as defined in embodiment A-1.

A-126. The compound or pharmaceutically acceptable salt thereof according to embodiment A-1, wherein the compound of formula (A-I) is a compound of formula (A-XI):

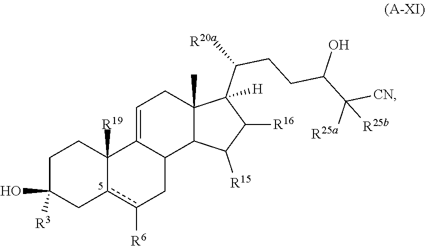

wherein:
R^{25a} is selected from the group consisting of substituted or unsubstituted C₁₋₆ alkyl, substituted or unsubstituted C₂₋₆alkenyl, substituted or unsubstituted C₂₋₆ alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl;
R^{25b} is hydrogen, substituted or unsubstituted C₁₋₆alkyl group, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, or substituted or unsubstituted 5- to 10-membered heteroaryl; or
R^{25a} and R^{25b}, taken together with the carbon atom to which they are attached, form a substituted or unsubstituted C₃₋₆carbocyclyl; and
R³, R⁶, R¹⁵, R¹⁶, R¹⁹, and R^{20a} are as defined in embodiment A-1.

A-127. The compound or pharmaceutically acceptable salt thereof according to embodiment A-1, wherein the compound of formula (A-I) is a compound of formula (A-XII):

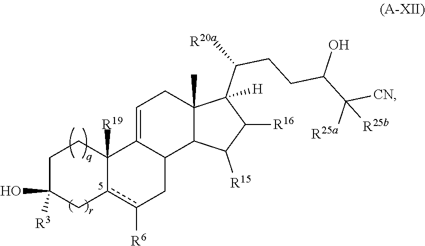

wherein:
R^{25a} is selected from the group consisting of substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl;
R^{25b} is hydrogen, substituted or unsubstituted C₁₋₆alkyl group, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, or substituted or unsubstituted 5- to 10-membered heteroaryl; or
R^{25a} and R^{25b}, taken together with the carbon atom to which they are attached, form a substituted or unsubstituted C₃₋₆carbocyclyl; and
R³, R⁶, R¹⁵, R¹⁶, R¹⁹, and R^{20a} are as defined in embodiment A-1.

A-128. The compound or pharmaceutically acceptable salt thereof according to embodiment A-127, wherein the compound of formula (A-XII) is a compound of formula (A-XII-A) or (A-XII-B):

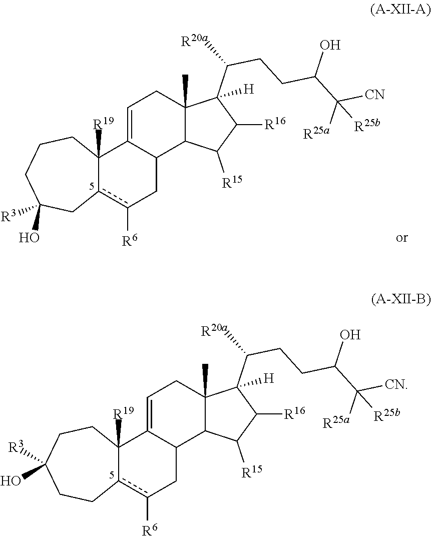

A-129. The compound or pharmaceutically acceptable salt thereof according to embodiment A-1, wherein the compound of formula (A-I) is a compound of formula (A-XIII):

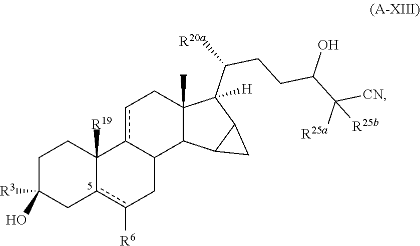

wherein:
R^{25a} is selected from the group consisting of substituted or unsubstituted C₁₋₆ alkyl, substituted or unsubstituted C₂₋₆alkenyl, substituted or unsubstituted C₂₋₆ alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl;
R^{25b} is hydrogen, substituted or unsubstituted C₁₋₆alkyl group, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, or substituted or unsubstituted 5- to 10-membered heteroaryl; or
R^{25a} and R^{25b}, taken together with the carbon atom to which they are attached, form a substituted or unsubstituted C₃₋₆carbocyclyl; and
R³, R⁶, R¹⁹, and R^{20a} are as defined in embodiment A-1.

A-130. The compound or pharmaceutically acceptable salt thereof according to any one of embodiments A-1 to A-4, A-23, A-42 to A-44, A-63, A-82 to A-83, and A-102, wherein each of R^{2a} and R^{2b} is independently hydrogen.

A-131. The compound or pharmaceutically acceptable salt thereof according to any one of embodiments A-1 to A-4, A-23, A-42 to A-44, A-63, A-82 to A-83, and A-102, wherein each of R^{11a} and R^{11b} is independently hydrogen.

A-132. The compound or pharmaceutically acceptable salt thereof according to any one of embodiments A-1 to A-129, wherein R³ is unsubstituted C₁₋₆alkyl.

A-133. The compound or pharmaceutically acceptable salt thereof according to embodiment A-130, wherein R³ is —CH₃, —CH₂CH₃, or —CH₂CH₂CH₃.

A-134. The compound or pharmaceutically acceptable salt thereof according to embodiment A-131, wherein R³ is —CH₂CH₃.

A-135. The compound or pharmaceutically acceptable salt thereof according to any one of embodiments A-1 to A-129, wherein R⁶ is hydrogen.

A-136. The compound or pharmaceutically acceptable salt thereof according to any one of embodiments A-1 to A-129, wherein R^{20a} is halogen, cyano, substituted C₁₋₆ alkyl, unsubstituted C₂₋₆alkyl, or substituted or unsubstituted C₁₋₆alkoxy.

A-137. The compound or pharmaceutically acceptable salt thereof according to embodiment A-136, wherein R^{20a} is halogen.

A-138. The compound or pharmaceutically acceptable salt thereof according to embodiment A-137, wherein R^{20a} is —F.

A-139. The compound or pharmaceutically acceptable salt thereof according to embodiment A-136, wherein R^{20a} is substituted C₁₋₆alkyl.

A-140. The compound or pharmaceutically acceptable salt thereof according to embodiment A-139, wherein R^{20a} is —CF₃.

A-141. The compound or pharmaceutically acceptable salt thereof according to embodiment A-136, wherein R^{20a} is unsubstituted C₂₋₆alkyl.

A-142. The compound or pharmaceutically acceptable salt thereof according to embodiment A-141, wherein R^{20a} is —CH₂CH₃.

A-143. The compound or pharmaceutically acceptable salt thereof according to embodiment A-136, wherein R^{20a} is unsubstituted C₁₋₆alkoxy.

A-144. The compound or pharmaceutically acceptable salt thereof according to embodiment A-143, wherein R^{20a} is —OCH₃ or —OCH₂CH₃.

A-145. The compound or pharmaceutically acceptable salt thereof according to embodiment A-136, wherein R^{20a} is cyano.

A-146. The compound or pharmaceutically acceptable salt thereof according to any one of embodiments A-1 to A-5, A-23 to A-24, A-33, A-42 to A-45, A-54, A-63 to A-64, A-73, A-82 to A-84, A-102 to A-103, and A-112, wherein R^{20b} is hydrogen, halogen, or substituted or unsubstituted alkyl C₁₋₆alkyl.

A-147. The compound or pharmaceutically acceptable salt thereof according to any one of embodiments A-1 to A-2 and A-121 to A-124, wherein X is —(C(R^{X})₂—, wherein

R^{X} is hydrogen or fluorine.

A-148. The compound or pharmaceutically acceptable salt thereof according to embodiment A-147, wherein X is —CH₂—.

A-149. The compound or pharmaceutically acceptable salt thereof according to embodiment A-147, wherein X is —CH₂CH₂—.

A-150. The compound or pharmaceutically acceptable salt thereof according to any one of embodiments A-1 to A-2 and A-121 to A-124, wherein each of R^{23a} and R^{23b} is hydrogen.

A-151. The compound or pharmaceutically acceptable salt thereof according to any one of embodiments A-1 to A-2 and A-121 to A-124, wherein each of R^{23a} and R^{23b} is fluorine.

A-152. The compound or pharmaceutically acceptable salt thereof according to any one of embodiments A-1 to A-129, wherein R^{24a} is substituted or unsubstituted C₁₋₆alkyl.

A-153. The compound or pharmaceutically acceptable salt thereof according to embodiment A-152, wherein R^{24a} is —CH₃, —CH(CH₃)₂, or —CF₃.

A-154. The compound or pharmaceutically acceptable salt thereof according to any one of embodiments A-1 to A-129, wherein R^{24b} is hydrogen or unsubstituted C₁₋₆alkyl.

A-155. The compound or pharmaceutically acceptable salt thereof according to embodiment A-154, wherein R^{24b} is hydrogen.

A-156. The compound or pharmaceutically acceptable salt thereof according to embodiment A-154, wherein R^{24b} is —CH₃.

A-157. The compound or pharmaceutically acceptable salt thereof according to any one of embodiments A-1 to A-9, A-14 to A-18, A-23 to A-28, A-33 to A-37, A-42 to A-49, A-54 to A-58, A-63 to A-68, A-73 to A-77, A-82 to A-88, A-93 to A-97, A-102 to A-107, A-112 to A-117, A-121 to A-129, wherein the bond between C5 and C6 is a single bond and R⁵ is hydrogen in the alpha configuration.

A-158. The compound or pharmaceutically acceptable salt thereof according to embodiment A-1, wherein the bond between C9 and C11 is a double bond, R⁹ is absent and R^{11a} is selected from the group consisting of hydrogen, halogen, substituted or unsubstituted C₁₋₆alkyl, and —OR^{A11}, wherein R^{A11} is hydrogen or substituted or unsubstituted C₁₋₆alkyl, and R^{11b} is absent or hydrogen.

A-159. The compound or pharmaceutically acceptable salt thereof according to embodiment A-1, wherein the bond between C9 and C11 is a single bond, R⁹ is hydrogen and R¹¹ is selected from the group consisting of halogen or substituted or unsubstituted C₁₋₆alkyl.

A-160. The compound or pharmaceutically acceptable salt thereof according to any one of embodiments A-1, A-121 to A-123, and A-125 to A-128, wherein each instance of R¹⁵ is independently hydrogen, substituted or unsubstituted C₁₋₆alkyl or substituted or unsubstituted C₃₋₆carbocyclyl,

A-161. The compound or pharmaceutically acceptable salt thereof according to embodiment A-160, wherein each instance of R¹⁵ is independently hydrogen, unsubstituted C₁₋₆alkyl or unsubstituted C₃₋₆carbocyclyl.

A-162. The compound or pharmaceutically acceptable salt thereof according to embodiment A-161, wherein each instance of R¹⁵ is independently hydrogen, —CH₃ or cyclopropyl.

A-163. The compound or pharmaceutically acceptable salt thereof according to embodiment A-162, wherein each instance of R¹⁵ is independently hydrogen.

A-164. The compound or pharmaceutically acceptable salt thereof according to embodiment A-162, wherein each instance of R¹⁵ is independently —CH₃.

A-165. The compound or pharmaceutically acceptable salt thereof according to embodiment A-162, wherein each instance of R¹⁵ is independently cyclopropyl.

A-166. The compound or pharmaceutically acceptable salt thereof according to any one of embodiments A-1, A-121 to A-123, and A-125 to A-128, wherein R¹⁶ is hydrogen, substituted or unsubstituted C₁₋₆alkyl or substituted or unsubstituted C₃₋₆ carbocyclyl.

A-167. The compound or pharmaceutically acceptable salt thereof according to embodiment A-166, wherein R¹⁶ is hydrogen or unsubstituted C₁₋₆alkyl.

A-168. The compound or pharmaceutically acceptable salt thereof according to embodiment A-166, wherein R¹⁶ is hydrogen.

A-169. The compound or pharmaceutically acceptable salt thereof according to embodiment A-166, wherein R¹⁶ is —CH₃.

A-170. The compound or pharmaceutically acceptable salt thereof according to any one of embodiments A-1, A-121 to A-123, and A-125 to A-128, wherein R¹⁵ and R¹⁶ are taken together to form a substituted or unsubstituted C₃₋₆carbocyclyl.

A-171. The compound or pharmaceutically acceptable salt thereof according to embodiment A-170, wherein R¹⁵ and R¹⁶ are taken together to form a cyclopropyl.

A-172. The compound or pharmaceutically acceptable salt thereof according to embodiment A-1, wherein R¹⁸ is hydrogen, unsubstituted C₁₋₆alkyl or C₁₋₆alkyl substituted with OR^{A18}, wherein R^{A18} is substituted or unsubstituted C₁₋₆alkyl.

A-173. The compound or pharmaceutically acceptable salt thereof according to embodiment A-1, wherein R¹⁸ is hydrogen, —CH₃, —CH₂OCH₃, —OCH₃, —CH₂CH₃, or —CH(CH₃)₂.

A-174. The compound or pharmaceutically acceptable salt thereof according to embodiment 1, wherein R¹⁸ is hydrogen or —CH₃.

A-175. The compound or pharmaceutically acceptable salt thereof according to embodiment A-1, wherein R¹⁸ is —CH₃.

A-176. The compound or pharmaceutically acceptable salt thereof according to embodiment A-1, wherein R¹⁸ is hydrogen.

A-177. The compound or pharmaceutically acceptable salt thereof according to any one of embodiments A-1 to A-3, A-42 to A-43, A-82, and A-121 to A-129, wherein R¹⁹ is hydrogen, unsubstituted C₁₋₆alkyl or C₁₋₆alkyl substituted with OR^{A19}, wherein R^{A19} is substituted or unsubstituted C₁₋₆alkyl.

A-178. The compound or pharmaceutically acceptable salt thereof according to any one of embodiments A-1 to A-3, A-42 to A-43, A-82, and A-121 to A-129, wherein R¹⁹ is hydrogen, —CH₃, CH₂OCH₃, —OCH₃, —CH₂CH₃, or —CH(CH₃)₂.

A-179. The compound or pharmaceutically acceptable salt thereof according to any one of embodiments A-1 to A-3, A-42 to A-43, A-82, and A-121 to A-129, wherein R¹⁹ is hydrogen or —CH₃.

A-180. The compound or pharmaceutically acceptable salt thereof according to any one of embodiments A-1 to A-3, A-42 to A-43, A-82, and A-121 to A-129, wherein R¹⁹ is —CH₃.

A-181. The compound or pharmaceutically acceptable salt thereof according to any one of embodiments A-1 to A-3, A-42 to A-43, A-82, and A-121 to A-129, wherein R¹⁹ is hydrogen.

A-182. The compound or pharmaceutically acceptable salt thereof according to embodiment A-1, wherein the compound of Formula (A-I) is selected from the group consisting of compounds A-1-A-26 of Table A-1 and pharmaceutically acceptable salts thereof.

A-183. The compound or pharmaceutically acceptable salt thereof according to embodiment A-1, wherein the compound of Formula (A-I) is selected from the group consisting of compounds A-1-A-22 of Table A-1 and pharmaceutically acceptable salts thereof.

A-184. The compound or pharmaceutically acceptable salt thereof according to embodiment A-1, wherein the compound of Formula (A-I) is selected from the group consisting of compounds A-3, A-4, A-5, A-6, A-7, A-9, A-13, A-14, A-15, A-16 of Table A-1 and pharmaceutically acceptable salts thereof.

A-185. A pharmaceutical composition comprising a compound or pharmaceutically acceptable salt thereof according to any one of embodiments A-1 to A-184, and a pharmaceutically acceptable carrier.

A-186. A method for treating a CNS-related condition in a subject, comprising administering to the subject an effective amount of a compound or pharmaceutically acceptable salt thereof according to any one of embodiments A-1 to A-184, or a pharmaceutical composition according to embodiment A-185.

A-187. The method according to embodiment A-186, wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease) and tinnitus.

A-188. A method of inducing sedation or anesthesia in a subject, comprising administering to the subject an effective amount of a compound or pharmaceutically acceptable salt thereof according to any one of embodiments A-1 to A-184, or a pharmaceutical composition according to embodiment A-185.

A-189. A compound or pharmaceutically acceptable salt thereof according to any one of embodiments A-1 to A-184, or a pharmaceutical composition according to embodiment A-185, for use in treating a CNS-related condition in a subject.

A-190. The compound, or pharmaceutically acceptable salt thereof, or pharmaceutical composition for use according to embodiment A-189, wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease) and tinnitus.

A-191. A compound or pharmaceutically acceptable salt thereof according to any one of embodiments A-1 to A-184, or a pharmaceutical composition according to embodiment A-185, for use in inducing sedation or anesthesia in a subject.

A-192. Use of a compound or pharmaceutically acceptable salt thereof according to any one of embodiments A-1 to A-184, or a pharmaceutical composition according to embodiment A-185, for the manufacture of a medicament for treating a CNS-related condition in a subject.

A-193. The use according to embodiment A-192, wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease) and tinnitus.

A-194. Use of a compound or pharmaceutically acceptable salt thereof according to any one of embodiments A-1 to A-184, or a pharmaceutical composition according to embodiment A-185, for the manufacture of a medicament for inducing sedation or anesthesia in a subject.

A-195. A compound of Formula (A-I):

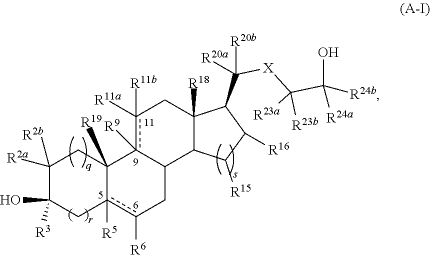

wherein:
   each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein R^{A2} is hydrogen or substituted or unsubstituted C₁₋₆alkyl;
   R³ is substituted or unsubstituted C₁₋₆alkyl, substituted or unsubstituted C₂₋₆alkenyl, substituted or unsubstituted C₂₋₆alkynyl, or substituted or unsubstituted C₃₋₁₀carbocyclyl;
   R⁵ is absent, hydrogen, halogen, or substituted or unsubstituted C₁₋₆alkyl;
   R⁶ is hydrogen, halogen, or substituted or unsubstituted C₁₋₆alkyl;
   R⁹ is absent or hydrogen; or
   R⁹ and R^{11a} are taken together with the carbon atoms to which they are attached to form a substituted or unsubstituted C₃₋₆carbocyclyl ring;
   R^{11a} is hydrogen, halogen, substituted or unsubstituted C₁₋₆alkyl, or —OR^{A11}, wherein R^{A11} is hydrogen or substituted or unsubstituted C₁₋₆alkyl;
   R^{11b} is absent or hydrogen; or
   R^{11a} and R^{11b} are joined to form an oxo (═O) group;
   each instance of R¹⁵ is hydrogen, halogen, —OH, —NH₂, substituted or unsubstituted C₁₋₆alkyl, substituted or unsubstituted C₂₋₆alkenyl, substituted or unsubstituted C₂₋₆alkynyl, substituted or unsubstituted C₁₋₆alkoxy, substituted or unsubstituted C₃₋₆carbocyclyl, or substituted or unsubstituted 3- to 10-membered heterocyclyl;
   R¹⁶ is hydrogen, halogen, —OH, —NH₂, substituted or unsubstituted C₁₋₆alkyl, substituted or unsubstituted C₂₋₆alkenyl, substituted or unsubstituted C₂₋₆alkynyl, substituted or unsubstituted C₁₋₆alkoxy, substituted or unsubstituted C₃₋₆carbocyclyl, or substituted or unsubstituted 3- to 10-membered heterocyclyl; or
   R¹⁵ and R¹⁶ are taken together with the carbon atoms to which they are attached, to form a substituted or unsubstituted C₃₋₆carbocyclyl;
   R¹⁸ is hydrogen, substituted or unsubstituted C₁₋₆alkyl, or OR^{A18}, wherein each instance of R^{A18} is independently substituted or unsubstituted C₁₋₆alkyl;
   R¹⁹ is hydrogen, substituted or unsubstituted C₁₋₆alkyl, or OR^{A19}; wherein each instance of R^{A19} is independently substituted or unsubstituted C₁₋₆alkyl;
   R^{20a} is halogen, cyano, substituted C₁₋₆alkyl, unsubstituted C₂₋₆alkyl, substituted or unsubstituted C₁₋₆alkoxy, or substituted or unsubstituted C₃₋₆ carbocyclyl;
   R^{20b} is hydrogen, halogen, cyano, substituted or unsubstituted C₁₋₆alkyl, substituted or unsubstituted C₁₋₆alkoxy, or substituted or unsubstituted C₃₋₆ carbocyclyl;
   or R^{20a} and R^{20b} are both simultaneously —CH₃;
   X is —(C(R^{X})₂)ₙ— or —O—, wherein R^{X} is hydrogen or fluorine, or one R^{X} group and R^{23b} are joined to form a double bond;
   n is 1, 2 or 3;
   each instance of R^{23a} and R^{23b} is independently hydrogen or fluorine;
   R^{24a} is selected from the group consisting of substituted or unsubstituted C₁₋₆alkyl, substituted or unsubstituted C₂₋₆alkenyl, substituted or unsubstituted C₂₋₆ alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl;
   R^{24b} is hydrogen, substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, or substituted or unsubstituted 5- to 10-membered heteroaryl;
   or R^{24a} and R^{24b}, taken together with the carbon atom to which they are attached, form a substituted or unsubstituted C₃₋₆carbocyclyl or a substituted or unsubstituted 3- to 10-membered heterocyclyl;
   q is 0, 1, or 2;
   r is 1 or 2;
   s is 1, 2, or 3; and
   each [symbol] independently represents a single or double bond, provided if the bond between C5 and C6 is a double bond, R⁵ is absent and if the bond between C9 and C11 is a double bond, R⁹ and R^{11b} are absent.

A-196. The compound according to embodiment A-195, wherein the compound of formula (A-I) is a compound of formula (A-II):

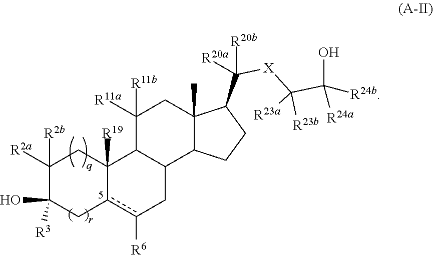

A-197. The compound according to embodiment A-196, wherein the compound of formula (A-II) is a compound of formula (A-II-1) or formula (A-II-2):

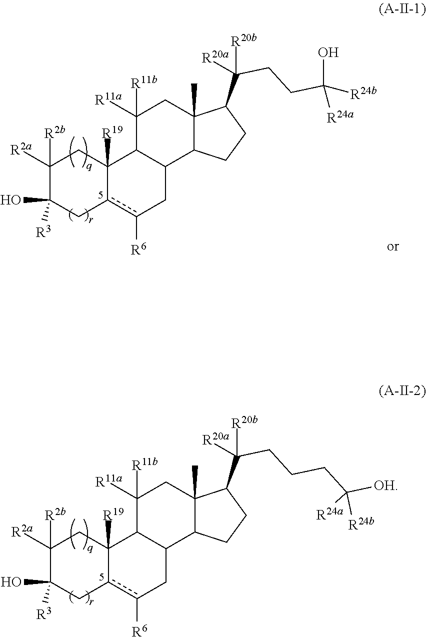

A-198. The compound according to embodiment A-197, wherein the compound of formula (A-II-1) is a compound of formula (A-II-1-A) or formula (A-II-1-B):

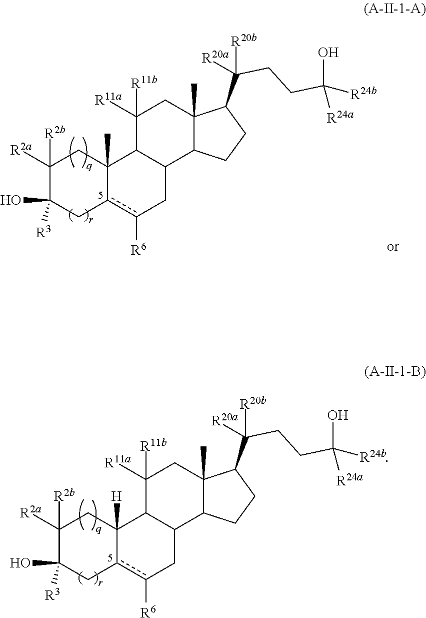

A-199. The compound according to embodiment A-198, wherein the compound of formula (A-II-1-A) is a compound of formula (A-II-1-A-1) or formula (A-II-1-A-2):

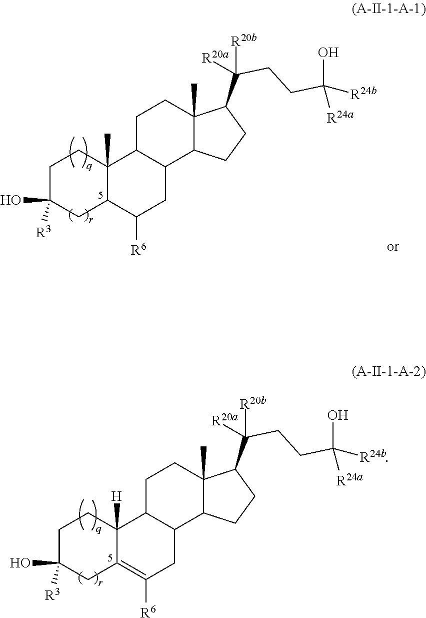

A-200. The compound according to embodiment A-199, wherein the compound of formula (A-II-1-A-1) is a compound of formula (A-II-1-A-1-a):

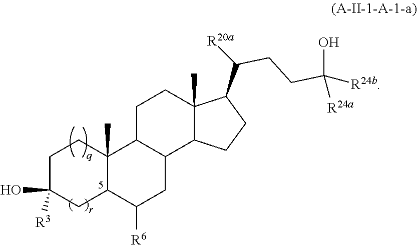

A-201. The compound according to embodiment A-200, wherein the compound of formula (A-II-1-A-1-a) is a compound of formula (A-II-1-A-1-a-i) or (A-II-1-A-1-a-ii):

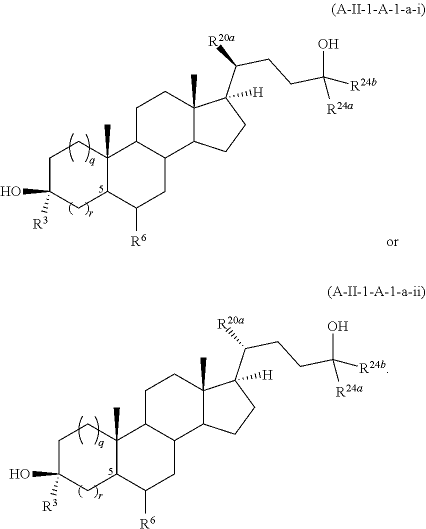

A-202. The compound according to embodiment A-201, wherein the compound of formula (A-II-1-A-1-a-i) is a compound of formula (A-II-1-A-1-a-iii) or (A-II-1-A-1-a-iv):

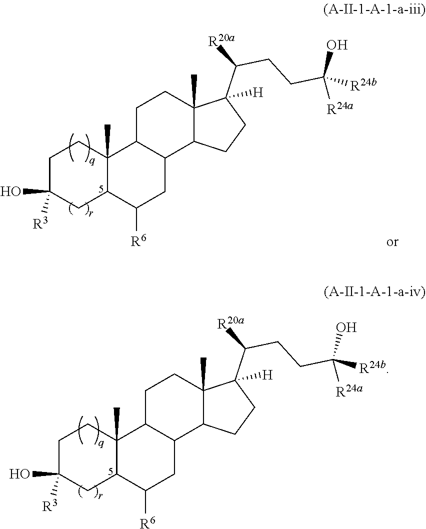

A-203. The compound according to embodiment A-201, wherein the compound of formula (A-II-1-A-1-a-ii) is a compound of formula (A-II-1-A-1-a-v) or (A-II-1-A-1-a-vi):

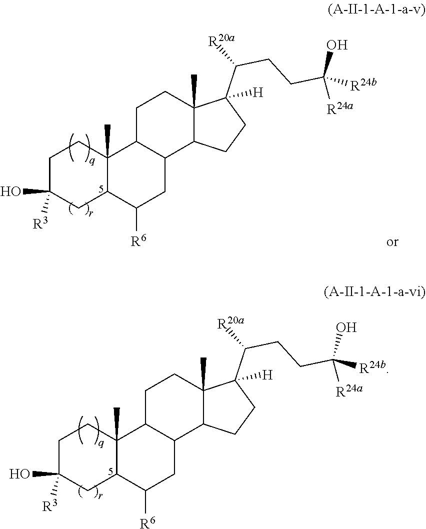

A-204. The compound according to embodiment A-199, wherein the compound of formula (A-II-1-A-2) is a compound of formula (A-II-1-A-2-a):

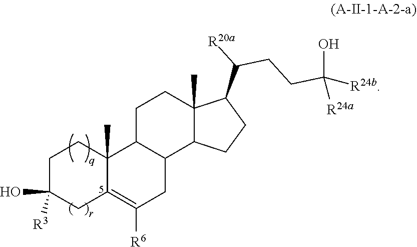

A-205. The compound according to embodiment A-204, wherein the compound of formula (A-II-1-A-2-a) is a compound of formula (A-II-1-A-2-a-i) or (A-II-1-A-2-a-ii):

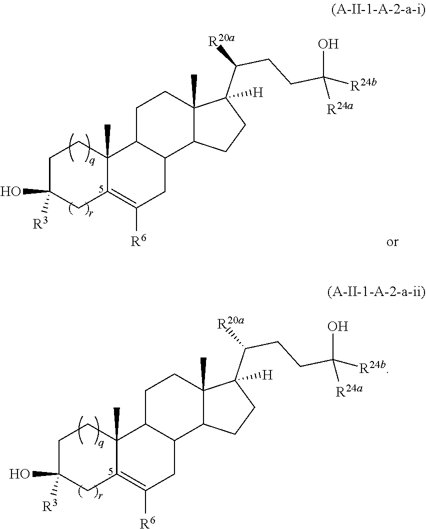

A-206. The compound according to embodiment A-205, wherein the compound of formula (A-II-1-A-2-a-i) is a compound of formula (A-II-1-A-2-a-iii) or (A-II-1-A-2-a-iv):

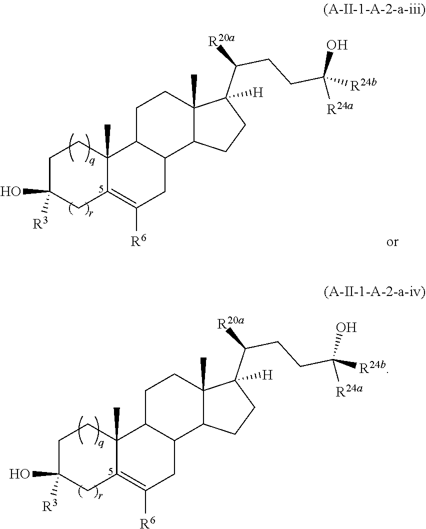

A-207. The compound according to embodiment A-205, wherein the compound of formula (A-II-1-A-2-a-ii) is a compound of formula (A-II-1-A-2-a-v) or (A-II-1-A-2-a-vi):

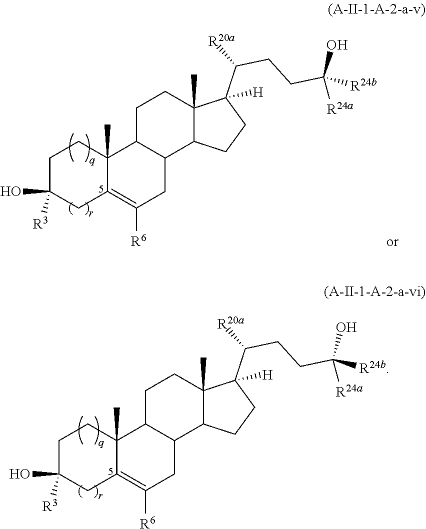

A-208. The compound according to embodiment A-198, wherein the compound of formula (A-II-1-B) is a compound of formula (A-II-1-B-1) or formula (A-II-1-B-2):

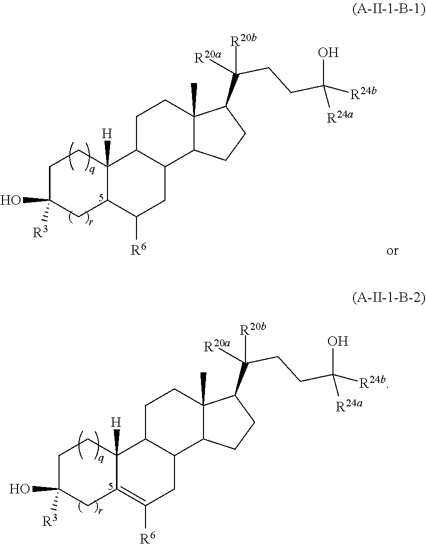

A-209. The compound according to embodiment A-208, wherein the compound of formula (A-II-1-B-1) is a compound of formula (A-II-1-B-1-a):

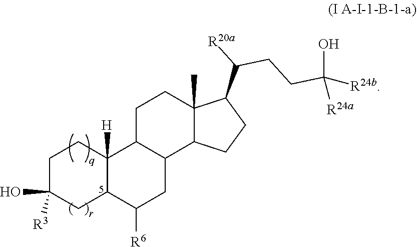

A-210. The compound according to embodiment A-209, wherein the compound of formula (A-II-1-B-1-a) is a compound of formula (A-II-1-B-1-a-i) or formula (A-II-1-B-1-a-ii):

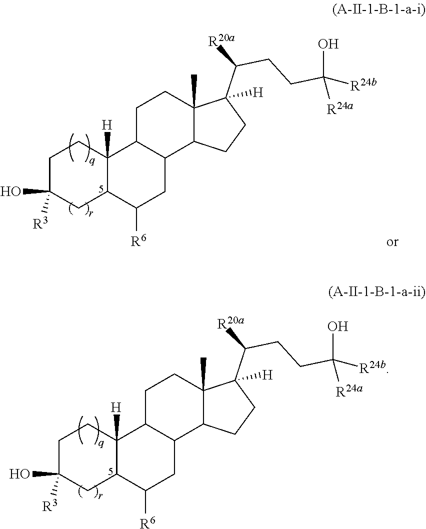

A-211. The compound according to embodiment A-210, wherein the compound of formula (A-II-1-B-1-a-i) is a compound of formula (A-II-1-B-1-a-iii) or formula (A-II-1-B-1-a-iv):

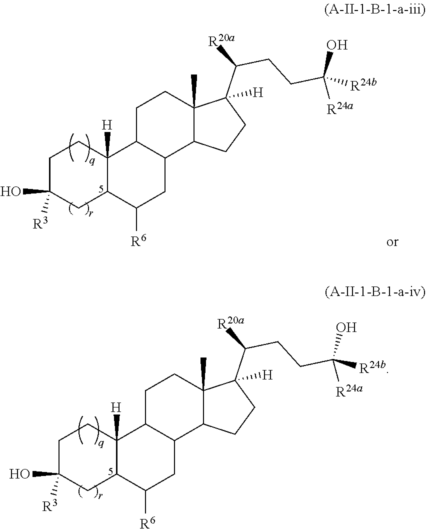

A-212. The compound according to embodiment A-210, wherein the compound of formula (A-II-1-B-1-a-ii) is a compound of formula (A-II-1-B-1-a-v) or formula (A-II-1-B-1-a-vi):

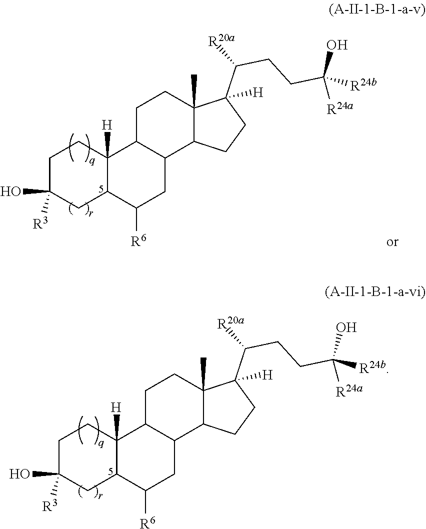

A-213. The compound according to embodiment A-208, wherein the compound of formula (A-II-1-B-2) is a compound of formula (A-II-1-B-2-a):

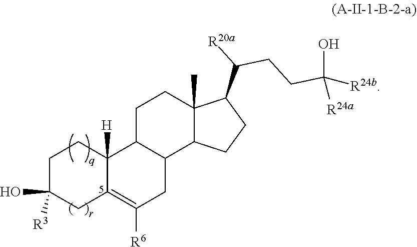

A-214. The compound according to embodiment A-213, wherein the compound of formula (A-II-1-B-2-a) is a compound of formula (A-II-1-B-2-a-i) or formula (A-II-1-B-2-a-ii):

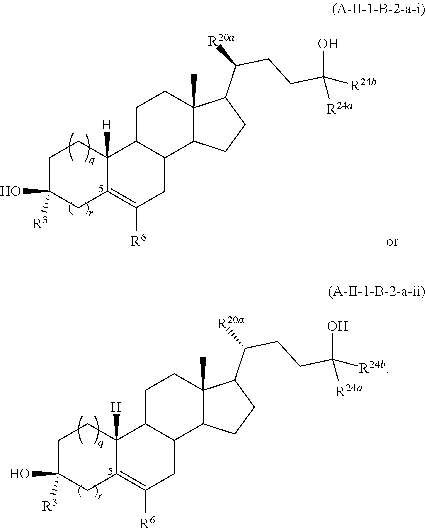

A-215. The compound according to embodiment A-214, wherein the compound of formula (A-II-1-B-2-a-i) is a compound of formula (A-II-1-B-2-a-iii) or formula (A-II-1-B-2-a-iv):

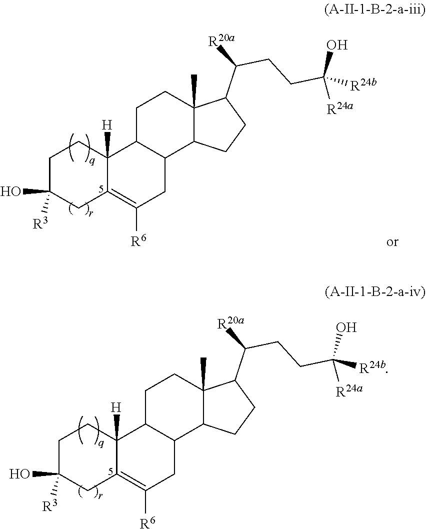

A-216. The compound according to embodiment A-214, wherein the compound of formula (A-II-1-B-2-a-ii) is a compound of formula (A-II-1-B-2-a-v) or formula (A-II-1-B-2-a-vi):

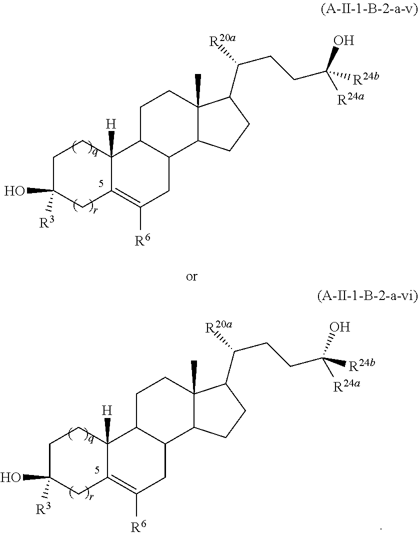

A-217. The compound according to embodiment A-197, wherein the compound of formula (A-II-2) is a compound of formula (A-II-2-A) or formula (A-II-2-B):

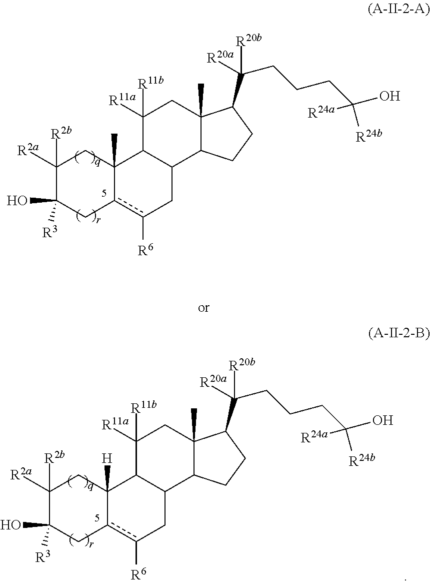

A-218. The compound according to embodiment A-217, wherein the compound of formula (A-II-2-A) is a compound of formula (A-II-2-A-1) or formula (A-II-2-A-2):

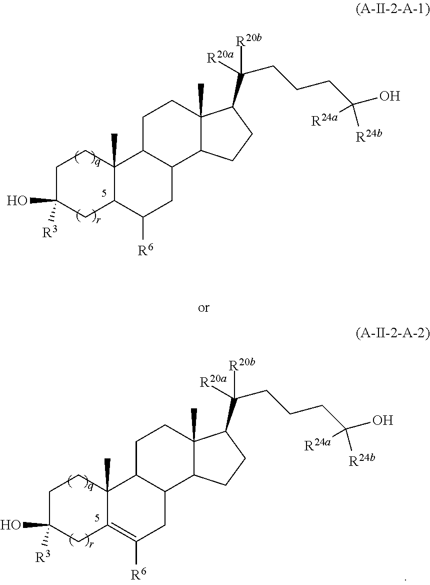

A-219. The compound according to embodiment A-218, wherein the compound of formula (A-II-2-A-1) is a compound of formula (A-II-2-A-1-a):

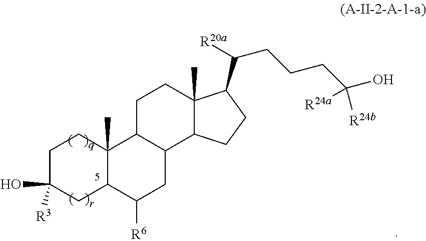

A-220. The compound according to embodiment A-219, wherein the compound of formula (A-II-2-A-1-a) is a compound of formula (A-II-2-A-1-a-i) or formula (A-II-2-A-1-a-ii):

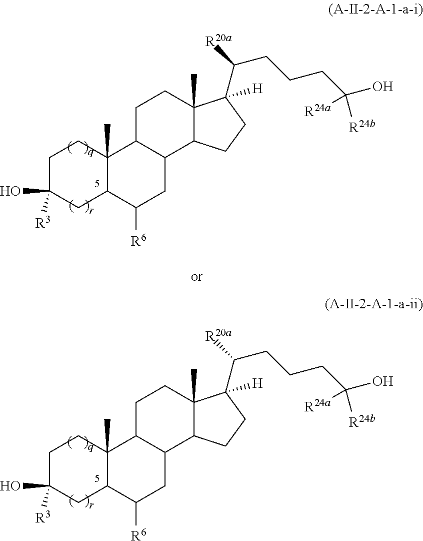

A-221. The compound according to embodiment A-220, wherein the compound of formula (A-II-2-A-1-a-i) is a compound of formula (A-II-2-A-1-a-iii) or formula (A-II-2-A-1-a-iv):

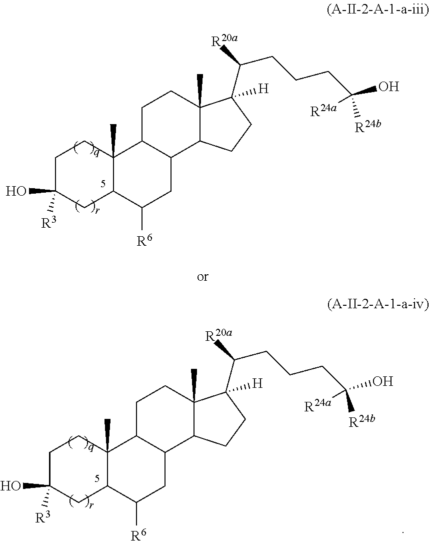

A-222. The compound according to embodiment A-220, wherein the compound of formula (A-II-2-A-1-a-ii) is a compound of formula (A-II-2-A-1-a-v) or formula (A-II-2-A-1-a-vi):

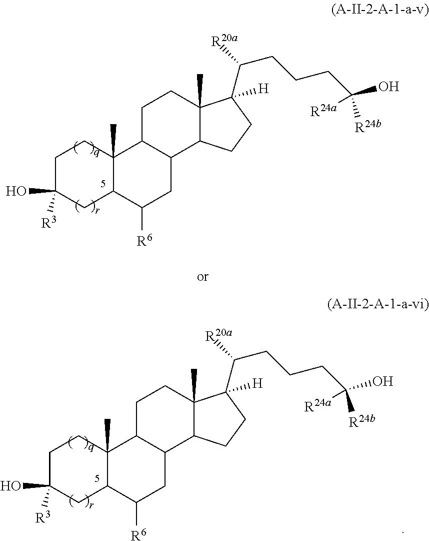

A-223. The compound according to embodiment A-218, wherein the compound of formula (A-II-2-A-2) is a compound of formula (A-II-2-A-2-a):

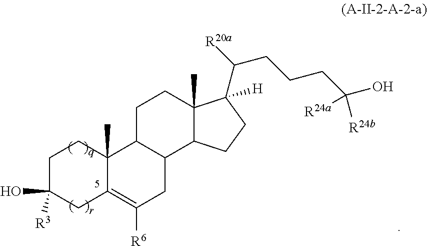

A-224. The compound according to embodiment A-223, wherein the compound of formula (A-II-2-A-2-a) is a compound of formula (A-II-2-A-2-a-i) or formula (A-II-2-A-2-a-ii):

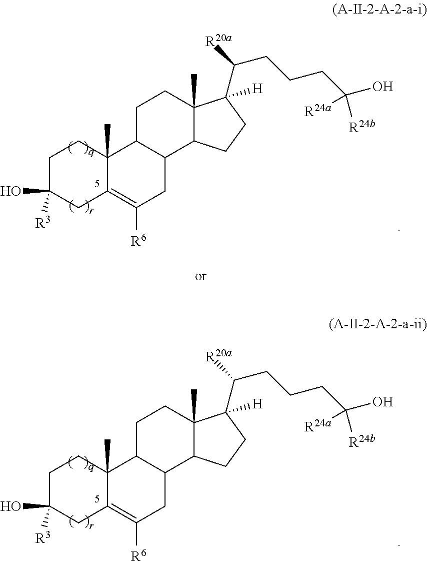

A-225. The compound according to embodiment A-224, wherein the compound of formula (A-II-2-A-2-a-i) is a compound of formula (A-II-2-A-2-a-iii) or formula (A-II-2-A-2-a-iv):

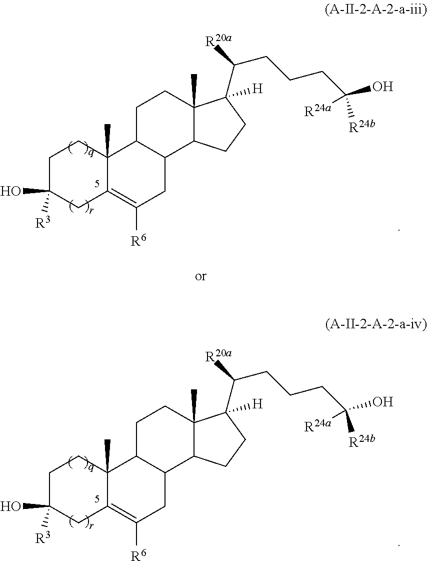

A-226. The compound according to embodiment A-224, wherein the compound of formula (A-II-2-A-2-a-ii) is a compound of formula (A-II-2-A-2-a-v) or formula (A-II-2-A-2-a-vi):

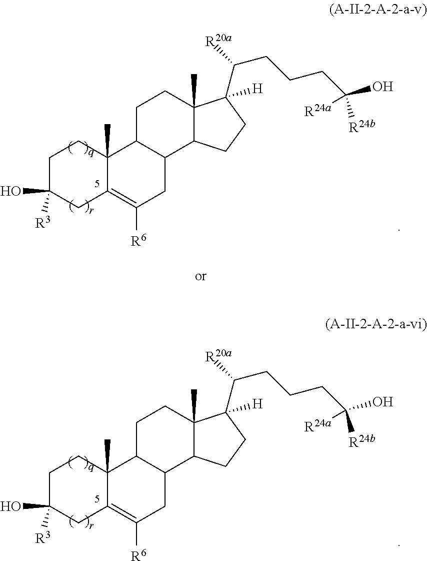

A-227. The compound according to embodiment A-217, wherein the compound of formula (A-II-2-B) is a compound of formula (A-II-2-B-1) or formula (A-II-2-B-2):

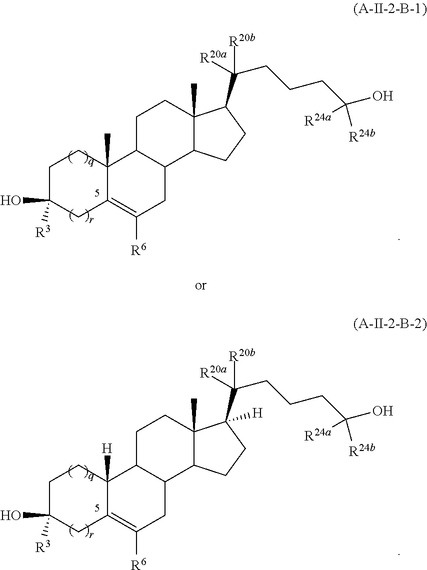

A-228. The compound according to embodiment A-227, wherein the compound of formula (A-II-2-B-1) is a compound of formula (A-II-2-B-1-a):

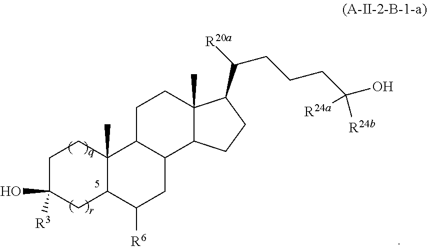

A-229. The compound according to embodiment A-228, wherein the compound of formula (A-II-2-B-1-a) is a compound of formula (A-II-2-B-1-a-i) or formula (A-II-2-B-1-a-ii):

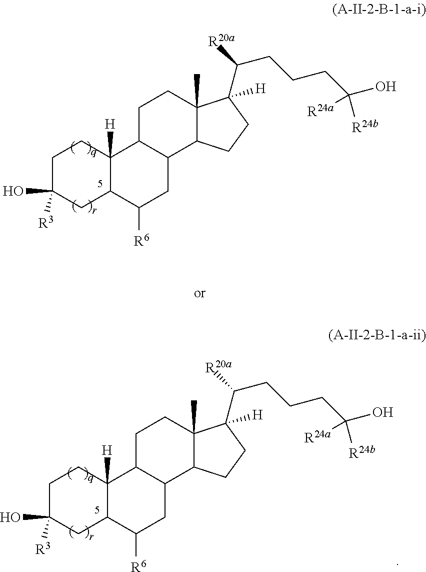

A-230. The compound according to embodiment A-229, wherein the compound of formula (A-II-2-B-1-a-i) is a compound of formula (A-II-2-B-1-a-iii) or formula (A-II-2-B-1-a-iv):

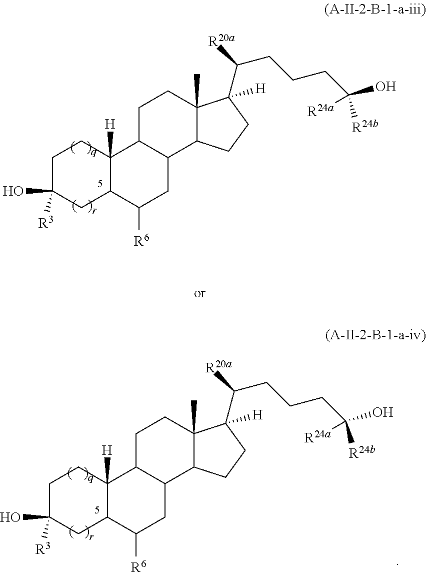

A-231. The compound according to embodiment A-229, wherein the compound of formula (A-II-2-B-1-a-ii) is a compound of formula (A-II-2-B-1-a-v) or formula (A-II-2-B-1-a-vi):

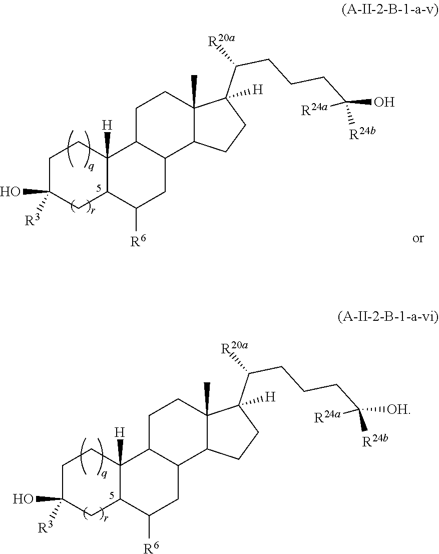

A-232. The compound according to embodiment A-227, wherein the compound of formula (A-II-2-B-2) is a compound of formula (A-II-2-B-2-a):

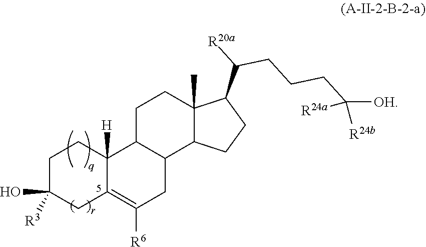

A-233. The compound according to embodiment A-232, wherein the compound of formula (A-II-2-B-2-a) is a compound of formula (A-II-2-B-2-a-i) or formula (A-II-2-B-2-a-ii):

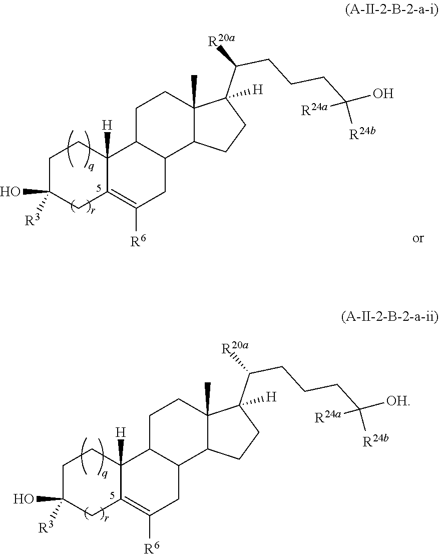

A-234. The compound according to embodiment A-233, wherein the compound of formula (A-II-2-B-2-a-i) is a compound of formula (A-II-2-B-2-a-iii) or formula (A-II-2-B-2-a-iv):

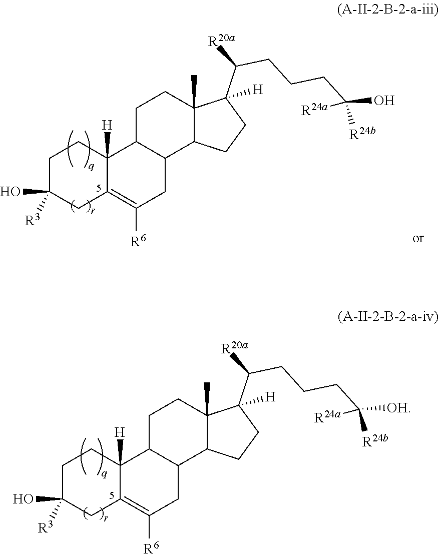

A-235. The compound according to embodiment A-233, wherein the compound of formula (A-II-2-B-2-a-ii) is a compound of formula (A-II-2-B-2-a-v) or formula (A-II-2-B-2-a-vi):

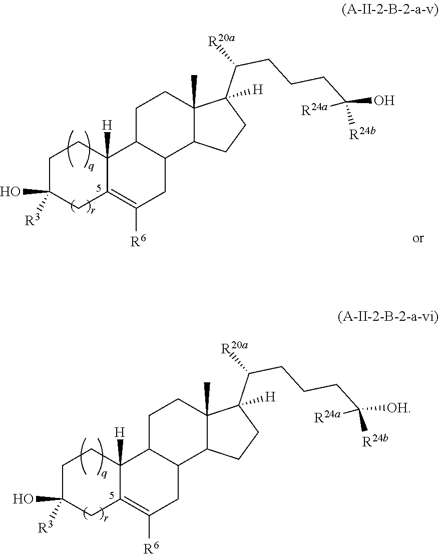

A-236. The compound according to embodiment A-195, wherein the compound of formula (A-I) is a compound of formula (A-III):

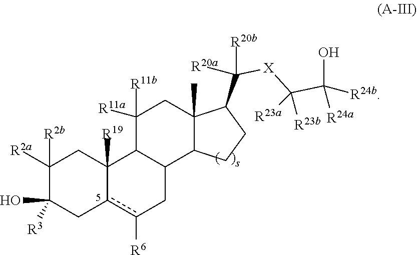

A-237. The compound according to embodiment A-236, wherein the compound of formula (A-III) is a compound of formula (A-III-1) or formula (A-III-2):

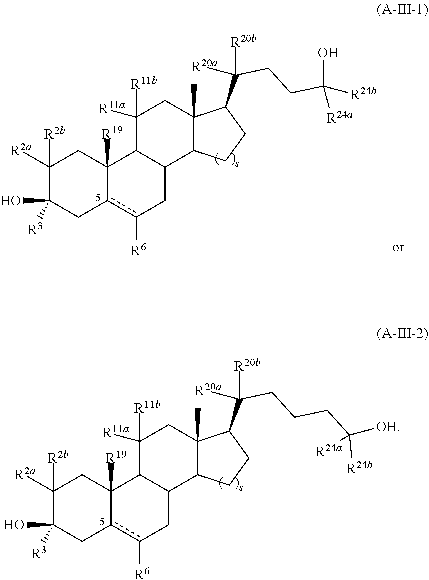

A-238. The compound according to embodiment A-237, wherein the compound of formula (A-III-1) is a compound of formula (A-III-1-A) or formula (A-III-1-B):

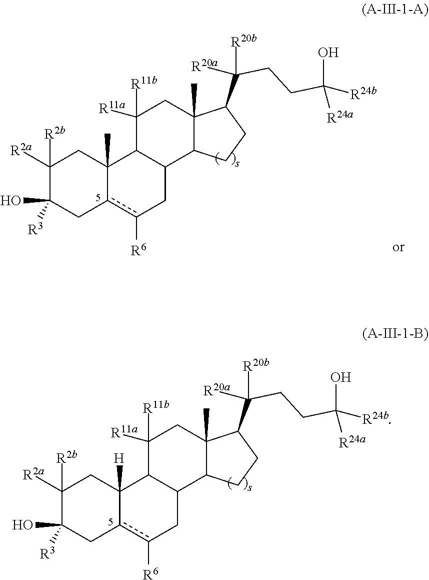

A-239. The compound according to embodiment A-238, wherein the compound of formula (A-III-1-A) is a compound of formula (A-III-1-A-1) or formula (A-III-1-A-2):

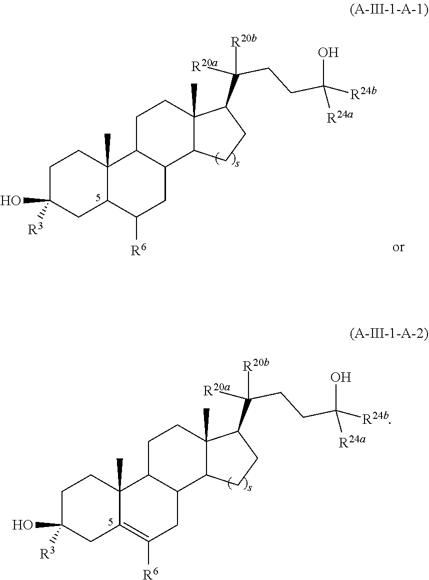

A-240. The compound according to embodiment A-239, wherein the compound of formula (A-III-1-A-1) is a compound of formula (A-III-1-A-1-a):

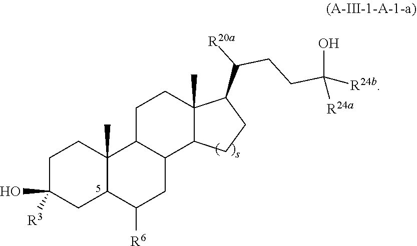

A-241. The compound according to embodiment A-240, wherein the compound of formula (A-III-1-A-1-a) is a compound of formula (A-III-1-A-1-a-i) or (A-III-1-A-1-a-ii):

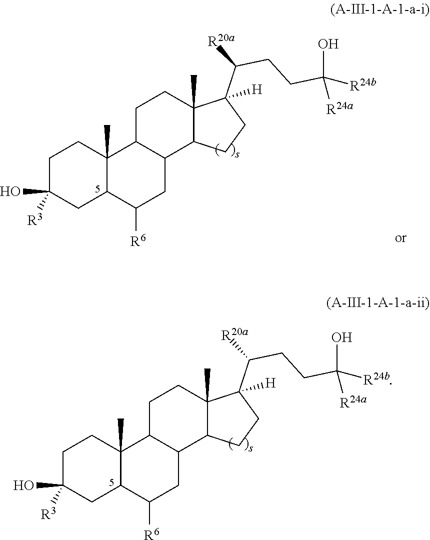

A-242. The compound according to embodiment A-241, wherein the compound of formula (A-III-1-A-1-a-i) is a compound of formula (A-III-1-A-1-a-iii) or (A-III-1-A-1-a-iv):

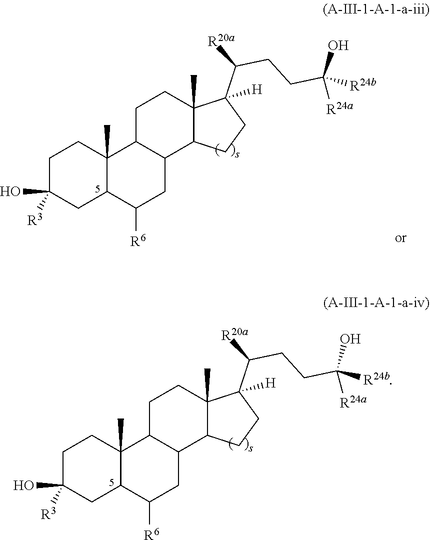

A-243. The compound according to embodiment A-241, wherein the compound of formula (A-III-1-A-1-a-ii) is a compound of formula (A-III-1-A-1-a-v) or (A-III-1-A-1-a-vi):

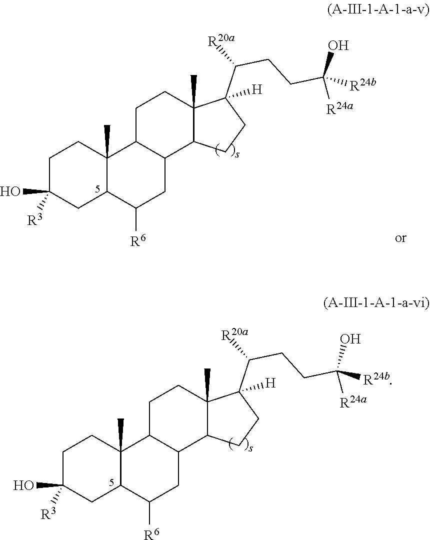

A-244. The compound according to embodiment A-239, wherein the compound of formula (A-III-1-A-2) is a compound of formula (A-III-1-A-2-a):

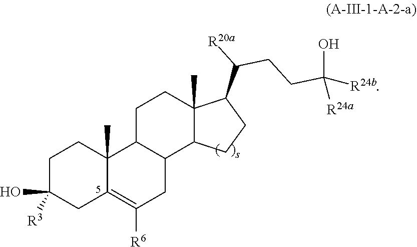

A-245. The compound according to embodiment A-244, wherein the compound of formula (A-III-1-A-2-a) is a compound of formula (A-III-1-A-2-a-i) or (A-III-1-A-2-a-ii):

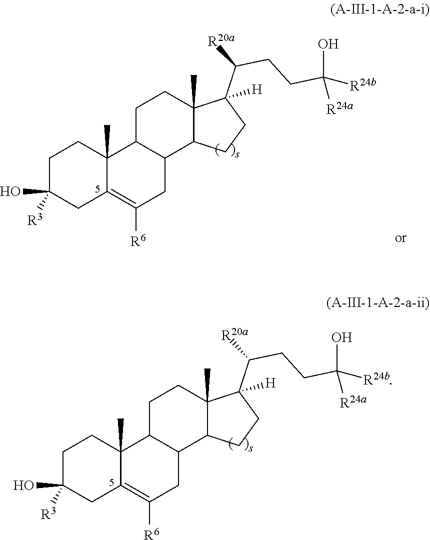

A-246. The compound according to embodiment A-245, wherein the compound of formula (A-III-1-A-2-a-i) is a compound of formula (A-III-1-A-2-a-iii) or (A-III-1-A-2-a-iv):

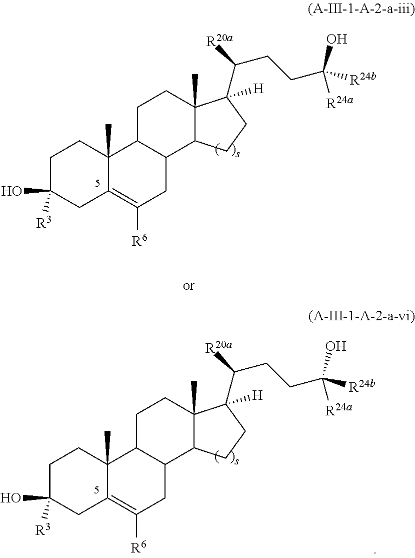

A-247. The compound according to embodiment A-245, wherein the compound of formula (A-III-1-A-2-a-ii) is a compound of formula (A-III-1-A-2-a-v) or (A-III-1-A-2-a-vi):

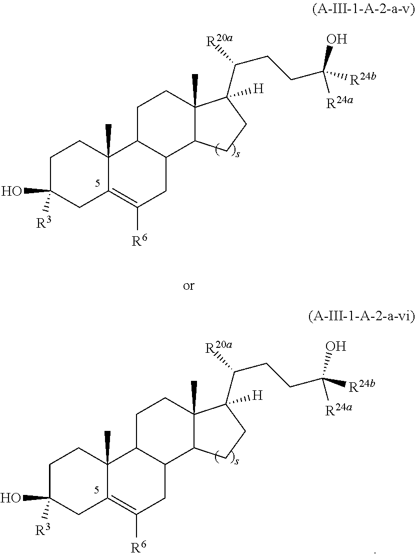

A-248. The compound according to embodiment A-238, wherein the compound of formula (A-III-1-B) is a compound of formula (A-III-1-B-1) or formula (A-III-1-B-2):

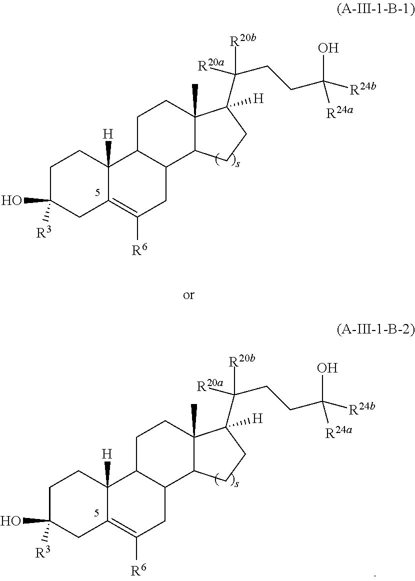

A-249. The compound according to embodiment A-248, wherein the compound of formula (A-III-1-B-1) is a compound of formula (A-III-1-B-1-a):

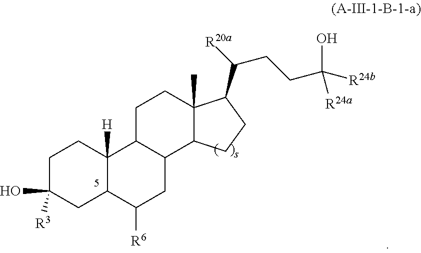

A-250. The compound according to embodiment A-249, wherein the compound of formula (A-III-1-B-1-a) is a compound of formula (A-III-1-B-1-a-i) or formula (A-III-1-B-1-a-ii):

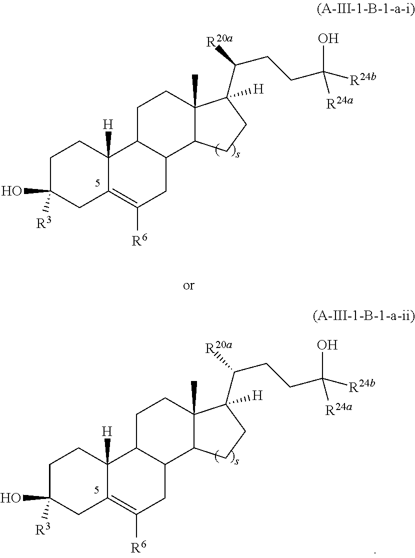

A-251. The compound according to embodiment A-250, wherein the compound of formula (A-III-1-B-1-a-i) is a compound of formula (A-III-1-B-1-a-iii) or formula (A-III-1-B-1-a-iv):

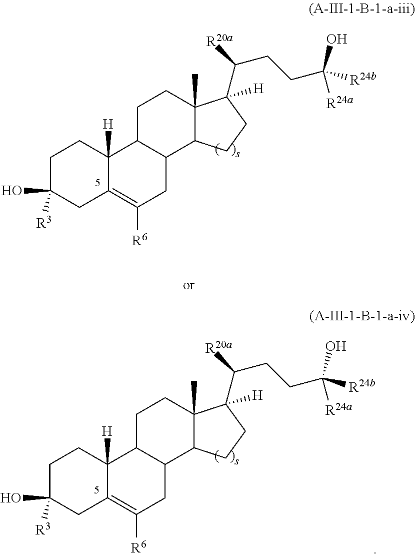

A-252. The compound according to embodiment A-250, wherein the compound of formula (A-III-1-B-1-a-ii) is a compound of formula (A-III-1-B-1-a-v) or formula (A-III-1-B-1-a-vi):

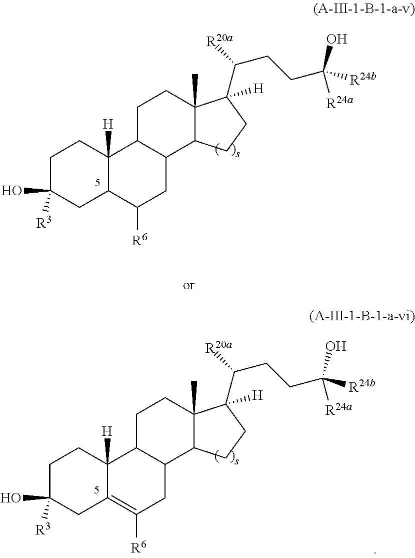

A-253. The compound according to embodiment A-248, wherein the compound of formula (A-III-1-B-2) is a compound of formula (A-III-1-B-2-a):

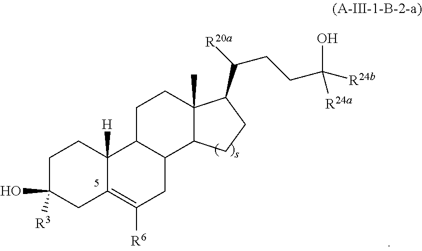

A-254. The compound according to embodiment A-253, wherein the compound of formula (A-III-1-B-2-a) is a compound of formula (A-III-1-B-2-a-i) or formula (A-III-1-B-2-a-ii):

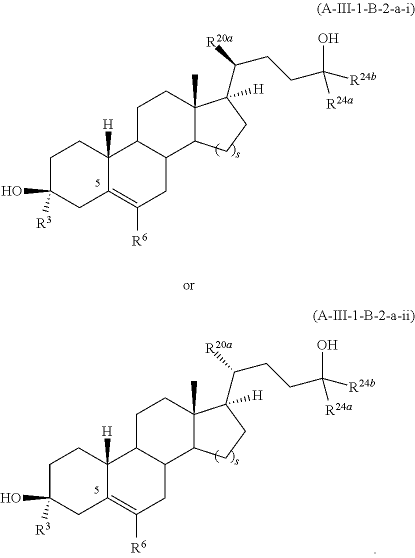

A-255. The compound according to embodiment A-254, wherein the compound of formula (A-III-1-B-2-a-i) is a compound of formula (A-III-1-B-2-a-iii) or formula (A-III-1-B-2-a-iv):

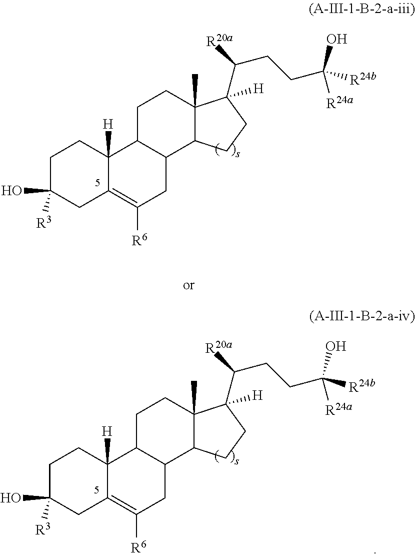

A-256. The compound according to embodiment A-254, wherein the compound of formula (A-III-1-B-2-a-ii) is a compound of formula (A-III-1-B-2-a-v) or formula (A-III-1-B-2-a-vi):

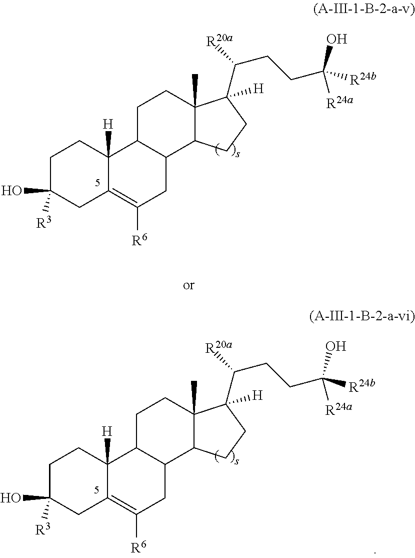

A-257. The compound according to embodiment A-237, wherein the compound of formula (III-2) is a compound of formula (A-III-2-A) or formula (A-III-2-B):

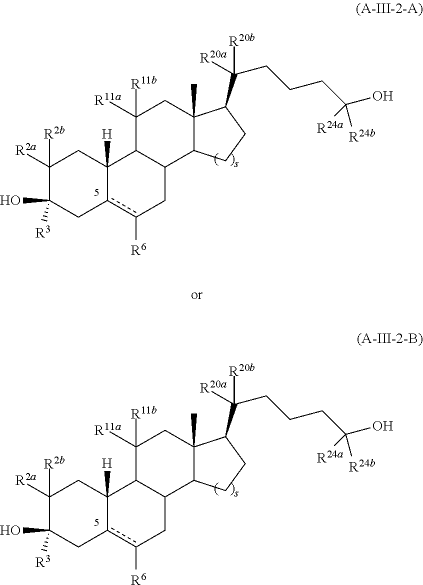

A-258. The compound according to embodiment A-257, wherein the compound of formula (A-III-2-A) is a compound of formula (A-III-2-A-1) or formula (A-III-2-A-2):

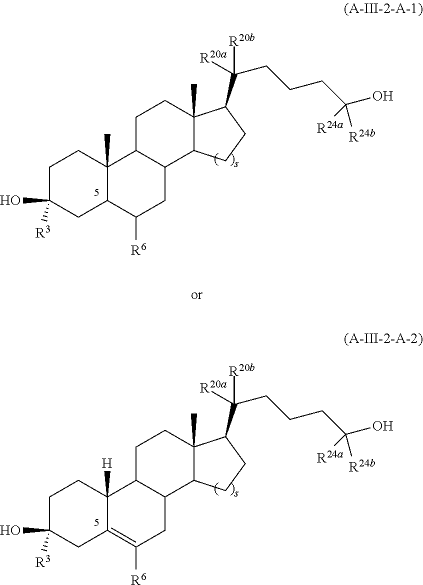

A-259. The compound according to embodiment A-258, wherein the compound of formula (A-III-2-A-1) is a compound of formula (A-III-2-A-1-a):

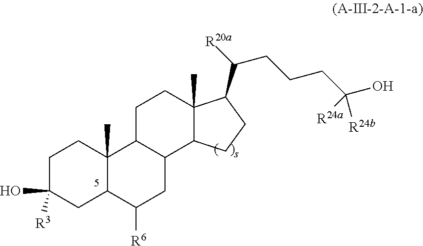

A-260. The compound according to embodiment A-259, wherein the compound of formula (A-III-2-A-1-a) is a compound of formula (A-III-2-A-1-a-i) or formula (A-III-2-A-1-a-ii):

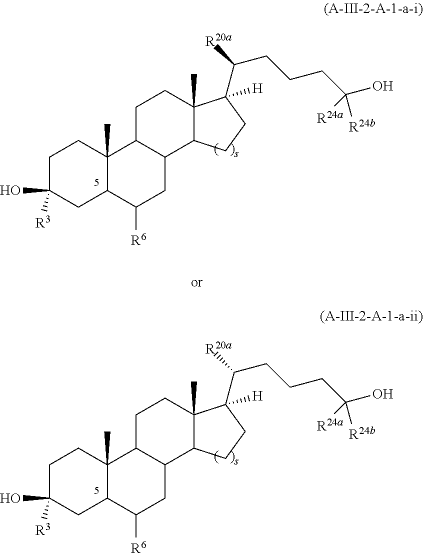

A-261. The compound according to embodiment A-260, wherein the compound of formula (A-III-2-A-1-a-i) is a compound of formula (A-III-2-A-1-a-iii) or formula (A-III-2-A-1-a-iv):

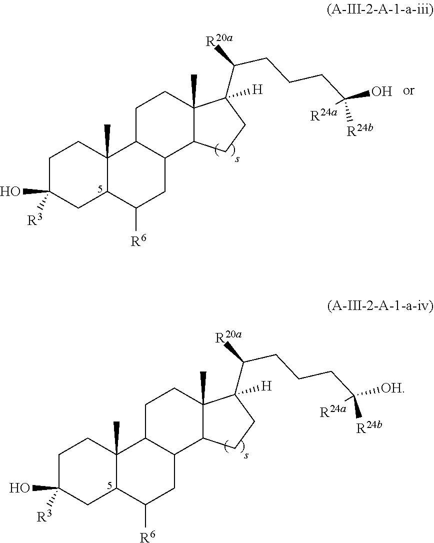

A-262. The compound according to embodiment A-260, wherein the compound of formula (A-III-2-A-1-a-ii) is a compound of formula (A-III-2-A-1-a-v) or formula (A-III-2-A-1-a-vi):

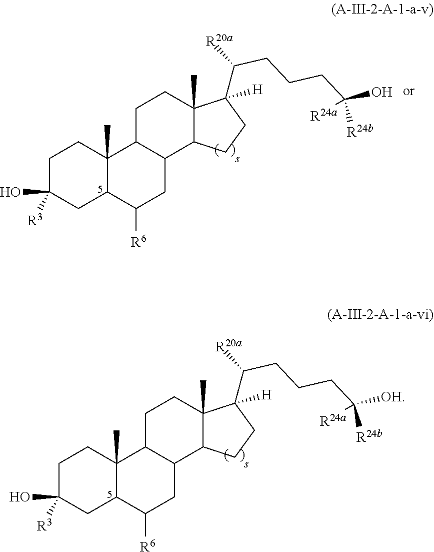

A-263. The compound according to embodiment A-258, wherein the compound of formula (A-III-2-A-2) is a compound of formula (A-III-2-A-2-a):

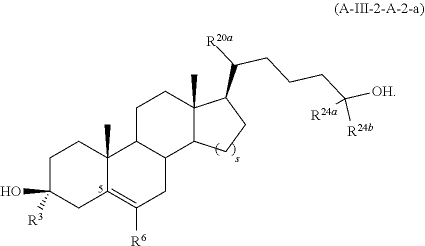

A-264. The compound according to embodiment A-263, wherein the compound of formula (A-III-2-A-2-a) is a compound of formula (A-III-2-A-2-a-i) or formula (A-III-2-A-2-a-ii):

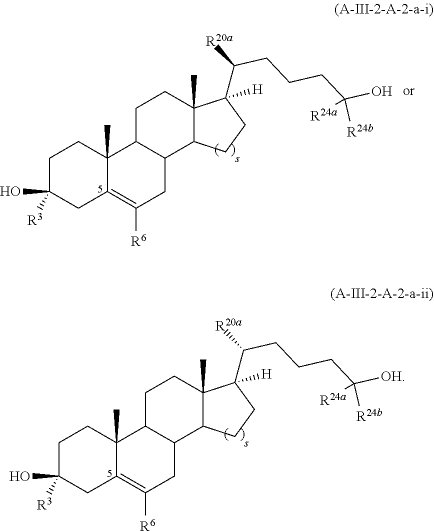

A-265. The compound according to embodiment A-264, wherein the compound of formula (A-III-2-A-2-a-i) is a compound of formula (A-III-2-A-2-a-iii) or formula (A-III-2-A-2-a-iv):

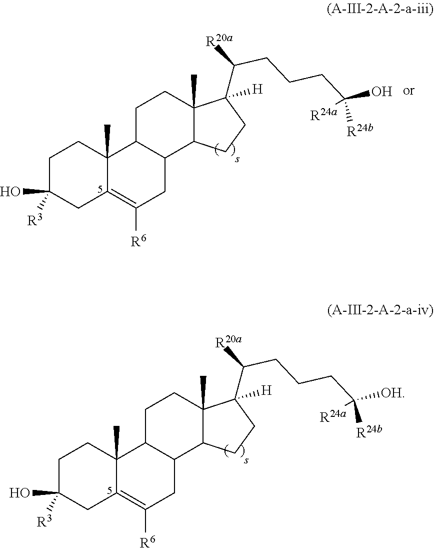

A-266. The compound according to embodiment A-264, wherein the compound of formula (A-III-2-A-2-a-ii) is a compound of formula (A-III-2-A-2-a-v) or formula (A-III-2-A-2-a-vi):

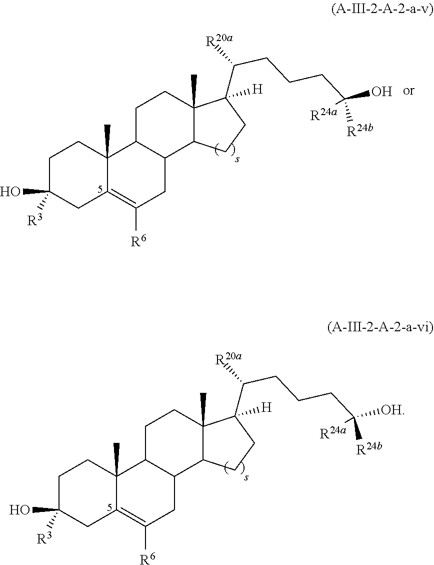

A-267. The compound according to embodiment A-257, wherein the compound of formula (A-III-2-B) is a compound of formula (A-III-2-B-1) or formula (A-III-2-B-2):

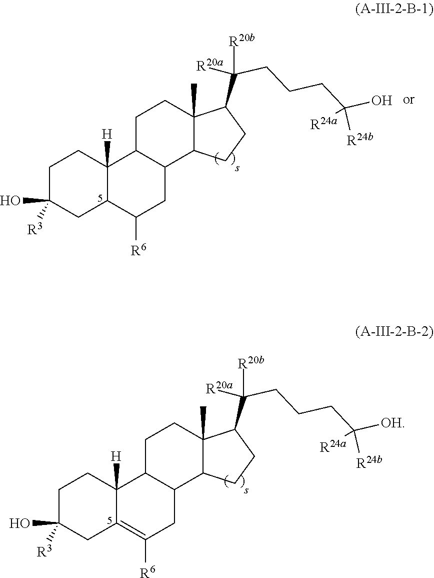

A-268. The compound according to embodiment A-267, wherein the compound of formula (A-III-2-B-1) is a compound of formula (A-III-2-B-1-a):

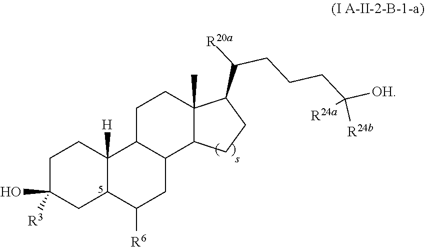

A-269. The compound according to embodiment A-268, wherein the compound of formula (A-III-2-B-1-a) is a compound of formula (A-III-2-B-1-a-i) or formula (A-III-2-B-1-a-ii):

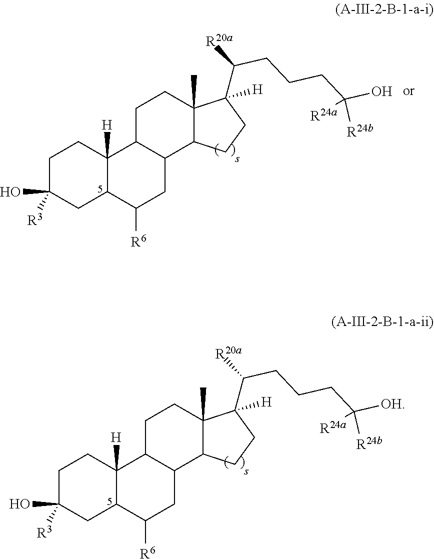

A-270. The compound according to embodiment A-269, wherein the compound of formula (A-III-2-B-1-a-i) is a compound of formula (A-III-2-B-1-a-iii) or formula (A-III-2-B-1-a-iv):

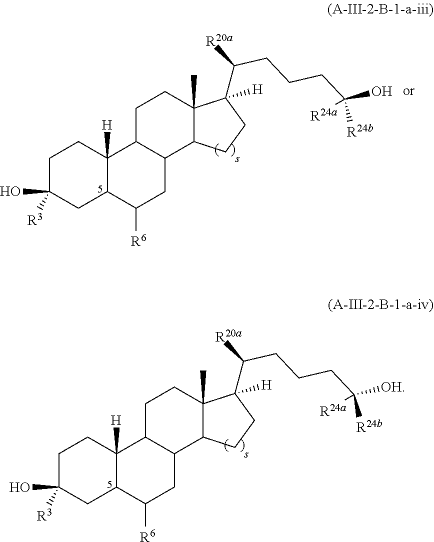

A-271. The compound according to embodiment A-269, wherein the compound of formula (A-III-2-B-1-a-ii) is a compound of formula (A-III-2-B-1-a-v) or formula (A-III-2-B-1-a-vi):

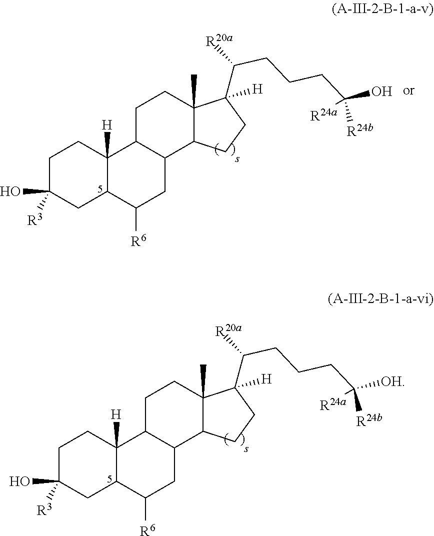

A-272. The compound according to embodiment A-267, wherein the compound of formula (A-III-2-B-2) is a compound of formula (A-III-2-B-2-a):

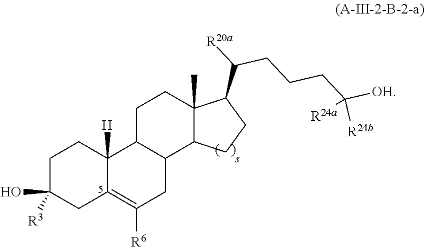

A-273. The compound according to embodiment A-272, wherein the compound of formula (A-III-2-B-2-a) is a compound of formula (A-III-2-B-2-a-i) or formula (A-III-2-B-2-a-ii):

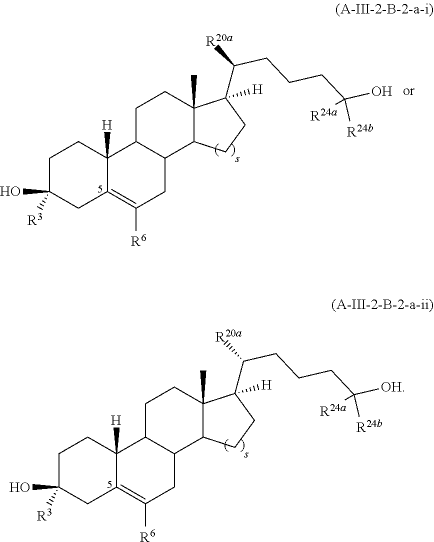

A-274. The compound according to embodiment A-273, wherein the compound of formula (A-III-2-B-2-a-i) is a compound of formula (A-III-2-B-2-a-iii) or formula (A-III-2-B-2-a-iv):

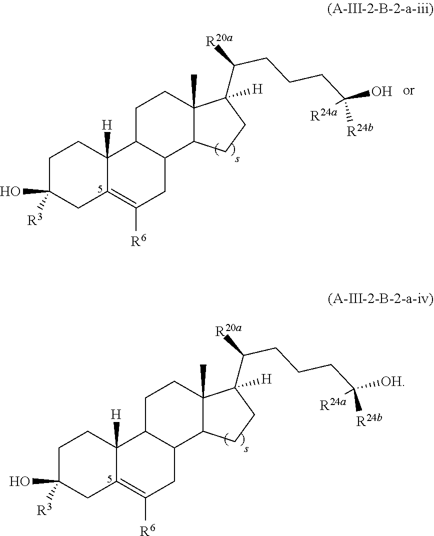

A-275. The compound according to embodiment A-273, wherein the compound of formula (A-III-2-B-2-a-ii) is a compound of formula (A-III-2-B-2-a-v) or formula (A-III-2-B-2-a-vi):

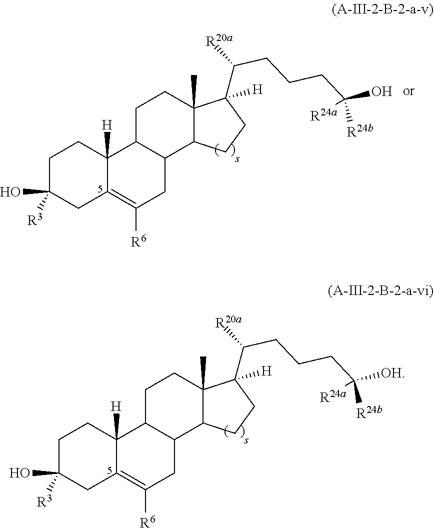

A-276. The compound according to embodiment A-195, wherein the compound of formula (A-I) is a compound of formula (A-IV) or (A-V):

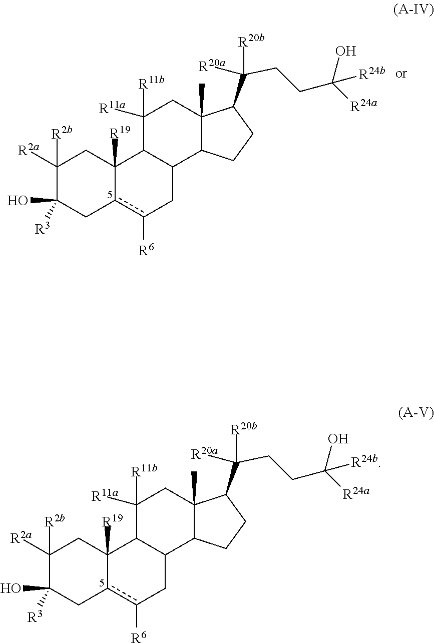

A-277. The compound according to embodiment A-276, wherein the compound of formula (A-IV) is a compound of formula (A-IV-A) or (A-IV-B):

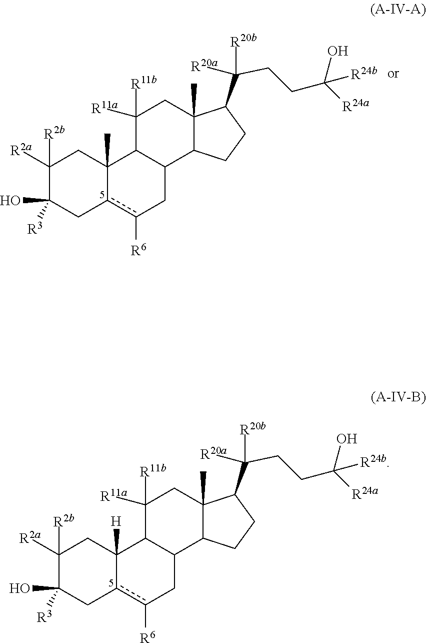

A-278. The compound according to embodiment A-277, wherein the compound of formula (A-IV-A) is a compound of formula (A-IV-A-1) or (A-IV-A-2):

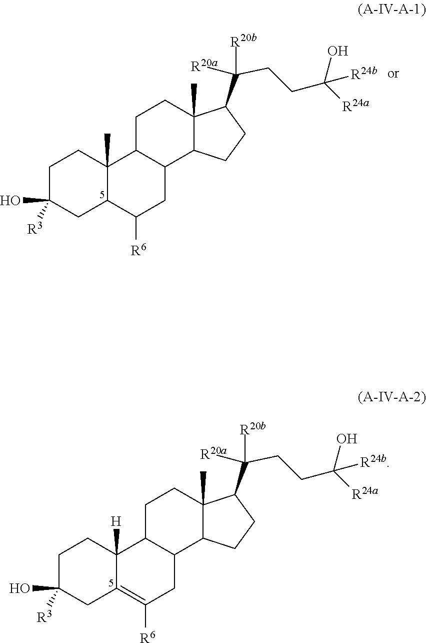

A-279. The compound according to embodiment A-278, wherein the compound of formula (A-IV-A-1) is a compound of formula (A-IV-A-1-a):

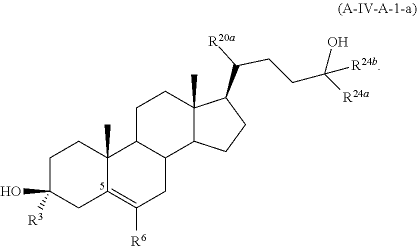

A-280. The compound according to embodiment A-279, wherein the compound of formula (A-IV-A-1-a) is a compound of formula (A-IV-A-1-a-i) or (A-IV-A-1-a-ii):

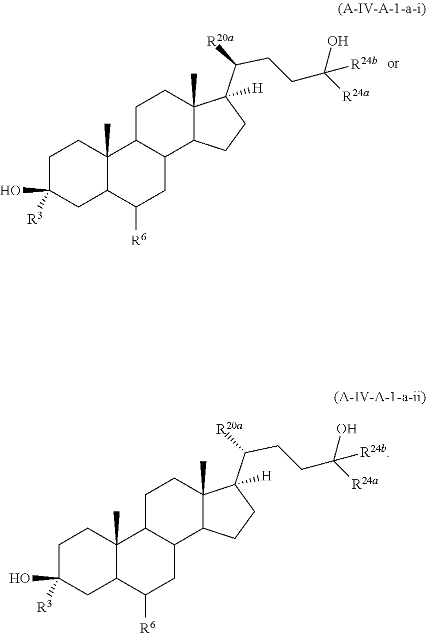

A-281. The compound according to embodiment A-280, wherein the compound of formula (A-IV-A-1-a-i) is a compound of formula (A-IV-A-1-a-iii) or (A-IV-A-1-a-iv):

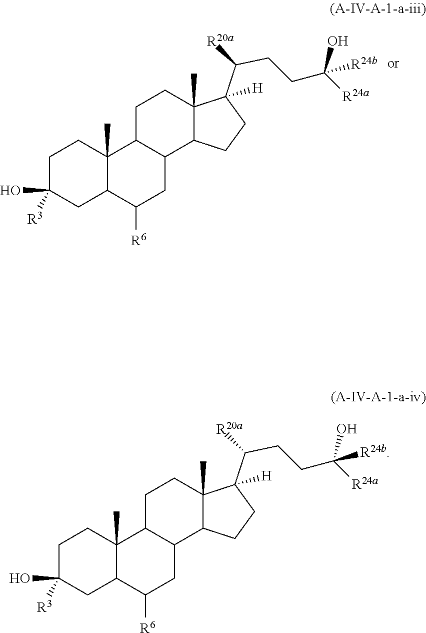

A-282. The compound according to embodiment A-280, wherein the compound of formula (A-IV-A-1-a-ii) is a compound of formula (A-IV-A-1-a-v) or (A-IV-A-1-a-vi):

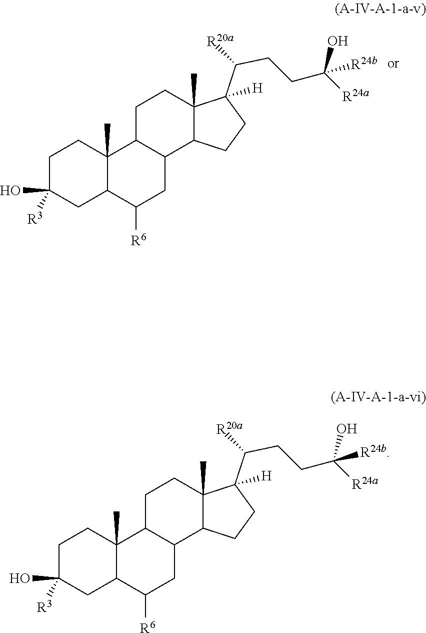

A-283. The compound according to embodiment A-278, wherein the compound of formula (A-IV-A-2) is a compound of formula (A-IV-A-2-a):

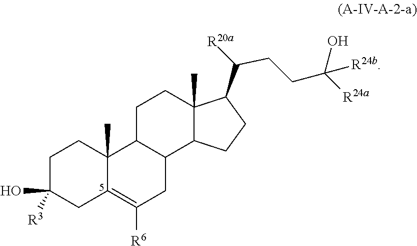

A-284. The compound according to embodiment A-283, wherein the compound of formula (A-IV-A-2-a) is a compound of formula (A-IV-A-2-a-i) or (A-IV-A-2-a-ii):

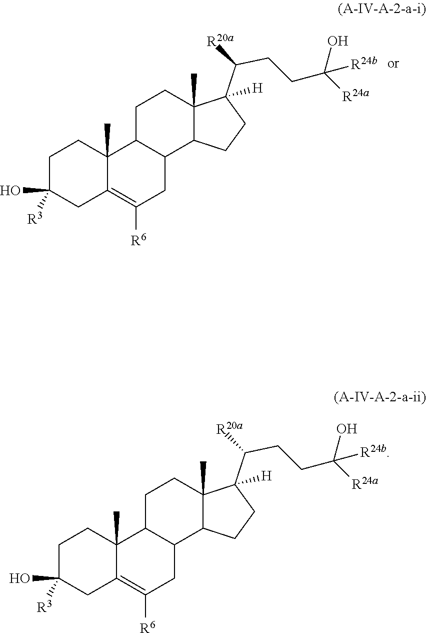

A-285. The compound according to embodiment A-284, wherein the compound of formula (A-IV-A-2-a-i) is a compound of formula (A-IV-A-2-a-iii) or (A-IV-A-2-a-iv):

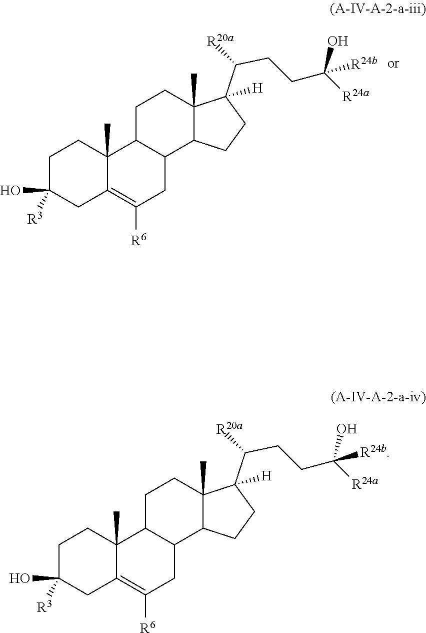

A-286. The compound according to embodiment A-284, wherein the compound of formula (A-IV-A-2-a-ii) is a compound of formula (A-IV-A-2-a-v) or (A-IV-A-2-a-vi):

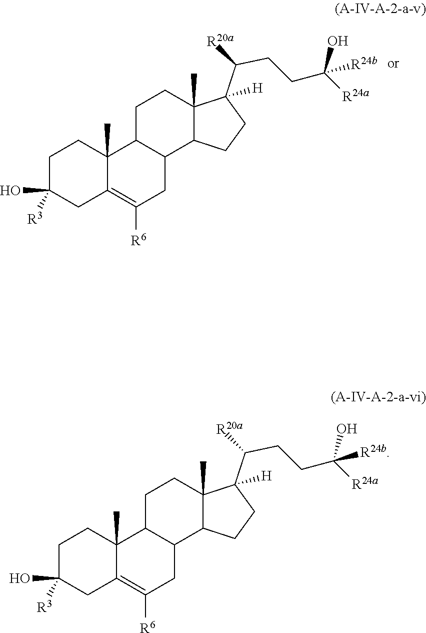

A-287. The compound according to embodiment A-277, wherein the compound of formula (A-IV-B) is a compound of formula (A-IV-B-1) or (A-IV-B-2):

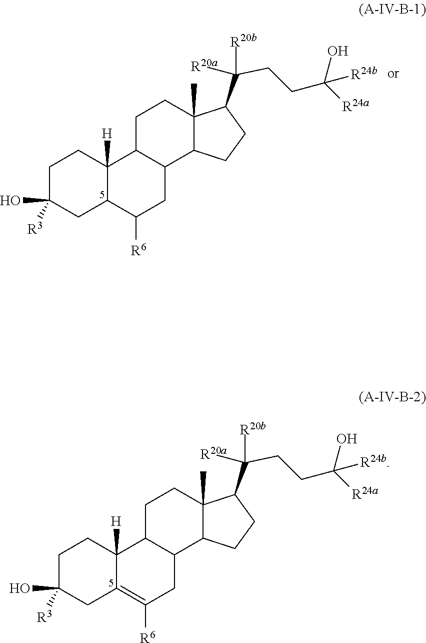

A-288. The compound according to embodiment A-287, wherein the compound of formula (A-IV-B-1) is a compound of formula (A-IV-B-1-a):

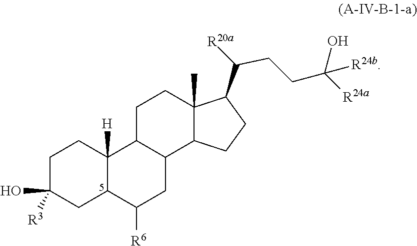

A-289. The compound according to embodiment A-288, wherein the compound of formula (A-IV-B-1-a) is a compound of formula (A-IV-B-1-a-i) or (A-IV-B-1-a-ii):

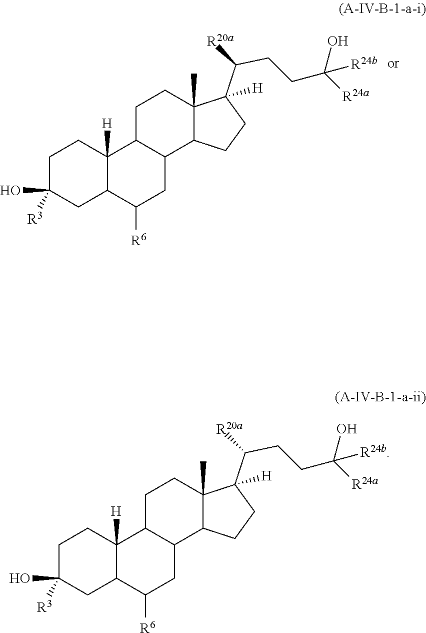

A-290. The compound according to embodiment A-289, wherein the compound of formula (A-IV-B-1-a-i) is a compound of formula (A-IV-B-1-a-iii) or (A-IV-B-1-a-iv):

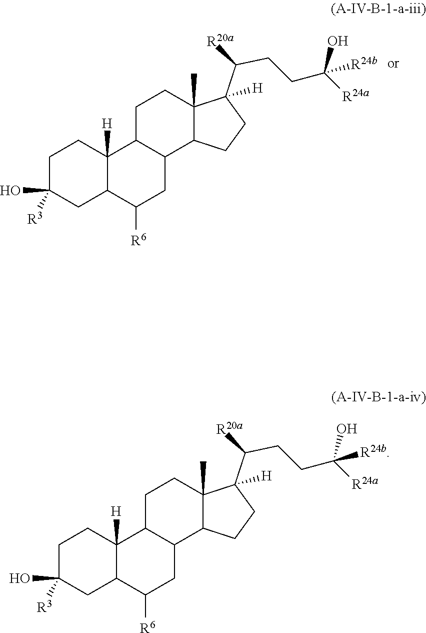

A-291. The compound according to embodiment A-289, wherein, the compound of formula (A-IV-B-1-a-ii) is a compound of formula (A-IV-B-1-a-v) or (A-IV-B-1-a-vi):

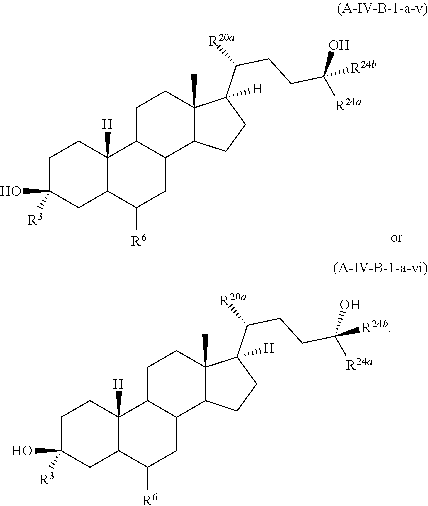

A-292. The compound according to embodiment A-287, wherein the compound of formula (A-IV-B-2) is a compound of formula (A-IV-B-2-a):

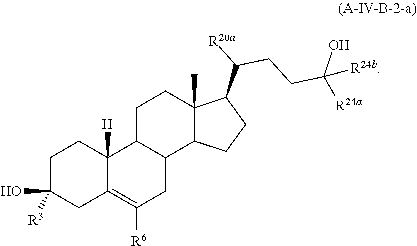

A-293. The compound according to embodiment A-292, wherein the compound of formula (A-IV-B-2-a) is a compound of formula (A-IV-B-2-a-i) or (A-IV-B-2-a-ii):

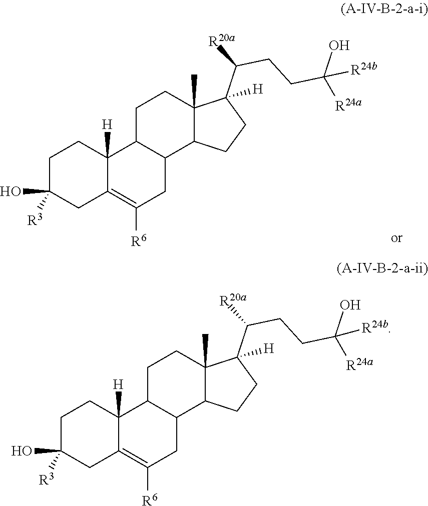

A-294. The compound according to embodiment A-293, wherein the compound of formula (A-IV-B-2-a-i) is a compound of formula (A-IV-B-2-a-iii) or (A-IV-B-2-a-iv):

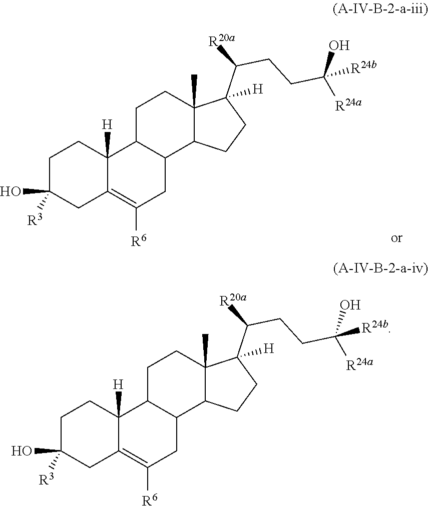

A-295. The compound according to embodiment A-293, wherein the compound of formula (A-IV-B-2-a-ii) is a compound of formula (A-IV-B-2-a-v) or (A-IV-B-2-a-vi):

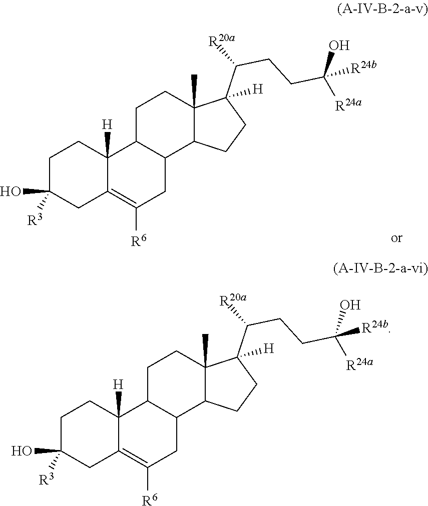

A-296. The compound according to embodiment A-276, wherein the compound of formula (A-V) is a compound of formula (A-V-A) or (A-V-B):

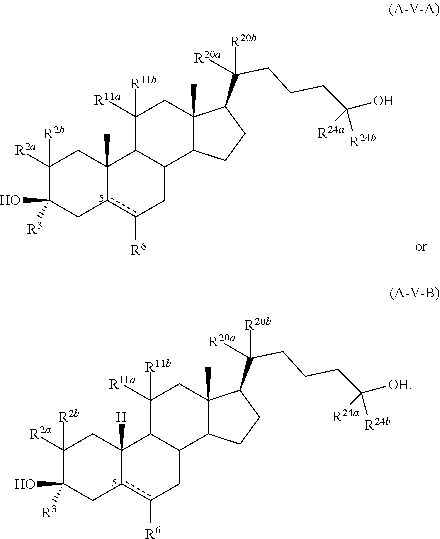

A-297. The compound according to embodiment A-296, wherein the compound of formula (A-V-A) is a compound of formula (A-V-A-1) or (A-V-A-2):

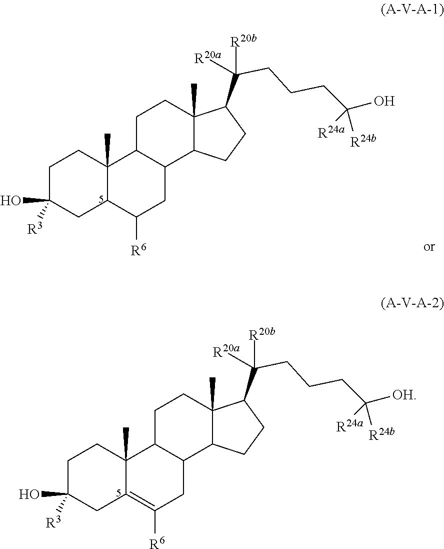

A-298. The compound according to embodiment A-297, wherein the compound of formula (A-V-A-1) is a compound of formula (A-V-A-1-a):

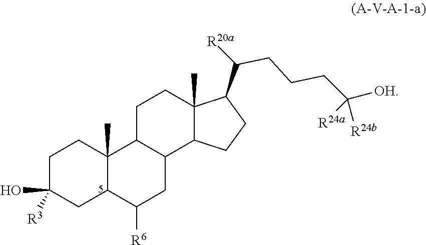

A-299. The compound according to embodiment A-298, wherein the compound of formula (A-V-A-1-a) is a compound of formula (A-V-A-1-a-i) or (A-V-A-1-a-ii):

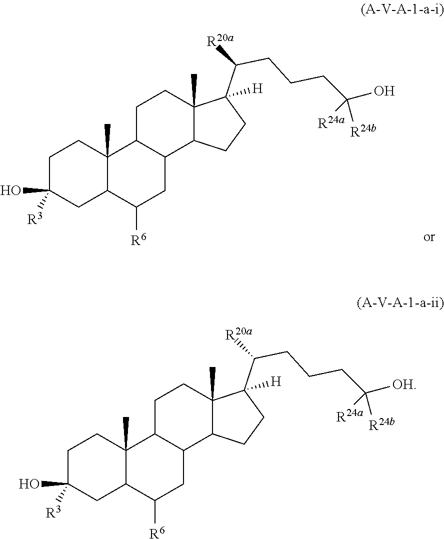

A-300. The compound according to embodiment A-299, wherein the compound of formula (A-V-A-1-a-i) is a compound of formula (A-V-A-1-a-iii) or (A-V-A-1-a-iv):

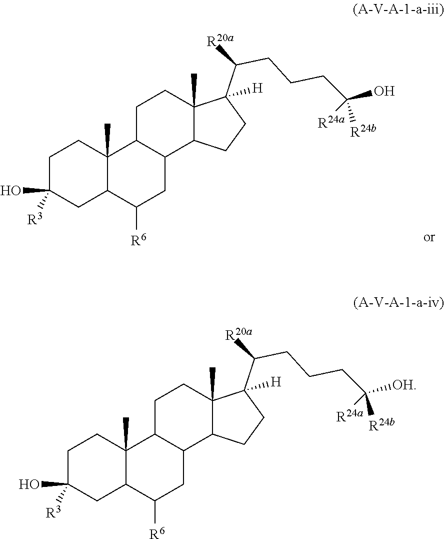

A-301. The compound according to embodiment A-299, wherein the compound of formula (A-V-A-1-a-ii) is a compound of formula (A-V-A-1-a-v) or (A-V-A-1-a-vi):

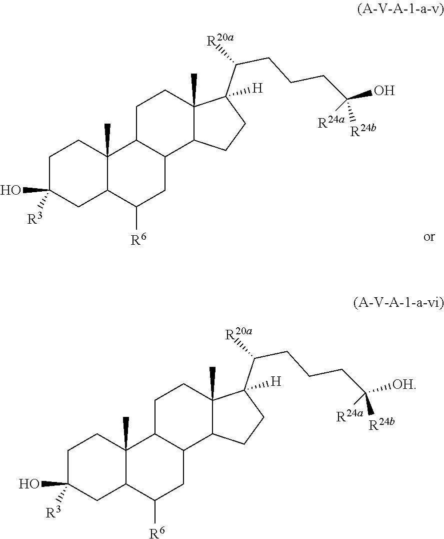

A-302. The compound according to embodiment A-297, wherein the compound of formula (A-V-A-2) is a compound of formula (A-V-A-2-a):

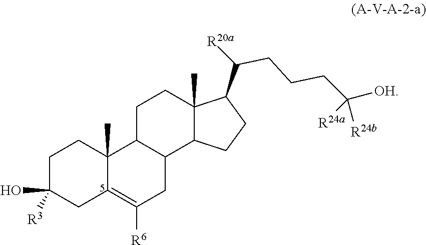

A-303. The compound according to embodiment A-302, wherein the compound of formula (A-V-A-2-a) is a compound of formula (A-V-A-2-a-i) or (A-V-A-2-a-ii):

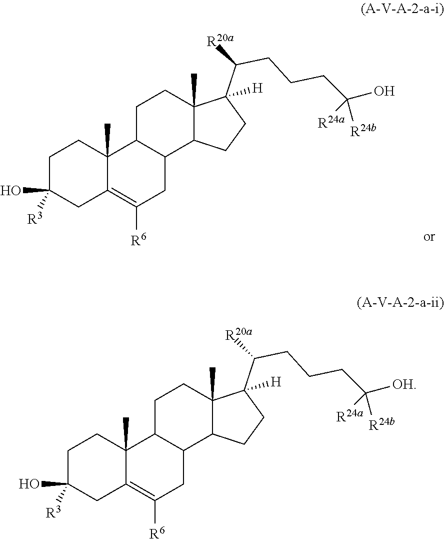

A-304. The compound according to embodiment A-303, wherein the compound of formula (A-V-A-2-a-i) is a compound of formula (A-V-A-2-a-iii) or (A-V-A-2-a-iv):

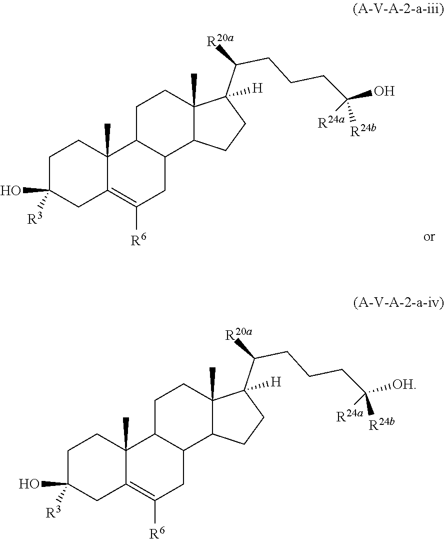

A-305. The compound according to embodiment A-303, wherein the compound of formula (A-V-A-2-a-ii) is a compound of formula (A-V-A-2-a-v) or (A-V-A-2-a-vi):

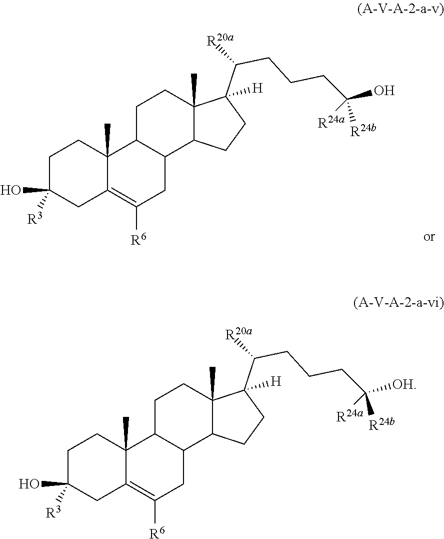

A-306. The compound according to embodiment A-296, wherein the compound of formula (A-V-B) is a compound of formula (A-V-B-1) or (A-V-B-2):

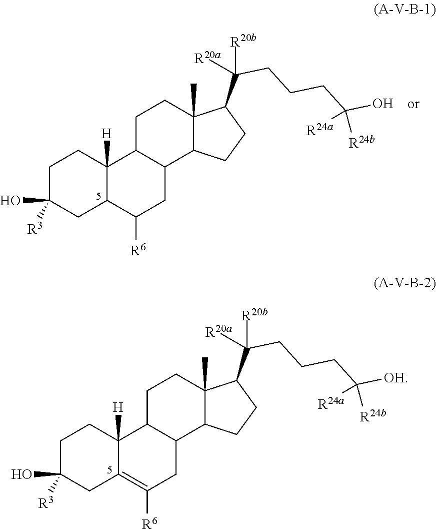

A-307. The compound according to embodiment A-306, wherein the compound of formula (A-V-B-1) is a compound of formula (A-V-B-1-a):

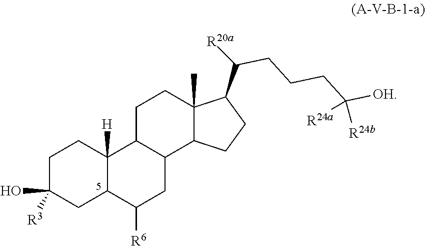

A-308. The compound according to embodiment A-307, wherein the compound of formula (A-V-B-1-a) is a compound of formula (A-V-B-1-a-i) or (A-V-B-1-a-ii):

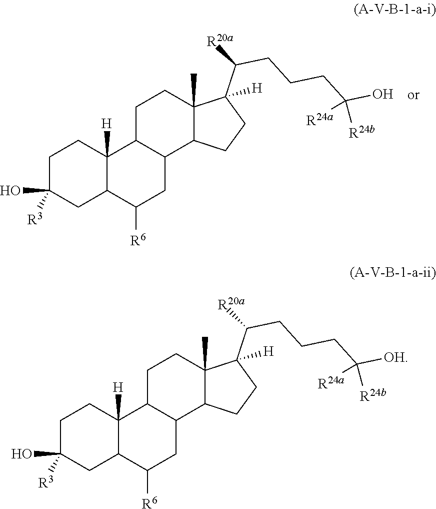

A-309. The compound according to embodiment A-308, wherein the compound of formula (A-V-B-1-a-i) is a compound of formula (A-V-B-1-a-iii) or (A-V-B-1-a-iv):

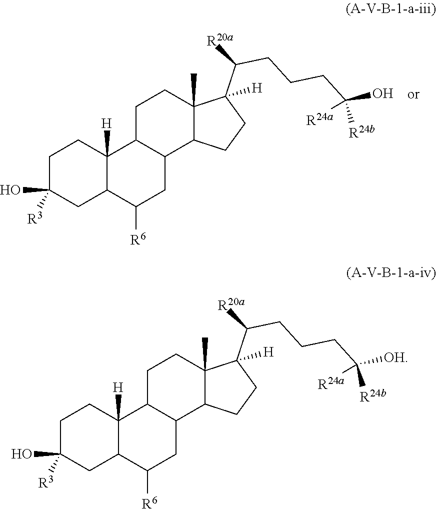

A-310. The compound according to embodiment A-308, wherein the compound of formula (A-V-B-1-a-ii) is a compound of formula (A-V-B-1-a-v) or (A-V-B-1-a-vi):

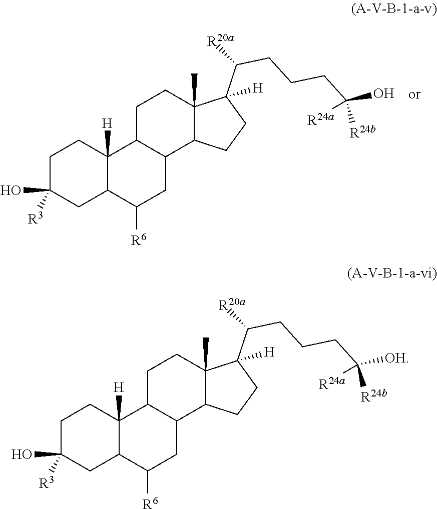

A-311. The compound according to embodiment A-306, wherein the compound of formula (A-V-B-2) is a compound of formula (A-V-B-2-a):

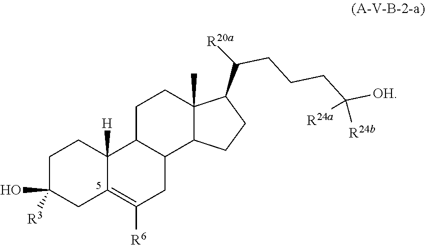

A-312. The compound according to embodiment A-311, wherein the compound of formula (A-V-B-2-a) is a compound of formula (A-V-B-2-a-i) or (A-V-B-2-a-ii):

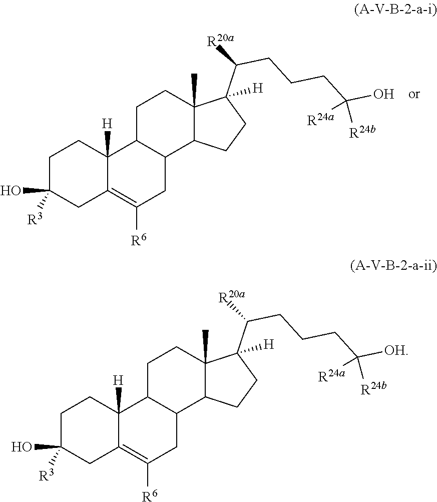

A-313. The compound according to embodiment A-312, wherein the compound of formula (A-V-B-2-a-i) is a compound of formula (A-V-B-2-a-iii) or (A-V-B-2-a-iv):

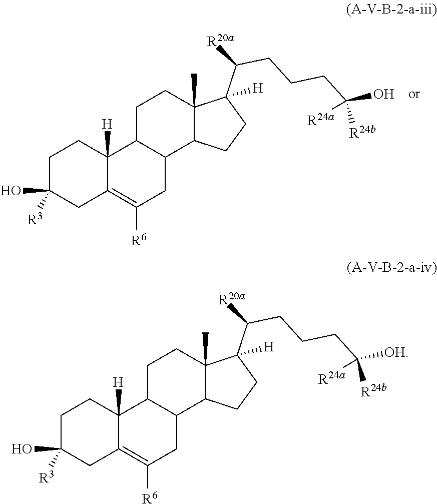

A-314. The compound according to embodiment A-312, wherein the compound of formula (A-V-B-2-a-ii) is a compound of formula (A-V-B-2-a-v) or (A-V-B-2-a-vi):

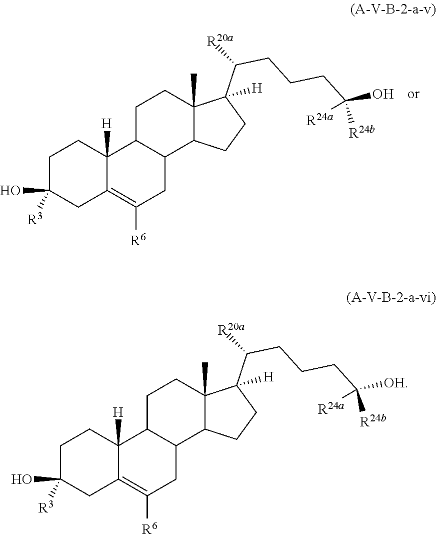

A-315. The compound according to embodiment A-195, wherein the compound of formula (A-I) is a compound of formula (A-VI):

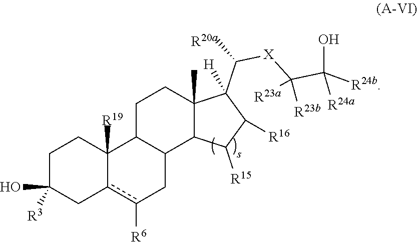

A-316. The compound according to embodiment A-195, wherein the compound of formula (A-I) is a compound of formula (A-VII):

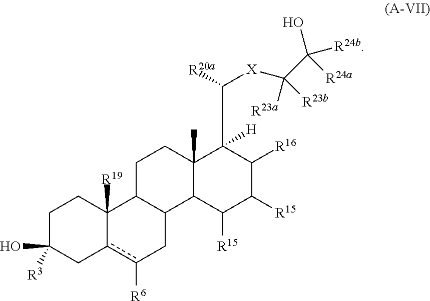

A-317. The compound according to embodiment A-195, wherein the compound of formula (A-I) is a compound of formula (A-VIII):

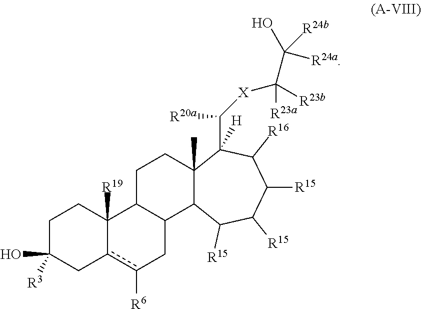

A-318. The compound according to embodiment A-195, wherein the compound of formula (A-I) is a compound of formula (A-IX):

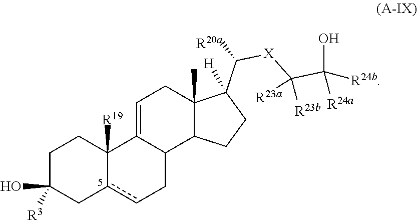

A-319. The compound according to embodiment A-195, wherein the compound of formula (A-I) is a compound of formula (A-X):

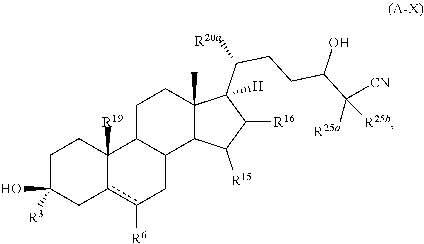

wherein:
R^{25a} is selected from the group consisting of substituted or unsubstituted C₁₋₆-alkyl, substituted or unsubstituted C₂₋₆alkenyl, substituted or unsubstituted C₂₋₆ alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl;
R^{25b} is hydrogen, substituted or unsubstituted C₁₋₆alkyl group, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, or substituted or unsubstituted 5- to 10-membered heteroaryl; or
R^{25a} and R^{25b}, taken together with the carbon atom to which they are attached, form a substituted or unsubstituted C₃₋₆carbocyclyl; and
R³, R⁶, R¹⁵, R¹⁶, R¹⁹, and R^{20a} are as defined in embodiment A-194.

A-320. The compound according to embodiment A-195, wherein the compound of formula (I) is a compound of formula (A-XI):

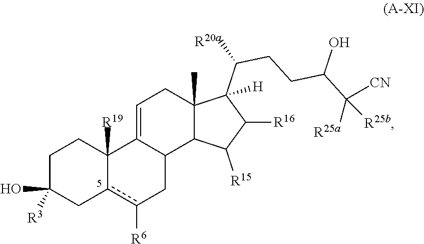

wherein:
R^{25a} is selected from the group consisting of substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl;
R^{25b} is hydrogen, substituted or unsubstituted C₁₋₆alkyl group, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, or substituted or unsubstituted 5- to 10-membered heteroaryl; or
R^{25a} and R^{25b}, taken together with the carbon atom to which they are attached, form a substituted or unsubstituted C₃₋₆carbocyclyl; and
R³, R⁶, R¹⁵, R¹⁶, R¹⁹, and R^{20a} are as defined in embodiment A-194.

A-321. The compound according to embodiment A-195, wherein the compound of formula (A-I) is a compound of formula (A-XII):

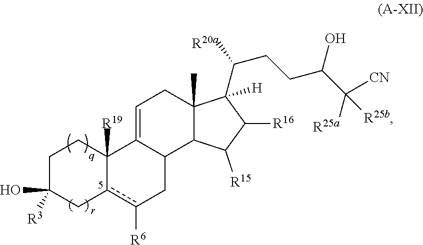

wherein:
R^{25a} is selected from the group consisting of substituted or unsubstituted C₁₋₆-alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl;
R^{25b} is hydrogen, substituted or unsubstituted C₁₋₆alkyl group, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, or substituted or unsubstituted 5- to 10-membered heteroaryl; or
R^{25a} and R^{25b}, taken together with the carbon atom to which they are attached, form a substituted or unsubstituted C₃₋₆carbocyclyl; and
R³, R⁶, R¹⁵, R¹⁶, R¹⁹, and R^{20a} are as defined in embodiment A-194.

A-322. The compound according to embodiment A-321, wherein the compound of formula (A-XII) is a compound of formula (A-XII-A) or (A-XII-B):

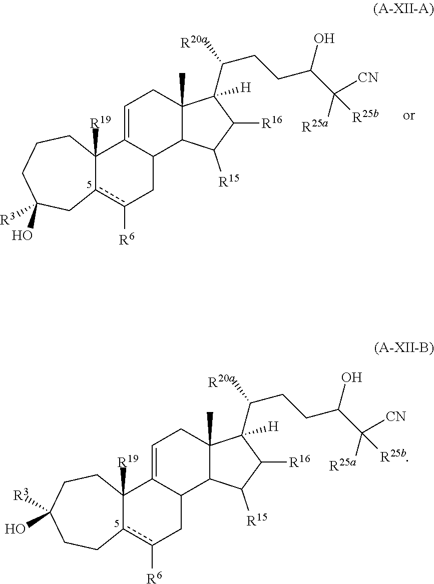

A-323. The compound according to embodiment A-195, wherein the compound of formula (A-I) is a compound of formula (A-XIII):

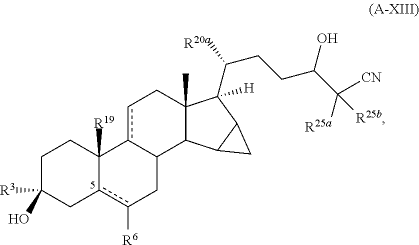

wherein:
R^{25a} is selected from the group consisting of substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl;
R^{25b} is hydrogen, substituted or unsubstituted C₁₋₆alkyl group, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, or substituted or unsubstituted 5- to 10-membered heteroaryl; or
R^{25a} and R^{25b}, taken together with the carbon atom to which they are attached, form a substituted or unsubstituted C₃₋₆carbocyclyl; and
R³, R⁶, R¹⁹, and R^{20a} are as defined in embodiment A-194.

A-324. The compound according to any one of embodiments A-195 to A-198, A-217, A-236 to A-238, A-257, A-276 to A-277, and A-296, wherein each of R^{2a} and R^{2b} is independently hydrogen.

A-325. The compound according to any one of embodiments A-195 to A-198, A-217, A-236 to A-238, A-257, A-276 to A-277, and A-296, wherein each of R^{11a} and R^{11b} is independently hydrogen.

A-326. The compound according to any one of embodiments A-195 to A-323, wherein R³ is unsubstituted C₁₋₆alkyl.

A-327. The compound according to embodiment A-326, wherein R³ is —CH₃, —CH₂CH₃, or —CH₂CH₂CH₃.

A-328. The compound according to embodiment A-326, wherein R³ is —CH₂CH₃.

A-329. The compound according to any one of embodiments A-195 to A-323, wherein R⁶ is hydrogen.

A-330. The compound according to any one of embodiments A-195 to A-323, wherein R^{20a} is halogen, cyano, substituted C₁₋₆alkyl, unsubstituted C₂₋₆alkyl, or substituted or unsubstituted C₁₋₆alkoxy.

A-331. The compound according to embodiment A-330, wherein R^{20a} is halogen.

A-332. The compound according to embodiment A-331, wherein R^{20a} is —F.

A-333. The compound according to embodiment A-330, wherein R^{20a} is substituted C₁₋₆alkyl.

A-334. The compound according to embodiment A-333, wherein R^{20a} is —CF₃.

A-335. The compound according to embodiment A-330, wherein R^{20a} is unsubstituted C₂₋₆alkyl.

A-336. The compound according to embodiment A-335, wherein R^{20a} is —CH₂CH₃.

A-337. The compound according to embodiment A-330, wherein R^{20a} is unsubstituted C₁₋₆alkoxy.

A-338. The compound according to embodiment A-337, wherein R^{20a} is —OCH₃ or —OCH₂CH₃.

A-339. The compound according to embodiment A-330, wherein R^{20a} is cyano.

A-340. The compound according to any one of embodiments A-195 to A-199, A-208, A-217 to A-218, A-227, A-236 to A-239, A-248, A-257 to A-258, A-267, A-276 to A-278, A-287, A-296 to A-297, and A-306, wherein R^{20b} is hydrogen, halogen, or substituted or unsubstituted alkyl C₁₋₆alkyl.

A-341. The compound according to any one of embodiments A-195 to A-196 and A-315 to A-318, wherein X is —(C(R^{X})₂—, wherein R^{X} is hydrogen or fluorine.

A-342. The compound according to embodiment A-341, wherein X is —CH₂—.

A-343. The compound according to embodiment A-341, wherein X is —CH₂CH₂—.

A-344. The compound according to any one of embodiments A-195 to A-196 and A-315 to A-318, wherein each of R^{23a} and R^{23b} is hydrogen.

A-345. The compound according to any one of embodiments A-195 to A-196 and A-315 to A-318, wherein each of R^{23a} and R^{23b} is fluorine.

A-346. The compound according to any one of embodiments A-195 to A-318, wherein R^{24a} is substituted or unsubstituted C₁₋₆alkyl.

A-347. The compound according to embodiment A-346, wherein R^{24a} is —CH₃, —CH(CH₃)₂, or —CF₃.

A-348. The compound according to any one of embodiments A-195 to A-318, wherein R^{24b} is hydrogen or unsubstituted C₁₋₆alkyl.

A-349. The compound according to embodiment A-348, wherein R^{24b} is hydrogen.

A-350. The compound according to embodiment A-348, wherein R^{24b} is —CH₃.

A-351. The compound according to embodiment A-195, wherein the bond between C5 and C6 is a single bond and R⁵ is hydrogen in the alpha configuration.

A-352. The compound according to embodiment A-195, wherein the bond between C9 and C11 is a double bond, R⁹ is absent and R^{11a} is selected from the group consisting of hydrogen, halogen, substituted or unsubstituted C₁₋₆alkyl, and —OR^{A11}, wherein R^{A11} is hydrogen or substituted or unsubstituted C₁₋₆alkyl, and R^{11b} is absent or hydrogen.

A-353. The compound according to embodiment A-195, wherein the bond between C9 and C11 is a single bond, R⁹ is hydrogen and R¹¹ is selected from the group consisting of halogen or substituted or unsubstituted C₁₋₆alkyl.

A-354. The compound according to any one of embodiments A-195, A-315 to A-317, and A-319 to A-322 wherein each instance of R¹⁵ is independently hydrogen, substituted or unsubstituted C₁₋₆alkyl or substituted or unsubstituted C₃₋₆carbocyclyl,

A-355. The compound according to embodiment A-354, wherein each instance of R¹⁵ is independently hydrogen, unsubstituted C₁₋₆alkyl or unsubstituted C₃₋₆carbocyclyl.

A-356. The compound according to embodiment A-355, wherein each instance of R¹⁵ is independently hydrogen, —CH₃ or cyclopropyl.

A-357. The compound according to embodiment A-356, wherein each instance of R¹⁵ is independently hydrogen.

A-358. The compound according to embodiment A-356, wherein each instance of R¹⁵ is independently —CH₃.

A-359. The compound according to embodiment A-356, wherein each instance of R¹⁵ is independently cyclopropyl.

A-360. The compound according to any one of embodiments A-195, A-315 to A-317, and A-319 to A-322, wherein R¹⁶ is hydrogen, substituted or unsubstituted C₁₋₆alkyl or substituted or unsubstituted C₃₋₆carbocyclyl.

A-361. The compound according to embodiment A-360, wherein R 16 is hydrogen or unsubstituted C₁₋₆alkyl.

A-362. The compound according to embodiment A-361, wherein R¹⁶ is hydrogen.

A-363. The compound according to embodiment A-361, wherein R¹⁶ is —CH₃.

A-364. The compound according to any one of embodiments A-195, A-315 to A-317, and A-319 to A-322, wherein R¹⁵ and R¹⁶ are taken together to form a substituted or unsubstituted C₃₋₆carbocyclyl.

A-365. The compound according to embodiment A-364, wherein R¹⁵ and R¹⁶ are taken together to form a cyclopropyl.

A-366. The compound according to embodiment A-195, wherein R¹⁸ is hydrogen, unsubstituted C₁₋₆alkyl or C₁₋₆alkyl substituted with OR^{A18}, wherein R^{A18} is substituted or unsubstituted C₁₋₆alkyl.

A-367. The compound according to embodiment A-366, wherein R 18 is hydrogen, —CH₃, CH₂OCH₃, —OCH₃, —CH₂CH₃, or —CH(CH₃)₂.

A-368. The compound according to embodiment A-367, wherein R¹⁸ is hydrogen or —CH₃.

A-369. The compound according to embodiment A-368, wherein R¹⁸ is —CH₃.

A-370. The compound according to embodiment A-368, wherein R¹⁸ is hydrogen.

A-371. The compound according to any one of embodiments A-195 to A-197, A-236 to A-237, A-276, and A-315 to A-323, wherein R¹⁹ is hydrogen, unsubstituted C₁₋₆ alkyl or C₁₋₆alkyl substituted with OR^{A19}, wherein R^{A19} is substituted or unsubstituted C₁₋₆ alkyl.

A-372. The compound according to embodiment A-371, wherein R¹⁹ is hydrogen, —CH₃, CH₂OCH₃, —OCH₃, —CH₂CH₃, or —CH(CH₃)₂.

A-373. The compound according to embodiment A-372, wherein R¹⁹ is hydrogen or —CH₃.

A-374. The compound according to embodiment A-373, wherein R¹⁹ is —CH₃.

A-375. The compound according to embodiment A-373, wherein R¹⁹ is hydrogen.

A-376. The compound according to embodiment A-195, wherein the compound of formula (A-I) is selected from the group consisting of compounds A-1-A-26 of Table A-1.

A-377. The compound according to embodiment A-195, wherein the compound of formula (A-I) is selected from the group consisting of compounds A-1-A-22 of Table A-1.

A-378. The compound according to embodiment A-377, wherein the compound of formula (A-I) is selected from the group consisting of compounds A-3, A-4, A-5, A-6, A-7, A-9, A-13, A-14, A-15, A-16 of Table A-1.

A-379. A pharmaceutical composition comprising a compound according to any one of embodiments A-195 to A-378, and a pharmaceutically acceptable carrier.

A-380. A method for treating a CNS-related condition in a subject, comprising administering to the subject an effective amount of a compound according to any one of embodiments A-195 to A-378, or a pharmaceutical composition according to embodiment A-379.

A-381. The method according to embodiment A-380, wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease) and tinnitus.

A-382. A method of inducing sedation or anesthesia in a subject, comprising administering to the subject an effective amount of a compound according to any one of embodiments A-195 to A-378, or a pharmaceutical composition according to embodiment A-379.

A-383. A compound according to any one of embodiments A-195 to A-378 or a pharmaceutical composition according to embodiment A-379 for use in treating a CNS-related condition in a subject.

A-384. The compound or pharmaceutical composition for use according to embodiment A-383, wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease) and tinnitus.

A-385. A compound according to any one of embodiments A-195 to A-378 or a pharmaceutical composition according to embodiment A-379 for use in inducing sedation or anesthesia in a subject.

A-386. Use of a compound according to any one of embodiments A-195 to A-378, or a pharmaceutical composition according to embodiment A-379 for the manufacture of a medicament for treating a CNS-related condition in a subject.

A-387. The use according to embodiment A-386, wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease) and tinnitus.

A-388. Use of a compound according to any one of embodiments A-195 to A-378 or a pharmaceutical composition according to embodiment A-379 for the manufacture of a medicament for inducing sedation or anesthesia in a subject.

B-1. A compound of Formula (B-I):

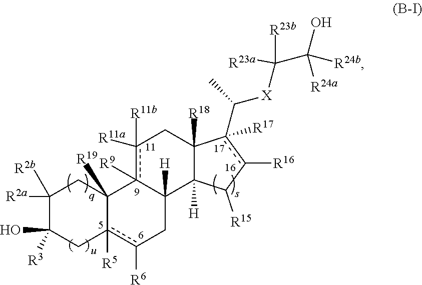

or a pharmaceutically acceptable salt thereof;
wherein:
   each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein R^{A2} is hydrogen or substituted or unsubstituted C₁₋₆alkyl;
   R³ is substituted or unsubstituted C₁₋₆alkyl, substituted or unsubstituted C₂₋₆alkenyl, substituted or unsubstituted C₂₋₆alkynyl, or substituted or unsubstituted C₃₋₁₀carbocyclyl;
   R⁵ is absent, hydrogen, halogen, or substituted or unsubstituted C₁₋₆alkyl;
   R⁶ is hydrogen, substituted or unsubstituted C₁₋₆alkyl, or halogen;
   R⁹ is absent or hydrogen; or
   R⁹ and R^{11a} are taken together with the carbon atoms to which they are attached to form a substituted or unsubstituted C₃₋₆carbocyclyl;
   R^{11a} is hydrogen, halogen, substituted or unsubstituted C₁₋₆alkyl, or —OR^{A11}, wherein R^{A11} is hydrogen or substituted or unsubstituted C₁₋₆alkyl;
   R^{11b} is absent or hydrogen; or
   R^{11a} and R^{11b} are joined to form an oxo (═O) group;
   R¹⁵ is hydrogen, halogen, —OH, —NH₂, substituted or unsubstituted C₁₋₆alkyl, substituted or unsubstituted C₂₋₆alkenyl, substituted or unsubstituted C₂₋₆alkynyl, substituted or unsubstituted C₁₋₆alkoxy, substituted or unsubstituted C₃₋₆carbocyclyl or substituted or unsubstituted 3- to 10-membered heterocyclyl;
   R¹⁶ is hydrogen, halogen —OH, —NH₂, substituted or unsubstituted C₁₋₆alkyl, substituted or unsubstituted C₂₋₆alkenyl, substituted or unsubstituted C₂₋₆alkynyl, substituted or unsubstituted C₁₋₆alkoxy, substituted or unsubstituted C₃₋₆carbocyclyl or substituted or unsubstituted 3- to 10-membered heterocyclyl;
   R¹⁷ is absent, hydrogen or substituted or unsubstituted C₁₋₆alkyl; provided that when the bond between C16 and C17 is a double bond, then R¹⁷ is absent;
   or R¹⁵ and R¹⁶ are taken together with the carbon atoms to which they are attached, to form a substituted or unsubstituted C₃₋₆carbocyclyl;
   or R¹⁶ and R¹⁷ are taken together with the carbon atoms to which they are attached to form a substituted or unsubstituted C₃₋₆carbocyclyl;
   provided that R¹⁵ and R¹⁶ and R¹⁶ and R¹⁷ cannot both be taken together with the carbon atoms to which they are attached, to form a substituted or unsubstituted C₃₋₆ carbocyclyl; and
   provided that at least one of R¹⁵, R¹⁶, and R¹⁷ is not hydrogen;
   R¹⁸ is hydrogen, substituted or unsubstituted C₁₋₆alkyl, OR^{A18}, wherein R^{A18} is substituted or unsubstituted C₁₋₆alkyl;
   R¹⁹ is hydrogen, substituted or unsubstituted C₁₋₆alkyl, OR^{A19}, wherein R^{A19} is substituted or unsubstituted C₁₋₆alkyl;
   X is —(C(R^{X})₂)ₙ— or —O—, wherein R^{X} is hydrogen or fluorine, or one R^{X} group and R^{23b} are joined to form a double bond;
   n is 1, 2 or 3;
   each instance of R^{23a} and R^{23b} is independently hydrogen or fluorine;
   R^{24a} is selected from the group consisting of substituted or unsubstituted C₁₋₆alkyl, substituted or unsubstituted C₂₋₆alkenyl, substituted or unsubstituted C₂₋₆ alkynyl, substituted or unsubstituted C₃₋₁₀carbocyclyl, substituted or unsubstituted 3- to 10-membered heterocyclyl, substituted or unsubstituted C₆₋₁₄ aryl, and substituted or unsubstituted 5- to 10-membered heteroaryl;
   R^{24b} is hydrogen or substituted or unsubstituted C₁₋₆alkyl;
   or R^{24a} and R^{24b}, taken together with the carbon atom to which they are attached, form a substituted or unsubstituted C₃₋₆carbocyclyl, or substituted or unsubstituted 3- to 10-membered heterocyclyl;
   q is 0, 1 or 2;
   u is 1 or 2;
   s is 1 or 2; and
   each [symbol] independently represents a single or double bond, provided if the bond between C5 and C6 is a double bond, R⁵ is absent, and if the bond between C9 and C11 is a double bond, R⁹ and R^{11b} are absent.

B-1A. A compound of Formula (B-IA):

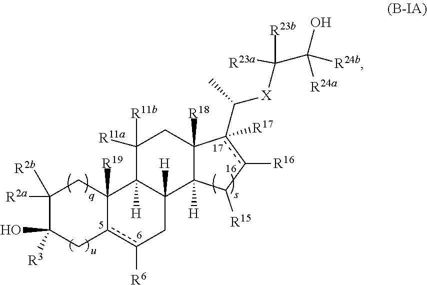

or a pharmaceutically acceptable salt thereof;
wherein:
   each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein R^{A2} is hydrogen or substituted or unsubstituted C₁₋₆alkyl;
   R³ is substituted or unsubstituted C₁₋₆alkyl, substituted or unsubstituted C₂₋₆alkenyl, substituted or unsubstituted C₂₋₆alkynyl, or substituted or unsubstituted C₃₋₁₀carbocyclyl;
   R⁶ is hydrogen, substituted or unsubstituted C₁₋₆alkyl, or halogen;
   R^{11a} is hydrogen or —OR^{A11}, wherein R^{A11} is hydrogen or substituted or unsubstituted C₁₋₆alkyl, and R^{11b} is hydrogen; or R^{11a} and R^{11b} are joined to form an oxo (═O) group;
   [symbol] represents a single or double bond; provided if the bond between C5 and C6 is a single bond, then the hydrogen at C5 is in the alpha configuration;
   R¹⁵ is hydrogen, halogen, —OH, —NH₂, substituted or unsubstituted C₁₋₆alkyl, substituted or unsubstituted C₂₋₆alkenyl, substituted or unsubstituted C₂₋₆alkynyl, substituted or unsubstituted C₁₋₆alkoxy, substituted or unsubstituted C₃₋₆carbocyclyl or substituted or unsubstituted 3- to 10-membered heterocyclyl;
   R¹⁶ is hydrogen, halogen —OH, —NH₂, substituted or unsubstituted C₁₋₆alkyl, substituted or unsubstituted C₂₋₆alkenyl, substituted or unsubstituted C₂₋₆alkynyl, substituted or unsubstituted C₁₋₆alkoxy, substituted or unsubstituted C₃₋₆carbocyclyl or substituted or unsubstituted 3- to 10-membered heterocyclyl;
   R¹⁷ is absent, hydrogen or substituted or unsubstituted C₁₋₆alkyl; provided that when the bond between C16 and C17 is a double bond, then R¹⁷ is absent;
   or R¹⁵ and R¹⁶ are taken together with the carbon atoms to which they are attached, to form a substituted or unsubstituted C₃₋₆carbocyclyl;
   or R¹⁶ and R¹⁷ are taken together with the carbon atoms to which they are attached to form a substituted or unsubstituted C₃₋₆carbocyclyl;
   provided that R¹⁵ and R¹⁶ and R¹⁶ and R¹⁷ cannot both be taken together with the carbon atoms to which they are attached, to form a substituted or unsubstituted C₃₋₆carbocyclyl; and
   provided that at least one of R¹⁵, R¹⁶, and R¹⁷ is not hydrogen;
   R¹⁸ is hydrogen, substituted or unsubstituted C₁₋₆alkyl, OR^{A18}, wherein R^{A18} is substituted or unsubstituted C₁₋₆alkyl;
   R¹⁹ is hydrogen, substituted or unsubstituted C₁₋₆alkyl, OR^{A19}, wherein R^{A19} is substituted or unsubstituted C₁₋₆alkyl;
   X is —(C(R^{X})₂)ₙ— or —O—, wherein R^{X} is hydrogen or fluorine, wherein n is 1, 2 or 3;
   each instance of R^{23a} and R^{23b} is independently hydrogen or fluorine, or one R^{X} group and R^{23b} are joined to form a double bond;
   R^{24a} is a non-hydrogen group selected from the group consisting of substituted or unsubstituted C₁₋₆alkyl, substituted or unsubstituted C₂₋₆alkenyl, substituted or unsubstituted C₂₋₆alkynyl, substituted or unsubstituted C₃₋₁₀carbocyclyl, substituted or unsubstituted 3- to 10-membered heterocyclyl, substituted or unsubstituted C₆₋₁₄ aryl, and substituted or unsubstituted 5- to 10-membered heteroaryl; and
   R^{24b} is hydrogen or a substituted or unsubstituted C₁₋₆alkyl;
   or R^{24a} and R^{24b}, taken together with the carbon atom to which they are attached, form a substituted or unsubstituted C₃₋₆carbocyclyl, or substituted or unsubstituted 3- to 10-membered heterocyclyl;
q is 0, 1 or 2;
u is 1 or 2; and
s is 1 or 2.

B-2. The compound or pharmaceutically acceptable salt thereof according to embodiment B-1, wherein the compound of formula (B-I) is a compound of formula (B-I-a):

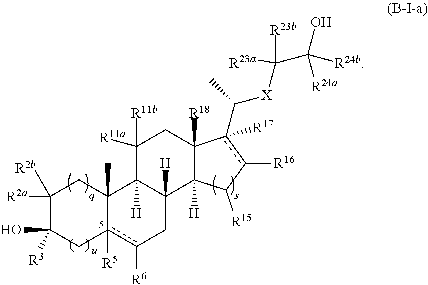

B-3. The compound or pharmaceutically acceptable salt thereof according to embodiment B-1, wherein the compound of formula (B-I) is a compound of formula (B-I-b):

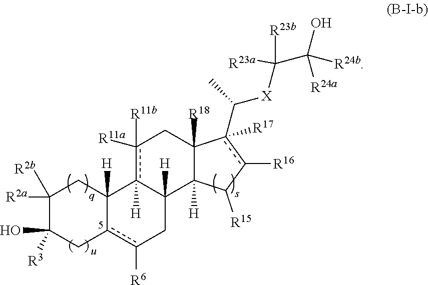

B-4. The compound or pharmaceutically acceptable salt thereof according to embodiment B-1, wherein the compound of formula (B-I) is a compound of formula (B-II):

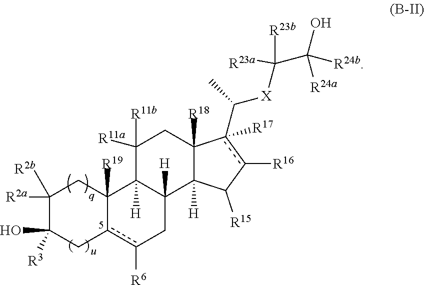

B-5. The compound or pharmaceutically acceptable salt thereof according to embodiment B-4, wherein the compound of formula (B-II) is a compound of formula (B-IIa):

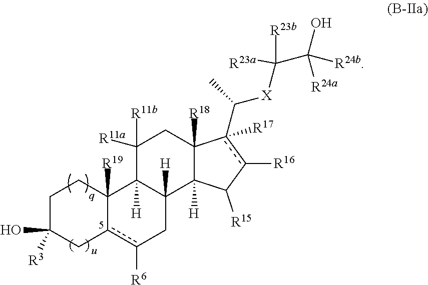

B-6. The compound or pharmaceutically acceptable salt thereof according to embodiment B-5, wherein the compound of formula (B-IIa) is a compound of formula (B-IIa-1):

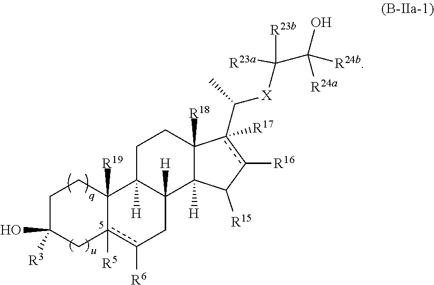

B-7. The compound or pharmaceutically acceptable salt thereof according to embodiment B-6, wherein the compound of formula (B-IIa-1) is a compound of formula (B-IIa-1a):

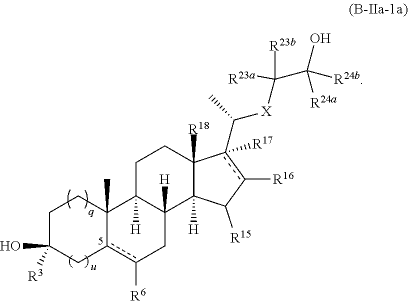

B-8. The compound or pharmaceutically acceptable salt thereof according to embodiment B-7, wherein the compound of formula (B-IIa-1a) is a compound of formula (B-IIa-1a-i):

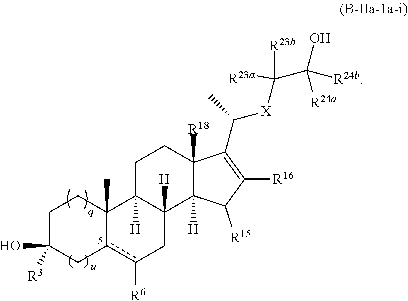

B-9. The compound or pharmaceutically acceptable salt thereof according to embodiment B-8, wherein the compound of formula (B-IIa-1a-i) is a compound of formula (B-IIa-1a-ia):

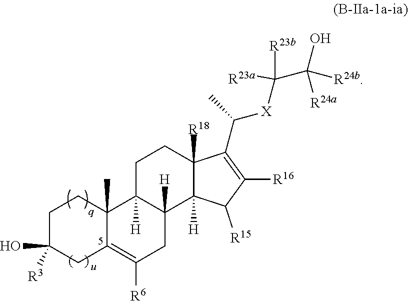

B-10. The compound or pharmaceutically acceptable salt thereof according to embodiment B-9, wherein the compound of formula (B-IIa-1a-i) is a compound of formula (B-IIa-1a-ib):

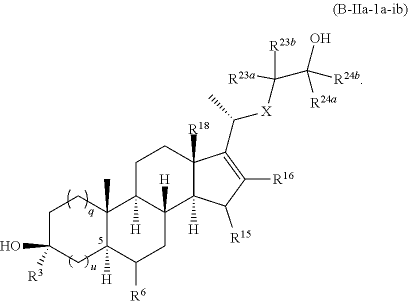

B-11. The compound or pharmaceutically acceptable salt thereof according to embodiment B-10, wherein the compound of formula (B-IIa-1a) is a compound of formula (B-IIa-1a-ii):

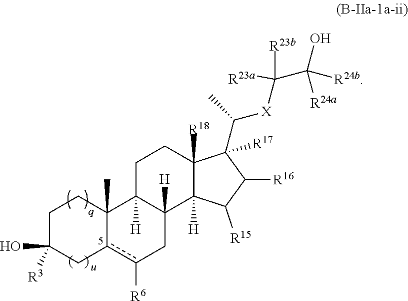

B-12. The compound or pharmaceutically acceptable salt thereof according to embodiment B-11, wherein the compound of formula (B-IIa-1a-ii) is a compound of formula (B-IIa-1a-iia):

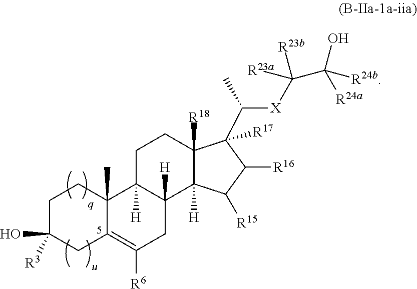

B-13. The compound or pharmaceutically acceptable salt thereof according to embodiment B-11, wherein the compound of formula (B-IIa-1a-ii) is a compound of formula (B-IIa-1a-iib):
(B-Ia-1-iib).

B-14. The compound or pharmaceutically acceptable salt thereof according to embodiment B-6, wherein the compound of formula (B-IIa-1) is a compound of formula (B-IIa-1b):

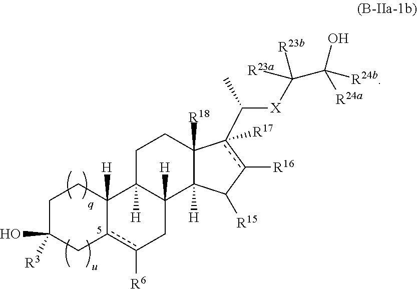

B-15. The compound or pharmaceutically acceptable salt thereof according to embodiment B-14, wherein the compound of formula (B-IIa-1b) is a compound of formula (B-IIa-1b-i):

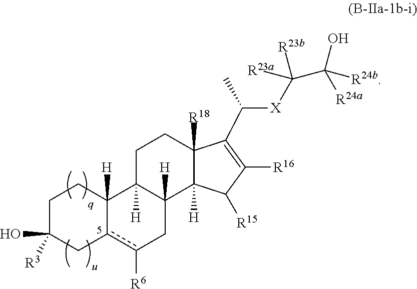

B-16. The compound or pharmaceutically acceptable salt thereof according to embodiment B-15, wherein the compound of formula (B-IIa-1b-i) is a compound of formula (B-IIa-1b-ia):

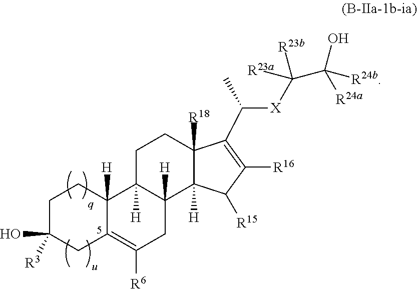

B-17. The compound or pharmaceutically acceptable salt thereof according to embodiment B-16, wherein the compound of formula (B-IIa-1b-i) is a compound of formula (B-IIa-1b-ib):

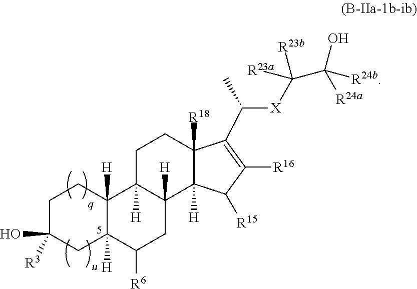

B-18. The compound or pharmaceutically acceptable salt thereof according to embodiment B-17, wherein the compound of formula (B-IIa-1b) is a compound of formula (B-IIa-1b-ii):

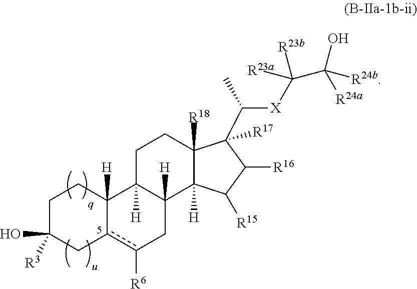

B-19. The compound or pharmaceutically acceptable salt thereof according to embodiment B-18, wherein the compound of formula (B-IIa-1b-ii) is a compound of formula (B-IIa-1b-iia):

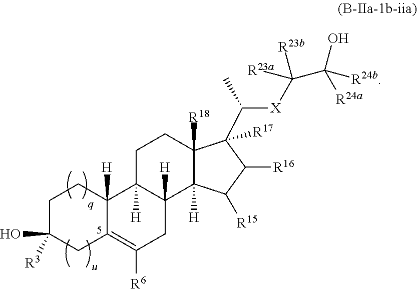

B-20. The compound or pharmaceutically acceptable salt thereof according to embodiment B-18, wherein the compound of formula (B-IIa-1a-ii) is a compound of formula (B-IIa-1a-iib):

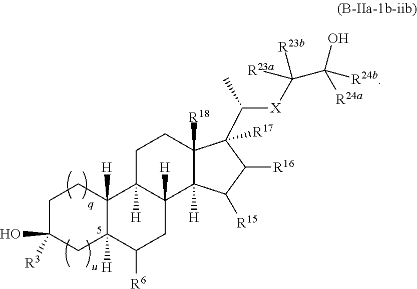

B-21. The compound or pharmaceutically acceptable salt thereof according to embodiment B-1, wherein the compound of formula (B-I) is a compound of formula (B-III):

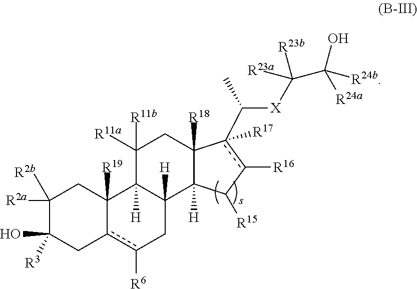

B-22. The compound or pharmaceutically acceptable salt thereof according to embodiment B-21, wherein the compound of formula (B-III) is a compound of formula (B-IIIa):

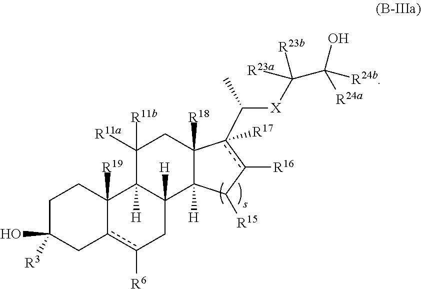

B-23. The compound or pharmaceutically acceptable salt thereof according to embodiment B-22, wherein the compound of formula (B-IIIa) is a compound of formula (B-IIIa-1):

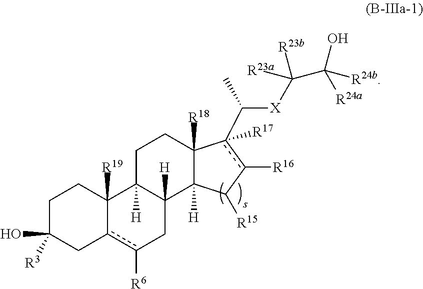

B-24. The compound or pharmaceutically acceptable salt thereof according to embodiment B-23, wherein the compound of formula (B-IIIa-1) is a compound of formula (B-IIIa-1a):

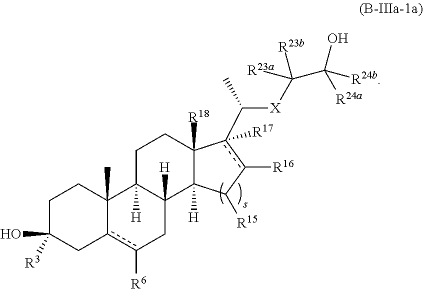

B-25. The compound or pharmaceutically acceptable salt thereof according to embodiment B-24, wherein the compound of formula (B-IIIa-1a) is a compound of formula (B-IIIa-1a-i):

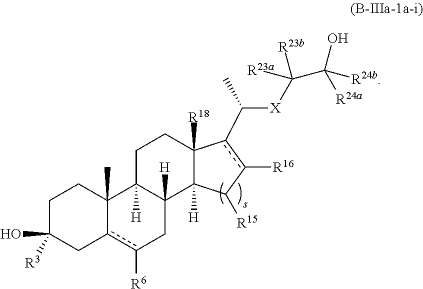

B-26. The compound or pharmaceutically acceptable salt thereof according to embodiment B-25, wherein the compound of formula (B-IIIa-1a-i) is a compound of formula (B-IIIa-1a-ia):

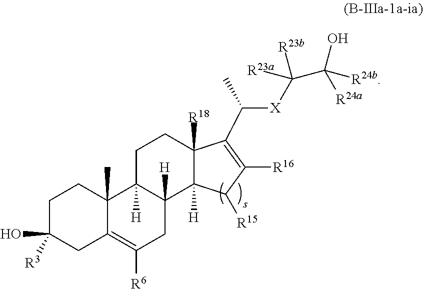

B-27. The compound or pharmaceutically acceptable salt thereof according to embodiment B-25, wherein the compound of formula (B-IIIa-1a-i) is a compound of formula (B-IIIa-1a-ib):

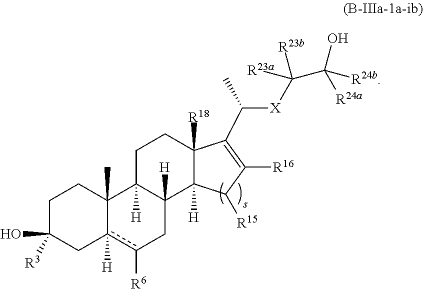

B-28. The compound or pharmaceutically acceptable salt thereof according to embodiment B-24, wherein the compound of formula (B-IIIa-1a) is a compound of formula (B-IIIa-1a-ii):

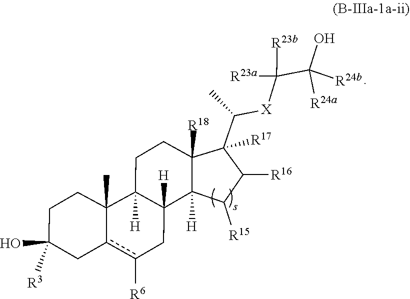

B-29. The compound or pharmaceutically acceptable salt thereof according to embodiment B-28, wherein the compound of formula (B-IIIa-1a-ii) is a compound of formula (B-IIIa-1a-iia):

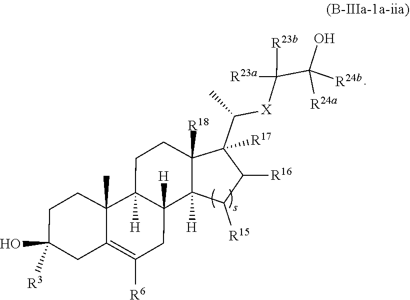

B-30. The compound or pharmaceutically acceptable salt thereof according to embodiment B-29, wherein the compound of formula (B-IIIa-1a-ii) is a compound of formula (B-IIIa-1a-iib):

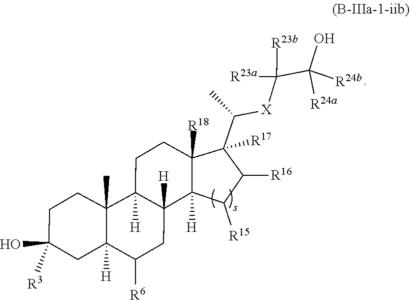

B-31. The compound or pharmaceutically acceptable salt thereof according to embodiment B-23, wherein the compound of formula (B-IIIa-1) is a compound of formula (B-IIIa-1b):

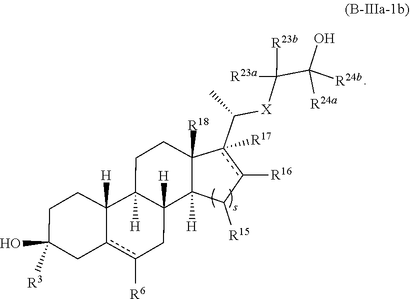

B-32. The compound or pharmaceutically acceptable salt thereof according to embodiment B-31, wherein the compound of formula (B-IIIa-1b) is a compound of formula (B-IIIa-1b-i):

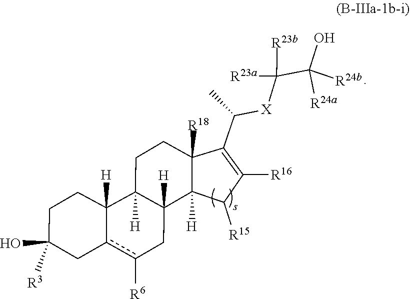

B-33. The compound or pharmaceutically acceptable salt thereof according to embodiment B-32, wherein the compound of formula (B-IIIa-1b-i) is a compound of formula (B-IIIa-1b-ia):

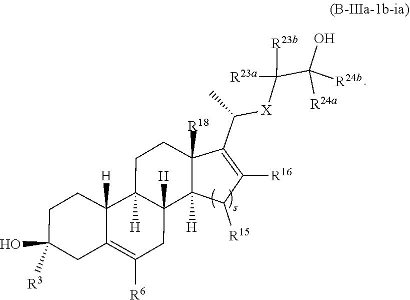

B-34. The compound or pharmaceutically acceptable salt thereof according to embodiment B-32, wherein the compound of formula (B-IIIa-1b-i) is a compound of formula (B-IIIa-1b-ib):

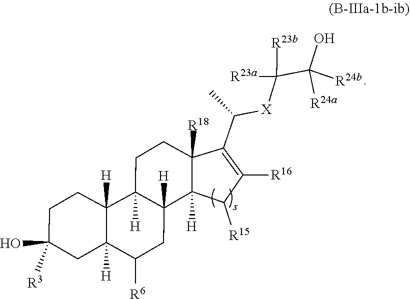

B-35. The compound or pharmaceutically acceptable salt thereof according to embodiment B-31, wherein the compound of formula (B-IIIa-1b) is a compound of formula (B-IIIa-1b-ii):

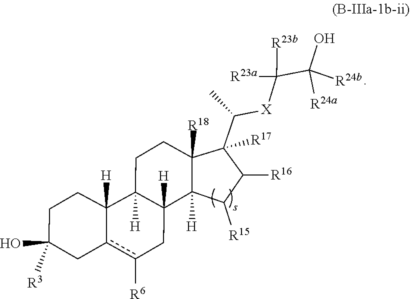

B-36. The compound or pharmaceutically acceptable salt thereof according to embodiment B-35, wherein the compound of formula (B-IIIa-1b-ii) is a compound of formula (B-IIIa-1b-iia):

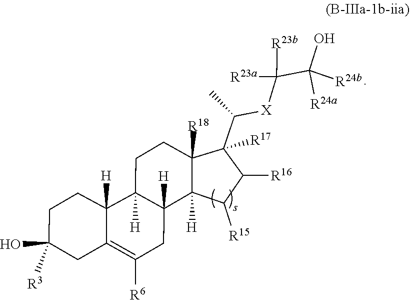

B-37. The compound or pharmaceutically acceptable salt thereof according to embodiment B-35, wherein the compound of formula (B-IIIa-1b-ii) is a compound of formula (B-IIIa-1b-iib):

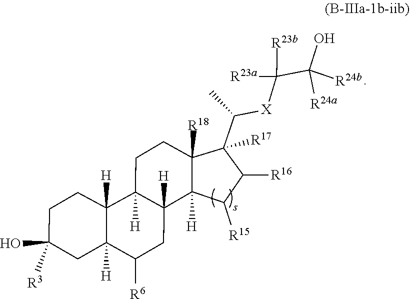

B-38. The compound or pharmaceutically acceptable salt thereof according to embodiment B-1, wherein the compound of formula (B-I) is a compound of formula (B-IV):

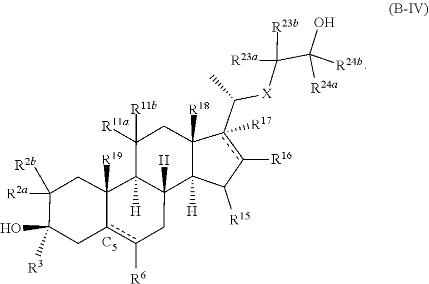

B-39. The compound or pharmaceutically acceptable salt thereof according to embodiment B-38, wherein the compound of formula (B-IV) is a compound of formula (B-IV-1):

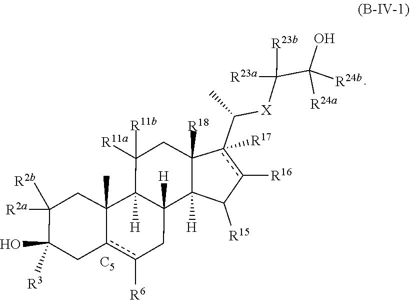

B-40. The compound or pharmaceutically acceptable salt thereof according to embodiment B-39, wherein the compound of formula (B-IV-1) is a compound of formula (B-IV-1a):

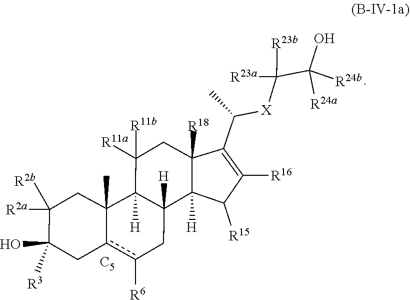

B-41. The compound or pharmaceutically acceptable salt thereof according to embodiment B-40, wherein the compound of formula (B-IV-1a) is a compound of formula (B-IV-1a-i):

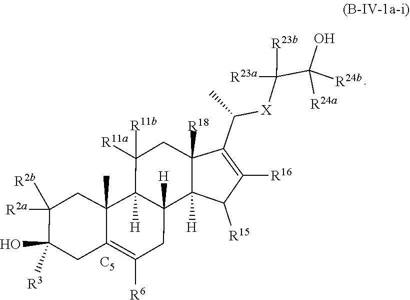

B-42. The compound or pharmaceutically acceptable salt thereof according to embodiment B-40, wherein the compound of formula (B-IV-1a) is a compound of formula (B-IV-1a-ii):

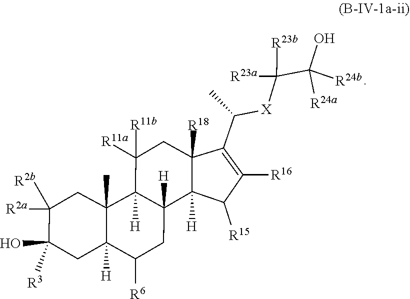

B-43. The compound or pharmaceutically acceptable salt thereof according to embodiment B-38, wherein the compound of formula (B-IV) is a compound of formula (B-IV-2):

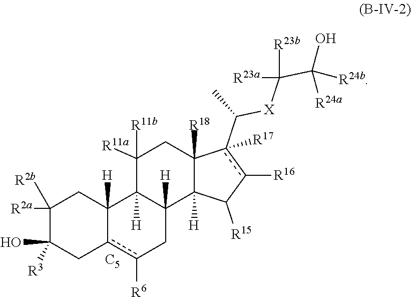

B-44. The compound or pharmaceutically acceptable salt thereof according to embodiment B-43, wherein the compound of formula (B-IV-2) is a compound of formula (B-IV-2a):

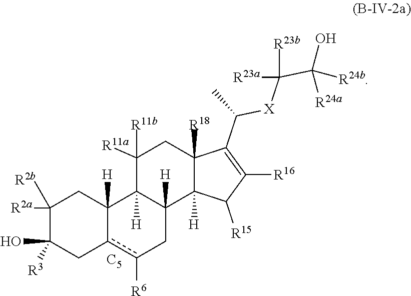

B-45. The compound or pharmaceutically acceptable salt thereof according to embodiment B-44, wherein the compound of formula (B-IV-2a) is a compound of formula (B-IV-2a-i):

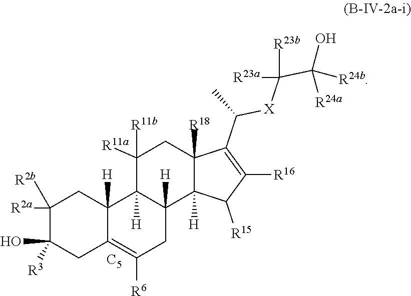

B-46. The compound or pharmaceutically acceptable salt thereof according to embodiment B-44, wherein the compound of formula (B-IV-2a) is a compound of formula (B-IV-2a-ii):

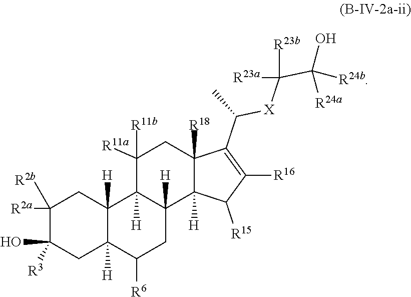

B-47. The compound or pharmaceutically acceptable salt thereof according to embodiment B-1, wherein the compound of formula (B-I) is a compound of formula (B-V):

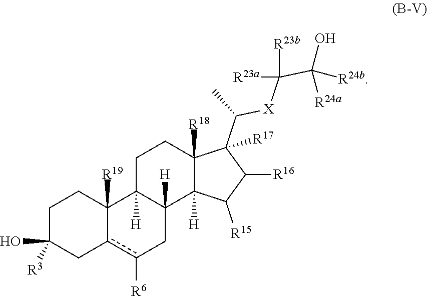

B-48. The compound or pharmaceutically acceptable salt thereof according to embodiment B-47, wherein the compound of formula (B-V) is a compound of formula (B-Va):

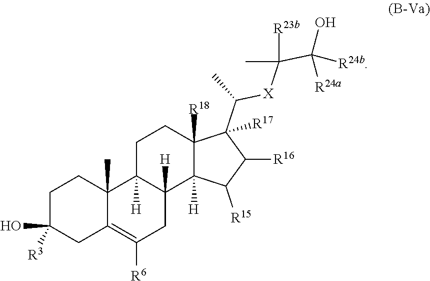

B-49. The compound or pharmaceutically acceptable salt thereof according to embodiment B-48, wherein the compound of formula (B-Va) is a compound of formula (B-Va-1):

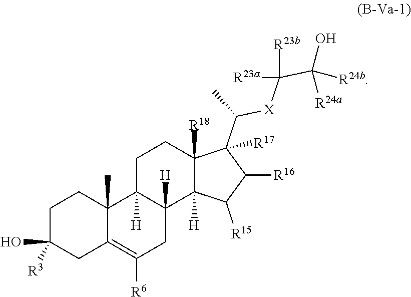

B-50. The compound or pharmaceutically acceptable salt thereof according to embodiment B-48, wherein the compound of formula (B-Va) is a compound of formula (B-Va-2):

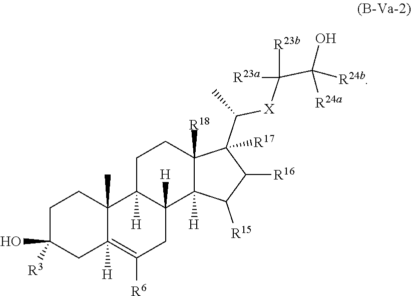

B-51. The compound or pharmaceutically acceptable salt thereof according to embodiment B-47, wherein the compound of formula (B-V) is a compound of formula (B-Vb):

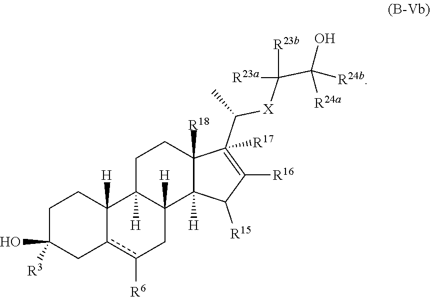

B-52. The compound or pharmaceutically acceptable salt thereof according to embodiment B-51, wherein the compound of formula (B-Vb) is a compound of formula (B-Vb-1):

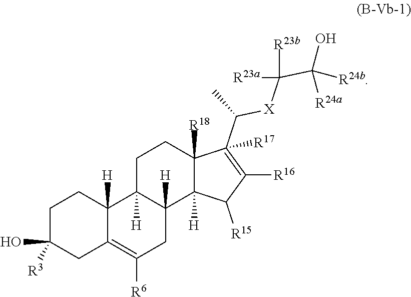

B-53. The compound or pharmaceutically acceptable salt thereof according to embodiment B-51, wherein the compound of formula (B-Vb) is a compound of formula (B-Vb-2):

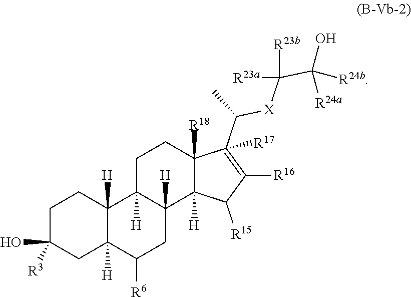

B-54. The compound or pharmaceutically acceptable salt thereof according to any one of embodiments B-1 to B-4, B-21, and B-38 to B-46, wherein each of R^{2a} and R^{2b} is independently hydrogen.

B-55. The compound or pharmaceutically acceptable salt thereof according to any one of embodiments B-1 to B-5, B-21 to B-22, or B-38 to B-46, wherein each of R^{11a} and R^{11b} is independently hydrogen.

B-56. The compound or pharmaceutically acceptable salt thereof according to any one of embodiments B-1 to B-53 wherein R³ is unsubstituted C₁₋₆alkyl.

B-57. The compound or pharmaceutically acceptable salt thereof according to embodiment B-56, wherein R³ is methyl, ethyl or propyl.

B-58. The compound or pharmaceutically acceptable salt thereof according to embodiment B-57, wherein R³ is ethyl.

B-59. The compound or pharmaceutically acceptable salt thereof according to any one of embodiments B-1 to B-53, wherein R⁶ is hydrogen.

B-60. The compound or pharmaceutically acceptable salt thereof according to any one of embodiments B-1 to B-53, wherein R¹⁵ is hydrogen, substituted or unsubstituted C₁₋₆alkyl or substituted or unsubstituted C₃₋₆carbocyclyl.

B-61. The compound or pharmaceutically acceptable salt thereof according to embodiment B-60, wherein R¹⁵ is hydrogen, unsubstituted C₁₋₆alkyl or unsubstituted C₃₋₆carbocyclyl.

B-62. The compound or pharmaceutically acceptable salt thereof according to embodiment B-61, wherein R¹⁵ is hydrogen, —CH₃ or cyclopropyl.

B-63. The compound or pharmaceutically acceptable salt thereof according to embodiment B-62, wherein R¹⁵ is hydrogen.

B-64. The compound or pharmaceutically acceptable salt thereof according to embodiment B-62, wherein R¹⁵ is —CH₃.

B-65. The compound or pharmaceutically acceptable salt thereof according to embodiment B-62, wherein R¹⁵ is cyclopropyl.

B-66. The compound or pharmaceutically acceptable salt thereof according to any one of embodiments B-1 to B-53, wherein R¹⁶ is hydrogen, substituted or unsubstituted C₁₋₆alkyl or substituted or unsubstituted C₃₋₆carbocyclyl.

B-67. The compound or pharmaceutically acceptable salt thereof according to embodiment B-66, wherein R¹⁶ is hydrogen or unsubstituted C₁₋₆alkyl.

B-68. The compound or pharmaceutically acceptable salt thereof according to embodiment B-67, wherein R¹⁶ is hydrogen.

B-69. The compound or pharmaceutically acceptable salt thereof according to embodiment B-68 wherein R¹⁶ is —CH₃.

B-70. The compound or pharmaceutically acceptable salt thereof according to any one of embodiments B-1 to B-53, wherein R¹⁵ and R¹⁶ are taken together to form a substituted or unsubstituted C₃₋₆carbocyclyl.

B-71. The compound or pharmaceutically acceptable salt thereof according to embodiment B-70, wherein R¹⁵ and R¹⁶ are taken together to form a cyclopropyl.

B-72. The compound or pharmaceutically acceptable salt thereof according to embodiments B-1 to B-7, B-11 to B-14, B-16 to B-24, B-28 to B-31, B-35 to B-39, B-43, or B-47 to B-53, wherein R¹⁷ is hydrogen.

B-73. The compound or pharmaceutically acceptable salt thereof according to embodiments B-1 to B-7, B-11 to B-14, B-18 to B-24, B-28 to B-31, B-35 to B-39, B-43, or B-47 to B-53, wherein R¹⁶ and R¹⁷ are taken together to form a substituted or unsubstituted C₃₋₆carbocyclyl.

B-74. The compound or pharmaceutically acceptable salt thereof according to embodiment B-73, wherein R¹⁶ and R¹⁷ are taken together to form a cyclopropyl.

B-75. The compound or pharmaceutically acceptable salt thereof according to any one of embodiments B-1 to B-53, wherein R¹⁸ is hydrogen, unsubstituted C₁₋₆alkyl or C₁₋₆alkyl substituted with OR^{A18}, wherein R^{A18} is substituted or unsubstituted C₁₋₆alkyl.

B-76. The compound or pharmaceutically acceptable salt thereof according to any one of embodiments B-1 to B-53, wherein R¹⁸ is hydrogen, —CH₃, CH₂OCH₃, —OCH₃, —CH₂CH₃, or —CH(CH₃)₂.

B-77. The compound or pharmaceutically acceptable salt thereof according to any one of embodiments B-1 to B-53, wherein R¹⁸ is —CH₃.

B-78. The compound or pharmaceutically acceptable salt thereof according to any one of embodiments B-1, B-4 to B-6, B-21 to B-23, B-38, and B-47, wherein R¹⁹ is hydrogen, unsubstituted C₁₋₆alkyl or C₁₋₆alkyl substituted with OR^{A19}, wherein R^{A19} is substituted or unsubstituted C₁₋₆alkyl.

B-79. The compound or pharmaceutically acceptable salt thereof according to embodiment B-78, wherein R¹⁹ is hydrogen, —CH₃, —CH₂OCH₃, —OCH₃, —CH₂CH₃, or CH(CH₃)₂.

B-80. The compound or pharmaceutically acceptable salt thereof according to embodiment B-79, wherein R¹⁹ is hydrogen, —CH₃, —CH₂CH₃.

B-81. The compound or pharmaceutically acceptable salt thereof according to any one of embodiments B-1 to B-53, wherein X is —(C(R^{X})₂)ₙ—, wherein R^{X} is hydrogen or fluorine, and n is 1 or 2.

B-82. The compound or pharmaceutically acceptable salt thereof according to embodiment B-81, wherein X is —CH₂— or —(CH₂)₂—.

B-83. The compound or pharmaceutically acceptable salt thereof according to embodiment B-82, wherein X is —CH₂—.

B-84. The compound or pharmaceutically acceptable salt thereof according to embodiment B-82, wherein X is —(CH₂)₂—.

B-85. The compound or pharmaceutically acceptable salt thereof according to any one of embodiments B-1 to B-53, wherein each of R^{23a} and R^{23b} is independently hydrogen.

B-86. The compound or pharmaceutically acceptable salt thereof according to any one of embodiments B-1 to B-53, wherein each of R^{23a} and R^{23b} is independently fluorine.

B-87. The compound or pharmaceutically acceptable salt thereof according to any one of embodiments B-1 to B-53, wherein R^{24a} is selected from substituted or unsubstituted C₁₋₆alkyl.

B-88. The compound or pharmaceutically acceptable salt thereof according to embodiment B-87, wherein R^{24a} is methyl, isopropyl or —CF₃.

B-89. The compound or pharmaceutically acceptable salt thereof according to embodiment B-88, wherein R^{24a} is methyl.

B-90. The compound or pharmaceutically acceptable salt thereof according to embodiment B-88, wherein R^{24a} is isopropyl.

B-91. The compound or pharmaceutically acceptable salt thereof according to embodiment B-88, wherein R^{24a} is —CF₃.

B-92. The compound or pharmaceutically acceptable salt thereof according to any one of embodiments B-1 to B-53, wherein R^{24b} is hydrogen or unsubstituted C₁₋₆alkyl.

B-93. The compound or pharmaceutically acceptable salt thereof according to embodiment B-92, wherein R^{24b} is hydrogen or methyl.

B-94. The compound or pharmaceutically acceptable salt thereof of embodiment B-1, wherein the compound of formula (B-I) is selected from the group consisting of Compounds B-1-B-24 and pharmaceutically acceptable salts thereof.

B-95. The compound or pharmaceutically acceptable salt thereof according to embodiment B-1, wherein the compound of formula (B-I) is selected from the group consisting of Compounds B-1, B-3, B-4, B-10, B-11, B-12, B-14, B-15, B-18, B-20, B-23, and pharmaceutically acceptable salts thereof.

B-96. The compound or pharmaceutically acceptable salt thereof according to embodiment B-1, wherein the compound of formula (B-I) is selected from the group consisting of Compounds B-1, B-2, B-3, B-4, B-5, B-6, B-13, B-14, B-22, B-23, B-24, and pharmaceutically acceptable salts thereof.

B-97. A pharmaceutical composition comprising a compound or pharmaceutically acceptable salt thereof according to any one of embodiments B-1 to B-96, and a pharmaceutically acceptable carrier.

B-98. A method for treating a CNS-related condition in a subject, comprising administering to the subject an effective amount of a compound or pharmaceutically acceptable salt thereof according to any one of embodiments B-1 to B-96, or a pharmaceutical composition according to embodiment B-97.

B-99. The method according to embodiment B-98, wherein the CNS-related condition is selected from the group consisting of an adjustment disorder, anxiety disorder (including obsessive-compulsive disorder, post-traumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease) and tinnitus.

B-100. A method of inducing sedation or anesthesia in a subject, comprising administering to the subject an effective amount of a compound or pharmaceutically acceptable salt thereof according to any one of embodiments B-1 to B-96, or a pharmaceutical composition according to embodiment B-97.

B-101. A compound or pharmaceutically acceptable salt thereof according to any one of embodiments B-1 to B-96, or a pharmaceutical composition according to embodiment B-97, for use in treating a CNS-related condition in a subject.

B-102. The compound or pharmaceutically acceptable salt thereof, or pharmaceutical composition for use according to embodiment B-101, wherein the CNS-related condition is selected from the group consisting of an adjustment disorder, anxiety disorder (including obsessive-compulsive disorder, post-traumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease) and tinnitus.

B-103. A compound or pharmaceutically acceptable salt thereof according to any one of embodiments B-1 to B-96, or a pharmaceutical composition according to embodiment B-97, for use inducing sedation or anesthesia in a subject.

B-104. Use of a compound or pharmaceutically acceptable salt thereof according to any one of embodiments B-1 to B-96, or a pharmaceutical composition according to embodiment B-97, for the manufacture of a medicament for treating a CNS-related condition in a subject.

B-105. The use according to embodiment B-104, wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease) and tinnitus.

B-106. Use of a compound or pharmaceutically acceptable salt thereof according to any one of embodiments B-1 to B-96, or a pharmaceutical composition according to embodiment B-97, for the manufacture of a medicament for inducing sedation or anesthesia in a subject.

B-107. A compound of Formula (B-I):

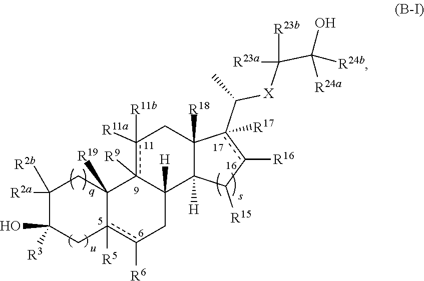

wherein:
   each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein R^{A2} is hydrogen or substituted or unsubstituted C₁₋₆alkyl;
   R³ is substituted or unsubstituted C₁₋₆alkyl, substituted or unsubstituted C₂₋₆alkenyl, substituted or unsubstituted C₂₋₆alkynyl, or substituted or unsubstituted C₃₋₁₀carbocyclyl;
   R⁵ is absent, hydrogen, halogen, or substituted or unsubstituted C₁₋₆alkyl;
   R⁶ is hydrogen, substituted or unsubstituted C₁₋₆alkyl, or halogen;
   R⁹ is absent or hydrogen; or
   R⁹ and R^{11a} are taken together with the carbon atoms to which they are attached to form a substituted or unsubstituted C₃₋₆carbocyclyl;
   R^{11a} is hydrogen, halogen, substituted or unsubstituted C₁₋₆alkyl, or —OR^{A11}, wherein R^{A11} is hydrogen or substituted or unsubstituted C₁₋₆alkyl;
   R^{11b} is absent or hydrogen; or
   R^{11a} and R^{11b} are joined to form an oxo (═O) group;
   R¹⁵ is hydrogen, halogen, —OH, —NH₂, substituted or unsubstituted C₁₋₆alkyl, substituted or unsubstituted C₂₋₆alkenyl, substituted or unsubstituted C₂₋₆alkynyl, substituted or unsubstituted C₁₋₆alkoxy, substituted or unsubstituted C₃₋₆carbocyclyl or substituted or unsubstituted 3- to 10-membered heterocyclyl;
   R¹⁶ is hydrogen, halogen —OH, —NH₂, substituted or unsubstituted C₁₋₆alkyl, substituted or unsubstituted C₂₋₆alkenyl, substituted or unsubstituted C₂₋₆alkynyl, substituted or unsubstituted C₁₋₆alkoxy, substituted or unsubstituted C₃₋₆carbocyclyl or substituted or unsubstituted 3- to 10-membered heterocyclyl;
   R¹⁷ is absent, hydrogen or substituted or unsubstituted C₁₋₆alkyl; provided that when the bond between C16 and C17 is a double bond, then R¹⁷ is absent;
   or R¹⁵ and R¹⁶ are taken together with the carbon atoms to which they are attached, to form a substituted or unsubstituted C₃₋₆carbocyclyl;
   or R¹⁶ and R¹⁷ are taken together with the carbon atoms to which they are attached to form a substituted or unsubstituted C₃₋₆carbocyclyl;
   provided that R¹⁵ and R¹⁶ and R¹⁶ and R¹⁷ cannot both be taken together with the carbon atoms to which they are attached, to form a substituted or unsubstituted C₃₋₆carbocyclyl; and
   provided that at least one of R¹⁵, R¹⁶, and R¹⁷ is not hydrogen;
   R¹⁸ is hydrogen, substituted or unsubstituted C₁₋₆alkyl, OR^{A18}, wherein R^{A18} is substituted or unsubstituted C₁₋₆alkyl;
   R¹⁹ is hydrogen, substituted or unsubstituted C₁₋₆alkyl, OR^{A19}, wherein R^{A19} is substituted or unsubstituted C₁₋₆alkyl;
   X is —(C(R^{X})₂)ₙ— or —O—, wherein R^{X} is hydrogen or fluorine, or one R^{X} group and R^{23b} are joined to form a double bond;
   n is 1, 2 or 3;
   each instance of R^{23a} and R^{23b} is independently hydrogen or fluorine;
   R^{24a} is selected from the group consisting of substituted or unsubstituted C₁₋₆alkyl, substituted or unsubstituted C₂₋₆alkenyl, substituted or unsubstituted C₂₋₆ alkynyl, substituted or unsubstituted C₃₋₁₀carbocyclyl, substituted or unsubstituted 3- to 10-membered heterocyclyl, substituted or unsubstituted C₆₋₁₄ aryl, and substituted or unsubstituted 5- to 10-membered heteroaryl;
   R^{24b} is hydrogen or substituted or unsubstituted C₁₋₆alkyl;
   or R^{24a} and R^{24b}, taken together with the carbon atom to which they are attached, form a substituted or unsubstituted C₃₋₆carbocyclyl, or substituted or unsubstituted 3- to 10-membered heterocyclyl;
   q is 0, 1 or 2;
   u is 1 or 2;
   s is 1 or 2; and
      each [symbol] independently represents a single or double bond, provided if the bond between C5 and C6 is a double bond, R⁵ is absent, and if the bond between C9 and C11 is a double bond, R⁹ and R^{11b} are absent.

B-107A. A compound of Formula (B-IA):

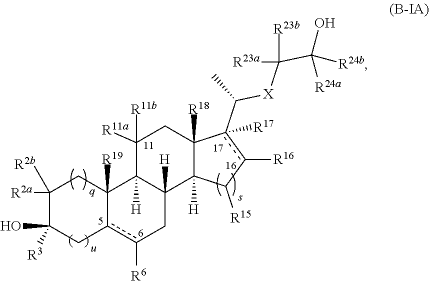

wherein:
   each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein R^{A2} is hydrogen or substituted or unsubstituted C₁₋₆alkyl;
   R³ is substituted or unsubstituted C₁₋₆alkyl, substituted or unsubstituted C₂₋₆alkenyl, substituted or unsubstituted C₂₋₆alkynyl, or substituted or unsubstituted C₃₋₁₀carbocyclyl;
   R⁶ is hydrogen, substituted or unsubstituted C₁₋₆alkyl, or halogen;
   R^{11a} is hydrogen or —OR^{A11}, wherein R^{A11} is hydrogen or substituted or unsubstituted C₁₋₆alkyl, and R^{11b} is hydrogen; or R^{11a} and R^{11b} are joined to form an oxo (═O) group;
   [symbol] represents a single or double bond; provided if the bond between C5 and C6 is a single bond, then the hydrogen at C5 is in the alpha configuration;
   R¹⁵ is hydrogen, halogen, —OH, —NH₂, substituted or unsubstituted C₁₋₆alkyl, substituted or unsubstituted C₂₋₆alkenyl, substituted or unsubstituted C₂₋₆alkynyl, substituted or unsubstituted C₁₋₆alkoxy, substituted or unsubstituted C₃₋₆carbocyclyl or substituted or unsubstituted 3- to 10-membered heterocyclyl;
   R¹⁶ is hydrogen, halogen —OH, —NH₂, substituted or unsubstituted C₁₋₆alkyl, substituted or unsubstituted C₂₋₆alkenyl, substituted or unsubstituted C₂₋₆alkynyl, substituted or unsubstituted C₁₋₆alkoxy, substituted or unsubstituted C₃₋₆carbocyclyl or substituted or unsubstituted 3- to 10-membered heterocyclyl;
   R¹⁷ is absent, hydrogen or substituted or unsubstituted C₁₋₆alkyl; provided that when the bond between C16 and C17 is a double bond, then R¹⁷ is absent;
   or R¹⁵ and R¹⁶ are taken together with the carbon atoms to which they are attached, to form a substituted or unsubstituted C₃₋₆carbocyclyl;
   or R¹⁶ and R¹⁷ are taken together with the carbon atoms to which they are attached to form a substituted or unsubstituted C₃₋₆carbocyclyl;
   provided that R¹⁵ and R¹⁶ and R¹⁶ and R¹⁷ cannot both be taken together with the carbon atoms to which they are attached, to form a substituted or unsubstituted C₃₋₆carbocyclyl; and
   provided that at least one of R¹⁵, R¹⁶, and R¹⁷ is not hydrogen;
   R¹⁸ is hydrogen, substituted or unsubstituted C₁₋₆alkyl, OR^{A18}, wherein R^{A18} is substituted or unsubstituted C₁₋₆alkyl;
   R¹⁹ is hydrogen, substituted or unsubstituted C₁₋₆alkyl, OR^{A19}, wherein R^{A19} is substituted or unsubstituted C₁₋₆alkyl;
   X is —(C(R^{X})₂)ₙ— or —O—, wherein R^{X} is hydrogen or fluorine, wherein n is 1, 2 or 3;
   each instance of R^{23a} and R^{23b} is independently hydrogen or fluorine, or one R^{X} group and R^{23b} are joined to form a double bond;
   R^{24a} is a non-hydrogen group selected from the group consisting of substituted or unsubstituted C₁₋₆alkyl, substituted or unsubstituted C₂₋₆alkenyl, substituted or unsubstituted C₂₋₆alkynyl, substituted or unsubstituted C₃₋₁₀carbocyclyl, substituted or unsubstituted 3- to 10-membered heterocyclyl, substituted or unsubstituted C₆₋₁₄ aryl, and substituted or unsubstituted 5- to 10-membered heteroaryl;
   R^{24b} is hydrogen or a substituted or unsubstituted C₁₋₆alkyl;
   or R^{24a} and R^{24b}, taken together with the carbon atom to which they are attached, form a substituted or unsubstituted C₃₋₆carbocyclyl, or substituted or unsubstituted 3- to 10-membered heterocyclyl;
   q is 0, 1 or 2;
   u is 1 or 2; and
   s is 1 or 2.

B-108. The compound according to embodiment B-107, wherein the compound of formula (B-I) is a compound of formula (B-I-a):

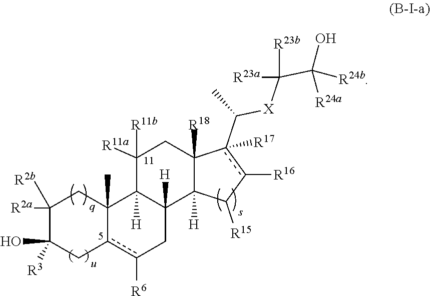

B-109. The compound according to embodiment B-107, wherein the compound of formula (B-I) is a compound of formula (B-I-b):

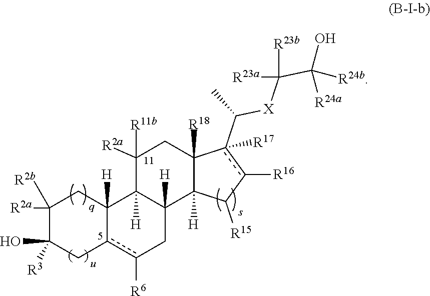

B-110. The compound according to embodiment B-107, wherein the compound of formula (B-I) is a compound of formula (B-II):

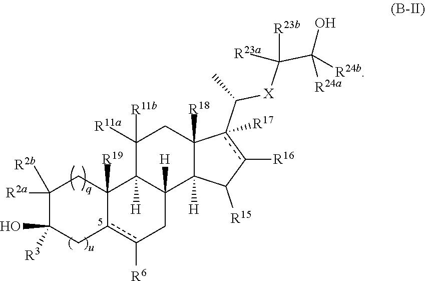

B-111. The compound according to embodiment B-110, wherein the compound of formula (B-II) is a compound of formula (B-IIa):

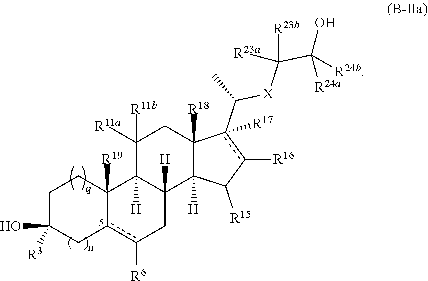

B-112. The compound according to embodiment B-111, wherein the compound of formula (B-IIa) is a compound of formula (B-IIa-1):

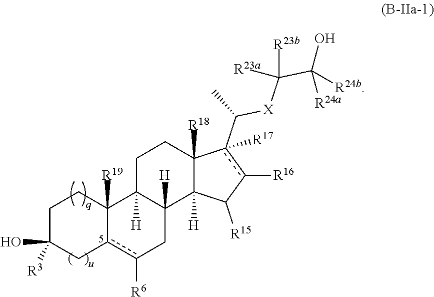

B-113. The compound according to embodiment B-112, wherein the compound of formula (B-IIa-1) is a compound of formula (B-Ia-1a):

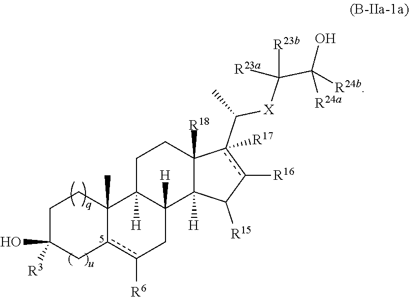

B-114. The compound according to embodiment B-113, wherein the compound of formula (B-IIa-1a) is a compound of formula (B-IIa-1a-i):

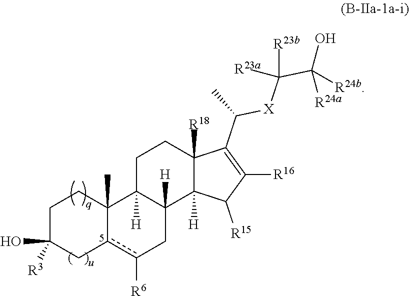

B-115. The compound according to embodiment B-114, wherein the compound of formula (B-IIa-1a-i) is a compound of formula (B-IIa-1a-ia):

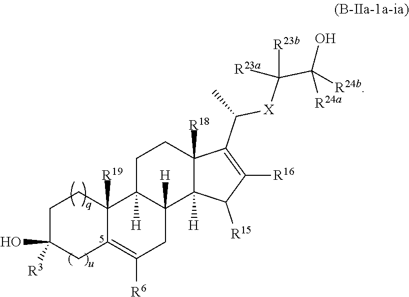

B-116. The compound according to embodiment B-114, wherein the compound of formula (B-IIa-1a-i) is a compound of formula (B-IIa-1a-ib):

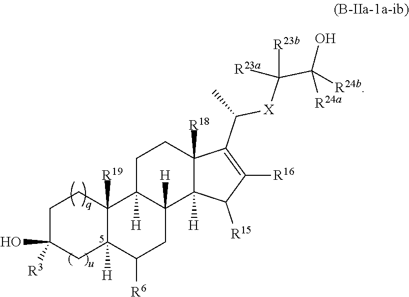

B-117. The compound according to embodiment B-113, wherein the compound of formula (B-IIa-1a) is a compound of formula (B-Ia-la-ii):

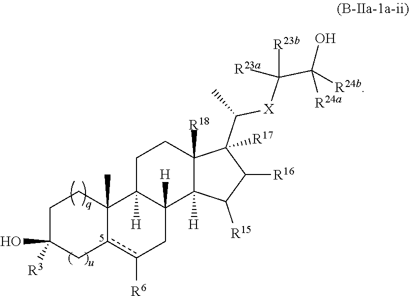

B-118. The compound according to embodiment B-117, wherein the compound of formula (B-IIa-1a-ii) is a compound of formula (B-IIa-1a-iia):

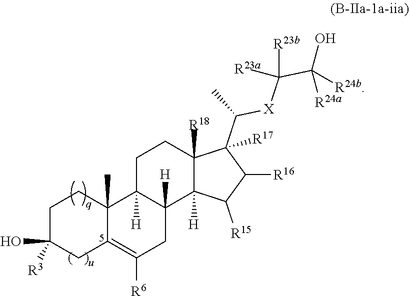

B-119. The compound according to embodiment B-118, wherein the compound of formula (B-IIa-1a-ii) is a compound of formula (B-IIa-1a-iib):

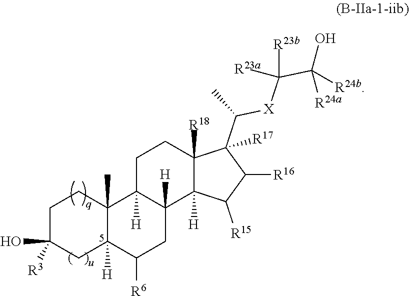

B-120. The compound according to embodiment B-112, wherein the compound of formula (B-IIa-1) is a compound of formula (B-IIa-1b):

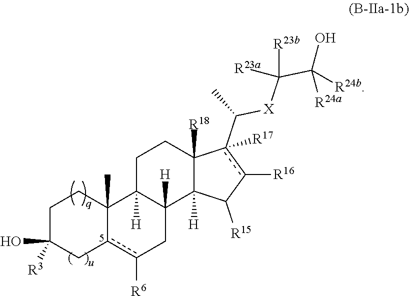

B-121. The compound according to embodiment B-120, wherein the compound of formula (B-IIa-1b) is a compound of formula (B-IIa-1b-i):

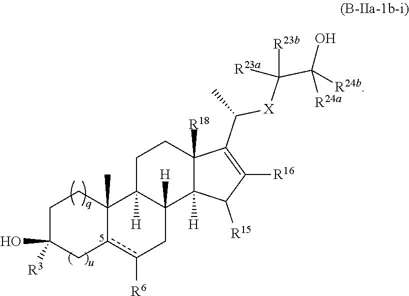

B-122. The compound according to embodiment B-121, wherein the compound of formula (B-IIa-1b-i) is a compound of formula (B-IIa-1b-ia):

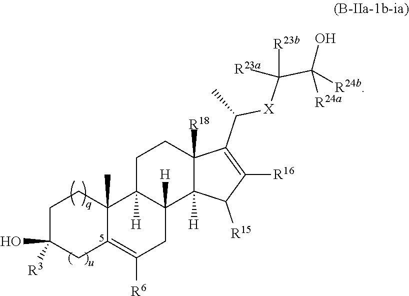

B-123. The compound according to embodiment B-121, wherein the compound of formula (B-IIa-1b-i) is a compound of formula (B-IIa-1b-ib):

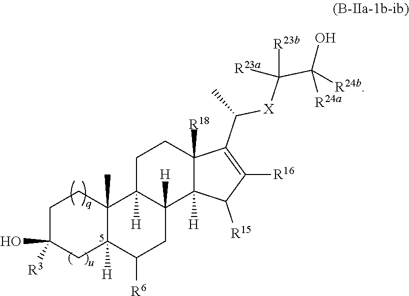

B-124. The compound according to embodiment B-120, wherein the compound of formula (B-IIa-1b) is a compound of formula (B-IIa-1b-ii):

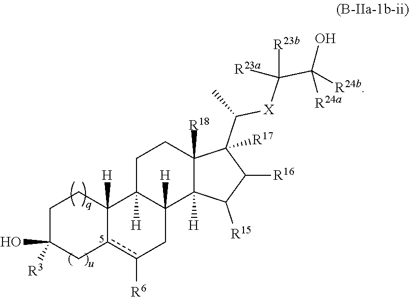

B-125. The compound according to embodiment B-124, wherein the compound of formula (B-IIa-1b-ii) is a compound of formula (B-IIa-1b-iia):

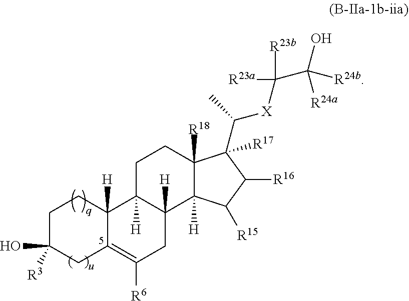

B-126. The compound according to embodiment B-125, wherein the compound of formula (B-IIa-1b-ii) is a compound of formula (B-IIa-1b-iib):

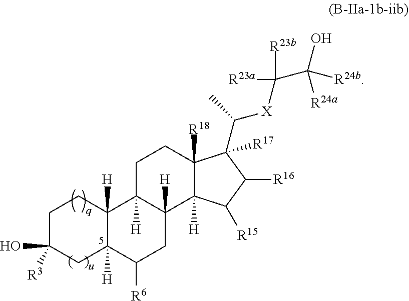

B-127. The compound according to embodiment B-107, wherein the compound of formula (B-I) is a compound of formula (B-III):

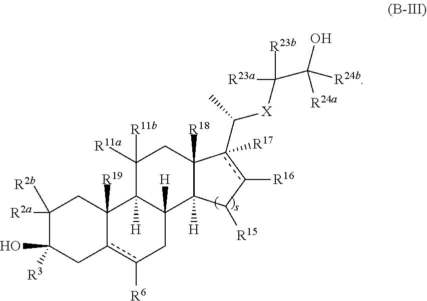

B-128. The compound according to embodiment B-127, wherein the compound of formula (B-III) is a compound of formula (B-IIIa):

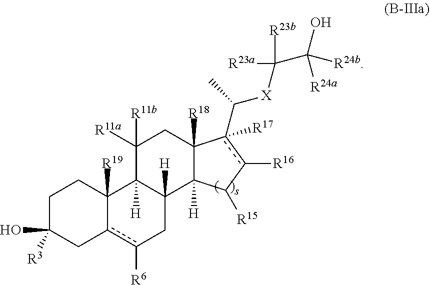

B-129. The compound according to embodiment B-128, wherein the compound of formula (B-IIIa) is a compound of formula (B-IIIa-1):

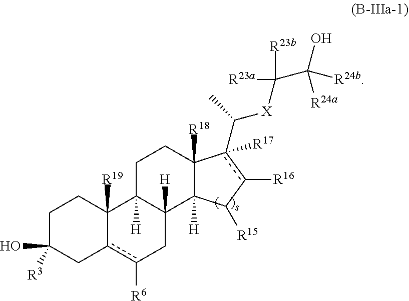

B-130. The compound according to embodiment B-129, wherein the compound of formula (B-IIIa-1) is a compound of formula (B-IIIa-1a):

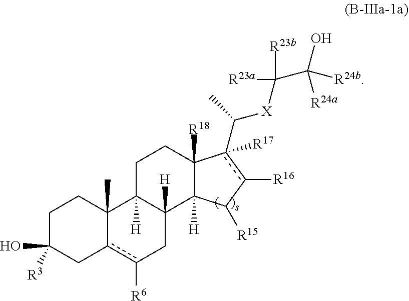

B-131. The compound according to embodiment B-130, wherein the compound of formula (B-IIIa-1a) is a compound of formula (B-IIIa-1a-i):

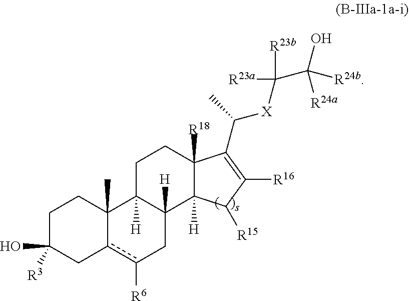

B-132. The compound according to embodiment B-131, wherein the compound of formula (B-IIIa-1a-i) is a compound of formula (B-IIIa-1a-ia):

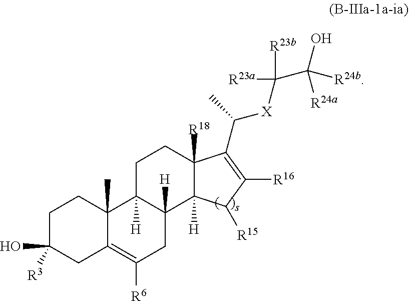

B-133. The compound according to embodiment B-131, wherein the compound of formula (B-IIIa-1a-i) is a compound of formula (B-IIIa-1a-ib):

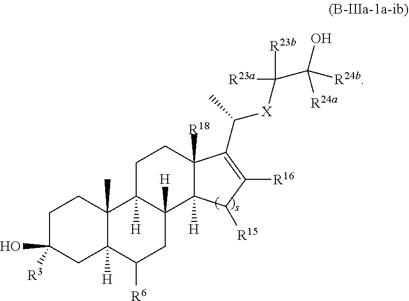

B-134. The compound according to embodiment B-130, wherein the compound of formula (B-IIIa-1a) is a compound of formula (B-IIIa-1a-ii):

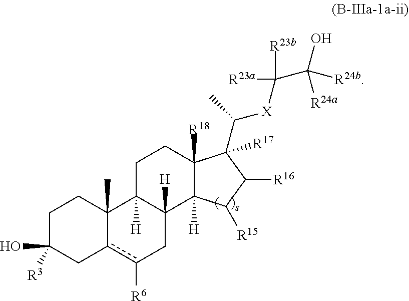

B-135. The compound according to embodiment B-134, wherein the compound of formula (B-IIIa-1a-ii) is a compound of formula (B-IIIa-1a-iia):

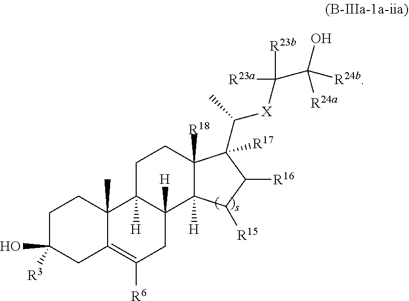

B-136. The compound according to embodiment B-134, wherein the compound of formula (B-IIIa-1a-ii) is a compound of formula (B-IIIa-1a-iib):

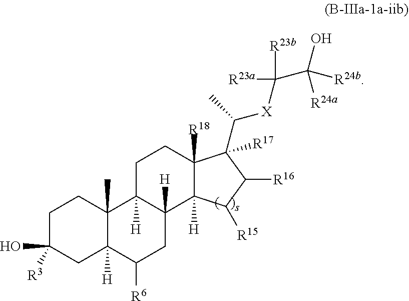

B-137. The compound according to embodiment B-129, wherein the compound of formula (B-IIIa-1) is a compound of formula (B-IIIa-1b):

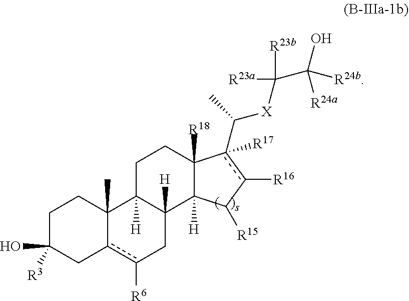

B-138. The compound according to embodiment B-137, wherein the compound of formula (B-IIIa-1b) is a compound of formula (B-IIIa-1b-i):

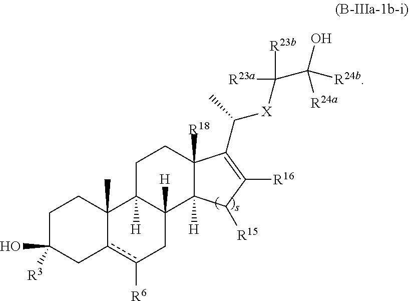

B-139. The compound according to embodiment B-138, wherein the compound of formula (B-IIIa-1b-i) is a compound of formula (B-IIIa-1b-ia):

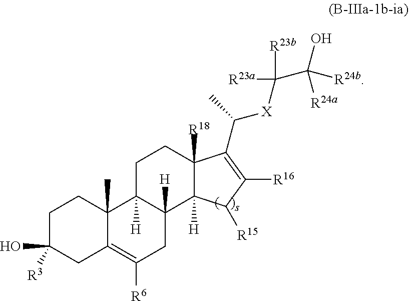

B-140. The compound according to embodiment B-138, wherein the compound of formula (B-IIIa-1b-i) is a compound of formula (B-IIIa-1b-ib):

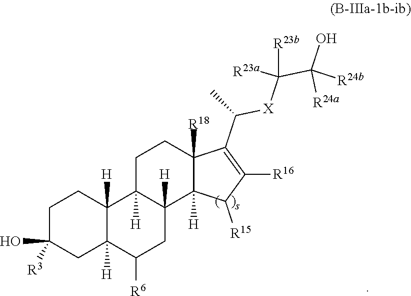

B-141. The compound according to embodiment B-137, wherein the compound of formula (B-IIIa-1b) is a compound of formula (B-IIIa-1b-ii):

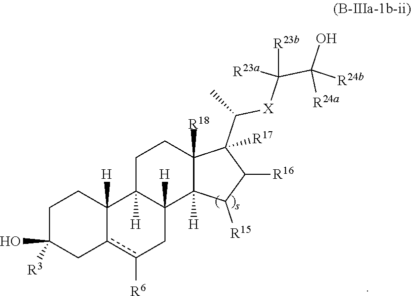

B-142. The compound according to embodiment B-141, wherein the compound of formula (B-IIIa-1b-ii) is a compound of formula (B-IIIa-1b-iia):

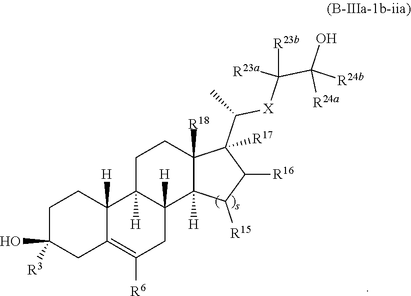

B-143. The compound according to embodiment B-141, wherein the compound of formula (B-IIIa-1b-ii) is a compound of formula (B-IIIa-1b-iib):

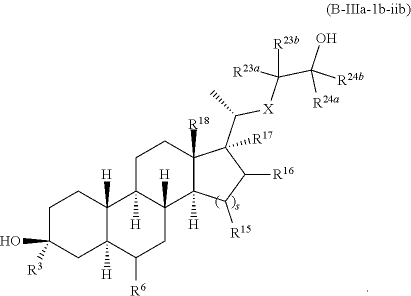

B-144. The compound according to embodiment B-107, wherein the compound of formula (B-I) is a compound of formula (B-IV):

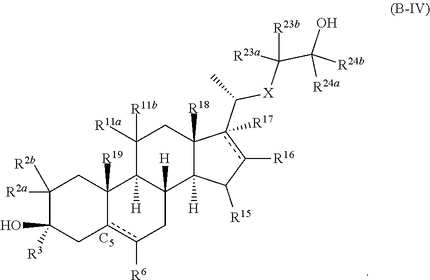

B-145. The compound according to embodiment B-144, wherein the compound of formula (B-IV) is a compound of formula (B-IV-1):

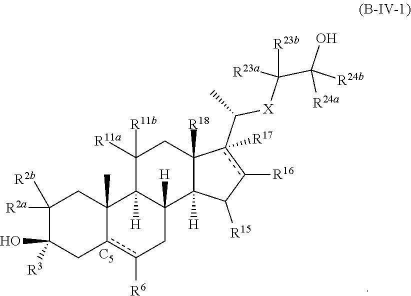

B-146. The compound according to embodiment B-145, wherein the compound of formula (B-IV-1) is a compound of formula (B-IV-1a):

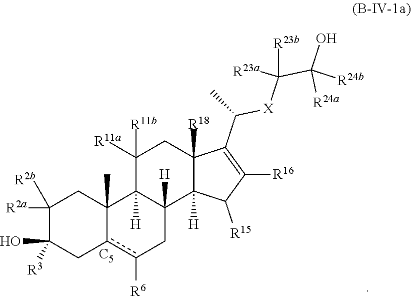

B-147. The compound according to embodiment B-146, wherein the compound of formula (B-IV-1a) is a compound of formula (B-IV-1a-i):

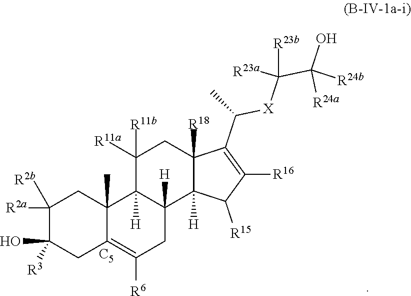

B-148. The compound according to embodiment B-146, wherein the compound of formula (B-IV-1a) is a compound of formula (B-IV-1a-ii):

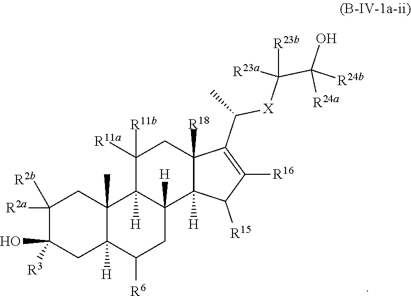

B-149. The compound according to embodiment B-144, wherein the compound of formula (B-IV) is a compound of formula (B-IV-2):

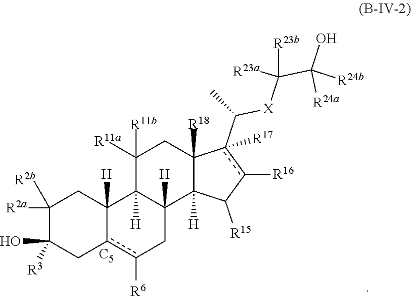

B-150. The compound according to embodiment B-149, wherein the compound of formula (B-IV-2) is a compound of formula (B-IV-2a):

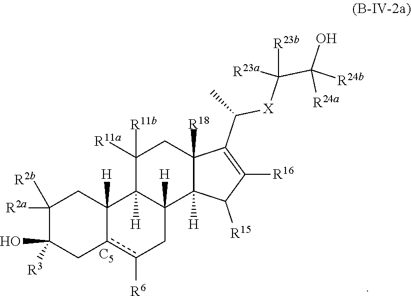

B-151. The compound according to embodiment B-150, wherein the compound of formula (B-IV-2a) is a compound of formula (B-IV-2a-i):

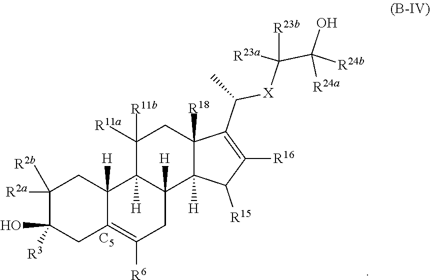

B-152. The compound according to embodiment B-150, wherein the compound of formula (B-IV-2a) is a compound of formula (B-IV-2a-ii):

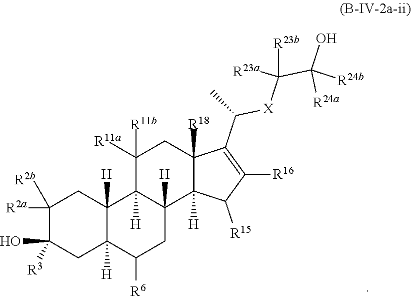

B-153. The compound according to embodiment B-107, wherein the compound of formula (B-I) is a compound of formula (B-V):

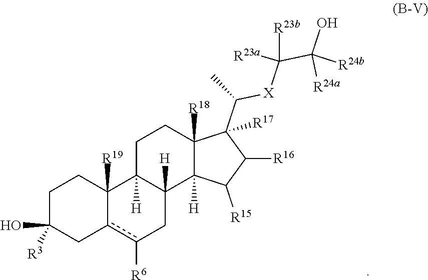

B-154. The compound according to embodiment B-153, wherein the compound of formula (B-V) is a compound of formula (B-Va):

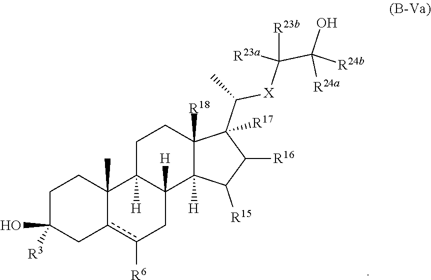

B-155. The compound according to embodiment B-154, wherein the compound of formula (B-Va) is a compound of formula (B-Va-1):

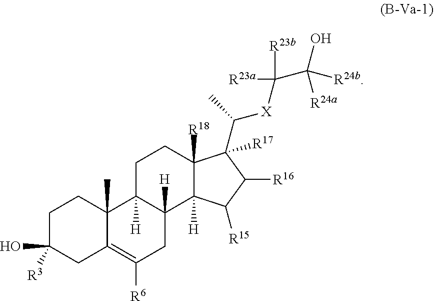

B-156. The compound according to embodiment B-155, wherein the compound of formula (B-Va) is a compound of formula (B-Va-2):

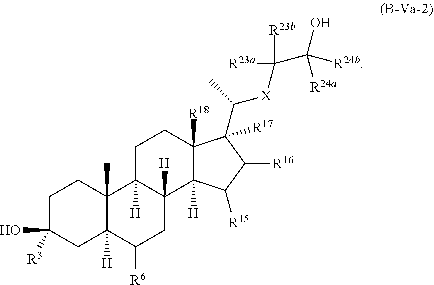

B-157. The compound according to embodiment B-153, wherein the compound of formula (B-V) is a compound of formula (B-Vb):

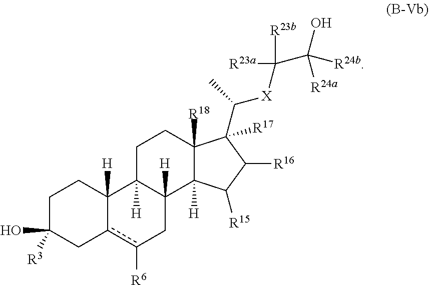

B-158. The compound according to embodiment B-157, wherein the compound of formula (B-Vb) is a compound of formula (B-Vb-1):

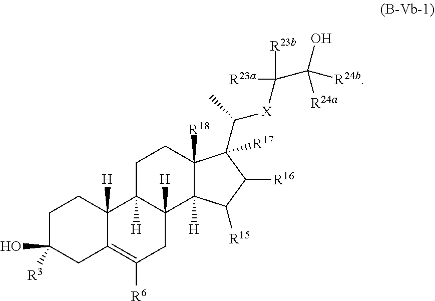

B-159. The compound according to embodiment B-157, wherein the compound of formula (B-Vb) is a compound of formula (B-Vb-2):

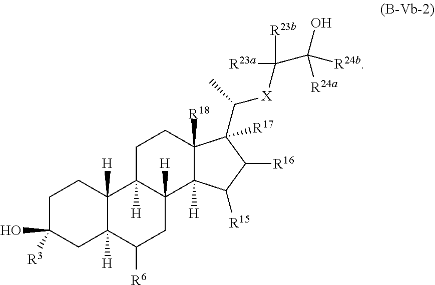

B-160. The compound according to any one of embodiments B-107 to B-110, B-127, and B-144 to B-152, wherein each of R^{2a} and R^{2b} is independently hydrogen.

B-161. The compound according to any one of embodiments B-107 to B-111, B-127 to B-128, and B-144 to B-152, wherein each of R^{11a} and R^{11b} is independently hydrogen.

B-162. The compound according to any one of embodiments B-107 to B-159 wherein R³ is unsubstituted C₁₋₆alkyl.

B-163. The compound according to embodiment B-162, wherein R³ is methyl, ethyl or propyl.

B-164. The compound according to embodiment B-163, wherein R³ is ethyl.

B-165. The compound according to any one of embodiments B-107 to B-159, wherein R⁶ is hydrogen.

B-166. The compound according to any one of embodiments B-107 to B-159, wherein R¹⁵ is hydrogen, substituted or unsubstituted C₁₋₆alkyl or substituted or unsubstituted C₃₋₆carbocyclyl.

B-167. The compound according to embodiment B-167, wherein R¹⁵ is hydrogen, unsubstituted C₁₋₆alkyl or unsubstituted C₃₋₆carbocyclyl.

B-168. The compound according to embodiment B-167, wherein R¹⁵ is hydrogen, —CH₃ or cyclopropyl.

B-169. The compound according to embodiment B-168, wherein R¹⁵ is hydrogen.

B-170. The compound according to embodiment B-168, wherein R¹⁵ is —CH₃.

B-171. The compound according to embodiment B-168, wherein R¹⁵ is cyclopropyl.

B-172. The compound according to any one of embodiments B-107 to B-159, wherein R¹⁶ is hydrogen, substituted or unsubstituted C₁₋₆alkyl or substituted or unsubstituted C₃₋₆carbocyclyl.

B-173. The compound according to embodiment B-172, wherein R¹⁶ is hydrogen or unsubstituted C₁₋₆alkyl.

B-174. The compound according to embodiment B-173, wherein R¹⁶ is hydrogen.

B-175. The compound according to embodiment B-173 wherein R¹⁶ is —CH₃.

B-176. The compound according to any one of embodiments B-107 to B-159, wherein R¹⁵ and R¹⁶ are taken together to form a substituted or unsubstituted C₃₋₆carbocyclyl.

B-177. The compound according to embodiment B-176, wherein R¹⁵ and R¹⁶ are taken together to form a cyclopropyl.

B-178. The compound according to embodiments B-107 to B-113, B-117 to B-120, B-124 to B-130, B-134 to B-137, B-141 to B-145, B-149, and B-153 to B-159, wherein R¹⁷ is hydrogen.

B-179. The compound according to embodiments B-107 to B-113, B-117 to B-120, B-124 to B-130, B-134 to B-140, B-141 to B-145, B-149, or B-153 to B-159, wherein R¹⁶ and R¹⁷ are taken together to form a substituted or unsubstituted C₃₋₆carbocyclyl.

B-180. The compound according to embodiment B-179, wherein R¹⁶ and R¹⁷ are taken together to form a cyclopropyl.

B-181. The compound according to any one of embodiments B-107 to B-159, wherein R¹⁸ is hydrogen, unsubstituted C₁₋₆alkyl or C₁₋₆alkyl substituted with OR^{A18}, wherein R^{A18} is substituted or unsubstituted C₁₋₆alkyl.

B-182. The compound according to any one of embodiments B-107 to B-159, wherein R¹⁸ is hydrogen, —CH₃, CH₂OCH₃, —OCH₃, —CH₂CH₃, or —CH(CH₃)₂.

B-183. The compound according to any one of embodiments B-107 to B-159, wherein R¹⁸ is —CH₃.

B-184. The compound according to any one of embodiments B-107, B-110 to B-112, B-127 to B-129, B-144, and B-153, wherein R¹⁹ is hydrogen, unsubstituted C₁₋₆alkyl or C₁₋₆alkyl substituted with OR^{A19}, wherein R^{A19} is substituted or unsubstituted C₁₋₆alkyl.

B-185. The compound according to embodiment B-184, wherein R¹⁹ is hydrogen, —CH₃, —CH₂OCH₃, —OCH₃, —CH₂CH₃, or —CH(CH₃)₂.

B-186. The compound according to embodiment B-185, wherein R¹⁹ is hydrogen, —CH₃, —CH₂CH₃.

B-187. The compound according to any one of embodiments B-107 to B-159, wherein X is —(C(R^{X})₂)ₙ—, wherein R^{X} is hydrogen or fluorine, and n is 1 or 2.

B-188. The compound according to embodiment B-187, wherein X is —CH₂— or —(CH₂)₂—.

B-189. The compound according to embodiment B-188, wherein X is —CH₂—.

B-190. The compound according to embodiment B-188, wherein X is —(CH₂)₂—.

B-191. The compound according to any one of embodiments B-107 to B-159, wherein each of R^{23a} and R^{23b} is independently hydrogen.

B-192. The compound according to any one of embodiments B-107 to B-159, wherein each of R^{23a} and R^{23b} is independently fluorine.

B-193. The compound according to any one of embodiments B-107 to B-159, wherein R^{24a} is selected from substituted or unsubstituted C₁₋₆alkyl.

B-194. The compound according to embodiment B-193, wherein R^{24a} is methyl, isopropyl or —CF₃.

B-195. The compound according to embodiment B-194, wherein R^{24a} is methyl.

B-196. The compound according to embodiment B-194, wherein R^{24a} is isopropyl.

B-197. The compound according to embodiment B-194, wherein R^{24a} is —CF₃.

B-198. The compound according to any one of embodiments B-107 to B-159, wherein R^{24b} is hydrogen or unsubstituted C₁₋₆alkyl.

B-199. The compound according to embodiment B-198, wherein R^{24b} is hydrogen or methyl.

B-200. The compound of embodiment B-107, wherein the compound of formula (I) is selected from the group consisting of Compounds B-1-B-24.

B-201. The compound according to embodiment B-107, wherein the compound of formula (B-I) is selected from the group consisting of Compounds B-1, B-3, B-4, B-10, B-11, B-12, B-14, B-15, B-18, B-20 and B-23.

B-202. The compound according to embodiment B-107, wherein the compound of formula (B-I) is selected from the group consisting of Compounds B-1, B-2, B-3, B-4, B-5, B-6, B-13, B-14, B-22, B-23, and B-24.

B-203. A pharmaceutical composition comprising a compound according to any one of embodiments B-107 to B-202, and a pharmaceutically acceptable carrier.

B-204. A method for treating a CNS-related condition in a subject, comprising administering to the subject an effective amount of a compound according to any one of embodiments B-107 to B-202, or a pharmaceutical composition according to embodiment B-203.

B-205. The method according to embodiment B-204, wherein the CNS-related condition is selected from the group consisting of an adjustment disorder, anxiety disorder (including obsessive-compulsive disorder, post-traumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease) and tinnitus.

B-206. A method of inducing sedation or anesthesia in a subject, comprising administering to the subject an effective amount of a compound according to any one of embodiments B-107 to B-202, or a pharmaceutical composition according to embodiment B-203.

B-207. A compound according to any one of embodiments B-107 to B-202, or a pharmaceutical composition according to embodiment B-203, for use in treating a CNS-related condition in a subject.

B-208. The compound or pharmaceutical composition for use according to embodiment B-207, wherein the CNS-related condition is selected from the group consisting of an adjustment disorder, anxiety disorder (including obsessive-compulsive disorder, post-traumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease) and tinnitus.

B-209. A compound according to any one of embodiments B-107 to B-202, or a pharmaceutical composition according to embodiment B-203, for use inducing sedation or anesthesia in a subject.

B-210. Use of a compound according to any one of embodiments B-107 to B-202, or a pharmaceutical composition according to embodiment B-203, for the manufacture of a medicament for treating a CNS-related condition in a subject.

C-1. A compound of Formula (C-I):

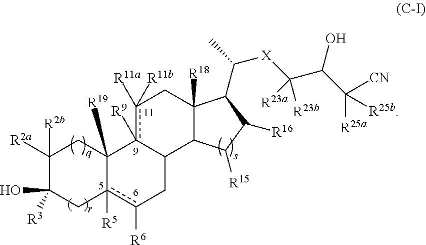

or a pharmaceutically acceptable salt thereof;
wherein:
   each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein R^{A2} is hydrogen or substituted or unsubstituted C₁₋₆alkyl;
   R³ is substituted or unsubstituted C₁₋₆alkyl, substituted or unsubstituted C₂₋₆alkenyl, or substituted or unsubstituted C₂₋₆alkynyl, or substituted or unsubstituted C₃₋₁₀carbocyclyl;
   R⁵ is absent, hydrogen, halogen, or substituted or unsubstituted C₁₋₆alkyl;
   R⁶ is hydrogen, halogen, or substituted or unsubstituted C₁₋₆alkyl;
   R⁹ is absent or hydrogen; or
   R⁹ and R^{11a} are taken together with the carbon atoms to which they are attached to form a substituted or unsubstituted C₃₋₆carbocyclyl ring;
   R^{11a} is hydrogen, halogen, substituted or unsubstituted C₁₋₆alkyl, or —OR^{A11}, wherein R^{A11} is hydrogen or substituted or unsubstituted C₁₋₆alkyl;
   R^{11b} is absent or hydrogen; or
   R^{11a} and R^{11b} are joined to form an oxo (═O) group;
   each instance of R¹⁵ is hydrogen, halogen, —OH, —NH₂, substituted or unsubstituted C₁₋₆alkyl, substituted or unsubstituted C₂₋₆alkenyl, substituted or unsubstituted C₂₋₆alkynyl, substituted or unsubstituted C₁₋₆alkoxy, substituted or unsubstituted C₃₋₆carbocyclyl, or substituted or unsubstituted 3- to 10-membered heterocyclyl;
   R¹⁶ is hydrogen, halogen, —OH, —NH₂, substituted or unsubstituted C₁₋₆alkyl, substituted or unsubstituted C₂₋₆alkenyl, substituted or unsubstituted C₂₋₆alkynyl, substituted or unsubstituted C₁₋₆alkoxy, substituted or unsubstituted C₃₋₆carbocyclyl, or substituted or unsubstituted 3- to 10-membered heterocyclyl; or
   R¹⁵ and R¹⁶ are taken together with the carbon atoms to which they are attached, to form a substituted or unsubstituted C₃₋₆carbocyclyl;
   R¹⁸ is hydrogen, substituted or unsubstituted C₁₋₆alkyl, or OR^{A18}, wherein each instance of R^{A18} is independently substituted or unsubstituted C₁₋₆alkyl;
   R¹⁹ is hydrogen, substituted or unsubstituted C₁₋₆alkyl, or OR^{A19}; wherein each instance of R^{A19} is independently substituted or unsubstituted C₁₋₆alkyl;
   X is —(C(R^{X})₂)ₙ— or —O—, wherein R^{X} is hydrogen or fluorine, or one R^{X} group and R^{23b} are joined to form a double bond;
   n is 1, 2 or 3;
   each of R^{23a} and R^{23b} is independently hydrogen or fluorine;
   R^{25a} is hydrogen, substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, or substituted or unsubstituted 5- to 10-membered heteroaryl;
   R^{25b} is hydrogen, substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, or substituted or unsubstituted 5- to 10-membered heteroaryl;
   or R^{25a} and R^{25b}, taken together with the carbon atom to which they are attached, form a substituted or unsubstituted C₃₋₆carbocyclyl or substituted or unsubstituted 3- to 10-membered heterocyclyl;
   q is 0, 1 or 2;
   r is 1 or 2;
   s is 1 or 2; and
   each [symbol] independently represents a single or double bond;
   provided if the bond between C5 and C6 is a double bond, R⁵ is absent and if the bond between C9 and C11 is a double bond, R⁹ and R^{11b} are absent.

C-2. The compound or pharmaceutically acceptable salt thereof according to embodiment C-1, wherein the compound of Formula (C-I) is a compound of Formula (C-II):

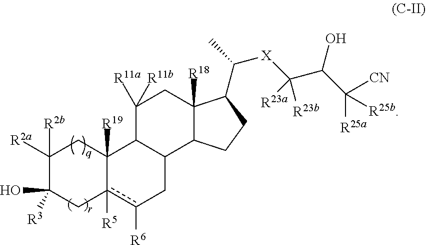

C-3. The compound or pharmaceutically acceptable salt thereof according to embodiment C-2, wherein the compound of Formula (C-II) is a compound of Formula (C-II-A):

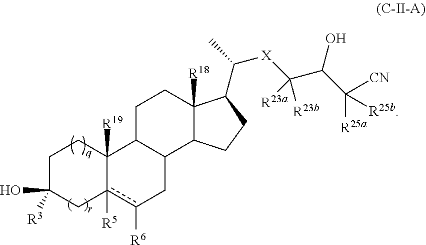

C-4. The compound or pharmaceutically acceptable salt thereof according to embodiment C-3, wherein the compound of Formula (C-II-A) is a compound of Formula (C-II-A1):

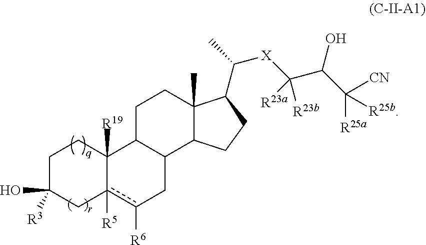

C-5. The compound or pharmaceutically acceptable salt thereof according to embodiment C-4, wherein the compound of Formula (C-II-A1) is a compound of Formula (C-II-A1-i):

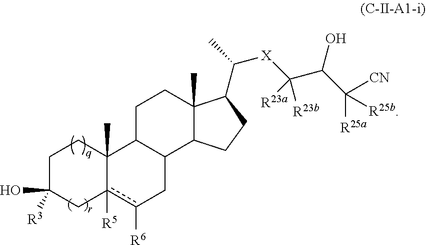

C-6. The compound or pharmaceutically acceptable salt thereof according to embodiment C-5, wherein the compound of Formula (C-II-A1-i) is a compound of Formula (C-II-A1-ia):

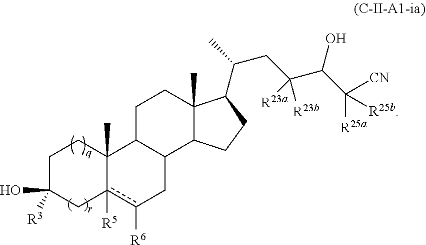

C-7. The compound or pharmaceutically acceptable salt thereof according to embodiment C-6, wherein the compound of Formula (C-II-A1-ia) is a compound of Formula (C-II-A1-ia-1):

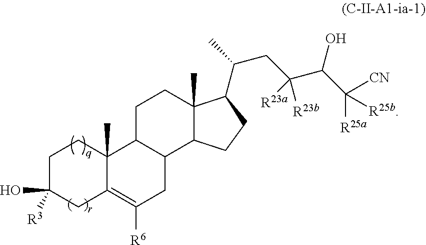

C-8. The compound or pharmaceutically acceptable salt thereof according to embodiment C-7, wherein the compound of Formula (C-II-A1-ia-1) is a compound of Formula (C-II-A1-ia-1a) or Formula (C-II-A1-ia-1b):

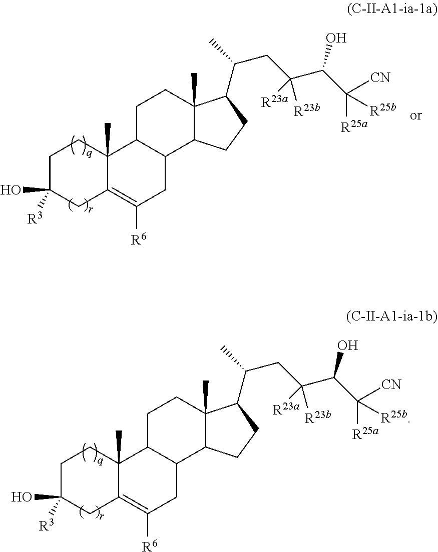

C-9. The compound or pharmaceutically acceptable salt thereof according to embodiment C-6, wherein the compound of Formula (C-II-A1-ia) is a compound of Formula (C-II-A1-ia-2):

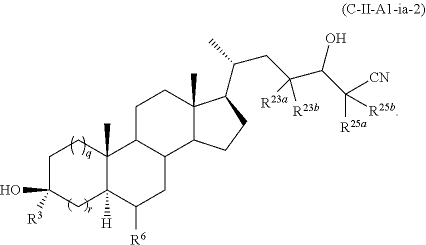

C-10. The compound or pharmaceutically acceptable salt thereof according to embodiment C-9, wherein the compound of Formula (C-II-A1-ia-2) is a compound of Formula (C-II-A1-ia-2a) or Formula (C-II-A1-ia-2b):

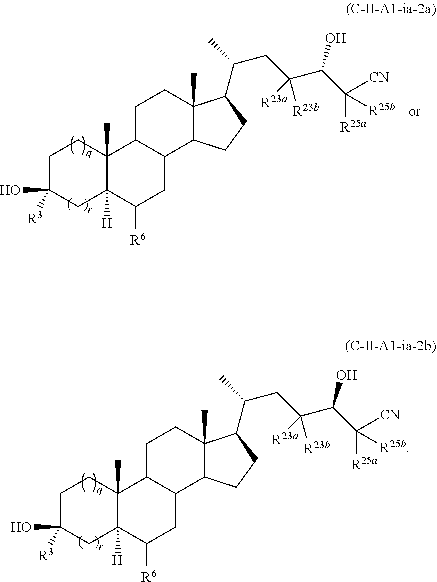

C-11. The compound or pharmaceutically acceptable salt thereof according to embodiment C-4, wherein the compound of Formula (C-II-A1) is a compound of Formula (C-II-A1-ii):

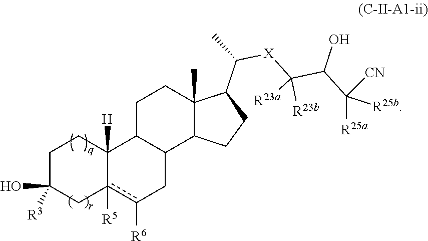

C-12. The compound or pharmaceutically acceptable salt thereof according to embodiment C-11, wherein the compound of Formula (C-II-A1-ii) is a compound of Formula (C-II-A1-iia):

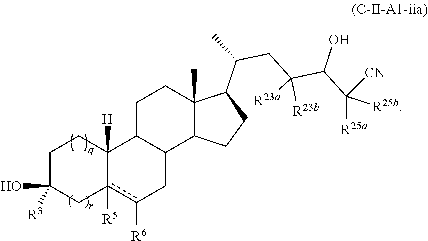

C-13. The compound or pharmaceutically acceptable salt thereof according to embodiment C-12, wherein the compound of Formula (C-II-A1-iia) is a compound of Formula (C-II-A1-iia-1):

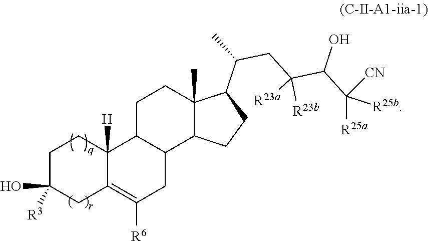

C-14. The compound or pharmaceutically acceptable salt thereof according to embodiment C-13, wherein the compound of Formula (C-II-A1-iia-1) is a compound of Formula (C-II-A1-iia-1a) or Formula (C-II-A1-iia-1b):

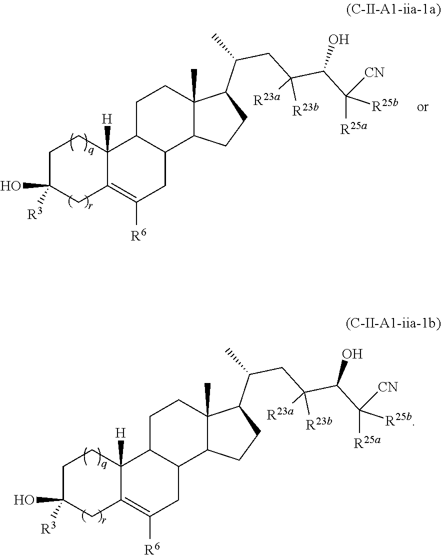

C-15. The compound or pharmaceutically acceptable salt thereof according to embodiment C-12, wherein the compound of Formula (C-II-A1-iia) is a compound of Formula (C-II-A1-iia-2):

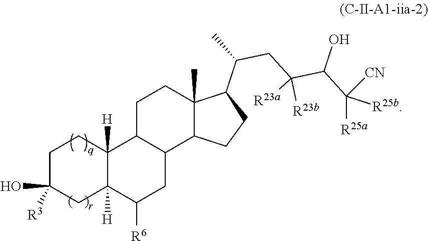

C-16. The compound or pharmaceutically acceptable salt thereof according to embodiment C-15, wherein the compound of Formula (C-II-A1-iia-2) is a compound of Formula (C-II-A1-iia-2a) or Formula (C-II-A1-iia-2b):

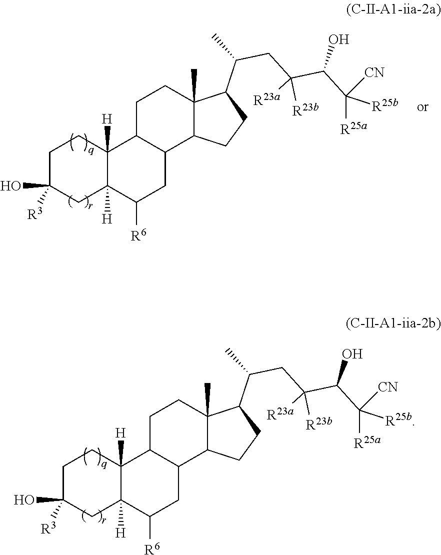

C-17. The compound or pharmaceutically acceptable salt thereof according to embodiment C-1, wherein the compound of Formula (C-I) is a compound of Formula (C-III):

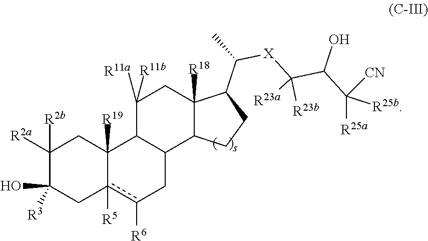

C-18. The compound or pharmaceutically acceptable salt thereof according to embodiment C-17, wherein the compound of Formula (C-III) is a compound of Formula (C-III-A):

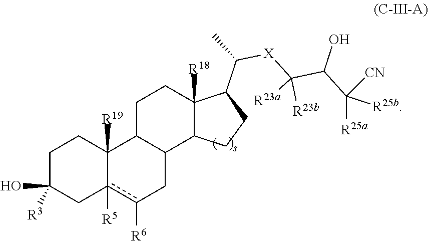

C-19. The compound or pharmaceutically acceptable salt thereof according to embodiment C-18, wherein the compound of Formula (C-III-A) is a compound of Formula (C-III-A1):

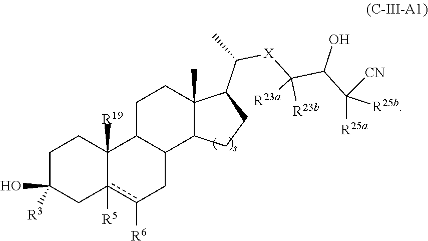

C-20. The compound or pharmaceutically acceptable salt thereof according to embodiment C-19, wherein the compound of Formula (C-III-A1) is a compound of Formula (C-III-A1-i):

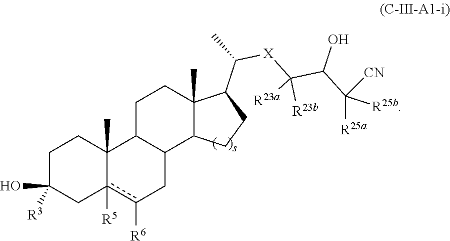

C-21. The compound or pharmaceutically acceptable salt thereof according to embodiment C-20, wherein the compound of Formula (C-III-A1-i) is a compound of Formula (C-III-A1-ia):

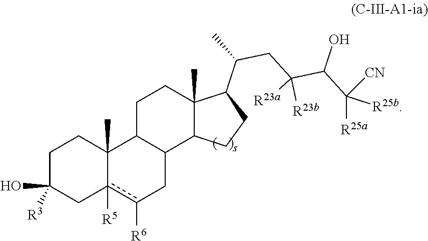

C-22. The compound or pharmaceutically acceptable salt thereof according to embodiment C-21, wherein the compound of Formula (C-III-A1-ia) is a compound of Formula (C-III-A1-ia-1):

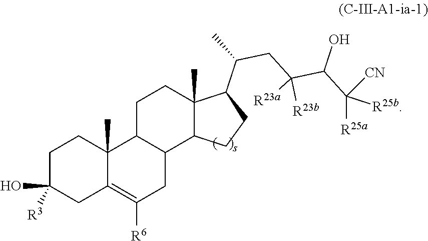

C-23. The compound or pharmaceutically acceptable salt thereof according to embodiment C-22, wherein the compound of Formula (C-III-A1-ia-1) is a compound of Formula (C-III-A1-ia-1a) or Formula (C-III-A1-ia-1b):

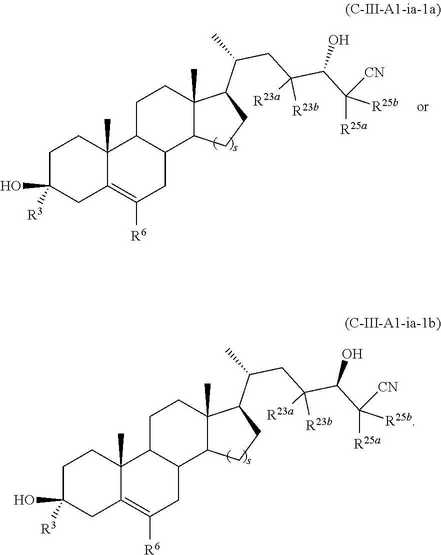

C-24. The compound or pharmaceutically acceptable salt thereof according to embodiment C-21, wherein the compound of Formula (C-III-A1-ia) is a compound of Formula (C-III-A1-ia-2):

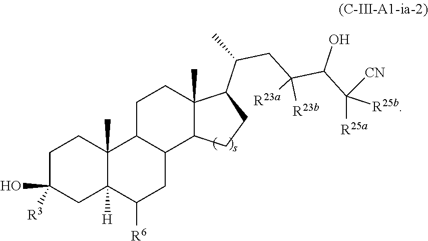

C-25. The compound or pharmaceutically acceptable salt thereof according to embodiment C-24, wherein the compound of Formula (C-III-A1-ia-2) is a compound of Formula (C-III-A1-ia-2a) or Formula (C-III-A1-ia-2b):

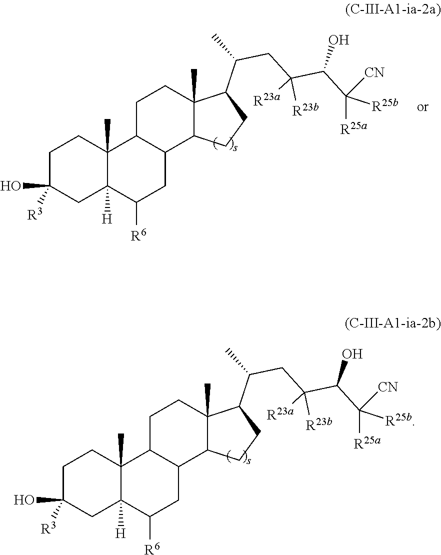

C-26. The compound or pharmaceutically acceptable salt thereof according to embodiment C-19, wherein the compound of Formula (C-III-A1) is a compound of Formula (C-III-A1-ii):

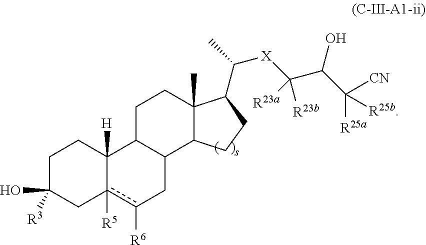

C-27. The compound or pharmaceutically acceptable salt thereof according to embodiment C-26, wherein the compound of Formula (C-III-A1-ii) is a compound of Formula (C-III-A1-iia):

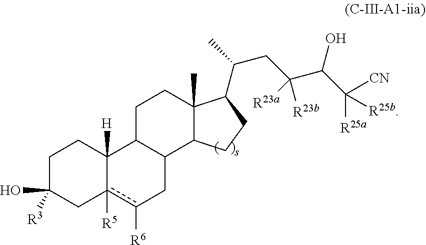

C-28. The compound or pharmaceutically acceptable salt thereof according to embodiment C-27, wherein the compound of Formula (C-III-A1-iia) is a compound of Formula (C-III-A1-iia-1):

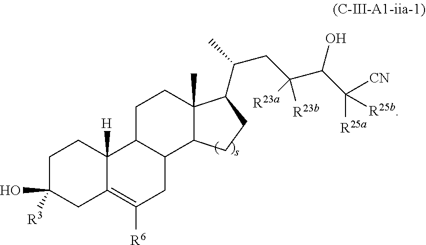

C-29. The compound or pharmaceutically acceptable salt thereof according to embodiment C-28, wherein the compound of Formula (C-III-A1-iia-1) is a compound of Formula (C-III-A1-iia-1a) or Formula (C-III-A1-iia-1b):

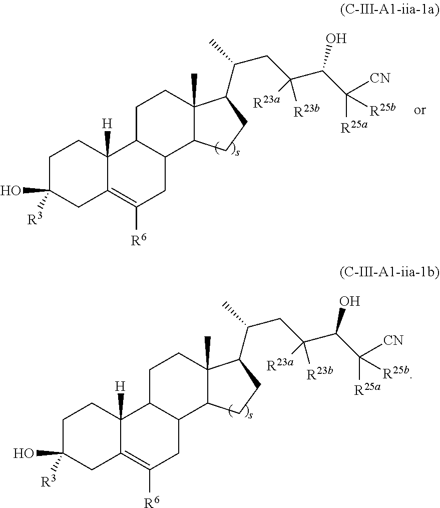

C-30. The compound or pharmaceutically acceptable salt thereof according to embodiment C-27, wherein the compound of Formula (C-III-A1-iia) is a compound of Formula (C-III-A1-iia-2):

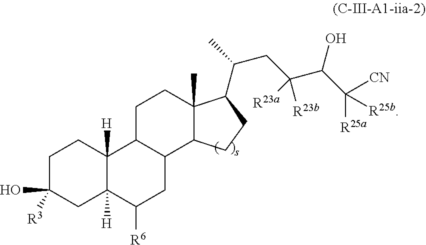

C-31. The compound or pharmaceutically acceptable salt thereof according to embodiment C-30, wherein the compound of Formula (C-III-A1-iia-2) is a compound of Formula (C-III-A1-iia-2a) or Formula (C-III-A1-iia-2b):

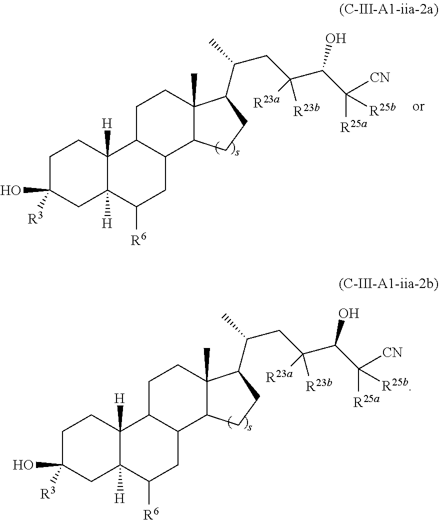

C-32. The compound or pharmaceutically acceptable salt thereof according to embodiment C-1, wherein the compound of Formula (C-I) is a compound of Formula (C-IV):

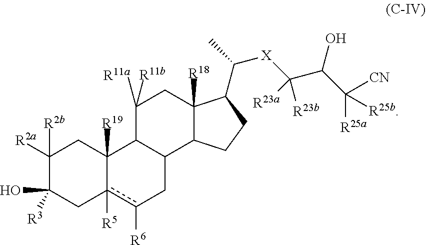

C-33. The compound or pharmaceutically acceptable salt thereof according to embodiment C-32, wherein the compound of Formula (C-IV) is a compound of Formula (C-IV-A):

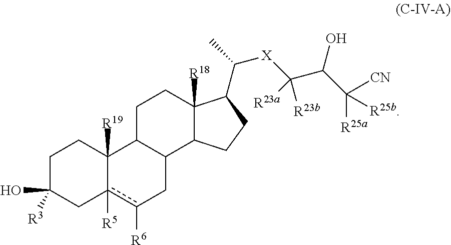

C-34. The compound or pharmaceutically acceptable salt thereof according to embodiment C-33, wherein the compound of Formula (C-IV-A) is a compound of Formula (C-IV-A1):

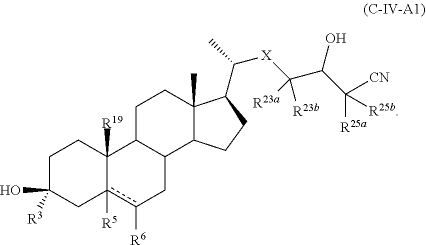

C-35. The compound or pharmaceutically acceptable salt thereof according to embodiment C-34, wherein the compound of Formula (C-IV-A1) is a compound of Formula (C-IV-A1-i):

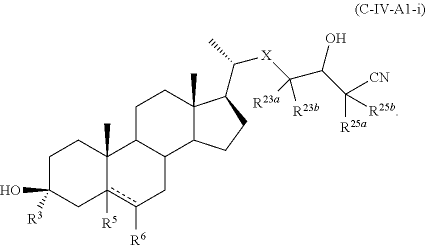

C-36. The compound or pharmaceutically acceptable salt thereof according to embodiment C-35, wherein the compound of Formula (C-IV-A1-i) is a compound of Formula (C-IV-A1-ia):

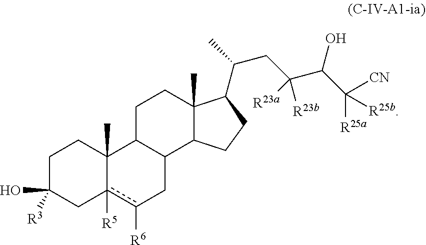

C-37. The compound or pharmaceutically acceptable salt thereof according to embodiment C-36, wherein the compound of Formula (C-IV-A1-ia) is a compound of Formula (C-IV-A1-ia-1):

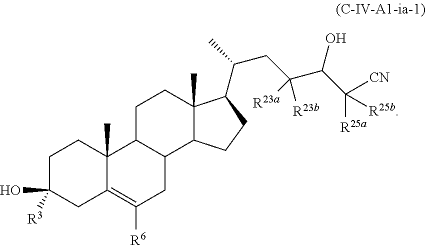

C-38. The compound or pharmaceutically acceptable salt thereof according to embodiment C-37, wherein the compound of Formula (C-IV-A1-ia-1) is a compound of Formula (C-IV-A1-ia-la) or Formula (C-IV-A1-ia-1b):

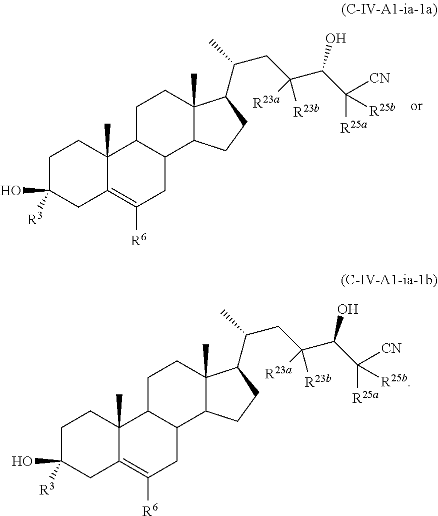

C-39. The compound or pharmaceutically acceptable salt thereof according to embodiment C-36, wherein the compound of Formula (C-IV-A1-ia) is a compound of Formula (C-IV-A1-ia-2):

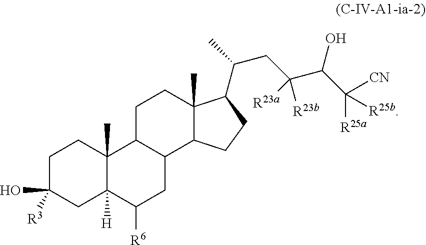

C-40. The compound or pharmaceutically acceptable salt thereof according to embodiment C-39, wherein the compound of Formula (C-IV-A1-ia-2) is a compound of Formula (C-IV-A1-ia-2a) or Formula (C-IV-A1-ia-2b):

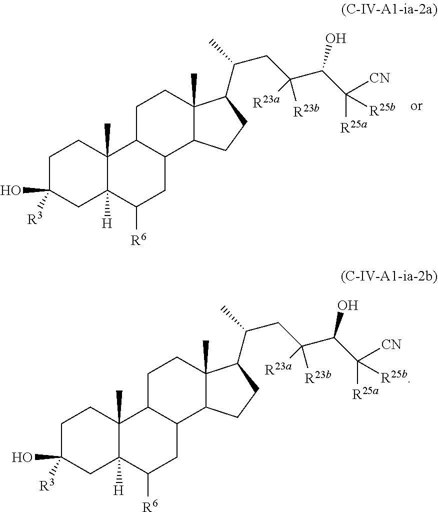

C-41. The compound or pharmaceutically acceptable salt thereof according to embodiment C-34, wherein the compound of Formula (C-IV-A1) is a compound of Formula (C-IV-A1-ii):

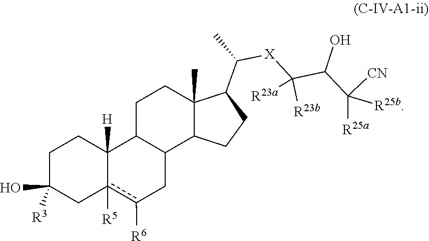

C-42. The compound or pharmaceutically acceptable salt thereof according to embodiment C-41, wherein the compound of Formula (C-IV-A1-ii) is a compound of Formula (C-IV-A1-iia):

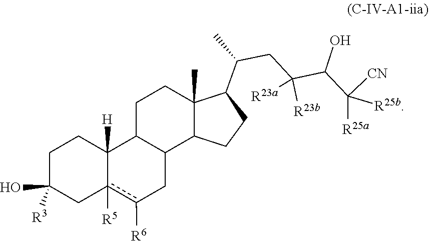

C-43. The compound or pharmaceutically acceptable salt thereof according to embodiment C-42, wherein the compound of Formula (C-IV-A1-iia) is a compound of Formula (C-IV-A1-iia-1):

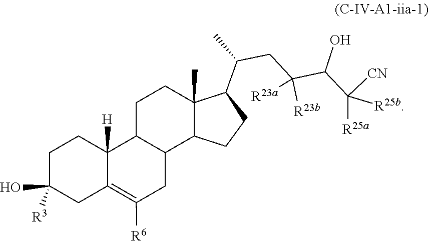

C-44. The compound or pharmaceutically acceptable salt thereof according to embodiment C-43, wherein the compound of Formula (C-IV-A1-iia-1) is a compound of Formula (C-IV-A1-iia-1a) or Formula (C-IV-A1-iia-1b):

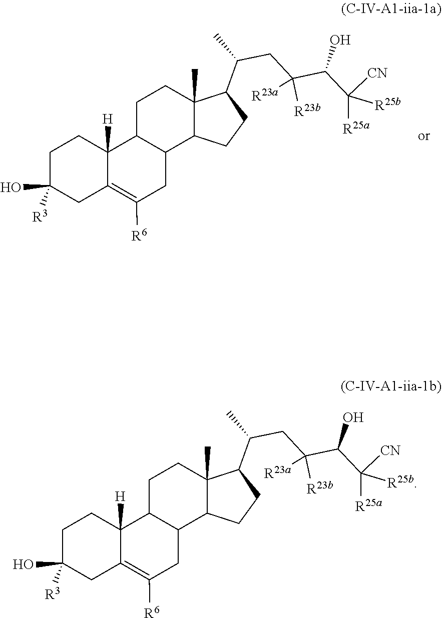

C-45. The compound or pharmaceutically acceptable salt thereof according to embodiment C-42, wherein the compound of Formula (C-IV-A1-iia) is a compound of Formula (C-IV-A1-iia-2):

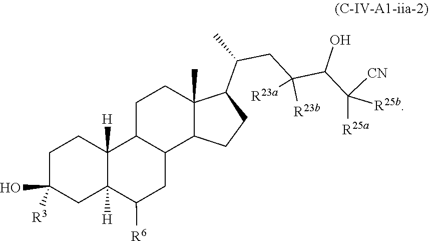

C-46. The compound or pharmaceutically acceptable salt thereof according to embodiment C-45, wherein the compound of Formula (C-IV-A1-iia-2) is a compound of Formula (C-IV-A1-iia-2a) or Formula (C-IV-A1-iia-2b):

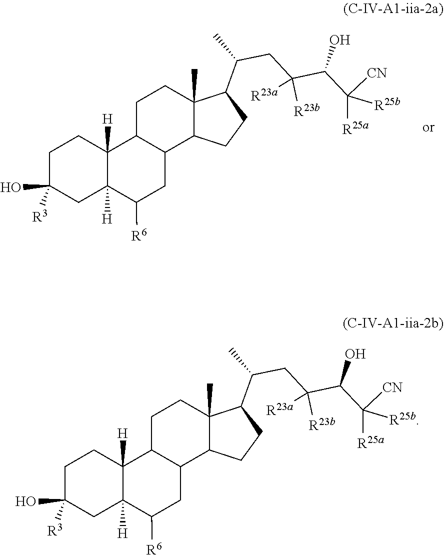

C-47. The compound or pharmaceutically acceptable salt according to embodiment C-1, wherein the compound of Formula (C-I) is a compound of Formula (C-V):

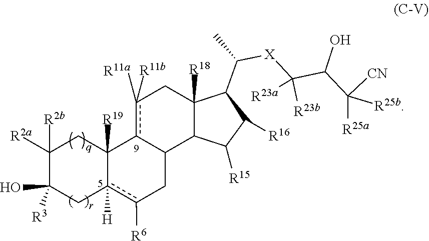

C-48. The compound or pharmaceutically acceptable salt according to embodiment C-47, wherein the compound of Formula (C-V) is a compound of Formula (C-V-A) or Formula (C-V-B):

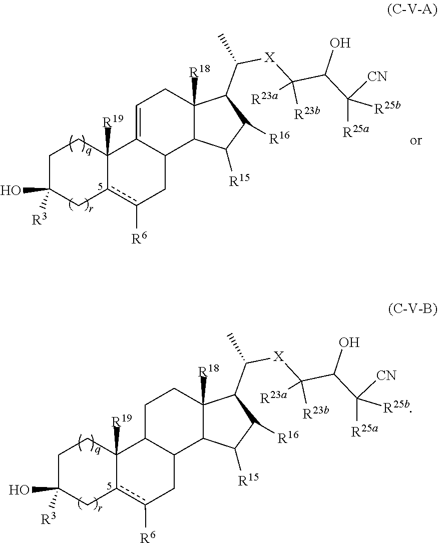

C-49. The compound or pharmaceutically acceptable salt thereof according to embodiment C-48, wherein the compound of Formula (C-V-A) is a compound of Formula (C-V-A1):

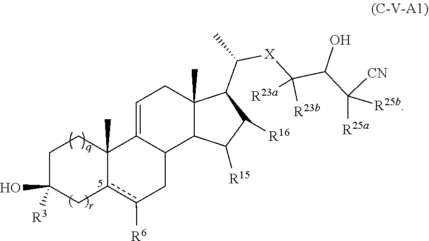

C-50. The compound or pharmaceutically acceptable salt thereof according to embodiment C-49, wherein the compound of Formula (C-V-A1) is a compound of Formula (C-V-A1-i):

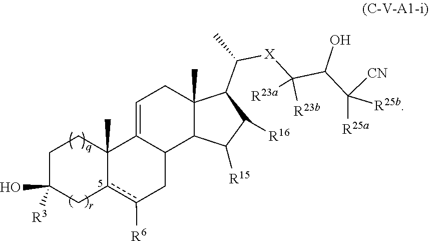

C-51. The compound or pharmaceutically acceptable salt thereof according to embodiment C-50, wherein the compound of Formula (C-V-A1-i) is a compound of Formula (C-V-A1-ia):

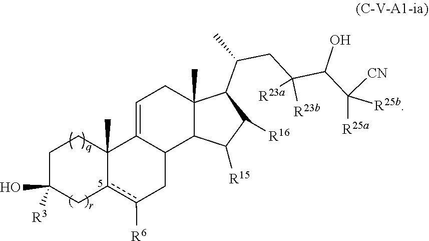

C-52. The compound or pharmaceutically acceptable salt thereof according to embodiment C-51, wherein the compound of Formula (C-V-A1-ia) is a compound of Formula (C-V-A1-ia-1):

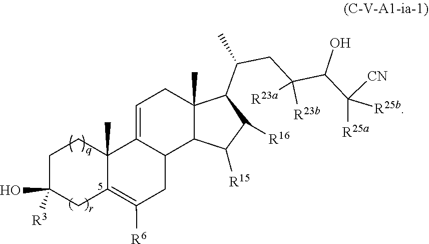

C-53. The compound or pharmaceutically acceptable salt thereof according to embodiment C-52, wherein the compound of Formula (C-V-A1-ia-1) is a compound of Formula (C-V-A1-ia-la) or Formula (C-V-A1-ia-1b):

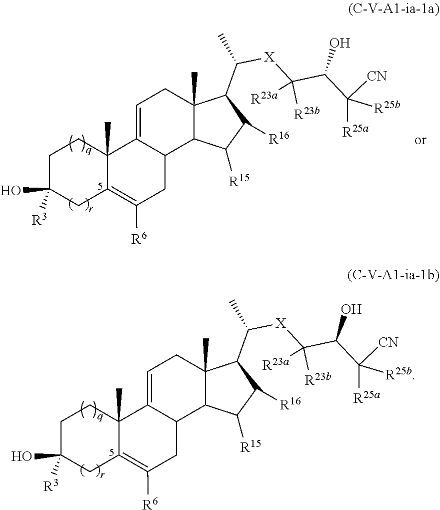

C-54. The compound or pharmaceutically acceptable salt thereof according to embodiment C-51, wherein the compound of Formula (C-V-A1-ia) is a compound of Formula (C-V-A1-ia-2):

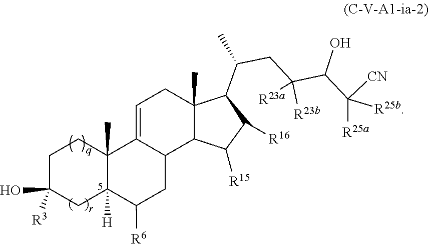

C-55. The compound or pharmaceutically acceptable salt thereof according to embodiment C-54, wherein the compound of Formula (C-V-A1-ia-2) is a compound of Formula (C-V-A1-ia-2a) or Formula (C-V-A1-ia-2b):

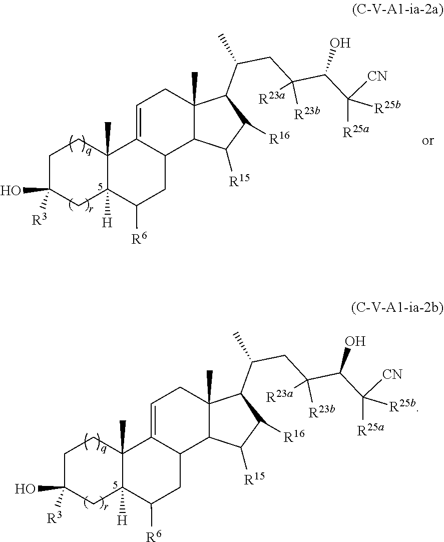

C-56. The compound or pharmaceutically acceptable salt thereof according to embodiment C-49, wherein the compound of Formula (C-V-A1) is a compound of Formula (C-V-A1-ii):

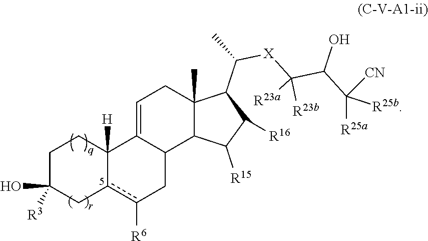

C-57. The compound or pharmaceutically acceptable salt thereof according to embodiment C-56, wherein the compound of Formula (C-V-A1-ii) is a compound of Formula (C-V-A1-iia):

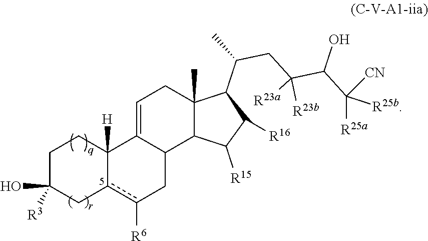

C-58. The compound or pharmaceutically acceptable salt thereof according to embodiment C-57, wherein the compound of Formula (C-V-A1-iia) is a compound of Formula (C-V-A1-iia-1):

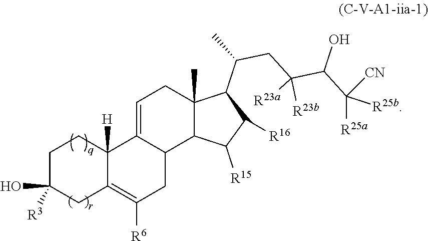

C-59. The compound or pharmaceutically acceptable salt thereof according to embodiment C-58, wherein the compound of Formula (C-V-A1-iia-1) is a compound of Formula (C-V-A1-iia-1a) or Formula (C-V-A1-iia-1b):

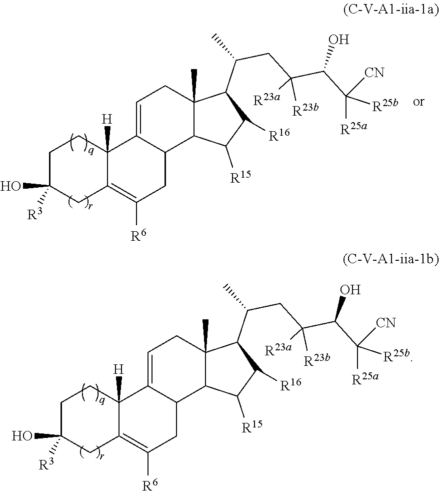

C-60. The compound or pharmaceutically acceptable salt thereof according to embodiment C-57, wherein the compound of Formula (C-V-A1-iia) is a compound of Formula (C-V-A1-iia-2):

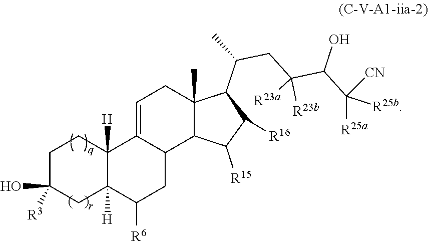

C-61. The compound or pharmaceutically acceptable salt thereof according to embodiment C-60, wherein the compound of Formula (C-V-A1-iia-2) is a compound of Formula (C-V-A1-iia-2a) or Formula (C-V-A1-iia-2b):

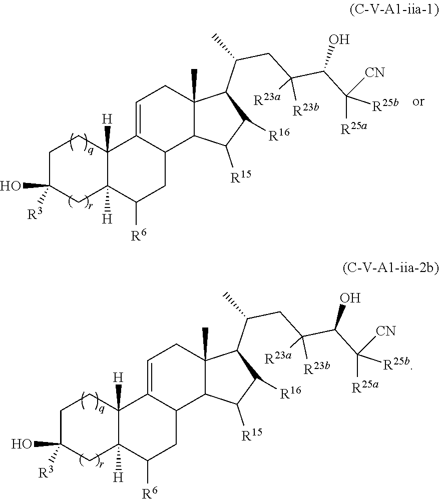

C-62. The compound or pharmaceutically acceptable salt thereof according to embodiment C-48, wherein the compound of Formula (C-V-B) is a compound of Formula (C-V-B1):

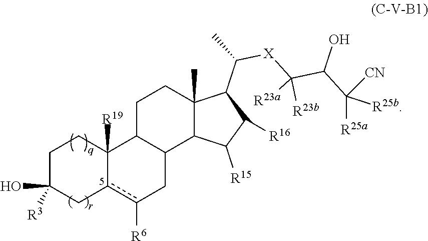

C-63. The compound or pharmaceutically acceptable salt thereof according to embodiment C-62, wherein the compound of Formula (C-V-B1) is a compound of Formula (C-V-B1-i):

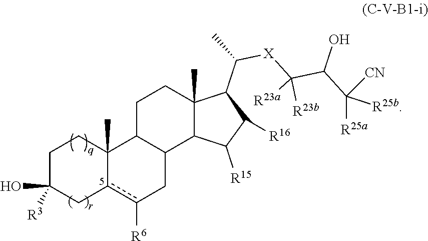

C-64. The compound or pharmaceutically acceptable salt thereof according to embodiment C-63, wherein the compound of Formula (C-V-B1-i) is a compound of Formula (C-V-B1-ia):

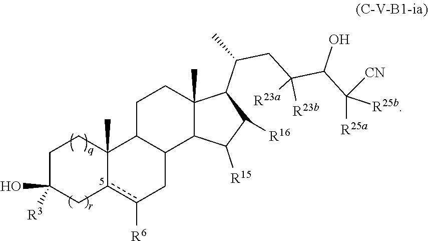

C-65. The compound or pharmaceutically acceptable salt thereof according to embodiment C-64, wherein the compound of Formula (C-V-B1-ia) is a compound of Formula (C-V-B1-ia-1):

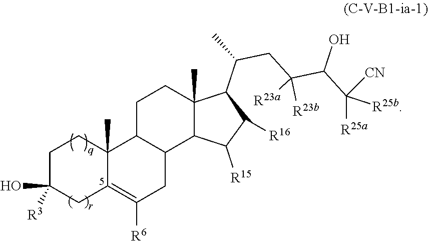

C-66. The compound or pharmaceutically acceptable salt thereof according to embodiment C-65, wherein the compound of Formula (C-V-B1-ia-1) is a compound of Formula (C-V-B1-ia-la) or Formula (C-V-B1-ia-1b):

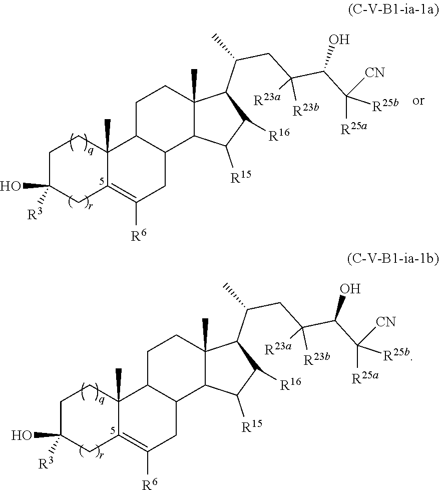

C-67. The compound or pharmaceutically acceptable salt thereof according to embodiment C-64, wherein the compound of Formula (C-V-B1-ia) is a compound of Formula (C-V-B1-ia-2):

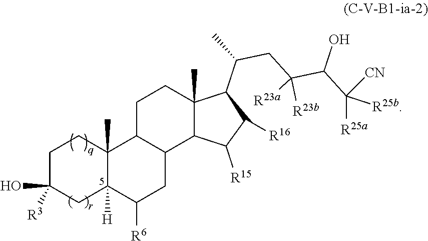

C-68. The compound or pharmaceutically acceptable salt thereof according to embodiment C-67, wherein the compound of Formula (C-V-B1-ia-2) is a compound of Formula (C-V-B1-ia-2a) or Formula (C-V-B1-ia-2b):

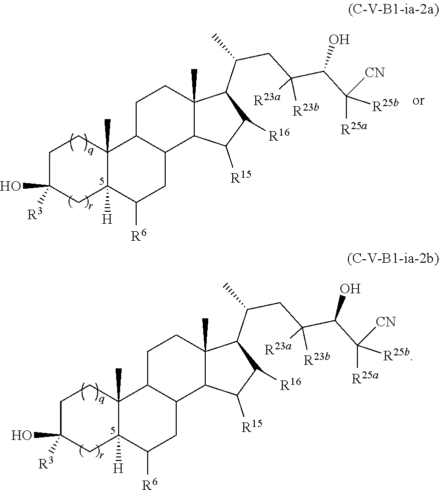

C-69. The compound or pharmaceutically acceptable salt thereof according to embodiment C-62, wherein the compound of Formula (C-V-B1) is a compound of Formula (C-V-B1-ii):

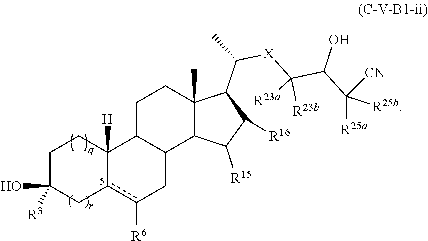

C-70. The compound or pharmaceutically acceptable salt thereof according to embodiment C-69, wherein the compound of Formula (C-V-B1-ii) is a compound of Formula (C-V-B1-iia):

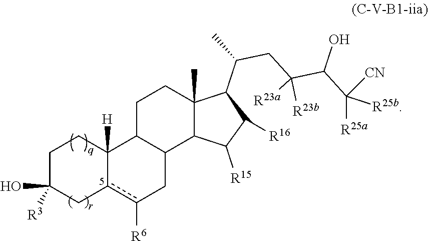

C-71. The compound or pharmaceutically acceptable salt thereof according to embodiment C-70, wherein the compound of Formula (C-V-B1-iia) is a compound of Formula (C-V-B1-iia-1):

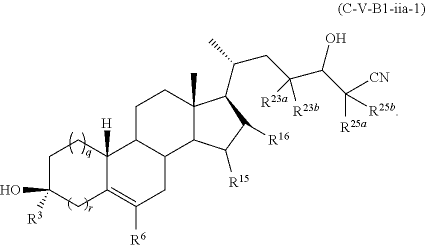

C-72. The compound or pharmaceutically acceptable salt thereof according to embodiment C-71, wherein the compound of Formula (C-V-B1-iia-1) is a compound of Formula (C-V-B1-iia-1a) or Formula (C-V-B1-iia-1b):

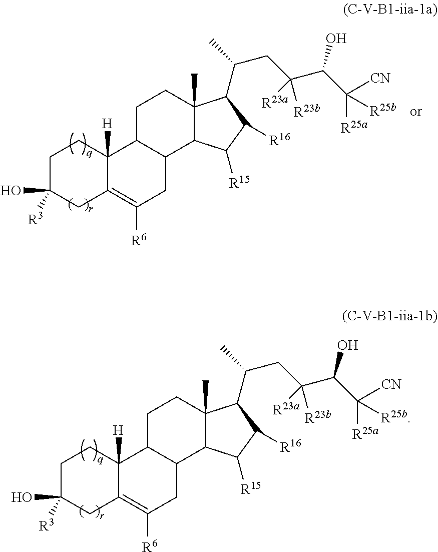

C-73. The compound or pharmaceutically acceptable salt thereof according to embodiment C-70, wherein the compound of Formula (C-V-B1-iia) is a compound of Formula (C-V-B1-iia-2):

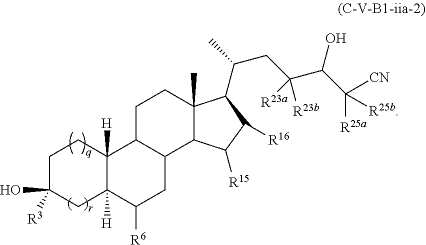

C-74. The compound or pharmaceutically acceptable salt thereof according to embodiment C-73, wherein the compound of Formula (C-V-B1-iia-2) is a compound of Formula (C-V-B1-iia-2a) or Formula (C-V-B1-iia-2b):

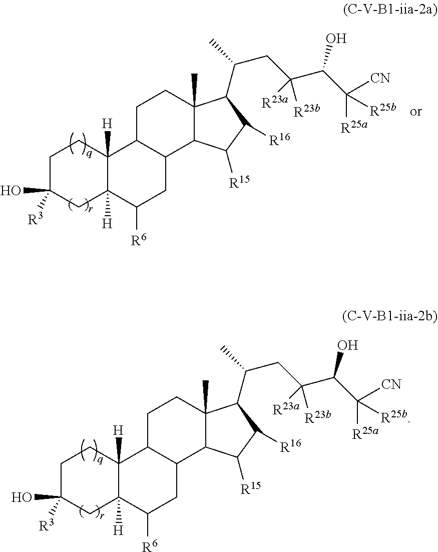

C-75. The compound or pharmaceutically acceptable salt according to embodiment C-1, wherein the compound of Formula (C-I) is a compound of Formula (C-VI):

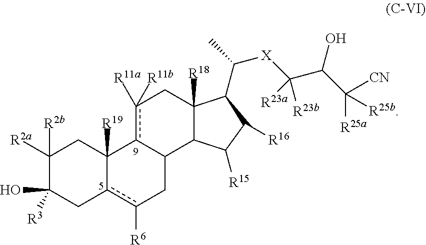

C-76. The compound or pharmaceutically acceptable salt according to embodiment C-75, wherein the compound of Formula (C-VI) is a compound of Formula (C-VI-A) or Formula (C-VI-B):

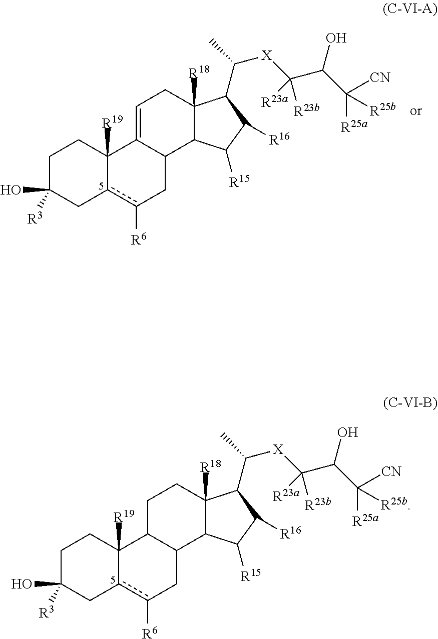

C-77. The compound or pharmaceutically acceptable salt thereof according to embodiment C-76, wherein the compound of Formula (C-VI-A) is a compound of Formula (C-VI-A1):

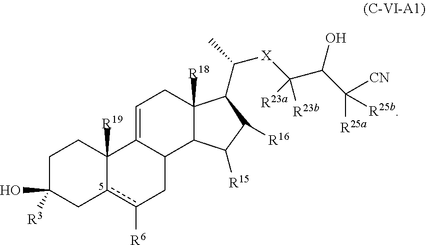

C-78. The compound or pharmaceutically acceptable salt thereof according to embodiment C-77, wherein the compound of Formula (C-VI-A1) is a compound of Formula (C-VI-A1-i):

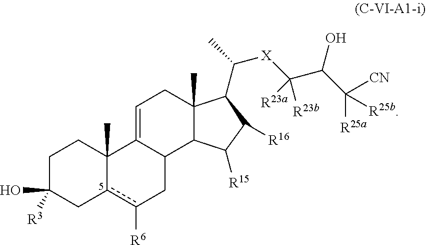

C-79. The compound or pharmaceutically acceptable salt thereof according to embodiment C-78, wherein the compound of Formula (C-VI-A1-i) is a compound of Formula (C-VI-A1-ia):

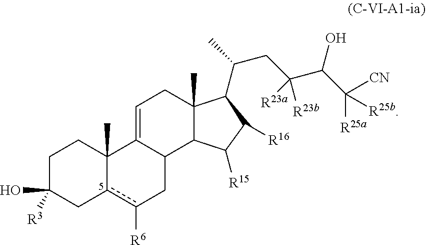

C-80. The compound or pharmaceutically acceptable salt thereof according to embodiment C-79, wherein the compound of Formula (C-VI-A1-ia) is a compound of Formula (C-VI-A1-ia-1):

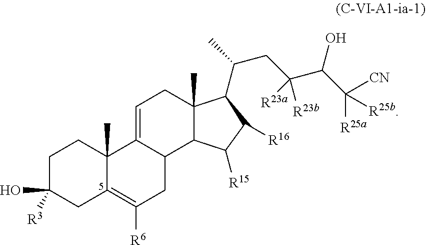

C-81. The compound or pharmaceutically acceptable salt thereof according to embodiment C-80, wherein the compound of Formula (C-VI-A1-ia-1) is a compound of Formula (C-VI-A1-ia-la) or Formula (C-VI-A1-ia-1b):

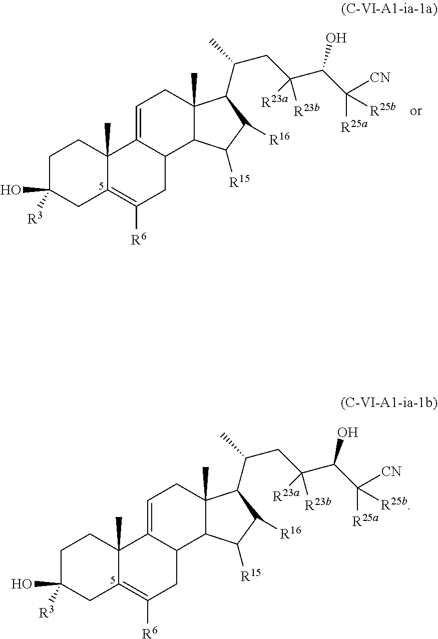

C-82. The compound or pharmaceutically acceptable salt thereof according to embodiment C-79, wherein the compound of Formula (C-VI-A1-ia) is a compound of Formula (C-VI-A1-ia-2):

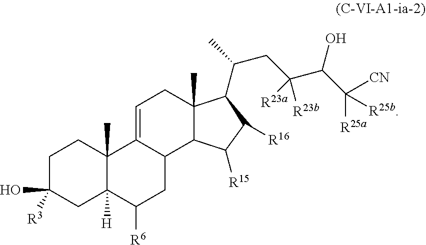

C-83. The compound or pharmaceutically acceptable salt thereof according to embodiment C-82, wherein the compound of Formula (C-VI-A1-ia-2) is a compound of Formula (C-VI-A1-ia-2a) or Formula (C-VI-A1-ia-2b):

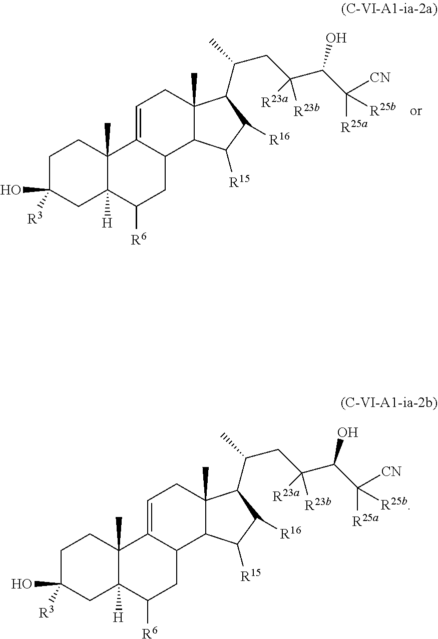

C-84. The compound or pharmaceutically acceptable salt thereof according to embodiment C-77, wherein the compound of Formula (C-VI-A1) is a compound of Formula (C-VI-A1-ii):

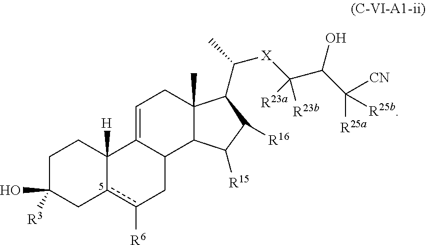

C-85. The compound or pharmaceutically acceptable salt thereof according to embodiment C-84, wherein the compound of Formula (C-VI-A1-ii) is a compound of Formula (C-VI-A1-iia):

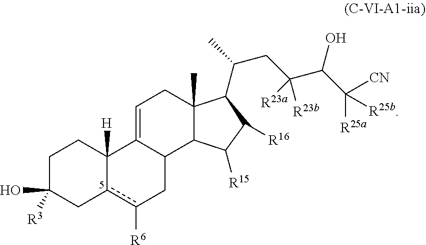

C-86. The compound or pharmaceutically acceptable salt thereof according to embodiment C-85, wherein the compound of Formula (C-VI-A1-iia) is a compound of Formula (C-VI-A1-iia-1):

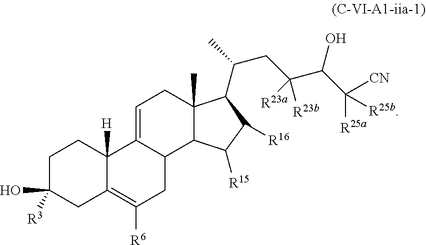

C-87. The compound or pharmaceutically acceptable salt thereof according to embodiment C-86, wherein the compound of Formula (C-VI-A1-iia-1) is a compound of Formula (C-VI-A1-iia-1a) or Formula (C-VI-A1-iia-1b):

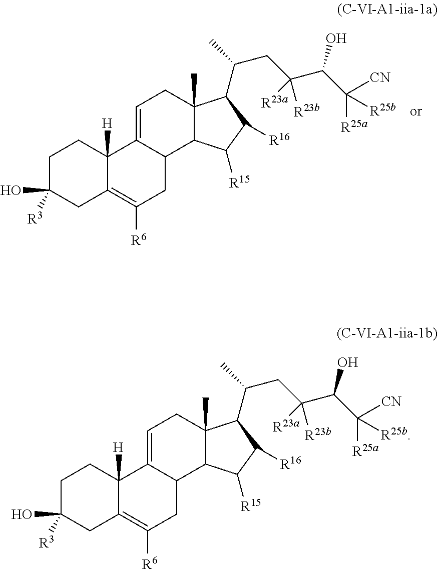

C-88. The compound or pharmaceutically acceptable salt thereof according to embodiment C-85, wherein the compound of Formula (C-VI-A1-iia) is a compound of Formula (C-VI-A1-iia-2):

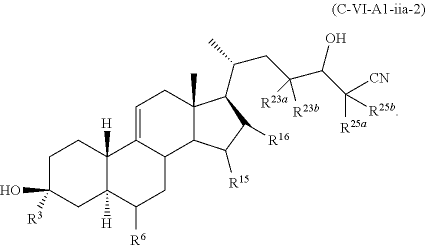

C-89. The compound or pharmaceutically acceptable salt thereof according to embodiment C-88, wherein the compound of Formula (C-VI-A1-iia-2) is a compound of Formula (C-VI-A1-iia-2a) or Formula (C-VI-A1-iia-2b):

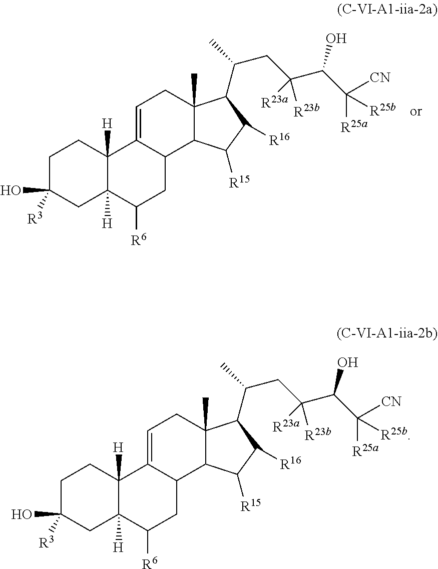

C-90. The compound or pharmaceutically acceptable salt thereof according to embodiment C-76, wherein the compound of Formula (C-VI-B) is a compound of Formula (C-VI-B1):

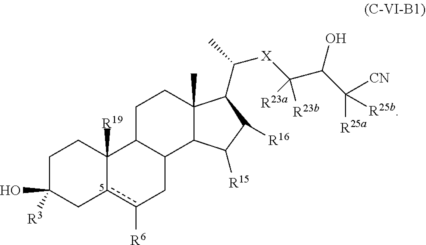

C-91. The compound or pharmaceutically acceptable salt thereof according to embodiment C-90, wherein the compound of Formula (C-VI-B1) is a compound of Formula (C-VI-B1-i):

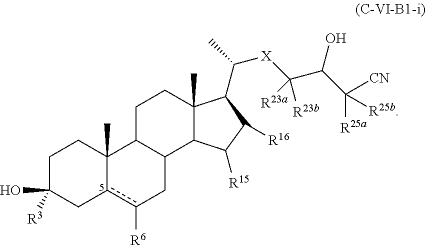

C-92. The compound or pharmaceutically acceptable salt thereof according to embodiment C-91, wherein the compound of Formula (C-VI-B1-i) is a compound of Formula (C-VI-B1-ia):

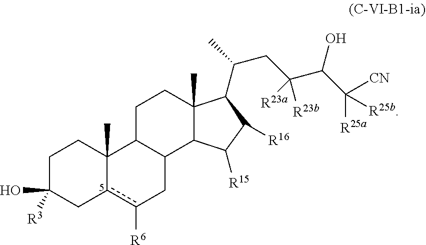

C-93. The compound or pharmaceutically acceptable salt thereof according to embodiment C-92, wherein the compound of Formula (C-VI-B1-ia) is a compound of Formula (C-VI-B1-ia-1):

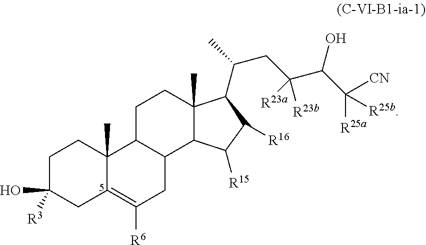

C-94. The compound or pharmaceutically acceptable salt thereof according to embodiment C-93, wherein the compound of Formula (C-VI-B1-ia-1) is a compound of Formula (C-VI-B1-ia-1a) or Formula (C-VI-B1-ia-1b):

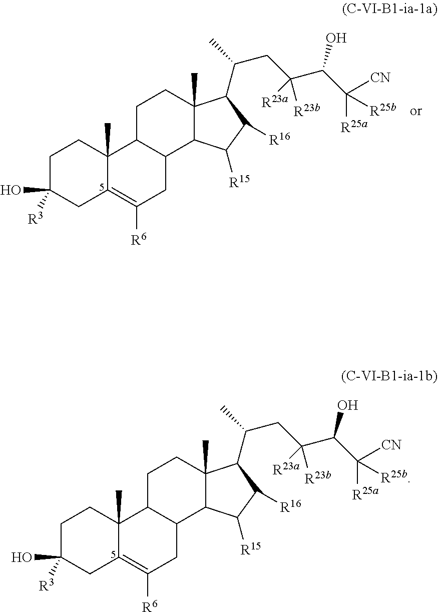

C-95. The compound or pharmaceutically acceptable salt thereof according to embodiment C-92, wherein the compound of Formula (C-VI-B1-ia) is a compound of Formula (C-VI-B1-ia-2):

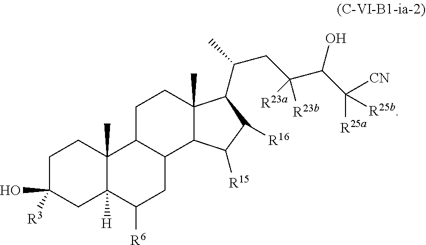

C-96. The compound or pharmaceutically acceptable salt thereof according to embodiment C-95, wherein the compound of Formula (C-VI-B1-ia-2) is a compound of Formula (C-VI-B1-ia-2a) or Formula (C-VI-B1-ia-2b):

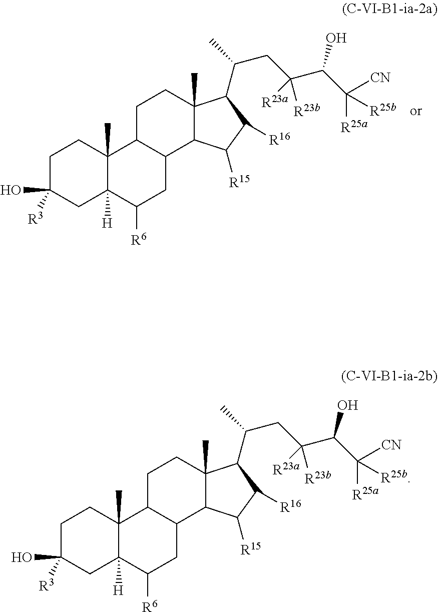

C-97. The compound or pharmaceutically acceptable salt thereof according to embodiment C-90, wherein the compound of Formula (C-VI-B1) is a compound of Formula (C-VI-B1-ii):

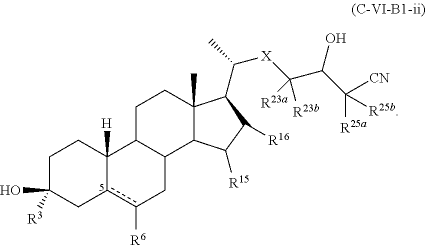

C-98. The compound or pharmaceutically acceptable salt thereof according to embodiment C-97, wherein the compound of Formula (C-VI-B1-ii) is a compound of Formula (C-VI-B1-iia):

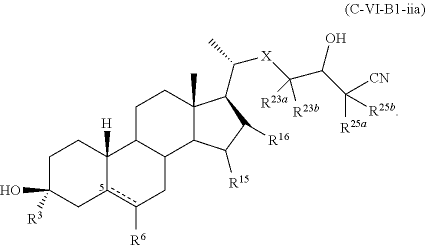

C-99. The compound or pharmaceutically acceptable salt thereof according to embodiment C-98, wherein the compound of Formula (C-VI-B1-iia) is a compound of Formula (C-VI-B1-iia-1):

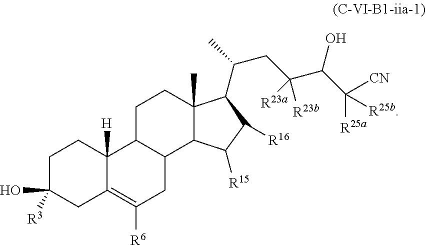

C-100. The compound or pharmaceutically acceptable salt thereof according to embodiment C-99, wherein the compound of Formula (C-VI-B1-iia-1) is a compound of Formula (C-VI-B1-iia-1a) or Formula (C-VI-B1-iia-1b):

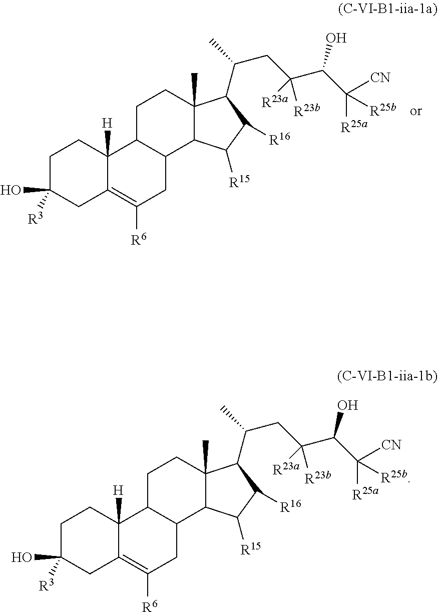

C-101. The compound or pharmaceutically acceptable salt thereof according to embodiment C-98, wherein the compound of Formula (C-VI-B1-iia) is a compound of Formula (C-VI-B1-iia-2):

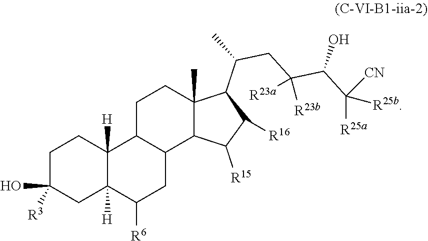

C-102. The compound or pharmaceutically acceptable salt thereof according to embodiment C-101, wherein the compound of Formula (C-VI-B1-iia-2) is a compound of Formula (C-VI-B1-iia-2a) or Formula (C-VI-B1-iia-2b):

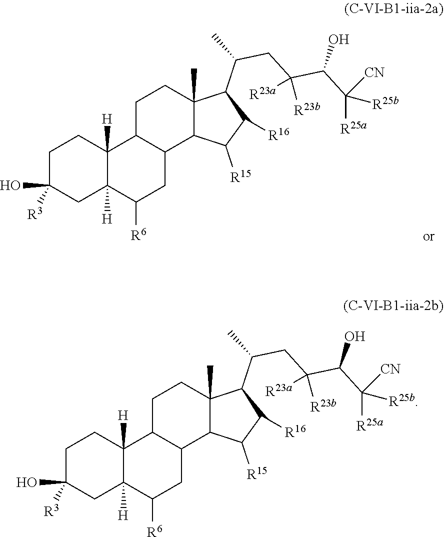

C-103. The compound or pharmaceutically acceptable salt thereof according to embodiments C-1, C-2, C-17, C-32, C-47, and C-75 wherein each of R^{2a} and R^{2b} is hydrogen.

C-104. The compound or pharmaceutically acceptable salt thereof according to embodiments C-1, C-2, C-17, C-32, C-47, and C-75, wherein each of R^{11a} and R^{11b} is hydrogen.

C-105. The compound or pharmaceutically acceptable salt thereof according to any one of embodiments C-1 to C-102, wherein R³ is substituted or unsubstituted C₁₋₆alkyl.

C-106. The compound or pharmaceutically acceptable salt thereof according to embodiment C-105, wherein R³ is —CH₃, —CH₂CH₃, —CH₂CH₂CH₃, or —CH₂OCH₃.

C-107. The compound or pharmaceutically acceptable salt thereof according to embodiment C-106, wherein R³ is —CH₂CH₃.

C-108. The compound or pharmaceutically acceptable salt thereof according to embodiment C-106, wherein R³ is —CH₂OCH₃.

C-109. The compound or pharmaceutically acceptable salt thereof according to any one of embodiments 1-102, wherein R⁶ is hydrogen.

C-110. The compound or pharmaceutically acceptable salt thereof according to any one of embodiments C-1 to C-102, wherein X is —(C(R^{X})₂—, wherein R^{X} is hydrogen or fluorine.

C-111. The compound or pharmaceutically acceptable salt thereof according to embodiment C-110, wherein X is —CH₂—.

C-112. The compound or pharmaceutically acceptable salt thereof according to any one of embodiments C-1 to C-102, wherein each of R^{23a} and R^{23b} is independently hydrogen.

C-113. The compound or pharmaceutically acceptable salt thereof according to any one of embodiments C-1 to C-102, wherein each of R^{23a} and R^{23b} is independently fluorine.

C-114. The compound or pharmaceutically acceptable salt thereof according to any one of embodiments C-1 to C-102, wherein R^{25a} is hydrogen or unsubstituted C₁₋₆ alkyl.

C-115. The compound or pharmaceutically acceptable salt thereof according to embodiment C-114, wherein R^{25a} is hydrogen, —CH₃, or —CH₂CH₃.

C-116. The compound or pharmaceutically acceptable salt thereof according to embodiment C-115, wherein R^{25a} is hydrogen.

C-117. The compound or pharmaceutically acceptable salt thereof according to embodiment C-115, wherein R^{25a} is —CH₃.

C-118. The compound or pharmaceutically acceptable salt thereof according to embodiment C-115, wherein R^{25a} is —CH₂CH₃.

C-119. The compound or pharmaceutically acceptable salt thereof according to any one of embodiments C-1 to C-102, wherein R^{25b} is hydrogen or unsubstituted C₁₋₆ alkyl.

C-120. The compound or pharmaceutically acceptable salt thereof according to embodiment C-119, wherein R^{25b} is hydrogen.

C-121. The compound or pharmaceutically acceptable salt thereof according to embodiment C-119, wherein R^{25b} is —CH₃.

C-122. The compound or pharmaceutically acceptable salt thereof according to any one of embodiments C-1 to C-102, wherein R^{25a} is unsubstituted C₁₋₆alkyl and R 25b is hydrogen or unsubstituted C₁₋₆alkyl.

C-123. The compound or pharmaceutically acceptable salt thereof according to any one of embodiments C-1 to C-102, wherein R^{25a} is hydrogen or unsubstituted C₁₋₆alkyl and R^{25b} is hydrogen or unsubstituted C₁₋₆alkyl.

C-124. The compound or pharmaceutically acceptable salt thereof according to embodiment C-123, wherein R^{25a} is —CH₃ and R^{25b} is —CH₃.

C-125. The compound or pharmaceutically acceptable salt thereof according to embodiment C-123, wherein R^{25a} is —CH₂CH₃ and R^{25b} is hydrogen.

C-126. The compound or pharmaceutically acceptable salt thereof according to embodiment C-123, wherein R^{25a} is hydrogen and R^{25b} is hydrogen.

C-127. The compound or pharmaceutically acceptable salt thereof according to any one of embodiments C-1 to C-102, wherein R^{25a} and R^{25b} taken together with the carbon atom to which they are attached, form an unsubstituted C₃₋₆carbocyclyl.

C-128. The compound or pharmaceutically acceptable salt thereof according to embodiment C-127, wherein R^{25a} and R^{25b} taken together with the carbon atom to which they are attached, form a cyclopropyl.

C-129. The compound or pharmaceutically acceptable salt thereof according to any one of embodiments C-1 to C-7, C-11 to C-12, C-17 to C-21, C-26 to C-27, C-32 to C-36, C-41 to C-42, C-47 to C-51, C-56 to C-57, C-62 to C-64, C-69 to C-70, C-75 to C-79, C-84 to C-85, C-90 to C-92, and C-97 to C-98, wherein the bond between C5 and C6 is a single bond and R⁵ is hydrogen in the alpha configuration.

C-130. The compound or pharmaceutically acceptable salt thereof according to any one of embodiments C-1, C-47 and C-75, wherein the bond between C9 and C11 is a double bond, R⁹ is absent and R^{11a} is selected from the group consisting of hydrogen, halogen, substituted or unsubstituted C₁₋₆alkyl, and OR^{A11}, wherein R^{A11} is hydrogen or substituted or unsubstituted C₁₋₆alkyl, and R^{11b} is absent or hydrogen.

C-131. The compound or pharmaceutically acceptable salt thereof according to any one of embodiments C-1, C-47 and C-75, wherein the bond between C9 and C11 is a single bond, R⁹ is hydrogen and R¹¹ is selected from the group consisting of halogen or substituted or unsubstituted C₁₋₆alkyl.

C-132. The compound or pharmaceutically acceptable salt thereof according to any one of embodiments C-1 and C-47 to C-102, wherein each instance of R¹⁵ is independently hydrogen, substituted or unsubstituted C₁₋₆alkyl or substituted or unsubstituted C₃₋₆carbocyclyl.

C-133. The compound or pharmaceutically acceptable salt thereof according to embodiment C-132, wherein each instance of R¹⁵ is independently hydrogen, unsubstituted C₁₋₆alkyl or unsubstituted C₃₋₆carbocyclyl.

C-134. The compound or pharmaceutically acceptable salt thereof according to embodiment C-133, wherein each instance of R¹⁵ is independently hydrogen, —CH₃ or cyclopropyl.

C-135. The compound or pharmaceutically acceptable salt thereof according to embodiment C-134, wherein each instance of R¹⁵ is independently hydrogen.

C-136. The compound or pharmaceutically acceptable salt thereof according to embodiment C-134, wherein each instance of R¹⁵ is independently —CH₃.

C-137. The compound or pharmaceutically acceptable salt thereof according to embodiment C-134, wherein each instance of R¹⁵ is independently cyclopropyl.

C-138. The compound or pharmaceutically acceptable salt thereof according to any one of embodiments C-1 and C-47 to C-102, wherein R¹⁶ is hydrogen, substituted or unsubstituted C₁₋₆alkyl or substituted or unsubstituted C₃₋₆carbocyclyl.

C-139. The compound or pharmaceutically acceptable salt thereof according to embodiment C-138, wherein R¹⁶ is hydrogen or unsubstituted C₁₋₆alkyl.

C-140. The compound or pharmaceutically acceptable salt thereof according to embodiment C-138, wherein R¹⁶ is hydrogen.

C-141. The compound or pharmaceutically acceptable salt thereof according to embodiment C-138, wherein R¹⁶ is —CH₃.

C-142. The compound or pharmaceutically acceptable salt thereof according to any one of embodiments C-1 and C-47 to C-102, wherein R¹⁵ and R¹⁶ are taken together to form a substituted or unsubstituted C₃₋₆carbocyclyl.

C-143. The compound or pharmaceutically acceptable salt thereof according to embodiment C-142, wherein R¹⁵ and R¹⁶ are taken together to form a cyclopropyl.

C-144. The compound or pharmaceutically acceptable salt thereof according to any one of embodiments C-1, C-2, C-17, C-18, C-32, C-33, C-47, C-48, C-75 and C-76, wherein R¹⁸ is hydrogen, unsubstituted C₁₋₆alkyl or C₁₋₆alkyl substituted with OR^{A18}, wherein R^{A18} is substituted or unsubstituted C₁₋₆alkyl.

C-145. The compound or pharmaceutically acceptable salt thereof according to any one of embodiments C-1, C-2, C-17, C-18, C-32, C-33, C-47, C-48, C-75 and C-76, wherein R¹⁸ is hydrogen, —CH₃, CH₂OCH₃, —OCH₃, —CH₂CH₃, or —CH(CH₃)₂.

C-146. The compound or pharmaceutically acceptable salt thereof according to embodiment C-145, wherein R¹⁸ is hydrogen, —CH₂CH₃ or —CH₃.

C-147. The compound or pharmaceutically acceptable salt thereof according to embodiment C-146, wherein R¹⁸ is —CH₂CH₃.

C-148. The compound or pharmaceutically acceptable salt thereof according to embodiment C-146, wherein R¹⁸ is —CH₃.

C-149. The compound or pharmaceutically acceptable salt thereof according to embodiment C-146, wherein R¹⁸ is hydrogen.

C-150. The compound or pharmaceutically acceptable salt thereof according to any one of embodiments C-1 to C-4, C-17 to C-19, C-32 to C-34, C-47 to C-49, and C-75 to C-77, wherein R¹⁹ is hydrogen, unsubstituted C₁₋₆alkyl or C₁₋₆alkyl substituted with OR^{A19} wherein R^{A19} is substituted or unsubstituted C₁₋₆alkyl.

C-151. The compound or pharmaceutically acceptable salt thereof according to embodiment C-150, wherein R¹⁹ is hydrogen, —CH₃, CH₂OCH₃, —OCH₃, —CH₂CH₃, or —CH(CH₃)₂.

C-152. The compound or pharmaceutically acceptable salt thereof according to embodiment C-151, wherein R¹⁹ is hydrogen or —CH₃.

C-153. The compound or pharmaceutically acceptable salt thereof according to embodiment C-152, wherein R¹⁹ is —CH₃.

C-154. The compound or pharmaceutically acceptable salt thereof according embodiment C-152, wherein R¹⁹ is hydrogen.

C-155. The compound or pharmaceutically acceptable salt thereof according to embodiment C-1, wherein the compound of Formula (C-I) is selected from the group consisting of Compounds C1-C35 and pharmaceutically acceptable salts thereof.

C-156. The compound or pharmaceutically acceptable salt thereof according to embodiment C-1, wherein the compound of Formula (C-I) is selected from the group consisting of Compounds C1-C17 and pharmaceutically acceptable salts thereof.

C-157. The compound or pharmaceutically acceptable salt thereof according to embodiment C-1, wherein the compound of Formula (C-I) is selected from the group consisting of C-1, C-2, C-3, C-5, C-7, C-8, C-9, C-11 and pharmaceutically acceptable salts thereof.

C-158. A pharmaceutical composition comprising a compound or pharmaceutically acceptable salt thereof according to any one of embodiments C-1 to C-157, and a pharmaceutically acceptable carrier.

C-159. A method for treating a CNS-related condition in a subject, comprising administering to the subject an effective amount of a compound or pharmaceutically acceptable salt thereof according to any one of embodiments C-1 to C-157, or a pharmaceutical composition according to embodiment C-158.

C-160. The method according to embodiment C-159, wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease) and tinnitus.

C-161. A method of inducing sedation or anesthesia in a subject, comprising administering to the subject an effective amount of a compound or pharmaceutically acceptable salt thereof according to any one of embodiments C-1 to C-157, or a pharmaceutical composition according to embodiment C-158.

C-162. A compound or pharmaceutically acceptable salt thereof according to any one of embodiments C-1 to C-157, or a pharmaceutical composition according to embodiment C-158, for use in treating a CNS-related condition in a subject.

C-163. The compound or pharmaceutically acceptable salt thereof, or pharmaceutical composition for use according to embodiment C-162, wherein the wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease) and tinnitus.

C-164. A compound or pharmaceutically acceptable salt thereof according to any one of embodiments C-1 to C-157, or a pharmaceutical composition according to embodiment C-158, for use inducing sedation or anesthesia in a subject.

C-165. Use of a compound or pharmaceutically acceptable salt thereof according to any one of embodiments C-1 to C-157, or a pharmaceutical composition according to embodiment C-158, for the manufacture of a medicament for treating a CNS-related condition in a subject.

C-166. The use according to embodiment C-165, wherein the wherein the wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease) and tinnitus.

C-167. Use of a compound or pharmaceutically acceptable salt thereof according to any one of embodiments C-1 to C-157, or a pharmaceutical composition according to embodiment C-158, for the manufacture of a medicament for inducing sedation or anesthesia in a subject.

C-168. A compound of Formula (C-I):

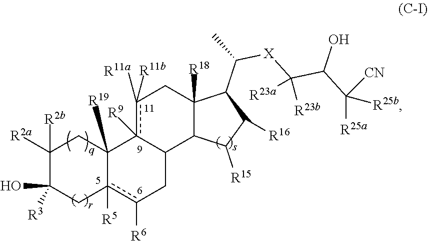

wherein:
   each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein R^{A2} is hydrogen or substituted or unsubstituted C₁₋₆alkyl;
   R³ is substituted or unsubstituted C₁₋₆alkyl, substituted or unsubstituted C₂₋₆ alkenyl, or substituted or unsubstituted C₂₋₆alkynyl, or substituted or unsubstituted C₃₋₁₀carbocyclyl;
   R⁵ is absent, hydrogen, halogen, or substituted or unsubstituted C₁₋₆alkyl;
   R⁶ is hydrogen, halogen, or substituted or unsubstituted C₁₋₆alkyl;
   R⁹ is absent or hydrogen; or
   R⁹ and R^{11a} are taken together with the carbon atoms to which they are attached to form a substituted or unsubstituted C₃₋₆carbocyclyl ring;
   R^{11a} is hydrogen, halogen, substituted or unsubstituted C₁₋₆alkyl, or —OR^{A11}, wherein R^{A11} is hydrogen or substituted or unsubstituted C₁₋₆alkyl;
   R^{11b} is absent or hydrogen; or
   R^{11a} and R^{11b} are joined to form an oxo (═O) group;
   each instance of R¹⁵ is hydrogen, halogen, —OH, —NH₂, substituted or unsubstituted C₁₋₆alkyl, substituted or unsubstituted C₂₋₆alkenyl, substituted or unsubstituted C₂₋₆alkynyl, substituted or unsubstituted C₁₋₆alkoxy, substituted or unsubstituted C₃₋₆carbocyclyl, or substituted or unsubstituted 3- to 10-membered heterocyclyl;
   R¹⁶ is hydrogen, halogen, —OH, —NH₂, substituted or unsubstituted C₁₋₆alkyl, substituted or unsubstituted C₂₋₆alkenyl, substituted or unsubstituted C₂₋₆alkynyl, substituted or unsubstituted C₁₋₆alkoxy, substituted or unsubstituted C₃₋₆carbocyclyl, or substituted or unsubstituted 3- to 10-membered heterocyclyl; or
   R¹⁵ and R¹⁶ are taken together with the carbon atoms to which they are attached, to form a substituted or unsubstituted C₃₋₆carbocyclyl;
   R¹⁸ is hydrogen, substituted or unsubstituted C₁₋₆alkyl, or OR^{A18}, wherein each instance of R^{A18} is independently substituted or unsubstituted C₁₋₆alkyl;
   R¹⁹ is hydrogen, substituted or unsubstituted C₁₋₆alkyl, or OR^{A19}; wherein each instance of R^{A19} is independently substituted or unsubstituted C₁₋₆alkyl;
   X is —(C(R^{X})₂)ₙ— or —O—, wherein R^{X} is hydrogen or fluorine, or one R^{X} group and R^{23b} are joined to form a double bond;
   n is 1, 2 or 3;
   each of R^{23a} and R^{23b} is independently hydrogen or fluorine;
   R^{25a} is hydrogen, substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, or substituted or unsubstituted 5- to 10-membered heteroaryl;
   R^{25b} is hydrogen, substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, or substituted or unsubstituted 5- to 10-membered heteroaryl;
   or R^{25a} and R^{25b}, taken together with the carbon atom to which they are attached, form a substituted or unsubstituted C₃₋₆carbocyclyl or substituted or unsubstituted 3- to 10-membered heterocyclyl;
   q is 0, 1 or 2;
   r is 1 or 2;
   s is 1 or 2; and
   each [symbol] independently represents a single or double bond;
   provided if the bond between C5 and C6 is a double bond, R⁵ is absent and if the bond between C9 and C11 is a double bond, R⁹ and R^{11b} are absent.

C-169. The compound according to embodiment C-168, wherein the compound of Formula (C-I) is a compound of Formula (C-II):

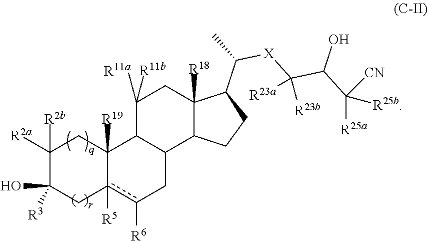

C-170. The compound according to embodiment C-169, wherein the compound of Formula (C-II) is a compound of Formula (C-II-A):

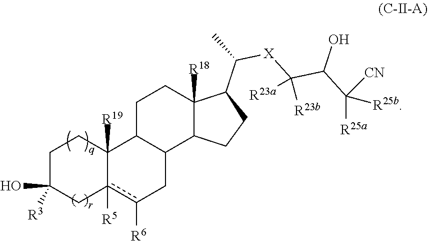

C-171. The compound according to embodiment C-170, wherein the compound of Formula (C-II-A) is a compound of Formula (C-II-A1):

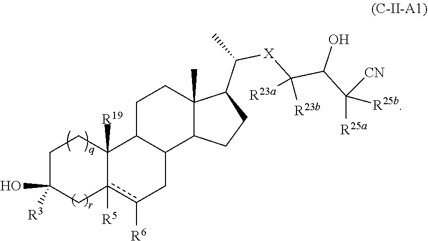

C-172. The compound according to embodiment C-171, wherein the compound of Formula (C-II-A1) is a compound of Formula (C-II-A1-i):

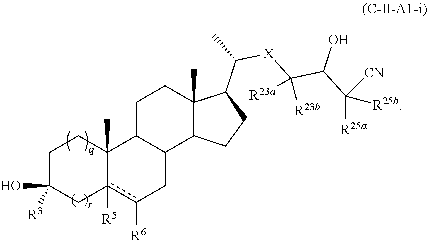

C-173. The compound according to embodiment C-172, wherein the compound of Formula (C-II-A1-i) is a compound of Formula (C-II-A1-ia):

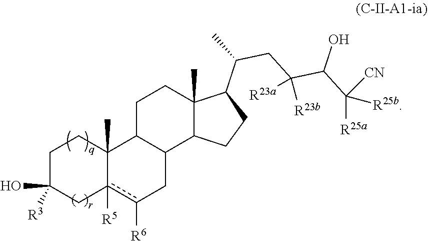

C-174. The compound according to embodiment C-173, wherein the compound of Formula (C-II-A1-ia) is a compound of Formula (C-II-A1-ia-1):

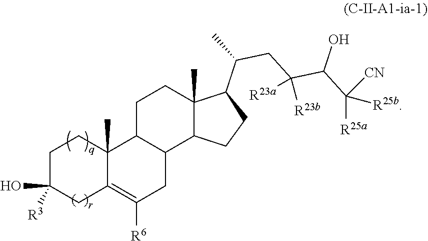

C-175. The compound according to embodiment C-174, wherein the compound of Formula (C-II-A1-ia-1) is a compound of Formula (C-II-A1-ia-1a) or Formula (I C-I-A1-ia-1b):

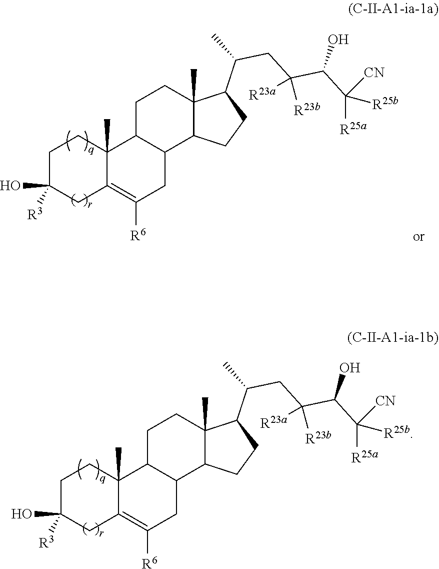

C-176. The compound according to embodiment C-173, wherein the compound of Formula (C-II-A1-ia) is a compound of Formula (C-II-A1-ia-2):

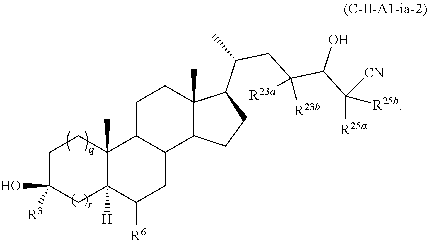

C-177. The compound according to embodiment C-176, wherein the compound of Formula (C-II-A1-ia-2) is a compound of Formula (C-II-A1-ia-2a) or Formula (C-II-A1-ia-2b):

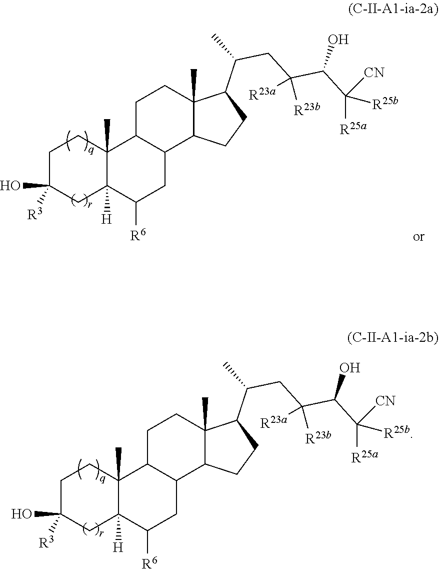

C-178. The compound according to embodiment C-171, wherein the compound of Formula (C-II-A1) is a compound of Formula (C-II-A1-ii):

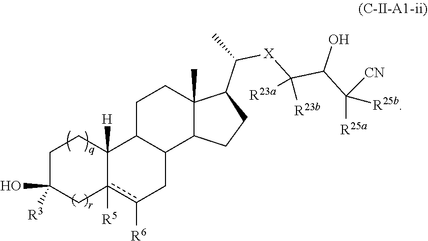

C-179. The compound according to embodiment C-178, wherein the compound of Formula (C-II-A1-ii) is a compound of Formula (C-II-A1-iia):

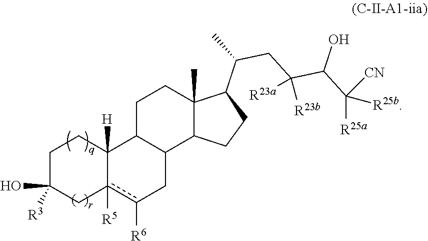

C-180. The compound according to embodiment C-179, wherein the compound of Formula (C-II-A1-iia) is a compound of Formula (C-II-A1-iia-1):

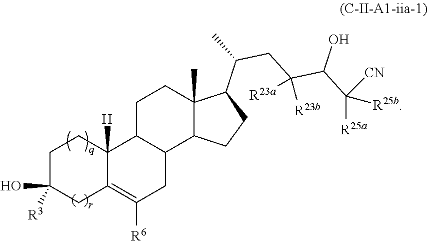

C-181. The compound according to embodiment C-180, wherein the compound of Formula (C-II-A1-iia-1) is a compound of Formula (C-II-A1-iia-1a) or Formula (C-II-A1-iia-1b):

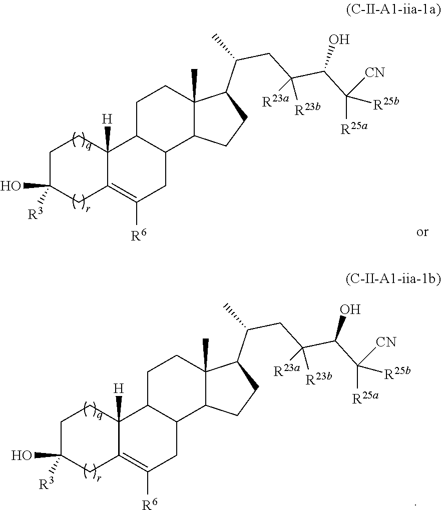

C-182. The compound according to embodiment C-179, wherein the compound of Formula (C-II-A1-iia) is a compound of Formula (C-II-A1-iia-2):

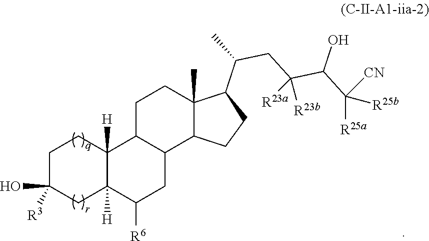

C-183. The compound according to embodiment C-182, wherein the compound of Formula (C-II-A1-iia-2) is a compound of Formula (C-II-A1-iia-2a) or Formula (C-II-A1-iia-2b):

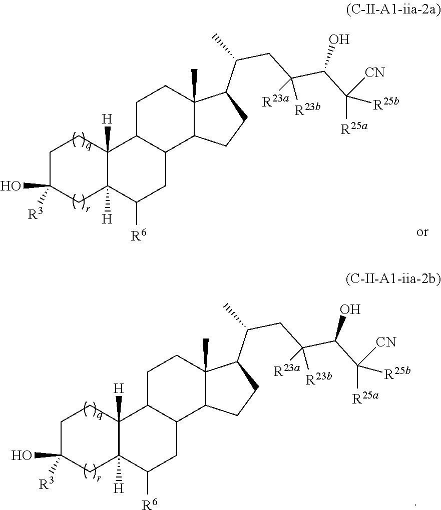

C-184. The compound according to embodiment C-168, wherein the compound of Formula (C-I) is a compound of Formula (C-III):

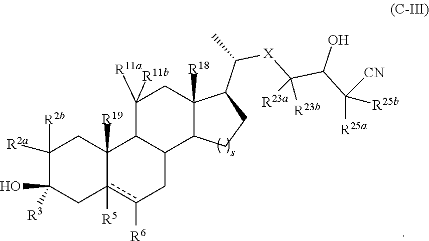

C-185. The compound according to embodiment C-184, wherein the compound of Formula (C-III) is a compound of Formula (C-III-A):

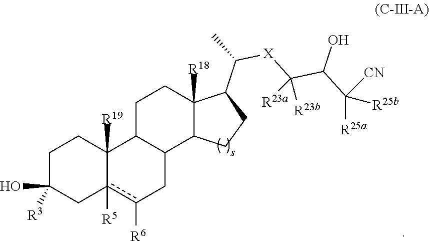

C-186. The compound according to embodiment C-185, wherein the compound of Formula (C-III-A) is a compound of Formula (C-III-A1):

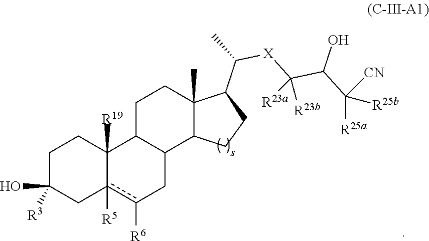

C-187. The compound according to embodiment C-186, wherein the compound of Formula (C-III-A1) is a compound of Formula (C-III-A1-i):

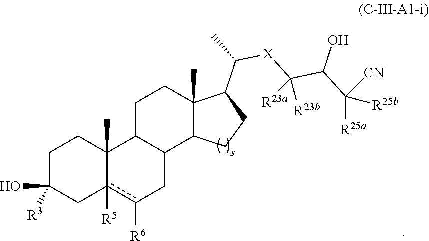

C-188. The compound according to embodiment C-187, wherein the compound of Formula (C-III-A1-i) is a compound of Formula (C-III-A1-ia):

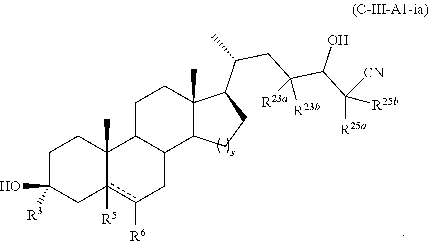

C-189. The compound according to embodiment C-188, wherein the compound of Formula (C-III-A1-ia) is a compound of Formula (C-III-A1-ia-1):

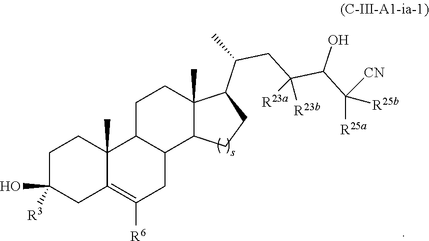

C-190. The compound according to embodiment C-189, wherein the compound of Formula (C-III-A1-ia-1) is a compound of Formula (C-III-A1-ia-1a) or Formula (C-III-A1-ia-1b):

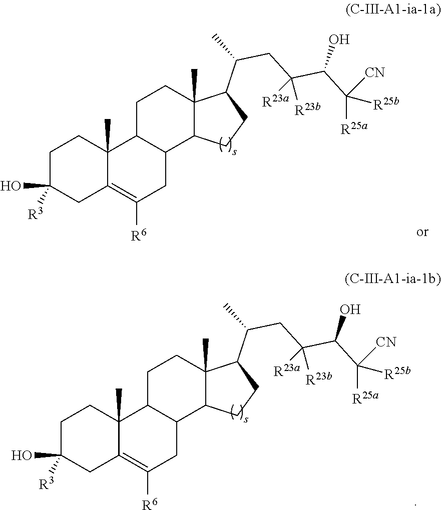

C-191. The compound according to embodiment C-188, wherein the compound of Formula (C-III-A1-ia) is a compound of Formula (C-III-A1-ia-2):

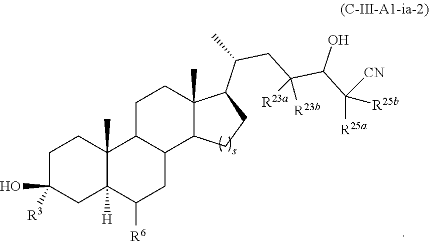

C-192. The compound according to embodiment C-191, wherein the compound of Formula (C-III-A1-ia-2) is a compound of Formula (C-III-A1-ia-2a) or Formula (C-III-A1-ia-2b):

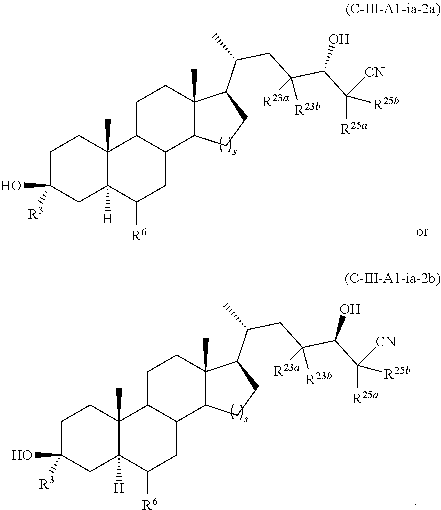

C-193. The compound according to embodiment C-186, wherein the compound of Formula (C-III-A1) is a compound of Formula (C-III-A1-ii):

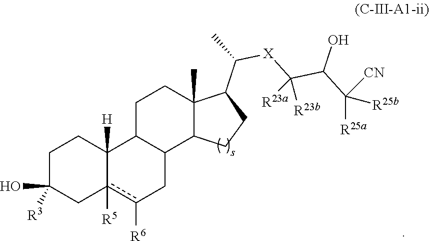

C-194. The compound according to embodiment C-193, wherein the compound of Formula (C-III-A1-ii) is a compound of Formula (C-III-A1-iia):

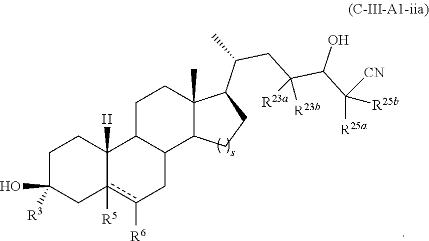

C-195. The compound according to embodiment C-194, wherein the compound of Formula (C-III-A1-iia) is a compound of Formula (C-III-A1-iia-1):

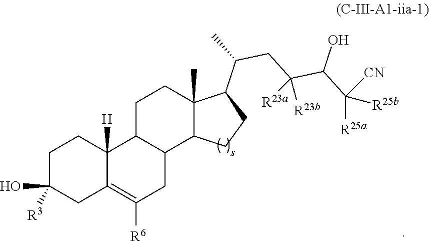

C-196. The compound according to embodiment C-195, wherein the compound of Formula (C-III-A1-iia-1) is a compound of Formula (C-III-A1-iia-1a) or Formula (C-III-A1-iia-1b):

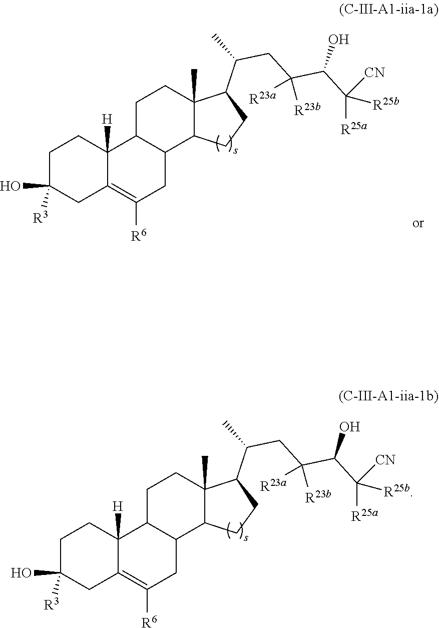

C-197. The compound according to embodiment C-194, wherein the compound of Formula (C-III-A1-iia) is a compound of Formula (C-III-A1-iia-2):

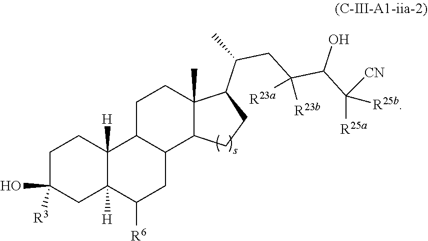

C-198. The compound according to embodiment C-197, wherein the compound of Formula (C-III-A1-iia-2) is a compound of Formula (C-III-A1-iia-2a) or Formula (C-III-A1-iia-2b):

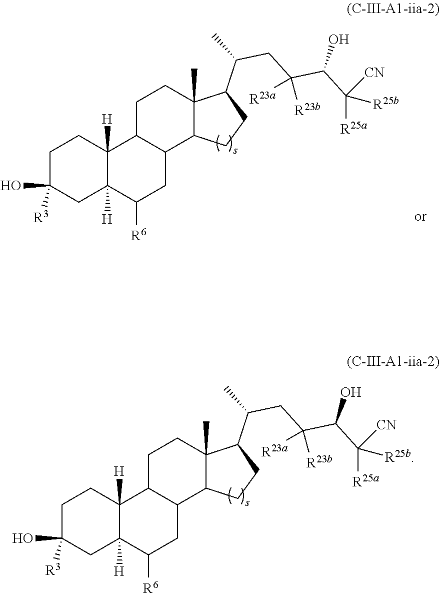

C-199. The compound according to embodiment C-168, wherein the compound of Formula (I) is a compound of Formula (C-IV):

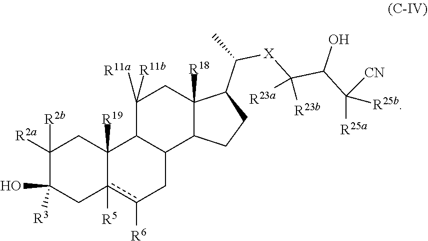

C-200. The compound according to embodiment C-199, wherein the compound of Formula (C-IV) is a compound of Formula (C-IV-A):

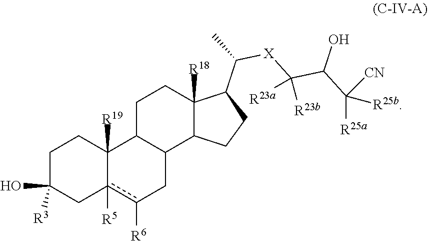

C-201. The compound according to embodiment C-200, wherein the compound of Formula (C-IV-A) is a compound of Formula (C-IV-A1):

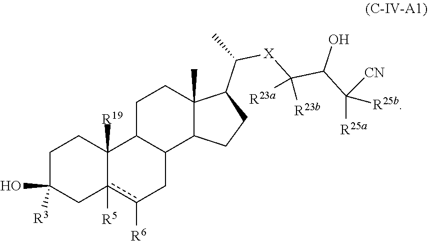

C-202. The compound according to embodiment C-201, wherein the compound of Formula (C-IV-A1) is a compound of Formula (C-IV-A1-i):

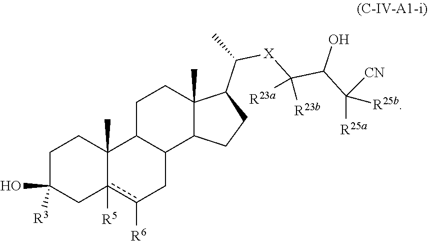

C-203. The compound according to embodiment C-202, wherein the compound of Formula (C-IV-A1-i) is a compound of Formula (C-IV-A1-ia):

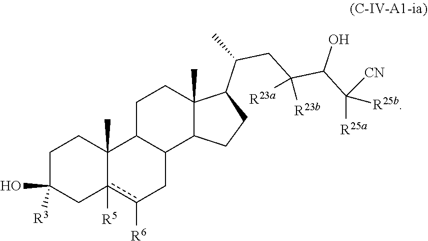

C-204. The compound according to embodiment C-203, wherein the compound of Formula (C-IV-A1-ia) is a compound of Formula (C-IV-A1-ia-1):

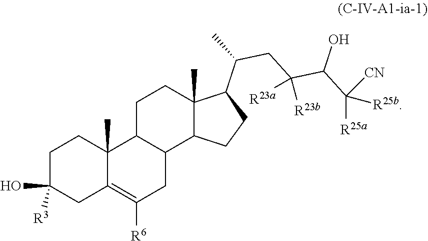

C-205. The compound according to embodiment C-204, wherein the compound of Formula (C-IV-A1-ia-1) is a compound of Formula (C-IV-A1-ia-1a) or Formula (C-IV-A1-ia-1b):

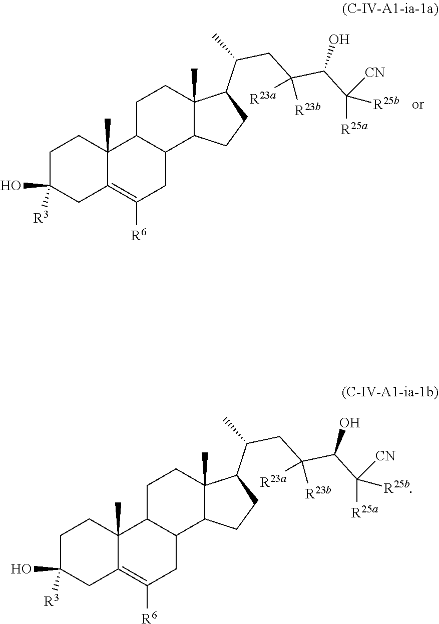

C-206. The compound according to embodiment C-203, wherein the compound of Formula (C-IV-A1-ia) is a compound of Formula (C-IV-A1-ia-2):

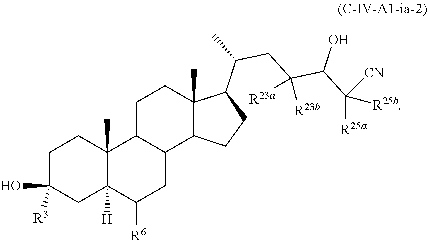

C-207. The compound according to embodiment C-206, wherein the compound of Formula (C-IV-A1-ia-2) is a compound of Formula (C-IV-A1-ia-2a) or Formula (C-IV-A1-ia-2b):

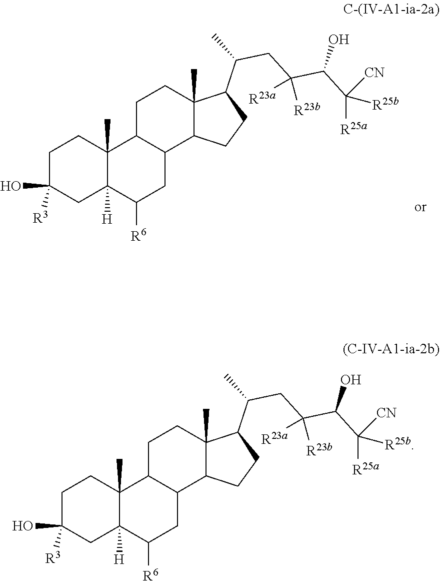

C-208. The compound according to embodiment C-201, wherein the compound of Formula (C-IV-A1) is a compound of Formula (C-IV-A1-ii):

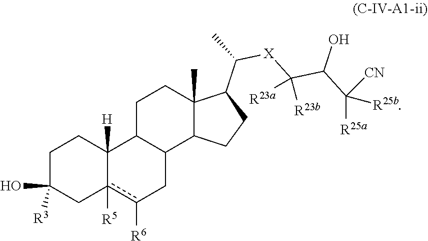

C-209. The compound according to embodiment C-208, wherein the compound of Formula (C-IV-A1-ii) is a compound of Formula (C-IV-A1-iia):

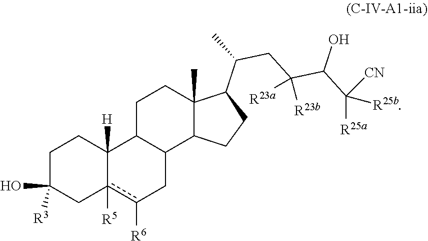

C-210. The compound according to embodiment C-209, wherein the compound of Formula (C-IV-A1-iia) is a compound of Formula (C-IV-A1-iia-1):

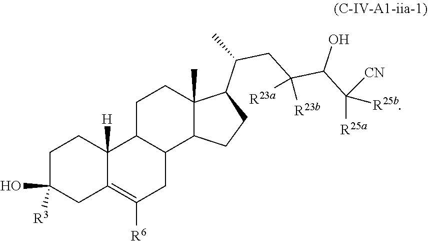

C-211. The compound according to embodiment C-210, wherein the compound of Formula (C-IV-A1-iia-1) is a compound of Formula (C-IV-A1-iia-1a) or Formula (C-IV-A1-iia-1b):

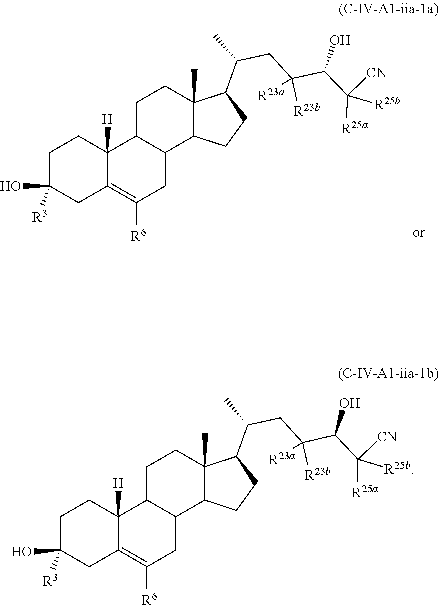

C-212. The compound according to embodiment C-209, wherein the compound of Formula (C-IV-A1-iia) is a compound of Formula (C-IV-A1-iia-2):

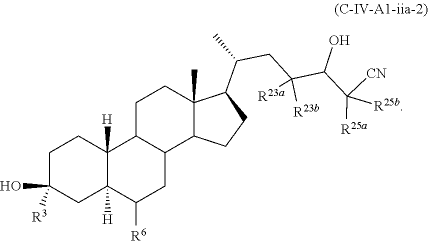

C-213. The compound according to embodiment C-212, wherein the compound of Formula (C-IV-A1-iia-2) is a compound of Formula (C-IV-A1-iia-2a) or Formula (C-IV-A1-iia-2b):

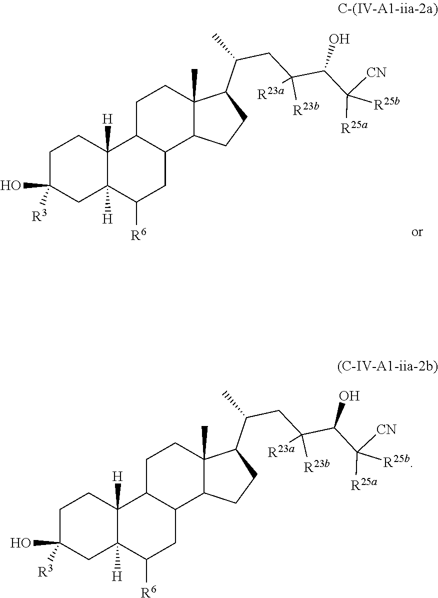

C-214. The compound according to embodiment C-168, wherein the compound of Formula (C-I) is a compound of Formula (C-V):

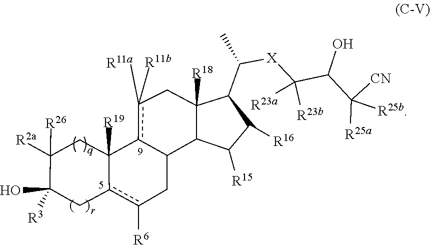

C-215. The compound according to embodiment C-214, wherein the compound of Formula (C-V) is a compound of Formula (C-VA) or Formula (C-VB):

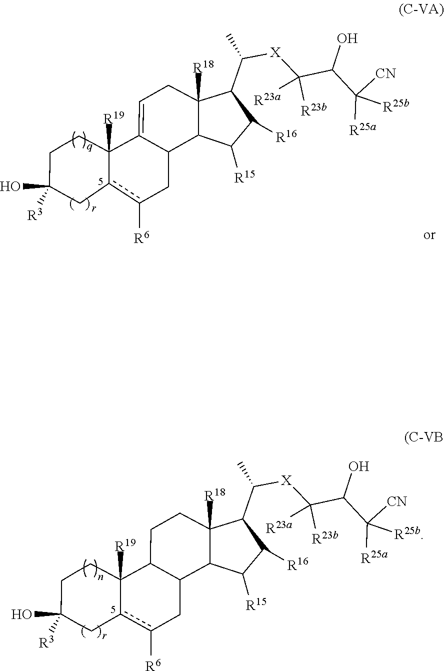

C-216. The compound according to embodiment C-215, wherein the compound of Formula (C-V-A) is a compound of Formula (C-V-A1):

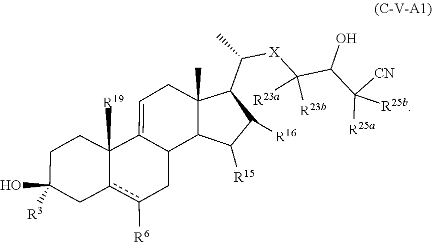

C-217. The compound according to embodiment C-216, wherein the compound of Formula (C-V-A1) is a compound of Formula (C-V-A1-i):

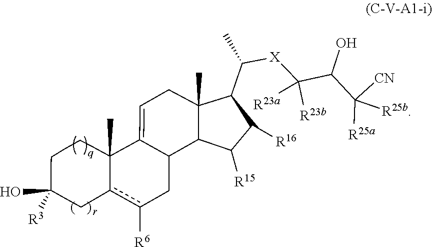

C-218. The compound according to embodiment C-217, wherein the compound of Formula C-(V-A1-i) is a compound of Formula (C-V-A1-ia):

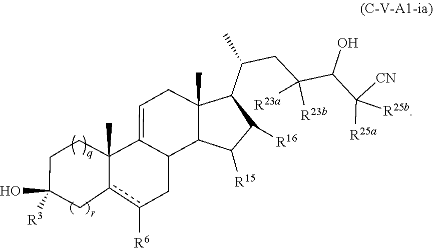

C-219. The compound according to embodiment C-218, wherein the compound of Formula (C-V-A1-ia) is a compound of Formula (C-V-A1-ia-1):

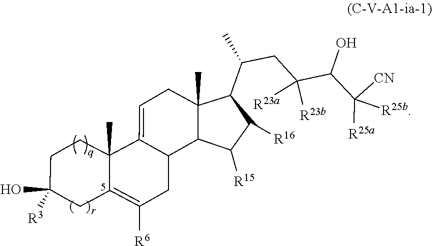

C-220. The compound according to embodiment C-219, wherein the compound of Formula (C-V-A1-ia-1) is a compound of Formula (C-V-A1-ia-1a) or Formula (C-V-A1-ia-1b):

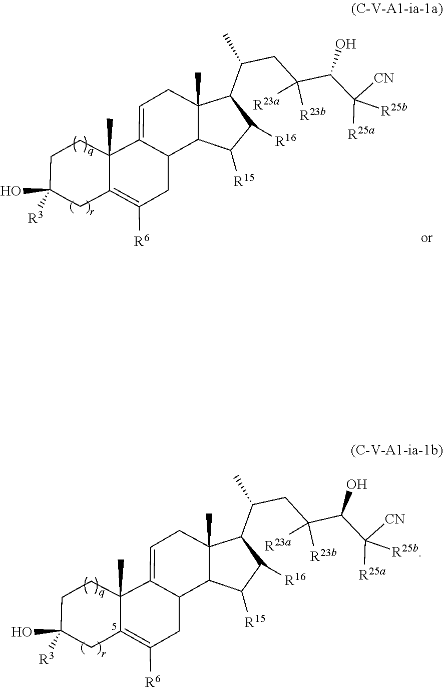

C-221. The compound according to embodiment C-218, wherein the compound of Formula (C-V-A1-ia) is a compound of Formula (C-V-A1-ia-2):

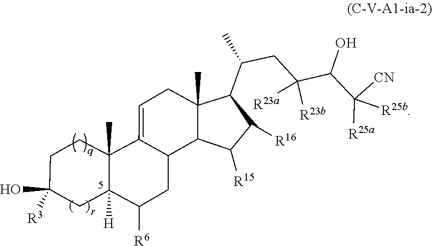

C-222. The compound according to embodiment C-221, wherein the compound of Formula (C-V-A1-ia-2) is a compound of Formula (C-V-A1-ia-2a) or Formula (C-V-A1-ia-2b):

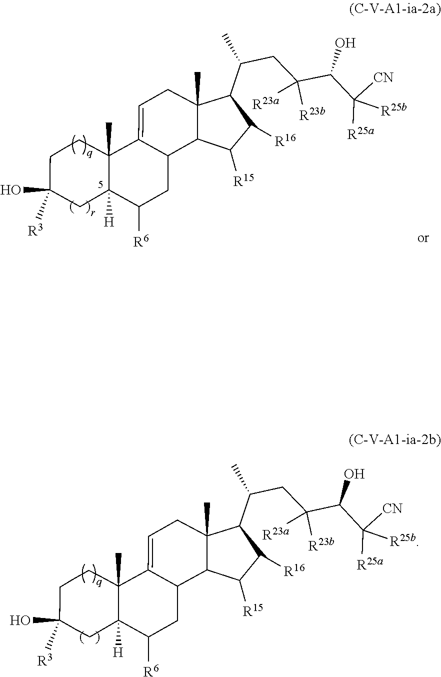

C-223. The compound according to embodiment C-216, wherein the compound of Formula (C-V-A1) is a compound of Formula (C-V-A1-ii):

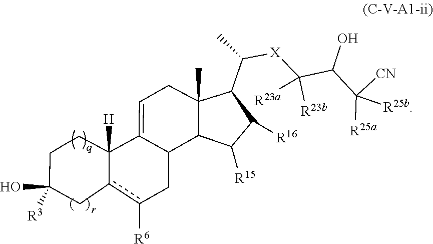

C-224. The compound according to embodiment C-223, wherein the compound of Formula (C-V-A1-ii) is a compound of Formula (C-V-A1-iia):

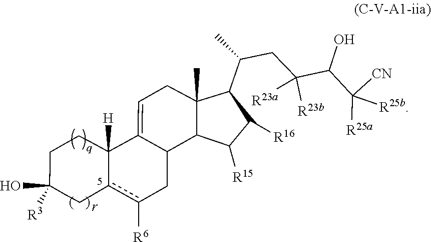

C-225. The compound according to embodiment C-224, wherein the compound of Formula (C-V-A1-iia) is a compound of Formula (C-V-A1-iia-1):

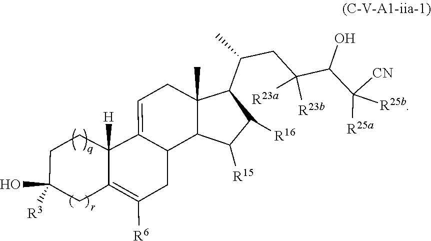

C-226. The compound according to embodiment C-225, wherein the compound of Formula (C-V-A1-iia-1) is a compound of Formula (C-V-A1-iia-1a) or Formula (C-V-A1-iia-1b):

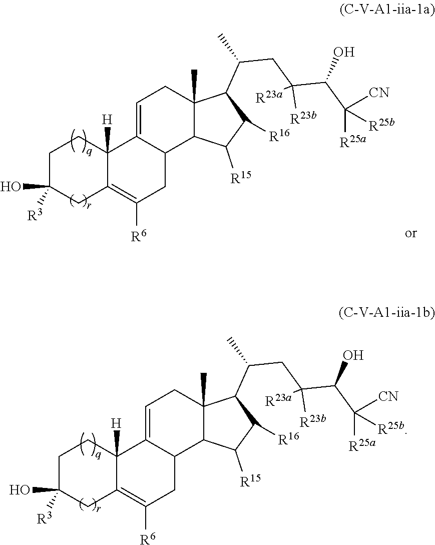

C-227. The compound according to embodiment C-224, wherein the compound of Formula (C-V-A1-iia) is a compound of Formula (C-V-A1-iia-2):

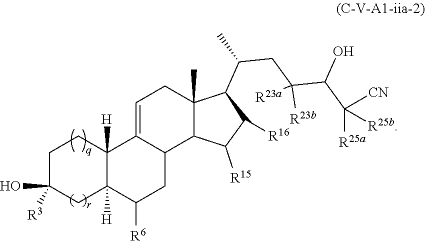

C-228. The compound according to embodiment C-227, wherein the compound of Formula (C-V-A1-iia-2) is a compound of Formula (C-V-A1-iia-2a) or Formula (C-V-A1-iia-2b):

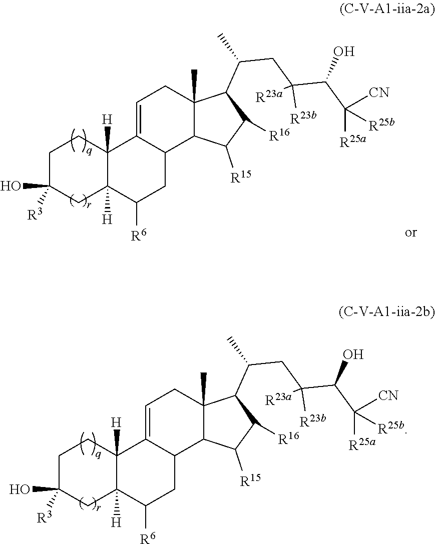

C-229. The compound according to embodiment C-215, wherein the compound of Formula (C-V-B) is a compound of Formula (C-V-B1):

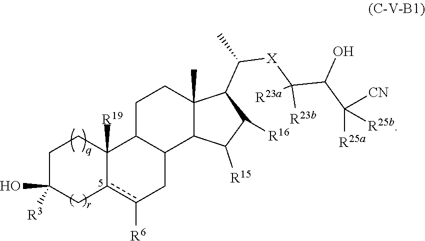

C-230. The compound according to embodiment C-229, wherein the compound of Formula (C-V-B1) is a compound of Formula (C-V-B1-i):

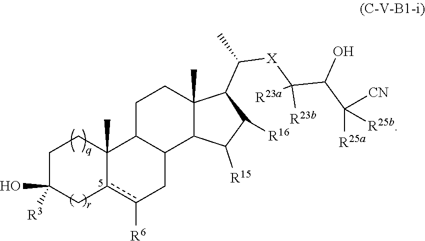

C-231. The compound according to embodiment C-230, wherein the compound of Formula (C-V-B1-i) is a compound of Formula (C-V-B1-ia):

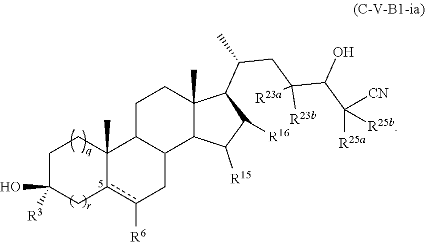

C-232. The compound according to embodiment C-231, wherein the compound of Formula (C-V-B1-ia) is a compound of Formula (C-V-B1-ia-1):

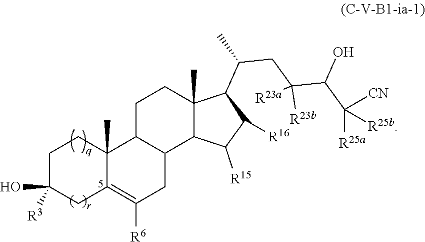

C-233. The compound according to embodiment C-232, wherein the compound of Formula (C-V-B1-ia-1) is a compound of Formula (C-V-B1-ia-1a) or Formula (C-V-B1-ia-1b):

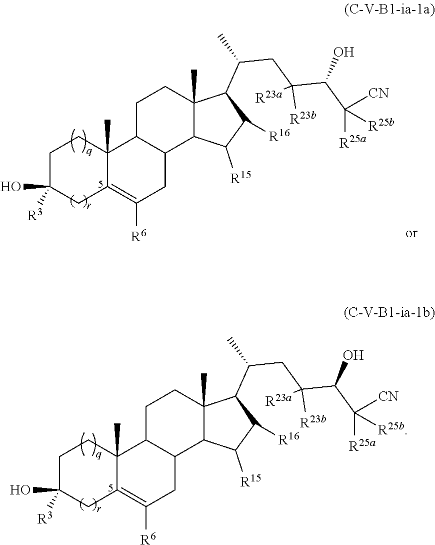

C-234. The compound according to embodiment C-231, wherein the compound of Formula (C-V-B1-ia) is a compound of Formula (C-V-B1-ia-2):

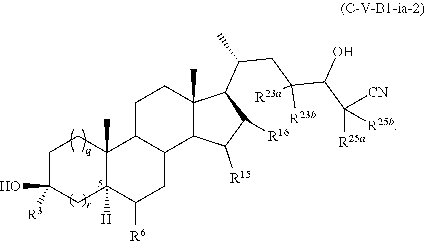

C-235. The compound according to embodiment C-234, wherein the compound of Formula (C-V-B1-ia-2) is a compound of Formula (C-V-B1-ia-2a) or Formula (C-V-B1-ia-2b):

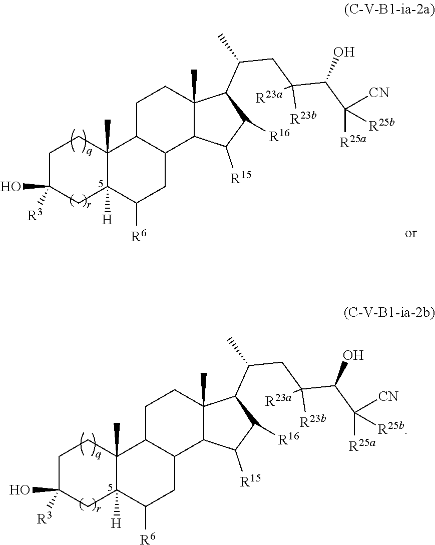

C-236. The compound according to embodiment C-229, wherein the compound of Formula (C-V-B1) is a compound of Formula (C-V-B1-ii):

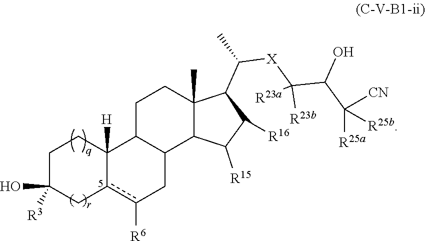

C-237. The compound according to embodiment C-236, wherein the compound of Formula (C-V-B1-ii) is a compound of Formula (C-V-B1-iia):

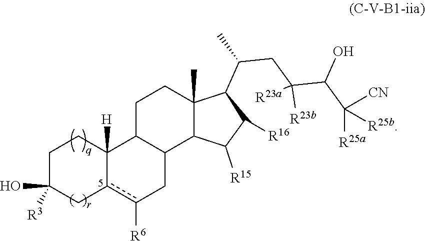

C-238. The compound according to embodiment C-237, wherein the compound of Formula (C-V-B1-iia) is a compound of Formula (C-V-B1-iia-1):

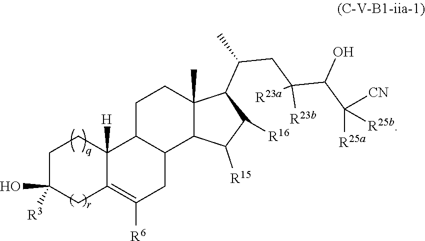

C-239. The compound according to embodiment C-238, wherein the compound of Formula (C-V-B1-iia-1) is a compound of Formula (C-V-B1-iia-1a) or Formula (C-V-B1-iia-1b):

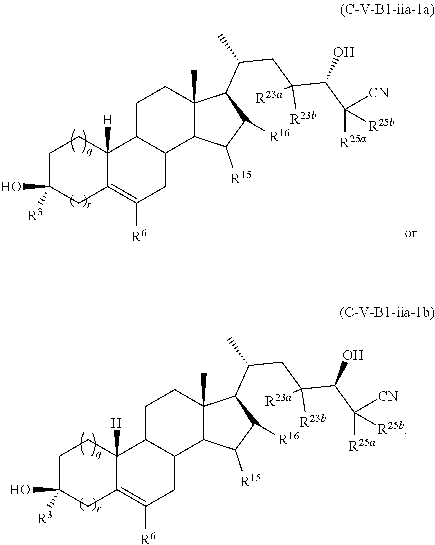

C-240. The compound according to embodiment C-237, wherein the compound of Formula (C-V-B1-iia) is a compound of Formula (C-V-B1-iia-2):

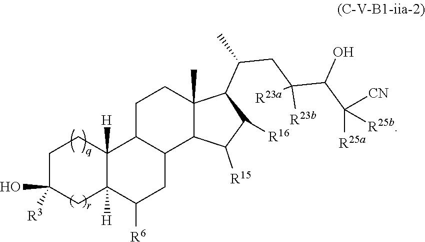

C-241. The compound according to embodiment C-240, wherein the compound of Formula (C-V-B1-iia-2) is a compound of Formula (C-V-B1-iia-2a) or Formula (C-V-B1-iia-2b):

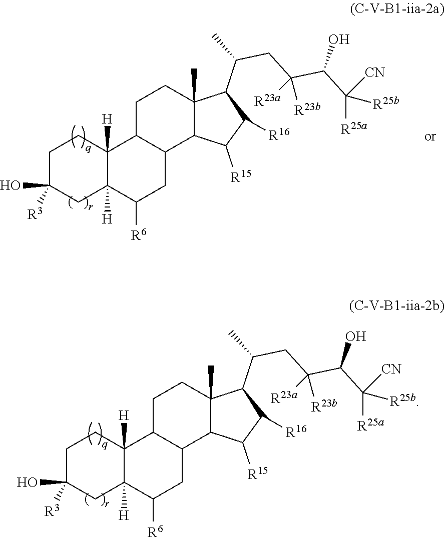

C-242. The compound according to embodiment C-168, wherein the compound of Formula (C-I) is a compound of Formula (C-VI):

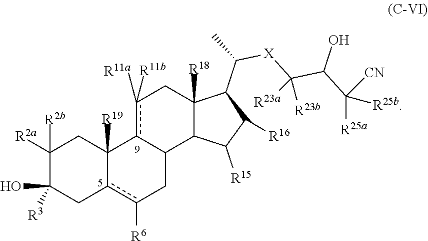

C-243. The compound according to embodiment C-242, wherein the compound of Formula (C-VI) is a compound of Formula (C-VIA) or Formula (C-VIB):

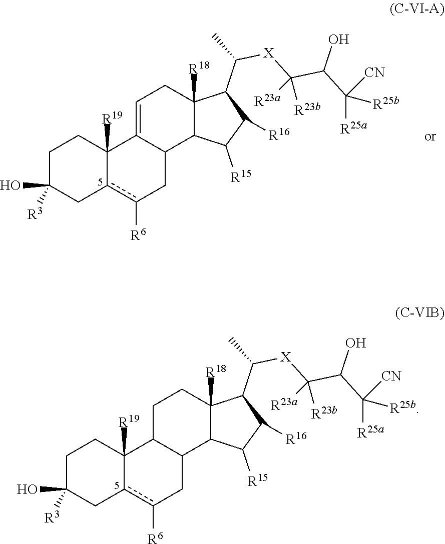

C-244. The compound according to embodiment C-243, wherein the compound of Formula (C-VI-A) is a compound of Formula (C-VI-A1):

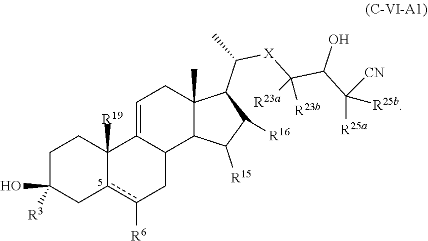

C-245. The compound according to embodiment C-244, wherein the compound of Formula (C-VI-A1) is a compound of Formula (C-VI-A1-i):

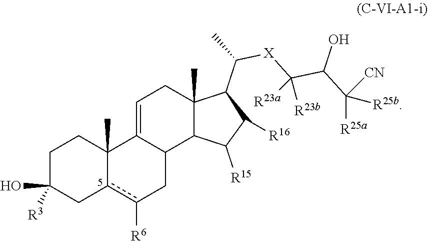

C-246. The compound according to embodiment C-245, wherein the compound of Formula (C-VI-A1-i) is a compound of Formula (C-VI-A1-ia):

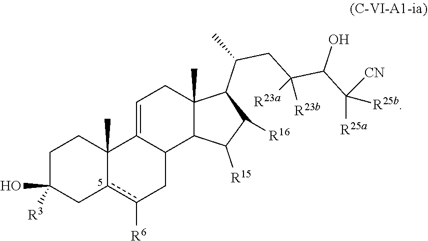

C-247. The compound according to embodiment C-246, wherein the compound of Formula (C-VI-A1-ia) is a compound of Formula (C-VI-A1-ia-1):

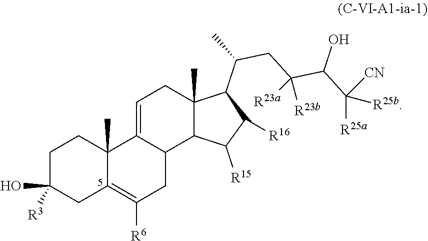

C-248. The compound according to embodiment C-247, wherein the compound of Formula (C-VI-A1-ia-1) is a compound of Formula (C-VI-A1-ia-1a) or Formula (C-VI-A1-ia-1b):

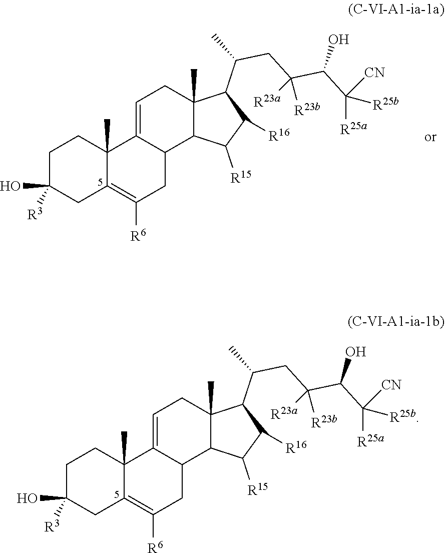

C-249. The compound according to embodiment C-246, wherein the compound of Formula (C-VI-A1-ia) is a compound of Formula (C-VI-A1-ia-2):

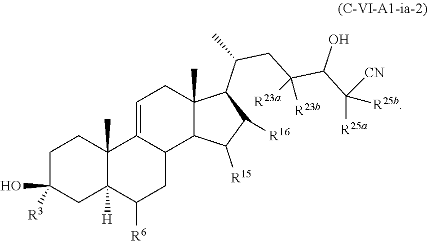

C-250. The compound according to embodiment C-249, wherein the compound of Formula (C-VI-A1-ia-2) is a compound of Formula (C-VI-A1-ia-2a) or Formula (C-VI-A1-ia-2b):

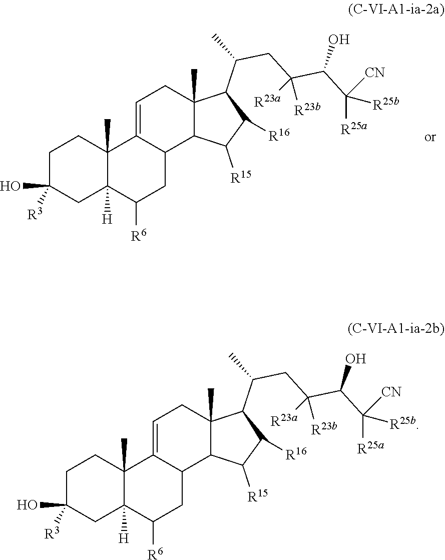

C-251. The compound according to embodiment C-244, wherein the compound of Formula (C-VI-A1) is a compound of Formula (C-VI-A1-ii):

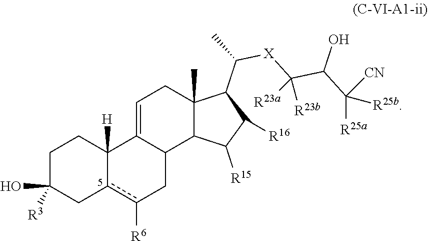

C-252. The compound according to embodiment C-251, wherein the compound of Formula (C-VI-A1-ii) is a compound of Formula (C-VI-A1-iia):

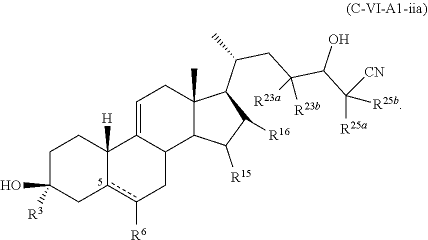

C-253. The compound according to embodiment C-252, wherein the compound of Formula (C-VI-A1-iia) is a compound of Formula (C-VI-A1-iia-1):

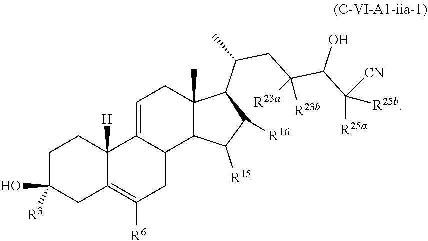

C-254. The compound according to embodiment C-253, wherein the compound of Formula (C-VI-A1-iia-1) is a compound of Formula (C-VI-A1-iia-1a) or Formula (C-VI-A1-iia-1b):

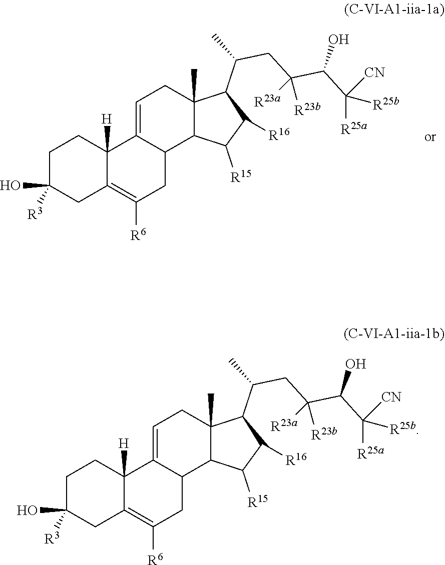

C-255. The compound according to embodiment C-252, wherein the compound of Formula (C-VI-A1-iia) is a compound of Formula (C-VI-A1-iia-2):

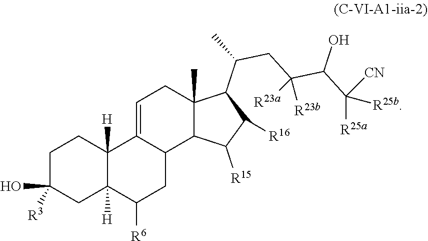

C-256. The compound according to embodiment C-255, wherein the compound of Formula (C-VI-A1-iia-2) is a compound of Formula (C-VI-A1-iia-2a) or Formula (C-VI-A1-iia-2b):

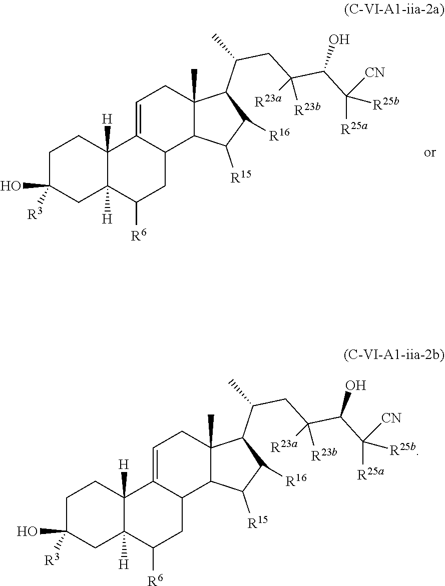

C-257. The compound according to embodiment C-243, wherein the compound of Formula (C-VI-B) is a compound of Formula (C-VI-B1):

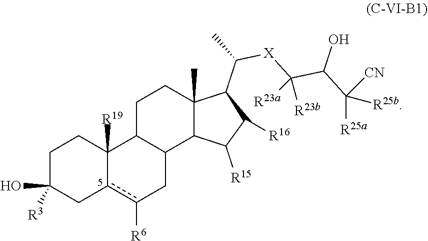

C-258. The compound according to embodiment C-257, wherein the compound of Formula (C-VI-B1) is a compound of Formula (C-VI-B1-i):

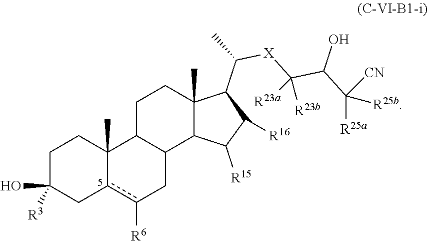

C-259. The compound according to embodiment C-258, wherein the compound of Formula (C-VI-B1-i) is a compound of Formula (C-VI-B1-ia):

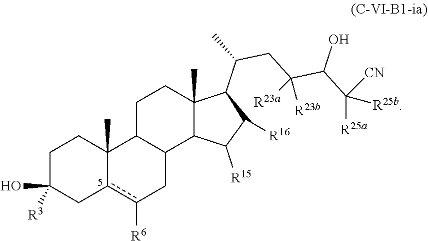

C-260. The compound according to embodiment C-259, wherein the compound of Formula (C-VI-B1-ia) is a compound of Formula (C-VI-B1-ia-1):

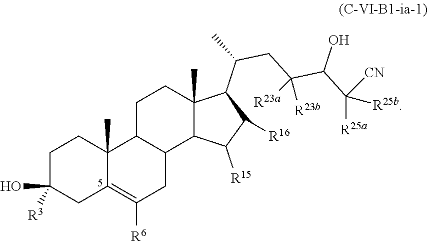

C-261. The compound according to embodiment C-260, wherein the compound of Formula (C-VI-B1-ia-1) is a compound of Formula (C-VI-B1-ia-1a) or

Formula (C-VI-B1-ia-1b):

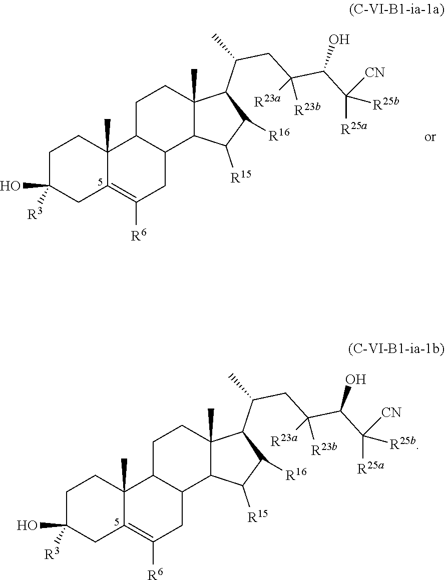

C-262. The compound according to embodiment C-259, wherein the compound of Formula (C-VI-B1-ia) is a compound of Formula (C-VI-B1-ia-2):

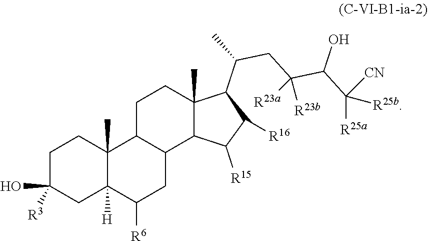

C-263. The compound according to embodiment C-262, wherein the compound of Formula (C-VI-B1-ia-2) is a compound of Formula (C-VI-B1-ia-2a) or Formula (C-VI-B1-ia-2b):

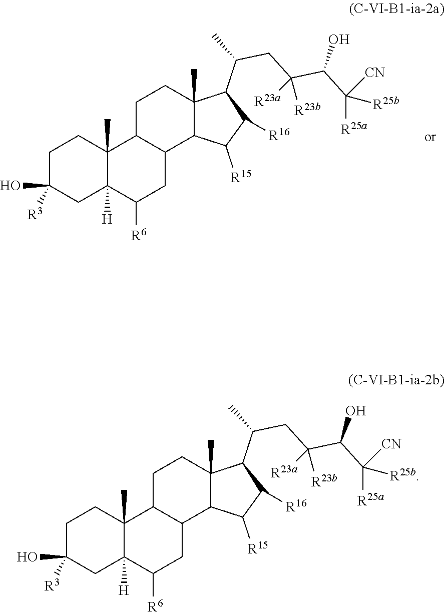

C-264. The compound according to embodiment C-257, wherein the compound of Formula (C-VI-B1) is a compound of Formula (C-VI-B1-ii):

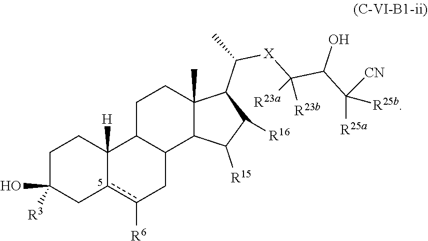

C-265. The compound according to embodiment C-264, wherein the compound of Formula (C-VI-B1-ii) is a compound of Formula (C-VI-B1-iia):

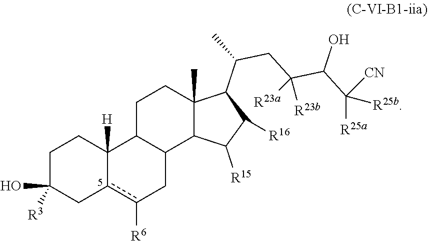

C-266. The compound according to embodiment C-265, wherein the compound of Formula (C-VI-B1-iia) is a compound of Formula (C-VI-B1-iia-1):

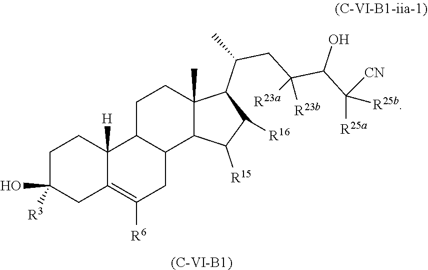

C-267. The compound according to embodiment C-266, wherein the compound of Formula (C-VI-B1-iia-1) is a compound of Formula (C-VI-B1-iia-1a) or Formula (C-VI-B1-iia-1b):

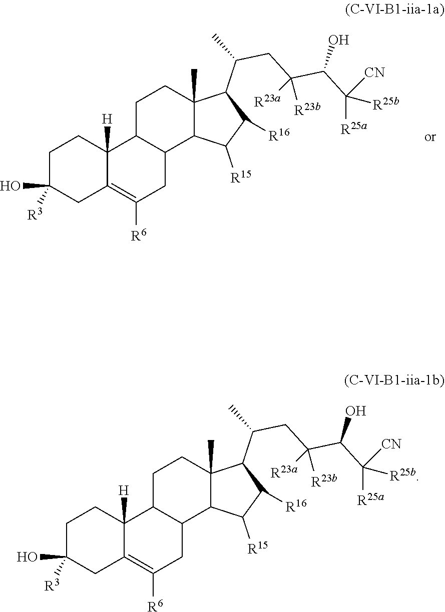

C-268. The compound according to embodiment C-265, wherein the compound of Formula (C-VI-B1-iia) is a compound of Formula (C-VI-B1-iia-2):

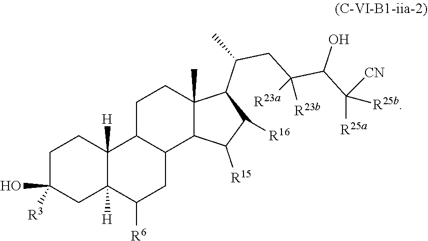

C-269. The compound according to embodiment C-268, wherein the compound of Formula (C-VI-B1-iia-2) is a compound of Formula (C-VI-B1-iia-2a) or Formula (C-VI-B1-iia-2b):

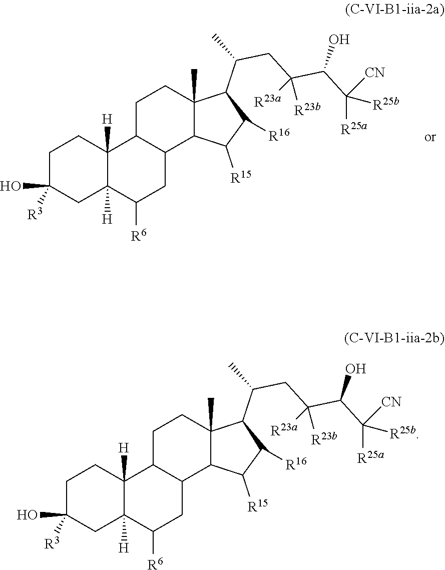

C-270. The compound according to any one of embodiments C-168, C-169, C-184, C-199, C-214, and C-242 wherein each of R^{2a} and R^{2b} is hydrogen.

C-271. The compound according to any one of embodiments C-168, C-169, C-184, C-199, C-214, and C-242, wherein each of R^{11a} and R^{11b} is hydrogen.

C-272. The compound according to any one of embodiments C-168 to C-269, wherein R³ is substituted or unsubstituted C₁₋₆alkyl.

C-273. The compound according to embodiment C-272, wherein R³ is —CH₃, —CH₂CH₃, —CH₂CH₂CH₃, or —CH₂OCH₃.

C-274. The compound according to embodiment C-273, wherein R³ is —CH₂CH₃.

C-275. The compound according to embodiment C-273, wherein R³ is —CH₂OCH₃.

C-276. The compound according to any one of embodiments C-168 to C-269, wherein R⁶ is hydrogen.

C-277. The compound according to any one of embodiments C-168 to C-269, wherein X is —(C(R^{X})₂—, wherein R^{X} is hydrogen or fluorine.

C-278. The compound according to embodiment C-277, wherein X is —CH₂—.

C-279. The compound according to any one of embodiments C-168 to C-269, wherein each of R^{23a} and R^{23b} is independently hydrogen.

C-280. The compound according to any one of embodiments C-168 to C-269, wherein each of R^{23a} and R^{23b} is independently fluorine.

C-281. The compound according to any one of embodiments C-168 to C-269, wherein R^{25a} is hydrogen or unsubstituted C₁₋₆alkyl.

C-282. The compound according to embodiment C-281, wherein R^{25a} is hydrogen, —CH₃, or —CH₂CH₃.

C-283. The compound according to embodiment C-282, wherein R^{25a} is hydrogen.

C-284. The compound according to embodiment C-282, wherein R^{25a} is —CH₃.

C-285. The compound according to embodiment C-282, wherein R^{25a} is —CH₂CH₃.

C-286. The compound according to any one of embodiments C-168 to C-269, wherein R^{25b} is hydrogen or unsubstituted C₁₋₆alkyl.

C-287. The compound according to embodiment C-286, wherein R 25b is hydrogen.

C-288. The compound according to embodiment C-286, wherein R^{25b} is —CH₃.

C-289. The compound according to any one of embodiments C-168 to C-269, wherein R^{25a} is unsubstituted C₁₋₆alkyl and R^{25b} is hydrogen or unsubstituted C₁₋₆alkyl.

C-290. The compound according to any one of embodiments C-168 to C-269, wherein R^{25a} is hydrogen or unsubstituted C₁₋₆alkyl and R^{25b} is hydrogen or unsubstituted C₁-6alkyl.

C-291. The compound according to embodiment C-290, wherein R^{25a} is —CH₃ and R^{25b} is —CH₃.

C-292. The compound according to embodiment C-290, wherein R^{25a} is —CH₂CH₃ and R^{25b} is hydrogen.

C-293. The compound according to embodiment C-290, wherein R^{25a} is hydrogen and R^{25b} is hydrogen.

C-294. The compound according to any one of embodiments C-168 to C-269, wherein R^{25a} and R^{25b} taken together with the carbon atom to which they are attached, form an unsubstituted C₃₋₆carbocyclyl.

C-295. The compound according to embodiment C-168 to C-269, wherein R^{25a} and R^{25b} taken together with the carbon atom to which they are attached, form a cyclopropyl.

C-296. The compound according to any one of embodiments C-168 to C-173, C-178 to C-179, C-184 to C-188, C-193 to C-194, C-199 to C-203, C-208 to C-209, C-214 to C-218, C-223 to C-224, C-229 to C-231, C-236 to C-237, C-242 to C-246, C-251 to C-252, C-257 to C-259, and C-264 to C-265, wherein the bond between C5 and C6 is a single bond and R⁵ is hydrogen in the alpha configuration.

C-297. The compound according to any one of embodiments C-168, C-214, and C-242, wherein the bond between C9 and C11 is a double bond, R⁹ is absent and R^{11a} is selected from the group consisting of hydrogen, halogen, substituted or unsubstituted C₁₋₆ alkyl, and —OR^{A11}, wherein R^{A11} is hydrogen or substituted or unsubstituted C₁₋₆alkyl, and R^{11b} is absent or hydrogen.

C-298. The compound according to any one of embodiments C-168, C-214, and C-242, wherein the bond between C9 and C11 is a single bond, R⁹ is hydrogen and R¹¹ is selected from the group consisting of halogen or substituted or unsubstituted C₁₋₆alkyl.

C-299. The compound according to any one of embodiments C-168 and C-214 to C-269, wherein each instance of R¹⁵ is independently hydrogen, substituted or unsubstituted C₁₋₆alkyl or substituted or unsubstituted C₃₋₆carbocyclyl.

C-300. The compound according to embodiment C-299, wherein each instance of R¹⁵ is independently hydrogen, unsubstituted C₁₋₆alkyl or unsubstituted C₃₋₆carbocyclyl.

C-301. The compound according to embodiment C-300, wherein each instance of R¹⁵ is independently hydrogen, —CH₃ or cyclopropyl.

C-302. The compound according to embodiment C-300, wherein each instance of R 15 is independently hydrogen.

C-303. The compound according to embodiment C-300, wherein each instance of R¹⁵ is independently —CH₃.

C-304. The compound according to embodiment C-300, wherein each instance of R¹⁵ is independently cyclopropyl.

C-305. The compound according to any one of embodiments C-168 and C-214 to C-269, wherein R¹⁶ is hydrogen, substituted or unsubstituted C₁₋₆alkyl or substituted or unsubstituted C₃₋₆carbocyclyl.

C-306. The compound according to embodiment C-305, wherein R¹⁶ is hydrogen or unsubstituted C₁₋₆alkyl.

C-307. The compound according to embodiment C-305, wherein R¹⁶ is hydrogen.

C-308. The compound according to embodiment C-305, wherein R¹⁶ is —CH₃.

C-309. The compound according to any one of embodiments C-168 and C-214 to C-269, wherein R¹⁵ and R¹⁶ are taken together to form a substituted or unsubstituted C₃₋₆ carbocyclyl.

C-310. The compound according to embodiment C-309, wherein R¹⁵ and R¹⁶ are taken together to form a cyclopropyl.

C-311. The compound according to any one of embodiments C-168 to C-170, C-184 to C-185, C-199 to C-200, C-214 to C-215 and C-243, wherein R¹⁸ is hydrogen, unsubstituted C₁₋₆alkyl or C₁₋₆alkyl substituted with OR^{A18}, wherein R^{A18} is substituted or unsubstituted C₁₋₆alkyl.

C-312. The compound according to embodiment C-311, wherein R¹⁸ is hydrogen, —CH₃, CH₂OCH₃, —OCH₃, —CH₂CH₃, or —CH(CH₃)₂.

C-313. The compound according to embodiment C-312, wherein R 18 is hydrogen, —CH₂CH₃ or —CH₃.

C-314. The compound according to embodiment C-313, wherein R¹⁸ is —CH₂CH₃.

C-315. The compound according to embodiment C-313, wherein R¹⁸ is —CH₃.

C-316. The compound according to embodiment C-313, wherein R¹⁸ is hydrogen.

C-317. The compound according to any one of embodiments C-168 to C-171, C-184 to C-186, C-199 to C-201, C-214 to C-216, C-229, and C-242 to C-244, wherein R¹⁹ is hydrogen, unsubstituted C₁₋₆alkyl or C₁₋₆alkyl substituted with OR^{A19}, wherein R^{A19} is substituted or unsubstituted C₁₋₆alkyl.

C-318. The compound according to embodiment C-317, wherein R¹⁹ is hydrogen, —CH₃, CH₂OCH₃, —OCH₃, —CH₂CH₃, or —CH(CH₃)₂.

C-319. The compound according to embodiment C-318, wherein R¹⁹ is hydrogen or —CH₃.

C-320. The compound according to embodiment C-319, wherein R¹⁹ is —CH₃.

C-321. The compound according to embodiment C-319, wherein R¹⁹ is hydrogen.

C-322. The compound according to embodiment C-168, wherein the compound of Formula (C-I) is selected from the group consisting of Compounds C1-C35.

C-323. The compound according to embodiment C-168, wherein the compound of Formula (C-I) is selected from the group consisting of Compounds C1-C17.

C-324. The compound according to embodiment 168, wherein the compound of Formula (C-I) is selected from the group consisting of C-1, C-2, C-3, C-5, C-7, C-8, C-9, and C-11.

C-325. A pharmaceutical composition comprising a compound according to any one of embodiments C-168 to C-324, and a pharmaceutically acceptable carrier.

C-326. A method for treating a CNS-related condition in a subject, comprising administering to the subject an effective amount of a compound according to any one of embodiments C-168 to C-324, or a pharmaceutical composition according to embodiment C-325.

C-327. The method according to embodiment C-326, wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease) and tinnitus.

C-328. A method of inducing sedation or anesthesia in a subject, comprising administering to the subject an effective amount of a compound according to any one of embodiments C-168 to C-324, or a pharmaceutical composition according to embodiment C-325.

C-329. A compound according to any one of embodiments C-168 to C-324, or a pharmaceutical composition according to embodiment C-325, for use in treating a CNS-related condition in a subject.

C-330. The compound, or pharmaceutical composition for use according to embodiment C-329, wherein the wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease) and tinnitus.

C-331. A compound according to any one of embodiments C-168 to C-324, or a pharmaceutical composition according to embodiment C-325, for use inducing sedation or anesthesia in a subject.

C-332. Use of a compound according to any one of embodiments C-168 to C-324, or a pharmaceutical composition according to embodiment C-325, for the manufacture of a medicament for treating a CNS-related condition in a subject.

C-333. The use according to embodiment C-332, wherein the wherein the wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease) and tinnitus.

C-334. Use of a compound according to any one of embodiments C-168 to C-324, or a pharmaceutical composition according to embodiment C-325, for the manufacture of a medicament for inducing sedation or anesthesia in a subject.

D-1. A compound of Formula (D-I):

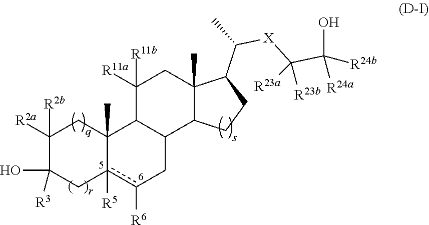

or a pharmaceutically acceptable salt thereof;
wherein:
   each of R^{2a} and R^{2b} is independently hydrogen, halogen, substituted or unsubstituted C₁₋₆alkyl, substituted or unsubstituted C₃₋₁₀carbocyclyl, or —OR^{A2} wherein R^{A2} is hydrogen or substituted or unsubstituted C₁₋₆alkyl;
   R³ is substituted or unsubstituted C₁₋₆alkyl, substituted or unsubstituted C₂₋₆ alkenyl, or substituted or unsubstituted C₂₋₆alkynyl, or substituted or unsubstituted C₃₋₁₀ carbocyclyl;
   R⁵ is absent, hydrogen, halogen, or substituted or unsubstituted C₁₋₆alkyl;
   R⁶ is hydrogen, substituted or unsubstituted C₁₋₆alkyl, or halogen;
   R^{11a} is hydrogen or —OR^{A11}, wherein R^{A11} is hydrogen or substituted or unsubstituted C₁₋₆alkyl, and R^{11b} is hydrogen; or R^{11a} and R^{11b} are joined to form an oxo (═O) group;
   X is —(C(R^{X})₂)ₙ— or —O—, wherein R^{X} is hydrogen or fluorine, or one R^{X} group and R^{23b} are joined to form a double bond;
   n is 1, 2 or 3;
   each of R^{23a} and R^{23b} is independently hydrogen or fluorine;
   R^{24a} is selected from the group consisting of substituted or unsubstituted C₁₋₆alkyl, substituted or unsubstituted C₂₋₆alkenyl, substituted or unsubstituted C₂₋₆ alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl group; and
   R^{24b} is hydrogen or a substituted or unsubstituted C₁₋₆alkyl;
   or R^{24a} and R^{24b}, taken together with the carbon atom to which they are attached, form a substituted or unsubstituted C₃₋₆carbocyclyl, or substituted or unsubstituted 3- to 10-membered heterocyclyl;
   q is 0, 1 or 2;
   r is 1 or 2;
   s is 1, 2, 3 or 4;
   provided that a) q and r are not both 1, or b) when q is 0, r is not 2; and
   [symbol] represents a single or double bond; provided if the bond between C5 and C6 is a double bond, R⁵ is absent.

D-2. The compound or a pharmaceutically acceptable salt thereof according to embodiment D-1, wherein the compound of Formula (D-I) is a compound of Formula (D-Ia) or (D-Ib):

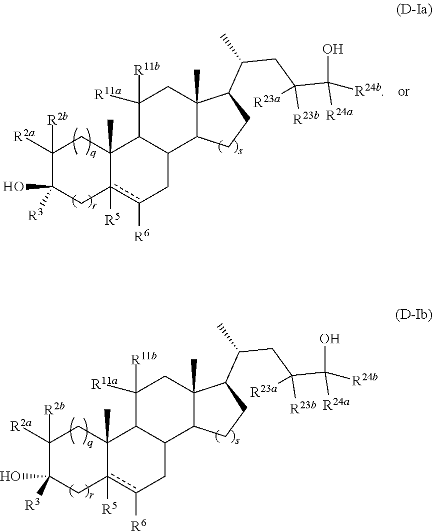

D-3. The compound or a pharmaceutically acceptable salt thereof according to embodiment D-2, wherein the compound of Formula (D-Ia) is a compound of Formula (D-Ia-1):

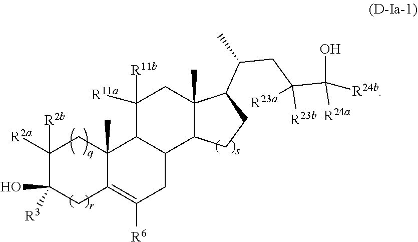

D-4. The compound or a pharmaceutically acceptable salt thereof according to embodiment D-3, wherein the compound of Formula (D-Ia-1) is a compound of Formula (D-Ia-1i):

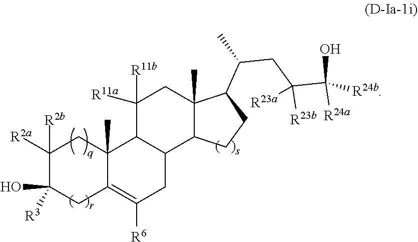

D-5. The compound or a pharmaceutically acceptable salt thereof according to embodiment D-3, wherein the compound of Formula (D-Ia-1) is a compound of Formula (D-Ia-1ii):

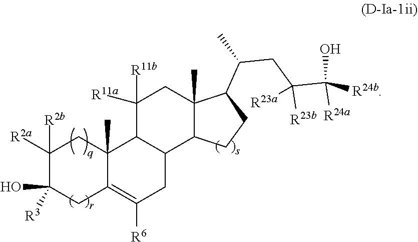

D-6. The compound or a pharmaceutically acceptable salt thereof according to embodiment D-2, wherein the compound of Formula (D-Ia) is a compound of Formula (D-Ia-2):

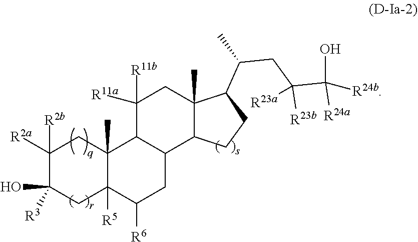

D-7. The compound or a pharmaceutically acceptable salt thereof according to embodiment D-6, wherein the compound of Formula (D-Ia-2) is a compound of Formula (D-Ia-2i):

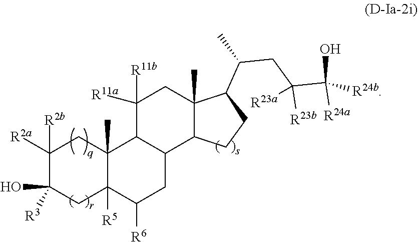

D-8. The compound or a pharmaceutically acceptable salt thereof according to embodiment D-6, wherein the compound of Formula (D-Ia-2) is a compound of Formula (D-Ia-2ii):

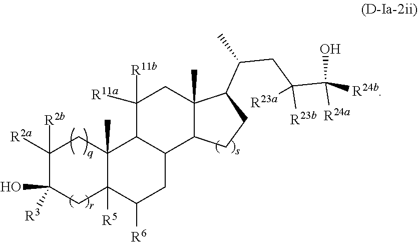

D-9. The compound or a pharmaceutically acceptable salt thereof according to embodiment D-2, wherein the compound of Formula (D-Ib) is a compound of Formula (D-Ib-1):

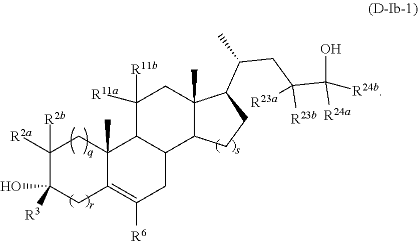

D-10. The compound or a pharmaceutically acceptable salt thereof according to embodiment D-9, wherein the compound of Formula (D-Ib-1) is a compound of Formula (D-Ib-1i):

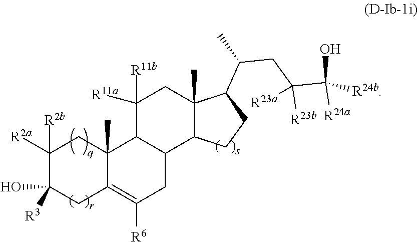

D-11. The compound or a pharmaceutically acceptable salt thereof according to embodiment D-9, wherein the compound of Formula (D-Ib-1) is a compound of Formula (D-Ib-1ii):

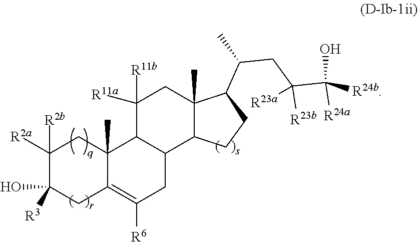

D-12. The compound or a pharmaceutically acceptable salt thereof according to embodiment D-2, wherein the compound of Formula (D-Ib) is a compound of Formula (D-Ib-2):

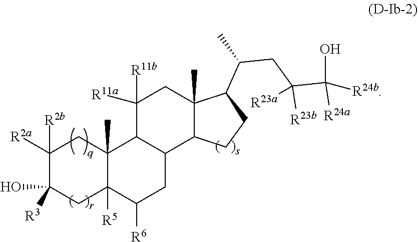

D-13. The compound or a pharmaceutically acceptable salt thereof according to embodiment D-12, wherein the compound of Formula (D-Ib-2) is a compound of Formula (D-Ib-2i):

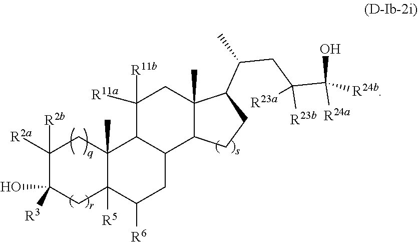

D-14. The compound or a pharmaceutically acceptable salt thereof according to embodiment D-12, wherein the compound of Formula (D-Ib-2) is a compound of Formula (D-Ib-2ii):

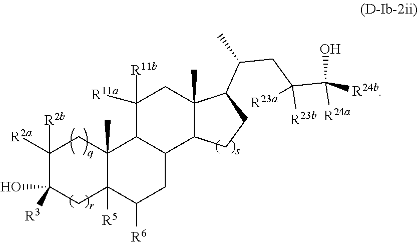

D-15. The compound or a pharmaceutically acceptable salt thereof according to embodiment D-1, wherein the compound of Formula (D-I) is a compound of Formula (D-II):

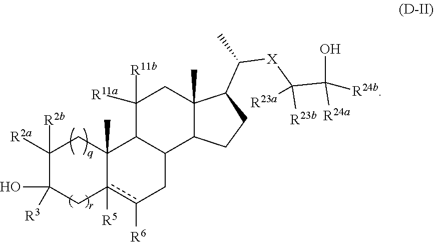

D-16. The compound or a pharmaceutically acceptable salt thereof according to embodiment D-15, wherein the compound of Formula (D-II) is a compound of Formula (D-IIa) or (D-IIb):

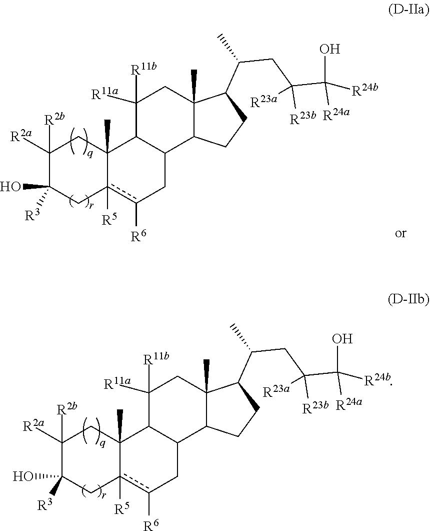

D-17. The compound or a pharmaceutically acceptable salt thereof according to embodiment D-16, wherein the compound of Formula (D-IIa) is a compound of Formula (D-IIa-1):

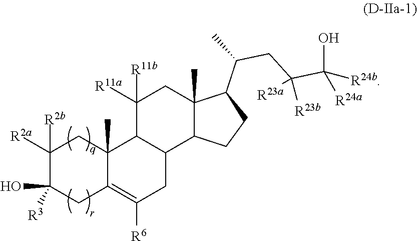

D-18. The compound or a pharmaceutically acceptable salt thereof according to embodiment D-17, wherein the compound of Formula (D-IIa-1) is a compound of Formula (D-IIa-1i):

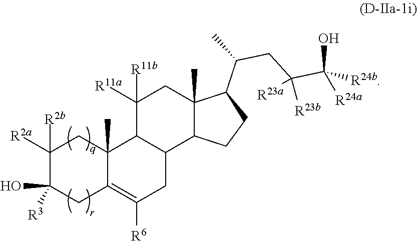

D-19. The compound or a pharmaceutically acceptable salt thereof according to embodiment D-17, wherein the compound of Formula (D-IIa-1) is a compound of Formula (D-IIa-1ii):

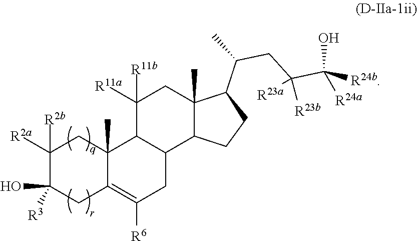

D-20. The compound or a pharmaceutically acceptable salt thereof according to embodiment D-16, wherein the compound of Formula (D-IIa) is a compound of Formula (D-IIa-2):

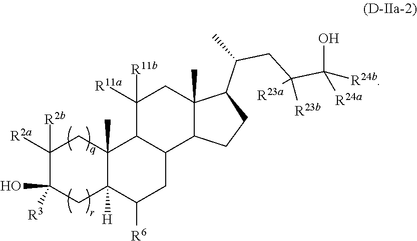

D-21. The compound or a pharmaceutically acceptable salt thereof according to embodiment D-20, wherein the compound of Formula (D-IIa-2) is a compound of Formula (D-IIa-2i):

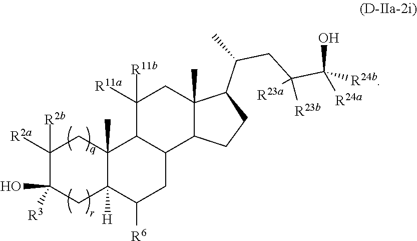

D-22. The compound or a pharmaceutically acceptable salt thereof according to embodiment D-20, wherein the compound of Formula (D-IIa-2) is a compound of Formula (D-IIa-2ii):

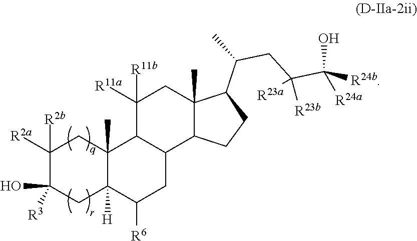

D-23. The compound or a pharmaceutically acceptable salt thereof according to embodiment D-16, wherein the compound of Formula (D-IIb) is a compound of Formula (D-IIb-1):

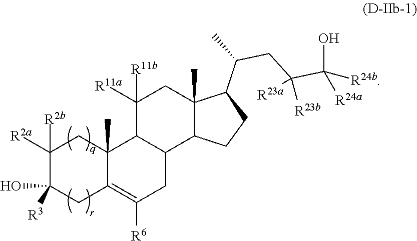

D-24. The compound or a pharmaceutically acceptable salt thereof according to embodiment D-23, wherein the compound of Formula (D-IIb-1) is a compound of Formula (D-IIb-1i):

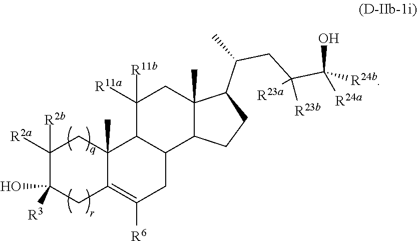

D-25. The compound or a pharmaceutically acceptable salt thereof according to embodiment D-23, wherein the compound of Formula (D-IIb-1) is a compound of Formula (D-IIb-1ii):

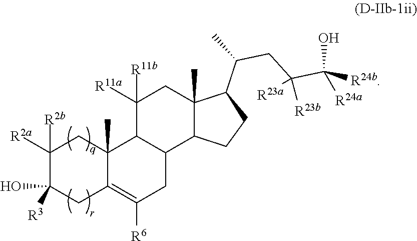

D-26. The compound or a pharmaceutically acceptable salt thereof according to embodiment D-16, wherein the compound of Formula (D-IIb) is a compound of Formula (D-IIb-2):

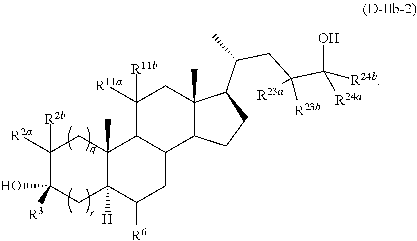

D-27. The compound or a pharmaceutically acceptable salt thereof according to embodiment D-26, wherein the compound of Formula (D-IIb-2) is a compound of Formula (D-IIb-2i):

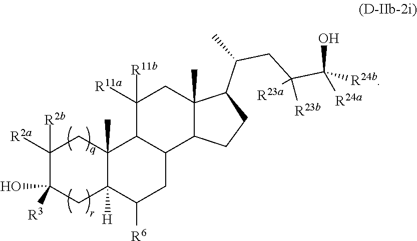

D-28. The compound or a pharmaceutically acceptable salt thereof according to embodiment D-26, wherein the compound of Formula (D-IIb-2) is a compound of Formula (D-IIb-2ii):

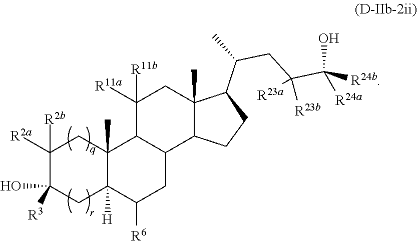

D-29. The compound or a pharmaceutically acceptable salt thereof according to embodiment D-1, wherein the compound of Formula (D-I) is a compound of Formula (D-III):

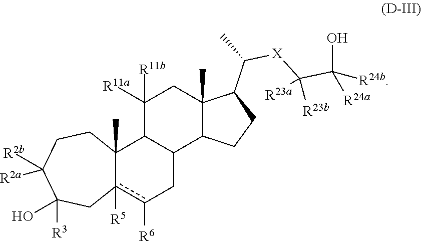

D-30. The compound or a pharmaceutically acceptable salt thereof according to embodiment D-29, wherein the compound of Formula (D-III) is a compound of Formula (D-IIIa) or Formula (D-IIIb):

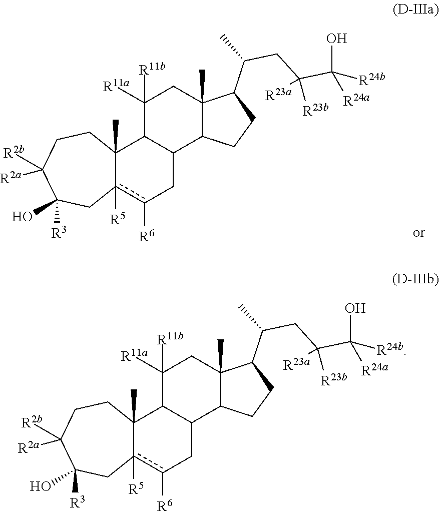

D-31. The compound or a pharmaceutically acceptable salt thereof according to embodiment D-30, wherein the compound of Formula (D-IIIa) is a compound of Formula (D-IIIa-1):

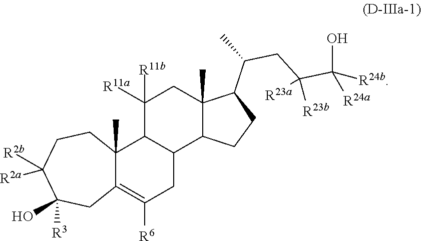

D-32. The compound or a pharmaceutically acceptable salt thereof according to embodiment D-31, wherein the compound of Formula (D-IIIa-1) is a compound of Formula (D-IIIa-1i):

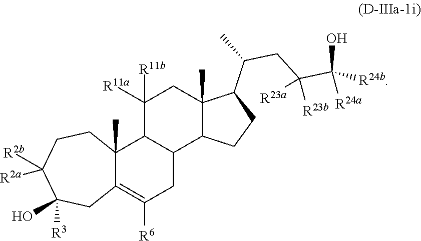

D-33. The compound or a pharmaceutically acceptable salt thereof according to embodiment D-31, wherein the compound of Formula (D-IIIa-1) is a compound of Formula (D-IIIa-1ii):

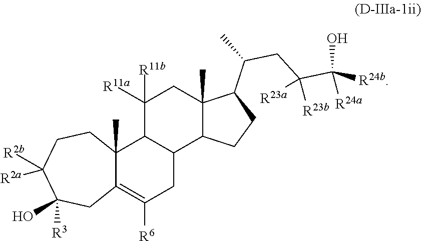

D-34. The compound or a pharmaceutically acceptable salt thereof according to embodiment D-30, wherein the compound of Formula (D-IIIa) is a compound of Formula (D-IIIa-2):

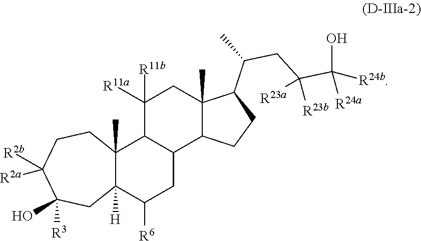

D-35. The compound or a pharmaceutically acceptable salt thereof according to embodiment D-34, wherein the compound of Formula (D-IIIa-2) is a compound of Formula (D-IIIa-2i):

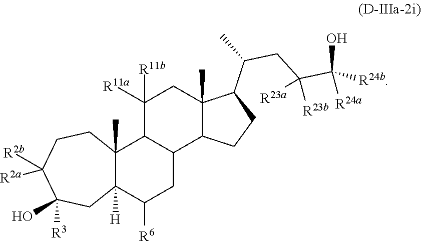

D-36. The compound or a pharmaceutically acceptable salt thereof according to embodiment D-34, wherein the compound of Formula (D-IIIa-2) is a compound of Formula (D-IIIa-2ii):

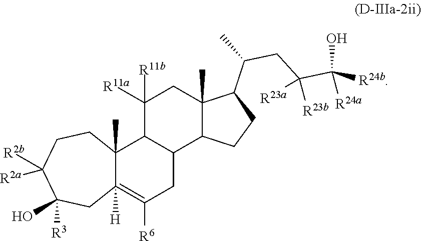

D-37. The compound or a pharmaceutically acceptable salt thereof according to embodiment D-30, wherein the compound of Formula (D-IIIb) is a compound of Formula (D-IIIb-1):

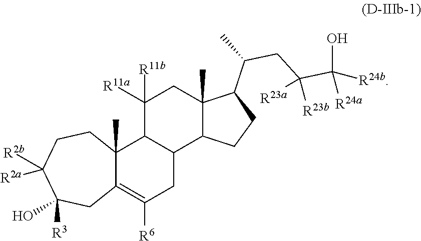

D-38. The compound or a pharmaceutically acceptable salt thereof according to embodiment D-37, wherein the compound of Formula (D-IIIb-1) is a compound of Formula (D-IIIb-1i):

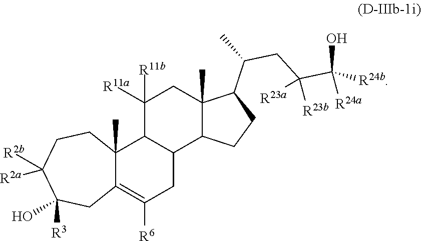

D-39. The compound or a pharmaceutically acceptable salt thereof according to embodiment D-37, wherein the compound of Formula (D-IIIb-1) is a compound of Formula (D-IIIb-1ii):

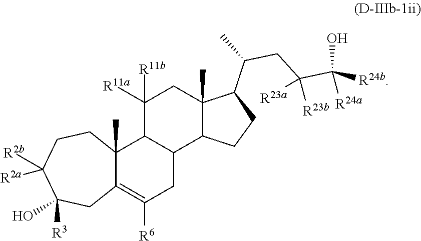

D-40. The compound or a pharmaceutically acceptable salt thereof according to embodiment D-30, wherein the compound of Formula (D-IIIb) is a compound of Formula (D-IIIb-2):

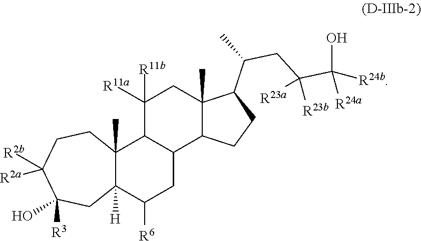

D-41. The compound or a pharmaceutically acceptable salt thereof according to embodiment D-40, wherein the compound of Formula (D-IIIb-2) is a compound of Formula (D-IIIb-2i):

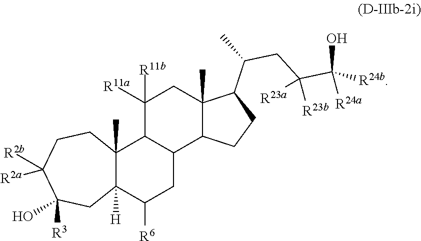

D-42. The compound or a pharmaceutically acceptable salt thereof according to embodiment D-40, wherein the compound of Formula (D-IIIb-2) is a compound of Formula (D-IIIb-2ii):

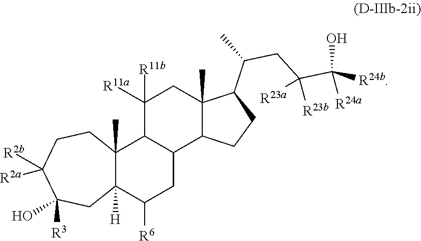

D-43. The compound or a pharmaceutically acceptable salt thereof according to embodiment D-1, wherein the compound of Formula (D-I) is a compound of Formula (D-IV):

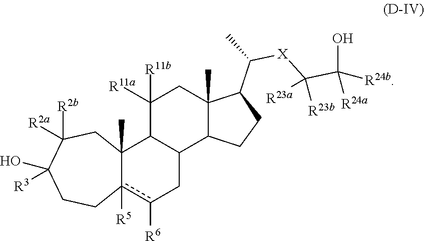

D-44. The compound or a pharmaceutically acceptable salt thereof according to embodiment D-43, wherein the compound of Formula (D-IV) is a compound of Formula (D-IVa) or Formula (D-IVb):

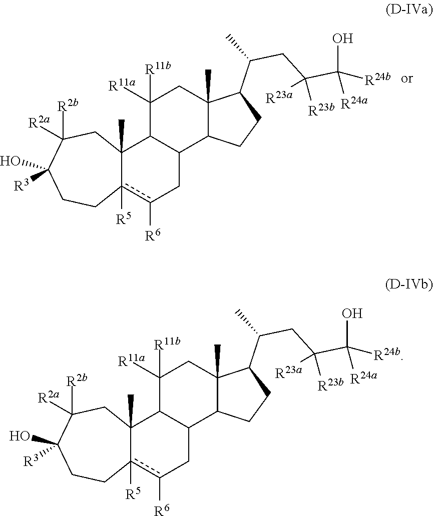

D-45. The compound or a pharmaceutically acceptable salt thereof according to embodiment D-44, wherein the compound of Formula (D-IVa) is a compound of Formula (D-IVa-1):

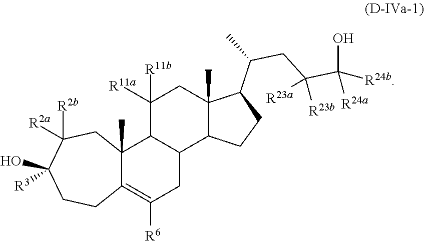

D-46. The compound or a pharmaceutically acceptable salt thereof according to embodiment D-45, wherein the compound of Formula (D-IVa-1) is a compound of Formula (D-IVa-1i):

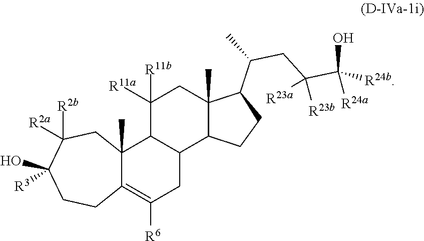

D-47. The compound or a pharmaceutically acceptable salt thereof according to embodiment D-45, wherein the compound of Formula (D-IVa-1) is a compound of Formula (D-IVa-1ii):

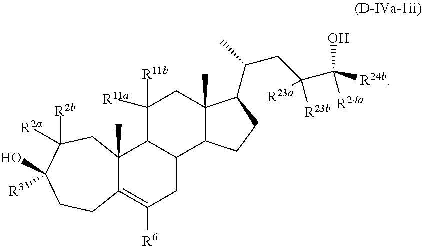

D-48. The compound or a pharmaceutically acceptable salt thereof according to embodiment D-44, wherein the compound of Formula (D-IVa) is a compound of Formula (D-IVa-2):

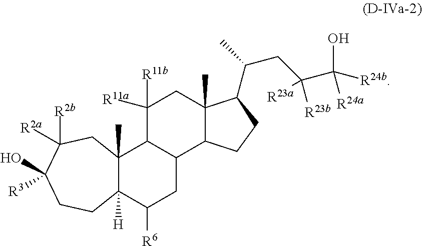

D-49. The compound or a pharmaceutically acceptable salt thereof according to embodiment D-48, wherein the compound of Formula (D-IVa-2) is a compound of Formula (D-IVa-2i):

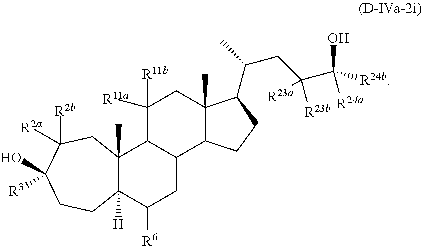

D-50. The compound or a pharmaceutically acceptable salt thereof according to embodiment D-48, wherein the compound of Formula (D-IVa-2) is a compound of Formula (D-IVa-2ii):

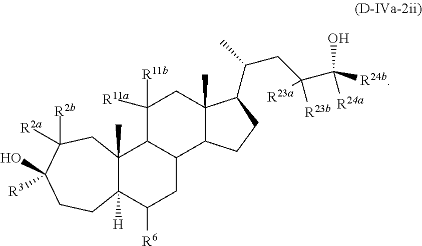

D-51. The compound or a pharmaceutically acceptable salt thereof according to embodiment D-44, wherein the compound of Formula (D-IVb) is a compound of Formula (D-IVb-1):

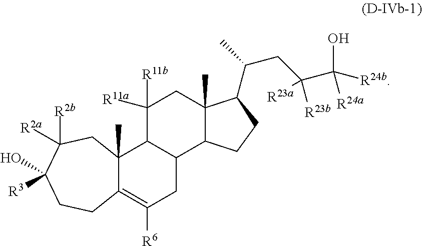

D-52. The compound or a pharmaceutically acceptable salt thereof according to embodiment D-51, wherein the compound of Formula (D-IVb-1) is a compound of Formula (D-IVb-1i):

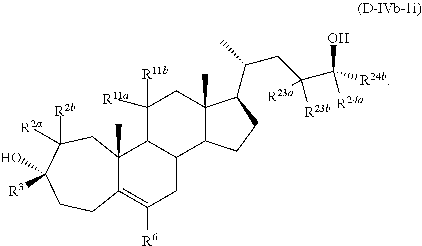

D-53. The compound or a pharmaceutically acceptable salt thereof according to embodiment D-51, wherein the compound of Formula (D-IVb-1) is a compound of Formula (D-IVb-1ii):

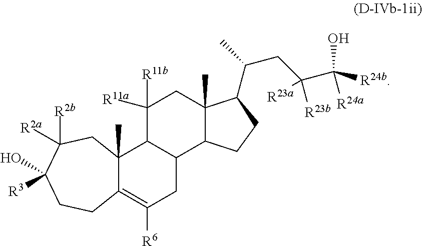

D-54. The compound or a pharmaceutically acceptable salt thereof according to embodiment D-44, wherein the compound of Formula (D-IVb) is a compound of Formula (D-IVb-2):

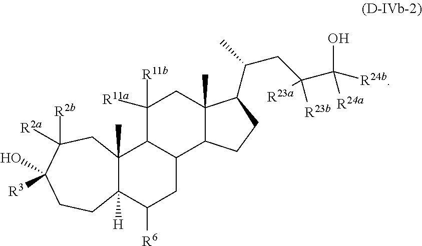

D-55. The compound or a pharmaceutically acceptable salt thereof according to embodiment D-54, wherein the compound of Formula (D-IVb-2) is a compound of Formula (D-IVb-2i):

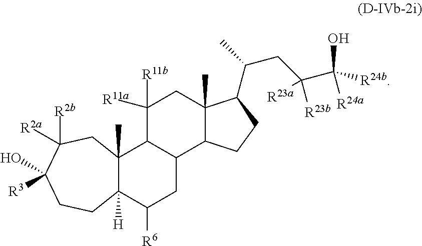

D-56. The compound or a pharmaceutically acceptable salt thereof according to embodiment D-54, wherein the compound of Formula (D-IVb-2) is a compound of Formula (D-IVb-2ii):

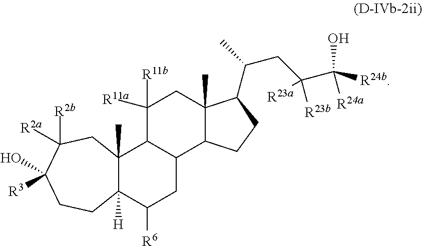

D-57. The compound or pharmaceutically acceptable salt thereof according to any one of embodiments D-1 to D-56, wherein each of R^{2a} and R^{2b} is hydrogen.

D-58. The compound or pharmaceutically acceptable salt thereof according to any one of embodiments D-1 to D-56, wherein R³ is unsubstituted C₁₋₆alkyl.

D-59. The compound or pharmaceutically acceptable salt thereof according to embodiment D-58, wherein R³ is —CH₃, —CH₂CH₃, or —CH₂CH₂CH₃.

D-60. The compound or pharmaceutically acceptable salt thereof according to embodiment D-59, wherein R³ is —CH₃.

D-61. The compound or pharmaceutically acceptable salt thereof according to embodiment D-59 wherein R³ is —CH₂CH₃.

D-62. The compound or pharmaceutically acceptable salt thereof according to any one of embodiments D-1 to D-56, wherein each of R^{11a} and R^{11b} is hydrogen.

D-63. The compound or pharmaceutically acceptable salt thereof according to embodiments D-1 to D-2, D-6 to D-8, D-12 to D-16, D-29 to D-30, and D-43 to D-44, wherein R⁵, when present, is hydrogen.

D-64. The compound or pharmaceutically acceptable salt thereof according to embodiments D-1 to D-2, D-6 to D-8, D-12 to D-16, D-29 to D-30, and D-43 to D-44, wherein R⁵, when present, is in the alpha configuration.

D-65. The compound or pharmaceutically acceptable salt thereof according to embodiments D-1 to D-56, wherein R⁶ is hydrogen.

D-66. The compound or pharmaceutically acceptable salt thereof according to any one of embodiments D-1 to D-56, wherein each of R^{23a} and R^{23b} is independently hydrogen.

D-67. The compound or pharmaceutically acceptable salt thereof according to any one of embodiments D-1 to D-56, wherein at least one of R^{23a} and R^{23b} is fluorine.

D-68. The compound or pharmaceutically acceptable salt thereof according to any one of embodiments D-1 to D-56, wherein each of R^{23a} and R^{23b} is fluorine.

D-69. The compound or pharmaceutically acceptable salt thereof according to any one of embodiments D-1 to D-56, wherein R^{24a} is substituted C₁₋₆alkyl.

D-70. The compound or pharmaceutically acceptable salt thereof according to embodiment D-69, wherein R^{24a} is C₁₋₆alkyl substituted with fluorine.

D-71. The compound or pharmaceutically acceptable salt thereof according to embodiment D-70, wherein R^{24a} is —CF₃.

D-72. The compound or pharmaceutically acceptable salt thereof according to any one of embodiments D-1 to D-56, wherein R^{24a} is unsubstituted C₁₋₆alkyl.

D-73. The compound or pharmaceutically acceptable salt thereof according to embodiment D-72, wherein R^{24a} is unsubstituted-CH₃.

D-74. The compound or pharmaceutically acceptable salt thereof according to any one of embodiments D-1 to D-56, wherein R^{24b} is unsubstituted C₁₋₆alkyl.

D-75. The compound or pharmaceutically acceptable salt thereof according to any embodiment D-74, wherein R^{24b} is —CH₃.

D-76. The compound or pharmaceutically acceptable salt thereof according to any one of embodiments D-1 to D-56, wherein R^{24b} is substituted C₁₋₆alkyl.

D-77. The compound or pharmaceutically acceptable salt thereof according to embodiment D-76, wherein R^{24b} is —CF₃.

D-78. The compound or pharmaceutically acceptable salt thereof according to any one of embodiments D-1 to D-56, wherein R^{24a} is —CF₃ and R^{24b} is —CH₃.

D-79. The compound or pharmaceutically acceptable salt thereof according to any one of embodiments D-1 to D-56, wherein R^{24a} is —CH₃ and R^{24b} is —CH₃.

D-80. The compound or pharmaceutically acceptable salt thereof according to any one of embodiments D-1 to D-56, wherein R^{24a} is —CH₃ and R^{24b} is —CF₃.

D-81. The compound or pharmaceutically acceptable salt thereof according to any one of embodiments D-1 to D-56, wherein q is 0 and r is 1.

D-82. The compound or pharmaceutically acceptable salt thereof according to any one of embodiments D-1 to D-28, wherein q is 1 and r is 2.

D-83. The compound or pharmaceutically acceptable salt thereof according to any one of embodiments D-1 to D-28, wherein q is 2 and r is 1.

D-84. The compound or pharmaceutically acceptable salt thereof according to any one of embodiments D-1 to D-28, wherein q is 2 and r is 2.

D-85. The compound or pharmaceutically acceptable salt thereof according to any one of embodiments D-1 to D-14, wherein s is 1.

D-86. The compound or pharmaceutically acceptable salt thereof according to any one of embodiments D-1 to D-14, wherein s is 2.

D-87. The compound or pharmaceutically acceptable salt thereof according to any one of embodiments D-1 to D-14, wherein s is 3.

D-88. The compound or pharmaceutically acceptable salt thereof according to any one of embodiments D-1 to D-14, wherein s is 4.

D-89. The compound or pharmaceutically acceptable salt thereof according to embodiment D-1, wherein the compound of Formula (D-I) is selected from the group consisting of D-1, D-2, D-3, D-4, D-5, D-6, D-7, D-8, D-9, D-9a, D-10, D-11, D-12, D-13, D-14, D-15, D-16, D-17 and pharmaceutically acceptable salts thereof.

D-90. The compound or pharmaceutically acceptable salt thereof according to embodiment D-1, wherein the compound of Formula (D-I) is selected from the group consisting of D-1, D-2, D-3, D-4, D-5, D-6, D-7, D-8, D-9, D-10, D-11, D-12, D-13, D-14, D-15, D-16, D-17 and pharmaceutically acceptable salts thereof.

D-91. A pharmaceutical composition comprising a compound or pharmaceutically acceptable salt thereof according to any one of embodiments D-1 to D-90, and a pharmaceutically acceptable carrier.

D-92. A method for treating a CNS-related condition in a subject comprising administering to the subject an effective amount of a compound or pharmaceutically acceptable salt thereof according to any one of embodiments D-1 to D-90, or a pharmaceutical composition according to embodiment D-91.

D-93. The method according to embodiment D-92, wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease) and tinnitus.

D-94. A method of inducing sedation or anesthesia in a subject, comprising administering to the subject an effective amount of a compound or pharmaceutically acceptable salt thereof according to any one of embodiments D-1 to D-90, or a pharmaceutical composition according to embodiment D-91.

D-95. A compound or pharmaceutically acceptable salt thereof according to any one of embodiments D-1 to D-90, or a pharmaceutical composition according to embodiment D-91, for use in treating a CNS-related condition in a subject.

D-96. The compound or pharmaceutically acceptable salt thereof, or pharmaceutical composition for use according to embodiment D-95, wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease) and tinnitus.

D-97. A compound or pharmaceutically acceptable salt thereof according to any one of embodiments D-1 to D-90, or a pharmaceutical composition according to embodiment D-91, for use inducing sedation or anesthesia in a subject.

D-98. Use of a compound or pharmaceutically acceptable salt thereof according to any one of embodiments D-1 to D-90, or a pharmaceutical composition according to embodiment D-91, for the manufacture of a medicament for treating a CNS-related condition in a subject.

D-99. The use according to embodiment D-98, wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease) and tinnitus.

D-100. Use of a compound or pharmaceutically acceptable salt thereof according to any one of embodiments D-1 to D-90, or a pharmaceutical composition according to embodiment D-91, for the manufacture of a medicament for inducing sedation or anesthesia in a subject.

D-101. A compound of Formula (D-I):

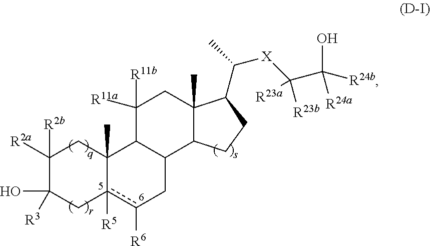

wherein:
   each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein R^{A2} is hydrogen or substituted or unsubstituted C₁₋₆alkyl;
   R³ is substituted or unsubstituted C₁₋₆alkyl, substituted or unsubstituted C₂₋₆ alkenyl, or substituted or unsubstituted C₂₋₆alkynyl, or substituted or unsubstituted C₃₋₁₀carbocyclyl;
   R⁵ is absent, hydrogen, halogen, or substituted or unsubstituted C₁₋₆alkyl;
   R⁶ is hydrogen, substituted or unsubstituted C₁₋₆alkyl, or halogen;
   R^{11a} is hydrogen or —OR^{A11}, wherein R^{A11} is hydrogen or substituted or unsubstituted C₁₋₆alkyl, and R^{11b} is hydrogen; or R^{11a} and R^{11b} are joined to form an oxo (═O) group;
   X is —(C(R^{X})₂)ₙ— or —O—, wherein R^{X} is hydrogen or fluorine, or one R^{X} group and R^{23b} are joined to form a double bond;
   n is 1, 2 or 3;
   each of R^{23a} and R^{23b} is independently hydrogen or fluorine;
   R^{24a} is selected from the group consisting of substituted or unsubstituted C₁₋₆alkyl, substituted or unsubstituted C₂₋₆alkenyl, substituted or unsubstituted C₂₋₆ alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl group; and
   R^{24b} is hydrogen or a substituted or unsubstituted C₁₋₆alkyl;
   or R^{24a} and R^{24b}, taken together with the carbon atom to which they are attached, form a substituted or unsubstituted C₃₋₆carbocyclyl, or substituted or unsubstituted 3- to 10-membered heterocyclyl;
   q is 0, 1 or 2;
   r is 1 or 2;
   s is 1 or 2;
   provided that a) q and r are not both 1, or b) when q is 0, r is not 2; and
   [symbol] represents a single or double bond;
provided if the bond between C5 and C6 is a double bond, R⁵ is absent.

D-102. The compound according to embodiment 101, wherein the compound of Formula (D-I) is a compound of Formula (D-Ia) or (D-Ib):

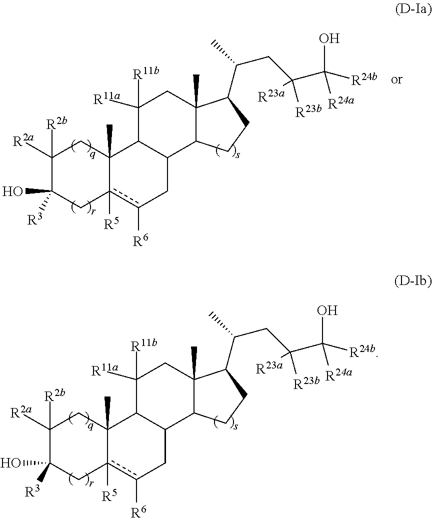

D-103. The compound according to embodiment D-102, wherein the compound of Formula (D-Ia) is a compound of Formula (D-Ia-1):

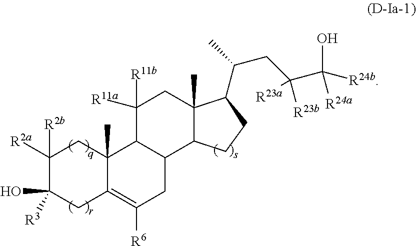

D-104. The compound according to embodiment D-103, wherein the compound of Formula (D-Ia-1) is a compound of Formula (D-Ia-1i):

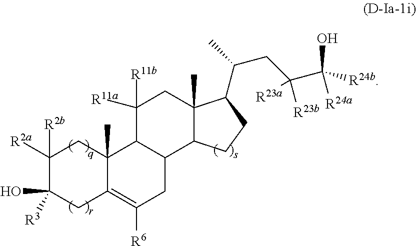

D-105. The compound according to embodiment D-103, wherein the compound of Formula (D-Ia-1) is a compound of Formula (D-Ia-1ii):

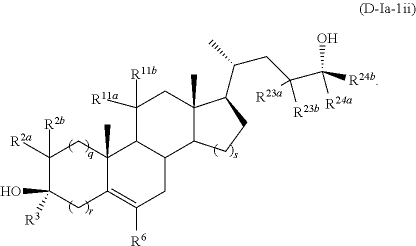

D-106. The compound according to embodiment D-103, wherein the compound of Formula (D-Ia) is a compound of Formula (D-Ia-2):

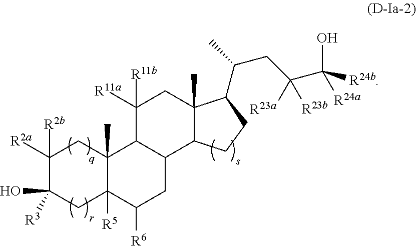

D-107. The compound according to embodiment D-106, wherein the compound of Formula (D-Ia-2) is a compound of Formula (D-Ia-2i):

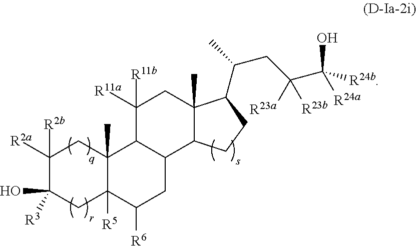

D-108. The compound according to embodiment D-106, wherein the compound of Formula (D-Ia-2) is a compound of Formula (D-Ia-2ii):

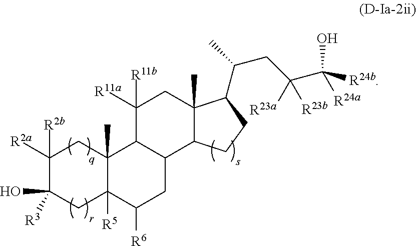

D-109. The compound according to embodiment D-102, wherein the compound of Formula (D-Ib) is a compound of Formula (D-Ib-1):

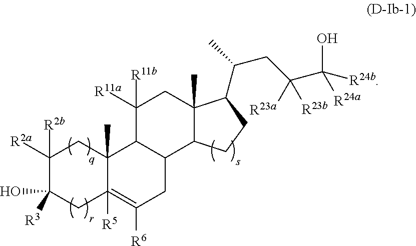

D-110. The compound according to embodiment D-109, wherein the compound of Formula (D-Ib-1) is a compound of Formula (D-Ib-1i):

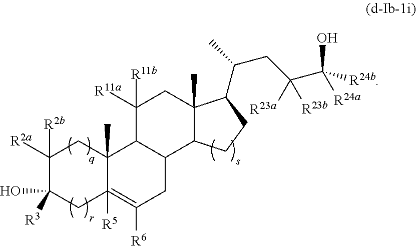

D-111. The compound according to embodiment D-109, wherein the compound of Formula (D-Ib-1) is a compound of Formula (D-Ib-1ii):

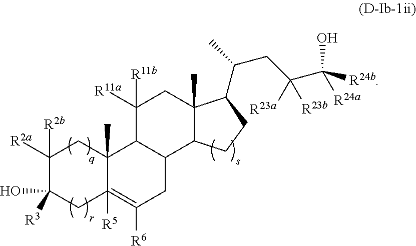

D-112. The compound according to embodiment D-102, wherein the compound of Formula (D-Ib) is a compound of Formula (D-Ib-2):

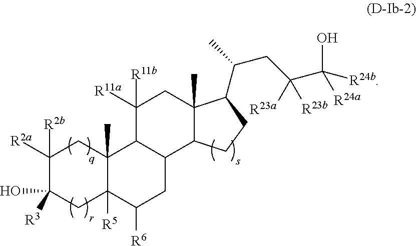

D-113. The compound according to embodiment D-112, wherein the compound of Formula (D-Ib-2) is a compound of Formula (D-Ib-2i):

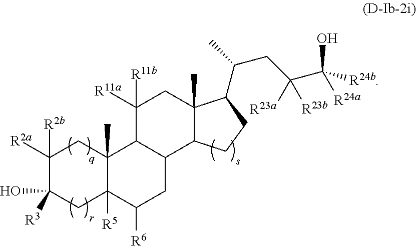

D-114. The compound according to embodiment D-112, wherein the compound of Formula (D-Ib-2) is a compound of Formula (D-Ib-2ii):

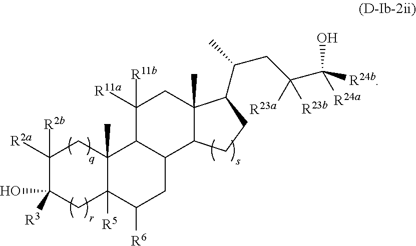

D-115. The compound according to embodiment D-101, wherein the compound of Formula (D-I) is a compound of Formula (D-II):

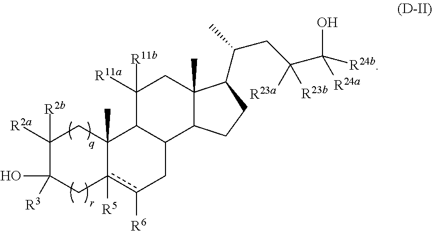

D-116. The compound according to embodiment D-115, wherein the compound of Formula (D-II) is a compound of Formula (D-IIa) or (D-LIb):

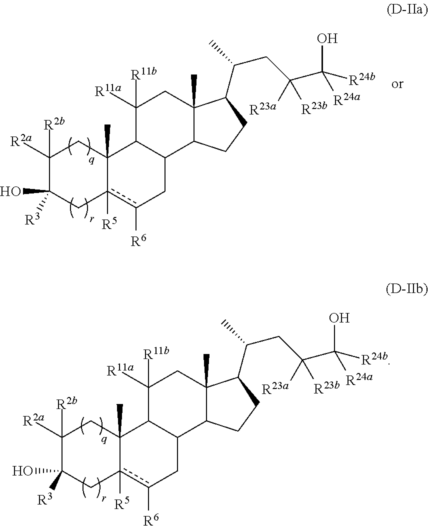

D-117. The compound according to embodiment D-116, wherein the compound of Formula (D-IIa) is a compound of Formula (D-IIa-1):

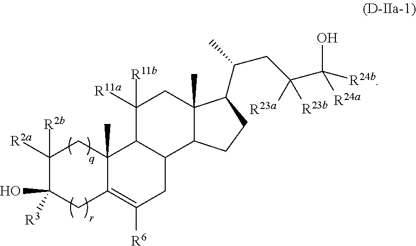

D-118. The compound according to embodiment D-117, wherein the compound of Formula (D-IIa-1) is a compound of Formula (D-Ia-1i):

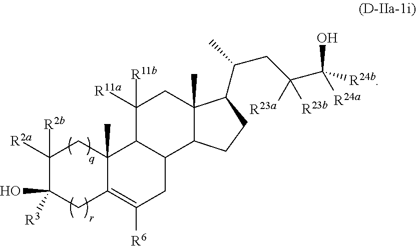

D-119. The compound according to embodiment D-117, wherein the compound of Formula (D-IIa-1) is a compound of Formula (D-IIa-1ii):

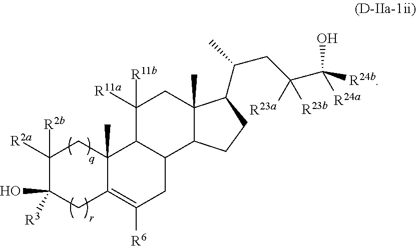

D-120. The compound according to embodiment D-116, wherein the compound of Formula (D-IIa) is a compound of Formula (D-IIa-2):

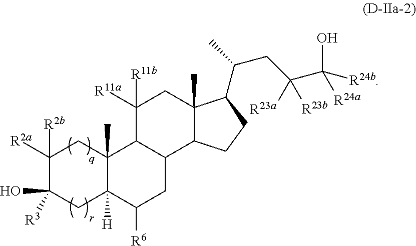

D-121. The compound according to embodiment D-120, wherein the compound of Formula (D-IIa-2) is a compound of Formula (D-Ia-2i):

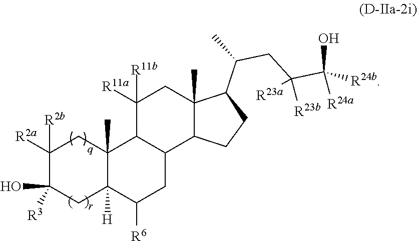

D-122. The compound according to embodiment D-120, wherein the compound of Formula (D-IIa-2) is a compound of Formula (D-IIa-2ii):

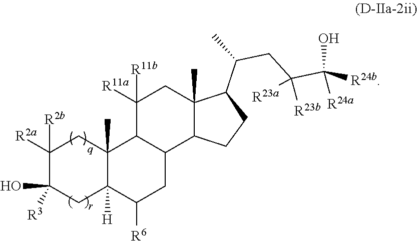

D-123. The compound according to embodiment D-116, wherein the compound of Formula (D-IIb) is a compound of Formula (D-LIb-1):

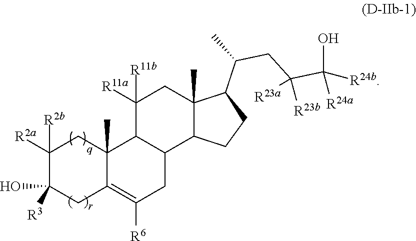

D-124. The compound according to embodiment D-123, wherein the compound of Formula (D-IIb-1) is a compound of Formula (D-IIb-1i):

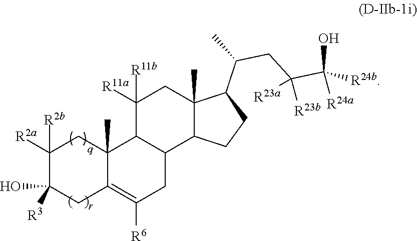

D-125. The compound according to embodiment D-123, wherein the compound of Formula (D-IIb-1) is a compound of Formula (D-IIb-1ii):

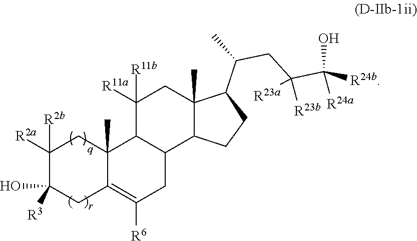

D-126. The compound according to embodiment D-116, wherein the compound of Formula (D-IIb) is a compound of Formula (D-LIb-2):

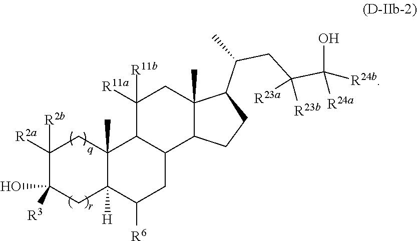

D-127. The compound according to embodiment D-126, wherein the compound of Formula (D-IIb-2) is a compound of Formula (D-IIb-2i):

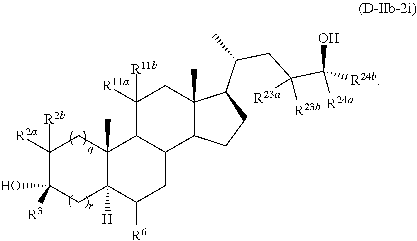

D-128. The compound according to embodiment D-126, wherein the compound of Formula (D-IIb-2) is a compound of Formula (D-IIb-2ii):

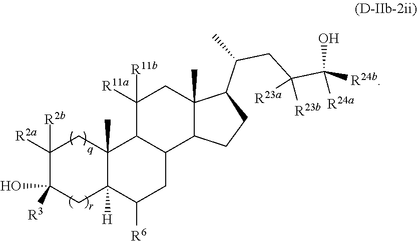

D-129. The compound according to embodiment D-101, wherein the compound of Formula (D-I) is a compound of Formula (D-III):

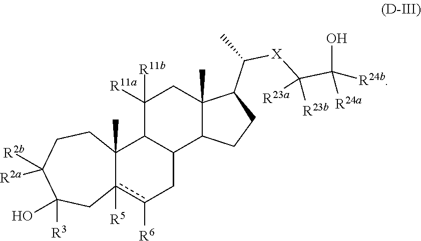

D-130. The compound according to embodiment D-129, wherein the compound of Formula (D-III) is a compound of Formula (D-IIIa) or Formula (D-IIIb):

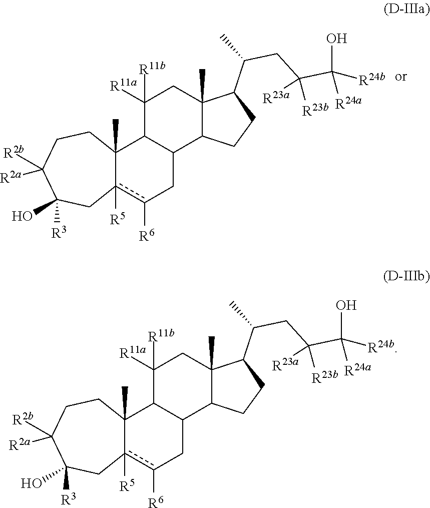

D-131. The compound according to embodiment D-130, wherein the compound of Formula (D-IIIa) is a compound of Formula (D-IIIa-1):

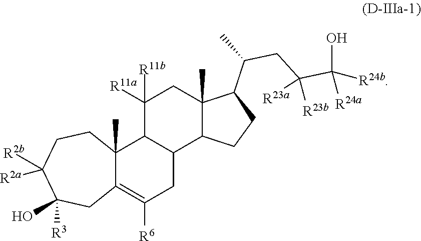

D-132. The compound according to embodiment D-131, wherein the compound of Formula (D-IIIa-1) is a compound of Formula (D-IIIa-1i):

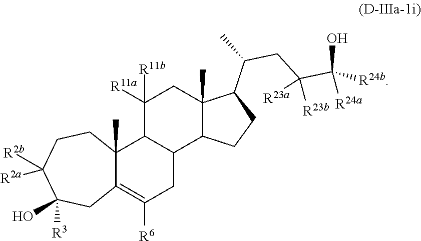

D-133. The compound according to embodiment D-131, wherein the compound of Formula (D-IIIa-1) is a compound of Formula (D-IIIa-1ii):

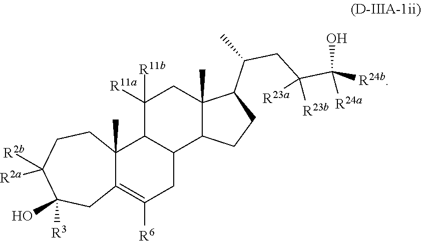

D-134. The compound according to embodiment D-130, wherein the compound of Formula (D-IIIa) is a compound of Formula (D-IIIa-2):

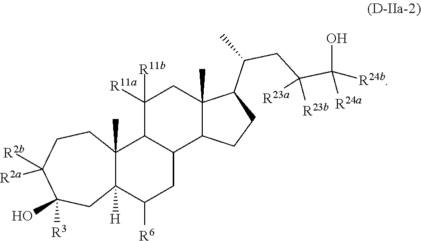

D-135. The compound according to embodiment D-134, wherein the compound of Formula (D-IIIa-2) is a compound of Formula (D-IIIa-2i):

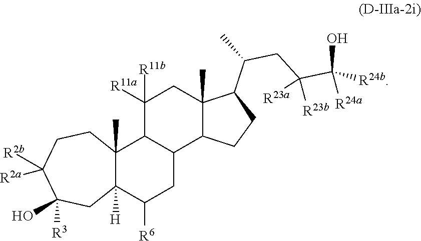

D-136. The compound according to embodiment D-134, wherein the compound of Formula (D-IIIa-2) is a compound of Formula (D-IIIa-2ii):

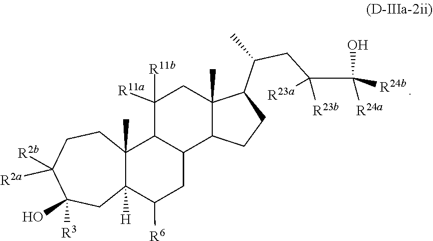

D-137. The compound according to embodiment D-130, wherein the compound of Formula (D-IIIb) is a compound of Formula (D-IIIb-1):

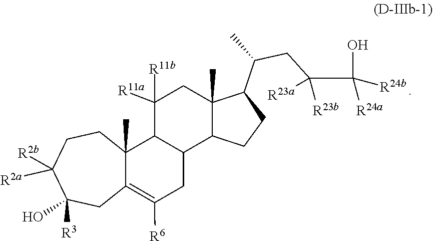

D-138. The compound according to embodiment D-137, wherein the compound of Formula (D-IIIb-1) is a compound of Formula (D-IIIb-1i):

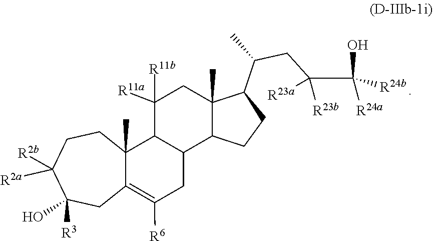

D-139. The compound according to embodiment D-137, wherein the compound of Formula (D-IIIb-1) is a compound of Formula (D-IIIb-1ii):

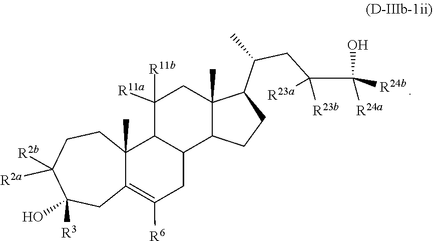

D-140. The compound according to embodiment D-130, wherein the compound of Formula (D-IIIb) is a compound of Formula (D-IIIb-2):

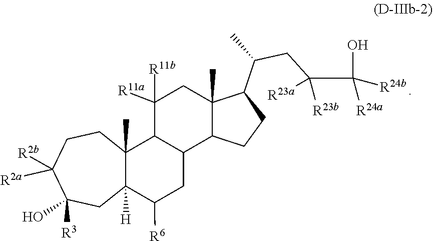

D-141. The compound according to embodiment D-140, wherein the compound of Formula (D-IIIb-2) is a compound of Formula (D-IIIb-2i):

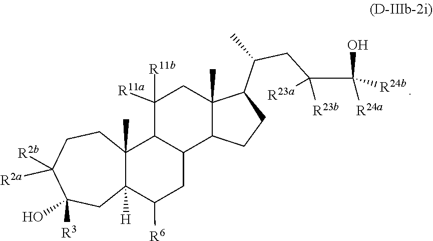

D-142. The compound according to embodiment D-140, wherein the compound of Formula (D-IIIb-2) is a compound of Formula (D-IIIb-2ii):

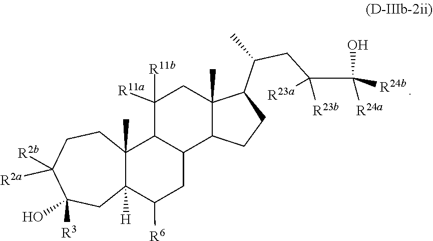

D-143. The compound according to embodiment D-101, wherein the compound of Formula (D-I) is a compound of Formula (D-IV):

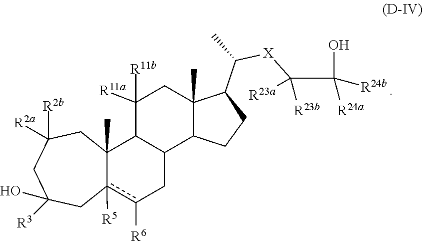

D-144. The compound according to embodiment D-143, wherein the compound of Formula (D-IV) is a compound of Formula (D-IVa) or Formula (D-IVb):

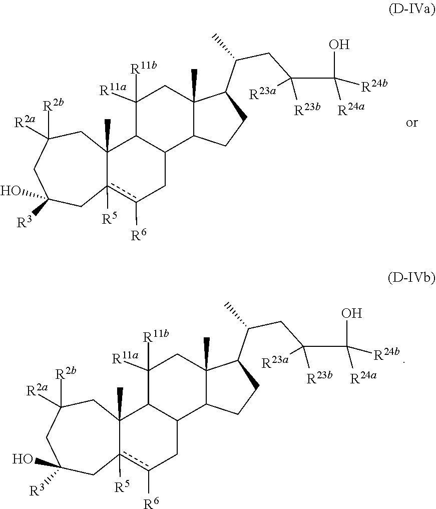

D-145. The compound according to embodiment D-144, wherein the compound of Formula (D-IVa) is a compound of Formula (D-IVa-1):

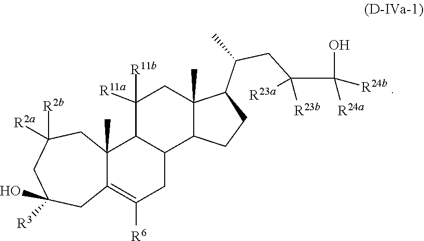

D-146. The compound according to embodiment D-145, wherein the compound of Formula (D-IVa-1) is a compound of Formula (D-IVa-1i):

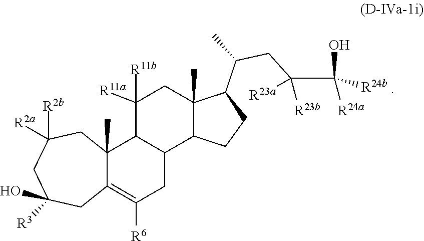

D-147. The compound according to embodiment D-145, wherein the compound of Formula (D-IVa-1) is a compound of Formula (D-IVa-1ii):

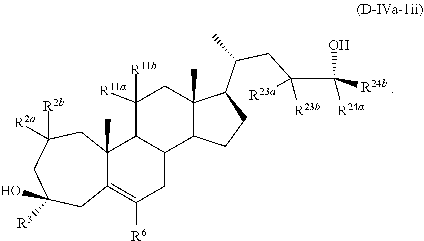

D-148. The compound according to embodiment D-144, wherein the compound of Formula (D-IVa) is a compound of Formula (D-IVa-2):

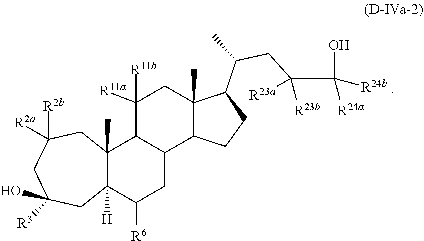

D-149. The compound according to embodiment D-148, wherein the compound of Formula (D-IVa-2) is a compound of Formula (D-IVa-2i):

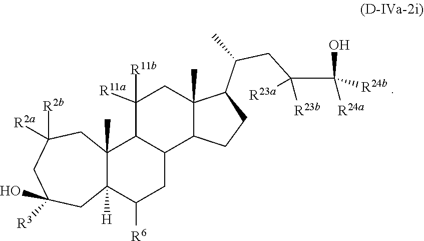

D-150. The compound according to embodiment D-148, wherein the compound of Formula (D-IVa-2) is a compound of Formula (D-IVa-2ii):

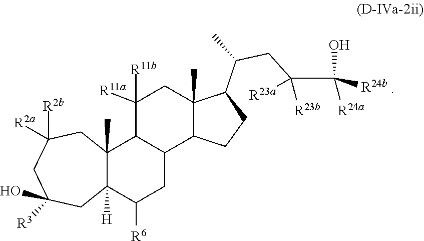

D-151. The compound according to embodiment D-144, wherein the compound of Formula (D-IVb) is a compound of Formula (D-IVb-1):

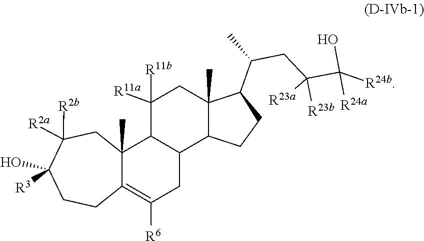

D-152. The compound according to embodiment D-151, wherein the compound of Formula (D-IVb-1) is a compound of Formula (D-IVb-1i):

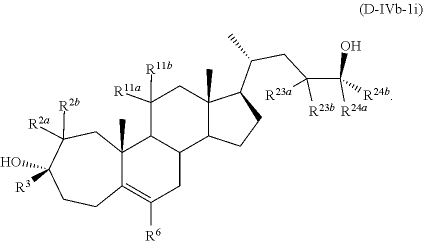

D-153. The compound according to embodiment D-151, wherein the compound of Formula (D-IVb-1) is a compound of Formula (D-IVb-1ii):

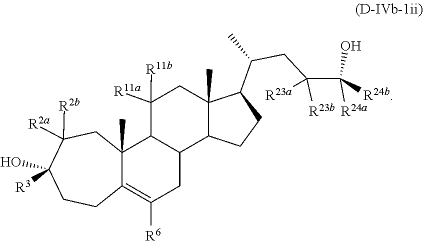

D-154. The compound according to embodiment D-144, wherein the compound of Formula (D-IVb) is a compound of Formula (D-IVb-2):

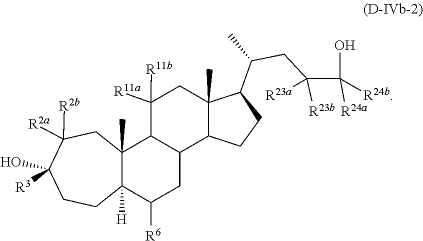

D-155. The compound according to embodiment D-154, wherein the compound of Formula (D-IVb-2) is a compound of Formula (D-IVb-2i):

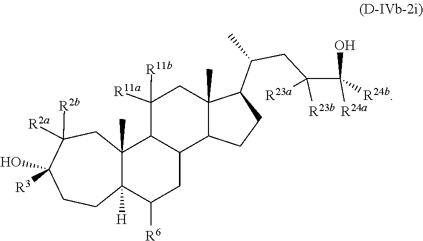

D-156. The compound according to embodiment D-154, wherein the compound of Formula (D-IVb-2) is a compound of Formula (D-IVb-2ii):

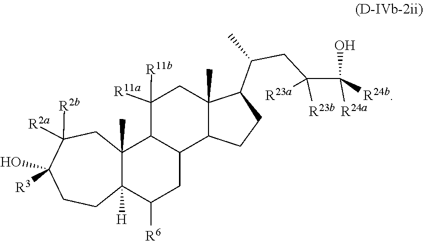

D-157. The compound according to any one of embodiments D-101 to D-156, wherein each of R^{2a} and R^{2b} is hydrogen.

D-158. The compound according to any one of embodiments D-101 to D-156, wherein R³ is unsubstituted C₁₋₆alkyl.

D-159. The compound according to embodiment D-158, wherein R³ is —CH₃, —CH₂CH₃, or —CH₂CH₂CH₃.

D-160. The compound according to embodiment D-159, wherein R³ is —CH₃.

D-161. The compound according to embodiment D-159, wherein R³ is —CH₂CH₃.

D-162. The compound according to any one of embodiments D-101 to D-156, wherein each of R^{11a} and R^{11b} is hydrogen.

D-163 The compound according to embodiments D-101 to D-102, D-106 to D-108, D-112 to D-116, D-129 to D-130, and D-143 to D-144, wherein R⁵, when present, is hydrogen.

D-164. The compound according to embodiments D-101 to D-102, D-106 to D-108, D-112 to D-116, D-129 to D-130, and D-143 to D-144, wherein R⁵, when present, is in the alpha configuration.

D-165. The compound according to embodiments D-101 to D-156, wherein R⁶ is hydrogen.

D-166. The compound according to any one of embodiments D-101 to D-156, wherein each of R^{23a} and R^{23b} is hydrogen.

D-167. The compound according to any one of embodiments D-101 to D-156, wherein at least one of R^{23a} and R^{23b} is fluorine.

D-168. The compound according to any one of embodiments D-101 to D-156, wherein each of R^{23a} and R^{23b} is fluorine.

D-169. The compound according to any one of embodiments D-101 to D-156, wherein R^{24a} is substituted C₁₋₆alkyl.

D-170. The compound according to embodiment D-169, wherein R^{24a} is C₁₋₆alkyl substituted with fluorine.

D-171. The compound according to embodiment D-170, wherein R^{24a} is —CF₃.

D-172. The compound according to any one of embodiments D-101 to D-156, wherein R^{24a} is unsubstituted C₁₋₆alkyl.

D-173. The compound according to embodiment D-172, wherein R^{24a} is unsubstituted-CH₃.

D-174. The compound according to any one of embodiments D-101 to D-156, wherein R^{24b} is unsubstituted C₁₋₆alkyl.

D-175. The compound according to any embodiment D-174, wherein R^{24b} is —CH₃.

D-176. The compound according to any one of embodiments D-101 to D-156, wherein R^{24b} is substituted C₁₋₆alkyl.

D-177. The compound according to embodiment D-176, wherein R^{24b} is —CF₃.

D-178. The compound according to any one of embodiments D-101 to D-156, wherein R^{24a} is —CF₃ and R^{24b} is —CH₃.

D-179. The compound according to any one of embodiments D-101 to D-156, wherein R^{24a} is —CH₃ and R^{24b} is —CH₃.

D-180. The compound according to any one of embodiments D-101 to D-156, wherein R^{24a} is —CH₃ and R^{24b} is —CF₃.

D-181. The compound or pharmaceutically acceptable salt thereof according to any one of embodiments D-101 to D-166, wherein q is 0 and r is 1.

D-182. The compound or pharmaceutically acceptable salt thereof according to any one of embodiments D-101 to D-138, wherein q is 1 and r is 2.

D-183. The compound or pharmaceutically acceptable salt thereof according to any one of embodiments D-101 to D-138, wherein q is 2 and r is 1.

D-184. The compound or pharmaceutically acceptable salt thereof according to any one of embodiments D-101 to D-138, wherein q is 2 and r is 2.

D-185. The compound or pharmaceutically acceptable salt thereof according to any one of embodiments D-101 to D-114, wherein s is 1.

D-186. The compound or pharmaceutically acceptable salt thereof according to any one of embodiments D-101 to D-114, wherein s is 2.

D-187. The compound or pharmaceutically acceptable salt thereof according to any one of embodiments D-101 to D-114, wherein s is 3.

D-188. The compound or pharmaceutically acceptable salt thereof according to any one of embodiments D-101 to D-114, wherein s is 4.

D-189. The compound according to embodiment D-101, wherein the compound of Formula (D-I) is selected from the group consisting of D-1, D-2, D-3, D-4, D-5, D-6, D-7, D-8, D-9, D-9a, D-10, D-11, D-12, D-13, D-14, D-15, D-16, and D-17.

D-190. The compound according to embodiment D-101, wherein the compound of Formula (D-I) is selected from the group consisting of D-1, D-2, D-3, D-4, D-5, D-6, D-7, D-8, D-9, D-10, D-11, D-12, D-13, D-14, D-15, D-16, and D-17.

D-191. A pharmaceutical composition comprising a compound according to any one of embodiments D-101 to D-190, and a pharmaceutically acceptable carrier.

D-192. A method for treating a CNS-related condition in a subject comprising administering to the subject an effective amount of a compound according to any one of embodiments D-101 to D-190, or a pharmaceutical composition according to embodiment D-191.

D-193. The method according to embodiment D-192, wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease) and tinnitus.

D-194. A method of inducing sedation or anesthesia in a subject, comprising administering to the subject an effective amount of a compound according to any one of embodiments D-101 to D-190, or a pharmaceutical composition according to embodiment D-191.

D-195. A compound according to any one of embodiments D-101 to D-190, or a pharmaceutical composition according to embodiment D-191, for use in treating a CNS-related condition in a subject.

D-196. The compound or pharmaceutical composition for use according to embodiment D-195, wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease) and tinnitus.

D-197. A compound according to any one of embodiments D-101 to D-190, or a pharmaceutical composition according to embodiment D-191, for use inducing sedation or anesthesia in a subject.

D-198. Use of a compound according to any one of embodiments D-101 to D-190, or a pharmaceutical composition according to embodiment D-191, for the manufacture of a medicament for treating a CNS-related condition in a subject.

D-199. The use according to embodiment D-198, wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease) and tinnitus.

D-200. Use of a compound according to any one of embodiments D-101 to D-190, or a pharmaceutical composition according to embodiment D-191, for the manufacture of a medicament for inducing sedation or anesthesia in a subject.

E-1. A compound of Formula (E-I):

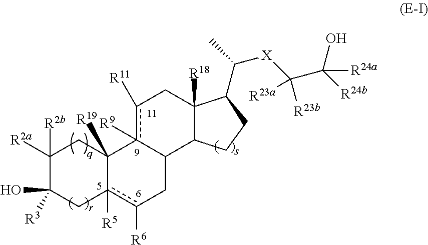

or a pharmaceutically acceptable salt thereof;
wherein:
   each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein R^{A2} is hydrogen or substituted or unsubstituted C₁₋₆alkyl;
   R³ is substituted or unsubstituted C₁₋₆alkyl, substituted or unsubstituted C₂₋₆alkenyl, or substituted or unsubstituted C₂₋₆alkynyl, or substituted or unsubstituted C₃₋₁₀carbocyclyl;
   [symbol] represents a single or double bond;
   when the bond between C9 and C11 is a double bond, R⁹ is absent and R¹¹ is selected from the group consisting of hydrogen, halogen, substituted or unsubstituted C₁₋₆alkyl, substituted or unsubstituted C₂₋₆alkenyl, substituted or unsubstituted C₂₋₆alkynyl, substituted or unsubstituted C₃₋₁₀carbocyclyl, substituted or unsubstituted C₁₋₆alkoxy, substituted or unsubstituted 3- to 10-membered heterocyclyl, —OH, and -amino;
   when the bond between C9 and C11 is a single bond, then R⁹ is hydrogen and R¹¹ is halogen or substituted or unsubstituted C₁₋₆alkyl; or R⁹ and R¹¹, taken together with the carbon atoms to which they are attached, form a substituted or unsubstituted C₃₋₆carbocyclyl ring;
   when the bond between C5 and C6 is a double bond, then R⁵ is absent and R⁶ is hydrogen, halogen, or substituted or unsubstituted C₁₋₆alkyl;
   when the bond between C5 and C6 is a single bond, then each of R⁵ and R⁶ is independently hydrogen, halogen, or substituted or unsubstituted C₁₋₆alkyl;
   R¹⁸ is hydrogen, substituted or unsubstituted C₁₋₆alkyl, or OR^{A18}, wherein R^{A18} is substituted or unsubstituted C₁₋₆alkyl;
   R¹⁹ is hydrogen, substituted or unsubstituted C₁₋₆alkyl, or OR^{A19}, wherein R^{A19} is substituted or unsubstituted C₁₋₆alkyl;
   X is —(C(R^{X})₂)ₙ— or —O—, wherein R^{X} is hydrogen or fluorine, or one R^{X} group and R^{23b} are joined to form a double bond;
   n is 1 or 2;
   each of R^{23a} and R^{23b} is independently hydrogen or fluorine;
   R^{24a} is selected from the group consisting of substituted or unsubstituted C₁₋₆-alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl;
   R^{24b} is hydrogen or a substituted or unsubstituted C₁₋₆alkyl; or
   R^{24a} and R^{24b}, taken together with the carbon atoms to which they are attached, form a substituted or unsubstituted C₃₋₆carbocyclyl, or substituted or unsubstituted 3- to 6-membered heterocyclyl;
   q is 0, 1 or 2;
   r is 1 or 2; and
   s is 1 or 2.

E-1A. A compound of Formula (E-Ia):

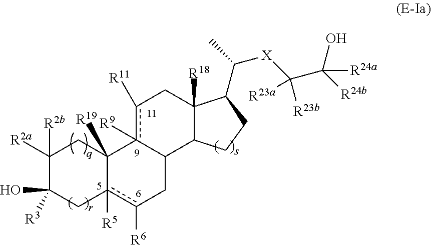

or a pharmaceutically acceptable salt thereof;
wherein:
   each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein R^{A2} is hydrogen or substituted or unsubstituted C₁₋₆alkyl;
   R³ is substituted or unsubstituted C₁₋₆alkyl, substituted or unsubstituted C₂₋₆alkenyl, substituted or unsubstituted C₂₋₆alkynyl, or substituted or unsubstituted C₃₋₁₀carbocyclyl;
   [symbol] represents a single or double bond;
   when the bond between C9 and C11 is a double bond, R⁹ is absent and R¹¹ is selected from the group consisting of hydrogen, halogen, substituted or unsubstituted C₁₋₆alkyl, substituted or unsubstituted C₂₋₆alkenyl, substituted or unsubstituted C₂₋₆alkynyl, substituted or unsubstituted C₃₋₁₀carbocyclyl, substituted or unsubstituted C₁₋₆alkoxy, substituted or unsubstituted 3- to 10-membered heterocyclyl, —OH, and -amino;
   when the bond between C9 and C11 is a single bond, then R⁹ is hydrogen and R¹¹ is halogen or substituted or unsubstituted C₁₋₆alkyl; or R⁹ and R¹¹, taken together with the carbon atoms to which they are attached, form a substituted or unsubstituted C₃₋₆carbocyclyl ring;
   R⁶ is hydrogen, substituted or unsubstituted C₁₋₆alkyl, or halogen; provided if a single bond is present between C5 and C6, then the hydrogen at C5 is in the alpha configuration;
   R¹⁸ is hydrogen, substituted or unsubstituted C₁₋₆alkyl, or OR^{A18}, wherein R^{A18} is substituted or unsubstituted C₁₋₆alkyl;
   R¹⁹ is hydrogen, substituted or unsubstituted C₁₋₆alkyl, or OR^{A19}, wherein R^{A19} is substituted or unsubstituted C₁₋₆alkyl;
X is —(C(R^{X})₂)ₙ— or —O—, wherein R^{X} is hydrogen or fluorine, or one R^{X} group and R^{23b} are joined to form a double bond;
   n is 1 or 2;
   each of R^{23a} and R^{23b} is independently hydrogen or fluorine;
   R^{24a} is selected from the group consisting of substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl;
   R^{24b} is hydrogen or substituted or unsubstituted C₁₋₆alkyl; or
   R^{24a} and R^{24b}, taken together with the carbon atom to which they are attached, form a substituted or unsubstituted C₃₋₆carbocyclyl or substituted or unsubstituted 3- to 6-membered heterocyclyl;
   q is 0, 1 or 2;
   r is 1 or 2; and
   s is 1 or 2.

E-2. The compound or pharmaceutically acceptable salt thereof according to embodiment E-1, wherein the compound of formula (E-I) is a compound of formula (E-I-A):

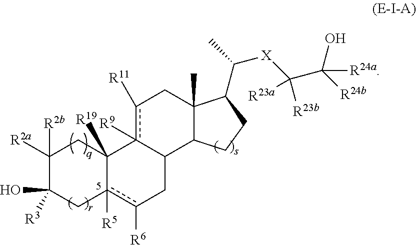

E-3. The compound or pharmaceutically acceptable salt thereof according to embodiment E-1, wherein the compound of formula (E-I) is a compound of formula (E-I-B):

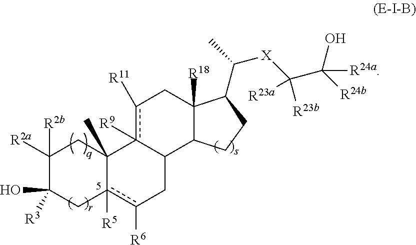

E-4. The compound or pharmaceutically acceptable salt thereof according to embodiment E-1, wherein the compound of formula (E-I) is a compound of formula (E-I-C):

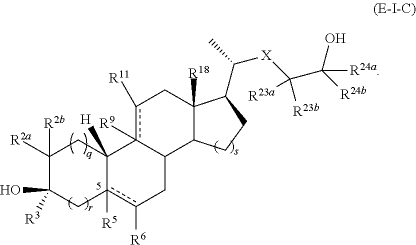

E-5. The compound or pharmaceutically acceptable salt thereof according to embodiment E-1, wherein the compound of formula (E-I) is a compound of formula (E-I-D):

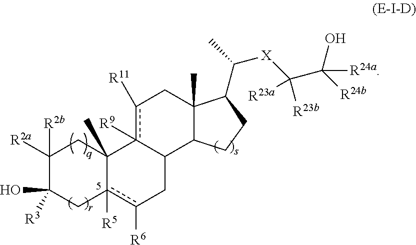

E-6. The compound or pharmaceutically acceptable salt thereof according to embodiment E-5, wherein the compound of formula (E-I-D) is a compound of formula (E-I-D-i):

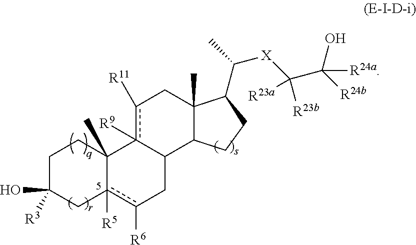

E-7. The compound or pharmaceutically acceptable salt thereof according to embodiment E-6, wherein the compound of formula (E-I-D-i) is a compound of formula (E-I-D-i-1):

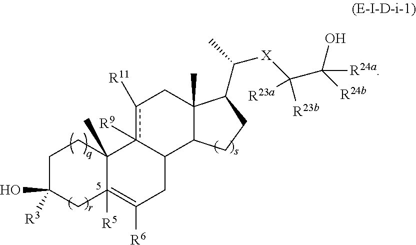

E-8. The compound or pharmaceutically acceptable salt thereof according to embodiment E-7, wherein the compound of formula (E-I-D-i-1) is a compound of formula (E-I-D-i-1a):

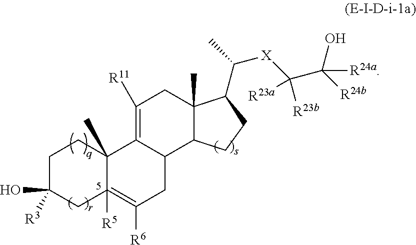

E-9. The compound or pharmaceutically acceptable salt thereof according to embodiment E-7, wherein the compound of formula (E-I-D-i-1) is a compound of formula (E-I-D-i-1b):

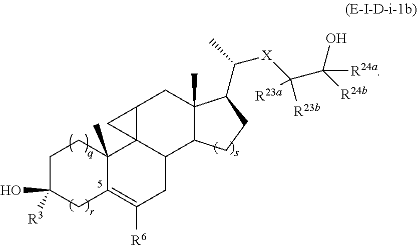

E-10. The compound or pharmaceutically acceptable salt thereof according to embodiment E-6, wherein the compound of formula (E-I-D-i) is a compound of formula (E-I-D-i-2):

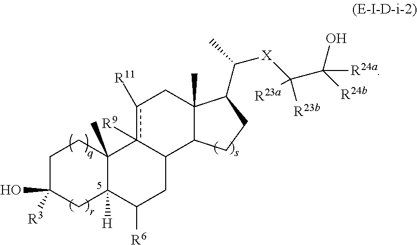

E-11. The compound or pharmaceutically acceptable salt thereof according to embodiment E-10, wherein the compound of formula (E-I-D-i-2) is a compound of formula (E-I-D-i-2a):

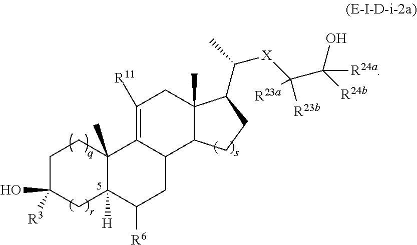

E-12. The compound or pharmaceutically acceptable salt thereof according to embodiment E-10, wherein the compound of formula (E-I-D-i-2) is a compound of formula (E-I-D-i-2b):

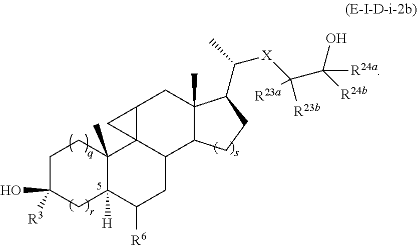

E-13. The compound or pharmaceutically acceptable salt thereof according to embodiment E-1, wherein the compound of formula (E-I) is a compound of formula (E-I-E):

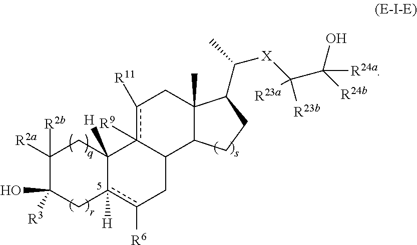

E-14. The compound or pharmaceutically acceptable salt thereof according to embodiment E-13, wherein the compound of formula (E-I-E) is a compound of formula (E-I-E-i):

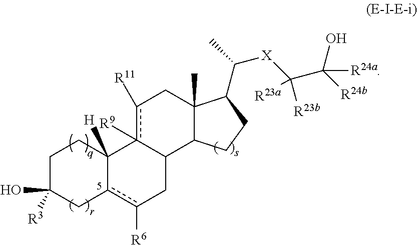

E-15. The compound or pharmaceutically acceptable salt thereof according to embodiment E-14, wherein the compound of formula (E-I-E-i) is a compound of formula (E-I-E-i-1):

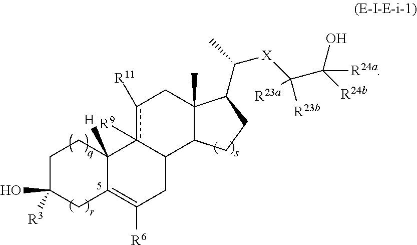

E-16. The compound or pharmaceutically acceptable salt thereof according to embodiment E-15, wherein the compound of formula (E-I-E-i-1) is a compound of formula (E-I-E-i-1a):

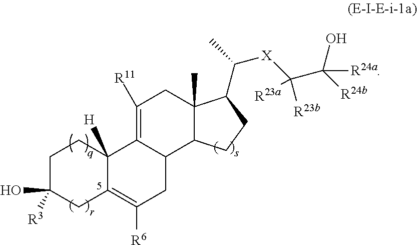

E-17. The compound or pharmaceutically acceptable salt thereof according to embodiment E-15, wherein the compound of formula (E-I-E-i-1) is a compound of formula (E-I-E-i-1b):

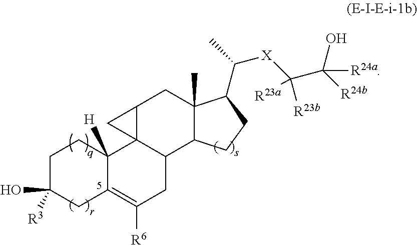

E-18. The compound or pharmaceutically acceptable salt thereof according to embodiment E-14, wherein the compound of formula (E-I-E-i) is a compound of formula (E-I-E-i-2):

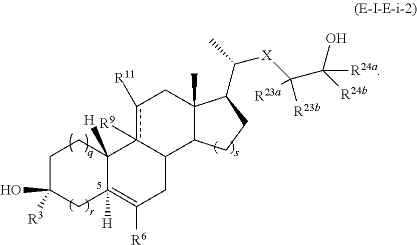

E-19. The compound or pharmaceutically acceptable salt thereof according to embodiment E-18, wherein the compound of formula (E-I-E-i-2) is a compound of formula (E-I-E-i-2a):

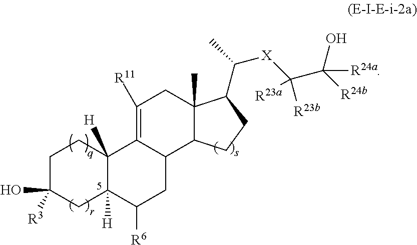

E-20. The compound or pharmaceutically acceptable salt thereof according to embodiment E-18, wherein the compound of formula (E-I-E-i-2) is a compound of formula (E-I-E-i-2b):

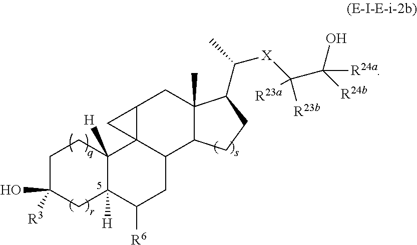

E-21. The compound or pharmaceutically acceptable salt thereof according to embodiment E-1, wherein the compound of formula (E-I) is a compound of formula (E-II):

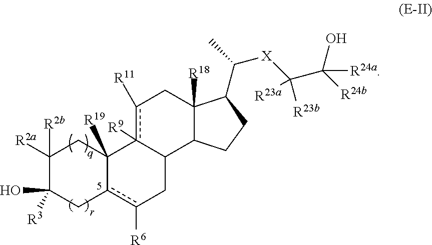

E-22. The compound or pharmaceutically acceptable salt thereof according to embodiment E-21, wherein the compound of formula (E-II) is a compound of formula (E-II-A):

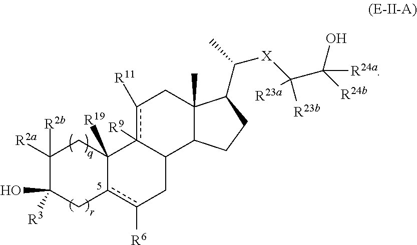

E-23. The compound or pharmaceutically acceptable salt thereof according to embodiment E-21, wherein the compound of formula (E-II) is a compound of formula (E-II-B):

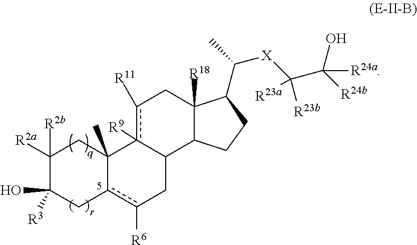

E-24. The compound or pharmaceutically acceptable salt thereof according to embodiment E-21, wherein the compound of formula (E-II) is a compound of formula (E-II-C):

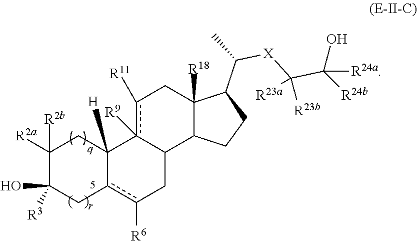

E-25. The compound or pharmaceutically acceptable salt thereof according to embodiment E-21, wherein the compound of formula (E-II) is a compound of formula (E-II-D):

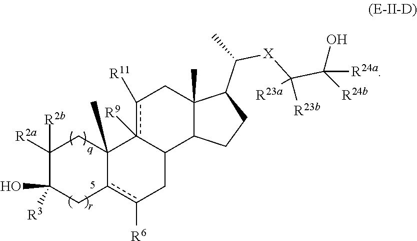

E-26. The compound or pharmaceutically acceptable salt thereof according to embodiment E-25, wherein the compound of formula (E-II-D) is a compound of formula (E-II-D-i):

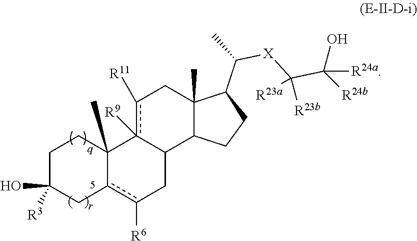

E-27. The compound or pharmaceutically acceptable salt thereof according to embodiment E-26, wherein the compound of formula (E-II-D-i) is a compound of formula (E-II-D-i-1):

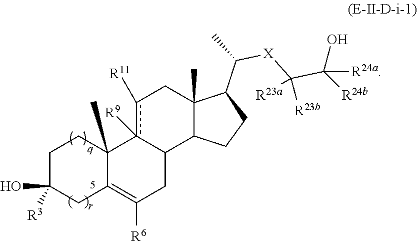

E-28. The compound or pharmaceutically acceptable salt thereof according to embodiment E-27, wherein the compound of formula (E-II-D-i-1) is a compound of formula (E-II-D-i-1a):

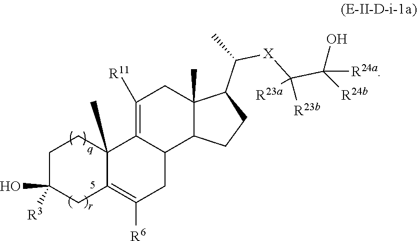

E-29. The compound or pharmaceutically acceptable salt thereof according to embodiment E-27, wherein the compound of formula (E-II-D-i-1) is a compound of formula (E-II-D-i-1b):

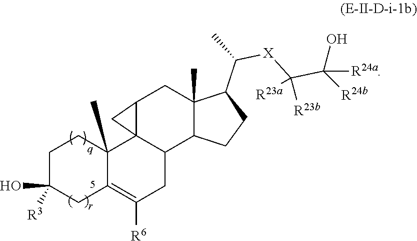

E-30. The compound or pharmaceutically acceptable salt thereof according to embodiment E-26, wherein the compound of formula (E-II-D-i) is a compound of formula (E-II-D-i-2):

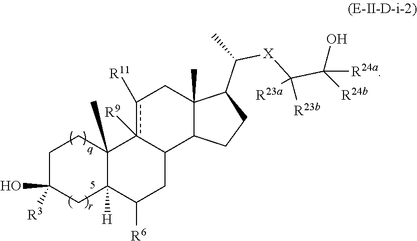

E-31. The compound or pharmaceutically acceptable salt thereof according to embodiment E-30, wherein the compound of formula (E-II-D-i-2) is a compound of formula (E-II-D-i-2a):

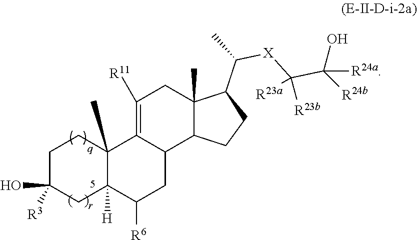

E-32. The compound or pharmaceutically acceptable salt thereof according to embodiment E-30, wherein the compound of formula (E-II-D-i-2) is a compound of formula (E-II-D-i-2b):

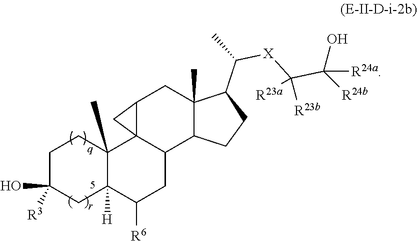

E-33. The compound or pharmaceutically acceptable salt thereof according to embodiment E-21, wherein the compound of formula (E-II) is a compound of formula (E-II-E):

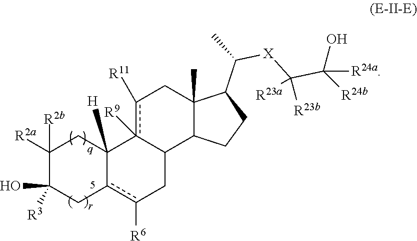

E-34. The compound or pharmaceutically acceptable salt thereof according to embodiment E-33, wherein the compound of formula (E-II-E) is a compound of formula (E-II-E-i):

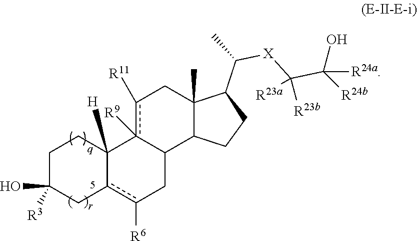

E-35. The compound or pharmaceutically acceptable salt thereof according to embodiment E-34, wherein the compound of formula (E-II-E-i) is a compound of formula (E-II-E-i-1):

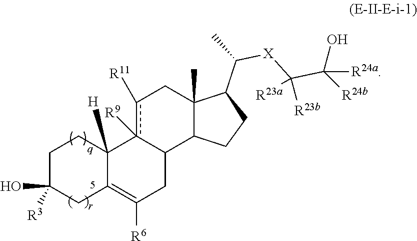

E-36. The compound or pharmaceutically acceptable salt thereof according to embodiment E-35, wherein the compound of formula (E-II-E-i-1) is a compound of formula (E-II-E-i-1a):

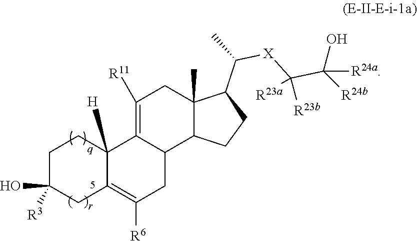

E-37. The compound or pharmaceutically acceptable salt thereof according to embodiment E-35, wherein the compound of formula (E-II-E-i-1) is a compound of formula (E-II-E-i-1b):

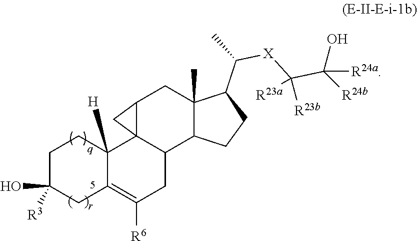

E-38. The compound or pharmaceutically acceptable salt thereof according to embodiment E-34, wherein the compound of formula (E-II-E-i) is a compound of formula (E-II-E-i-2):

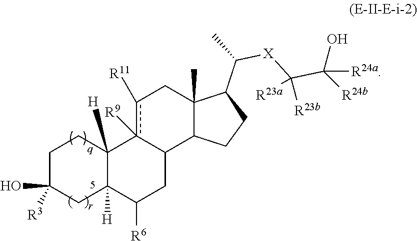

E-39. The compound or pharmaceutically acceptable salt thereof according to embodiment E-38, wherein the compound of formula (E-II-E-i-2) is a compound of formula (E-II-E-i-2a):

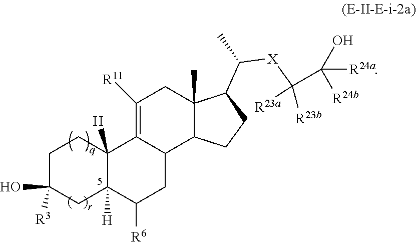

E-40. The compound or pharmaceutically acceptable salt thereof according to embodiment E-38, wherein the compound of formula (E-II-E-i-2) is a compound of formula (E-II-E-i-2b):

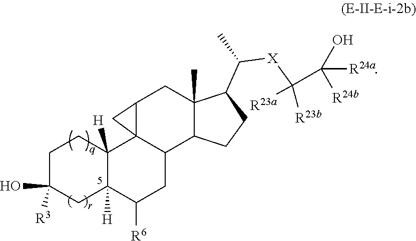

E-41. The compound or pharmaceutically acceptable salt thereof according to embodiment E-1, wherein the compound of formula (E-I) is a compound of formula (E-III):

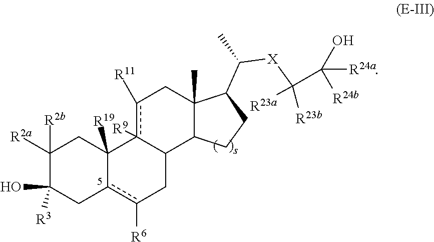

E-42. The compound or pharmaceutically acceptable salt thereof according to embodiment E-41, wherein the compound of formula (E-III) is a compound of formula (E-III-A):

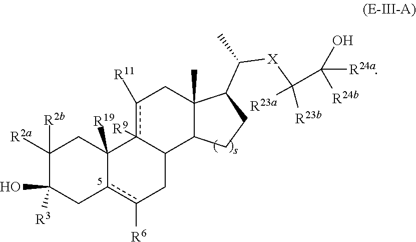

E-43. The compound or pharmaceutically acceptable salt thereof according to embodiment E-41, wherein the compound of formula (E-III) is a compound of formula (E-III-B):

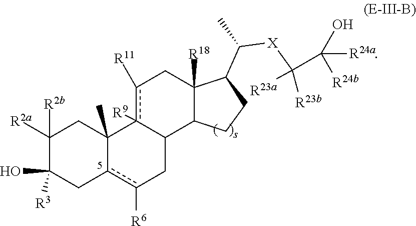

E-44. The compound or pharmaceutically acceptable salt thereof according to embodiment E-41, wherein the compound of formula (E-III) is a compound of formula (E-III-C):

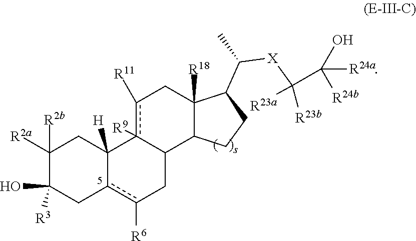

E-45. The compound or pharmaceutically acceptable salt thereof according to embodiment E-41, wherein the compound of formula (E-III) is a compound of formula (E-III-D):

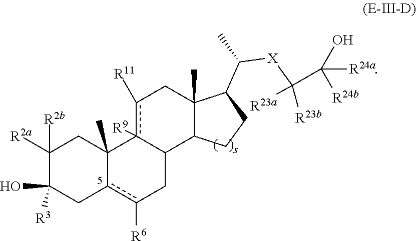

E-46. The compound or pharmaceutically acceptable salt thereof according to embodiment E-45, wherein the compound of formula (E-III-D) is a compound of formula (E-III-D-i):

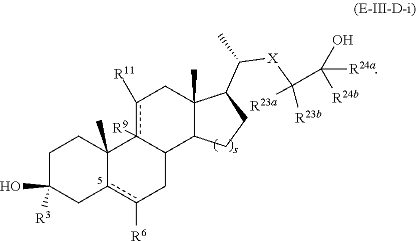

E-47. The compound or pharmaceutically acceptable salt thereof according to embodiment E-46, wherein the compound of formula (E-III-D-i) is a compound of formula (E-III-D-i-1):

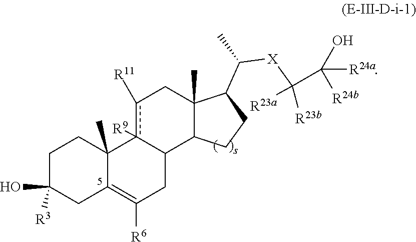

E-48. The compound or pharmaceutically acceptable salt thereof according to embodiment E-47, wherein the compound of formula (E-III-D-i-1) is a compound of formula (E-III-D-i-1a):

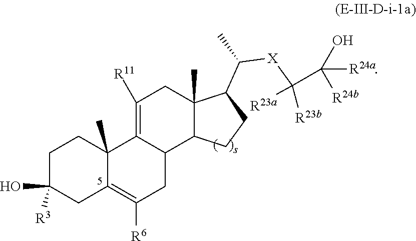

E-49. The compound or pharmaceutically acceptable salt thereof according to embodiment E-47, wherein the compound of formula (E-III-D-i-1) is a compound of formula (E-III-D-i-1b):

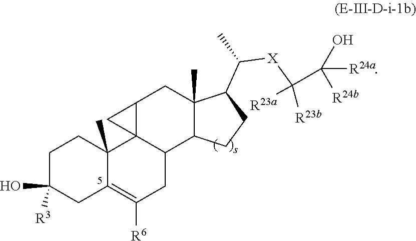

E-50. The compound or pharmaceutically acceptable salt thereof according to embodiment E-46, wherein the compound of formula (E-III-D-i) is a compound of formula (E-III-D-i-2):

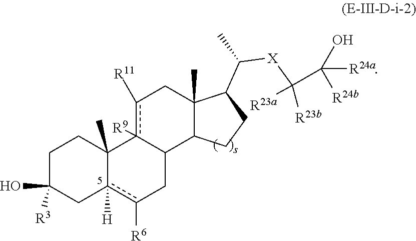

E-51. The compound or pharmaceutically acceptable salt thereof according to embodiment E-50, wherein the compound of formula (E-III-D-i-2) is a compound of formula (E-III-D-i-2a):

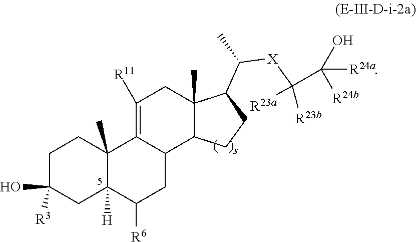

E-52. The compound or pharmaceutically acceptable salt thereof according to embodiment E-50, wherein the compound of formula (E-III-D-i-2) is a compound of formula (E-III-D-i-2b):

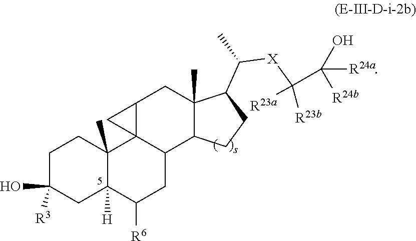

E-53. The compound or pharmaceutically acceptable salt thereof according to embodiment E-41, wherein the compound of formula (E-III) is a compound of formula (E-III-E):

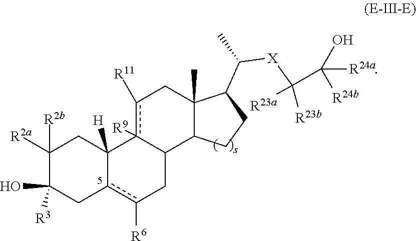

E-54. The compound or pharmaceutically acceptable salt thereof according to embodiment E-53, wherein the compound of formula (E-III-E) is a compound of formula (E-III-E-i):

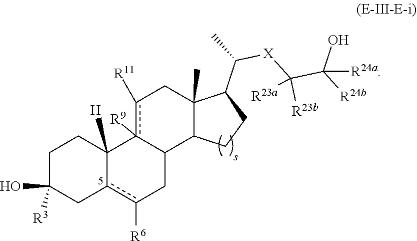

E-55. The compound or pharmaceutically acceptable salt thereof according to embodiment E-54, wherein the compound of formula (E-III-E-i) is a compound of formula (E-III-E-i-1):

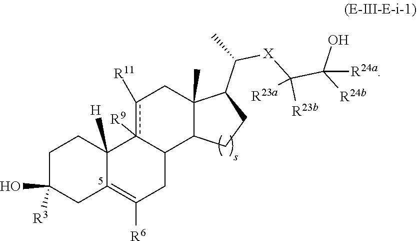

E-56. The compound or pharmaceutically acceptable salt thereof according to embodiment E-55, wherein the compound of formula (E-III-E-i-1) is a compound of formula (E-III-E-i-1a):

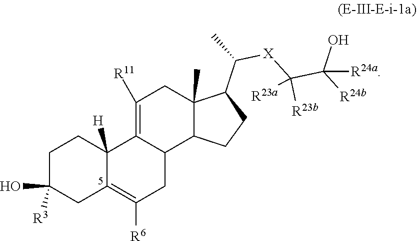

E-57. The compound or pharmaceutically acceptable salt thereof according to embodiment E-55, wherein the compound of formula (E-III-E-i-1) is a compound of formula (E-III-E-i-1b):

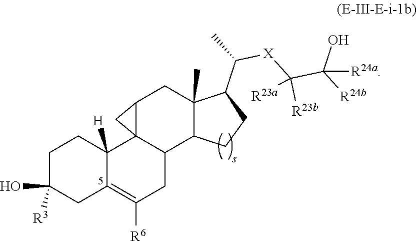

E-58. The compound or pharmaceutically acceptable salt thereof according to embodiment E-54, wherein the compound of formula (E-III-E-i) is a compound of formula (E-III-E-i-2):

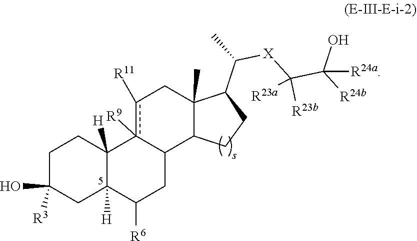

E-59. The compound or pharmaceutically acceptable salt thereof according to embodiment E-58, wherein the compound of formula (E-III-E-i-2) is a compound of formula (E-III-E-i-2a):

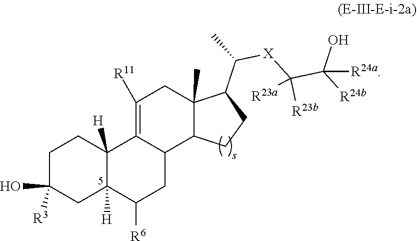

E-60. The compound or pharmaceutically acceptable salt thereof according to embodiment E-58, wherein the compound of formula (E-III-E-i-2) is a compound of formula (E-III-E-i-2b):

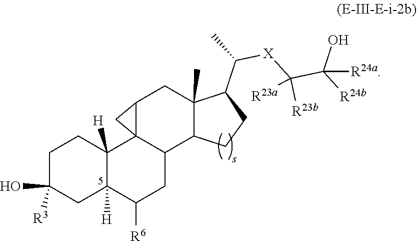

E-61. The compound or pharmaceutically acceptable salt thereof according to embodiment E-1, wherein the compound of formula (E-I) is a compound of formula (E-IV):

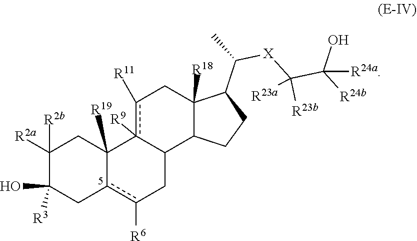

E-62. The compound or pharmaceutically acceptable salt thereof according to embodiment E-61, wherein the compound of formula (E-IV) is a compound of formula (E-IV-A):

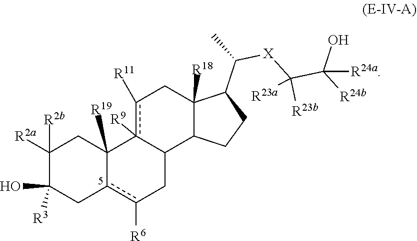

E-63. The compound or pharmaceutically acceptable salt thereof according to embodiment E-61, wherein the compound of formula (E-IV) is a compound of formula (E-IV-B):

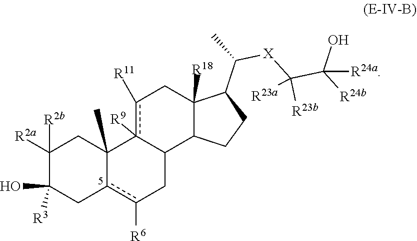

E-64. The compound or pharmaceutically acceptable salt thereof according to embodiment E-61, wherein the compound of formula (E-IV) is a compound of formula (E-IV-C):

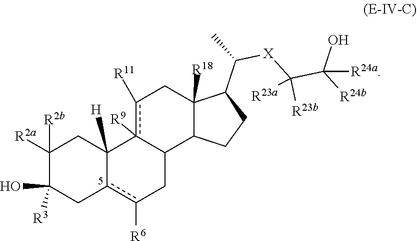

E-65. The compound or pharmaceutically acceptable salt thereof according to embodiment E-61, wherein the compound of formula (E-IV) is a compound of formula (E-IV-D):

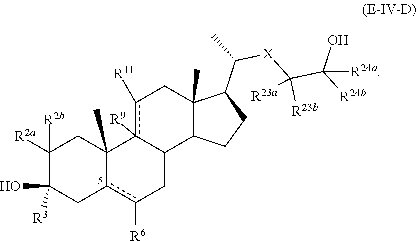

E-66. The compound or pharmaceutically acceptable salt thereof according to embodiment E-65, wherein the compound of formula (E-IV-D) is a compound of formula (E-IV-D-i):

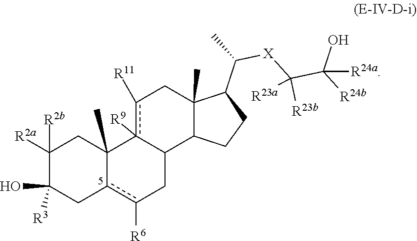

E-67. The compound or pharmaceutically acceptable salt thereof according to embodiment E-66, wherein the compound of formula (E-IV-D-i) is a compound of formula (E-IV-D-i-1):

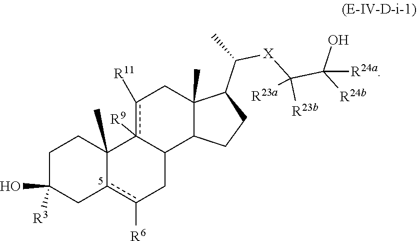

E-68. The compound or pharmaceutically acceptable salt thereof according to embodiment E-67, wherein the compound of formula (E-IV-D-i-1) is a compound of formula (E-IV-D-i-1a):

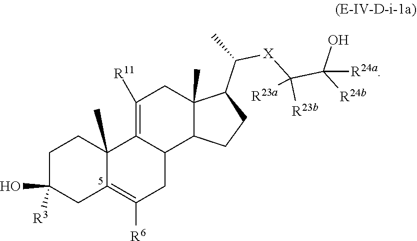

E-69. The compound or pharmaceutically acceptable salt thereof according to embodiment E-67, wherein the compound of formula (E-IV-D-i-1) is a compound of formula (E-IV-D-i-1b):

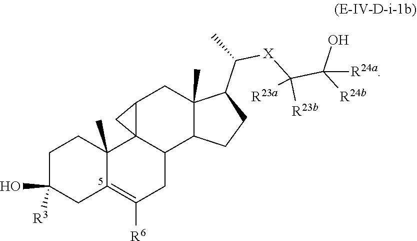

E-70. The compound or pharmaceutically acceptable salt thereof according to embodiment E-66, wherein the compound of formula (E-IV-D-i) is a compound of formula (E-IV-D-i-2):

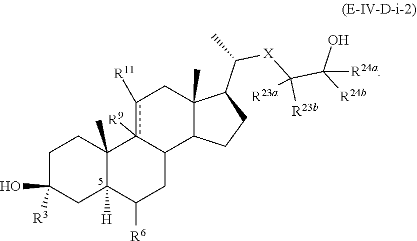

E-71. The compound or pharmaceutically acceptable salt thereof according to embodiment E-70, wherein the compound of formula (E-IV-D-i-2) is a compound of formula (E-IV-D-i-2a):

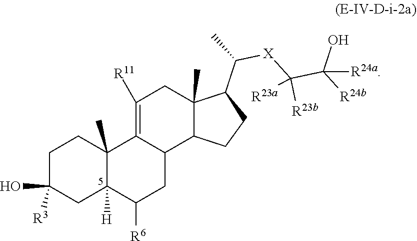

E-72. The compound or pharmaceutically acceptable salt thereof according to embodiment E-70, wherein the compound of formula (E-IV-D-i-2) is a compound of formula (E-IV-D-i-2b):

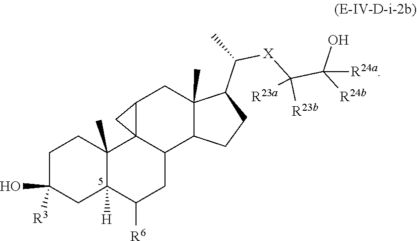

E-73. The compound or pharmaceutically acceptable salt thereof according to embodiment E-61, wherein the compound of formula (E-IV) is a compound of formula (E-IV-E):

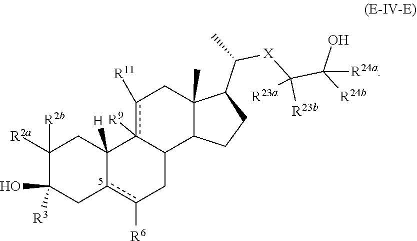

E-74. The compound or pharmaceutically acceptable salt thereof according to embodiment E-73, wherein the compound of formula (E-IV-E) is a compound of formula (E-IV-E-i):

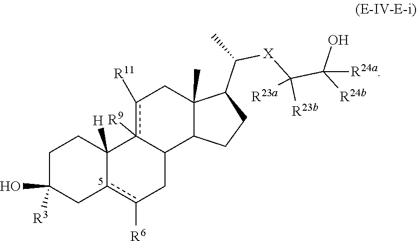

E-75. The compound or pharmaceutically acceptable salt thereof according to embodiment E-74, wherein the compound of formula (E-IV-E-i) is a compound of formula (E-IV-E-i-1):

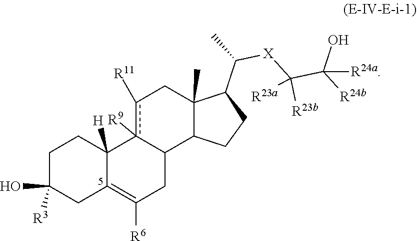

E-76. The compound or pharmaceutically acceptable salt thereof according to embodiment E-75, wherein the compound of formula (E-IV-E-i-1) is a compound of formula (E-IV-E-i-1a):

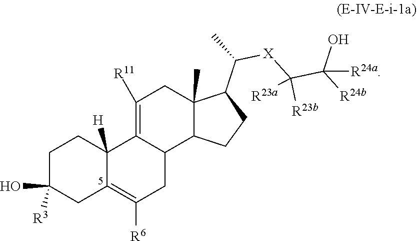

E-77. The compound or pharmaceutically acceptable salt thereof according to embodiment E-75, wherein the compound of formula (E-IV-E-i-1) is a compound of formula (E-IV-E-i-1b):

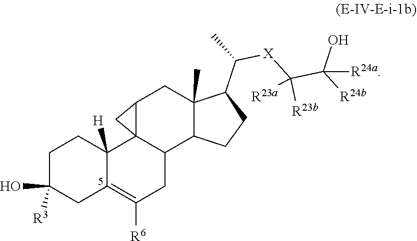

E-78. The compound or pharmaceutically acceptable salt thereof according to embodiment E-74, wherein the compound of formula (E-IV-E-i) is a compound of formula (E-IV-E-i-2):

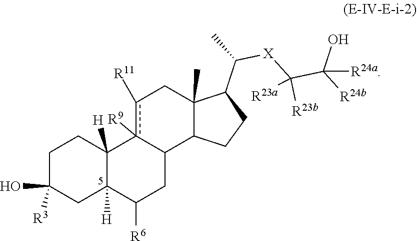

E-79. The compound or pharmaceutically acceptable salt thereof according to embodiment E-78, wherein the compound of formula (E-IV-E-i-2) is a compound of formula (E-IV-E-i-2a):

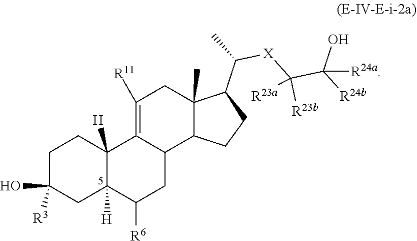

E-80. The compound or pharmaceutically acceptable salt thereof according to embodiment E-78, wherein the compound of formula (E-IV-E-i-2) is a compound of formula (E-IV-E-i-2b):

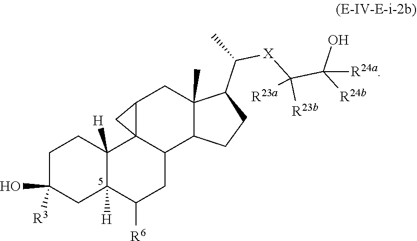

E-81. The compound or pharmaceutically acceptable salt thereof according to any one of embodiments E-1 to E-80, wherein R³ is unsubstituted C₁₋₆alkyl.

E-82. The compound or pharmaceutically acceptable salt thereof according to embodiment E-81, wherein R³ is —CH₃, —CH₂CH₃, or —CH₂CH₂CH₃.

E-83. The compound or pharmaceutically acceptable salt thereof according to embodiment E-82, wherein R³ is —CH₂CH₃.

E-84. The compound or pharmaceutically acceptable salt thereof according to any one of embodiments E-1 to E-80, wherein R⁶ is hydrogen.

E-85. The compound or pharmaceutically acceptable salt thereof according to any one of embodiments E-1 to E-7, E-10, E-13 to E-15, E-18, E-20, E-21 to E-27, E-30, E-33 to E-35, E-38, E-41 to E-47, E-50, E-53 to E-55, E-58, E-61 to E-67, E-70, E-73 to E-75 and E-78, wherein the bond between C9 and C11 is a double bond, R⁹ is absent and R¹¹ is selected from the group consisting of hydrogen, halogen, substituted or unsubstituted C₁₋₆alkyl, and substituted or unsubstituted C₁₋₆alkoxy.

E-86. The compound or pharmaceutically acceptable salt thereof according to embodiment E-85, wherein R¹¹ is hydrogen.

E-87. The compound or pharmaceutically acceptable salt thereof according to any one of embodiments E-1 to E-7, E-10, E-13 to E-15, E-18, E-20, E-21 to E-27, E-30, E-33 to E-35, E-38, E-41 to E-47, E-50, E-53 to E-55, E-58, E-61 to E-67, E-70, E-73 to E-75 and E-78, wherein the bond between C9 and C11 is a single bond, R⁹ is hydrogen and R¹¹ is selected from the group consisting of halogen or substituted or unsubstituted C₁₋₆alkyl.

E-88. The compound or pharmaceutically acceptable salt thereof according to any one of embodiments E-1 to E-8, E-10 to E-11, E-13 to E-16, E-18 to E-19, E-21 to E-28, E-30 to E-31, E-33 to E-36, E-38 to E-39, E-41 to E-48, E-50 to E-51, E-53 to E-56, E-58, E-61 to E-68, E-70 to E-71, E-73 to E-76, and E-78 to E-79, R¹¹ is selected from the group consisting of hydrogen, halogen, substituted or unsubstituted C₁₋₆alkyl, and substituted or unsubstituted C₁₋₆alkoxy.

E-89. The compound or pharmaceutically acceptable salt thereof according to embodiment E-88, wherein R¹¹ is hydrogen.

E-90. The compound or pharmaceutically acceptable salt thereof according to any one of embodiments E-1 to E-80, wherein X is —(C(R^{X})₂)ₙ—, n is 1 or 2 and R^{X} is hydrogen.

E-91. The compound or pharmaceutically acceptable salt thereof according to embodiment E-90, wherein n is 1.

E-92. The compound or pharmaceutically acceptable salt thereof according to embodiment E-90, wherein n is 2.

E-93. The compound or pharmaceutically acceptable salt thereof according to any one of embodiments E-1 to E-80, wherein each of R^{23a} and R^{23b} is independently hydrogen.

E-94. The compound or pharmaceutically acceptable salt thereof according to any one of embodiments E-1 to E-80, wherein each of R^{23a} and R^{23b} is independently fluorine.

E-95. The compound or pharmaceutically acceptable salt thereof according to any one of embodiments E-1 to E-80, wherein R^{24a} is substituted or unsubstituted C₁₋₆ alkyl.

E-96. The compound or pharmaceutically acceptable salt thereof according to embodiment E-95, wherein R^{24a} is unsubstituted C₁₋₆alkyl.

E-97. The compound or pharmaceutically acceptable salt thereof according to embodiment E-95, wherein R^{24a} is C₁₋₆alkyl substituted with fluorine.

E-98. The compound or pharmaceutically acceptable salt thereof according to embodiment E-95, wherein R^{24a} is —CH₃, —CH(CH₃)₂, or —CH₂CF₃.

E-99. The compound or pharmaceutically acceptable salt thereof according to any one of embodiments E-1 to E-80, wherein R^{24b} is hydrogen or substituted or unsubstituted C₁₋₆alkyl.

E-100. The compound or pharmaceutically acceptable salt thereof according to embodiment E-99, wherein R^{24b} is hydrogen.

E-101. The compound or pharmaceutically acceptable salt thereof according to embodiment E-99, wherein R^{24b} is unsubstituted C₁₋₆alkyl.

E-102. The compound or pharmaceutically acceptable salt thereof according to embodiment E-99, wherein R^{24b} is C₁₋₆alkyl substituted with fluorine.

E-103. The compound or pharmaceutically acceptable salt thereof according to embodiment E-99, wherein R^{24b} is —CH₃, —CH(CH₃)₂, or —CH₂CF₃.

E-104. The compound or pharmaceutically acceptable salt thereof according to any one of embodiments E-1 to E-80, wherein R^{24a} and R^{24b}, taken together with the carbon atom to which they are attached, form an unsubstituted C₃₋₆carbocyclyl.

E-105. The compound or pharmaceutically acceptable salt thereof according to embodiment E-104, wherein R^{24a} and R^{24b}, taken together with the carbon atom to which they are attached, form a cyclopropyl or cyclopentyl.

E-106. The compound or pharmaceutically acceptable salt thereof according to any one of embodiments E-1 to E-80, wherein R^{24a} and R^{24b}, taken together with the carbon atom to which they are attached, form an unsubstituted 3- to 6-membered heterocyclyl.

E-107. The compound or pharmaceutically acceptable salt thereof according to embodiment E-106, wherein R^{24a} and R^{24b}, taken together with the carbon atom to which they are attached, form a tetrahydrofuranyl.

E-108. The compound or pharmaceutically acceptable salt thereof according to embodiment E-1, wherein the compound of Formula (E-I) is selected from the group consisting of E-1-E-16 of Table E-1, or a pharmaceutically acceptable salt thereof.

E-109. The compound or pharmaceutically acceptable salt thereof according to embodiment E-1, wherein the compound of Formula (E-I) is selected from the group consisting of Compounds C-26-C-35 of Table E-1, or a pharmaceutically acceptable salt thereof.

E-110. The compound or pharmaceutically acceptable salt thereof according to embodiment E-1, wherein the compound of Formula (E-I) is selected from the group consisting of Compounds E-3, E-4, E-6, E-9, E-11, E-12 or pharmaceutically acceptable salts thereof.

E-111. A pharmaceutical composition comprising a compound or pharmaceutically acceptable salt thereof according to any one of embodiments E-1 to E-110, and a pharmaceutically acceptable carrier.

E-112. A method for treating a CNS-related condition in a subject, comprising administering to the subject an effective amount of a compound or pharmaceutically acceptable salt thereof according to any one of embodiments E-1 to E-110, or a pharmaceutical composition according to embodiment E-111.

E-113. The method according to embodiment E-112, wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease) and tinnitus.

E-114. A method of inducing sedation or anesthesia in a subject, comprising administering to the subject an effective amount of a compound or pharmaceutically acceptable salt thereof according to any one of embodiments E-1 to E-110, or a pharmaceutical composition according to embodiment E-111.

E-115. A compound or pharmaceutically acceptable salt thereof according to any one of embodiments E-1 to E-110, or a pharmaceutical composition according to embodiment E-111, for use in treating a CNS-related condition in a subject.

E-116. The compound or pharmaceutically acceptable salt thereof, or pharmaceutical composition for use according to embodiment E-115, wherein the wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease) and tinnitus.

E-117. A compound or pharmaceutically acceptable salt thereof according to any one of embodiments E-1 to E-110, or a pharmaceutical composition according to embodiment E-111, for use inducing sedation or anesthesia in a subject.

E-118. Use of a compound or pharmaceutically acceptable salt thereof according to any one of embodiments E-1 to E-110, or a pharmaceutical composition according to embodiment E-111, for the manufacture of a medicament for treating a CNS-related condition in a subject.

E-119. The use according to embodiment E-118, wherein the wherein the wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease) and tinnitus.

E-120. Use of a compound or pharmaceutically acceptable salt thereof according to any one of embodiments E-1 to E-110, or a pharmaceutical composition according to embodiment E-111, for the manufacture of a medicament for inducing sedation or anesthesia in a subject.

E-121. A compound of Formula (E-I):

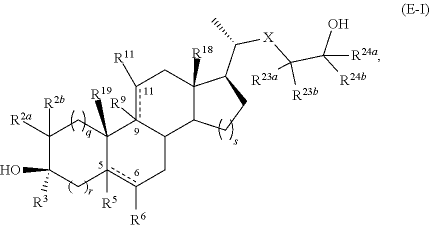

wherein:
   each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein RAZ is hydrogen or substituted or unsubstituted C₁₋₆alkyl;
   R³ is substituted or unsubstituted C₁₋₆alkyl, substituted or unsubstituted C₂₋₆alkenyl, or substituted or unsubstituted C₂₋₆alkynyl, or substituted or unsubstituted C₃₋₁₀carbocyclyl;
   [symbol] represents a single or double bond;
   when the bond between C9 and C11 is a double bond, R⁹ is absent and R¹¹ is selected from the group consisting of hydrogen, halogen, substituted or unsubstituted C₁₋₆alkyl, substituted or unsubstituted C₂₋₆alkenyl, substituted or unsubstituted C₂₋₆alkynyl, substituted or unsubstituted C₃₋₁₀carbocyclyl, substituted or unsubstituted C₁₋₆alkoxy, substituted or unsubstituted 3- to 10-membered heterocyclyl, —OH, and -amino;
   when the bond between C9 and C11 is a single bond, then R⁹ is hydrogen and R¹¹ is halogen or substituted or unsubstituted C₁₋₆alkyl; or R⁹ and R¹¹, taken together with the carbon atoms to which they are attached, form a substituted or unsubstituted C₃₋₆carbocyclyl ring;
   when the bond between C5 and C6 is a double bond, then R⁵ is absent and R⁶ is hydrogen, halogen, or substituted or unsubstituted C₁₋₆alkyl;
   when the bond between C5 and C6 is a single bond, then each of R⁵ and R⁶ is independently hydrogen, halogen, or substituted or unsubstituted C₁₋₆alkyl;
   R¹⁸ is hydrogen, substituted or unsubstituted C₁₋₆alkyl, or OR^{A18}, wherein R^{A18} is substituted or unsubstituted C₁₋₆alkyl;
   R¹⁹ is hydrogen, substituted or unsubstituted C₁₋₆alkyl, or OR^{A19}, wherein R^{A19} is substituted or unsubstituted C₁₋₆alkyl;
   X is —(C(R^{X})₂)ₙ— or —O—, wherein R^{X} is hydrogen or fluorine, or one R^{X} group and R^{23b} are joined to form a double bond;
   n is 1 or 2;
   each of R^{23a} and R^{23b} is independently hydrogen or fluorine;
   R^{24a} is selected from the group consisting of substituted or unsubstituted C₁₋₆ alkyl, substituted or unsubstituted C₂₋₆alkenyl, substituted or unsubstituted C₂₋₆ alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl;
   R^{24b} is hydrogen or a substituted or unsubstituted C₁₋₆alkyl; or
   R^{24a} and R^{24b}, taken together with the carbon atoms to which they are attached, form a substituted or unsubstituted C₃₋₆carbocyclyl, or substituted or unsubstituted 3 to 6-membered heterocyclyl;
   q is 0, 1 or 2;
   r is 1 or 2; and
   s is 1 or 2.

E-121A. A compound of Formula (E-Ia):

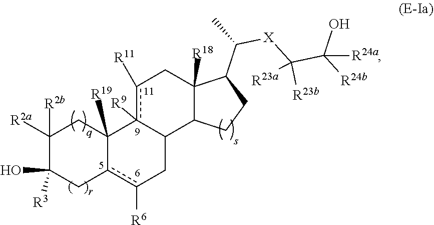

wherein:
   each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein R^{A2} is hydrogen or substituted or unsubstituted C₁₋₆alkyl;
   R³ is substituted or unsubstituted C₁₋₆alkyl, substituted or unsubstituted C₂₋₆alkenyl, substituted or unsubstituted C₂₋₆alkynyl, or substituted or unsubstituted C₃₋₁₀carbocyclyl;
   [symbol] represents a single or double bond;
   when the bond between C9 and C11 is a double bond, R⁹ is absent and R¹¹ is selected from the group consisting of hydrogen, halogen, substituted or unsubstituted C₁₋₆alkyl, substituted or unsubstituted C₂₋₆alkenyl, substituted or unsubstituted C₂₋₆alkynyl, substituted or unsubstituted C₃₋₁₀carbocyclyl, substituted or unsubstituted C₁₋₆alkoxy, substituted or unsubstituted 3- to 10-membered heterocyclyl, —OH, and -amino;
   when the bond between C9 and C11 is a single bond, then R⁹ is hydrogen and R¹¹ is halogen or substituted or unsubstituted C₁₋₆alkyl; or R⁹ and R¹¹, taken together with the carbon atoms to which they are attached, form a substituted or unsubstituted C₃₋₆carbocyclyl ring;
   R⁶ is hydrogen, substituted or unsubstituted C₁₋₆alkyl, or halogen; provided if a single bond is present between C5 and C6, then the hydrogen at C5 is in the alpha configuration;
   R¹⁸ is hydrogen, substituted or unsubstituted C₁₋₆alkyl, or OR^{A18}, wherein R^{A18} is substituted or unsubstituted C₁₋₆alkyl;
   R¹⁹ is hydrogen, substituted or unsubstituted C₁₋₆alkyl, or OR^{A19}, wherein R^{A19} is substituted or unsubstituted C₁₋₆alkyl;
   X is —(C(R^{X})₂)ₙ— or —O—, wherein R^{X} is hydrogen or fluorine, or one R^{X} group and R^{23b} are joined to form a double bond;
   n is 1 or 2;
   each of R^{23a} and R^{23b} is independently hydrogen or fluorine;
   R^{24a} is selected from the group consisting of substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl;
   R^{24b} is hydrogen or substituted or unsubstituted C₁₋₆alkyl; or
   R^{24a} and R^{24b}, taken together with the carbon atom to which they are attached, form a substituted or unsubstituted C₃₋₆carbocyclyl or substituted or unsubstituted 3- to 6-membered heterocyclyl;
   q is 0, 1 or 2;
   r is 1 or 2; and
   s is 1 or 2.

E-122. The compound according to embodiment E-121, wherein the compound of formula (E-I) is a compound of formula (E-I-A):

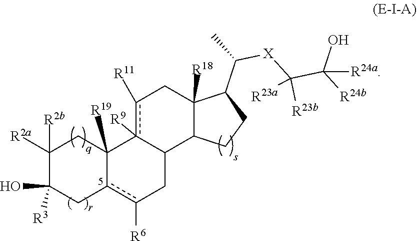

E-123. The compound according to embodiment E-121, wherein the compound of formula (E-I) is a compound of formula (E-I-B):

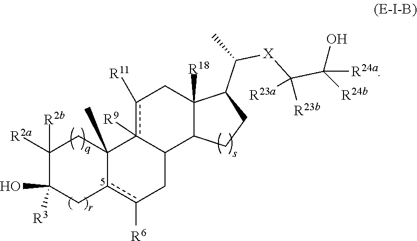

E-124. The compound according to embodiment E-121, wherein the compound of formula (E-I) is a compound of formula (E-I-C):

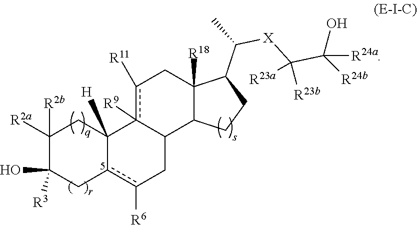

E-125. The compound according to embodiment E-121, wherein the compound of formula (E-I) is a compound of formula (E-I-D):

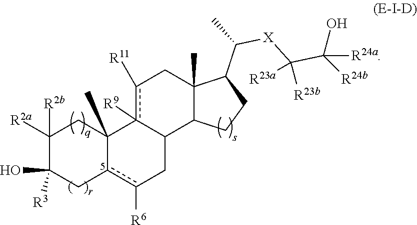

E-126. The compound according to embodiment E-125, wherein the compound of formula (E-I-D) is a compound of formula (E-I-D-i):

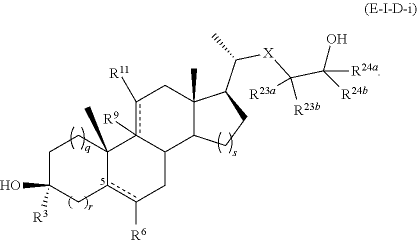

E-127. The compound according to embodiment E-126, wherein the compound of formula (E-I-D-i) is a compound of formula (E-I-D-i-1):

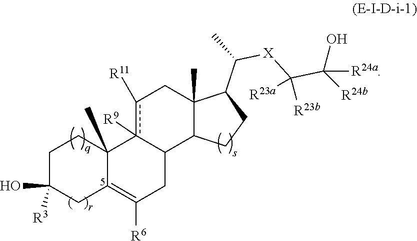

E-128. The compound according to embodiment E-127, wherein the compound of formula (E-I-D-i-1) is a compound of formula (E-I-D-i-1a):

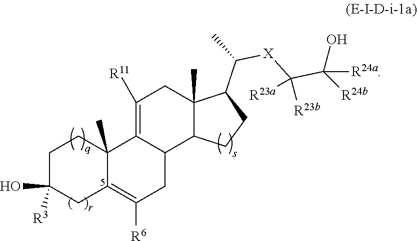

E-129. The compound according to embodiment E-127, wherein the compound of formula (E-I-D-i-1) is a compound of formula (E-I-D-i-1b):

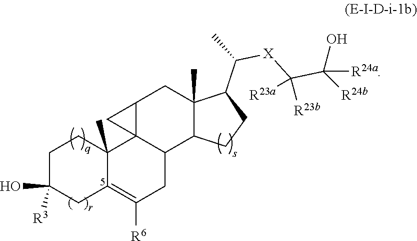

E-130. The compound according to embodiment E-126, wherein the compound of formula (E-I-D-i) is a compound of formula (E-I-D-i-2):

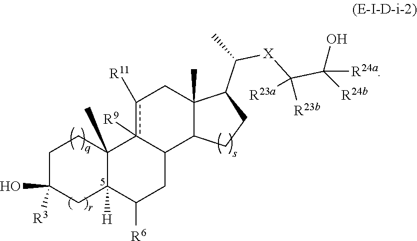

E-131. The compound according to embodiment E-130, wherein the compound of formula (E-I-D-i-2) is a compound of formula (E-I-D-i-2a):

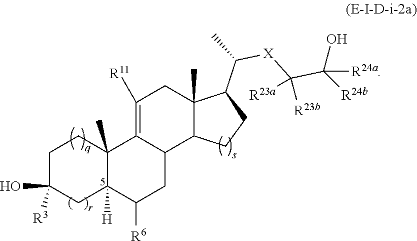

E-132. The compound according to embodiment E-130, wherein the compound of formula (E-I-D-i-2) is a compound of formula (E-I-D-i-2b):

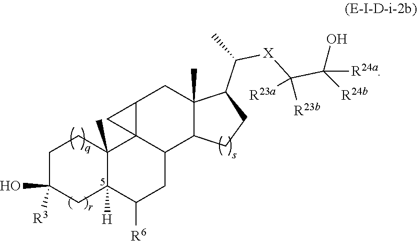

E-133. The compound according to embodiment E-121, wherein the compound of formula (E-I) is a compound of formula (E-I-E):

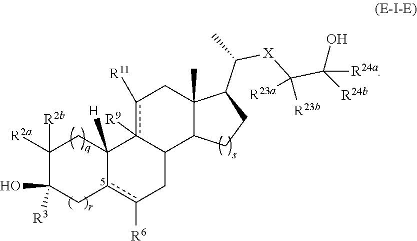

E-134. The compound according to embodiment E-133, wherein the compound of formula (E-I-E) is a compound of formula (E-I-E-i):

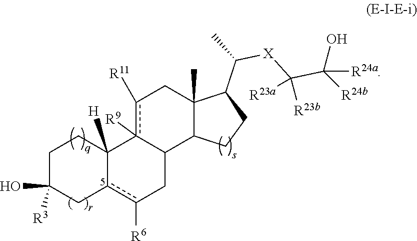

E-135. The compound according to embodiment E-134, wherein the compound of formula (E-I-E-i) is a compound of formula (E-I-E-i-1):

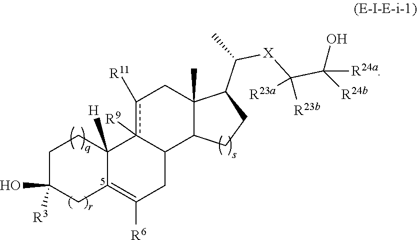

E-136. The compound according to embodiment E-135, wherein the compound of formula (E-I-E-i-1) is a compound of formula (E-I-E-i-1a):

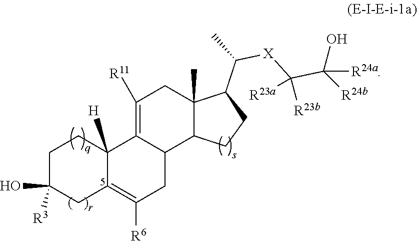

E-137. The compound according to embodiment E-135, wherein the compound of formula (E-I-E-i-1) is a compound of formula (E-I-E-i-1b):

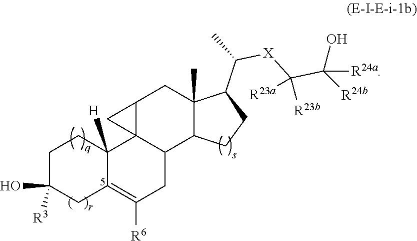

E-138. The compound according to embodiment E-134, wherein the compound of formula (E-I-E-i) is a compound of formula (E-I-E-i-2):

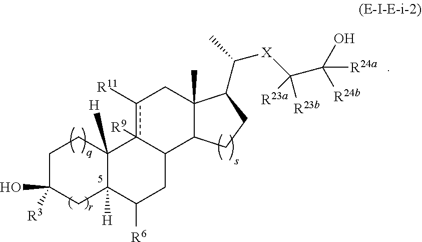

E-139. The compound according to embodiment E-138, wherein the compound of formula (E-I-E-i-2) is a compound of formula (E-I-E-i-2a):

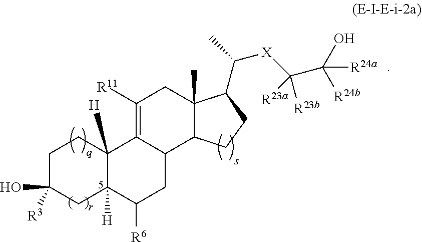

E-140. The compound according to embodiment E-138, wherein the compound of formula (E-I-E-i-2) is a compound of formula (E-I-E-i-2b):

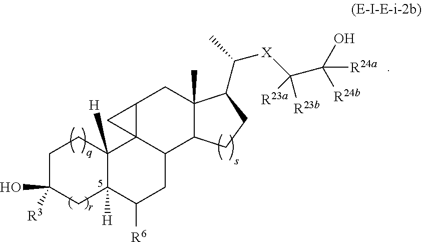

E-141. The compound according to embodiment E-121, wherein the compound of formula (E-I) is a compound of formula (E-II):

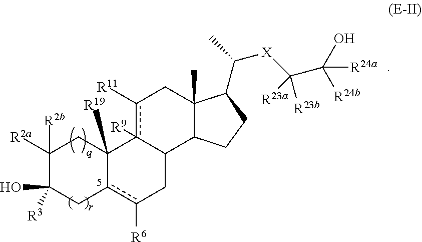

E-142. The compound according to embodiment E-141, wherein the compound of formula (E-II) is a compound of formula (E-II-A):

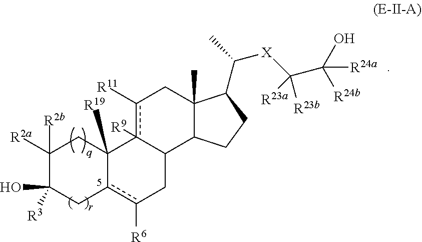

E-143. The compound according to embodiment E-141, wherein the compound of formula (E-II) is a compound of formula (E-II-B):

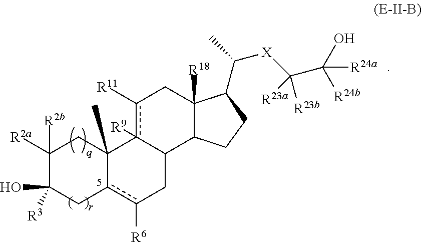

E-144. The compound according to embodiment E-141, wherein the compound of formula (E-II) is a compound of formula (E-II-C):

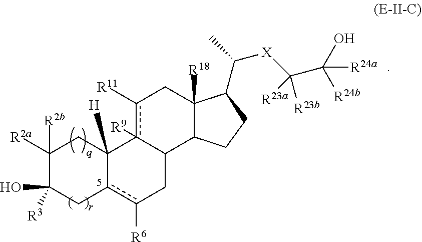

E-145. The compound according to embodiment E-141, wherein the compound of formula (E-II) is a compound of formula (E-II-D):

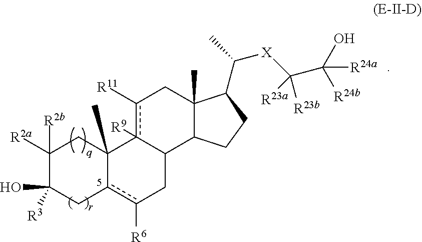

E-146. The compound according to embodiment E-145, wherein the compound of formula (E-II-D) is a compound of formula (E-II-D-i):

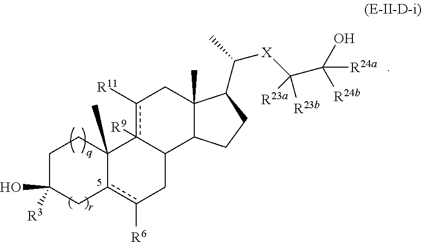

E-147. The compound according to embodiment E-146, wherein the compound of formula (E-II-D-i) is a compound of formula (E-II-D-i-1):

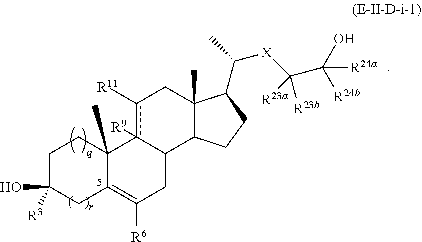

E-148. The compound according to embodiment E-147, wherein the compound of formula (E-II-D-i-1) is a compound of formula (E-II-D-i-1a):

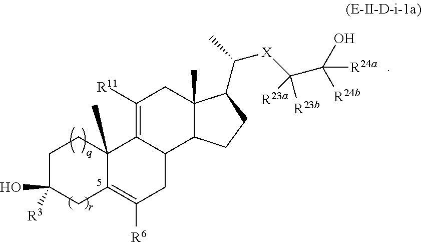

E-149. The compound according to embodiment E-147, wherein the compound of formula (E-II-D-i-1) is a compound of formula (E-II-D-i-1b):

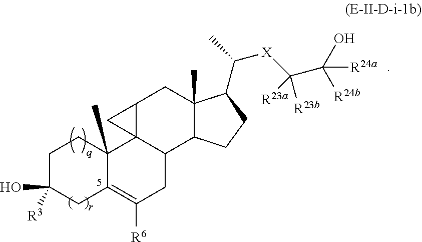

E-150. The compound according to embodiment E-146, wherein the compound of formula (E-II-D-i) is a compound of formula (E-II-D-i-2):

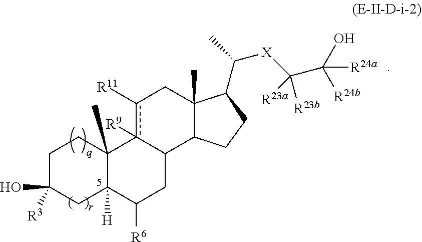

E-151. The compound according to embodiment E-150, wherein the compound of formula (E-II-D-i-2) is a compound of formula (E-II-D-i-2a):

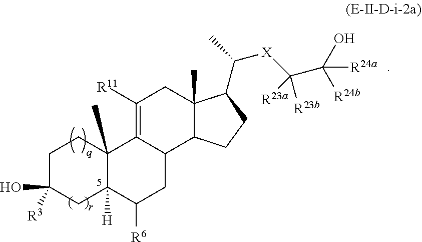

E-152. The compound according to embodiment E-150, wherein the compound of formula (E-II-D-i-2) is a compound of formula (E-II-D-i-2b):

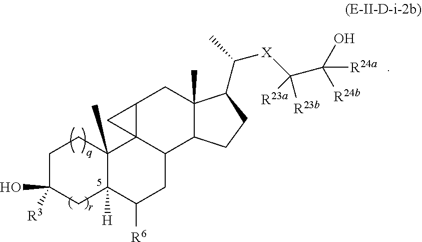

E-153. The compound according to embodiment E-141, wherein the compound of formula (E-II) is a compound of formula (E-II-E):

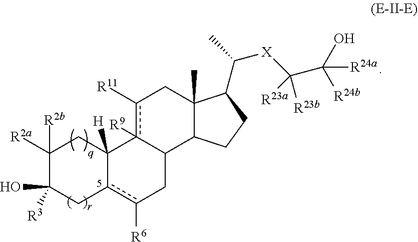

E-154. The compound according to embodiment E-153, wherein the compound of formula (E-II-E) is a compound of formula (E-II-E-i):

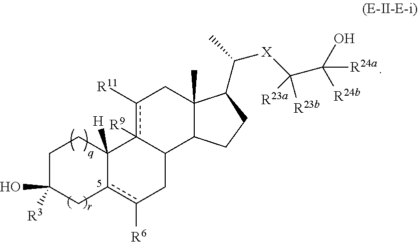

E-155. The compound according to embodiment E-154, wherein the compound of formula (E-II-E-i) is a compound of formula (E-II-E-i-1):

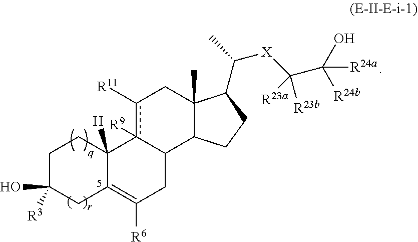

E-156. The compound according to embodiment E-155, wherein the compound of formula (E-II-E-i-1) is a compound of formula (E-II-E-i-1a):

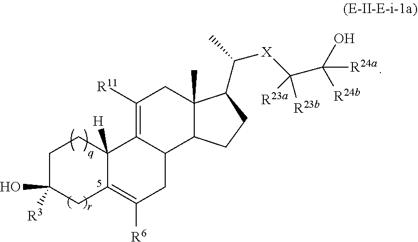

E-157. The compound according to embodiment E-155, wherein the compound of formula (E-II-E-i-1) is a compound of formula (E-II-E-i-1b):

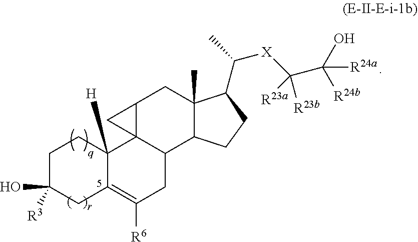

E-158. The compound according to embodiment E-154, wherein the compound of formula (E-II-E-i) is a compound of formula (E-II-E-i-2):

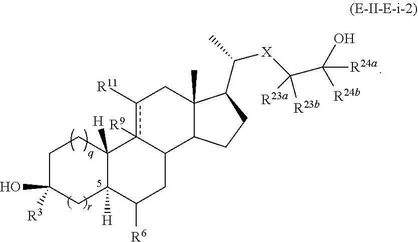

E-159. The compound according to embodiment E-158, wherein the compound of formula (E-II-E-i-2) is a compound of formula (E-II-E-i-2a):

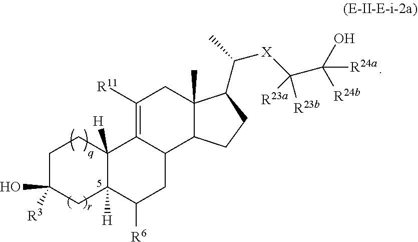

E-160. The compound according to embodiment E-158, wherein the compound of formula (E-II-E-i-2) is a compound of formula (E-II-E-i-2b):

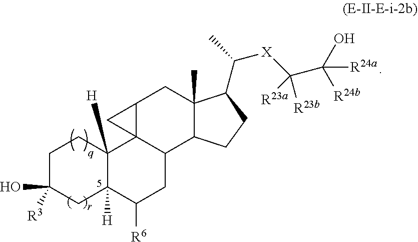

E-161. The compound according to embodiment E-121, wherein the compound of formula (E-I) is a compound of formula (E-III):

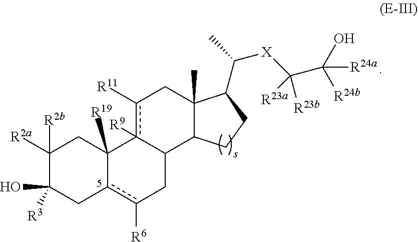

E-162. The compound according to embodiment E-161, wherein the compound of formula (E-III) is a compound of formula (E-III-A):

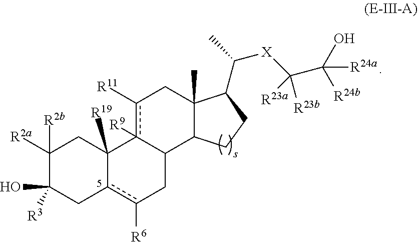

E-163. The compound according to embodiment E-161, wherein the compound of formula (E-III) is a compound of formula (E-III-B):

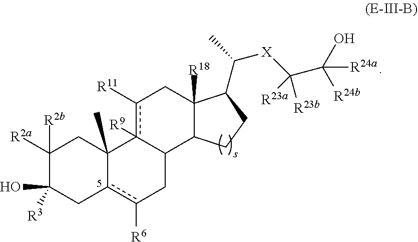

E-164. The compound according to embodiment E-161, wherein the compound of formula (E-III) is a compound of formula (E-III-C):

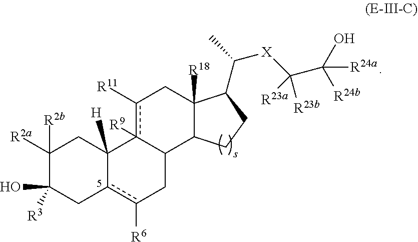

E-165. The compound according to embodiment E-161, wherein the compound of formula (E-III) is a compound of formula (E-III-D):

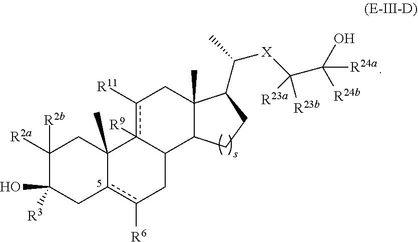

E-166. The compound according to embodiment E-165, wherein the compound of formula (E-III-D) is a compound of formula (E-III-D-i):

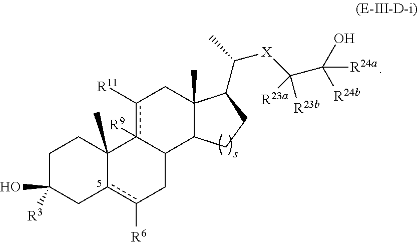

E-167. The compound according to embodiment E-166, wherein the compound of formula (E-III-D-i) is a compound of formula (E-III-D-i-1):

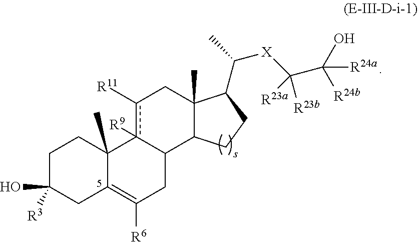

E-168. The compound according to embodiment E-167, wherein the compound of formula (E-III-D-i-1) is a compound of formula (E-III-D-i-1a):

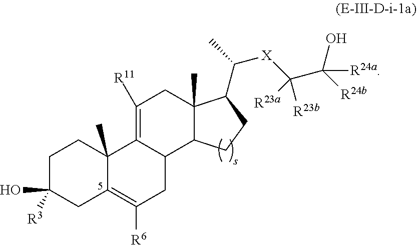

E-169. The compound according to embodiment E-167, wherein the compound of formula (E-III-D-i-1) is a compound of formula (E-III-D-i-1b):

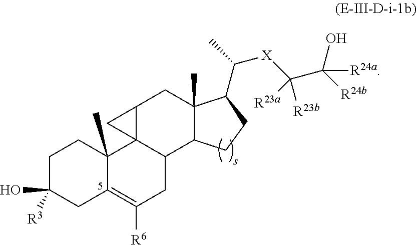

E-170. The compound according to embodiment E-166, wherein the compound of formula (E-III-D-i) is a compound of formula (E-III-D-i-2):

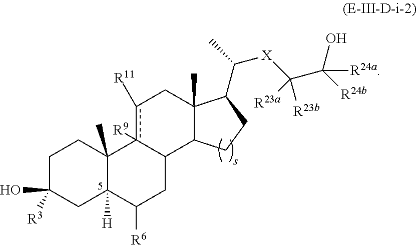

E-171. The compound according to embodiment E-170, wherein the compound of formula (E-III-D-i-2) is a compound of formula (E-III-D-i-2a):

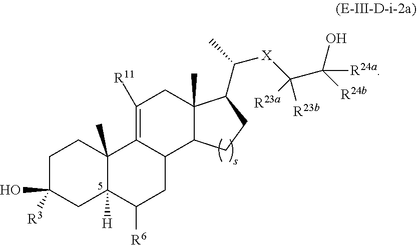

E-172. The compound according to embodiment E-170, wherein the compound of formula (E-III-D-i-2) is a compound of formula (E-III-D-i-2b):

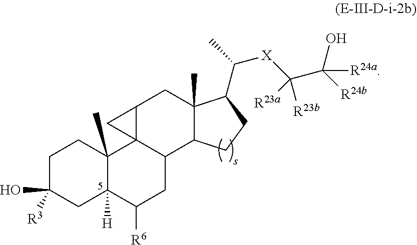

E-173. The compound according to embodiment E-161, wherein the compound of formula (E-III) is a compound of formula (E-III-E):

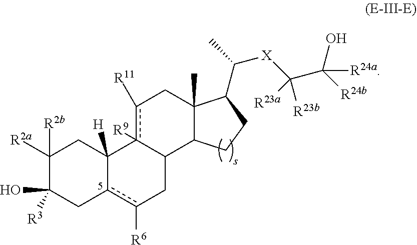

E-174. The compound according to embodiment E-173, wherein the compound of formula (E-III-E) is a compound of formula (E-III-E-i):

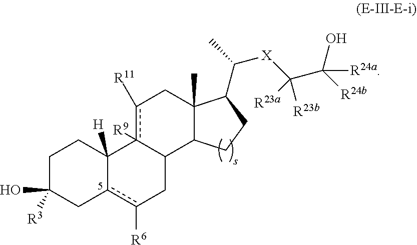

E-175. The compound according to embodiment E-174, wherein the compound of formula (E-III-E-i) is a compound of formula (E-III-E-i-1):

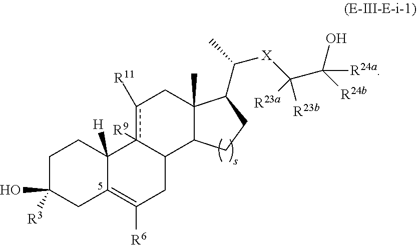

E-176. The compound according to embodiment E-175, wherein the compound of formula (E-III-E-i-1) is a compound of formula (E-III-E-i-1a):

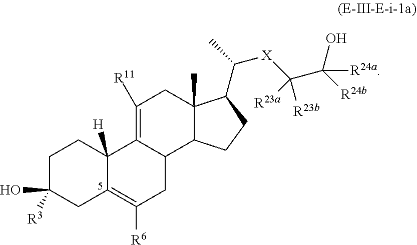

E-177. The compound according to embodiment E-175, wherein the compound of formula (E-III-E-i-1) is a compound of formula (E-III-E-i-1b):

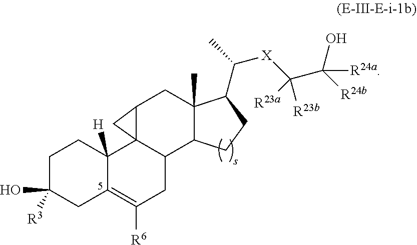

E-178. The compound according to embodiment E-174, wherein the compound of formula (E-III-E-i) is a compound of formula (E-III-E-i-2):

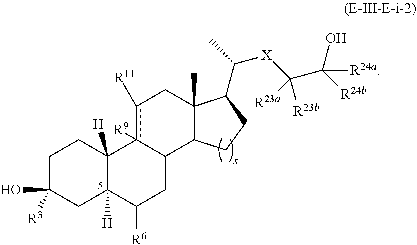

E-179. The compound according to embodiment E-178, wherein the compound of formula (E-III-E-i-2) is a compound of formula (E-III-E-i-2a):

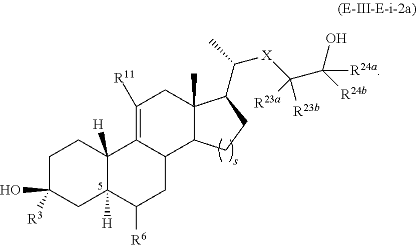

E-180. The compound according to embodiment E-178, wherein the compound of formula (E-III-E-i-2) is a compound of formula (E-III-E-i-2b):

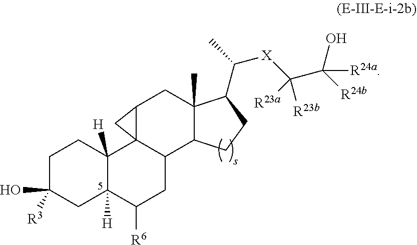

E-181. The compound according to embodiment E-121, wherein the compound of formula (E-I) is a compound of formula (E-IV):

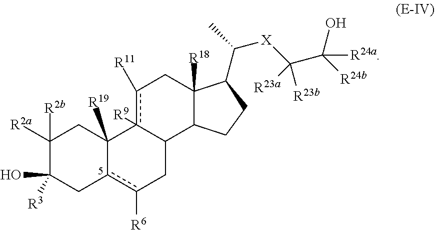

E-182. The compound according to embodiment E-181, wherein the compound of formula (E-IV) is a compound of formula (E-IV-A):

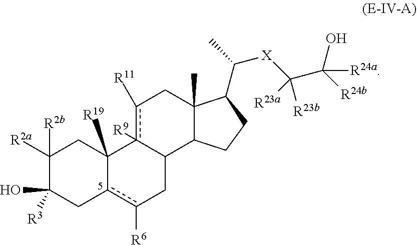

E-183. The compound according to embodiment E-181, wherein the compound of formula (E-IV) is a compound of formula (E-IV-B):

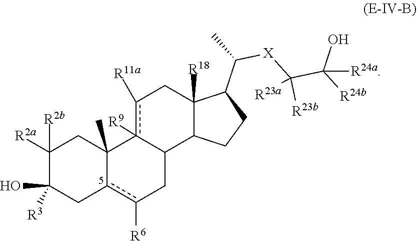

E-184. The compound according to embodiment E-181, wherein the compound of formula (E-IV) is a compound of formula (E-IV-C):

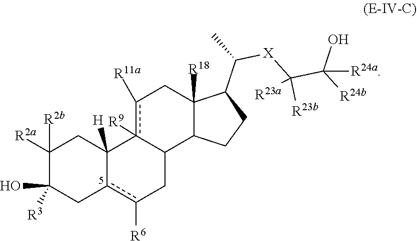

E-185. The compound according to embodiment E-181, wherein the compound of formula (E-IV) is a compound of formula (E-IV-D):

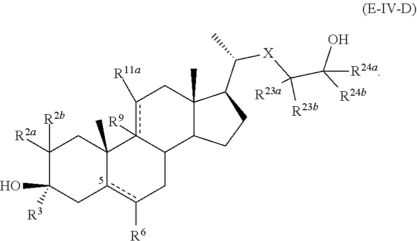

E-186. The compound according to embodiment E-185, wherein the compound of formula (E-IV-D) is a compound of formula (E-IV-D-i):

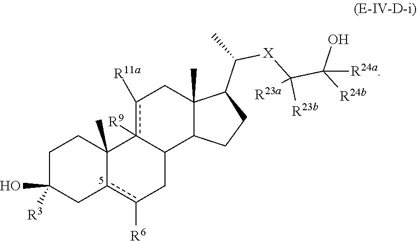

E-187. The compound according to embodiment E-186, wherein the compound of formula (E-IV-D-i) is a compound of formula (E-IV-D-i-1):

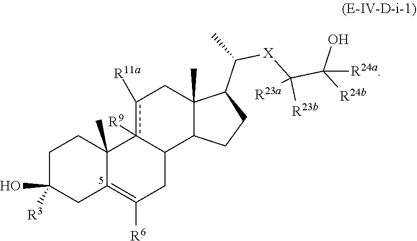

E-188. The compound according to embodiment E-187, wherein the compound of formula (E-IV-D-i-1) is a compound of formula (E-IV-D-i-1a):

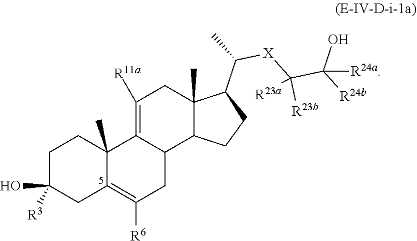

E-189. The compound according to embodiment E-187, wherein the compound of formula (E-IV-D-i-1) is a compound of formula (E-IV-D-i-1b):

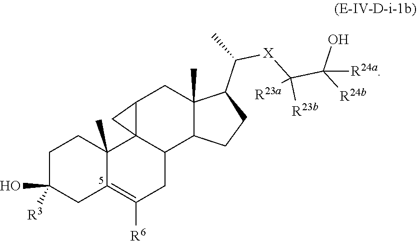

E-190. The compound according to embodiment E-186, wherein the compound of formula (E-IV-D-i) is a compound of formula (E-IV-D-i-2):

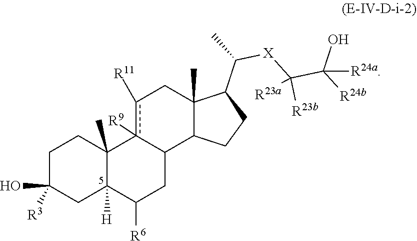

E-191. The compound according to embodiment E-190, wherein the compound of formula (E-IV-D-i-2) is a compound of formula (E-IV-D-i-2a):

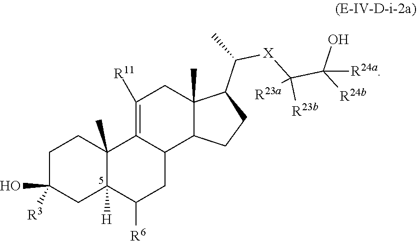

E-192. The compound according to embodiment E-190, wherein the compound of formula (E-IV-D-i-2) is a compound of formula (E-IV-D-i-2b):

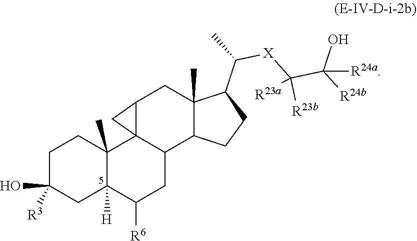

E-193. The compound according to embodiment E-181, wherein the compound of formula (E-IV) is a compound of formula (E-IV-E):

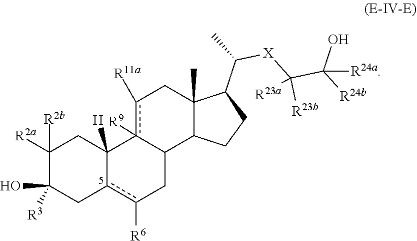

E-194. The compound according to embodiment E-193, wherein the compound of formula (E-IV-E) is a compound of formula (E-IV-E-i):

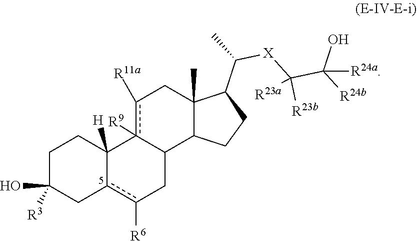

E-195. The compound according to embodiment E-194, wherein the compound of formula (E-IV-E-i) is a compound of formula (E-IV-E-i-1):

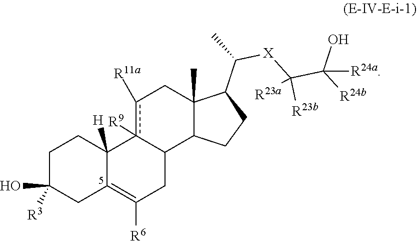

E-196. The compound according to embodiment E-195, wherein the compound of formula (E-IV-E-i-1) is a compound of formula (E-IV-E-i-1a):

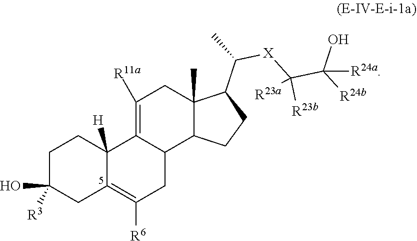

E-197. The compound according to embodiment E-195, wherein the compound of formula (E-IV-E-i-1) is a compound of formula (E-IV-E-i-1b):

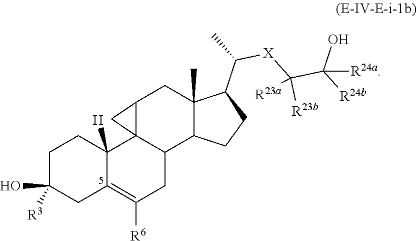

E-198. The compound according to embodiment E-194, wherein the compound of formula (E-IV-E-i) is a compound of formula (E-IV-E-i-2):

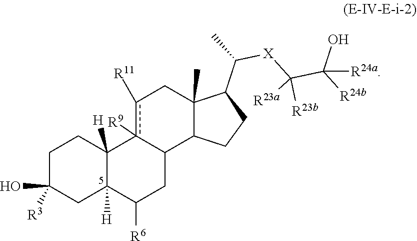

E-199. The compound according to embodiment E-198, wherein the compound of formula (E-IV-E-i-2) is a compound of formula (E-IV-E-i-2a):

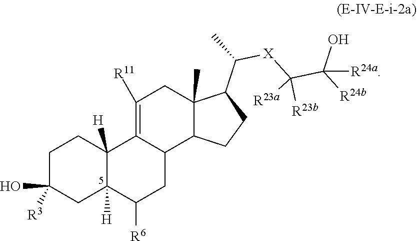

E-200. The compound according to embodiment E-198, wherein the compound of formula (E-IV-E-i-2) is a compound of formula (E-IV-E-i-2b):

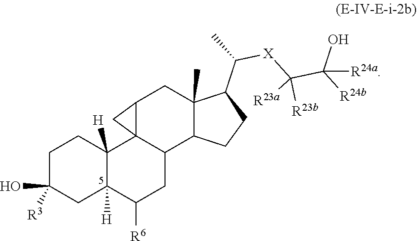

E-201. The compound according to any one of embodiments E-121 to E-200, wherein R³ is unsubstituted C₁₋₆alkyl.

E-202. The compound according to embodiment E-201, wherein R³ is —CH₃, —CH₂CH₃, or —CH₂CH₂CH₃.

E-203. The compound according to embodiment E-202, wherein R³ is —CH₂CH₃.

E-204. The compound according to any one of embodiments E-121 to E-200, wherein R⁶ is hydrogen.

E-205. The compound according to any one of embodiments E-121 to E-127, E-130, E-133 to E-135, E-138, E-141 to E-147, E-150, E-153 to E-156, E-158, E-161 to E-167, E-170, E-173 to E-175, E-178, E-181 to E-187, E-190, E-193 to E-195, and E-198, wherein the bond between C9 and C11 is a double bond, R⁹ is absent and R¹¹ is selected from the group consisting of hydrogen, halogen, substituted or unsubstituted C₁₋₆alkyl, and substituted or unsubstituted C₁₋₆alkoxy.

E-206. The compound according to embodiment E-205, wherein R¹¹ is hydrogen.

E-207. The compound according to any one of embodiments E-121 to E-127, E-130, E-133 to E-135, E-138, E-141 to E-147, E-150, E-153 to E-155, E-158, E-161 to E-167, E-170, E-173 to E-175, E-178, E-181 to E-187, E-190, E-193 to E-195, and E-198, wherein the bond between C9 and C11 is a single bond, R⁹ is hydrogen and R¹¹ is selected from the group consisting of halogen or substituted or unsubstituted C₁₋₆alkyl.

E-208. The compound according to any one of embodiments E-121 to E-128, E-130 to E-131, E-133 to E-136, E-138 to E-139, E-141 to E-148, E-150 to E-151, E-153 to E-156, E-158 to E-159, E-161 to E-168, E-170 to E-171, E-173 to E-176, E-178 to E-179, E-181 to E-188, E-190 to E-191, E-193 to E-196, and E-198 to E-199, wherein the bond between C9 and C11 is a double bond, R⁹ is absent and R¹¹ is selected from the group consisting of hydrogen, halogen, substituted or unsubstituted C₁₋₆alkyl, and substituted or unsubstituted C₁₋₆alkoxy.

E-209. The compound according to embodiment E-208, wherein R¹¹ is hydrogen.

E-210. The compound according to any one of embodiments E-121 to E-200, wherein X is —(C(R^{X})₂)ₙ—, n is 1 or 2 and R^{X} is hydrogen.

E-211. The compound according to embodiment E-210, wherein n is 1.

E-212. The compound according to embodiment E-210, wherein n is 2.

E-213. The compound according to any one of embodiments E-121 to E-200, wherein each of R^{23a} and R^{23b} is independently hydrogen.

E-214. The compound according to any one of embodiments E-121 to E-200, wherein each of R^{23a} and R^{23b} is independently fluorine.

E-215. The compound according to any one of embodiments E-121 to E-200, wherein R^{24a} is substituted or unsubstituted C₁₋₆alkyl.

E-216. The compound according to embodiment E-215, wherein R^{24a} is unsubstituted C₁₋₆alkyl.

E-217. The compound according to embodiment E-215, wherein R^{24a} is C₁₋₆alkyl substituted with fluorine.

E-218. The compound according to embodiment E-215, wherein R^{24a} is —CH₃, —CH(CH₃)₂, or —CH₂CF₃.

E-219. The compound according to any one of embodiments E-121 to E-200, wherein R^{24b} is hydrogen, or substituted or unsubstituted C₁₋₆alkyl.

E-220. The compound according to embodiment E-219, wherein R 24b is hydrogen.

E-221. The compound according to embodiment E-219, wherein R^{24b} is unsubstituted C₁₋₆alkyl.

E-222. The compound according to embodiment E-219, wherein R^{24b} is C₁₋₆alkyl substituted with fluorine.

E-223. The compound according to embodiment E-219, wherein R^{24b} is —CH₃, —CH(CH₃)₂, or —CH₂CF₃.

E-224. The compound according to any one of embodiments E-121 to E-200, wherein R^{24a} and R^{24b}, taken together with the carbon atom to which they are attached, form an unsubstituted C₃₋₆carbocyclyl.

E-225. The compound according to embodiment E-224, wherein R^{24a} and R^{24b}, taken together with the carbon atom to which they are attached, form a cyclopropyl or cyclopentyl.

E-226. The compound according to any one of embodiments E-121 to E-200, wherein R^{24a} and R^{24b}, taken together with the carbon atom to which they are attached, form an unsubstituted 3- to 6-membered heterocyclyl.

E-227. The compound according to embodiment E-226, wherein R^{24a} and R^{24b}, taken together with the carbon atom to which they are attached, form a tetrahydrofuranyl.

E-228. The compound according to embodiment E-121, wherein the compound of Formula (E-I) is selected from the group consisting of Compounds E-1-E-16 of Table E-1.

E-229. The compound according to embodiment E-121, wherein the compound of Formula (E-I) is selected from the group consisting of Compounds C-26-C-35 of Table E-1.

E-230. The compound according to embodiment E-121, wherein the compound of Formula (E-I) is selected from the group consisting of Compounds E-3, E-4, E-6, E-9, E-11, E-12 or pharmaceutically acceptable salts thereof.

E-231. A pharmaceutical composition comprising a compound according to any one of embodiments E-121 to E-230, and a pharmaceutically acceptable carrier.

E-232. A method for treating a CNS-related condition in a subject, comprising administering to the subject an effective amount of a compound according to any one of embodiments E-121 to E-230, or a pharmaceutical composition according to embodiment E-231.

E-233. The method according to embodiment E-232, wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease) and tinnitus.

E-234. A method of inducing sedation or anesthesia in a subject, comprising administering to the subject an effective amount of a compound according to any one of embodiments E-121 to E-230, or a pharmaceutical composition according to embodiment E-231.

E-235. A compound according to any one of embodiments E-121 to E-230, or a pharmaceutical composition according to embodiment E-231, for use in treating a CNS-related condition in a subject.

E-236. The compound or pharmaceutical composition for use according to embodiment E-235, wherein the wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease) and tinnitus.

E-237. A compound according to any one of embodiments E-121 to E-230, or a pharmaceutical composition according to embodiment E-231, for use inducing sedation or anesthesia in a subject.

E-238. Use of a compound according to any one of embodiments E-121 to E-230, or a pharmaceutical composition according to embodiment E-231, for the manufacture of a medicament for treating a CNS-related condition in a subject.

E-239. The use according to embodiment E-238, wherein the wherein the wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease) and tinnitus.

E-240. Use of a compound according to any one of embodiments E-121 to E-230, or a pharmaceutical composition according to embodiment E-231, for the manufacture of a medicament for inducing sedation or anesthesia in a subject.

F-1. A compound of Formula (F-I):

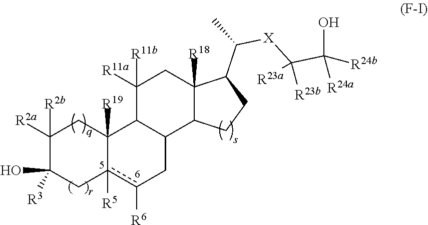

or a pharmaceutically acceptable salt thereof;
wherein:
   each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein R^{A2} is hydrogen or substituted or unsubstituted C₁₋₆alkyl;
   R³ is substituted or unsubstituted C₁₋₆alkyl, substituted or unsubstituted C₂₋₆ alkenyl, substituted or unsubstituted C₂₋₆alkynyl, or substituted or unsubstituted C₃₋₁₀carbocyclyl;
   R⁵ is absent, hydrogen, substituted or unsubstituted C₁₋₆alkyl, or halogen;
   R⁶ is hydrogen, substituted or unsubstituted C₁₋₆alkyl, or halogen;
   R^{11a} is hydrogen or —OR^{A11}, wherein R^{A11} is hydrogen or substituted or unsubstituted C₁₋₆alkyl, and R^{11b} is hydrogen; or R^{11a} and R^{11b} are joined to form an oxo (═O) group;
   R¹⁸ is hydrogen, unsubstituted C₂₋₆alkyl, substituted C₁₋₆alkyl, or OR^{A18}, wherein each instance of R^{A18} is independently substituted or unsubstituted C₁₋₆alkyl;
R¹⁹ is hydrogen, substituted or unsubstituted C₁₋₆alkyl, or OR^{A19}, each instance of R^{A19} is independently substituted or unsubstituted C₁₋₆alkyl;
X is —(C(R^{X})₂)ₙ— or —O—, wherein R^{X} is hydrogen or fluorine, or one R^{X} group and R^{23b} are joined to form a double bond;
   n is 1, 2 or 3;
   each of R^{23a} and R^{23b} is independently hydrogen or fluorine;
   R^{24a} is selected from the group consisting of substituted or unsubstituted C₁₋₆ alkyl, substituted or unsubstituted C₂₋₆alkenyl, substituted or unsubstituted C₂₋₆ alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl; and
   R^{24b} is hydrogen or substituted or unsubstituted C₁₋₆alkyl;
   or R^{24a} and R^{24b}, taken together with the carbon atom to which they are attached, form a substituted or unsubstituted C₃₋₆carbocyclyl, or a substituted or unsubstituted 3- to 10-membered heterocyclyl;
   q is 0, 1 or 2;
   r is 1 or 2;
   s is 1 or 2; and
   [symbol] represents a single or double bond, provided if a double bond is present between C5 and C6, R⁵ is absent.

F-1A. A compound of Formula (F-IA):

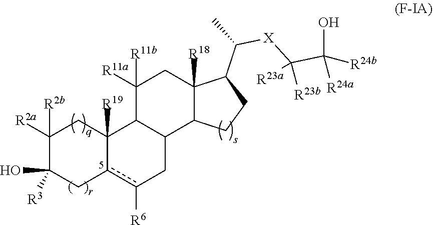

or a pharmaceutically acceptable salt thereof;
wherein:
   each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein R^{A2} is hydrogen or substituted or unsubstituted C₁₋₆alkyl;
   R³ is substituted or unsubstituted C₁₋₆alkyl, substituted or unsubstituted C₂₋₆alkenyl, substituted or unsubstituted C₂₋₆alkynyl, or substituted or unsubstituted C₃₋₁₀carbocyclyl;
   R⁶ is hydrogen, substituted or unsubstituted C₁₋₆alkyl, or halogen;
   R^{11a} is hydrogen or —OR^{A11}, wherein R^{A11} is hydrogen or substituted or unsubstituted C₁₋₆alkyl, and R^{11b} is hydrogen; or R^{11a} and R^{11b} are joined to form an oxo (═O) group;
   R¹⁸ is hydrogen, unsubstituted C₂₋₆alkyl, substituted C₁₋₆alkyl, or OR^{A18}, wherein each instance of R^{A18} is independently substituted or unsubstituted C₁₋₆alkyl;
   R¹⁹ is hydrogen, substituted or unsubstituted C₁₋₆alkyl, or OR^{A19}, wherein each instance of R^{A19} is independently substituted or unsubstituted C₁₋₆alkyl;
   X is —(C(R^{X})₂)ₙ— or —O—, wherein R^{X} is hydrogen or fluorine, or one R^{X} group and R^{23b} are joined to form a double bond;
   n is 1, 2 or 3;
   each of R^{23a} and R^{23b} is independently hydrogen or fluorine;
   R^{24a} is selected from the group consisting of substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl; and
   R^{24b} is hydrogen or substituted or unsubstituted C₁₋₆alkyl;
   or R^{24a} and R^{24b}, taken together with the carbon atom to which they are attached, form a substituted or unsubstituted C₃₋₆carbocyclyl, or a substituted or unsubstituted 3- to 10-membered heterocyclyl;
   q is 0, 1 or 2;
   r is 1 or 2;
   s is 1 or 2; and
   [symbol] represents a single or double bond, provided if a single bond is present, then the hydrogen at C5 is in the alpha configuration.

F-2. The compound or pharmaceutically acceptable salt thereof according to embodiment F-1, wherein the compound of Formula (F-I) is a compound of Formula (F-Ia):

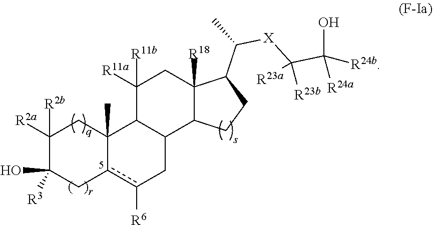

F-3. The compound or pharmaceutically acceptable salt thereof according to embodiment F-2, wherein the compound of Formula (F-Ia) is a compound of Formula (F-Ia-i):

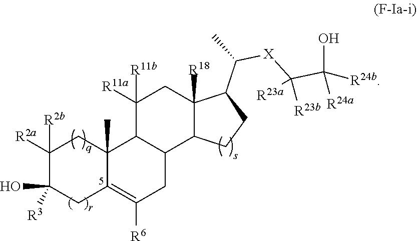

F-4. The compound or pharmaceutically acceptable salt thereof according to embodiment F-3, wherein the compound of Formula (F-Ia-i) is a compound of Formula (F-Ia-i-1) or Formula (F-Ia-i-2):

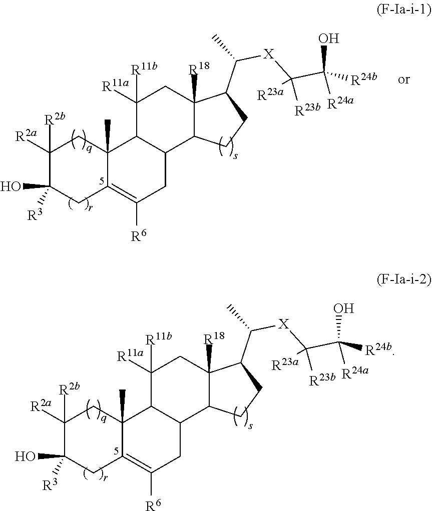

F-5. The compound or pharmaceutically acceptable salt thereof according to embodiment F-2, wherein the compound of Formula (F-Ia) is a compound of Formula (F-Ia-ii):

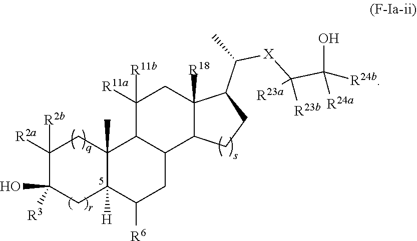

F-6. The compound or pharmaceutically acceptable salt thereof according to embodiment F-5, wherein the compound of Formula (F-Ia-ii) is a compound of Formula (F-Ia-ii-1) or Formula (F-Ia-ii-2):

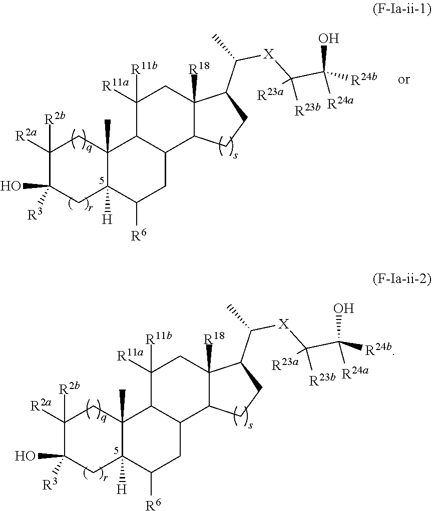

F-7. The compound or pharmaceutically acceptable salt thereof according to embodiment F-1, wherein the compound of Formula (F-I) is a compound of Formula (F-Ib)

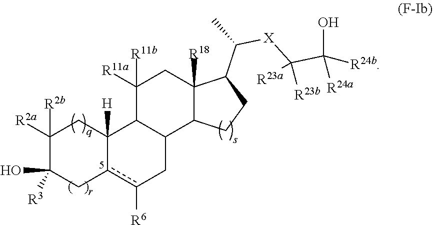

F-8. The compound or pharmaceutically acceptable salt thereof according to embodiment F-7, wherein the compound of Formula (F-Ib) is a compound of Formula (F-Ib-i):

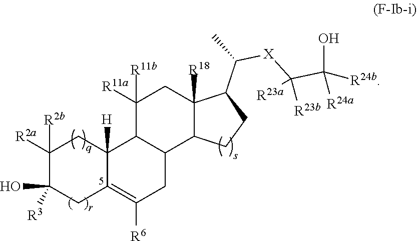

F-9. The compound or pharmaceutically acceptable salt thereof according to embodiment F-8, wherein the compound of Formula (F-Ib-i) is a compound of Formula (F-Ib-i-1) or Formula (F-Ib-i-2):

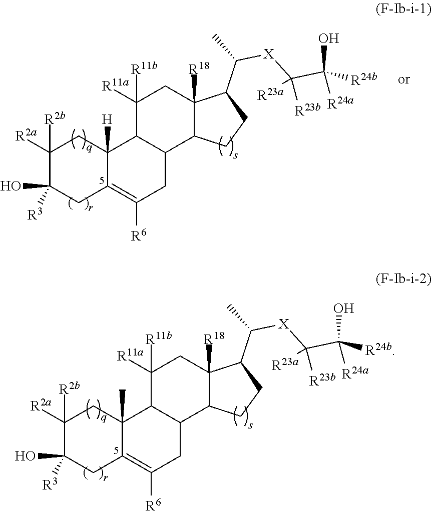

F-10. The compound or pharmaceutically acceptable salt thereof according to embodiment F-7, wherein the compound of Formula (F-Ib) is a compound of Formula (F-Ib-ii):

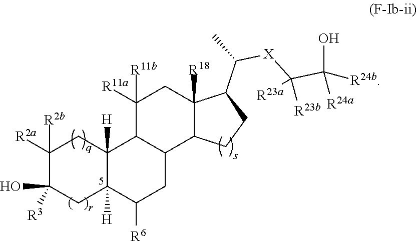

F-11. The compound or pharmaceutically acceptable salt thereof according to embodiment F-10, wherein the compound of Formula (F-Ib-ii) is a compound of Formula (F-Ib-ii-1) or Formula (F-Ib-ii-2):

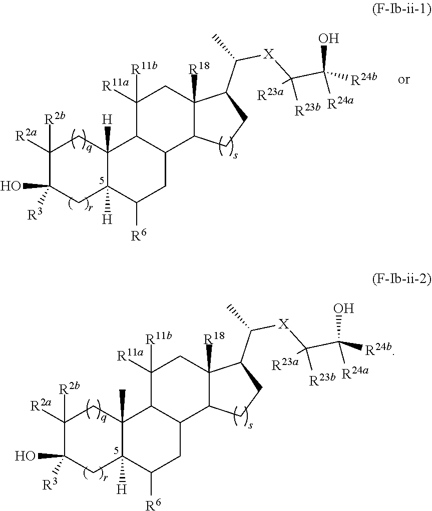

F-12. The compound or pharmaceutically acceptable salt thereof according to embodiment F-1, wherein the compound of Formula (F-I) is a compound of Formula (F-II):

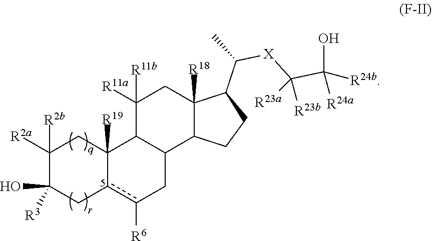

F-13. The compound or pharmaceutically acceptable salt thereof according to embodiment F-12, wherein the compound of Formula (F-II) is a compound of Formula (F-IIa):

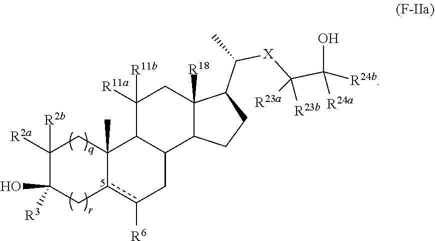

F-14. The compound or pharmaceutically acceptable salt thereof according to embodiment F-13, wherein the compound of Formula (F-IIa) is a compound of Formula (F-IIa-i):

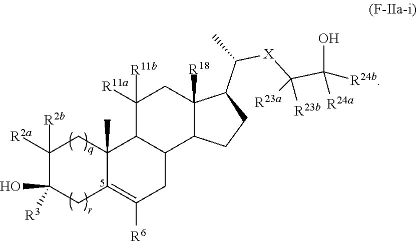

F-15. The compound or pharmaceutically acceptable salt thereof according to embodiment F-14, wherein the compound of Formula (F-IIa-i) is a compound of Formula (F-IIa-i-1) or Formula (F-IIa-i-2):

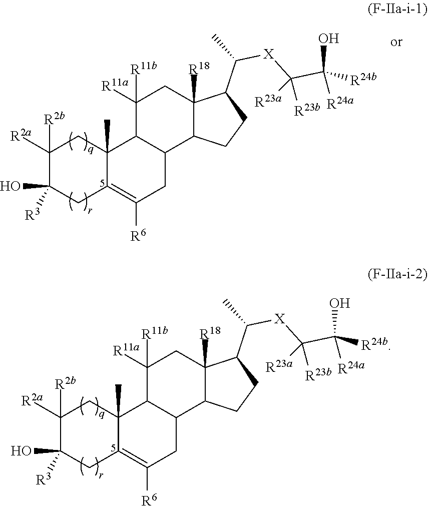

F-16. The compound or pharmaceutically acceptable salt thereof according to embodiment F-13, wherein the compound of Formula (F-IIa) is a compound of Formula (F-IIa-ii):

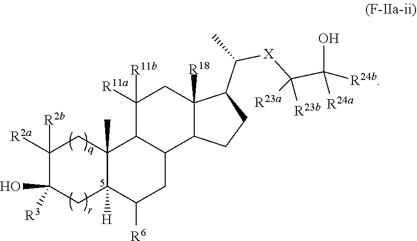

F-17. The compound or pharmaceutically acceptable salt thereof according to embodiment F-16, wherein the compound of Formula (F-IIa-ii) is a compound of Formula (F-IIa-ii-1) or Formula (F-IIa-ii-2):

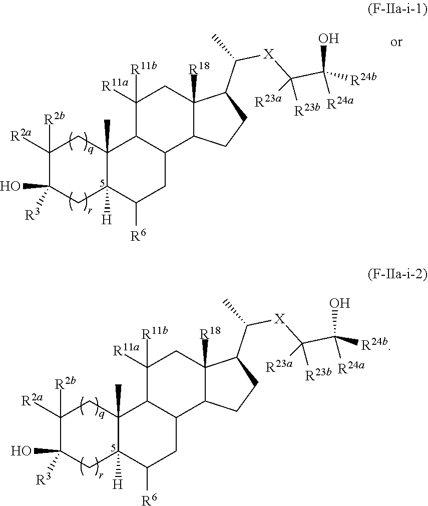

F-18. The compound or pharmaceutically acceptable salt thereof according to embodiment F-12, wherein the compound of Formula (F-II) is a compound of Formula (F-IIb):

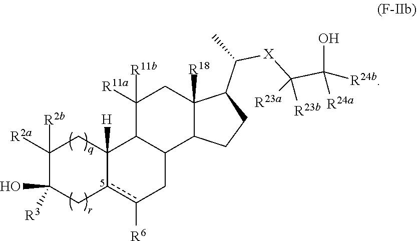

F-19. The compound or pharmaceutically acceptable salt thereof according to embodiment F-18, wherein the compound of Formula (F-IIb) is a compound of Formula (F-IIb-i):

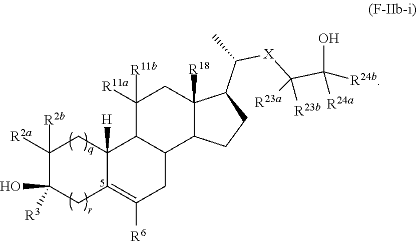

F-20. The compound or pharmaceutically acceptable salt thereof according to embodiment F-19, wherein the compound of Formula (F-IIb-i) is a compound of Formula (F-IIb-i-1) or Formula (F-IIb-i-2):

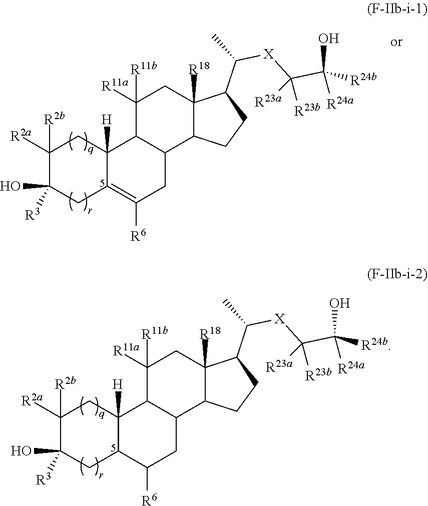

F-21. The compound or pharmaceutically acceptable salt thereof according to embodiment F-18, wherein the compound of Formula (F-IIb) is a compound of Formula (F-IIb-ii):

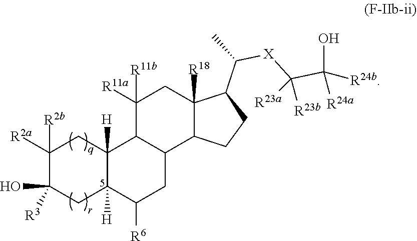

F-22. The compound or pharmaceutically acceptable salt thereof according to embodiment F-21, wherein the compound of Formula (F-IIb-ii) is a compound of Formula (F-IIb-ii-1) or Formula (F-IIb-ii-2):

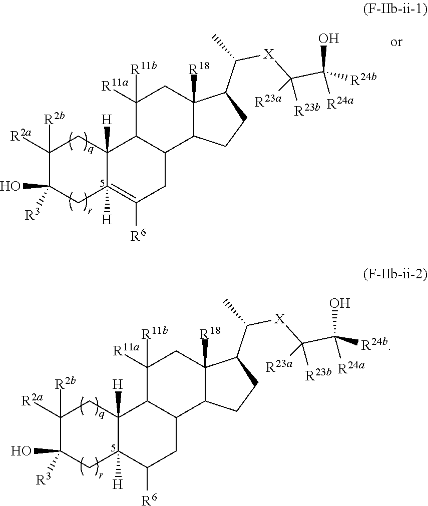

F-23. The compound or pharmaceutically acceptable salt thereof according to embodiment F-1, wherein the compound of Formula (F-I) is a compound of Formula (F-III):

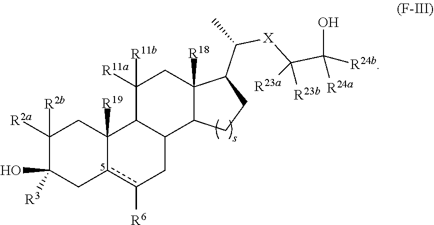

F-24. The compound or pharmaceutically acceptable salt thereof according to embodiment F-23, wherein the compound of Formula (F-III) is a compound of Formula (F-IIIa):

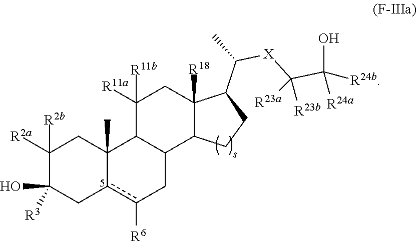

F-25. The compound or pharmaceutically acceptable salt thereof according to embodiment F-24, wherein the compound of Formula (F-IIIa) is a compound of Formula (F-IIIa-i):

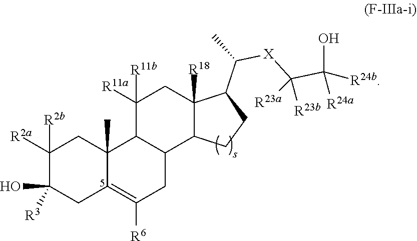

F-26. The compound or pharmaceutically acceptable salt thereof according to embodiment F-25, wherein the compound of Formula (F-IIIa-i) is a compound of Formula (F-IIIa-i-1) or Formula (F-IIIa-i-2):

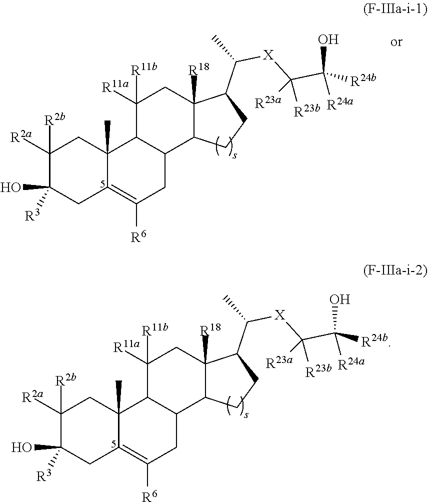

F-27. The compound or pharmaceutically acceptable salt thereof according to embodiment F-24, wherein the compound of Formula (F-IIIa) is a compound of Formula (F-IIIa-ii):

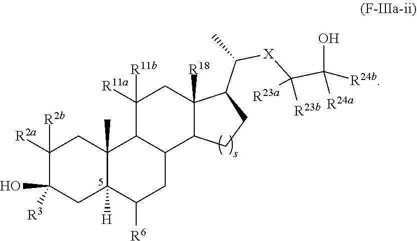

F-28. The compound or pharmaceutically acceptable salt thereof according to embodiment F-27, wherein the compound of Formula (F-IIIa-ii) is a compound of Formula (F-IIIa-ii-1) or Formula (F-IIIa-ii-2):

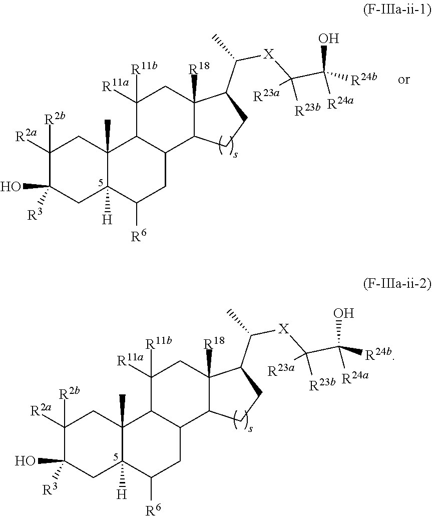

F-29. The compound or pharmaceutically acceptable salt thereof according to embodiment F-23, wherein the compound of Formula (F-III) is a compound of Formula (F-IIIb):

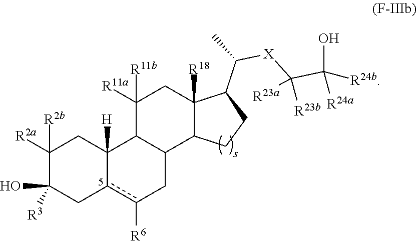

F-30. The compound or pharmaceutically acceptable salt thereof according to embodiment F-29, wherein the compound of Formula (F-IIIb) is a compound of Formula (F-IIIb-i):

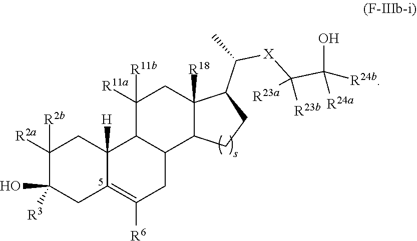

F-31. The compound or pharmaceutically acceptable salt thereof according to embodiment F-30, wherein the compound of Formula (F-IIIb-i) is a compound of Formula (F-IIIb-i-1) or Formula (F-IIIb-i-2):

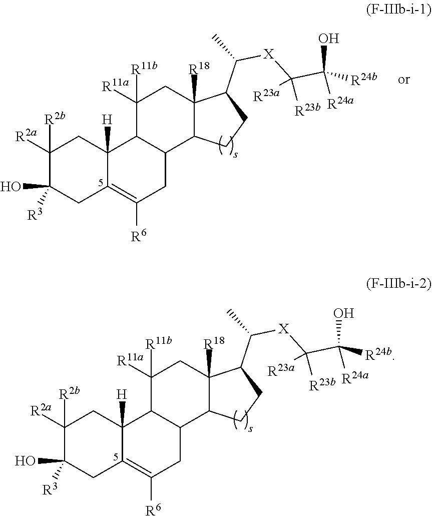

F-32. The compound or pharmaceutically acceptable salt thereof according to embodiment F-29, wherein the compound of Formula (F-IIIb) is a compound of Formula (F-IIIb-ii):

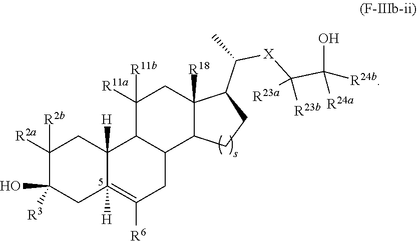

F-33. The compound or pharmaceutically acceptable salt thereof according to embodiment F-32, wherein the compound of Formula (F-IIIb-ii) is a compound of Formula (F-IIIb-ii-1) or Formula (F-IIIb-ii-2):

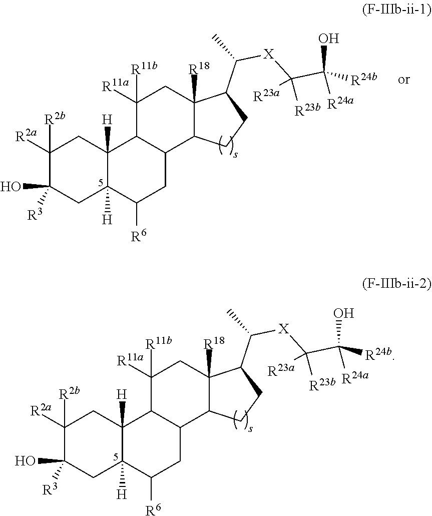

F-34. The compound or pharmaceutically acceptable salt thereof according to embodiment F-1, wherein the compound of Formula (F-I) is a compound of Formula (F-IV):

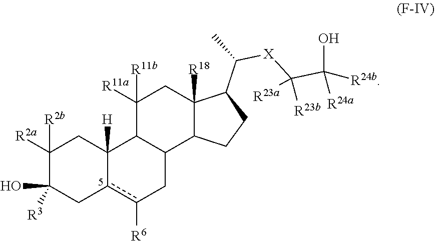

F-35. The compound or pharmaceutically acceptable salt thereof according to embodiment F-34, wherein the compound of Formula (F-IV) is a compound of Formula (F-IVa):

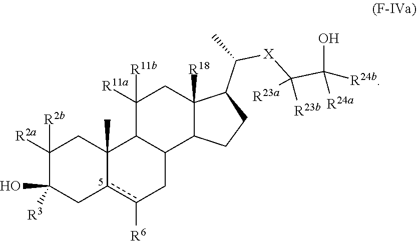

F-36. The compound or pharmaceutically acceptable salt thereof according to embodiment F-35, wherein the compound of Formula (F-IVa) is a compound of Formula (F-IVa-i):

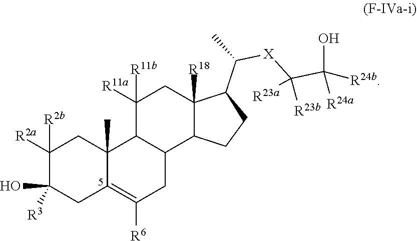

F-37. The compound or pharmaceutically acceptable salt thereof according to embodiment F-36, wherein the compound of Formula (F-IVa-i) is a compound of Formula (F-IVa-i-1) or Formula (F-IVa-i-2):

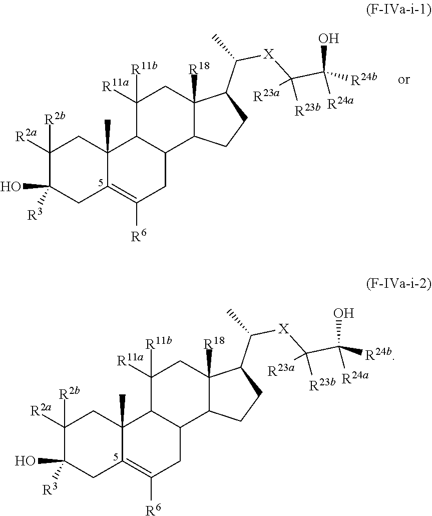

F-38. The compound or pharmaceutically acceptable salt thereof according to embodiment F-35, wherein the compound of Formula (F-IVa) is a compound of Formula (F-IVa-ii):

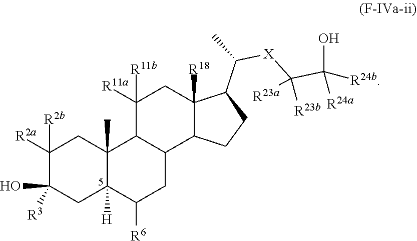

F-39. The compound or pharmaceutically acceptable salt thereof according to embodiment F-38, wherein the compound of Formula (F-IVa-ii) is a compound of Formula (F-IVa-ii-1) or Formula (F-IVa-ii-2):

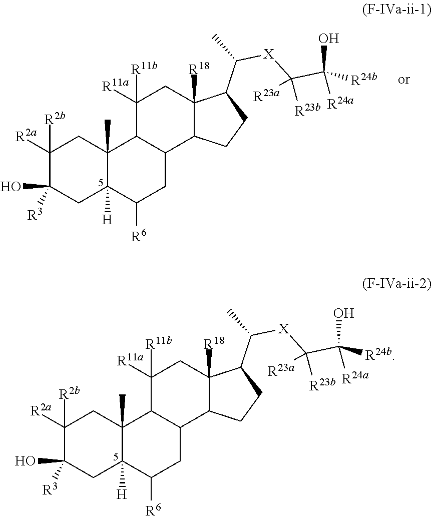

F-40. The compound or pharmaceutically acceptable salt thereof according to embodiment F-34, wherein the compound of Formula (F-IV) is a compound of Formula (F-IVb):

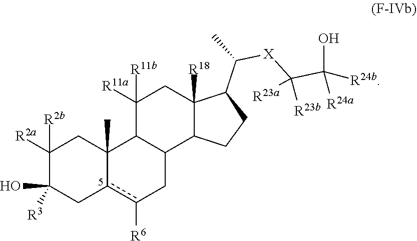

F-41. The compound or pharmaceutically acceptable salt thereof according to embodiment F-40, wherein the compound of Formula (F-IVb) is a compound of Formula (F-IVb-i):

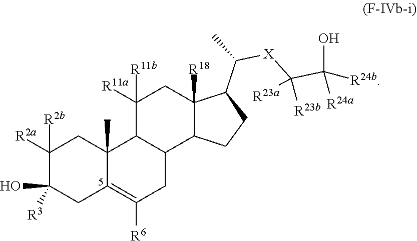

F-42. The compound or pharmaceutically acceptable salt thereof according to embodiment F-41, wherein the compound of Formula (F-IVb-i) is a compound of Formula (F-IVb-i-1) or Formula (F-IVb-i-2):

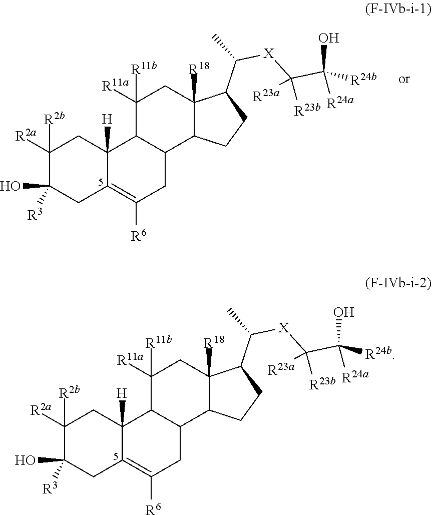

F-43. The compound or pharmaceutically acceptable salt thereof according to embodiment F-40, wherein the compound of Formula (F-IVb) is a compound of Formula (F-IVb-ii):

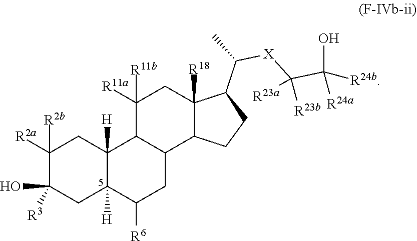

F-44. The compound or pharmaceutically acceptable salt thereof according to embodiment F-43, wherein the compound of Formula (F-IVb-ii) is a compound of Formula (F-IVb-ii-1) or Formula (F-IVb-ii-2):

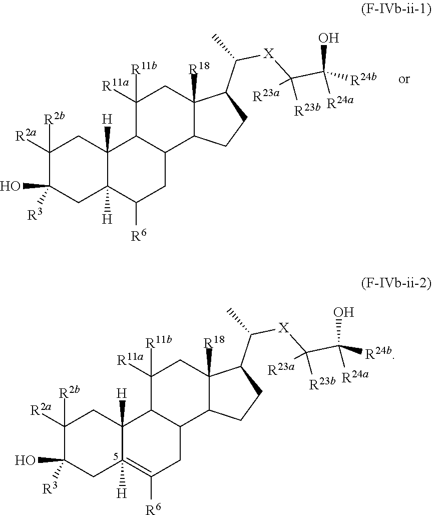

F-45. The compound or pharmaceutically acceptable salt thereof according to embodiment F-1, wherein the compound of Formula (F-I) is a compound of Formula (F-V)

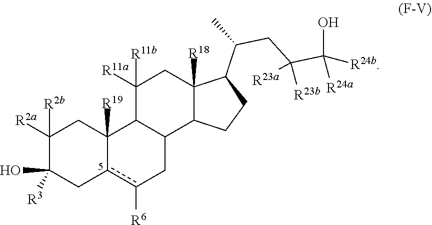

F-46. The compound or pharmaceutically acceptable salt thereof according to embodiment F-45, wherein the compound of Formula (F-V) is a compound of Formula (F-Va):

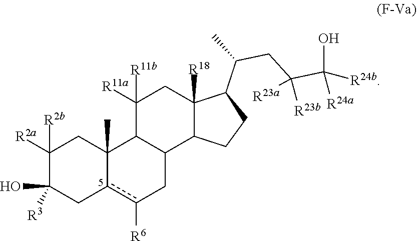

F-47. The compound or pharmaceutically acceptable salt thereof according to embodiment F-46, wherein the compound of Formula (F-Va) is a compound of Formula (F-Va-i):

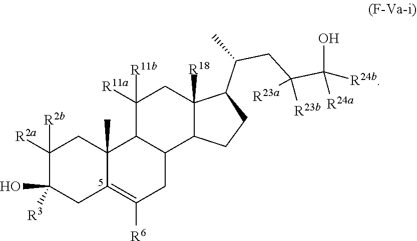

F-48. The compound or pharmaceutically acceptable salt thereof according to embodiment F-47, wherein the compound of Formula (F-Va-i) is a compound of Formula (F-Va-i-1) or Formula (F-Va-i-2):

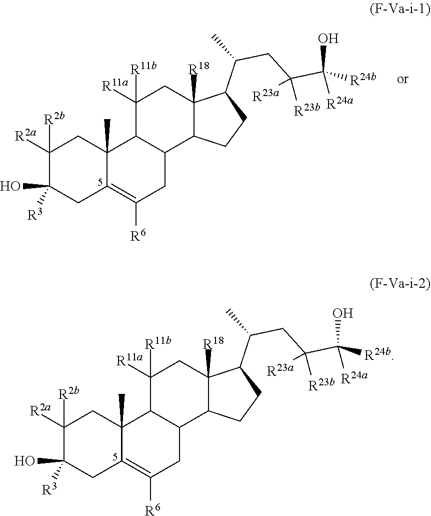

F-49. The compound or pharmaceutically acceptable salt thereof according to embodiment F-46, wherein the compound of Formula (F-Va) is a compound of Formula (F-Va-ii):

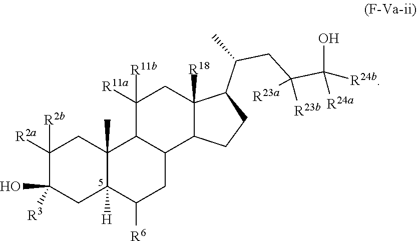

F-50. The compound or pharmaceutically acceptable salt thereof according to embodiment F-49, wherein the compound of Formula (F-Va-ii) is a compound of Formula (F-Va-ii-1) or Formula (F-Va-ii-2):

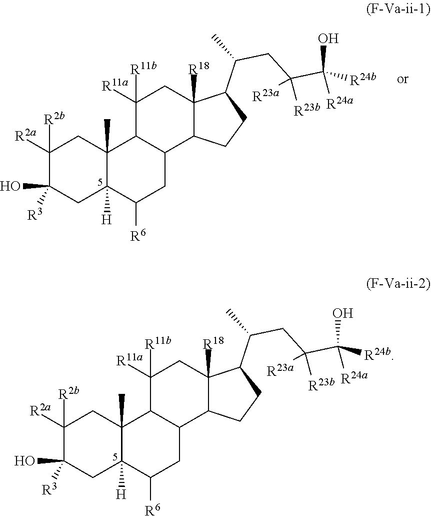

F-51. The compound or pharmaceutically acceptable salt thereof according to embodiment F-45, wherein the compound of Formula (F-V) is a compound of Formula (F-Vb):

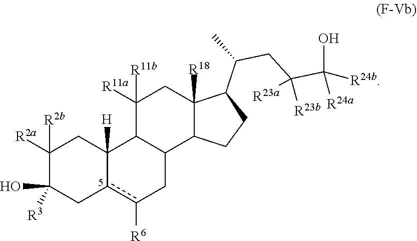

F-52. The compound or pharmaceutically acceptable salt thereof according to embodiment F-51, wherein the compound of Formula (F-Vb) is a compound of Formula (F-Vb-i):

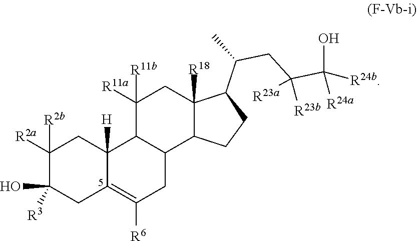

F-53. The compound or pharmaceutically acceptable salt thereof according to embodiment F-52, wherein the compound of Formula (F-Vb-i) is a compound of Formula (F-Vb-i-1) or Formula (F-Vb-i-2):

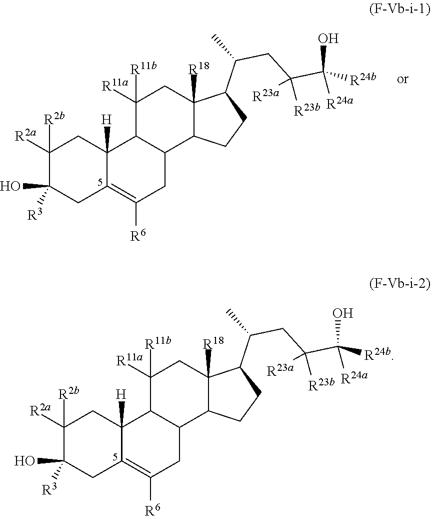

F-54. The compound or pharmaceutically acceptable salt thereof according to embodiment F-51, wherein the compound of Formula (F-Vb) is a compound of Formula (F-Vb-ii):

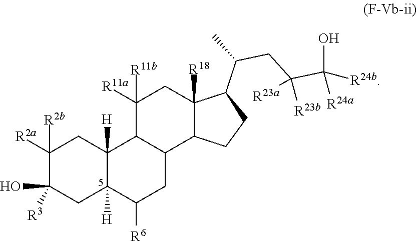

F-55. The compound or pharmaceutically acceptable salt thereof according to embodiment F-54, wherein the compound of Formula (F-Vb-ii) is a compound of Formula (F-Vb-ii-1) or Formula (F-Vb-ii-2):

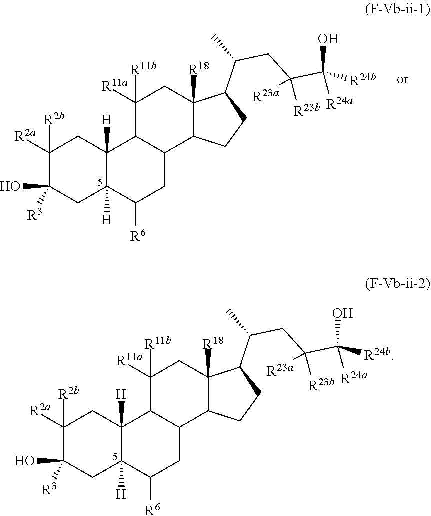

F-56. The compound or pharmaceutically acceptable salt thereof according to any one of embodiments F-1 to F-55, wherein each of R^{2a} and R^{2b} is hydrogen.

F-57. The compound or pharmaceutically acceptable salt thereof according to any one of embodiments F-1 to F-55, wherein R³ is unsubstituted C₁₋₆alkyl.

F-58. The compound or pharmaceutically acceptable salt thereof according to embodiment F-57, wherein R³ is —CH₃, —CH₂CH₃, or —CH₂CH₂CH₃.

F-59. The compound or pharmaceutically acceptable salt thereof according to embodiment F-58, wherein R³ is —CH₃.

F-60. The compound or pharmaceutically acceptable salt thereof according to embodiment F-58, wherein R³ is —CH₂CH₃.

F-61. The compound or pharmaceutically acceptable salt thereof according to any one of embodiments F-1 to F-55, wherein each of R^{11a} and R^{11b} is hydrogen.

F-62. The compound of or pharmaceutically acceptable salt thereof according to embodiments F-1 to F-55, wherein R⁶ is hydrogen.

F-63. The compound or pharmaceutically acceptable salt thereof according to any one of embodiments F-1 to F-55, wherein R¹⁸ is hydrogen, unsubstituted C₂₋₆alkyl, C₁₋₆ alkyl substituted with OR^{A18}, or —OR^{A18}, wherein each instance of R^{A18} is independently unsubstituted C₁₋₆alkyl.

F-64. The compound or pharmaceutically acceptable salt thereof according to embodiment F-63, wherein R¹⁸ is hydrogen, —CH₂OCH₃, —OCH₃, —CH₂CH₃, or —CH(CH₃)₂.

F-65. The compound or pharmaceutically acceptable salt thereof according to embodiment F-64, wherein R¹⁸ is hydrogen.

F-66. The compound or pharmaceutically acceptable salt thereof according to embodiment F-64, wherein R¹⁸ is —CH₂CH₃.

F-67. The compound or pharmaceutically acceptable salt thereof according to any one of embodiments F-1 to F-55, wherein each of R^{23a} and R^{23b} is hydrogen.

F-68. The compound or pharmaceutically acceptable salt thereof according to any one of embodiments F-1 to F-55, wherein at least one of R^{23a} and R^{23b} is fluorine.

F-69. The compound or pharmaceutically acceptable salt thereof according to any one of embodiments F-1 to F-55, wherein each of R^{23a} and R^{23b} is fluorine.

F-70. The compound or pharmaceutically acceptable salt thereof according to any one of embodiments F-1 to F-55, wherein R^{24a} is a non-hydrogen group substituted with fluorine.

F-71. The compound or pharmaceutically acceptable salt thereof according to any one of embodiments F-1 to F-55, wherein R^{24a} is a non-hydrogen group substituted with one or more —OR^{A24} groups, wherein R^{A24} is hydrogen or substituted or unsubstituted C₁₋₆alkyl.

F-72. The compound or pharmaceutically acceptable salt thereof according to any one of embodiments F-1 to F-55, wherein R^{24a} is —CH₂OR^{A24}, —CH₂CH₂OR^{A24}, or —CH₂CH₂CH₂OR^{A24}.

F-73. The compound or pharmaceutically acceptable salt thereof according to any one of embodiments F-1 to F-55, wherein R^{24a} is a substituted or unsubstituted C₁₋₄alkyl, substituted or unsubstituted C₂₋₃alkenyl, substituted or unsubstituted C₂₋₃ alkynyl, or substituted or unsubstituted C₃carbocyclyl.

F-74. The compound or pharmaceutically acceptable salt thereof according to embodiment F-73, wherein R^{24a} is —CF₃.

F-75. The compound or pharmaceutically acceptable salt thereof according to embodiment F-73, wherein R^{24a} is —CH(CH₃)₂.

F-76. The compound or pharmaceutically acceptable salt thereof according to any one of embodiments F-1 to F-55, wherein R^{24b} is hydrogen.

F-77. The compound or pharmaceutically acceptable salt thereof according to any one of embodiments F-1 to F-55, wherein R^{24b} is —CH₃ or —CF₃.

F-78. The compound or pharmaceutically acceptable salt thereof according to any one of embodiments F-1 to F-55, wherein R^{24b} is —CH₃.

F-79. The compound or pharmaceutically acceptable salt thereof according to any one of embodiments F-1 to F-55, wherein R^{24a} is —CH(CH₃)₂ or —CF₃, and R^{24b} is hydrogen.

F-80. The compound or pharmaceutically acceptable salt thereof according to any one of embodiments F-1 to F-55, wherein R^{24a} is a non-hydrogen group substituted with fluorine, and R^{24b} is —CH₃.

F-81. The compound or pharmaceutically acceptable salt thereof according to any one of embodiments F-1 to F-55, wherein R^{24a} is substituted with one or more —OR^{A24} groups, wherein R^{A24} is hydrogen or substituted or unsubstituted C₁₋₆alkyl.

F-82. The compound or pharmaceutically acceptable salt thereof according to any one of embodiments F-1 to F-55, wherein R^{24a} is a substituted or unsubstituted C₁₋₄alkyl, substituted or unsubstituted C₂₋₃alkenyl, substituted or unsubstituted C₂₋₃ alkynyl, or substituted or unsubstituted C₃carbocyclyl, and R^{24b} is —H.

F-83. The compound according to any one of embodiments F-1 to F-55, wherein when R^{24b} is —H, R^{24a} is not C₁₋₆alkyl substituted with CN.

F-84. The compound or pharmaceutically acceptable salt thereof according to any one of embodiments F-1 to F-55, wherein R³ is C₁₋₃ alkyl, R^{24a} is a non-hydrogen group substituted with fluorine, and R^{24b} is —CH₃.

F-85. The compound or pharmaceutically acceptable salt thereof according to any one of embodiments F-1 to F-55, wherein R³ is C₁₋₃ alkyl, R^{24a} is a non-hydrogen group substituted with fluorine, and R^{24b} is hydrogen.

F-86. The compound or pharmaceutically acceptable salt thereof according to any one of embodiments F-1 to F-55, wherein R³ is —CH₃ or —CH₂CH₃, R^{24a} is —CH(CH₃)₂ or —CF₃, and R^{24b} is hydrogen.

F-87. The compound or pharmaceutically acceptable salt thereof according to embodiment F-1, wherein the compound of formula (F-I) is selected from the group consisting of Compounds F-1-F-14 of Table F-1, and pharmaceutically acceptable salts thereof.

F-88. The compound or pharmaceutically acceptable salt thereof according to embodiment F-1, wherein the compound of formula (F-I) is selected from the group consisting of Compounds F1-F12 of Table F-1, and pharmaceutically acceptable salts thereof.

F-89. A pharmaceutical composition comprising a compound or pharmaceutically acceptable salt thereof according to any one of embodiments F-1 to F-88, and a pharmaceutically acceptable carrier.

F-90. A method for treating a CNS-related condition in a subject, comprising administering to the subject an effective amount of a compound or pharmaceutically acceptable salt thereof according to any one of embodiments F-1 to F-88, or a pharmaceutical composition according to embodiment F-89.

F-91. The method according to embodiment F-90, wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease), and tinnitus.

F-92. A method of inducing sedation or anesthesia in a subject, comprising administering to the subject an effective amount of a compound or pharmaceutically acceptable salt thereof according to any one of embodiments F-1 to F-88, or a pharmaceutical composition according to embodiment F-89.

F-93. A compound or pharmaceutically acceptable salt thereof according to any one of embodiments F-1 to F-88, or a pharmaceutical composition according to embodiment F-89, for use in treating a CNS-related condition in a subject.

F-94. The compound or pharmaceutically acceptable salt thereof, or pharmaceutical composition for use according to embodiment F-93, wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease), and tinnitus.

F-95. A compound or pharmaceutically acceptable salt thereof according to any one of embodiments F-1 to F-88, or a pharmaceutical composition according to embodiment F-89, for use in inducing sedation or anesthesia in a subject.

F-96. Use of a compound or pharmaceutically acceptable salt thereof according to any one of embodiments F-1 to F-88, or a pharmaceutical composition according to embodiment F-89, for the manufacture of a medicament for treating a CNS-related condition in a subject.

F-97. The use according to embodiment F-96, wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease) and tinnitus.

F-98. Use of a compound or pharmaceutically acceptable salt thereof according to any one of embodiments F-1 to F-88, or a pharmaceutical composition according to embodiment F-89, for the manufacture of a medicament for inducing sedation or anesthesia in a subject.

F-99. A compound of Formula (F-I):

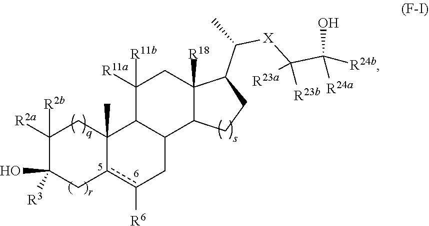

wherein:
   each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein R^{A2} is hydrogen or substituted or unsubstituted C₁₋₆alkyl;
   R³ is substituted or unsubstituted C₁₋₆alkyl, substituted or unsubstituted C₂₋₆ alkenyl, substituted or unsubstituted C₂₋₆alkynyl, or substituted or unsubstituted C₃₋₁₀carbocyclyl;
   R⁵ is absent, hydrogen, substituted or unsubstituted C₁₋₆alkyl, or halogen;
   R⁶ is hydrogen, substituted or unsubstituted C₁₋₆alkyl, or halogen;
   R^{11a} is hydrogen or —OR^{A11}, wherein R^{A11} is hydrogen or substituted or unsubstituted C₁₋₆alkyl, and R^{11b} is hydrogen; or R^{11a} and R^{11b} are joined to form an oxo (═O) group;
   R¹⁸ is hydrogen, unsubstituted C₂₋₆alkyl, substituted C₁₋₆alkyl, or OR^{A18}, wherein each instance of R^{A18} is independently substituted or unsubstituted C₁₋₆alkyl;
   R¹⁹ is hydrogen, substituted or unsubstituted C₁₋₆alkyl, or OR^{A19}, each instance of
   R^{A19} is independently substituted or unsubstituted C₁₋₆alkyl;
   X is —(C(R^{X})₂)ₙ— or —O—, wherein R^{X} is hydrogen or fluorine, or one R^{X} group and R^{23b} are joined to form a double bond;
   n is 1, 2 or 3;
   each of R^{23a} and R^{23b} is independently hydrogen or fluorine;
   R^{24a} is selected from the group consisting of substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl; and
   R^{24b} is hydrogen or substituted or unsubstituted C₁₋₆alkyl;
   or R^{24a} and R^{24b}, taken together with the carbon atom to which they are attached, form a substituted or unsubstituted C₃₋₆carbocyclyl, or a substituted or unsubstituted 3- to 10-membered heterocyclyl;
   q is 0, 1 or 2;
   r is 1 or 2;
   s is 1 or 2; and
   [symbol] represents a single or double bond, provided if a double bond is present between C5 and C6, R⁵ is absent.

F-99A. A compound of Formula (F-IA):

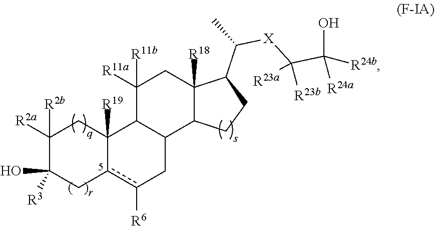

wherein:
   each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein R^{A2} is hydrogen or substituted or unsubstituted C₁₋₆alkyl;
   R³ is substituted or unsubstituted C₁₋₆alkyl, substituted or unsubstituted C₂₋₆ alkenyl, substituted or unsubstituted C₂₋₆alkynyl, or substituted or unsubstituted C₃₋₁₀carbocyclyl;
   R⁶ is hydrogen, substituted or unsubstituted C₁₋₆alkyl, or halogen;
   R^{11a} is hydrogen or —OR^{A11}, wherein R^{A11} is hydrogen or substituted or unsubstituted C₁₋₆alkyl, and R^{11b} is hydrogen; or R^{11a} and R^{11b} are joined to form an oxo (═O) group;
   R¹⁸ is hydrogen, unsubstituted C₂₋₆alkyl, substituted C₁₋₆alkyl, or OR^{A18}, wherein each instance of R^{A18} is independently substituted or unsubstituted C₁₋₆alkyl;
   R¹⁹ is hydrogen, substituted or unsubstituted C₁₋₆alkyl, or OR^{A19}; wherein each instance of R^{A19} is independently substituted or unsubstituted C₁₋₆alkyl;
   X is —(C(R^{X})₂)ₙ— or —O—, wherein R^{X} is hydrogen or fluorine, or one R^{X} group and R^{23b} are joined to form a double bond;
   n is 1, 2 or 3;
   each of R^{23a} and R^{23b} is independently hydrogen or fluorine;
   R^{24a} is selected from the group consisting of substituted or unsubstituted C₁₋₆alkyl, substituted or unsubstituted C₂₋₆alkenyl, substituted or unsubstituted C₂₋₆ alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl; and
   R^{24b} is hydrogen or substituted or unsubstituted C₁₋₆alkyl;
   or R^{24a} and R^{24b}, taken together with the carbon atom to which they are attached, form a substituted or unsubstituted C₃₋₆carbocyclyl, or a substituted or unsubstituted 3- to 10-membered heterocyclyl;
q is 0, 1 or 2;
   r is 1 or 2;
   s is 1 or 2; and
   [symbol] represents a single or double bond, provided if a single bond is present, then the hydrogen at C5 is in the alpha configuration.

F-100. The compound according to embodiment F-99, wherein the compound of Formula (F-I) is a compound of Formula (F-Ia):

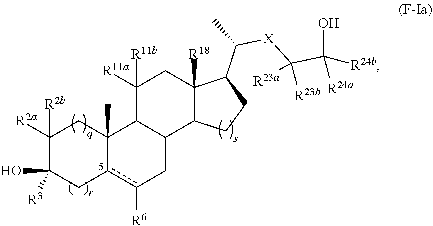

F-101. The compound according to embodiment F-100, wherein the compound of Formula (F-Ia) is a compound of Formula (F-Ia-i):

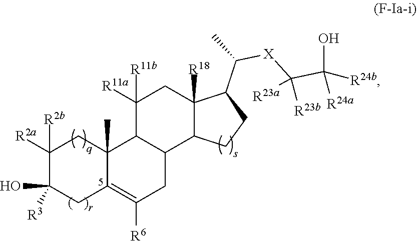

F-102. The compound according to embodiment F-101, wherein the compound of Formula (F-Ia-i) is a compound of Formula (F-Ia-i-1) or Formula (F-Ia-i-2):

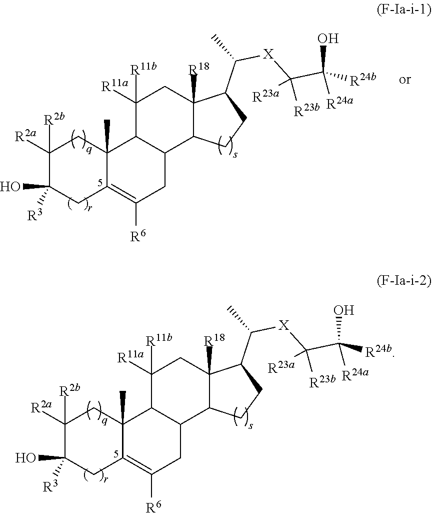

F-103. The compound according to embodiment F-102, wherein the compound of Formula (F-Ia-i) is a compound of Formula (F-Ia-i-1):

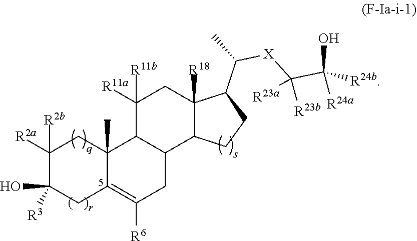

F-104. The compound according to embodiment F-102, wherein the compound of Formula (F-Ia-i) is a compound of Formula (F-Ia-i-2):

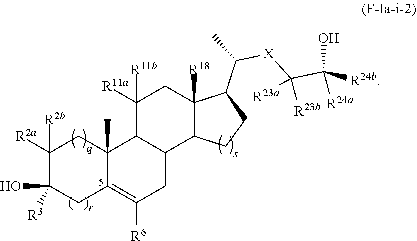

F-105. The compound according to embodiment F-100, wherein the compound of Formula (F-Ia) is a compound of Formula (F-Ia-ii):

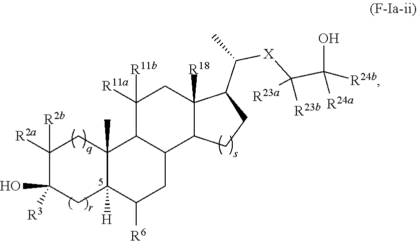

F-106. The compound according to embodiment F-105, wherein the compound of Formula (F-Ia-ii) is a compound of Formula (F-Ia-ii-1) or Formula (F-Ia-ii-2):

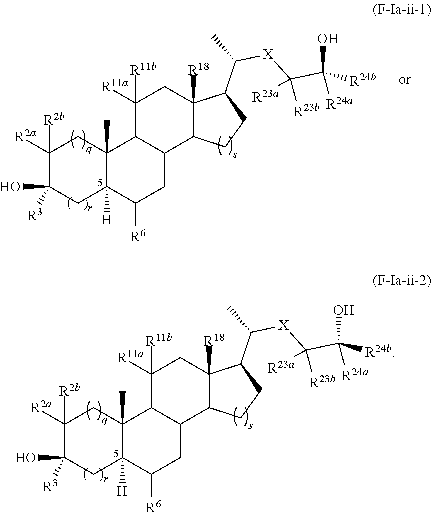

F-107. The compound according to embodiment F-106, wherein the compound of Formula (F-Ia-ii) is a compound of Formula (F-Ia-ii-1):

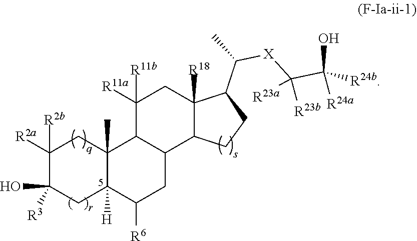

F-108. The compound according to embodiment F-106, wherein the compound of Formula (F-Ia-ii) is a compound of Formula (F-Ia-ii-2):

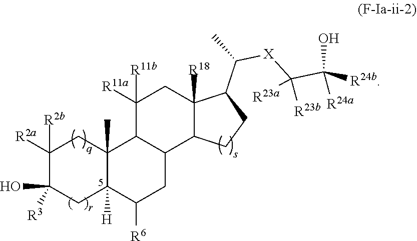

F-109. The compound according to embodiment F-99, wherein the compound of Formula (F-I) is a compound of Formula (F-Ib):

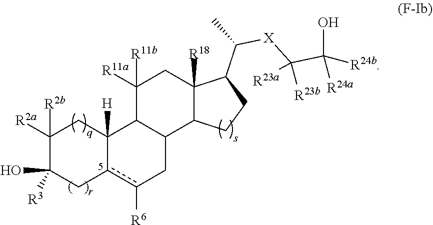

F-110. The compound according to embodiment F-109, wherein the compound of Formula (F-Ib) is a compound of Formula (F-Ib-i):

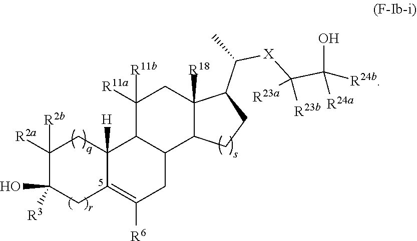

F-111. The compound according to embodiment F-110, wherein the compound of Formula (F-Ib-i) is a compound of Formula (F-Ib-i-1) or Formula (F-Ib-i-2):

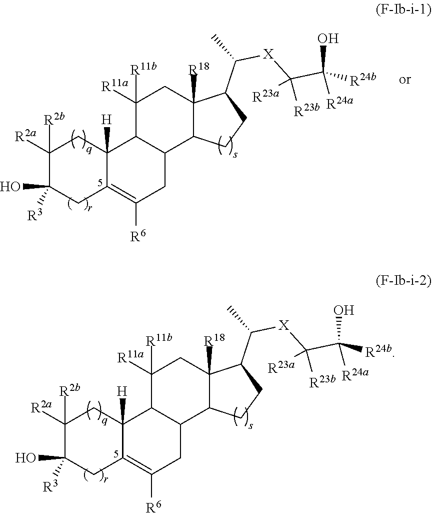

F-112. The compound according to embodiment F-111, wherein the compound of Formula (F-Ib-i) is a compound of Formula (F-Ib-i-1):

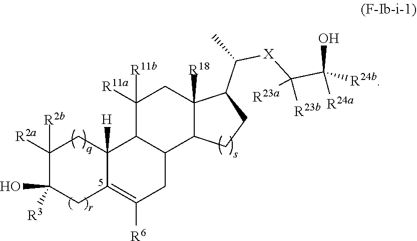

F-113. The compound according to embodiment F-111, wherein the compound of Formula (F-Ib-i) is a compound of Formula (F-Ib-i-2):

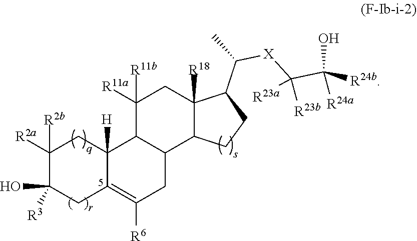

F-114. The compound according to embodiment F-109, wherein the compound of Formula (F-Ib) is a compound of Formula (F-Ib-ii):

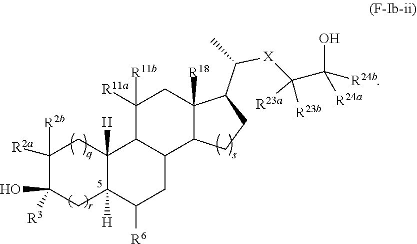

F-115. The compound according to embodiment F-114, wherein the compound of Formula (F-Ib-ii) is a compound of Formula (F-Ib-ii-1) or Formula (F-Ib-ii-2):

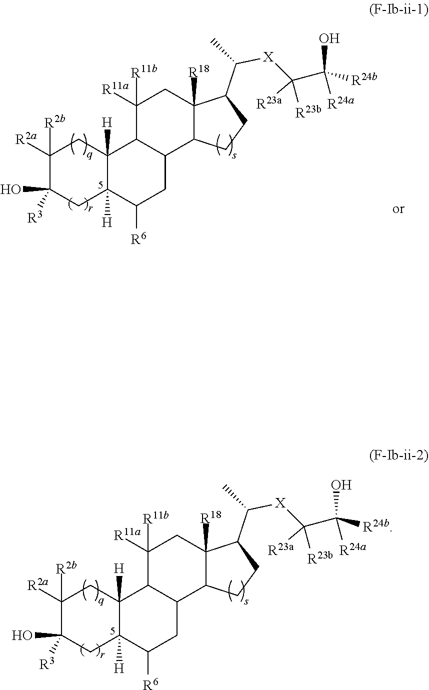

F-116. The compound according to embodiment F-115, wherein the compound of Formula (F-Ib-ii) is a compound of Formula (F-Ib-ii-1):

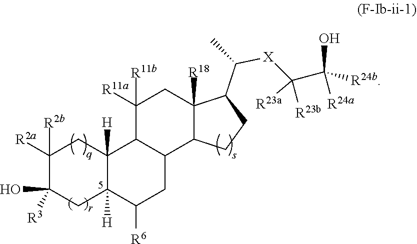

F-117. The compound according to embodiment F-115, wherein the compound of Formula (F-Ib-ii) is a compound of Formula (F-Ib-ii-2):

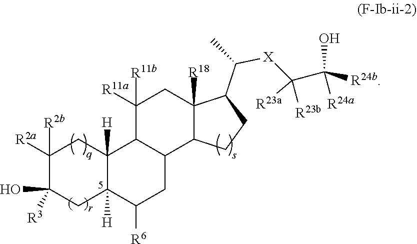

F-118. The compound according to embodiment F-99, wherein the compound of Formula (F-I) is a compound of Formula (F-II):

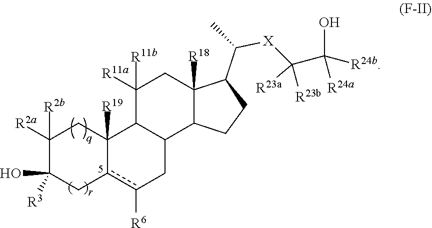

F-119. The compound according to embodiment F-118, wherein the compound of Formula (F-II) is a compound of Formula (F-IIa):

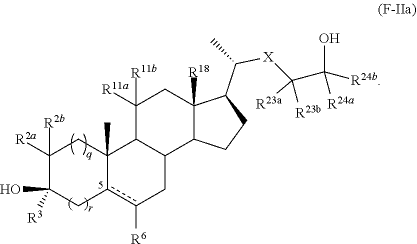

F-120. The compound according to embodiment F-119, wherein the compound of Formula (F-IIa) is a compound of Formula (F-IIa-i):

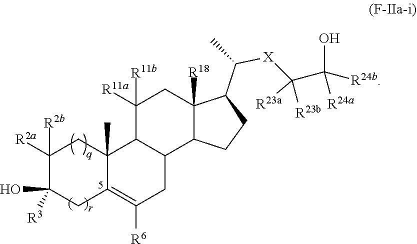

F-121. The compound according to embodiment F-120, wherein the compound of Formula (F-IIa-i) is a compound of Formula (F-IIa-i-1) or Formula (F-IIa-i-2):

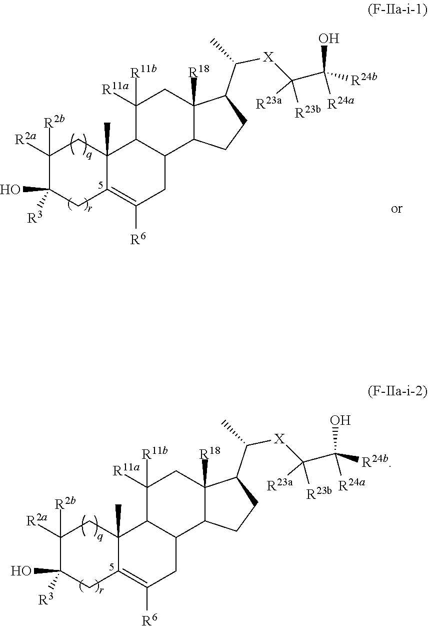

F-122. The compound according to embodiment F-121, wherein the compound of Formula (F-IIa-i) is a compound of Formula (F-Ia-i-1):

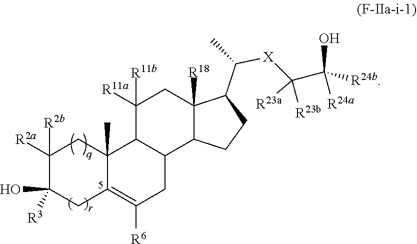

F-123. The compound according to embodiment F-121, wherein the compound of Formula (F-IIa-i) is a compound of Formula (F-IIa-i-2):

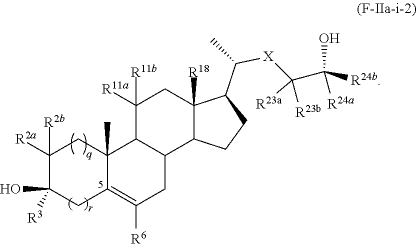

F-124. The compound according to embodiment F-123, wherein the compound of Formula (F-IIa) is a compound of Formula (F-IIa-ii):

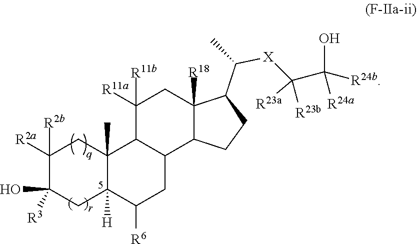

F-125. The compound according to embodiment F-124, wherein the compound of Formula (F-IIa-ii) is a compound of Formula (F-IIa-ii-1) or Formula (F-IIa-ii-2):

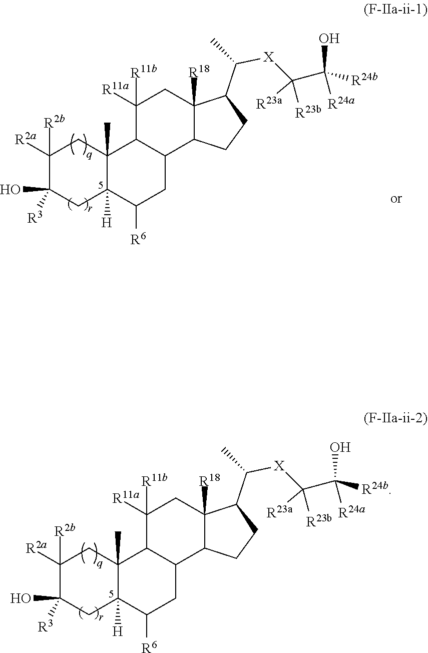

F-126. The compound according to embodiment F-125, wherein the compound of Formula (F-IIa-ii) is a compound of Formula (F-IIa-ii-1):

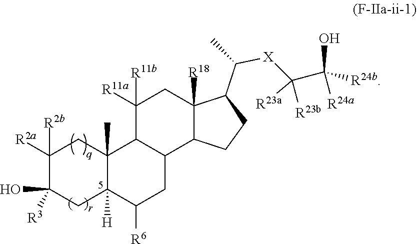

F-127. The compound according to embodiment F-125, wherein the compound of Formula (F-IIa-ii) is a compound of Formula (F-IIa-ii-2):

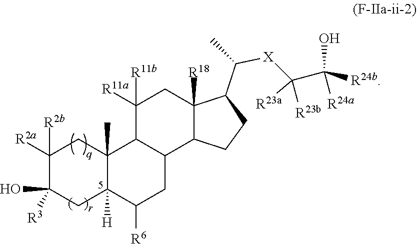

F-128. The compound according to embodiment F-118, wherein the compound of Formula (F-II) is a compound of Formula (F-IIb):

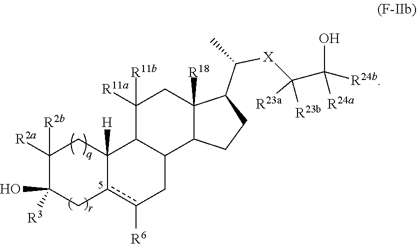

F-129. The compound according to embodiment F-128, wherein the compound of Formula (F-IIb) is a compound of Formula (F-IIb-i):

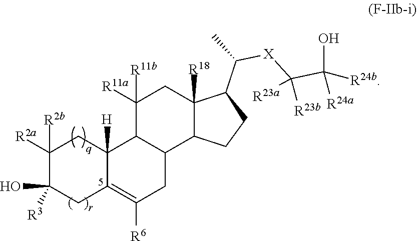

F-130. The compound according to embodiment F-129, wherein the compound of Formula (F-IIb-i) is a compound of Formula (F-IIb-i-1) or Formula (F-IIb-i-2):

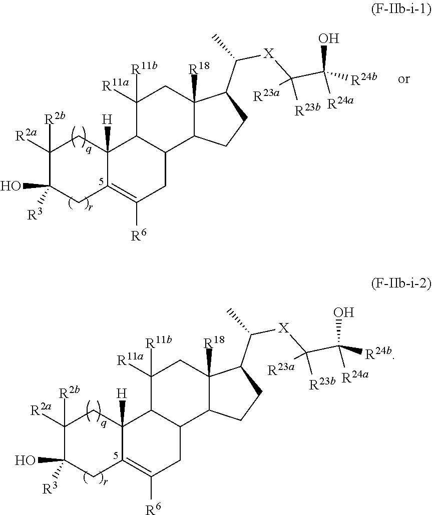

F-131. The compound according to embodiment F-130, wherein the compound of Formula (F-IIb-i) is a compound of Formula (F-IIb-i-1):

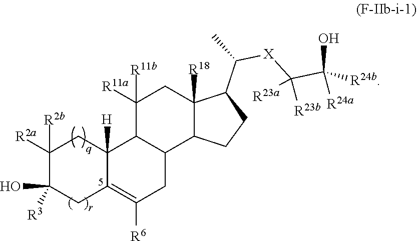

F-132. The compound according to embodiment F-131, wherein the compound of Formula (F-IIb-i) is a compound of Formula (F-IIb-i-2):

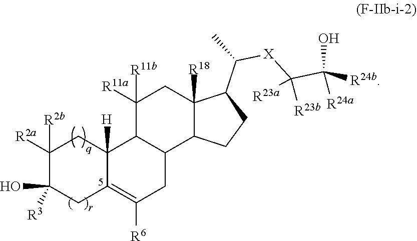

F-133. The compound according to embodiment F-128, wherein the compound of Formula (F-IIb) is a compound of Formula (F-IIb-ii):

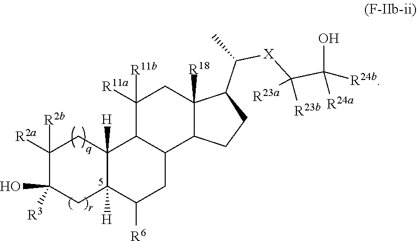

F-134. The compound according to embodiment F-133, wherein the compound of Formula (F-IIb-ii) is a compound of Formula (F-IIb-ii-1) or Formula (F-IIb-ii-2):

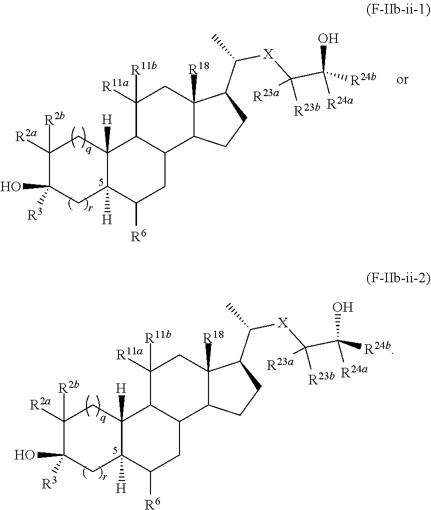

F-135. The compound according to embodiment F-134, wherein the compound of Formula (F-IIb-ii) is a compound of Formula (F-IIb-ii-1):

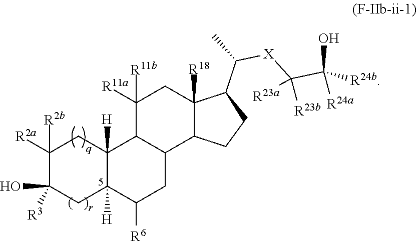

F-136. The compound according to embodiment F-135, wherein the compound of Formula (F-IIb-ii) is a compound of Formula (F-IIb-ii-2):

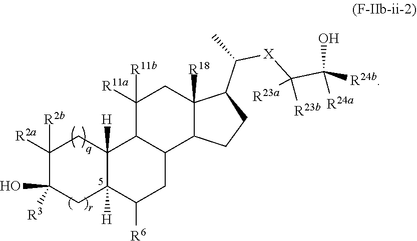

F-137. The compound according to embodiment F-99, wherein the compound of Formula (F-I) is a compound of Formula (F-III):

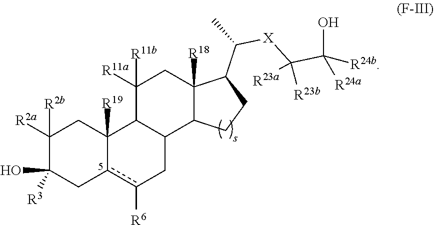

F-138. The compound according to embodiment F-137, wherein the compound of Formula (F-III) is a compound of Formula (F-IIIa):

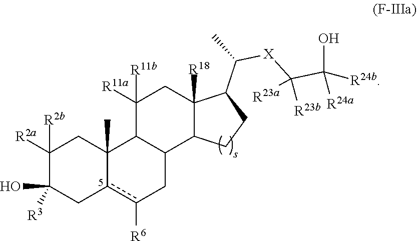

F-139. The compound according to embodiment F-138, wherein the compound of Formula (F-IIIa) is a compound of Formula (F-IIIa-i):

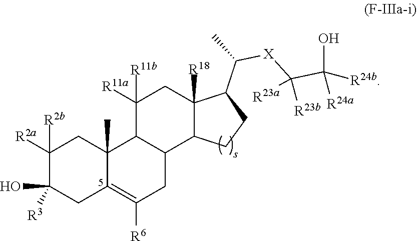

F-140. The compound according to embodiment F-139, wherein the compound of Formula (F-IIIa-i) is a compound of Formula (F-IIIa-i-1) or Formula (F-IIIa-i-2):

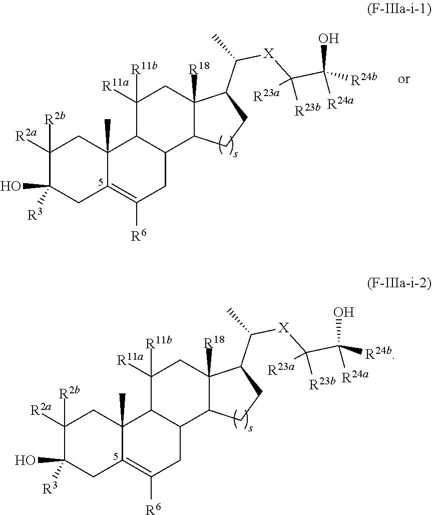

F-141. The compound according to embodiment F-140, wherein the compound of Formula (F-IIIa-i) is a compound of Formula (F-IIIa-i-1):

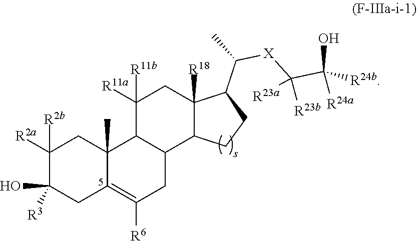

F-142. The compound according to embodiment F-140, wherein the compound of Formula (F-IIIa-i) is a compound of Formula (F-IIIa-i-2):

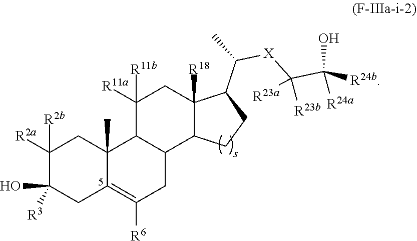

F-143. The compound according to embodiment F-138, wherein the compound of Formula (F-IIIa) is a compound of Formula (F-IIIa-ii):

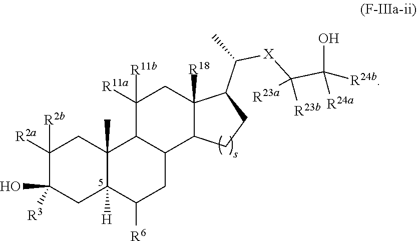

F-144. The compound according to embodiment F-143, wherein the compound of Formula (F-IIIa-ii) is a compound of Formula (F-IIIa-ii-1) or Formula (F-IIIa-ii-2):

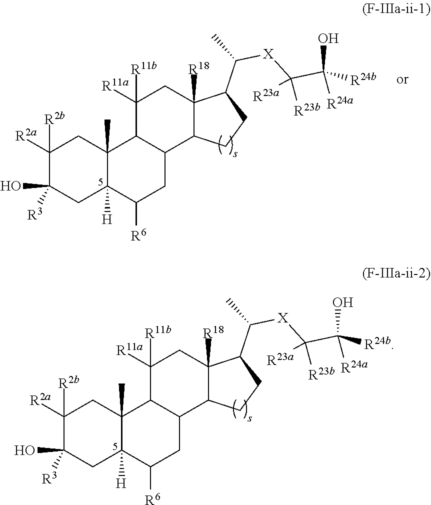

F-145. The compound according to embodiment F-144, wherein the compound of Formula (F-IIIa-ii) is a compound of Formula (F-IIIa-ii-1):

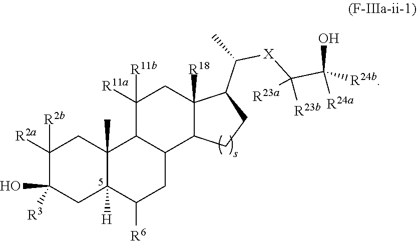

F-146. The compound according to embodiment F-144, wherein the compound of Formula (F-IIIa-ii) is a compound of Formula (F-IIIa-ii-2):

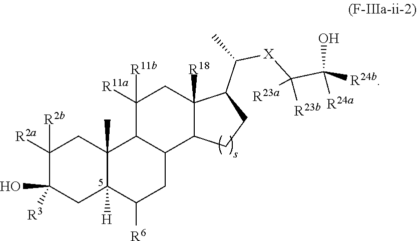

F-147. The compound according to embodiment F-137, wherein the compound of Formula (F-III) is a compound of Formula (F-IIIb):

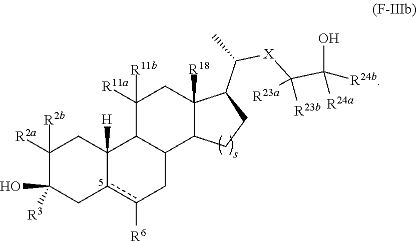

F-148. The compound according to embodiment F-147, wherein the compound of Formula (F-IIIb) is a compound of Formula (F-IIIb-i):

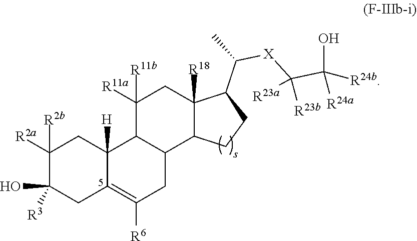

F-149. The compound according to embodiment F-148, wherein the compound of Formula (F-IIIb-i) is a compound of Formula (F-IIIb-i-1) or Formula (F-IIIb-i-2):

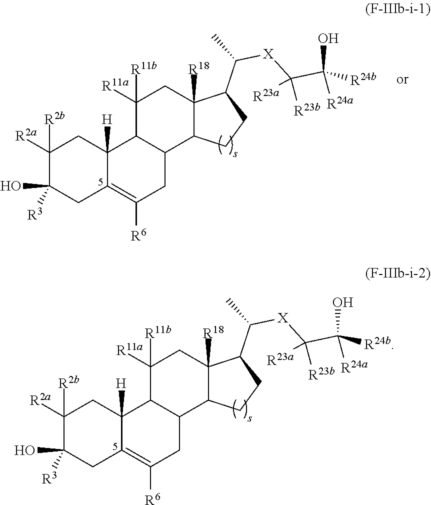

F-150. The compound according to embodiment F-149, wherein the compound of Formula (F-IIIb-i) is a compound of Formula (F-IIIb-i-1):

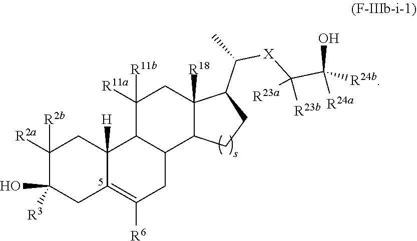

F-151. The compound according to embodiment F-149, wherein the compound of Formula (F-IIIb-i) is a compound of Formula (F-IIIb-i-2):

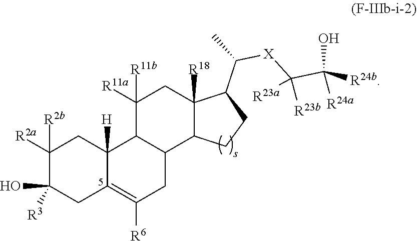

F-152. The compound according to embodiment F-147, wherein the compound of Formula (F-IIIb) is a compound of Formula (F-IIIb-ii):

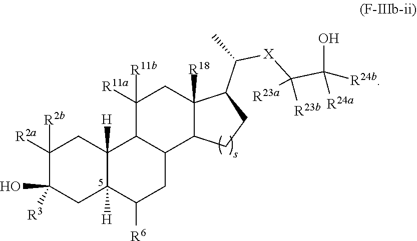

F-153. The compound according to embodiment F-152, wherein the compound of Formula (F-IIIb-ii) is a compound of Formula (F-IIIb-ii-1) or Formula (F-IIIb-ii-2):

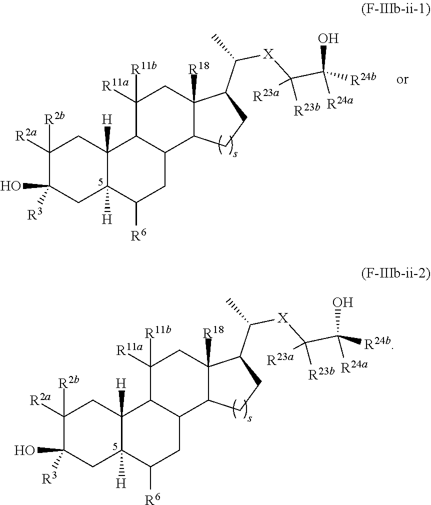

F-154. The compound according to embodiment F-153, wherein the compound of Formula (F-IIIb-ii) is a compound of Formula (F-IIIb-ii-1):

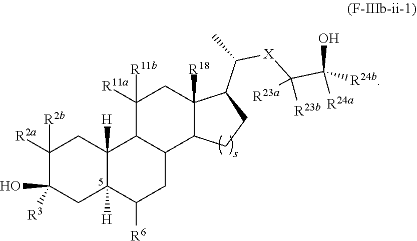

F-155. The compound according to embodiment F-153, wherein the compound of Formula (F-IIIb-ii) is a compound of Formula (F-IIIb-ii-2):

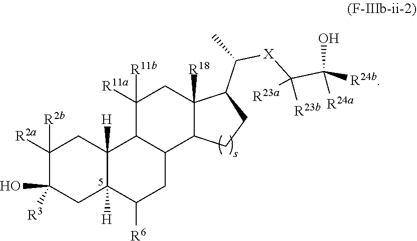

F-156. The compound according to embodiment F-99, wherein the compound of Formula (F-I) is a compound of Formula (IV):

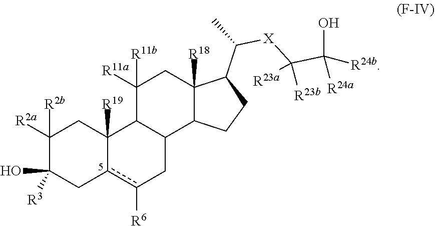

F-157. The compound according to embodiment F-156, wherein the compound of Formula (F-IV) is a compound of Formula (F-IVa):

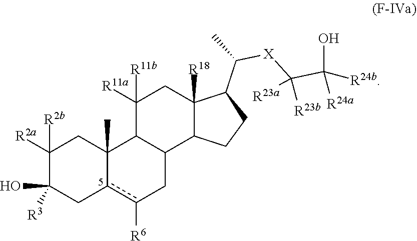

F-158. The compound according to embodiment F-157, wherein the compound of Formula (F-IVa) is a compound of Formula (F-IVa-i):

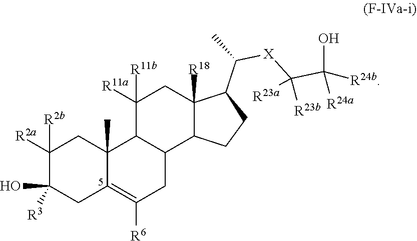

F-159. The compound according to embodiment F-158, wherein the compound of Formula (F-IVa-i) is a compound of Formula (F-IVa-i-1) or Formula (F-IVa-i-2):

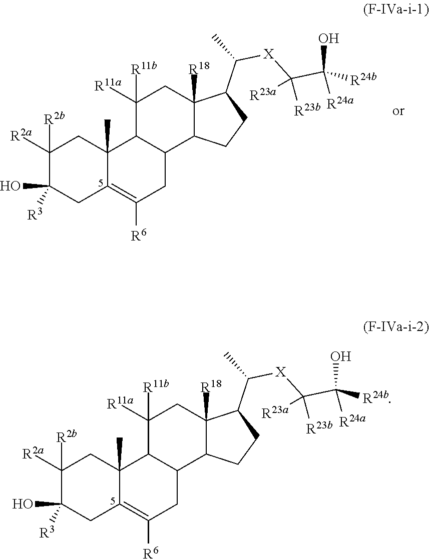

F-160. The compound according to embodiment F-159, wherein the compound of Formula (F-IVa-i) is a compound of Formula (F-IVa-i-1):

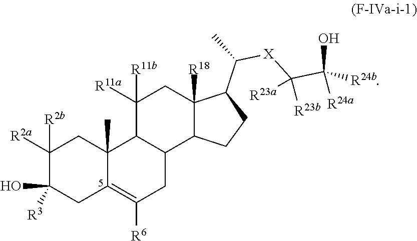

F-161. The compound according to embodiment F-159, wherein the compound of Formula (F-IVa-i) is a compound of Formula (F-IVa-i-2):

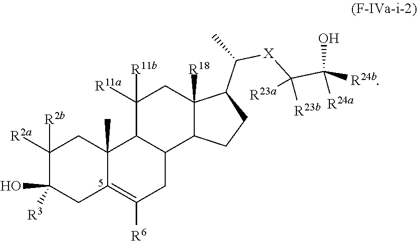

F-162. The compound according to embodiment F-157, wherein the compound of Formula (F-IVa) is a compound of Formula (F-IVa-ii):

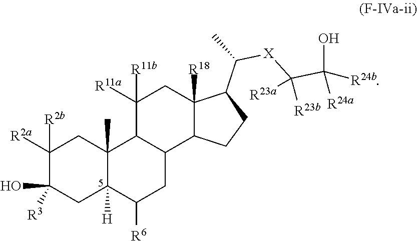

F-163. The compound according to embodiment F-162, wherein the compound of Formula (F-IVa-ii) is a compound of Formula (F-IVa-ii-1) or Formula (F-IVa-ii-2):

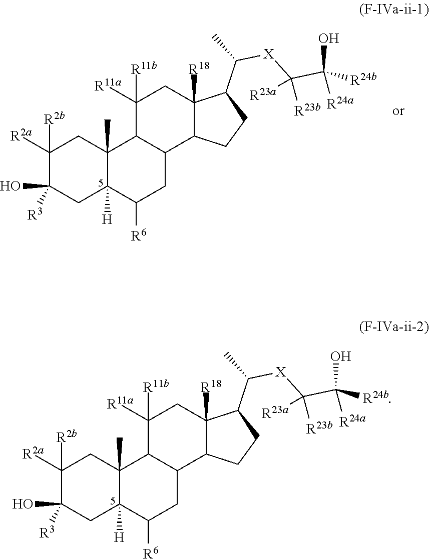

F-164. The compound according to embodiment F-163, wherein the compound of Formula (F-IVa-ii) is a compound of Formula (F-IVa-ii-1):

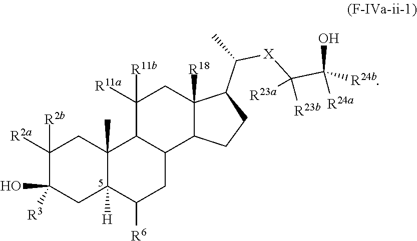

F-165. The compound according to embodiment F-163, wherein the compound of Formula (F-IVa-ii) is a compound of Formula (F-IVa-ii-2):

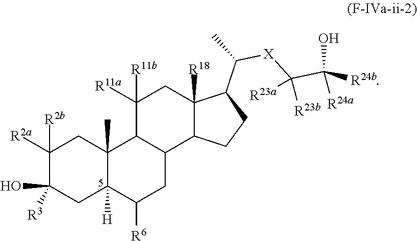

F-166. The compound according to embodiment F-156, wherein the compound of Formula (F-IV) is a compound of Formula (F-IVb):

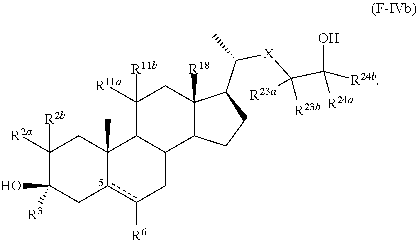

F-167. The compound according to embodiment F-166, wherein the compound of Formula (F-IVb) is a compound of Formula (F-IVb-i):

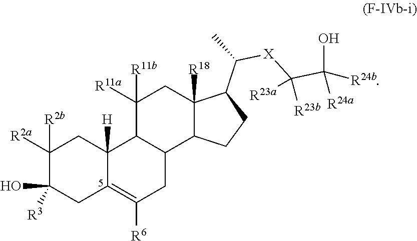

F-168. The compound according to embodiment F-167, wherein the compound of Formula (F-IVb-i) is a compound of Formula (F-IVb-i-1) or Formula (F-IVb-i-2):

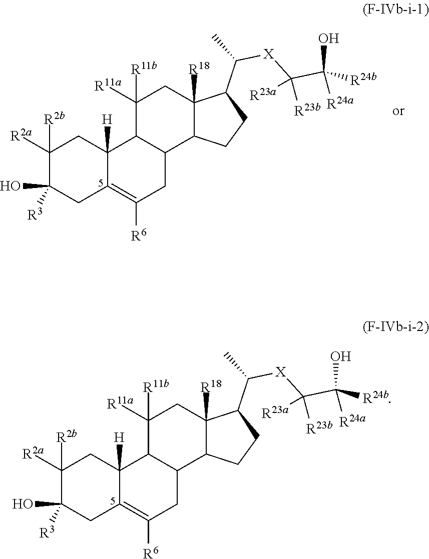

F-169. The compound according to embodiment F-167, wherein the compound of Formula (F-IVb-i) is a compound of Formula (F-IVb-i-1):

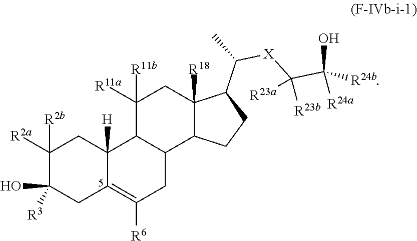

F-170. The compound according to embodiment F-167, wherein the compound of Formula (F-IVb-i) is a compound of Formula (F-IVb-i-2):

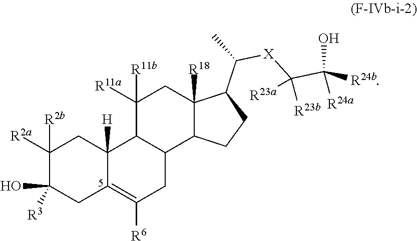

F-171. The compound according to embodiment F-166, wherein the compound of Formula (F-IVb) is a compound of Formula (F-IVb-ii):

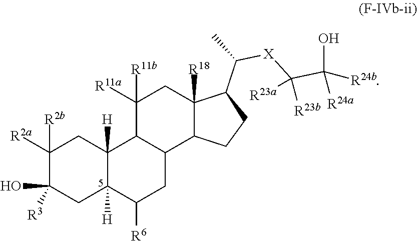

F-172. The compound according to embodiment F-171, wherein the compound of Formula (F-IVb-ii) is a compound of Formula (F-IVb-ii-1) or Formula (F-IVb-ii-2):

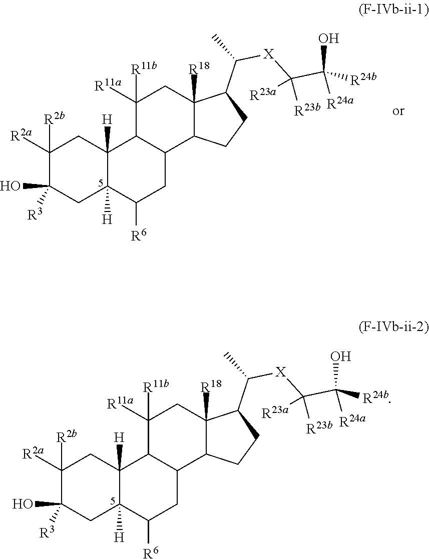

F-173. The compound according to embodiment F-172, wherein the compound of Formula (F-IVb-ii) is a compound of Formula (F-IVb-ii-1):

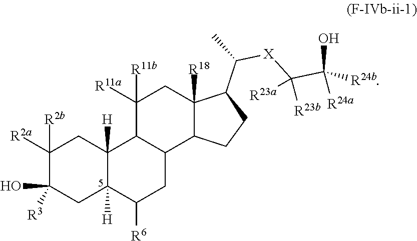

F-174. The compound according to embodiment F-172, wherein the compound of Formula (F-IVb-ii) is a compound of Formula (F-IVb-ii-2):

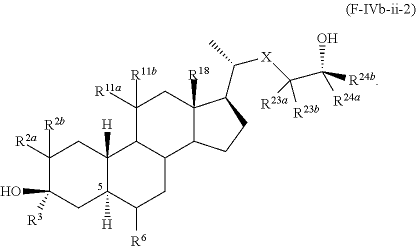

F-175. The compound according to embodiment F-99, wherein the compound of Formula (F-I) is a compound of Formula (F-V):

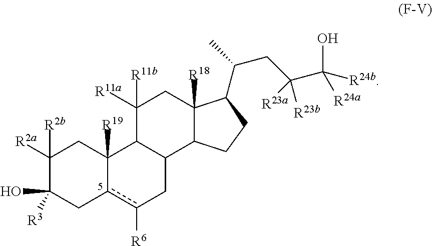

F-176. The compound according to embodiment F-175, wherein the compound of Formula (F-V) is a compound of Formula (F-Va):

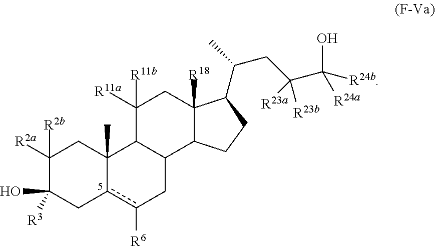

F-177. The compound according to embodiment F-176, wherein the compound of Formula (F-Va) is a compound of Formula (F-Va-i):

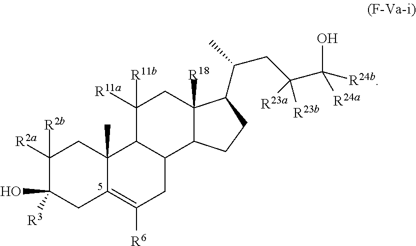

F-178. The compound according to embodiment F-177, wherein the compound of Formula (F-Va-i) is a compound of Formula (F-Va-i-1) or Formula (F-Va-i-2):

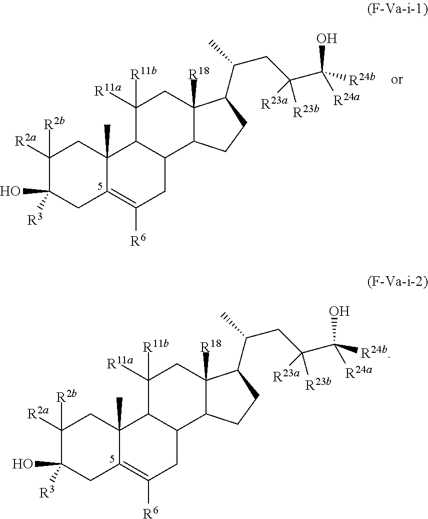

F-179. The compound according to embodiment F-178, wherein the compound of Formula (F-Va-i) is a compound of Formula (F-Va-i-1):

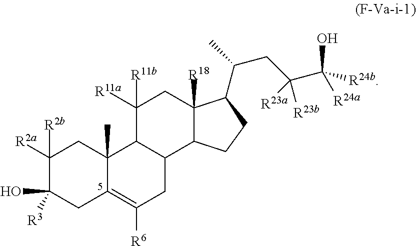

F-180. The compound according to embodiment F-178, wherein the compound of Formula (F-Va-i) is a compound of Formula (F-Va-i-2):

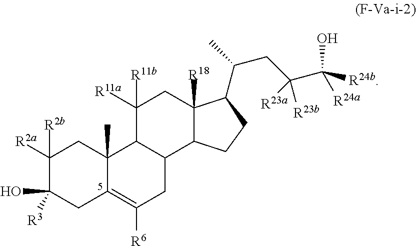

F-181. The compound according to embodiment F-176, wherein the compound of Formula (F-Va) is a compound of Formula (F-Va-ii):

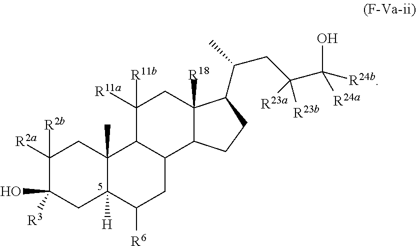

F-182. The compound according to embodiment F-181, wherein the compound of Formula (F-Va-ii) is a compound of Formula (F-Va-ii-1) or Formula (F-Va-ii-2):

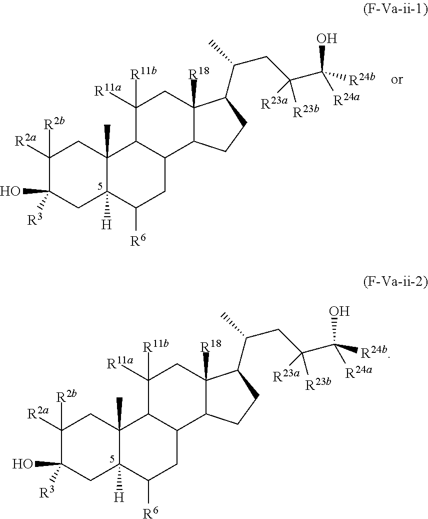

F-183. The compound according to embodiment F-182, wherein the compound of Formula (F-Va-ii) is a compound of Formula (F-Va-ii-1):

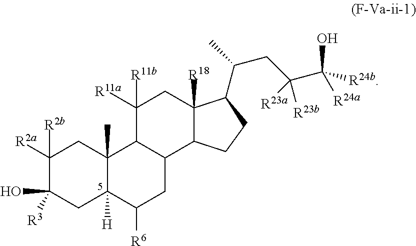

F-184. The compound according to embodiment F-182, wherein the compound of Formula (F-Va-ii) is a compound of Formula (F-Va-ii-2):

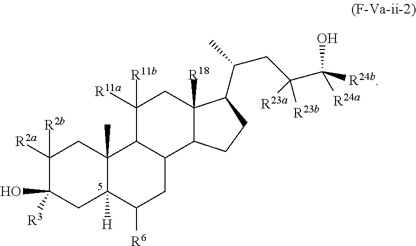

F-185. The compound according to embodiment F-175, wherein the compound of Formula (F-V) is a compound of Formula (F-Vb):

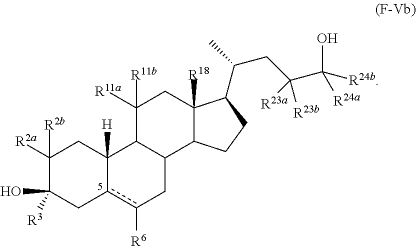

F-186. The compound according to embodiment F-185, wherein the compound of Formula (F-Vb) is a compound of Formula (F-Vb-i):

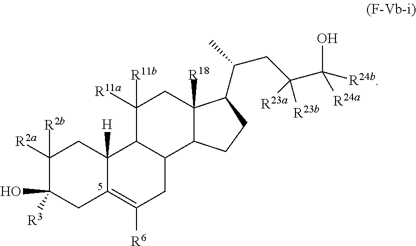

F-187. The compound according to embodiment F-186, wherein the compound of Formula (F-Vb-i) is a compound of Formula (F-Vb-i-1) or Formula (F-Vb-i-2):

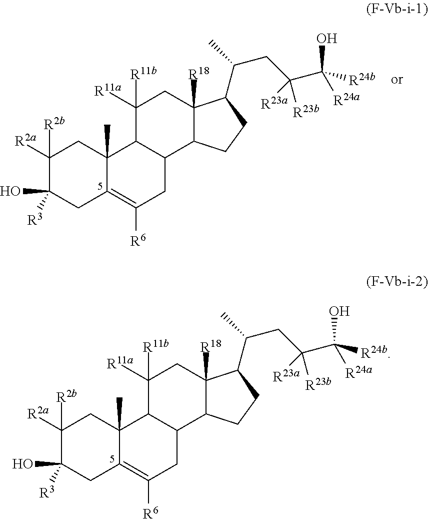

F-188. The compound according to embodiment F-187, wherein the compound of Formula (F-Vb-i) is a compound of Formula (F-Vb-i-1):

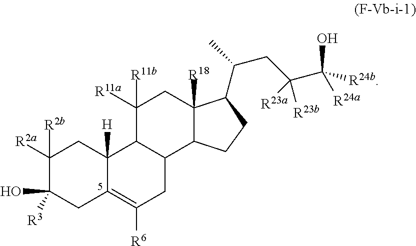

F-189. The compound according to embodiment F-187, wherein the compound of Formula (F-Vb-i) is a compound of Formula (F-Vb-i-2):

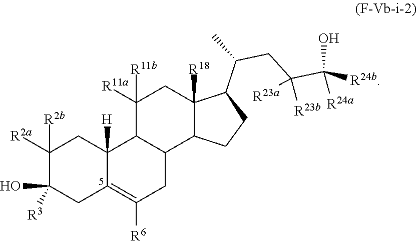

F-190. The compound according to embodiment F-185, wherein the compound of Formula (F-Vb) is a compound of Formula (F-Vb-ii):

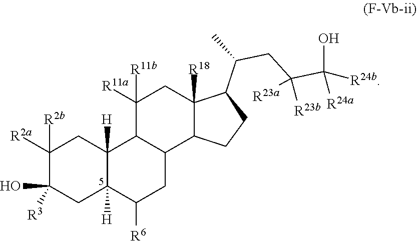

F-191. The compound according to embodiment F-190, wherein the compound of Formula (F-Vb-ii) is a compound of Formula (F-Vb-ii-1) or Formula (F-Vb-ii-2):

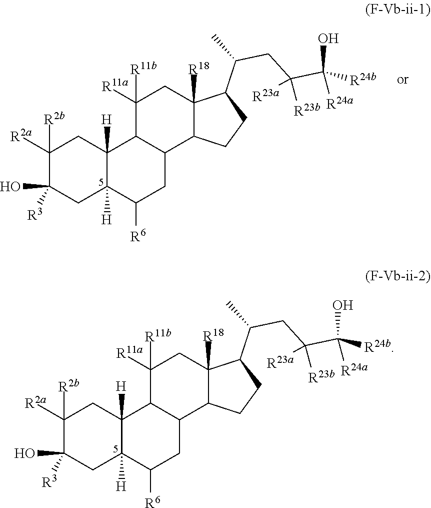

F-192. The compound according to embodiment F-191, wherein the compound of Formula (F-Vb-ii) is a compound of Formula (F-Vb-ii-1):

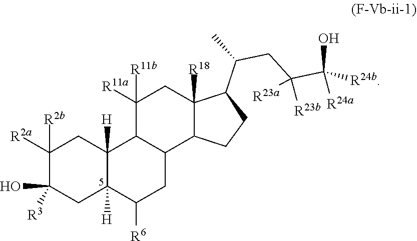

F-193. The compound according to embodiment F-191, wherein the compound of Formula (F-Vb-ii) is a compound of Formula (F-Vb-ii-2):

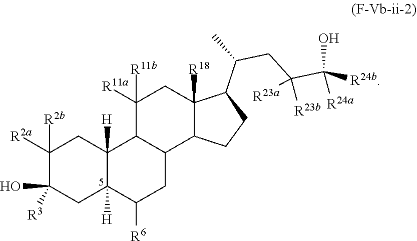

F-194. The compound according to any one of embodiments F-99 to F-193, wherein each of R^{2a} and R^{2b} is hydrogen.

F-195. The compound according to any one of embodiments F-99 to F-193, wherein R³ is unsubstituted C₁₋₆alkyl.

F-196. The compound according to embodiment F-195, wherein R³ is —CH₃, —CH₂CH₃, or —CH₂CH₂CH₃.

F-197. The compound according to embodiment F-196, wherein R³ is —CH₃.

F-198. The compound according to embodiment F-196, wherein R³ is —CH₂CH₃.

F-199. The compound according to any one of embodiments F-99 to F-193, wherein each of R^{11a} and R^{11b} is hydrogen.

F-200. The compound according to embodiments F-99 to F-193, wherein R⁶ is hydrogen.

F-201. The compound according to any one of embodiments F-99 to F-193, wherein R¹⁸ is hydrogen, unsubstituted C₂₋₆alkyl, C₁₋₆alkyl substituted with OR^{A18}, or —OR^{A18}, wherein each instance of R^{A18} is independently unsubstituted C₁₋₆alkyl.

F-202. The compound according to embodiment F-201, wherein R¹⁸ is hydrogen, —CH₂OCH₃, —OCH₃, —CH₂CH₃, or —CH(CH₃)₂.

F-203. The compound according to embodiment F-202, wherein R¹⁸ is hydrogen.

F-204. The compound according to embodiment F-202, wherein R¹⁸ is —CH₂CH₃.

F-205. The compound according to any one of embodiments F-99 to F-193, wherein each of R^{23a} and R^{23b} is hydrogen.

F-206. The compound according to any one of embodiments F-99 to F-193, wherein at least one of R^{23a} and R^{23b} is fluorine.

F-207. The compound according to any one of embodiments F-99 to F-193, wherein each of R^{23a} and R^{23b} is fluorine.

F-208. The compound according to any one of embodiments F-99 to F-193, wherein R^{24a} is a non-hydrogen group substituted with fluorine.

F-209. The compound according to any one of embodiments F-99 to F-193, wherein R^{24a} is a non-hydrogen group substituted with one or more —OR^{A24} groups, wherein R^{A24} is hydrogen or substituted or unsubstituted C₁₋₆alkyl.

F-210. The compound according to any one of embodiments F-99 to F-193, wherein R^{24a} is —CH₂OR^{A24}, —CH₂CH₂OR^{A24}, or —CH₂CH₂CH₂OR^{A24}.

F-211. The compound according to any one of embodiments F-99 to F-193, wherein R^{24a} is a substituted or unsubstituted C14alkyl, substituted or unsubstituted C₂₋₃ alkenyl, substituted or unsubstituted C₂₋₃ alkynyl, or substituted or unsubstituted C₃carbocyclyl.

F-212. The compound according to embodiment F-211, wherein R^{24a} is —CF₃.

F-213. The compound according to embodiment F-211, wherein R^{24a} is —CH(CH₃)₂.

F-214. The compound according to any one of embodiments F-99 to F-193, wherein R^{24b} is hydrogen.

F-215. The compound according to any one of embodiments F-99 to F-193, wherein R^{24b} is —CH₃ or —CF₃.

F-216. The compound according to any one of embodiments F-99 to F-193, wherein R^{24b} is —CH₃.

F-217. The compound according to any one of embodiments F-99 to F-193, wherein R^{24a} is —CH(CH₃)₂ or —CF₃, and R^{24b} is hydrogen.

F-218. The compound according to any one of embodiments F-99 to F-193, wherein R^{24a} is a non-hydrogen group substituted with fluorine, and R^{24b} is —CH₃.

F-219. The compound according to any one of embodiments F-99 to F-193, wherein R^{24a} is substituted with one or more —OR^{A24} groups, wherein R^{A24} is hydrogen or substituted or unsubstituted C₁₋₆alkyl.

F-220. The compound according to any one of embodiments F-99 to F-193, wherein R^{24a} is a substituted or unsubstituted C₁₋₆alkyl, substituted or unsubstituted C₂₋₃ alkenyl, substituted or unsubstituted C₂₋₃ alkynyl, or substituted or unsubstituted C₃carbocyclyl, and R^{24b} is —H.

F-221. The compound according to any one of embodiments F-99 to F-193, wherein when R^{24b} is —H, R^{24a} is not C₁₋₆alkyl substituted with CN.

F-222. The compound according to any one of embodiments F-99 to F-193, wherein R³ is C₁₋₃ alkyl, R^{24a} is a non-hydrogen group substituted with fluorine, and R^{24b} is —CH₃.

F-223. The compound according to any one of embodiments F-99 to F-193, wherein R³ is C₁₋₃ alkyl, R^{24a} is a non-hydrogen group substituted with fluorine, and R^{24b} is hydrogen.

F-224. The compound according to any one of embodiments F-99 to F-193, wherein R³ is —CH₃ or —CH₂CH₃, R^{24a} is —CH(CH₃)₂ or —CF₃, and R^{24b} is hydrogen.

F-225. The compound according to embodiment F-99, wherein the compound of formula (F-I) is selected from the group consisting of Compounds F-1-F-14 of Table F-1.

F-226. The compound according to embodiment F-99, wherein the compound of formula (F-I) is selected from the group consisting of Compounds F1-F12 of Table F-1.

F-227. A pharmaceutical composition comprising a compound according to any one of embodiments F-99 to F-226, and a pharmaceutically acceptable carrier.

F-228. A method for treating a CNS-related condition in a subject, comprising administering to the subject an effective amount of a compound according to any one of embodiments F-99 to F-226, or a pharmaceutical composition according to embodiment F-227.

F-229. The method according to embodiment F-228, wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease), and tinnitus.

F-230. A method of inducing sedation or anesthesia in a subject, comprising administering to the subject an effective amount of a compound according to any one of embodiments F-99 to F-226, or a pharmaceutical composition according to embodiment F-227.

F-231. A compound according to any one of embodiments F-99 to F-226, or a pharmaceutical composition according to embodiment F-227, for use in treating a CNS-related condition in a subject.

F-232. The compound or pharmaceutical composition for use according to embodiment F-231, wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease), and tinnitus.

F-233. A compound according to any one of embodiments F-99 to F-226, or a pharmaceutical composition according to embodiment F-227, for use in inducing sedation or anesthesia in a subject.

F-234. Use of a compound according to any one of embodiments F-99 to F-226, or a pharmaceutical composition according to embodiment F-227, for the manufacture of a medicament for treating a CNS-related condition in a subject.

F-235. The use according to embodiment F-234, wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease), and tinnitus.

F-236. Use of a compound according to any one of embodiments F-99 to F-226, or a pharmaceutical composition according to embodiment F-227, for the manufacture of a medicament for inducing sedation or anesthesia in a subject.

G-1. A compound of Formula (G-I):

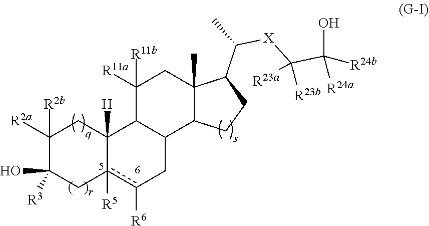

or a pharmaceutically acceptable salt thereof;
wherein:
   each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein R^{A2} is hydrogen or substituted or unsubstituted C₁₋₆alkyl;
   R³ is substituted or unsubstituted C₁₋₆alkyl, substituted or unsubstituted C₂₋₆alkenyl, or substituted or unsubstituted C₂₋₆alkynyl;
   R⁵ is absent, hydrogen, substituted or unsubstituted C₁₋₆alkyl, or halogen; R⁶ is hydrogen, substituted or unsubstituted C₁₋₆alkyl, or halogen;
   R¹⁹ is hydrogen, substituted or unsubstituted C₂₋₆alkyl, or OR^{A19}; wherein each instance of R^{A19} is independently substituted or unsubstituted C₁₋₆alkyl;
   R^{11a} is hydrogen or —OR^{A11}, wherein R^{A11} is hydrogen or substituted or unsubstituted C₁₋₆alkyl, and R^{11b} is hydrogen; or R^{11a} and R^{11b} are joined to form an oxo (═O) group;
   X is —(C(R^{X})₂)ₙ— or —O—, wherein R^{X} is hydrogen or fluorine, or one R^{X} group and R^{23b} are joined to form a double bond;
n is 1, 2 or 3;
   each of R^{23a} and R^{23b} is independently hydrogen or fluorine;
   R^{24a} is selected from the group consisting of substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl; and
   R^{24b} is hydrogen or substituted or unsubstituted C₁₋₆alkyl;
   or R^{24a} and R^{24b}, taken together with the carbon atom to which they are attached, form a substituted or unsubstituted C₃₋₆carbocyclyl;
   q is 0, 1 or 2;
   r is 1 or 2;
   s is 1, 2, 3 or 4; and
   [symbol] represents a single or double bond; provided if the bond between C5 and

C6 is a double bond, R⁵ is absent.

G-2. The compound or pharmaceutically acceptable salt thereof according to embodiment G-1, wherein the compound of Formula (G-I) is a compound of Formula (G-Ia):

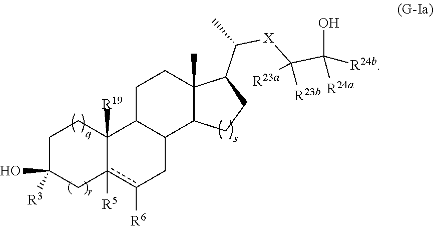

G-3. The compound or pharmaceutically acceptable salt thereof according to embodiment G-2, wherein the compound of Formula (G-Ia) is a compound of Formula (G-Ia-1):

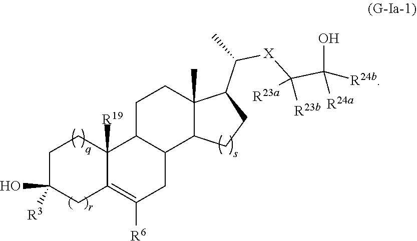

G-4. The compound or pharmaceutically acceptable salt thereof according to embodiment G-2, wherein the compound of Formula (G-Ia) is a compound of Formula (G-Ia-2):

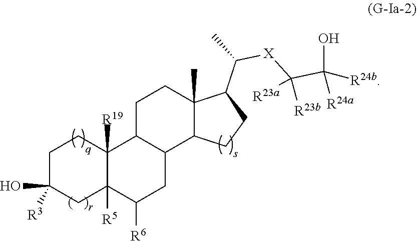

G-5. The compound or pharmaceutically acceptable salt thereof according to embodiment G-1, wherein the compound of Formula (G-I) is a compound of Formula (G-II):

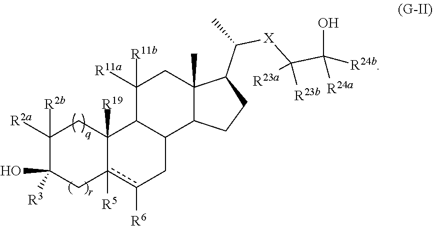

G-6. The compound or pharmaceutically acceptable salt thereof according to embodiment G-5, wherein the compound of Formula (G-II) is a compound of Formula (G-IIa):

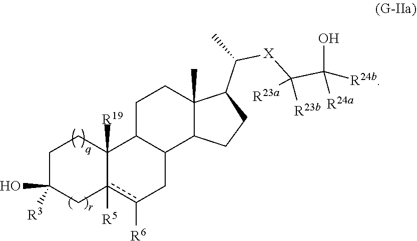

G-7. The compound or pharmaceutically acceptable salt thereof according to embodiment G-6, wherein the compound of Formula (G-IIa) is a compound of Formula (G-Ia-1):

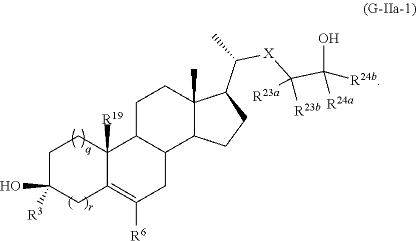

G-8. The compound or pharmaceutically acceptable salt thereof according to embodiment G-6, wherein the compound of Formula (G-IIa) is a compound of Formula (G-IIa-2):

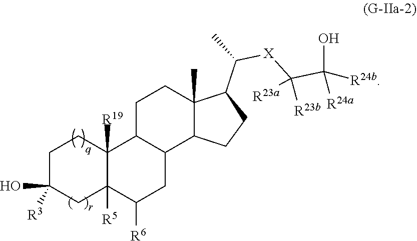

G-9. The compound or pharmaceutically acceptable salt thereof according to embodiment G-1, wherein the compound of Formula (G-I) is a compound of Formula (G-III):

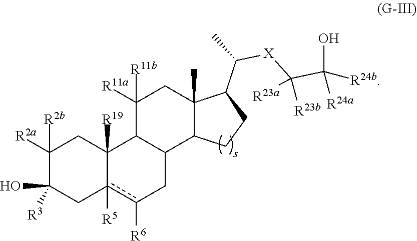

G-10. The compound or pharmaceutically acceptable salt thereof according to embodiment G-9, wherein the compound of Formula (G-III) is a compound of Formula (G-IIIa):

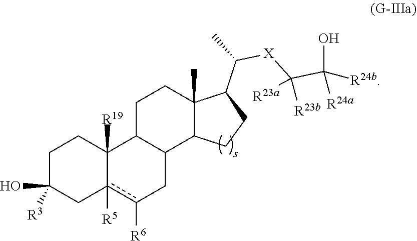

G-11. The compound or pharmaceutically acceptable salt thereof according to embodiment G-10, wherein the compound of Formula (G-IIIa) is a compound of Formula (G-IIIa-1):

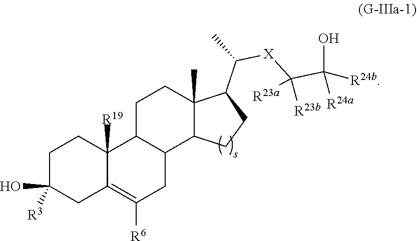

G-12. The compound or pharmaceutically acceptable salt thereof according to embodiment G-10, wherein the compound of Formula (G-IIIa) is a compound of Formula (G-IIIa-2):

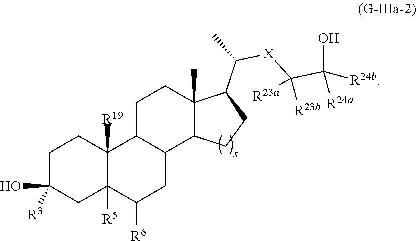

G-13. The compound or pharmaceutically acceptable salt thereof according to embodiment G-1, wherein the compound of Formula (G-I) is a compound of Formula (G-IV):

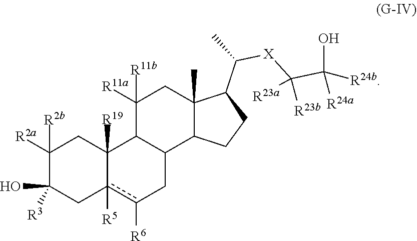

G-14. The compound or pharmaceutically acceptable salt thereof according to embodiment G-13, wherein the compound of Formula (G-IV) is a compound of Formula (G-IVa):

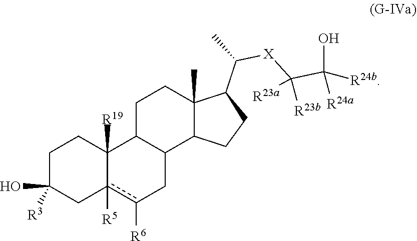

G-15. The compound or pharmaceutically acceptable salt thereof according to embodiment G-14, wherein the compound of Formula (G-IVa) is a compound of Formula (G-IVa-1):

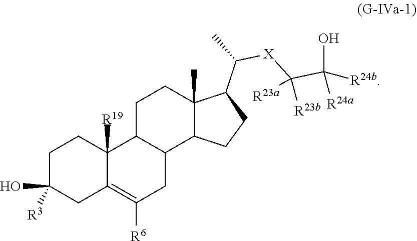

G-16. The compound or pharmaceutically acceptable salt thereof according to embodiment G-14, wherein the compound of Formula (G-IVa) is a compound of Formula (G-IVa-2):

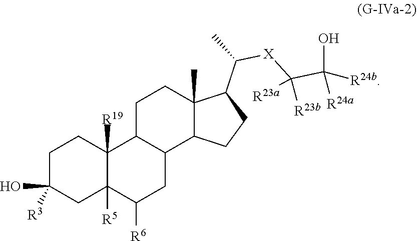

G-17. The compound or pharmaceutically acceptable salt thereof according to any one of embodiments G-1, G-5, G-9, and G-13, wherein each of R^{2a} and R^{2b} is hydrogen.

G-18. The compound or pharmaceutically acceptable salt thereof according to any one of embodiments G-1 to G-16, wherein R³ is unsubstituted C₁₋₆alkyl.

G-19. The compound or pharmaceutically acceptable salt thereof according to embodiment G-18, wherein R³ is —CH₃, —CH₂CH₃, or —CH₂CH₂CH₃.

G-20. The compound or pharmaceutically acceptable salt thereof according to embodiment G-19, wherein R³ is —CH₃.

G-21. The compound or pharmaceutically acceptable salt thereof according to embodiment G-19, wherein R³ is —CH₂CH₃.

G-22. The compound or pharmaceutically acceptable salt thereof according to any one of embodiments G-1, G-5, G-9, and G-13, wherein each of R^{11a} and R^{11b} is hydrogen.

G-23. The compound or pharmaceutically acceptable salt thereof according to any one of embodiments G-1, G-2, G-4 to G-6, G-8 to G-10, and G-12 to G-14, wherein R⁵, when present, is hydrogen.

G-24. The compound or pharmaceutically acceptable salt thereof according to any one of embodiments G-1, G-2, G-4 to G-6, G-8 to G-10, and G-12 to G-14, wherein R⁵, when present, is in the alpha configuration.

G-25. The compound or pharmaceutically acceptable salt thereof according to embodiment G-24, wherein R⁵ is hydrogen.

G-26. The compound or pharmaceutically acceptable salt thereof according to any one of embodiments G-1 to G-16, wherein R⁶ is hydrogen.

G-27. The compound or pharmaceutically acceptable salt thereof according to any one of embodiments G-1 to G-16, wherein R¹⁹ is hydrogen, unsubstituted C₂₋₆alkyl, or C₂₋₆alkyl substituted with OR^{A19}, wherein R^{A19} is substituted or unsubstituted C₁₋₆alkyl.

G-28. The compound or pharmaceutically acceptable salt thereof according to embodiment G-27, wherein R¹⁹ is hydrogen, —OCH₃, —CH₂CH₃, or —CH(CH₃)₂.

G-29. The compound or pharmaceutically acceptable salt thereof according to embodiment G-28, wherein R¹⁹ is hydrogen or —CH₂CH₃.

G-30. The compound or pharmaceutically acceptable salt thereof according to embodiment G-29, wherein R¹⁹ is hydrogen.

G-31. The compound or pharmaceutically acceptable salt thereof according to any one of embodiments G-1 to G-16, wherein X is —(CH₂)ₙ— and n is 1.

G-32. The compound or pharmaceutically acceptable salt thereof according to any one of embodiments G-1 to G-16, wherein X is —(CH₂)ₙ— and n is 2.

G-33. The compound or pharmaceutically acceptable salt thereof according to any one of embodiments G-1 to G-16, wherein each of R^{23a} and R^{23b} is hydrogen.

G-34. The compound or pharmaceutically acceptable salt thereof according to any one of embodiments G-1 to G-16, wherein at least one of R^{23a} and R^{23b} is fluorine.

G-35. The compound or pharmaceutically acceptable salt thereof according to any one of embodiments G-1 to G-16, wherein each of R^{23a} and R^{23b} is fluorine.

G-36. The compound or pharmaceutically acceptable salt thereof according to any one of embodiments G-1 to G-16, wherein R^{24a} is C₁₋₆alkyl substituted with fluorine.

G-37. The compound or pharmaceutically acceptable salt thereof according to any one of embodiments G-1 to G-16, wherein R^{24a} is —CF₃.

G-38. The compound or pharmaceutically acceptable salt thereof according to any one of embodiments G-1 to G-16, wherein R^{24b} is unsubstituted C₁₋₆alkyl.

G-39. The compound or pharmaceutically acceptable salt thereof according to any one of embodiments G-1 to G-16, wherein R^{24b} is —CH₃.

G-40. The compound or pharmaceutically acceptable salt thereof according to any one of embodiments G-1 to G-16, wherein R^{24a} is —CH₃ and R^{24b} is —CH₃.

G-41. The compound or pharmaceutically acceptable salt thereof according to any one of embodiments G-1 to G-16, wherein R^{24a} is —CF₃ and R^{24b} is —CH₃.

G-42. The compound or pharmaceutically acceptable salt thereof according to any one of embodiments G-1 to G-16, wherein R^{24a} is —CH₃ and R^{24b} is —CF₃.

G-43. The compound or pharmaceutically acceptable salt thereof according to any one of embodiments G-1 to G-8, wherein q is 0 and r is 1.

G-44. The compound or pharmaceutically acceptable salt thereof according to any one of embodiments G-1 to G-8, wherein q is 0 and r is 2.

G-45. The compound or pharmaceutically acceptable salt thereof according to any one of embodiments G-1 to G-8, wherein q is 1 and r is 2.

G-46. The compound or pharmaceutically acceptable salt thereof according to any one of embodiments G-1 to G-8, wherein q is 2 and r is 1.

G-47. The compound or pharmaceutically acceptable salt thereof according to any one of embodiments G-1 to G-8, wherein q is 2 and r is 2.

G-48. The compound or pharmaceutically acceptable salt thereof according to any one of embodiments G-1 to G-4 and G-9 to G-12, wherein s is 1.

G-49. The compound or pharmaceutically acceptable salt thereof according to any one of embodiments G-1 to G-4 and G-9 to G-12, wherein s is 2.

G-50. The compound or pharmaceutically acceptable salt thereof according to any one of embodiments G-1 to G-4 and G-9 to G-12, wherein s is 3.

G-51. The compound or pharmaceutically acceptable salt thereof according to any one of embodiments G-1 to G-4 and G-9 to G-12, wherein s is 4.

G-52. The compound or pharmaceutically acceptable salt thereof according to embodiment G-1, wherein the compound of Formula (G-I) is selected from the group consisting of Compounds G-1-G-5 in Table G-1, and pharmaceutically acceptable salts thereof.

G-53. A pharmaceutical composition comprising a compound or pharmaceutically acceptable salt thereof according to any one of embodiments G-1 to G-52, and a pharmaceutically acceptable carrier.

G-54. A method for treating a CNS-related condition in a subject, comprising administering to the subject an effective amount of a compound or pharmaceutically acceptable salt thereof according to any one of embodiments G-1 to G-52, or a pharmaceutical composition according to embodiment G-53.

G-55. The method according to embodiment G-54, wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease), and tinnitus.

G-56. A method of inducing sedation or anesthesia in a subject, comprising administering to the subject an effective amount of a compound or pharmaceutically acceptable salt thereof according to any one of embodiments G-1 to G-52, or a pharmaceutical composition according to embodiment G-53.

G-57. A compound or pharmaceutically acceptable salt thereof according to any one of embodiments G-1 to G-52, or a pharmaceutical composition according to embodiment G-53, for use in treating a CNS-related condition in a subject.

G-58. The compound or pharmaceutically acceptable salt thereof, or pharmaceutical composition for use according to embodiment G-57, wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease), and tinnitus.

G-59. A compound or pharmaceutically acceptable salt thereof according to any one of embodiments G-1 to G-52, or a pharmaceutical composition according to embodiment G-53, for use in inducing sedation or anesthesia in a subject.

G-60. Use of a compound or pharmaceutically acceptable salt thereof according to any one of embodiments G-1 to G-52, or a pharmaceutical composition according to embodiment G-53, for the manufacture of a medicament for treating a CNS-related condition in a subject.

G-61. The use according to embodiment G-60, wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease), and tinnitus.

G-62. Use of a compound or pharmaceutically acceptable salt thereof according to any one of embodiments G-1 to G-52, or a pharmaceutical composition according to embodiment G-53, for the manufacture of a medicament for inducing sedation or anesthesia in a subject.

G-63. A compound of Formula (G-I):

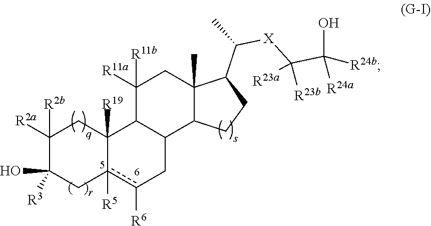

wherein:
   each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein R^{A2} is hydrogen or substituted or unsubstituted C₁₋₆alkyl;
   R³ is substituted or unsubstituted C₁₋₆alkyl, substituted or unsubstituted C₂₋₆alkenyl, or substituted or unsubstituted C₂₋₆alkynyl;
   R⁵ is absent, hydrogen, substituted or unsubstituted C₁₋₆alkyl or halogen;
   R⁶ is hydrogen, substituted or unsubstituted C₁₋₆alkyl, or halogen;
   R¹⁹ is hydrogen, substituted or unsubstituted C₂₋₆alkyl, or OR^{A19}; wherein each instance of R^{A19} is independently substituted or unsubstituted C₁₋₆alkyl;
   R^{11a} is hydrogen or —OR^{A11}, wherein R^{A11} is hydrogen or substituted or unsubstituted C₁₋₆alkyl, and R^{11b} is hydrogen; or R^{11a} and R^{11b} are joined to form an oxo (═O) group;
   X is —(C(R^{X})₂)ₙ— or —O—, wherein R^{X} is hydrogen or fluorine, or one R^{X} group and R^{23b} are joined to form a double bond;
   n is 1, 2 or 3;
   each of R^{23a} and R^{23b} is independently hydrogen or fluorine;
   R^{24a} is selected from the group consisting of substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl; and
   R^{24b} is hydrogen or substituted or unsubstituted C₁₋₆alkyl;
   or R^{24a} and R^{24b}, taken together with the carbon atom to which they are attached, form a substituted or unsubstituted C₃₋₆carbocyclyl;
   q is 0, 1 or 2;
   r is 1 or 2;
   s is 1, 2, 3 or 4; and
   [symbol] represents a single or double bond; provided if the bond between C5 and C6 is a double bond, R⁵ is absent.

G-64. The compound according to embodiment G-63, wherein the compound of Formula (G-I) is a compound of Formula (G-Ia):

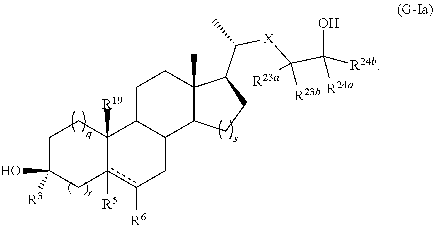

G-65. The compound according to embodiment G-64, wherein the compound of Formula (G-Ia) is a compound of Formula (G-Ia-1):

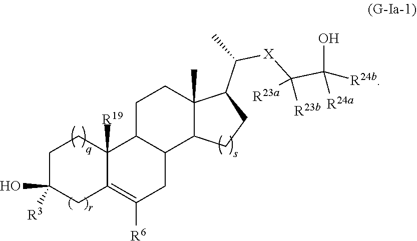

G-66. The compound according to embodiment G-64, wherein the compound of Formula (G-Ia) is a compound of Formula (G-Ia-2):

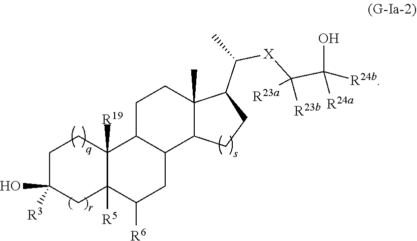

G-67. The compound according to embodiment G-63, wherein the compound of Formula (G-I) is a compound of Formula (G-II):

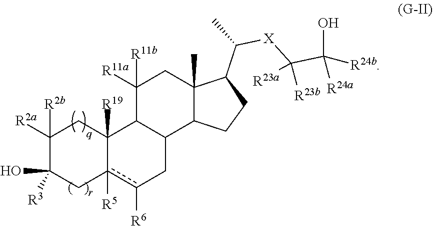

G-68. The compound according to embodiment G-67, wherein the compound of Formula (G-II) is a compound of Formula (G-IIa):

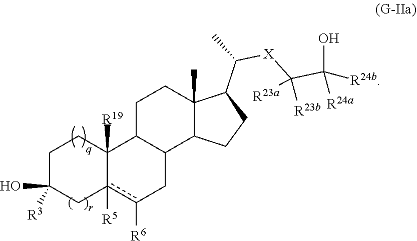

G-69. The compound according to embodiment G-68, wherein the compound of Formula (G-IIa) is a compound of Formula (G-IIa-1):

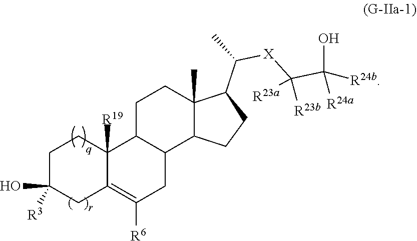

G-70. The compound according to embodiment G-68, wherein the compound of Formula (G-IIa) is a compound of Formula (G-IIa-2):

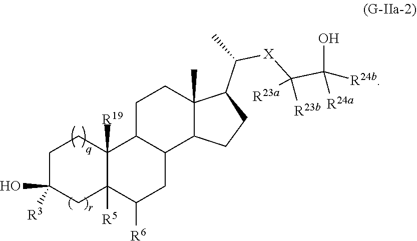

G-71. The compound according to embodiment G-63, wherein the compound of Formula (G-I) is a compound of Formula (G-III):

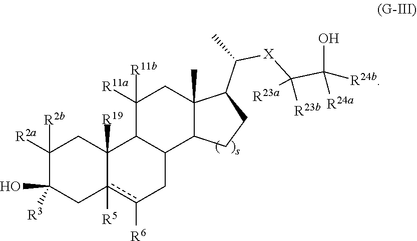

G-72. The compound according to embodiment G-71, wherein the compound of Formula (G-III) is a compound of Formula (G-IIIa):

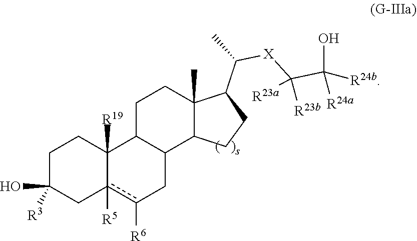

G-73. The compound according to embodiment G-72, wherein the compound of Formula (G-IIIa) is a compound of Formula (G-IIIa-1):

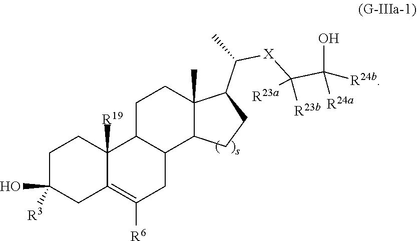

G-74. The compound according to embodiment G-72, wherein the compound of Formula (G-IIIa) is a compound of Formula (G-IIIa-2):

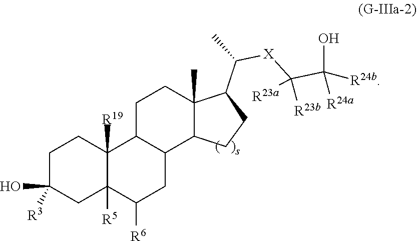

G-75. The compound according to embodiment G-63, wherein the compound of Formula (G-I) is a compound of Formula (G-IV):

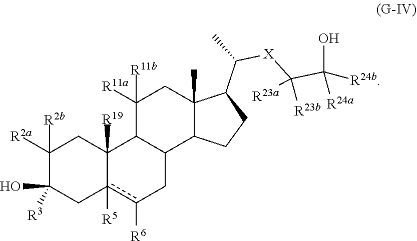

G-76. The compound according to embodiment G-75, wherein the compound of Formula (G-IV) is a compound of Formula (G-IVa):

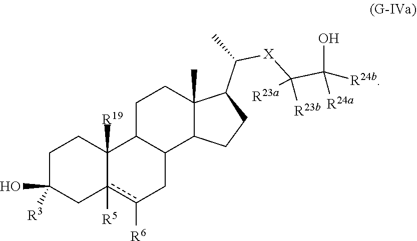

G-77. The compound according to embodiment G-76, wherein the compound of Formula (G-IVa) is a compound of Formula (G-IVa-1):

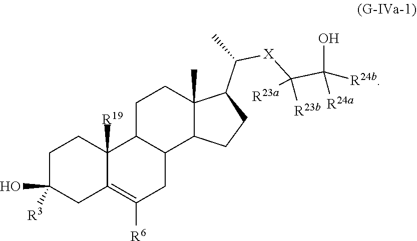

G-78. The compound according to embodiment G-76, wherein the compound of Formula (G-IVa) is a compound of Formula (G-IVa-2):

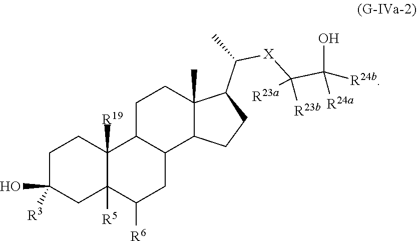

G-79. The compound according to any one of embodiments G-63, G-67, G-71, and G-75, wherein each of R^{2a} and R^{2b} is hydrogen.

G-80. The compound according to any one of embodiments G-63 to G-78, wherein R³ is unsubstituted C₁₋₆alkyl.

G-81. The compound according to embodiment G-80, wherein R³ is —CH₃, —CH₂CH₃, or —CH₂CH₂CH₃.

G-82. The compound according to embodiment G-81, wherein R³ is —CH₃.

G-83. The compound according to embodiment G-81, wherein R³ is —CH₂CH₃.

G-84. The compound according to any one of embodiments G-63, G-67, G-71, and G-75, wherein each of R^{11a} and R^{11b} is hydrogen.

G-85. The compound according to any one of embodiments G-63, G-64, G-66 to G-68, G-70 to G-72, and G-74 to G-76, wherein R⁵, when present, is hydrogen.

G-86. The compound according to any one of embodiments G-63, G-64, G-66 to G-68, G-70 to G-72, and G-74 to G-76, wherein R⁵, when present, is in the alpha configuration.

G-87. The compound thereof according to embodiment G-86, wherein R⁵ is hydrogen.

G-88. The compound according to any one of embodiments G-63 to G-78, wherein R⁶ is hydrogen.

G-89. The compound according to any one of embodiments G-63 to G-78, wherein R¹⁹ is hydrogen, unsubstituted C₂₋₆alkyl, or C₂₋₆alkyl substituted with OR^{A19} wherein R^{A19} is substituted or unsubstituted C₁₋₆alkyl.

G-90. The compound according to embodiment G-89, wherein R¹⁹ is hydrogen, —OCH₃, —CH₂CH₃, or —CH(CH₃)₂.

G-91. The compound according to embodiment G-90, wherein R¹⁹ is hydrogen or —CH₂CH₃.

G-92. The compound according to embodiment G-91, wherein R¹⁹ is hydrogen.

G-93. The compound according to any one of embodiments G-63 to G-78, wherein X is —(CH₂)ₙ— and n is 1.

G-94. The compound according to any one of embodiments G-63 to G-78, wherein X is —(CH₂)ₙ— and n is 2.

G-95. The compound according to any one of embodiments G-63 to G-78, wherein each of R^{23a} and R^{23b} is hydrogen.

G-96. The compound according to any one of embodiments G-63 to G-78, wherein at least one of R^{23a} and R^{23b} is fluorine.

G-97. The compound according to any one of embodiments G-63 to G-78, wherein each of R^{23a} and R^{23b} is fluorine.

G-98. The compound according to any one of embodiments G-63 to G-78, wherein R^{24a} is C₁₋₆alkyl substituted with fluorine.

G-99. The compound according to any one of embodiments G-63 to G-78, wherein R^{24a} is —CF₃.

G-100. The compound according to any one of embodiments G-63 to G-78, wherein R^{24b} is unsubstituted C₁₋₆alkyl.

G-101. The compound according to any one of embodiments G-63 to G-78, wherein R^{24b} is —CH₃.

G-102. The compound according to any one of embodiments G-63 to G-78, wherein R^{24a} is —CH₃ and R^{24b} is —CH₃.

G-103. The compound according to any one of embodiments G-63 to G-78, wherein R^{24a} is —CF₃ and R^{24b} is —CH₃.

G-104. The compound according to any one of embodiments G-63 to G-78, wherein R^{24a} is —CH₃ and R^{24b} is —CF₃.

G-105. The compound according to any one of embodiments G-63 to G-70, wherein q is 0 and r is 1.

G-106. The compound according to any one of embodiments G-63 to G-70, wherein q is 0 and r is 2.

G-107. The compound according to any one of embodiments G-63 to G-70, wherein q is 1 and r is 2.

G-108. The compound according to any one of embodiments G-63 to G-70, wherein q is 2 and r is 1.

G-109. The compound according to any one of embodiments G-63 to G-70, wherein q is 2 and r is 2.

G-110. The compound according to any one of embodiments G-63 to G-66 and G-71 to G-74, wherein s is 1.

G-111. The compound according to any one of embodiments G-63 to G-66 and G-71 to G-74, wherein s is 2.

G-112. The compound according to any one of embodiments G-63 to G-66 and G-71 to G-74, wherein s is 3.

G-113. The compound according to any one of embodiments G-63 to G-66 and G-71 to G-74, wherein s is 4.

G-114. The compound according to embodiment G-63, wherein the compound of Formula (G-I) is selected from the group consisting of Compounds G-1-G-5 of Table G-1.

G-115. A pharmaceutical composition comprising a compound according to any one of embodiments G-63 to G-114, and a pharmaceutically acceptable carrier.

G-116. A method for treating a CNS-related condition in a subject, comprising administering to the subject an effective amount of a compound according to any one of embodiments G-63 to G-114, or a pharmaceutical composition according to embodiment G-115.

G-117. The method according to embodiment G-116, wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease), and tinnitus.

G-118. A method of inducing sedation or anesthesia in a subject, comprising administering to the subject an effective amount of a compound according to any one of embodiments G-63 to G-114, or a pharmaceutical composition according to embodiment G-115.

G-119. A compound according to any one of embodiments G-63 to G-114, or a pharmaceutical composition according to embodiment G-115, for use in treating a CNS-related condition in a subject.

G-120. The compound, or pharmaceutical composition for use according to embodiment G-119, wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease), and tinnitus.

G-121. A compound according to any one of embodiments G-63 to G-114, or a pharmaceutical composition according to embodiment G-115, for use in inducing sedation or anesthesia in a subject.

G-122. Use of a compound according to any one of embodiments G-63 to G-114, or a pharmaceutical composition according to embodiment G-115, for the manufacture of a medicament for treating a CNS-related condition in a subject.

G-123. The use according to embodiment G-122, wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease), and tinnitus.

G-124. Use of a compound according to any one of embodiments G-63 to G-114, or a pharmaceutical composition according to embodiment G-115, for the manufacture of a medicament for inducing sedation or anesthesia in a subject.

H-1. A compound of Formula (H-I):

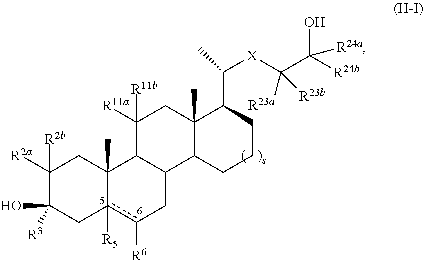

or a pharmaceutically acceptable salt thereof;
wherein:
   each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein R^{A2} is hydrogen or substituted or unsubstituted C₁₋₆alkyl;
   R³ is substituted or unsubstituted C₁₋₆alkyl, substituted or unsubstituted C₂₋₆ alkenyl, substituted or unsubstituted C₂₋₆alkynyl, or substituted or unsubstituted C₃₋₁₀carbocyclyl;
   R⁵ is absent, hydrogen, halogen, or substituted or unsubstituted C₁₋₆alkyl
   R⁶ is hydrogen, substituted or unsubstituted C₁₋₆alkyl, or halogen;
   R^{11a} is hydrogen or —OR^{A11}, wherein R^{A11} is hydrogen or substituted or unsubstituted alkyl, and R^{11b} is hydrogen; or R^{11a} and R^{11b} are joined to form an oxo (═O) group;
   X is —(C(R^{X})₂)ₙ— or —O—, wherein R^{X} is hydrogen or fluorine, or one R^{X} group and R^{23b} are joined to form a double bond;
   n is 1, 2 or 3;
   each of R^{23a} and R^{23b} is independently hydrogen or fluorine;
   R^{24a} is selected from the group consisting of substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, and substituted or unsubstituted heteroaryl group; and
   R^{24b} is hydrogen or substituted or unsubstituted alkyl;
   or R^{24a} and R^{24b}, taken together with the carbon atom to which they are attached, form a substituted or unsubstituted C₃₋₆carbocyclyl or substituted or unsubstituted 3- to 10-membered heterocyclyl;
   s is 1, 2 or 3; and
   [symbol] represents a single or double bond, provided if the bond between C5 and C6 is a double bond, R⁵ is absent.

H-2. The compound or pharmaceutically acceptable salt thereof according to embodiment H-1, wherein the compound of Formula (H-I) is a compound of Formula (H-Ia):

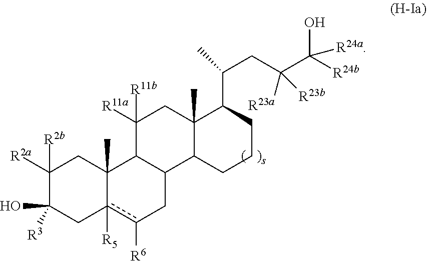

H-3. The compound or pharmaceutically acceptable salt thereof according to embodiment H-2, wherein the compound of Formula (H-Ia) is a compound of Formula (H-Ia-1):

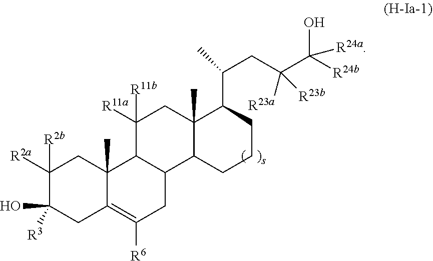

H-4. The compound or pharmaceutically acceptable salt thereof according to embodiment H-3, wherein the compound of Formula (H-Ia) is a compound of Formula (H-Ia-1i) or Formula (H-Ia-1ii):

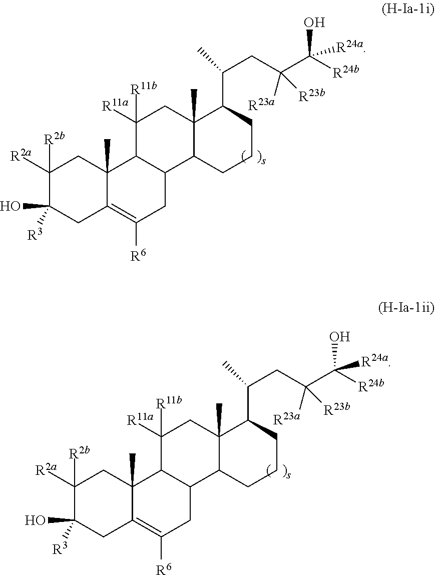

H-5. The compound or pharmaceutically acceptable salt thereof according to embodiment H-2, wherein the compound of Formula (H-Ia) is a compound of Formula (H-Ia-2):

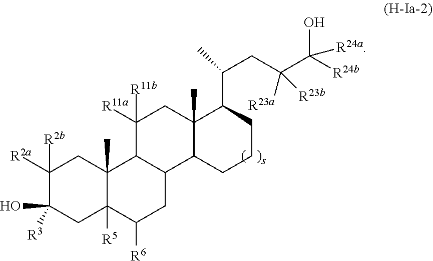

H-6. The compound or pharmaceutically acceptable salt thereof according to embodiment H-5, wherein the compound of Formula (H-Ia-2) is a compound of Formula (H-Ia-2i) or Formula (H-Ia-2ii):

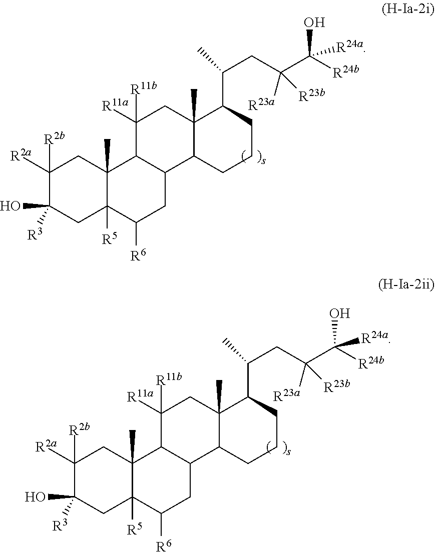

H-7. The compound or pharmaceutically acceptable salt thereof according to embodiment H-6, wherein the compound of Formula (H-Ia-2i) is a compound of Formula (H-Ia-2i-a):

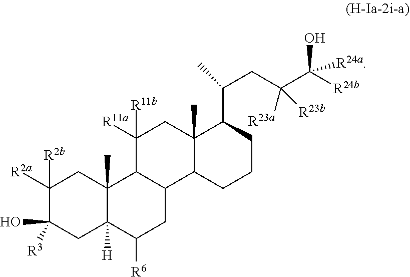

H-8. The compound or pharmaceutically acceptable salt thereof according to embodiment H-6, wherein the compound of Formula (H-Ia-2ii) is a compound of Formula (H-Ia-2ii-a):

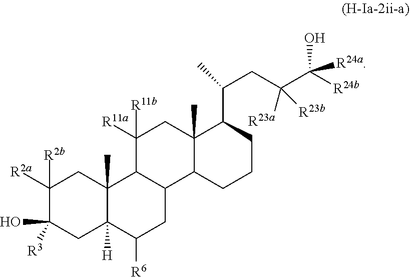

H-9. The compound or pharmaceutically acceptable salt thereof according to embodiment H-1, wherein the compound of Formula (H-I) is a compound of Formula (H-Ib):

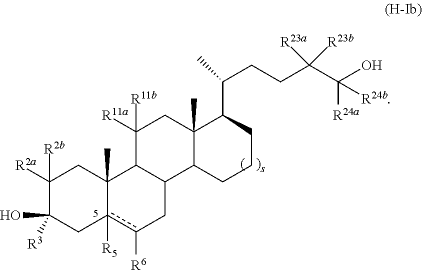

H-10. The compound or pharmaceutically acceptable salt thereof according to embodiment H-9, wherein the compound of Formula (H-Ib) is a compound of Formula (H-Ib-1):

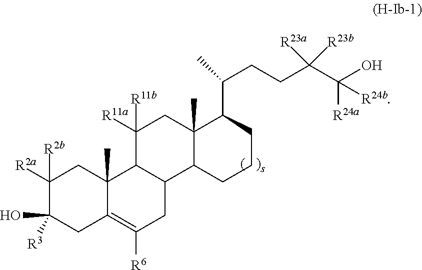

H-11. The compound or pharmaceutically acceptable salt thereof according to embodiment H-10, wherein the compound of Formula (H-Ib-1) is a compound of Formula (H-Ib-1i) or Formula (H-Ib-1ii):

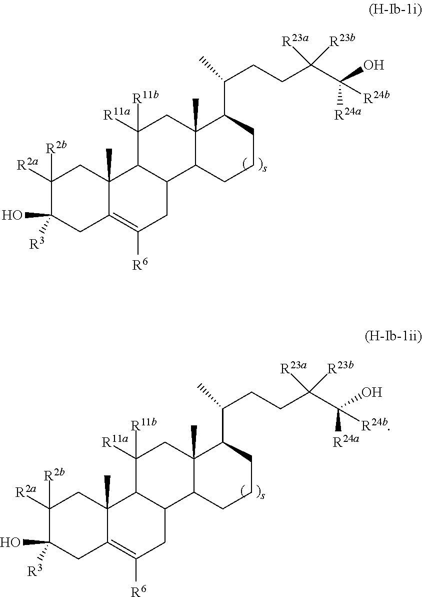

H-12. The compound or pharmaceutically acceptable salt thereof according to embodiment H-9, wherein the compound of Formula (H-Ib) is a compound of Formula (H-Ib-2):

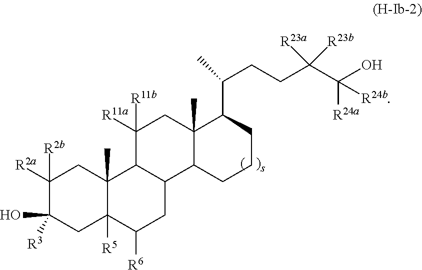

H-13. The compound or pharmaceutically acceptable salt thereof according to embodiment H-12, wherein the compound of Formula (H-Ib-2) is a compound of Formula (H-Ib-2i) or Formula (H-Ib-2ii):

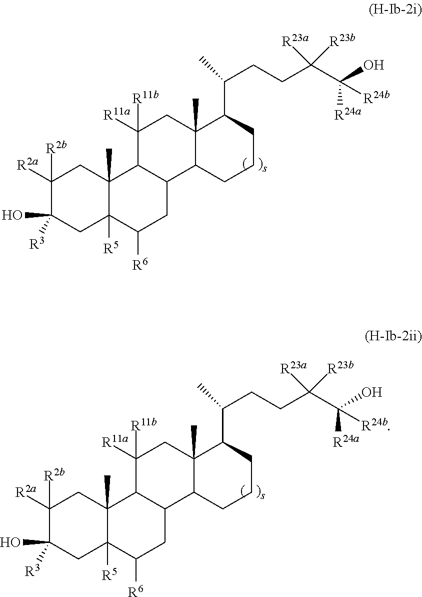

H-14. The compound or pharmaceutically acceptable salt thereof according to embodiment H-13, wherein the compound of Formula (H-Ib-2i) is a compound of Formula (H-Ib-2i-a):

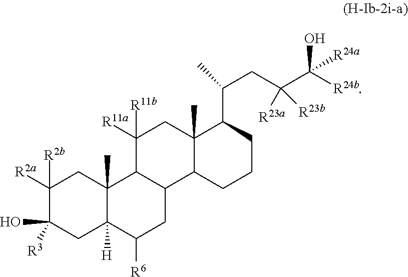

H-15. The compound or pharmaceutically acceptable salt thereof according to embodiment H-13, wherein the compound of Formula (H-Ib-2ii) is a compound of Formula (H-Ib-2ii-a):

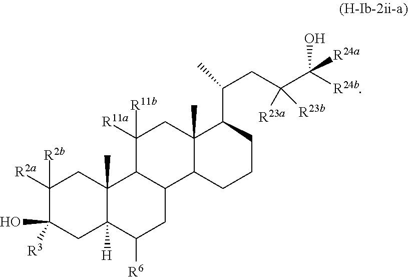

H-16. The compound or pharmaceutically acceptable salt thereof according to any one of embodiments H-1 to H-15, wherein each of R^{2a} and R^{2b} is hydrogen.

H-17. The compound or pharmaceutically acceptable salt thereof according to any one of embodiments H-1 to H-15, wherein R³ is unsubstituted C₁₋₆alkyl.

H-18. The compound or pharmaceutically acceptable salt thereof according to embodiment H-17, wherein R³ is —CH₃, —CH₂CH₃, or —CH₂CH₂CH₃.

H-19. The compound or pharmaceutically acceptable salt thereof according to embodiment H-18, wherein R³ is —CH₃.

H-20. The compound or pharmaceutically acceptable salt thereof according to embodiment H-18, wherein R³ is —CH₂CH₃.

H-21. The compound or pharmaceutically acceptable salt thereof according to any one of embodiments H-1 to H-15, wherein R³ is substituted C₁₋₆alkyl.

H-22. The compound or pharmaceutically acceptable salt thereof according to embodiment H-21, wherein R³ is —CF₃.

H-23. The compound or pharmaceutically acceptable salt thereof according to any one of embodiments H-1 to H-15, wherein each of R^{11a} and R^{11b} is hydrogen.

H-24. The compound or pharmaceutically acceptable salt thereof according to any one of embodiments H-1, H-2, H-5, H-6, H-9, H-12 and H-13, wherein R⁵, when present, is hydrogen.

H-25. The compound or pharmaceutically acceptable salt thereof according to any one of embodiments H-1, H-2, H-5, H-6, H-9, H-12 and H-13, wherein R⁵, when present, is in the alpha configuration.

H-26. The compound or pharmaceutically acceptable salt thereof according to any one of embodiments H-1 to H-15, wherein R⁶ is hydrogen.

H-27. The compound or pharmaceutically acceptable salt thereof according to any one of embodiments H-1 to H-15, wherein each of R^{23a} and R^{23b} is hydrogen.

H-28. The compound or pharmaceutically acceptable salt thereof according to any one of embodiments H-1 to H-15, wherein at least one of R^{23a} and R^{23b} is fluorine.

H-29. The compound or pharmaceutically acceptable salt thereof according to any one of embodiments H-1 to H-15, wherein each of R^{23a} and R^{23b} is fluorine.

H-30. The compound or pharmaceutically acceptable salt thereof according to any one of embodiments H-1 to H-15, wherein R^{24a} is substituted C₁₋₆alkyl.

H-31. The compound or pharmaceutically acceptable salt thereof according to embodiment H-30, wherein R^{24a} is C₁₋₆alkyl substituted with fluorine.

H-32. The compound or pharmaceutically acceptable salt thereof according to embodiment H-31, wherein R^{24a} is —CF₃.

H-33. The compound or pharmaceutically acceptable salt thereof according to any one of embodiments H-1 to H-15, wherein R^{24a} is unsubstituted C₁₋₆alkyl.

H-34. The compound or pharmaceutically acceptable salt thereof according to embodiment H-33, wherein R^{24a} is —CH₃ or —CH(CH₃)₂.

H-35. The compound or pharmaceutically acceptable salt thereof according to embodiment H-34, wherein R^{24a} is —CH₃.

H-36. The compound or pharmaceutically acceptable salt thereof according to embodiment H-34, wherein R^{24a} is —CH(CH₃)₂.

H-37. The compound or pharmaceutically acceptable salt thereof according to any one of embodiments H-1 to H-15, wherein R^{24b} is H.

H-38. The compound or pharmaceutically acceptable salt thereof according to any one of embodiments H-1 to H-15, wherein R^{24b} is substituted C₁₋₆alkyl.

H-39. The compound or pharmaceutically acceptable salt thereof according to embodiment H-38, wherein R^{24b} is C₁₋₆alkyl substituted with fluorine.

H-40. The compound or pharmaceutically acceptable salt thereof according to embodiment H-39, wherein R^{24b} is —CF₃.

H-41. The compound or pharmaceutically acceptable salt thereof according to any one of embodiments H-1 to H-15, wherein R^{24b} is unsubstituted C₁₋₆alkyl.

H-42. The compound or pharmaceutically acceptable salt thereof according to embodiment H-41, wherein R^{24b} is —CH₃.

H-43. The compound or pharmaceutically acceptable salt thereof according to any one of embodiments H-1 to H-15, wherein R^{24a} is —CF₃ and R^{24b} is —CH₃.

H-44. The compound or pharmaceutically acceptable salt thereof according to any one of embodiments H-1 to H-15, wherein R^{24a} is —CH(CH₃)₂ and R^{24b} is —H.

H-45. The compound or pharmaceutically acceptable salt thereof according to any one of embodiments H-1 to H-15, wherein each of R^{24a} and R^{24b} is —CH₃.

H-46. The compound or pharmaceutically acceptable salt thereof according to any one of embodiments H-1 to H-15, wherein R^{24a} and R^{24b}, together with the carbon atom to which they are attached, form a substituted C₃₋₆carbocyclyl.

H-47. The compound or a pharmaceutically acceptable salt thereof according to embodiment H-46, wherein the substituted C₃₋₆carbocyclyl is substituted with one or more C₁₋₆alkyl.

H-48. The compound or a pharmaceutically acceptable salt thereof according to any one of embodiments H-1 to H-6 or H-9 to H-13, wherein s is 1.

H-49. The compound or a pharmaceutically acceptable salt thereof according to any one of embodiments H-1 to H-6 or H-9 to H-13, wherein s is 2.

H-50. The compound or a pharmaceutically acceptable salt thereof according to any one of embodiments H-1 to H-6 or H-9 to H-13, wherein s is 3.

H-51. The compound according to embodiment H-1, wherein the compound is selected from the group consisting of H-1-H-11 of Table H-1, and pharmaceutically acceptable salts thereof.

H-52. The compound according to embodiment H-1, wherein the compound is selected from the group consisting of Compounds H-1-H4 of Table H-1, or pharmaceutically acceptable salts thereof.

H-53. A pharmaceutical composition comprising a compound or pharmaceutically acceptable salt thereof according to any one of embodiments H-1 to H-52, and a pharmaceutically acceptable carrier.

H-54. A method for treating a CNS-related condition in a subject comprising administering to the subject an effective amount of a compound or pharmaceutically acceptable salt thereof according to any one of embodiments H-1 to H-52, or a pharmaceutical composition according to embodiment H-53.

H-55. The method according to embodiment H-54, wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease), and tinnitus.

H-56. A method of inducing sedation or anesthesia in a subject, comprising administering to the subject an effective amount of a compound or pharmaceutically acceptable salt thereof according to any one of embodiments H-1 to H-52, or a pharmaceutical composition according to embodiment H-53.

H-57. A compound or pharmaceutically acceptable salt thereof according to any one of embodiments H-1 to H-52, or a pharmaceutical composition according to embodiment H-53, for use in treating a CNS-related condition in a subject.

H-58. The compound or pharmaceutically acceptable salt thereof, or pharmaceutical composition for use according to embodiment H-57, wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease), and tinnitus.

H-59. A compound or pharmaceutically acceptable salt thereof according to any one of embodiments H-1 to H-52, or a pharmaceutical composition according to embodiment H-53, for use in inducing sedation or anesthesia in a subject.

H-60. Use of a compound or pharmaceutically acceptable salt thereof according to any one of embodiments H-1 to H-52, or a pharmaceutical composition according to embodiment H-53, for the manufacture of a medicament for treating a CNS-related condition in a subject.

H-61. The use according to embodiment H-60, wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease), and tinnitus.

H-62. Use of a compound or pharmaceutically acceptable salt thereof according to any one of embodiments H-1 to H-52, or a pharmaceutical composition according to embodiment H-53, for the manufacture of a medicament for inducing sedation or anesthesia in a subject.

H-63. A compound of Formula (H-I):

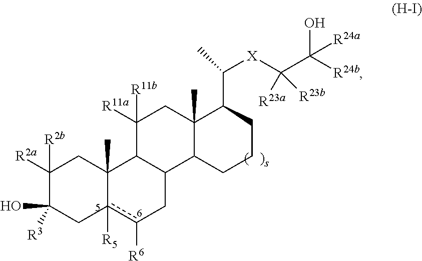

wherein:
   each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein R^{A2} is hydrogen or substituted or unsubstituted C₁₋₆alkyl;
   R³ is substituted or unsubstituted C₁₋₆alkyl, substituted or unsubstituted C₂₋₆ alkenyl, substituted or unsubstituted C₂₋₆alkynyl, or substituted or unsubstituted C₃₋₁₀ carbocyclyl;
   R⁵ is absent, hydrogen, halogen, or substituted or unsubstituted C₁₋₆alkyl
   R⁶ is hydrogen, substituted or unsubstituted C₁₋₆alkyl, or halogen;
   R^{11a} is hydrogen or —OR^{A11}, wherein R^{A11} is hydrogen or substituted or unsubstituted alkyl, and R^{11b} is hydrogen; or R^{11a} and R^{11b} are joined to form an oxo (═O) group;
   X is —(C(R^{X})₂)ₙ— or —O—, wherein R^{X} is hydrogen or fluorine, or one R^{X} group and R^{23b} are joined to form a double bond;
   n is 1, 2 or 3;
   each of R^{23a} and R^{23b} is independently hydrogen or fluorine;
   R^{24a} is selected from the group consisting of substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, and substituted or unsubstituted heteroaryl group; and
   R^{24b} is hydrogen or substituted or unsubstituted alkyl;
   or R^{24a} and R^{24b}, taken together with the carbon atom to which they are attached, form a substituted or unsubstituted C₃₋₆carbocyclyl, or substituted or unsubstituted 3- to 10-membered heterocyclyl;
   s is 1, 2 or 3; and
   [symbol] represents a single or double bond, provided if the bond between C5 and C6 is a double bond, R⁵ is absent.

H-64. The compound according to embodiment H-63, wherein the compound of Formula (H-I) is a compound of Formula (H-Ia):

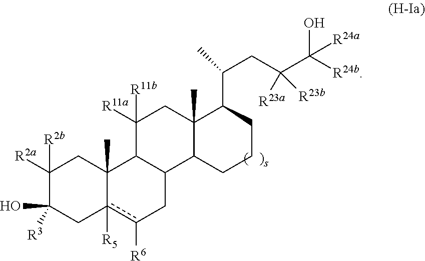

H-65. The compound according to embodiment H-64, wherein the compound of Formula (H-Ia) is a compound of Formula (H-Ia-1):

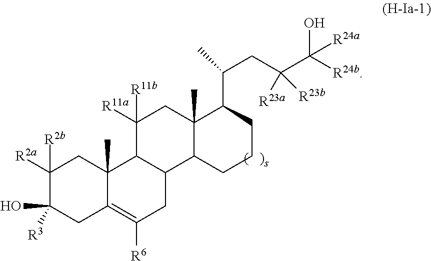

H-66. The compound according to embodiment H-65, wherein the compound of Formula (H-Ia) is a compound of Formula (H-Ia-1i) or Formula (H-Ia-1ii):

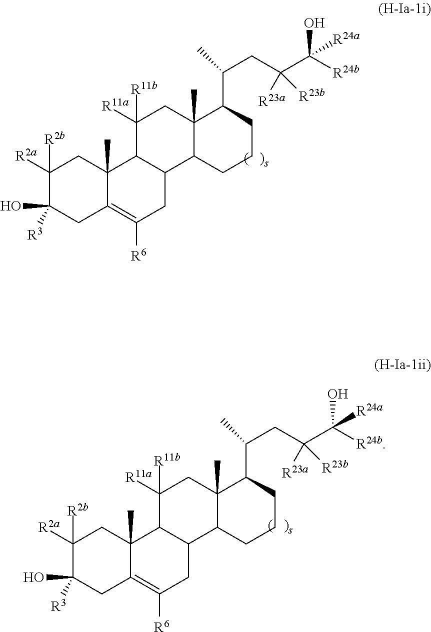

H-67. The compound according to embodiment H-64, wherein the compound of Formula (H-Ia) is a compound of Formula (H-Ia-2):

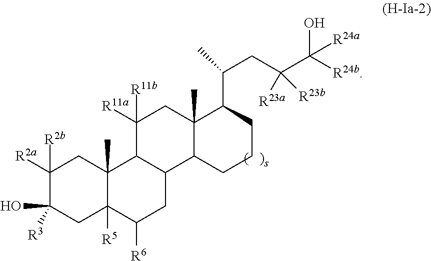

H-68. The compound according to embodiment H-67, wherein the compound of Formula (H-La-2) is a compound of Formula (H-Ia-2i) or Formula (H-Ia-2ii):

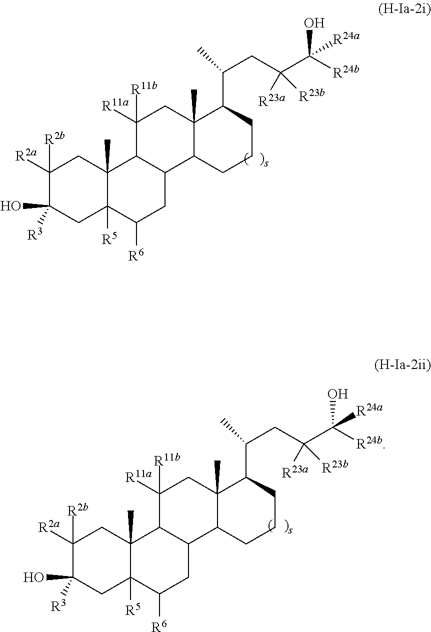

H-69. The compound according to embodiment H-68, wherein the compound of Formula (H-Ia-2i) is a compound of Formula (H-Ia-2i-a):

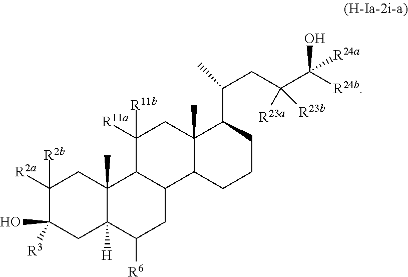

H-70. The compound according to embodiment H-68, wherein the compound of Formula (H-Ia-2ii) is a compound of Formula (H-Ia-2ii-a):

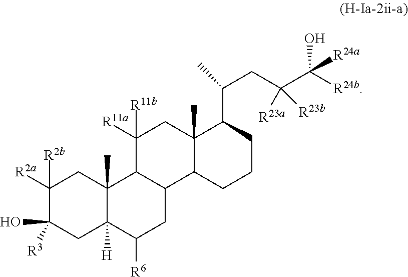

H-71. The compound according to embodiment H-63, wherein the compound of Formula (H-I) is a compound of Formula (H-Ib):

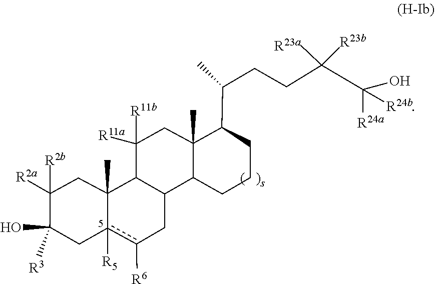

H-72. The compound according to embodiment H-71, wherein the compound of Formula (H-1b) is a compound of Formula (H-Ib-1):

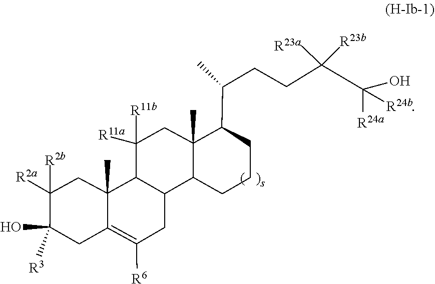

H-73. The compound according to embodiment H-72, wherein the compound of Formula (H-Ib-1) is a compound of Formula (H-Ib-1i) or Formula (H-Ib-1ii):

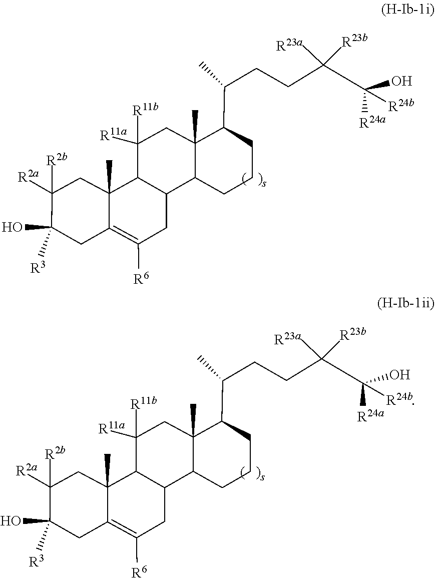

H-74. The compound according to embodiment H-71, wherein the compound of Formula (H-Ib) is a compound of Formula (H-Ib-2):

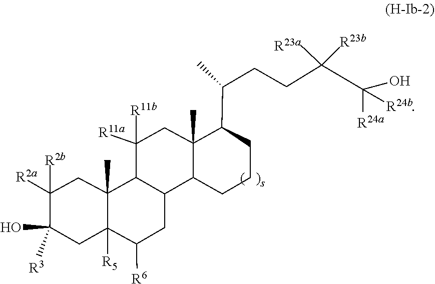

H-75. The compound according to embodiment H-74, wherein the compound of Formula (H-Ib-2) is a compound of Formula (H-Ib-2i) or Formula (H-Ib-2ii):

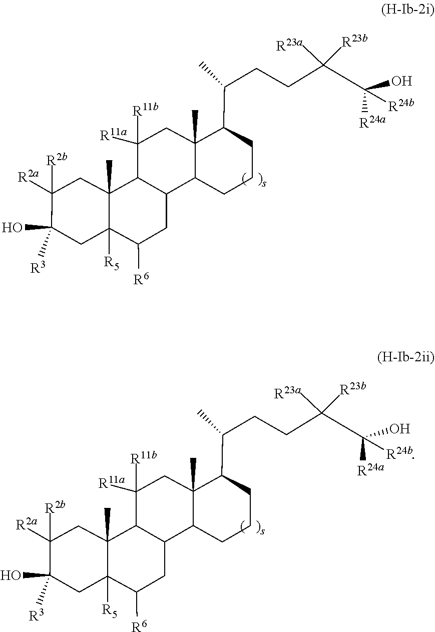

H-76. The compound according to embodiment H-75, wherein the compound of Formula (H-Ib-2i) is a compound of Formula (H-Ib-2i-a):

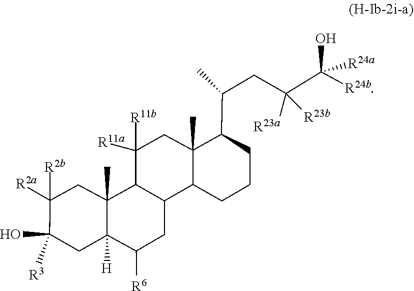

H-77. The compound according to embodiment H-75, wherein the compound of Formula (H-Ib-2ii) is a compound of Formula (H-Ib-2ii-a):

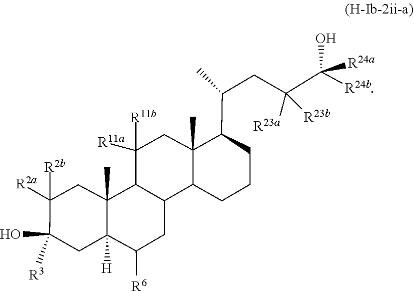

H-78. The compound according to any one of embodiments H-63 to H-77, wherein each of R^{2a} and R^{2b} is hydrogen.

H-79. The compound according to any one of embodiments H-63 to H-77, wherein R³ is unsubstituted C₁₋₆alkyl.

H-80. The compound according to embodiment H-79, wherein R³ is —CH₃, —CH₂CH₃, or —CH₂CH₂CH₃.

H-81. The compound according to embodiment H-80, wherein R³ is —CH₃.

H-82. The compound according to embodiment H-80, wherein R³ is —CH₂CH₃.

H-83. The compound according to any one of embodiments H-63 to H-77, wherein R³ is substituted C₁₋₆alkyl.

H-84. The compound according to embodiment H-83, wherein R³ is —CF₃.

H-85. The compound according to any one of embodiments H-63 to H-77, wherein each of R^{11a} and R^{11b} is hydrogen.

H-86. The compound according to any one of embodiments H-63, H-64, H-67, H-68, H-71, H-74, and H-75, wherein R⁵, when present, is hydrogen.

H-87. The compound according to any one of embodiments H-63, H-64, H-67, H-68, H-71, H-74, and H-75, wherein R⁵, when present, is in the alpha configuration.

H-88. The compound according to any one of embodiments H-63 to H-77, wherein R⁶ is hydrogen.

H-89. The compound according to any one of embodiments H-63 to H-77, wherein each of R^{23a} and R^{23b} is hydrogen.

H-90. The compound according to any one of embodiments H-63 to H-77, wherein at least one of R^{23a} and R^{23b} is fluorine.

H-91. The compound according to any one of embodiments H-63 to H-77, wherein each of R^{23a} and R^{23b} is fluorine.

H-92. The compound according to any one of embodiments H-63 to H-77, wherein R^{24a} is substituted C₁₋₆alkyl.

H-93. The compound according to embodiment H-92, wherein R^{24a} is C1-6alkyl substituted with fluorine.

H-94. The compound according to embodiment H-92, wherein R^{24a} is —CF₃.

H-95. The compound according to any one of embodiments H-63 to H-77, wherein R^{24a} is unsubstituted C₁₋₆alkyl.

H-96. The compound according to embodiment H-95, wherein R^{24a} is —CH₃ or —CH(CH₃)₂.

H-97. The compound according to embodiment H-96, wherein R^{24a} is —CH₃.

H-98. The compound according to embodiment H-96, wherein R^{24a} is —CH(CH₃)₂.

H-99. The compound according to any one of embodiments H-63 to H-77, wherein R^{24b} is H.

H-100. The compound according to any one of embodiments H-63 to H-77, wherein R^{24b} is substituted C₁₋₆alkyl.

H-101. The compound according to embodiment H-100, wherein R^{24b} is C1-6alkyl substituted with fluorine.

H-102. The compound according to embodiment H-101, wherein R^{24b} is —CF₃.

H-103. The compound according to any one of embodiments 63-77, wherein R^{24b} is unsubstituted C₁₋₆alkyl.

H-104. The compound according to embodiment 103, wherein R^{24b} is —CH₃.

H-105. The compound according to any one of embodiments H-63 to H-77, wherein R^{24a} is —CF₃ and R^{24b} is —CH₃.

H-106. The compound according to anyone of embodiments H-63 to H-77, wherein R^{24a} is —CH(CH₃)₂ and R^{24b} is —H.

H-107. The compound according to any one of embodiments H-63 to H-77, wherein each of R^{24a} and R^{24b} is —CH₃.

H-108. The compound according to any of the embodiments H-63 to H-77, wherein R^{24a} and R^{24b}, together with the carbon atom to which they are attached, form a substituted C₃₋₆carbocyclyl.

H-109. The compound according to embodiment H-108, wherein the substituted C₃₋₆carbocyclyl is substituted with one or more C₁₋₆alkyl.

H-110. The compound according to any of the embodiments H-63 to H-68 or H-71 to H-75, wherein s is 1.

H-111. The compound according to any of the embodiments H-63 to H-68 or H-71 to H-75, wherein s is 2.

H-112. The compound according to any of the embodiments H-63 to H-68 or H-71 to H-75, wherein s is 3.

H-113. The compound according to embodiment H-63, wherein the compound is selected from the group consisting of Compounds H-1-H-11 of Table H-1.

H-114. The compound according to embodiment H-63, wherein the compound is selected from the group consisting of Compounds H-1-H-4 of Table H-1.

H-115. A pharmaceutical composition comprising a compound according to any one of embodiments H-63 to H-114, and a pharmaceutically acceptable carrier.

H-116. A method for treating a CNS-related condition in a subject comprising administering to the subject an effective amount of a compound according to any one of embodiments H-63 to H-114, or a pharmaceutical composition according to embodiment H-115.

H-117. The method according to embodiment H-116, wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease), and tinnitus.

H-118. A method of inducing sedation or anesthesia in a subject, comprising administering to the subject an effective amount of a compound according to any one of embodiments H-63 to H-114, or a pharmaceutical composition according to embodiment H-115.

H-119. A compound according to any one of embodiments H-63 to H-114, or a pharmaceutical composition according to embodiment H-115, for use in treating a CNS-related condition in a subject.

H-120. The compound or pharmaceutical composition for use according to embodiment H-119, wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease), and tinnitus.

H-121. A compound according to any one of embodiments H-63 to H-114, or a pharmaceutical composition according to embodiment H-115, for use in inducing sedation or anesthesia in a subject.

H-122. Use of a compound according to any one of embodiments H-63 to H-114, or a pharmaceutical composition according to embodiment H-115, for the manufacture of a medicament for treating a CNS-related condition in a subject.

H-123. The use according to embodiment H-122, wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease), and tinnitus.

H-124. Use of a compound according to any one of embodiments H-63 to H-114, or a pharmaceutical composition according to embodiment H-115, for the manufacture of a medicament for inducing sedation or anesthesia in a subject.

What is claimed:

1. A compound of Formula (A-I): 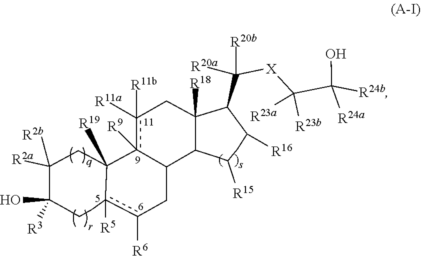
or a pharmaceutically acceptable salt thereof;
wherein:
each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein R^{A2} is hydrogen or substituted or unsubstituted C₁₋₆alkyl;
R³ is substituted or unsubstituted C₁₋₆alkyl, substituted or unsubstituted C₂₋₆ alkenyl, substituted or unsubstituted C₂₋₆alkynyl, or substituted or unsubstituted C₃₋₁₀carbocyclyl;
R⁵ is absent, hydrogen, halogen, or substituted or unsubstituted C₁₋₆alkyl;
R⁶ is hydrogen, halogen, or substituted or unsubstituted C₁₋₆alkyl;
R⁹ is absent or hydrogen; or
R⁹ and R^{11a} are taken together with the carbon atoms to which they are attached to form a substituted or unsubstituted C₃₋₆carbocyclyl ring;
R^{11a} is hydrogen, halogen, substituted or unsubstituted C₁₋₆alkyl, or —OR^{A11}, wherein R^{A11} is hydrogen or substituted or unsubstituted C₁₋₆alkyl;
R^{11b} is absent or hydrogen; or
R^{11a} and R^{11b} are joined to form an oxo (═O) group;
each instance of R¹⁵ is hydrogen, halogen, —OH, —NH₂, substituted or unsubstituted C₁₋₆alkyl, substituted or unsubstituted C₂₋₆alkenyl, substituted or unsubstituted C₂₋₆alkynyl, substituted or unsubstituted C₁₋₆alkoxy, substituted or unsubstituted C₃₋₆carbocyclyl, or substituted or unsubstituted 3- to 10-membered heterocyclyl;
R¹⁶ is hydrogen, halogen, —OH, —NH₂, substituted or unsubstituted C₁₋₆alkyl, substituted or unsubstituted C₂₋₆alkenyl, substituted or unsubstituted C₂₋₆alkynyl, substituted or unsubstituted C₁₋₆alkoxy, substituted or unsubstituted C₃₋₆carbocyclyl, or substituted or unsubstituted 3- to 10-membered heterocyclyl; or
R¹⁵ and R¹⁶ are taken together with the carbon atoms to which they are attached, to form a substituted or unsubstituted C₃₋₆carbocyclyl;
R¹⁸ is hydrogen, substituted or unsubstituted C₁₋₆alkyl, or OR^{A18}, wherein each instance of R^{A18} is independently substituted or unsubstituted C₁₋₆alkyl;
R¹⁹ is hydrogen, substituted or unsubstituted C₁₋₆alkyl, or OR^{A19}; wherein each instance of R^{A19} is independently substituted or unsubstituted C₁₋₆alkyl;
R^{20a} is halogen, cyano, substituted C₁₋₆alkyl, unsubstituted C₂₋₆alkyl, substituted or unsubstituted C₁₋₆alkoxy, or substituted or unsubstituted C₃₋₆carbocyclyl;
R^{20b} is hydrogen, halogen, cyano, substituted or unsubstituted C₁₋₆alkyl, substituted or unsubstituted C₁₋₆alkoxy, or substituted or unsubstituted C₃₋₆carbocyclyl;
or R^{20a} and R^{20b} are both simultaneously —CH₃;
X is —(C(R^{X})₂)ₙ— or —O—, wherein R^{X} is hydrogen or fluorine, or one R^{X} group and R^{23b} are joined to form a double bond;
n is 1, 2 or 3;
each instance of R^{23a} and R^{23b} is independently hydrogen or fluorine;
R^{24a} is selected from the group consisting of substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl;
R^{24b} is hydrogen, substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, or substituted or unsubstituted 5- to 10-membered heteroaryl;
or R^{24a} and R^{24b}, taken together with the carbon atom to which they are attached, form a substituted or unsubstituted C₃₋₆carbocyclyl or a substituted or unsubstituted 3- to 10-membered heterocyclyl;
q is 0, 1, or 2;
r is 1 or 2;
s is 1, 2, or 3; and
each [symbol] independently represents a single or double bond, provided if the bond between C5 and C6 is a double bond, R⁵ is absent and if the bond between C9 and C11 is a double bond, R⁹ and R^{11b} are absent.

2. The compound or pharmaceutically acceptable salt thereof according to claim 1, wherein the compound of Formula (A-I) is selected from the group consisting of:

| Cmpd | Structure |
|---|---|
| A-1 | 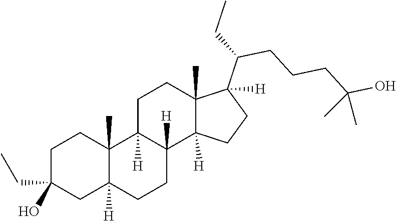 |
| A-2 | 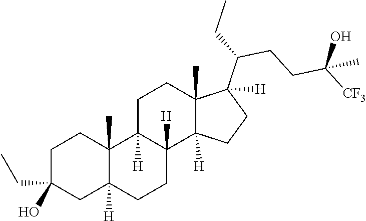 |
| A-3 | 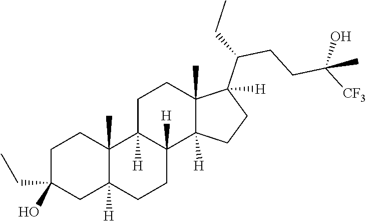 |
| A-4 | 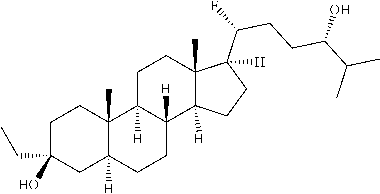 |
| A-5 | 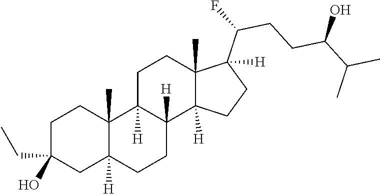 |
| A-6 | 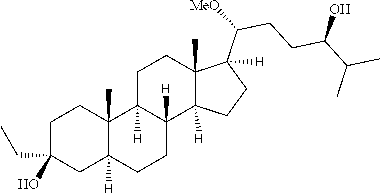 |
| A-7 | 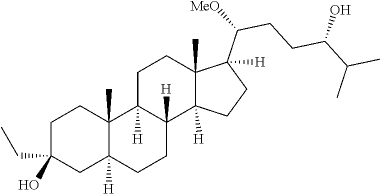 |
| A-8 | 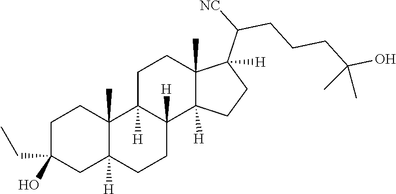 |
| A-9 | 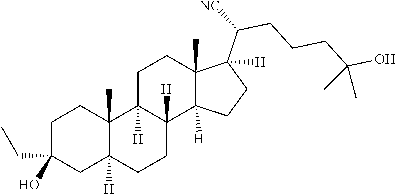 |
| A-9a | 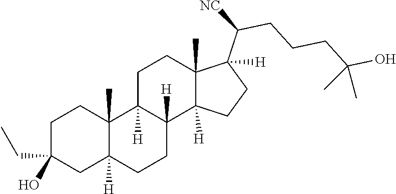 |
| A-10 | 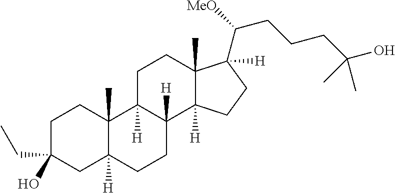 |
| A-11 | 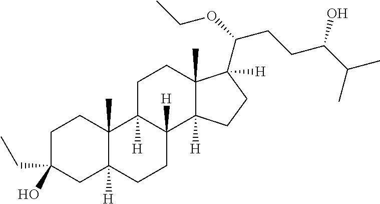 |
| A-11a | 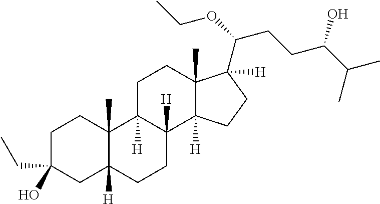 |
| A-12 | 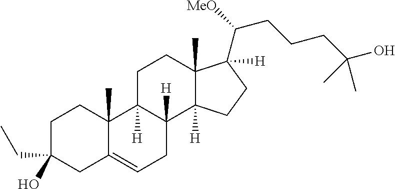 |
| A-13 | 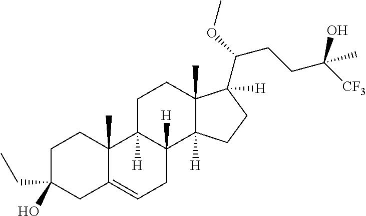 |
| A-14 | 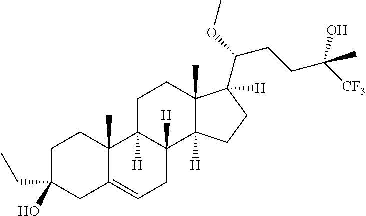 |
| A-15 | 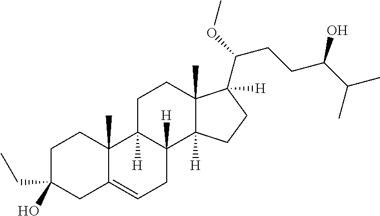 |
| A-16 | 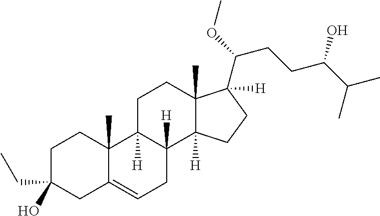 |
| A-17 | 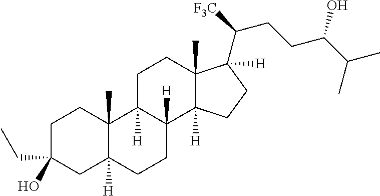 |
| A-18 | 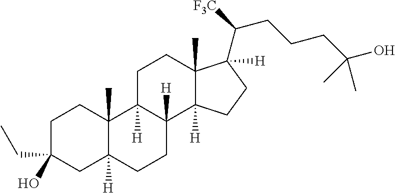 |
| A-19 | 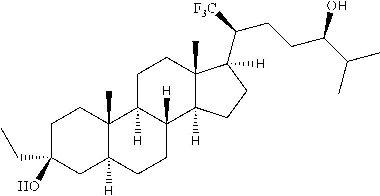 |
| A-20 | 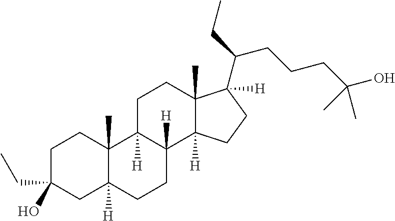 |
| A-21 | 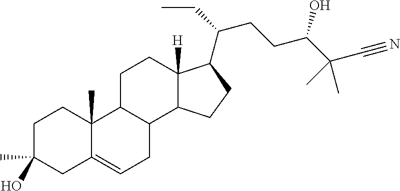 |
| A-22 | 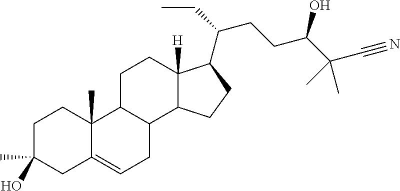 |
| A-23 | 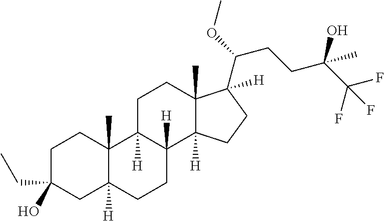 |
| A-24 | 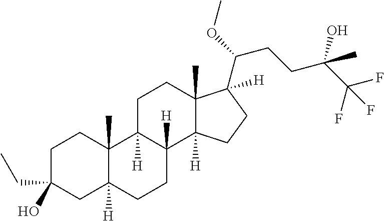 |
| A-25 | 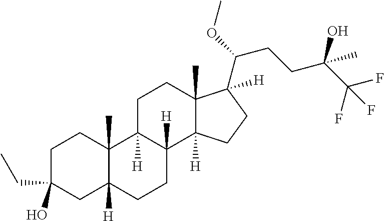 |
| A-26 | 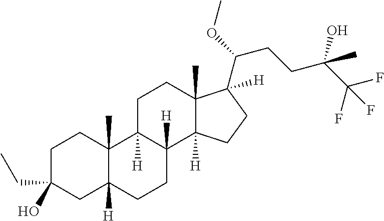 |

and pharmaceutically acceptable salts thereof.

3. A compound of Formula (B-I): 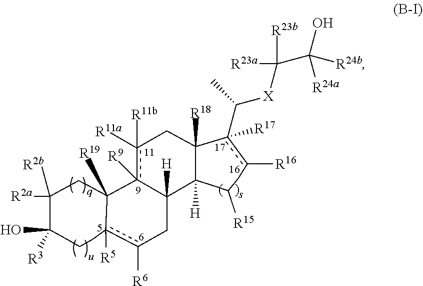
or a pharmaceutically acceptable salt thereof;
wherein:
each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein R^{A2} is hydrogen or substituted or unsubstituted C₁₋₆alkyl;
R³ is substituted or unsubstituted C₁₋₆alkyl, substituted or unsubstituted C₂₋₆alkenyl, substituted or unsubstituted C₂₋₆alkynyl, or substituted or unsubstituted C₃₋₁₀carbocyclyl;
R⁵ is absent, hydrogen, halogen, or substituted or unsubstituted C₁₋₆alkyl;
R⁶ is hydrogen, substituted or unsubstituted C₁₋₆alkyl, or halogen;
R⁹ is absent or hydrogen; or
R⁹ and R^{11a} are taken together with the carbon atoms to which they are attached to form a substituted or unsubstituted C₃₋₆carbocyclyl;
R¹¹ª is hydrogen, halogen, substituted or unsubstituted C₁₋₆alkyl, or —OR^{A11}, wherein R^{A11} is hydrogen or substituted or unsubstituted C₁₋₆alkyl;
R^{11b} is absent or hydrogen; or
R^{11a} and R^{11b} are joined to form an oxo (═O) group;
R¹⁵ is hydrogen, halogen, —OH, —NH₂, substituted or unsubstituted C₁₋₆alkyl, substituted or unsubstituted C₂₋₆alkenyl, substituted or unsubstituted C₂₋₆alkynyl, substituted or unsubstituted C₁₋₆alkoxy, substituted or unsubstituted C₃₋₆carbocyclyl or substituted or unsubstituted 3- to 10-membered heterocyclyl;
R¹⁶ is hydrogen, halogen —OH, —NH₂, substituted or unsubstituted C₁₋₆alkyl, substituted or unsubstituted C₂₋₆alkenyl, substituted or unsubstituted C₂₋₆alkynyl, substituted or unsubstituted C₁₋₆alkoxy, substituted or unsubstituted C₃₋₆carbocyclyl or substituted or unsubstituted 3- to 10-membered heterocyclyl;
R¹⁷ is absent, hydrogen or substituted or unsubstituted C₁₋₆alkyl; provided that when the bond between C16 and C17 is a double bond, then R¹⁷ is absent;
or R¹⁵ and R¹⁶ are taken together with the carbon atoms to which they are attached, to form a substituted or unsubstituted C₃₋₆carbocyclyl;
or R¹⁶ and R¹⁷ are taken together with the carbon atoms to which they are attached to form a substituted or unsubstituted C₃₋₆carbocyclyl;
provided that R¹⁵ and R¹⁶ and R¹⁶ and R¹⁷ cannot both be taken together with the carbon atoms to which they are attached, to form a substituted or unsubstituted C₃₋₆carbocyclyl; and
provided that at least one of R¹⁵, R¹⁶, and R¹⁷ is not hydrogen;
R¹⁸ is hydrogen, substituted or unsubstituted C₁₋₆alkyl, OR^{A18}, wherein R^{A18} is substituted or unsubstituted C₁₋₆alkyl;
R¹⁹ is hydrogen, substituted or unsubstituted C₁₋₆alkyl, OR^{A19}, wherein R^{A19} is substituted or unsubstituted C₁₋₆alkyl;
X is —(C(R^{X})₂)ₙ— or —O—, wherein R^{X} is hydrogen or fluorine, or one R^{X} group and R^{23b} are joined to form a double bond;
n is 1, 2 or 3;
each instance of R^{23a} and R^{23b} is independently hydrogen or fluorine;
R^{24a} is selected from the group consisting of substituted or unsubstituted C₁₋₆alkyl, substituted or unsubstituted C₂₋₆alkenyl, substituted or unsubstituted C₂₋₆ alkynyl, substituted or unsubstituted C₃₋₁₀carbocyclyl, substituted or unsubstituted 3- to 10-membered heterocyclyl, substituted or unsubstituted C₆₋₁₄ aryl, and substituted or unsubstituted 5- to 10-membered heteroaryl;
R^{24b} is hydrogen or substituted or unsubstituted C₁₋₆alkyl;
or R^{24a} and R^{24b}, taken together with the carbon atom to which they are attached, form a substituted or unsubstituted C₃₋₆carbocyclyl, or substituted or unsubstituted 3- to 10-membered heterocyclyl;
q is 0, 1 or 2;
u is 1 or 2;
s is 1 or 2; and
each [symbol] independently represents a single or double bond, provided if the bond between C5 and C6 is a double bond, R⁵ is absent, and if the bond between C9 and C11 is a double bond, R⁹ and R^{11b} are absent.

4. The compound or pharmaceutically acceptable salt thereof according to claim 3, wherein the compound of formula (B-I) is selected from the group consisting of:

| Cmpd | Structure |
|---|---|
| B-1 | 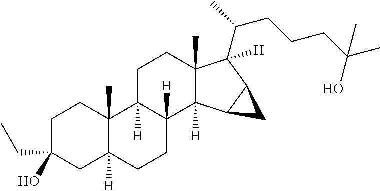 |
| B-2 | 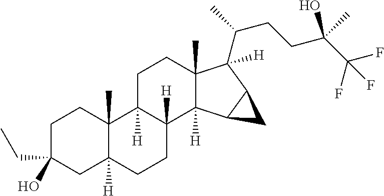 |
| B-3 | 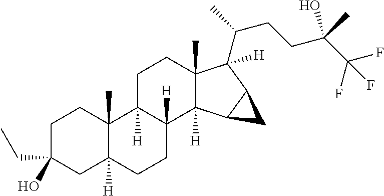 |
| B-4 | 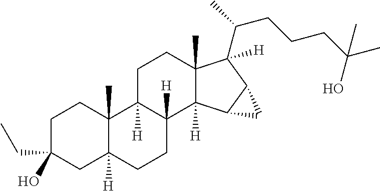 |
| B-5 | 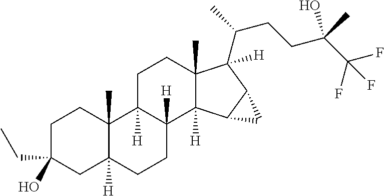 |
| B-6 | 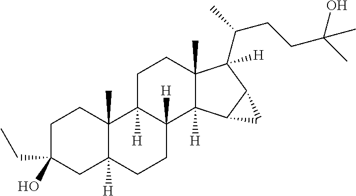 |
| B-7 | 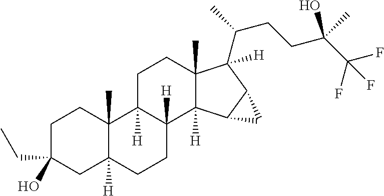 |
| B-8 | 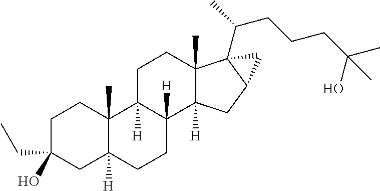 |
| B-10 | 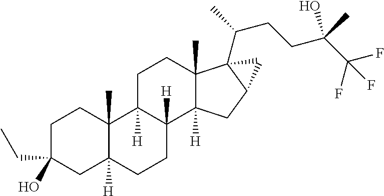 |
| B-11 | 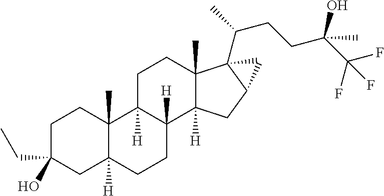 |
| B-12 | 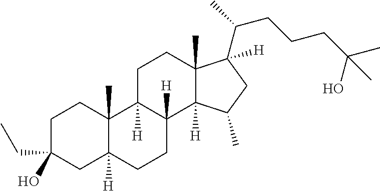 |
| B-13 | 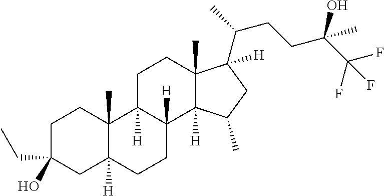 |
| B-14 | 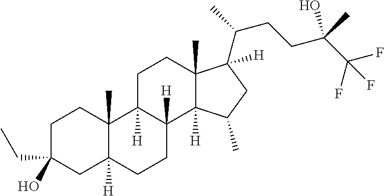 |
| B-15 | 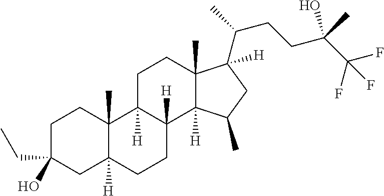 |
| B-16 | 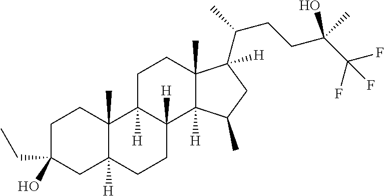 |
| B-17 | 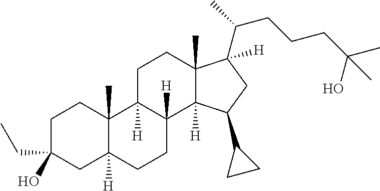 |
| B-18 | 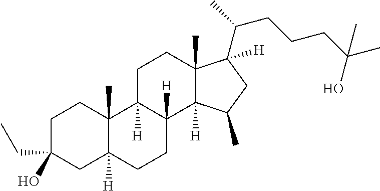 |
| B-19 | 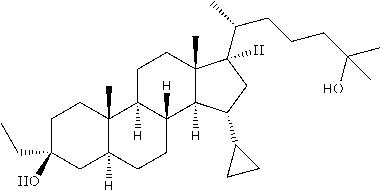 |
| B-20 | 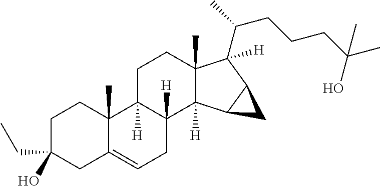 |
| B-21 | 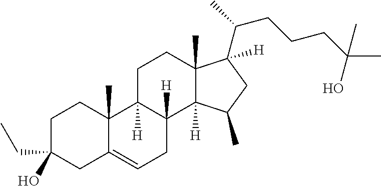 |
| B-22 | 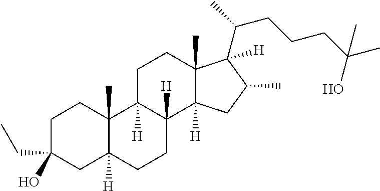 |
| B-23 | 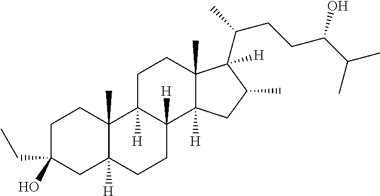 |
| B-24 | 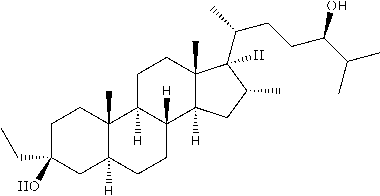 |

and pharmaceutically acceptable salts thereof.

5. A compound of Formula (C-I): 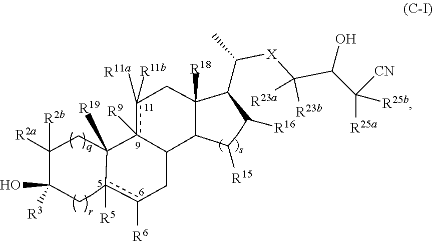
or a pharmaceutically acceptable salt thereof;
wherein:
each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein R^{A2} is hydrogen or substituted or unsubstituted C₁₋₆alkyl;
R³ is substituted or unsubstituted C₁₋₆alkyl, substituted or unsubstituted C₂₋₆alkenyl, or substituted or unsubstituted C₂₋₆alkynyl, or substituted or unsubstituted C₃₋₁₀carbocyclyl;
R⁵ is absent, hydrogen, halogen, or substituted or unsubstituted C₁₋₆alkyl;
R⁶ is hydrogen, halogen, or substituted or unsubstituted C₁₋₆alkyl;
R⁹ is absent or hydrogen; or
R⁹ and R^{11a} are taken together with the carbon atoms to which they are attached to form a substituted or unsubstituted C₃₋₆carbocyclyl ring;
R^{11a} is hydrogen, halogen, substituted or unsubstituted C₁₋₆alkyl, or —OR^{A11}, wherein R^{A11} is hydrogen or substituted or unsubstituted C₁₋₆alkyl;
R^{11b} is absent or hydrogen; or
R^{11a} and R^{11b} are joined to form an oxo (═O) group;
each instance of R¹⁵ is hydrogen, halogen, —OH, —NH₂, substituted or unsubstituted C₁₋₆ alkyl, substituted or unsubstituted C₂₋₆alkenyl, substituted or unsubstituted C₂₋₆alkynyl, substituted or unsubstituted C₁₋₆alkoxy, substituted or unsubstituted C₃₋₆carbocyclyl, or substituted or unsubstituted 3- to 10-membered heterocyclyl;
R¹⁶ is hydrogen, halogen, —OH, —NH₂, substituted or unsubstituted C₁₋₆alkyl, substituted or unsubstituted C₂₋₆alkenyl, substituted or unsubstituted C₂₋₆alkynyl, substituted or unsubstituted C₁₋₆alkoxy, substituted or unsubstituted C₃₋₆carbocyclyl, or substituted or unsubstituted 3- to 10-membered heterocyclyl; or
R¹⁵ and R¹⁶ are taken together with the carbon atoms to which they are attached, to form a substituted or unsubstituted C₃₋₆carbocyclyl;
R¹⁸ is hydrogen, substituted or unsubstituted C₁₋₆alkyl, or OR^{A18}, wherein each instance of R^{A18} is independently substituted or unsubstituted C₁₋₆alkyl;
R¹⁹ is hydrogen, substituted or unsubstituted C₁₋₆alkyl, or OR^{A19}; wherein each instance of R^{A19} is independently substituted or unsubstituted C₁₋₆alkyl;
X is —(C(R^{X})₂)ₙ— or —O—, wherein R^{X} is hydrogen or fluorine, or one R^{X} group and R^{23b} are joined to form a double bond;
n is 1, 2 or 3;
each of R^{23a} and R^{23b} is independently hydrogen or fluorine;
R^{25a} is hydrogen, substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, or substituted or unsubstituted 5- to 10-membered heteroaryl;
R^{25b} is hydrogen, substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, or substituted or unsubstituted 5- to 10-membered heteroaryl;
or R^{25a} and R^{25b}, taken together with the carbon atom to which they are attached, form a substituted or unsubstituted C₃₋₆carbocyclyl or substituted or unsubstituted 3- to 10-membered heterocyclyl;
q is 0, 1 or 2;
r is 1 or 2;
s is 1 or 2; and
each [symbol] independently represents a single or double bond;
provided if the bond between C5 and C6 is a double bond, R⁵ is absent and if the bond between C9 and C11 is a double bond, R⁹ and R^{11b} are absent.

6. The compound or pharmaceutically acceptable salt thereof according to claim 5, wherein the compound of Formula (C-I) is selected from the group consisting of:

| Cmpd | Structure |
|---|---|
| C-1 | 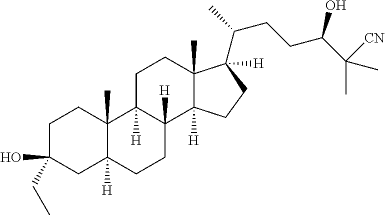 |
| C-2 | 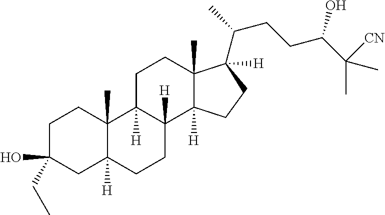 |
| C-3 | 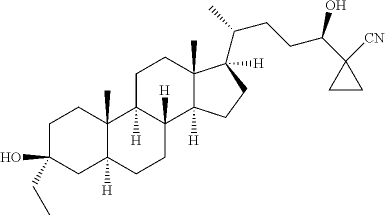 |
| C-4 | 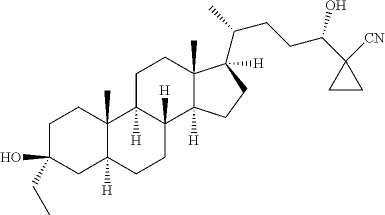 |
| C-5 | 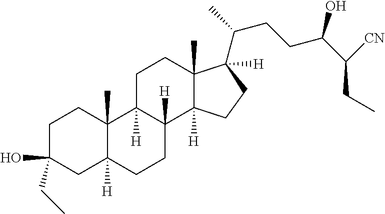 |
| C-6 | 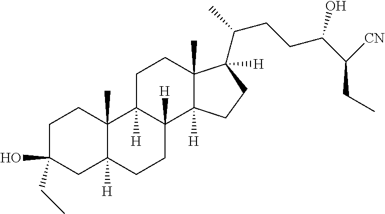 |
| C-7 | 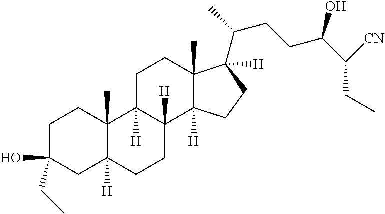 |
| C-8 | 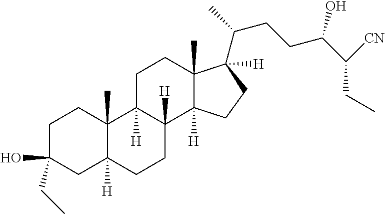 |
| C-9 | 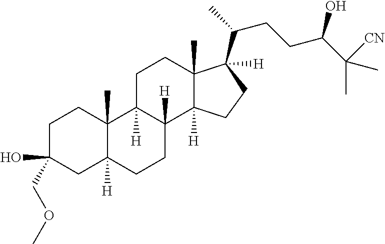 |
| C-10 | 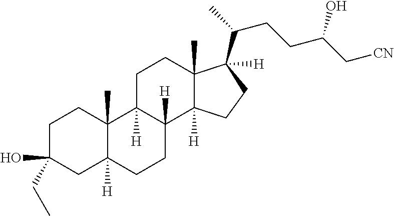 |
| C-11 | 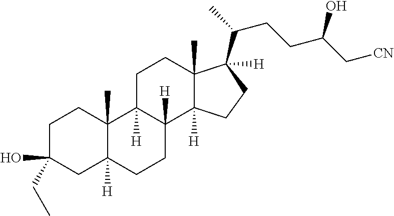 |
| C-12 | 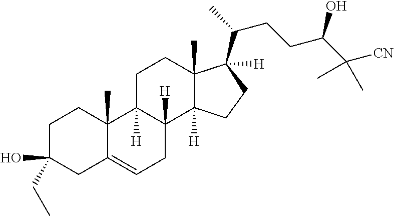 |
| C-13 | 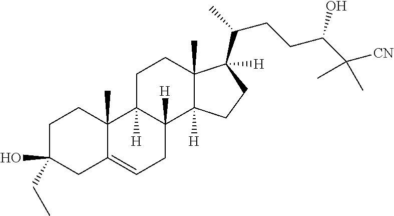 |
| C-14 | 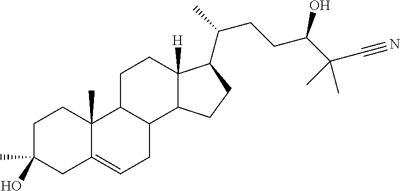 |
| C-15 | 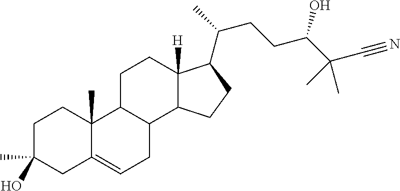 |
| C-16 | 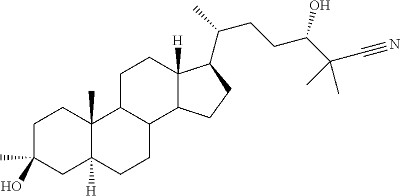 |
| C-17 | 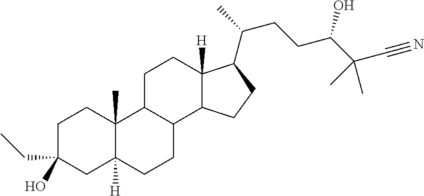 |
| C-18 | 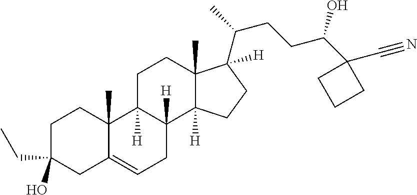 |
| C-19 | 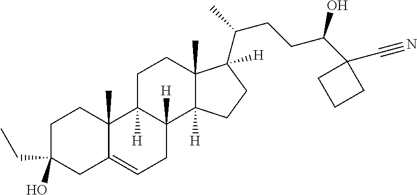 |
| C-20 | 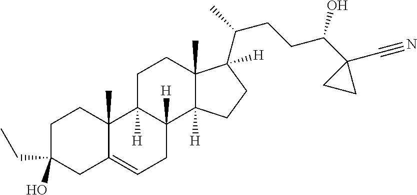 |
| C-21 | 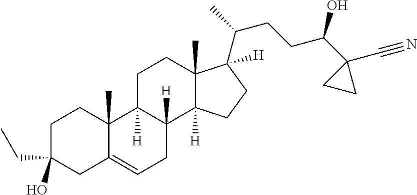 |
| C-22 | 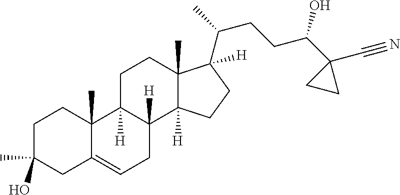 |
| C-23 | 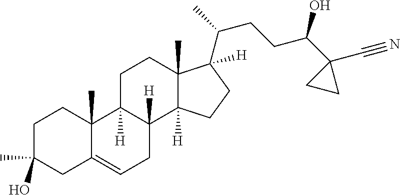 |
| C-24 | 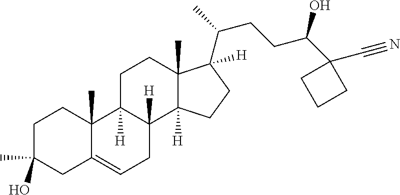 |
| C-25 | 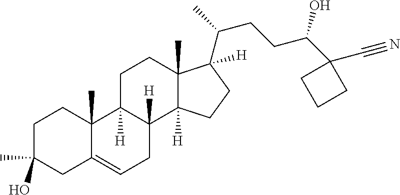 |
| C-26 | 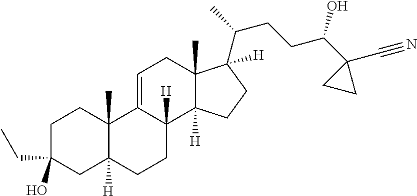 |
| C-27 | 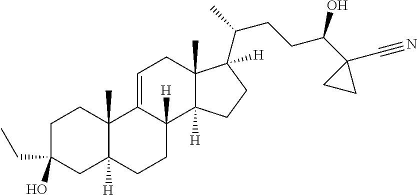 |
| C-28 | 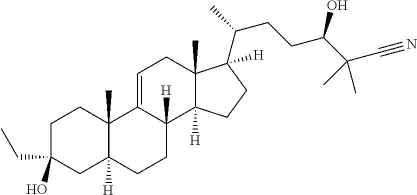 |
| C-29 | 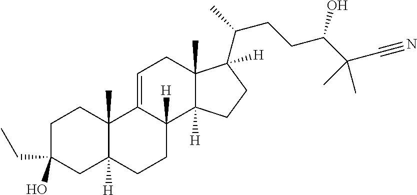 |
| C-30 | 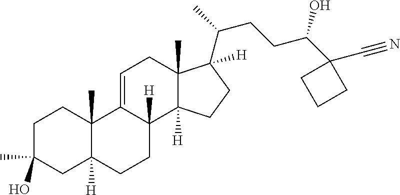 |
| C-31 | 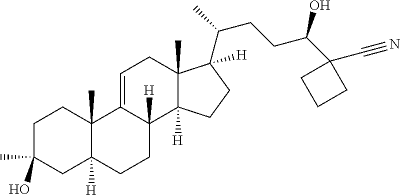 |
| C-32 | 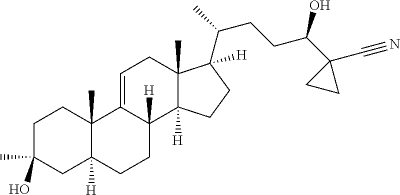 |
| C-33 | 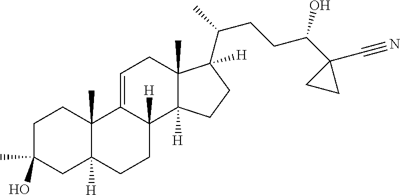 |
| C-34 | 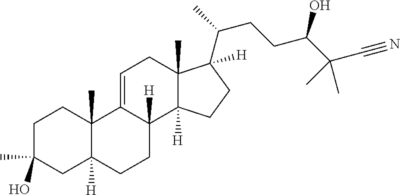 |
| C-35 | 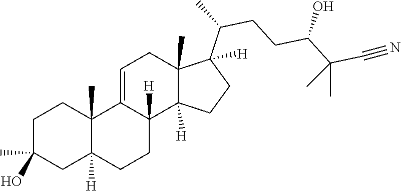 |

and pharmaceutically acceptable salts thereof.

7. A compound of Formula (D-I): 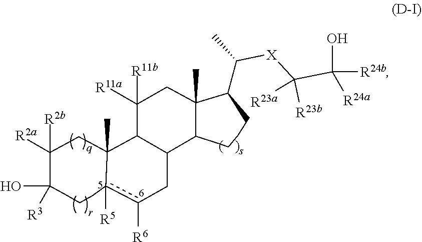
or a pharmaceutically acceptable salt thereof;
wherein:
each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein R^{A2} is hydrogen or substituted or unsubstituted C₁₋₆alkyl;
R³ is substituted or unsubstituted C₁₋₆alkyl, substituted or unsubstituted C₂₋₆ alkenyl, or substituted or unsubstituted C₂₋₆alkynyl, or substituted or unsubstituted C₃₋₁₀ carbocyclyl;
R⁵ is absent, hydrogen, halogen, or substituted or unsubstituted C₁₋₆alkyl;
R⁶ is hydrogen, substituted or unsubstituted C₁₋₆alkyl, or halogen;
R^{11a} is hydrogen or —OR^{A11}, wherein R^{A11} is hydrogen or substituted or unsubstituted C₁₋₆alkyl, and R^{11b} is hydrogen; or R^{11a} and R^{11b} are joined to form an oxo (═O) group;
X is —(C(R^{X})₂)ₙ— or —O—, wherein R^{X} is hydrogen or fluorine, or one R^{X} group and R^{23b} are joined to form a double bond;
n is 1, 2 or 3;
each of R^{23a} and R^{23b} is independently hydrogen or fluorine;
R^{24a} is selected from the group consisting of substituted or unsubstituted C₁₋₆alkyl, substituted or unsubstituted C₂₋₆alkenyl, substituted or unsubstituted C₂₋₆ alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl group; and
R^{24b} is hydrogen or a substituted or unsubstituted C₁₋₆alkyl;
or R^{24a} and R^{24b}, taken together with the carbon atom to which they are attached, form a substituted or unsubstituted C₃₋₆carbocyclyl, or substituted or unsubstituted 3- to 10-membered heterocyclyl;
q is 0, 1 or 2;
r is 1 or 2;
s is 1, 2, 3 or 4;
provided that a) q and r are not both 1, or b) when q is 0, r is not 2; and [symbol]
represents a single or double bond; provided if the bond between C5 and C6 is a double bond, R⁵ is absent.

8. The compound or pharmaceutically acceptable salt thereof according to claim 7, wherein the compound of Formula (D-I) is selected from the group consisting of:

| Com- | |
|---|---|
| pound | Structure |
| D-1 | 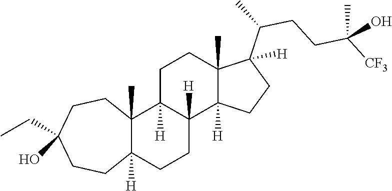 |
| D-2 | 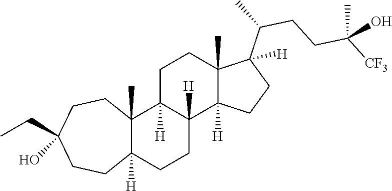 |
| D-3 | 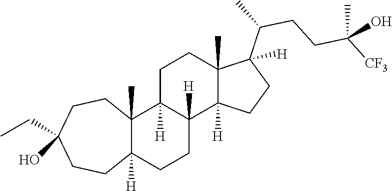 |
| D-4 | 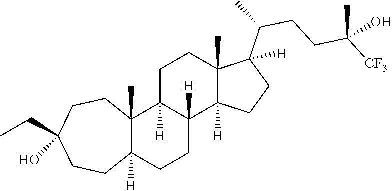 |
| D-5 | 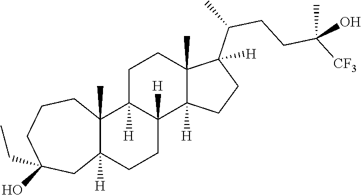 |
| D-6 | 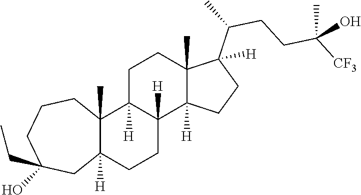 |
| D-7 | 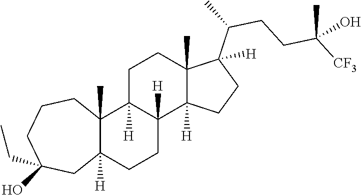 |
| D-8 | 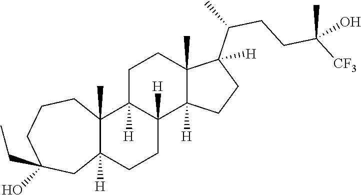 |
| D-9 | 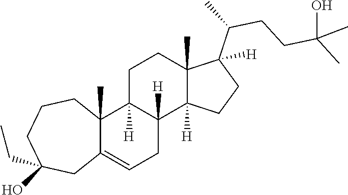 |
| D-9a | 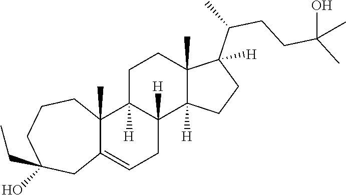 |
| D-10 | 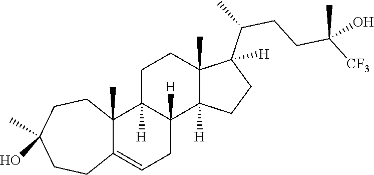 |
| D-11 | 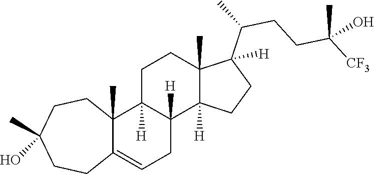 |
| D-12 | 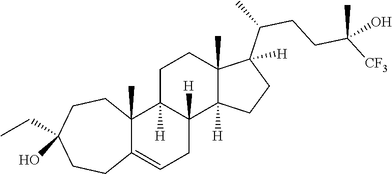 |
| D-13 | 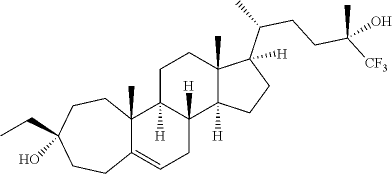 |
| D-14 | 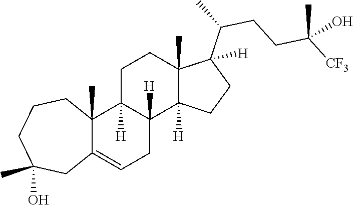 |
| D-15 | 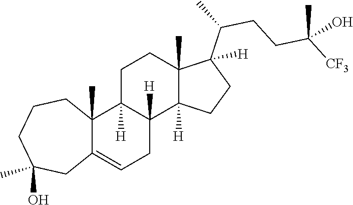 |
| D-16 | 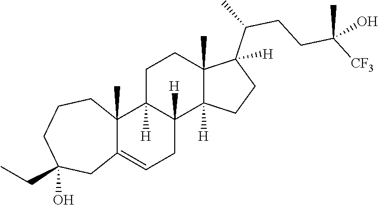 |
| D-17 | 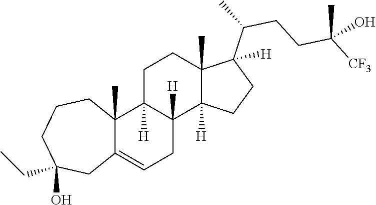 |

and pharmaceutically acceptable salts thereof.

9. A compound of Formula (E-I): 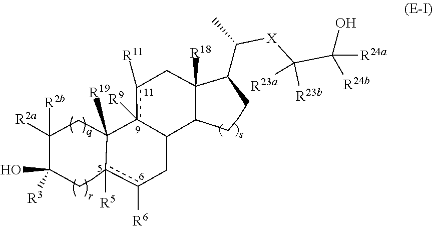
or a pharmaceutically acceptable salt thereof;
wherein:
each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein R^{A2} is hydrogen or substituted or unsubstituted C₁₋₆alkyl;
R³ is substituted or unsubstituted C₁₋₆alkyl, substituted or unsubstituted C₂₋₆ alkenyl, or substituted or unsubstituted C₂₋₆alkynyl, or substituted or unsubstituted C₃₋₁₀ carbocyclyl; [symbol]
represents a single or double bond;
when the bond between C9 and C11 is a double bond, R⁹ is absent and R¹¹ is selected from the group consisting of hydrogen, halogen, substituted or unsubstituted C₁₋₆alkyl, substituted or unsubstituted C₂₋₆alkenyl, substituted or unsubstituted C₂₋₆alkynyl, substituted or unsubstituted C₃₋₁₀carbocyclyl, substituted or unsubstituted C₁₋₆alkoxy, substituted or unsubstituted 3- to 10-membered heterocyclyl, —OH, and amino;
when the bond between C9 and C11 is a single bond, then R⁹ is hydrogen and R¹¹ is halogen or substituted or unsubstituted C₁₋₆alkyl; or R⁹ and R¹¹, taken together with the carbon atoms to which they are attached, form a substituted or unsubstituted C₃₋₆carbocyclyl ring;
when the bond between C5 and C6 is a double bond, then R⁵ is absent and R⁶ is hydrogen, halogen, or substituted or unsubstituted C₁₋₆alkyl;
when the bond between C5 and C6 is a single bond, then each of R⁵ and R⁶ is independently hydrogen, halogen, or substituted or unsubstituted C₁₋₆alkyl;
R¹⁸ is hydrogen, substituted or unsubstituted C₁₋₆alkyl, or OR^{A18}, wherein R^{A18} is substituted or unsubstituted C₁₋₆alkyl;
R¹⁹ is hydrogen, substituted or unsubstituted C₁₋₆alkyl, or OR^{A19}, wherein R^{A19} is substituted or unsubstituted C₁₋₆alkyl;
X is —(C(R^{X})₂)ₙ— or —O—, wherein R^{X} is hydrogen or fluorine, or one R^{X} group and R^{23b} are joined to form a double bond;
n is 1 or 2;
each of R^{23a} and R^{23b} is independently hydrogen or fluorine;
R^{24a} is selected from the group consisting of substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl;
R^{24b} is hydrogen or a substituted or unsubstituted C₁₋₆alkyl; or
R^{24a} and R^{24b}, taken together with the carbon atoms to which they are attached, form a substituted or unsubstituted C₃₋₆carbocyclyl, or substituted or unsubstituted 3- to 6-membered heterocyclyl;
q is 0, 1 or 2;
r is 1 or 2; and
s is 1 or 2.

10. The compound or pharmaceutically acceptable salt thereof according to claim 9, wherein the compound of Formula (E-I) is selected from the group consisting of:

| Cmpd | Structure |
|---|---|
| E-1 | 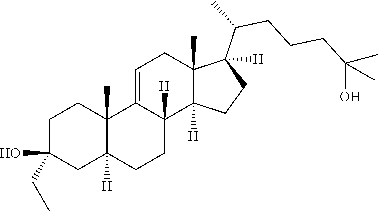 |
| E-2 | 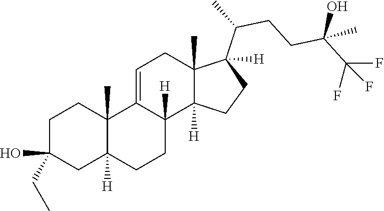 |
| E-3 | 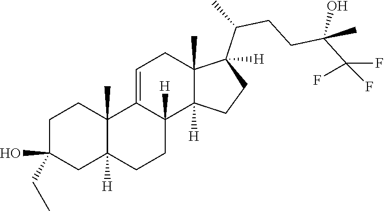 |
| E-4 | 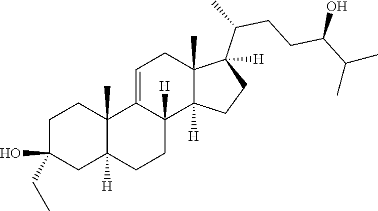 |
| E-5 | 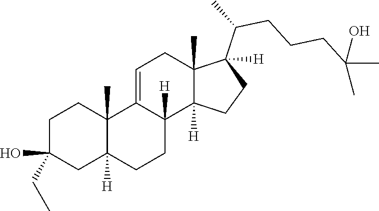 |
| E-6 | 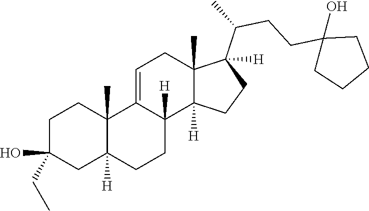 |
| E-7 | 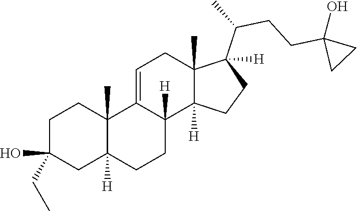 |
| E-8 | 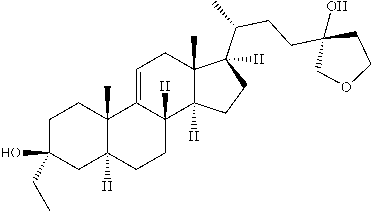 |
| E-9 | 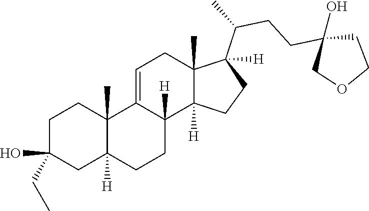 |
| E-10 | 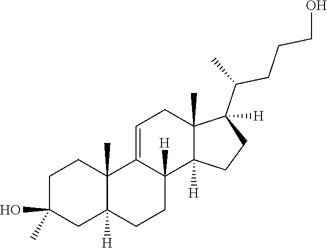 |
| E-11 | 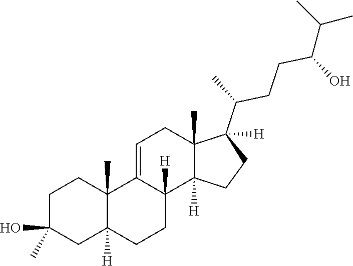 |
| E-12 | 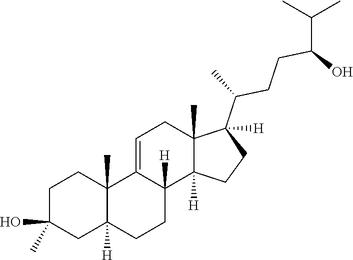 |
| E-13 | 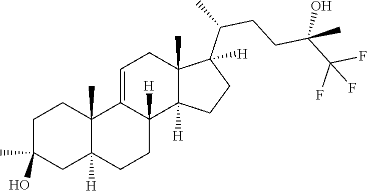 |
| E-14 | 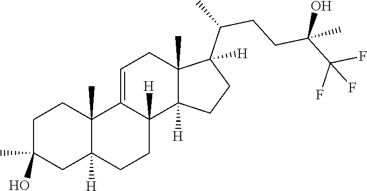 |
| E-15 | 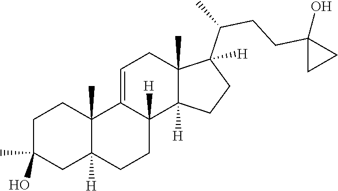 |
| E-16 | 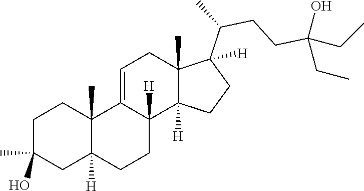 |
| C-26 | 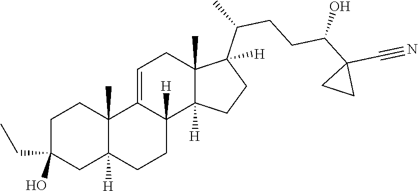 |
| C-27 | 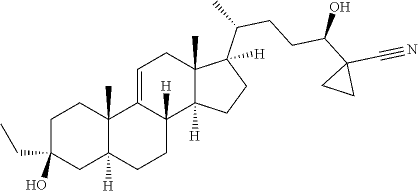 |
| C-28 | 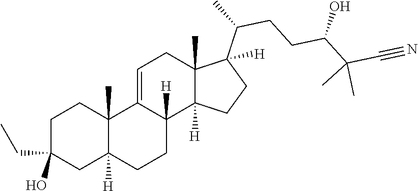 |
| C-29 | 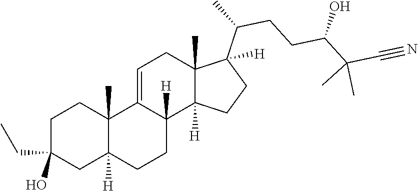 |
| C-30 | 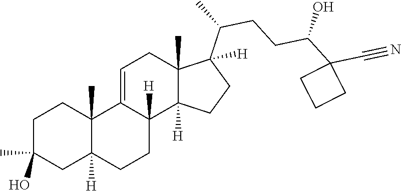 |
| C-31 | 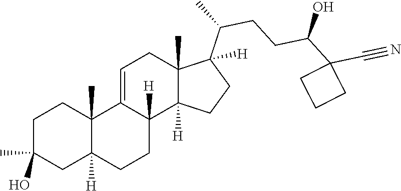 |
| C-32 | 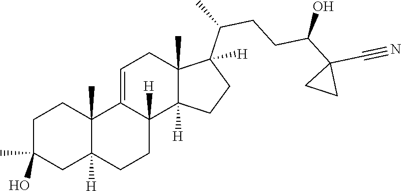 |
| C-33 | 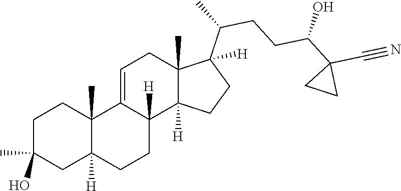 |
| C-34 | 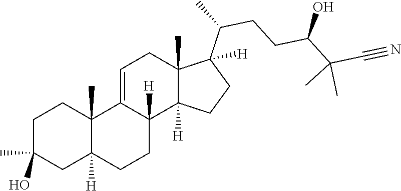 |
| C-35 | 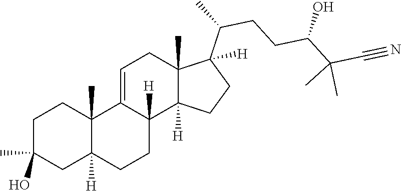 |

and pharmaceutically acceptable salts thereof.

11. A compound of Formula (F-I): 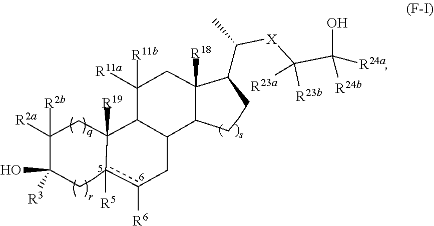
or a pharmaceutically acceptable salt thereof;
wherein:
each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein R^{A2} is hydrogen or substituted or unsubstituted C₁₋₆alkyl;
R³ is substituted or unsubstituted C₁₋₆alkyl, substituted or unsubstituted C₂₋₆ alkenyl, substituted or unsubstituted C₂₋₆alkynyl, or substituted or unsubstituted C₃₋₁₀carbocyclyl;
R⁵ is absent, hydrogen, substituted or unsubstituted C₁₋₆alkyl, or halogen;
R⁶ is hydrogen, substituted or unsubstituted C₁₋₆alkyl, or halogen;
R^{11a} is hydrogen or —OR^{A11}, wherein R^{A11} is hydrogen or substituted or unsubstituted C₁₋₆alkyl, and R^{11b} is hydrogen; or R^{11a} and R^{11b} are joined to form an oxo (═O) group;
R¹⁸ is hydrogen, unsubstituted C₂₋₆alkyl, substituted C₁₋₆alkyl, or OR^{A18}, wherein each instance of R^{A18} is independently substituted or unsubstituted C₁₋₆alkyl;
R¹⁹ is hydrogen, substituted or unsubstituted C₁₋₆alkyl, or OR^{A19}, each instance of R^{A19} is independently substituted or unsubstituted C₁₋₆alkyl;
X is —(C(R^{X})₂)ₙ— or —O—, wherein R^{X} is hydrogen or fluorine, or one R^{X} group and R^{23b} are joined to form a double bond;
n is 1, 2 or 3;
each of R^{23a} and R^{23b} is independently hydrogen or fluorine;
R^{24a} is selected from the group consisting of substituted or unsubstituted C₁₋₆ alkyl, substituted or unsubstituted C₂₋₆alkenyl, substituted or unsubstituted C₂₋₆ alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl; and
R^{24b} is hydrogen or substituted or unsubstituted C₁₋₆alkyl;
or R^{24a} and R^{24b}, taken together with the carbon atom to which they are attached, form a substituted or unsubstituted C₃₋₆carbocyclyl, or a substituted or unsubstituted 3- to 10-membered heterocyclyl;
q is 0, 1 or 2;
r is 1 or 2;
s is 1 or 2; and [symbol]
represents a single or double bond, provided if a double bond is present between C5 and C6, R⁵ is absent.

12. The compound or pharmaceutically acceptable salt thereof according to claim 11, wherein the compound of formula (F-I) is selected from the group consisting of:

| Cmpd | Structure |
|---|---|
| F-1 | 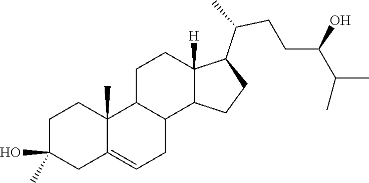 |
| F-2 | 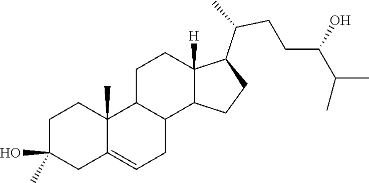 |
| F-3 | 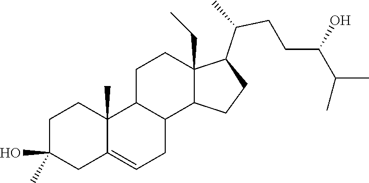 |
| F-4 | 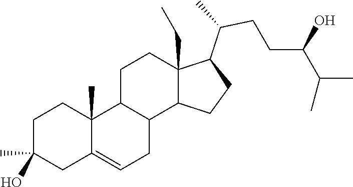 |
| F-5 | 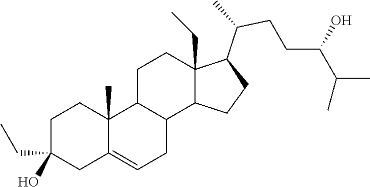 |
| F-6 | 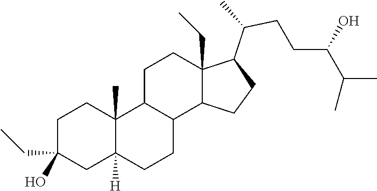 |
| F-7 | 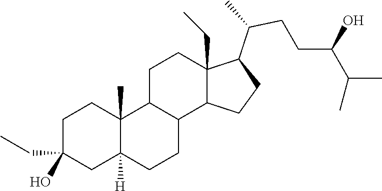 |
| F-8 | 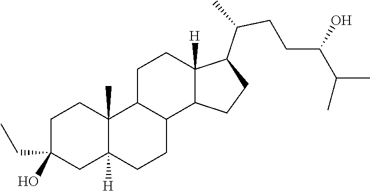 |
| F-9 | 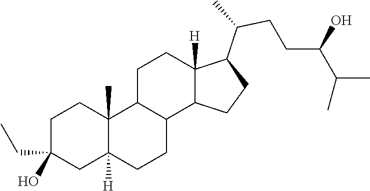 |
| F-10 | 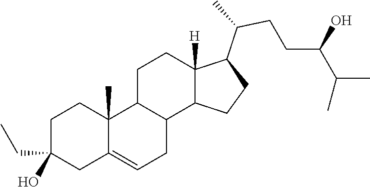 |
| F-11 | 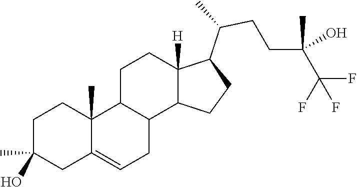 |
| F-12 | 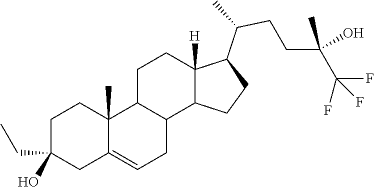 |
| F-13 | 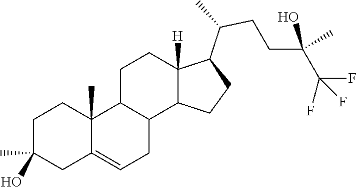 |
| F-14 | 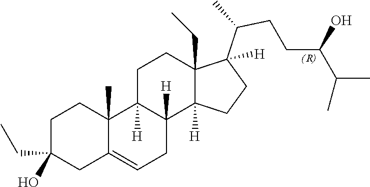 |

and pharmaceutically acceptable salts thereof.

13. A compound of Formula (G-I): 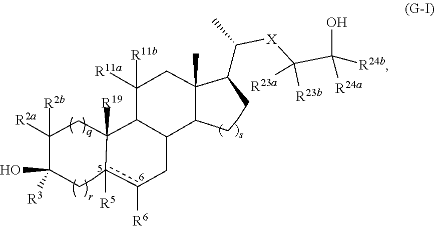
or a pharmaceutically acceptable salt thereof;
wherein:
each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein R^{A2} is hydrogen or substituted or unsubstituted C₁₋₆alkyl;
R³ is substituted or unsubstituted C₁₋₆alkyl, substituted or unsubstituted C₂₋₆ alkenyl, or substituted or unsubstituted C₂₋₆alkynyl;
R⁵ is absent, hydrogen, substituted or unsubstituted C₁₋₆alkyl, or halogen;
R⁶ is hydrogen, substituted or unsubstituted C₁₋₆alkyl, or halogen;
R¹⁹ is hydrogen, substituted or unsubstituted C₂₋₆alkyl, or OR^{A19}; wherein each instance of R^{A19} is independently substituted or unsubstituted C₁₋₆alkyl;
R^{11a} is hydrogen or —OR^{A11}, wherein R^{A11} is hydrogen or substituted or unsubstituted C₁₋₆alkyl, and R^{11b} is hydrogen; or R^{11a} and R^{11b} are joined to form an oxo (═O) group;
X is —(C(R^{X})₂)ₙ— or —O—, wherein R^{X} is hydrogen or fluorine, or one R^{X} group and R^{23b} are joined to form a double bond;
n is 1, 2 or 3;
each of R^{23a} and R^{23b} is independently hydrogen or fluorine;
R^{24a} is selected from the group consisting of substituted or unsubstituted C₁₋₆alkyl, substituted or unsubstituted C₂₋₆alkenyl, substituted or unsubstituted C₂₋₆alkynyl, substituted or unsubstituted C₃₋₆carbocyclyl, substituted or unsubstituted 3- to 10-membered heterocyclyl, substituted or unsubstituted C₆₋₁₄ aryl, and substituted or unsubstituted 5- to 10-membered heteroaryl; and
R^{24b} is hydrogen or substituted or unsubstituted C₁₋₆alkyl;
or R^{24a} and R^{24b}, taken together with the carbon atom to which they are attached, form a substituted or unsubstituted C₃₋₆carbocyclyl;
q is 0, 1 or 2;
r is 1 or 2;
s is 1, 2, 3 or 4; and [symbol]
represents a single or double bond; provided if the bond between C5 and C6 is a double bond, R⁵ is absent.

14. The compound or pharmaceutically acceptable salt thereof according to claim 13, wherein the compound of Formula (G-I) is selected from the group consisting of:

| Cmpd | Structure |
|---|---|
| G-1 | 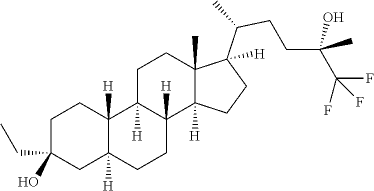 |
| G-2 | 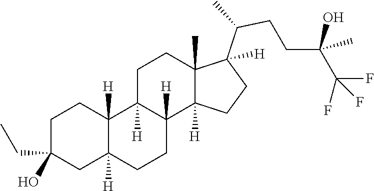 |
| G-3 | 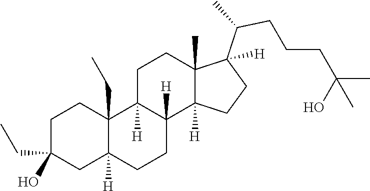 |
| G-4 | 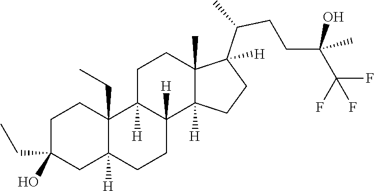 |
| | and |
| G-5 | 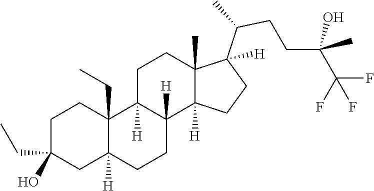 |

and pharmaceutically acceptable salts thereof.

15. A compound of Formula (H-I): 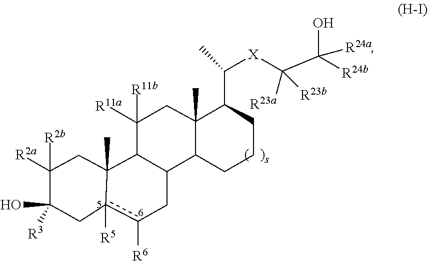
or a pharmaceutically acceptable salt thereof;
wherein:
each of R^{2a} and R^{2b} is independently hydrogen, halogen, substituted or unsubstituted C₁₋₆alkyl, substituted or unsubstituted C₃₋₁₀carbocyclyl, or —OR^{A2}, wherein R^{A2} is hydrogen or substituted or unsubstituted C₁₋₆alkyl;
R³ is substituted or unsubstituted C₁₋₆alkyl, substituted or unsubstituted C₂₋₆alkenyl, substituted or unsubstituted C₂₋₆alkynyl, or substituted or unsubstituted C₃₋₁₀carbocyclyl;
R⁵ is absent, hydrogen, halogen, or substituted or unsubstituted C₁₋₆alkyl
R⁶ is hydrogen, substituted or unsubstituted C₁₋₆alkyl, or halogen;
R^{11a} is hydrogen or —OR^{A11}, wherein R^{A11} is hydrogen or substituted or unsubstituted alkyl, and R^{11b} is hydrogen; or R^{11a} and R^{11b} are joined to form an oxo (═O) group;
X is —(C(R^{X})₂)ₙ— or —O—, wherein R^{X} is hydrogen or fluorine, or one R^{X} group and R^{23b} are joined to form a double bond;
n is 1, 2 or 3;
each of R^{23a} and R^{23b} is independently hydrogen or fluorine;
R^{24a} is selected from the group consisting of substituted or unsubstituted alkyl, substituted or unsubstituted alkenyl, substituted or unsubstituted alkynyl, substituted or unsubstituted carbocyclyl, substituted or unsubstituted heterocyclyl, substituted or unsubstituted aryl, and substituted or unsubstituted heteroaryl group; and
R^{24b} is hydrogen or substituted or unsubstituted alkyl;
or R^{24a} and R^{24b}, taken together with the carbon atom to which they are attached, form a substituted or unsubstituted C₃₋₆carbocyclyl or substituted or unsubstituted 3- to 10-membered heterocyclyl;
s is 1, 2 or 3; and [symbol]
represents a single or double bond, provided if the bond between C5 and C6 is a double bond, R⁵ is absent.

16. The compound or pharmaceutically acceptable salt thereof according to claim 15, wherein the compound of Formula (H-I) is selected from the group consisting of:

| Com- | |
|---|---|
| pound | Structure |
| H-1 | 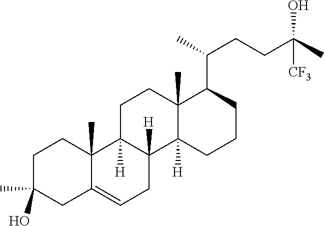 |
| H-2 | 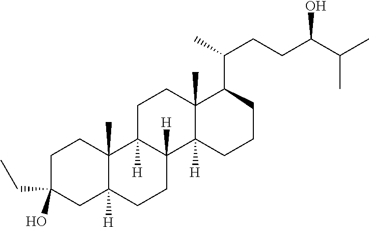 |
| H-3 | 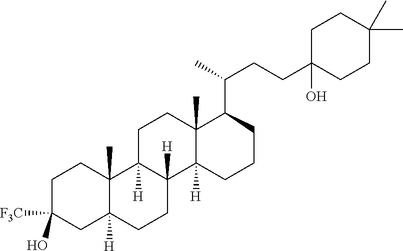 |
| H-4 | 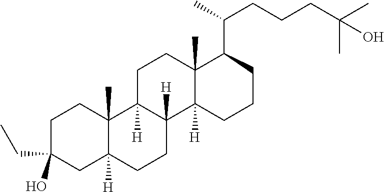 |
| H-5 | 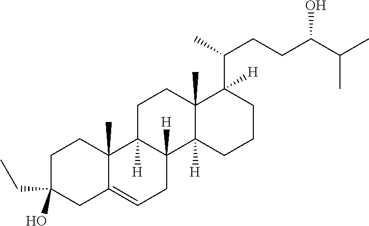 |
| H-6 | 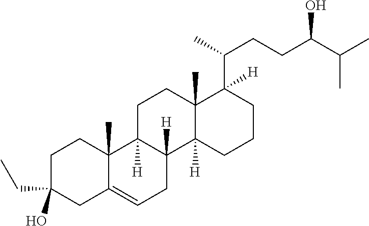 |
| H-7 | 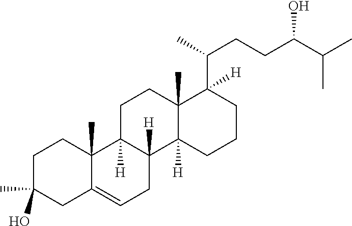 |
| H-8 | 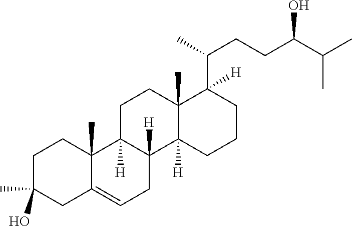 |
| H-9 | 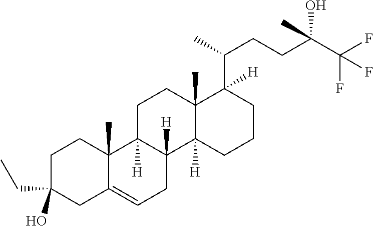 |
| H-10 | 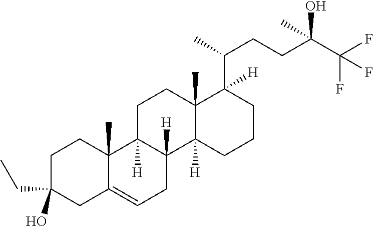 |
| H-11 | 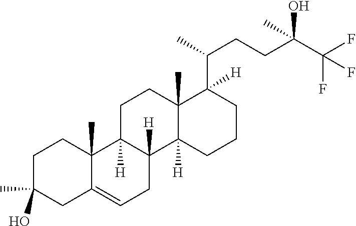 |

and pharmaceutically acceptable salts thereof.

17. A pharmaceutical composition comprising a compound or pharmaceutically acceptable salt thereof according to any one of claims 1-16, and a pharmaceutically acceptable carrier.

18. A method for treating a CNS-related condition in a subject comprising administering to the subject an effective amount of a compound or pharmaceutically acceptable salt thereof according to any one of claims 1-16, or a pharmaceutical composition according to claim 17.

19. The method according to claim 18, wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease), and tinnitus.

20. A method of inducing sedation or anesthesia in a subject, comprising administering to the subject an effective amount of a compound or pharmaceutically acceptable salt thereof according to any one of claims 1-16, or a pharmaceutical composition according to claim 17.

21. A compound or pharmaceutically acceptable salt thereof according to any one of claims 1-16, or a pharmaceutical composition according to claim 17, for use in treating a CNS-related condition in a subject.

22. The compound or pharmaceutically acceptable salt thereof, or pharmaceutical composition for use according to claim 21, wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease), and tinnitus.

23. A compound or pharmaceutically acceptable salt thereof according to any one of claims 1-16, or a pharmaceutical composition according to claim 17, for use in inducing sedation or anesthesia in a subject.

24. Use of a compound or pharmaceutically acceptable salt thereof according to any one of claims 1-16, or a pharmaceutical composition according to claim 17, for the manufacture of a medicament for treating a CNS-related condition in a subject.

25. The use according to claim 24, wherein the CNS-related condition is selected from the group consisting of an adjustment disorder, anxiety disorder (including obsessive-compulsive disorder, posttraumatic stress disorder, and social phobia), cognitive disorder (including Alzheimer's disease and other forms of dementia), dissociative disorder, eating disorder, mood disorder (including depression, bipolar disorder, and dysthymic disorder), schizophrenia or other psychotic disorder (including schizoaffective disorder), sleep disorder (including insomnia), substance-related disorder, personality disorder (including obsessive-compulsive personality disorder), autism spectrum disorders (including those involving mutations to the Shank group of proteins), neurodevelopmental disorder (including Rett syndrome, Tuberous Sclerosis complex), pain (including acute and chronic pain), encephalopathy secondary to a medical condition (including hepatic encephalopathy and anti-NMDA receptor encephalitis), seizure disorder (including status epilepticus and monogenic forms of epilepsy such as Dravet's disease), stroke, traumatic brain injury, movement disorder (including Huntington's disease and Parkinson's disease), and tinnitus.

26. Use of a compound or pharmaceutically acceptable salt thereof according to any one of claims 1-16, or a pharmaceutical composition according to claim 17, for the manufacture of a medicament for inducing sedation or anesthesia in a subject.
